(19) United States
(12) Patent Application Publication
(10) Pub. No.: US 2022/0370416 A1
(43) Pub. Date: Nov. 24, 2022

(54) COMPOUNDS AND METHODS FOR TARGETED DEGRADATION OF KRAS

(71) Applicants: Arvinas Operations, Inc., New Haven, CT (US); Yale University, New Haven, CT (US)

(72) Inventors: Ling Chu, Anhui (CN); Craig M. Crews, New Haven, CT (US); Hanqing Dong, Madison, CT (US); Keith R. Hornberger, Southbury, CT (US); Jesus Raul Medina, Gilbertsville, PA (US); Lawrence Snyder, Killingworth, CT (US); Jing Wang, Milford, CT (US)

(21) Appl. No.: 17/223,551

(22) Filed: Apr. 6, 2021

Related U.S. Application Data
(60) Provisional application No. 63/030,440, filed on May 27, 2020.
(60) Provisional application No. 63/006,000, filed on Apr. 6, 2020.

(51) Int. Cl. A61K 31/427; A61K 47/54; A61K 45/06; A61K 31/422; A61K 31/4155

(52) U.S. Cl. CPC A61K 31/427; A61K 47/545; A61K 31/4155; A61K 31/422; A61K 45/06

(57) ABSTRACT

Bifunctional compounds, which find utility as modulators of Kirsten ras sarcoma protein (KRas or KRAS), are described herein. In particular, the hetero-bifunctional compounds of the present disclosure contain on one end a moiety that binds to the Von Hippel-Lindau E3 ubiquitin ligase and on the other end a moiety which binds KRas, such that the target protein is placed in proximity to the ubiquitin ligase to effect degradation (and inhibition) of target protein. The hetero-bifunctional compounds of the present disclosure exhibit a broad range of pharmacological activities associated with degradation/inhibition of target protein. Diseases or disorders that result from aberrant regulation of the target protein are treated or prevented with compounds and compositions of the present disclosure.

### CROSS-REFERENCE TO RELATED APPLICATIONS

The present disclosure claims benefit of and priority to U.S. Provisional Application No. 63/006,000, filed 6 Apr. 2020, titled COMPOUNDS AND METHODS FOR TARGETED DEGRADATION OF KRAS, and U.S. Provisional Application No. 63/030,440, filed 27 May 2020, titled COMPOUNDS AND METHODS FOR TARGETED DEGRADATION OF KRAS, which is incorporated herein in by reference in its entirety for all purposes.

### STATEMENT REGARDING FEDERALLY FUNDED RESEARCH

This invention was made with government support under grant number NIH R35CA197589 of the National Institutes of Health. The government has certain rights in this invention.

### INCORPORATION BY REFERENCE

All cited references are hereby incorporated herein by reference in their entirety, including U.S. patent application Ser. No. 15/074,820, filed on 18 Mar. 2016, published as U.S. Patent Application Publication No. 2016/0272639; and U.S. patent application Ser. No. 14/371,956, filed on 11 Jul. 2014, published as U.S. Patent Application Publication No. 2014/0356322; and U.S. patent application Ser. No. 16/224,088, filed on 18 Dec. 2018, published as U.S. Patent Application Publication No. 2019/0127359; and U.S. patent application Ser. No. 16/375,643, filed on 4 Apr. 2019, published as U.S. Patent Application Publication No. 2019/0315732.

### FIELD OF THE INVENTION

The invention provides hetero-bifunctional compounds comprising a target protein binding moiety and an E3 ubiquitin ligase binding moiety, and associated methods of use. The bifunctional compounds are useful as modulators of targeted ubiquitination of Kirsten ras sarcoma protein with a G12C mutation, which is then degraded and/or inhibited.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands that bind to these ligases. For example, since the discovery of nutlins, the first small molecule E3 ligase inhibitors, additional compounds have been reported that target E3 ligases.

Von Hippel-Lindau (VHL) tumor suppressor is the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. The first small molecule ligands of Von Hippel Lindau (VHL) to the substrate recognition subunit of the E3 ligase were generated, and crystal structures were obtained confirming that the compound mimics the binding mode of the transcription factor HIF-1α, the major substrate of VHL.

Bifunctional compounds such as those described in U.S. Patent Application Publications 2015/0291562 and 2014/0356322 (incorporated herein by reference), function to recruit endogenous proteins to an E3 ubiquitin ligase for ubiquitination and subsequent degradation in the proteasome degradation pathway. In particular, the publications cited above describe bifunctional or proteolysis-targeting chimeric (PROTAC®) protein degrader compounds, which find utility as modulators of targeted ubiquitination of a variety of polypeptides and proteins, which are then degraded and/or inhibited by the bifunctional compounds.

The Kirsten rat sarcoma (KRAS) gene is an oncogene encoding KRas, which is a small GTPase signal transduction protein. Ras proteins associate with the plasma membrane, and act as switches in the transduction of extracellular signals to intracellular response, thereby regulating, e.g., cell division. In normal cells, KRAS functions as a molecular switch, cycling between an inactive, GDP-bound “off” state and an active, GTP-bound “on” state (Milburn et al.; Ito, Y., et al., Regional polysterism in the GTP-bound form of the human c-Ha-Ras protein. Biochemistry 1997, 36 (30), 9109-9119). This switch is tightly regulated by guanine nucleotide exchange factor (GEF) proteins, which exchange GDP for GTP, and GTPase-activating proteins (GAPs), which enhance the intrinsically slow GTPase activity of KRAS (Bar-Sagi, D., The Sos (Son of sevenless) protein. Trends Endocrinol Metab 1994, 5 (4), 165-9; Pierre, S., et al., Understanding SOS (Son of Sevenless). Biochem Pharmacol 2011, 82 (9), 1049-56; Harrell Stewart, D. R., et al., Pumping the brakes on RAS—negative regulators and death effectors of RAS. J Cell Sci 2020, 133 (3)). GEF and GAP effector proteins bind at one or both of two shallow binding pockets on KRAS termed switch I (residues 30-38) and switch II (residues 59-76), the conformations of which change dramatically between GDP-bound state and GTP-bound state (Ito et al.; Boriack-Sjodin, P. A. et al., The structural basis of the activation of Ras by Sos. Nature 1998, 394 (6691), 337-43; Scheffzek, K. et al., The Ras-RasGAP complex: structural basis for GTPase activation and its loss in oncogenic Ras mutants. Science 1997, 277 (5324), 333-8).

The KRAS gene is one of the most frequently mutated oncogenes in cancer (Prior, I. A.; Lewis, P. D.; Mattos, C., A comprehensive survey of Ras mutations in cancer. Cancer Res 2012, 72 (10), 2457-67; Land, H.; Parada, L. F.; Weinberg, R. A., Tumorigenic conversion of primary embryo fibroblasts requires at least two cooperating oncogenes. Nature 1983, 304 (5927), 596-602; Newbold, R. F.; Overell, R. W., Fibroblast Immortality Is a Prerequisite for Transformation by Ej C-Ha-Ras Oncogene. Nature 1983, 304 (5927), 648-651). KRAS encodes a small, membrane bound GTPase that relays signals from receptor tyrosine kinases (RTKs), promoting cell proliferation, cell differentiation or cell death (Milburn, M. V., et al., Molecular Switch for Signal Transduction—Structural Differences between Active and Inactive Forms of Protooncogenic Ras Proteins. Science 1990, 247 (4945), 939-945; Simanshu, D. K., et al., RAS Proteins and Their Regulators in Human Disease. Cell 2017, 170 (1), 17-33). Somatic KRAS mutations attenuate the GAP-mediated enzymatic activity of the protein, resulting in accumulation of GTP-bound, active KRAS and hyperactivation of downstream signaling, which leads to uncontrolled cell proliferation (Prior et al.; Simanshu et al.). Numerous activating or gain-of-function mutations of the KRas gene are known, and in fact, KRas is the most frequently mutated gene in cancer. Gain-in-function KRas mutations are found in approximately 30% of all human cancers, including, e.g., pancreatic cancer (>80%), colon cancer (approximately 40-50%), lung cancer (approximately 30-50%), non-small cell lung cancer, biliary tract malignancies, endometrial cancer, cervical cancer, bladder cancer, liver cancer, myeloid leukemia, and breast cancer. These activating mutations impair the ability of KRas to switch between active and inactive states. Key roles for mutant KRas have been established in initiation, maintenance, progression, and metastasis of various cancers, and mutations are frequently correlated with poor prognosis and increased resistance to chemotherapy and biological therapies, including, e.g., therapies that target epidermal growth factor receptor. However, despite its key role and rates prevalence in cancer, there is an absence of effective therapies that directly target this oncogene, leading to it being considered “undruggable.” Furthermore, despite its prevalence in cancer and many years of extensive research efforts, mutant KRAS has remained a challenging therapeutic target given the scarcity of traditional druggable pockets on its surface (Spencer-Smith, R. et al., Direct inhibition of RAS: Quest for the Holy Grail? Semin Cancer Biol 2019, 54, 138-148).

The KRAS GJ2C mutation is highly prevalent in lung adenocarcinoma (LUAD). KRAS^{G12C} mutants make up over 50% of all KRAS mutant LUAD tumors (13% of total LUAD tumors) (Prior et al. 2012). Additionally, 3% of colorectal cancers and 1% of all other solid tumors express KRAS^{G12C} (Campbell, J. D., et al., Distinct patterns of somatic genome alterations in lung adenocarcinomas and squamous cell carcinomas. Nat Genet 2016, 48 (6), 607-16). This mutation greatly reduces KRAS's intrinsic GTPase activity, allowing for the accumulation of GTP-bound, active KRAS (Lu, S., et al., GTP Binding and Oncogenic Mutations May Attenuate Hypervariable Region (HVR)-Catalytic Domain Interactions in Small GTPase K-Ras4B, Exposing the Effector Binding Site. J Biol Chem 2015, 290 (48), 28887-900). Recent advances, initially led by the Shokat group, have identified molecules that covalently and selectively bind the mutated cysteine of KRAS^{G12C} (Ostrem, J. M., et al., K-Ras(G12C) inhibitors allosterically control GTP affinity and effector interactions. Nature 2013, 503 (7477), 548-51; Rudolph, J., et al., Selective inhibition of mutant Ras protein through covalent binding. Angew Chem Int Ed Engl 2014, 53 (15), 3777-9; Ostrem, J. M., et al., Direct small-molecule inhibitors of KRAS: from structural insights to mechanism-based design. Nat Rev Drug Discov 2016, 15 (11), 771-785; Nnadi, C. I., et al., Novel K-Ras G12C Switch-II Covalent Binders Destabilize Ras and Accelerate Nucleotide Exchange. J Chem Inf Model 2018, 58 (2), 464-471). These compounds induce a novel, drug-like pocket within the KRAS switch II region (Ostrem et al. 2013). Optimization of the electrophiles responsible for conjugating the cysteine as well as the molecular interactions within the drug-induced pocket have led to the development of orally bioavailable KRAS^{G12C} inhibitors. However, despite this success, rapid adaptive resistance and MAPK signaling reactivation after inhibitor treatment have already been reported (Ryan, M. B. et al., Vertical Pathway Inhibition Overcomes Adaptive Feedback Resistance to KRAS(G12C) Inhibition. Clin Cancer Res 2019; Xue, J. Y., et al., Rapid non-uniform adaptation to conformation-specific KRAS(G12C) inhibition. Nature 2020, 577 (7790), 421-425). Thus, the development of complementary therapeutic strategies could help realize the full potential of targeting KRAS mutants for cancer treatment.

An ongoing need exists in the art for effective treatments for KRas related disease and disorders, e.g., pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

### SUMMARY

The present disclosure describes hetero-bifunctional compounds that function to recruit Kirsten ras sarcoma protein (KRas or KRAS), such as mutant or gain-of-function KRas, to an E3 ubiquitin ligase for targeted ubiquitination and subsequent proteasomal degradation, and methods of making and using the same. In addition, the description provides methods of using an effective amount of a compound of the present disclosure for the treatment or amelioration of a disease condition, such as a KRas-related disease or disorder, e.g., accumulation or overactivity of an KRas protein or a mutated or gain-of function KRas protein or a mis-folded KRas protein, pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

As such, in one aspect the disclosure provides hetero-bifunctional compounds, which comprise an E3 ubiquitin ligase binding moiety (i.e., a ligand for an E3 ubiquitin ligase (a “ULM” group)), and a moiety that binds KRas or a mutated version thereof (i.e., a protein targeting moiety or “PTM” group, that is, a KRas targeting ligand or a “KTM” group) such that the KRas protein is thereby placed in proximity to the ubiquitin ligase to effect ubiquitination and subsequent degradation (and/or inhibition) of the KRas protein. In a preferred embodiment, the ULM (ubiquitination ligase binding moiety) is a Von Hippel-Lindau (VHL) E3 ubiquitin ligase binding moiety (VLM). For example, the structure of the bifunctional compound can be depicted as:

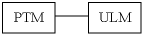

The respective positions of the PTM and ULM moieties (e.g., VLM), as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

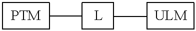

where PTM is a KRas-targeting moiety (KTM), L is a linker, e.g., a bond or a chemical linking group coupling PTM to ULM, and ULM is a VHL E3 ubiquitin ligase binding moiety (VLM).

For example, the structure of the bifunctional compound can be depicted as:

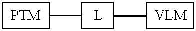

wherein: PTM is a KRas-targeting moiety (KTM); “L” is a linker (e.g. a bond or a chemical linking group) coupling the PTM and VLM; and VLM is a VHL E3 ubiquitin ligase binding moiety that binds to VHL E3 ubiquitin ligase.

In certain embodiments, the compounds as described herein comprise multiple independently selected ULMs, multiple PTMs, multiple chemical linkers or a combination thereof.

In any of the aspects or embodiments described herein, the PTM is a small molecule that binds KRas or a mutant thereof, such as a gain-of-function KRas. In any of the aspects or embodiments described herein, the PTM is a small molecule that binds KRas. In any of the aspects or embodiments described herein, the PTM is a small molecule that binds both a KRas wild type protein and a KRas mutant, such as a KRas protein that has gain-of-function mutation. In any of the aspects or embodiments described herein, the PTM is a small molecule that binds both an KRas wild type protein and an KRas mutant such as, but not limited to, a gain-of-function KRas mutant. In any aspect or embodiment described herein, the small molecule binds the KRas is as described herein.

In an embodiment, the VLM is a derivative of trans-3-hydroxyproline, where both nitrogen and carboxylic acid in trans-3-hydroxyproline are functionalized as amides. Other contemplated VLMs are described in U.S. Patent Application Publication No. 2016/0272639, U.S. Patent Application Publication No. 2014/0356322, each of which is incorporated herein by reference in its entirety.

In certain embodiments, “L” is a bond. In additional embodiments, the linker “L” is a connector with a linear non-hydrogen atom number in the range of 1 to 40 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40). The connector “L” can contain, but is not limited to one or more functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic or tricyclic moieties. Substitution with halogen, such as Cl, F, Br and I, or alkyl, such as methyl, ethyl, isopropyl, and tert-butyl, can be included in the linker. In the case of fluorine substitution, single or multiple fluorines can be included.

In an additional aspect, the description provides therapeutic compositions comprising an effective amount of a compound as described herein, or a salt form thereof, and a pharmaceutically acceptable carrier. The therapeutic compositions can be used to trigger targeted degradation of KRas or a mutated version thereof and/or inhibition of KRas or a mutated version thereof, in a patient or subject, for example, an animal such as a human, and can be used for treating or ameliorating one or more disease states, conditions, or symptoms causally related to KRas or mutated version thereof, which treatment is accomplished through degradation or inhibition of the KRas protein or mutated version thereof, or controlling or lowering KRas protein levels or protein levels of a mutated version thereof, in a patient or subject. In certain embodiments, the therapeutic compositions as described herein may be used to effectuate the degradation of KRas, or a mutant or mis-folded form thereof, for the treatment or amelioration of a disease such as, e.g., accumulation, aggregation, or overactivity of a KRas protein, a mis-folded, or a mutated form thereof (such as a gain-of-function KRas protein, pancreatic cancer, colon cancer, colorectal cancer, lung cancer, non-small cell lung cancer, biliary tract malignancies, endometrial cancer, cervical cancer, bladder cancer, liver cancer, myeloid leukemia, or breast cancer.

In yet another aspect, the present disclosure provides a method of ubiquitinating KRas or a mutated form thereof in a cell (e.g., in vitro or in vivo). In any aspect or embodiment described herein, the method comprises administering a hetero-bifunctional compound as described herein comprising a PTM that binds KRas or a mutant form thereof, and a VLM, preferably linked through a chemical linker moiety, as described herein, to effectuate degradation of the KRas protein or mutant form thereof. Though not wanting to be limited by theory, the inventors believe that, pursuant to the invention, poly-ubiquitination of the KRas wild-type or mutant protein will occur when it is placed in proximity to the E3 ubiquitin ligase via use of the hetero-bifunctional compound, thereby triggering subsequent degradation of the KRas or mutant protein via the proteasomal pathway and control or reduction of KRas protein levels in cells, such as cells of a subject in need of such treatment. The control or reduction in levels of the KRas protein or mutated form thereof afforded by the present disclosure provides treatment of a KRas causally related disease state, condition or related symptom, as modulated through a lowering of the amount of KRas protein or mutated form thereof in cells of the subject.

In still another aspect, the description provides methods for treating or ameliorating a disease, condition, or symptom thereof causally related to KRas or mutated form thereof in a subject or a patient, e.g., an animal such as a human, comprising administering to a subject in need thereof a composition comprising an effective amount, e.g., a therapeutically effective amount, of a hetero-bifunctional compound as described herein or salt form thereof, and a pharmaceutically acceptable carrier, wherein the composition is effective for treating or ameliorating the disease or disorder or symptom thereof in the subject.

In any aspect or embodiment described herein, the method further includes, prior to administering a composition or compound of the present disclosure to a subject, identifying a patient as having a mutant KRas protein (e.g., KRas^{G12C}).

In another aspect, the description provides methods for identifying the effects of the degradation of KRas protein in a biological system using compounds according to the present disclosure.

In another aspect, the description provides processes and intermediates for making a hetero-bifunctional compound of the present disclosure capable of targeted ubiquitination and degradation of the KRas protein in a cell (e.g., in vivo or in vitro).

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure. Further objects, features and advantages of the disclosure will become apparent from the following detailed description taken in conjunction with the accompanying figures showing illustrative embodiments of the disclosure.

FIGS. 1A and 1B. Illustration of general principle for hetero-bifunctional protein-degrading compounds. FIG. 1A. Exemplary hetero-biofunctional protein degrading compounds comprise a protein targeting moiety (PTM; darkly shaded rectangle), a ubiquitin ligase binding moiety (ULM; lightly shaded triangle), and optionally a linker moiety (L; black line) coupling the PTM to the ULM. FIG. 1B Illustrates the functional use of the hetero-bifunctional protein degrading compounds (commercially known as PROTAC® protein degrader compounds) as described herein. Briefly, the ULM (triangle) recognizes and binds to a specific E3 ubiquitin ligase, and the PTM (large rectangle) binds and recruits a target protein bringing it into close proximity to the E3 ubiquitin ligase. Typically, the E3 ubiquitin ligase is complexed with an E2 ubiquitin-conjugating protein (E2), and either alone or via the E2 protein catalyzes attachment of multiple ubiquitin molecules (black circles) to a lysine on the target protein via an isopeptide bond. The poly-ubiquitinated protein (far right) has thereby been targeted for degradation by the proteosomal machinery of the cell.

FIGS. 2A, 2B, and 2C. MRTX849-VHL bifunctional compounds engage and degrade endogenous KRAS^{G12C} in NCI-H2030 cells: (2A) Chemical structures of MRTX849, LC-1, LC-2 (active bifunctional protein-degrading compound), and LC-2 Epimer. (2B) LC-1 engages KRAS^{G12C} in a dose dependent manner. Quantitation on the right. (2C) LC-2 degrades KRAS^{G12C} in a dose dependent manner. Quantitation on the right. Quantified data represents mean±SD. Not Significant (N.S.); * p<0.05; ** p<0.01; **** p<0.001.

FIGS. 3A and 3B. Docking of MRTX849 and LC-2 degradation is specific for KRAS^{G12C}. (3A) Docking of MRTX849 (MRTX) into the crystal structure of KRAS^{G12C} (PDB: 5V9U). MRTX is shown in yellow, the black arrow indicates the point of linker attachment. (3B) LC-2 does not degrade KRAS^{G12D} in HCT116 cells. Quantitation on the right. Quantitation on the right. Quantified data represents mean±SD.

FIGS. 4A, 4B, and 4C. LC-2 induces KRAS^{G12C} degradation in multiple mutant cell lines. (4A) LC-2, but not LC-2 Epimer, induces KRAS^{G12C} degradation in heterozygous H358 cells. Quantitation on the right. (4B) LC-2 induces KRAS^{G12C} in homozygous MIA PaCa-2 cells. Quantitation on the right. (4C) LC-2 induces KRAS^{G12C} degradation in the homozygous, MRTX849 resistant, SW1573 cells. (4D) LC-2 induces KRAS^{G12C} degradation in heterozygous NCI-H23. Quantitation on the right. Quantified data represents mean±SD. * p<0.05; ** p<0.01; *** p<0.005; **** p<0.001.

FIGS. 5A and 5B. Degradation of endogenous KRAS^{G12C} is via the heterobifunctional compound. (5A) LC-2 Epimer does not induce KRAS^{G12C} degradation at 2.5 μM and LC-2 induced degradation is rescued by VHL ligand competition, proteasome inhibition with epoxomicin (Epox), and neddylation inhibition with MLN924 (MLN), in NCI-H2030 cells. Quantitation is below. (5B) Inhibition of neddylation, but not inhibition of lysosomal acidification, rescues LC-2 induced KRAS^{G12C} degradation in NCI-H23 cells. Quantitation is below. Quantified data represents mean±SD. Not Significant (N.S.); *** p<0.005.

FIGS. 6A and 6B. KRAS^{G12C} degradation is rapid, with maximal degradation induced as early as 4 hours: (6A) Time course in NCI-H2030 cells. LC-2 and LC-2 epimer engage within 1 hour with maximal degradation observed by 8 hours and maintained up to 24 hours. Quantitation on the right. (6B) Time course in SW1573 cells. LC-2 and LC-2 epimer engage KRAS within 1 hour and maximal degradation is observed at 12 hours and maintained up to 24 hours. Quantitation on the right. LC-2 Epimer is a quantification of the higher molecular weight, bifunctional compound Epimer modified band to monitor engagement of KRAS^{G12C} overtime rather than total KRAS levels. Quantified data represents mean±SD. Not Significant (N.S.); * p<0.05; ** p<0.01; *** p<0.005; **** p<0.001.

FIGS. 7A and 7B: Degradation of endogenous KRAS^{G12C} is sustained over 72 hours in multiple cancer cell lines. (7A) 72 hour time course in MIA PaCa-2 cells. Degradation occurs at 6 hours and is maintained up to 72 hours. Quantitation on the right. (7B) 72 hour time course in NCI-H23 cells. Degradation occurs within 6 hours, reaches a maximum at 24 hours, and begins to rebound by 72 hours. Quantitation on the right. Quantified data represents mean±SD. Not Significant (N.S.); ** p<0.01; *** p<0.005; **** p<0.001

FIG. 8: LC-2 induced KRAS^{G12C} degradation is maintained over 72 hours in SW1573. LC-2 induced KRAS^{G12C} occurs within 6 hrs and is maintained for 72 hours. No change is observed for LC-2 Epimer.

FIGS. 9A and 9B: Degradation of endogenous KRAS^{G12C} modulates Erk signaling in homozygous and heterozygous KRAS^{G12C} cell lines. (9A) Degradation of KRAS^{G12C} in homozygous NCI-H2030 cells attenuates pErk in a dose dependent manner. Quantitation on the right. (9B) Degradation of KRAS^{G12C} in heterozygous NCI-H23 cells decreases pErk in a dose dependent manner. Quantitation on the right. For statistical analysis see Tables 2 and 3. Quantified data represents mean±SD.

FIGS. 10A and 10B: Effect of KRAS^{G12C} degradation and inhibition on Erk signaling over time. (10A) Inhibition and degradation of KRAS^{G12C} decreases pErk signaling at 6 and 24 hrs in homozygous MIA PaCa-2 cells. Quantitation on the right. (10B) Inhibition and degradation of KRAS^{G12C} decreases pErk signaling at 6 and 24 hrs in heterozygous NCI-H23. Quantitation on the right. For statistical analysis see Tables 4 and 5. Quantified data represents mean±SD.

FIG. 11: Changes in Erk signaling during a 24 hour LC-2 treatment in SW1573 cells. Erk signaling is modulated by LC-2. pErk is decreased throughout the time course. Quantitation on the right. Quantified data represents mean±SD. For statistical analysis see Table 6.

FIG. 12: Chemical structures, linker length (from the carbon adjacent to the pyrrolidine nitrogen to the carbon adjacent to the VHL carbonyl), and activity are presented for exemplary compounds LC-1, LC-2, LC-3, LC-4, LC-5, and LC-6. Shorter linker lengths induce higher levels of degradation. ^{a}Data from NCI-H2030 cells. ^{b}Data from SW1573 cells.

### DETAILED DESCRIPTION

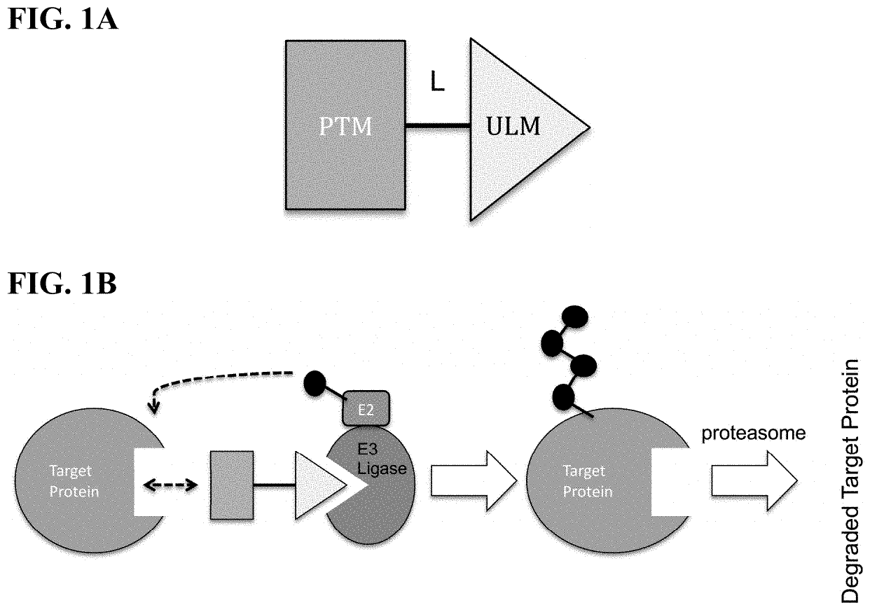

Presently described are compounds, compositions and methods that relate to the surprising discovery that an E3 ubiquitin ligase (e.g., a Von Hippel-Lindau (VHL) E3 ubiquitin ligase) ubiquitinates the KRas protein or mutated form thereof once the E3 ubiquitin ligase and the KRas protein are placed in proximity via a bifunctional compound that binds both the E3 ubiquitin ligase and the KRas protein. Accordingly the present disclosure provides compounds and compositions comprising an E3 ubiquitin ligase binding moiety (“ULM”) coupled by a bond or chemical linking group (L) to a protein targeting moiety (“PTM”) that targets the KRas protein, which results in the ubiquitination of the KRas protein, and which leads to degradation of the KRas protein by the proteasome (see FIGS. 1A and 1B).

In an aspect, the description provides compounds in which the PTM binds to the KRas protein and/or a mutated form thereof. The present disclosure also provides a library of compositions and the use thereof to produce targeted degradation of the KRas protein in a cell.

In certain aspects, the present disclosure provides hetero-bifunctional compounds which comprise a ligand, e.g., a small molecule ligand (i.e., having a molecular weight of below 2,000, 1,000, 500, or 200 Daltons), which is capable of binding to an E3 ubiquitin ligase, such as the Von Hippel-Lindau E3 ubiquitin ligase. The compounds also comprise a small molecule moiety that is capable of binding to the KRas protein or mutated form thereof in such a way that the KRas protein or mutated form is placed in proximity to the ubiquitin ligase to effect ubiquitination and degradation (and/or inhibition) of the KRas protein or mutated form. “Small molecule” means, in addition to the above, that the molecule is non-peptidyl, that is, it is not considered a peptide, e.g., comprises fewer than 4, 3, or 2 amino acid residues. In accordance with the present description, each of the PTM, ULM and hetero-bifunctional molecule is a small molecule.

The term “KRas” as used throughout the Specification, unless specifically indicated to the contrary, is intended to include both wild-type KRas and mutant forms therefore, such as a gain-of-function KRas mutant protein or a KRas protein having one or more mutation selected from codon 12 missense mutation, codon 12 missense mutation, exon 2 mutation, G12V, G12C, G12D, G12A, G13D, exon 3 mutation, codon 61 missense mutation, exon 4 mutation, G12R, Q61H, G12S, A146T, G13C, Q61R, Q61L, A146V, codon 117 missense mutation, K117N, Q61K, G12F, codon 59 mis sense mutation, A59T, or combinations thereof or combinations thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value in the range, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either/or both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element, unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, in certain methods or processes described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time-varied administration (administration of one or more therapeutic agents at a time different from that of the administration of an additional therapeutic agent or agents), as long as the two or more therapeutic agents are present in the patient to some extent, preferably at effective amounts, at the same time. In certain preferred aspects, one or more of the hetero-bifunctional compounds described herein are coadministered with at least one additional bioactive agent, e.g., an anticancer agent. In particularly preferred aspects, the co-administration of such compounds results in synergistic activity and/or therapy such as, e.g., anticancer activity.

The term “compound”, as used herein, unless otherwise indicated, refers to any specific hetero-bifunctional compound disclosed herein, pharmaceutically acceptable salts and solvates thereof, and deuterated forms of any of the aforementioned molecules, where applicable. Deuterated compounds contemplated are those in which one or more of the hydrogen atoms contained in the drug molecule have been replaced by deuterium. Such deuterated compounds preferably have one or more improved pharmacokinetic or pharmacodynamic properties (e.g., longer half-life) compared to the equivalent “undeuterated” compound.

The term “ubiquitin ligase” refers to a family of proteins that facilitate the transfer of one or more ubiquitins to a specific substrate protein. Addition of a chain of several ubiquitins (poly-ubiquitination) targets the substrate protein for degradation. For example, Von Hippel-Lindau is an E3 ubiquitin ligase that alone, or in combination with an E2 ubiquitin-conjugating enzyme, can ultimately cause the attachment of a chain of four ubiquitins to a lysine residue on the target protein, thereby targeting the protein for degradation by the proteasome. The ubiquitin ligase is involved in poly-ubiquitination such that a first ubiquitin is attached to a lysine on the target protein; a second ubiquitin is attached to the first; a third is attached to the second, and a fourth is attached to the third. Such poly-ubiquitination marks proteins for degradation by the proteasome.

The term “patient” or “subject” is used throughout the specification to describe an animal, preferably a human or a domesticated animal, to whom treatment, including prophylactic treatment, with the compositions according to the present disclosure is provided. For treatment of those diseases, conditions or symptoms that are specific for a specific animal, such as a human patient, the term “patient” refers to that specific animal, including a domesticated animal such as a dog or cat, or a farm animal such as a horse, cow, sheep, etc. In general, in the present disclosure, the terms “patient” and “subject” refer to a human patient unless otherwise stated or implied from the context of the use of the term.

The terms “effective” and “therapeutically effective” are used to describe an amount of a compound or composition which, when used within the context of its intended use, and either in a single dose or, more preferably after multiple doses within the context of a treatment regimen, effects an intended result such as an improvement in a disease or condition, or amelioration or reduction in one or more symptoms associated with a disease or condition. The terms “effective” and “therapeutically effective” subsume all other “effective amount” or “effective concentration” terms, which are otherwise described or used in the present application.

Compounds and Compositions

In one aspect, the description provides hetero-bifunctional compounds comprising an E3 ubiquitin ligase binding moiety (“ULM”) that is a VHL E3 ubiquitin ligase binding moiety (a “VLM”), The VLM is covalently coupled to a protein targeting moiety (PTM) that binds to the protein, which coupling is either directly by a bond or via a chemical linking group (L) according to the structure:

(A) PTM-L-VLM

wherein L is the bond or chemical linking group, and PTM is a protein targeting moiety that binds to the protein KRas or a mutant form thereof, as described herein, where the PTM is a KRas targeting moiety (KTM). The term VLM is inclusive of all VHL binding moieties.

In any of the aspects or embodiments, the VLM demonstrates a half maximal inhibitory concentration (IC₅₀) for the E3 ubiquitin ligase (e.g., VHL E3 ubiquitin ligase) of less than about 200 μM. The IC₅₀ can be determined according to any suitable method known in the art, e.g., a fluorescent polarization assay.

In certain embodiments, the hetero-bifunctional compounds described herein demonstrate an IC₅₀ or a half maximal degradation concentration (DC₅₀) of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 pM.

In any aspect or embodiment described herein, the PTM is represented by the chemical structure:

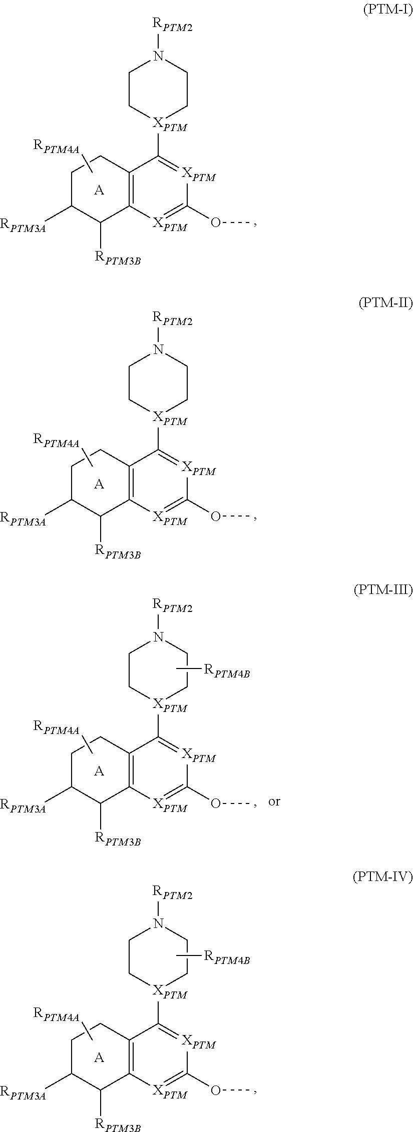

wherein:
the [symbol] of the PTM is the site of attachment to the VLM or the L coupling the VLM to the PTM;

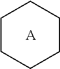

is an 6-membered aryl, 6-membered heteroaryl, or a 6-membered heterocycloalkyl, each optionally substituted with 1 or 2 halogens (e.g., Cl, F, or Br);
R_{PTM2} is —C(═O)C2-C4alkenyl, optionally substituted by a methyl or halogen (e.g., Cl, F, Br); R_{PTM3A} is H, phenyl, or naphthalene, each optionally substituted by 1, 2, or 3 groups independently selected from OH, halogen (e.g., F, Cl, Br), or a linear or branched C1-C3 alkyl (e.g., methyl or ethyl);
R_{PTM3B} is H, halogen (e.g., Cl, F, Br), or —O—R_{PTM3C}, wherein R_{PTM3C} is an indazole

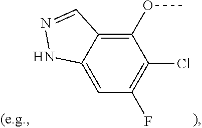

wherein R_{PTM3B} is optionally substituted by 1, 2, or 3 groups independently selected from OH, halogen (e.g., F, Cl, Br), or a linear or branched C1-C3 alkyl (e.g., methyl or ethyl);
R_{PTM4A} is 1 or 2 independently selected halogen (e.g., Cl, F, Br);
R_{PTM4B} is (1) —CH₂—CH₂—CN or —CH₂—CN, or (2) 1 or 2 independently selected C1-C3 alkyl (e.g., methyl or ethyl); and
each X_{PTM} is individually a CH or N.

The term “alkyl” shall mean within its context a linear, branch-chained or cyclic fully saturated hydrocarbon radical, preferably a C₁-C₁₀, preferably a C₁-C₆, or more preferably a C₁-C₃ alkyl group, which may be optionally substituted with any suitable functional group or groups. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylethyl, cyclohexylethyl and cyclohexyl, among others. In certain embodiments, the alkyl group is end-capped with a halogen group (At, Br, Cl, F, or I).

The term “Alkenyl” refers to linear, branch-chained or cyclic C₂-C₁₀ (preferably C₂-C₆) hydrocarbon radicals containing at least one C═C bond.

The term “Alkynyl” refers to linear, branch-chained or cyclic C₂-C₁₀ (preferably C₂-C₆) hydrocarbon radicals containing at least one C≡C bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other suitable functional group) which may be further substituted with a polyethylene glycol chain (of from 1 to 10, preferably 1 to 6, or more preferably 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (e.g., methylene) group, may be substituted with an amino acid sidechain group such as a sidechain group of a natural or unnatural amino acid, for example, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C₆ includes carbons atoms of 1, 2, 3, 4, 5 and 6 and for C₀, H stands in place of carbon.

The term “substituted” or “optionally substituted” shall mean independently (i.e., where more than one substituent occurs, each substituent is selected independent of another substituent) one or more substituents (independently up to five substituents, preferably up to three substituents, more preferably 1 or 2 substituents on a moiety in a compound according to the present disclosure and may include substituents which themselves may be further substituted) at a carbon (or nitrogen) position anywhere on a molecule within context, and includes as possible substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl, for example benzyl or benzoyl), alkoxy group (preferably, C₁-C₆ alkyl or aryl, including phenyl and substituted phenyl), thioether (preferably, C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which are preferably independently substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of Ri and R₂ is as otherwise described herein and R₃ is H or a C₁-C₆ alkyl group, preferably Ri, R₂, R₃ together is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteroaryl moiety) through an optionally substituted —(CH₂)ₘ— alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a side chain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ— (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂—R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical (e.g., a 5-16 membered ring) having a single ring (e.g., benzene, phenyl, benzyl, or 5, 6, 7 or 8 membered ring) or condensed rings (e.g., naphthyl, anthracenyl, phenanthrenyl, 10-16 membered ring, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (monocyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀- C6)alkyl, amine, mono- or di-(C₁-C₆ alkyl) amine wherein the alkyl group on the amine is optionally substituted with 1 or 2 hydroxyl groups or up to three halo (preferably F, C1) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably connected to a PTM group, including a ULM group, via a linker group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methyl substituted isoxazole, an optionally substituted oxazole including a methyl substituted oxazole, an optionally substituted thiazole including a methyl substituted thiazole, an optionally substituted isothiazole including a methyl substituted isothiazole, an optionally substituted pyrrole including a methyl substituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo- (preferably, F) or methyl substituted pyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to a 5-16 membered heteroaryl (e.g., 5, 6, 7 or 8 membered monocyclic ring or a 10-16 membered heteroaryl having multiple condensed rings), an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

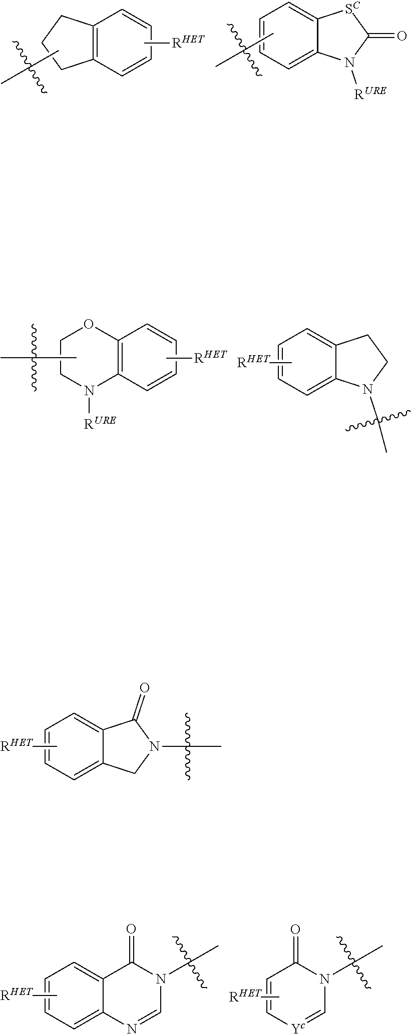

wherein:
S^{c} is CHR^{SS}, NR^{URE}, or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{c} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO-heteroaryl, —SO2-alkyl, —SO2-substituted alkyl, —SO2-aryl, oxo (═O), and —SO2-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “lower alkyl” refers to methyl, ethyl or propyl

The term “lower alkoxy” refers to methoxy, ethoxy or propoxy.

Exemplary VLMs

In any aspect or embodiment described herein, the ULM is a VLM and is represented by the chemical structure:

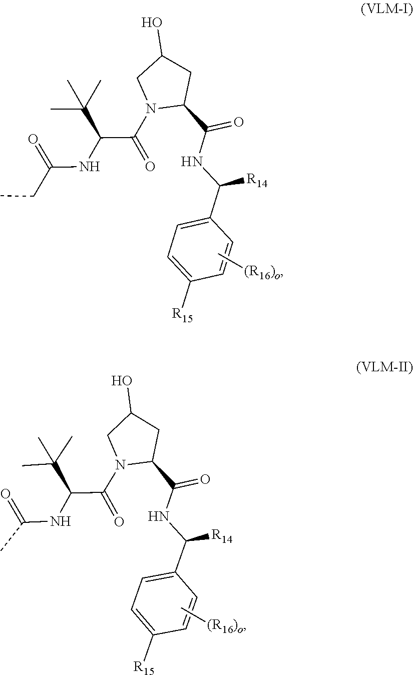

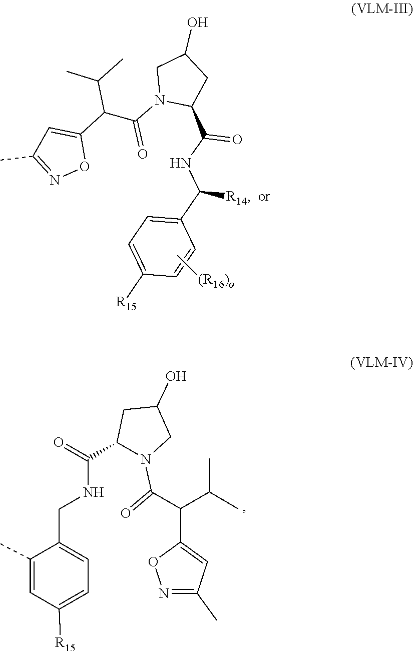

wherein:
R₁₄ is as defined in R₁₄, R₁₄ₐ, or R_{14b} in any aspect or embodiment described herein;
R₁₅ is as defined in any aspect or embodiment described herein;
R₁₆ is as defined in any aspect or embodiment described herein;
is as defined in any aspect or embodiment described herein; and
the [symbol] indicates the site of attachment of at least one PTM, another ULM (ULM′) or a chemical linker moiety coupling at least one PTM or a ULM′ or both to ULM.

For example, in any aspect or embodiment described herein, the ULM is a VLM and is represented by the chemical structure:

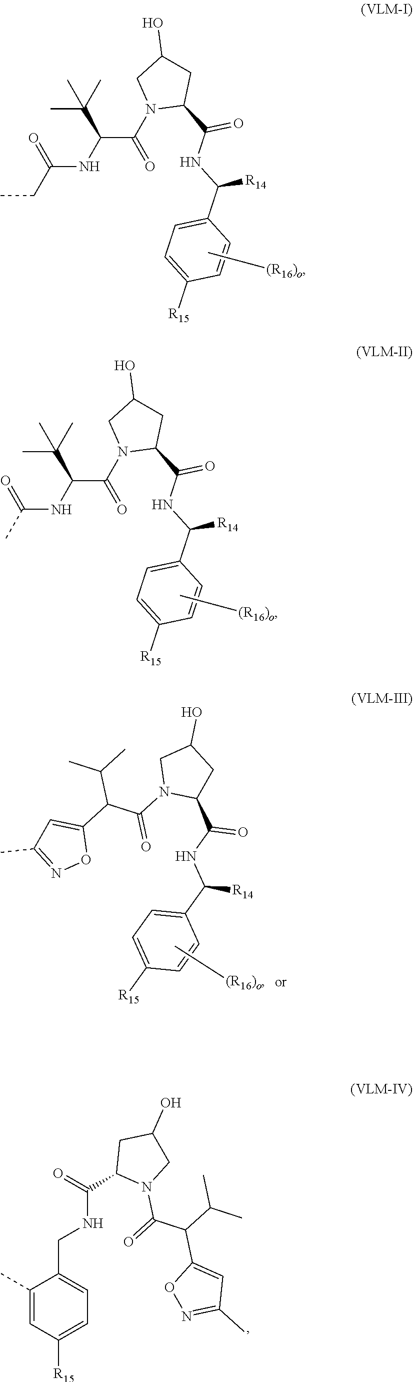

wherein:
R₁₄ is H or a linear or branched C₁-C₃ alkyl (e.g., methyl);
R₁₅ is a CN or a 5-membered heteroaryl having one or two heteroatoms selected from N, S, and O, optionally substituted with a methyl

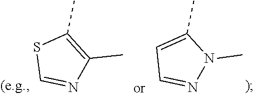

R₁₆ is a halo, optionally substituted C₁-C₃ alkyl, optionally substituted C₁-C₃ haloalkyl, hydroxy, optionally substituted C₁-C₃ alkoxy, or optionally substituted C₁-C₃ haloalkoxy;
is an integer from 0-2 (e.g., 0, 1, or 2); and
   the [symbol] indicates the site of attachment of at least one PTM, another ULM (ULM′) or a chemical linker moiety coupling at least one PTM or a ULM′ or both to ULM.

In any aspect or embodiment described herein, ULM is VLM and comprises a chemical structure selected from the group ULM-a:

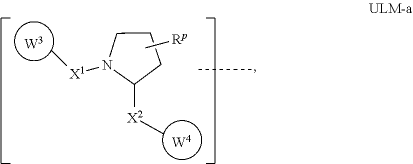

wherein:
the [symbol] indicates the attachment of at least one PTM, another ULM or VLM (i.e., ULM′ or VLM′), or a chemical linker moiety coupling at least one PTM, a ULM′ or a VLM′ to the other end of the linker;
X¹, X² of Formula ULM-a are each independently selected from the group of a bond, O, NR^{Y3}, CR^{Y3}R^{Y4}, C═O, C═S, SO, and SO₂;
R^{Y3}, R^{Y4} of Formula ULM-a are each independently selected from the group of H, linear or branched C₁₋₆ alkyl, optionally substituted by 1 or more halo, optionally substituted C₁₋₆ alkoxyl (e.g., optionally substituted by 0-3 R^{P} groups);
R^{P} of Formula ULM-a is 0, 1, 2, or 3 groups, each independently selected from the group H, halo, —OH, C1-3 alkyl, C═O;
W³ of Formula ULM-a is selected from the group of an optionally substituted T, an optionally substituted -T-N(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl, or an optionally substituted —NR¹-T-Heterocycle;
X³ of Formula ULM-a is C═O, R₁, R^{1a}, R^{1b};
each of R¹, R^{1a}, R^{1b} is independently selected from the group consisting of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T of Formula ULM-a is selected from the group of an optionally substituted alkyl, —(CH₂)ₙ— group, —(CH₂)ₙ—O—C₁-C₆ alkyl which is optionally substituted, linear, branched, or —(CH₂)ₙ—O-heterocyclyl which is optionally substituted, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R^{1b} and R^{1a} are joined to form an optionally substituted heterocycle, or —OH groups or an amino acid side chain optionally substituted;
W⁴ of Formula ULM-a is an optionally substituted —NR¹-T-Aryl wherein the aryl group may be optionally substituted with an optionally substituted 5-6 membered heteroaryl or an optionally substituted aryl, an optionally substituted —NR₁-T-Heteroaryl group with an optionally substituted aryl or an optionally substituted heteroaryl, or an optionally substituted —NR₁-T-Heterocycle, where —NR₁ is covalently bonded to X² and R₁ is H or CH₃, preferably H;
n is 0 to 6, often 0, 1, 2, or 3, preferably 0 or 1; and
the [symbol] indicates the site of attachment of at least one PTM, another ULM (ULM′) or a chemical linker moiety coupling at least one PTM or a ULM′ or both to ULM.

In any aspect or embodiment described herein, T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and n is 0 to 6, often 0, 1, 2, or 3, preferably 0 or 1.

In any aspect or embodiment described herein, W⁴ of Formula ULM-a is

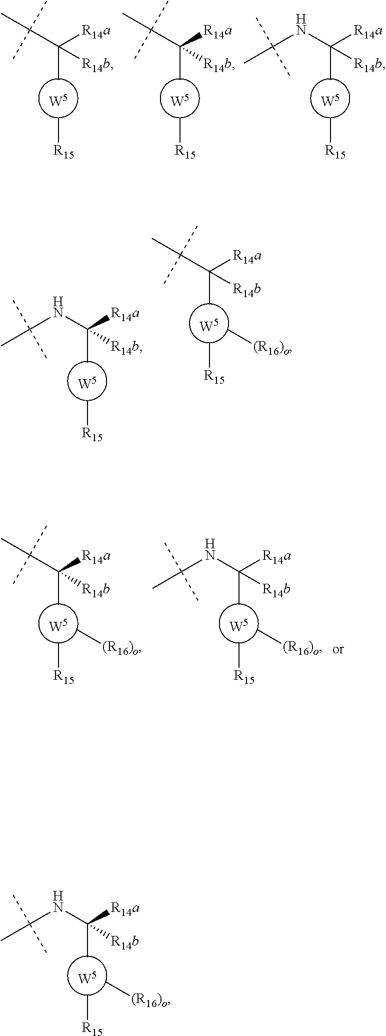

wherein:
W⁵ is optionally substituted (e.g., W⁵ is an optionally substituted phenyl, an optionally substituted napthyl, or an optionally substituted 5-10 membered heteroaryl)(e.g., W⁵ is optionally substituted with one or more [such as 1, 2, 3, 4, or 5] halo, CN, optionally substituted alkyl, optionally substituted haloalkyl, optionally substituted alkoxy, hydroxy, or optionally substituted haloalkoxy),
R₁₄ₐ, R_{14b}, are each independently selected from the group of H, haloalkyl (e.g., fluoroalkyl), optionally substituted alkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
is an integer from 0-4 (e.g., 0, 1, 2, 3, or 4); and
R₁₆ is independently selected from the group of halo, optionally substituted alkyl, optionally substituted haloalkyl, hydroxy, or optionally substituted haloalkoxy.

In any aspect or embodiment described herein, W⁵ of Formula ULM-a is selected from the group of an optionally substituted phenyl, an optionally substituted napthyl, or an optionally substituted 5-10 membered heteroaryl (e.g., W⁵ is optionally substituted with one or more [such as 1, 2, 3, 4, or 5] halo, CN, optionally substituted alkyl, optionally substituted haloalkyl, optionally substituted alkoxy, hydroxy, or optionally substituted haloalkoxy),

R₁₅ of Formula ULM-a is selected from the group of H, halogen, CN, OH, NO₂, N R₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy; optionally substituted aryl, optionally substituted heteroaryl, optionally substituted cycloalkyl, or optionally substituted cycloheteroalkyl;

In any aspect or embodiment described herein, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In any aspect or embodiment described herein, ULM-a, is optionally substituted by 0-3 R^{P} groups in the pyrrolidine moiety. Each R^{P} is independently H, halo, —OH, C1-3alkyl, C═O.

In any aspect or embodiment described herein, the W³, W⁴ of Formula ULM-a can independently be covalently coupled to a linker which is attached one or more PTM groups. and wherein the [symbol] indicates the site of attachment of at least one PTM, another ULM (ULM′) or a chemical linker moiety coupling at least one PTM or a ULM′ or both to ULM.

In any aspect or embodiment described herein, ULM is VHL and is represented by the structure:

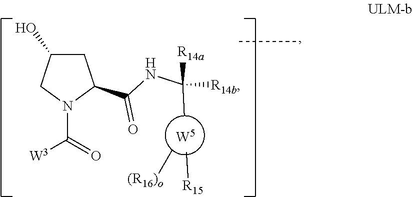

wherein:
W³ of Formula ULM-b is selected from the group of an optionally substituted aryl, optionally substituted heteroaryl, or

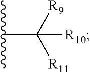

R₉ and R₁₀ of Formula ULM-b are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ of Formula ULM-b is selected from the group of an optionally substituted heterocyclyl, optionally substituted alkoxy, optionally substituted heteroaryl, optionally substituted aryl,

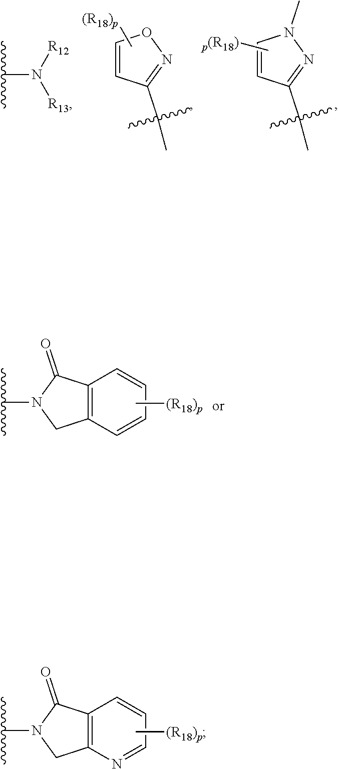

R₁₂ of Formula ULM-b is selected from the group of H or optionally substituted alkyl;
R₁₃ of Formula ULM-b is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b} of Formula ULM-b, are each independently selected from the group of H, haloalkyl (e.g. fluoroalkyl), optionally substituted alkyl, optionally substitute alkoxy, aminomethyl, alkylaminomethyl, alkoxymethyl, optionally substituted hydroxyl alkyl, optionally substituted alkylamine, optionally substituted heteroalkyl, optionally substituted alkyl-heterocycloalkyl, optionally substituted alkoxy-heterocycloalkyl, CONR₂₇ₐR_{27b}, CH₂NHCOR₂₆, or (CH₂)N(CH₃)COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalkyl, spirocycloalkyl or spiroheterocyclyl, wherein the spiroheterocyclyl is not epoxide or aziridine;
W⁵ of Formula ULM-b is selected from the group of an optionally substituted phenyl or an optionally substituted 5-10 membered heteroaryl (e.g., W⁵ is optionally substituted with one or more [such as 1, 2, 3, 4, or 5] halo, CN, optionally substituted alkyl, optionally substituted haloalkyl, optionally substituted alkoxy, hydroxy, or optionally substituted haloalkoxy),
R₁₅ of Formula ULM-b is selected from the group of H, halogen, CN, OH, NO₂, N R₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;
each R₁₆ of Formula ULM-b is independently selected from the group of H, CN, halo, optionally substituted alkyl, optionally substituted haloalkyl, hydroxy, or optionally substituted haloalkoxy;
o of Formula ULM-b is 0, 1, 2, 3, or 4;
R₁₈ of Formula ULM-b is independently selected from the group of H, halo, optionally substituted alkoxy, cyano, optionally substituted alkyl, haloalkyl, haloalkoxy or a linker;
p of Formula ULM-b is 0, 1, 2, 3, or 4, and
the [symbol] indicates the site of attachment of at least one PTM, another ULM (ULM′) or a chemical linker moiety coupling at least one PTM or a ULM′ or both to ULM.

In any aspect or embodiment described herein, R₁₅ of Formula ULM-b is selected from the group of H, halogen, CN, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl, wherein each R₂₆ is independently selected from H, optionally substituted alkyl or NR₂₇ₐR_{27b}; and each R₂₇ₐ and R_{27b} is independently H, optionally substituted alkyl, or R₂₇ₐ and R_{27b} together with the nitrogen atom to which they are attached form a 4-6 membered heterocyclyl.

In any aspect or embodiment described herein, R₁₅ of Formula ULM-b is

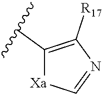

wherein R₁₇ is H, halo, optionally substituted C₃₋₆ cycloalkyl, optionally substituted C₁₋₆ alkyl, optionally substituted C₁₋₆ alkenyl, and C₁₋₆ haloalkyl; and Xa is S or O.

In any aspect or embodiment described herein, R₁₇ of Formula ULM-b is selected from the group methyl, ethyl, isopropyl, and cyclopropyl.

In any aspect or embodiment described herein, R₁₅ of Formula ULM-b is selected from the group consisting of:

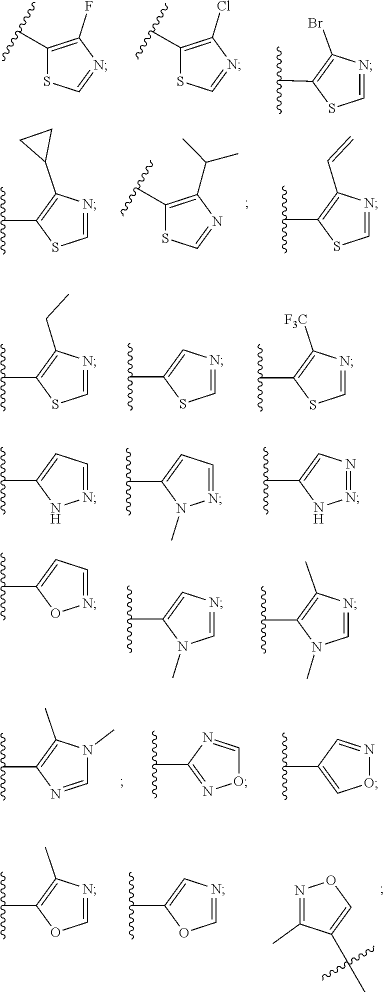

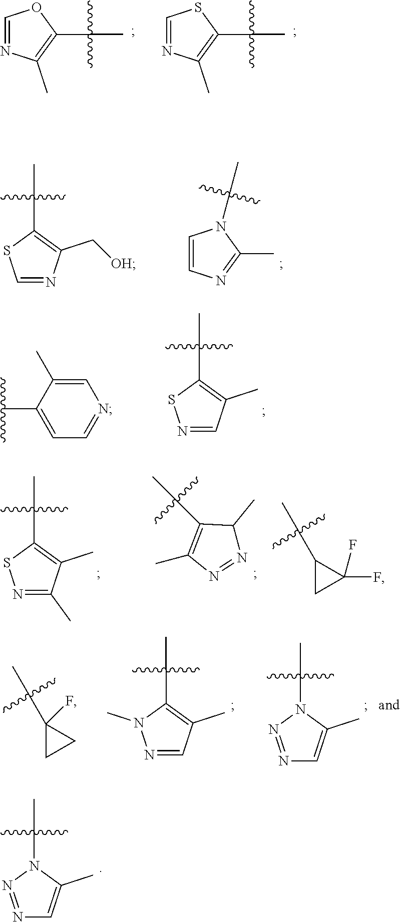

In any aspect or embodiment described herein, R₁₁ of Formula ULM-b is selected from the group consisting of:

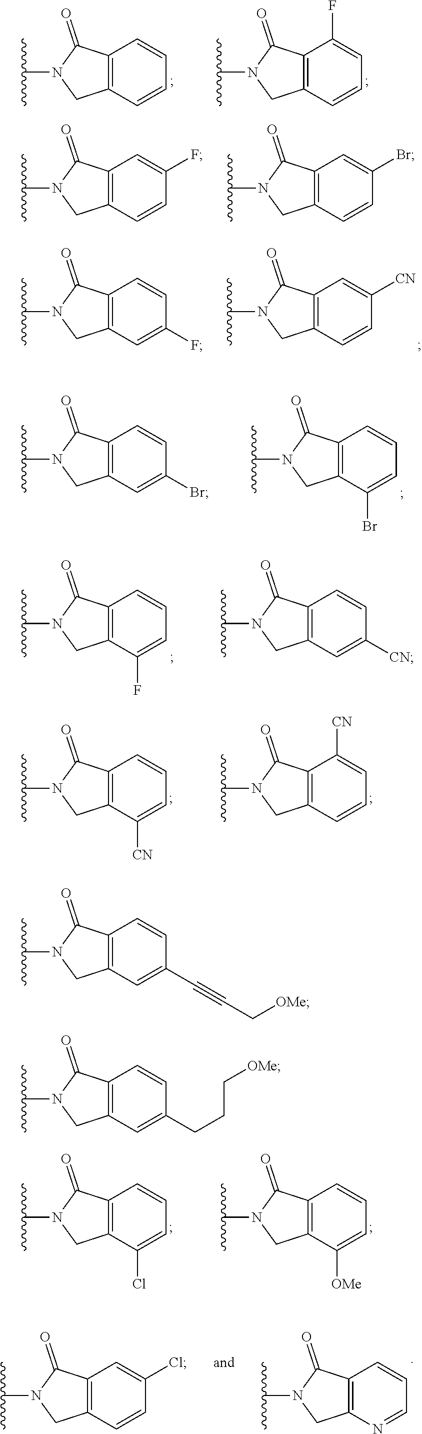

In any aspect or embodiment described herein, R₁₄ₐ, R_{14b} of Formula ULM-b, are each independently selected from the group of H, optionally substituted haloalkyl, optionally substituted alkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered cycloalkyl, heterocycloalkyl, spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl, wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted; R₂₆ and R₂₇ are as described above.

In any aspect or embodiment described herein, R₁₅ of Formula ULM-b is selected from H, halogen, CN, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl (e.g. optionally substituted fluoroalkyl), optionally substituted haloalkoxy, optionally substituted aryl, optionally substituted heteroaryl, optionally substituted cycloalkyl, or optionally substituted heterocyclyl wherein optional substitution of the said aryl, heteroaryl, cycloalkyl and heterocycloalkyl includes CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, CH₂NCH₃COR₂₆ or

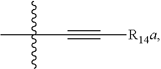

wherein R₂₆, R₂₇, R₃₀ and R₁₄ₐ are as described above.

In any aspect or embodiment described herein, R₁₄ₐ, R_{14b} of Formula ULM-b, are each independently selected from the group of H, optionally substituted haloalkyl, optionally substituted alkyl, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl, wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted;

R₁₅ of Formula ULM-b is selected from H, halogen, CN, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl wherein optional substitution of the said aryl, heteroaryl, cycloalkyl and heterocycloalkyl includes CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, CH₂NCH₃COR₂₆ or

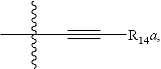

wherein R₂₆, R₂₇, R₃₀ and R₁₄ₐ are as described above.

In any aspect or embodiment described herein, ULM has a chemical structure selected from the group of:

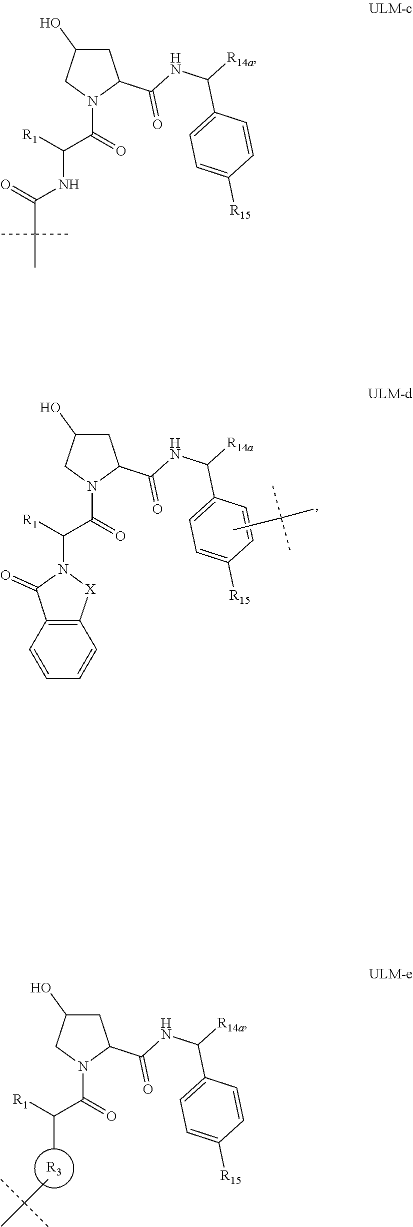

wherein:
R₁ of Formulas ULM-c, ULM-d, and ULM-e is H, ethyl, isopropyl, tert-butyl, sec-butyl, cyclopropyl, cyclobutyl, cyclopentyl, or cyclohexyl; optionally substituted alkyl, optionally substituted cycloalkyl, optionally substituted hydroxyalkyl, optionally substituted heteroaryl, or haloalkyl;
R₁₄ₐ of Formulas ULM-c, ULM-d, and ULM-e is H, haloalkyl, optionally substituted alkyl, methyl, fluoromethyl, hydroxymethyl, ethyl, isopropyl, or cyclopropyl;
R₁₅ of Formulas ULM-c, ULM-d, and ULM-e is selected from the group consisting of H, halogen, CN, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted cycloheteroalkyl;
X of Formulas ULM-c, ULM-d, and ULM-e is C, CH₂, or C═O
R₃ of Formulas ULM-c, ULM-d, and ULM-e is absent or an optionally substituted 5 or 6 membered heteroaryl; and
the [symbol] indicates the site of attachment of at least one PTM, another ULM (ULM′) or a chemical linker moiety coupling at least one PTM or a ULM′ or both to ULM.

In any aspect or embodiment described herein, ULM comprises a group according to the chemical structure:

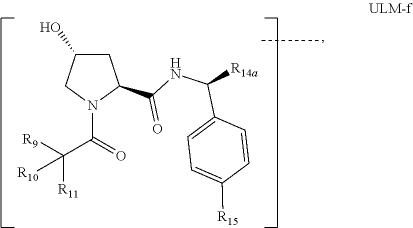

wherein:
R₁₄ₐ of Formula ULM-f is H, haloalkyl, optionally substituted alkyl, methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

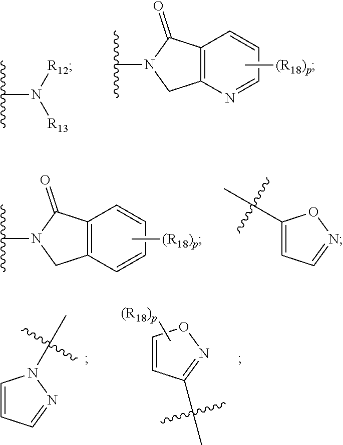

or optionally substituted heteroaryl;
p of Formula ULM-f is 0, 1, 2, 3, or 4;
each R₁₈ of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ of Formula ULM-f is selected from the group consisting of H, halogen, Cl, CN, OH, NO₂, optionally substituted haloalkyl, optionally substituted heteroaryl, optionally substituted aryl;

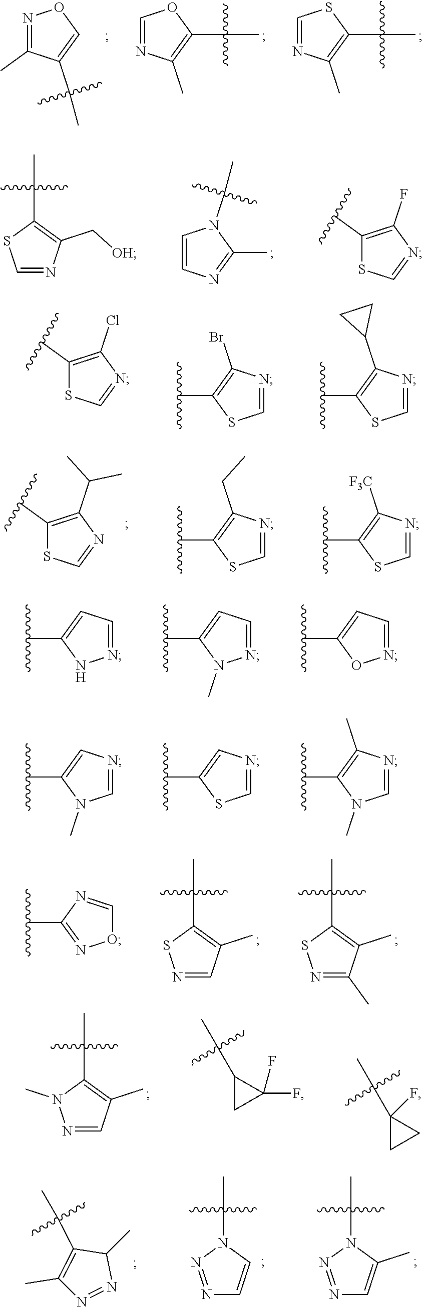

and
the [symbol] of Formula ULM-f indicates the site of attachment of at least one PTM, another ULM (ULM′) or a chemical linker moiety coupling at least one PTM or a ULM′ or both to ULM.

In any aspect or embodiment described herein, the VLM is covalently joined to a PTM, or a chemical linker group (L) via an R group (such as, R^{P}, R¹, R^{1a}, R^{1b}, R^{Y3}, R^{Y4}, R₉, R₁₀, R₁₁, R₁₂, R₁₃, R₁₄ₐ, R_{14b}, R₁₅, R₁₆, R₁₇, R₁₈, R₂₆, R₂₇ₐ, R_{27b}, R₃₀, R₃₃), W³, W⁴, W⁵, X, X¹, X², X³, or T.

In any aspect or embodiment described herein, the VLM is covalently joined to a PTM, or a chemical linker group (L) via R^{P}, R¹, R^{1a}, R^{1b}, R^{Y3}, R^{Y4}, R₉, R₁₀, R₁₁, R₁₂, R₁₃, R₁₄ₐ, R_{14b}, R₁₅, R₁₆, R₁₇, R₁₈, R₂₆, R₂₇ₐ, R_{27b}, R₃₀, R₃₃, W³, W⁴, W⁵, X, X¹, X², X³, or T.

In any aspect or embodiment described herein, the R^{P}, R¹, R^{1a}, R^{1b}, R^{Y3}, R^{Y4}, R₉, R₁₀, R₁₁, R₁₂, R₁₃, R₁₄ₐ, R_{14b}, R₁₅, R₁₆, R₁₇, R₁₈, R₂₆, R₂₇ₐ, R_{27b}, R₃₀, R₃₃, W³, W⁴, X, X¹, X², X³, or T can independently be covalently coupled to a linker and/or a linker to which is attached to one or more PTM, ULM, and VLM group.

In any aspect or embodiment described herein, the ULM is selected from the following structures:

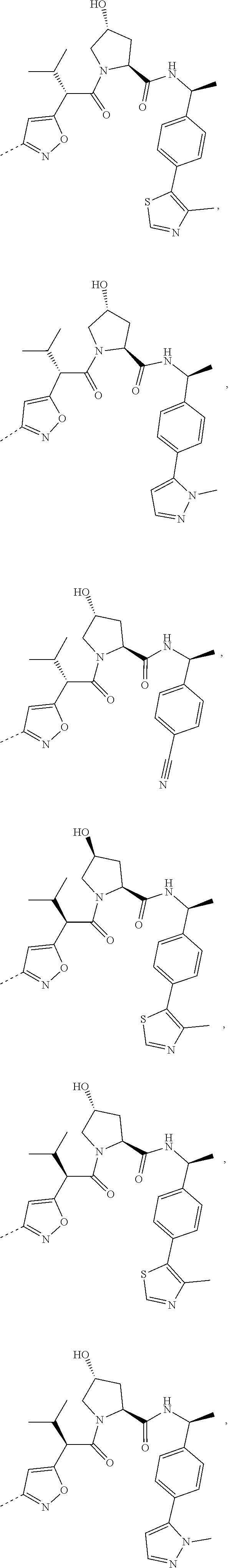

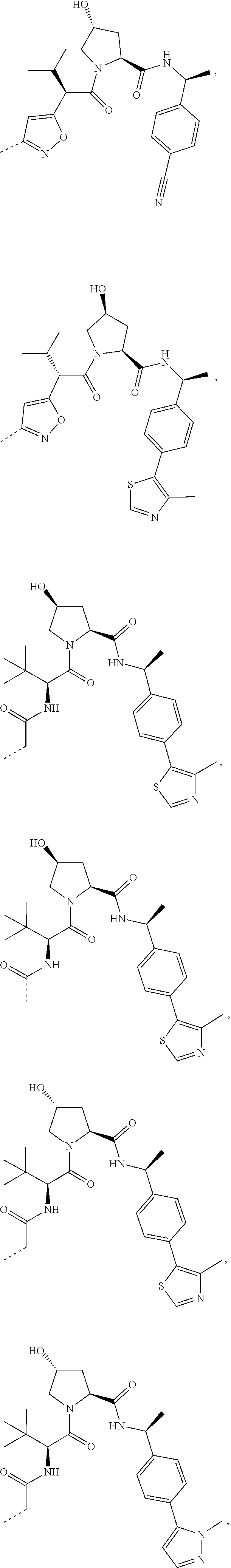

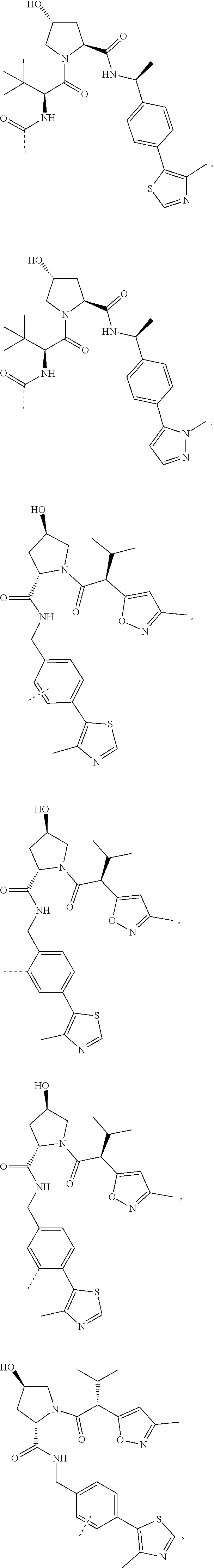

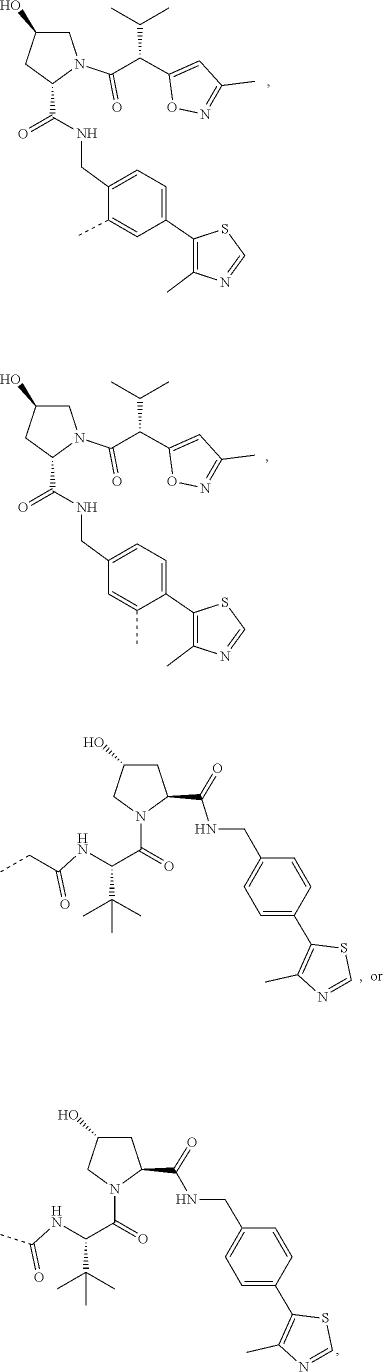

wherein the [symbol] indicates the site of attachment of at least one PTM, another ULM (ULM′) or a chemical linker moiety coupling at least one PTM or a ULM′ or both to ULM.

In any aspect or embodiment described herein, the ULM is selected from the following structures:

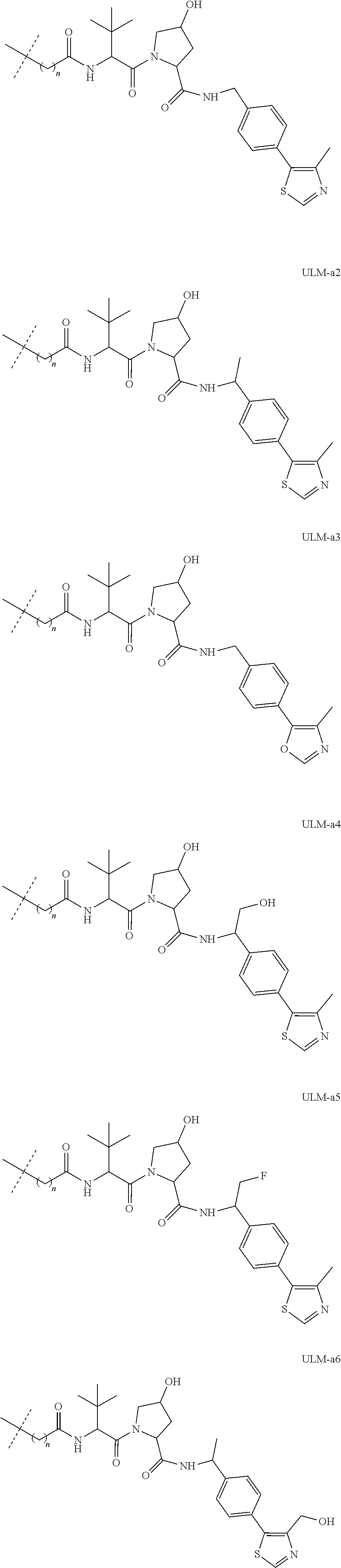

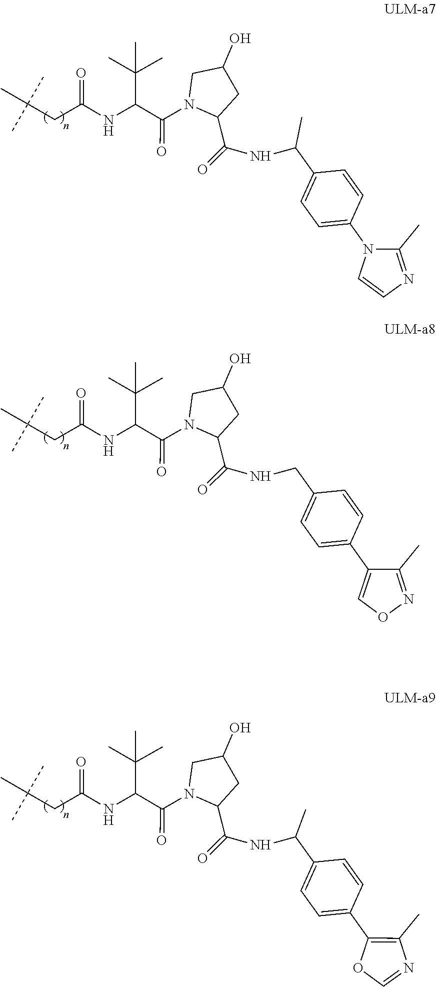

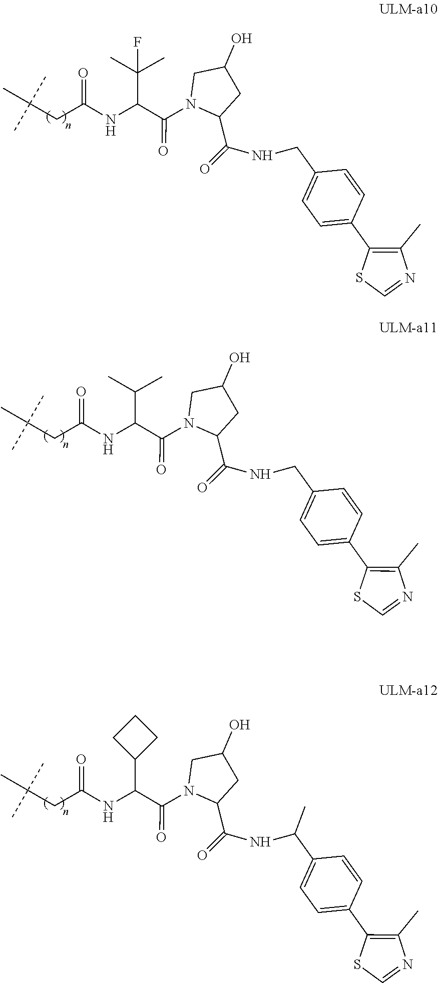

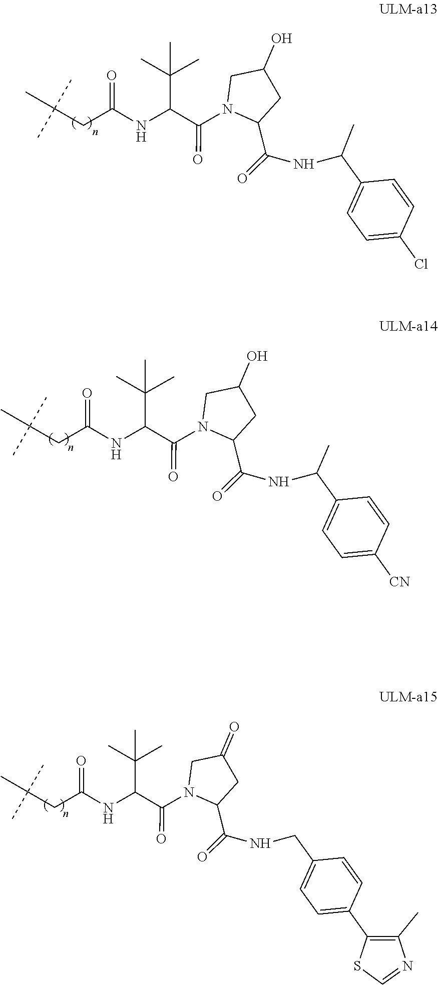

wherein n is 0 or 1 and the [symbol] indicates the site of attachment of at least one PTM, another ULM (ULM′) or a chemical linker moiety coupling at least one PTM or a ULM′ or both to ULM.

In any aspect or embodiment described herein, the ULM is selected from the following structures:

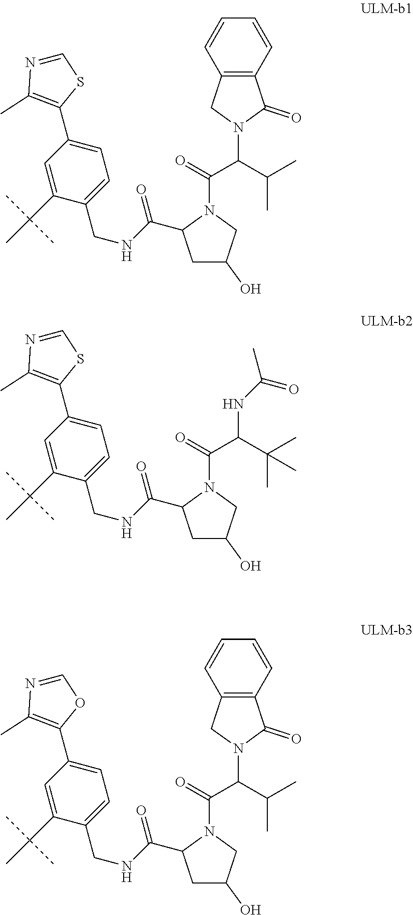

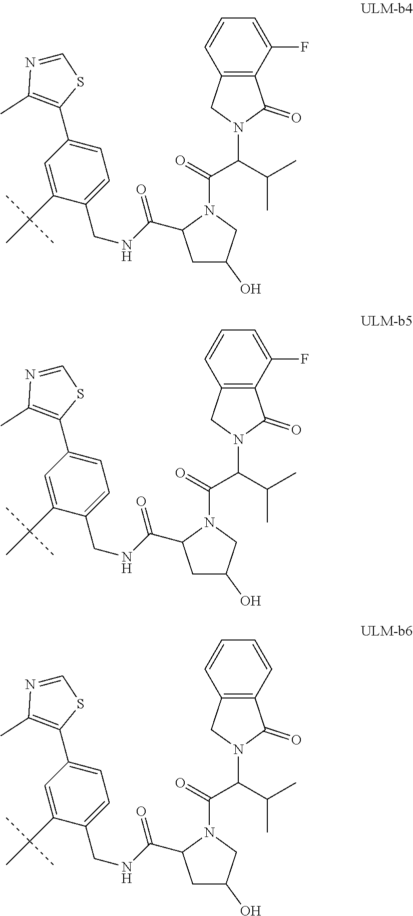

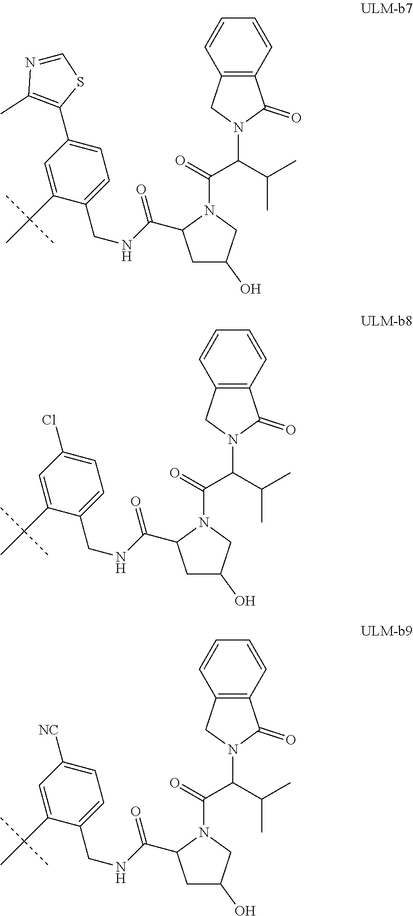

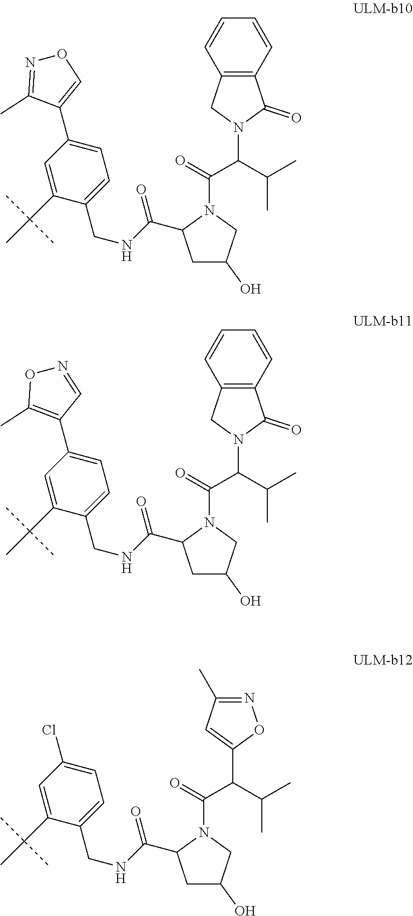

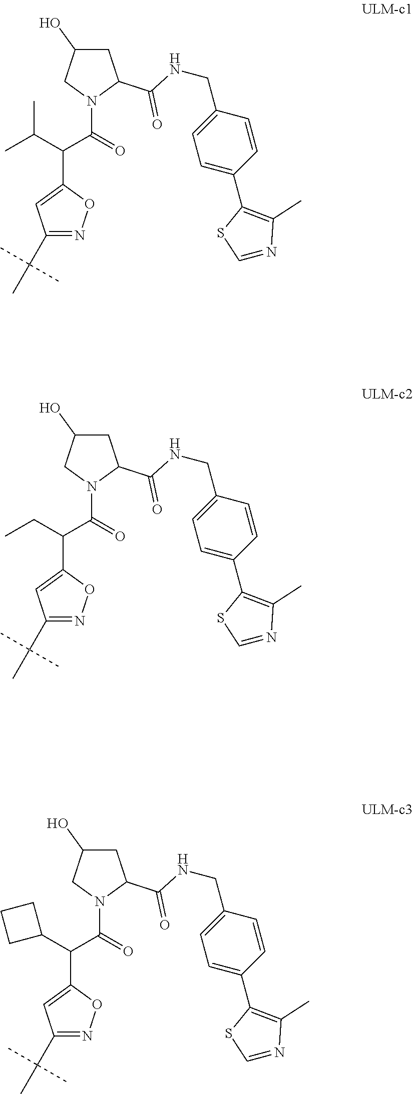

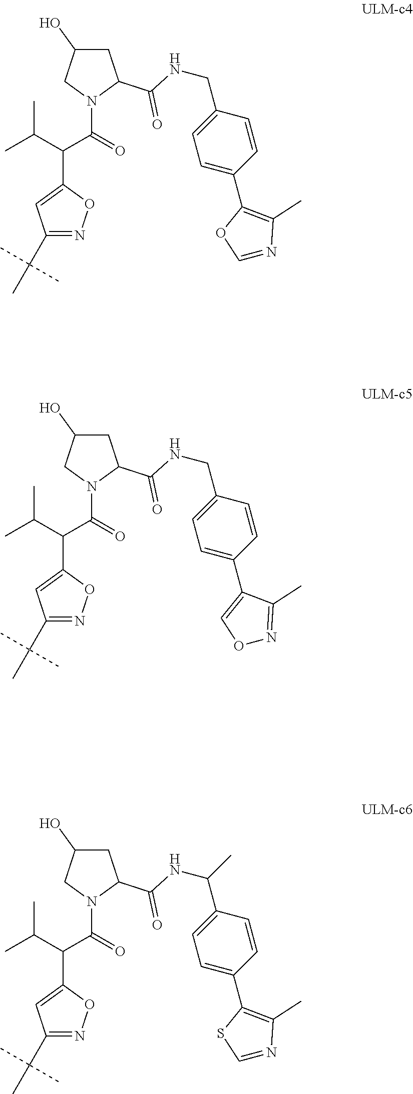

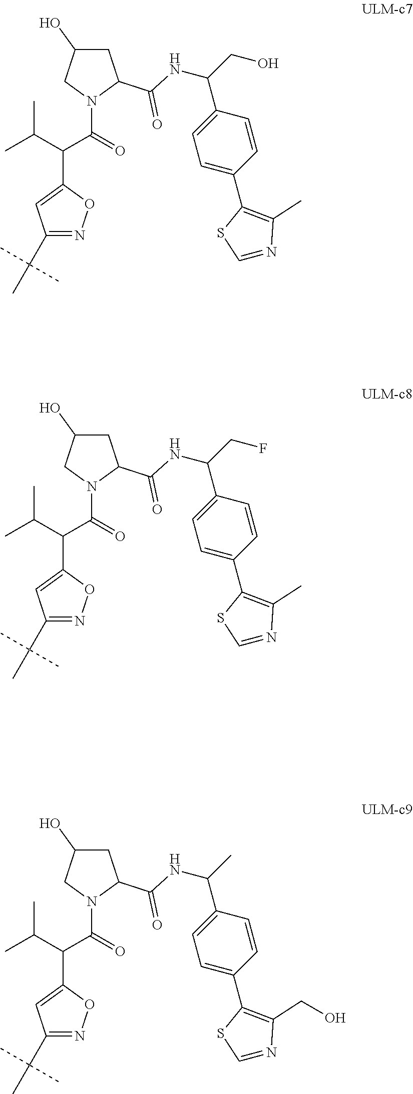

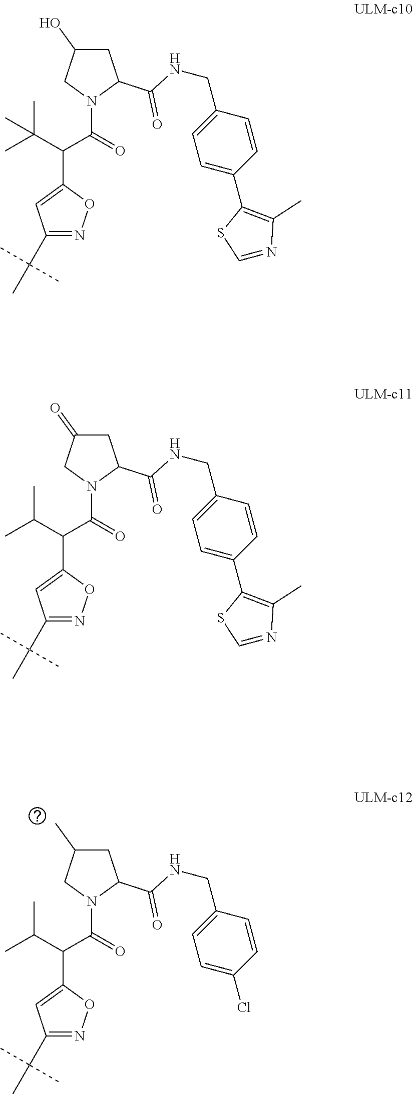

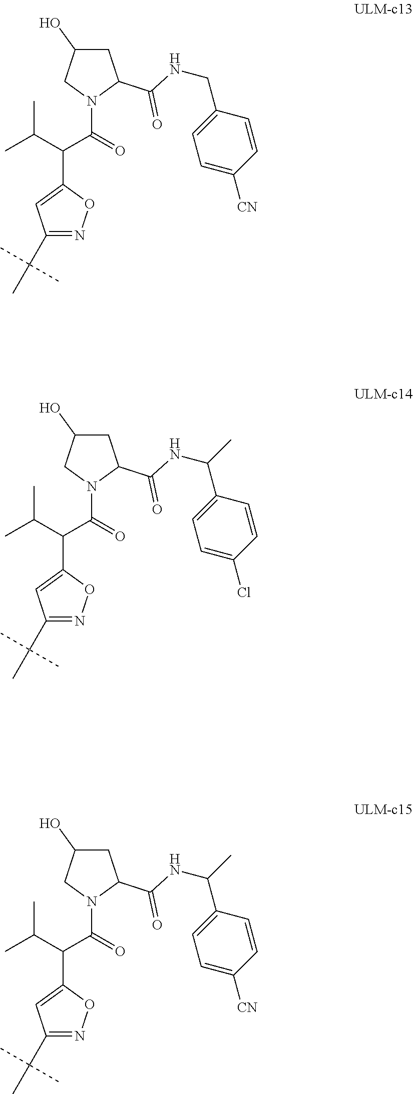

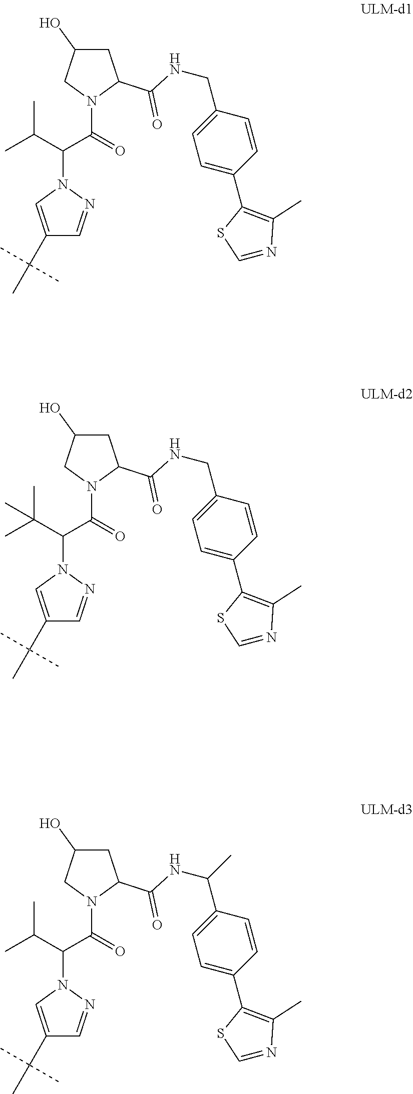

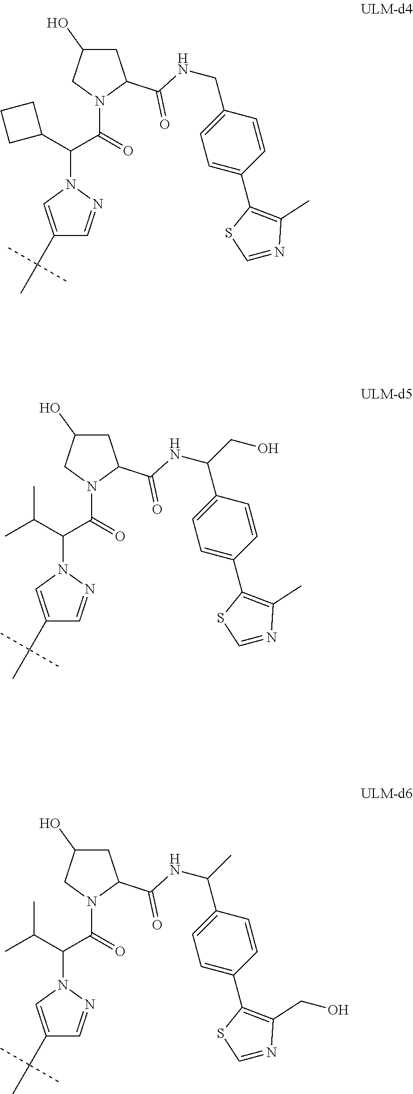

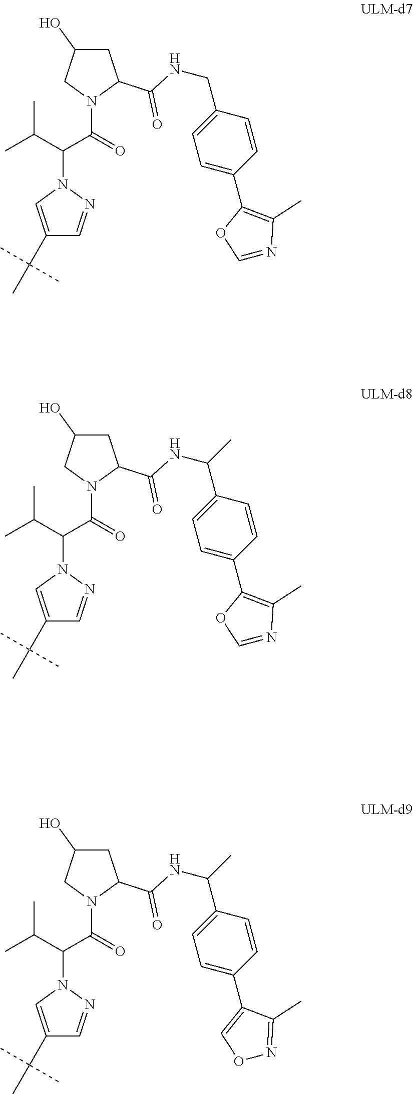

wherein, the phenyl ring in ULM-a1 through ULM-a15, ULM-b1 through ULM-b12, ULM-c1 through ULM-c15 and ULM-d1 through ULM-d9 is optionally substituted with fluorine, lower alkyl and alkoxy groups, and wherein the [symbol] indicates the site of attachment of at least one PTM, another ULM (ULM′) or a chemical linker moiety coupling at least one PTM or a ULM′ or both to ULM-a.

In any aspect or embodiment described herein, the phenyl ring in ULM-a1 through ULM-a15, ULM-b1 through ULM-b12, ULM-c1 through ULM-c15 and ULM-d1 through ULM-d9 can be functionalized as the ester to make it a part of the prodrug.

In any aspect or embodiment described herein, the hydroxyl group on the pyrrolidine ring of ULM-a1 through ULM-a15, ULM-b1 through ULM-b12, ULM-c1 through ULM-c15 and ULM-d1 through ULM-d9, respectively, comprises an ester-linked prodrug moiety.

In any aspect or embodiment described herein, the ULM and where present, ULM′, are each independently a group according to the chemical structure:

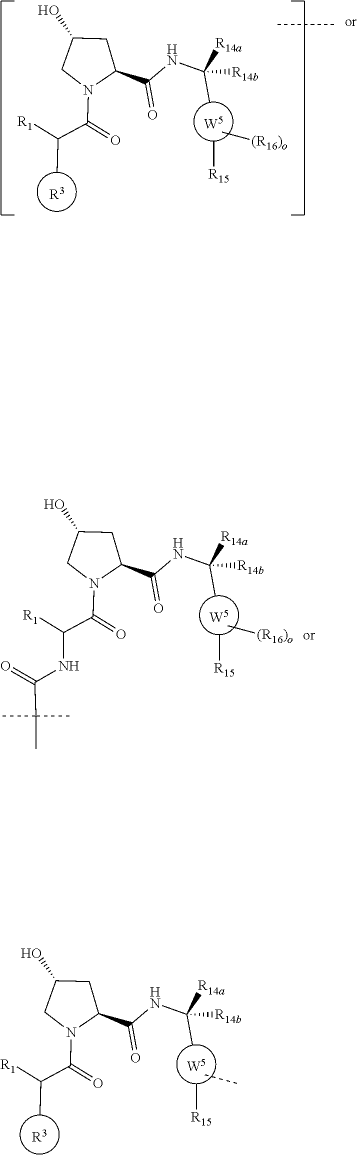

or a pharmaceutically acceptable salt thereof, wherein:
R₁ is H, optionally substituted alkyl or optionally substituted cycloalkyl;
R₃ is an optionally substituted 5-6 membered heteroaryl;
W⁵ is optionally substituted phenyl, optionally substituted napthyl or optionally substituted pyridinyl;
one of R₁₄ₐ and R_{14b} is H, optionally substituted alkyl, optionally substituted haloalkyl (e.g., fluoroalkyl), optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalkyl, spirocycloalkyl or spiroheterocyclyl, wherein the spiroheterocyclyl is not epoxide or aziridine;
R₁₅ is CN, optionally substituted fluoroalkyl,

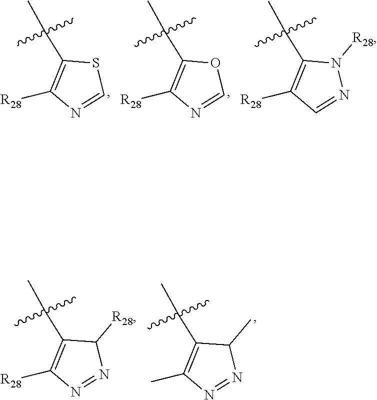

optionally substituted

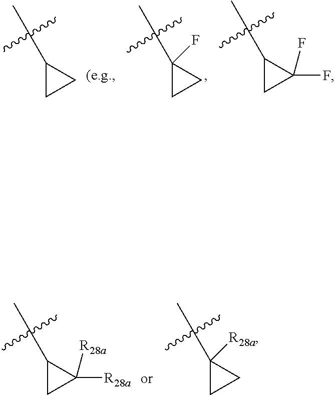

wherein R₂₈ₐ is halo, optionally substituted alkyl or fluoroalkyl), or

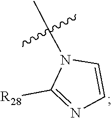

each R₁₆ is independently selected from halo, CN, optionally substituted alkyl, optionally substituted haloalkyl, hydroxy, or haloalkoxy;
each R₂₆ is independently H, optionally substituted alkyl or NR₂₇ₐR_{27b};
each R₂₇ₐ and R_{27b} is independently H, optionally substituted alkyl, or R₂₇ₐ and R_{27b} together with the nitrogen atom to which they are attached form a 4-6 membered heterocyclyl; R₂₈ is H, optionally substituted alkyl, optionally substituted alkoxy, optionally substituted heteroalkyl, optionally substituted alkylamine, optionally substituted hydroxyalkyl, amine, optionally substituted alkynyl, or optionally substituted cycloalkyl;
is 0, 1 or 2; and
the [symbol] or [symbol] indicates the site of attachment of at least one PTM, another ULM (ULM′) or a chemical linker moiety coupling at least one PTM or a ULM′ or both to ULM.

In any of the aspects or embodiments described herein, the ULM is of the formula:

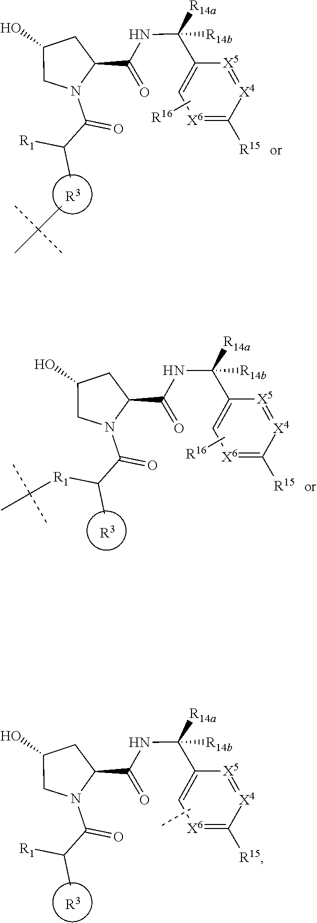

wherein:
each of X⁴, X⁵, and X⁶ is selected from CH and N, wherein no more than 2 are N;
R¹ is C1-6 alkyl;
R₃ is an optionally substituted 5-6 membered heteroaryl;
one of R^{14a} and R^{14b} is H, optionally substituted alkyl, optionally substituted haloalkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶; and the other of R^{14a} and R^{14b} is H; or R^{14a} and R^{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
each R₂₇ₐ and R_{27b} is independently H or C₁₋₆ alkyl;
q is 1, 2, 3 or 4;
R¹⁵ is,

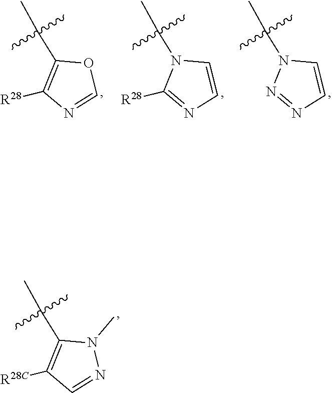

or CN;
R²⁸ is H, methyl, CH₂N(Me)₂, CH₂OH, CH₂O(C₁₋₄ alkyl), CH₂NHC(O)C₁₋₄ alkyl, NH₂,

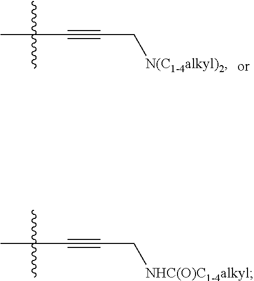

R^{28C} is H, methyl, fluoro, or chloro;
R¹⁶ is H, C₁₋₄ alkyl, fluoro, chloro, CN, or C₁₋₄ alkoxy; and
the [symbol] or [symbol] indicates the site of attachment of at least one PTM, another ULM (ULM′) or a chemical linker moiety coupling at least one PTM or a ULM′ or both to ULM.

In any aspect or embodiment described herein, R^{14a} and R^{14b} are selected from: H, C₁₋₄ alkyl, C₁₋₄ cycloalkyl, C₁₋₄ haloalkyl, C₁₋₄ hydroxyalkyl, C₁₋₄ alkyloxyalkyl, C₁₋₄ alkyl-NR₂₇ₐR_{27b} and CONR₂₇ₐR_{27b}.

In any aspect or embodiment described herein, at least one of R^{14a} and R^{14b} is H (e.g., both R^{14a} and R^{14b} are H).

In any aspect or embodiment described herein, at least one of R^{14a} and R^{14b} is optionally substituted alkyl, optionally substituted haloalkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶. Alternatively, in any aspect or embodiment described herein, one of R^{14a} and R^{14b} is optionally substituted alkyl, optionally substituted haloalkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶; and the other of R^{m}a and R^{14b} is H.

In any aspect or embodiment described herein, R^{14a} and R^{14b} together with the carbon atom to which they are attached form

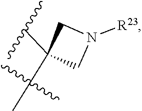

wherein R²³ is selected from H, C₁₋₄ alkyl, —C(O)C₁₋₄ alkyl.

In any aspect or embodiment described herein, ULM and where present, ULM′, are each independently a group according to the chemical structure:

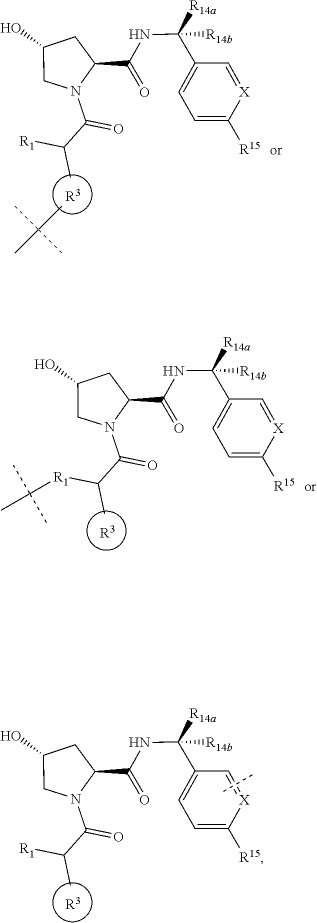

or a pharmaceutically acceptable salt thereof, wherein:
X is CH or N;
R₁ is H, optionally substituted alkyl or optionally substituted cycloalkyl;
R₃ is an optionally substituted 5-6 membered heteroaryl;
one of R₁₄ₐ and R_{14b} is H, optionally substituted alkyl, optionally substituted haloalkyl (e.g., fluoroalkyl), optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalkyl, spirocycloalkyl or spiroheterocyclyl, wherein the spiroheterocyclyl is not epoxide or aziridine;
R₁₅ is CN, optionally substituted fluoroalkyl,

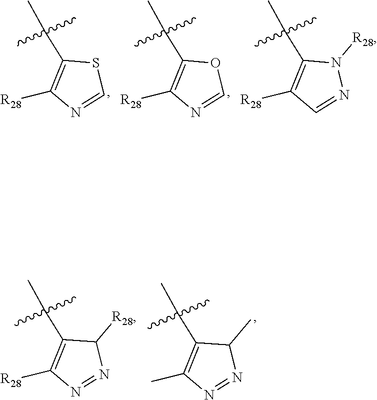

optionally substituted

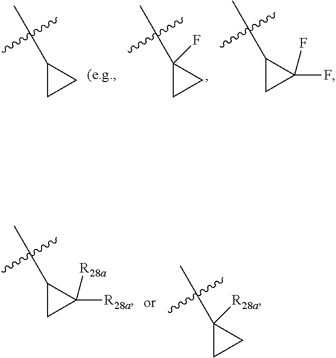

wherein R₂₈ₐ is halo, optionally substituted alkyl or fluoroalkyl), or

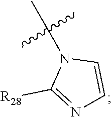

each R₂₆ is independently H, optionally substituted alkyl or NR₂₇ₐR_{27b};
each R₂₇ₐ and R_{27b} is independently H, optionally substituted alkyl, or R₂₇ₐ and R_{27b} together with the nitrogen atom to which they are attached form a 4-6 membered heterocyclyl;
R₂₈ is H, optionally substituted alkyl, optionally substituted alkoxy, optionally substituted heteroalkyl, optionally substituted alkylamine, optionally substituted hydroxyalkyl, amine, optionally substituted alkynyl, or optionally substituted cycloalkyl;
the [symbol] or [symbol] indicates the site of attachment of at least one PTM, another ULM (ULM′) or a chemical linker moiety coupling at least one PTM or a ULM′ or both to ULM.

In any of the aspects or embodiments described herein, R₁ is C₁₋₆ alkyl.

In any of the aspects or embodiments described herein, one of R₁₄ₐ and R_{14b} is H, C₁₋₆ alkyl, C₁₋₆ haloalkyl, optionally substitute C₁₋₄ alkylamine, C₁₋₆ alkoxy, (CH₂)_{q}C₁₋₆ alkoxy, (CH₂)_{q}C₁₋₆ alkoxy-C3-C₇ heterocycloalkyl, (CH₂), PH, (CH₂)_{q}NR₂₇ₐR_{27b}, (CH₂)_{q}NHCOC₁₋₆ alkyl, C₃₋₆ cycloalkyl, or NR₂₇ₐR_{27b}; each R₂₆ is independently H, C₁₋₆ alkyl or NR₂₇ₐR_{27b}; each R₂₇ₐ and R_{27b} is independently H or C₁₋₆ alkyl; and q is 1, 2, 3 or 4.

In any of the aspects or embodiments described herein, one of R₁₄ₐ and R_{14b} is H, C₁₋₄ alkyl, C14 haloalkyl, C₁₋₄ alkoxy, optionally substituted C₁₋₄ alkylamine, (CH₂)_{q}C₁₋₆ alkoxy, (CH₂)_{q}C₁₋₆ alkoxy-C3-C₇ heterocycloalkyl, (CH₂), PH, (CH₂)_{q}NR₂₇ₐR_{27b}, (CH₂)_{q}NHCOC₁₋₆ alkyl, C₃₋₆ cycloalkyl, or NR₂₇ₐR_{27b}; each R₂₆ is independently H, C₁₋₄ alkyl or NR₂₇ₐR_{27b}; each R₂₇ₐ and R_{27b} is independently H or C₁₋₄ alkyl; and q is 1 or 2.

In any of the aspects or embodiments described herein, R₂₈ is C₁₋₆ alkyl, C₃₋₆ cycloalkyl, C₁₋₆ haloalkyl, (CH₂)_{q}OC₁₋₆ alkyl, (CH₂)_{q}OH, (CH₂)_{q}NR₂₇ₐR_{27b}, (CH₂)_{q}NHCOC₁₋₆ alkyl, or

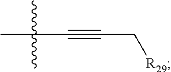

R₂₉ is H, C₁₋₆ alkyl, NR₂₇ₐR_{27b} or _{q}NHCOC₁₋₆ alkyl; and wherein q is 1 or 2.

In any of the aspects or embodiments described herein, R³ is isoxazolyl, 4-chloroisoxazolyl, 4-fluoroisoxazolyl, or pyrazolyl. In any of the aspects or embodiments described herein, X is CH.

In any aspect or embodiment described herein, the ULM is according to the formula:

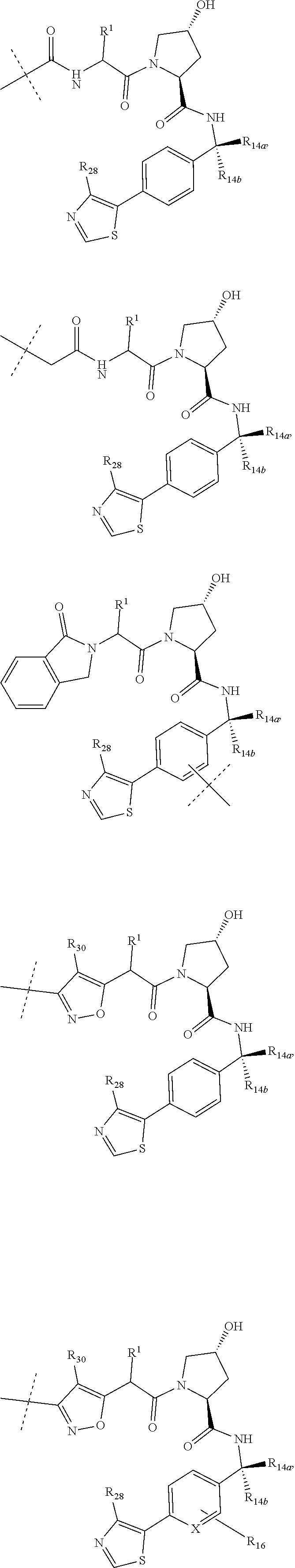

or a pharmaceutically acceptable salt thereof,
wherein:
R₁, R₁₄ₐ and R_{14b} are as described herein;
X is CH or N;
R₃₀ is H, F or Cl;
R¹⁶ is H, C₁₋₄ alkyl, fluoro, chloro, CN, or C₁₋₄ alkoxy;
R₂₈ is H, methyl, CH₂N(Me)₂, CH₂OH, CH₂O(C₁₋₄ alkyl), CH₂NHC(O)C₁₋₄ alkyl, NH₂,

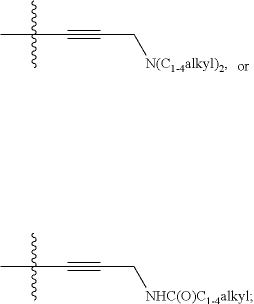

and
the [symbol] indicates the site of attachment of at least one PTM, another ULM (ULM′) or a chemical linker moiety coupling at least one PTM or a ULM′ or both to ULM.

In any of the aspects or embodiments described herein, the ULM is according to the formula:

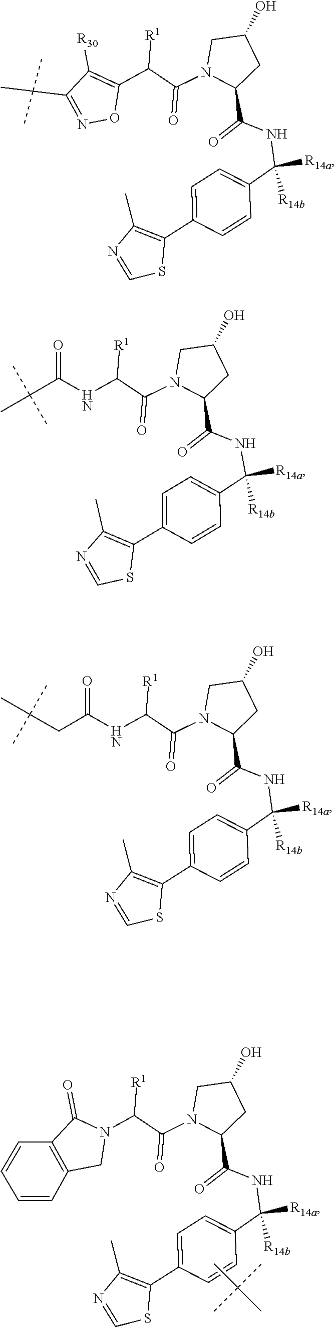

or a pharmaceutically acceptable salt thereof,

wherein:
each of R₁, R₁₄ₐ, R_{14b} are as described herein;
R₃₀ is H, F or Cl; and
the [symbol] indicates the site of attachment of at least one PTM, another ULM (ULM′) or a chemical linker moiety coupling at least one PTM or a ULM′ or both to ULM.

In any aspect or embodiment described herein, the VLM is covalently joined to a PTM, or a chemical linker group (L) via an R group (such as, R¹, R³, R₁₄ₐ, R_{14b}, R₁₅, R₁₆, R²³, R²⁶, R₂₇ₐ, R_{27b}, R₂₈, R₂₈ₐ, R_{28C}, R₂₉, R₃₀), X, X⁴, X⁵, or X⁶.

In any aspect or embodiment described herein, the VLM is covalently joined to a PTM, or a chemical linker group (L) via R¹, R³, R₁₄ₐ, R_{14b}, R₁₅, R₁₆, R²³, R²⁶, R₂₇ₐ, R_{27b}, R₂₈, R₂₈ₐ, , R_{28C}, R₂₉, R₃₀, X, X⁴, X⁵, or X⁶.

In any aspect or embodiment described herein, the R¹, R³, R₁₄ₐ, R_{14b}, R₁₅, R₁₆, R²³, R²⁶, R₂₇ₐ, R_{27b}, R₂₈, R₂₈ₐ, R_{28C}, R₂₉, R₃₀, X, X⁴, X⁵, or X⁶ can independently be covalently coupled to a linker and/or a linker to which is attached to one or more PTM, ULM, and VLM group.

In any of the aspects or embodiments described herein, the ULM (or when present, ULM′) as described herein may be a pharmaceutically acceptable salt, enantiomer, diastereomer, solvate or polymorph thereof. In addition, in any of the aspects or embodiments described herein, the ULM (or when present, ULM′) as described herein may be coupled to a PTM directly via a bond or by a chemical linker.

### Exemplary Linkers

In any aspect or embodiment described herein, the compounds as described herein include a PTM chemically linked to a ULM (e.g., VLM) via a chemical linker (L). In certain embodiments, the linker group L comprises one or more covalently connected structural units (e.g., -A^{L}₁ . . . (A^{L})_{q}- or -(A^{L})_{q}-), wherein A^{L}₁ is a group coupled to PTM, and (A^{L})_{q} is a group coupled to ULM.

In any aspect or embodiment described herein, the linker (L) to a ULM (e.g., VLM) connection is a stable L-ULM connection. For example, in any aspect or embodiment described herein, when a linker (L) and a ULM are connected via a heteroatom (e.g., N, O, S), any additional heteroatom, if present, is separated by at least a carbon atom (e.g., —CH₂—), such as with an acetal or aminal group. By way of further example, in any aspect or embodiment described herein, when a linker (L) and a ULM are connected via a heteroatom, the heteroatom is not part of an ester.

In any aspect or embodiment described herein, the linker group L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80), and wherein L is covalently bound to both the PTM and the ULM, and provides for binding of the PTM to the protein target and the ULM to an E3 ubiquitin ligase to effectuate target protein ubiquitination.

In any aspect or embodiment described herein, the linker group L is a bond or a chemical linker group represented by the formula -(A^{L})_{q}-, wherein A is a chemical moiety and q is an integer from 6-30 (e.g., 1, 2, 3, 4, 5, 6, 7, 8, 9, 10, 11, 12, 13, 14, 15, 16, 17, 18, 19, 20, 21, 22, 23, 24, or 25), and wherein L is covalently bound to both the PTM and the ULM, and provides for binding of the PTM to the protein target and the ULM to an E3 ubiquitin ligase in sufficient proximity to result in target protein ubiquitination.

In any aspect or embodiment described herein, the linker group L is -(A^{L})_{q}-, wherein:
(A^{L})_{q} is a group which connects a ULM (e.g., VLM), to PTM (KTM);
q of the linker is an integer greater than or equal to 1;
each A^{L} is independently selected from the group consisting of, a bond, CR^{L1}R^{L2}, O, S, SO, SO₂, N^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 1-6 R^{L1} and/or R^{L2} groups, C₅₋₁₃ spirocycloalkyl optionally substituted with 1-9 R^{L1} and/or R^{L2} groups, C₃₋₁₁heterocyclyl optionally substituted with 1-6 R^{L1} and/or R^{L2} groups, C₅₋₁₃ spiroheterocyclyl optionally substituted with 1-8 R^{L1} and/or R^{L2} groups, aryl optionally substituted with 1-6 R^{L1} and/or R^{L2} groups, heteroaryl optionally substituted with 1-6 R^{L1} and/or R^{L2} groups, where R^{L1} or R^{L2}, each independently are optionally linked to other groups to form cycloalkyl and/or heterocyclyl moiety, optionally substituted with 1-4 R^{L5} groups; and
R^{L1}, R^{L2}, R^{L3}, R^{L4} and R^{L5} are, each independently, H, halo, C₁₋₈ alkyl, OC₁₋₈ alkyl, SC₁₋₈ alkyl, NHC₁₋₈ alkyl, N(C₁₋₈alkyl)₂, C₃₋₁₁cycloalkyl, aryl, heteroaryl, C₃₋₁₁heterocyclyl, OC₃₋₈cycloalkyl, SC₃₋₈ cycloalkyl, NHC₃₋₈ cycloalkyl, N(C₃₋₈ cycloalkyl)₂, N(C₃₋₈ cycloalkyl)(C₁₋₈alkyl), OH, NH₂, SH, SO₂C₁₋₈ alkyl, P(O)(OC₁₋₈ alkyl)(C₁₋₈ alkyl), P(O)(OC₁₋₈ alkyl)₂, CC—C₁₋₈ alkyl, CCH, CH═CH(C₁₋₈ alkyl), C(C₁₋₈ alkyl)═CH(C₁₋₈ alkyl), C(C₁₋₈ alkyl)═C(C₁₋₈alkyl)₂, Si(OH)₃, Si(C₁₋₈ alkyl)₃, Si(OH)(C₁₋₈ alkyl)₂, COC₁₋₈ alkyl, CO₂H, halogen, CN, CF₃, CHF₂, CH₂F, NO₂, SF₅, SO₂NHC₁₋₈ alkyl, SO₂N(C₁₋₈ alkyl)₂, SONHC₁₋₈ alkyl, SON(C₁₋₈ alkyl)₂, CONHC₁₋₈ alkyl, CON(C₁₋₈ alkyl)₂, N(C₁₋₈ alkyl)CONH(C₁₋₈ alkyl), N(C₁₋₈alkyl)CON(C₁₋₈ alkyl)₂, NHCONH(C₁₋₈ alkyl), NHCON(C₁₋₈ alkyl)₂, NHCONH₂, N(C₁₋₈alkyl)SO₂NH(C₁₋₈ alkyl), N(C₁₋₈ alkyl) SO₂N(C₁₋₈ alkyl)₂, NH SO₂NH(C₁₋₈ alkyl), NH SO₂N(C₁₋₈ alkyl)₂, NH SO₂NH₂.

In any aspect or embodiment described herein, q is an integer greater than or equal to 1.

In any aspect or embodiment described herein, e.g., where q of the linker is greater than 2, (A^{L})_{q} is a group which is A^{L}₁ and (A^{L})_{q} wherein the linker couples a PTM to a ULM.

In any aspect or embodiment described herein, e.g., where q of the linker is 2, A^{L}₂ is a group which is connected to A^{L}₁ and to a ULM.

In any aspect or embodiment described herein, e.g., where q of the linker is 1, the structure of the linker group L is -A^{L}₁-, and A^{L}₁ is a group which connects a ULM moiety to a PTM moiety.

In any aspect or embodiment described herein, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(heterocycloalkyl)-, —NR(CH₂CH₂O)ₙ-(lower alkyl)-O—CH₂—, —NR(CH₂CH₂O)ₙ-(heterocycloalkyl)-O—CH₂—, —NR(CH₂CH₂O)ₙ-Aryl-O—CH₂—, —NR(CH₂CH₂O)ₙ-(heteroaryl)-O—CH₂—, —NR(CH₂CH₂O)ₙ-(cyclo alkyl)-O-(heteroaryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)1-, —NR(CH₂CH₂)ₙ-(cycloalkyl)-O-(heterocyclyl)-CH₂, —NR(CH₂CH₂)ₙ-(heterocyclyl)-(heterocyclyl)-CH₂, and —N(R₁R₂)-(heterocyclyl)-CH₂; where
n of the linker can be 0 to 10;
R of the linker can be H, or lower alkyl; and
R₁ and R₂ of the linker can form a ring with the connecting N.

In any aspect or embodiment described herein, the linker (L) includes an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl, and including all implied subranges, e.g., C1-C10, C1-C20; C2-C10, C2-20; C10-C20, C10-C50 etc.), wherein each carbon is optionally independently substituted or replaced with (1) a heteroatom selected from N, O, S, P, or Si atoms that has an appropriate number of hydrogens, substitutions, or both to complete valency, (2) an optionally substituted cycloalkyl or bicyclic cycloalkyl, (3) an optionally substituted heterocycloalkyl or bicyclic heterocycloalkyl, (4) an optionally substituted aryl or bicyclic aryl, or (5) optionally substituted heteroaryl or bicyclic heteroaryl. In any aspect or embodiment described herein, the linker (L) does not have heteroatom-heteroatom bonding (e.g., no heteroatoms are covalently linked or adjacently located).

In any aspect or embodiment described herein, the linker (L) includes an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl), wherein:
each carbon is optionally independently substituted or replaced with CR^{L1}R^{L2}, S, SO, SO₂, NR^{L3}, SO₂NR^{L3}, SONR^{L3}, CONR^{L3}, NR^{L3}CONR¹⁴, NR^{L3}SO₂NR^{L4}, CO, CR^{L1}═CR^{L2}, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 1-6 R^{L1} and/or R^{L2} groups, C₅₋₁₃ spirocycloalkyl optionally substituted with 1-9 R^{L1} and/or R^{L2} groups, C₃₋₁₁ heterocyclyl optionally substituted with 1-6R^{L1} and/or R^{L2} groups, C₅₋₁₃ spiroheterocyclyl optionally substituted with 1-8 R^{L1} and/or R^{L2} groups, aryl optionally substituted with 1-6 R^{L1} and/or R^{L2} groups, heteroaryl optionally substituted with 0-6 R^{L1} and/or R^{L2} groups, where R^{L1} or R^{L2}, each independently are optionally linked to other groups to form a cycloalkyl and/or a heterocyclyl moiety, optionally substituted with 1-4 R^{L5} groups; and
R^{L1}, R^{L2}, R^{L3}, R^{L4} and R^{L5} are, each independently, H, halo, C₁₋₈ alkyl, OC₁₋₈ alkyl, SC₁₋₈ alkyl, NHC₁₋₈ alkyl, N(C₁₋₈alkyl)₂, C₃₋₁₁cycloalkyl, aryl, heteroaryl, C₃₋₁₁heterocyclyl, OC₃₋₈cycloalkyl, SC₃₋₈ cycloalkyl, NHC₃₋₈ cycloalkyl, N(C₃₋₈ cycloalkyl)₂, N(C₃₋₈ cycloalkyl)(C₁₋₈alkyl), OH, NH₂, SH, SO₂C₁₋₈ alkyl, P(O)(OC₁₋₈ alkyl)(C₁₋₈ alkyl), P(O)(OC₁₋₈ alkyl)₂, CC—C₁₋₈ alkyl, CCH, CH═CH(C₁₋₈ alkyl), C(C₁₋₈ alkyl)═CH(C₁₋₈ alkyl), C(C₁₋₈ alkyl)═C(C₁₋₈alkyl)₂, Si(OH)₃, Si(C₁₋₈ alkyl)₃, Si(OH)(C₁₋₈ alkyl)₂, COC₁₋₈ alkyl, CO₂H, halogen, CN, CF₃, CHF₂, CH₂F, NO₂, SF₅, SO₂NHC₁₋₈ alkyl, SO₂N(C₁₋₈ alkyl)₂, SONHC₁₋₈ alkyl, SON(C₁₋₈ alkyl)₂, CONHC₁₋₈ alkyl, CON(C₁₋₈ alkyl)₂, N(C₁₋₈ alkyl)CONH(C₁₋₈ alkyl), N(C1-8alkyl)CON(C₁₋₈ alkyl)₂, NHCONH(C₁₋₈ alkyl), NHCON(C₁₋₈ alkyl)₂, NHCONH₂, N(C₁₋₈alkyl)SO₂NH(C₁₋₈ alkyl), N(C₁₋₈ alkyl) SO₂N(C₁₋₈ alkyl)₂, NH SO₂NH(C₁₋₈ alkyl), NH SO₂N(C₁₋₈ alkyl)₂, NH SO₂NH₂.

In any aspect or embodiment described herein, the linker group is an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl, and including all implied subranges, e.g., C1-C10, C1-C20; C2-C10, C2-20; C10-C20, C10-C50 etc.), wherein each carbon atom optionally substituted or replaced with: a O, N, S, P or Si atom that has an appropriate number of hydrogens, substitutions (e.g., OH, halo, alkyl, methyl, ethyl, haloalkyl, hydroxyalkyl, alkoxy, methoxy, etc.), or both to complete valency; an optionally substituted aryl (e.g., an optionally substituted C5 or C6 aryl) or bicyclic aryl (e.g., an optionally substituted C5-C20 bicyclic heteroaryl); an optionally substituted heteroaryl (e.g., an optionally substituted C5 or C6 heteroaryl) or bicyclic heteroaryl (e.g., an optionally substituted heteroaryl or bicyclic heteroaryl having one or more heteroatoms selected from N, O, S, P, and Si that has an appropriate number of hydrogens, substitutions (e.g., OH, halo, alkyl, methyl, ethyl, haloalkyl, hydroxyalkyl, alkoxy, methoxy, etc.), or both to complete valency); an optionally substituted C1-C6 alkyl; an optionally substituted C1-C6 alkenyl; an optionally substituted C1-C6 alkynyl; an optionally substituted cycloalkyl (e.g., an optionally substituted C3-C7 cycloalkyl) or bicyclic cycloalkyl (e.g., an optionally substituted C5-C20 bicyclic cycloalkyl); or an optionally substituted heterocycloalkyl (e.g., an optionally substituted 3-, 4-, 5-, 6-, or 7-membered heterocyclic group) or bicyclicheteroalkyl (e.g., an optionally substituted heterocycloalkyl bicyclicheteroalkyl having one or more heteroatoms selected from N, O, S, P, or Si atoms that has an appropriate number of hydrogens, substitutions (e.g., OH, halo, alkyl, methyl, ethyl, haloalkyl, hydroxyalkyl, alkoxy, methoxy, etc.), or both to complete valency). In any aspect or embodiment described herein, the optionally substituted alkyl linker is optionally substituted with one or more OH, halo, linear or branched C1-C6 alkyl (such as methyl or ethyl), linear or branched C1-C6 haloalkyl, linear or branched C1-C6 hydroxyalkyl, or linear or branched C1-C6 alkoxy (e.g., methoxy).

In any aspect or embodiment described herein, the linker (L) does not have heteroatom-heteroatom bonding (e.g., no heteroatoms are covalently linked or adjacently located).

In any aspect or embodiment described herein, the linker (L) includes about 1 to about 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) alkylene glycol units that are optionally substituted, wherein carbon or oxygen may be substituted or replaced with a heteroatom selected from N, S, P, or Si atoms with an appropriate number of hydrogens to complete valency.

In any aspect or embodiment described herein, the linker (L) is represented by the chemical structure:

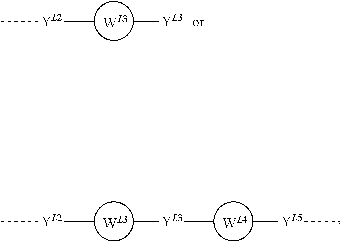

wherein:
the [symbol] of the chemical linking moiety is the site of attachment to the VLM or the PTM;
Y^{L2} is a bond, or a unsubstituted or substituted linear or branched C1-C4 alkyl (e.g., optionally substituted with a halogen, C1-3 alkyl, methyl, or ethyl);
W^{L3} is a 3-7 membered ring (e.g., 4-6 membered cycloalkyl or heterocycloalkyl) or a 8-12 member spirocyclic, each with 0-4 heteroatoms (e.g., 0-4 heteroatoms independently selected from N, O, and S) and optionally substituted with halogen or methyl;
Y^{L3} is a bond or a C1-C35 alkyl (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, or C₃₅ alkyl), wherein one or more C atoms are optionally replaced with O,

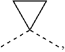

or NH, and each carbon is optionally substituted with a halogen, ═O, a methyl or ethyl, and each nitrogen is optionally substituted with a halogen, a methyl, or ethyl;
Y_{L4} is bond, O, or an unsubstituted or substituted linear or branched C1-C6 alkyl, wherein one or more carbons are optionally replaced O, NH, or NCH₃, and optionally substituted with a halogen or methyl;
W^{L4} is a 3-8 membered ring (e.g., 4-6 membered cycloalkyl or heterocycloalkyl, or

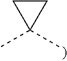

or a 5-8 member spirocyclic, each with 0-4 heteroatoms (e.g., 0-4 heteroatoms independently selected from N, O, and S) and optionally substituted with halogen (e.g., F, Cl, Br), or methyl; and
Y^{L5} is a bond or an unsubstituted or substituted C1-C6 alkyl, where one or more C atoms are optionally replaced with O and optionally substituted with a halo (e.g., F, Cl, Br), or methyl

In any aspect or embodiment described herein, the unit A^{L} of the linker (L) comprises a structure selected from the group consisting of:

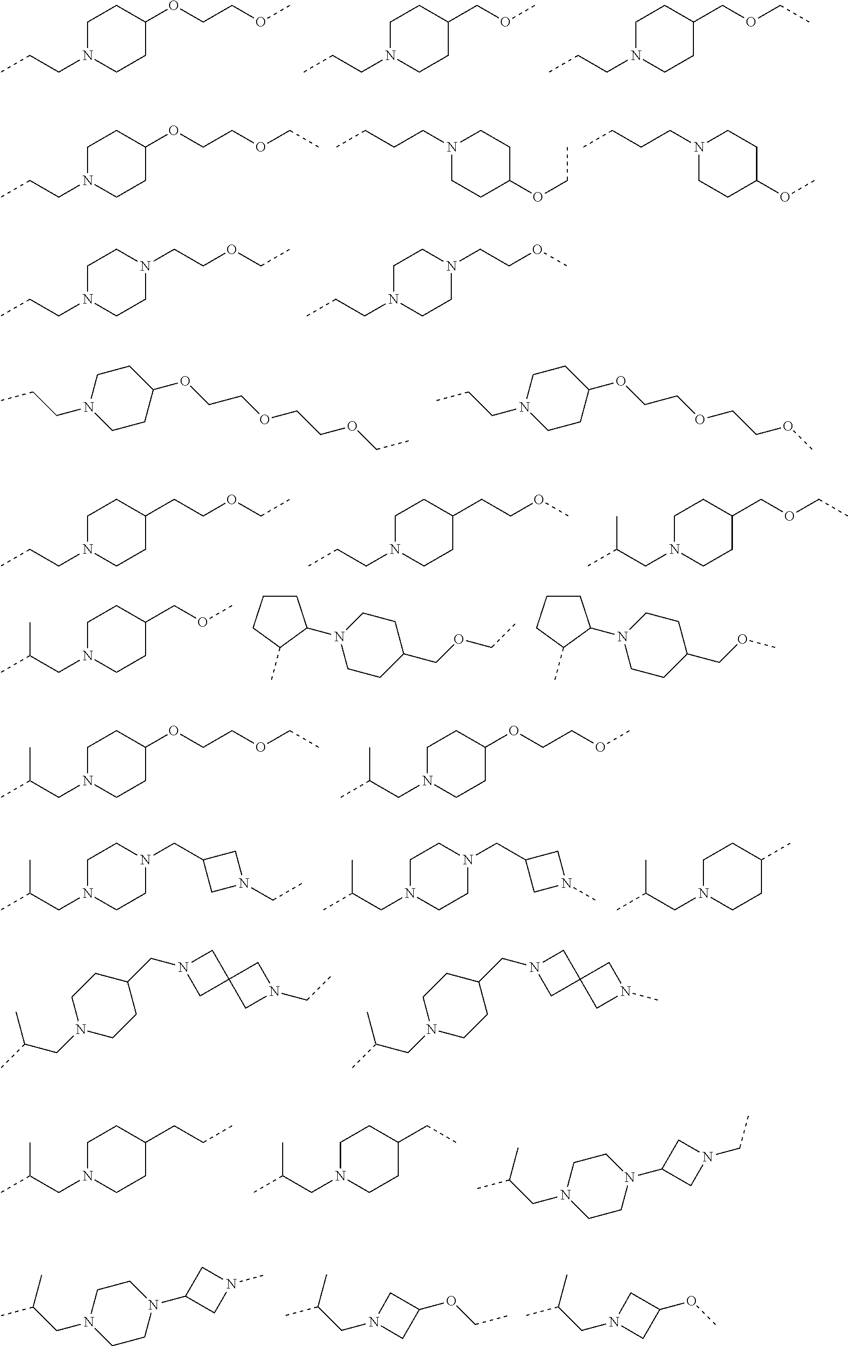

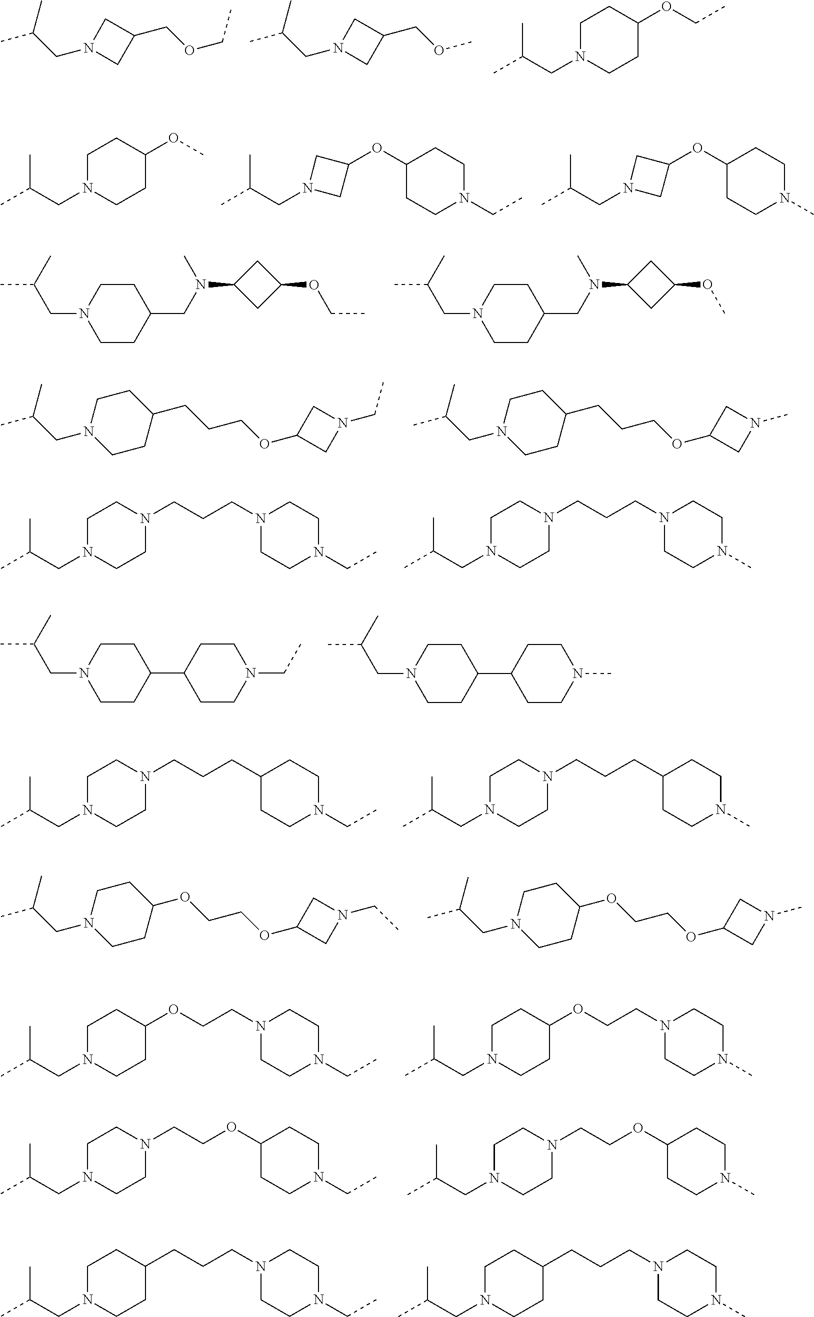

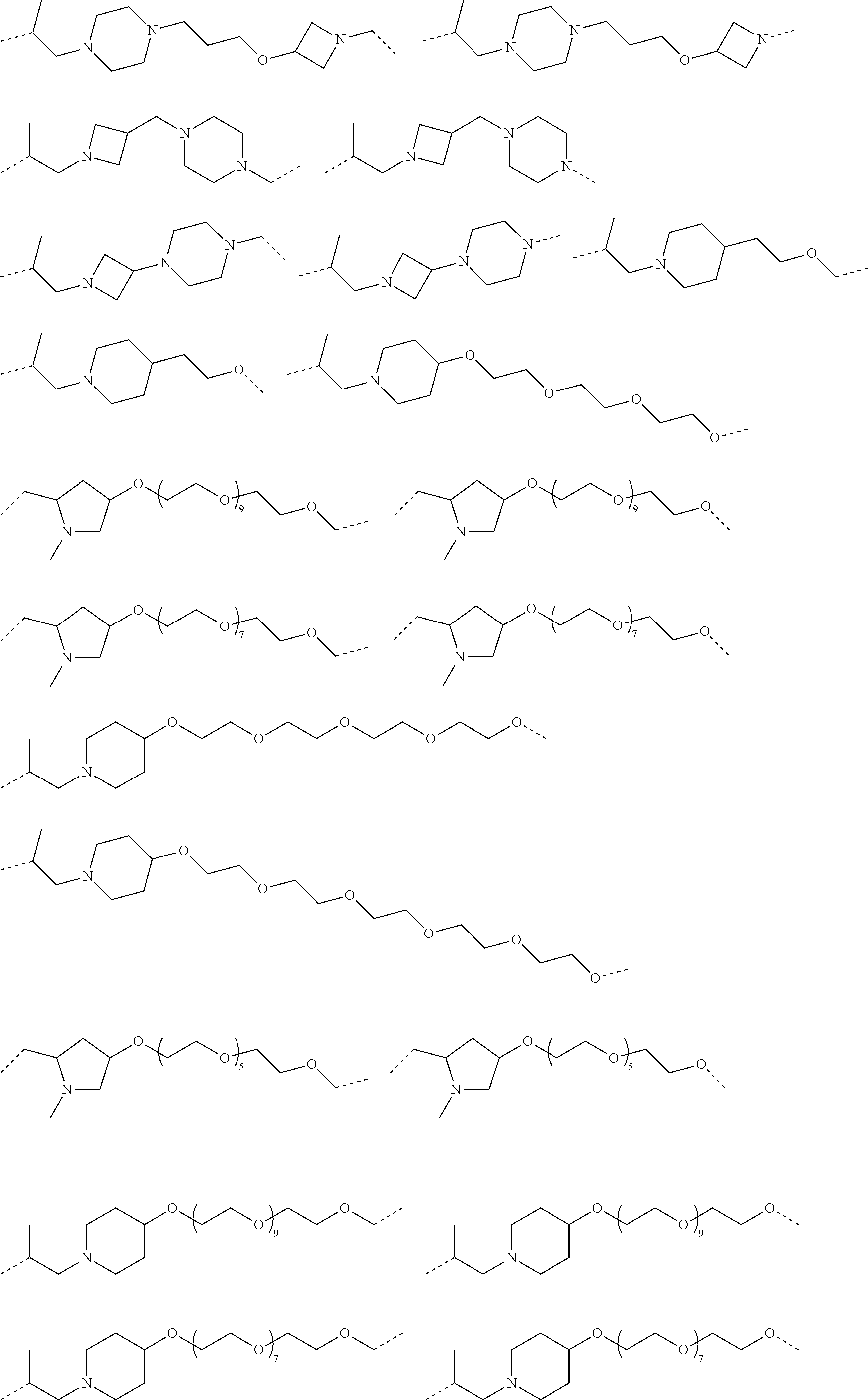

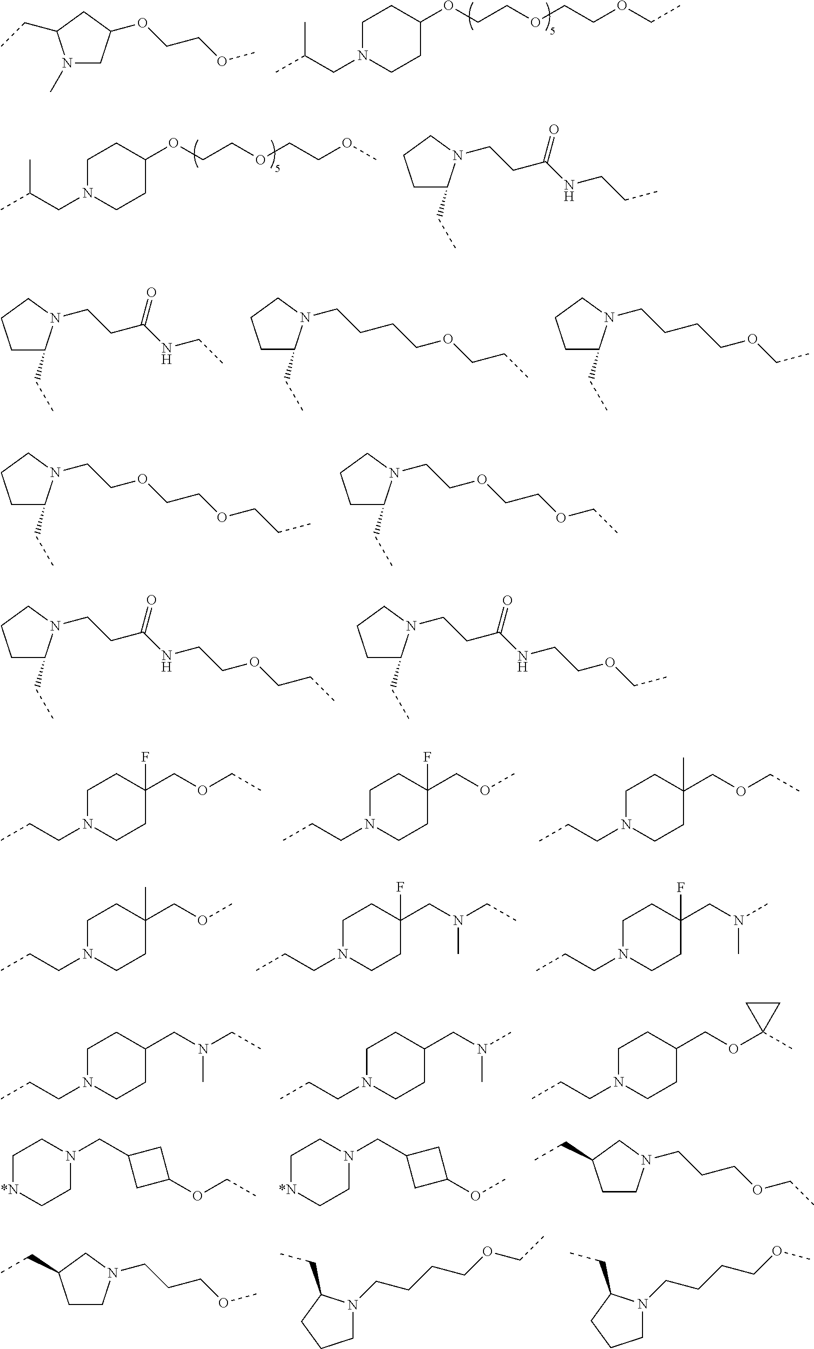

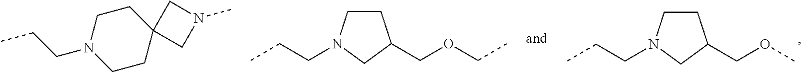

wherein the [symbol] indicates the point of attachment with the PTM or the VLM.

In any aspect or embodiment described herein, the unit A^{L} of the linker (L) comprises a structure selected from the group consisting of:

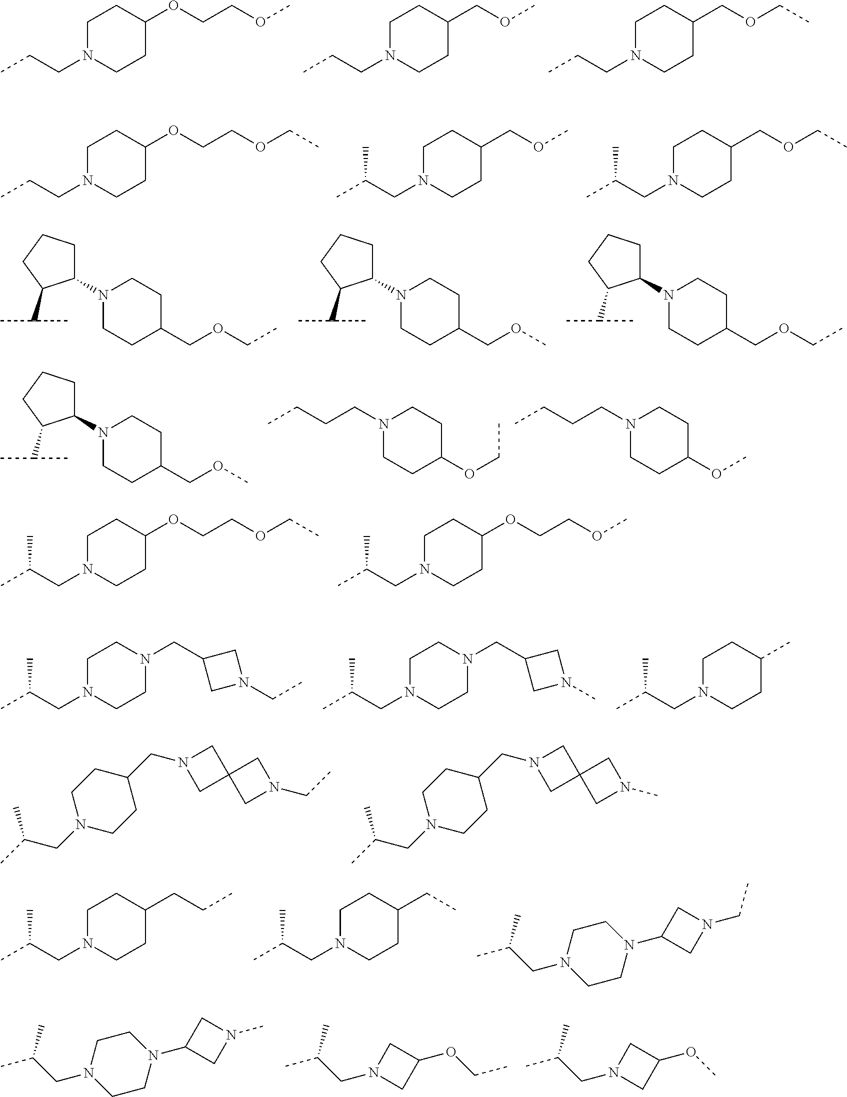

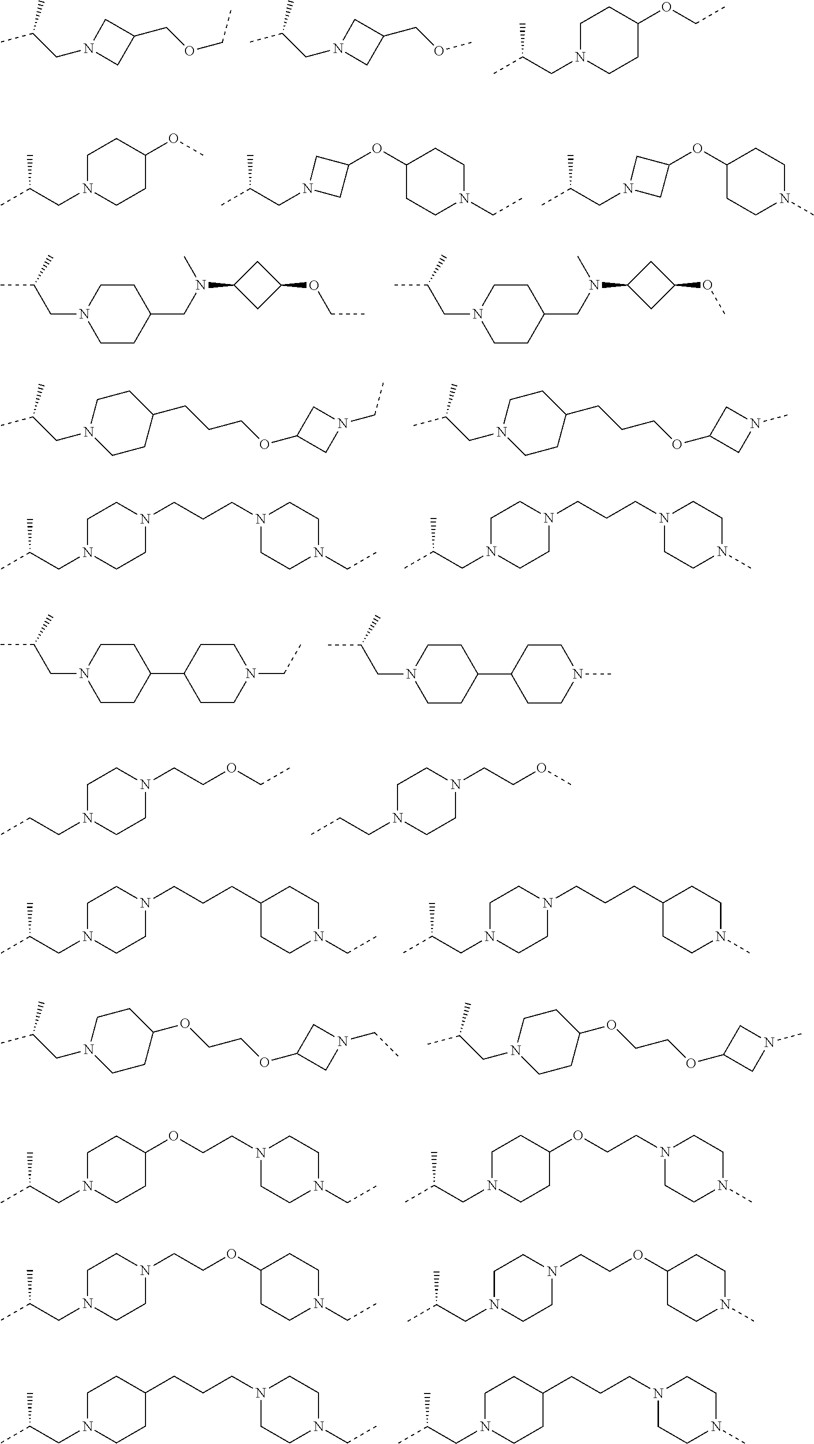

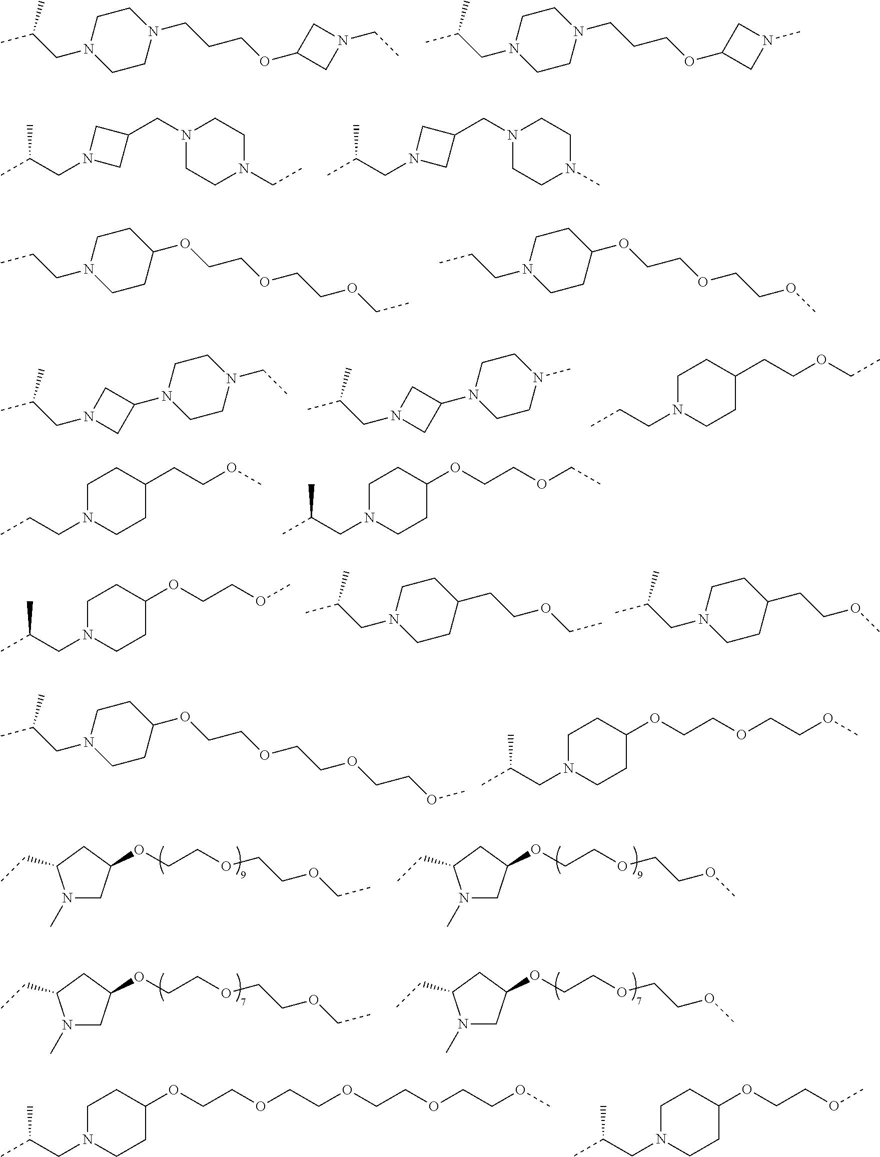

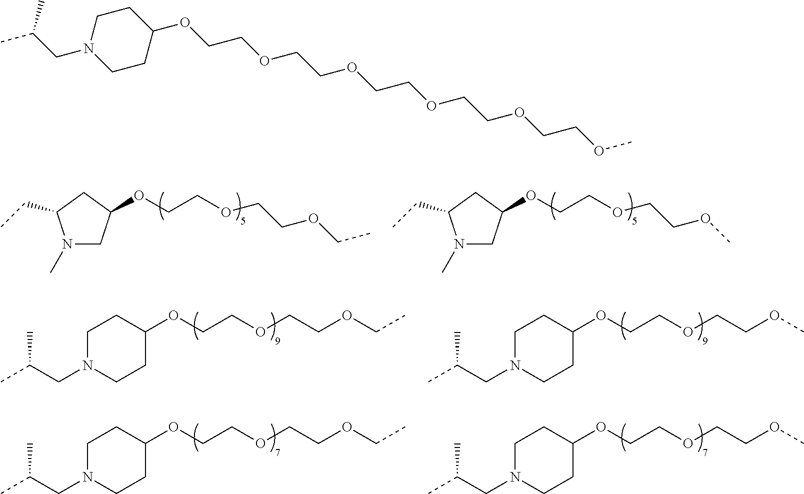

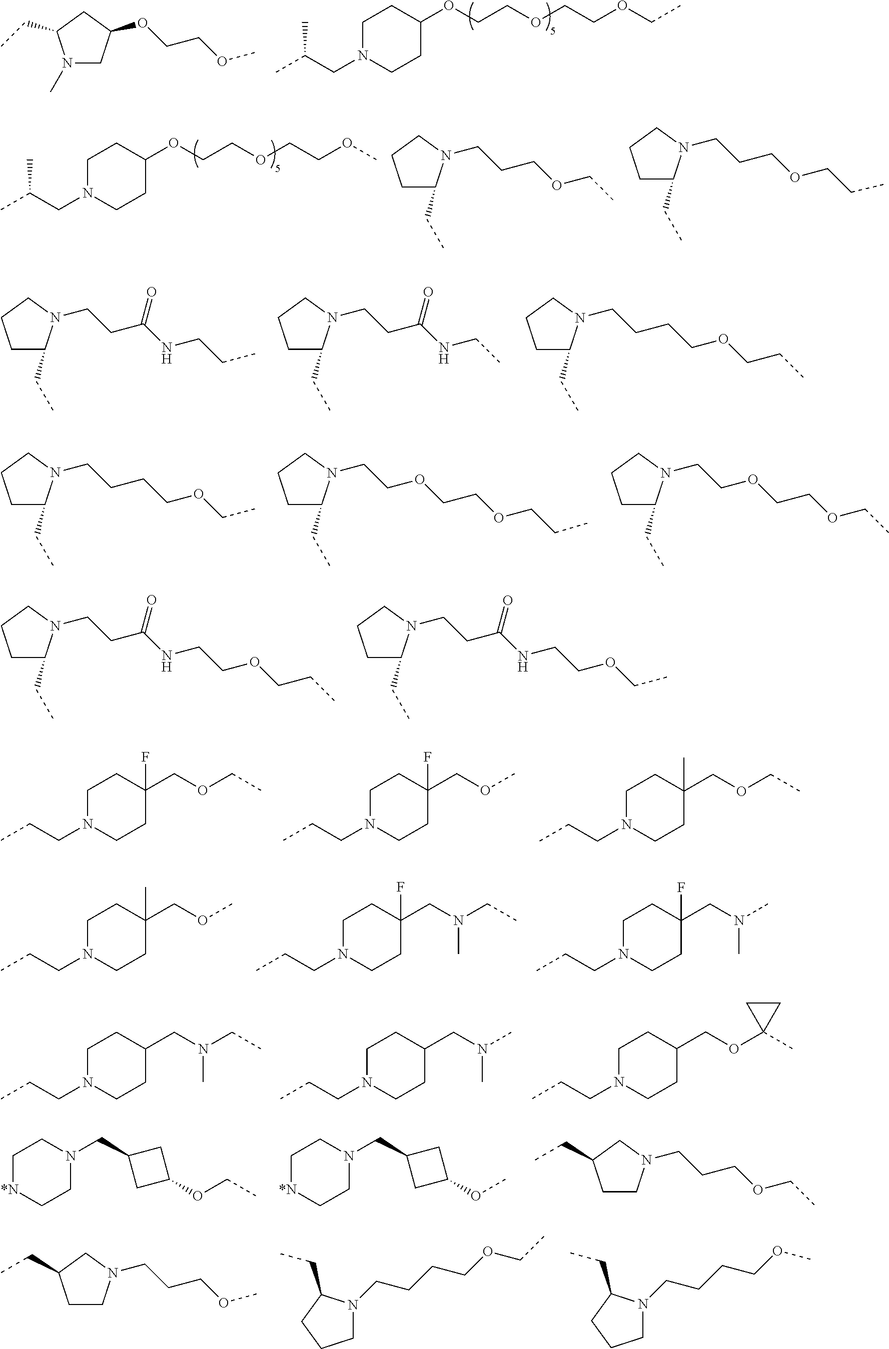

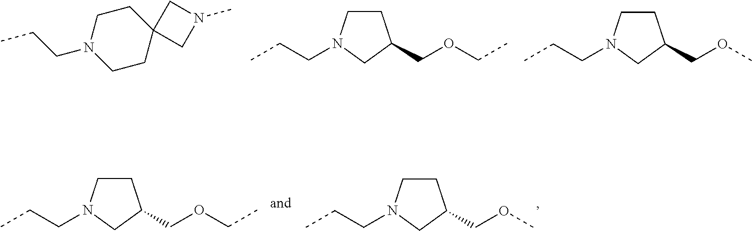

wherein the [symbol] indicates the point of attachment with the PTM or the VLM.

In any aspect or embodiment described herein, the linker (L) comprises a structure selected from the structure shown below:

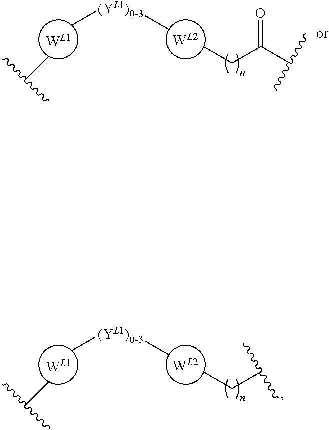

wherein:
W^{L1} and W^{L2} are each independently absent, a 4-8 membered ring with 0-4 heteroatoms, optionally substituted with R^{Q}, each R^{Q} is independently a H, halo, OH, CN, CF₃, optionally substituted linear or branched C₁-C₆ alkyl, optionally substituted linear or branched C₁-C₆ alkoxy, or 2 R^{Q} groups taken together with the atom they are attached to, form a 4-8 membered ring system containing 0-4 heteroatoms;
Y^{L1} is each independently a bond, optionally substituted linear or branched C₁-C₆ alkyl and optionally one or more C atoms are replaced with O or NR^{YL1}, optionally substituted C₁-C₆ alkene and optionally one or more C atoms are replaced with O, optionally substituted C₁-C₆ alkyne, and optionally one or more C atoms are replaced with O, or optionally substituted linear or branched C₁-C₆ alkoxy;
R^{YL1} is H, or optionally substituted linear or branched C₁₋₆ alkyl;
n is 0-10; and
[symbol] and [symbol] indicates the attachment point to the PTM or ULM moieties.

In any aspect or embodiment described herein, the linker (L) comprises a structure selected from the structure shown below:

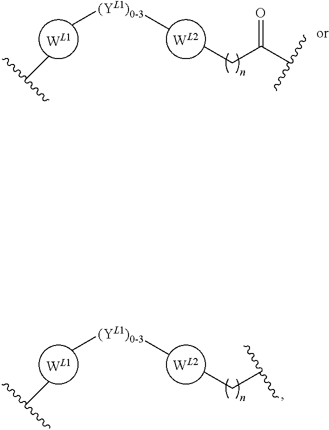

wherein:
W^{L1} and W^{L2} are each independently absent, piperazine, piperidine, morpholine, optionally substituted with R^{Q}, each R^{Q} is independently a H, —Cl—, —F—, OH, CN, CF₃, optionally substituted linear or branched C₁-C₆ alkyl (e.g. methyl, ethyl), optionally substituted linear or branched C₁-C₆ alkoxy (e.g. methoxy, ethoxy);
Y^{L1} is each independently a bond, optionally substituted linear or branched C₁-C₆ alkyl and optionally one or more C atoms are replaced with O or NR^{Y}″; optionally substituted C₁-C₆ alkene and optionally one or more C atoms are replaced with O, optionally substituted C₁-C₆ alkyne and optionally one or more C atoms are replaced with O, or optionally substituted linear or branched C₁-C₆ alkoxy;
R^{YL1} is H, or optionally substituted linear or branched C₁₋₆ alkyl (e.g. methyl, ethyl);
n is 0-10; and
[symbol] and [symbol] indicates the attachment point to the PTM or ULM moieties.

In any aspect or embodiment described herein, the linker (L) comprises a structure selected from the structure shown below:

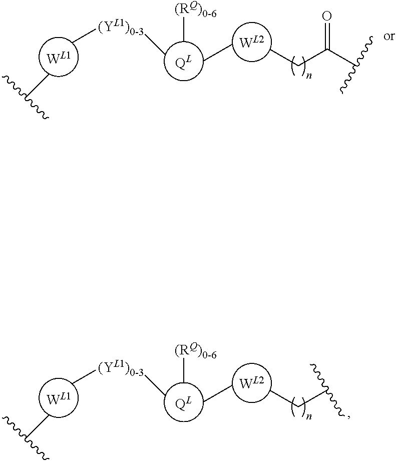

wherein:
W^{L1} and W^{L2} are each independently absent, aryl, heteroaryl, cyclic, heterocyclic, C₁₋₆ alkyl and optionally one or more C atoms are replaced with O or NR^{YL1}, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q}, each R^{Q} is independently a H, halo, OH, CN, CF₃, hydroxyl, nitro, CCH, C₂₋₆ alkenyl, C₂₋₆ alkynyl, optionally substituted linear or branched C₁-C₆ alkyl, optionally substituted linear or branched C₁-C₆ alkoxy, optionally substituted OC₁₋₃ alkyl (e.g., optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, optionally substituted linear or branched C₁-C₆ alkyl and optionally one or more C atoms are replaced with O; optionally substituted linear or branched C₁-C₆ alkoxy;
Q^{L} is a 3-6 membered alicyclic, bicyclic or aromatic ring with 0-4 heteroatoms, optionally bridged, optionally substituted with 0-6 R^{Q}, each R^{Q} is independently H, optionally substitute linear or branched C₁₋₆ alkyl (e.g., optionally substituted by 1 or more halo, C₁₋₆ alkoxyl), or 2 R^{Q} groups taken together with the atom they are attached to, form a 3-8 membered ring system containing 0-2 heteroatoms;
R^{YL1}, R^{YL2} are each independently H, OH, optionally substituted linear or branched C₁₋₆ alkyl (e.g., optionally substituted by 1 or more halo, C₁₋₆ alkoxyl), or R¹, R² together with the atom they are attached to, form a 3-8 membered ring system containing 0-2 heteroatoms;
n is 0-10; and
[symbol] and [symbol] indicates the attachment point to the PTM or ULM moieties.

In any aspect or embodiment described herein, the linker (L) comprises a structure selected from the structure shown below:

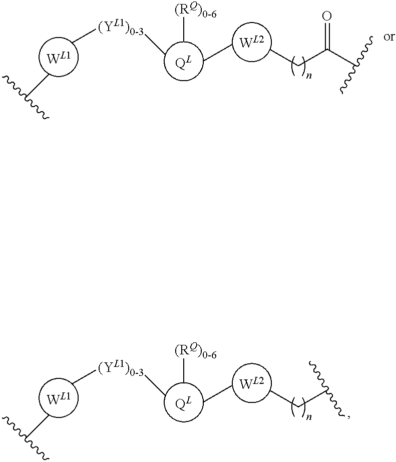

wherein:
W^{L1} and W^{L2} are each independently absent, cyclohexane, cyclopentane, piperazine, piperidine, morpholine, C₁₋₆ alkyl and optionally one or more C atoms are replaced with O or NR^{YL1}, C₁₋₆ alkene and optionally one or more C atoms are replaced with O, C₁₋₆ alkene and optionally one or more C atoms are replaced with O, or C₁₋₆ alkyne and optionally one or more C atoms are replaced with O, each optionally substituted with R^{Q}, each R^{Q} is independently a H, —Cl, —F, OH, CN, CF₃, hydroxyl, optionally substituted linear or branched C₁-C₆ alkyl (e.g., methyl, ethyl), or optionally substituted linear or branched C₁-C₆ alkoxy;
Y^{L1} is each independently a bond, NR^{YL1}, O, CR^{YL1}R^{YL2}, C═O, optionally substituted linear or branched C₁-C₆ alkyl and optionally one or more C atoms are replaced with O or NR^{YL1}, C₁₋₆ alkene and optionally one or more C atoms are replaced with O, C₁₋₆ alkyne and optionally one or more C atoms are replaced with O, or optionally substituted linear or branched C₁-C₆ alkoxy;
Q^{L} is a 3-6 membered heterocyclic, heterobicyclic, or heteroaryl ring, optionally substituted with 0-6 R^{Q}, each R^{Q} is independently H, or optionally substituted linear or branched C₁₋₆ alkyl (e.g., optionally substituted by 1 or more halo, C₁₋₆ alkoxyl);
R^{YL1}, R^{YL2} are each independently H, optionally substituted linear or branched C₁₋₆ alkyl (e.g., methyl, ethyl, optionally substituted by 1 or more halo, C₁₋₆ alkoxyl);
n is 0-10; and
[symbol] and [symbol] indicates the attachment point to the PTM or ULM moieties.

### Exemplary PTMs

In one aspect of the disclosure, the PTM group (also referred as the KTM group) binds to the target protein, KRas or mutated form thereof, such as KRas^{G12C}.

The compositions described below exemplify members of KRas binding moieties (e.g., KRas^{G12C} binding moiety) that can be used according to the present invention. These binding moieties are linked to the ubiquitin ligase binding moiety (VLM) preferably through a chemical linking group in order to present the KRas protein, such as KRas^{G12C}, in proximity to the ubiquitin ligase for ubiquitination and subsequent degradation.

In certain contexts, the term “target protein” is used to refer to the KRas protein, a member of the RAS/MAPK pathway, which is a target protein to be ubiquitinated and degraded. In other contexts, the term “target protein” is used to refer to a mutated form of the KRas protein, such as a gain-of-function KRas mutant protein or a KRas protein having one or mutation selected from the group consisting of codon 12 missense mutation, codon 12 missense mutation, exon 2 mutation, G12V, G12C, G12D, G12A, G13D, exon 3 mutation, codon 61 missense mutation, exon 4 mutation, G12R, Q61H, G12S, A146T, G13C, Q61R, Q61L, A146V, codon 117 missense mutation, K117N, Q61K, G12F, codon 59 missense mutation, A59T, or combinations thereof.

In any of the aspects or embodiments described herein, the PTM is a small molecule that selectively or preferentially binds to a KRas protein having at least one mutation that is a G12C mutation (e.g., KRas^{G12C}) compared to the PTM binding to a wildtype KRas. In any of the aspects or embodiments described herein, the PTM is a small molecule capable of selectively binding the KRas protein having at least one mutation that is a G12C mutation (e.g., KRas^{G12C}), wherein selectivity towards the KRas protein having at least one mutation that is a G12C mutation is at least 1-60 times (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 times) compared to the wild-type KRas. In any of the aspects or embodiments described herein, the PTM is a small molecule that binds the KRas protein having at least one mutation that is a G12C mutation (e.g., KRas^{G12C}), wherein selectivity towards the KRas protein having at least one mutation that is a G12C mutation is at least 1-1000 times (e.g., 50, 100, 200, 300, 400, 500, 600, 700, 800, 900 times) compared to the wild-type KRas.

The term “protein target moiety” or PTM is used to describe a small molecule which binds to KRas or mutated form thereof, such as KRas^{G12C}, and can be used to target the protein for ubiquitination and degradation.

The compositions described herein exemplify the use of some of these PTMs.

In any aspect or embodiment described herein, the PTM is represented by the chemical structure:

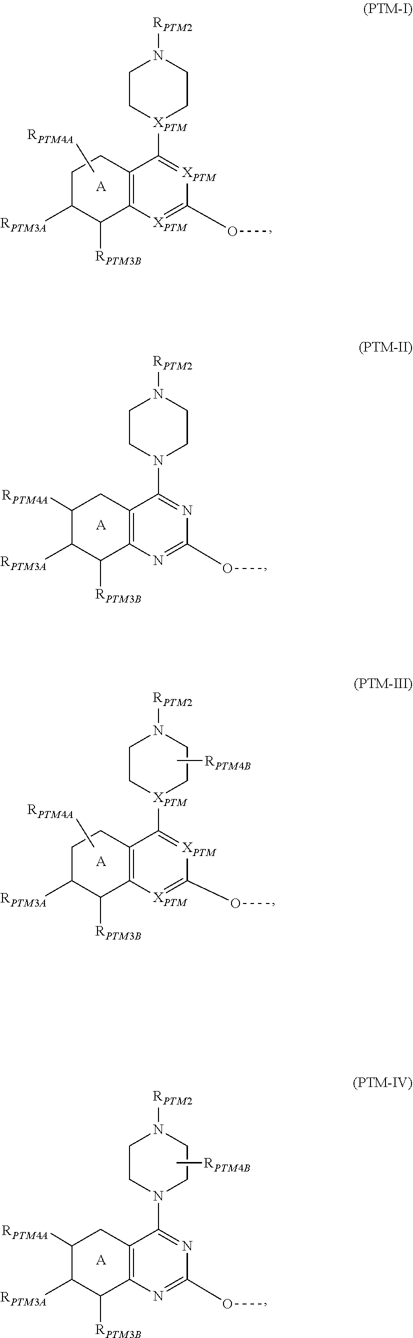

wherein:
the [symbol] of the PTM is the site of attachment to the VLM or the L coupling the VLM to the PTM;

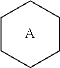

is an 6-membered aryl, 6-membered heteroaryl, or a 6-membered heterocycloalkyl, each optionally substituted with 1 or 2 halogens (e.g., Cl, F, or Br);
R_{PTM2} is —C(═O)C₂-C₄alkenyl, optionally substituted by a methyl, halogen (e.g., Cl, F, Br), amine (e.g., —NH₂, —NHCH₃, or —N(CH₃)₂), or a 3-6 membered heterocycloalkyl (e.g., a 6-membered heterocycloalkyl, a heterocycloalkyl having heteroatoms selected from O and N, or

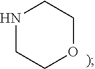

R_{PTM3A} is H, phenyl, pyridinyl, isoquinoline, or naphthalene

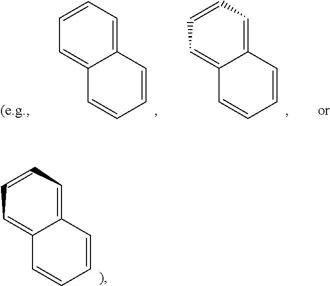

each optionally substituted by 1, 2, or 3 groups independently selected from OH, halogen (e.g., F, Cl, Br), amine (e.g., —NH₂, —NHCH₃, or —N(CH₃)₂), a linear or branched C1-C3 haloalkyl (e.g., a linear or branched C1-C3 fluoroalkyl or CF₃), —R_{PTM3C}, or a linear or branched C₁-C₃ alkyl (e.g., methyl or ethyl), wherein R_{PTM3C} is an indazole optionally substituted by 1, 2, or 3 groups independently selected from OH, halogen (e.g., F, Cl, Br), or a linear or branched C₁-C₃ alkyl (e.g., methyl or ethyl)

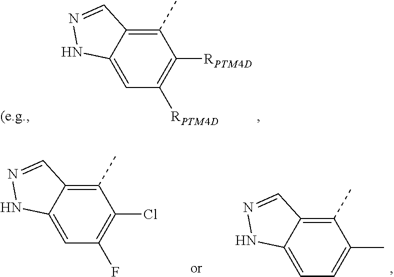

wherein each R_{PTM4D} is independently selected from a hydrogen, C₁-C₃ alkyl (e.g., methyl), or a halogen (e.g., F, Cl, Br)); R_{PTM3B} is H, halogen (e.g., Cl, F, Br), or —O—R_{PTM3C}, wherein R_{PTM3C} is optionally substituted by 1, 2, or 3 groups independently selected from OH, halogen (e.g., F, Cl, Br), or a linear or branched C₁-C₃ alkyl (e.g., methyl or ethyl)

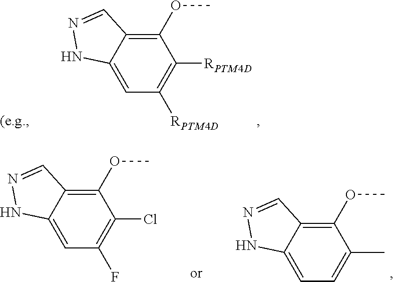

wherein each R_{PTM4D} is independently selected from a hydrogen, C₁-C₃ alkyl (e.g., methyl), or a halogen (e.g., F, Cl, Br));
R_{PTM4A} of PTM-I and PTM-III is absent (or H) or 1 or 2 independently selected halogen (e.g., Cl, F, Br);
R_{PTM4A} of PTM-II and PTM-IV is absent (or H) or a halogen (e.g., Cl, F, Br);
R_{PTM4B} is (1) H or absent, (2) —CH₂—CH₂—CN or —CH₂—CN, or (3) 1 or 2 independently selected C1-C3 alkyl (e.g., methyl or ethyl); and
each X_{PTM} is individually a CH or N.

In any aspect or embodiment described herein, the PTM is represented by the chemical structure:

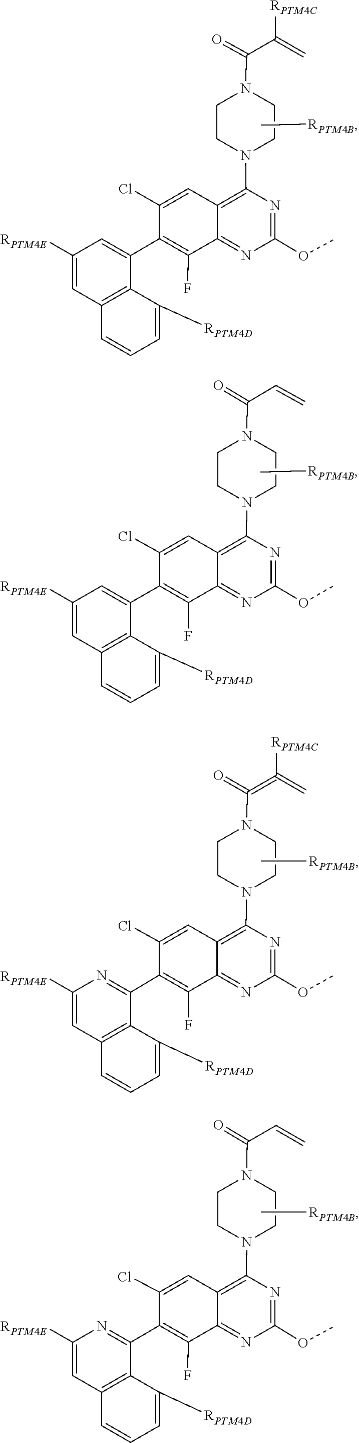

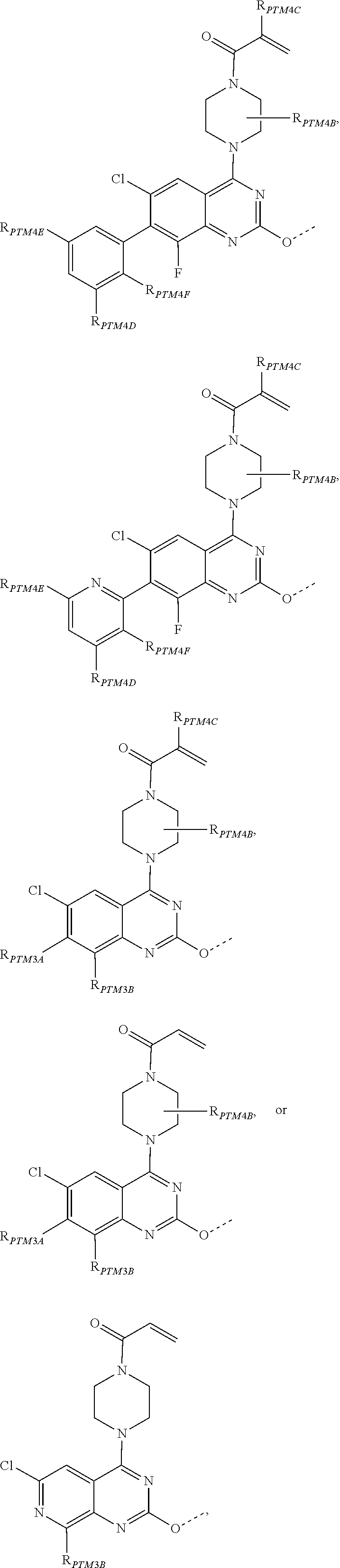

wherein:
the [symbol] of the PTM is the site of attachment to the VLM or the L coupling the VLM to the PTM;
R_{PTM3A} is indazole, optionally substituted by 1 or 2 groups independently selected from OH, methyl, and halogen (e.g., F, Cl, Br);
R_{PTM4B} is (1) absent (or H), (2) —CH₂—CH₂—CN or —CH₂—CN, or (3) 1 or 2 independently selected C₁-C₃ alkyl (e.g., methyl or ethyl);
R_{PTM4D} is a hydrogen, C₁-C₃ alkyl (e.g., methyl), or a halogen (e.g., F, Cl, Br);
R_{PTM4C} is H or halogen (e.g., Cl, F, Br);
R_{PTM4E} is H, OH, or amine (e.g., —NH₂, or —NHCH₃);
R_{PTM4F} is a hydrogen, C₁-C₃ alkyl (e.g., methyl), C₁-C₃ haloalkyl (e.g., C₁-C₃ fluoroalkyl or CF₃), or a halogen (e.g., F, Cl, Br); and
R_{PTM3B} is —O-indazole, optionally substituted by 1 or 2 groups independently selected from OH, methyl, and halogen (e.g., F, Cl, Br).

In any aspect or embodiment described herein, the PTM is represented by the chemical structure:

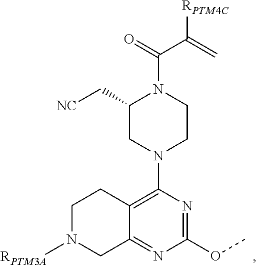

wherein:
the [symbol] of the PTM is the site of attachment to the VLM or the L coupling the VLM to the PTM;
R_{PTM3A} is:

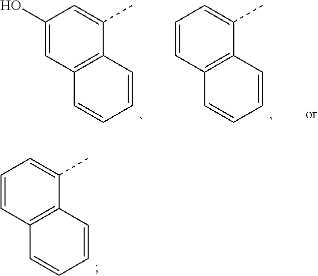

and
R_{PTM4C} is H or F.

In any aspect or embodiment described herein, the PTM is represented by the chemical structure:

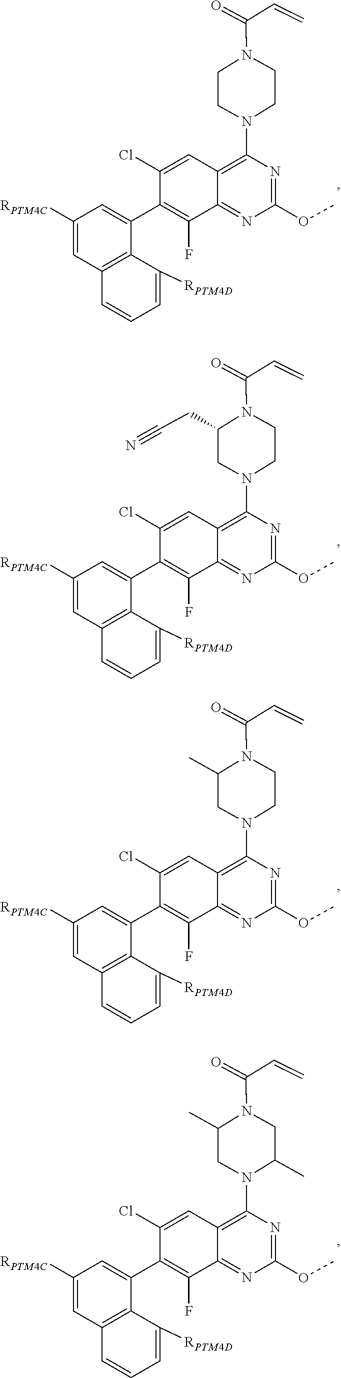

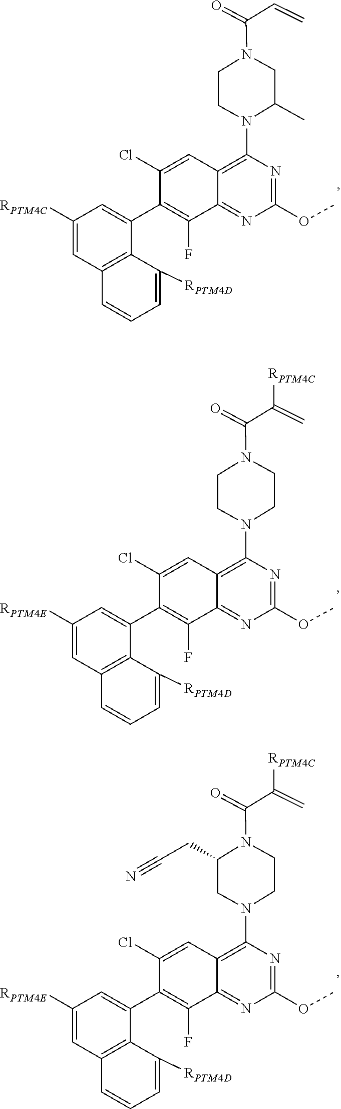

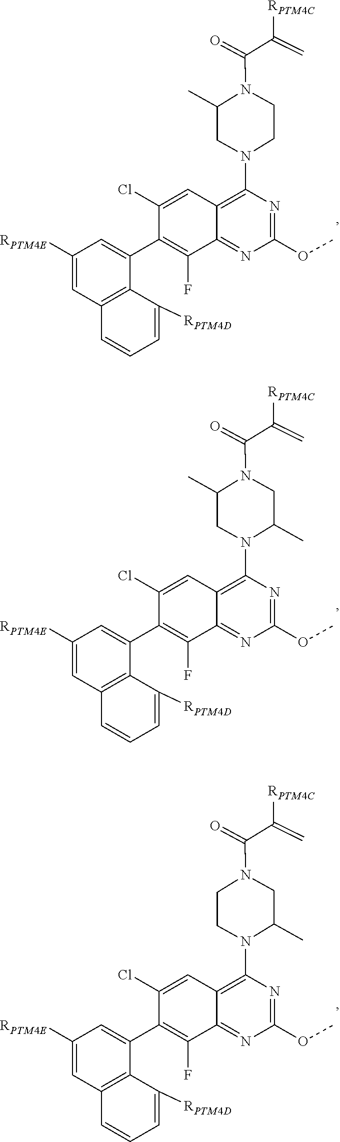

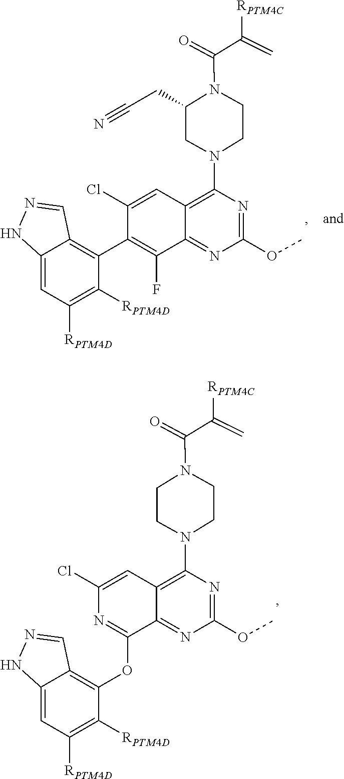

wherein: R_{PTM4C}, R_{PTM4D}, and R_{PTM4E} are each independently as defined in any other aspect or embodiment described herein; and the [symbol] of the PTM is the site of attachment to the VLM or the L coupling the VLM to the PTM.

In any aspect or embodiment described herein, the PTM is represented by the chemical structure:

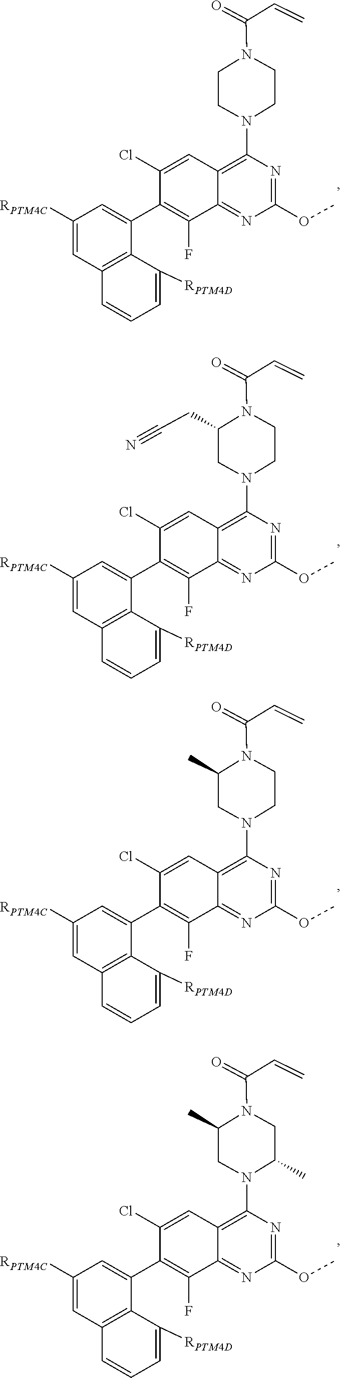

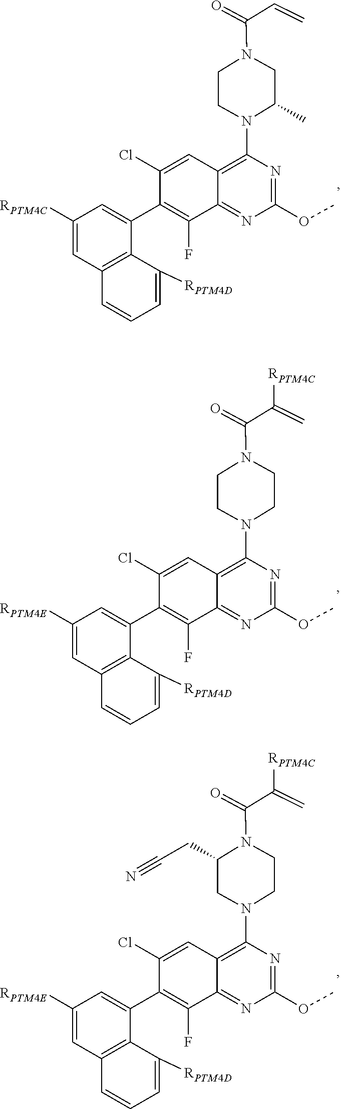

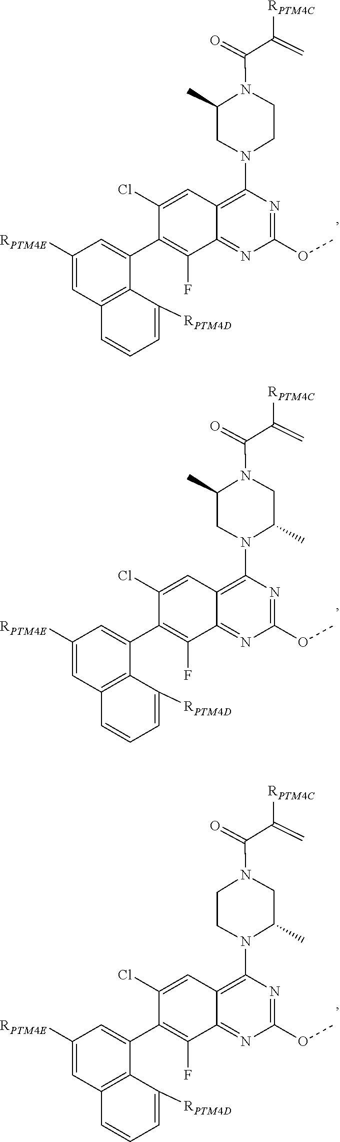

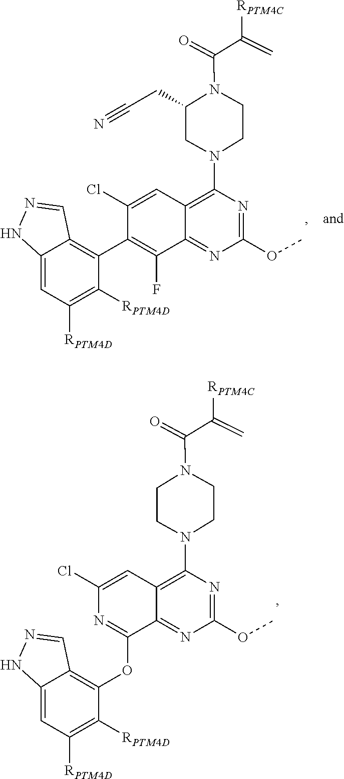

wherein: R_{PTM4C}, R_{PTM4D}, and R_{PTM4E} are each independently as defined in any other aspect or embodiment described herein; and the [symbol] of the PTM is the site of attachment to the VLM or the L coupling the VLM to the PTM.

In any aspect or embodiment described herein, the PTM is selected from the group consisting of:

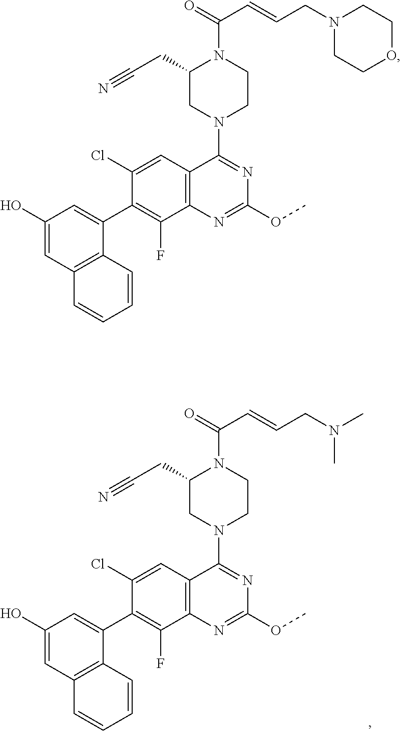

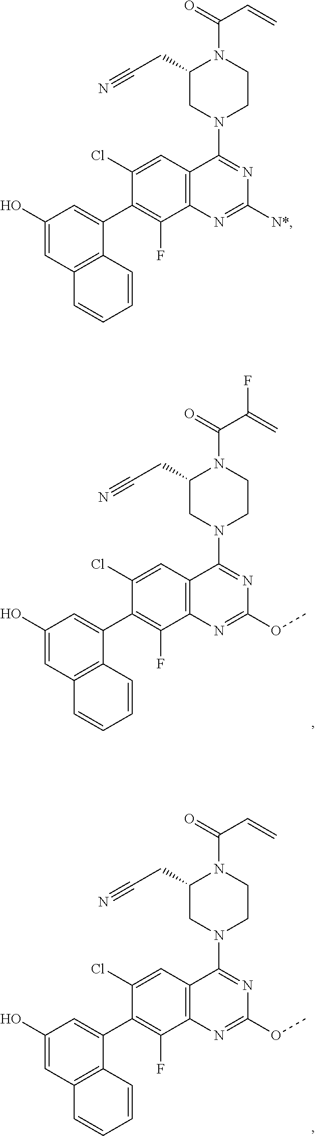

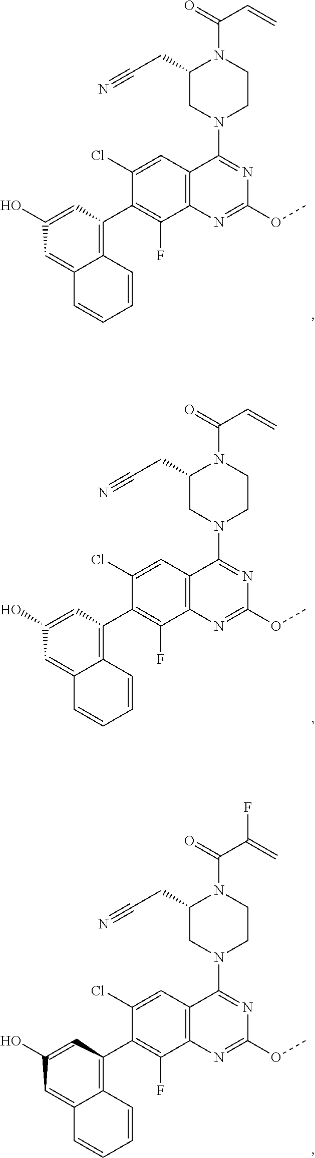

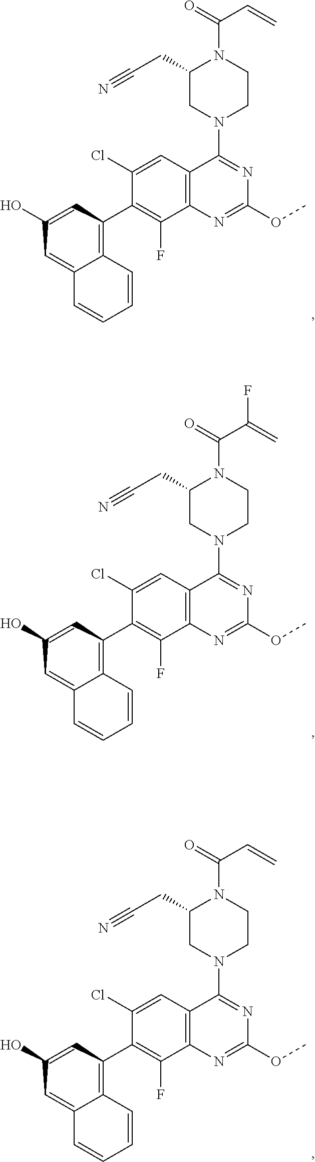

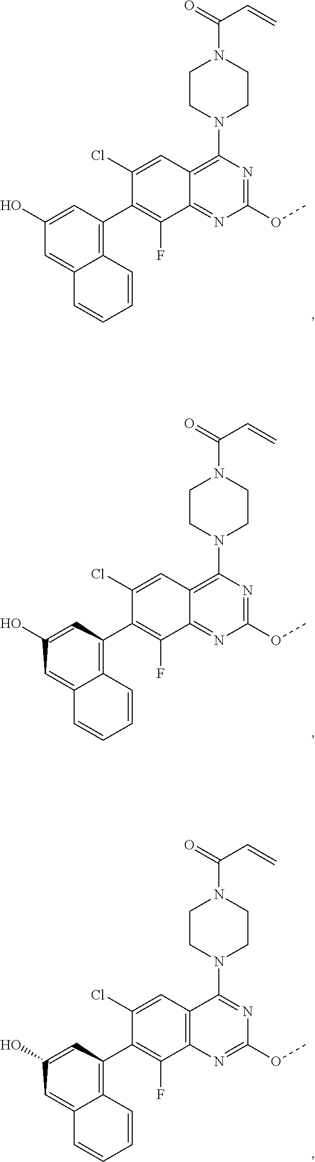

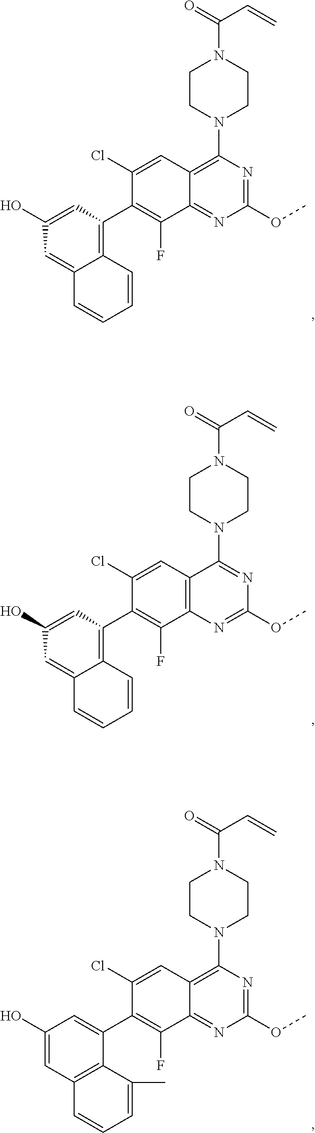

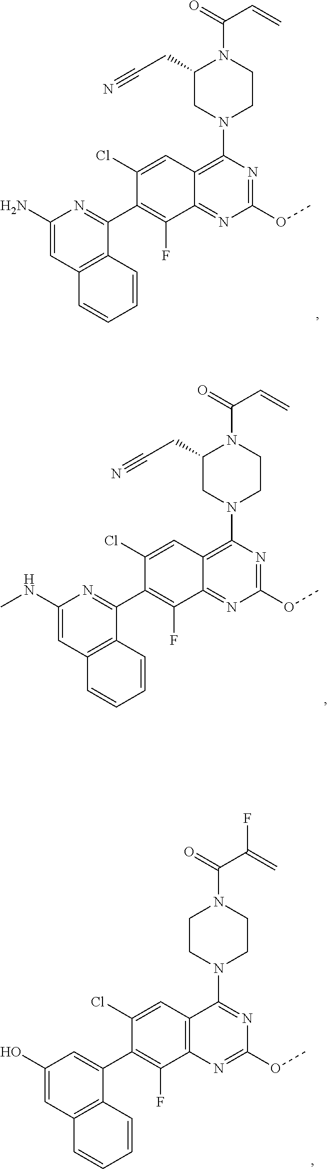

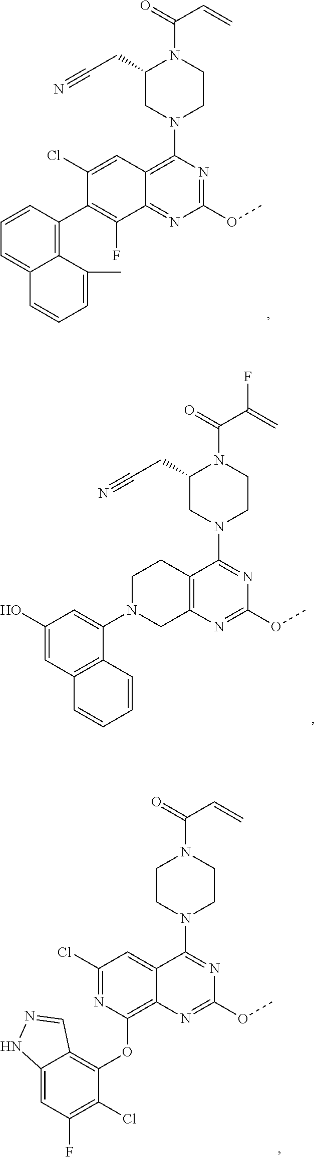

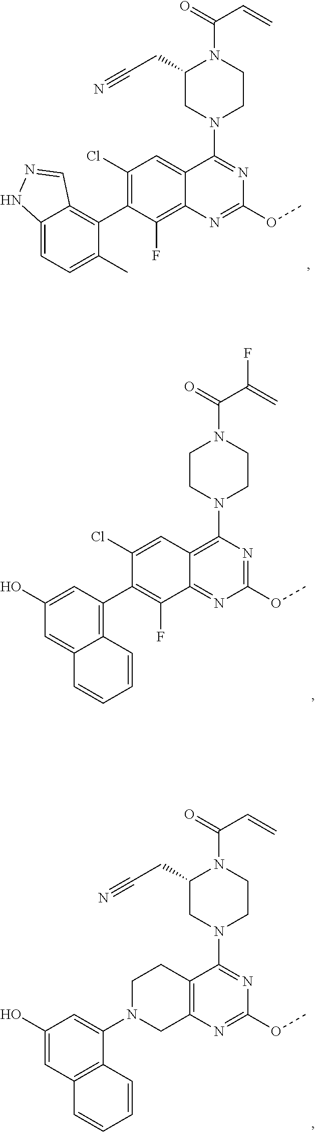

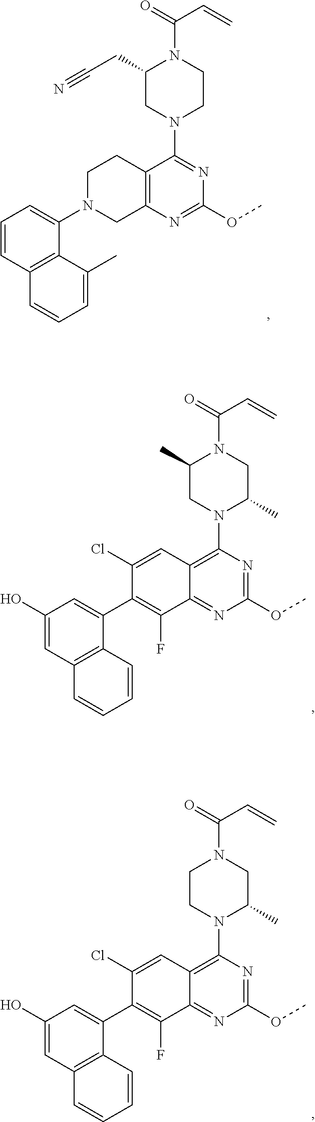

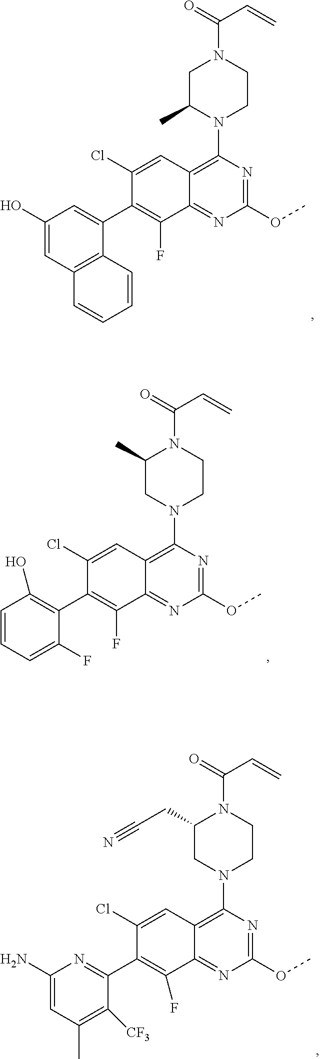

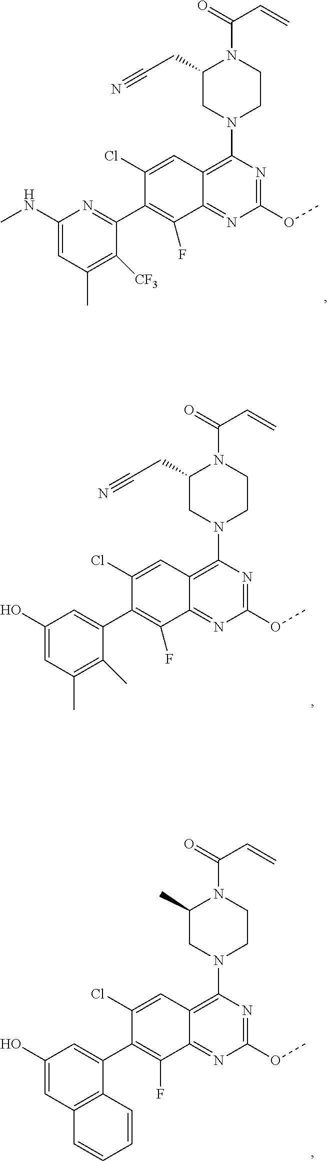

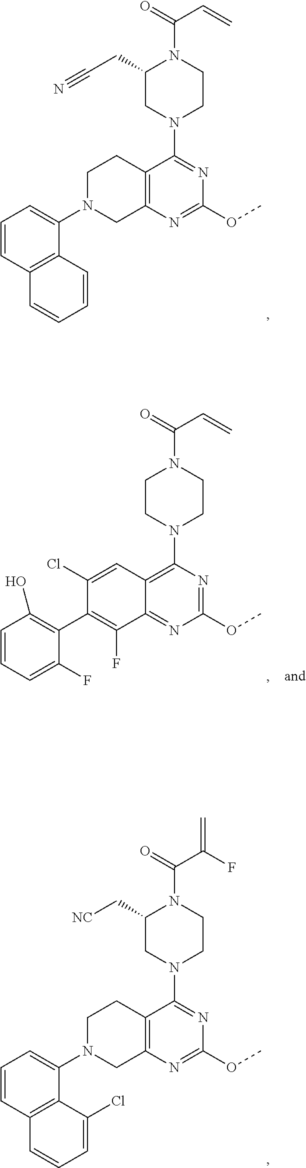

wherein * denotes an atom that is the site of attachment with the chemical linking moiety or an atom that is shared with the chemical linking moiety, and the [symbol] of the PTM is the site of attachment to the VLM or the L coupling the VLM to the PTM.

In any aspect or embodiment described herein, the PTM has a chemical structure represented by:

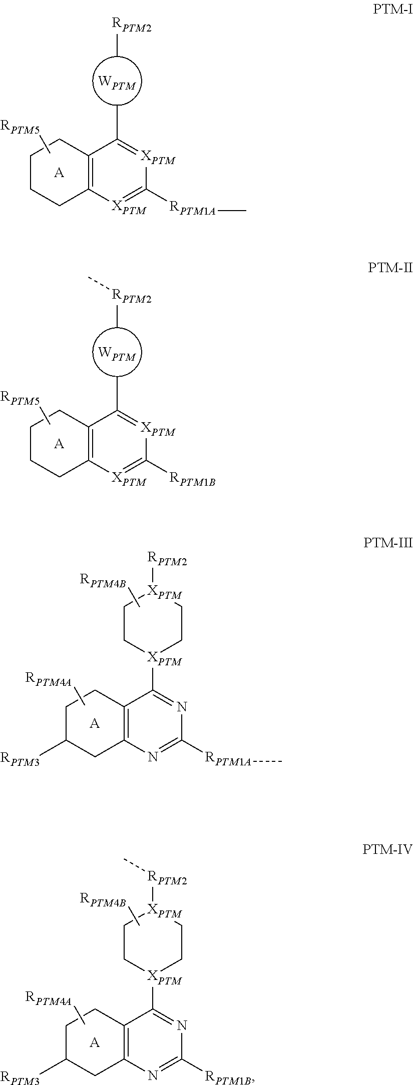

wherein:

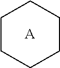

is an aryl, heteroaryl, cycloalkyl, or heterocycloalkyl;
X_{PTM} is C or N;
W_{PTM} is chosen from the group consisting of optionally substituted C₃-C₆ cycloalkyl, and optionally substituted C₃-C₆ heteroalkyl, optionally substituted C₃-C₆ heterocycloalkyl optionally substituted aryl (e.g., optionally substituted C₅-C₇ aryl), optionally substituted heteroaryl (e.g., optionally substituted C₅-C₇ heteroaryl);
R_{PTM1A} is NR_{PTM9}R_{PTM10}, OR_{PTM9}R_{PTM10}, H, optionally substituted alkyl, optionally substituted alkoxy, optionally substituted C₃-C₆ cycloalkyl, optionally substituted O—(C₃-C₆ cycloalkyl), optionally substituted C₃-C₆ heteroalkyl, optionally substituted —O—C14 alkyl-C₃₋₆ cycloalkyl, optionally substituted O—(C₃-C₆ heteroalkyl), optionally substituted O—C₁₋₄ alkyl-C₃₋₆ heteroalkyl, optionally substituted O—C₁₋₄ alkyl-C₃₋₆ heterocycloalkyl, optionally substituted aryl (e.g., optionally substituted C₅-C₇ aryl), optionally substituted O-aryl (e.g., optionally substituted O—(C₅-C₇ aryl)), optionally substituted heteroaryl (e.g., optionally substituted C₅-C₇ heteroaryl), optionally substituted O-heteroaryl (e.g., optionally substituted O—(C₅-C₇ heteroaryl)), optionally substituted

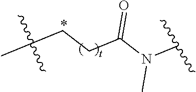

(e.g., optionally substituted with at least one alkyl, such as the * carbon may be optionally substituted with an alkyl), optionally substituted

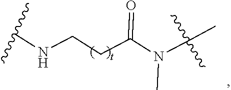

optionally substituted

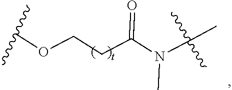

optionally substituted

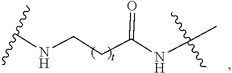

optionally substituted

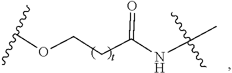

optionally substituted

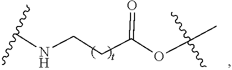

optionally substituted

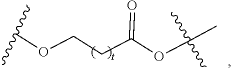

optionally substituted

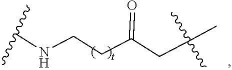

optionally substituted

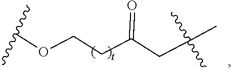

optionally substituted

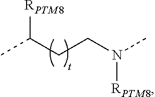

optionally substituted

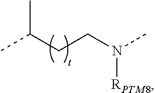

optionally substituted

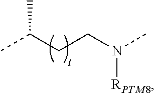

optionally substituted

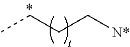

(e.g., optionally substituted with at least one alkyl, such as the * carbon may be optionally substituted with an alkyl), optionally substituted

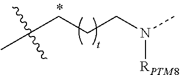

(e.g., optionally substituted with at least one alkyl, such as the * carbon may be optionally substituted with an alkyl), optionally substituted

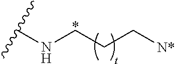

(e.g., optionally substituted with at least one alkyl, such as the * carbon may be optionally substituted with an alkyl), optionally substituted

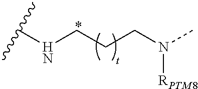

(e.g., optionally substituted with at least one alkyl, such as the * carbon may be optionally substituted with an alkyl), optionally substituted

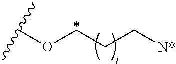

(e.g., optionally substituted with at least one alkyl, such as the * carbon may be optionally substituted with an alkyl), optionally substituted

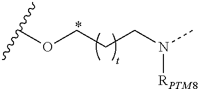

(e.g., optionally substituted with at least one alkyl, such as the * carbon may be optionally substituted with an alkyl), wherein N* is a N atom of a heterocycloalkyl (e.g., a C4-C8 heterocycloalkyl) of the linker (L);
R_{PTM1B} is NR_{PTM9}R_{PTM10}, OR_{PTM9}R_{PTM10}, H, optionally substituted alkyl, optionally substituted O-alkyl, optionally substituted C3-C6 cycloalkyl, optionally substituted O—(C3-C6 cycloalkyl), optionally substituted —O—C₁₋₄ alkyl-C₃₋₆ cycloalkyl, optionally substituted C3-C6 heteroalkyl, optionally substituted O—(C3-C6 heteroalkyl), optionally substituted O—C₁₋₄ alkyl-C₃₋₆ heteroalkyl, optionally substituted aryl (e.g., optionally substituted C5-C7 aryl), optionally substituted O-aryl (e.g., optionally substituted O—(C5-C7 aryl)), optionally substituted heteroaryl (e.g., optionally substituted C5-C7 heteroaryl), optionally substituted O-heteroaryl (e.g., optionally substituted O(C5-C7 heteroaryl)), optionally substituted

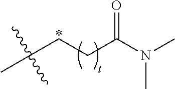

(e.g., optionally substituted with at least one alkyl, such as the * carbon may be optionally substituted with an alkyl), optionally substituted

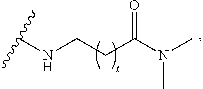

optionally substituted

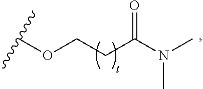

optionally substituted

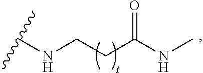

optionally substituted

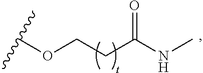

optionally substituted

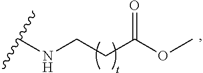

optionally substituted

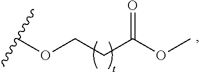

optionally substituted

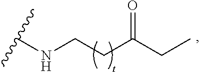

optionally substituted

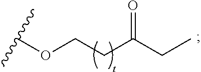

R_{PTM9} and R_{PTM10} are each independently H, optionally substituted C1-C6 alkyl, optionally substituted aliphatic amine, optionally substituted aliphatic amide;
R_{PTM2} is H, (C═O)R_{PTM2}′, optionally substituted linear or branched alkyl;
R_{PTM2′} is optionally substituted linear or branched alkyl, optionally substituted alkene, —N (R_{PTM8})₂, or —C(OH)₂;
R_{PTM3} is alkyl, alkoxy, phenyl, or napthalene, each independently substituted with OH, H, halogen;
R_{PTM4A} is OH, H, halogen, optionally substituted linear or branched C1-C6 alkyl;
R_{PTM4B} is OH, H, halogen, optionally substituted linear or branched C1-C6 alkyl;
R_{PTM5} is chosen from the group consisting of optionally substituted aryl, optionally substituted biaryl, optionally substituted heteroaryl, optionally substituted biheteroaryl, optionally substituted C3-C6 cycloalkyl, optionally substituted C3-C6 cycloheteroalkyl, halogen, H, optionally substituted linear or branched alkyl (e.g., optionally substituted linear or branched C1-C6 alkyl), OH, and alkoxy;
R_{PTM8} is a H or an alkyl (e.g., a C1 alkyl, a C2 alkyl, a C3 alkyl, or a C4 alkyl);
t is 0, 1, 2, 3, 4, 5, 6 (such as 0, 1, 2, 3); and
the [symbol] indicates the site of attachment of at least one of a linker, ULM, ULM′, VLM, VLM′, or a combination thereof.

In any aspect or embodiment described herein, the hetero-bifunctional compound is represented by the chemical structure:

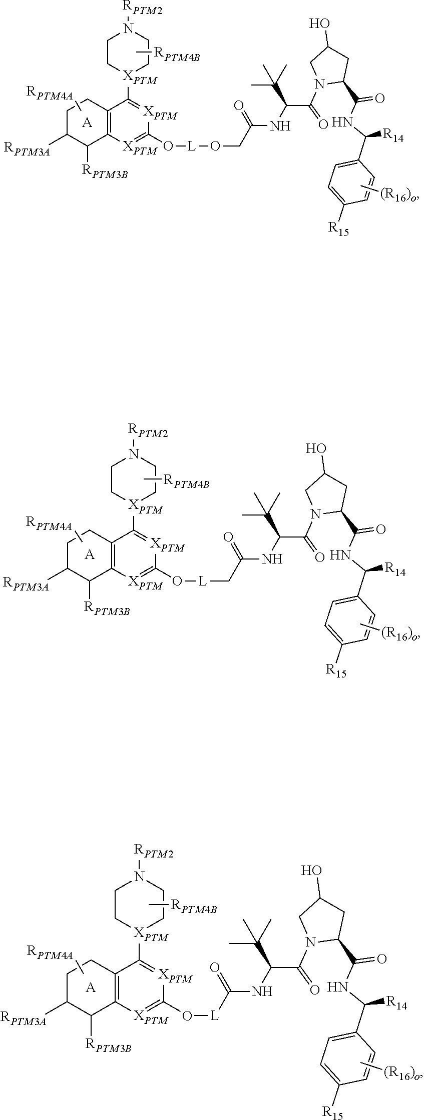

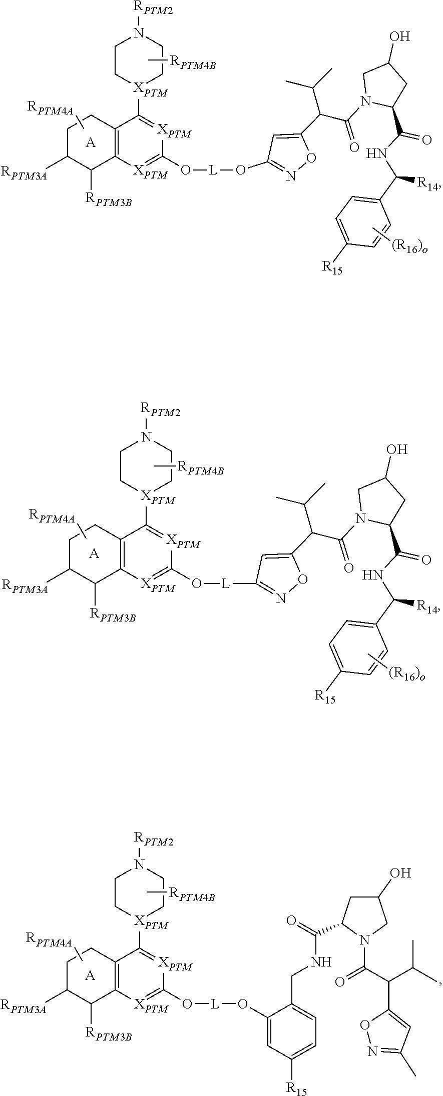

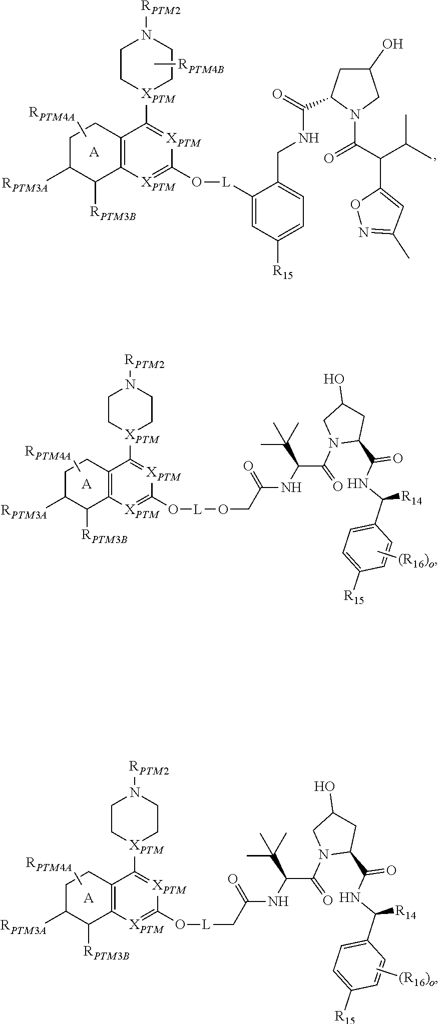

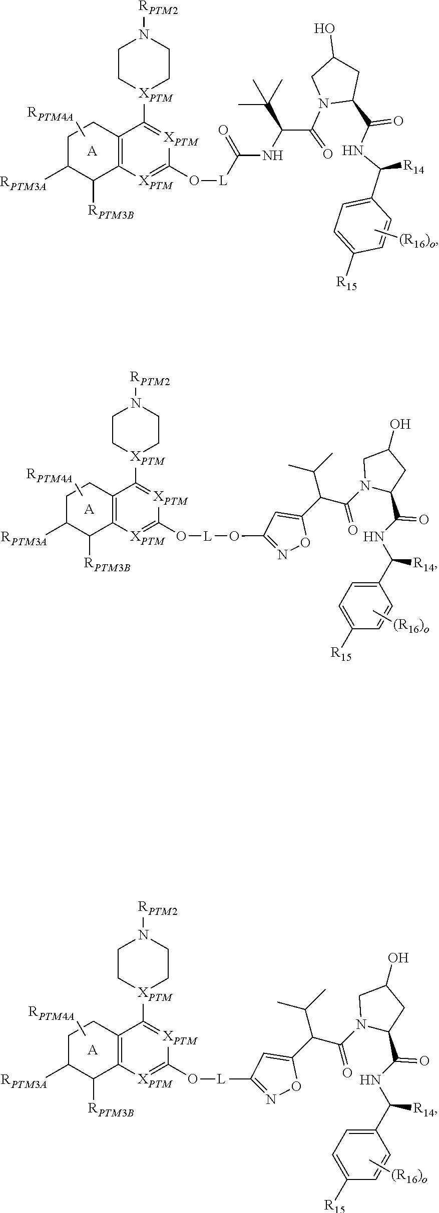

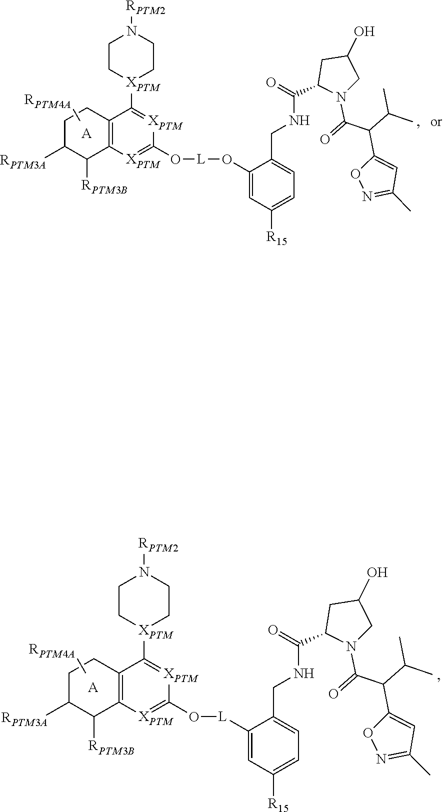

wherein:

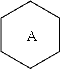

is an 6-membered aryl, 6-membered heteroaryl, or a 6-membered heterocycloalkyl, each optionally substituted with 1 or 2 halogens (e.g., Cl, F, or Br);
each X_{PTM} is individually a CH or N;
R_{PTM2} is —C(═O)C2-C4alkenyl, optionally substituted by a methyl, halogen (e.g., Cl, F, Br), amine (e.g., —NH₂, —NHCH₃, or —N(CH₃)₂), or a 3-6 membered heterocycloalkyl (e.g., a 6-membered heterocycloalkyl, a heterocycloalkyl having heteroatoms selected from O and
N, or

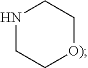

R_{PTM3A} is H, phenyl, pyridinyl, isoquinoline, or naphthalene

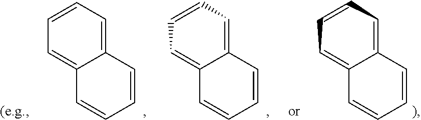

each optionally substituted by 1, 2, or 3 groups independently selected from OH, halogen (e.g., F, Cl, Br), amine (e.g., —NH₂, —NHCH₃, or —N(CH₃)₂), a linear or branched C₁-C₃ haloalkyl (e.g., a linear or branched C₁-C₃ fluoroalkyl or CF₃), —R_{PTM3C}, or a linear or branched C₁-C₃ alkyl (e.g., methyl or ethyl), wherein R_{PTM3C} is an indazole optionally substituted by 1, 2, or 3 groups independently selected from OH, halogen (e.g., F, Cl, Br), or a linear or branched C₁-C₃ alkyl (e.g., methyl or ethyl)

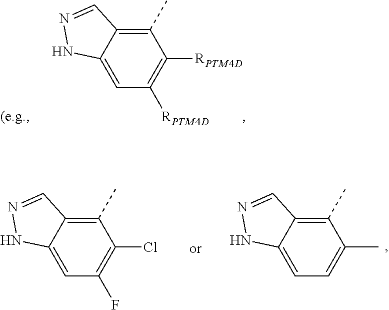

wherein each R_{PTM4D} is independently selected from a hydrogen, C₁-C₃ alkyl (e.g., methyl), or a halogen (e.g., F, Cl, Br));
R_{PTM3B} is H, halogen (e.g., Cl, F, Br), or —O—R_{PTM3C}, wherein R_{PTM3B} is optionally substituted by 1, 2, or 3 groups independently selected from OH, halogen (e.g., F, Cl, Br), or a linear or branched C₁-C₃ alkyl (e.g., methyl or ethyl)

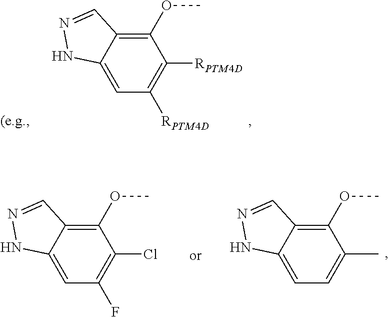

wherein each R_{PTM4D} is independently selected from a hydrogen, C₁-C₃ alkyl (e.g., methyl), or a halogen (e.g., F, Cl, Br));
R_{PTM4A} is 1 or 2 independently selected halogen (e.g., Cl, F, Br);
R_{PTM4B} is (1) —CH₂—CH₂—CN or —CH₂—CN, or (2) 1 or 2 independently selected C1-C3 alkyl (e.g., methyl or ethyl);
R₁₄ is as defined in R₁₀, R₁₄ₐ, or R_{14b} in any aspect or embodiment described herein;
R₁₅ is as defined in any aspect or embodiment described herein;
R₁₆ is as defined in any aspect or embodiment described herein; and
is as defined in any aspect or embodiment described herein.

### Therapeutic Compositions

The present invention further provides pharmaceutical compositions comprising therapeutically effective amounts of at least one bifunctional compound as described herein, in combination with a pharmaceutically acceptable carrier, additive or excipient.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions effect targeted protein degradation in a patient or subject, for example, an animal such as a human, and can be used for treating or ameliorating disease states or conditions which are modulated by degrading the target protein. In certain embodiments, the therapeutic compositions as described herein may be used to effectuate the degradation of protein for the treatment or amelioration of a KRas-related disease or disorder, e.g., accumulation or overactivity of an KRas protein or a mutated or gain-of function KRas protein, a mis-folded KRas protein, pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

In alternative aspects, the present disclosure relates to a method for treating a disease state or ameliorating one or more symptoms of a disease or condition in a subject in need thereof by degrading the KRas protein (e.g., a wildtype KRas protein or a KRas mutant protein (e.g., a gain-of-function KRas mutant protein or a KRas protein having one or more mutation selected from codon 12 missense mutation, codon 12 missense mutation, exon 2 mutation, G12V, G12C, G12D, G12A, G13D, exon 3 mutation, codon 61 missense mutation, exon 4 mutation, G12R, Q61H, G12S, A146T, G13C, Q61R, Q61L, A146V, codon 117 missense mutation, K117N, Q61K, G12F, codon 59 missense mutation, A59T, or combinations thereof) comprising administering to said patient or subject an effective amount, e.g., a therapeutically effective amount, of at least one compound as described herein, optionally in combination with a pharmaceutically acceptable carrier, additive or excipient, and optionally coadministered with an additional bioactive agent, wherein the composition is effective for treating or ameliorating the disease or disorder or one or more symptoms thereof in the subject. The method according to the present disclosure may be used to treat certain disease states, conditions or symptoms including inflammatory disease, autoimmune disease, or cancer, by virtue of the administration of effective amounts of at least one compound described herein. For example, the method according to the present disclosure may be used to treat one or more of accumulation or overactivity of an KRas protein, a mutated or gain-of function KRas protein, a mis-folded KRas protein, pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer. In any aspect or embodiment described herein, the method further comprises, prior to administering a composition or compound of the present disclosure to a subject, identifying a patient as having a mutant KRas protein (e.g., KRas^{G12C}).

The present disclosure further includes pharmaceutical compositions comprising a pharmaceutically acceptable salt, in particular, acid or base addition salts of the compounds as described herein. The acids which are used to prepare the pharmaceutically acceptable acid addition salts of the aforementioned compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds according to the present disclosure. The chemical bases that may be used as reagents to prepare pharmaceutically acceptable base salts of the present compounds are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual, intranasal, intraocular, intrathecal, vaginal, and suppository administration, among other routes of administration. The term “parenteral” as used herein includes subcutaneous, intravenous, intramuscular, intra-articular, intra-synovial, intrasternal, intrathecal, intrahepatic, intralesional and intracranial injection or infusion techniques. Enteric coated oral tablets may also be used to enhance bioavailability of the compounds from an oral route of administration. The most effective dosage form will depend upon the pharmacokinetics of the particular agent chosen as well as the type, location and severity of disease, condition or symptom, and the health of the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form or in depot formulation may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat, and combinations thereof.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch, among others known in the art. For oral administration in a capsule form, useful diluents include lactose and corn starch. When aqueous suspensions are required for oral use, the active ingredient may be combined with emulsifying and suspending agents. If desired, certain sweetening, flavoring or coloring agents may also be added. Lubricating agents, such as magnesium stearate, are also typically added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. For topical applications, the pharmaceutical composition can be formulated in a transdermal patch, which can either be a reservoir patch or a matrix patch comprising the active compound combined with one or more carriers, buffers, absorption enhancers, and providing from 1 day to two weeks of continuous administration.

Alternatively, the pharmaceutical compositions of the present disclosure may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water.

Alternatively, the pharmaceutical compositions of the present disclosure can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Alternatively, the pharmaceutical compositions of the present disclosure can be formulated for ophthalmic use. For example, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active pharmaceutical ingredient in a pharmaceutical composition as described herein that may be combined with the carrier materials to produce a single dosage form will vary depending upon the condition of the subject and disease, condition or symptom treated, the particular mode of administration, and the condition of the subject. Preferably, the compositions should be formulated to contain between about 0.05 milligram and about 750 milligrams or more, more preferably about 1 milligram to about 600 milligrams, and even more preferably about 10 milligrams to about 500 milligrams of active ingredient, alone or in combination with another compound according to the present disclosure.

It should also be understood that a specific dosage and treatment regimen for any particular patient will depend upon a variety of factors, including the activity and bioavailability of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure depending upon the pharmaceutically acceptable salt or solvate thereof, optionally in a pharmaceutically acceptable carrier or diluent, either alone, or in combination with another known therapeutic agent. In any aspect or embodiment described herein, the method further includes, prior to administering a composition or compound of the present disclosure to a subject, identifying a patient as having a mutant KRas protein (e.g., KRas^{G12C}).

In certain aspects, the active compound is combined with the pharmaceutically acceptable carrier or diluent in an amount sufficient to deliver to a patient a therapeutically effective amount for the desired indication, without causing an undue degree of serious toxic effects in the patient treated. A preferred dose of the active compound for all of the herein-mentioned conditions is in the range from about 10 nanograms per kilograms (ng/kg) to 300 milligrams per kilograms (mg/kg), preferably 0.1 to 100 mg/kg per day, more generally 0.5 to about 25 mg per kilogram body weight of the recipient/patient per day. A typical topical dosage will range from 0.01-5% wt/wt in a suitable carrier.

In certain aspects, the compound is conveniently administered in any suitable unit dosage form, including but not limited to a dosage form containing less than 1 milligrams (mg), 1 mg to 3000 mg, or 5 mg to 500 mg of active ingredient per unit dosage form. An oral dosage of about 25 mg-250 mg is often convenient.

In certain aspects, the active ingredient is preferably administered to achieve peak plasma concentrations of the active compound of about 0.00001-30 millimole (mM), preferably about 0.1-30 micromole (μM). This may be achieved, for example, by the intravenous injection of a solution or formulation of the active ingredient, optionally in saline, or an aqueous medium or administered as a bolus of the active ingredient. Oral administration may also be appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, as described herein among others. In certain preferred aspects of the disclosure, one or more compounds according to the present disclosure are coadministered with another bioactive agent, such as an anti-cancer agent or a wound healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In any aspect or embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

Therapeutic Methods

In an additional aspect, the description provides therapeutic methods comprising administration of an effective amount of a compound as described herein or salt form thereof, and a pharmaceutically acceptable carrier. The therapeutic methods are useful to effect protein degradation in a patient or subject in need thereof, for example, an animal such as a human, for treating or ameliorating a disease state, condition or related symptom that may be treated through targeted protein degradation.

The terms “treat”, “treating”, and “treatment”, etc., as used herein, refer to any action providing a benefit to a patient for which the present compounds may be administered, including the treatment of any disease state, condition, or symptom which is related to the protein to which the present compounds bind. Disease states or conditions, including cancer, which may be treated using compounds according to the present disclosure are set forth hereinabove.

The description provides therapeutic methods for effectuating the degradation of proteins of interest for the treatment or amelioration of a disease, e.g., pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer. As such, in another aspect, the description provides a method of ubiquitinating/degrading a target protein in a cell. In certain embodiments, the method comprises administering a bifunctional compound of the invention. The control or reduction of specific protein levels in cells of a subject as afforded by the present disclosure provides treatment of a disease state, condition, or symptom. In any aspect or embodiment, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In any aspect or embodiment described herein, the method further includes, prior to administering a composition or compound of the present disclosure to a subject, identifying a patient as having a mutant KRas protein (e.g., KRas^{G12C}).

In another aspect, the description provides methods for identifying the effects of the degradation of proteins of interest in a biological system using compounds according to the present disclosure.

In another aspect, the description provides a process for making a molecule that can cause degradation of KRas in a cell (e.g., in vivo or in vitro), comprising the steps of: (i) providing a small molecule that binds to the KRas or a mutated form thereof; (ii) providing an E3 ubiquitin ligase binding moiety (ULM), preferably a VLM as described herein; and (iii) covalently coupling the small molecule of step (i) to the ULM of step (ii) via a chemical linking group (L) to form a compound which binds to both a VHL E3 ubiquitin ligase and KRas protein and/or mutated form in the cell, such that the VHL E3 ubiquitin ligase is in proximity to, and ubiquitinates the KRas protein bound thereto, such that the ubiquitinated KRas is then degraded.

In another aspect, the description provides a method for detecting whether a molecule can trigger degradation of a KRas protein in a cell (e.g., in vivo or in vitro), the method comprising the steps of: (i) providing a molecule for which the ability to trigger degradation of KRas protein in a cell is to be detected, said molecule comprising the structure: VLM-L-PTM, wherein VLM is a VHL E3 ubiquitin ligase binding moiety capable of binding a VHL E3 ubiquitin ligase in a cell, which VLM is as described herein, such a derivative of trans-3-hydroxyproline, where both nitrogen and carboxylic acid in trans-3-hydroxyproline are functionalized as amides; PTM is a protein targeting moiety, which is a small molecule that binds to KRas and/or mutated KRas form thereof, said KRas having at least one lysine residue available to be ubiquitinated by a VHL E3 ubiquitin ligase bound to the VLM of the molecule; and L is a chemical linking group that covalently links the VLM to the PTM to form the molecule; (ii) incubating a KRas protein-expressing cell in the presence of the molecule of step (i); and (iii) detecting whether the KRas protein in the cell has been degraded.

In any of the aspects or embodiments described herein, the small molecule capable of binding KRas, is a small molecule that binds of KRas. In certain embodiments, the small molecule that binds the KRas is as described herein.

In another aspect of said treatment, the present disclosure provides a method of treating a human patient in need of said treatment of a disease state, condition, or symptom causally related to KRas, and/or KRas mutated form, expression, over-expression, mutation, aggregation, accumulation, misfolding or dysregulation where the degradation of the KRas protein will produce a therapeutic effect in the patient, the method comprising administering to the patient an effective amount of a compound according to the present disclosure, optionally in combination with another bioactive agent.

The disease state, condition, or symptom may be caused by a microbial agent or other exogenous agent such as a virus, bacteria, fungus, protozoa or other microbe, or may be a disease state, which is caused by expression, overexpression, mutation, misfolding, or dysregulation of the protein, which leads to a disease state, condition, or symptom.

In another aspect, the present disclosure provides a method of treating or ameliorating at least one symptom of a disease or condition in a subject, comprising the steps of: providing a subject identified as having a symptom of a disease or condition causally related to expression, overexpression, mutation, misfolding, or dysregulation of KRas protein and/or mutated form thereof in the subject, and the symptom of the disease or condition is treated or ameliorated by degrading KRas protein and/or mutated form thereof in cells of the subject; and administering to the subject therapeutically effective amount of a compound comprising a small molecule of the present invention such that the KRas protein and/or mutated form thereof is degraded, thereby treating or ameliorating at least one symptom of a disease or condition in the subject.

The term “disease state or condition” is used to describe any disease state or condition wherein protein expression, overexpression, mutation, misfolding, or dysregulation (e.g., the amount of protein expressed in a patient is elevated) occurs and where degradation of the KRas protein and/or mutated form thereof to reduce or stabilize the level of KRas protein (whether mutated or not) in a patient provides beneficial therapy or relief of symptoms to a patient in need thereof. In certain instances, the disease state, condition, or symptom may be cured.

Disease state, condition, or symptom which may be treated using compounds according to the present disclosure include, for example, pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

The term “bioactive agent” is used to describe an agent, other than a compound according to the present disclosure, which is used in combination with a present compound as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-autoimmune disease agent” is used to describe an anti-autoimmune disease therapeutic agent, which may be combined with a compound according to the present disclosure to treat autoimmune disease. These agents include, for example, infliximab, tofacitinib, baricitinib, secukinumab, adalimumab, etanercept, golimumab, certolizumab pepol, anti-proliferative drugs (for example, mycophenolate mofetil) and corticosteroids.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### Examples

### Abbreviations

ACN Acetonitrile

AcOH Acetic acid

Boc tert-butoxycarbonyl

dba Dibenzylideneacetone

DBU 1,8-Diazabicyclo[5.4.0]undec-7-ene

DCM Dichloromethane

DMA Dimethylacetamide

DME Dimethoxyethane

DMF Dimethylformamide

DMSO Dimethyl Sulfoxide

DMAC/DMA Dimethylacetamide

DIEA N,N-Diisopropylethylamine

EDTA Ethylenediaminetetraacetic acid

EtOAc/EA Ethyl Acetate

EtOH Ethanol

FA Formic Acid

HPLC High pressure liquid chromatography

Hz Hertz

IBX 2-Iodoxybenzoic acid

LAH Lithium aluminium hydride

LCMS Liquid Chromatography/Mass Spectrometry

LiHMDS Lithium bis(trimethylsilyl)amide

MHz Megahertz

NBS N-Bromosuccinimide

NCS N-Chlorosuccinimide

NMR Nuclear Magnetic Resonance

NMP N-Methyl-2-pyrrolidone

MeOH Methanol

MPLC Medium pressure liquid chromatography

MTBE Methyl tert-butyl ether

PE Petroleum ether

Psi Pound-force per square inch

RT or r.t. Room temperature

SFC Supercritical fluid chromatography

TEA Triethylamine

THF Tetrahydrofuran

TFA Trifluoracetic acid

TLC Thin layer chromatography

TMS Trimethylsilyl

General Synthetic Approach

The synthetic realization and optimization of the bifunctional molecules as described herein may be approached in a stepwise or modular fashion. For example, identification of compounds that bind to the target protein, i.e., KRas can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the chemical linking group previously referred to herein. Where crystallographic or NMR structural data are available, these can be used to focus such a synthetic effort.

In a very analogous way one can identify and optimize ligands for an E3 Ligase.

With PTMs and ULMs (e.g. VLMs) in hand, one skilled in the art can use known synthetic methods for their combination with or without a chemical linking group(s). Chemical linking group(s) can be synthesized with a range of compositions, lengths and flexibility and functionalized such that the PTM and ULM groups can be attached sequentially to distal ends of the linker. Thus, a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

Synthetic Procedures

General Synthetic Scheme

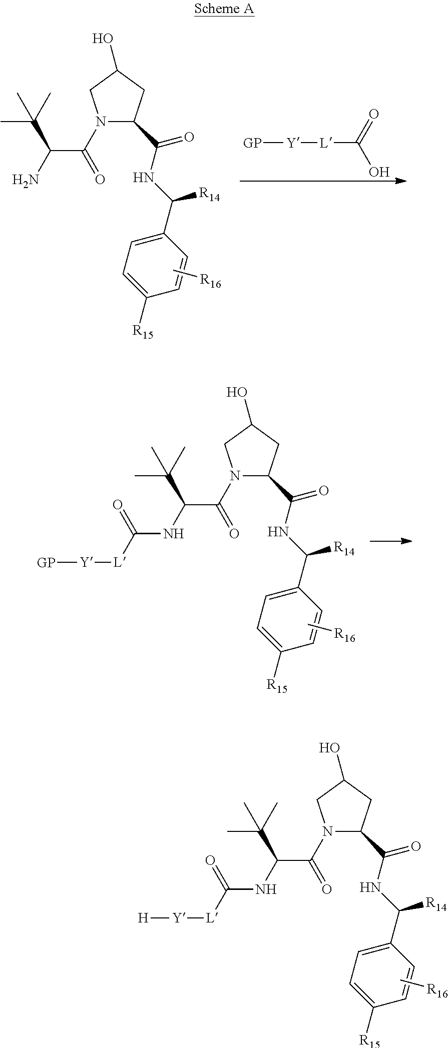

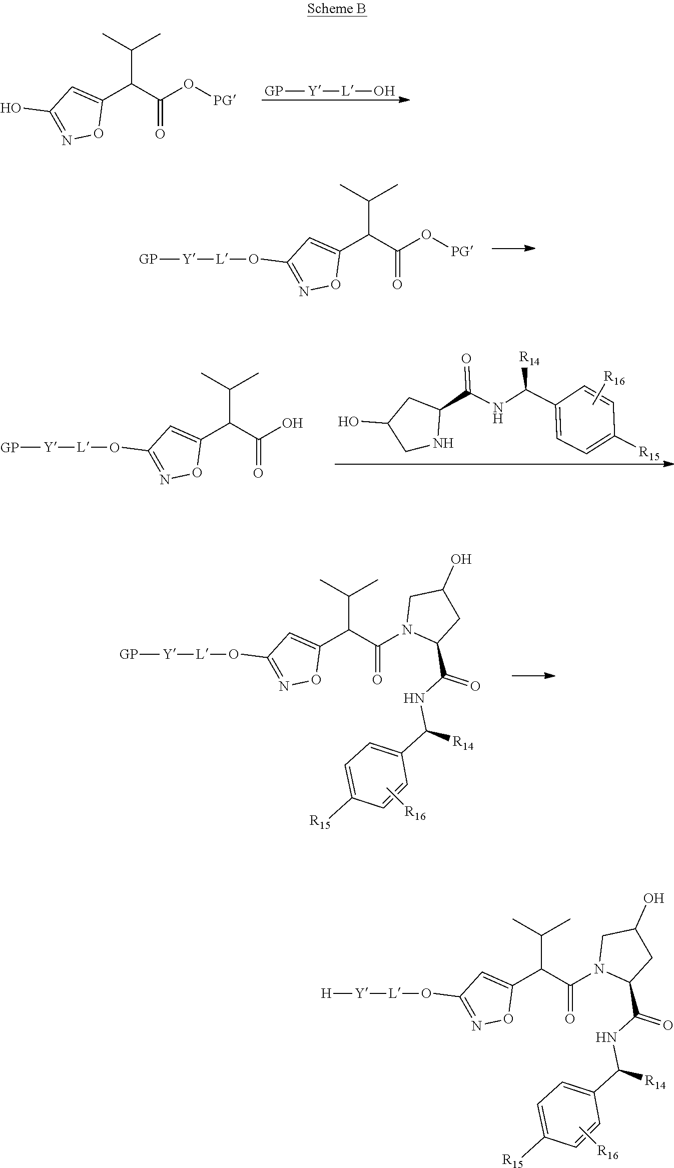

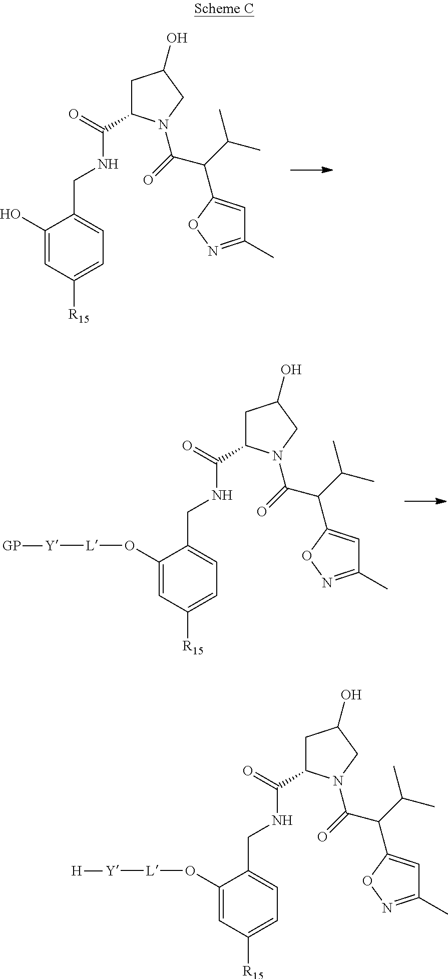

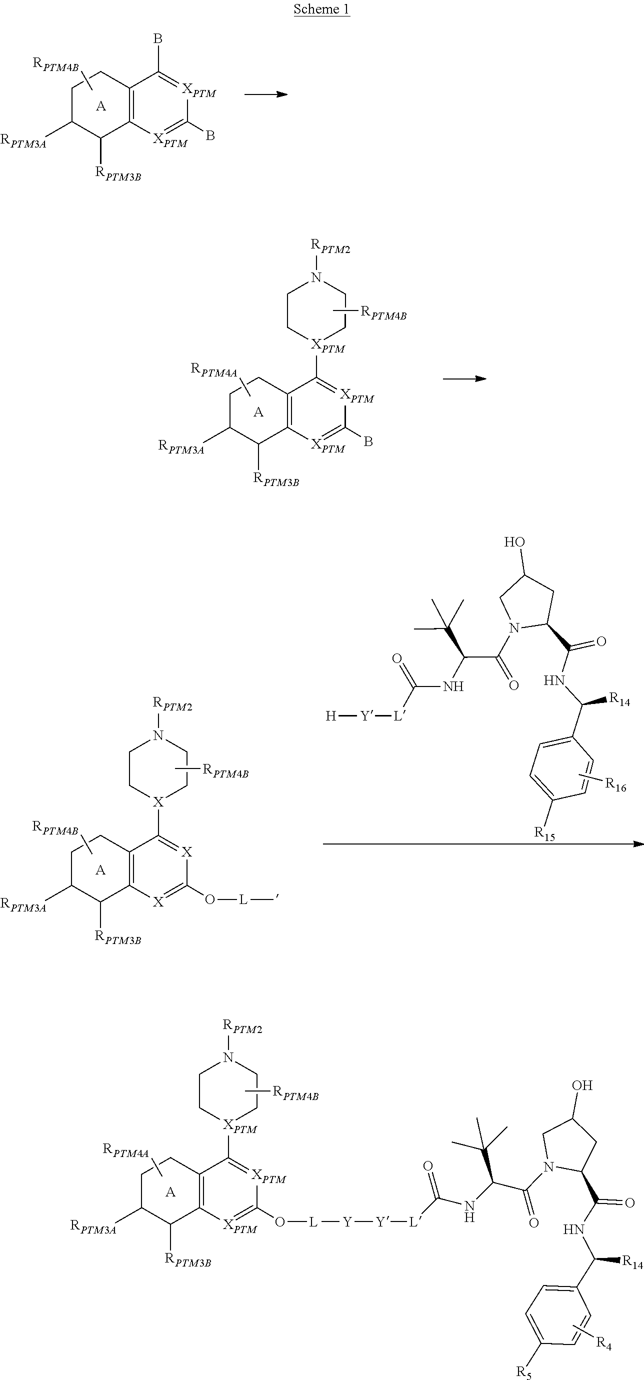

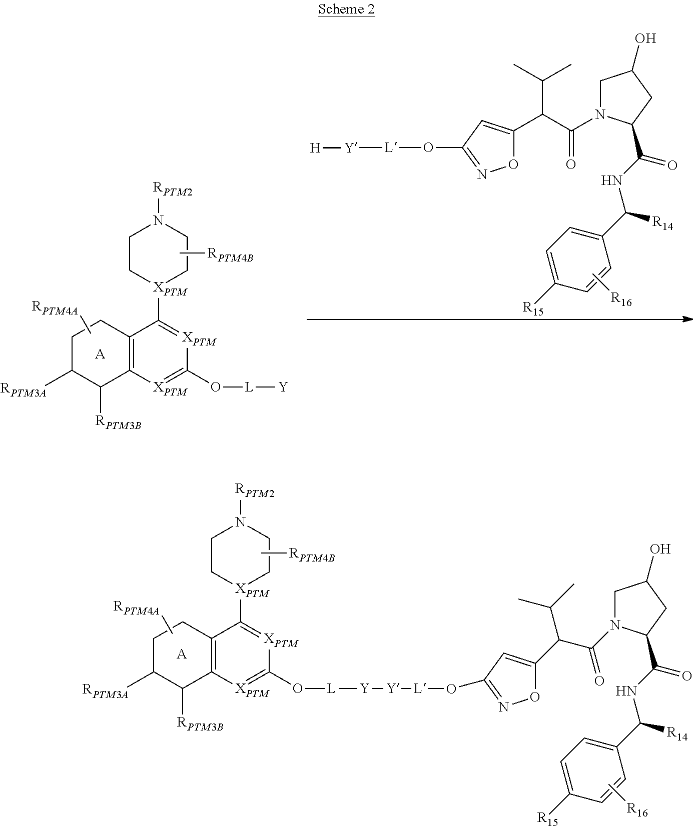

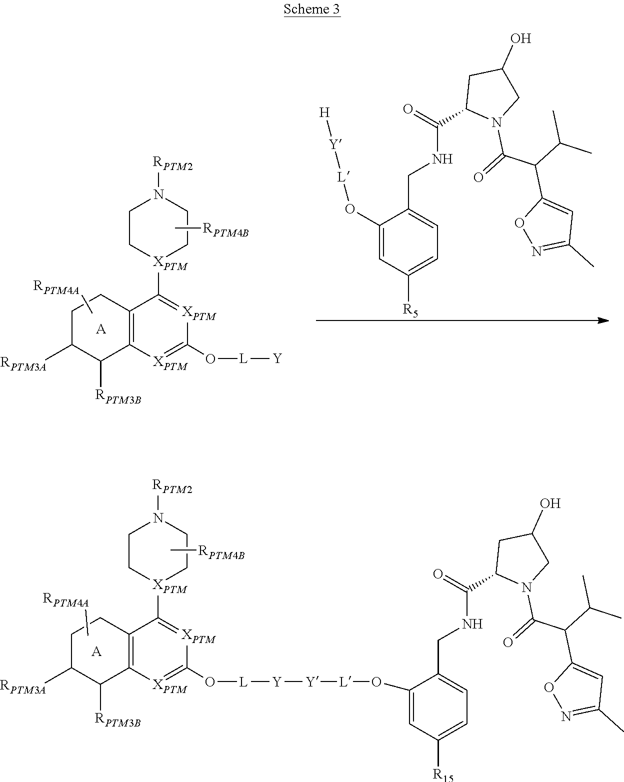

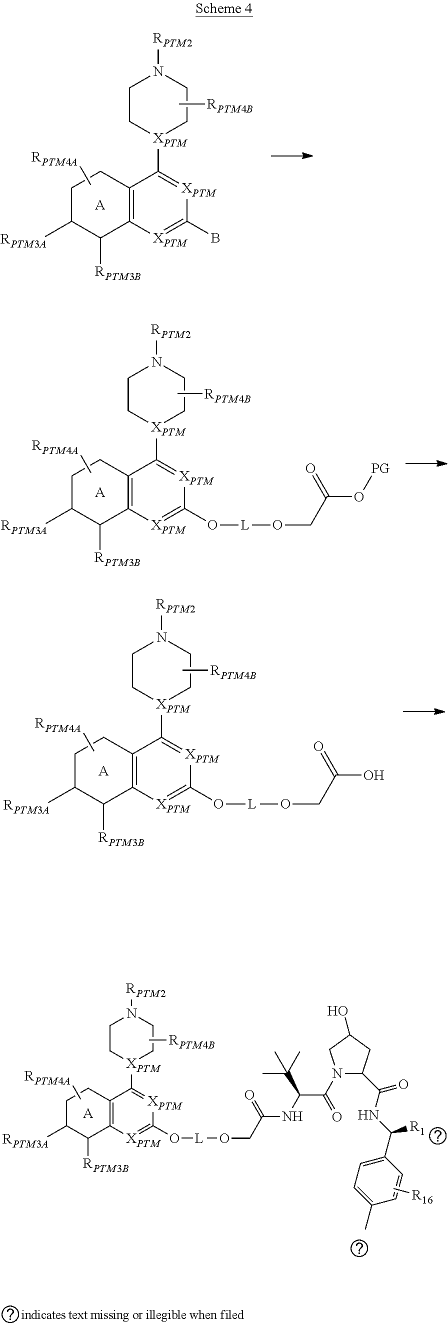

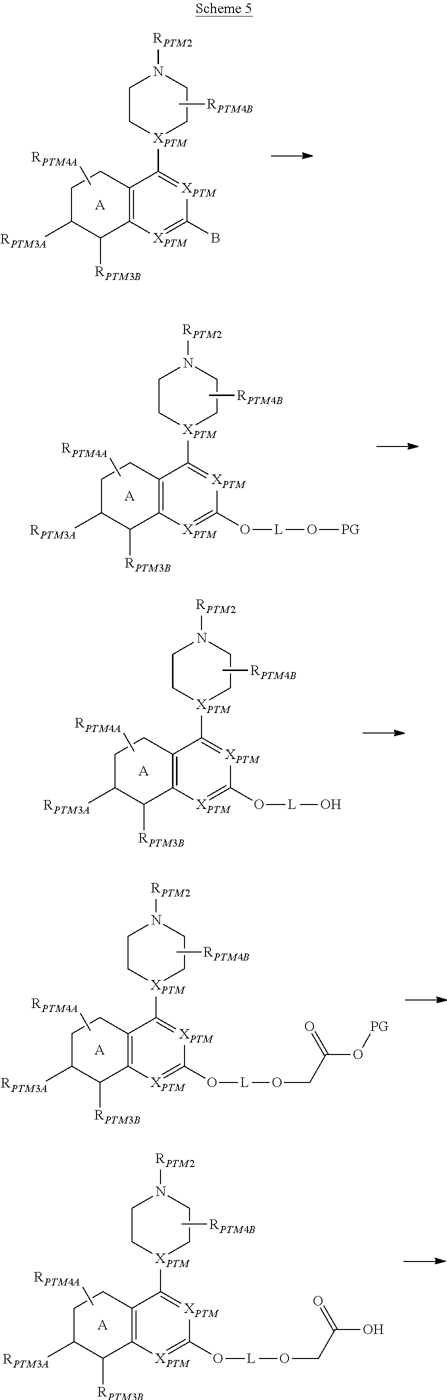

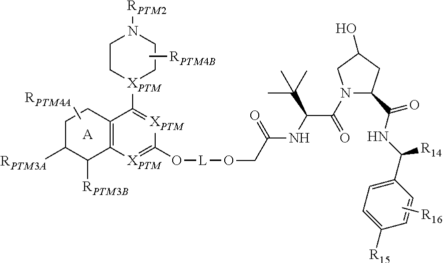

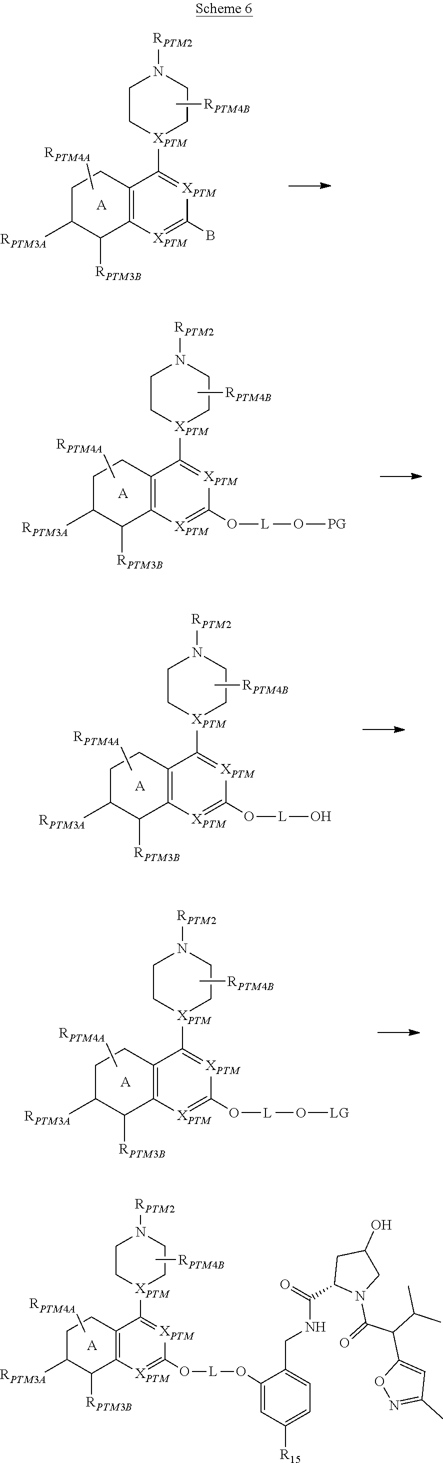

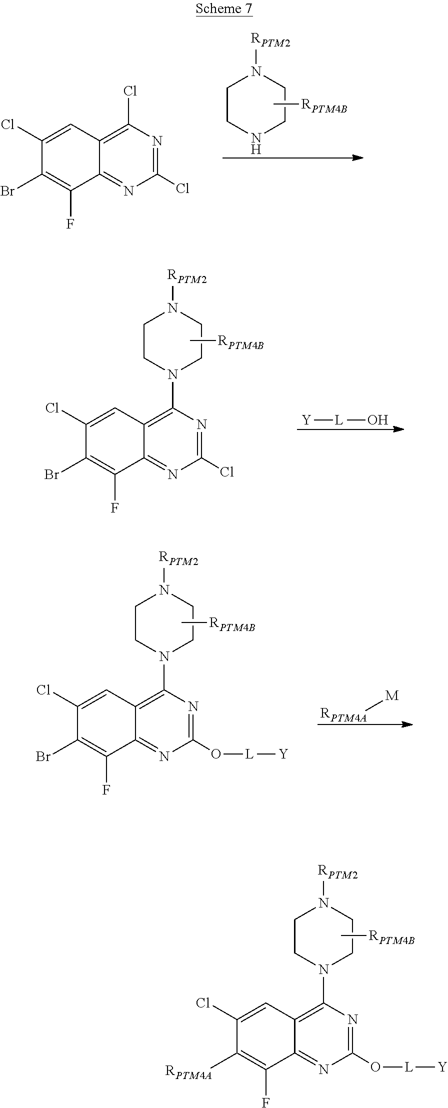

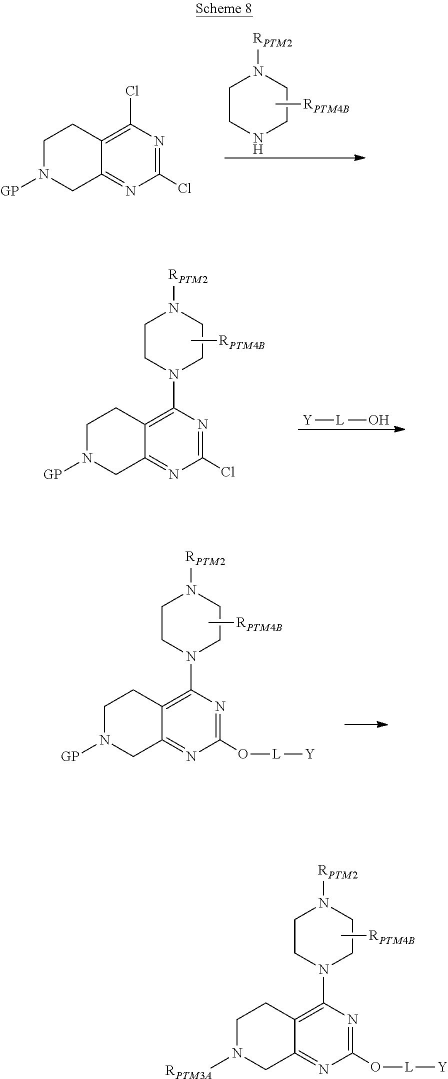

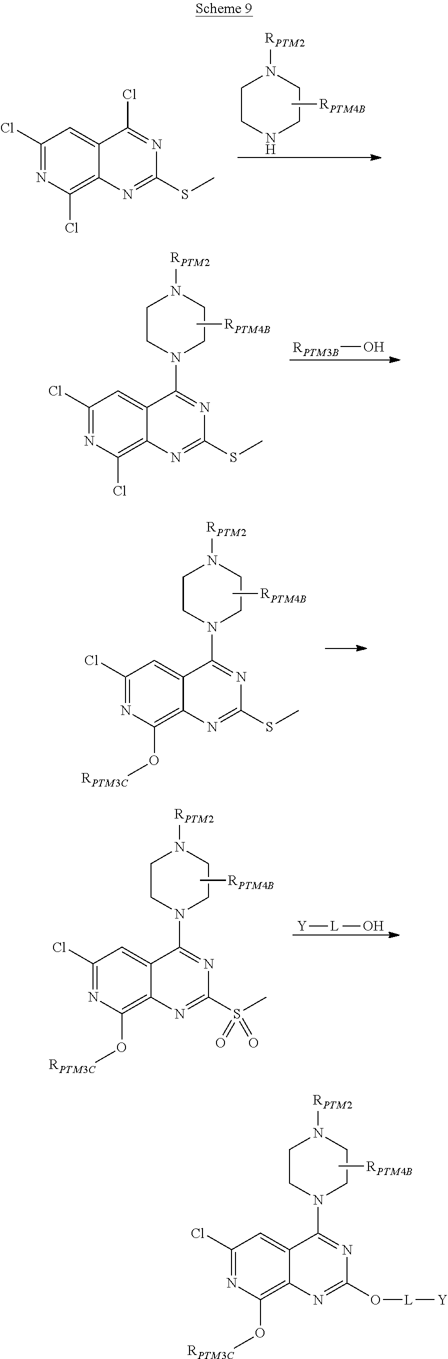

### Exemplary Synthesis of tert-butyl (2S)-4-[6-chloro-2-(2,2-dimethoxyethoxy)-8-fluoro-7-(3-hydroxy-1-naphthyl)quinazolin-4-yl]-2-(cyanomethyl)piperazine-1-carboxylate

### Step 1: Preparation of 1-benzyl 4-(tert-butyl) (R)-2-(hydroxymethyl)piperazine-1,4-dicarboxylate

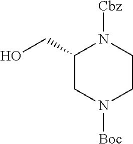 [SMILES: CC(C)(C)OC(=O)N1CCN(C(=O)OCc2ccccc2)[C@@H](CO)C1]

To a solution of tert-butyl (3R)-3-(hydroxymethyl)piperazine-1-carboxylate (10 g, 46.24 mmol, 1 e) in EtOAc (50 mL) and H₂O (50 mL) was added NaHCO₃ (11.66 g, 138.80 mmol, 5.40 mL, 3.00 eq) in one portion followed by CbzCl (11.88 g, 69.64 mmol, 9.90 mL, 1.51 eq), and the resulting mixture was stirred at 0° C. for 30 minutes, then at 10° C. for 5 hours. The organic layer was separated and washed with water (10 mL). The aqueous phase was extracted with EtOAc (100 mL). The organic layers were combined, washed with water (3×30 mL), brine (30 mL), dried over Na₂SO₄, and concentrated under reduced pressure. The resulting yellow liquid was purified by SiO₂ column chromatography (25-50 EtOAc in petroleum ether) to obtain the desired product tert-butyl (2S)-4-(6-chloro-8-fluoro-7-(3-hydroxynaphthalen-1-yl)-2-(2-oxoethoxy)quinazolin-4-yl)-2-(cyanomethyl)piperazine-1-carboxylate (15 g, 36.81 mmol, 80% yield, 86% purity) as colorless liquid. LC/LC/MS (ESI) m/z: 373.1 [M+Na]⁺.

### Step 2: Preparation of 1-benzyl 4-(tert-butyl) (R)-2-(((methylsulfonyl)oxy)methyl)piperazine-1,4-dicarboxylate

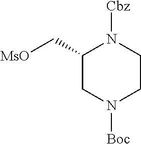 [SMILES: CC(C)(C)OC(=O)N1CCN(C(=O)OCc2ccccc2)[C@@H](COS(C)(=O)=O)C1]

To a solution of tert-butyl (2S)-4-(6-chloro-8-fluoro-7-(3-hydroxynaphthalen-1-yl)-2-(2-oxoethoxy)quinazolin-4-yl)-2-(cyanomethyl)piperazine-1-carboxylate (60 g, 171.23 mmol, 1 eq) in CH₂Cl₂ (500 mL) was added triethylamine (51.98 g, 513.69 mmol, 71.50 mL, 3 eq) in one portion. Methanesulfonyl chloride (29.42 g, 256.84 mmol, 19.88 mL, 1.5 eq) was added slowly to the solution for 30 minutes at 0° C., and the resulting mixture was stirred at 25° C. for 2 hours. The reaction was poured onto water (500 mL), and the resulting mixture was extracted with EtOAc (2×600 mL). The combined organic extracts were washed sequentially with saturated aqueous NH₄Cl (500 mL), saturated aqueous NaHCO₃ (500 mL), and brine, and then concentrated under reduced pressure to afford the crude product 1-benzyl 4-(tert-butyl) (R)-2-(((methylsulfonyl)oxy)methyl)piperazine-1,4-dicarboxylate (70 g) as a yellow liquid. ¹H-NMR (400 MHz, CDCl₃) δ 7.42-7.34 (m, 5H), 5.17 (s, 2H), 4.50-4.42 (m, 1H), 4.25-4.15 (m, 2H), 4.10-3.93 (m, 3H), 3.08-3.06 (m, 2H), 2.94-2.92 (m, 4H), 1.48 (s, 9H).

### Step 3: Preparation of 1-benzyl 4-(tert-butyl) (S)-2-(cyanomethyl)piperazine-1,4-dicarboxylate

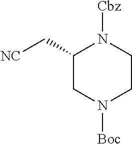 [SMILES: CC(C)(C)OC(=O)N1CCN(C(=O)OCc2ccccc2)[C@@H](CC#N)C1]

To a solution 1-benzyl 4-(tert-butyl) (R)-2-(((methylsulfonyl)oxy)methyl)piperazine-1,4-dicarboxylate (70 g, 163.36 mmol, 1 eq) in DMA (5 mL) was added KCN (16.06 g, 246.68 mmol, 10.57 mL, 1.51 eq) in one portion, and the reaction mixture was stirred at 90° C. for 8 hours. The reaction was poured onto EtOAc (2 L), and the resulting the mixture was washed with H₂O (2×500 mL). The organic layer was washed with brine (50 mL), and then concentrated under reduced pressure. The resulting yellow liquid was purified by SiO₂ column chromatography (10-20% EtOAc in petroleum ether), to afford the desired product 1-benzyl 4-(tert-butyl) (S)-2-(cyanomethyl)piperazine-1,4-dicarboxylate (28 g, 77.90 mmol, 47.69% yield) as yellow liquid. LC/MS (ESI) m/z: 260.2 [M-Boc+H]⁺.

### Step 4: Preparation of (S)-2-(piperazin-2-yl)acetonitrile

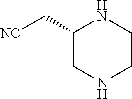

To a solution of 1-benzyl 4-(tert-butyl) (S)-2-(cyanomethyl)piperazine-1,4-dicarboxylate (6.5 g, 25.07 mmol, 1 eq) in CH₃OH (10 mL) were added NH₄OH (4.84 g, 41.47 mmol, 5.32 mL, 30% purity, 1.65 eq) and Pd/C (1 g, 10% purity) in one portion under N₂. The suspension was degassed under vacuum, purged with H₂ several times, and then stirred under H₂ (50.53 mg, 25.07 mmol, 1 eq, 15 psi) at 10° C. for 1 hour. The suspension was filtered, and the resulting clear solution was concentrated under reduced pressure. The resulting yellow liquid was reacted with HCl (4N in dioxane, 40.0 mL) and then concentrated to afford the crude product (S)-2-(piperazin-2-yl)acetonitrile (3 g, 23.97 mmol, 95.61% yield) as yellow liquid.

### Step 5: Preparation of tert-butyl (2S)-4-(7-bromo-2,6-dichloro-8-fluoro-quinazolin-4-yl)-2-(cyanomethyl)piperazine-1-carboxylate

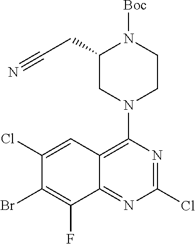 [SMILES: CC(C)(C)OC(=O)N1CCN(c2nc(Cl)nc3c(F)c(Br)c(Cl)cc23)C[C@@H]1CC#N]

To a solution of 7-bromo-2,4,6-trichloro-8-fluoro-quinazoline (7.50 g, 22.69 mmol, 1 eq) and diisopropylethylamine (17.59 g, 136.13 mmol, 23.71 mL, 6 eq) in CH₂Cl₂ (50 mL) was added (S)-2-(piperazin-2-yl)acetonitrile (2.84 g, 22.69 mmol, 1 eq) in CH₂Cl₂ (10 mL) dropwise for 20 minutes, and the reaction mixture was stirred at 0° C. for 30 minutes. (Boc)₂O (9.91 g, 45.39 mmol, 10.43 mL, 2 eq) was then added in portions over 10 minutes, and the resulting mixture was stirred at 0° C. for 30 minutes. The solution was concentrated under reduced pressure, and the resulting yellow solid was purified SiO₂ by column chromatography (0-25% EtOAc in petroleum ether) to afford the desired product tert-butyl (2S)-4-(7-bromo-2,6-dichloro-8-fluoro-quinazolin-4-yl)-2-(cyanomethyl)piperazine-1-carboxylate (8.1 g, 15.60 mmol, 68.75% yield) as a yellow solid. LC/MS (ESI) m/z: 520.1 [M+H]⁺.

### Step 6: Preparation of tert-butyl (2S)-4-[7-bromo-6-chloro-2-(2,2-dimethoxyethoxy)-8-fluoro-quinazolin-4-yl]-2-(cyanomethyl)piperazine-1-carboxylate

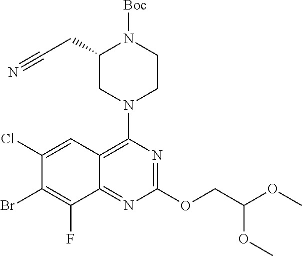 [SMILES: COC(COc1nc(N2CCN(C(=O)OC(C)(C)C)[C@@H](CC#N)C2)c2cc(Cl)c(Br)c(F)c2n1)OC]

To a solution of tert-butyl (2S)-4-(7-bromo-2,6-dichloro-8-fluoro-quinazolin-4-yl)-2-(cyanomethyl)piperazine-1-carboxylate (6 g, 11.56 mmol, 1 eq) and 2,2-dimethoxyethanol (2.45 g, 23.11 mmol, 2 eq) in CH₃CN (50 mL) were added DABCO (129.63 mg, 1.16 mmol, 127.09 uL, 0.1 eq) and Cs₂CO₃ (4.89 g, 15.02 mmol, 1.3 eq) in one portion, and the reaction mixture was stirred at 50° C. for 2 hours. The suspension was filtered, and the resulting clear yellow solution was concentrated under reduced pressure to give a yellow solid. Purification by SiO₂ column chromatography (10-24% EtOAc in petroleum ether) afforded tert-butyl (2S)-4-[7-bromo-6-chloro-2-(2,2-dimethoxyethoxy)-8-fluoro-quinazolin-4-yl]-2-(cyanomethyl)piperazine-1-carboxylate (6.4 g, 10.87 mmol, 94.05% yield) as a yellow solid.

### Step 7: Preparation of tert-butyl (2S)-4-[6-chloro-2-(2,2-dimethoxyethoxy)-8-fluoro-7-(3-hydroxy-1-naphthyl)quinazolin-4-yl]-2-(cyanomethyl)piperazine-1-carboxylate

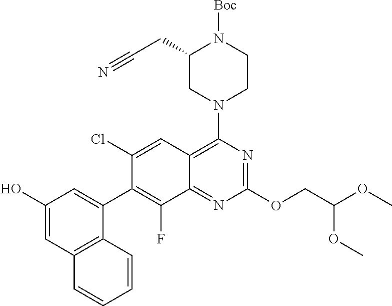 [SMILES: COC(COc1nc(N2CCN(C(=O)OC(C)(C)C)[C@@H](CC#N)C2)c2cc(Cl)c(-c3cc(O)cc4ccccc34)c(F)c2n1)OC]

A mixture of 4-(4,4,5,5-tetramethyl-1,3,2-dioxaborolan-2-yl)naphthalen-2-ol (2.94 g, 10.87 mmol, 286.80 uL, 1 eq), tert-butyl (2S)-4-[7-bromo-6-chloro-2-(2,2-dimethoxyethoxy)-8-fluoro-quinazolin-4-yl]-2-(cyanomethyl)piperazine-1-carboxylate (6.4 g, 10.87 mmol, 1 eq), K₃PO4 (1.5 M, 21.74 mL, 3 eq), and XPhos Pd G3 (459.99 mg, 543.43 umol, 0.05 eq) in THF (15 mL) was degassed and then heated at 50° C. for 1 hour under N₂ atmosphere. The resulting suspension was filtered, and the yellow filtrate was concentrated under reduced pressure to give a yellow solid. Purification by prep-TLC(SiO₂, 50% EtOAc/petroleum ether) afforded tert-butyl (2S)-4-[6-chloro-2-(2,2-dimethoxyethoxy)-8-fluoro-7-(3-hydroxy-1-naphthyl)quinazolin-4-yl]-2-(cyanomethyl)piperazine-1-carboxylate (6.3 g, 9.66 mmol, 88.89% yield) as a yellow solid. LC/MS (ESI) m/z: 652.3 [M+H]⁺.

### Exemplary Synthesis of tert-butyl (2S)-4-[6-chloro-8-fluoro-7-(3-hydroxy-1-naphthyl)-2-[(1R)-1-methyl-2-oxo-ethoxy]quinazolin-4-yl]-2-(cyanomethyl)piperazine-1-carboxylate

### Step 1: Preparation of methyl (R)-2-(benzyloxy)propanoate

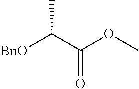

To solution of (2R)-2-benzyloxypropanoic acid (20.00 g, 110.99 mmol, 1.00 eq) in methanol (150 mL) was cooled to 0° C., then sulfurous dichloride (39.61 g, 332.96 mmol, 24.2 mL, 3.00 eq) was added dropwise. The mixture was then stirred at 50° C. for 4 hours. The reaction mixture concentrated under reduced pressure to give a residue. The residue was diluted with saturated sodium bicarbonate solution (200 mL), then extracted with ethyl acetate (200 mL×2). The combined organic layers were dried over anhydrous sodium sulfate, filtered and concentrated under reduced pressure to give the product, methyl (2R)-2-benzyloxypropanoate (21.96 g) as a yellow oil. LC/MS (ESI) m/z: 217.1 [M+23]⁺; ¹H-NMR (400 MHz, CDCl₃) δ 7.40-7.27 (m, 5H), 4.70 (d, J=11.6 Hz, 1H), 4.46 (d, J=11.6 Hz, 1H), 4.08 (q, J=6.8 Hz, 1H), 3.77 (s, 3H), 1.45 (d, J=6.8 Hz, 3H).

### Step 2: Preparation of (R)-2-(benzyloxy)propanal

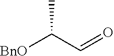

A solution of methyl (2R)-2-benzyloxypropanoate (20.96 g, 107.92 mmol, 1.00 eq) in dichloromethane (200 mL) was cooled to −78° C., then diisobutylaluminum hydride (1 M, 110 mL, 1.00 eq) was added in dropwise. The mixture was then stirred at −78° C. for 1 hour. The reaction mixture was quenched with hydrochloric acid (1 M, 10 mL), filtered through celite. The filtrate was diluted with water (100 mL), then extracted with dichloromethane (100 mL×2). The combined organic layers were dried over anhydrous sodium sulfate, filtered and concentrated under reduced pressure to give a residue. Compound (2R)-2-benzyloxypropanal (15.70 g) was obtained as a colorless oil. LC/MS (ESI) m/z: 181.1 [M+17]⁺; ¹H-NMR (400 MHz, CDCl₃) δ 9.68 (d, J=1.6 Hz, 1H), 7.42-7.33 (m, 5H), 4.68-4.60 (m, 2H), 3.95-3.86 (m, 1H), 1.34 (d, J=6.8 Hz, 3H).

### Step 3: Preparation of (R)-(((1,1-dimethoxypropan-2-yl)oxy)methyl)benzene

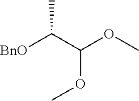

To a solution of (2R)-2-benzyloxypropanal (14.70 g, 89.52 mmol, 1 eq) in trimethoxymethane (71.15 g, 670.46 mmol, 73.5 mL, 7.49 eq) was added 4-methylbenzenesulfonic acid; pyridine (450 mg, 1.79 mmol, 0.02 eq). The mixture was stirred at 25° C. for 2 hours. The reaction mixture was diluted with water (100 mL), then extracted with ethyl acetate (100 mL×2). The combined organic layers were dried over anhydrous sodium sulfate, filtered and concentrated under reduced pressure to give a residue. The residue was purified by silica gel column chromatography (petroleum ether/ethyl acetate=25/1 to 20/1) to give desired product. Compound [(1R)-2,2-dimethoxy-1-methyl-ethoxy]methylbenzene (16.70 g, 79.42 mmol, 89% yield, 100% purity) was obtained as a colorless oil. LC/MS (ESI) m/z: 233.1 [M+23]⁺; ¹H-NMR (400 MHz, CDCl₃) δ 7.39-7.27 (m, 5H), 4.68-4.61 (m, 2H), 4.22 (d, J=5.2 Hz, 1H), 3.62-3.53 (m, 1H), 3.44 (d, J=4.0 Hz, 6H), 1.20 (d, J=6.4 Hz, 3H).

### Step 4: Preparation of (R)-1,1-dimethoxypropan-2-ol

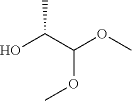

To a solution of [(1R)-2,2-dimethoxy-1-methyl-ethoxy]methylbenzene (9.00 g, 42.80 mmol, 1.00 eq) in methanol (80 mL) was added palladium on activated carbon (500 mg, 5% purity) and palladium hydroxide (500 mg, 5% purity) under nitrogen gas. The suspension was degassed under vacuum and purged with hydrogen gas several times. The mixture was stirred under hydrogen gas (15 psi) at 60° C. for 8 hours. The reaction mixture was filtered through celite and concentrated under reduced pressure to give a residue. Compound (2R)-1,1-dimethoxypropan-2-ol (4.1 g) was obtained as a colorless oil. ¹H-NMR (400 MHz, CDCl₃) δ 4.08 (d, J=6.4 Hz, 1H), 3.81-3.73 (m, 1H), 3.45 (d, J=4.0 Hz, 6H), 1.20 (d, J=6.4 Hz, 3H).

### Step 5: Preparation of tert-butyl (2S)-4-[7-bromo-6-chloro-2-[(1R)-2,2-dimethoxy-1-methyl-ethoxy]-8-fluoro-quinazolin-4-yl]-2-(cyanomethyl)piperazine-1-carboxylate

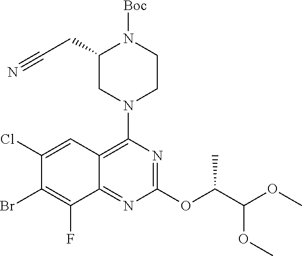 [SMILES: COC(OC)[C@@H](C)Oc1nc(N2CCN(C(=O)OC(C)(C)C)[C@@H](CC#N)C2)c2cc(Cl)c(Br)c(F)c2n1]

To a solution of tert-butyl (2S)-4-(7-bromo-2,6-dichloro-8-fluoro-quinazolin-4-yl)-2-(cyanomethyl)piperazine-1-carboxylate (500 mg, 0.96 mmol, 1.00 eq) and (2R)-1,1-dimethoxypropan-2-ol (231 mg, 1.93 mmol, 2.00 eq) in CH₃CN (5 mL) was added 1,4-diazabicyclo[2.2.2]octane (11 mg, 0.01 mmol, 0.10 eq) and Cs₂CO₃ (408 mg, 1.25 mmol, 1.30 eq), and the reaction mixture was stirred at 45° C. for 2 hours. The solvent was removed under reduced pressure, and the resulting residue was purified by SiO₂ column chromatography (15-25% EtOAc in petroleum ether) to afford tert-butyl (2S)-4-[7-bromo-6-chloro-2-[(1R)-2,2-dimethoxy-1-methyl-ethoxy]-8-fluoro-quinazolin-4-yl]-2-(cyanomethyl)piperazine-1-carboxylate (250 mg, 0.41 mmol, 43% yield) as a pale yellow solid. LC/MS (ESI) m/z: 604.2 [M+H]⁺.

### Step 6: Preparation of tert-butyl (2S)-4-[6-chloro-2-[(1R)-2,2-dimethoxy-1-methyl-ethoxy]-8-fluoro-7-(3-hydroxy-1-naphthyl)quinazolin-4-yl]-2-(cyanomethyl)piperazine-1-carboxylate

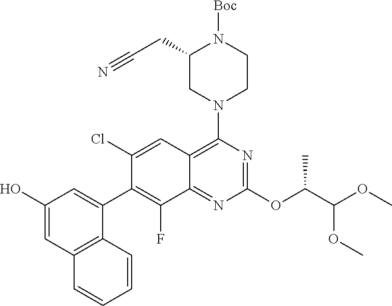 [SMILES: COC(OC)[C@@H](C)Oc1nc(N2CCN(C(=O)OC(C)(C)C)[C@@H](CC#N)C2)c2cc(Cl)c(-c3cc(O)cc4ccccc34)c(F)c2n1]

To a solution of tert-butyl (2S)-4-[7-bromo-6-chloro-2-[(1R)-2,2-dimethoxy-1-methyl-ethoxy]-8-fluoro-quinazolin-4-yl]-2-(cyanomethyl)piperazine-1-carboxylate (250 mg, 0.41 mmol, 1.00 eq) and 4-(4,4,5,5-tetramethyl-1,3,2-dioxaborolan-2-yl)naphthalen-2-ol (129 mg, 0.48 mmol, 1.15 eq) in THF (3.0 mL) were added [2-(2-aminophenyl)phenyl]palladium(1+); dicyclohexyl-[2-(2,4,6-triisopropylphenyl)phenyl]phosphane; methanesulfonate (35 mg, 0.04 mmol, 0.10 eq) and K₃PO₄ (1.5 M, 0.8 mL 3.00 eq), and the reaction mixture was degassed with N₂ gas and stirred at 45° C. for 9 hours. Water (30 mL) was then added, and the resulting mixture was extracted with EtOAc (2×50 mL). The combined organic extracts were washed with brine (30 mL), dried over Na₂SO₄, filtered, and concentrated. The resulting residue was purified by prep-thin layer chromatography (EtOAc: petroleum ether=2:3) to afford tert-butyl (2S)-4-[6-chloro-2-[(1R)-2,2-dimethoxy-1-methyl-ethoxy]-8-fluoro-7-(3-hydroxy-1-naphthyl)quinazolin-4-yl]-2-(cyanomethyl)piperazine-1-carboxylate (180 mg, 0.27 mmol, 65% yield) as a pale red solid. LC/MS (ESI) m/z: 666.4 [M+H]⁺.

### Step 7 Preparation of tert-butyl (2S)-4-[6-chloro-8-fluoro-7-(3-hydroxy-1-naphthyl)-2-[(1R)-1-methyl-2-oxo-ethoxy]quinazolin-4-yl]-2-(cyanomethyl)piperazine-1-carboxylate

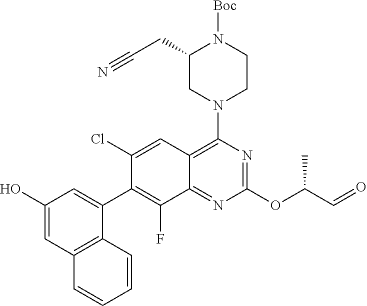 [SMILES: C[C@H](C=O)Oc1nc(N2CCN(C(=O)OC(C)(C)C)[C@@H](CC#N)C2)c2cc(Cl)c(-c3cc(O)cc4ccccc34)c(F)c2n1]

To a solution of tert-butyl (2S)-4-[6-chloro-2-[(1R)-2,2-dimethoxy-1-methyl-ethoxy]-8-fluoro-7-(3-hydroxy-1-naphthyl)quinazolin-4-yl]-2-(cyanomethyl)piperazine-1-carboxylate (130 mg, 0.20 mmol, 1.00 eq) in CH₂Cl₂ (0.5 mL) was added TFA (1.54 g, 13.51 mmol, 1.0 mL, 69.21 eq), and the reaction was stirred at 25° C. for 12 hours. The solvent was removed under reduced pressure and dried in vacuum. The resulting material was taken up in a mixture of THF (1.5 mL) and H₂O (1.5 mL). NaHCO₃ (292 mg, 3.47 mmol, 20.00 eq) was then added in portions followed by di-tert-butyldicarbonate (57 mg, 0.26 mmol, 1.50 eq), and the reaction mixture was stirred at 25° C. for 12 hours. The mixture was diluted with H₂O (20 mL) and extracted with EtOAc (2×20 mL). The combined organic extracts were washed with brine (30 mL), dried over Na₂SO₄, filtered, and concentrated. The resulting residue was purified by prep-thin layer chromatography (CH₂Cl₂:CH₃OH=30:1) to afford tert-butyl (2S)-4-[6-chloro-8-fluoro-7-(3-hydroxy-1-naphthyl)-2-[(1R)-1-methyl-2-oxo-ethoxy]quinazolin-4-yl]-2-(cyanomethyl)piperazine-1-carboxylate (30 mg, 0.05 mmol, 28% yield) as a yellow solid. LC/MS (ESI) m/z: 620.3 [M+H]⁺ and 720.3 [M+Boc+1]⁺.

### Exemplary Synthesis of tert-butyl 4-(6-chloro-8-fluoro-7-(3-hydroxynaphthalen-1-yl)-2-(((R)-1-oxopropan-2-yl)oxy)quinazolin-4-yl)piperazine-1-carboxylate

### Step 5: Preparation of tert-butyl (R)-4-(7-bromo-6-chloro-2-((1,1-dimethoxypropan-2-yl)oxy)-8-fluoroquinazolin-4-yl)piperazine-1-carboxylate

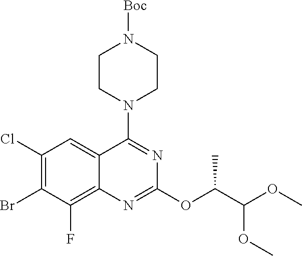 [SMILES: COC(OC)[C@@H](C)Oc1nc(N2CCN(C(=O)OC(C)(C)C)CC2)c2cc(Cl)c(Br)c(F)c2n1]

Tert-butyl 4-(7-bromo-2,6-dichloro-8-fluoro-quinazolin-4-yl)piperazine-1-carboxylate (300 mg, 0.62 mmol, 1 eq), (2R)-1,1-dimethoxypropan-2-ol (150.13 mg, 1.25 mmol, 2 eq), potassium carbonate (259 mg, 1.87 mmol, 3 eq) and 1,4-diazabicyclo[2.2.2]octane (7 mg, 62.48 umol, 0.1 eq) were taken up into a microwave tube in acetonitrile (10 mL). The sealed tube was heated at 100° C. for 2 hours under microwave. The reaction mixture was filtered and the filtrate was concentrated under vacuum to get the residue. The residue was purified by silica gel column chromatography (0-15% ethyl acetate in petroleum ether) to get tert-butyl 4-[7-bromo-6-chloro-2-[(1R)-2,2-dimethoxy-1-methyl-ethoxy]-8-fluoro-quinazolin-4-yl]piperazine-1-carboxylate (706 mg, 1.10 mmol, 22% yield, 87% purity) as a yellow solid. LC/MS (ESI) m/z: 565.1 [M+1]⁺.

### Step 6: Preparation of tert-butyl 4-(6-chloro-2-(((R)-1,1-dimethoxypropan-2-yl)oxy)-8-fluoro-7-(3-hydroxynaphthalen-1-yl)quinazolin-4-yl)piperazine-1-carboxylate

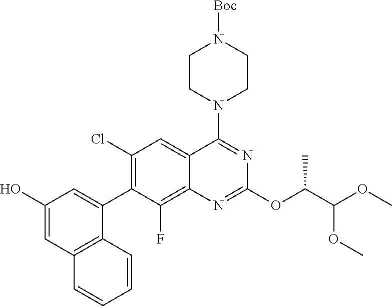 [SMILES: COC(OC)[C@@H](C)Oc1nc(N2CCN(C(=O)OC(C)(C)C)CC2)c2cc(Cl)c(-c3cc(O)cc4ccccc34)c(F)c2n1]

To a solution of tert-butyl 4-[7-bromo-6-chloro-2-[(1R)-2,2-dimethoxy-1-methyl-ethoxy]-8-fluoro-quinazolin-4-yl]piperazine-1-carboxylate (608 mg, 1.08 mmol, 1 eq), 4-(4,4,5,5-tetramethyl-1,3,2-dioxaborolan-2-yl)naphthalen-2-ol (379 mg, 1.40 mmol, 1.3 eq) in tetrahydrofuran (15 mL) was added potassium phosphate (1.5 M, 2.16 mL, 3 eq) and (2-dicyclohexylphosphino-2′,4′,6′-triisopropyl-1,1′-biphenyl)[2-(2′-amino-1,1′-biphenyl)]palladium(ii) methanesulfonate (91 mg, 0.11 mmol, 0.1 eq). The reaction mixture was degassed and charged with nitrogen for 3 times and then heated to 65° C. for 16 hours. Ethyl acetate (30 mL) was added and the mixture was washed with water (30 mL). The organic layer was dried over sodium sulfate and then concentrated under vacuum to get the residue. The residue was purified by flash silica gel chromatography (0-60% ethyl acetate in petroleum ether) to get the crude product (600 mg). This crude product was purified by semi-preparative reverse phase HPLC. The collected fractions were concentrated under vacuum to remove most of the acetonitrile. The pH of the mixture was adjusted to 8 with saturated aqueous sodium bicarbonate and then extracted with ethyl acetate (50 mL×2). The combined organic layer was dried over sodium sulfate and then concentrated under vacuum to get tert-butyl 4-[6-chloro-2-[(1R)-2,2-dimethoxy-1-methyl-ethoxy]-8-fluoro-7-(3-hydroxy-1-naphthyl)quinazolin-4-yl]piperazine-1-carboxylate (400 mg) as a light yellow solid. LC/MS (ESI) m/z: 627.2 [M+1]⁺.

### Step 7: Preparation of (2R)-2-((6-chloro-8-fluoro-7-(3-hydroxynaphthalen-1-yl)-4-(piperazin-1-yl)quinazolin-2-yl)oxy)propanal

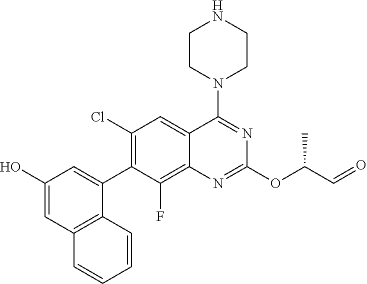 [SMILES: C[C@H](C=O)Oc1nc(N2CCNCC2)c2cc(Cl)c(-c3cc(O)cc4ccccc34)c(F)c2n1]

To a solution of tert-butyl 4-[6-chloro-2-[(1R)-2,2-dimethoxy-1-methyl-ethoxy]-8-fluoro-7-(3-hydroxy-1-naphthyl)quinazolin-4-yl]piperazine-1-carboxylate (400 mg, 0.64 mmol, 1 eq) in dioxane (20 mL) was added hydrochloric acid (12 M, 2.00 mL, 37.63 eq). The reaction mixture was stirred at 25° C. for 1 hour. The reaction mixture was concentrated under vacuum to get (2R)-2-[6-chloro-8-fluoro-7-(3-hydroxy-1-naphthyl)-4-piperazin-1-yl-quinazolin-2-yl]oxypropanal (330 mg, hydrochloride) as a light yellow gum. LC/MS (ESI) m/z: 481.1 [M+1]⁺.

### Step 8: Preparation of tert-butyl 4-(6-chloro-8-fluoro-7-(3-hydroxynaphthalen-1-yl)-2-(((R)-1-oxopropan-2-yl)oxy)quinazolin-4-yl)piperazine-1-carboxylate

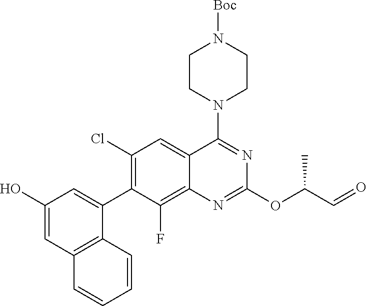 [SMILES: C[C@H](C=O)Oc1nc(N2CCN(C(=O)OC(C)(C)C)CC2)c2cc(Cl)c(-c3cc(O)cc4ccccc34)c(F)c2n1]

A mixture of (2R)-2-[6-chloro-8-fluoro-7-(3-hydroxy-1-naphthyl)-4-piperazin-1-yl-quinazolin-2-yl]oxypropanal (330 mg, 0.64 mmol, 1 eq, hydrochloride) and di-tert-butyl dicarbonate (278.41 mg, 1.28 mmol, 2 eq) in tetrahydrofuran (20 mL) was cooled to 0° C. Then saturated aqueous sodium sulfate (322 mg, 3.83 mmol, 6 mL, 6 eq) was added. The reaction mixture was stirred at 25° C. for 2 hours. Ethyl acetate (30 mL) and water (20 mL) were added and the mixture was separated. The organic layer was dried over sodium sulfate and then concentrated under vacuum to get the residue. The residue was purified by silica gel column chromatography with dichloromethane (50 mL) then ethyl acetate (40 mL) to get tert-butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (380 mg) as a light yellow solid. LC/MS (ESI) m/z: 581.2 [M+1]⁺; ¹H-NMR (400 MHz, CDCl₃) δ 9.71 (dd, J=1.8, 3.4 Hz, 1H), 7.80 (d, J=1.1 Hz, 1H), 7.76 (d, J=8.3 Hz, 1H), 7.44 (dt, J=1.5, 7.3 Hz, 1H), 7.33-7.27 (m, 2H), 7.27-7.22 (m, 1H), 7.10 (d, J=2.4 Hz, 1H), 6.32-5.88 (m, 1H), 5.30-5.22 (m, 1H), 3.99-3.77 (m, 4H), 3.74-3.61 (m, 4H), 1.59-1.54 (m, 3H), 1.52 (s, 9H).

### Exemplary Synthesis of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide Hydrochloride

### Step 1: Preparation of tert-butyl (S)-(1-(4-bromophenyl)ethyl)carbamate

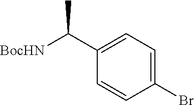 [SMILES: C[C@H](NC(=O)OC(C)(C)C)c1ccc(Br)cc1]

Into a 250-mL round-bottom flask, was placed (1S)-1-(4-bromophenyl)ethan-1-amine (10.0 g, 49.98 mmol, 1.00 equiv) in dichloromethane (100 mL), triethylamine (10.0 g, 99.01 mmol, 2.00 equiv), di-tert-butyl dicarbonate (13.0 g, 59.63 mmol, 1.20 equiv). The resulting solution was stirred for 2 hours at room temperature. The resulting mixture was concentrated under vacuum. The residue was applied onto a silica gel column with ethyl acetate/petroleum ether (1:10). This resulted in 15.0 g of tert-butyl N-[(1S)-1-(4-bromophenyl)ethyl]carbamate as a white solid.

### Step 2: Preparation of tert-butyl (S)-(1-(4-(4-methylthiazol-5-yl)phenyl)ethyl)carbamate

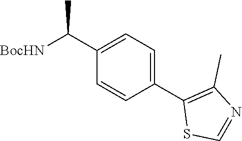 [SMILES: Cc1ncsc1-c1ccc([C@H](C)NC(=O)OC(C)(C)C)cc1]

Into a 250-mL round-bottom flask purged and maintained with an inert atmosphere of nitrogen, was placed a solution of tert-butyl N-[(1S)-1-(4-bromophenyl)ethyl]carbamate (15.0 g, 49.97 mmol, 1.00 equiv) in N,N-Dimethylacetamide (100 mL), 4-methyl-1,3-thiazole (9.9 g, 99.84 mmol, 2.00 equiv), potassium acetate (9.8 g, 99.86 mmol, 2.00 equiv), palladium(II) acetate (112.5 mg, 0.50 mmol, 0.01 equiv). The resulting solution was stirred for 2 hours at 120° C. The reaction mixture was quenched by the addition of water (500 mL). The resulting solution was extracted with ethyl acetate (200 mL×3) and the organic layers combined and concentrated under vacuum. The residue was applied onto a silica gel column with ethyl acetate/petroleum ether (1:5). This resulted in 7.5 g (47%) of tert-butyl N-[(1S)-1-[4-(4-methyl-1,3-thiazol-5-yl)phenyl]ethyl]carbamate as a white solid. LC/MS (ESI) m/z: 319.13 [M+Na]⁺.

### Step 3: Preparation of (S)-1-(4-(4-methylthiazol-5-yl)phenyl)ethan-1-amine Hydrochloride

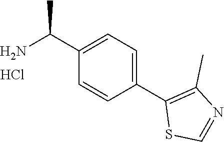

Into a 100-mL round-bottom flask, was placed a solution of tert-butyl N-[(1S)-1-[4-(4-methyl-1,3-thiazol-5-yl)phenyl]ethyl]carbamate (7.5 g, 23.55 mmol, 1.00 equiv) in methanol (20 mL), hydrogen chloride (gas) was bubbled in at room temperature. The resulting solution was stirred for 2 hours at room temperature. The resulting mixture was concentrated under vacuum. This resulted in 4.4 g (86%) of (1S)-1-[4-(4-methyl-1,3-thiazol-5-yl)phenyl]ethan-1-amine as a white solid.

### Step 4: Preparation of tert-butyl (2S,4R)-4-hydroxy-2-(((S)-1-(4-(4-methylthiazol-5-yl)phenyl)ethyl)carbamoyl)pyrrolidine-1-carboxylate

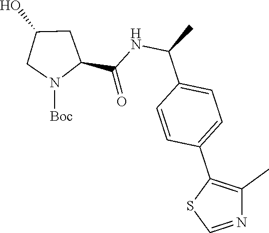 [SMILES: Cc1ncsc1-c1ccc([C@H](C)NC(=O)[C@@H]2C[C@@H](O)CN2C(=O)OC(C)(C)C)cc1]

Into a 100-mL round-bottom flask, was placed (2S,4R)-1-[(tert-butoxy)carbonyl]-4-hydroxypyrrolidine-2-carboxylic acid (4.7 g, 20.32 mmol, 1.00 equiv) in N,N-dimethylformamide (20 mL), N-ethyl-N-isopropylpropan-2-amine (7.8 g, 60.35 mmol, 3.00 equiv), o-(7-Azabenzotriazol-1-yl)-N,N,N′,N′-tetramethyluronium hexafluorophosphate (11.5 g, 30.26 mmol, 1.50 equiv), (1S)-1-[4-(4-methyl-1,3-thiazol-5-yl)phenyl]ethan-1-amine (4.4 g, 20.15 mmol, 1.00 equiv). The resulting solution was stirred for 12 hours at room temperature. The reaction mixture was quenched by the addition of water (20 mL). The resulting solution was extracted with ethyl acetate (100 mL×3) and the organic layers combined and dried in an oven under reduced pressure, concentrated under vacuum. The residue was applied onto a silica gel column with ethyl acetate/petroleum ether (1:1). This resulted in 5.0 g (57%) of tert-butyl (2S ,4R)-4-hydroxy-2-[[(1S)-1-[4-(4-methyl-1,3-thiazol-5-yl)phenyl]ethyl]carbamoyl]pyrrolidine-1-carboxylate as a yellow solid. LC/MS (ESI) m/z: 432.15 [M+1]⁺.

### Step 5: Preparation of (2S,4R)-4-hydroxy-N—((S)-1-(4-(4-methylthiazol-5-yl)phenyl)ethyl)pyrrolidine-2-carboxamide Hydrochloride

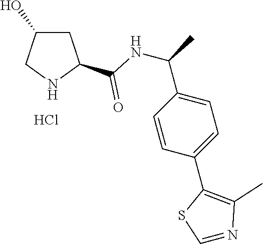 [SMILES: Cc1ncsc1-c1ccc([C@H](C)NC(=O)[C@@H]2C[C@@H](O)CN2)cc1 ; Cl]

Into a 500-mL round-bottom flask, was placed a solution of tert-butyl (2S,4R)-4-hydroxy-2-[[(1S)-1-[4-(4-methyl-1,3-thiazol-5-yl)phenyl]ethyl]carbamoyl]pyrrolidine-1-carboxylate (5.0 g, 11.59 mmol, 1.00 equiv) in methanol (200 mL), then hydrogen chloride (gas) was bubbled into the reaction mixture for 2 hours at room temperature. The resulting mixture was concentrated under vacuum. This resulted in 3.2 g (83%) of (2S,4R)-4-hydroxy-N-[(1S)-1-[4-(4-methyl-1,3-thiazol-5-yl)phenyl]ethyl]pyrrolidine-2-carboxamide as a red solid.

### Step 6: Preparation of tert-butyl ((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamate

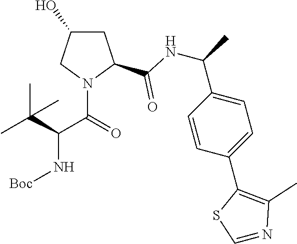 [SMILES: Cc1ncsc1-c1ccc([C@H](C)NC(=O)[C@@H]2C[C@@H](O)CN2C(=O)[C@@H](NC(=O)OC(C)(C)C)C(C)(C)C)cc1]

Into a 25-mL round-bottom flask, was placed (2S)-2-[(tert-butoxy)carbonyl]amino-3,3-dimethylbutanoic acid (2.0 g, 8.65 mmol, 0.99 equiv) in N,N-dimethylformamide (30 mL). N-ethyl-N-isopropylpropan-2-amine (3.4 g, 3.00 equiv), o-(7-Azabenzotriazol 1)-N,N,N′,N′-tetramethyluronium hexafluorophosphate (5.0 g, 1.50 equiv), (2S,4R)-4-hydroxy-N-[(1S)-1-[4-(4-methyl-1,3-thiazol-5-yl)phenyl]ethyl]pyrrolidine-2-carboxamide hydrochloride (3.2 g, 8.70 mmol, 1.00 equiv). The resulting solution was stirred for 12 hours at room temperature. The resulting solution was extracted with ethyl acetate (60 mL×3) and washed with water (100 mL×2). The organic layers combined and dried, concentrated under vacuum. The residue was applied onto a silica gel column with ethyl acetate/petroleum ether (1:3). This resulted in 4.0 g (84%) of tert-butyl N-[(2S)-1-[(2S ,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamate as a yellow solid. LC/MS (ESI) m/z: 545.30 [M+1]⁺.

### Step 7: Preparation of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide Hydrochloride

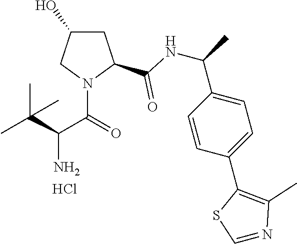 [SMILES: Cc1ncsc1-c1ccc([C@H](C)NC(=O)[C@@H]2C[C@@H](O)CN2C(=O)[C@@H](N)C(C)(C)C)cc1 ; Cl]

Into a 100-mL round-bottom flask, was placed a solution of tert-butyl N-[(2S)-1-[(2S,4R)-4-hydroxy-2-[[(1S)-1-[4-(4-methyl-1,3-thiazol-5-yl)phenyl]ethyl]carbamoyl]pyrrolidin-1-yl]-3,3-dimethyl-1-oxobutan-2-yl]carbamate (4.0 g, 7.34 mmol, 1.00 equiv) in methanol (30 mL), then hydrogen chloride (gas) was bubbled into the reaction mixture for 2 hours at room temperature. The resulting mixture was concentrated under vacuum. This resulted in 3.5 g of (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride as a yellow solid. LC/MS (ESI) m/z: 445.05 [M+1]⁺; ¹H-NMR (400 MHz, DMSO-d₆) δ 8.99 (s, 1H), 8.57-8.55 (d, J=7.8 Hz, 1H), 8.01 (b, 3H), 7.46-7.43 (d, J=8.4 Hz, 2H), 7.39-7.37 (d, J=8.4 Hz, 2H), 4.98-4.90 (m, 1H), 4.57-4.51 (m, 1H), 4.34 (b, 1H), 3.94-3.92 (m, 1H), 3.69-3.66 (m, 1H), 3.53-3.49 (m, 1H), 2.52 (s, 3H), 2.10-2.07 (m, 1H), 1.83-1.81 (m, 1H), 1.40-1.30 (m, 3H), 1.03 (s, 9H).

### Exemplary Synthesis of (2S,4R)-4-hydroxy-N-(2-hydroxy-4-(4-methylthiazol-5-yl)benzyl)-1-(3-methyl-2-(3-methylisoxazol-5-yl)butanoyl)pyrrolidine-2-carboxamide

### Step 1: Preparation of 2-(3-methylisoxazol-5-yl)acetic Acid

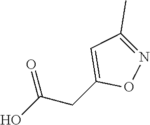

To a solution of 3,5-dimethylisoxazole (15 g, 154.46 mmol, 15 mL, 1 eq) in tetrahydrofuran (150 mL) was added n-butyllithium (2.5 M, 77 mL, 1.25 eq) dropwise at −78° C. under nitrogen, the mixture was stirred at −55° C. for 30 minutes, and then carbon dioxide was bubbled into the mixture for 30 minutes, the mixture was stirred at 25° C. for 1 hour. The mixture was quenched by saturated ammonium chloride solution (50 mL) the mixture was extracted with ethyl acetate (50 mL). The aqueous phase was adjusted with aqueous hydrochloric acid solution (2 M) until pH=2, the mixture was extracted with ethyl acetate (50 mL, three times), the organic phase was dried by anhydrous sodium sulfate, filtered and the filtrate was concentrated to give 2-(3-methylisoxazol-5-yl)acetic acid (10 g, 70.86 mmol, 46% yield) as a brown solid. ¹H-NMR (400 MHz, DMSO-d₆) δ 12.74 (br s, 1H), 6.24 (s, 1H), 3.83 (s, 2H), 2.20 (s, 3H).

### Step 2: Preparation of methyl 2-(3-methylisoxazol-5-yl)acetate

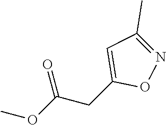

To a solution of 2-(3-methylisoxazol-5-yl)acetic acid (10 g, 70.86 mmol, 1 eq) in methanol (100 mL) was added thionyl chloride (12.65 g, 106.29 mmol, 7.71 mL, 1.5 eq) at 0° C., and the mixture was stirred at 50° C. for 4 hours. The mixture was concentrated to give crude product. This crude was diluted with ethyl acetate (200 mL) and washed by water (200 mL), and then saturated sodium bicarbonate aqueous solution (50 mL) and then brine (50 mL), the organic phase was dried by anhydrous, filtered and the filtrate was condensed to give methyl 2-(3-methylisoxazol-5-yl)acetate (10 g, 64.45 mmol, 91% yield) as a brown oil. ¹H-NMR (400 MHz, CDCl₃) δ 6.11 (s, 1H), 3.80 (s, 2H), 3.76 (s, 3H), 2.30 (s, 3H).

### Step 3: Preparation of methyl 3-methyl-2-(3-methylisoxazol-5-yl)butanoate

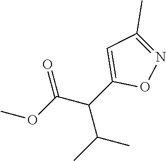

To a solution of methyl 2-(3-methylisoxazol-5-yl)acetate (10 g, 64.45 mmol, 1 eq) in tetrahydrofuran (100 mL) was added sodium hydride (3.87 g, 96.68 mmol, 60% purity, 1.5 eq) at 0° C. and then 2-iodopropane (13.15 g, 77.34 mmol, 7.74 mL, 1.2 eq) was added at 0° C., the mixture was stirred at 25° C. for 2 hours. Additional 2-iodopropane (2.55 g, 15.00 mmol, 1.5 mL) was added and the mixture was stirred at 25° C. for 10 hours. The mixture was quenched by aqueous hydrochloric acid solution (1 M, 300 mL) and the mixture was extracted with ethyl acetate (200 mL, three times), the organic phase was dried by anhydrous sodium sulfate, filtered and the filtrate was concentrated to give methyl 3-methyl-2-(3-methylisoxazol-5-yl)butanoate (13 g) as a brown oil.

### Step 4: Preparation of 3-methyl-2-(3-methylisoxazol-5-yl)butanoic Acid

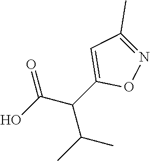

To a solution of methyl 3-methyl-2-(3-methylisoxazol-5-yl)butanoate (12.7 g, 64.39 mmol, 1 eq) in methanol (90 mL) and water (60 mL) was added sodium hydroxide (12.88 g, 321.96 mmol, 5 eq), the mixture was stirred at 25° C. for 2 hours. The mixture was concentrated to removed methanol, and then the residue was diluted with water (200 mL) and extracted with ethyl acetate (200 mL), the aqueous phase was adjusted by aqueous hydrochloric acid solution (2 M) until pH=3, and then the mixture was extracted with dichloromethane (200 mL, three times), the organic phase was dried by anhydrous sodium sulfate, filtered and the filtrate was concentrated to give crude product as a brown oil, this crude was purified by flash prep-HPLC, the fraction of acetonitrile was removed and the residue was extracted with dichloromethane (300 mL×5), the organic phase was dried by anhydrous sodium sulfate, filtered and the filtrate was concentrated to give product 3-methyl-2-(3-methylisoxazol-5-yl)butanoic acid (7.5 g, 40.94 mmol, 63% yield) as white solid. ¹H-NMR (400 MHz, DMSO-d₆) δ 6.26 (s, 1H), 3.58 (d, J=8.7 Hz, 1H), 2.33-2.23 (m, 1H), 2.21 (s, 3H), 0.95 (d, J=6.7 Hz, 3H), 0.82 (d, J=6.8 Hz, 3H).

### Step 5: Preparation of 2-hydroxy-4-(4-methylthiazol-5-yl)benzonitrile

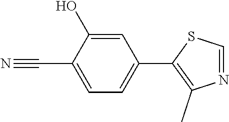

To a solution of 4-bromo-2-hydroxy-benzonitrile (15 g, 75.75 mmol, 1 eq) and 4-methylthiazole (20.28 g, 204.53 mmol, 19 mL, 2.7 eq) in N-methyl pyrrolidone (150 mL) was added potassium acetate (22.30 g, 227.25 mmol, 3 eq) and palladium acetate (1.70 g, 7.58 mmol, 0.1 eq)), the mixture stirred at 110° C. under nitrogen for 6 hours. The mixture was quenched with water (500 mL), the aqueous phase was extracted with ethyl acetate (300 mL×3). The combined organic phase was washed with brine (200 mL, twice), dried with anhydrous sodium sulfate, filtered and concentrated under vacuum and then methyl tertiary butyl ether (500 mL) was added to the mixture and the organic phase was washed with water (100 mL) and brine (100 mL, twice). The residue was purified by silica gel column chromatography (petroleum ether/ethyl acetate=3/1 to 1/1). Compound 2-hydroxy-4-(4-methylthiazol-5-yl)benzonitrile (11 g, 50.87 mmol, 67% yield) was obtained as a yellow solid.

### Step 6: Preparation of 2-(aminomethyl)-5-(4-methylthiazol-5-yl)phenol

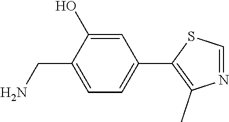

To a solution of 2-hydroxy-4-(4-methylthiazol-5-yl)benzonitrile (11 g, 50.87 mmol, 1 eq) in tetrahydrofuran (150 mL) was added lithium aluminum hydride (7.72 g, 203.46 mmol, 4 eq) at 0° C., the mixture was stirred at 50° C. for 3 hours. The mixture was quenched by water (8 mL) at 0° C., and then 15% sodium hydroxide aqueous solution (8 mL) and then water (8 mL), anhydrous sodium sulfate (30 g) was added, the mixture was stirred at 25° C. for 30 minutes, filtered and the solid was added dichloromethane/methanol (4/1, 50 mL), the mixture was stirred at 25° C. for 1 hours, filtered and the filtrate combined was concentrated to give 2-(aminomethyl)-5-(4-methylthiazol-5-yl)phenol (7 g, 31.78 mmol, 62% yield) as a brown solid. ¹H-NMR (400 MHz, DMSO-d₆) δ 8.82 (s, 1H), 6.90 (d, J=7.5 Hz, 1H), 6.52 (d, J=1.6 Hz, 1H), 6.25 (dd, J=1.7, 7.5 Hz, 1H), 3.59 (s, 2H), 2.41 (s, 3H).

### Step 7: Preparation of tert-butyl (2S,4R)-4-hydroxy-2-((2-hydroxy-4-(4-methylthiazol-5-yl)benzyl)carbamoyl)pyrrolidine-1-carboxylate

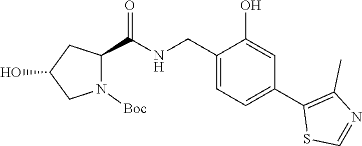 [SMILES: Cc1ncsc1-c1ccc(CNC(=O)[C@@H]2C[C@@H](O)CN2C(=O)OC(C)(C)C)c(O)c1]

To a solution of 2-(aminomethyl)-5-(4-methylthiazol-5-yl)phenol (7 g, 31.78 mmol, 1 eq) and (2S,4R)-1-tert-butoxycarbonyl-4-hydroxy-pyrrolidine-2-carboxylic acid (7.35 g, 31.78 mmol, 1 eq) in dimethylformamide (70 mL) was added diisopropylethylamine (12.32 g, 95.33 mmol, 16.60 mL, 3 eq) and then HATU (13.29 g, 34.95 mmol, 1.1 eq), the mixture was stirred at 25° C. for 2 hours. Additional (2S,4R)-1-tert-butoxycarbonyl-4-hydroxy-pyrrolidine-2-carboxylic acid (7.35 g, 31.78 mmol, 1 eq) and HATU (12.08 g, 31.78 mmol, 1 eq) was added, the mixture was stirred at 25° C. for 5 hours. The mixture was diluted with water (300 mL) and extracted with ethyl acetate (300 mL, twice), the organic phase was dried by anhydrous sodium sulfate, filtered and the filtrate was concentrated to give crude product as a brown oil, this crude was dissolved in tetrahydrofuran/water (2/1, 150 mL) and lithium hydroxide (3 g) was added, the mixture was stirred at 25° C. for 1 hour. The mixture was diluted with water (300 mL) and adjusted with aqueous hydrochloric acid solution (0.5 M) until pH=7, the mixture was extracted with ethyl acetate (300 mL, twice), the organic phase was dried by anhydrous sodium sulfate, filtered and filtrate was concentrated to give crude product, this crude product was purified by silica gel chromatography (2-10% methanol in dichloromethane) to give tert-butyl (2S,4R)-4-hydroxy-2-[[2-hydroxy-4-(4-methylthiazol-5-yl)phenyl]methylcarbamoyl]pyrrolidine-1-carboxylate (6.9 g, 15.92 mmol, 50% yield) as a yellow oil. LC/MS (ESI) m/z: 434.1 [M+1]⁺.

### Step 8: Preparation of (2S,4R)-4-hydroxy-N-(2-hydroxy-4-(4-methylthiazol-5-yl)benzyl)pyrrolidine-2-carboxamide

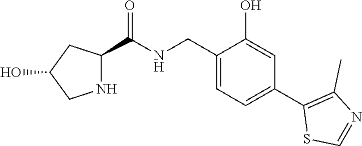 [SMILES: Cc1ncsc1-c1ccc(CNC(=O)[C@@H]2C[C@@H](O)CN2)c(O)c1]

To a solution of tert-butyl (2S,4R)-4-hydroxy-2-[[2-hydroxy-4-(4-methylthiazol-5-yl)phenyl]methylcarbamoyl]pyrrolidine-1-carboxylate (6.9 g, 15.92 mmol, 1 eq) in methanol (30 mL) was added hydrochloric/dioxane (4 M, 30 mL, 7.54 eq), the mixture was stirred at 25° C. for 20 minutes. The mixture was concentrated to give product as a yellow solid, this crude product was triturated by ethyl acetate and petroleum ether (1:1, 20 mL), the mixture was filtered and the solid was dried by rotary evaporator to give product (2S,4R)-4-hydroxy-N-[[2-hydroxy-4-(4-methylthiazol-5-yl)phenyl]methyl]pyrrolidine-2-carboxamide (4.83 g, 13.06 mmol, 82% yield, hydrochloric acid) as a yellow solid. ¹H-NMR (400 MHz, DMSO-d₆) δ 10.03 (br s, 1H), 9.11-8.95 (m, 2H), 8.66 (br s, 1H), 7.20 (d, J=7.9 Hz, 1H), 7.04 (d, J=1.3 Hz, 1H), 6.90 (dd, J=1.7, 7.8 Hz, 1H), 4.44 (br s, 1H), 4.40-4.26 (m, 3H), 3.41-3.27 (m, 1H), 3.13-3.02 (m, 1H), 2.46 (s, 3H), 2.33 (br dd, J=7.5, 12.7 Hz, 1H), 1.96-1.85 (m, 1H), 1.33-1.24 (m, 1H).

### Step 9: Preparation of (2S,4R)-4-hydroxy-N-(2-hydroxy-4-(4-methylthiazol-5-yl)benzyl)-1-(3-methyl-2-(3-methylisoxazol-5-yl)butanoyl)pyrrolidine-2-carboxamide

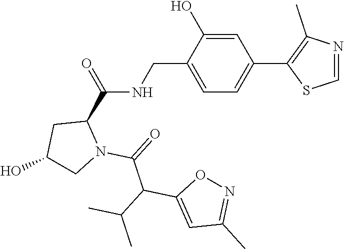 [SMILES: Cc1cc(C(C(=O)N2C[C@H](O)C[C@H]2C(=O)NCc2ccc(-c3scnc3C)cc2O)C(C)C)on1]

To a solution of (2S,4R)-4-hydroxy-N-[[2-hydroxy-4-(4-methylthiazol-5-yl)phenyl]methyl]pyrrolidine-2-carboxamide (4.83 g, 13.06 mmol, 1 eq, hydrochloride) in dimethylformamide (60 mL) was added diisopropylethylamine (5.06 g, 39.18 mmol, 6.82 mL, 3 eq), and then 3-methyl-2-(3-methylisoxazol-5-yl)butanoic acid (2.39 g, 13.06 mmol, 1 eq) and HATU (5.46 g, 14.36 mmol, 1.1 eq) was added, the mixture was stirred at 25° C. for 2 hours. The mixture was diluted with water (200 mL) and extracted with ethyl acetate (300 mL, twice), the organic phase was dried by anhydrous sodium sulfate, filtered and the filtrate was concentrated to give crude product. This crude product was purified by prep-HPLC, the fraction of acetonitrile was removed, and the residue was extracted with dichloromethane (300 mL×5), the organic phase was dried by anhydrous sodium sulfate, filtered and the filtrate was concentrated to give product (2S,4R)-4-hydroxy-N-[[2-hydroxy-4-(4-methylthiazol-5-yl)phenyl]methyl]-1-[3-methyl-2-(3-methylisoxazol-5-yl)butanoyl]pyrrolidine-2-carboxamide (4.0 g, 8.02 mmol, 61% yield) as a white solid. ¹H-NMR (400 MHz, CD₃OD) δ 8.85 (s, 1H), 7.39-7.23 (m, 1H), 6.98-6.86 (m, 2H), 6.31-6.06 (m, 1H), 4.65-4.28 (m, 4H), 3.94-3.48 (m, 3H), 2.52-2.45 (m, 3H), 2.42-2.31 (m, 1H), 2.26-2.15 (m, 4H), 2.13-2.03 (m, 1H), 1.08-1.01 (m, 3H), 0.92-0.81 (m, 3H).

### Exemplary Synthesis of tert-butyl (S)-4-(4-((benzyloxy)carbonyl)-3-(cyanomethyl)piperazin-1-yl)-2-chloro-5,8-dihydropyrido[3,4-d]pyrimidine-7(6H)-carboxylate

### Step 1: Preparation of benzyl (S)-2-(cyanomethyl)piperazine-1-carboxylate

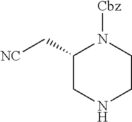 [SMILES: N#CC[C@H]1CNCCN1C(=O)OCc1ccccc1]

To a solution of O1-benzyl-((4-tert-butyl (2S)-2-(cyanomethyl)piperazine-1,4-dicarboxylate (900 mg, 2.50 mmol, 1.00 eq) in dichloromethane (10 mL) was added trifluoroacetic acid (3.08 g, 27.00 mmol, 10.79 eq) slowly. The solution was stirred at 10° C. for 2 hours. The solution was concentrated under reduced pressure to give benzyl (2S)-2-(cyanomethyl)piperazine-1-carboxylate (911 mg, 2.44 mmol, 97% yield, trifluoroacetic acid salt) as yellow liquid.

### Step 2: Preparation of tert-butyl (S)-4-(4-((benzyloxy)carbonyl)-3-(cyanomethyl)piperazin-1-yl)-2-chloro-5,8-dihydropyrido[3,4-d]pyrimidine-7(6H)-carboxylate

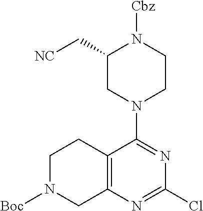 [SMILES: CC(C)(C)OC(=O)N1CCc2c(nc(Cl)nc2N2CCN(C(=O)OCc3ccccc3)[C@@H](CC#N)C2)C1]

To a solution of benzyl (2S)-2-(cyanomethyl)piperazine-1-carboxylate (646 mg, 2.49 mmol, 1.00 eq, trifluoroacetic acid salt) and Diisopropylethylamine (1.29 g, 9.96 mmol, 4.00 eq) in dimethylsulfoxide (20 mL) was added tert-butyl 2,4-dichloro-5,6-dihydropyrido[3,4-d]pyrimidine-7(8H)-carboxylate (758 mg, 2.49 mmol, 1.00 eq) in one portion. The resulted solution was stirred at 50° C. for 9 hours. The reaction solution was diluted with ethyl acetate (200 mL) and water (100 mL). The organic layer was separated and collected, washed with water (50 mL×2) and brine (50 mL), dried over anhydrous sodium sulfate, concentrated under reduced pressure to give a yellow liquid. The yellow liquid was purified by column chromatography (silicon dioxide, Petroleum ether/Ethyl acetate=10/1 to 1/1) to obtained tert-butyl 4-[(3S)-4-benzyloxycarbonyl-3-(cyanomethyl) piperazin-1-yl]-2-chloro-6,8-dihydro-5H-pyrido[3,4-d]pyrimidine-7-carboxylate (1.10 g, 2.09 mmol, 84% yield) as yellow liquid. LC/MS (ESI) m/z: 527.1 [M+1]⁺; ¹H-NMR (400 MHz, CDCl₃) δ 7.46-7.32 (m, 5H), 5.26-5.14 (m, 2H), 4.67 (d, J=17.6 Hz, 2H), 4.51-4.42 (m, 1H), 4.21-4.05 (m, 2H), 3.93-3.75 (m, 2H), 3.40 (d, J=10.8 Hz, 2H), 3.12 (dt, J=3.2, 12.4 Hz, 1H), 2.97-2.51 (m, 3H), 1.61 (s, 2H), 1.50 (s, 9H).

### Exemplary Synthesis of (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide

### Step 1: Preparation of tert-butyl 4-(2-(2-ethoxy-2-oxoethoxy)ethoxy)piperidine-1-carboxylate

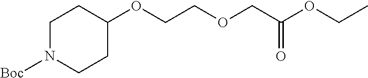 [SMILES: CCOC(=O)COCCOC1CCN(C(=O)OC(C)(C)C)CC1]

To a solution of tert-butyl 4-(2-hydroxyethoxy)piperidine-1-carboxylate (2 g, 8.15 mmol, 1 eq) in dichloromethane (20 mL) was added diacetoxyrhodium (90 mg, 0.41 mmol, 0.05 eq). ethyl 2-diazoacetate (2.79 g, 24.46 mmol, 3 eq) was added at 0° C., the mixture was stirred at 25° C. for 12 hours. The reaction mixture was concentrated under vacuum. The crude product was purified by flash silica gel chromatography (Petroleum ether: Ethyl acetate=9:1 to 3:1). Compound tert-butyl 4-[2-(2-ethoxy-2-oxo-ethoxy)ethoxy]piperidine-1-carboxylate (1.7 g, 5.13 mmol, 63% yield) as a colorless oil was obtained. ¹H-NMR (400 MHz, CDCl₃) δ 4.22 (q, J=7.1 Hz, 2H), 4.16 (s, 2H), 3.83-3.71 (m, 4H), 3.70-3.63 (m, 2H), 3.50 (tt, J=3.9, 8.2 Hz, 1H), 3.07 (ddd, J=3.4, 9.5, 13.3 Hz, 2H), 1.89-1.79 (m, 2H), 1.58-1.48 (m, 2H), 1.46 (s, 9H), 1.32-1.27 (m, 3H).

### Step 2: Preparation of 2-(2-((1-(tert-butoxycarbonyl)piperidin-4-yl)oxy)ethoxy)acetic Acid

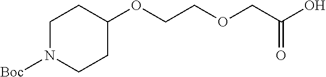 [SMILES: CC(C)(C)OC(=O)N1CCC(OCCOCC(=O)O)CC1]

To a solution of tert-butyl 4-[2-(2-ethoxy-2-oxo-ethoxy)ethoxy]piperidine-1-carboxylate (1.6 g, 4.83 mmol, 1 eq) in methanol (3 mL) and tetrahydrofuran (3 mL) and water (3 mL) was added lithium hydroxide monohydrate (405 mg, 9.66 mmol, 2 eq). The mixture was stirred at 25° C. for 1 hour. Water 10 mL was added. The mixture was adjusted pH to 3-4 by 1M hydrochloric acid, and then the aqueous phase was extracted with dichloromethane and methanol (10:1, 30 mL x 3). The combined organic phase was dried with anhydrous sodium sulfate, filtered and concentrated in vacuum. Compound 2-[2-[(1-tert-butoxycarbonyl-4-piperidyl)oxy]ethoxy]acetic acid (1.2 g, 3.96 mmol, 82% yield) as a yellow solid was obtained.

### Step 3: Preparation of tert-butyl 4-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)piperidine-1-carboxylate

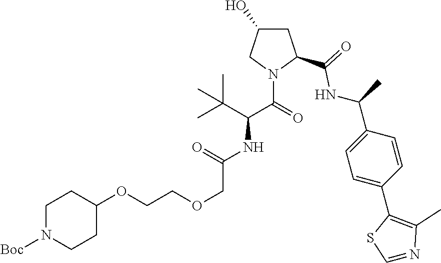 [SMILES: Cc1ncsc1-c1ccc([C@H](C)NC(=O)[C@@H]2C[C@@H](O)CN2C(=O)[C@@H](NC(=O)COCCOC2CCN(C(=O)OC(C)(C)C)CC2)C(C)(C)C)cc1]

To a solution of 2-[2-[(1-tert-butoxycarbonyl-4-piperidyl)oxy]ethoxy]acetic acid (410 mg, 1.35 mmol, 1 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (600 mg, 1.35 mmol, 1 eq) in N,N-dimethylformamide (10 mL) was added hydroxybenzotriazole (274 mg, 2.03 mmol, 1.50 eq) and 1-(3-dimethylaminopropyl)-3-ethylcarbodiimide hydrochloride (389 mg, 2.03 mmol, 1.50 eq) and N,N-diisopropylethylamine (593 mg, 4.59 mmol, 0.8 mL, 3.40 eq). The mixture was stirred at 25° C. for 12 hours. Water (50 mL) was added, the aqueous phase was extracted with ethyl acetate (40 mL×3). The combined organic phase was washed with brine (20 mL), dried with anhydrous sodium sulfate, filtered and concentrated in vacuum. The residue was purified by silica gel chromatography (Dichloromethane:Methanol=1:0 to 20:1). Compound tert-butyl 4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]piperidine-1-carboxylate (690 mg, 0.95 mmol, 70% yield) was obtained as a yellow oil. LC/MS (ESI) m/z: 730.4 [M+1]⁺.

### Step 4: Preparation of (2S,4R)-1-((S)-3,3-dimethyl-2-(2-(2-(piperidin-4-yloxy)ethoxy)acetamido)butanoyl)-4-hydroxy-N—((S)-1-(4-(4-methylthiazol-5-yl)phenyl)ethyl)pyrrolidine-2-carboxamide

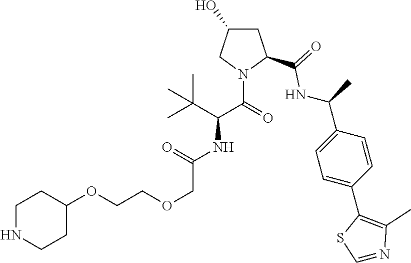 [SMILES: Cc1ncsc1-c1ccc([C@H](C)NC(=O)[C@@H]2C[C@@H](O)CN2C(=O)[C@@H](NC(=O)COCCOC2CCNCC2)C(C)(C)C)cc1]

A mixture of tert-butyl 4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]piperidine-1-carboxylate (690 mg, 0.95 mmol, 1.00 eq) in hydrochloric acid/dioxane (4.0 M, 15 mL, 63.47 eq) was stirred at 20° C. for 1.0 hour. The solvent was removed under reduced pressure. The residue was diluted with methanol (10 mL) and acetonitrile (30 mL), the solvent was removed again and dried in vacuum. A suspension of (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (470 mg, 705.42 umol, 1 eq, hydrochloride) and potassium carbonate (975 mg, 7.05 mmol, 10.00 eq) in a mixture of dichloromethane (8 mL) and acetonitrile (16 mL) was stirred at 25° C. for 1.5 hours. The suspension was filtered through a celite pad and washed with dichloromethane (15 mL), the filtrate was concentrated and dried in vacuum. Compound (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (295 mg, 0.47 mmol, 66% yield) was obtained as a pale yellow solid.

### Exemplary Synthesis of (2S,4R)-1-((S)-2-(2-(2-((1-((R)-2-(((S)-4-(4-acryloylpiperazin-1-yl)-6-chloro-8-fluoro-7-(3-hydroxynaphthalen-1-yl)quinazolin-2-yl)oxy)propyl)piperidin-4-yl)oxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide and (2S,4R)-1-((S)-2-(2-(2-((1-((R)-2-(((R)-4-(4-acryloylpiperazin-1-yl)-6-chloro-8-fluoro-7-(3-hydroxynaphthalen-1-yl)quinazolin-2-yl)oxy)propyl)piperidin-4-yl)oxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

### Step 1: Preparation of tert-butyl 4-(6-chloro-8-fluoro-2-(((R)-1-(4-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)piperidin-1-yl)propan-2-yl)oxy)-7-(3-hydroxynaphthalen-1-yl)quinazolin-4-yl)piperazine-1-carboxylate

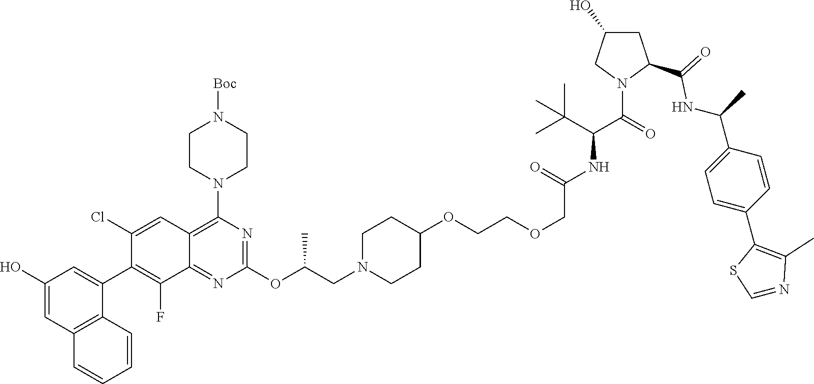 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc(N4CCN(C(=O)OC(C)(C)C)CC4)c4cc(Cl)c(-c5cc(O)cc6ccccc56)c(F)c4n3)CC2)C(C)(C)C)cc1]

To a solution of tert-butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (60 mg, 0.10 mmol, 1 eq) and (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (78 mg, 0.12 mmol, 1.2 eq) in methanol (1 mL) and dichloromethane (2 mL) was added acetic acid (12 mg, 0.20 mmol, 2 eq), then sodium cyanoborohydride (19 mg, 0.31 mmol, 3 eq) was added at 0° C. The mixture was stirred at 25° C. for 3 hours. The mixture was concentrated under vacuum. The mixture was purified by prep-TLC (Dichloromethane:Methanol=10:1) to get a product. Compound tert-butyl 4-[6-chloro-8-fluoro-2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]piperazine-1-carboxylate (63 mg) was obtained as a yellow solid. LC/MS (ESI) m/z: 1194.2 [M+1]⁺.

### Step 2: Preparation of (2S,4R)-1-((2S)-2-(2-(2-((1-((2R)-2-((6-chloro-8-fluoro-7-(3-hydroxynaphthalen-1-yl)-4-(piperazin-1-yl)quinazolin-2-yl)oxy)propyl)piperidin-4-yl)oxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

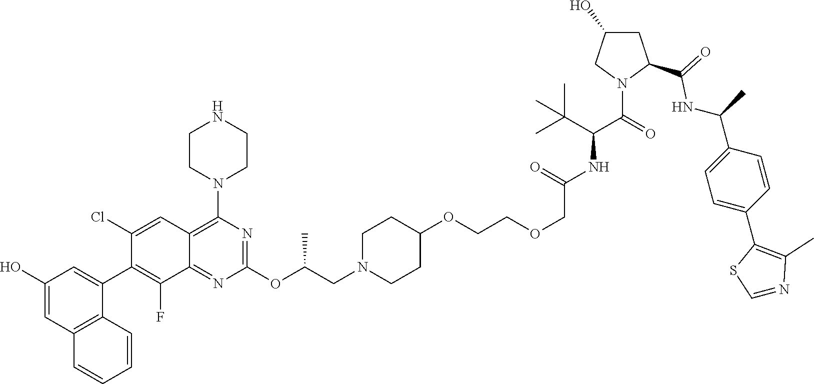 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc(N4CCNCC4)c4cc(Cl)c(-c5cc(O)cc6ccccc56)c(F)c4n3)CC2)C(C)(C)C)cc1]

To a solution of tert-butyl 4-[6-chloro-8-fluoro-2-[(1R)-2-[4-[2-[2-[[(1S)-1-[(2S,4R)-4- hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]piperazine-1-carboxylate (61 mg, 0.05 mmol, 1 eq) in dichloromethane (2 mL) was added trifluoroacetic acid (770 mg, 6.75 mmol, 0.5 mL, 132.28 eq). The mixture was stirred at 25° C. for 0.5 hour. The mixture was concentrated under vacuum. Compound (2S,4R)-1-[(2S)-2-[[2-[2-[[1-[(2R)-2-[6-chloro-8-fluoro-7-(3-hydroxy-1-naphthyl)-4- piperazin-1-yl-quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (61 mg, 0.05 mmol, 99% yield, trifluoroacetate) was obtained as a yellow oil.

### Step 3: Preparation of (2S,4R)-1-((2S)-2-(2-(2-((1-((2R)-2-((4-(4-acryloylpiperazin-1-yl)-6-chloro-8-fluoro-7-(3-hydroxynaphthalen-1-yl)quinazolin-2-yl)oxy)propyl)piperidin-4-yl)oxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

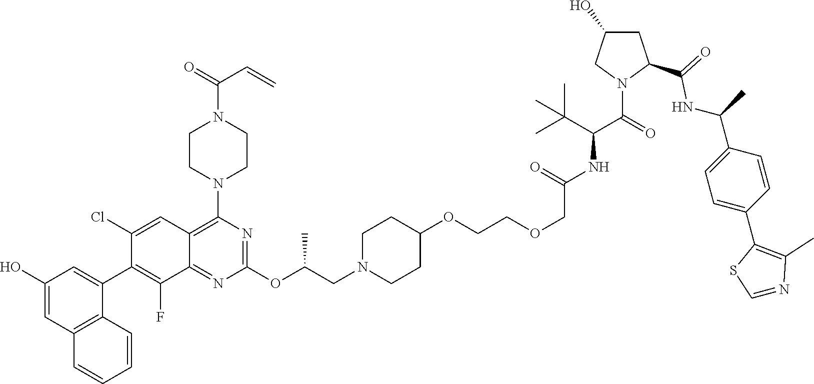 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To a solution of (2S,4R)-1-[(2S)-2-[[2-[2-[[1-[(2R)-2-[6-chloro-8-fluoro-7-(3-hydroxy-1-naphthyl)-4-piperazin-1-yl-quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (61 mg, 0.05 mmol, 1 eq, trifluoroacetate) in dichloromethane (2 mL) was added 2,6-lutidine (54 mg, 0.50 mmol, 10 eq), then prop-2-enoyl chloride (4 mg, 0.045 mmol, 0.9 eq) in dichloromethane (4 mL) was added at −65° C. The mixture was stirred at −65° C. for 10 minutes. Water (10 mL) was added. The aqueous phase was extracted with dichloromethane (15 mL*3). The combined organic phase was concentrated in vacuum. The residue was purified by semi-preparative reverse phase HPLC. Then the collected fraction was concentrated to remove most of the acetonitrile. The solution was lyophilized. Compound (2S,4R)-1-[(2S)-2-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (19 mg, 0.02 mmol, 31% yield, 99% purity, formate) as a white solid was obtained. LC/MS (ESI) m/z: 574.8 [M/2+1]⁺; ¹H-NMR (400 MHz, DMSO-d₆) δ 10.13-9.89 (m, 1H), 8.98 (s, 1H), 8.41 (br d, J=7.1 Hz, 1H), 8.26 (s, 1H), 8.00 (s, 1H), 7.80 (d, J=9.2 Hz, 1H), 7.42 (br d, J=7.0 Hz, 3H), 7.39-7.26 (m, 4H), 7.21 (br d, J=8.3 Hz, 2H), 7.06 (br d, J=5.4 Hz, 1H), 6.83 (dd, J=10.6, 16.9 Hz, 1H), 6.18 (br d, J=16.9 Hz, 1H), 5.74 (br d, J=10.6 Hz, 1H), 5.38 (br s, 1H), 5.12 (br s, 1H), 4.88 (br d, J=6.1 Hz, 1H), 4.52 (d, J=9.9 Hz, 1H), 4.43 (t, J=8.5 Hz, 1H), 4.27 (br s, 1H), 3.92 (br d, J=8.4 Hz, 6H), 3.85 (br s, 2H), 3.78 (br s, 2H), 3.54 (br d, J=14.1 Hz, 6H), 3.46-3.40 (m, 3H), 2.77 (br s, 2H), 2.61 (br s, 1H), 2.45 (s, 3H), 2.39 (br s, 1H), 2.15 (br s, 2H), 2.02 (br d, J=8.6 Hz, 1H), 1.77 (br s, 3H), 1.35 (br d, J=6.5 Hz, 3H), 1.30 (br d, J=6.1 Hz, 3H), 0.90 (br s, 9H).

### Step 4: Separation of Atropisomers of tert-Butyl 4-[6-chloro-8-fluoro-2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]piperazine-1-carboxylate

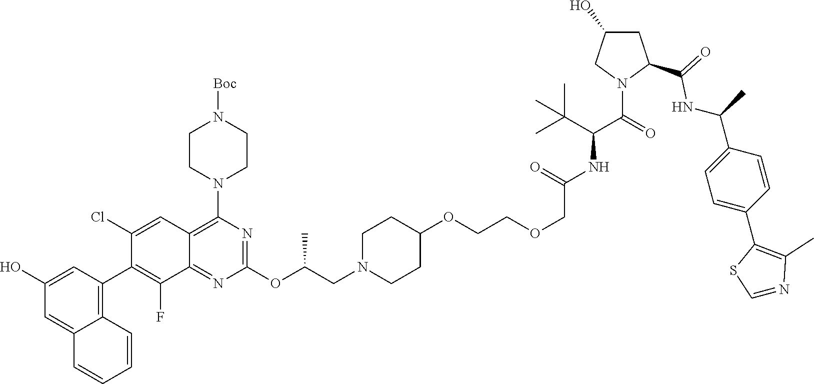 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc(N4CCN(C(=O)OC(C)(C)C)CC4)c4cc(Cl)c(-c5cc(O)cc6ccccc56)c(F)c4n3)CC2)C(C)(C)C)cc1]

The atropisomers of tert-butyl 4-[6-chloro-8-fluoro-2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]piperazine-1-carboxylate (105 mg, 0.088 mmol, 1 eq) were separated by SFC (60% isopropanol in 0.1% NH₄OH).

### Step 5: Preparation of (2S,4R)-1-[(2S)-2-[[2-[2-[[1-[(2R)-2-[6-chloro-8-fluoro-7-(3- hydroxy-1-naphthyl)-4-piperazin-1-yl-quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

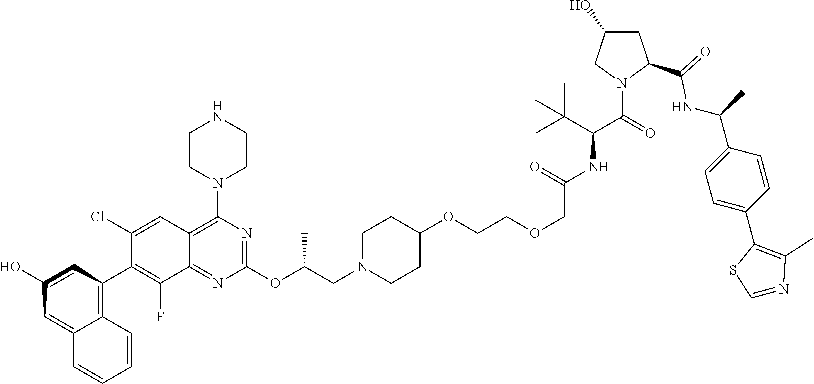

To a solution of tert-butyl 4-[6-chloro-8-fluoro-2-[(1R)-2-[4-[2-[2-[[(1S)-1-[(2S,4R)-4- hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]piperazine-1-carboxylate (56 mg, 0.047 mmol, 1 eq) in CH₂Cl₂ (2 mL) was added trifluoroacetic acid (383 mg, 3.36 mmol, 71.72 eq), and the reaction mixture was stirred at 20° C. for 1 hour. The reaction mixture was concentrated under vacuum to get (2S,4R)-1-[(2S)-2-[[2-[2-[[1-[(2R)-2-[6-chloro-8-fluoro-7-(3-hydroxy-1-naphthyl)-4-piperazin-1-yl- quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (56 mg, 0.046 mmol, 99% yield, TFA salt) as a yellow gum.

### Step 6: Preparation of (2S,4R)-1-[(2S)-2-[[2-[2-[[1-[(2R)-2-[6-chloro-8-fluoro-7-(3- hydroxy-1-naphthyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

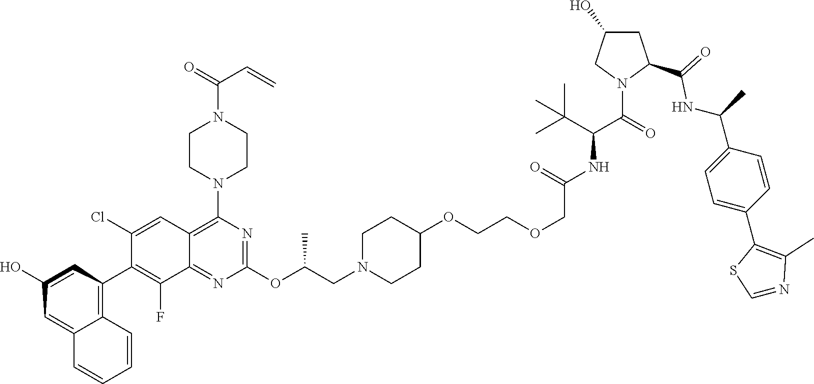

To a solution of (2S,4R)-1-[(2S)-2-[[2-[2-[[1-[(2R)-2-[6-chloro-8-fluoro-7-(3-hydroxy-1-naphthyl)-4-piperazin-1-yl-quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (56 mg, 0.046 mmol, 1 eq, TFA salt) and 2,6-lutidine (99 mg, 0.093 mmol, 20 eq) in CH₂Cl₂ (4 mL) at −70° C. prop-2-enoyl chloride (3.77 mg, 0.042 mmol, 0.9 eq) in CH₂Cl₂ (0.34 mL), and the reaction mixture was stirred at −70° C. for 10 minutes. The reaction was diluted with CH₂Cl₂ (30 mL) and water (20 mL). The organic layer was separated, and the aqueous layer was further extracted with CH₂Cl₂ (10 mL). The combined organic extracts were dried over Na₂SO₄ and concentrated. The resulting residue was purified by semi-preparative reverse phase HPLC (36-56% CH₃CN in water (0.1% TFA)) to get (2S,4R)-1-[(2S)-2-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (26.5 mg, 0.021 mmol, 45% yield, 99% purity, TFA salt) as a colorless gum. LC/MS (ESI) m/z: 1148.5 [M+H]⁺. ¹H-NMR (400 MHz, DMSO-d₆) δ 10.12 (s, 1H), 10.20-9.88 (m, 1H), 9.18 (s, 1H), 9.01-8.96 (m, 1H), 8.82-8.68 (m, 1H), 8.44-8.35 (m, 1H), 8.09-8.04 (m, 1H), 7.87-7.78 (m, 1H), 7.49-7.38 (m, 4H), 7.38-7.32 (m, 3H), 7.31-7.28 (m, 1H), 7.26-7.16 (m, 2H), 7.10-7.05 (m, 1H), 6.89-6.77 (m, 1H), 6.15 (s, 1H), 5.80-5.73 (m, 1H), 5.70-5.58 (m, 1H), 4.89 (br t, J=7.0 Hz, 1H), 4.54 (dd, J=7.0, 9.5 Hz, 1H), 4.44-4.39 (m, 1H), 4.32-4.22 (m, 2H), 3.96 (br d, J=5.3 Hz, 3H), 3.91 (br s, 2H), 3.87 (br d, J=1.0 Hz, 2H), 3.80 (br s, 2H), 3.66 (br s, 1H), 3.63-3.49 (m, 10H), 3.42-3.34 (m, 1H), 3.27-2.96 (m, 2H), 2.45 (s, 3H), 2.18-1.92 (m, 3H), 1.91-1.60 (m, 2H), 1.57-1.41 (m, 1H), 1.40-1.31 (m, 6H), 0.92 (br d, J=4.0 Hz, 9H).

The following opposite atropisomers was obtained in an analogous manner: (2S,4R)-1-[(2S)-2-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

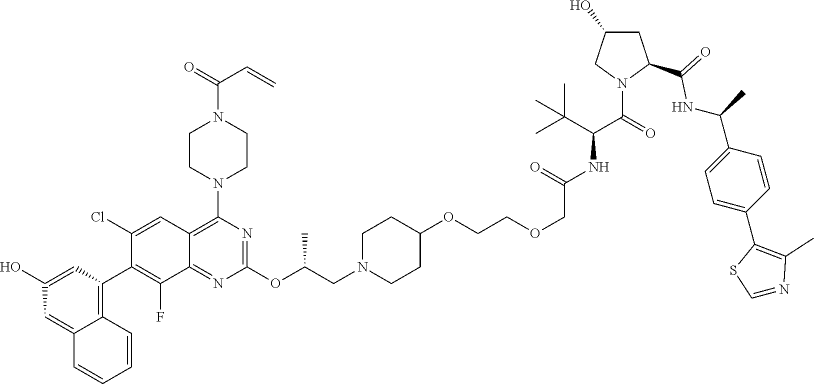

(TFA salt, colorless gum). LC/MS (ESI) m/z: 1148.5 [M+1]⁺. ¹H-NMR (400 MHz, DMSO-d₆) δ 10.06 (br s, 1H), 9.44-9.19 (m, 1H), 8.99 (s, 1H), 8.40 (s, 1H), 8.07 (s, 1H), 7.44 (br d, J=8.2 Hz, 3H), 7.39-7.10 (m, 6H), 7.08-7.04 (m, 1H), 6.89-6.77 (m, 1H), 6.24-6.14 (m, 1H), 5.80-5.73 (m, 1H), 5.72-5.56 (m, 1H), 4.89 (br t, J=6.6 Hz, 1H), 4.55 (br t, J=9.1 Hz, 1H), 4.45-4.40 (m, 2H), 4.29 (br s, 2H), 4.03-3.94 (m, 6H), 3.87 (br s, 2H), 3.80 (br s, 2H), 3.68 (br s, 1H), 3.64-3.49 (m, 8H), 3.43-3.34 (m, 1H), 3.27-2.97 (m, 2H), 2.45 (d, J=2.0 Hz, 3H), 2.20-2.11 (m, 1H), 2.11-1.87 (m, 3H), 1.78 (br s, 2H), 1.49-1.31 (m, 6H), 0.93 (br d, J=5.5 Hz, 9H).

### Exemplary of (2S,4R)-1-((S)-3,3-dimethyl-2-(2-(piperidin-4-ylmethoxy)acetamido)butanoyl)-4-hydroxy-N—((S)-1-(4-(4-methylthiazol-5-yl)phenyl)ethyl)pyrrolidine-2-carboxamide

### Step 1: Preparation of tert-butyl4-[(2-ethoxy-2-oxo-ethoxy)methyl]piperidine-1-carboxylate

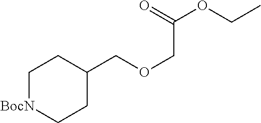 [SMILES: CCOC(=O)COCC1CCN(C(=O)OC(C)(C)C)CC1]

To a solution of tert-butyl 4-(hydroxymethyl)piperidine-1-carboxylate (2.00 g, 9.29 mmol, 1.00 eq) in CH₂Cl₂ (50 mL) were added diacetoxyrhodium (1.20 g, 4.64 mmol, 0.50 eq) and ethyl 2-diazoacetate (12.00 g, 92.90 mmol, 10 mL, 10.00 eq) at 0° C., and the reaction mixture was stirred at 25° C. for 12 hours. The solution was concentrated under reduced pressure and dried under vacuum. Purification by column chromatography on SiO₂ (0-20% EtOAc in petroleum ether) afforded tert-butyl 4-[(2-ethoxy-2-oxo-ethoxy)methyl]piperidine-1-carboxylate (1.50 g, 4.98 mmol, 54% yield) as a yellow oil. LC/MS (ESI) m/z: 202.2 [M-Boc+1]⁺.

### Step 2: Preparation of 2-[(1-tert-butoxycarbonyl-4-piperidyl)methoxy]acetic Acid

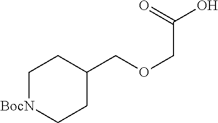 [SMILES: CC(C)(C)OC(=O)N1CCC(COCC(=O)O)CC1]

To a solution of tert-butyl 4-[(2-ethoxy-2-oxo-ethoxy)methyl]piperidine-1-carboxylate (1.50 g, 4.98 mmol, 1.00 eq) in THF (10 mL), CH₃OH (5 mL) and H₂O (5 mL) was added LiOH hydrate (700 mg, 14.93 mmol, 3.00 eq), and the reaction mixture was stirred at 25° C. for 12 hours. The reaction was acidified (pH=5) with dilute hydrochloric acid, and the resulting mixture was concentrated under reduced pressure to afford 2-[(1-tert-butoxycarbonyl-4-piperidyl)methoxy]acetic acid (1.00 g, 3.66 mmol, 74% yield) as a yellow oil.

### Step 3: Preparation of tert-butyl4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]piperidine-1-carboxylate

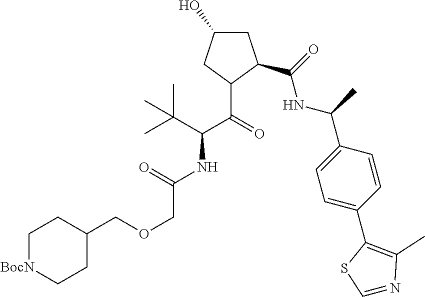 [SMILES: Cc1ncsc1-c1ccc([C@H](C)NC(=O)[C@@H]2C[C@@H](O)CC2C(=O)[C@@H](NC(=O)COCC2CCN(C(=O)OC(C)(C)C)CC2)C(C)(C)C)cc1]

To a solution of 2-[(1-tert-butoxycarbonyl-4-piperidyl)methoxy]acetic acid (115 mg, 0.42 mmol, 1.00 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (200 mg, 0.42 mmol, 1.00 eq, hydrochloride salt) in CH₂Cl₂ (4 mL) was added hydroxybenzotriazole (85 mg, 0.62 mmol, 1.50 eq), 1-(3-dimethylaminopropyl)-3-ethylcarbodiimide hydrochloride (120 mg, 0.62 mmol, 1.50 eq), and diisopropylethylamine (270 mg, 2.08 mmol, 5.00 eq), and the reaction mixture was stirred at 25° C. for 12 hours. The solution was concentrated under reduced pressure and dried under vacuum. Purification by thin layer chromatography (EtOAc/CH₃OH=20/1) afforded tert-butyl4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]piperidine-1-carboxylate (210 mg, 0.30 mmol, 72% yield) as a yellow oil. LC/MS (ESI) m/z: 700.4 [M+H]⁺.

### Step 4: Preparation of (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide

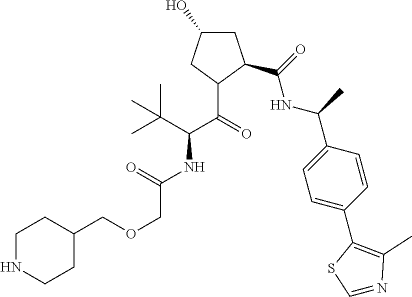 [SMILES: Cc1ncsc1-c1ccc([C@H](C)NC(=O)[C@@H]2C[C@@H](O)CC2C(=O)[C@@H](NC(=O)COCC2CCNCC2)C(C)(C)C)cc1]

To a solution of tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]piperidine-1-carboxylate (100 mg, 0.14 mmol, 1.00 eq) in CH₂Cl₂ (2 mL) was added HCl (4M in dioxane, 2 mL), and the reaction mixture was stirred at 25° C. for 0.5 hour. . The solution was concentrated under reduced pressure and dried under vacuum to afford (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (90 mg, 0.14 mmol, 99% yield, hydrochloride salt) was obtained as a yellow solid.

### Exemplary Synthesis of tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

### Step 1: Preparation of tert-butyl 4-[[5-(1-methoxycarbonyl-2-methyl-propyl) isoxazol-3-yl]oxymethyl]piperidine-1-carboxylate

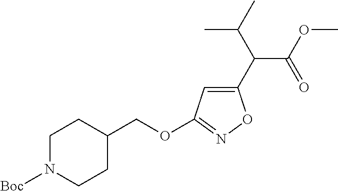 [SMILES: COC(=O)C(c1cc(OCC2CCN(C(=O)OC(C)(C)C)CC2)no1)C(C)C]

To a solution of tert-butyl 4-(hydroxymethyl) piperidine-1-carboxylate (1.30 g, 6.02 mmol, 1.2 eq) and methyl 2-(3-hydroxyisoxazol-5-yl)-3-methyl-butanoate (1 g, 5.02 mmol, 1 eq) in THP (10 mL) were added Ph₃P (1.58 g, 6.02 mmol, 1.2 eq) and diisopropyl azodicarboxylate (1.22 g, 6.02 mmol, 1.17 mL, 1.2 eq), and the reaction mixture was stirred at 25° C. for 12 hours. The reaction mixture was concentrated under reduced pressure, and the resulting residue was purified by flash silica gel chromatography (0-60% EtOAc in petroleum ether) to afford tert-butyl 4-[[5-(1-methoxycarbonyl-2-methyl-propyl) isoxazol-3-yl]oxymethyl]piperidine-1-carboxylate (1.8 g, 4.54 mmol, 90% yield) as a white solid. LC/MS (ESI) m/z: 297.2 [M+H]⁺; ¹H-NMR (400 MHz, CDCl₃) δ 5.88 (s, 1H), 4.21-4.10 (m, 2H), 4.07 (d, J=6.4 Hz, 2H), 3.73 (s, 3H), 3.49 (d, J=8.7 Hz, 1H), 2.82-2.62 (m, 2H), 2.35 (qd, J=7.1, 14.2 Hz, 1H), 1.96 (br d, J=3.4 Hz, 1H), 1.77 (br d, J=12.8 Hz, 2H), 1.46 (s, 9H), 1.29-1.22 (m, 2H), 1.00 (d, J=6.7 Hz, 3H), 0.93 (d, J=6.7 Hz, 3H).

### Step 2: Preparation of 2-[3-[(1-tert-butoxycarbonyl-4-piperidyl) methoxy]isoxazol-5-yl]-3-methyl-butanoic Acid

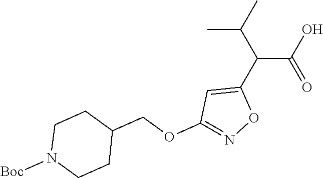 [SMILES: CC(C)C(C(=O)O)c1cc(OCC2CCN(C(=O)OC(C)(C)C)CC2)no1]

To a solution of tert-butyl 4-[[5-(1-methoxycarbonyl-2-methyl-propyl) isoxazol-3-yl]oxymethyl]piperidine-1-carboxylate (1.8 g, 4.54 mmol, 1 eq) in THF (8 mL), CH₃OH (5 mL), and H₂O (3 mL) was added LiOH monohydrate (544 mg, 22.70 mmol, 5 eq), and the reaction mixture was stirred at 25° C. for 1 hours. The reaction mixture was acidified (pH=3) by addition of 1M hydrochloric acid, and the resulting precipitate was filtered to afford crude 2-[3-[(1-tert-butoxycarbonyl-4-piperidyl) methoxy]isoxazol-5-yl]-3-methyl-butanoic acid (1.9 g) as a white solid.

### Step 3: Preparation of tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

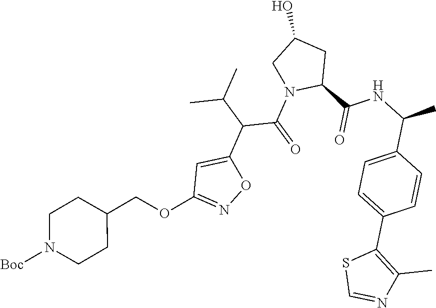 [SMILES: Cc1ncsc1-c1ccc([C@H](C)NC(=O)[C@@H]2C[C@@H](O)CN2C(=O)C(c2cc(OCC3CCN(C(=O)OC(C)(C)C)CC3)no2)C(C)C)cc1]

To a solution of (2S,4R)-4-hydroxy-N-[(1S)-1-[4-(4-methylthiazol-5-yl) phenyl]ethyl]pyrrolidine-2-carboxamide (96 mg, 0.26 mmol, 1 eq, HCl salt) and 2-[3-[(1-tert-butoxycarbonyl-4-piperidyl) methoxy]isoxazol-5-yl]-3-methyl-butanoic acid (100 mg, 0.26 mmol, 1 eq) in DMF (2 mL) were added diisopropylethylamine (101 mg, 0.78 mmol, 137 uL, 3 eq) and O-(7-azabenzotriazol-1-yl)-N,N,N,N-tetramethyluronium hexafluorophosphate (149 mg, 0.39 mmol, 1.5 eq) at 0° C., and the reaction mixture was stirred at 20° C. for 1 hour. The mixture was partitioned between H₂O and EtOAc. The organic phase was separated, washed with brine, dried over Na₂SO₄, filtered, and concentrated under reduced pressure. The resulting residue was purified by prep-TLC (SiO₂, CH₂Cl₂:CH₃OH=10:1) to afford tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate (130 mg, 0.18 mmol, 70% yield, 98% purity) as a yellow solid. LC/MS (ESI) m/z: 696.3 [M+H]⁺. This material was separated by SFC to afford tert-butyl4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate and tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate .

¹H NMR: tert-butyl4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

¹H-NMR (400 MHz, CDCl₃) δ 8.68 (s, 1H), 7.48 (br d, J=7.5 Hz, 1H), 7.39 (q, J=8.3 Hz, 4H), 5.88 (s, 1H), 5.07 (t, J=7.2 Hz, 1H), 4.67-4.60 (m, 2H), 4.19-4.02 (m, 4H), 3.80 (dd, J=5.1, 10.5 Hz, 1H), 3.60 (dd, J=3.7, 10.4 Hz, 1H), 3.53-3.48 (m, 1H), 2.78-2.66 (m, 2H), 2.58-2.47 (m, 4H), 2.46-2.35 (m, 1H), 1.96 (ddd, J=5.0, 8.0, 12.8 Hz, 2H), 1.75 (br d, J=12.1 Hz, 3H), 1.50 (d, J=7.0 Hz, 3H), 1.46 (s, 9H), 1.28-1.19 (m, 2H), 1.05 (d, J=6.6 Hz, 3H), 0.93 (d, J=6.7 Hz, 3H).

¹H NMR: tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

¹H-NMR (400 MHz, CDCl₃) δ 8.70-8.68 (m, 1H), 7.42-7.39 (m, 2H), 7.36-7.32 (m, 3H), 5.86 (s, 1H), 5.02-4.94 (m, 1H), 4.78 (dd, J=4.3, 8.3 Hz, 1H), 4.66 (quin, J=5.4 Hz, 1H), 4.16-4.09 (m, 1H), 4.08-4.01 (m, 2H), 3.74-3.68 (m, 1H), 3.60-3.48 (m, 2H), 2.78-2.62 (m, 3H), 2.52-2.41 (m, 1H), 2.02-1.93 (m, 2H), 1.75 (br d, J=9.9 Hz, 6H), 1.46 (s, 9H), 1.38 (d, J=7.0 Hz, 3H), 1.30-1.18 (m, 3H), 1.06 (d, J=6.6 Hz, 3H), 0.94 (d, J=6.7 Hz, 3H).

### Exemplary Synthesis of (2S,4R)-1-[(2S)-2-[[2-[2-[[1-[(2R)-2-[6-chloro-8-[(5-chloro-6-fluoro-1H-indazol-4-yl)oxy]-4-(4-prop-2-enoylpiperazin-1-yl)pyrido[3,4-d]pyrimidin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 2,6-dichloro-3-fluoro-pyridine-4-carboxylic Acid

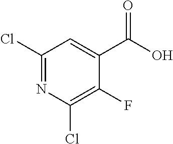 [SMILES: O=C(O)c1cc(Cl)nc(Cl)c1F]

To a solution of 2,6-dichloro-3-fluoro-pyridine (14.2 g, 85.55 mmol, 1 eq) in THF (200 mL) cooled at −78° C. was added n-butyllithium (2.5 M, 41.07 mL, 1.2 eq) dropwise, and the reaction mixture was stirred at −78° C. for 1 hour. CO₂ (4.52 g, 102.66 mmol, dry ice, 1.2 eq) was added in portions, and the reaction mixture was stirred at −78° C. for another 2 hours. Saturated aqueous NH₄Cl (30 mL) was added followed by 1N aqueous H2504 until the mixture was neutralized (pH=7). The resulting mixture was extracted 2-methoxy-2-methylpropane (2×50 mL), and the combined organic extracts were concentrated under vacuum. The aqueous extract was acidified (pH=1) by addition of 1N aqueous H2SO₄. The resulting mixture was extracted with EtOAc (2×150 mL), and the combined organic extracts were dried over Na₂SO₄ and concentrated under vacuum to get 2,6-dichloro-3-fluoro-pyridine-4-carboxylic acid (14 g, 66.67 mmol, 78% yield) as a yellow solid. ¹H-NMR (400 MHz, DMSO-d₆) δ 7.85 (d, J=4.4, 1H).

### Step 2: Preparation of 2,6-dichloro-3-fluoro-pyridine-4-carbonyl Chloride

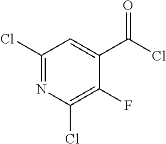 [SMILES: O=C(Cl)c1cc(Cl)nc(Cl)c1F]

To a solution of 2,6-dichloro-3-fluoro-pyridine-4-carboxylic acid (12 g, 57.15 mmol, 1 eq) in thionyl chloride (78.72 g, 661.68 mmol, 48.00 mL, 11.58 eq) was added DMF (0.02 g, 0.27 mmol, 4.79e-3 eq), and the reaction mixture was stirred at 80° C. for 3 hours. The reaction mixture was concentrated under vacuum, and the resulting residue was taken up in toluene (50 mL), and the mixture concentrated to get the 2,6-dichloro-3-fluoro-pyridine-4-carbonyl chloride (13 g, 56.91 mmol, 99% yield) as a yellow gum.

### Step 3: Preparation of 2,6-dichloro-3-fluoro-N-(methylsulfanylcarbonimidoyl)pyridine-4-carboxamide

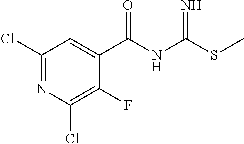 [SMILES: CSC(=N)NC(=O)c1cc(Cl)nc(Cl)c1F]

To a solution of NaOH (10.24 g, 256.09 mmol, 4.5 eq) in H₂O (200 mL) was added 2-methylisothiourea (15 g, 79.69 mmol, 1.40 eq, sulfate) in portions at 0° C., and the resulting mixture was stirred for 10 minutes. A solution of 2,6-dichloro-3-fluoro-pyridine-4-carbonyl chloride (13 g, 56.91 mmol, 1 eq) in THF (150 mL) at 0-5° C. was then added dropwise, and the reaction mixture was stirred at 0-5° C. for 30 minutes. EtOAc (150 mL) and H₂O (200 mL) were added, the layers separated, and the aqueous layer was further extracted with EtOAc (2×50 mL). The combined organic extracts were washed with H₂O (2×80 mL) followed by brine (100 mL), dried over Na₂SO₃, and concentrated to afford crude 2,6-dichloro-3-fluoro-N-(methylsulfanylcarbonimidoyl)pyridine-4-carboxamide (16 g) as a yellow solid.

### Step 4: Preparation of 6,8-dichloro-2-methylsulfanyl-3H-pyrido[3,4-d]pyrimidin-4-one

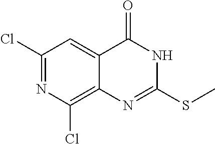

To a solution of 2,6-dichloro-3-fluoro-N-(methylsulfanylcarbonimidoyl)pyridine-4-carboxamide (16 g, 56.71 mmol, 1 eq) in DMF (100 mL) was added Cs₂CO₃ (25.87 g, 79.40 mmol, 1.4 eq), and the reaction mixture was stirred at 90° C. for 5 hours. The mixture was cooled to 25° C., diluted with H₂O (200 mL), and acidified pH=6) by addition of 3 M aqueous acetic acid. The resulting precipitate was collected by filtration, and the filter cake was washed with H₂O (3×50 mL), then dried under vacuum. The resulting crude product was purified by SiO₂ column chromatography (0-20% EtOAc in petroleum ether) to afford 6,8-dichloro-2-methylsulfanyl-3H-pyrido[3,4-d]pyrimidin-4-one (2.8 g, 10.68 mmol, 19% yield) as a yellow solid. ¹H-NMR (400 MHz, CDCl₃) δ 13.21 (s, 1H), 7.85 (s, 1H), 1.45 (s, 9H), 2.61 (s, 3H).

### Step 5: Preparation of 4,6,8-trichloro-2-methylsulfanyl-pyrido[3,4-d]pyrimidine

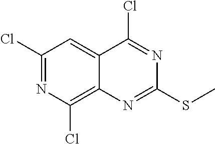

A mixture of 6,8-dichloro-2-methylsulfanyl-3H-pyrido[3,4-d]pyrimidin-4-one (2.5 g, 9.54 mmol, 1 eq) and POCl₃ (30.94 g, 201.77 mmol, 18.75 mL, 21.15 eq) was heated at 130° C. for 3 hours. The mixture was cooled to 25° C. and then concentrated under vacuum. The resulting residue was dissolved in EtOAc (30 mL), and the resulting organic mixture was washed with water (30 mL), dried over Na₂SO₄, and concentrated under vacuum. The resulting residue was purified by SiO₂ column chromatography (0-20% EtOAc in petroleum ether) to afford 4,6,8-trichloro-2-methylsulfanyl-pyrido[3,4-d]pyrimidine (750 mg, 2.67 mmol, 28% yield) as a yellow solid.

### Step 6: Preparation of tert-butyl 4-(6,8-dichloro-2-methylsulfanyl-pyrido[3,4-d]pyrimidin-4-yl)piperazine-1-carboxylate

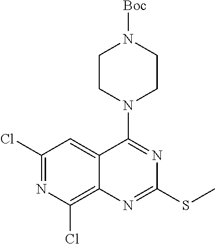 [SMILES: CSc1nc(N2CCN(C(=O)OC(C)(C)C)CC2)c2cc(Cl)nc(Cl)c2n1]

To a solution of 4,6,8-trichloro-2-methylsulfanyl-pyrido[3,4-d]pyrimidine (750 mg, 2.67 mmol, 1 eq) and triethylamine (541 mg, 5.35 mmol, 2 eq) in CH₂Cl₂ (12 mL) was added tert-butyl piperazine-1-carboxylate (448 mg, 2.41 mmol, 0.9 eq), and the reaction mixture was stirred at 20° C. for 3 hours. The mixture was concentrated under vacuum, and the resulting residue was purified by flash chromatography on SiO₂ (0-10% EtOAc in petroleum ether) to get tert-butyl 4-(6,8-dichloro-2-methylsulfanyl-pyrido[3,4-d]pyrimidin-4-yl)piperazine-1-carboxylate (680 mg, 1.58 mmol, 59% yield) as a yellow gum.

### Step 7: Preparation of 3-bromo-5-fluoro-2-methyl-aniline

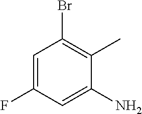

To a mixture of 1-bromo-5-fluoro-2-methyl-3-nitro-benzene (15 g, 64.10 mmol, 1 eq) and NH₄Cl (17.14 g, 320.48 mmol, 5 eq) in H₂O (30 mL) and ethanol (150 mL) at 80° C. was added Fe (17.90 g, 320.48 mmol, 5 eq) in portions, and the reaction mixture was stirred at 80° C. for 14 hours. The mixture was filtered, and the filtrate was extracted with EtOAc (3×20 mL). The combined organic extracts were washed with brine (3×20 mL), dried over anhydrous Na₂SO₄, filtered, and concentrated in vacuum. The resulting residue was purified by column chromatography on SiO₂ (0-100% EtOAc in petroleum ether) to afford the desired product (4.593 g). Additional (4.684 g) material was obtained by further purification of impure fractions by semi-preparative reverse phase HPLC (30-60% CH₃CN in water (0.1% TFA)). Compound 3-bromo-5-fluoro-2-methyl-aniline (9.28 g, 45.34 mmol, 71% yield, 99% purity) was obtained as a brown oil. ¹H-NMR (400 MHz, CDCl₃) δ 7.75 (dd, J=2.4, 10.0 Hz, 1H), 4.48 (s, 2H), 2.20 (s, 3H).

### Step 8: Preparation of 3-bromo-4-chloro-5-fluoro-2-methylaniline

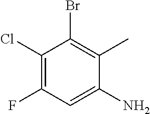

To a solution of 3-bromo-5-fluoro-2-methyl-aniline (9.28 g, 45.47 mmol, 1 eq) in isopropanol (50 mL) was added 1-chloropyrrolidine-2,5-dione (6.68 g, 50.01 mmol, 1.1 eq), and the reaction mixture was stirred at 80° C. for 2 hours. The reaction mixture was concentrated under reduced pressure, and the resulting residue was purified by flash chromatography on SiO₂ (10-30% EtOAc in petroleum ether) followed by purification on semi-preparative reverse phase HPLC [45-75% CH₃CN in water (0.05% HCl)) to afford 3-bromo-4-chloro-5-fluoro-2-methyl-aniline (4.87 g, 20.36 mmol, 45% yield, 99% purity) as a white solid. ¹H-NMR (400 MHz, DMSO-d₆) δ 6.62 (d, J=11.6 Hz, 1H), 2.18 (m, 3H).

### Step 9: Preparation of 4-bromo-5-chloro-6-fluoro-1H-indazole

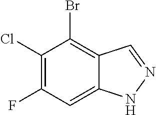 [SMILES: Fc1cc2[nH]ncc2c(Br)c1Cl]

To a solution of 3-bromo-4-chloro-5-fluoro-2-methyl-aniline (4.87 g, 20.42 mmol, 1 eq) in acetic acid (40 mL) was added NaNO₂ (1.80 g, 26.14 mmol, 1.28 eq), and the reaction mixture was stirred at 25° C. for 7 hours followed by 14 hours at 40° C. The mixture was diluted with water (200 mL) and extracted with EtOAc (3×50 mL). The combined organic extracts were washed with brine (3×30 mL), dried over anhydrous Na₂SO₄, filtered, and concentrated under reduced pressure. The resulting residue was purified by column chromatography on SiO₂ (10-100% CH₂Cl₂ in petroleum ether). The product was washed with CH₂Cl₂ (100 mL) to afford pure 4-bromo-5-chloro-6-fluoro-1H-indazole (751 mg, 3.01 mmol, 14.74% yield) and additional crude product (1.42 g). ¹H-NMR (400 MHz, DMSO-d₆) δ 13.68 (s, 1H), 8.10 (s, 1H), 7.70 (d, J=9.2 Hz, 1H).

### Step 10: Preparation of 4-bromo-5-chloro-6-fluoro-1-tetrahydropyran-2-yl-indazole

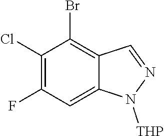

To a solution of 4-bromo-5-chloro-6-fluoro-1H-indazole (1.2 g, 4.81 mmol, 1 eq) in CH₂Cl₂ (50 mL) were added p-toluene sulfonic acid (92 mg, 0.48 mmol, 0.1 eq) and 3,4-dihydro-2H-pyran (809 mg, 9.62 mmol, 2 eq), and the reaction mixture was stirred at 20° C. for 0.5 hours. The mixture was diluted with H₂O (30 mL) and extracted with EtOAc (3×20 mL). The combined organic extracts were washed with brine (3×30 mL), dried over anhydrous Na₂SO₄, filtered, and concentrated under reduced pressure. The resulting residue was purified by column chromatography on SiO₂ (3-10% EtOAc in petroleum ether) to afford 4-bromo-5-chloro-6-fluoro-1-tetrahydropyran-2-yl-indazole (1.26 g, 3.78 mmol, 78% yield) as a yellow oil. ¹H-NMR (400 MHz, CDCl₃) δ 7.86 (s, 1H), 7.27 (d, J=9.2 Hz, 1H), 5.53 (dd, J=2.4, 9.2 Hz, 1H), 3.87-3.85 (m, 1H), 3.65-3.60 (m, 1H), 2.36-2.33 (m, 1H), 2.04-2.00 (m, 2H), 1.65-1.57 (m, 3H).

### Step 11: Preparation of 5-chloro-6-fluoro-1-(tetrahydro-2H-pyran-2-yl)-1H-indazol-4-ol

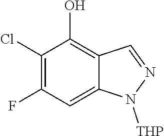

To 4-bromo-5-chloro-6-fluoro-1-tetrahydropyran-2-yl-indazole (2.24 g, 6.71 mmol, 1 eq) in dioxane (30 mL) were added tris(dibenzylideneacetone)dipalladium(O) (307 mg, 0.34 mmol, 0.05 eq), ditert-butyl-[2-(2,4,6-triisopropylphenyl)phenyl]phosphane (285 mg, 0.67 mmol, 0.1 eq) followed by KOH (1.13 g, 20.14 mmol, 3 eq) in H₂O (8 mL), and the reaction mixture was degassed and charged with N₂ (3×), then stirred at 90° C. for 16 hours under N₂ atmosphere. The resulting residue was partitioned between petroleum ether (50 mL) and water. The aqueous layer was extracted with petroleum ether (3×30 mL) and the combined organic extracts were discarded. The aqueous phase was acidified (pH=3) by addition of 1N aqueous HCl, then extracted with EtOAc (3×30 mL). The combined organic extracts were washed with water (50 mL) followed by brine (100 mL), dried over Na₂SO₄, and concentrated to dryness. The resulting residue was purified by flash chromatography on SiO₂ (0-20% EtOAc in petroleum ether) to afford 5-chloro-6-fluoro-1-tetrahydropyran-2-yl-indazol-4-ol (1.44 g, 5.31 mmol, 79% yield) as a yellow solid. ¹H-NMR (400 MHz, CDCl₃) δ 8.08 (s, 1H), 7.07 (d, J=0.8 Hz, 1H), 6.31 (s, 1H), 5.60 (dd, J=2.4, 9.2 Hz, 1H), 4.14-4.00 (m, 1H), 3.74-3.73 (m, 1H), 2.51-2.48 (m, 1H), 2.14-2.06 (m, 2H), 1.77-1.67 (m, 3H).

### Step 12: Preparation of tert-butyl 4-[6-chloro-8-(5-chloro-6-fluoro-1-tetrahydropyran-2-yl-indazol-4-yl)oxy-2-methylsulfanyl-pyrido[3,4-d]pyrimidin-4-yl]piperazine-1-carboxylate

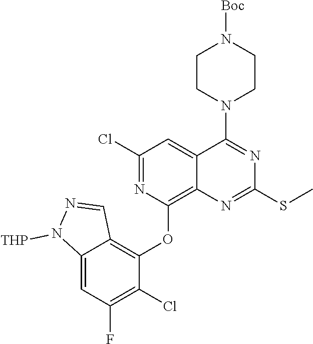

To a mixture of 5-chloro-6-fluoro-1-tetrahydropyran-2-yl-indazol-4-ol (300 mg, 1.11 mmol, 1 eq) and tert-butyl 4-(6,8-dichloro-2-methylsulfanyl-pyrido[3,4-d]pyrimidin-4-yl)piperazine-1-carboxylate (501 mg, 1.16 mmol, 1.05 eq) in dry DMA (6 mL) was added Cs₂CO₃ (541.65 mg, 1.66 mmol, 1.5 eq), and the reaction mixture was stirred at 85° C. for 5 hours. The mixture was cooled to 25° C. and then concentrated under vacuum. The remaining residue was dissolved in EtOAc (40 mL), and the resulting organic mixture was washed with water (30 mL), dried over Na₂SO₄, and concentrated under vacuum. The resulting residue was purified by column chromatography on SiO₂ (0-20% EtOAc in petroleum ether) to get tert-butyl 4-[6-chloro-8-(5-chloro-6-fluoro-1-tetrahydropyran-2-yl-indazol-4-yl)oxy-2-methylsulfanyl-pyrido[3,4-d]pyrimidin-4-yl]piperazine-1-carboxylate (420 mg, 0.63 mmol, 57% yield) as a yellow solid. ¹H-NMR (400 MHz, CDCl₃) δ 7.87 (s, 1H), 7.34 (dd, J=0.8, 8.4 Hz, 1H), 7.32 (s, 1H), 4.08-4.04 (m, 1H), 3.84-3.82 (m, 4H), 3.79-3.76 (m, 1H), 3.69-3.66 (m, 4H), 2.60 (s, 3H), 2.70-2.48 (m, 1H), 2.17-2.13 (m, 1H), 1.79-1.71 (m, 3H), 1.52 (s, 9H).

### Step 13: Preparation of tert-butyl 4-(6-chloro-8-((5-chloro-6-fluoro-1-(tetrahydro-2H-pyran-2-yl)-1H-indazol-4-yl)oxy)-2-(methylsulfonyl)pyrido[3,4-d]pyrimidin-4-yl)piperazine-1-carboxylate

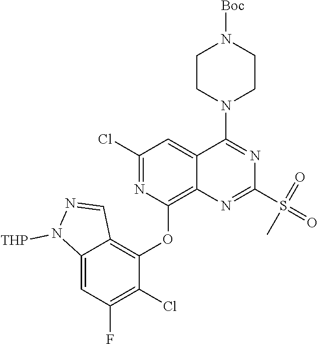

To a solution of tert-butyl 4-[6-chloro-8-(5-chloro-6-fluoro-1-tetrahydropyran-2-yl-indazol-4-yl)oxy-2-methylsulfanyl-pyrido[3,4-d]pyrimidin-4-yl]piperazine-1-carboxylate (320 mg, 0.48 mmol, 1 eq) in CH₂Cl₂ (8 mL) was added 3-chlorobenzoperoxoic acid (196 mg, 0.96 mmol, 85% purity, 2 eq), and the reaction mixture was stirred at 20° C. for 16 hours. The mixture was diluted with CH₂Cl₂ (30 mL), and the resulting organic mixture was washed with water (30 mL), dried over Na₂SO₄, and concentrated under vacuum. The resulting residue was purified by prep-TLC (80% EtOAc in petroleum ether) to get tert-butyl 4-[6-chloro-8-(5-chloro-6-fluoro-1-tetrahydropyran-2-yl-indazol-4-yl)oxy-2-methylsulfonyl-pyrido[3,4-d]pyrimidin-4-yl]piperazine-1-carboxylate (230 mg, 0.33b mmol, 68% yield) as a yellow solid. LC/MS (ESI) m/z: 696.1 [M+H]⁺.

### Step 13: Preparation of tert-butyl 4-[6-chloro-8-(5-chloro-6-fluoro-1-tetrahydropyran-2-yl-indazol-4-yl) oxy-2-[(1R)-2,2-dimethoxy-1-methylethoxy]pyrido[3,4-d]pyrimidin-4-yl]piperazine-1-carboxylate

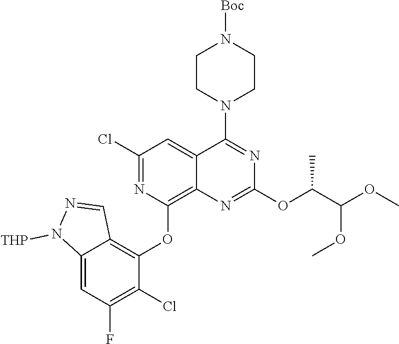

To a solution of tert-butyl 4-[6-chloro-8-(5-chloro-6-fluoro-1-tetrahydropyran-2-yl-indazol-4-yl) oxy-2-methylsulfonyl-pyrido[3,4-d]pyrimidin-4-yl]piperazine-1-carboxylate (100 mg, 0.14 mmol, 1 eq) and (2R)-1,1-dimethoxypropan-2-ol (35 mg, 0.29 mmol, 2 eq) in THF (3 mL) at 0° C. was added LiHMDS (1 M, 0.22, 1.5 eq), and the reaction mixture was stirred at 0-5° C. for 30 minutes. Saturated aqueous NH₄Cl (15 mL) and EtOAc (20 mL) were added, and the organic layer was separated, dried over Na₂SO₄, and concentrated under vacuum. The resulting residue was purified by prep-TLC (33% EtOAc in petroleum ether) to get tert-butyl 4-[6-chloro-8-(5-chloro-6-fluoro-1-tetrahydropyran-2-yl-indazol-4-yl)oxy-2-[(1R)-2,2-dimethoxy-1-methylethoxy]pyrido[3,4-d]pyrimidin-4-yl]piperazine-1-carboxylate (70 mg, 0.095 mmol, 66% yield) as a yellow solid.

### Step 14: Preparation of (R)-2-((6-chloro-8-((5-chloro-6-fluoro-1H-indazol-4-yl)oxy)-4-(piperazin-1-yl)pyrido[3,4-d]pyrimidin-2-yl)oxy)propanal

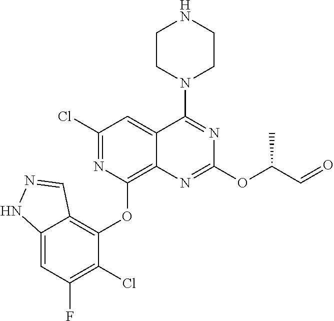 [SMILES: C[C@H](C=O)Oc1nc(N2CCNCC2)c2cc(Cl)nc(Oc3c(Cl)c(F)cc4[nH]ncc34)c2n1]

To a solution of tert-butyl 4-[6-chloro-8-(5-chloro-6-fluoro-1-tetrahydropyran-2-yl-indazol-4-yl) oxy-2-[(1R)-2,2-dimethoxy-1-methyl-ethoxy]pyrido[3,4-d]pyrimidin-4-yl]piperazine-1-carboxylate (70 mg, 0.095 mmol, 1 eq) in dioxane (3 mL) was added aqueous HCl (12 M, 0.5 mL, 63.14 eq), and the reaction mixture was stirred at 15° C. for 1 hour. The mixture was concentrated under vacuum to get (R)-2-((6-chloro-8-((5-chloro-6-fluoro-1H-indazol-4-yl)oxy)-4-(piperazin-1-yl)pyrido[3,4-d]pyrimidin-2-yl)oxy)propanal (61 mg, 0.095 mmol, 99% yield, hydrochloride) as a yellow solid. LC/MS (ESI) m/z: 505.9 [M+H]⁺.

### Step 15: Preparation of (2R)-2-[6-chloro-8-[(5-chloro-6-fluoro-1H-indazol-4-yl)oxy]-4-(4-prop-2-enoylpiperazin-1-yl) pyrido[3,4-d]pyrimidin-2-yl]oxypropanal

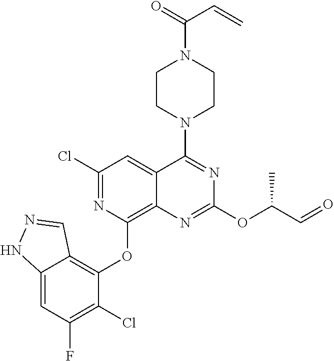 [SMILES: C=CC(=O)N1CCN(c2nc(O[C@H](C)C=O)nc3c(Oc4c(Cl)c(F)cc5[nH]ncc45)nc(Cl)cc23)CC1]

To a mixture of (2R)-2-[6-chloro-8-[(5-chloro-6-fluoro-1H-indazol-4-yl) oxy]-4-piperazin-1-yl-pyrido[3,4-d]pyrimidin-2-yl]oxypropanal (61 mg, 0.11 mmol, 1 eq, hydrochloride) and NaHCO₃ (6.48 g, 3 mL) in THF (3 mL) was added a solution of prop-2-enoyl chloride (10 mg, 0.11 mmol, 9.16 uL, 1 eq) in THF (0.9 mL), and the reaction mixture was stirred at 15° C. for 20 minutes. EtOAc (20 mL) and water (20 mL) were added, and the organic layer was separated, dried over Na₂SO₄, and concentrated under vacuum. The resulting residue was purified by prep-TLC (10% CH₃OH in CH₂Cl₂) to afford (2R)-2-[6-chloro-8-[(5-chloro-6-fluoro-1H-indazol-4-yl) oxy]-4-(4-prop-2-enoylpiperazin-1-yl) pyrido[3,4-d]pyrimidin-2-yl]oxypropanal (37 mg, 0.066 mmol, 59% yield) as a yellow solid. LC/MS (ESI) m/z: 560.1 [M+H]⁺.

### Step 16: Preparation of (2S,4R)-1-[(2S)-2-[[2-[2-[[1-[(2R)-2-[6-chloro-8-[(5-chloro-6-fluoro-1H-indazol-4-yl)oxy]-4-(4-prop-2-enoylpiperazin-1-yl)pyrido[3,4-d]pyrimidin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

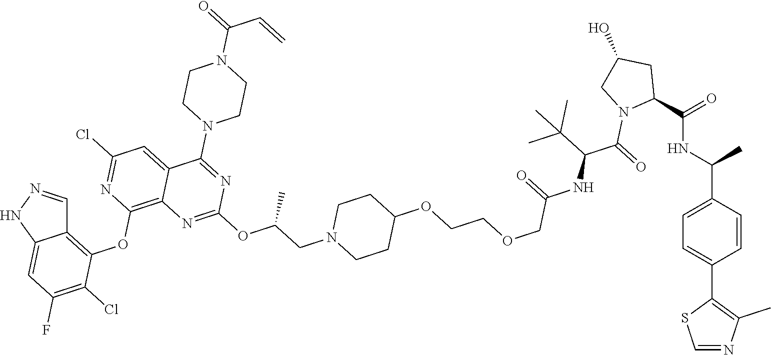 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(Oc4c(Cl)c(F)cc5[nH]ncc45)nc(Cl)cc23)CC1]

To a solution of (2S,4R)-1-[(2S)-3,3-dimethyl-2-[[2-[2-(4-piperidyloxy) ethoxy]acetyl]amino]butanoyl]-4-hydroxy-N-[(1S)-1-[4-(4-methylthiazol-5-yl)phenyl]ethyl]pyrrolidine-2-carboxamide (50 mg, 0.075 mmol, 1.2 eq, hydrochloride) in CH₃OH (1 mL) was added NaOAc (15 mg, 187.38 umol, 3 eq), and the resulting mixture was stirred at 20° C. for 20 minutes. A solution of (2R)-2-[6-chloro-8-[(5-chloro-6-fluoro-1H-indazol-4-yl)oxy]-4-(4-prop-2-enoylpiperazin-1-yl) pyrido[3,4-d]pyrimidin-2-yl]oxypropanal (35 mg, 0.062 mmol, 1 eq) in CH₂Cl₂ (1 mL) was then added, and the resulting mixture was cooled to 0° C. NaBH₄CN (8 mg, 0.12 mmol, 2 eq) was added, and the reaction mixture was stirred at 0-15° C. for 48 hours. Water (5 mL) was added, and the resulting mixture was extracted with CH₂Cl₂ (5×3 mL). The combined organic extracts were dried over anhydrous Na₂SO₄, filtered, and concentrated under reduced pressure. The resulting residue was purified by SiO₂ prep-TLC (CH₂Cl₂:CH₃OH=9:1) and freeze dried to afford (2S,4R)-1-[(2S)-2-[[2-[2-[[1-[(2R)-2-[6-chloro-8-[(5-chloro-6-fluoro-1H-indazol-4-yl)oxy]-4-(4-prop-2-enoylpiperazin-1-yl)pyrido[3,4-d]pyrimidin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide (10.3 mg, 0.008 mmol, 31% yield, 90% purity) as a white solid. LC/MS (ESI) m/z: 1175.3 [M+H]⁺. 1H-NMR (400 MHz, DMSO-d6) δ 13.62-13.53 (m, 1H), 8.98 (s, 1H), 8.45-8.37 (m, 1H), 7.77 (s, 1H), 7.70-7.57 (m, 2H), 7.44-7.40 (m, 2H), 7.35 (br d, J=8.4 Hz, 2H), 6.86-6.76 (m, 1H), 6.17 (dd, J=2.3, 17.0 Hz, 1H), 5.77-5.70 (m, 1H), 5.41-5.20 (m, 1H), 5.16-5.09 (m, 1H), 4.94-4.83 (m, 1H), 4.57-4.49 (m, 1H), 4.46-4.38 (m, 1H), 4.31-4.24 (m, 1H), 3.94 (br s, 6H), 3.85-3.73 (m, 4H), 3.62-3.54 (m, 4H), 3.53-3.47 (m, 2H), 2.84-2.70 (m, 2H), 2.45 (s, 4H), 2.22-2.09 (m, 3H), 2.07-1.93 (m, 3H), 1.81-1.71 (m, 3H), 1.39-1.32 (m, 5H), 1.30-1.25 (m, 3H), 0.91 (s, 9H).

### Exemplary Synthesis of (2S,4R)-1-[(2S)-2-[[2-[2-[[1-[(2R)-2-[[4-[(3S)-3-(cyanomethyl)-4-prop-2-enoyl-piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl4-[(3S)-4-benzyloxycarbonyl-3-(cyanomethyl)piperazin-1-yl]-2-[(1R)-2,2-dimethoxy-1-methyl-ethoxy]-6,8-dihydro-5H-pyrido[3,4-d]pyrimidine-7-carboxylate

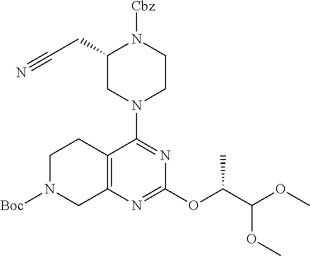 [SMILES: COC(OC)[C@@H](C)Oc1nc2c(c(N3CCN(C(=O)OCc4ccccc4)[C@@H](CC#N)C3)n1)CCN(C(=O)OC(C)(C)C)C2]

To a solution of tert-butyl 4-[(3S)-4-benzyloxycarbonyl-3-(cyanomethyl)piperazin-1-yl]-2-chloro-6,8-dihydro-5H-pyrido[3,4-d]pyrimidine-7-carboxylate (2.50 g, 4.74 mmol, 1.00 eq) and (2R)-1,1-dimethoxypropan-2-ol (1.70 g, 14.23 mmol, 3.00 eq) in dioxane (15 mL) were added C S2C03 (4.80 g, 14.23 mmol, 3.00 eq) and [2-(2-aminophenyl)phenyl]-methylsulfonyloxy-palladium; dicyclohexyl-[2-(2,6-diisopropoxyphenyl)phenyl]phosphane (278 mg, 0.33 mmol, 0.07 eq), and the reaction mixture was stirred at 90° C. for 6 hours. The solution was concentrated, and the resulting material was purified by column chromatography on SiO₂ (10-100% EtOAc in petroleum ether) to afford tert-butyl 4-[(3S)-4-benzyloxycarbonyl-3-(cyanomethyl)piperazin-1-yl]-2-[(1R)-2,2-dimethoxy-1-methyl-ethoxy]-6,8-dihydro-5H-pyrido[3,4-d]pyrimidine-7-carboxylate (1.00 g, 1.64 mmol, 35% yield) as a yellow solid. LC/MS (ESI) m/z: 611.2 [M+H]⁺.

### Step 2: Preparation of benzyl(2S)-2-(cyanomethyl)-4-[2-[(1R)-2,2-dimethoxy-1-methyl-ethoxy]-5,6,7,8-tetrahydropyrido[3,4-d]pyrimidin-4-yl]piperazine-1-carboxylate

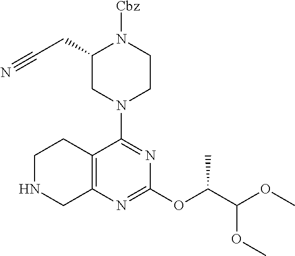 [SMILES: COC(OC)[C@@H](C)Oc1nc2c(c(N3CCN(C(=O)OCc4ccccc4)[C@@H](CC#N)C3)n1)CCNC2]

To a solution of tert-butyl 4-[(3S)-4-benzyloxycarbonyl-3-(cyanomethyl)piperazin-1-yl]-2-[(1R)-2,2-dimethoxy-1-methyl-ethoxy]-6,8-dihydro-5H-pyrido[3,4-d]pyrimidine-7-carboxylate (1.00 g, 1.64 mmol, 1.00 eq) in CH₂Cl₂ (10 mL) was added TFA (1.50 g, 13.51 mmol, 1 mL, 8.25 eq), and the reaction mixture was stirred at 25° C. for 2 hours. The mixture was neutralized (pH=7) by addition of NaHCO₃, then extracted with EtOAc (2×100 mL). The combined organic extracts were concentrated under reduced pressure to afford benzyl (2S)-2-(cyanomethyl)-4-[2-[(1R)-2,2-dimethoxy-1-methyl-ethoxy]-5,6,7,8-tetrahydropyrido[3,4-d]pyrimidin-4-yl]piperazine-1-carboxylate (800 mg, 1.57 mmol, 96% yield) as a yellow solid. LC/MS (ESI) m/z: 511.5 [M+H]⁺.

### Step 3: Preparation of benzyl(2S)-2-(cyanomethyl)-4-[2-[(1R)-2,2-dimethoxy-1-methyl-ethoxy]-7-[3-(methoxymethoxy)-1-naphthyl]-6,8-dihydro-5H-pyrido[3,4-d]pyrimidin-4-yl]piperazine-1-carboxylate

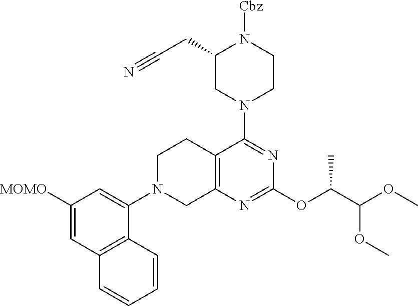

To a solution of benzyl (2S)-2-(cyanomethyl)-4-[2-[(1R)-2,2-dimethoxy-1-methyl-ethoxy]-5,6,7,8-tetrahydropyrido[3,4-d]pyrimidin-4-yl]piperazine-1-carboxylate (800 mg, 1.57 mmol, 1.00 eq) and 1-bromo-3-(methoxymethoxy)naphthalene (465 mg, 1.72 mmol, 1.10 eq) in dioxane (10 mL) were added [2-(2-aminophenyl)phenyl]-methylsulfonyloxy-palladium; dicyclohexyl-[2-(2,6-diisopropoxyphenyl)phenyl]phosphane (90 mg, 0.11 mmol, 0.07 eq), Cs₂CO₃ (1.50 g, 4.70 mmol, 3.00 eq), and Pd(OAc)₂ (20 mg, 0.08 mmol, 0.05 eq), and the reaction mixture was stirred at 90° C. for 6 hours under N₂ atmosphere. The mixture was concentrated, and the resulting material was purified by column chromatography on SiO₂ (10-100% EtOAc in petroleum ether) to afford benzyl (2S)-2-(cyanomethyl)-4-[2-[(1R)-2,2-dimethoxy-1-methyl-ethoxy]-7-[3-(methoxymethoxy)-1-naphthyl]-6,8-dihydro-5H-pyrido[3,4-d]pyrimidin-4-yl]piperazine-1-carboxylate (480 mg, 0.69 mmol, 44% yield) as a yellow solid. LC/MS (ESI) m/z: 697.2 [M+H]⁺.

### Step 4: Preparation of 2-[(2S)-4-[2-[(1R)-2,2-dimethoxy-1-methyl-ethoxy]-7-[3-(methoxymethoxy)-1-naphthyl]-6,8-dihydro-5H-pyrido[3,4-d]pyrimidin-4-yl]piperazin-2-yl]acetonitrile

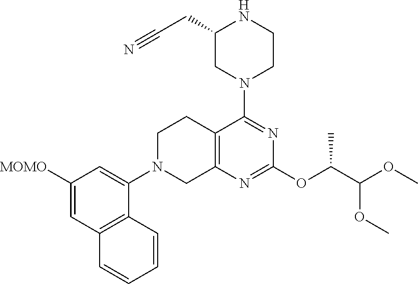

To a solution of benzyl (2S)-2-(cyanomethyl)-4-[2-[(1R)-2,2-dimethoxy-1-methyl-ethoxy]-7-[3-(methoxymethoxy)-1-naphthyl]-6,8-dihydro-5H-pyrido[3,4-d]pyrimidin-4-yl]piperazine-1-carboxylate (460 mg, 0.66 mmol, 1.00 eq) in CH³OH (15 mL) were added Pd/C carbon (100 mg) and NH₄OH (0.3 mL, 37% purity), and the reaction mixture was stirred at 25° C. for 1 hours under H₂ atmosphere. The solution was concentrated to afford 2-[(2S)-4-[2-[(1R)-2,2-dimethoxy-1-methyl-ethoxy]-7-[3-(methoxymethoxy)-1-naphthyl]-6,8-dihydro-5H-pyrido[3,4-d]pyrimidin-4-yl]piperazin-2-yl]acetonitrile (360 mg, 0.64 mmol, 96% yield) as a yellow solid. LC/MS (ESI) m/z: 563.4 [M+H]⁺.

### Step 5: Preparation of 2-[(2S)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazin-2-yl]acetonitrile

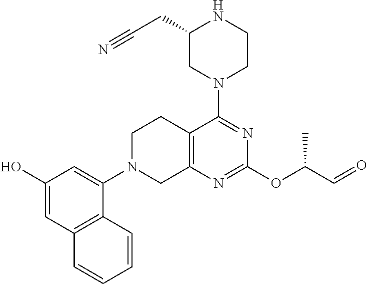 [SMILES: C[C@H](C=O)Oc1nc2c(c(N3CCN[C@@H](CC#N)C3)n1)CCN(c1cc(O)cc3ccccc13)C2]

To a solution of 2-[(2S)-4-[2-[(1R)-2,2-dimethoxy-1-methyl-ethoxy]-7-[3-(methoxymethoxy)-1-naphthyl]-6,8-dihydro-5H-pyrido[3,4-d]pyrimidin-4-yl]piperazin-2-yl]acetonitrile (210 mg, 0.37 mmol, 1.00 eq) in CH₂Cl₂ (3 mL) was added HCl (4N in dioxane, 1 mL), and the reaction mixture was stirred at 25° C. for 30 minutes. The solution was concentrated under reduced pressure, and basified (pH=8) by addition of NaHCO₃. The resulting basic mixture was extracted with EtOAc (2×50 mL), and the combined organic extracts were concentrated to afford 2-[(2S)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazin-2-yl]acetonitrile (170 mg, 0.36 mmol, 96% yield) as a yellow solid.

### Step 6: Preparation of tert-butyl(2S)-2-(cyanomethyl)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazine-1-carboxylate

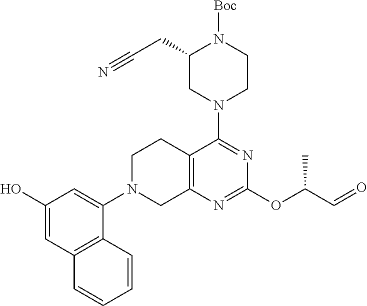 [SMILES: C[C@H](C=O)Oc1nc2c(c(N3CCN(C(=O)OC(C)(C)C)[C@@H](CC#N)C3)n1)CCN(c1cc(O)cc3ccccc13)C2]

To a solution of 2-[(2S)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazin-2-yl]acetonitrile (170 mg, 0.36 mmol, 1.00 eq) in THF (3 mL) and H₂O (1 mL) were added di-tert-butyldicarbonate (88 mg, 0.36 mmol, 1.10 eq) and NaHCO₃ (90 mg, 1.08 mmol, 3.00 eq), and the reaction mixture was stirred at 25° C. for 3 hours. The solution was concentrated, and the remaining material was purified by thin layer chromatography (CH₂Cl₂/CH₃OH=10/1) to afford tert-butyl (2S)-2-(cyanomethyl)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazine-1-carboxylate (90 mg, 0.16 mmol, 44% yield) as a yellow solid. LC/MS (ESI) m/z: 573.3 [M+H]⁺.

### Step 7: Preparation of tert-butyl(2S)-2-(cyanomethyl)-4-[2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)-6,8-dihydro-5H-pyrido[3,4-d]pyrimidin-4-yl]piperazine-1-carboxylate

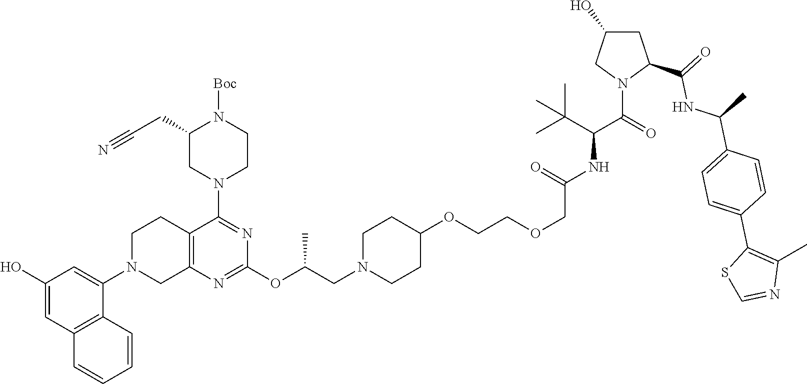 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc4c(c(N5CCN(C(=O)OC(C)(C)C)[C@@H](CC#N)C5)n3)CCN(c3cc(O)cc5ccccc35)C4)CC2)C(C)(C)C)cc1]

To a solution of tert-butyl (2S)-2-(cyanomethyl)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazine-1-carboxylate (45 mg, 0.08 mmol, 1.05 eq) and (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (50 mg, 0.07 mmol, 1.00 eq, hydrochloride salt) in CH₂Cl₂ (1 mL) and CH₃OH (1 mL) were added NaOAc (20 mg, 0.23 mmol, 3.00 eq), borane; 2-methylpyridine (40 mg, 0.38 mmol, 5.00 eq), and acetic acid (15 mg, 0.23 mmol, 3.00 eq), and the reaction mixture was stirred at 40° C. for 12 hours. The solution was concentrated under reduced pressure, and the remaining material was purified by thin layer chromatography (CH₂Cl₂/CH₃OH=10/1) to afford tert-butyl (2S)-2-(cyanomethyl)-4-[2-[(1R)-2-[4-[2-[2-[[(1S)- 1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)-6,8-dihydro-5H-pyrido[3,4-d]pyrimidin-4-yl]piperazine-1-carboxylate (40 mg, 0.03 mmol, 45% yield) as a yellow solid. LC/MS (ESI) m/z: 1186.5 [M+H]⁺.

### Step 8: Preparation of (2S,4R)-1-[(2S)-2-[[2-[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

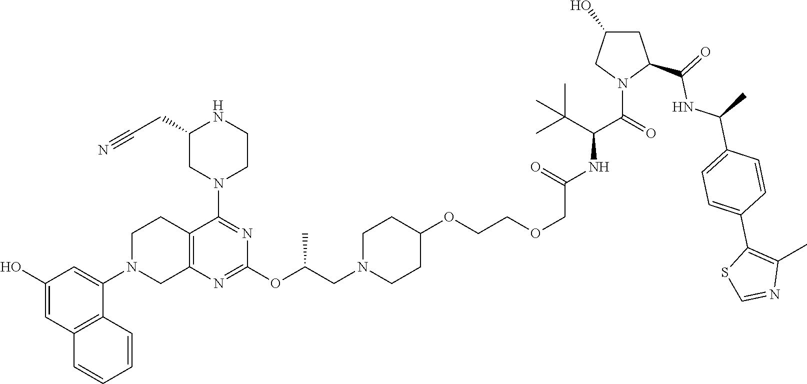 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc4c(c(N5CCN[C@@H](CC#N)C5)n3)CCN(c3cc(O)cc5ccccc35)C4)CC2)C(C)(C)C)cc1]

To a solution of tert-butyl (2S)-2-(cyanomethyl)-4-[2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)-6,8-dihydro-5H-pyrido[3,4-d]pyrimidin-4-yl]piperazine-1-carboxylate (30 mg, 0.03 mmol, 1.00 eq) in CH₂Cl₂ (0.5 mL) was added HCl (4N in dioxane, 0.5 mL), and the reaction mixture was stirred at 25° C. for 0.5 hours. The solution was concentrated under reduced pressure and the remaining material was taken up in EtOAc and CH₃CN and the solutions concentrated in vacuum multiple times to afford (2S,4R)-1-[(2S)-2-[[2-[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (28 mg, 0.02 mmol, 96% yield, two hydrochloride salts) as a yellow solid. LC/MS (ESI) m/z: 1086.2 [M+H]⁺.

### Step 9: Preparation of (2S,4R)-1-[(2S)-2-[[2-[2-[[1-[(2R)-2-[[4-[(3S)-3-(cyanomethyl)-4-prop-2-enoyl-piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

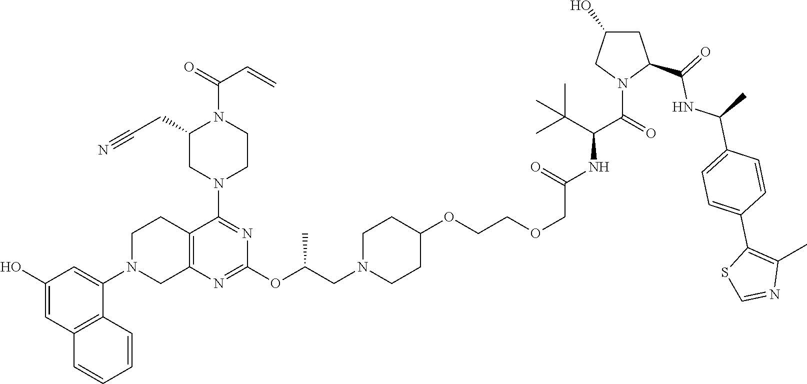 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N]

To a solution of (2S,4R)-1-[(2S)-2-[[2-[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (28 mg, 0.02 mmol, 1.00 eq, two hydrochloride salts) in CH₂Cl₂ (15 mL) were added 2,6-dimethylpyridine (20 mg, 0.19 mmol, 8.00 eq) and prop-2-enoyl chloride (2 mg, 0.02 mmol, 1.00 eq), and the reaction mixture was stirred at −75° C. for 0.5 hours. The solution was concentrated under reduced pressure, and the remaining material was purified by prep-HPLC [30-60% CH₃CN in water (10 mM NH₄HCO₃)] to afford (2S,4R)-1-[(2S)-2-[[2-[2-[[1-[(2R)-2-[[4-[(3S)-3-(cyanomethyl)-4-prop-2-enoyl- piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (4.0 mg, 14% yield, 97% purity) as a white solid. LC/MS (ESI) m/z: 1140.4 [M+H]⁺. ¹H-NMR (400 MHz, CD₃OD) δ 8.85 (s, 1H), 8.06 (d, J=8.8 Hz, 1H), 7.62 (d, J=8.0 Hz, 1H), 7.44-7.32 (m, 5H), 7.29-7.19 (m, 1H), 6.99-6.66 (m, 3H), 6.29 (d, J=16.8 Hz, 1H), 5.83 (d, J=11.2 Hz, 1H), 5.42 (d, J=2.8 Hz, 1H), 5.18-5.03 (m, 1H), 5.01-4.95 (m, 1H), 4.82-4.48 (m, 4H), 4.47-4.29 (m, 1H), 4.21-3.97 (m, 6H), 3.89-3.35 (m, 9H), 3.12-2.81 (m, 6H), 2.73 (dd, J=7.6, 13.2 Hz, 1H), 2.52 (dd, J=2.8, 13.6 Hz, 1H), 2.48-2.40 (m, 3H), 2.39-2.25 (m, 2H), 2.19 (dd, J=7.6, 12.8 Hz, 1H), 2.03-1.79 (m, 3H), 1.64-1.53 (m, 2H), 1.47 (d, J=7.2 Hz, 3H), 1.33 (d, J=6.0 Hz, 3H), 1.02 (s, 9H).

The following compounds can be prepared in an analogous manner to 2S,4R)-1-[(2S)-2-[[2-[2-[[1-[(2R)-2-[[4-[(3S)-3-(cyanomethyl)-4-prop-2-enoyl-piperazin-1-yl]-7-(3-hydroxy-1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. Exemplary Compound (2S,4R)-1-[(2S)-2-[[2-[2-[[1-[(2R)-2-[[4-[(3S)-3-(cyanomethyl)-4-prop-2-enoyl-piperazin-1-yl]-7-(1-naphthyl)-6,8-dihydro-5H-pyrido[3,4-d]pyrimidin-2-yl]oxy]propyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

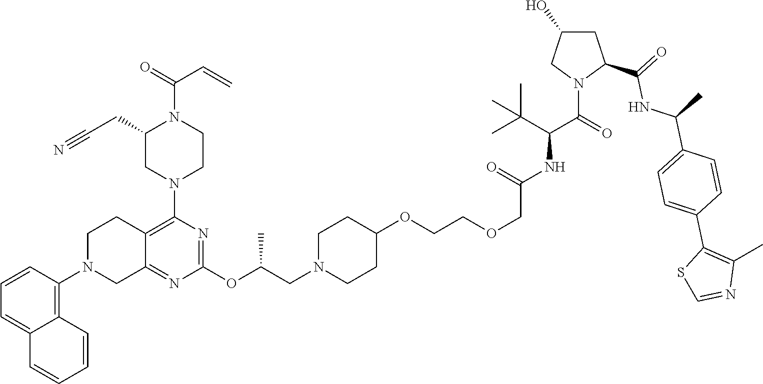 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cccc4ccccc24)C3)C[C@@H]1CC#N]

(formic acid salt, white solid). LC/MS (ESI) m/z: 1124.4 [M+H]⁺. ¹H-NMR (400 MHz, CD₃OD) δ 8.88 (s, 1H), 8.54 (s, 1H), 8.28-8.21 (m, 1H), 7.91-7.84 (m, 1H), 7.62 (d, J=8.0 Hz, 1H), 7.54-7.47 (m, 2H), 7.46-7.35 (m, 5H), 7.21 (d, J=7.2 Hz, 1H), 6.81 (s, 1H), 6.31 (d, J=16.4 Hz, 1H), 5.85 (d, J=10.0 Hz, 1H), 5.50 (s, 1H), 5.19-4.93 (m, 2H), 4.86-4.77 (m, 1H), 4.65-4.54 (m, 2H), 4.48-4.35 (m, 1H), 4.29-4.01 (m, 7H), 3.88-3.80 (m, 1H), 3.79-3.59 (m, 6H), 3.58-3.36 (m, 3H), 3.29-2.59 (m, 12H), 2.51-2.43 (m, 3H), 2.26-2.17 (m, 1H), 2.06-1.91 (m, 3H), 1.74 (s, 2H), 1.49 (d, J=7.2 Hz, 3H), 1.39 (d, J=6 Hz, 3H), 1.05 (s, 9H).

### 2. Exemplary Compound (2S,4R)-1-[(2S)-2-[[2-[2-[[1-[2-[[4-[(3S)-3-(cyanomethyl)-4-prop- 2-enoyl-piperazin-1-yl]-7-(8-methyl-1-naphthyl)-6,8-dihydro-5H-pyrido[3,4-d]pyrimidin-2-yl]oxy]ethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

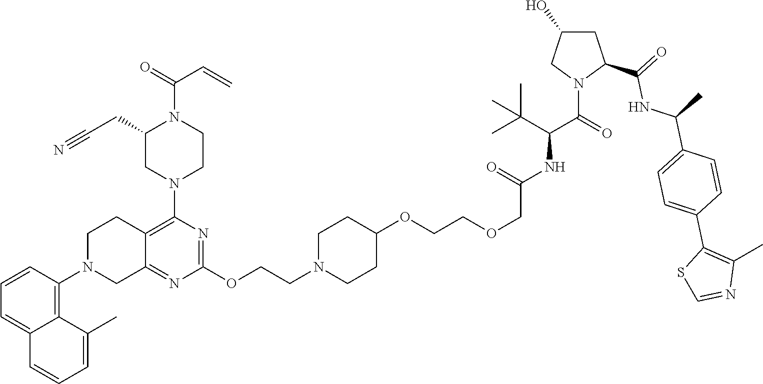 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cccc4cccc(C)c24)C3)C[C@@H]1CC#N]

(formic acid salt, white solid). LC/MS (ESI) m/z: 1123.6[M+H]⁺. ¹H-NMR (400 MHz, CD₃OD) δ 8.94-8.76 (m, 1H), 8.52 (d, J=1.6 Hz, 1H), 7.66 (dd, J=7.6, 17.2 Hz, 2H), 7.50-7.15 (m, 8H), 6.81 (d, J=2.4 Hz, 1H), 6.29 (d, J=16.0 Hz, 1H), 5.83 (d, J=11.2 Hz, 1H), 5.16-4.94 (m, 2H), 4.79 (d, J=7.6 Hz, 1H), 4.65-4.64 (m, 1H), 4.64-4.47 (m, 3H), 4.49-4.27 (m, 1H), 4.26-3.98 (m, 5H), 3.90-3.41 (m, 11H), 3.28-2.94 (m, 9H), 2.90 (s, 4H), 2.84-2.59 (m, 3H), 2.46 (s, 3H), 2.38-2.14 (m, 1H), 2.07-1.69 (m, 5H), 1.61-1.39 (m, 3H), 1.03 (s, 9H).

### Exemplary Synthesis of (2S,4R)-1-[(2S)-2-[[2-[[1-[(2R)-2-[[4-[(3S)-3-(cyanomethyl)-4-prop-2-enoyl-piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl (2S)-2-(cyanomethyl)-4-[2-[(1R)-2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]-1-methyl-ethoxy]-7-(3-hydroxy-1-naphthyl)-6,8-dihydro-5H-pyrido[3,4-d]pyrimidin-4-yl]piperazine-1-carboxylate

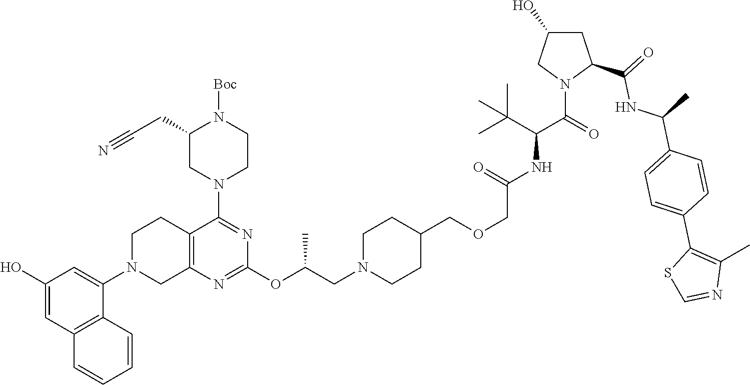 [SMILES: Cc1ncsc1-c1ccc([C@H](C)NC(=O)[C@@H]2C[C@@H](O)CN2C(=O)[C@@H](NC(=O)COCC2CCN(C[C@@H](C)Oc3nc4c(c(N5CCN(C(=O)OC(C)(C)C)[C@@H](CC#N)C5)n3)CCN(c3cc(O)cc5ccccc35)C4)CC2)C(C)(C)C)cc1]

To a solution of (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (45 mg, 0.07 mmol, 1.01 eq, hydrogen chloride) and tert-butyl (2S)-2-(cyanomethyl)-4-[7-(3-hydroxy-1-naphthyl)-2-[(1R)-1-methyl-2-oxo-ethoxy]-6,8-dihydro-5H-pyrido[3,4-d]pyrimidin-4-yl]piperazine-1-carboxylate (40 mg, 0.07 mmol, 1.00 eq) in CH₂Cl₂ (0.7 mL) and CH₃OH (0.7 mL) were added NaOAc (60 mg, 0.70 mmol, 10.00 eq) and acetic acid (cat.) at 25° C., and the resulting mixture was stirred at 25° C. for 2 hours. 2-Methylpyridine borane (40 mg, 0.37 mmol, 5.35 eq) was added at 0° C., and the reaction mixture was stirred at 40° C. for 8 hours. The mixture was filtered, and the filtrate was concentrated under reduced pressure. The resulting residue was purified by prep-thin layer chromatography (CH₂Cl₂:CH₃OH=10:1) to afford tert-butyl (2S)-2-(cyanomethyl)-4-[2-[(1R)-2-[4-[[2-[[(1S)-1- [(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]-1-methyl-ethoxy]-7-(3-hydroxy-1-naphthyl)-6,8-dihydro-5H-pyrido[3,4-d]pyrimidin-4-yl]piperazine-1-carboxylate (50 mg, 0.04 mmol, 61% yield) as a brown solid. LC/MS (ESI) m/z: 1156.9 [M+H]⁺.

### Step 2: Preparation of (2S,4R)-1-[(2S)-2-[[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

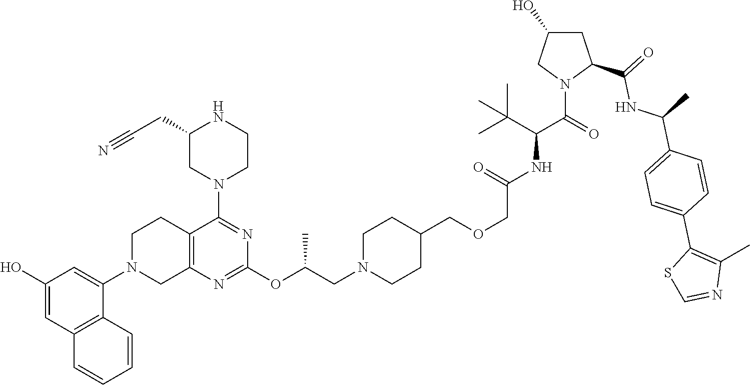 [SMILES: Cc1ncsc1-c1ccc([C@H](C)NC(=O)[C@@H]2C[C@@H](O)CN2C(=O)[C@@H](NC(=O)COCC2CCN(C[C@@H](C)Oc3nc4c(c(N5CCN[C@@H](CC#N)C5)n3)CCN(c3cc(O)cc5ccccc35)C4)CC2)C(C)(C)C)cc1]

To a solution of tert-butyl (2S)-2-(cyanomethyl)-4-[2-[(1R)-2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]-1-methyl-ethoxy]-7-(3-hydroxy-1-naphthyl)-6,8-dihydro-5H-pyrido[3,4-d]pyrimidin-4-yl]piperazine-1-carboxylate (50 mg, 0.04 mmol, 1.00 eq) in CH₂Cl₂ (2 mL) was added TFA (13.51 mmol, 1 mL, 312.38 eq), and the reaction mixture was stirred at 25° C. for 10 minutes. The mixture was concentrated under reduced pressure to give (2S,4R)-1-[(2S)-2-[[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (50 mg, 0.04 mmol, 90% yield, 2 trifluoroacetic acid salts) as a red oil. LC/MS (ESI) m/z: 1056.7 [M+H]⁺.

### Step 3: Preparation of (2S,4R)-1-[(2S)-2-[[2-[[1-[(2R)-2-[[4-[(3S)-3-(cyanomethyl)-4-prop- 2-enoyl-piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

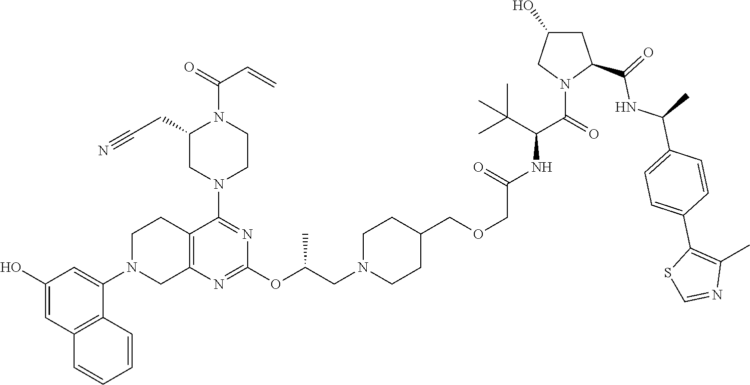 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N]

To a solution of (2S,4R)-1-[(2S)-2-[[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (50 mg, 0.04 mmol, 1.00 eq, 2 trifluoroacetic acid salt) in CH₂Cl₂ (4 mL) at −78° C. were added 2,6-lutidine (0.40 mmol, 10.00 eq) and prop-2-enoyl chloride (4 mg, 0.04 mmol, 1.00 eq), and the reaction mixture was stirred at −78° C. for 30 minutes. Water was then added at −78° C., and the resulting mixture was concentrated under reduced pressure. The resulting residue was purified by semi-preparative reverse phase HPLC (35-65% CH₃CN in water (0.01M NH₄HCO₃)), to afford (2S,4R)-1-[(2S)-2-[[2-[[1-[(2R)-2-[[4-[(3S)-3-(cyanomethyl)-4-prop-2-enoyl-piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (7.5 mg, 0.006 mmol, 16% yield, 96% purity) as a white solid after lyophilization. LC/MS (ESI) m/z: 1110.6 [M+H]⁺. ¹H-NMR (400 MHz, CD₃OD) δ 8.86 (s, 1H), 8.06 (d, J=8.4 Hz, 1H), 7.61 (d, J=8.0 Hz, 1H), 7.46-7.31 (m, 5H), 7.29-7.20 (m, 1H), 6.94-6.71 (m, 3H), 6.29 (d, J=16.4 Hz, 1H), 5.83 (d, J=10.0 Hz, 1H), 5.44 (s, 1H), 5.16-4.93 (m, 2H), 4.72-4.32 (m, 5H), 4.25-4.02 (m, 5H), 4.01-3.90 (m, 2H), 3.86-3.79 (m, 1H), 3.77-3.69 (m, 1H), 3.63 (s, 1H), 3.38 (d, J=6.0 Hz, 3H), 3.13-2.85 (m, 6H), 2.75 (dd, J=8.4, 13.6 Hz, 1H), 2.59-2.50 (m, 1H), 2.46 (s, 3H), 2.25-2.09 (m, 3H), 1.96 (dd, J=4.0, 9.2 Hz, 1H), 1.81-1.59 (m, 3H), 1.58-1.43 (m, 3H), 1.41-1.10 (m, 6H), 1.02 (s, 9H).

### Exemplary Synthesis of (2S,4R)-1-[(2S)-2-[[2-[[1-[(2R)-2-[[4-[(3S)-3-(cyanomethyl)-4-(2-fluoroprop-2-enoyl)piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

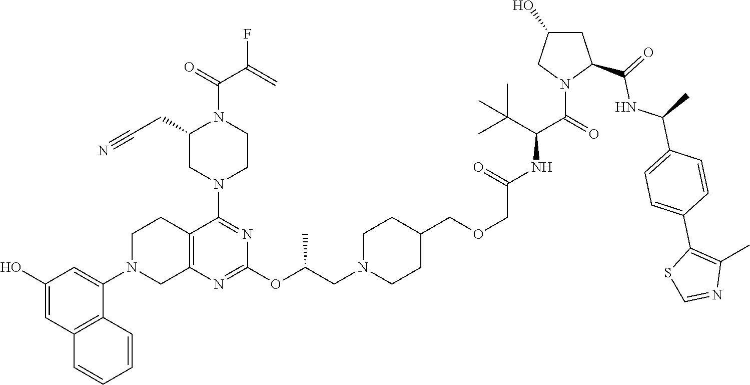 [SMILES: C=C(F)C(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N]

To a solution of (2S,4R)-1-[(2S)-2-[[2-[[1-[(2R)-2-[[4-[(3S)-3-(cyanomethyl)piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (100 mg, 0.09 mmol, 1.00 eq, hydrochloride salt) and 2-fluoroprop-2-enoic acid (8 mg, 0.09 mmol, 1.00 eq) in DMF (2 mL) were added HATU (55 mg, 0.14 mmol, 1.50 eq) and diisopropylethylamine (25 mg, 0.18 mmol, 2.00 eq), and the reaction mixture was stirred at 25° C. for 1 hour. The solution was concentrated under reduced pressure, and the remaining material was purified by prep-HPLC (25-55% CH₃CN in water (0.225% formic acid)) to afford (2S,4R)-1-[(2S)-2-[[2-[[1-[(2R)-2-[[4-[(3S)-3-(cyanomethyl)-4-(2-fluoroprop-2-enoyl)piperazin-1-yl]-7-(3-hydroxy-1-naphthyl)-6,8-dihydro-5H-pyrido[3,4-d]pyrimidin-2-yl]oxy]propyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (5.6 mg, 5% yield, 99% purity) as a white solid. LC/MS (ESI) m/z: 1129.6 [M+H]⁺. ¹H-NMR (400 MHz, CD₃OD) δ 8.79-8.74 (m, 1H), 7.97 (d, J=9.2 Hz, 1H), 7.52 (d, J=7.6 Hz, 1H), 7.36-7.22 (m, 7H), 7.16 (s, 1H), 6.77 (d, J=1.6 Hz, 1H), 6.69 (d, J=2.4 Hz, 1H), 5.42-5.27 (m, 1H), 5.26-5.05 (m, 2H), 4.92-4.88 (m, 1H), 4.57 (s, 1H), 4.46 (s, 2H), 4.38-4.22 (m, 1H), 4.16-4.00 (m, 5H), 3.93-3.79 (m, 3H), 3.77-3.38 (m, 4H), 3.30 (d, J=6.4 Hz, 4H), 3.09-3.01 (m, 4H), 3.00-2.89 (m, 2H), 2.67-2.51 (m, 1H), 2.36 (s, 3H), 2.32-2.00 (m, 4H), 1.84 (dd, J=4.0, 12.8 Hz, 1H), 1.76-1.64 (m, 3H), 1.37 (d, J=7.2 Hz, 3H), 1.26 (d, J=6.4 Hz, 5H), 0.93-0.91 (m, 9H).

### Exemplary Synthesis of (2S,4R)-1-[(2S)-2-[[2-[[1-[3-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of ethyl 2-(4-piperidyloxy)acetate

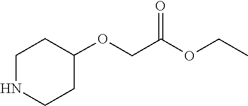

A solution of tert-butyl 4-(2-ethoxy-2-oxo-ethoxy)piperidine-1-carboxylate (2.7 g, 9.40 mmol, 1 eq) and HCl (4M in dioxane, 15.00 mL, 6.39 eq) in CH₂Cl₂ (30 mL) was stirred at 20° C. for 2 hours. The reaction was concentrated under reduced pressure to afford ethyl 2-(4-piperidyloxy)acetate (2.1 g, 9.39 mmol, 99.9% yield, hydrochloric acid) as an off-white solid.

### Step 2: Preparation of ethyl 2-[[1-(3-benzyloxypropyl)-4-piperidyl]oxy]acetate

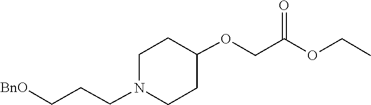 [SMILES: CCOC(=O)COC1CCN(CCCOCc2ccccc2)CC1]

To a solution of 3-benzyloxypropyl 4-methylbenzenesulfonate (1.19 g, 3.70 mmol, 1.2 eq) and ethyl 2-(4-piperidyloxy)acetate (690 mg, 3.08 mmol, 1 eq, hydrochloric acid) in DMF (3 mL) was added K₂CO₃ (853 mg, 6.17 mmol, 2 eq), and the reaction mixture was stirred at 50° C. for 12 hours. Water (30 mL) was then added, and the resulting mixture was extracted with EtOAc (2×20 mL). The combined organic extracts were washed with brine (30 mL), dried over Na₂SO₄, filtered, and concentrated under reduced pressure. The resulting residue was purified by prep-TLC on SiO₂ (CH₂Cl₂:CH₃OH=10:1) to afford ethyl 2-[[1-(3-benzyloxypropyl)-4-piperidyl]oxy]acetate (570 mg, 1.70 mmol, 55.09% yield) as a colorless oil. ¹H-NMR (400 MHz, CDCl₃) δ 7.27 (s, 5H), 4.44 (s, 2H), 4.22-4.12 (m, 2H), 4.06-3.98 (m, 2H), 3.51-3.28 (m, 3H), 2.77-2.66 (m, 2H), 2.43-2.29 (m, 2H), 2.07 (t, J=9.7 Hz, 2H), 1.93-1.82 (m, 2H), 1.80-1.68 (m, 2H), 1.61 (dtd, J=3.6, 9.3, 12.8 Hz, 2H), 1.23 (t, J=7.1 Hz, 3H).

### Step 3: Preparation of methyl 2-[[1-(3-hydroxypropyl)-4-piperidyl]oxy]acetate

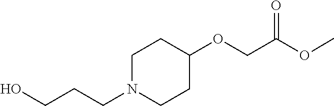 [SMILES: COC(=O)COC1CCN(CCCO)CC1]

To a solution of ethyl 2-[[1-(3-benzyloxypropyl)-4-piperidyl]oxy]acetate (570 mg, 1.70 mmol, 1 eq) in CH₃OH (10 mL) was added Pd/C (50 mg, 5%) under N₂ atmosphere, and the resulting suspension was degassed under vacuum and purged with H₂ several times. The resulting mixture was then stirred under H₂ (50 psi) at 50° C. for 12 hours. The reaction was filtered, and the filtrate was concentrated under reduced pressure to afford crude methyl 2-[[1-(3-hydroxypropyl)-4-piperidyl]oxy]acetate (400 mg) as a colorless oil.

### Step 4: Preparation of tert-butyl 4-[7-bromo-6-chloro-8-fluoro-2-[3-[4-(2-methoxy-2-oxo-ethoxy)-1-piperidyl]propoxy]quinazolin-4-yl]piperazine-1-carboxylate

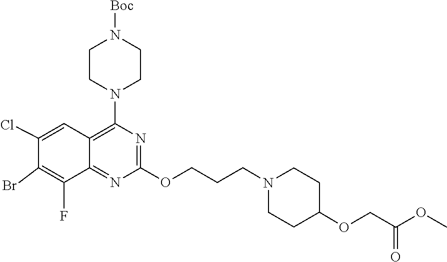 [SMILES: COC(=O)COC1CCN(CCCOc2nc(N3CCN(C(=O)OC(C)(C)C)CC3)c3cc(Cl)c(Br)c(F)c3n2)CC1]

A mixture of methyl 2-[[1-(3-hydroxypropyl)-4-piperidyl]oxy]acetate (217 mg, 0.94 mmol, 1.5 eq), tert-butyl 4-(7-bromo-2,6-dichloro-8-fluoro-quinazolin-4-yl)piperazine-1-carboxylate (300 mg, 0.62 mmol, 1 eq), Cs₂CO₃ (407 mg, 1.25 mmol, 2 eq), and 1,4-diazabicyclo[2.2.2]octane (21 mg, 0.19 mmol, 0.02 mL, 0.3 eq) in CH₃CN (20 mL) was stirred at 50° C. for 2 hours. The mixture was filtered, and the filtrate was concentrated under reduced pressure. The resulting residue was purified by semi-preparative reverse phase HPLC (30-60% CH₃CN in water (0.225% formic acid) to afford-butyl 4-[7-bromo-6-chloro-8-fluoro-2-[3-[4-(2-methoxy-2-oxo-ethoxy)-1-piperidyl]propoxy]quinazolin-4-yl]piperazine-1-carboxylate (100 mg, 0.15 mmol, 23.7% yield) as a colorless oil. LC/MS (ESI) m/z: 676.2 [M+H]⁺.

### Step 5: Preparation of tert-butyl 4-[6-chloro-8-fluoro-7-(3-hydroxy-1-naphthyl)-2-[3-[4-(2-methoxy-2-oxo-ethoxy)-1-piperidyl]propoxy]quinazolin-4-yl]piperazine-1-carboxylate

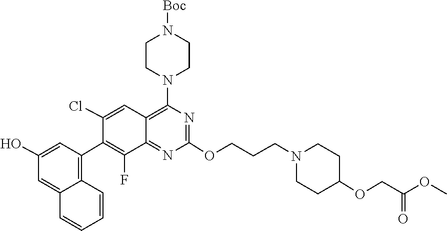 [SMILES: COC(=O)COC1CCN(CCCOc2nc(N3CCN(C(=O)OC(C)(C)C)CC3)c3cc(Cl)c(-c4cc(O)cc5ccccc45)c(F)c3n2)CC1]

To a solution of tert-butyl 4-[7-bromo-6-chloro-8-fluoro-2-[3-[4-(2-methoxy-2-oxo-ethoxy)-1-piperidyl]propoxy]quinazolin-4-yl]piperazine-1-carboxylate (100 mg, 0.15 mmol, 1 eq) and 4-(4,4,5,5-tetramethyl-1,3,2-dioxaborolan-2-yl)naphthalen-2-ol (40 mg, 0.15 mmol, 1 eq) in THF (20 mL) were added K₃PO₄ (1.5 M, 0.3 mL, 3 eq) and methanesulfonato(2-dicyclohexylphosphino-2,4,6-tri-i-propyl-1,1-biphenyl)(2-amino-1,1-biphenyl-2-yl)palladium(II) (12.54 mg, 0.02 mmol, 0.1 eq), and the reaction mixture was stirred at 50° C. for 12 hours under N₂ atmosphere. Water (30 mL) was then added, and the resulting mixture was extracted with EtOAc (2×20 mL). The combined organic extracts were washed with brine (30 mL), dried over Na₂SO₄, filtered, and concentrated under reduced pressure. The resulting residue was purified by prep-TLC on SiO₂ (CH₂Cl₂:CH₃OH=10:1) to afford tert-butyl 4-[6-chloro-8-fluoro-7-(3-hydroxy-1-naphthyl)-2-[3-[4-(2-methoxy-2-oxo-ethoxy)-1-piperidyl]propoxy]quinazolin-4-yl]piperazine-1-carboxylate (55 mg, 0.07 mmol, 45.8% yield, 91% purity) as a yellow oil. LC/MS (ESI) m/z: 738.3 [M+H]⁺.

### Step 6: Preparation of 2-[[1-[3-[4-(4-tert-butoxycarbonylpiperazin-1-yl)-6-chloro-8-fluoro-7-(3-hydroxy-1-naphthyl)quinazolin-2-yl]oxypropyl]-4-piperidyl]oxy]acetic Acid

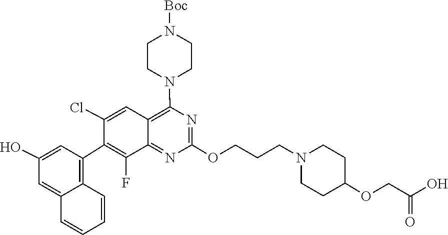 [SMILES: CC(C)(C)OC(=O)N1CCN(c2nc(OCCCN3CCC(OCC(=O)O)CC3)nc3c(F)c(-c4cc(O)cc5ccccc45)c(Cl)cc23)CC1]

A solution of tert-butyl 4-[6-chloro-8-fluoro-7-(3-hydroxy-1-naphthyl)-2-[3-[4-(2-methoxy-2-oxo-ethoxy)-1-piperidyl]propoxy]quinazolin-4-yl]piperazine-1-carboxylate (55 mg, 0.07 mmol, 1 eq) and LiOH (9 mg, 0.22 mmol, 3 eq) in water (0.5 mL), THF (0.5 mL), and CH₃OH (0.5 mL) was stirred at 20° C. for 0.5 hours. The mixture was concentrated under reduced pressure to afford crude 2-[[1-[3-[4-(4-tert-butoxycarbonylpiperazin-1-yl)-6-chloro-8-fluoro-7-(3-hydroxy-1-naphthyl)quinazolin-2-yl]oxypropyl]-4-piperidyl]oxy]acetic acid (53 mg, 0.07 mmol, 98.2% yield) as a colorless oil.

### Step 8: Preparation of tert-butyl 4-[6-chloro-8-fluoro-2-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]-1-piperidyl]propoxy]-7-(3-hydroxy-1-naphthyl)quinazolin-4-yl]piperazine-1-carboxylate

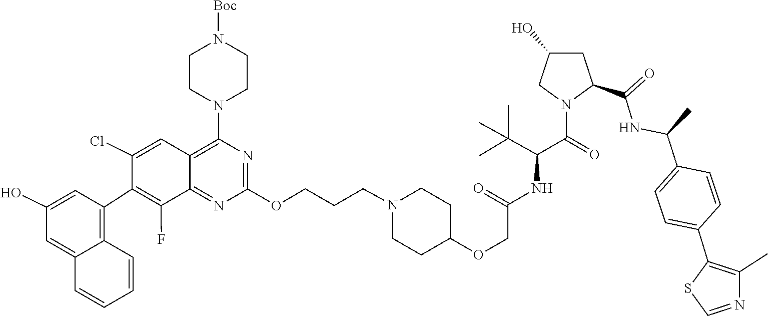 [SMILES: Cc1ncsc1-c1ccc([C@H](C)NC(=O)[C@@H]2C[C@@H](O)CN2C(=O)[C@@H](NC(=O)COC2CCN(CCCOc3nc(N4CCN(C(=O)OC(C)(C)C)CC4)c4cc(Cl)c(-c5cc(O)cc6ccccc56)c(F)c4n3)CC2)C(C)(C)C)cc1]

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (35 mg, 0.07 mmol, 1 eq, hydrochloric acid) and 2-[[1-[3-[4-(4-tert-butoxycarbonylpiperazin-1-yl)-6-chloro-8-fluoro-7-(3-hydroxy-1-naphthyl)quinazolin-2-yl]oxypropyl]-4-piperidyl]oxy]acetic acid (53 mg, 0.07 mmol, 1 eq) in DMF (2 mL) were added HATU (56 mg, 0.15 mmol, 2 eq) and diisopropylethylamine (28 mg, 0.22 mmol, 0.04 mL, 3 eq), and the reaction mixture was stirred at 25° C. for 0.5 hours. Water (30 mL) was then added, and the resulting mixture was extracted with EtOAc (2×20 mL). The combined organic extracts were washed with brine (30 mL), dried over Na₂SO₄, filtered, and concentrated under reduced pressure. The resulting residue was purified by prep-TLC on SiO₂ (CH₂Cl₂:CH₃OH=9:1) to afford tert-butyl 4-[6-chloro-8-fluoro-2-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]-1-piperidyl]propoxy]-7-(3-hydroxy-1-naphthyl)quinazolin-4-yl]piperazine-1-carboxylate (47 mg, 0.04 mmol, 55.8% yield) as a colorless oil. LC/MS (ESI) m/z: 1150.6 [M+H]⁺.

### Step 9: Preparation of (2S,4R)-1-[(2S)-2-[[2-[[1-[3-[6-chloro-8-fluoro-7-(3-hydroxy-1-naphthyl)-4-piperazin-1-yl-quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

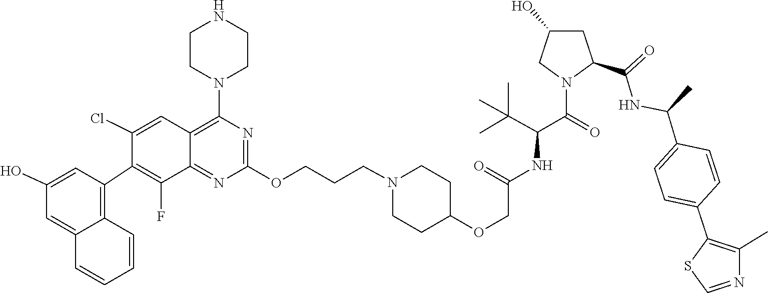 [SMILES: Cc1ncsc1-c1ccc([C@H](C)NC(=O)[C@@H]2C[C@@H](O)CN2C(=O)[C@@H](NC(=O)COC2CCN(CCCOc3nc(N4CCNCC4)c4cc(Cl)c(-c5cc(O)cc6ccccc56)c(F)c4n3)CC2)C(C)(C)C)cc1]

To a solution of tert-butyl 4-[6-chloro-8-fluoro-2-[3-[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]-1-piperidyl]propoxy]-7-(3-hydroxy-1-naphthyl)quinazolin-4-yl]piperazine-1-carboxylate (47 mg, 0.04 mmol, 1 eq) in CH₂Cl₂ (7 mL) was added TFA, and the reaction mixture was stirred at 20° C. for 0.5 hours. The mixture was concentrated under reduced pressure to give (2S,4R)-1-[(2S)-2-[[2-[[1-[3-[6-chloro-8-fluoro-7-(3-hydroxy-1-naphthyl)-4-piperazin-1-yl-quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (47 mg, 0.04 mmol, 99% yield, TFA salt) as a colorless oil. LC/MS (ESI) m/z: 1050.4 [M+H]⁺.

### Step 10: Preparation of (2S,4R)-1-[(2S)-2-[[2-[[1-[3-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

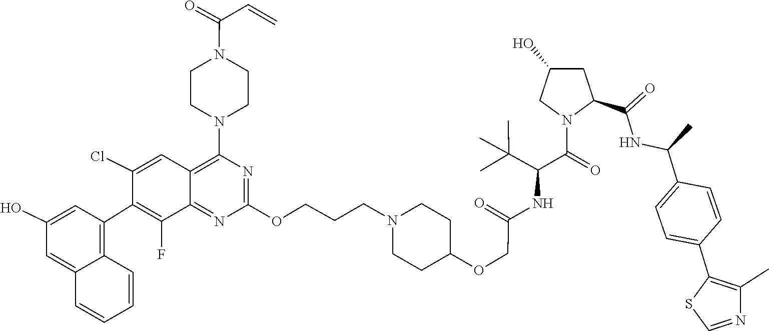 [SMILES: C=CC(=O)N1CCN(c2nc(OCCCN3CCC(OCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To a solution of (2S,4R)-1-[(2S)-2-[[2-[[1-[3-[6-chloro-8-fluoro-7-(3-hydroxy-1-naphthyl)-4-piperazin-1-yl-quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (47 mg, 0.04 mmol, 1 eq, trifluoroacetic acid) and 2,6-lutidine (130 mg, 1.21 mmol, 0.14 mL, 30 eq) in CH₂Cl₂ (3 mL) at −78° C., was added a solution of prop-2-enoyl chloride (3.29 mg, 0.04 mmol, 0.003 mL, 0.9 eq) in CH₂Cl₂ (1 mL) dropwise, and the reaction mixture was stirred at −78° C. for 30 minutes. Water (30 mL) was then added, and the resulting mixture was extracted with CH₂Cl₂ (2×20 mL). The combined organic extracts were washed with brine (30 mL), dried over Na₂SO₄, filtered, and concentrated under reduced pressure. The resulting residue was purified by semi-preparative reverse phase HPLC (20-50% CH₃CN in water (0.225% formic acid)) to afford (2S,4R)-1-[(2S)-2-[[2-[[1-[3-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (7.47 mg, 0.006 mmol, 15% yield, 94% purity, formic acid) as a white solid. LC/MS (ESI) m/z: 1150.3 [M+H]⁺. ¹H-NMR (400 MHz, DMSO-d₆) δ (s, 1H), 8.45 (d, J=7.7 Hz, 1H), 8.37 (s, 1H), 8.01 (s, 1H), 7.80 (d, J=8.3 Hz, 1H), 7.49-7.40 (m, 3H), 7.39-7.26 (m, 4H), 7.25-7.18 (m, 2H), 7.07 (d, J=2.2 Hz, 1H), 6.83 (dd, J=10.3, 16.7 Hz, 1H), 6.18 (dd, J=2.4, 16.8 Hz, 1H), 5.79-5.69 (m, 1H), 4.88 (t, J=7.2 Hz, 1H), 4.52 (d, J=9.7 Hz, 1H), 4.48-4.32 (m, 3H), 4.27 (s, 1H), 4.02-3.46 (m, 12H), 2.75-2.68 (m, 1H), 2.53-2.52 (m, 2H), 2.45 (s, 3H), 2.44-2.39 (m, 3H), 2.33 (s, 1H), 2.13-2.02 (m, 3H), 1.93-1.74 (m, 5H), 1.52-1.43 (m, 2H), 1.36 (d, J=7.1 Hz, 3H), 0.92 (s, 9H).

### Exemplary Synthesis of (2S,4R)-1-[(2S)-2-[[2-[[1-[(1R,2R)-2-[6-chloro-8-fluoro-7-(3-hydroxy-1-naphthyl)-4-(4-prop-2-enoylpiperazin-1-yl)quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide and (2S,4R)-1-[(2S)-2-[[2-[[1-[(1S,2S)-2-[6-chloro-8-fluoro-7-(3-hydroxy-1-naphthyl)-4-(4-prop-2-enoylpiperazin-1-yl)quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of benzyl 4-((2-(tert-butoxy)-2-oxoethoxy)methyl)piperidine-1-carboxylate

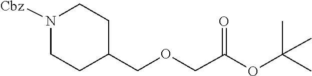 [SMILES: CC(C)(C)OC(=O)COCC1CCN(C(=O)OCc2ccccc2)CC1]

To a mixture of benzyl 4-(hydroxymethyl)piperidine-1-carboxylate (10 g, 40.1 mmol, 1 eq) in THF (50 mL) was added NaH (3.2 g, 80.2 mmol, 60%, 2 eq) slowly followed by tert-butyl 2-bromoacetate (15.7 g, 80.2 mmol, 2 eq), and the reaction mixture was stirred at 25° C. for 12 hours. The reaction mixture was quenched by addition water (10 mL), and the resulting mixture was extracted with EtOAc (3×50 mL). The combined organic extracts were washed with brine (3×50 mL), dried over anhydrous Na₂SO₄, filtered, and concentrated in vacuum. The resulting residue was purified by silica gel chromatography (10-20% EtOAc in petroleum ether) to afford benzyl 4-[(2-tert-butoxy-2-oxo-ethoxy)methyl]piperidine-1-carboxylate (3.6 g, 9.91 mmol, 25% yield) as a colorless oil. ¹H-NMR (400 MHz, CDCl₃) δ 7.42-7.30 (m, 4H), 5.14 (s, 2H), 4.19 (d, J=18.2 Hz, 2H), 3.96 (s, 2H), 3.38 (d, J=6.4 Hz, 2H), 2.81 (s, 2H), 1.79 (d, J=13.2 Hz, 3H), 1.53-1.47 (m, 9H), 1.26-1.13 (m, 2H).

### Step 2: Preparation of tert-butyl 2-(piperidin-4-ylmethoxy)acetate

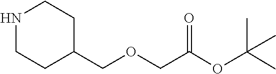 [SMILES: CC(C)(C)OC(=O)COCC1CCNCC1]

To a solution of benzyl 4-[(2-tert-butoxy-2-oxo-ethoxy)methyl]piperidine-1-carboxylate (3.1 g, 8.53 mmol, 1 eq) and NH₄OH (107 mg, 0.85 mmol, 28% purity, 0.1 eq) in CH₃OH (30 mL) was added Pd/C (300 mg, 10%) under N₂ atmosphere, and the resulting suspension was degassed under vacuum and purged with H₂ several times. The reaction mixture was then stirred under H₂ (15 psi) at 25° C. for 16 hours. The mixture was filtered, and the filtrate was concentrated in vacuum. The resulting residue was purified by silica gel chromatography (5-10% CH₃OH in CH₂Cl₂) to afford tert-butyl 2-(4-piperidylmethoxy)acetate (1.8 g, 7.85 mmol, 92% yield) as a colorless oil. ¹H-NMR (400 MHz, DMSO-d₆) δ 3.93 (s, 2H), 3.48-3.34 (m, 3H), 3.26 (d, J=6.0 Hz, 2H), 2.94 (d, J=12.0 Hz, 2H), 1.60 (d, J=12.0 Hz, 3H), 1.45-1.40 (m, 9H), 1.18-0.95 (m, 2H).

### Step 3: Preparation of tert-butyl 2-((1-((1S,2S)-2-hydroxycyclopentyl)piperidin-4-yl)methoxy)acetate and tert-butyl 2-((1-((1R,2R)-2-hydroxycyclopentyl)piperidin-4-yl)methoxy)acetate

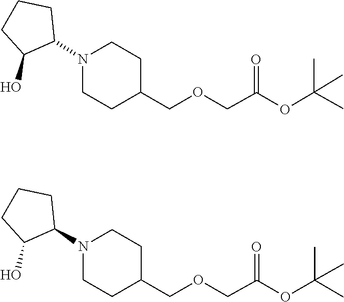 [SMILES: CC(C)(C)OC(=O)COCC1CCN([C@H]2CCC[C@@H]2O)CC1 ; CC(C)(C)OC(=O)COCC1CCN([C@@H]2CCC[C@H]2O)CC1]

To tert-butyl 2-(4-piperidylmethoxy)acetate (1.8 g, 7.85 mmol, 1 eq) in ethanol (20 mL) was added 6-oxabicyclo[3.1.0]hexane (1.3 g, 15.7 mmol, 2 eq), and the reaction mixture was stirred at 80° C. for 3 hours. The mixture was concentrated in vacuum, and the resulting residue was purified by silica gel chromatography (10% CH₃OH in CH₂Cl₂) to afford tert-butyl 2-[[1-(2-hydroxycyclopentyl)-4-piperidyl]methoxy]acetate (1.8 g, 5.74 mmol, 73% yield) as a yellow oil. ¹H-NMR (400 MHz, DMSO-d₆) δ 4.50 (s, 1H), 3.98-3.83 (m, 3H), 3.33-3.24 (m, 3H), 3.14-3.04 (m, 1H), 2.92-2.80 (m, 1H), 2.43-2.34 (m, 1H), 2.07-1.85 (m, 2H), 1.83-1.70 (m, 2H), 1.67-1.51 (m, 4H), 1.43 (s, 9H), 1.41-1.38 (m, 2H), 1.22-1.08 (m, 2H).

### Step 4: Preparation of tert-butyl 4-(7-bromo-2-(((1R,2R)-2-(4-((2-(tert-butoxy)-2-oxoethoxy)methyl)piperidin-1-yl)cyclopentyl)oxy)-6-chloro-8-fluoroquinazolin-4-yl)piperazine-1-carboxylate and tert-butyl 4-(7-bromo-2-(((1S,2S)-2-(4-((2-(tert-butoxy)-2-oxoethoxy)methyl)piperidin-1-yl)cyclopentyl)oxy)-6-chloro-8-fluoroquinazolin-4-yl)piperazine-1-carboxylate

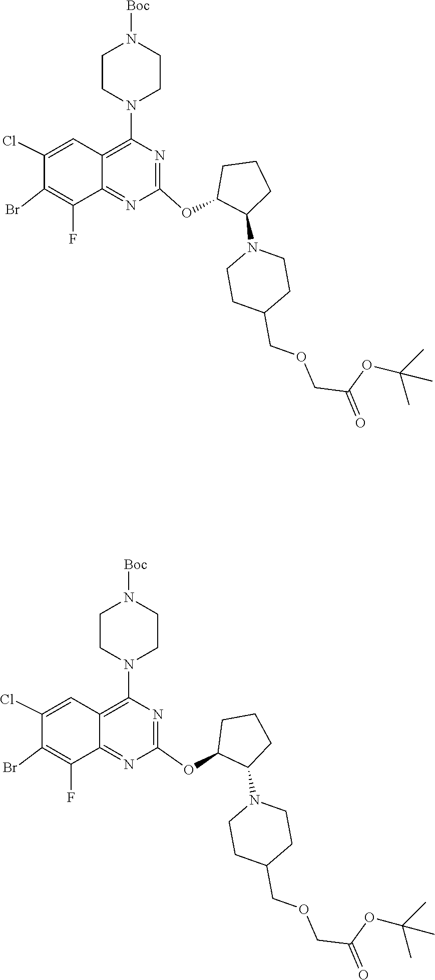 [SMILES: CC(C)(C)OC(=O)COCC1CCN([C@@H]2CCC[C@H]2Oc2nc(N3CCN(C(=O)OC(C)(C)C)CC3)c3cc(Cl)c(Br)c(F)c3n2)CC1 ; CC(C)(C)OC(=O)COCC1CCN([C@H]2CCC[C@@H]2Oc2nc(N3CCN(C(=O)OC(C)(C)C)CC3)c3cc(Cl)c(Br)c(F)c3n2)CC1]

To a mixture of tert-butyl4-(7-bromo-2,6-dichloro-8-fluoro-quinazolin-4-yl)piperazine-1-carboxylate (3.5 g, 7.18 mmol, 1.5 eq), tert-butyl 2-[[1-(2-hydroxycyclopentyl)-4-piperidyl]methoxy]acetate (1.5 g, 4.79 mmol, 1 eq), and 1,4-diazabicyclo[2.2.2]octane (54 mg, 0.48 mmol, 0.1 eq) in CH₃CN (10 mL) was added Cs₂CO₃ (3.1 g, 9.57 mmol, 2 eq), and the reaction mixture was stirred at 50° C. for 16 hours. The mixture was filtered, and the filtrate concentrated in vacuum. The resulting residue was purified by prep-TLC (50% EtOAc in petroleum ether) followed by prep-HPLC (75-100% CH₃CN in water (10 mM NH₄HCO₃)). Additional purification by SFC afforded tert-butyl 4-[7-bromo-2-[(1R,2R)-2-[4-[(2-tert-butoxy-2-oxo-ethoxy)methyl]-1-piperidyl]cyclopentoxy]-6-chloro-8-fluoro-quinazolin-4-yl]piperazine-1-carboxylate (400 mg, 0.53 mmol, 11% yield) as a yellow solid and tert-butyl 4-[7-bromo-2-[(1S,2S)-2-[4-[(2-tert-butoxy-2-oxo-ethoxy)methyl]-1-piperidyl]cyclopentoxy]-6-chloro-8-fluoro-quinazolin-4-yl]piperazine-1-carboxylate (400 mg, 0.53 mmol, 11% yield) as a yellow solid.

tert-butyl 4-[7-bromo-2-[(1R,2R)-2-[4-[(2-tert-butoxy-2-oxo-ethoxy)methyl]-1-piperidyl]cyclopentoxy]-6-chloro-8-fluoro-quinazolin-4-yl]piperazine-1-carboxylate: SFC RT=2.021 min

tert-butyl 4-[7-bromo-2-[(1S,2S)-2-[4-[(2-tert-butoxy-2-oxo-ethoxy)methyl]-1-piperidyl]cyclopentoxy]-6-chloro-8-fluoro-quinazolin-4-yl]piperazine-1-carboxylate: SFC RT=2.176 min

### Step 5: Preparation of tert-butyl 4-[2-[(1R,2R)-2-[4-[(2-tert-butoxy-2-oxo-ethoxy)methyl]-1-piperidyl]cyclopentoxy]-6-chloro-8-fluoro-7-(3-hydroxy-1-naphthyl)quinazolin-4-yl]piperazine-1-carboxylate

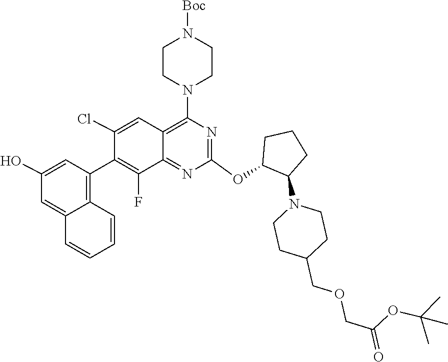 [SMILES: CC(C)(C)OC(=O)COCC1CCN([C@@H]2CCC[C@H]2Oc2nc(N3CCN(C(=O)OC(C)(C)C)CC3)c3cc(Cl)c(-c4cc(O)cc5ccccc45)c(F)c3n2)CC1]

To a mixture of tert-butyl4-[7-bromo-2-[(1R,2R)-2-[4-[(2-tert-butoxy-2-oxo-ethoxy)methyl]-1-piperidyl]cyclopentoxy]-6-chloro-8-fluoro-quinazolin-4-yl]piperazine-1-carboxylate (400 mg, 0.53 mmol, 1 eq), methanesulfonato (2-dicyclohexylphosphino-2,4,6-tri-i-propyl-1,1-biphenyl)(2-amino-1,1-biphenyl-2-yl)palladium(II) (22 mg, 0.03 mmol, 0.05 eq), and 4-(4,4,5,5-tetramethyl-1,3,2-dioxaborolan-2-yl)naphthalen-2-ol (128 mg, 0.48 mmol, 0.9 eq) in THF (10 mL) was added K₃PO₄ (1.5 N in water, 3 eq) under nitrogen, and the reaction mixture was stirred at 50° C. for 16 hours. Water (30 mL) was then added, and the resulting mixture was extracted with EtOAc (3×20 mL). The combined organic extracts were washed with brine (3×20 mL), dried over anhydrous Na₂SO₄, filtered, and concentrated. The resulting residue was purified by prep-TLC (10% CH₃OH in CH₂Cl₂) to afford tert-butyl 4-[2-[(1R,2R)-2-[4-[(2-tert-butoxy-2-oxo-ethoxy)methyl]-1-piperidyl]cyclopentoxy]-6-chloro-8-fluoro-7-(3-hydroxy-1-naphthyl)quinazolin-4-yl]piperazine-1-carboxylate (210 mg, 0.26 mmol, 48% yield) as a yellow solid. LC/MS (ESI) m/z: 820.4 [M+H]⁺.

### Step 6: Preparation of 2-[[1-[(1R,2R)-2-[4-(4-tert-butoxycarbonylpiperazin-1-yl)-6-chloro-8-fluoro-7-(3-hydroxy-1-naphthyl)quinazolin-2-yl]oxycyclopentyl]-4-piperidyl]methoxy]acetic Acid

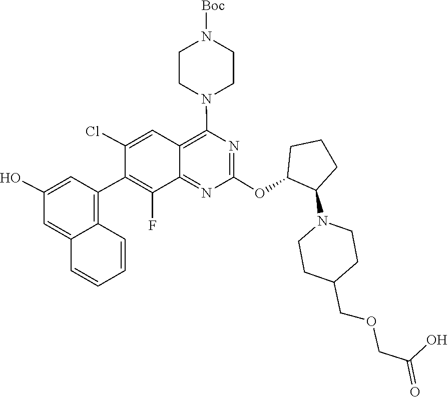 [SMILES: CC(C)(C)OC(=O)N1CCN(c2nc(O[C@@H]3CCC[C@H]3N3CCC(COCC(=O)O)CC3)nc3c(F)c(-c4cc(O)cc5ccccc45)c(Cl)cc23)CC1]

A mixture of tert-butyl 4-[2-[(1R,2R)-2-[4-[(2-tert-butoxy-2-oxo-ethoxy)methyl]-1-piperidyl]cyclopentoxy]-6-chloro-8-fluoro-7-(3-hydroxy-1-naphthyl)quinazolin-4-yl]piperazine-1-carboxylate (150 mg, 0.18 mmol, 1 eq) and LiOH (252 mg, 6 mmol, 33 eq) in CH₃OH (2 mL), H₂O (2 mL), and THF (2 mL), was stirred at 25° C. for 1 hour. The pH of the reaction was adjusted to 6 by addition of 1H HCl (10 mL), and the resulting mixture was extracted with EtOAc (3×30 mL). The combined organic extracts were washed with brine (3×30 mL), dried over anhydrous Na₂SO₄, filtered, and concentrated to afford 2-[[1-[(1R,2R)-2-[4-(4-tert-butoxycarbonylpiperazin-1-yl)-6-chloro-8-fluoro-7-(3-hydroxy-1-naphthyl)quinazolin-2-yl]oxycyclopentyl]-4-piperidyl]methoxy]acetic acid (130 mg, 0.17 mmol, 93% yield) as a yellow solid. LC/MS MS (ESI) m/z: 764.4 [M+H]⁺.

### Step 7: Preparation of tert-butyl 4-[6-chloro-8-fluoro-2-[(1R,2R)-2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]cyclopentoxy]-7-(3-hydroxy-1-naphthyl)quinazolin-4-yl]piperazine-1-carboxylate

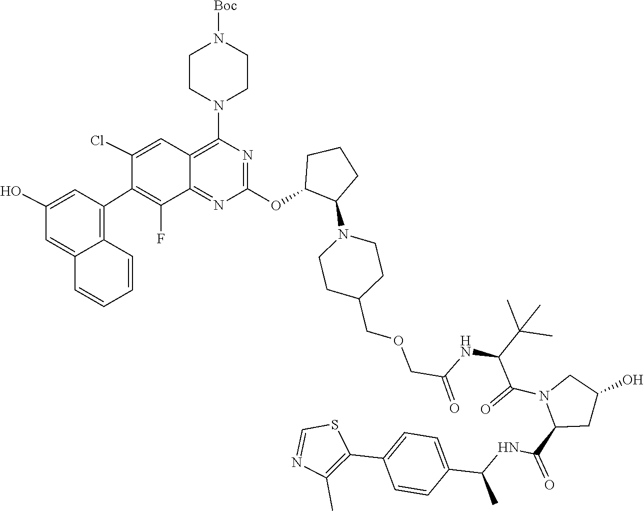 [SMILES: Cc1ncsc1-c1ccc([C@H](C)NC(=O)[C@@H]2C[C@@H](O)CN2C(=O)[C@@H](NC(=O)COCC2CCN([C@@H]3CCC[C@H]3Oc3nc(N4CCN(C(=O)OC(C)(C)C)CC4)c4cc(Cl)c(-c5cc(O)cc6ccccc56)c(F)c4n3)CC2)C(C)(C)C)cc1]

To a mixture of 2-[[1-[(1R,2R)-2-[4-(4-tert-butoxycarbonylpiperazin-1-yl)-6-chloro-8-fluoro-7-(3-hydroxy-1-naphthyl)quinazolin-2-yl]oxycyclopentyl]-4-piperidyl]methoxy]acetic acid (130 mg, 0.17 mmol, 1 eq), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (98 mg, 0.20 mmol, 1.2 eq, hydrochloride), 1-hydroxybenzotriazole (46 mg, 0.34 mmol, 2 eq), and N-(3-dimethylaminopropyl)-N-ethylcarbodiimide hydrochloride (65 mg, 0.34 mmol, 2 eq) in DMF (10 mL) was added diisopropylethylamine (66 mg, 0.51 mmol, 3 eq), and the reaction mixture was stirred at 25° C. for 16 hours. Water (20 mL) was then added, and the resulting mixture was extracted with EtOAc (3×30 mL). The combined organic extracts were washed with brine (3×30 mL), dried over anhydrous Na₂SO₄, filtered, and concentrated. The resulting residue was purified by prep-TLC (10% CH₃OH in CH₂Cl₂) to afford tert-butyl 4-[6-chloro-8-fluoro-2-[(1R,2R)-2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]cyclopentoxy]-7-(3-hydroxy-1-naphthyl)quinazolin-4-yl]piperazine-1-carboxylate (30 mg, 0.03 mmol, 15% yield) as a yellow solid. LC/MS (ESI) m/z: 1190.6 [M+H]⁺.

### Step 8: Preparation of (2S,4R)-1-[(2S)-2-[[2-[[1-[(1R,2R)-2-[6-chloro-8-fluoro-7-(3- hydroxy-1-naphthyl)-4-piperazin-1-yl-quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

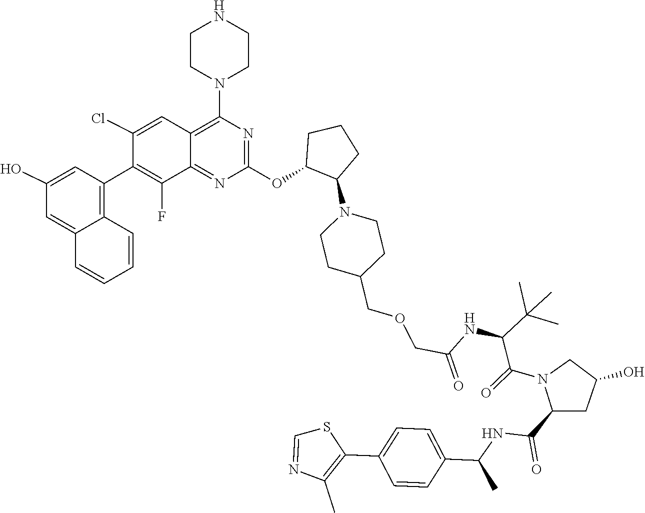 [SMILES: Cc1ncsc1-c1ccc([C@H](C)NC(=O)[C@@H]2C[C@@H](O)CN2C(=O)[C@@H](NC(=O)COCC2CCN([C@@H]3CCC[C@H]3Oc3nc(N4CCNCC4)c4cc(Cl)c(-c5cc(O)cc6ccccc56)c(F)c4n3)CC2)C(C)(C)C)cc1]

To tert-butyl 4-[6-chloro-8-fluoro-2-[(1R,2R)-2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]cyclopentoxy]-7-(3-hydroxy-1-naphthyl)quinazolin-4-yl]piperazine-1-carboxylate (30 mg, 0.03 mmol, 1 eq) in CH₂Cl₂ (5 mL) was added trifluoroacetic acid (1.5 g, 13.5 mmol, 536 eq), and the reaction mixture was stirred at 25° C. for 0.5 hour. The mixture was concentrated to afford (2S,4R)-1-[(2S)-2-[[2-[[1-[(1R,2R)-2-[6-chloro-8-fluoro-7-(3-hydroxy-1-naphthyl)-4-piperazin-1-yl-quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (30 mg, 0.02 mmol, 99% yield, trifluoroacetic acid salt) as a yellow solid. LC/MS (ESI) m/z: 1090.5 [M+H]⁺.

### Step 9: Preparation of (2S,4R)-1-[(2S)-2-[[2-[[1-[(1R,2R)-2-[6-chloro-8-fluoro-7-(3- hydroxy-1-naphthyl)-4-(4-prop-2-enoylpiperazin-1-yl)quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

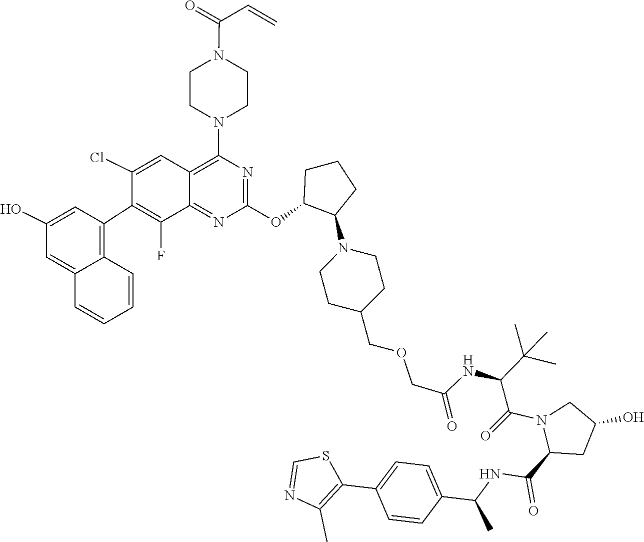 [SMILES: C=CC(=O)N1CCN(c2nc(O[C@@H]3CCC[C@H]3N3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To a mixture of (2S,4R)-1-[(2S)-2-[[2-[[1-[(1R,2R)-2-[6-chloro-8-fluoro-7-(3-hydroxy-1-naphthyl)-4-piperazin-1-yl-quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (30 mg, 0.02 mmol, 1 eq, trifluoroacetic acid salt) and 2,6-lutidine (27 mg, 0.25 mmol, 10 eq) in DMF (5 mL) at −78° C. was added prop-2-enoyl chloride (2 mg, 0.02 mmol, 1 eq), and the reaction mixture was stirred at −78° C. for 0.5 hour. Water (20 mL) was then added, and the resulting mixture was extracted with EtOAc (3×20 mL). The combined organic extracts were washed with brine (3×30 mL), dried over anhydrous Na₂SO₄, filtered, and concentrated. The resulting residue was purified by prep-HPLC (28-58% CH₃CN in water (0.225% formic acid)) to afford (2S,4R)-1-[(2S)-2-[[2-[[1-[(1R,2R)-2-[6-chloro-8-fluoro-7-(3-hydroxy-1-naphthyl)-4-(4-prop-2-enoylpiperazin-1-yl)quinazolin-2-yl]oxycyclopentyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (12.7 mg, 0.01 mmol, 43% yield, 97% purity) as a white solid. LC/MS (ESI) m/z: 572.8 [1/2M+H]⁺. ¹H-NMR (400 MHz, DMSO-d₆) δ 8.99 (s, 1H), 8.45 (d, J=7.6 Hz, 1H), 8.30 (s, 1H), 8.01 (s, 1H), 7.81 (d, J=8.4 Hz, 1H), 7.50-7.13 (m, 9H), 7.08 (d, J=2.0 Hz, 1H), 6.85-6.81 (m, 1H), 6.20-6.16 (m, 1H), 5.80-5.71 (m, 1H), 5.36 (d, J=3.6 Hz, 1H), 4.91-4.84 (m, 1H), 4.56-4.41 (m, 2H), 4.32-4.25 (m, 1H), 4.01-3.76 (m, 12H), 3.01-2.88 (m, 3H), 2.45 (s, 3H), 2.14-1.84 (m, 6H), 1.80-1.07 (m, 15H), 0.90 (s, 9H).

The following compounds can be prepared in an analogous manner to (2S,4R)-1-[(2S)-2-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4R)-1-[(2S)-2-[[2-[2-[[1-[(2S)-2-[6-chloro-8-fluoro-7-(3-hydroxy-1- naphthyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

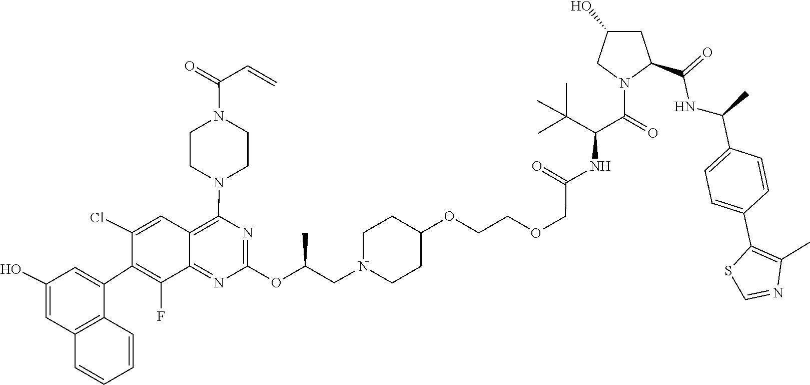 [SMILES: C=CC(=O)N1CCN(c2nc(O[C@@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

LC/MS (ESI) m/z: 1148.4 [M]⁺. ¹H-NMR (400 MHz, DMSO-d₆) δ 10.01 (s, 1H), 8.99 (s, 1H), 8.42-8.40 (m, 1H), 8.01 (s, 1H), 7.82-7.75 (m, 1H), 7.48-7.41 (m, 3H), 7.38-7.29 (m, 5H), 7.25-7.17 (m, 2H), 7.08-7.05 (m, 1H), 6.84-6.8 (m, 1H), 6.19-6.10 (m, 1H), 5.79-5.72 (m, 1H), 5.44-5.36 (m, 1H), 5.12-5.09 (m, 1H), 4.90-4.80 (m, 1H), 4.53-4.50 (m, 1H), 4.44-4.31 (m, 1H), 4.29 (s, 1H), 3.95-3.91 (m, 4H), 3.86 (s, 2H), 3.78 (s, 2H), 3.63-3.50 (m, 6H), 3.29-3.15 (m, 1H), 2.88-2.72 (m, 2H), 2.66-2.59 (m, 2H), 2.47-2.44 (m, 1H), 2.46 (s, 3H), 1.77-1.63 (m, 3H), 1.39-1.15 (m, 10H), 0.91 (s, 10H).

### 2. (2S,4R)-1-[(2S)-2-[[2-[2-[2-[2-[2-[2-[2-[[1-[(2R)-2-[6- chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

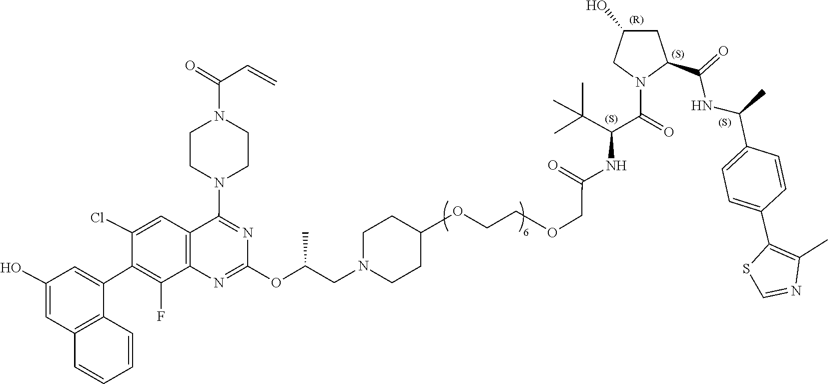

LC/MS (ESI) m/z: 1368.7 [M+H]⁺. ¹H-NMR (400 MHz, DMSO-d₆) δ 10.04 (br s, 1H), 9.30 (br s, 1H), 8.98 (s, 1H), 8.79-8.36 (m, 1H), 8.07 (s, 1H), 7.81 (d, J=8.3 Hz, 1H), 7.47-7.41 (m, 3H), 7.40-7.33 (m, 3H), 7.30 (d, J=2.1 Hz, 1H), 7.25-7.16 (m, 2H), 7.07 (dd, J=2.3, 9.2 Hz, 1H), 6.83 (dd, J=10.4, 16.7 Hz, 1H), 6.19 (dd, J=2.2, 16.7 Hz, 1H), 5.82-5.69 (m, 1H), 5.63 (br s, 1H), 4.90 (br t, J=7.1 Hz, 1H), 4.54 (br d, J=9.5 Hz, 1H), 4.43 (br t, J=8.1 Hz, 1H), 4.31-4.22 (m, 1H), 4.00-3.95 (m, 6H), 3.91 (br s, 2H), 3.63-3.55 (m, 10H), 3.52 (br d, J=3.1 Hz, 5H), 3.49 (br d, J=2.4 Hz, 16H), 3.18-2.97 (m, 3H), 2.45 (s, 3H), 2.12-2.02 (m, 3H), 1.91 (br s, 2H), 1.77 (m, J=4.6, 8.6, 12.9 Hz, 2H), 1.46-1.37 (m, 6H), 0.93 (s, 9H).

### 3. (2S,4R)-1-[(2S)-2-[[2-[2-[2-[2-[2-[2-[2-[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]ethoxy]ethoxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

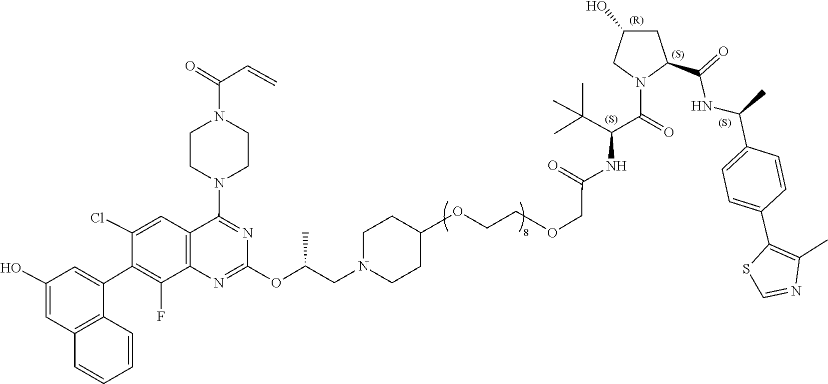

LC/MS (ESI) m/z: 1457.8 [M+H]⁺. ¹H-NMR (400 MHz, DMSO-d₆) δ 10.07 (s, 1H), 8.98 (s, 1H), 8.43 (d, J=7.6 Hz, 1H), 8.20 (s, 1H), 8.01 (s, 1H), 7.81 (d, J=8.4 Hz, 1H), 7.50-7.41 (m, 1H), 7.50-7.41 (m, 1H), 7.50-7.41 (m, 1H), 7.50-7.33 (m, 1H), 7.29 (d, J=2.4 Hz, 1H), 7.25-7.14 (m, 2H), 7.07 (dd, J=2.4, 5.2 Hz, 1H), 6.84 (dd, J=10.4, 16.8 Hz, 1H), 6.24-6.12 (m, 1H), 5.80-5.70 (m, 1H), 5.39 (qd, J=6.0, 11.6 Hz, 1H), 5.13 (br s, 1H), 4.95-4.86 (m, 1H), 4.55 (d, J=9.6 Hz, 1H), 4.45 (t, J=8.0 Hz, 1H), 4.29 (br s, 1H), 4.01-3.74 (m, 10H), 3.65-3.44 (m, 30H), 3.22 (br dd, J=4.4, 9.2 Hz, 5H), 2.94-2.70 (m, 3H), 2.65-2.56 (m, 2H), 2.46 (s, 3H), 2.42-2.35 (m, 1H), 2.16-1.99 (m, 3H), 1.83-1.67 (m, 3H), 1.48-1.36 (m, 3H), 1.31 (dd, J=1.6, 6.4 Hz, 3H), 1.27-1.21 (m, 1H), 0.94 (s, 9H).

### 4. (2S,4R)-1-[(2S)-2-[[2-[2-[2-[2-[2-[2-[2-[2-[2-[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]ethoxy]ethoxy]ethoxy]ethoxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

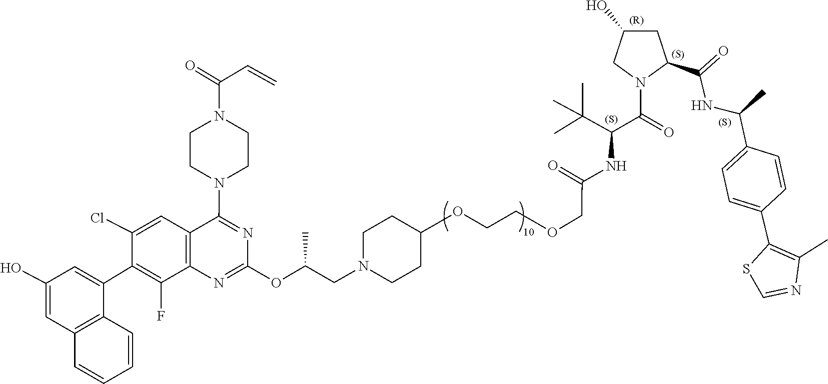

LC/MS (ESI) m/z: 773.0 [M/2+1]⁺. ¹H-NMR (400 MHz, DMSO-d₆) δ 10.07 (s, 1H), 9.24 (s, 1H), 8.99 (s, 1H), 8.43-8.2 (m, 1H), 8.08 (s, 1H), 7.71-7.6 (m, 1H), 7.48-7.42 (m, 3H), 7.37-7.2 (m, 3H), 7.30-7.28 (m, 1H), 7.25-7.17 (m, 2H), 7.07-7.0 (m, 1H), 6.85-6.8 (m, 1H), 6.24-6.16 (m, 1H), 5.77-5.68 (m, 1H), 5.65 (s, 1H), 4.94-4.89 (m, 1H), 4.55-4.5 (m, 1H), 4.45-4.32 (m, 1H), 4.29 (s, 1H), 4.06-3.95 (m, 4H), 3.87 (s, 3H), 3.80 (s, 3H), 3.50-3.2 (m, 34H), 3.15-2.9 (m, 2H), 2.71-2.66 (m, 3H), 2.57-2.5 (s, 1H), 2.46 (s, 8H), 2.36-2.31 (m, 2H), 2.13-2.01 (m, 2H), 1.91-1.85 (s, 2H), 1.81-1.60 (m, 2H), 1.38-1.2 (m, 9H), 0.94 (s, 9H).

### 5. (2S,4R)-1-[(2S)-2-[[2-[2-[[1-[(2R)-2-[6-chloro-8-fluoro-7-(3-hydroxy-1- naphthyl)-4-[(2S)-2-methyl-4-prop-2-enoyl-piperazin-1-yl]quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

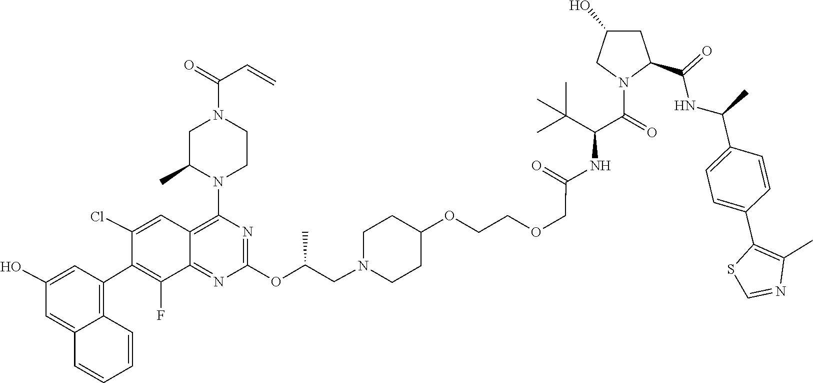 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)[C@@H](C)C1]

(formic acid salt, white solid). LC/MS (ESI) m/z: 1162.6 [M+H]⁺. ¹H-NMR (400 MHz, CD₃OD) δ 8.91-8.81 (s, 1H), 8.56-8.48 (s, 1H), 7.95 (s, 1H), 7.79-7.67 (m, 1H), 7.58-7.34 (m, 5H), 7.28-7.14 (m, 3H), 7.06-7.00 (m, 1H), 6.91-6.74 (m, 1H), 6.37-6.23 (d, J=13.2 Hz, 1H), 5.86-5.79 (d, J=12.4 Hz, 1H), 5.68-5.58 (m, 1H), 4.98-4.96 (m, 1H), 4.70-4.66 (m, 1H), 4.62-4.50 (m, 2H), 4.46-4.14 (m, 4H), 4.07-3.98 (m, 2H), 3.86-3.71 (m, 3H), 3.70-3.48 (m, 7H), 3.26-3.22 (m, 1H), 3.13 (s, 2H), 3.00-2.65 (m, 3H), 2.46 (d, J=3.2 Hz, 3H), 2.20 (dd, J=7.6, 13.2 Hz, 1H), 2.04-1.86 (m, 3H), 1.82-1.64 (m, 2H), 1.55 (d, J=6.8 Hz, 1H), 1.49-1.34 (m, 8H), 1.06-0.88 (s, 9H).

### 6. (2S,4R)-1-[(2S)-2-[[2-[2-[[1-[(2R)-2-[6-chloro-8-fluoro-7-(3-hydroxy-1- naphthyl)-4-[(3R)-3-methyl-4-prop-2-enoyl-piperazin-1-yl]quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

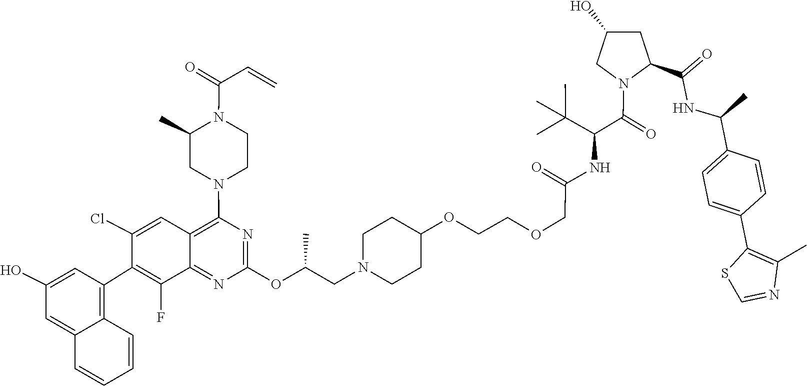 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@H]1C]

(formic acid salt, white solid). LC/MS (ESI) m/z: 1162.6 [M+H]⁺. ¹H-NMR (400 MHz, CD₃OD) δ 8.90-8.82 (s, 1H), 8.66-8.44 (m, 1H), 8.02 (s, 1H), 7.74 (d, J=8.0 Hz, 1H), 7.45-7.34 (m, 5H), 7.30-7.14 (m, 3H), 7.07-6.99 (m, 1H), 6.79 (dd, J=10.8, 16.0 Hz, 1H), 6.27 (d, J=16.4 Hz, 1H), 5.80 (d, J=10.8 Hz, 1H), 5.64 (s, 1H), 4.97 (dd, J=3.2, 6.8 Hz, 1H), 4.65-4.51 (m, 4H), 4.43 (s, 2H), 4.25 (d, J=13.2 Hz, 1H), 4.08-3.96 (m, 2H), 3.83 (d, J=11.2 Hz, 2H), 3.74 (dd, J=3.6, 11.2 Hz, 1H), 3.70-3.40 (m, 7H), 3.24-2.51 (m, 6H), 2.46 (d, J=2.8 Hz, 3H), 2.20 (m, 1H), 2.06-1.85 (m, 3H), 1.82-1.27 (m, 11H), 1.06-0.91 (s, 9H).

### 7. (2S,4R)-1-[(2S)-2-[[2-[[1-[2-[6-chloro-4-[(2S,5R)-2,5-dimethyl-4-prop-2- enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

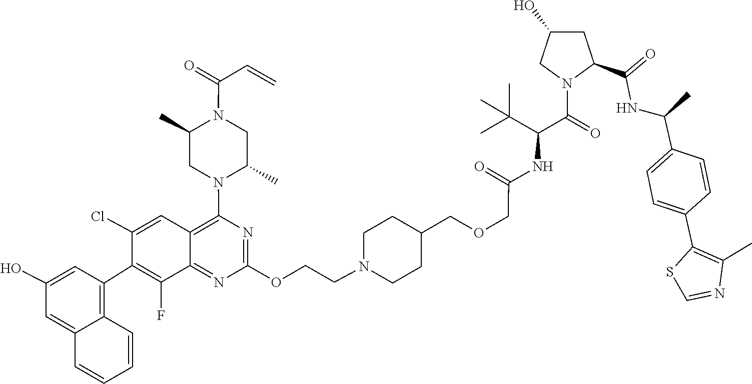 [SMILES: C=CC(=O)N1C[C@H](C)N(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@H]1C]

(formic acid salt, white solid). LCMS (ESI) m/z: 1132.4 [M+H]⁺. ¹H-NMR (400 MHz, CD₃OD) δ 8.88-8.84 (m, 1H), 8.52 (s, 1H), 8.01-7.95 (m, 1H), 7.78-7.72 (m, 1H), 7.45-7.35 (m, 5H), 7.29-7.15 (m, 3H), 7.11-7.01 (m, 1H), 6.91-6.73 (m, 1H), 6.35-6.24 (m, 1H), 5.86-5.78 (m, 1H), 4.98 (d, J=2.0 Hz, 1H), 4.70 (d, J=13.2 Hz, 4H), 4.43 (m, 4H), 4.07-3.40 (m, 9H), 3.35 (s, 1H), 3.24-3.11 (m, 3H), 2.62-2.51 (m, 1H), 2.47 (s, 3H), 2.40-2.12 (m, 1H), 2.05-1.73 (m, 4H), 1.61-1.10 (m, 12H), 1.06-0.97 (m, 9H).

### 8. (2S,4R)-1-[(2S)-2-[[2-[2-[[1-[(2R)-2-[6-chloro-8-fluoro-7-(2-fluoro-6- hydroxy-phenyl)-4-(4-prop-2-enoylpiperazin-1-yl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

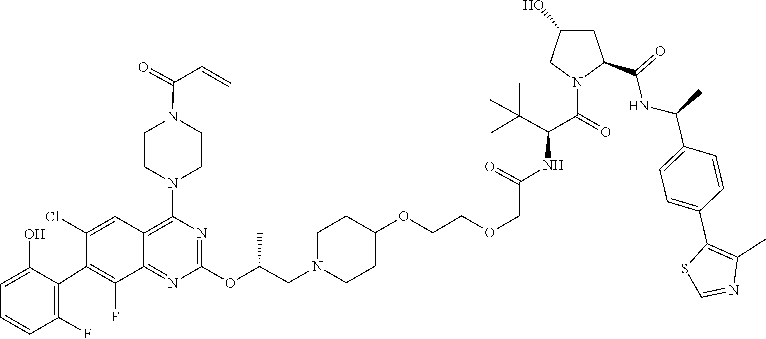 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(O)cccc4F)c(Cl)cc23)CC1]

(formic acid salt, white solid). LC/MS (ESI) m/z: 1116.5 [M+H]⁺. ¹H-NMR (400 MHz, CD₃OD) δ 8.93-8.77 (m, 1H), 8.53 (s, 1H), 7.94 (s, 1H), 7.52-7.18 (m, 5H), 6.88-6.63 (m, 3H), 6.28 (dd, J=1.6, 16.8 Hz, 1H), 5.81 (d, J=12.0 Hz, 1H), 5.68-5.55 (m, 1H), 4.99 (dd, J=3.2, 7.2 Hz, 1H), 4.84-4.76 (m, 1H), 4.69 (s, 1H), 4.58-4.56 (m, 1H), 4.55 (d, J=3.2 Hz, 1H), 4.44 (s, 1H), 4.09-4.02 (m, 2H), 3.99 (d, J=3.6 Hz, 4H), 3.90 (s, 4H), 3.86-3.80 (m, 1H), 3.78-3.43 (m, 7H), 3.10 (s, 3H), 3.00-2.63 (m, 2H), 2.50-2.44 (m, 3H), 2.21 (dd, J=8.0, 12.7 Hz, 1H), 2.07-1.63 (m, 5H), 1.60-1.37 (m, 6H), 1.06-0.97 (m, 9H).

### 9. (2S,4R)-1-[(2S)-2-[[2-[2-[[1-[(2R)-2-[6-chloro-8-fluoro-7-(2-fluoro-6- hydroxy-phenyl)-4-[(3R)-3-methyl-4-prop-2-enoyl-piperazin-1-yl]quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

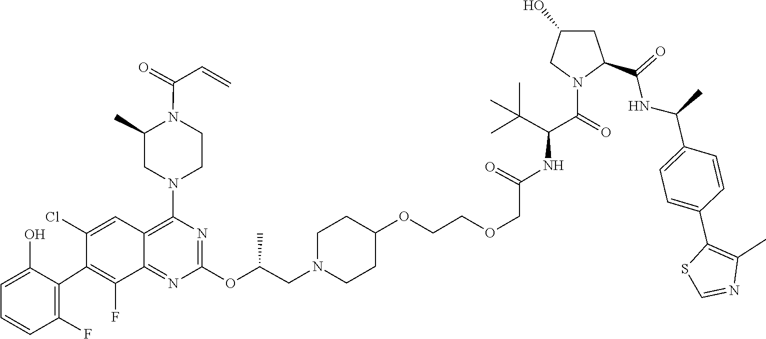 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(O)cccc4F)c(Cl)cc23)C[C@H]1C]

(formic acid salt, white solid). LC/MS (ESI) m/z: 1130.5 [M+H]⁺. ¹H-NMR (400 MHz, CD₃OD) δ 8.91-8.82 (s, 1H), 8.53 (s, 1H), 8.00-7.89 (m, 1H), 7.47-7.24 (m, 5H), 6.87-6.65 (m, 3H), 6.31-6.23 (d, J=16.4 Hz, 1H), 5.79 (d, J=4.8 Hz, 1H), 5.68-5.56 (m, 1H), 4.98 (d, J=7.2 Hz, 1H), 4.59-4.52 (m, 4H), 4.47-4.33 (m, 2H), 4.28-4.18 (m, 1H), 4.09-3.98 (m, 2H), 3.87-3.71 (m, 3H), 3.71-3.42 (m, 7H), 3.15-2.86 (m, 3H), 2.81-2.52 (m, 2H), 2.50-2.45 (s, 3H), 2.20 (m, 1H), 2.02-1.85 (m, 3H), 1.76-1.27 (m, 12H), 1.08-0.96 (s, 9H).

### Exemplary Synthesis of (2S,4R)-1-[(2S)-2-[[2-[[1-[(2R)-2-[6-chloro-8-fluoro-7-(3-hydroxy-1-naphthyl)-4-(4-prop-2-enoylpiperazin-1-yl) quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide

### Step 1: Preparation of tert-butyl 4-[6-chloro-8-fluoro-2-[(1R)-2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]-1-methyl-ethoxy]-7-(3-hydroxy-1-naphthyl)quinazolin-4-yl]piperazine-1-carboxylate

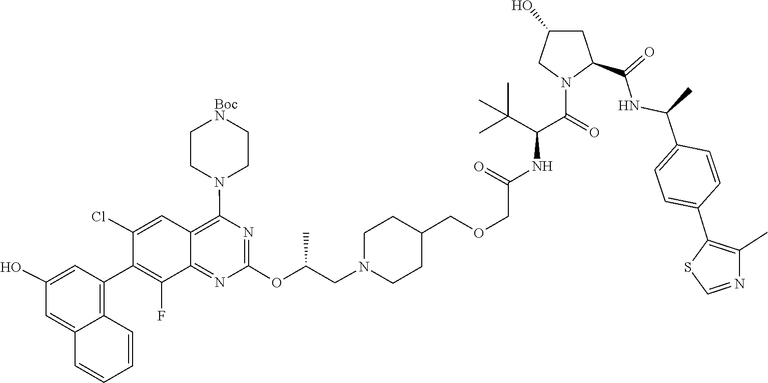 [SMILES: Cc1ncsc1-c1ccc([C@H](C)NC(=O)[C@@H]2C[C@@H](O)CN2C(=O)[C@@H](NC(=O)COCC2CCN(C[C@@H](C)Oc3nc(N4CCN(C(=O)OC(C)(C)C)CC4)c4cc(Cl)c(-c5cc(O)cc6ccccc56)c(F)c4n3)CC2)C(C)(C)C)cc1]

To (2S,4R)-1-[(2S)-3,3-dimethyl-2-[[2-(4-piperidylmethoxy) acetyl]amino]butanoyl]-4-hydroxy-N-[(1S)-1-[4-(4-methylthiazol-5-yl) phenyl]ethyl]pyrrolidine-2-carboxamide (90 mg, 0.14 mmol, 1 eq, HCl salt) in CH₃OH (2 mL) was added NaOAc (23 mg, 0.28 mmol, 2 eq), and the resulting mixture was stirred at 25° C. for 20 minutes. A solution of tert-butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (90 mg, 0.15 mmol, 1.10 eq) in CH₂Cl₂ (3 mL) was then added, and the resulting mixture was cooled to 0° C. NaBH₃CN (18 mg, 0.28 mmol, 2 eq) was added, and the reaction mixture was stirred at 0-25° C. for 14 hours. The reaction mixture was quenched by addition of water (1 mL) at 25° C. The resulting mixture was diluted with CH₂Cl₂ (5 mL) and extracted with water (3×5 mL). The organic extract was washed with brine (15 mL), dried over anhydrous Na₂SO₄, filtered, and concentrated. The resulting residue was purified by prep-TLC (10% CH₃OH in CH₂Cl₂) to afford 1 tert-butyl 4-[6-chloro-8-fluoro-2-[(1R)-2-[4-[[2-[[(1S)-1-[(2S,4R)-4-hydroxy-2-[[(1S)-1-[4-(4-methylthiazol-5-yl) phenyl]ethyl]carbamoyl]pyrrolidine-1-carbonyl]-2,2-dimethyl-propyl]amino]-2-oxo-ethoxy]methyl]-1-piperidyl]-1-methyl-ethoxy]-7-(3-hydroxy-1-naphthyl) quinazolin-4-yl]piperazine-1-carboxylate (53 mg, 0.045 mmol, 32% yield) as a yellow solid. LC/MS (ESI) m/z: 1164.5 [M+H]⁺.

### Step 2: Preparation of (2S,4R)-1-[(2S)-2-[[2-[[1-[(2R)-2-[6-chloro-8-fluoro-7-(3-hydroxy-1-naphthyl)-4-piperazin-1-yl-quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide

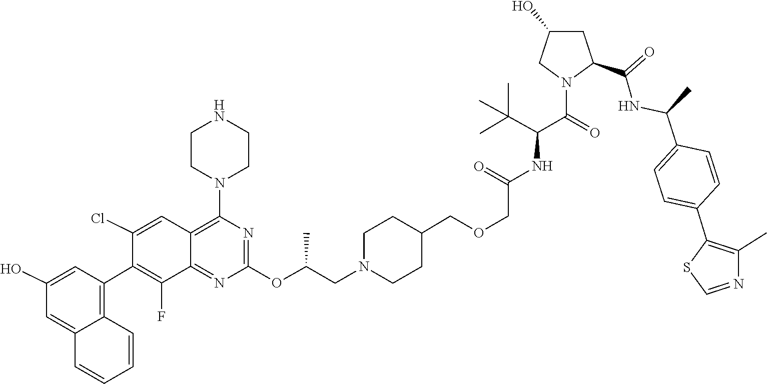 [SMILES: Cc1ncsc1-c1ccc([C@H](C)NC(=O)[C@@H]2C[C@@H](O)CN2C(=O)[C@@H](NC(=O)COCC2CCN(C[C@@H](C)Oc3nc(N4CCNCC4)c4cc(Cl)c(-c5cc(O)cc6ccccc56)c(F)c4n3)CC2)C(C)(C)C)cc1]

To a solution of tert-butyl 4-[6-chloro-8-fluoro-2-[(1R)-2-[4-[[2-[[(1S)-1-[(2S,4R)-4-hydroxy-2-[[(1S)-1-[4-(4-methylthiazol-5-yl) phenyl]ethyl]carbamoyl]pyrrolidine-1-carbonyl]-2,2-dimethyl-propyl]amino]-2-oxo-ethoxy]methyl]-1-piperidyl]-1-methyl-ethoxy]-7-(3-hydroxy-1-naphthyl) quinazolin-4-yl]piperazine-1-carboxylate (53 mg, 0.045 mmol, 1 eq) in CH₂Cl₂ (5 mL) was added TFA (770 mg, 6.75 mmol, 0.5 mL, 148.42 eq), and the reaction mixture was stirred at 25° C. for 2 hours. The reaction mixture was concentrated under reduced pressure to get (2S,4R)-1-[(2S)-2-[[2-[[1-[(2R)-2-[6-chloro-8-fluoro-7-(3-hydroxy-1-naphthyl)-4- piperazin-1-yl-quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide (53 mg, 0.045 mmol, 99% yield, TFA salt) as a yellow solid.

### Step 3: Preparation of (2S,4R)-1-[(2S)-2-[[2-[[1-[(2R)-2-[6-chloro-8-fluoro-7-(3-hydroxy-1-naphthyl)-4-(4-prop-2-enoylpiperazin-1-yl) quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide

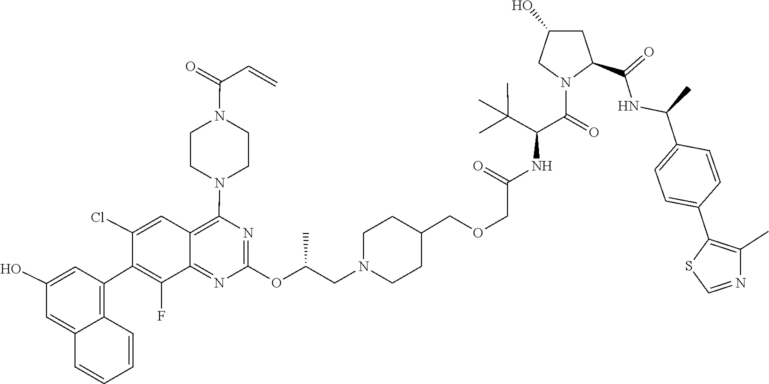 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To a mixture of (2S,4R)-1-[(2S)-2-[[2-[[1-[(2R)-2-[6-chloro-8-fluoro-7-(3-hydroxy-1-naphthyl)-4-piperazin-1-yl-quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide (53 mg, 0.045 mmol, 1 eq, trifluoroacetate) and 2,6-lutidine (96 mg, 0.89 mmol, 104.74 uL, 20 eq) in CH₂Cl₂ (5 mL) at −78° C. was added a solution of prop-2-enoyl chloride (4 mg, 0.040 mmol, 3.30 uL, 0.9 eq) in CH₂Cl₂ (0.5 mL), and the reaction mixture was stirred at −65° C. for 10 minutes. The reaction mixture was diluted with water (5 mL) and extracted with CH₂Cl₂ (4×5 mL). The combined organic extracts were dried over anhydrous Na₂SO₄, filtered, and concentrated. The resulting residue was purified by semi-preparative reverse phase HPLC (21-51% CH₃CN in water (0.1% trifluoroacetic acid)). The fractions containing the desired product were partially concentrated, and the resulting mixture was neutralized by addition of NaHCO₃. The aqueous mixture was then extracted with CH₂Cl₂ (3×5 mL), and the combined organic extracts were dried over anhydrous Na₂SO₄, filtered, and concentrated to afford (2S,4R)-1-[(2S)-2-[[2-[[1-[(2R)-2-[6-chloro-8-fluoro-7-(3-hydroxy-1-naphthyl)-4-(4-prop-2-enoylpiperazin-1-yl) quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide (5.8 mg, 0.0049 mmol, 11% yield, 96% purity) as a white solid after freeze-drying.

LC/MS (ESI) m/z: 1118.6 [M+H]⁺. ¹H-NMR (400 MHz, DMSO-d₆) δ 10.00 (s, 1H), 8.98 (s, 1H), 8.43 (d, J=7.6 Hz, 1H), 8.00 (s, 1H), 7.80 (d, J=8.3 Hz, 1H), 7.48-7.38 (m, 3H), 7.37-7.33 (m, 2H), 7.30-7.18 (m, 4H), 7.06 (dd, J=2.4, 4.2 Hz, 1H), 6.83 (dd, J=10.5, 16.6 Hz, 1H), 6.18 (dd, J=2.3, 16.7 Hz, 1H), 5.77-5.72 (m, 1H), 5.44-5.34 (m, 1H), 5.12 (d, J=2.9 Hz, 1H), 4.88 (quin, J=6.9 Hz, 1H), 4.52 (d, J=9.6 Hz, 1H), 4.46-4.38 (m, 1H), 4.30-4.23 (m, 1H), 3.92 (br s, 4H), 3.88 (s, 3H), 3.78 (br s, 2H), 3.62-3.52 (m, 2H), 3.48-3.38 (m, 1H), 3.30 (br s, 2H), 3.26 (br s, 1H), 3.00-2.88 (m, 2H), 2.64-2.59 (m, 1H), 2.45 (s, 3H), 2.41-2.37 (m, 1H), 2.07-1.96 (m, 3H), 1.75 (ddd, J=4.4, 8.7, 12.9 Hz, 1H), 1.56 (br s, 2H), 1.53-1.42 (m, 2H), 1.35 (dd, J=2.4, 6.9 Hz, 2H), 1.31 (br d, J=4.9 Hz, 2H), 1.23 (s, 1H), 1.16-1.07 (m, 2H), 0.90 (br d, J=1.6 Hz, 9H).

The following compound can be prepared in an analogous manner to (2S,4R)-1-[(2S)-2-[[2-[[1-[(2R)-2-[6-chloro-8-fluoro-7-(3-hydroxy-1-naphthyl)-4-(4-prop-2-enoylpiperazin-1-yl) quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide

### 1. (2S,4R)-1-[(2S)-2-[[2-[[1-[(2R)-2-[6-chloro-8-fluoro-7-(3-hydroxy-8-methyl-1- naphthyl)-4-(4-prop-2-enoylpiperazin-1-yl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

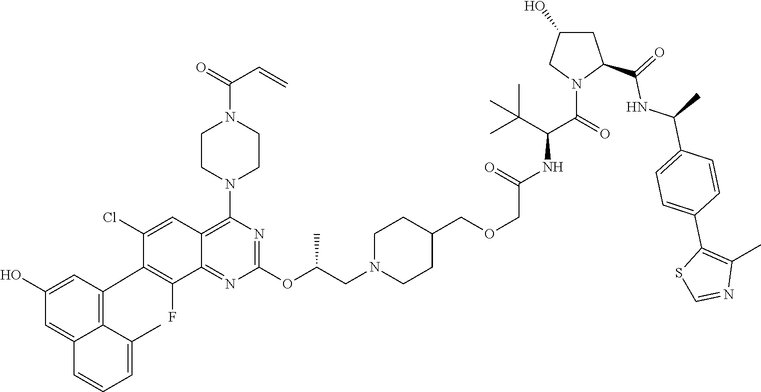 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5cccc(C)c45)c(Cl)cc23)CC1]

(formic acid salt, white solid) was obtained as a white solid. LC/MS (ESI) m/z: [M+H]⁺. ¹H-NMR (400 MHz, CD₃OD) δ 8.96-8.80 (m, 1H), 8.51 (s, 1H), 8.01 (br s, 1H), 7.63 (br d, J=7.9 Hz, 1H), 7.44-7.37 (m, 4H), 7.33-7.23 (m, 2H), 7.05 (br d, J=7.2 Hz, 1H), 6.89-6.78 (m, 2H), 6.29 (dd, J=1.7, 16.8 Hz, 1H), 5.85-5.79 (m, 1H), 5.68 (s, 1H), 5.07-4.97 (m, 1H), 4.74-4.25 (m, 2H), 4.08-3.88 (m, 10H), 3.86-3.71 (m, 2H), 3.58-3.47 (m, 1H), 3.45-3.34 (m, 4H), 3.21-3.05 (m, 1H), 2.94-2.57 (m, 2H), 2.50-2.44 (m, 3H), 2.42-2.13 (m, 1H), 2.03 (s, 3H), 1.96-1.78 (m, 3H), 1.59-1.26 (m, 10H), 1.03-0.99 (m, 9H).

### Exemplary Synthesis of (2S,4R)-1-[(2S)-2-[[2-[1-[(2R)-2-[6-chloro-8-fluoro-7-(3-hydroxy-1-naphthyl)-4-(4-prop-2-enoylpiperazin-1-yl)quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 3-(2-ethoxy-2-oxo-ethoxy)azetidine-1-carboxylate

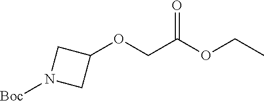 [SMILES: CCOC(=O)COC1CN(C(=O)OC(C)(C)C)C1]

To a mixture of tert-butyl 3-hydroxyazetidine-1-carboxylate (2.00 g, 11.55 mmol, 1.00 eq) and rhodium acetate (255 mg, 1.15 mmol, 0.10 eq) in CH₂Cl₂ (20 mL) at 0° C. was added ethyl 2-diazoacetate (13.17 g, 115.47 mmol, 12 mL, 10.00 eq) in CH₂Cl₂ (10 mL) dropwise, and the reaction mixture was stirred at 25° C. for 2 hours. The mixture was filtered, and the filtrate was concentrated. The resulting residue was purified by silica gel chromatography (0-50% EtOAc in petroleum ether) to afford tert-butyl 3-(2-ethoxy-2-oxo-ethoxy)azetidine-1-carboxylate (800 mg, 3.09 mmol, 26% yield) as a colorless oil. ¹H-NMR (400 MHz, CDCl₃) δ 4.34 (m, 1H), 4.25-4.22 (m, 2H), 4.13-4.08 (m, 2H), 4.05 (s, 2H), 3.96-3.90 (m, 2H), 1.46-1.43 (s, 9H), 1.30 (t, J=7.2 Hz, 3H).

### Step 2: Preparation of 2-(1-tert-butoxycarbonylazetidin-3-yl)oxyacetic Acid

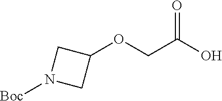 [SMILES: CC(C)(C)OC(=O)N1CC(OCC(=O)O)C1]

To a solution of tert-butyl 3-(2-ethoxy-2-oxo-ethoxy)azetidine-1-carboxylate (800 mg, 3.09 mmol, 1.00 eq) in CH₃OH (5 mL) and water (3 mL) was added LiOH (194 mg, 4.63 mmol, 1.50 eq), and the reaction mixture was stirred at 25° C. for 10 minutes. The mixture was neutralized by addition of 1N aqueous HCl at 25° C. The resulting mixture was concentrated, diluted with water (10 mL), and then extracted with EtOAc (3×5 mL). The combined organic extracts were concentrated under reduced pressure to afford 2-(1-tert-butoxycarbonylazetidin-3-yl)oxyacetic acid (260 mg, 1.12 mmol, 36% yield) as a colorless oil. ¹H-NMR (400 MHz, CDCl₃) δ 4.33-4.25 (m, 1H), 4.08-4.00 (m, 4H), 3.91-3.79 (m, 2H), 1.39-1.35 (s, 9H).

### Step 3: Preparation of tert-butyl 3-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]azetidine-1-carboxylate

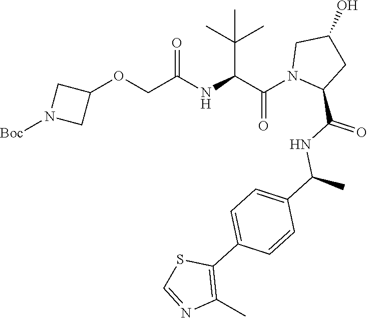 [SMILES: Cc1ncsc1-c1ccc([C@H](C)NC(=O)[C@@H]2C[C@@H](O)CN2C(=O)[C@@H](NC(=O)COC2CN(C(=O)OC(C)(C)C)C2)C(C)(C)C)cc1]

To a solution of 2-(1-tert-butoxycarbonylazetidin-3-yl)oxyacetic acid (120 mg, 0.52 mmol, 1.15 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (200 mg, 0.45 mmol, 1.00 eq) in DMF (3 mL) were added 1-hydroxybenzotriazole (91 mg, 0.67 mmol, 1.50 eq), N-(3-dimethylaminopropyl)-N-ethylcarbodiimide hydrochloride (129 mg, 0.67 mmol, 1.50 eq), and diisopropylethylamine (290 mg, 2.25 mmol, 0.4 mL, 5.00 eq), and the reaction mixture was stirred at 25° C. for 7 hours. The mixture was concentrated under reduced pressure, and the resulting residue was purified by prep-thin layer chromatography (EtOAc:CH₃OH=15:1) to afford tert-butyl 3-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]azetidine-1-carboxylate (210 mg, 0.32 mmol, 71% yield) as a white solid. LC/MS (ESI) m/z: 658.3 [M+H]⁺.

### Step 4: Preparation of (2S,4R)-1-[(2S)-2-[[2-(azetidin-3-yloxy)acetyl]amino]-3,3-dimethyl-butanoyl]-4-hydroxy-N-[(1S)-1-[4-(4-methylthiazol-5-yl)phenyl]ethyl]pyrrolidine-2-carboxamide

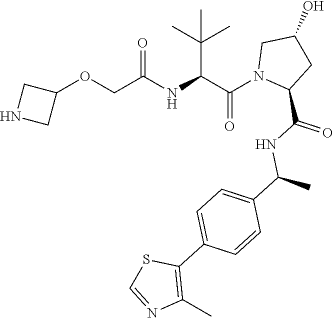 [SMILES: Cc1ncsc1-c1ccc([C@H](C)NC(=O)[C@@H]2C[C@@H](O)CN2C(=O)[C@@H](NC(=O)COC2CNC2)C(C)(C)C)cc1]

To a solution of tert-butyl 3-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]azetidine-1-carboxylate (105 mg, 0.16 mmol, 1.00 eq) in CH₂Cl₂ (2 mL) was added trifluoroacetic acid (770 mg, 6.75 mmol, 0.5 mL, 42.31 eq), and the reaction mixture was stirred at 25° C. for 0.5 hours. The reaction mixture was concentrated under reduced pressure to give (2S,4R)-1-[(2S)-2-[[2-(azetidin-3-yloxy)acetyl]amino]-3,3-dimethyl-butanoyl]-4-hydroxy-N-[(1S)-1-[4-(4-methylthiazol-5-yl)phenyl]ethyl]pyrrolidine-2-carboxamide (105 mg, 0.16 mmol, 98% yield, trifluoroacetic acid salt) as a white solid. LC/MS (ESI) m/z: 558.3 [M+H]⁺.

### Step 5: Preparation of tert-butyl 4-[6-chloro-8-fluoro-2-[(1R)-2-[3-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]azetidin-1-yl]-1-methyl-ethoxy]-7-(3-hydroxy-1-naphthyl)quinazolin-4-yl]piperazine-1-carboxylate

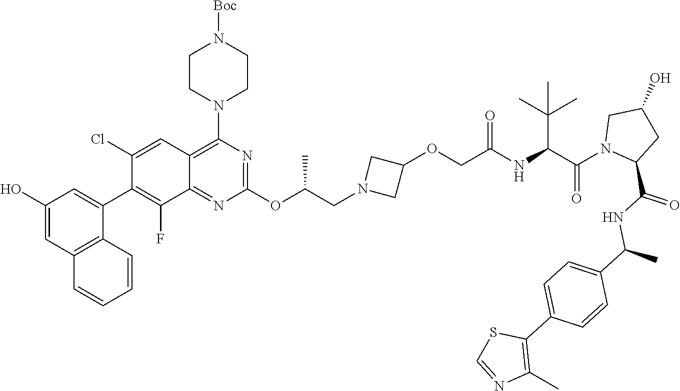 [SMILES: Cc1ncsc1-c1ccc([C@H](C)NC(=O)[C@@H]2C[C@@H](O)CN2C(=O)[C@@H](NC(=O)COC2CN(C[C@@H](C)Oc3nc(N4CCN(C(=O)OC(C)(C)C)CC4)c4cc(Cl)c(-c5cc(O)cc6ccccc56)c(F)c4n3)C2)C(C)(C)C)cc1]

To a solution of tert-butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (100 mg, 0.17 mmol, 1.10 eq) and (2S,4R)-1-[(2S)-2-[[2-(azetidin-3-yloxy)acetyl]amino]-3,3-dimethyl-butanoyl]-4-hydroxy-N-[(1S)-1-[4-(4-methylthiazol-5-yl)phenyl]ethyl]pyrrolidine-2-carboxamide (105 mg, 0.16 mmol, 1.00 eq, trifluoroacetic acid salt) in CH₃OH (0.7 mL) and CH₂Cl₂ (0.7 mL) were added NaOAc (128 mg, 1.56 mmol, 10.00 eq) and acetic acid (93 ug, 1.56 umol, 8.94e-2 uL, 0.01 eq) at 25° C., and the resulting mixture was stirred at 25° C. for 2 hours. 2-Methylpyridine borane (83 mg, 0.78 mmol, 5.00 eq) was then added to the mixture at 0° C., and the reaction mixture was stirred at 40° C. for 10 hours. The mixture was concentrated under reduced pressure, and the resulting residue was purified by prep-thin layer chromatography (CH₂Cl₂:CH₃OH=10:1) to afford tert-butyl 4-[6-chloro-8-fluoro-2-[(1R)-2-[3-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]azetidin-1-yl]-1-methyl-ethoxy]-7-(3-hydroxy-1-naphthyl)quinazolin-4-yl]piperazine-1-carboxylate (80 mg, 0.07 mmol, 45% yield) as a white solid. LC/MS (ESI) m/z: 1122.5 [M+H]⁺.

### Step 6: Preparation of (2S,4R)-1-[(2S)-2-[[2-[1-[(2R)-2-[6-chloro-8-fluoro-7-(3-hydroxy-1-naphthyl)-4-piperazin-1-yl-quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide

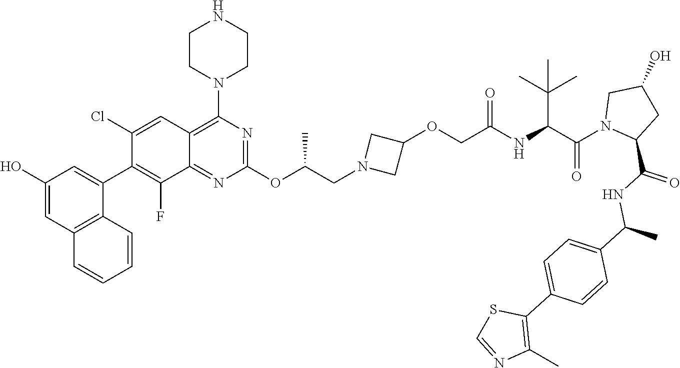 [SMILES: Cc1ncsc1-c1ccc([C@H](C)NC(=O)[C@@H]2C[C@@H](O)CN2C(=O)[C@@H](NC(=O)COC2CN(C[C@@H](C)Oc3nc(N4CCNCC4)c4cc(Cl)c(-c5cc(O)cc6ccccc56)c(F)c4n3)C2)C(C)(C)C)cc1]

To a solution of tert-butyl 4-[6-chloro-8-fluoro-2-[(1R)-2-[3-[2-[[(1S)-1-[(2S,4R)-4-hydroxy-2- [[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]azetidin-1-yl]-1-methyl-ethoxy]-7-(3-hydroxy-1-naphthyl)quinazolin-4-yl]piperazine-1-carboxylate (80 mg, 0.07 mmol, 1.00 eq) in CH₂Cl₂ (2 mL) was added trifluoroacetic acid (770 mg, 6.75 mmol, 0.5 mL, 94.77 eq), and the reaction mixture was stirred at 25° C. for 1 hour. The reaction mixture was concentrated under reduced pressure to give (2S,4R)-1-[(2S)-2-[[2-[1-[(2R)-2-[6-chloro-8-fluoro-7-(3-hydroxy-1-naphthyl)-4-piperazin-1-yl-quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide (80 mg, 0.07 mmol, 98% yield, trifluoroacetic acid salt) as a white solid. LC/MS (ESI) m/z: 1022.5 [M+H]⁺.

### Step 7: Preparation of (2S,4R)-1-[(2S)-2-[[2-[1-[(2R)-2-[6-chloro-8-fluoro-7-(3-hydroxy-1-naphthyl)-4-(4-prop-2-enoylpiperazin-1-yl)quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide

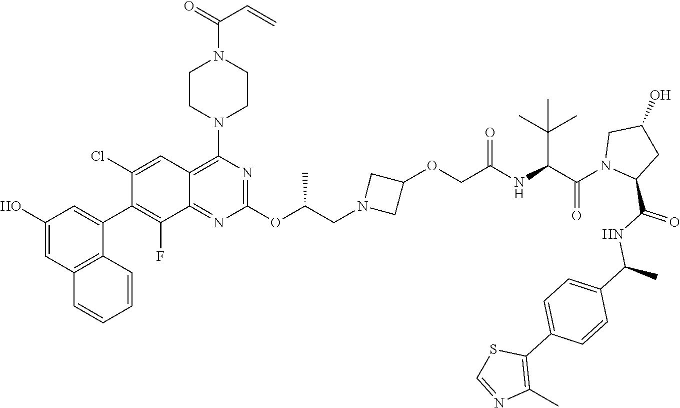 [SMILES: C=CC(=O)N1CCN(c2nc(O[C@H](C)CN3CC(OCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)CC1]

To a solution of (2S,4R)-1-[(2S)-2-[[2-[1-[(2R)-2-[6-chloro-8-fluoro-7-(3-hydroxy-1-naphthyl)-4-piperazin-1-yl-quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide (80 mg, 0.07 mmol, 1.00 eq, trifluoroacetic acid salt) in CH₂Cl₂ (5 mL) at −78° C. were added 2,6-lutidine (75 mg, 0.70 mmol, 10.00 eq) and prop-2-enoyl chloride (6.4 mg, 0.07 mmol, 1.00 eq), and the reaction mixture was stirred at −78° C. for 0.5 hours. Water was then added, and resulting mixture was concentrated. The resulting residue was purified by semi-preparative reverse phase HPLC (25-55% acetonitrile in water (0.225% formic acid)) to afford (2S,4R)-1-[(2S)-2-[[2-[1-[(2R)-2-[6-chloro-8-fluoro-7-(3-hydroxy-1-naphthyl)-4-(4-prop- 2-enoylpiperazin-1-yl)quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide (8.9 mg, 0.008 mmol, 11% yield, 100% purity, formic acid salt) as a white solid after freeze-drying. LC/MS (ESI) m/z: 1076.4 [M+H]⁺. ¹H-NMR (400 MHz, CD₃OD) δ 8.91-8.82 (s, 1H), 8.51 (s, 1H), 8.02 (d, J=5.2 Hz, 1H), 7.75 (d, J=8.0 Hz, 1H), 7.45-7.31 (m, 5H), 7.29-7.15 (m, 3H), 7.08-6.99 (m, 1H), 6.89-6.73 (m, 1H), 6.28 (d, J=16.8 Hz, 1H), 5.81 (d, J=10.4 Hz, 1H), 5.52-5.38 (m, 1H), 4.99 (d, J=7.2 Hz, 1H), 4.62-4.49 (m, 3H), 4.45-4.23 (m, 2H), 4.07-3.97 (m, 5H), 3.96-3.86 (m, 6H), 3.82 (d, J=11.2 Hz, 1H), 3.75-3.69 (m, 1H), 3.62-3.45 (m, 2H), 3.20-2.95 (m, 2H), 2.50-2.41 (m, 3H), 2.39-2.14 (m, 1H), 1.94 (m, 1H), 1.56-1.44 (m, 3H), 1.40 (d, J=6.0 Hz, 3H), 1.00 (s, 9H).

The following compounds were made similarly to (2S,4R)-1-[(2S)-2-[[2-[1-[(2R)-2-[6-chloro-8-fluoro-7-(3-hydroxy-1-naphthyl)-4-(4-prop-2-enoylpiperazin-1-yl)quinazolin-2-yl]oxypropyl]azetidin-3-yl]oxyacetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4R)-1-[(2S)-2-[[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]azetidin-3-yl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

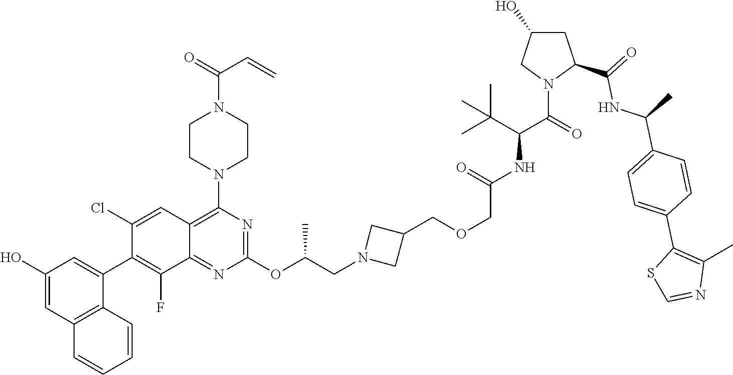 [SMILES: C=CC(=O)N1CCN(c2nc(O[C@H](C)CN3CC(COCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)CC1]

(formic acid salt, white solid). LC/MS (ESI) m/z: 1090.5 [M+H]⁺. ¹H-NMR (400 MHz, CDCl₃) δ 8.92-8.86 (m, 1H), 8.13-8.04 (m, 1H), 7.78 (d, J=8.4 Hz, 1H), 7.47-7.39 (m, 5H), 7.31-7.20 (m, 3H), 7.07-7.03 (m, 1H), 6.89-6.78 (m, 1H), 6.35-6.27 (m, 1H), 5.88-5.80 (m, 1H), 5.55 (s, 1H), 5.05-4.96 (m, 1H), 4.64-4.58 (m, 2H), 4.56 (d, J=7.6 Hz, 1H), 4.38-4.23 (m, 1H), 4.18-4.06 (m, 7H), 3.95 (s, 4H), 3.85 (d, J=10.4 Hz, 1H), 3.78-3.72 (m, 1H), 3.65 (d, J=4.8 Hz, 2H), 3.56-3.47 (m, 2H), 3.17-3.07 (m, 1H), 2.53-2.46 (m, 3H), 2.27-2.17 (m, 1H), 2.00-1.90 (m, 1H), 1.59-1.42 (m, 7H), 1.03 (s, 9H).

### 2. (2S,4R)-1-[(2S)-2-[[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

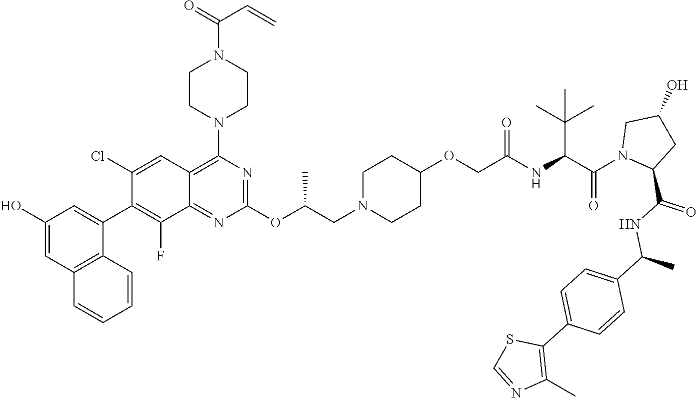 [SMILES: C=CC(=O)N1CCN(c2nc(O[C@H](C)CN3CCC(OCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(formic acid salt, white solid). LC/MS (ESI) m/z: 1104.2 [M+H]⁺. ¹H-NMR (400 MHz, DMSO-d₆) δ 10.36-9.87 (m, 1H), 8.99 (s, 1H), 8.46 (d, J=7.8 Hz, 1H), 8.22 (s, 1H), 8.02 (s, 1H), 7.82 (d, J=8.1 Hz, 1H), 7.47-7.41 (m, 3H), 7.38-7.34 (m, 2H), 7.32-7.27 (m, 2H), 7.25-7.20 (m, 2H), 7.08 (dd, J=2.4, 5.6 Hz, 1H), 6.84 (dd, J=10.4, 16.6 Hz, 1H), 6.19 (dd, J=2.3, 16.7 Hz, 1H), 5.79-5.71 (m, 1H), 5.48-5.35 (m, 1H), 5.23-5.05 (m, 1H), 4.95-4.82 (m, 1H), 4.51 (d, J=9.6 Hz, 1H), 4.47-4.37 (m, 1H), 4.31-4.22 (m, 1H), 3.99-3.89 (m, 6H), 3.86 (br d, J=4.6 Hz, 2H), 3.79 (br d, J=2.5 Hz, 2H), 3.61-3.53 (m, 2H), 2.86-2.71 (m, 2H), 2.67-2.60 (m, 1H), 2.46 (s, 3H), 2.45-2.36 (m, 2H), 2.25-2.12 (m, 2H), 2.09-2.00 (m, 1H), 1.82-1.71 (m, 3H), 1.48-1.40 (m, 1H), 1.40-1.31 (m, 7H), 0.90 (br d, J=4.0 Hz, 9H).

### 3. (2S,4R)-1-[(2S)-2-[[2-[2-[1-[(2R)-2-[6-chloro-8-fluoro-7-(3-hydroxy-1- naphthyl)-4-(4-prop-2-enoylpiperazin-1-yl)quinazolin-2-yl]oxypropyl]-4-piperidyl]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

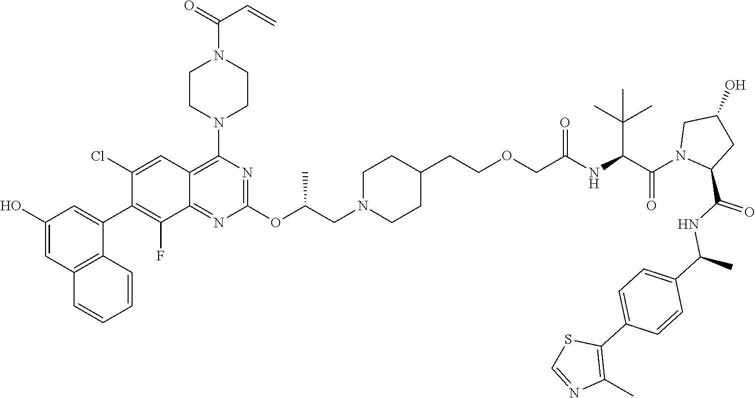 [SMILES: C=CC(=O)N1CCN(c2nc(O[C@H](C)CN3CCC(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(off-white solid). LC/MS (ESI) m/z: 1132.5 [M+H]⁺. ¹H-NMR (400 MHz, DMSO-d₆) δ 10.06-9.90 (m, 1H), 9.02-8.92 (m, 1H), 8.48-8.35 (m, 1H), 8.05-7.96 (m, 1H), 7.86-7.75 (m, 1H), 7.42 (br d, J=7.9 Hz, 2H), 7.35 (br d, J=8.0 Hz, 1H), 7.33-7.14 (m, 3H), 7.12-7.02 (m, 1H), 6.82 (br dd, J=10.8, 16.9 Hz, 1H), 6.18 (br d, J=16.1 Hz, 1H), 5.74 (br d, J=10.3 Hz, 1H), 5.46-5.32 (m, 1H), 5.11 (br d, J=2.4 Hz, 1H), 4.92-4.83 (m, 1H), 4.66-4.47 (m, 1H), 4.43 (br t, J=7.8 Hz, 1H), 4.33-4.18 (m, 1H), 3.92 (br d, J=1.4 Hz, 1H), 4.01-3.67 (m, 1H), 3.62-3.52 (m, 1H), 3.46 (br d, J=1.8 Hz, 2H), 3.25-3.09 (m, 2H), 3.06-2.83 (m, 2H), 2.65-2.55 (m, 1H), 2.47-2.36 (m, 4H), 2.28-1.87 (m, 4H), 1.83-1.69 (m, 1H), 1.67-1.51 (m, 2H), 1.49-1.39 (m, 1H), 1.51-1.39 (m, 1H), 1.38-1.12 (m, 8H), 1.10-0.97 (m, 2H), 0.96-0.44 (m, 9H).

### 4. (2S,4R)-1-[(2S)-2-[[2-[3-[[1-[(2R)-2-[6-chloro-8-fluoro-7-(3-hydroxy-1- naphthyl)-4-(4-prop-2-enoylpiperazin-1-yl)quinazolin-2-yl]oxypropyl]-4-piperidylmethyl-methyl-amino]cyclobutoxy]acetyl]amino]-3,3-dimethyl-butanoyl]-4-hydroxy-N-[(1S)-1-[4-(4-methylthiazol-5-yl) phenyl]ethyl]pyrrolidine-2-carboxamide

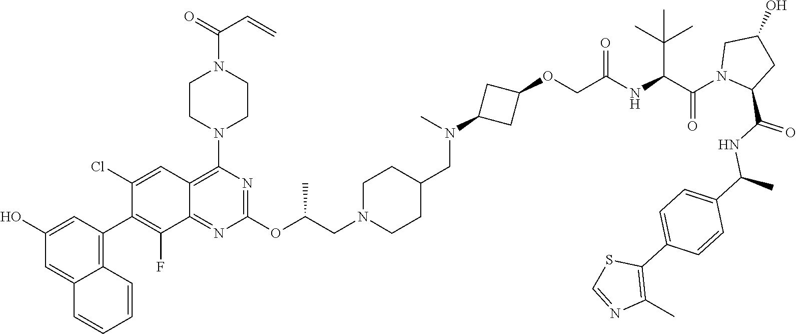 [SMILES: C=CC(=O)N1CCN(c2nc(O[C@H](C)CN3CCC(CN(C)[C@H]4C[C@@H](OCC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1]

(formic acid salt, white solid). LC/MS (ESI) m/z: 1201.6 [M+H]⁺. ¹H-NMR (400 MHz, DMSO-d₆) δ 8.98 (s, 1H), 8.51-8.39 (m, 1H), 8.20 (s, 1H), 8.25-8.16 (m, 1H), 8.00 (s, 1H), 7.81 (d, J=8.4 Hz, 1H), 7.47-7.40 (m, 3H), 7.39-7.34 (m, 2H), 7.34-7.27 (m, 2H), 7.26-7.18 (m, 2H), 7.07 (d, J=1.1 Hz, 1H), 6.84 (dd, J=10.4, 16.6 Hz, 1H), 6.18 (dd, J=2.2, 16.8 Hz, 1H), 5.79-5.72 (m, 1H), 5.46-5.34 (m, 1H), 5.31-4.80 (m, 2H), 4.52 (d, J=9.7 Hz, 1H), 4.48-4.38 (m, 1H), 4.31-4.25 (m, 1H), 3.92 (br s, 4H), 3.85 (br s, 2H), 3.81 (s, 2H), 3.78 (br s, 2H), 3.76-3.69 (m, 2H), 3.61-3.53 (m, 4H), 2.95-2.84 (m, 2H), 2.66-2.56 (m, 1H), 2.46 (s, 3H), 2.42-2.35 (m, 3H), 2.30-2.23 (m, 1H), 2.09-1.99 (m, 2H), 1.96 (br d, J=3.5 Hz, 4H), 1.93-1.89 (m, 2H), 1.81-1.73 (m, 1H), 1.65-1.54 (m, 4H), 1.37 (d, J=7.0 Hz, 3H), 1.31 (br d, J=6.0 Hz, 3H), 0.92 (s, 9H).

### Exemplary Synthesis of tert-butyl 4-[6-chloro-8-fluoro-7-(3-hydroxy-1-naphthyl)-2-(2-oxoethoxy)quinazolin-4-yl]piperazine-1-carboxylate

### Step 1: Preparation of tert-butyl 4-[7-bromo-6-chloro-2-(2,2-dimethoxyethoxy)-8-fluoro-quinazolin-4-yl]piperazine-1-carboxylate

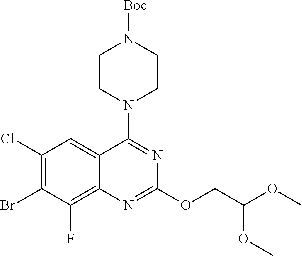 [SMILES: COC(COc1nc(N2CCN(C(=O)OC(C)(C)C)CC2)c2cc(Cl)c(Br)c(F)c2n1)OC]

To a solution of tert-butyl 4-(7-bromo-2,6-dichloro-8-fluoro-quinazolin-4-yl)piperazine-1-carboxylate (1.50 g, 3.12 mmol, 1.00 eq) and 2,2-dimethoxyethanol (663 mg, 6.25 mmol, 2.00 eq) in CH₃CN (15 mL) was added Cs₂CO₃ (1.32 g, 4.06 mmol, 1.30 eq), and the reaction mixture was stirred at 45° C. for 7 hours. The mixture was filtered, and the filtrate was concentrated. The resulting residue was purified by silica gel chromatography (3-7% EtOAc in petroleum ether to afford tert-butyl 4-[7-bromo-6-chloro-2-(2,2-dimethoxyethoxy)-8-fluoro-quinazolin-4-yl]piperazine-1-carboxylate (1.30 g, 2.36 mmol, 75% yield) as a light yellow solid. LC/MS (ESI) m/z: 551.3 [M+H]⁺. ¹H-NMR (400 MHz, CDCl₃) δ 7.73-7.70 (d, J=2.0 Hz, 1H), 4.87-4.81 (t, J=5.2 Hz, 1H), 4.50 (d, J=5.2 Hz, 2H), 3.84-3.73 (m, 4H), 3.68-3.61 (m, 4H), 3.50-3.45 (s, 6H), 1.50 (s, 9H).

### Step 2: Preparation of tert-butyl 4-[6-chloro-2-(2,2-dimethoxyethoxy)-8-fluoro-7-(3-hydroxy-1-naphthyl)quinazolin-4-yl]piperazine-1-carboxylate

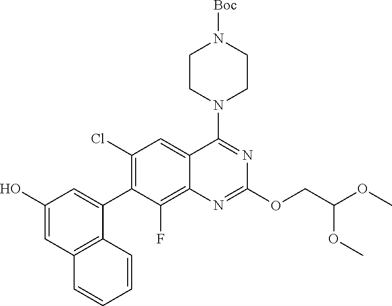 [SMILES: COC(COc1nc(N2CCN(C(=O)OC(C)(C)C)CC2)c2cc(Cl)c(-c3cc(O)cc4ccccc34)c(F)c2n1)OC]

To a solution of tert-butyl 4-[7-bromo-6-chloro-2-(2,2-dimethoxyethoxy)-8-fluoro-quinazolin-4-yl]piperazine-1-carboxylate (1.30 g, 2.36 mmol, 1.00 eq) and 4-(4,4,5,5-tetramethyl-1,3,2-dioxaborolan-2-yl)naphthalen-2-ol (640 mg, 2.37 mmol, 1.00 eq) in THF (12 mL) were added K₃PO₄ (1.5 M, 4.7 mL, 2.98 eq) and methanesulfonato(2-dicyclohexylphosphino-2,4,6-tri-i-propyl-1,1-biphenyl)(2-amino-1,1-biphenyl-2-yl)palladium(ii) (200 mg, 0.23 mmol, 0.10 eq), and the reaction mixture was stirred at 45° C. for 5 hours. The mixture was filtered, and the filtrate was concentrated. The resulting residue was purified by silica gel chromatography (7-50% EtOAc in petroleum ether) to afford tert-butyl 4-[6-chloro-2-(2,2-dimethoxyethoxy)-8-fluoro-7-(3-hydroxy-1-naphthyl)quinazolin-4-yl]piperazine-1-carboxylate (1.30 g, 2.12 mmol, 89% yield) as a light yellow solid. LC/MS (ESI) m/z: 613.3 [M+H]⁺. ¹H-NMR (400 MHz, CDCl₃) δ 7.81-7.76 (m, 2H), 7.46 (m, 1H), 7.34-7.28 (m, 3H), 7.10 (d, J=2.8 Hz, 1H), 5.45 (s, 1H), 4.86 (t, J=5.6 Hz, 1H), 4.51 (d, J=5.2 Hz, 2H), 3.85 (m, 4H), 3.69 (m, 4H), 3.46 (d, J=1.2 Hz, 6H), 1.52 (s, 9H).

### Step 3: Preparation of 2-[6-chloro-8-fluoro-7-(3-hydroxy-1-naphthyl)-4-piperazin-1-yl-quinazolin-2-yl]oxyacetaldehyde

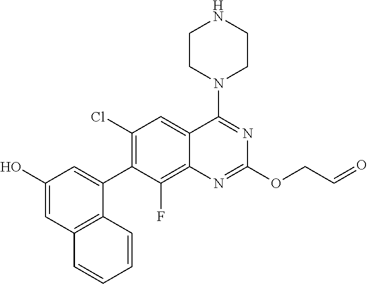 [SMILES: O=CCOc1nc(N2CCNCC2)c2cc(Cl)c(-c3cc(O)cc4ccccc34)c(F)c2n1]

To a solution of tert-butyl 4-[6-chloro-2-(2,2-dimethoxyethoxy)-8-fluoro-7-(3-hydroxy-1-naphthyl)quinazolin-4-yl]piperazine-1-carboxylate (1.10 g, 1.79 mmol, 1.00 eq) in dioxane (8 mL) was added aqueous HCl (12N, 2 mL, 13.38 eq), and the reaction mixture was stirred at 25° C. for 1 hour. The reaction mixture was concentrated under reduced pressure to give 2-[6-chloro-8-fluoro-7-(3-hydroxy-1-naphthyl)-4-piperazin-1-yl-quinazolin-2-yl]oxyacetaldehyde (900 mg, 1.79 mmol, 100% yield, hydrochloride salt) as a yellow solid. LC/MS (ESI) m/z: 467.3 [M+H]⁺.

### Step 4: Preparation of tert-butyl 4-[6-chloro-8-fluoro-7-(3-hydroxy-1-naphthyl)-2-(2-oxoethoxy)quinazolin-4-yl]piperazine-1-carboxylate

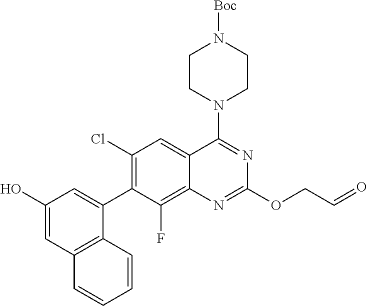 [SMILES: CC(C)(C)OC(=O)N1CCN(c2nc(OCC=O)nc3c(F)c(-c4cc(O)cc5ccccc45)c(Cl)cc23)CC1]

To a solution of 2-[6-chloro-8-fluoro-7-(3-hydroxy-1-naphthyl)-4-piperazin-1-yl-quinazolin-2-yl]oxyacetaldehyde (900 mg, 1.79 mmol, 1.00 eq, hydrochloride) in THF (10 mL) and water (4 mL) were added NaHCO₃ (810 mg, 9.64 mmol, 5.38 eq) and di-tert-butyl dicarbonate (841 mg, 3.86 mmol, 0.9 mL, 2.15 eq), and the reaction mixture was stirred at 25° C. for 1 hour. The reaction mixture was concentrated, and the resulting residue was diluted with water (5 mL) and extracted with CH₂Cl₂ (3×5 mL). The combined organic layers were concentrated, and the resulting residue was purified by silica gel chromatography (7-50% EtOAc in petroleum ether) to afford tert-butyl 4-[6-chloro-8-fluoro-7-(3-hydroxy-1-naphthyl)-2-(2-oxoethoxy)quinazolin-4-yl]piperazine-1-carboxylate (600 mg, 1.06 mmol, 59% yield) as a yellow solid. LC/MS (ESI) m/z: 567.4 [M+H]⁺.

### Exemplary Synthesis of (2S,4R)-1-[(2S)-2-[[2-[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-[6-chloro-8-fluoro-2-[2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]ethoxy]-7-(3-hydroxy-1-naphthyl)quinazolin-4-yl]piperazine-1-carboxylate

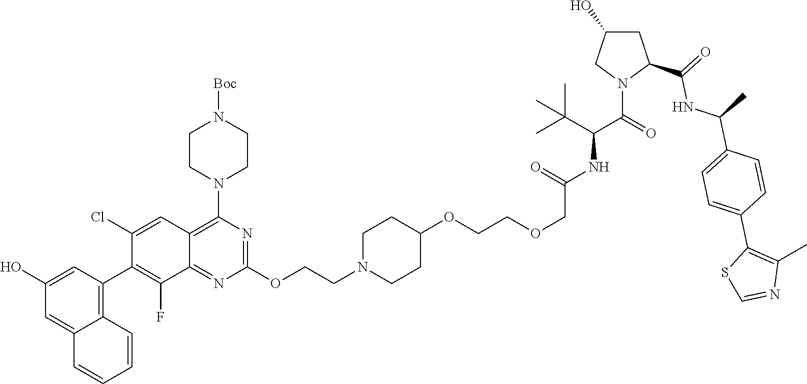 [SMILES: Cc1ncsc1-c1ccc([C@H](C)NC(=O)[C@@H]2C[C@@H](O)CN2C(=O)[C@@H](NC(=O)COCCOC2CCN(CCOc3nc(N4CCN(C(=O)OC(C)(C)C)CC4)c4cc(Cl)c(-c5cc(O)cc6ccccc56)c(F)c4n3)CC2)C(C)(C)C)cc1]

To a solution of (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide; hydrochloride (105 mg, 0.15 mmol, 1.00 eq, hydrochloride) in dichloroethane (1.5 mL) was added NaOAc (123 mg, 1.49 mmol, 10.00 eq) at 25° C., and the resulting mixture was stirred at 25° C. for 0.5 hours. Acetic acid (0.09 mg, 0.001 mmol, 0.01 eq) and tert-butyl 4-[6-chloro-8-fluoro-7-(3-hydroxy-1-naphthyl)-2-(2-oxoethoxy)quinazolin-4-yl]piperazine-1-carboxylate (93 mg, 0.16 umol, 1.10 eq) were then added at 0° C., and the mixture was stirred at 0° C. for 0.5 hours. NaBH₃CN (18 mg, 0.30 mmol, 2.00 eq) was then added, and the reaction mixture was stirred at 25° C. for 12 hours. The reaction mixture was concentrated, and the resulting residue was purified by prep-thin layer chromatography (CH₂Cl₂:CH₃OH=10:1) to afford tert-butyl 4-[6-chloro-8-fluoro-2-[2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]ethoxy]-7-(3-hydroxy-1-naphthyl)quinazolin-4-yl]piperazine-1-carboxylate (80 mg, 0.06 mmol, 45% yield) as a white solid. LC/MS (ESI) m/z: 1180.9 [M+H]⁺.

### Step 2: Preparation of (2S,4R)-1-[(2S)-2-[[2-[2-[[1-[2-[6-chloro-8-fluoro-7-(3-hydroxy-1-naphthyl)-4-piperazin-1-yl-quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

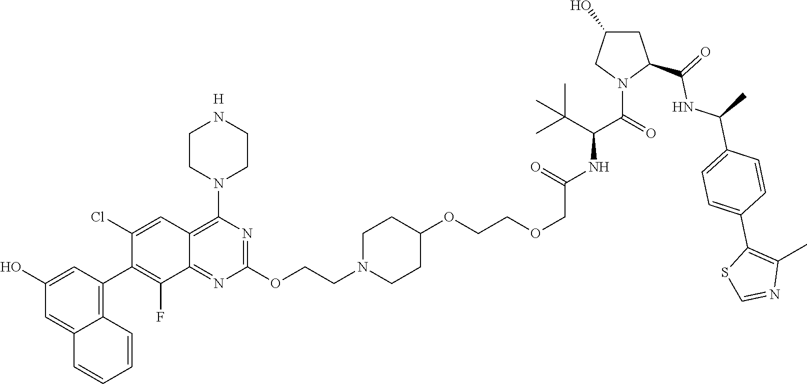

To a solution of tert-butyl 4-[6-chloro-8-fluoro-2-[2-[4-[2-[2-[[(1S)-1-[(2S,4R)-4-hydroxy-2- [[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]ethoxy]-7-(3-hydroxy-1-naphthyl)quinazolin-4-yl]piperazine-1-carboxylate (80 mg, 0.06 mmol, 1.00 eq) in CH₂Cl₂ (2 mL) was HCl (4N in dioxane, 1.00 mL, 59.04 eq), and the reaction mixture was stirred at 25° C. for 1 hour. The reaction mixture was concentrated under reduced pressure to give (2S,4R)-1-[(2S)-2-[[2-[2-[[1-[2-[6-chloro-8-fluoro-7-(3-hydroxy-1-naphthyl)-4-piperazin-1-yl-quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (78 mg, 67.61 umol, 100% yield, hydrogen chloride salts) as a white solid.

### Step 3: Preparation of (2S,4R)-1-[(2S)-2-[[2-[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

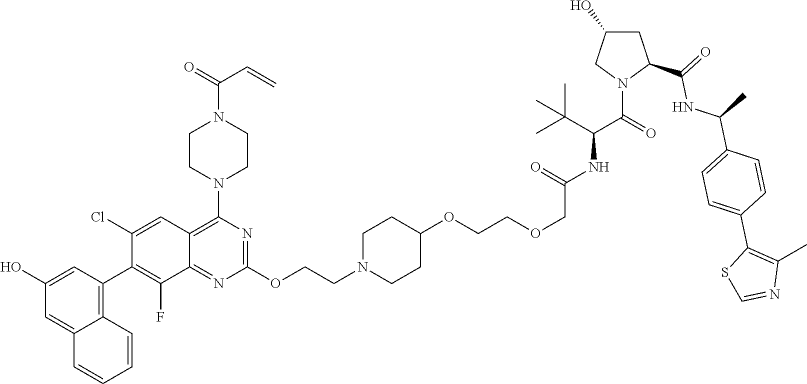 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To a solution of (2S,4R)-1-[(2S)-2-[[2-[2-[[1-[2-[6-chloro-8-fluoro-7-(3-hydroxy-1-naphthyl)- 4-piperazin-1-yl-quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (78 mg, 0.06 mmol, 1.00 eq, two hydrogen chloride) in CH₂Cl₂ (5 mL) at −78° C. were added 2,6-lutidine (72 mg, 0.6 mmol, 0.1 mL, 10.00 eq) and prop-2-enoyl chloride (6 mg, 0.06 mmol, 1.00 eq), and the reaction mixture was stirred at −78° C. for 0.5 hours. The reaction mixture was quenched by addition of CH₃OH at −78° C., and the resulting mixture was concentrated. The resulting residue was purified by semi-preparative reverse phase HPLC (25-58% acetonitrile in water (0.1% formic acid)) to afford (2S,4R)-1-[(2S)-2-[[2-[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (11.7 mg, 0.01 mmol, 15% yield, 98% purity) as a white solid after freeze-drying. LC/MS (ESI) m/z: 1134.5 [M+H]⁺. ¹H-NMR (400 MHz, CD₃OD) δ 8.90-8.79 (m, 1H), 7.99 (s, 1H), 7.73 (d, J=7.6 Hz, 1H), 7.45-7.31 (m, 5H), 7.26 (d, J=2.0 Hz, 1H), 7.23-7.12 (m, 2H), 7.04 (d, J=2.0 Hz, 1H), 6.78 (m, 1H), 6.28 (d, J=16.4 Hz, 1H), 5.80 (d, J=10.4 Hz, 1H), 5.02-4.95 (m, 1H), 4.69 (s, 2H), 4.60-4.52 (m, 1H), 4.42 (s, 1H), 4.10-3.44 (m, 18H), 3.16 (s, 4H), 3.04-2.72 (m, 2H), 2.44 (s, 3H), 2.25-2.14 (m, 1H), 2.07-1.73 (m, 5H), 1.60-1.39 (m, 3H), 1.11-0.94 (s, 9H).

The following compound can be prepared in an analogous manner to (2S,4R)-1-[(2S)-2-[[2-[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4R)-1-[(2S)-2-[[2-[2-[2-[[1-[2-[6-chloro-8-fluoro-7-(3-hydroxy-1- naphthyl)-4-(4-prop-2-enoylpiperazin-1-yl)quinazolin-2-yl]oxyethyl]-4-piperidyl]oxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

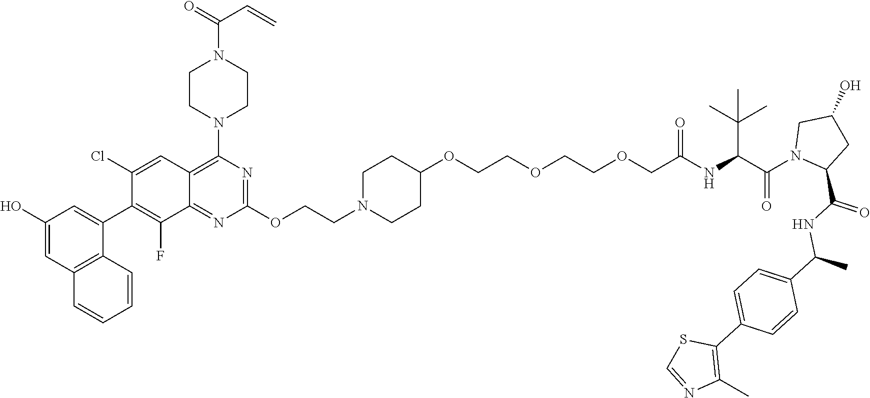 [SMILES: C=CC(=O)N1CCN(c2nc(OCCN3CCC(OCCOCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(formic acid salt; white solid). LC/MS (ESI) m/z: 1178.2[M+H]⁺. ¹H-NMR (400 MHz, CD₃OD) δ 8.90-8.80 (m, 1H), 8.52 (d, J=1.6 Hz, 1H), 8.04 (s, 1H), 7.75 (d, J=8.4 Hz, 1H), 7.48-7.31 (m, 5H), 7.30-7.12 (m, 3H), 7.07-7.01 (m, 1H), 6.82 (dd, J=10.8, 16.8 Hz, 1H), 6.29 (dd, J=1.6, 16.8 Hz, 1H), 5.82 (dd, J=1.6, 10.8 Hz, 1H), 4.99 (d, J=7.2 Hz, 1H), 4.71 (s, 2H), 4.58-4.52 (m, 2H), 4.46-4.29 (m, 1H), 4.05 (d, J=1.6 Hz, 6H), 3.96-3.80 (m, 5H), 3.79-3.41 (m, 11H), 3.30-3.09 (m, 4H), 2.90-2.77 (m, 1H), 2.48-2.42 (m, 3H), 2.20 (dd, J=7.6, 13.6 Hz, 1H), 2.04-1.67 (m, 5H), 1.60-1.39 (m, 3H), 1.07-0.98 (m, 9H).

### Exemplary Synthesis of (2S,4R)-1-[(2S)-2-[[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl4-[6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)quinazolin-4-yl]piperazine-1-carboxylate

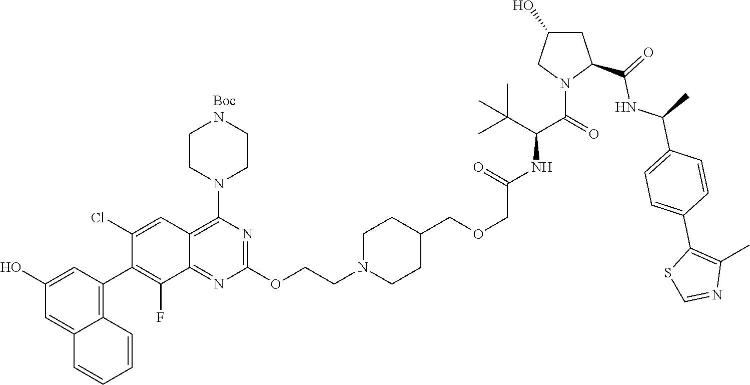 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCNCC4)c4cc(Cl)c(-c5cc(O)cc6ccccc56)c(F)c4n3)CC2)C(C)(C)C)cc1]

To a solution of (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (90 mg, 0.14 mmol, 1.00 eq, hydrochloride salt) and tert-butyl 4-[6-chloro-8-fluoro-7-(3-hydroxy-1-naphthyl)-2-(2-oxoethoxy)quinazolin-4-yl]piperazine-1-carboxylate (88 mg, 0.16 mmol, 1.10 eq) in 1,2-dichloroethane (1.5 mL) and CH₃OH (1 mL) were added acetic acid (26 mg, 0.42 mmol, 3.00 eq), NaOAc (35 mg, 0.42 mmol, 3.00 eq), and NaBH₃CN (20 mg, 0.28 mmol, 2.00 eq), and the reaction mixture was stirred at 25° C. for 12 hours. The solution was concentrated, and the remaining material was purified by thin layer chromatography (CH₂Cl₂/CH₃OH=10/1) to afford tert-butyl4-[6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)quinazolin-4-yl]piperazine-1-carboxylate (40 mg, 0.03 mmol, 25% yield) as a yellow oil. LC/MS (ESI) m/z: 1151.2 [M+H]⁺.

### Step 2: Preparation of (2S,4R)-1-[(2S)-2-[[2-[[1-[2-[6-chloro-8-fluoro-7-(3-hydroxy-1-naphthyl)-4-piperazin-1-yl-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

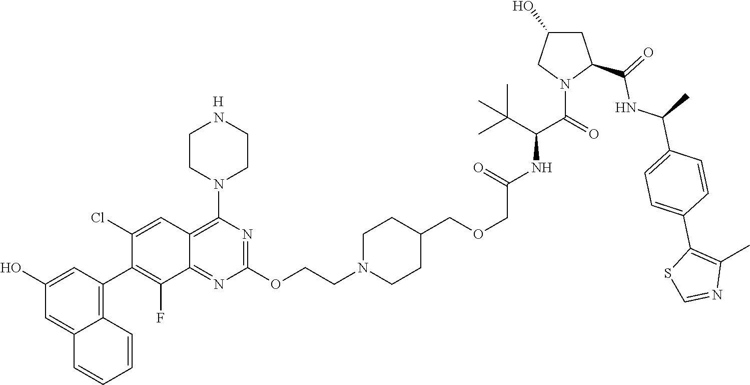

To a solution of tert-butyl 4-[6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)quinazolin-4-yl]piperazine-1-carboxylate (40 mg, 0.03 mmol, 1.00 eq) in CH₂Cl₂ (2 mL) was added HCl (4N in dioxane, 2 mL), and the reaction mixture was stirred at 25° C. for 10 minutes. The solution was concentrated to afford (2S,4R)-1-[(2S)-2-[[2-[[1-[2-[6-chloro-8-fluoro-7-(3-hydroxy-1-naphthyl)-4-piperazin-1-yl-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (25 mg, 0.02 mmol, 66% yield, hydrochloride salt) as a yellow solid. LC/MS (ESI) m/z: 1050.3 [M+H]⁺.

### Step 3: Preparation of (2S,4R)-1-[(2S)-2-[[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

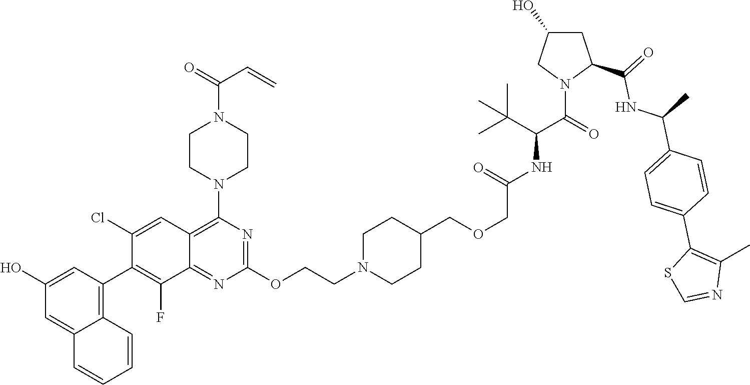 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To a solution of (2S,4R)-1-[(2S)-2-[[2-[[1-[2-[6-chloro-8-fluoro-7-(3-hydroxy-1-naphthyl)-4-piperazin-1-yl-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (25 mg, 0.02 mmol, 1.00 eq, hydrochloride salt) in CH₂Cl₂ (10 mL) at −75° C. were added 2,6-dimethylpyridine (25 mg, 0.23 mmol, 10.00 eq) and prop-2-enoyl chloride (2 mg, 0.02 mmol, 1.00 eq), and the reaction mixture was stirred at −75° C. for 0.5 hour. The solution was concentrated, and the remaining material was purified by prep-HPLC (formic acid condition) to afford (2S,4R)-1-[(2S)-2-[[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (5.9 mg, 21% yield, 93% purity, formic acid salt) as a white solid. LC/MS (ESI) m/z: 1104.5 [M+H]⁺. ¹H-NMR (400 MHz, CD₃OD) δ 8.89-8.83 (m, 1H), 8.52 (s, 1H), 8.03 (s, 1H), 7.78-7.70 (m, 1H), 7.45-7.33 (m, 5H), 7.29-7.15 (m, 3H), 7.04 (d, J=2.4 Hz, 1H), 6.81 (dd, J=10.8, 16.8 Hz, 1H), 6.28 (dd, J=1.6, 16.8 Hz, 1H), 5.85-5.76 (m, 1H), 4.97 (d, J=7.2 Hz, 1H), 4.70 (d, J=5.6 Hz, 2H), 4.58-4.50 (m, 2H), 4.43 (s, 1H), 4.08-3.68 (m, 12H), 3.47-3.32 (m, 4H), 3.16 (s, 2H), 2.72-2.49 (m, 2H), 2.46 (s, 3H), 2.40-2.16 (m, 1H), 2.04-1.73 (m, 4H), 1.62-1.37 (m, 5H), 1.08-0.95 (m, 9H).

The following atropisomers can be separated by SFC.

### 1. (2S,4R)-1-[(2S)-2-[[2-[[1-[2-[6-chloro-8-fluoro-7-(3-hydroxy-1-naphthyl)-4-(4- prop-2-enoylpiperazin-1-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

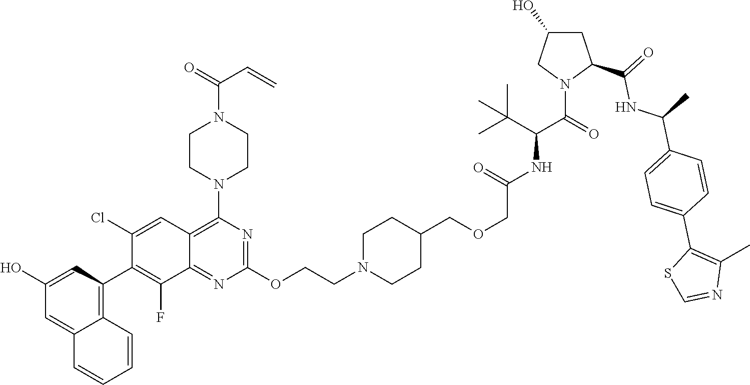

(formic acid salt, white solid). LC/MS (ESI) m/z: 1104.6 [M+1]⁺. ¹H-NMR (400 MHz, CD₃OD) δ 8.88 (s, 1H), 8.65-8.44 (m, 1H), 8.05 (d, J=1.6 Hz, 1H), 7.82-7.67 (m, 1H), 7.45-7.37 (m, 5H), 7.31-7.13 (m, 3H), 7.04 (d, J=2.4 Hz, 1H), 6.83 (dd, J=10.4, 16.8 Hz, 1H), 6.29 (dd, J=1.6, 16.8 Hz, 1H), 5.82 (dd, J=1.6, 10.4 Hz, 1H), 5.06-4.92 (m, 3H), 4.81-4.65 (m, 2H), 4.64-4.49 (m, 1H), 4.48-4.35 (m, 1H), 4.05 (s, 4H), 3.99 (s, 1H), 3.97-3.90 (m, 5H), 3.88-3.80 (m, 1H), 3.79-3.69 (m, 1H), 3.43 (d, J=5.6 Hz, 4H), 3.22-3.06 (m, 2H), 2.48-2.44 (m, 3H), 2.35-2.09 (m, 1H), 2.04-1.74 (m, 4H), 1.47 (d, J=7.2 Hz, 5H), 1.05-1.01 (m, 9H).

### 2. (2S,4R)-1-[(2S)-2-[[2-[[1-[2-[6-chloro-8-fluoro-7-(3-hydroxy-1-naphthyl)-4-(4- prop-2-enoylpiperazin-1-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

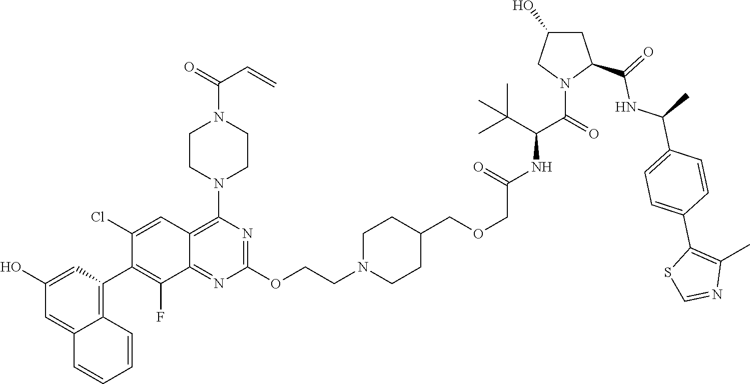

(formic acid salt, white solid). ¹H-NMR (400 MHz, CD₃OD) δ 8.92-8.81 (m, 1H), 8.52 (s, 1H), 8.05 (s, 1H), 7.75 (d, J=8.4 Hz, 1H), 7.49-7.32 (m, 5H), 7.30-7.13 (m, 3H), 7.03 (d, J=2.0 Hz, 1H), 6.82 (dd, J=10.4, 16.8 Hz, 1H), 6.29 (dd, J=1.6, 16.8 Hz, 1H), 5.81 (dd, J=1.6, 10.4 Hz, 1H), 5.04-4.92 (m, 2H), 4.79-4.64 (m, 4H), 4.62-4.49 (m, 1H), 4.48-4.30 (m, 1H), 4.14-3.70 (m, 12H), 3.61-3.36 (m, 5H), 3.23-3.11 (m, 2H), 2.62 (s, 2H), 2.47 (s, 3H), 2.21 (dd, J=8.0, 13.2 Hz, 1H), 2.07-1.75 (m, 4H), 1.62-1.40 (m, 5H), 1.10-0.93 (m, 9H).

The following compounds can be prepared in an analogous manner to (2S,4R)-1-[(2S)-2-[[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4S)-1-((2S)-2-(2-((1-(2-((4-(4-acryloylpiperazin-1-yl)-6-chloro-8-fluoro-7-(3-hydroxynaphthalen-1-yl)quinazolin-2-yl)oxy)ethyl)piperidin-4-yl)methoxy)acetamido)-3,3-dimethylbutanoyl)-4-hydroxy-N—((S)-1-(4-(4-methylthiazol-5-yl)phenyl)ethyl)pyrrolidine-2-carboxamide

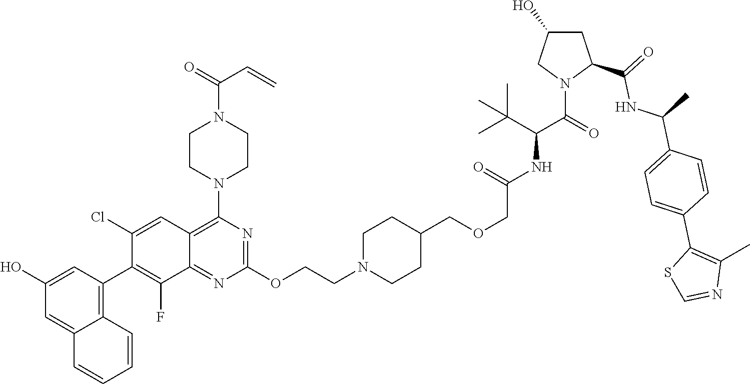 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(white solid). LC/MS (ESI) m/z: 1104.4 [M+1]⁺. ¹H-NMR (400 MHz, DMSO-d₆) δ 10.0 (s, 1H), 8.98 (s, 1H), 8.39-8.37 (m, 1H), 8.13 (s, 1H), 8.04-8.03 (m, 1H), 7.50-7.10 (m, 9H), 7.07-7.06 (m, 1H), 6.83-6.82 (m, 1H), 6.24-6.19 (m, 1H), 5.76-5.73 (m, 1H), 5.32-5.30 (m, 1H), 4.90-4.80 (m, 1H), 4.60-4.20 (m, 4H), 3.94-3.83 (m, 12H), 3.35-3.29 (m, 12H), 2.56 (s, 3H), 1.64-1.60 (m, 4H), 1.36-1.23 (m, 3H), 0.93-0.92 (m, 9H).

### 2. (2S,4R)-1-[(2S)-2-[[2-[2-[4-[2-[6-chloro-8-fluoro-7-(3-hydroxy-1-naphthyl)-4- (4-prop-2-enoylpiperazin-1-yl)quinazolin-2-yl]oxyethyl]piperazin-1-yl]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

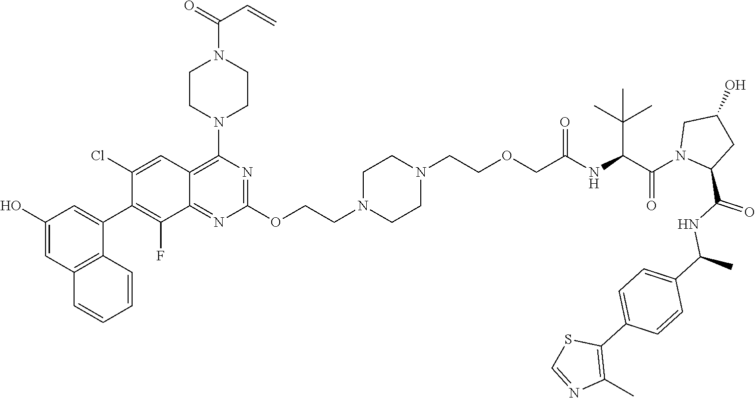 [SMILES: C=CC(=O)N1CCN(c2nc(OCCN3CCN(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(formic acid salt, white solid). LC/MS (ESI) m/z: 1119.5 [M+H]⁺. ¹H-NMR (400 MHz, CDCl₃) δ 8.98-8.78 (m, 1H), 8.06 (s, 1H), 7.76 (d, J=8.4 Hz, 1H), 7.46-7.38 (m, 5H), 7.31-7.19 (m, 3H), 7.05 (d, J=2.4 Hz, 1H), 6.84 (d, J=16.8 Hz, 1H), 6.31 (d, J=18.4 Hz, 1H), 5.83 (d, J=12.4 Hz, 1H), 5.00 (d, J=6.4 Hz, 1H), 4.68 (s, 1H), 4.56 (s, 1H), 4.44 (s, 1H), 4.09-3.92 (m, 10H), 3.82-3.71 (m, 6H), 3.37 (s, 2H), 3.13-2.87 (m, 11H), 2.47 (d, J=3.2 Hz, 3H), 2.18 (s, 1H), 1.89 (d, J=6.8 Hz, 2H), 1.50 (d, J=7.2 Hz, 3H), 1.07-1.02 (m, 9H).

### 3. (2S,4R)-1-[(2S)-2-[[2-[2-[1-[2-[6-chloro-8-fluoro-7-(3-hydroxy-1-naphthyl)-4- (4-prop-2-enoylpiperazin-1-yl)quinazolin-2-yl]oxyethyl]-4-piperidyl]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

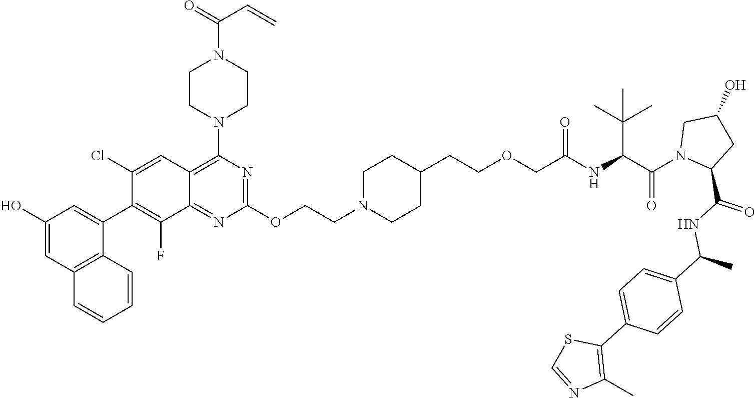 [SMILES: C=CC(=O)N1CCN(c2nc(OCCN3CCC(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(formic acid salt, white solid). LC/MS (ESI) m/z: 1118.5 [M+H]⁺. ¹H-NMR (400 MHz, CD₃OD) δ 8.89-8.82 (m, 1H), 8.02 (d, J=1.2 Hz, 1H), 7.75 (d, J=8.4 Hz, 1H), 7.47-7.33 (m, 5H), 7.26 (d, J=2.0 Hz, 1H), 7.24-7.13 (m, 2H), 7.04 (s, 1H), 6.86-6.75 (m, 1H), 6.28 (d, J=16.8 Hz, 1H), 5.84 (s, 1H), 5.03-4.94 (m, 1H), 4.76-4.68 (m, 2H), 4.61-4.50 (m, 1H), 4.46-4.31 (m, 1H), 4.14-3.69 (m, 12H), 3.67-3.47 (m, 3H), 3.41 (s, 2H), 3.23 (s, 2H), 2.85-2.59 (m, 2H), 2.45 (d, J=2.0 Hz, 3H), 2.21 (m, 1H), 2.02-1.84 (m, 3H), 1.76-1.35 (m, 8H), 1.08-0.94 (s, 9H).

### Exemplary Synthesis of 2S,4R)-1-[(2S)-2-[[2-[[1-[(2R)-2-[6-chloro-8-fluoro-4-[4-(2-fluoroprop-2-enoyl)piperazin-1-yl]-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

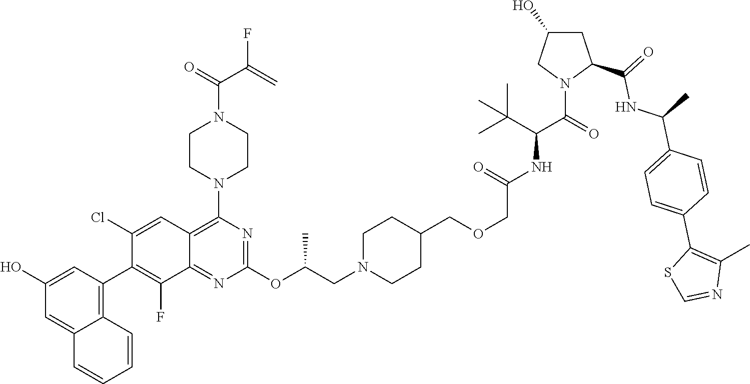 [SMILES: C=C(F)C(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

A mixture of (2S,4R)-1-[(2S)-2-[[2-[[1-[(2R)-2-[6-chloro-8-fluoro-7-(3-hydroxy-1-naphthyl)-4-piperazin-1-yl-quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (56 mg, 0.051 mmol, 1 eq, hydrochloride), 2-fluoroprop-2-enoic acid (5 mg, 0.051 mmol, 1 eq), triethylamine (10 mg, 0.010 mmol, 2 eq), and HATU (29 mg, 0.076 mmol, 1.5 eq) in DMF (1 mL) was degassed and purged with nitrogen (3×), and the reaction mixture was stirred at 25° C. for 0.5 h under nitrogen atmosphere. Water (0.01 mL) was added followed by EtOAc (10 mL), and the resulting mixture was extracted with water (3×5 mL). The combined organic extract was dried over anhydrous Na₂SO₄, filtered, and concentrated. The resulting residue was purified by semi-preparative reverse phase HPLC (28%-58% CH₃CN in water (0.225% formic acid)) to afford (2S,4R)-1-[(2S)-2-[[2-[[1-[(2R)-2-[6-chloro-8-fluoro-4-[4-(2-fluoroprop-2-enoyl)piperazin-1-yl]-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (26.4 mg, 0.016 mmol, 32% yield, 94% purity, formic acid) as a white solid. LC/MS (ESI) m/z: 1136.3 [M+H]⁺. ¹H-NMR (400 MHz, DMSO-d₆) δ 10.08-9.96 (m, 1H), 8.99 (s, 1H), 8.44 (d, J=7.9 Hz, 1H), 8.16 (s, 1H), 8.01 (s, 1H), 7.81 (d, J=8.4 Hz, 1H), 7.47-7.41 (m, 3H), 7.36 (dd, J=1.3, 8.2 Hz, 2H), 7.31-7.24 (m, 2H), 7.24-7.15 (m, 2H), 7.07 (dd, J=2.3, 4.1 Hz, 1H), 5.45-5.33 (m, 2H), 5.25-5.11 (m, 1H), 4.95-4.81 (m, 1H), 4.53 (d, J=9.5 Hz, 1H), 4.48-4.40 (m, 1H), 4.32-4.22 (m, 1H), 3.96 (br d, J=1.0 Hz, 4H), 3.89 (s, 2H), 3.87-3.74 (m, 4H), 3.61-3.53 (m, 2H), 3.27-3.25 (m, 2H), 3.02-2.90 (m, 2H), 2.66-2.57 (m, 1H), 2.46 (s, 5H), 2.10-1.96 (m, 3H), 1.80-1.72 (m, 1H), 1.65-1.47 (m, 3H), 1.39-1.29 (m, 6H), 1.17-1.01 (m, 2H), 0.91 (s, 9H).

### Exemplary Synthesis of 1-[(2R)-2-[6-chloro-8-fluoro-7-(3-hydroxy-1-naphthyl)-4-(4-prop-2-enoylpiperazin-1-yl)quinazolin-2-yl]oxypropyl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

### Step 1: Preparation of tert-butyl 4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]piperidine-1-carboxylate

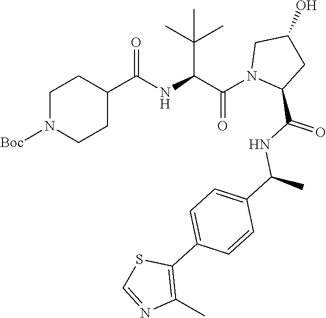 [SMILES: Cc1ncsc1-c1ccc([C@H](C)NC(=O)[C@@H]2C[C@@H](O)CN2C(=O)[C@@H](NC(=O)C2CCN(C(=O)OC(C)(C)C)CC2)C(C)(C)C)cc1]

To a mixture of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (200 mg, 0.45 mmol, 1 eq), 1-tert-butoxycarbonylpiperidine-4-carboxylic acid (113 mg, 0.49 mmol, 1.1 eq), 1-hydroxybenzotriazole (91 mg, 0.67 mmol, 1.5 eq), and diidopropyethylamine (174 mg, 1.35 mmol, 3 eq) in DMF (10 mL) was added N-(3-dimethylaminopropyl)-N-ethylcarbodiimide hydrochloride (129 mg, 0.67 mmol, 1.5 eq), and the reaction mixture was stirred at 25° C. for 16 hours. Water (20 mL) was added, and the resulting mixture was extracted with EtOAc (3×30 mL). The combined organic extracts were washed with brine (3×30 mL), dried over anhydrous Na₂SO₄, filtered, and concentrated. The resulting residue was purified by prep-TLC (CH₂Cl₂/CH₃OH=10/1) to afford tert-butyl 4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]piperidine-1-carboxylate (100 mg, 0.15 mmol, 34% yield) as a white solid. LC/MS (ESI) m/z: 656.4 [M+H]⁺.

### Step 2: Preparation of N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

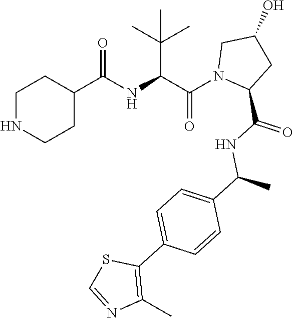 [SMILES: Cc1ncsc1-c1ccc([C@H](C)NC(=O)[C@@H]2C[C@@H](O)CN2C(=O)[C@@H](NC(=O)C2CCNCC2)C(C)(C)C)cc1]

To a mixture of tert-butyl 4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]piperidine-1-carboxylate (50 mg, 0.08 mmol, 1 eq) in CH₂Cl₂ (5 mL) was added trifluoroacetic acid (154 mg, 1.35 mmol, 17.7 eq), and the reaction mixture was stirred at 25° C. for 1 hour. The mixture was concentrated to afford N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide (50 mg, 0.07 mmol, 98% yield, trifluoroacetic acid salt) as a yellow oil.

### Step 3: Preparation of 1-[(2R)-2-[6-chloro-8-fluoro-7-(3-hydroxy-1-naphthyl)-4-(4-prop-2-enoylpiperazin-1-yl)quinazolin-2-yl]oxypropyl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

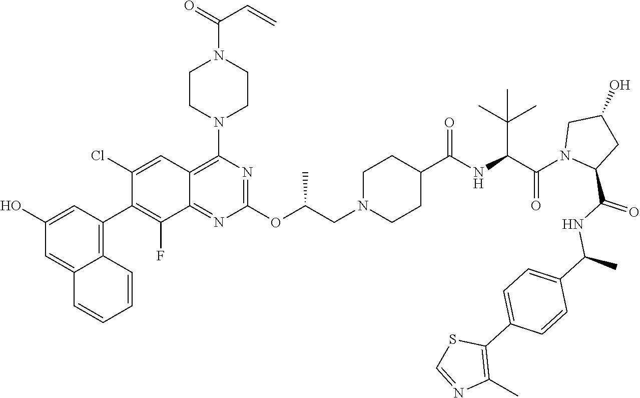 [SMILES: C=CC(=O)N1CCN(c2nc(O[C@H](C)CN3CCC(C(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

To N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide (50 mg, 0.07 mmol, 1 eq, TFA salt) in CH₃OH (5 mL) and CH₂Cl₂ (5 mL) was added NaOAc (61 mg, 0.75 mmol, 10 eq), and the resulting mixture was stirred at 25° C. for 0.2 hour. (2R)-2-[6-Chloro-8-fluoro-7-(3-hydroxy-1-naphthyl)-4-(4-prop-2-enoylpiperazin-1-yl)quinazolin-2-yl]oxypropanal (44 mg, 0.08 mol, 1.1 eq), acetic acid (0.4 g, 0.007 mmol, 0.1 eq), and NaBH₃CN (7 mg, 0.11 mmol, 1.5 eq) were added, and the reaction mixture was stirred at 25° C. for 1 hour. The mixture was concentrated, and the resulting residue was purified by semi-preparative reverse phase HPLC (25%-55% CH₃CN in water (0.225% formic acid)) to afford 1-[(2R)-2-[6-chloro-8-fluoro-7-(3-hydroxy-1-naphthyl)-4-(4-prop-2-enoylpiperazin-1-yl)quinazolin-2-yl]oxypropyl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide (9.3 mg, 0.008 mmol, 11% yield, 97% purity) as a white solid. LC/MS (ESI) m/z: 1074.5 [M+H]⁺. ¹H-NMR (400 MHz, DMSO-d₆) δ 10.02 (d, J=5.2 Hz, 1H), 8.99 (s, 1H), 8.37 (d, J=7.6 Hz, 1H), 8.01 (s, 1H), 7.88-7.76 (m, 1H), 7.66 (d, J=9.6 Hz, 1H), 7.48-7.35 (m, 5H), 7.29 (d, J=2.0 Hz, 1H), 7.25-7.18 (m, 2H), 7.08 (d, J=2.4 Hz, 1H), 6.86-6.82 (m, 1H), 6.21-6.17 (m, 1H), 5.79-5.69 (m, 1H), 5.44-5.40 (m, 1H), 5.09 (d, J=2.4 Hz, 1H), 4.91 (t, J=7.2 Hz, 1H), 4.55-4.37 (m, 2H), 4.27 (d, J=1.2 Hz, 1H), 3.97-3.76 (m, 8H), 3.64-3.51 (m, 2H), 3.09-2.84 (m, 3H), 2.66-2.57 (m, 1H), 2.46 (s, 3H), 2.08-1.73 (m, 5H), 1.66-1.59 (m, 1H), 1.50-1.30 (m, 9H), 0.91 (s, 9H).

The following compounds can be prepared in an analogous manner to 1-[(2R)-2-[6-chloro-8-fluoro-7-(3-hydroxy-1-naphthyl)-4-(4-prop-2-enoylpiperazin-1-yl)quinazolin-2-yl]oxypropyl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide

### 1. (2S,4R)-1-[(2S)-2-[3-[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]propanoylamino]-3,3-dimethyl-butanoyl]-4-hydroxy-N-[(1S)-1-[4-(4-methylthiazol-5-yl)phenyl]ethyl]pyrrolidine-2-carboxamide

### Step 3: Preparation of (2S,4R)-1-[(2S)-2-[3-[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]propanoylamino]-3,3-dimethyl-butanoyl]-4-hydroxy-N-[(1S)-1-[4-(4-methylthiazol-5-yl)phenyl]ethyl]pyrrolidine-2-carboxamide

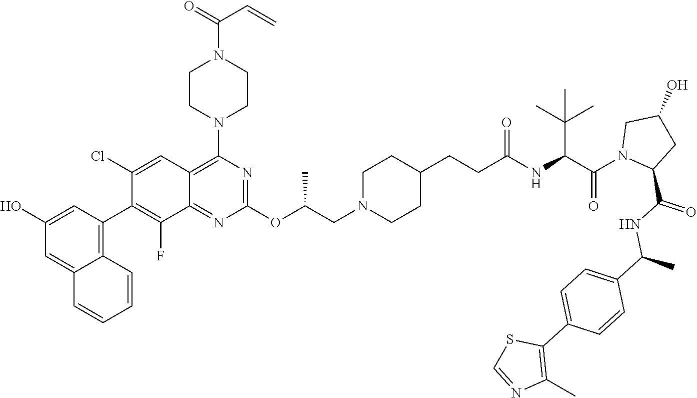 [SMILES: C=CC(=O)N1CCN(c2nc(O[C@H](C)CN3CCC(CCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(formic acid salt, white solid). LC/MS (ESI) m/z: 1102.5 [M+H]⁺. ¹H-NMR (400 MHz, CD₃OD) δ 8.95-8.75 (m, 1H), 8.53 (s, 1H), 8.03 (d, J=2.4 Hz, 1H), 7.75 (d, J=8.4 Hz, 1H), 7.48-7.29 (m, 5H), 7.29-7.16 (m, 3H), 7.04 (t, J=2.4 Hz, 1H), 6.81 (m, 1H), 6.29 (d, J=16.8 Hz, 1H), 5.85-5.78 (d, J=12.0 Hz, 1H), 5.64 (d, J=4.8 Hz, 1H), 5.03-4.96 (m, 1H), 4.59-4.31 (m, 3H), 4.02 (s, 4H), 3.96-3.82 (m, 5H), 3.73 (dd, J=3.6, 10.8 Hz, 1H), 3.65-3.49 (m, 1H), 3.35 (m, 1H), 3.25 (d, J=12.8 Hz, 1H), 3.15-3.05 (m, 1H), 2.95-2.83 (m, 1H), 2.68-2.39 (m, 5H), 2.35-2.11 (m, 3H), 1.94 (m, 1H), 1.77 (m, 2H), 1.57-1.46 (m, 5H), 1.43 (d, J=6.0 Hz, 3H), 1.35-1.23 (m, 2H), 1.06-0.96 (s, 9H).

### 2. (2S,4R)-1-[(2S)-2-[[2-[1-[(2R)-2-[6-chloro-8-fluoro-7-(3-hydroxy-1-naphthyl)-4- (4-prop-2-enoylpiperazin-1-yl)quinazolin-2-yl]oxypropyl]-4-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

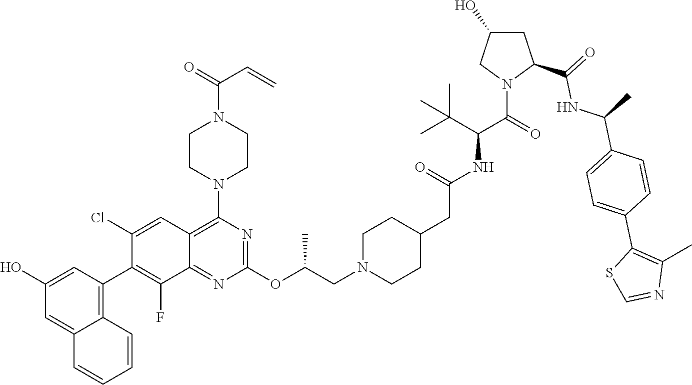 [SMILES: C=CC(=O)N1CCN(c2nc(O[C@H](C)CN3CCC(CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

(white solid). LC/MS (ESI) m/z: 1088.4 [M+H]⁺. ¹H-NMR (400 MHz, DMSO-d₆) δ 10.17-9.93 (m, 1H), 8.99 (s, 1H), 8.37 (br d, J=7.7 Hz, 1H), 8.02 (s, 1H), 7.85-7.73 (m, 2H), 7.48-7.41 (m, 3H), 7.40-7.36 (m, 2H), 7.30 (d, J=2.3 Hz, 1H), 7.22 (br d, J=3.9 Hz, 2H), 7.08 (dd, J=2.4, 4.8 Hz, 1H), 6.84 (dd, J=10.4, 16.6 Hz, 1H), 6.19 (dd, J=2.3, 16.7 Hz, 1H), 5.79-5.73 (m, 1H), 5.46-5.37 (m, 1H), 5.14-5.04 (m, 1H), 4.99-4.86 (m, 1H), 4.52-4.37 (m, 2H), 4.27 (br s, 1H), 3.93 (br s, 4H), 3.88-3.76 (m, 4H), 3.59 (br s, 2H), 3.33 (br s, 4H), 3.00-2.85 (m, 2H), 2.46 (s, 3H), 2.19-2.11 (m, 1H), 2.01 (br d, J=7.2 Hz, 3H), 1.78 (ddd, J=4.6, 8.3, 12.8 Hz, 1H), 1.65-1.48 (m, 3H), 1.37 (br d, J=6.8 Hz, 2H), 1.31 (br d, J=6.0 Hz, 3H), 1.17-1.02 (m, 2H), 0.91 (br d, J=7.9 Hz, 9H).

### Exemplary Synthesis of (2S,4R)—N-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide and (2S,4R)—N-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

### Step 1: Preparation of 2-benzyloxyethyl 4-methylbenzenesulfonate

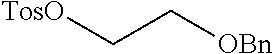

To a solution of 2-benzyloxyethanol (50 g, 328.54 mmol, 46.73 mL, 1 eq) and KOH (22.12 g, 394.24 mmol, 1.2 eq) in THF (200 mL) was added toluenesulfonyl chloride (56.37 g, 295.68 mmol, 0.9 eq), and the reaction mixture was stirred at 25° C. for 1 hour. EtOAc (1 L) was added, the resulting mixture was filtered, and the filtrate was washed by brine (200 mL), dried over anhydrous Na₂SO₄, filtered, and concentrated. The resulting residue was purified by flash chromatography on SiO₂ (15-25% EtOAc in petroleum ether) to afford 2-benzyloxyethyl 4-methylbenzenesulfonate (75 g, 243.82 mmol, 74% yield, 99% purity) as a yellow oil. ¹H-NMR (400 MHz, CDCl₃) δ 7.85-7.79 (m, 2H), 7.38-7.31 (m, 4H), 7.30-7.26 (m, 2H), 7.31 (s, 1H), 4.51 (s, 2H), 4.26-4.19 (m, 2H), 3.73-3.65 (m, 2H), 2.50-2.40 (m, 3H).

### Step 2: Preparation of tert-butyl4-(2-benzyloxyethoxy)piperidine-1-carboxylate

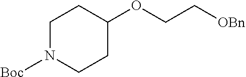 [SMILES: CC(C)(C)OC(=O)N1CCC(OCCOCc2ccccc2)CC1]

To a solution of tert-butyl 4-hydroxypiperidine-1-carboxylate (10.35 g, 51.41 mmol, 1.05 eq) in DMF (150 mL) at 0° C. was added NaH (2.15 g, 53.86 mmol, 60% purity, 1.1 eq), and the resulting mixture was stirred for 0.5 hours at 0° C. . 2-Benzyloxyethyl 4-methylbenzenesulfonate (15 g, 48.96 mmol, 1 eq) was then added, and the reaction mixture was stirred at 25° C. for 1 hour. NH₄Cl solution (200 mL) was added, and the resulting mixture was extracted with EtOAc (3×200 mL). The combined organic extracts were washed with brine (2×100 mL), dried over anhydrous Na₂SO₄, filtered, and concentrated. The resulting residue was purified by flash chromatography on SiO₂ (10-20% EtOAc in petroleum ether) to afford tert-butyl 4-(2-benzyloxyethoxy)piperidine-1-carboxylate (14.5) as a colorless oil. LC/MS (ESI) m/z: 236.1 [M-Boc+1]⁺. ¹H-NMR (400 MHz, CDCl₃) δ 7.41-7.30 (m, 5H), 4.60 (s, 2H), 3.80 (br d, J=9.9 Hz, 2H), 3.71-3.62 (m, 4H), 3.55-3.46 (m, 1H), 3.08 (ddd, J=3.5, 9.5, 13.3 Hz, 2H), 1.86 (br d, J=7.9 Hz, 2H), 1.60-1.50 (m, 2H), 1.47 (s, 9H).

### Step 3: Preparation of tert-butyl 4-(2-benzyloxyethoxy)piperidine-1-carboxylate

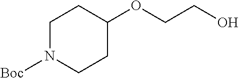 [SMILES: CC(C)(C)OC(=O)N1CCC(OCCO)CC1]

To a solution of tert-butyl 4-(2-benzyloxyethoxy)piperidine-1-carboxylate (5 g, 14.91 mmol, 1 eq) in CH₃OH (40 mL) was added Pd/C (0.5 g, 10% purity) under nitrogen, and the resulting suspension was degassed under vacuum and purged with H₂ several times. The reaction mixture was stirred under H₂ (15 psi) at 25° C. for 12 hours, and then filtered and concentrated to afford tert-butyl 4-(2-hydroxyethoxy)piperidine-1-carboxylate (3 g, 12.23 mmol, 82% yield) as a colorless oil. ¹H-NMR (400 MHz, CDCl₃) δ 3.86-3.72 (m, 4H), 3.66-3.58 (m, 2H), 3.56-3.45 (m, 1H), 3.16-3.07 (m, 2H), 2.15-2.05 (m, 1H), 1.96-1.78 (m, 2H), 1.59-1.49 (m, 2H), 1.47 (s, 9H).

### Step 4: Preparation of tert-butyl 4-[2-(p-tolylsulfonyloxy)ethoxy]piperidine-1-carboxylate

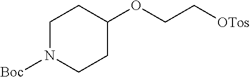

To a mixture of tert-butyl 4-(2-hydroxyethoxy)piperidine-1-carboxylate (7.4 g, 30.17 mmol, 1 eq) and triethylamine (9.16 g, 90.50 mmol, 12.60 mL, 3 eq) in CH₂Cl₂ (70 mL) at 0° C. was added toluenesulfonyl chloride (8.63 g, 45.25 mmol, 1.5 eq), and the reaction mixture was stirred at 25° C. for 12 hours. The mixture was concentrated, and the resulting residue was purified by flash chromatography on SiO₂ (20-50% EtOAc in petroleum ether) to afford tert-butyl 4-[2-(p-tolylsulfonyloxy)ethoxy]piperidine-1-carboxylate (8.7 g, 21.78 mmol, 72% yield) as a yellow oil. ¹H-NMR (400 MHz, CDCl₃) δ 7.80 (d, J=8.3 Hz, 2H), 7.34 (d, J=8.1 Hz, 2H), 4.18-4.13 (m, 2H), 3.76-3.70 (m, 2H), 3.67-3.60 (m, 4H), 3.47-3.39 (m, 1H), 3.14-3.01 (m, 2H), 2.46-2.43 (m, 3H), 1.90-1.78 (m, 2H), 1.45 (s, 9H).

### Step 5: Preparation of tert-butyl 4-[2-[2-[[[(2S,4R)-4-hydroxy-1-[3-methyl-2-(3-methylisoxazol-5-yl)butanoyl]pyrrolidine-2-carbonyl]amino]methyl]-5-(4-methylthiazol-5-yl)phenoxy]ethoxy]piperidine-1-carboxylate

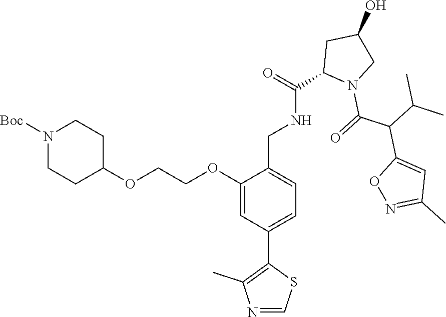 [SMILES: Cc1cc(C(C(=O)N2C[C@H](O)C[C@H]2C(=O)NCc2ccc(-c3scnc3C)cc2OCCOC2CCN(C(=O)OC(C)(C)C)CC2)C(C)C)on1]

To a mixture of tert-butyl 4-[2-(p-tolylsulfonyloxy)ethoxy]piperidine-1-carboxylate (280 mg, 0.70 mmol, 1 eq) and (2S,4R)-4-hydroxy-N-[[2-hydroxy-4-(4-methylthiazol-5-yl)phenyl]methyl]-1-[3-methyl-2-(3-methylisoxazol-5-yl)butanoyl]pyrrolidine-2-carboxamide (350 mg, 0.70 mmol, 1 eq) in CH₃CN (8 mL) was added K₂CO₃ (194 mg, 1.40 mmol, 2 eq), and the reaction mixture was stirred at 85° C. for 16 hours. The mixture was filtrated, and the filtrate was purified by prep-TLC (CH₂Cl₂:CH₃OH=10:1) to give tert-butyl 4-[2-[2-[[[(2S,4R)-4-hydroxy-1-[3-methyl-2-(3-methylisoxazol-5-yl)butanoyl]pyrrolidine-2-carbonyl]amino]methyl]-5-(4-methylthiazol-5-yl)phenoxy]ethoxy]piperidine-1-carboxylate (400 mg, 0.55 mmol, 79% yield) as a yellow solid. LC/MS (ESI) m/z: 726.3 [M+H]⁺. This material was purified further by prep-HPLC (55% isopropanol in water (0.1% NH₄OH)) to give tert-butyl 4-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]piperidine-1-carboxylate (200 mg, 0.27 mmol, 48% yield, 97% purity) as a white solid and tert-butyl 4-[2-[2-[[[(2S,4R)-4-hydroxy-1-[(2S)-3-methyl-2-(3-methylisoxazol-5-yl)butanoyl]pyrrolidine-2-carbonyl]amino]methyl]-5-(4-methylthiazol-5-yl)phenoxy]ethoxy]piperidine-1-carboxylate (150 mg, 0.20 mmol, 36% yield, 97% purity) as a white solid.

### Step 6: Preparation of (2S,4R)-4-hydroxy-1-[(2R)-3-methyl-2-(3-methylisoxazol-5-yl)butanoyl]-N-[[4-(4-methylthiazol-5-yl)-2-[2-(4-piperidyloxy)ethoxy]phenyl]methyl]pyrrolidine-2-carboxamide

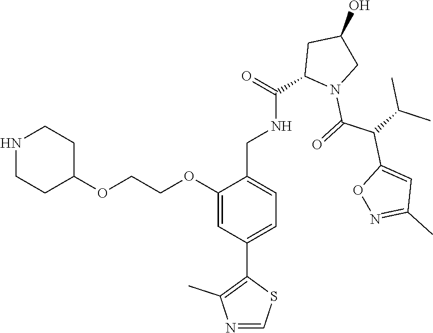

To a solution of tert-butyl 4-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]piperidine-1-carboxylate (200 mg, 0.27 mmol, 1 eq) in CH₂Cl₂ (5 mL) was added HCl (4N in dioxane, 1 mL), and the reaction mixture was stirred at 25° C. for 1 hour. The mixture was concentrated, and the resulting residue was purified by prep-HPLC (15-36% CH₃CN in water (0.1% TFA)) to give (2S,4R)-4-hydroxy-1-[(2R)-3-methyl-2-(3-methylisoxazol-5-yl)butanoyl]-N-[[4-(4-methylthiazol-5-yl)-2-[2-(4-piperidyloxy)ethoxy]phenyl]methyl]pyrrolidine-2-carboxamide (80 mg, 0.12 mmol, 43.4% yield, 99% purity, HCl salt) as a yellow solid. LC/MS (ESI) m/z: 626.4 [M+H]⁺.

### Step 7: Preparation of tert-butyl 4-[6-chloro-8-fluoro-2-[(1R)-2-[4-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]-1-piperidyl]-1-methyl-ethoxy]-7-(3-hydroxy-1-naphthyl)quinazolin-4-yl]piperazine-1-carboxylate

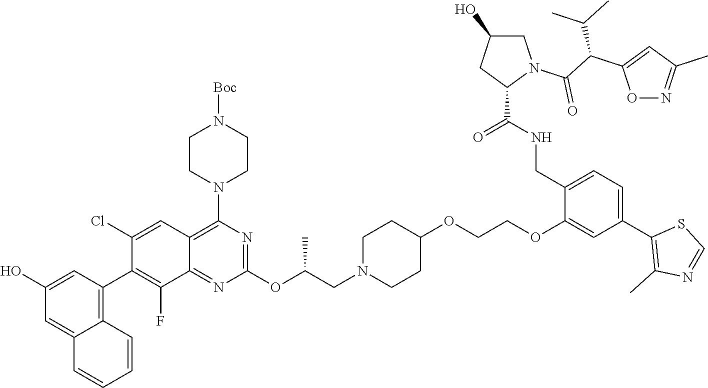 [SMILES: Cc1cc([C@H](C(=O)N2C[C@H](O)C[C@H]2C(=O)NCc2ccc(-c3scnc3C)cc2OCCOC2CCN(C[C@@H](C)Oc3nc(N4CCN(C(=O)OC(C)(C)C)CC4)c4cc(Cl)c(-c5cc(O)cc6ccccc56)c(F)c4n3)CC2)C(C)C)on1]

To a solution of (2S,4R)-4-hydroxy-1-[(2R)-3-methyl-2-(3-methylisoxazol-5-yl)butanoyl]-N-[[4-(4-methylthiazol-5-yl)-2-[2-(4-piperidyloxy)ethoxy]phenyl]methyl]pyrrolidine-2-carboxamide (80 mg, 0.12 mmol, 1 eq, HCl salt) in CH₂Cl₂ (1 mL) and CH₃OH (3 mL) was added NaOAc (20 mg, 0.24 mmol, 2 eq), and the resulting mixture was stirred at 25° C. for 30 minutes. tert-Butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (70 mg, 0.12 mmol, 1 eq) and acetic acid (14 mg, 0.24 mmol, 2 eq) were then added, and the resulting mixture was stirred for 15 minutes and cooled to 0° C. NaBH₃CN (23 mg, 0.36 mmol, 3 eq) was then added, and the reaction mixture was stirred at 25° C. for 16 hours. The mixture was concentrated, and the resulting residue was purified by prep-TLC (CH₂Cl₂:CH₃OH=8:1) to give tert-butyl 4-[6-chloro-8-fluoro-2-[(1R)-2-[4-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]-1-piperidyl]-1-methyl-ethoxy]-7-(3-hydroxy-1-naphthyl)quinazolin-4-yl]piperazine-1-carboxylate (80 mg, 0.07 mmol, 56% yield) as a yellow solid. LC/MS (ESI) m/z: 1189.3 [M+H]⁺.

### Step 8: Preparation of (2S,4R)—N-[[2-[2-[[1-[(2R)-2-[6-chloro-8-fluoro-7-(3-hydroxy-1-naphthyl)-4-piperazin-1-yl-quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

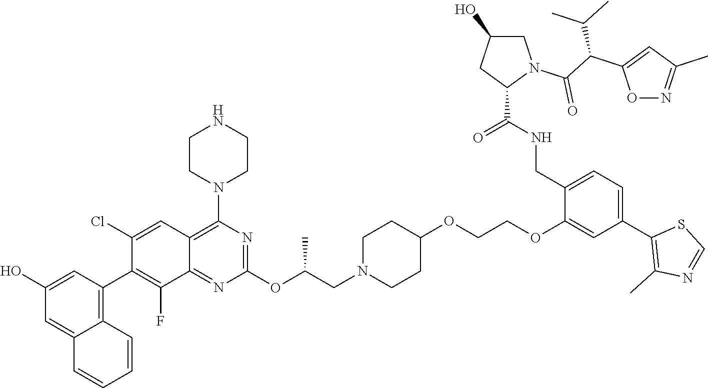 [SMILES: Cc1cc([C@H](C(=O)N2C[C@H](O)C[C@H]2C(=O)NCc2ccc(-c3scnc3C)cc2OCCOC2CCN(C[C@@H](C)Oc3nc(N4CCNCC4)c4cc(Cl)c(-c5cc(O)cc6ccccc56)c(F)c4n3)CC2)C(C)C)on1]

To a solution of tert-butyl 4-[6-chloro-8-fluoro-2-[(1R)-2-[4-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]-1-piperidyl]-1-methyl-ethoxy]-7-(3-hydroxy-1-naphthyl)quinazolin-4-yl]piperazine-1-carboxylate (80 mg, 0.07 mmol, 1 eq) in CH₂Cl₂ (10 mL) was added trifluoroacetic acid (1.54 g, 13.51 mmol, 1 mL, 201.04 eq), and the reaction mixture was stirred at 25° C. for 1 hour. The mixture was concentrated to give (2S,4R)—N-[[2-[2-[[1-[(2R)-2-[6-chloro-8-fluoro-7-(3-hydroxy-1-naphthyl)-4-piperazin-1-yl-quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide (80 mg, 0.07 mmol, 99% yield, TFA salt) as a yellow oil. LC/MS (ESI) m/z: 545.9 [M/2+H]⁺.

### Step 9: Preparation of (2S,4R)—N-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

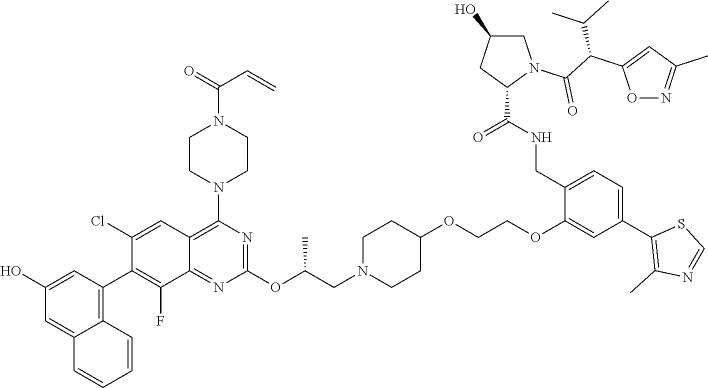 [SMILES: C=CC(=O)N1CCN(c2nc(O[C@H](C)CN3CCC(OCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1]

To a solution of (2S,4R)—N-[[2-[2-[[1-[(2R)-2-[6-chloro-8-fluoro-7-(3-hydroxy-1-naphthyl)-4-piperazin-1-yl-quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide (80 mg, 0.07 mmol, 1 eq, TFA salt) in DMF (1 mL) and CH₂Cl₂ (5 mL) was added 2,6-lutidine (36 mg, 0.33 mmol, 0.04 mL, 5 eq), and the resulting mixture was stirred at −70° C. for 5 minutes. Prop-2-enoyl chloride (6 mg, 0.07 mmol, 1 eq) was then added, and the reaction mixture was stirred at −70° C. for 15 minutes. Water (5 mL) was added, and the resulting mixture was extracted with CH₂Cl₂ (60 mL). The organic extract was dried over anhydrous Na₂SO₄, filtered, and concentrated. The resulting residue was purified by prep-HPLC (30-60% CH₃CN in water (0.225% formic acid)) to give (2S,4R)—N-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide (27.4 mg, 0.02 mmol, 35.3% yield, 98% purity, formic acid salt) as a white solid. LC/MS (ESI) m/z: 1144.5 [M+H]⁺. ¹H-NMR (400 MHz, DMSO-d₆) δ 10.01 (br s, 1H), 8.97 (s, 1H), 8.80-8.21 (m, 1H), 8.01 (s, 1H), 7.81 (dd, J=5.4, 8.4 Hz, 1H), 7.49-7.36 (m, 1H), 7.33-7.16 (m, 4H), 7.10-7.06 (m, 1H), 7.04-7.01 (m, 1H), 7.00-6.93 (m, 1H), 6.84 (dd, J=10.4, 16.8 Hz, 1H), 6.22-6.15 (m, 2H), 5.78-5.73 (m, 1H), 5.44-5.34 (m, 1H), 5.19-4.89 (m, 1H), 4.51-4.44 (m, 1H), 4.38-4.31 (m, 1H), 4.30-4.12 (m, 4H), 3.93 (br s, 4H), 3.89-3.68 (m, 8H), 3.62-3.49 (m, 2H), 2.87-2.59 (m, 3H), 2.48-2.43 (m, 3H), 2.42-2.32 (m, 1H), 2.27 (br dd, J=6.4, 15.2 Hz, 1H), 2.21-2.16 (m, 2H), 2.13 (d, J=2.0 Hz, 3H), 2.10-2.01 (m, 1H), 1.95-1.86 (m, 1H), 1.77 (br s, 2H), 1.42-1.28 (m, 5H), 0.97 (d, J=6.8 Hz, 2H), 0.80 (d, J=6.8 Hz, 2H), 0.69 (d, J=6.4 Hz, 1H), 0.56 (dd, J=2.0, 6.8 Hz, 1H).

### (2S,4R)—N-[[2-[2-[2-[2-[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

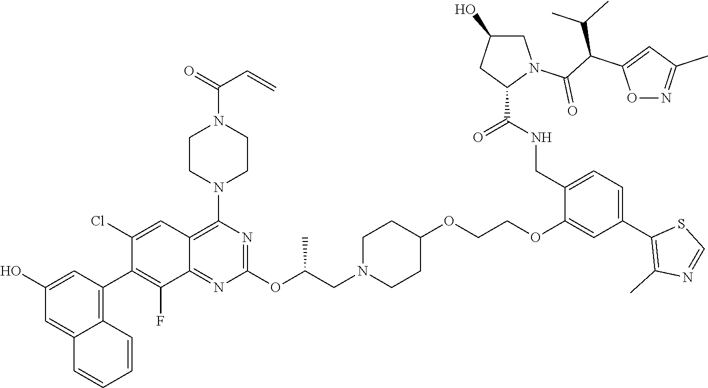 [SMILES: C=CC(=O)N1CCN(c2nc(O[C@H](C)CN3CCC(OCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

(formic acid salt, white solid). LC/MS (ESI) m/z: 1144.4 [M+H]⁺. ¹H-NMR (400 MHz, DMSO-d₆) δ 10.04 (br s, 1H), 8.97 (s, 1H), 8.65-8.27 (m, 1H), 8.01 (s, 1H), 7.81 (dd, J=5.2, 8.4 Hz, 1H), 7.48-7.38 (m, 1H), 7.35 (d, J=7.6 Hz, 1H), 7.29 (t, J=2.0 Hz, 1H), 7.25-7.17 (m, 2H), 7.10-6.97 (m, 3H), 6.84 (dd, J=10.4, 16.8 Hz, 1H), 6.26-6.14 (m, 2H), 5.94-5.72 (m, 1H), 5.39 (dt, J=6.0, 12.4 Hz, 1H), 5.25-4.61 (m, 1H), 4.42-4.14 (m, 6H), 3.96-3.71 (m, 12H), 3.62-3.49 (m, 2H), 2.78 (br d, J=15.6 Hz, 2H), 2.68-2.58 (m, 1H), 2.48-2.43 (m, 3H), 2.42-2.34 (m, 1H), 2.30-2.24 (m, 1H), 2.21 (s, 4H), 2.14-2.09 (m, 1H), 2.08-1.98 (m, 1H), 1.92 (ddd, J=4.8, 7.6, 12.8 Hz, 1H), 1.77 (br s, 2H), 1.45-1.28 (m, 5H), 1.00-0.88 (m, 3H), 0.76 (d, J=6.4 Hz, 3H).

The following compounds were prepared following a similar synthetic sequence to (2S,4R)—N-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide and (2S,4R)—N-[[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

### 1. (2S,4R)—N-[[2-[2-[2-[[1-[(2R)-2-[6-chloro-8-fluoro-7-(3-hydroxy-1-naphthyl)-4- (4-prop-2-enoylpiperazin-1-yl)quinazolin-2-yl]oxypropyl]-4-piperidyl]oxy]ethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

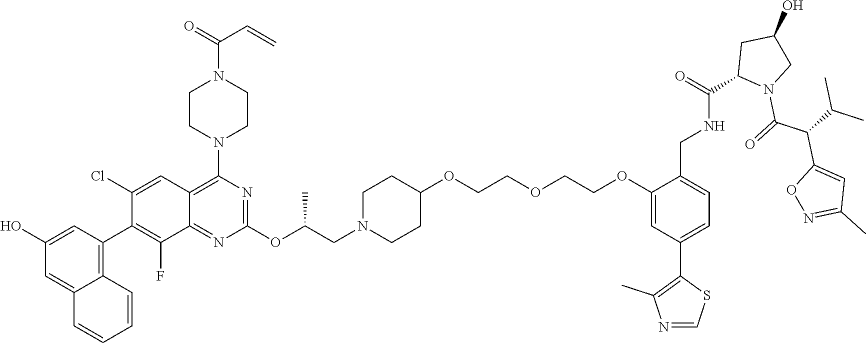 [SMILES: C=CC(=O)N1CCN(c2nc(O[C@H](C)CN3CCC(OCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1]

(formic acid salt, white solid). LC/MS (ESI) m/z: 1189.5 [M+H]⁺. ¹H-NMR (400 MHz, DMSO-d₆) δ 10.00 (s, 1H), 8.96 (s, 1H), 8.27-8.16 (m, 1H), 8.05-7.97 (m, 1H), 7.85-7.72 (m, 1H), 7.49-7.39 (m, 1H), 7.32-7.16 (m, 4H), 7.10-7.04 (m, 1H), 7.03-6.99 (m, 1H), 6.96 (d, J=7.5 Hz, 1H), 6.83 (dd, J=10.4, 16.6 Hz, 1H), 6.24-6.13 (m, 2H), 5.78-5.70 (m, 1H), 5.45-5.29 (m, 1H), 5.11 (s, 1H), 4.48-4.14 (m, 6H), 3.93-3.75 (m, 9H), 3.59-3.43 (m, 5H), 3.23 (d, J=11.0 Hz, 3H), 2.75 (s, 2H), 2.60 (dd, J=6.5, 12.7 Hz, 1H), 2.46-2.43 (m, 3H), 2.39-2.34 (m, 1H), 2.30-2.17 (m, 2H), 2.14-2.01 (m, 5H), 1.94-1.85 (m, 1H), 1.70 (s, 2H), 1.37-1.21 (m, 5H), 0.96 (d, J=6.7 Hz, 2H), 0.79 (d, J=6.7 Hz, 2H), 0.67 (d, J=6.5 Hz, 1H), 0.55 (d, J=6.5 Hz, 1H).

### 2. (2S,4R)—N-[[2-[2-[2-[[1-[(2R)-2-[6-chloro-8-fluoro-7-(3-hydroxy-1-naphthyl)-4- (4-prop-2-enoylpiperazin-1-yl)quinazolin-2-yl]oxypropyl]-4-piperidyl]oxy]ethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

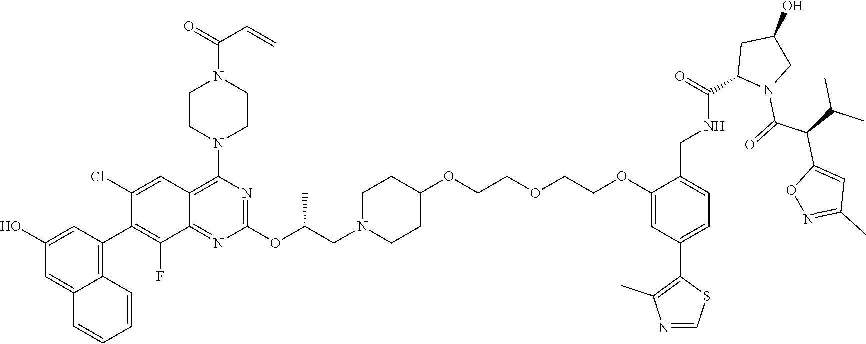 [SMILES: C=CC(=O)N1CCN(c2nc(O[C@H](C)CN3CCC(OCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

(formic acid salt, white solid). LC/MS (ESI) m/z: 1188.6 [M+H]⁺. ¹H-NMR (400 MHz, DMSO-d₆) δ 10.00 (s, 1H), 8.96 (s, 1H), 8.34-8.24 (m, 1H), 8.00 (s, 1H), 7.80 (dd, J=4.3, 8.0 Hz, 1H), 7.43 (d, J=4.4 Hz, 1H), 7.34 (d, J=7.6 Hz, 1H), 7.28 (s, 1H), 7.24-7.18 (m, 2H), 7.08-6.96 (m, 3H), 6.83 (dd, J=10.5, 16.6 Hz, 1H), 6.22-6.12 (m, 2H), 5.74 (d, J=12.5 Hz, 1H), 5.43-5.29 (m, 1H), 5.08 (s, 1H), 4.39-4.14 (m, 6H), 3.92-3.72 (m, 10H), 3.59-3.42 (m, 4H), 3.26-3.21 (m, 3H), 2.85-2.70 (m, 2H), 2.59 (s, 1H), 2.45-2.42 (m, 3H), 2.37 (s, 1H), 2.26 (dd, J=6.5, 16.0 Hz, 1H), 2.19 (s, 2H), 2.13-1.82 (m, 5H), 1.70 (s, 2H), 1.30 (d, J=6.1 Hz, 5H), 0.95-0.89 (m, 3H), 0.75 (d, J=6.7 Hz, 3H).

### 3. (2S,4R)—N-[[2-[2-[2-[2-[[1-[(2R)-2-[6-chloro-8-fluoro-7-(3-hydroxy-1- naphthyl)-4-(4-prop-2-enoylpiperazin-1-yl)quinazolin-2-yl]oxypropyl]-4-piperidyl]oxy]ethoxy]ethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

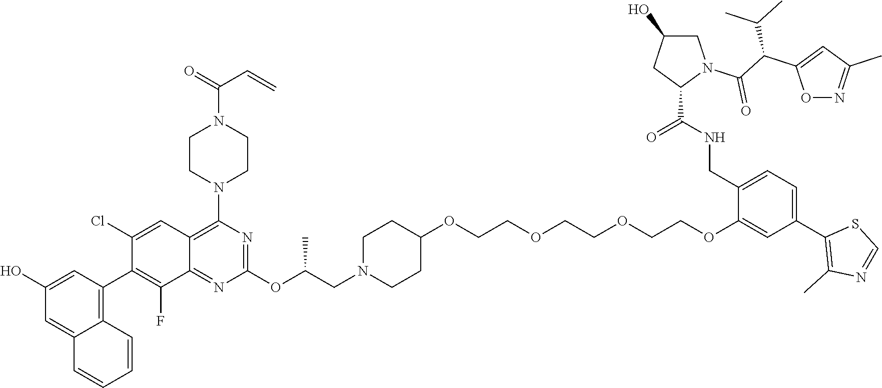 [SMILES: C=CC(=O)N1CCN(c2nc(O[C@H](C)CN3CCC(OCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1]

(formic acid salt, white solid). LC/MS (ESI) m/z: 1233.4 [M+H]⁺. ¹H-NMR (400 MHz, DMSO-d₆) δ 10.11-9.91 (m, 1H), 8.97 (s, 1H), 8.27-8.20 (m, 1H), 8.01 (s, 1H), 7.83-7.79 (m, 1H), 7.47-7.41 (m, 1H), 7.31-7.20 (m, 4H), 7.12-6.93 (m, 3H), 6.85-6.81 (m, 1H), 6.24-6.14 (m, 2H), 5.79-5.71 (m, 1H), 5.39-5.35 (m, 1H), 5.15-4.90 (m, 1H), 4.51-4.45 (m, 1H), 4.20 (d, J=6.0 Hz, 4H), 3.96-3.73 (m, 12H), 3.44 (d, J=3.2 Hz, 14H), 2.85-2.70 (m, 2H), 2.63-2.56 (m, 1H), 2.46 (s, 2H), 2.39-2.34 (m, 1H), 2.30-2.23 (m, 1H), 2.20 (s, 1H), 2.16-2.08 (m, 4H), 1.97-1.86 (m, 1H), 1.73-1.69 (m, 2H), 1.32-1.28 (m, 4H), 0.97 (d, J=6.8 Hz, 2H), 0.80 (d, J=6.8 Hz, 2H), 0.71-0.66 (m, 1H), 0.59-0.54 (m, 1H).

### 4. (2S,4R)—N-[[2-[2-[2-[2-[[1-[(2R)-2-[6-chloro-8-fluoro-7-(3-hydroxy-1- naphthyl)-4-(4-prop-2-enoylpiperazin-1-yl)quinazolin-2-yl]oxypropyl]-4-piperidyl]oxy]ethoxy]ethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

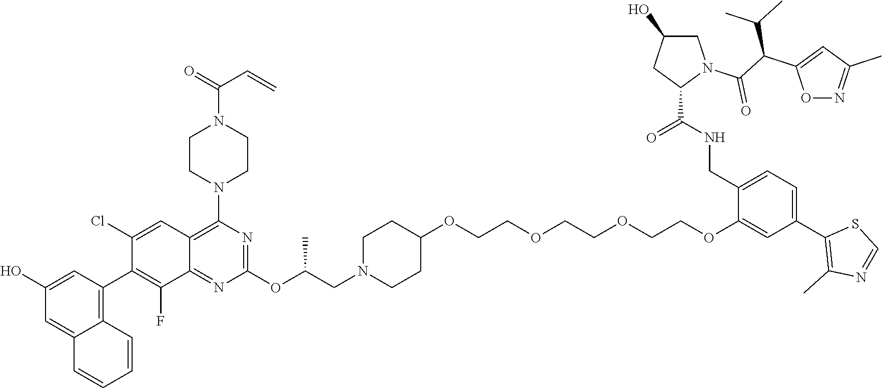 [SMILES: C=CC(=O)N1CCN(c2nc(O[C@H](C)CN3CCC(OCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

(formic acid salt, off-white solid). LC/MS (ESI) m/z: 1233.4 [M+H]⁺. ¹H-NMR (400 MHz, DMSO-d₆) δ 10.25-9.83 (m, 1H), 8.97 (s, 1H), 8.47-8.30 (m, 2H), 8.01 (s, 1H), 7.83-7.79 (m, 1H), 7.47-7.40 (m, 1H), 7.35 (d, J=7.6 Hz, 1H), 7.29 (s, 1H), 7.24-7.19 (m, 2H), 7.08-6.99 (m, 3H), 6.86-6.82 (m, 1H), 6.27-6.12 (m, 2H), 5.82-5.72 (m, 1H), 5.40-5.36 (m, 1H), 5.15-5.07 (m, 1H), 4.44-4.13 (m, 8H), 3.99-3.69 (m, 16H), 3.62-3.52 (m, 7H), 2.30-2.16 (m, 5H), 2.13-2.02 (m, 4H), 1.95-1.87 (m, 1H), 1.75-1.67 (m, 2H), 1.35-1.19 (m, 6H), 0.93 (d, J=6.8 Hz, 3H), 0.76 (d, J=6.8 Hz, 3H).

### 5. (2S,4R)—N-[[2-[2-[2-[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

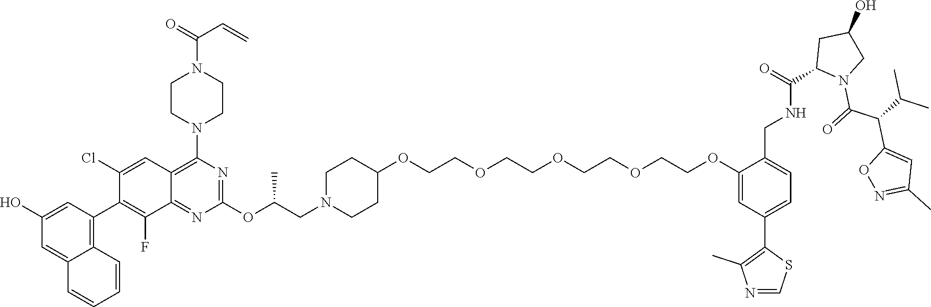 [SMILES: C=CC(=O)N1CCN(c2nc(O[C@H](C)CN3CCC(OCCOCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1]

(TFA salt, white solid). LC/MS (ESI) m/z: 638.8 [M/2+1]⁺. ¹H-NMR (400 MHz, DMSO-d₆) δ 10.06-10.05 (m, 1H), 9.4-9.27 (m, 1H), 9.36-9.20 (m, 1H), 9.36-9.20 (m, 1H), 8.98 (s, 1H), 8.29-8.20 (m, 1H), 8.08 (s, 1H), 7.82-7.80 (m, 1H), 7.45-7.40 (m, 1H), 7.32-7.16 (m, 5H), 7.09-6.97 (m, 3H), 6.83-6.8 (m, 1H), 6.24-6.16 (m, 2H), 5.76-5.72 (m, 1H), 5.63 (s, 1H), 4.51-4.43 (m, 1H), 4.40-4.32 (m, 1H), 4.31-4.18 (m, 3H), 4.16-4.12 (m, 2H), 3.99 (s, 4H), 3.89-3.75 (m, 8H), 3.68-3.57 (m, 10H), 3.20-2.96 (m, 2H), 2.47-2.43 (m, 4H), 2.27-2.21 (m, 1H), 2.20 (s, 1H), 2.14 (s, 3H), 2.08 (s, 3H), 1.96-1.85 (m, 2H), 1.77-1.44 (m, 2H), 1.41-1.34 (m, 3H), 1.27-1.06 (m, 1H), 0.97-0.95 (m, 2H), 0.80-0.78 (m, 2H), 0.70-0.65 (m, 1H), 0.58-0.53 (m, 1H).

### 6. (2S,4R)—N-[[2-[2-[2-[2-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

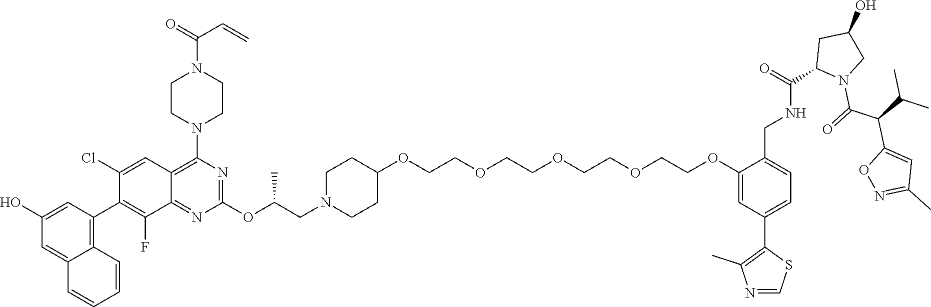 [SMILES: C=CC(=O)N1CCN(c2nc(O[C@H](C)CN3CCC(OCCOCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

(TFA salt, white solid). LC/MS (ESI) m/z: 638.9 [M/2+1]⁺. ¹H-NMR (400 MHz, DMSO-d₆) δ 10.03 (s, 1H), 9.20 (s, 1H), 8.98 (s, 1H), 8.35-8.3 (m, 1H), 8.07 (s, 1H), 7.82-7.60 (m, 1H), 7.45-7.40 (m, 1H), 7.37-7.28 (m, 2H), 7.25-7.19 (m, 2H), 7.09-6.98 (m, 3H), 6.83-6.80 (m, 1H), 6.24-6.16 (m, 2H), 5.79-5.74 (m, 1H), 5.63 (s, 1H), 4.42-4.33 (m, 2H), 4.27-4.23 (m, 2H), 4.17-4.15 (m, 2H), 3.99-3.95 (m, 4H), 3.87-3.85 (m, 3H), 3.77-3.74 (m, 6H), 3.64-3.59 (m, 3H), 3.55-3.54 (m, 8H), 3.20-2.96 (m, 2H), 2.46 (s, 3H), 2.44 (s, 1H), 2.27-2.22 (m, 2H), 2.20 (s, 3H), 2.14-1.98 (m, 4H), 1.96-1.86 (m, 3H), 1.76-1.56 (m, 2H), 1.46 (s, 1H), 1.37-1.34 (m, 3H), 0.98-0.88 (m, 3H), 0.81-0.72 (m, 3H).

### 7. (2S,4R)—N-[[2-[2-[2-[2-[2-[2-[[1-[(2R)-2-[6-chloro-8-fluoro-7-(3- hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

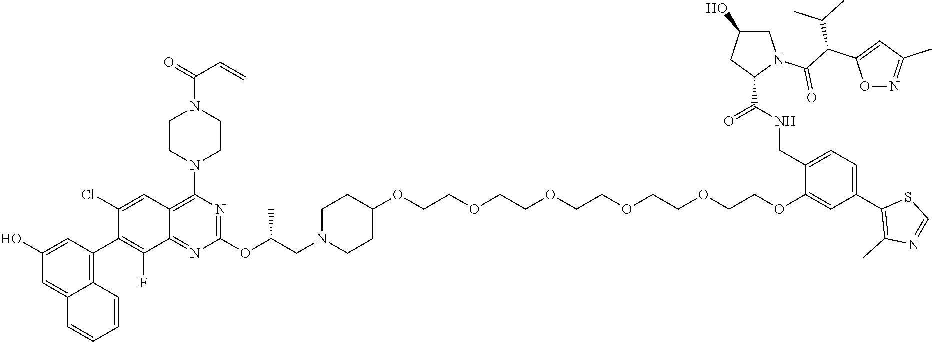 [SMILES: C=CC(=O)N1CCN(c2nc(O[C@H](C)CN3CCC(OCCOCCOCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1]

(formic acid, white solid). LC/MS (ESI) m/z: 661.8 [M/2+H]⁺. ¹H-NMR (400 MHz, DMSO-d₆) δ 10.32-9.78 (m, 1H), 8.97 (s, 1H), 8.27 (s, 1H), 8.00 (s, 1H), 7.80 (d, J=8.1 Hz, 1H), 7.43 (s, 1H), 7.34-7.15 (m, 4H), 7.06 (dd, J=2.4, 4.9 Hz, 1H), 7.02 (d, J=1.5 Hz, 1H), 6.99-6.95 (m, 1H), 6.83 (dd, J=10.4, 16.8 Hz, 1H), 6.25-6.12 (m, 2H), 5.79-5.69 (m, 1H), 5.45-5.30 (m, 1H), 5.19-4.87 (m, 1H), 4.51-4.13 (m, 6H), 3.94-3.89 (m, 3H), 3.87-3.72 (m, 7H), 3.62-3.56 (m, 3H), 3.55-3.50 (m, 3H), 3.49-3.42 (m, 13H), 2.85-2.69 (m, 2H), 2.64-2.58 (m, 1H), 2.46-2.44 (m, 3H), 2.40-2.35 (m, 1H), 2.29-2.18 (m, 2H), 2.16-2.00 (m, 5H), 1.95-1.83 (m, 1H), 1.78-1.62 (m, 2H), 1.38-1.16 (m, 5H), 0.99-0.94 (m, 2H), 0.80 (d, J=6.6 Hz, 2H), 0.69 (d, J=6.5 Hz, 1H), 0.56 (d, J=6.8 Hz, 1H).

### 8. (2S,4R)—N-[[2-[2-[2-[2-[2-[2-[[1-[(2R)-2-[6-chloro-8-fluoro-7-(3- hydroxy-1-naphthyl)-4-(4-prop-2-enoylpiperazin-1-yl)quinazolin-2-yl]oxypropyl]-4-piperidyl]oxy]ethoxy]ethoxy]ethoxy]ethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

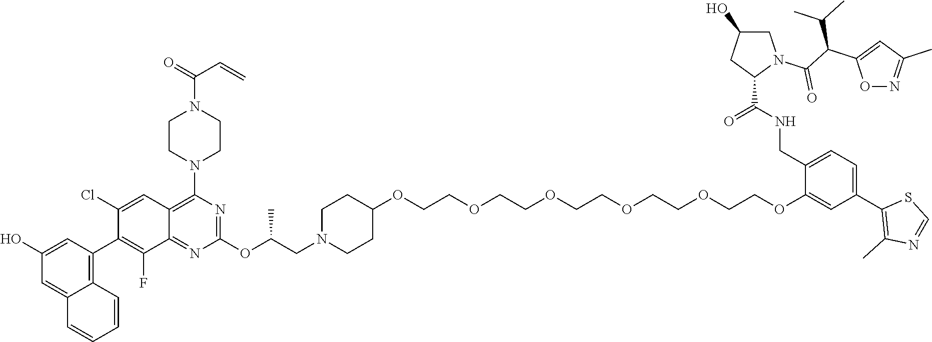 [SMILES: C=CC(=O)N1CCN(c2nc(O[C@H](C)CN3CCC(OCCOCCOCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

(formic acid, white solid). LC/MS (ESI) m/z: 662.0 [M/2+H]⁺. ¹H-NMR (400 MHz, DMSO-d₆) δ 10.00 (s, 1H), 8.97 (s, 1H), 8.62-8.28 (m, 1H), 8.18-7.94 (m, 1H), 7.80 (d, J=8.4 Hz, 1H), 7.47-7.39 (m, 1H), 7.38-7.14 (m, 4H), 7.11-6.95 (m, 3H), 6.83 (dd, J=10.6, 16.7 Hz, 1H), 6.27-5.89 (m, 2H), 5.80-5.65 (m, 1H), 5.48-5.31 (m, 1H), 5.09 (br d, J=1.3 Hz, 1H), 4.48-4.11 (m, 6H), 3.96-3.71 (m, 11H), 3.63-3.38 (m, 18H), 3.23-3.17 (m, 1H), 2.90-2.71 (m, 2H), 2.47-2.42 (m, 4H), 2.30-2.09 (m, 6H), 2.02 (br d, J=8.8 Hz, 1H), 1.97-1.85 (m, 1H), 1.72 (br s, 2H), 1.39-1.17 (m, 5H), 1.00-0.87 (m, 3H), 0.76 (br d, J=6.6 Hz, 3H).

### Exemplary Synthesis of (2S,4R)-1-[(2S)-2-[[2-[4-[1-[(2R)-2-[6-chloro-8-fluoro-7-(3-hydroxy-1-naphthyl)-4-(4-prop-2-enoylpiperazin-1-yl)quinazolin-2-yl]oxypropyl]azetidin-3-yl]oxy-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 3-(4-pyridyloxy) azetidine-1-carboxylate

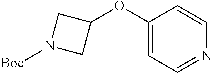 [SMILES: CC(C)(C)OC(=O)N1CC(Oc2ccncc2)C1]

To a solution of tert-butyl 3-hydroxyazetidine-1-carboxylate (10 g, 57.73 mmol, 1 eq) and pyridin-4-ol (8.24 g, 86.60 mmol, 1.5 eq) in THF (100 mL) at 0° C. were added triphenylphosphine (18.17 g, 69.28 mmol, 1.2 eq) and diisopropyl azodicarboxylate (14 g, 69.28 mmol, 13 mL, 1.2 eq), and the reaction mixture was stirred at 40° C. for 12 hours. The reaction mixture was diluted with water (200 mL) and extracted with EtOAc (3×200 mL). The combined organic extracts were washed with brine (3×200 mL), dried over anhydrous Na₂SO₄, filtered, and concentrated. The resulting residue was purified by prep-HPLC (5-35% CH₃CN in water (0.225% formic acid)) to afford tert-butyl 3-(4-pyridyloxy) azetidine-1-carboxylate (3.3 g, 13.18 mmol, 22% yield) as a white solid. LC/MS (ESI) m/z: 250.9 [M+H]⁺. ¹H-NMR (400 MHz, DMSO-d₆) δ 8.50-8.31 (m, 2H), 6.97-6.80 (m, 2H), 5.19-4.98 (m, 1H), 4.43-4.23 (m, 2H), 3.93-3.65 (m, 2H), 1.38 (s, 9H).

### Step 2: Preparation of tert-butyl 3-(1-benzylpyridin-1-ium-4-yl)oxyazetidine-1-carboxylate

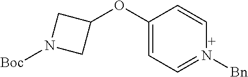

To a solution of tert-butyl 3-(4-pyridyloxy)azetidine-1-carboxylate (3.1 g, 12.39 mmol, 1 eq) in toluene (40 mL) was added benzyl bromide (2.12 g, 12.39 mmol, 1.47 mL, 1 eq), and the reaction mixture was stirred at 80° C. for 12 hours. The mixture was concentrated under reduced pressure, and the resulting crude product was triturated with EtOAc (20 mL) to afford tert-butyl 3-(1-benzylpyridin-1-ium-4-yl)oxyazetidine-1-carboxylate (4 g, 11.72 mmol, 94% yield) as a white solid. LC/MS (ESI) m/z: 340.9 [M+H]⁺.

### Step 3: Preparation of tert-butyl 3-[(1-benzyl-3,6-dihydro-2H-pyridin-4-yl)oxy]azetidine-1-carboxylate

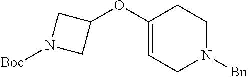 [SMILES: CC(C)(C)OC(=O)N1CC(OC2=CCN(Cc3ccccc3)CC2)C1]

To a solution of tert-butyl 3-(1-benzylpyridin-1-ium-4-yl)oxyazetidine-1-carboxylate (4 g, 11.72 mmol, 1 eq) in ethanol (80 mL) at 0° C. was added NaBH₄ (2.66 g, 70.29 mmol, 6 eq), and the reaction mixture was stirred at 15° C. for 12 hours. The reaction mixture was diluted with water (50 mL) and extracted with EtOAc (3×50 mL). The combined organic layers were washed with brine (3×50 mL), dried over anhydrous Na₂SO₄, filtered, and concentrated under reduced pressure. The resulting residue was purified by prep-HPLC (20-50% CH₃CN in water (0.225% formic acid)) to afford tert-butyl 3-[(1-benzyl-3,6-dihydro-2H-pyridin-4-yl)oxy]azetidine-1-carboxylate (2.5 g, 7.26 mmol, 61% yield) as a colorless oil. LC/MS (ESI) m/z: 345.0 [M+H]⁺.

### Step 4: Preparation of tert-butyl 3-(4-piperidyloxy)azetidine-1-carboxylate

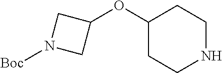 [SMILES: CC(C)(C)OC(=O)N1CC(OC2CCNCC2)C1]

To a solution of tert-butyl 3-[(1-benzyl-3,6-dihydro-2H-pyridin-4-yl)oxy]azetidine-1-carboxylate (2.3 g, 6.68 mmol, 1 eq) in CH₃OH (10 mL) was added Pd/C (500 mg, 10% purity), and the reaction mixture was stirred at 40° C. for 12 hours under hydrogen (15 psi). The reaction mixture was filtered, and the filtrate was concentrated under reduced pressure to afford tert-butyl 3-(4-piperidyloxy)azetidine-1-carboxylate (1.5 g, 5.85 mmol, 87% yield) as a colorless oil. LC/MS (ESI) m/z: 257.0 [M+H]⁺.

### Step 5: Preparation of tert-butyl 3-[[1-(2-ethoxy-2-oxo-ethyl)-4-piperidyl]oxy]azetidine-1-carboxylate

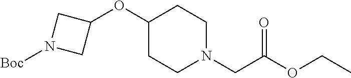 [SMILES: CCOC(=O)CN1CCC(OC2CN(C(=O)OC(C)(C)C)C2)CC1]

To a solution of tert-butyl 3-(4-piperidyloxy)azetidine-1-carboxylate (150 mg, 0.59 mmol, 1 eq) in CH₃CN (5 mL) at 0° C. were added K₂CO₃ (162 mg, 1.17 mmol, 2 eq) and ethyl 2-bromoacetate (98 mg, 0.59 mmol, 0.06 mL, 1 eq), and the reaction mixture was stirred at 25° C. for 1 hour. The mixture was filtered, and the filtrate was poured onto saturated aqueous NH₄Cl (3 mL). The resulting mixture was extracted with EtOAc (2×10 mL), and the combined organic extracts were washed with brine (2×15 mL), dried over Na₂SO₄, filtered, and concentrated. The resulting residue was purified by column chromatography on SiO₂ (2-4% EtOAc in petroleum ether) to afford tert-butyl 3-[[1-(2-ethoxy-2-oxo-ethyl)-4-piperidyl]oxy]azetidine-1-carboxylate (80 mg, 0.23 mmol, 40% yield) as a yellow oil.

### Step 6: Preparation of 2-[4-(1-tert-butoxycarbonylazetidin-3-yl)oxy-1-piperidyl]acetic Acid

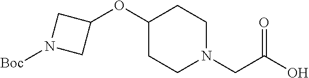 [SMILES: CC(C)(C)OC(=O)N1CC(OC2CCN(CC(=O)O)CC2)C1]

To a solution of tert-butyl 3-[[1-(2-ethoxy-2-oxo-ethyl)-4-piperidyl]oxy]azetidine-1-carboxylate (80 mg, 0.23 mmol, 1 eq) in water (0.5 mL), CH₃OH (0.5 mL) and THF (0.5 mL) was added LiOH (29 mg, 0.70 mmol, 3 eq), and the reaction mixture was stirred at 25° C. for 1 hour. Aqueous HCl (1N) was then added to adjust pH (7-8), and the resulting mixture was concentrated to afford crude 2-[4-(1-tert-butoxycarbonylazetidin-3-yl)oxy-1-piperidyl]acetic acid (73 mg, 0.23 mmol, 99% yield) as a colorless oil.

### Step 7: Preparation of tert-butyl 3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]azetidine-1-carboxylate

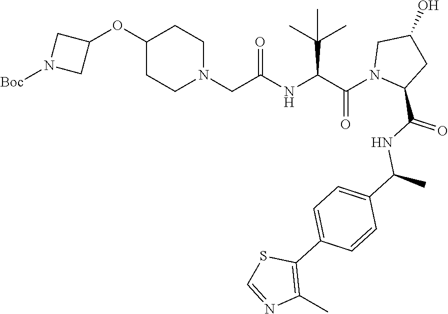 [SMILES: Cc1ncsc1-c1ccc([C@H](C)NC(=O)[C@@H]2C[C@@H](O)CN2C(=O)[C@@H](NC(=O)CN2CCC(OC3CN(C(=O)OC(C)(C)C)C3)CC2)C(C)(C)C)cc1]

To a solution of 2-[4-(1-tert-butoxycarbonylazetidin-3-yl)oxy-1-piperidyl]acetic acid (73 mg, 0.23 mmol, 1 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (112 mg, 0.23 mmol, 1 eq, hydrochloride) in DMF (3 mL) were added HATU (177 mg, 0.46 mmol, 2 eq) and diisopropylethylamine (90 mg, 0.70 mmol, 0.12 mL, 3 eq), and the reaction mixture was stirred at 25° C. for 2 hours. The reaction mixture was diluted with water (30 mL) and extracted with EtOAc (2×20 mL). The combined organic extracts were washed with brine (30 mL), dried over Na₂SO₄, filtered, and concentrated under reduced pressure. The resulting residue was purified by prep-TLC on SiO₂ (CH₂Cl₂:CH₃OH=9:1) to afford tert-butyl 3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]azetidine-1-carboxylate (64 mg, 0.08 mmol, 33% yield, 90% purity) as a colorless oil. LC/MS (ESI) m/z: 741.1 [M+H]⁺.

### Step 8: Preparation of (2S,4R)-1-[(2S)-2-[[2-[4-(azetidin-3-yloxy)-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

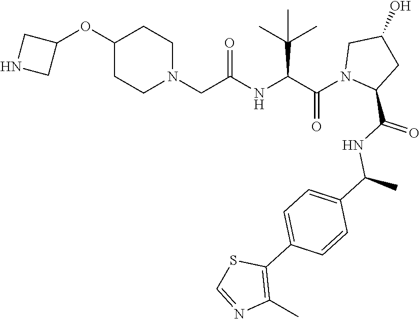 [SMILES: Cc1ncsc1-c1ccc([C@H](C)NC(=O)[C@@H]2C[C@@H](O)CN2C(=O)[C@@H](NC(=O)CN2CCC(OC3CNC3)CC2)C(C)(C)C)cc1]

To a solution of tert-butyl 3-[[1-[2-[[(1S)-1-[(2S,4R)-4-hydroxy-2-[[(1S)-1-[4-(4-methylthiazol- 5-yl)phenyl]ethyl]carbamoyl]pyrrolidine-1-carbonyl]-2,2-dimethyl-propyl]amino]-2-oxo-ethyl]-4-piperidyl]oxy]azetidine-1-carboxylate (104 mg, 0.14 mmol, 1 eq) in CH₂Cl₂ (7 mL) was added trifluoroacetic acid (1.54 g, 13.51 mmol, 1 mL, 96.22 eq), and the reaction mixture was stirred at 25° C. for 1 hour. The mixture was concentrated under reduced pressure to afford (2S,4R)-1-[(2S)-2-[[2-[4-(azetidin-3-yloxy)-1-piperidyl]acetyl]amino]-3,3-dimeth-yl-butanoyl]-4-hydroxy-N-[(1S)-1-[4-(4-methylthiazol-5-yl)phenyl]ethyl]pyrrolidine-2-carboxamide (105 mg, 0.14 mmol, 99% yield, TFA salt) as a yellow solid.

### Step 9: Preparation of tert-butyl 4-[6-chloro-8-fluoro-2-[(1R)-2-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]azetidin-1-yl]-1-methyl-ethoxy]-7-(3-hydroxy-1-naphthyl)quinazolin-4-yl]piperazine-1-carboxylate

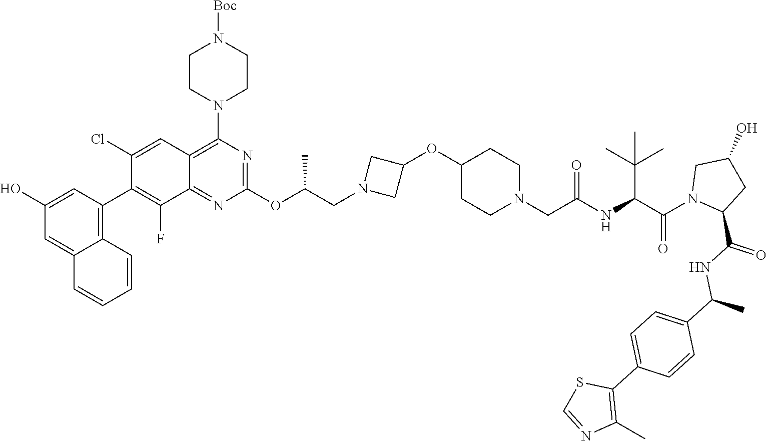 [SMILES: Cc1ncsc1-c1ccc([C@H](C)NC(=O)[C@@H]2C[C@@H](O)CN2C(=O)[C@@H](NC(=O)CN2CCC(OC3CN(C[C@@H](C)Oc4nc(N5CCN(C(=O)OC(C)(C)C)CC5)c5cc(Cl)c(-c6cc(O)cc7ccccc67)c(F)c5n4)C3)CC2)C(C)(C)C)cc1]

To a solution of (2S,4R)-1-[(2S)-2-[[2-[4-(azetidin-3-yloxy)-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (105 mg, 0.14 mmol, 1 eq, TFA salt) in CH₃OH (3 mL) at 0° C. was added NaOAc (57 mg, 0.70 mmol, 5 eq), and the resulting mixture was stirred for 0.5 h. tert-Butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (80 mg, 0.14 mmol, 1 eq) and acetic acid (0.84 mg, 0.01 mmol, 7.96e-1 uL, 0.1 eq) in CH₂Cl₂ (1 mL) were then added, and the resulting mixture was stirred at 25° C. for 0.5 hours. NaBH₃CN (17 mg, 0.28 mmol, 2 eq) was added, and the reaction mixture was stirred at 25° C. for 11 hours. The reaction mixture was quenched with water (30 mL) and extracted with CH₂Cl₂ (2×20 mL). The combined organic extracts were washed with brine (30 mL), dried over Na₂SO₄, filtered, and concentrated under reduced pressure. The resulting residue was purified by prep-TLC on SiO₂ (CH₂Cl₂:CH₃OH=8:1) to afford tert-butyl 4-[6-chloro-8-fluoro-2-[(1R)-2-[3-[[1-[2-[[(1S)-1-[(2S,4R)-4-hydroxy-2-[[(1S)-1-[4-(4- methylthiazol-5-yl)phenyl]ethyl]carbamoyl]pyrrolidine-1-carbonyl]-2,2-dimethyl-propyl]amino]-2-oxo-ethyl]-4-piperidyl]oxy]azetidin-1-yl]-1-methyl-ethoxy]-7-(3-hydroxy-1-naphthyl)quinazolin-4-yl]piperazine-1-carboxylate (100 mg, 0.08 mmol, 59% yield) as a light yellow oil. LC/MS (ESI) m/z: 1205.2 [M+H]⁺.

### Step 10: Preparation of (2S,4R)-1-[(2S)-2-[[2-[4-[1-[(2R)-2-[6-chloro-8-fluoro-7-(3- hydroxy-1-naphthyl)-4-piperazin-1-yl-quinazolin-2-yl]oxypropyl]azetidin-3-yl]oxy-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

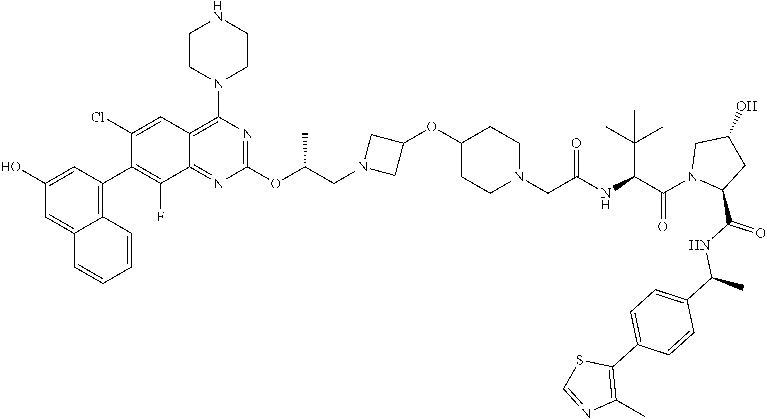 [SMILES: Cc1ncsc1-c1ccc([C@H](C)NC(=O)[C@@H]2C[C@@H](O)CN2C(=O)[C@@H](NC(=O)CN2CCC(OC3CN(C[C@@H](C)Oc4nc(N5CCNCC5)c5cc(Cl)c(-c6cc(O)cc7ccccc67)c(F)c5n4)C3)CC2)C(C)(C)C)cc1]

To a solution of tert-butyl 4-[6-chloro-8-fluoro-2-[(1R)-2-[3-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]oxy]azetidin-1-yl]-1-methyl-ethoxy]-7-(3-hydroxy-1-naphthyl)quinazolin-4-yl]piperazine-1-carboxylate (100 mg, 0.08 mmol, 1 eq) in CH₂Cl₂ (7 mL) was added trifluoroacetic acid (1.54 g, 13.51 mmol, 1 mL, 162.87 eq), and the reaction mixture was stirred at 25° C. for 1 hour. The mixture was concentrated under reduced pressure to give (2S,4R)-1-[(2S)-2-[[2-[4-[1-[(2R)-2-[6-chloro-8-fluoro-7-(3-hydroxy-1-naphthyl)-4-piperazin-1-yl-quinazolin-2-yl]oxypropyl]azetidin-3-yl]oxy-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (101 mg, 0.08 mmol, 99% yield, TFA salt) as a yellow solid.

### Step 11: Preparation of (2S,4R)-1-[(2S)-2-[[2-[4-[1-[(2R)-2-[6-chloro-8-fluoro-7-(3- hydroxy-1-naphthyl)-4-(4-prop-2-enoylpiperazin-1-yl)quinazolin-2-yl]oxypropyl]azetidin-3-yl]oxy-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

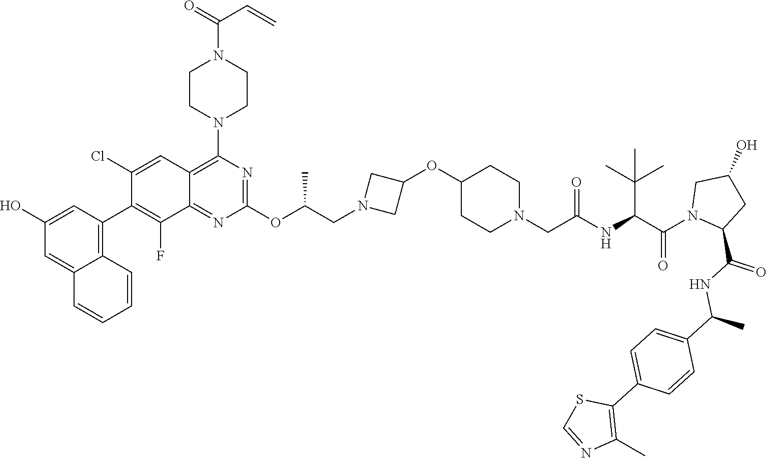 [SMILES: C=CC(=O)N1CCN(c2nc(O[C@H](C)CN3CC(OC4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1]

To a solution of (2S,4R)-1-[(2S)-2-[[2-[4-[1-[(2R)-2-[6-chloro-8-fluoro-7-(3-hydroxy-1-naphthyl)-4-piperazin-1-yl-quinazolin-2-yl]oxypropyl]azetidin-3-yl]oxy-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (100 mg, 0.08 mmol, 1 eq, TFA salt) and 2,6-Lutidine (351 mg, 3.28 mmol, 0.38 mL, 40 eq) in CH₂Cl₂ (3 mL) at −78° C. was added prop-2-enoyl chloride (6.68 mg, 73.78 umol, 0.006 mL, 0.9 eq) in CH₂Cl₂ (1 mL) dropwise, and the reaction mixture was stirred at −78° C. for 30 minutes. The reaction mixture was diluted by water (30 mL) and extracted with CH₂Cl₂ (2×20 mL). The combined organic extracts were washed with brine (30 mL), dried over Na₂SO₄, filtered, and concentrated under reduced pressure. The resulting residue was purified by semi-preparative reverse phase HPLC (18-48% CH₃CN in water (0.225% formic acid)) to afford (2S,4R)-1-[(2S)-2-[[2-[4-[1-[(2R)-2-[6-chloro-8-fluoro-7-(3-hydroxy-1-naphthyl)-4-(4-prop-2-enoylpiperazin-1-yl)quinazolin-2-yl]oxypropyl]azetidin-3-yl]oxy-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (4.2 mg, 0.004 mmol, 4% yield, 100% purity, formic acid salt) as a white solid. ¹H-NMR (400 MHz, DMSO-d₆) δ 10.04 (s, 1H), 8.98 (s, 1H), 8.43 (d, J=7.5 Hz, 1H), 8.15-8.01 (m, 1H), 7.86-7.65 (m, 2H), 7.46-7.42 (m, 2H), 7.37-7.35 (m, 1H), 7.29 (d, J=2.3 Hz, 1H), 7.26-7.17 (m, 2H), 7.07 (dd, J=2.4, 3.4 Hz, 1H), 6.90-6.78 (m, 1H), 6.18 (dd, J=2.2, 16.7 Hz, 1H), 5.79-5.70 (m, 1H), 5.27-5.13 (m, 1H), 4.89 (quin, J=7.0 Hz, 1H), 4.49-4.36 (m, 2H), 4.28 (s, 1H), 4.15-4.05 (m, 1H), 3.95-3.77 (m, 6H), 3.62-3.58 (m, 6H), 3.47-3.43 (m, 3H), 3.17 (d, J=5.3 Hz, 1H), 3.03-2.82 (m, 1H), 2.61 (s, 1H), 2.45 (s, 3H), 2.21-2.01 (m, 3H), 1.77-1.74 (m, 6H), 1.45 (d, J=6.8 Hz, 1H), 1.39-1.34 (m, 3H), 1.30 (d, J=6.1 Hz, 2H), 1.23 (s, 2H), 1.05 (t, J=7.0 Hz, 3H), 0.92 (s, 9H).

### Exemplary Synthesis of (2S,4R)-1-[(2S)-2-[[2-[3-[3-[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-[2-[2-(2-ethoxy-2-oxo-ethoxy)ethoxy]ethoxy]piperidine-1-carboxylate

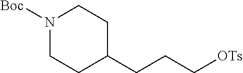 [SMILES: Cc1ccc(S(=O)(=O)OCCCC2CCN(C(=O)OC(C)(C)C)CC2)cc1]

To a mixture of tert-butyl 4-(3-hydroxypropyl)piperidine-1-carboxylate (1 g, 4.11 mmol, 1 eq) and 4-methylbenzene-1-sulfonyl chloride (940 mg, 4.93 mmol, 1.2 eq) in CH₂Cl₂ (10 mL) was added triethylamine (1.25 g, 12.33 mmol, 1.72 mL, 3 eq), and the reaction mixture was stirred at 25° C. for 15 hours under nitrogen atmosphere. Purification by flash chromatography on SiO₂ (0-25% EtOAc in petroleum ether) afforded tert-butyl 4-[3-(p-tolylsulfonyloxy)propyl]piperidine-1-carboxylate (1.45 g, 3.65 mmol, 88% yield) as a colorless oil. ¹H-NMR (400 MHz, CDCl₃) δ 8.10 (d, J=8.4 Hz, 2H), 7.37 (d, J=8.0 Hz, 2H), 4.05-4.02 (m, 4H), 2.64 (t, J=12.0 Hz, 2H), 2.47 (s, 3H), 1.66-1.60 (m, 2H), 1.59 (d, J=12.8 Hz, 2H), 1.46 (s, 9H), 1.30-1.25 (m, 3H), 1.10-1.00 (m, 2H).

### Step 2: Preparation of tert-butyl 4-[3-(1-benzyloxycarbonylazetidin-3-yl)oxypropyl]piperidine-1-carboxylate

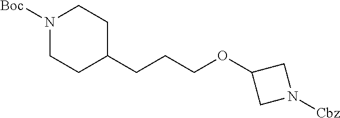 [SMILES: CC(C)(C)OC(=O)N1CCC(CCCOC2CN(C(=O)OCc3ccccc3)C2)CC1]

To a solution of tert-butyl 4-[2 A solution of benzyl 3-hydroxyazetidine-1-carboxylate (313 mg, 1.51 mmol, 2 eq) in DMF (5 mL) at 0° C. was added NaH (60.37 mg, 1.51 mmol, 60% purity, 2 eq), and the reaction mixture was stirred at 25° C. for 1 hour. tert-Butyl 4-[3-(p-tolylsulfonyloxy)propyl]piperidine-1-carboxylate (300 mg, 0.75 mmol, 1 eq) was then added, and the reaction mixture was stirred at 25° C. for 14 hours. The reaction was quenched with water (30 mL), and the resulting mixture was extracted with EtOAc (30 mL). The organic extract was washed with brine (30 mL), dried over Na₂SO₄, and concentrated. The resulting residue was purified by column chromatography on SiO₂ (0-20% EtOAc in petroleum ether) to get tert-butyl 4-[3-(1-benzyloxycarbonylazetidin-3-yl)oxypropyl]piperidine-1-carboxylate (280 mg, 0.65 mmol, 85% yield) as a colorless oil. LC/MS (ESI) m/z: 455.2 [M+Na]⁺. ¹H-NMR (400 MHz, CDCl₃) δ 7.41-7.34 (m, 1H), 7.35-7.30 (m, 1H), 5.15-5.09 (m, 2H), 4.27-4.22 (m, 1H), 4.20-4.03 (m, 4H), 3.92 (dd, J=4.2, 9.9 Hz, 2H), 3.36 (t, J=6.5 Hz, 2H), 2.78-2.54 (m, 2H), 1.69-1.60 (m, 4H), 1.48 (s, 9H), 1.38 (dtd, J=3.6, 7.1, 10.5 Hz, 1H), 1.34-1.27 (m, 2H), 1.17-1.03 (m, 2H).

### Step 3: Preparation of tert-butyl 4-[3-(azetidin-3-yloxy)propyl]piperidine-1-carboxylate

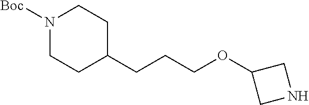 [SMILES: CC(C)(C)OC(=O)N1CCC(CCCOC2CNC2)CC1]

To a solution of tert-butyl 4-[3-(1-benzyloxycarbonylazetidin-3-yl)oxypropyl]piperidine-1-carboxylate (280 mg, 0.65 mmol, 1 eq) in trifluoroethanol (30 mL) was added Pd/C (50 mg, 5% purity) under nitrogen atmosphere, and the reaction mixture was degassed with H₂ (3×) and then stirred at 25° C. for 16 hours. The reaction mixture was filtered and the solid was washed with CH₃OH (20 mL). The filtrate was concentrated to get tert-butyl 4-[3-(azetidin-3-yloxy)propyl]piperidine-1-carboxylate (193 mg, 646.74 umol, 99.91% yield) as colorless gum. ¹H-NMR (400 MHz, CDCl₃) δ 4.29 (quin, J=6.3 Hz, 1H), 4.15-4.00 (m, 2H), 3.72-3.65 (m, 1H), 3.63-3.56 (m, 2H), 3.32 (t, J=6.6 Hz, 2H), 2.67 (br t, J=11.8 Hz, 2H), 1.65 (br d, J=12.6 Hz, 2H), 1.60-1.53 (m, 2H), 1.46 (s, 9H), 1.37 (ddd, J=3.8, 7.2, 14.1 Hz, 1H), 1.33-1.24 (m, 2H), 1.14-1.01 (m, 2H).

### Step 4: Preparation of tert-butyl 4-[3-[1-(2-ethoxy-2-oxo-ethyl)azetidin-3-yl]oxypropyl]piperidine-1-carboxylate

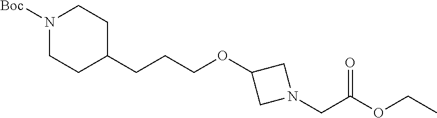 [SMILES: CCOC(=O)CN1CC(OCCCC2CCN(C(=O)OC(C)(C)C)CC2)C1]

To a mixture of 0.-butyl 4-[3-(azetidin-3-yloxy)propyl]piperidine-1-carboxylate (190 mg, 0.64 mmol, 1 eq) and K₂CO₃ (176 mg, 1.27 mmol, 2 eq) in CH₃CN (4 mL) at 0° C. was added ethyl 2-chloroacetate (78 mg, 0.64 mmol, 1 eq), and the reaction mixture was stirred at 25° C. for 16 hours. The reaction mixture was concentrated, and the resulting residue was purified by silica gel chromatography (0-2% CH₃OH in CH₂Cl₂) to get tert-butyl 4-[3-[1-(2-ethoxy-2-oxo-ethyl)azetidin-3-yl]oxypropyl]piperidine-1-carboxylate (128 mg, 0.33 mmol, 52% yield) as a colorless gum. ¹H-NMR (400 MHz, CDCl₃) δ 4.22-3.85 (m, 5H), 3.82-3.76 (m, 1H), 3.66-3.28 (m, 4H), 3.05-2.93 (m, 1H), 2.90-2.58 (m, 3H), 1.69-1.62 (m, 2H), 1.59-1.52 (m, 2H), 1.46 (s, 9H), 1.41-1.32 (m, 1H), 1.31-1.23 (m, 5H), 1.14-0.97 (m, 2H).

### Step 5: Preparation of 2-[3-[3-(1-tert-butoxycarbonyl-4-piperidyl)propoxy]azetidin-1-yl]acetic Acid

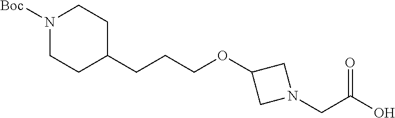 [SMILES: CC(C)(C)OC(=O)N1CCC(CCCOC2CN(CC(=O)O)C2)CC1]

To a solution of tert-butyl 4-[3-[1-(2-ethoxy-2-oxo-ethyl)azetidin-3-yl]oxypropyl]piperidine-1-carboxylate (128 mg, 0.33 mmol, 1 eq) in CH₃OH (0.5 mL) and THF (1 mL) were added LiOH monohydrate (42 mg, 1.00 mmol, 3 eq) and water (1 mL), and the reaction mixture was stirred at 25° C. for 1 hour. The reaction was diluted with THF (25 mL) and the pH was adjusted to 8 by adding 1N sulfuric acid. EtOAc (20 mL) was added, and the resulting mixture was dried over Na₂SO₄ concentrated to get crude 2-[3-[3-(1-tert-butoxycarbonyl-4-piperidyl)propoxy]azetidin-1-yl]acetic acid (120 mg) as colorless gum. ¹H-NMR (400 MHz, CDCl₃) δ 4.48 (br s, 2H), 4.13-3.96 (m, 3H), 3.78-3.58 (m, 3H), 3.46-3.27 (m, 2H), 2.67 (br s, 2H), 1.71-1.50 (m, 4H), 1.48-1.41 (m, 9H), 1.40-1.32 (m, 1H), 1.29-1.23 (m, 2H), 1.14-0.96 (m, 2H).

### Step 6: Preparation of tert-butyl 4-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]oxypropyl]piperidine-1-carboxylate

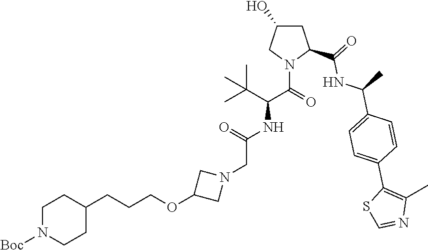 [SMILES: Cc1ncsc1-c1ccc([C@H](C)NC(=O)[C@@H]2C[C@@H](O)CN2C(=O)[C@@H](NC(=O)CN2CC(OCCCC3CCN(C(=O)OC(C)(C)C)CC3)C2)C(C)(C)C)cc1]

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (150 mg, 0.31 mmol, 1 eq, hydrochloride), 2-[3-[3-(1-tert-butoxycarbonyl-4-piperidyl)propoxy]azetidin-1-yl]acetic acid (118 mg, 0.33 mmol, 1.06 eq), hydroxybenzotriazole (63 mg, 0.47 mmol, 1.5 eq), and 1-(3-dimethylaminopropyl)-3-ethylcarbodiimide hydrochloride (90 mg, 0.47 mmol, 1.5 eq) in DMF (3 mL) was added diisopropylethylamine (121 mg, 0.94 mmol, 3 eq), and the reaction mixture was stirred at 25° C. for 14 hours. The reaction mixture was concentrated, and the resulting residue was purified by prep-TLC on SiO₂ (10% CH₃OH in CH₂Cl₂) to get tert-butyl 4-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]oxypropyl]piperidine-1-carboxylate (125 mg, 0.15 mmol, 47% yield, 92% purity) as colorless gum. LC/MS (ESI) m/z: 783.3 [M+H]⁺.

### Step 7: Preparation of (2S,4R)-1-[(2S)-3,3-dimethyl-2-[[2-[3-[3-(4-piperidyl)propoxy]azetidin-1-yl]acetyl]amino]butanoyl]-4-hydroxy-N-[(1S)-1-[4-(4-methylthiazol-5-yl)phenyl]ethyl]pyrrolidine-2-carboxamide

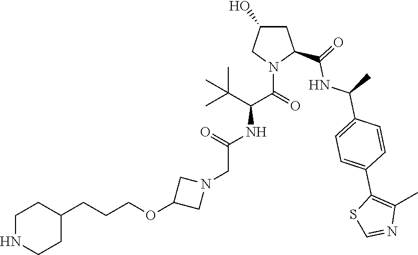 [SMILES: Cc1ncsc1-c1ccc([C@H](C)NC(=O)[C@@H]2C[C@@H](O)CN2C(=O)[C@@H](NC(=O)CN2CC(OCCCC3CCNCC3)C2)C(C)(C)C)cc1]

To a solution of tert-butyl 4-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]oxypropyl]piperidine-1-carboxylate (125 mg, 0.16 mmol, 1 eq) in CH₂Cl₂ (2 mL) was added trifluoroacetic acid (616 mg, 5.40 mmol, 0.4 mL, 33.84 eq), and the reaction mixture was stirred at 25° C. for 1 hour. The reaction mixture was concentrated under vacuum to get (2S,4R)-1-[(2S)-3,3-dimethyl-2-[[2-[3-[3-(4-piperidyl)propoxy]azetidin-1-yl]acetyl]amino]butanoyl]-4-hydroxy-N-[(1S)-1-[4-(4-methylthiazol-5-yl)phenyl]ethyl]pyrrolidine-2-carboxamide (127 mg, 0.16 mmol, 99% yield, TFA salt) as a colorless gum.

### Step 8: Preparation of tert-butyl 4-[6-chloro-8-fluoro-2-[(1R)-2-[4-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]oxypropyl]-1-piperidyl]-1-methyl-ethoxy]-7-(3-hydroxy-1-naphthyl)quinazolin-4-yl]piperazine-1-carboxylate

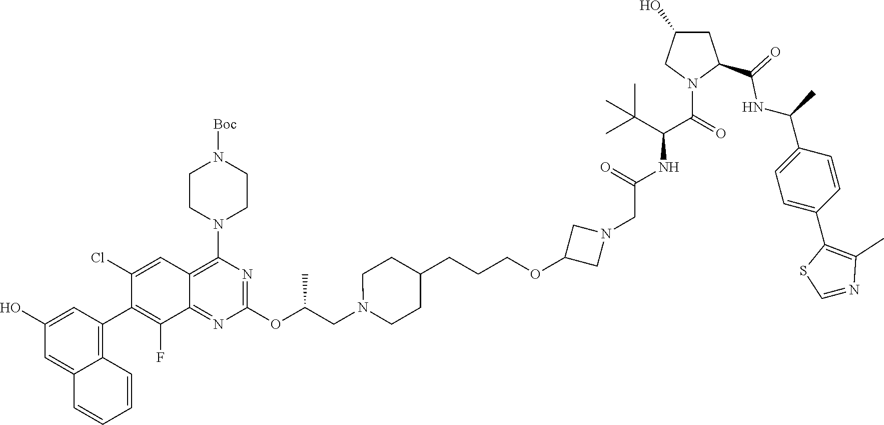 [SMILES: Cc1ncsc1-c1ccc([C@H](C)NC(=O)[C@@H]2C[C@@H](O)CN2C(=O)[C@@H](NC(=O)CN2CC(OCCCC3CCN(C[C@@H](C)Oc4nc(N5CCN(C(=O)OC(C)(C)C)CC5)c5cc(Cl)c(-c6cc(O)cc7ccccc67)c(F)c5n4)CC3)C2)C(C)(C)C)cc1]

To a solution of (2S,4R)-1-[(2S)-3,3-dimethyl-2-[[2-[3-[3-(4-piperidyl)propoxy]azetidin-1-yl]acetyl]amino]butanoyl]-4-hydroxy-N-[(1S)-1-[4-(4-methylthiazol-5-yl)phenyl]ethyl]pyrrolidine-2-carboxamide (127 mg, 0.16 mmol, 1 eq, TFA salt) in CH₂Cl₂ (2 mL) was added NaOAc (39.22 mg, 478.08 umol, 3 eq), and the resulting mixture was stirred at 25° C. for 30 minutes. A solution of tert-butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (97 mg, 0.17 mmol, 1.05 eq) in CH₃OH (2 mL) was then added, and the reaction mixture was cooled to 0° C. NaBH₃CN (20 mg, 0.32 mmol, 2 eq) was added, and the reaction mixture was stirred at 25° C. for 14 hours. The reaction mixture was concentrated, and the resulting residue was purified by prep-TLC on SiO₂ (10% CH₃OH in CH₂Cl₂) to get tert-butyl 4-[6-chloro-8-fluoro-2-[(1R)-2-[4-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]oxypropyl]-1-piperidyl]-1-methyl-ethoxy]-7-(3-hydroxy-1-naphthyl)quinazolin-4-yl]piperazine-1-carboxylate (62 mg, 0.050 mol, 31% yield) as a colorless gum. LC/MS (ESI) m/z: 1247.4. [M+H]⁺.

### Step 9: Preparation of (2S,4R)-1-[(2S)-2-[[2-[3-[3-[1-[(2R)-2-[6-chloro-8-fluoro-7-(3-hydroxy-1-naphthyl)-4-piperazin-1-yl-quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

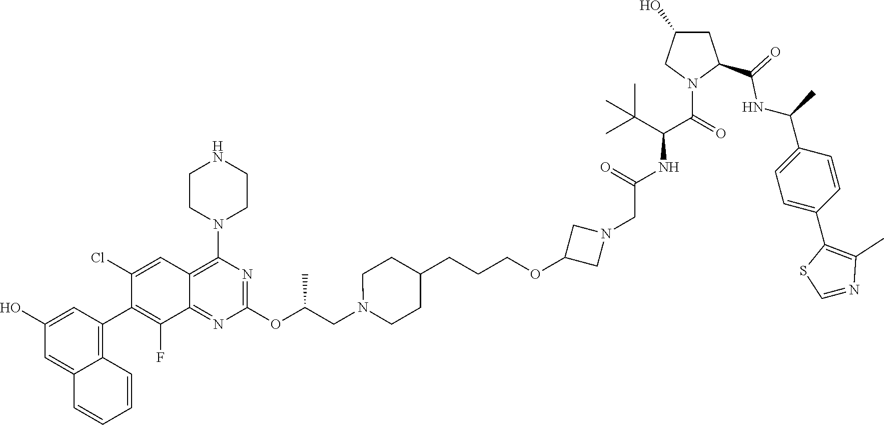 [SMILES: Cc1ncsc1-c1ccc([C@H](C)NC(=O)[C@@H]2C[C@@H](O)CN2C(=O)[C@@H](NC(=O)CN2CC(OCCCC3CCN(C[C@@H](C)Oc4nc(N5CCNCC5)c5cc(Cl)c(-c6cc(O)cc7ccccc67)c(F)c5n4)CC3)C2)C(C)(C)C)cc1]

To a solution of tert-butyl 4-[6-chloro-8-fluoro-2-[(1R)-2-[4-[3-[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]oxypropyl]-1-piperidyl]-1-methyl-ethoxy]-7-(3-hydroxy-1-naphthyl)quinazolin-4-yl]piperazine-1-carboxylate (62 mg, 0.050 mmol, 1 eq) in CH₂Cl₂ (2 mL) was added trifluoroacetic acid (616 mg, 5.40 mmol, 0.4 mL, 108.74 eq), and the reaction mixture was stirred at 25° C. for 1 hour. The reaction mixture was concentrated under vacuum to get (2S,4R)-1-[(2S)-2-[[2-[3-[3-[1-[(2R)-2-[6-chloro-8-fluoro-7-(3- hydroxy-1-naphthyl)-4-piperazin-1-yl-quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (62 mg, 0.049 mmol, 99% yield, TFA salt) as a yellow gum. LC/MS (ESI) m/z: 1148.6. [M+H]⁺.

### Step 10: Preparation of (2S,4R)-1-[(2S)-2-[[2-[3-[3-[1-[(2R)-2-[6-chloro-8-fluoro-7- (3-hydroxy-1-naphthyl)-4-(4-prop-2-enoylpiperazin-1-yl)quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

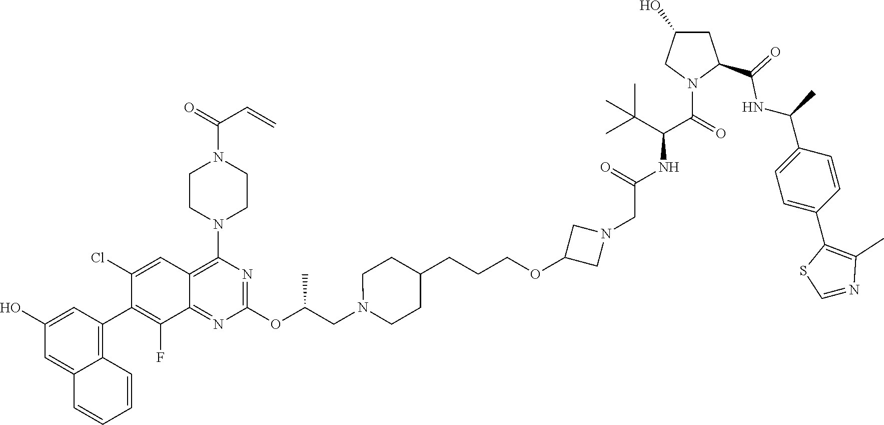 [SMILES: C=CC(=O)N1CCN(c2nc(O[C@H](C)CN3CCC(CCCOC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1]

To a solution of (2S,4R)-1-[(2S)-2-[[2-[3-[3-[1-[(2R)-2-[6-chloro-8-fluoro-7-(3- hydroxy-1-naphthyl)-4-piperazin-1-yl-quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (62 mg, 0.049 mmol, 1 eq, TFA salt) and 2,6-lutidine (105 mg, 0.98 mmol, 20 eq) in CH₂Cl₂ (4 mL) at −78° C. was added a solution of prop-2-enoyl chloride (4.00 mg, 0.044 mmol, 0.9 eq) in CH₂Cl₂ (0.36 mL), and the reaction mixture was stirred at −78° C. for 10 minutes. Water (0.1 mL) and DMF (0.5 mL) were added and the reaction mixture was concentrated. The resulting residue was purified by prep-HPLC (20-50% CH₃CN in water (0.225% formic acid)) to get (2S,4R)-1-[(2S)-2-[[2-[3-[3-[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (8.8 mg, 0.007 mmol, 14% yield, 97% purity) as colorless gum. LC/MS (ESI) m/z: 1201.5 [M+H]⁺. ¹H-NMR (400 MHz, DMSO-d₆) δ 10.01 (s, 1H), 9.00-8.95 (m, 1H), 8.42 (d, J=7.9 Hz, 1H), 8.15 (s, 1H), 8.00 (s, 1H), 7.81 (d, J=8.3 Hz, 1H), 7.52 (br d, J=9.8 Hz, 1H), 7.47-7.40 (m, 4H), 7.39-7.33 (m, 2H), 7.29 (d, J=2.3 Hz, 1H), 7.25-7.17 (m, 2H), 7.06 (dd, J=2.3, 4.9 Hz, 1H), 6.83 (dd, J=10.5, 16.7 Hz, 1H), 6.21-6.15 (m, 1H), 5.78-5.69 (m, 1H), 5.46-5.34 (m, 1H), 5.15-5.08 (m, 1H), 4.98-4.83 (m, 1H), 4.50-4.45 (m, 1H), 4.45-4.39 (m, 1H), 4.30-4.23 (m, 1H), 4.10-4.03 (m, 1H), 3.92 (br s, 4H), 3.88-3.82 (m, 2H), 3.80-3.74 (m, 2H), 3.63-3.49 (m, 5H), 3.24 (br t, J=6.4 Hz, 2H), 3.09 (br d, J=8.4 Hz, 2H), 2.99-2.85 (m, 4H), 2.64-2.59 (m, 1H), 2.45 (s, 3H), 2.43-2.38 (m, 1H), 2.08-1.84 (m, 4H), 1.76 (ddd, J=4.2, 8.6, 12.9 Hz, 1H), 1.57-1.49 (m, 2H), 1.43 (td, J=6.1, 8.1 Hz, 2H), 1.37 (d, J=6.8 Hz, 3H), 1.30 (br d, J=6.2 Hz, 3H), 1.17-1.09 (m, 3H), 0.91 (s, 9H).

The following compounds were prepared in an analogous manner to (2S,4R)-1-[(2S)-2-[[2-[3-[3-[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4R)-1-[(2S)-2-[[2-[6-[[1-[(2R)-2-[6-chloro-8-fluoro-7-(3-hydroxy-1- naphthyl)-4-(4-prop-2-enoylpiperazin-1-yl)quinazolin-2-yl]oxypropyl]-4-piperidyl]methyl]-2,6-diazaspiro[3.3]heptan-2-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

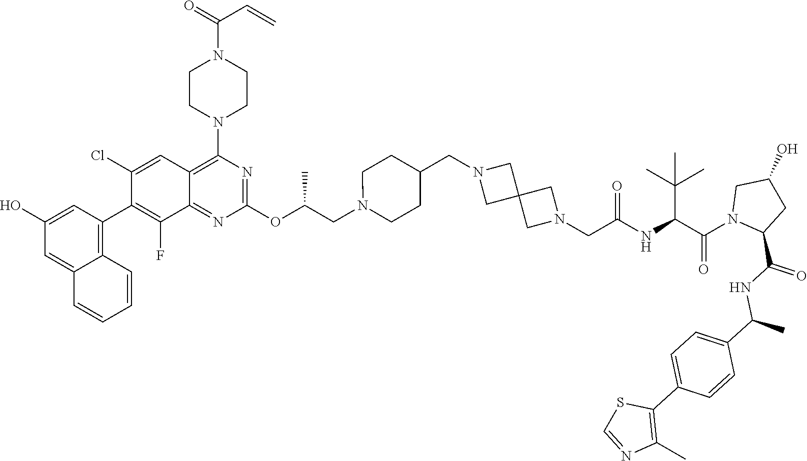 [SMILES: C=CC(=O)N1CCN(c2nc(O[C@H](C)CN3CCC(CN4CC5(CN(CC(=O)N[C@H](C(=O)N6C[C@H](O)C[C@H]6C(=O)N[C@@H](C)c6ccc(-c7scnc7C)cc6)C(C)(C)C)C5)C4)CC3)nc3c(F)c(-c4cc(O)cc5ccccc45)c(Cl)cc23)CC1]

(formic acid salt, white solid). LC/MS (ESI) m/z: 1198.6 [M+H]⁺. ¹H-NMR (400 MHz, DMSO-d₆) δ 10.04 (s, 1H), 8.99 (s, 1H), 8.71-8.40 (m, 1H), 8.17 (s, 1H), 8.01 (s, 1H), 7.82 (d, J=8.4 Hz, 1H), 7.54-7.47 (m, 1H), 7.46-7.41 (m, 3H), 7.40-7.33 (m, 2H), 7.30 (d, J=2.3 Hz, 1H), 7.28-7.18 (m, 2H), 7.08 (dd, J=2.4, 3.7 Hz, 1H), 6.84 (dd, J=10.4, 16.7 Hz, 1H), 6.19 (dd, J=2.3, 16.6 Hz, 1H), 5.79-5.73 (m, 1H), 5.46-5.34 (m, 1H), 5.29-4.96 (m, 1H), 4.95-4.84 (m, 1H), 4.57-4.35 (m, 2H), 4.32-4.21 (m, 1H), 3.97-3.89 (m, 4H), 3.88-3.76 (m, 4H), 3.62-3.51 (m, 3H), 3.34 (br d, J=6.8 Hz, 4H), 3.13-3.01 (m, 3H), 3.01-2.84 (m, 3H), 2.63 (br dd, J=6.8, 12.6 Hz, 1H), 2.46 (s, 3H), 2.39 (br dd, J=3.0, 11.6 Hz, 1H), 2.28 (br t, J=6.4 Hz, 2H), 2.08-1.87 (m, 3H), 1.77 (ddd, J=4.4, 8.8, 12.8 Hz, 1H), 1.57-1.47 (m, 2H), 1.38 (d, J=6.9 Hz, 3H), 1.31 (br d, J=6.1 Hz, 3H), 1.24-1.12 (m, 1H), 0.92 (s, 12H).

### 2. (2S,4R)-1-[(2S)-2-[[2-[3-[2-[[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]oxy]ethoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

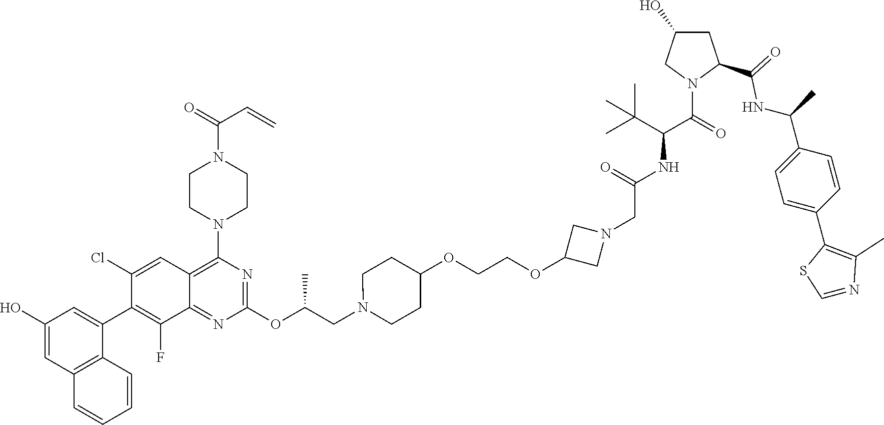 [SMILES: C=CC(=O)N1CCN(c2nc(O[C@H](C)CN3CCC(OCCOC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1]

(formic acid salt, white solid). LC/MS (ESI) m/z: 1203.06[M+H]⁺. ¹H-NMR (400 MHz, DMSO-d₆) δ 10.75-9.42 (m, 1H), 9.05-8.90 (m, 1H), 8.44 (d, J=7.6 Hz, 1H), 8.25 (s, 1H), 8.01 (s, 1H), 7.82 (d, J=8.0 Hz, 1H), 7.51 (d, J=9.6 Hz, 1H), 7.48-7.42 (m, 3H), 7.40-7.35 (m, 2H), 7.30 (d, J=2.0 Hz, 1H), 7.25-7.18 (m, 2H), 7.08 (dd, J=2.4, 4.8 Hz, 1H), 6.84 (dd, J=10.4, 16.4 Hz, 1H), 6.19 (dd, J=2.0, 16.8 Hz, 1H), 5.79-5.71 (m, 1H), 5.45-5.34 (m, 1H), 4.95-4.86 (m, 1H), 4.51-4.40 (m, 2H), 4.28 (br s, 1H), 4.15-4.09 (m, 1H), 3.98-3.77 (s, 9H), 3.64-3.51 (m, 3H), 3.23-3.19 (m, 2H), 3.16-3.03 (m, 4H), 3.00-2.90 (m, 2H), 2.88-2.71 (m, 3H), 2.66-2.59 (m, 1H), 2.46 (s, 3H), 2.42-2.35 (m, 2H), 2.18-2.01 (m, 4H), 1.81-1.70 (m, 3H), 1.38 (d, J=7.2 Hz, 3H), 1.31 (br dd, J=2.0, 6.0 Hz, 4H), 0.91 (s, 9H).

### 3. (2S,4R)-1-[(2S)-2-[[2-[4-[3-[4-[(2R)-2-[6-chloro-8-fluoro-7-(3-hydroxy-1-naphthyl)-4-(4-prop-2-enoylpiperazin-1-yl)quinazolin-2-yl]oxypropyl]piperazin-1-yl]propyl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

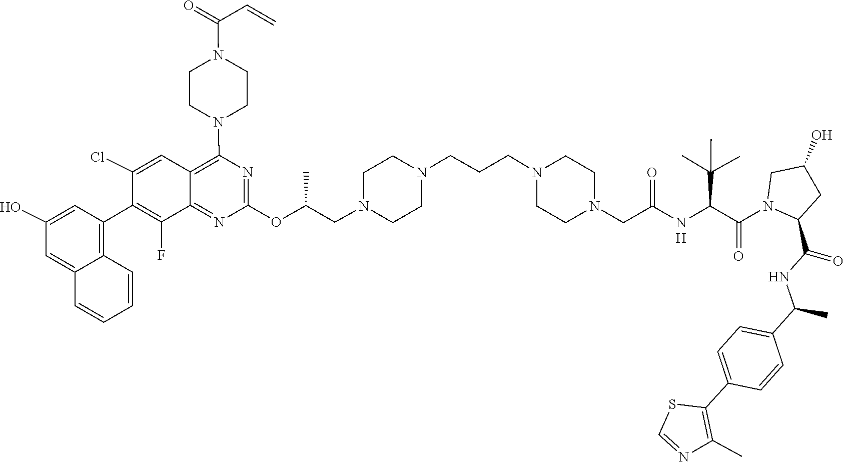

(formic acid salt, white solid). LC/MS (ESI) m/z: 1216.6 [M+H]⁺. ¹H-NMR (400 MHz, DMSO-d₆) δ 10.04 (s, 1H), 8.98 (s, 1H), 8.42 (d, J=7.7 Hz, 1H), 8.02 (s, 1H), 7.81 (d, J=8.4 Hz, 1H), 7.70 (d, J=8.2 Hz, 1H), 7.48-7.41 (m, 3H), 7.39-7.26 (m, 3H), 7.26-7.17 (m, 2H), 7.07 (dd, J=2.4, 5.3 Hz, 1H), 6.83 (dd, J=10.5, 16.7 Hz, 1H), 6.18 (dd, J=2.3, 16.7 Hz, 1H), 5.80-5.70 (m, 1H), 5.41 (s, 1H), 5.15-4.83 (m, 2H), 4.53-4.24 (m, 3H), 3.99-3.72 (m, 9H), 3.62-3.53 (m, 1H), 3.13-2.90 (m, 4H), 2.83-2.53 (m, 16H), 2.45 (s, 8H), 2.09-1.99 (m, 1H), 1.81-1.60 (m, 3H), 1.35 (dd, J=6.5, 19.7 Hz, 5H), 0.93 (s, 9H).

### 4. (2S,4R)-1-[(2S)-2-[[2-[4-[3-[1-[(2R)-2-[6-chloro-8-fluoro-7-(3-hydroxy-1-naphthyl)-4-(4-prop-2-enoylpiperazin-1-yl)quinazolin-2-yl]oxypropyl]-4-piperidyl]propyl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

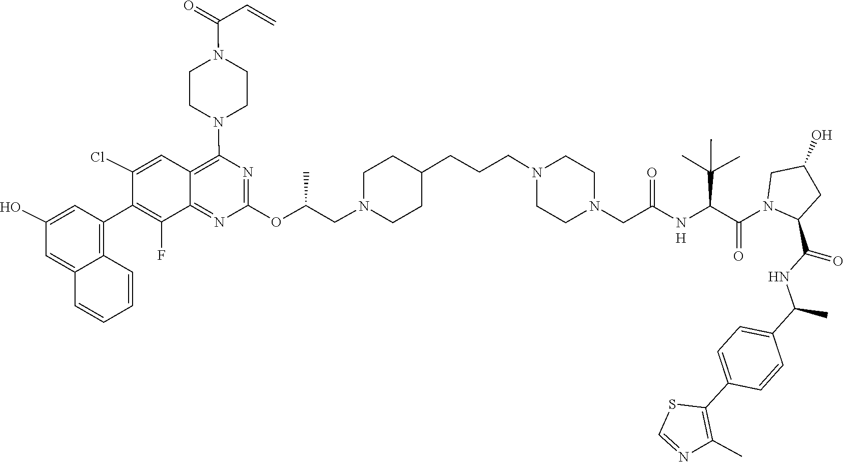

(formic acid salt, white solid). LC/MS (ESI) m/z: 1215.2 [M+H]⁺. ¹H-NMR (400 MHz, DMSO-d₆) δ 8.99 (s, 1H), 8.44 (d, J=7.6 Hz, 1H), 8.24 (s, 1H), 8.01 (s, 1H), 7.82 (d, J=8.0 Hz, 1H), 7.70 (d, J=9.6 Hz, 1H), 7.48-7.41 (m, 3H), 7.37 (d, J=8.4 Hz, 2H), 7.30 (d, J=2.4 Hz, 1H), 7.26-7.19 (m, 2H), 7.10-7.04 (m, 1H), 6.86-6.82 (m, 1H), 6.21-6.17 (m, 1H), 5.86-5.64 (m, 1H), 5.51-5.34 (m, 1H), 5.25-5.04 (m, 1H), 4.9 1-4.87 (m, 1H), 4.55-4.39 (m, 2H), 4.28 (s, 1H), 3.99-3.71 (m, 10H), 3.08-2.78 (m, 8H), 2.41-2.31 (m, 6H), 2.21 (t, J=7.2 Hz, 3H), 2.03 (s, 5H), 1.59-1.44 (m, 3H), 1.30 (s, 8H), 1.15-1.07 (m, 3H), 0.93 (s, 12H).

### 5. (2S,4R)-1-[(2S)-2-[[2-[4-[3-[4-[(2R)-2-[6-chloro-8-fluoro-7-(3-hydroxy-1-naphthyl)-4-(4-prop-2-enoylpiperazin-1-yl)quinazolin-2-yl]oxypropyl]piperazin-1-yl]propyl]-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

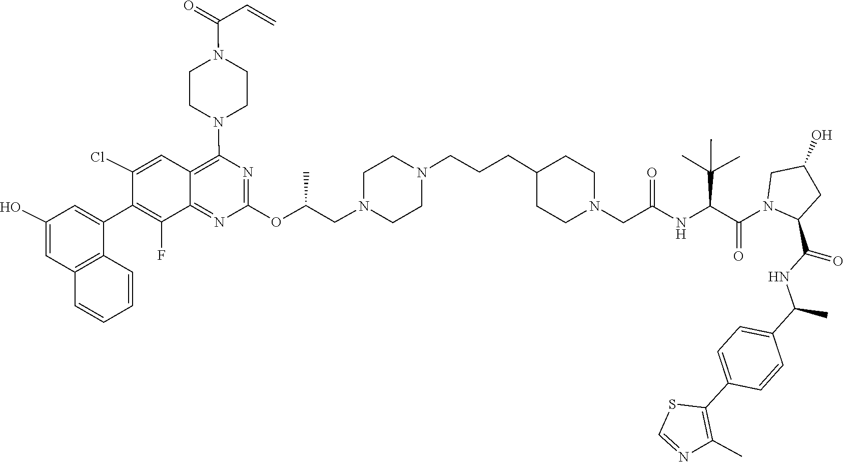 [SMILES: C=CC(=O)N1CCN(c2nc(O[C@H](C)CN3CCN(CCCC4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1]

(formic acid salt, white solid). LC/MS (ESI) m/z: 1215.6 [M+H]⁺. ¹H-NMR (400 MHz, DMSO-d₆) δ 8.98 (s, 1H), 8.44 (d, J=7.8 Hz, 1H), 8.30 (s, 1H), 8.01 (s, 1H), 7.82 (d, J=8.8 Hz, 1H), 7.73 (d, J=9.4 Hz, 1H), 7.47-7.42 (m, 3H), 7.39-7.34 (m, 2H), 7.30 (d, J=2.3 Hz, 1H), 7.25-7.18 (m, 2H), 7.10-7.04 (m, 1H), 6.84 (dd, J=10.4, 16.7 Hz, 1H), 6.19 (dd, J=2.2, 16.7 Hz, 1H), 5.85-5.64 (m, 1H), 5.49-5.35 (m, 1H), 5.20-5.07 (m, 1H), 4.89 (t, J=7.3 Hz, 1H), 4.53-4.38 (m, 2H), 4.32-4.22 (m, 1H), 4.00-3.75 (m, 9H), 3.62-3.51 (m, 4H), 2.90-2.73 (m, 5H), 2.28-1.94 (m, 13H), 1.72-1.58 (m, 3H), 1.42-1.29 (m, 9H), 1.21-1.11 (m, 5H), 0.92 (s, 9H).

### 6. (2S,4R)-1-[(2S)-2-[[2-[4-[2-[4-[(2R)-2-[6-chloro-8-fluoro-7-(3-hydroxy-1-naphthyl)-4-(4-prop-2-enoylpiperazin-1-yl)quinazolin-2-yl]oxypropyl]piperazin-1-yl]ethoxy]-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

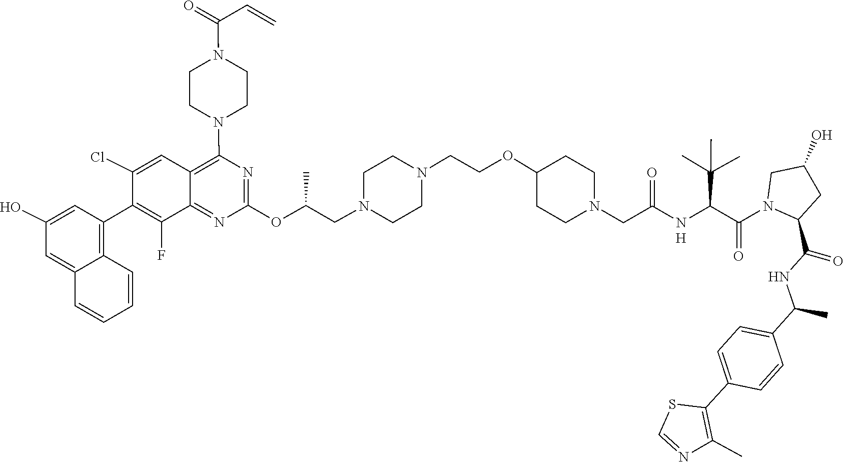

(formic acid salt, white solid). LC/MS (ESI) m/z: 1217.6 [M+H]⁺. ¹H-NMR (400 MHz, DMSO-d₆) δ 8.97 (s, 1H), 8.43 (d, J=7.7 Hz, 1H), 8.19 (s, 1H), 8.00 (s, 1H), 7.80 (d, J=8.4 Hz, 1H), 7.74 (d, J=9.5 Hz, 1H), 7.46-7.39 (m, 3H), 7.35 (d, J=8.2 Hz, 2H), 7.30-7.16 (m, 3H), 7.09-7.03 (m, 1H), 6.83 (dd, J=10.5, 16.8 Hz, 1H), 6.17 (dd, J=2.2, 16.8 Hz, 1H), 5.77-5.70 (m, 1H), 5.41 (d, J=5.6 Hz, 1H), 5.27-4.79 (m, 2H), 4.50-4.39 (m, 2H), 4.27 (s, 1H), 3.94-3.75 (m, 7H), 3.62-3.53 (m, 1H), 3.44 (d, J=6.2 Hz, 6H), 3.29-3.27 (m, 2H), 3.03-2.80 (m, 2H), 2.70-2.56 (m, 4H), 2.45 (s, 4H), 2.40-2.35 (m, 4H), 2.33-2.16 (m, 6H), 2.09-1.99 (m, 1H), 1.87-1.70 (m, 3H), 1.47-1.34 (m, 5H), 1.30 (d, J=6.2 Hz, 2H), 0.92 (s, 9H).

### 7. (2S,4R)-1-((2S)-2-(2-(4-(2-((1-((2R)-2-((4-(4-acryloylpiperazin-1-yl)-6-chloro-8- fluoro-7-(3-hydroxynaphthalen-1-yl)quinazolin-2-yl)oxy)propyl)piperidin-4-yl)oxy)ethyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

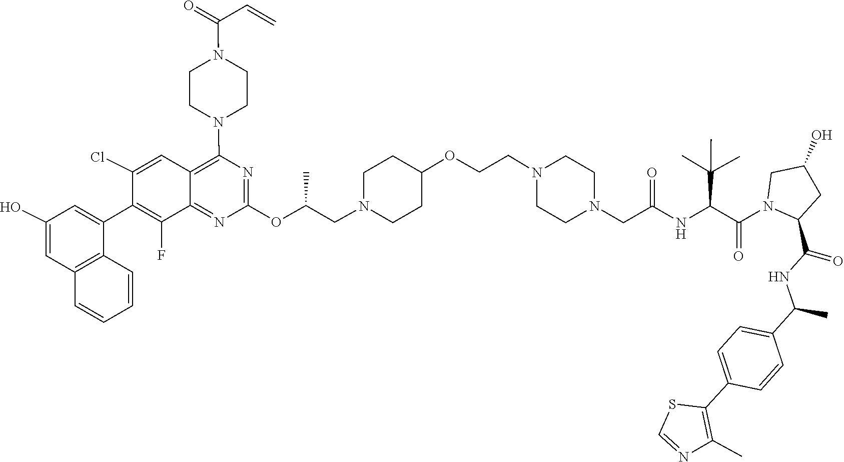 [SMILES: C=CC(=O)N1CCN(c2nc(O[C@H](C)CN3CCC(OCCN4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1]

(formic acid salt, white solid). LC/MS (ESI) m/z: 1217.6[M+H]⁺. ¹H-NMR (400 MHz, DMSO-d₆) δ 8.98 (s, 1H), 8.43 (d, J=7.6 Hz, 1H), 8.32 (s, 1H), 8.00 (s, 1H), 7.81 (d, J=8.4 Hz, 1H), 7.69 (d, J=9.6 Hz, 1H), 7.48-7.31 (m, 5H), 7.29-7.19 (m, 3H), 7.06 (dd, J=2.4, 6.1 Hz, 1H), 6.83 (dd, J=10.4, 16.7 Hz, 1H), 6.18 (dd, J=2.3, 16.7 Hz, 1H), 5.79-5.71 (m, 1H), 5.45-5.36 (m, 1H), 5.12 (s, 1H), 4.93-4.85 (m, 1H), 4.50-4.34 (m, 2H), 4.28-4.21 (m, 1H), 3.94-3.75 (m, 7H), 3.61-3.53 (m, 1H), 3.45 (s, 4H), 3.31-3.31 (m, 2H), 3.01 (d, J=16.3 Hz, 2H), 2.89-2.70 (m, 4H), 2.61 (dd, J=7.0, 12.6 Hz, 2H), 2.45-2.35 (m, 12H), 2.18-2.00 (m, 3H), 1.80-1.66 (m, 3H), 1.37 (d, J=6.9 Hz, 2H), 1.33-1.21 (m, 5H), 0.91 (s, 9H).

### 8. (2S,4R)-1-[(2S)-2-[[2-[3-[3-[4-[(2R)-2-[6-chloro-8-fluoro-7-(3-hydroxy-1-naphthyl)-4-(4-prop-2-enoylpiperazin-1-yl)quinazolin-2-yl]oxypropyl]piperazin-1-yl]propoxy]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

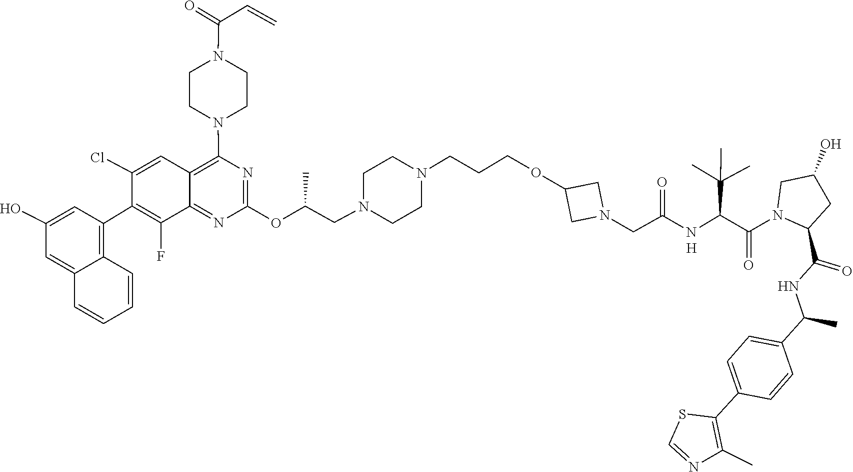

(formic acid salt, white solid). LC/MS (ESI) m/z: 1203.5 [M+H]⁺. ¹H-NMR (400 MHz, DMSO-d₆) δ 8.99 (s, 1H), 8.43 (d, J=7.6 Hz, 1H), 8.20 (s, 1H), 8.01 (s, 1H), 7.82 (d, J=8.1 Hz, 1H), 7.53 (d, J=9.6 Hz, 1H), 7.48-7.42 (m, 3H), 7.40-7.34 (m, 2H), 7.30 (d, J=2.2 Hz, 1H), 7.25-7.19 (m, 2H), 7.07 (dd, J=2.4, 4.8 Hz, 1H), 6.84 (dd, J=10.4, 16.8 Hz, 1H), 6.19 (dd, J=2.4, 16.8 Hz, 1H), 5.79-5.72 (m, 1H), 5.41 (dt, J=6.4, 12.4 Hz, 1H), 4.91 (quin, J=7.2 Hz, 1H), 4.53-4.40 (m, 2H), 4.28 (br s, 1H), 4.07 (br t, J=5.6 Hz, 1H), 3.97-3.79 (m, 9H), 3.61 (br d, J=6.8 Hz, 5H), 3.30 (br s, 4H), 3.10 (br d, J=8.8 Hz, 2H), 2.96 (br dd, J=6.8, 16.4 Hz, 3H), 2.66-2.59 (m, 2H), 2.46 (s, 3H), 2.39 (br d, J=8.4 Hz, 2H), 2.27-2.20 (m, 4H), 2.09-2.01 (m, 1H), 1.77 (ddd, J=4.6, 8.8, 13.2 Hz, 1H), 1.62-1.53 (m, 2H), 1.38 (d, J=7.2 Hz, 3H), 1.31 (dd, J=2.0, 6.4 Hz, 3H), 0.92 (s, 9H).

### 9. (2S,4R)-1-[(2S)-2-[[2-[4-[1-[(2R)-2-[6-chloro-8-fluoro-7-(3-hydroxy-1- naphthyl)-4-(4-prop-2-enoylpiperazin-1-yl)quinazolin-2-yl]oxypropyl]azetidin-3-yl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

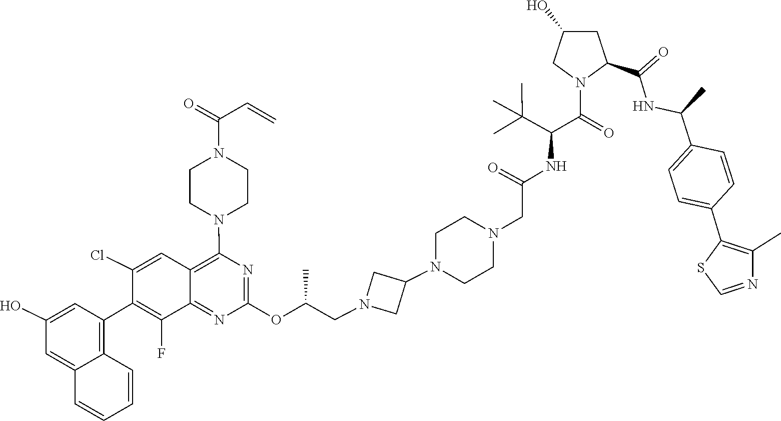 [SMILES: C=CC(=O)N1CCN(c2nc(O[C@H](C)CN3CC(N4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1]

(formic acid salt, white solid). LC/MS (ESI) m/z: 1144.3 [M+H]⁺. ¹H-NMR (400 MHz, DMSO-d₆) δ 9.90-10.17 (m, 1H), 8.98 (s, 1H), 8.42 (d, J=7.6 Hz, 1H), 8.30 (s, 1H), 7.98-8.03 (m, 1H), 7.81 (d, J=8.3 Hz, 1H), 7.62-7.70 (m, 1H), 7.41-7.45 (m, 3H), 7.34-7.38 (m, 2H), 7.28 (d, J=2.0 Hz, 1H), 7.22 (d, J=3.8 Hz, 2H), 7.07 (d, J=2.3 Hz, 1H), 6.78-6.89 (m, 1H), 6.20 (d, J=2.1 Hz, 1H), 5.75 (d, J=12.6 Hz, 1H), 5.08-5.25 (m, 2H), 4.88 (s, 1H), 4.40-4.51 (m, 2H), 4.25-4.31 (m, 1H), 3.75-3.95 (m, 10H), 3.52-3.63 (m, 4H), 2.96-3.05 (m, 2H), 2.86-2.91 (m, 2H), 2.83 (d, J=5.5 Hz, 2H), 2.69-2.74 (m, 1H), 2.56-2.60 (m, 1H), 2.45 (s, 3H), 2.14-2.27 (m, 4H), 2.00-2.08 (m, 2H), 1.71-1.79 (m, 1H), 1.34-1.39 (m, 3H), 1.26-1.30 (m, 3H), 0.91 (s, 9H).

### 10. (2S,4R)-1-[(2S)-2-[[2-[3-[4-[(2R)-2-[6-chloro-8-fluoro-7-(3-hydroxy-1- naphthyl)-4-(4-prop-2-enoylpiperazin-1-yl)quinazolin-2-yl]oxypropyl]piperazin-1-yl]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

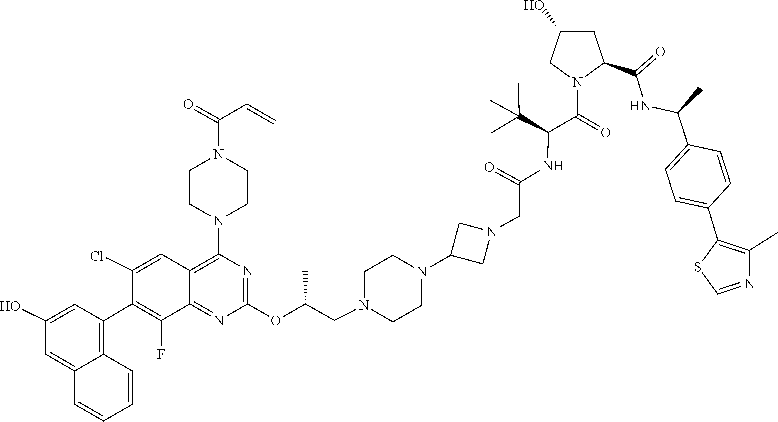 [SMILES: C=CC(=O)N1CCN(c2nc(O[C@H](C)CN3CCN(C4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1]

(formic acid salt, white solid). LC/MS (ESI) m/z: 1144.4 [M+H]⁺. ¹H-NMR (400 MHz, DMSO-d₆) δ 10.16-9.93 (m, 1H), 8.98 (s, 1H), 8.44 (d, J=7.6 Hz, 1H), 8.02 (s, 1H), 7.82 (d, J=8.4 Hz, 1H), 7.53 (d, J=9.6 Hz, 1H), 7.48-7.18 (m, 9H), 7.09-7.05 (m, 1H), 6.86-6.82 (m, 1H), 6.21-6.17 (m, 1H), 5.79-5.72 (m, 1H), 5.48-5.36 (m, 1H), 5.14 (d, J=3.2 Hz, 1H), 4.92-4.88 (m, 1H), 4.49-4.38 (m, 2H), 4.28 (s, 1H), 4.00-3.70 (m, 9H), 3.64-3.54 (m, 2H), 3.07 (s, 2H), 2.99-2.75 (m, 4H), 2.66-2.60 (m, 1H), 2.46 (s, 4H), 2.23-1.97 (m, 6H), 1.78-1.74 (m, 1H), 1.42-1.18 (m, 9H), 0.91 (s, 9H).

### 11. (2S,4R)-1-[(2S)-2-[[2-[4-[1-[(2R)-2-[6-chloro-8-fluoro-7-(3-hydroxy-1- naphthyl)-4-(4-prop-2-enoylpiperazin-1-yl)quinazolin-2-yl]oxypropyl]-4-piperidyl]-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

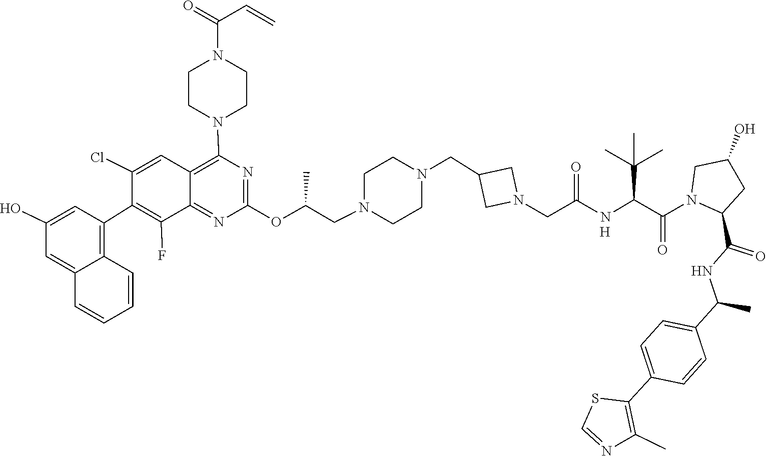

(formic acid salt, white solid). LC/MS (ESI) m/z: 1158.6 [M+H]⁺. ¹H-NMR (400 MHz, DMSO-d₆) δ 8.98 (s, 1H), 8.43 (d, J=7.2 Hz, 1H), 8.22 (s, 2H), 8.01 (s, 1H), 7.81 (d, J=8.1 Hz, 1H), 7.54-7.42 (m, 4H), 7.38-7.33 (m, 2H), 7.28 (s, 1H), 7.21 (d, J=6.4 Hz, 2H), 7.07 (d, J=4.4 Hz, 1H), 6.83 (dd, J=10.8, 16.9 Hz, 1H), 6.18 (d, J=18.4 Hz, 1H), 5.75 (d, J=12.8 Hz, 1H), 5.39 (d, J=6.0 Hz, 1H), 4.89 (br d, J=8.0 Hz, 1H), 4.54-4.38 (m, 2H), 4.27 (s, 1H), 3.92 (s, 4H), 3.85 (s, 2H), 3.78 (s, 2H), 3.55 (d, J=10.0 Hz, 2H), 3.03 (s, 4H), 2.84 (d, J=10.0 Hz, 2H), 2.72 (s, 2H), 2.45 (s, 3H), 2.41 (s, 8H), 2.24 (s, 2H), 2.04 (s, 1H), 1.75 (s, 2H), 1.37 (d, J=4.8 Hz, 3H), 1.30 (d, J=6.4 Hz, 3H), 0.91 (s, 9H).

### 12. (2S,4R)-1-[(2S)-2-[[2-[4-[[1-[(2R)-2-[6-chloro-8-fluoro-7-(3-hydroxy-1- naphthyl)-4-(4-prop-2-enoylpiperazin-1-yl)quinazolin-2-yl]oxypropyl]azetidin-3-yl]methyl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

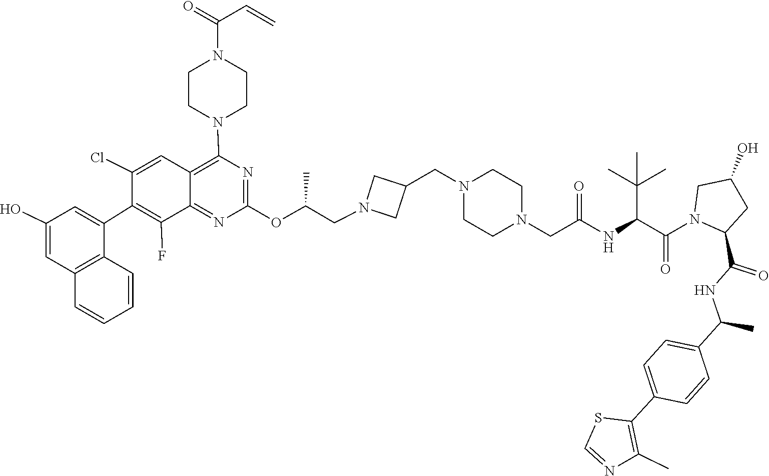 [SMILES: C=CC(=O)N1CCN(c2nc(O[C@H](C)CN3CC(CN4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1]

(formic acid salt, white solid). LC/MS (ESI) m/z: 1158.6 [M+H]⁺. ¹H-NMR (400 MHz, DMSO-d₆) δ 10.02 (br s, 1H), 8.99 (s, 1H), 8.74-8.34 (m, 1H), 8.17 (s, 1H), 8.02 (s, 1H), 7.82 (d, J=8.1 Hz, 1H), 7.68 (br d, J=9.4 Hz, 1H), 7.48-7.41 (m, 3H), 7.40-7.33 (m, 2H), 7.29 (d, J=2.3 Hz, 1H), 7.27-7.20 (m, 2H), 7.08 (d, J=2.3 Hz, 1H), 6.84 (dd, J=10.4, 16.6 Hz, 1H), 6.19 (dd, J=2.3, 16.6 Hz, 1H), 5.79-5.73 (m, 1H), 5.25-5.05 (m, 2H), 4.90 (br t, J=7.1 Hz, 1H), 4.51-4.35 (m, 2H), 4.28 (br s, 1H), 3.93 (br s, 4H), 3.86 (br s, 2H), 3.79 (br s, 3H), 3.63-3.50 (m, 3H), 3.49-3.41 (m, 4H), 3.04-2.79 (m, 5H), 2.71 (br s, 1H), 2.46 (s, 3H), 2.45-2.38 (m, 6H), 2.10-2.01 (m, 1H), 1.80-1.71 (m, 1H), 1.49-1.35 (m, 3H), 1.31-1.24 (m, 3H), 0.93 (s, 9H).

### Exemplary Synthesis of (2S,4R)-1-((S)-2-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

### Step 1: Preparation of tert-butyl (S)-2-(cyanomethyl)-4-(2-(((2S,4R)-4-(2-(2-(2-ethoxy-2-oxoethoxy)ethoxy)ethoxy)-1-methylpyrrolidin-2-yl)methoxy)-7-(naphthalen-1-yl)-5,6,7,8-tetrahydropyrido[3,4-d]pyrimidin-4-yl)piperazine-1-carboxylate

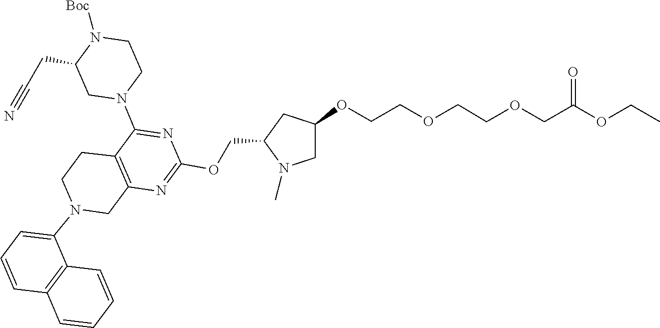 [SMILES: CCOC(=O)COCCOCCO[C@@H]1C[C@@H](COc2nc3c(c(N4CCN(C(=O)OC(C)(C)C)[C@@H](CC#N)C4)n2)CCN(c2cccc4ccccc24)C3)N(C)C1]

To a mixture of tert-butyl (2S)-2-(cyanomethyl)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (300 mg, 0.43 mmol, 1 eq) in dichloromethane (10 mL) was added rhodium(ii)acetatedimer (9 mg, 0.042 mmol, 0.1 eq), then ethyl 2-diazoacetate (146 mg, 1.28 mmol, 3 eq) was added at 0° C., then the reaction mixture was stirred at 25° C. for 12 hours. The reaction mixture was concentrated under vacuum. The residue was purified by prep-TLC (Dichloromethane:Methanol=10:1) to get compound tert-butyl (2S)-2-(cyanomethyl)-4-[2-[[(2S,4R)-4-[2-[2-(2-ethoxy-2-oxo-ethoxy)ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (111 mg, 0.14 mmol, 33% yield) as a yellow oil. LC/MS (ESI) m/z: 788.3 [M+1]⁺.

### Step 2: Preparation of 2-(2-(2-(((3R,5S)-5-(((4-((S)-4-(tert-butoxycarbonyl)-3-(cyanomethyl)piperazin-1-yl)-7-(naphthalen-1-yl)-5,6,7,8-tetrahydropyrido[3,4-cl]pyrimidin-2-yl)oxy)methyl)-1-methylpyrrolidin-3-yl)oxy)ethoxy)ethoxy)acetic Acid

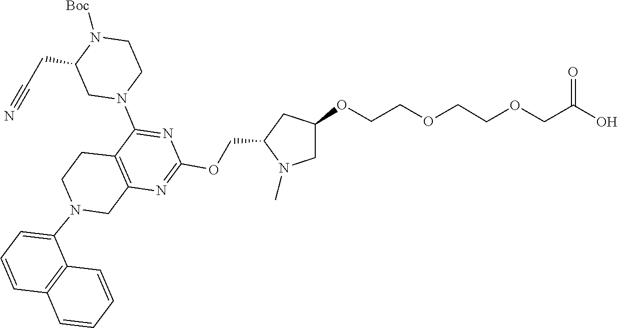 [SMILES: CN1C[C@H](OCCOCCOCC(=O)O)C[C@H]1COc1nc2c(c(N3CCN(C(=O)OC(C)(C)C)[C@@H](CC#N)C3)n1)CCN(c1cccc3ccccc13)C2]

To a mixture of tert-butyl (2S)-2-(cyanomethyl)-4-[2-[[(2S,4R)-4-[2-[2-(2-ethoxy-2-oxo-ethoxy)ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (110 mg, 0.14 mmol, 1 eq) in water (1 mL) and tetrahydrofuran (1 mL) and methanol (1 mL) was added lithiumhydroxidemonohydrate (126 mg, 3 mmol, 21.49 eq), then the reaction mixture was stirred at 25° C. for 2 hours. Tetrahydrofuran (5 mL) and waster (5 mL) was added, then the reaction mixture was adjust pH to 2-3, the aqueous phase was extracted with dichloromethane and methanol (10:1) (20 mL×4), dried with anhydrous sodium sulfate, filtered and concentrated in vacuum to get compound 2-[2-[2-[(3R,5S)-5-[[4-[(3S)-4-tert-butoxycarbonyl-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]acetic acid (131 mg) as a yellow oil.

### Step 3: Preparation of tert-butyl (S)-2-(cyanomethyl)-4-(2-(((2S,4R)-4-(2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)ethoxy)-1-methylpyrrolidin-2-yl)methoxy)-7-(naphthalen-1-yl)-5,6,7,8-tetrahydropyrido[3,4-cl]pyrimidin-4-yl)piperazine-1-carboxylate

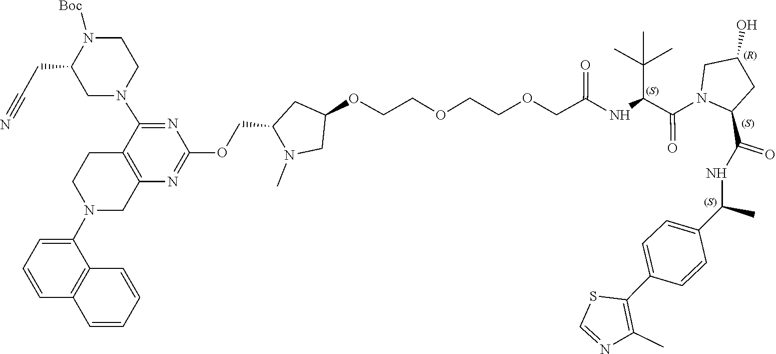

To a mixture of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (134 mg, 0.28 mmol, 2 eq, hydrochloride) and 2-[2-[2-[(3R,5S)-5-[[4-[(3S)-4-tert-butoxycarbonyl-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]acetic acid (106 mg, 0.14 mmol, 1 eq) in N-methyl-2-pyrrolidone (4 mL) was added 1-hydroxybenzotriazole (28 mg, 0.21 mmol, 1.5 eq), 1-(3-dimethylaminopropyl)-3-ethylcarbodiimide (40 mg, 0.21 mmol, 1.5 eq), N,N-diisopropylethylamine (108 mg, 0.84 mmol, 0.1 mL, 6 eq), then the reaction mixture was stirred at 20° C. for 12 hours. The residue was poured into ice-water (20 mL). The aqueous phase was extracted with ethyl acetate (20 mL*3). The combined organic phase was washed with brine (20 mL), dried with anhydrous sodium sulfate, filtered and concentrated in vacuum. The residue was purified by prep-TLC (Dichloromethane:Methanol=10:1) to get compound tert-butyl (2S)-2-(cyanomethyl)-4-[2-[[(2S,4R)-4-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (90 mg) as a yellow oil. LC/MS (ESI) m/z: 594.0 [M/2+1]⁺.

### Step 4: Preparation of (2S,4R)-1-((S)-2-(2-(2-(2-(((3R,5S)-5-(((4-((S)-3-(cyanomethyl)piperazin-1-yl)-7-(naphthalen-1-yl)-5,6,7,8-tetrahydropyrido[3,4-cl]pyrimidin-2-yl)oxy)methyl)-1-methylpyrrolidin-3-yl)oxy)ethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

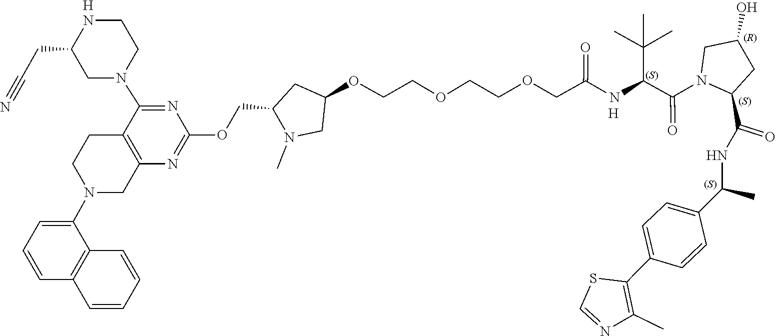

To a solution of tert-butyl (2S)-2-(cyanomethyl)-4-[2-[[(2S,4R)-4-[2-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (35 mg, 0.030 mmol, 1 eq) in dichloromethane (3 mL) was added trifluoroacetic acid (462 mg, 4.05 mmol, 0.3 mL, 137.35 eq). The mixture was stirred at 15° C. for 20 minutes. The reaction mixture was concentrated under vacuum to get compound (2S,4R)-1-[(2S)-2-[[2-[2-[2-[(3R,5S)-5-[[4-[(3S)-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (35 mg, 0.030 mmol, 99% yield, trifluoroacetate) as a yellow oil. LC/MS (ESI) m/z: 1086.7 [M+1]⁺.

### Step 5: Preparation of (2S,4R)-1-((S)-2-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-cl]pyrimidin-2-yl)oxy)methyl)-1-methylpyrrolidin-3-yl)oxy)ethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

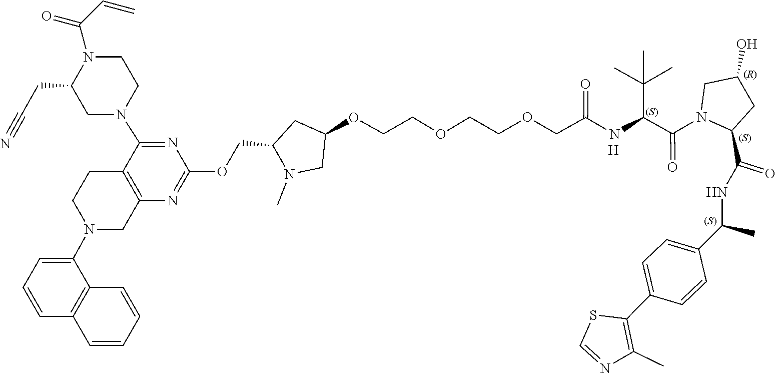

To a mixture of (2S,4R)-1-[(2S)-2-[[2-[2-[2-[(3R,5S)-5-[[4-[(3S)-3-(cyanomethyl)piperazin- 1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (30 mg, 0.025 mmol, 1 eq, trifluoroacetate) in dichloromethane (5 mL) was added 2,6-lutidine (27 mg, 0.25 mmol, 10 eq), then prop-2-enoyl chloride (2 mg, 0.022 mmol, 0.9 eq) in dichloromethane (1.8 mL) was added at −65° C., then the reaction mixture was stirred at −65° C. for 10 minutes. Water (10 mL) and stirred for 0.5 minutes. The aqueous phase was extracted with dichloromethane (20 mL×2) and concentrated in vacuum. The residue was purified by semi-preparative reverse phase HPLC, then the collected fraction was concentrated to remove most of the acetonitrile. The solution was lyophilized to get compound (2S,4R)-1-[(2S)-2-[[2-[2-[2-[(3R,5S)-5-[[4-[(3S)-3-(cyanomethyl)- 4-prop-2-enoyl-piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (9.1 mg, 0.007 mmol, 30% yield, 97% purity, formate) as a white solid. LC/MS (ESI) m/z: 570.8 [M/2+1]⁺; ¹H-NMR (400 MHz, DMSO-d₆) δ 8.98 (s, 1H), 8.44 (br d, J=7.6 Hz, 1H), 8.30 (s, 1H), 8.22-8.14 (m, 1H), 7.97-7.88 (m, 1H), 7.64 (d, J=8.2 Hz, 1H), 7.59-7.49 (m, 2H), 7.48-7.39 (m, 3H), 7.38-7.33 (m, 2H), 7.22 (d, J=7.2 Hz, 1H), 6.88 (br s, 1H), 6.20 (br d, J=17.4 Hz, 1H), 5.78 (br d, J=11.6 Hz, 1H), 5.06-4.63 (m, 2H), 4.55 (br d, J=9.5 Hz, 1H), 4.45 (br t, J=8.1 Hz, 1H), 4.34-4.22 (m, 2H), 4.17-4.09 (m, 3H), 4.08-3.98 (m, 3H), 3.98-3.91 (m, 2H), 3.61 (br d, J=3.1 Hz, 2H), 3.58 (br d, J=3.9 Hz, 4H), 3.56-3.53 (m, 1H), 3.54 (br s, 2H), 3.53-3.48 (m, 4H), 3.29 (br dd, J=6.2, 9.5 Hz, 2H), 3.24-3.17 (m, 2H), 3.07-2.90 (m, 4H), 2.78-2.63 (m, 2H), 2.45 (s, 3H), 2.34 (s, 3H), 2.19 (br dd, J=6.0, 9.4 Hz, 1H), 2.10-2.00 (m, 1H), 1.92-1.72 (m, 3H), 1.37 (br d, J=7.0 Hz, 3H), 0.94 (s, 9H).

The following compounds can be prepared in an analogous manner to synthesis of Exemplary Synthesis of (2S,4R)-1-((S)-2-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide.

### 1. (2S,4R)-1-[(2S)-2-[[2-[2-[2-[2-[2-[2-[2-[(3R,5S)-5-[[4-[(3S)-3-(cyanomethyl)-4-prop-2-enoyl-piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]ethoxy]ethoxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

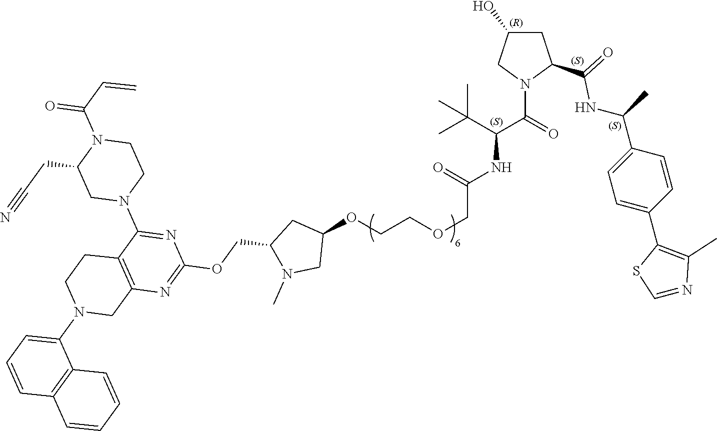

(formic acid salt, white solid). LC/MS (ESI) m/z: 1316.8 [M+H]⁺. ¹H-NMR (400 MHz, CDCl₃) δ 8.67 (s, 1H), 8.26-8.14 (m, 1H), 7.90-7.81 (m, 1H), 7.61 (d, J=8.4 Hz, 1H), 7.54-7.46 (m, 3H), 7.46-7.33 (m, 6H), 7.14 (d, J=7.2 Hz, 1H), 6.67-6.53 (m, 1H), 6.39 (d, J=15.6 Hz, 1H), 5.83 (d, J=10.8 Hz, 1H), 5.13-5.04 (m, 1H), 4.73 (t, J=8.0 Hz, 1H), 4.57 (d, J=8.8 Hz, 2H), 4.49 (s, 1H), 4.37 (dd, J=4.8, 11.6 Hz, 1H), 4.26 (s, 2H), 4.22-4.13 (m, 2H), 4.22-3.96 (m, 6H), 3.75-3.54 (m, 28H), 3.50-3.27 (m, 4H), 3.22-2.91 (m, 4H), 2.70 (s, 3H), 2.53 (s, 3H), 2.51-2.46 (m, 1H), 2.14-2.01 (m, 3H), 1.48 (d, J=6.8 Hz, 3H), 1.06 (s, 9H)

### 2. (2S,4R)-1-[(2S)-2-[[2-[2-[2-[2-[2-[2-[2-[2-[2-[(3R,5S)-5- [[4-[(3S)-3-(cyanomethyl)-4-prop-2-enoyl-piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]ethoxy]ethoxy]ethoxy]ethoxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

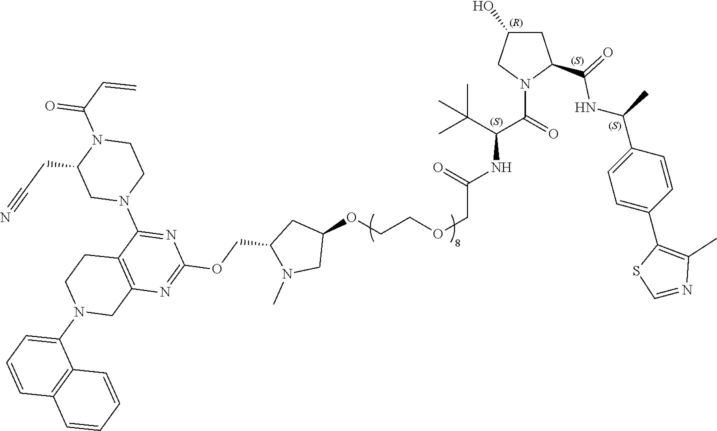

(formic acid salt, off-white solid). LC/MS (ESI) m/z: 1404.8 [M+H]⁺. ¹H-NMR (400 MHz, CDCl₃) δ 8.60 (s, 1H), 8.15-8.12 (m, 1H), 7.80-7.76 (m, 1H), 7.53 (d, J=8.0 Hz, 1H), 7.46-7.38 (m, 3H), 7.37-7.25 (m, 6H), 7.06 (d, J=7.2 Hz, 1H), 6.61-6.43 (m, 1H), 6.32 (d, J=16.4 Hz, 1H), 5.75 (d, J=10.8 Hz, 1H), 5.07-4.92 (m, 2H), 4.65 (t, J=8.0 Hz, 1H), 4.57-4.48 (m, 2H), 4.42 (brs, 1H), 4.30 (dd, J=5.2, 11.6 Hz, 1H), 4.19 (s, 2H), 4.14-4.05 (m, 2H), 4.00-3.85 (m, 5H), 3.70-3.35 (m, 40H), 3.15-3.00 (m, 1H), 2.95-2.70 (m, 4H), 2.64 (s, 3H), 2.59 (dd, J=3.2, 10.8 Hz, 1H), 2.45 (s, 3H), 2.44-2.35 (m, 1H), 2.18-2.10 (m, 1H), 2.05-1.95 (m, 2H), 1.40 (d, J=6.8 Hz, 3H), 0.99 (s, 9H).

### 3. (2S,4R)-1-[(2S)-2-[[2-[2-[2-[2-[2-[2-[2-[2-[2-[2-[2-[(3R,5S)-5-[[4-[(3S)-3-(cyanomethyl)-4-prop-2-enoyl-piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]ethoxy]ethoxy]ethoxy]ethoxy]ethoxy]ethoxy]ethoxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

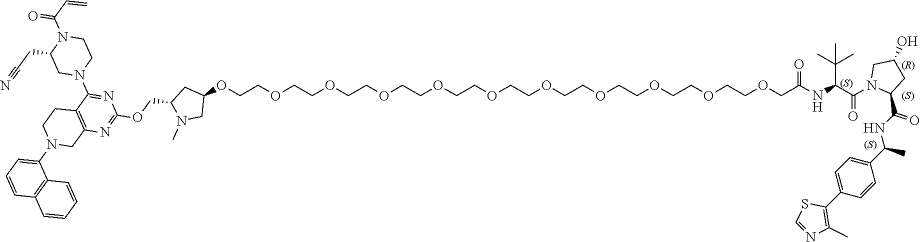

(formic acid salt, yellow solid). LC/MS (ESI) m/z: 747.1 [M/2+1]⁺. ¹H-NMR (400 MHz, CDCl₃) δ 8.85 (s, 1H), 8.24-8.09 (m, 1H), 7.91-7.80 (m, 1H), 7.62 (d, J=8.0 Hz, 1H), 7.56-7.46 (m, 3H), 7.45-7.34 (m, 6H), 7.14 (d, J=7.2 Hz, 1H), 6.67-6.51 (m, 1H), 6.39 (d, J=16.4 Hz, 1H), 5.83 (d, J=10.8 Hz, 1H), 5.11-4.86 (m, 3H), 4.81-4.67 (m, 3H), 4.57 (m, 2H), 4.37-4.29 (m, 4H), 4.27-4.21 (m, 1H), 4.11-3.98 (m, 5H), 3.73-3.57 (m, 42H), 3.53-3.47 (m, 1H), 3.43-3.30 (s, 3H), 3.22 (d, J=8.4 Hz, 1H), 3.13 (s, 3H), 3.06-2.89 (m, 3H), 2.79-2.67 (m, 1H), 2.54 (s, 3H), 2.49-2.35 (m, 2H), 2.29-2.07 (m, 2H), 1.47 (d, J=6.8 Hz, 3H), 1.05 (s, 9H).

### Exemplary Synthesis of (2S,4R)—N-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((R)-3-methyl-2-(3-methylisoxazol-5-yl)butanoyl)pyrrolidine-2-carboxamide

### Step 1: Preparation of tert-butyl (2S,4R)-2-(((tert-butyldimethylsilyl)oxy)methyl)-4-hydroxypyrrolidine-1-carboxylate

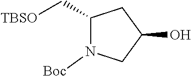 [SMILES: CC(C)(C)OC(=O)N1C[C@H](O)C[C@H]1CO[Si](C)(C)C(C)(C)C]

To a solution of tert-butyl (2S,4R)-4-hydroxy-2-(hydroxymethyl)pyrrolidine-1-carboxylate (25 g, 115.07 mmol, 1 eq) in dichloromethane (400 mL) was added triethylamine (23.29 g, 230.14 mmol, 32.03 mL, 2 eq) and N,N-dimethylpyridin-4-amine (1.41 g, 11.51 mmol, 0.1 eq), and then tert-butyldimethylsilyl chloride (18.21 g, 120.82 mmol, 1.05 eq) was added at 0° C., the mixture was stirred at 25° C. for 48 hours. Evaporate the solution on a water bath under reduced pressure using a rotary evaporator. This crude product was purified by silica gel column chromatography (EA:PE=0:1 to 1:5) to give compound tert-butyl (2S,4R)-2-[[tert-butyl(dimethyl)silyl]oxymethyl]-4-hydroxy-pyrrolidine-1-carboxylate (40 g) as a colorless oil. ¹H-NMR (400 MHz, CD₃OD) δ 4.45 (s, 1H), 4.00-3.91 (m, 1H), 3.56-3.53 (m, 2H), 3.40 (s, 2H), 2.23-2.15 (m, 2H), 2.00-1.90 (m, 1H), 1.45 (s, 9H), 0.89 (s, 9H), 0.01 (s, 6H).

### Step 2: Preparation of tert-butyl (2S,4R)-2-(((tert-butyldimethylsilyl)oxy)methyl)-4-(2-(2-((tetrahydro-2H-pyran-2-yl)oxy)ethoxy)ethoxy)pyrrolidine-1-carboxylate

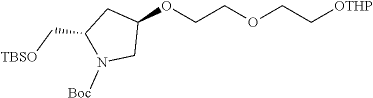

To a solution of tert-butyl (2S,4R)-2-[[tert-butyl(dimethyl)silyl]oxymethyl]-4-hydroxy-pyrrolidine-1-carboxylate (40 g, 120.66 mmol, 1 eq) in tetrahydrofuran (500 mL) was added sodium hydride (7.24 g, 180.98 mmol, 60% in mineral oil, 1.5 eq) at 0° C. The reaction mixture was stirred at 25° C. for 1 hour. Then 2-(2-tetrahydropyran-2-yloxyethoxy)ethyl 4-methylbenzenesulfonate (45.71 g, 132.72 mmol, 1.1 eq) was added and the reaction mixture was stirred at 25° C. for another 12 hours. The reaction mixture was quenched by saturated aqueous ammonium chloride (200 mL), then extracted by ethyl acetate (100 mL×3). The organic layers were combined and evaporated under vacuum to get the residue. The residue was purified through silica gel column chromatography (Petroleum ether/Ethyl acetate=1/0 to 2/1) to get the product. Tert-butyl (2S,4R)-2-[[tert-butyl(dimethyl)silyl]oxymethyl]-4-[2-(2-tetrahydropyran-2-yloxyethoxy)ethoxy]pyrrolidine-1-carboxylate (40.8 g, 83.79 mmol, 68% yield) was obtained as a light yellow oil. ¹H-NMR (400 MHz, CDCl₃) δ 4.65-4.64 (m, 1H), 3.90-3.85 (m, 4H), 3.70-3.39 (m, 12H), 2.23-2.13 (m, 1H), 2.10-1.92 (m, 1H), 1.91-1.49 (m, 6H), 1.45 (s, 9H), 0.88 (s, 9H), 0.03 (s, 6H).

### Step 3: Preparation of tert-butyl (2S,4R)-2-(hydroxymethyl)-4-(2-(2-((tetrahydro-2H-pyran-2-yl)oxy)ethoxy)ethoxy)pyrrolidine-1-carboxylate

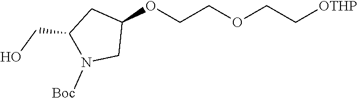

To a solution of tert-butyl (2S,4R)-2-[[tert-butyl(dimethyl)silyl]oxymethyl]-4-[2-(2-tetrahydropyran-2-yloxyethoxy)ethoxy]pyrrolidine-1-carboxylate (10.8 g, 21.44 mmol, 1 eq) in tetrahydrofuran (125 mL) was added tetrabutylammonium fluoride (1 M, 23.6 mL, 1.1 eq) at 25° C. The mixture was stirred at 25° C. for 12 hours. The solvent was removed under vacuum to get the residue. The residue was purified through silica gel column chromatography (Petroleum ether/Ethyl acetate=10/1 to 1/1). The product tert-butyl (2S,4R)-2-(hydroxymethyl)-4-[2-(2-tetrahydropyran-2-yloxyethoxy)ethoxy]pyrrolidine-1-carboxylate (6.35 g, 16.30 mmol, 76% yield) was obtained as a light yellow oil.

### Step 4: Preparation of ((2S,4R)-1-methyl-4-(2-(2-((tetrahydro-2H-pyran-2-yl)oxy)ethoxy)ethoxy)pyrrolidin-2-yl)methanol

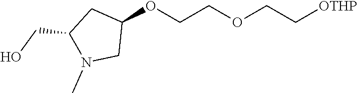

To a solution of tert-butyl (2S,4R)-2-(hydroxymethyl)-4-[2-(2-tetrahydropyran-2-yloxyethoxy)ethoxy]pyrrolidine-1-carboxylate (20 g, 51.35 mmol, 1 eq) in tetrahydrofuran (350 mL) was added lithium aluminum hydride (4.87 g, 128.38 mmol, 2.5 eq) at 25° C. The mixture was stirred at 60° C. for 16 hours. The reaction mixture was quenched by water (10 mL) before celite (20 g) was added. The mixture was filtered and the filtrate was collected, then evaporated under vacuum to get [(2S,4R)-1-methyl-4-[2-(2-tetrahydropyran-2-yloxyethoxy)ethoxy]pyrrolidin-2-yl]methanol (14 g) as a light yellow oil. ¹H-NMR (400 MHz, CDCl₃) δ 4.64-4.63 (m, 1H), 4.01-3.97 (m, 1H), 3.87-3.85 (m, 2H), 3.70-3.46 (m, 9H), 3.40-3.38 (m, 2H), 2.63-2.61 (m, 1H), 2.40-2.36 (m, 1H), 2.33 (s, 3H), 2.13-2.02 (m, 1H), 1.88-1.43 (m, 8H).

### Step 5: Preparation of tert-butyl 4-((S)-4-((benzyloxy)carbonyl)-3-(cyanomethyl)piperazin-1-yl)-2-(((2S,4R)-1-methyl-4-(2-(2-((tetrahydro-2H-pyran-2-yl)oxy)ethoxy)ethoxy)pyrrolidin-2-yl)methoxy)-5,8-dihydropyrido[3,4-d]pyrimidine-7(6H)-carboxylate

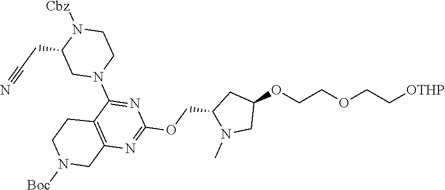

To a solution of [(2S,4R)-1-methyl-4-[2-(2-tetrahydropyran-2-yloxyethoxy)ethoxy]pyrrolidin-2-yl]methanol (6.22 g, 20.49 mmol, 1.2 eq) and tert-butyl 4-[(3S)-4-benzyloxycarbonyl-3-(cyanomethyl)piperazin-1-yl]-2-chloro-6,8-dihydro-5H-pyrido[3,4-d]pyrimidine-7-carboxylate (9 g, 17.08 mmol, 1 eq) in dioxane (180 mL) was added methanesulfonato(2-dicyclohexyl-phosphino-2′,6′-di-i-propoxy-1,1′-biphenyl)(2′-amino-1,1′-biphenyl-2-yl)palladium(II) (1.43 g, 1.71 mmol, 0.1 eq) and cesium carbonate (16.69 g, 51.23 mmol, 3 eq) under nitrogen. The reaction mixture was stirred at 90° C. for 6 hours under nitrogen. The solvent was removed under vacuum to get the crude product. The crude product was purified by silica gel column chromatography (Ethyl acetate) to get the product. The product tert-butyl 4-[(3S)-4-benzyloxycarbonyl-3-(cyanomethyl)piperazin-1-yl]-2-[[(2S,4R)-1-methyl-4-[2-(2-tetrahydropyran-2-yloxyethoxy)ethoxy]pyrrolidin-2-yl]methoxy]-6,8-dihydro-5H-pyrido[3,4-d]pyrimidine-7-carboxylate (7.4 g, 6.63 mmol, 39% yield, 71% purity) was obtained as a brown solid. LC/MS (ESI) m/z: 794.5 [M+1]⁺.

### Step 6: Preparation of benzyl (S)-2-(cyanomethyl)-4-(2-(((2S,4R)-4-(2-(2-hydroxyethoxy)ethoxy)-1-methylpyrrolidin-2-yl)methoxy)-5,6,7,8-tetrahydropyrido[3,4-d]pyrimidin-4-yl)piperazine-1-carboxylate

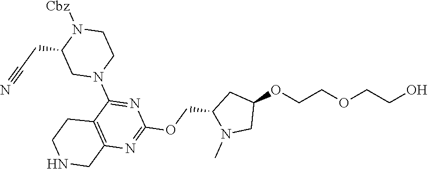 [SMILES: CN1C[C@H](OCCOCCO)C[C@H]1COc1nc2c(c(N3CCN(C(=O)OCc4ccccc4)[C@@H](CC#N)C3)n1)CCNC2]

To a solution of tert-butyl 4-[(3S)-4-benzyloxycarbonyl-3-(cyanomethyl)piperazin-1-yl]-2-[[(2S,4R)-1-methyl-4-[2-(2-tetrahydropyran-2-yloxyethoxy)ethoxy]pyrrolidin-2-yl]methoxy]-6,8-dihydro-5H-pyrido[3,4-d]pyrimidine-7-carboxylate (7 g, 8.82 mmol, 1 eq) in dichloromethane (100 mL) was added trifluoroacetic acid (25.13 g, 220.42 mmol, 16.3 mL, 25 eq). The mixture was stirred at 25° C. for 2 hours. The reaction mixture was quenched by saturated aqueous sodium bicarbonate solution (100 mL), then extracted by dichloromethane (30 mL×3). The combined organic layers were evaporated under vacuum to remove the solvent to get the crude product. The crude product was purified by Prep-HPLC. The product benzyl (2S)-2-(cyanomethyl)-4-[2-[[(2S,4R)-4-[2-(2-hydroxyethoxy)ethoxy]-1-methyl-pyrrolidin-2-yl]methoxy]-5,6,7,8-tetrahydropyrido[3,4-d]pyrimidin-4-yl]piperazine-1-carboxylate (2.76 g, 4.13 mmol, 47% yield, 91% purity) was obtained as a light yellow solid. LC/MS (ESI) m/z: 610.4 [M+1]⁺; ¹H-NMR (400 MHz, CDCl₃) δ 7.40-7.37 (m, 5H), 5.19 (s, 2H), 4.65 (s, 1H), 4.37 (dd, J=11.2 Hz, J=4.4 Hz, 1H), 4.18-2.58 (m, 26H), 2.46 (s, 3H), 2.38 (dd, J=9.6 Hz, J=6.0 Hz, 1H), 2.01-1.95 (m, 3H).

### Step 7: Preparation of benzyl (S)-2-(cyanomethyl)-4-(2-(((2S,4R)-4-(2-(2-hydroxyethoxy)ethoxy)-1-methylpyrrolidin-2-yl)methoxy)-7-(naphthalen-1-yl)-5,6,7,8-tetrahydropyrido[3,4-d]pyrimidin-4-yl)piperazine-1-carboxylate

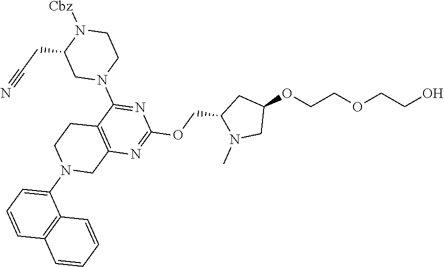 [SMILES: CN1C[C@H](OCCOCCO)C[C@H]1COc1nc2c(c(N3CCN(C(=O)OCc4ccccc4)[C@@H](CC#N)C3)n1)CCN(c1cccc3ccccc13)C2]

To a solution of benzyl (2S)-2-(cyanomethyl)-4-[2-[[(2S,4R)-4-[2-(2-hydroxyethoxy)ethoxy]-1-methyl-pyrrolidin-2-yl]methoxy]-5,6,7,8-tetrahydropyrido[3,4-d]pyrimidin-4-yl]piperazine-1-carboxylate (2.2 g, 3.61 mmol, 1 eq) and 1-bromonaphthalene (1.34 g, 6.49 mmol, 0.9 mL, 1.8 eq) in dioxane (50 mL) was added methanesulfonato(2-dicyclohexylphosphino-2′,6′-di-i-propoxy-1,1′-biphenyl)(2′-amino-1,1′-biphenyl-2-yl)palladium(II) (301.8 mg, 0.36 mmol, 0.1 eq) and cesium carbonate (3.53 g, 10.82 mmol, 3 eq) in nitrogen. The mixture was stirred at 90° C. for 12 hours in nitrogen. The reaction mixture was quenched by water (50 mL) and extracted by ethyl acetate (40 mL×3). The organic layers were combined and evaporated under vacuum to get the residue. The residue was purified through silica gel column chromatography (Dichloromethane/Methanol=10/1) to get the product. The product benzyl (2S)-2-(cyanomethyl)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (1.15 g, 1.39 mmol, 38% yield, 89% purity) was obtained as a yellow oil. LC/MS (ESI) m/z: 736.3 [M+1]⁺; ¹H-NMR (400 MHz, CDCl₃) δ 8.22-8.20 (m, 1H), 7.87-7.85 (m, 1H), 7.62 (d, J=8.0 Hz, 1H), 7.51-7.36 (m, 8H), 7.15 (d, J=6.8 Hz, 1H), 5.21 (s, 2H), 4.70 (s, 1H), 4.38-4.10 (m, 8H), 3.76-3.31 (m, 14H), 3.10-2.72 (m, 6H), 2.47 (s, 3H), 2.39-2.35 (m, 1H), 2.14-2.08 (m, 1H), 2.03-1.96 (m, 1H).

### Step 8: Preparation of 2-((S)-4-(2-(((2S,4R)-4-(2-(2-hydroxyethoxy)ethoxy)-1-methylpyrrolidin-2-yl)methoxy)-7-(naphthalen-1-yl)-5,6,7,8-tetrahydropyrido[3,4-cl]pyrimidin-4-yl)piperazin-2-yl)acetonitrile

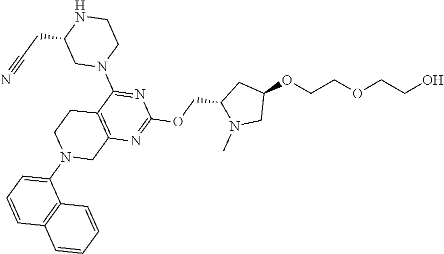 [SMILES: CN1C[C@H](OCCOCCO)C[C@H]1COc1nc2c(c(N3CCN[C@@H](CC#N)C3)n1)CCN(c1cccc3ccccc13)C2]

To a solution of benzyl (2S)-2-(cyanomethyl)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (1.38 g, 1.88 mmol, 1 eq) and ammonium hydroxide (1.82 g, 12.98 mmol, 2 mL, 25% purity, 6.92 eq) in methanol (60 mL) and tetrahydrofuran (3 mL) was added palladium on activated carbon catalyst (200 mg, 10% purity). The mixture was degassed and charged with hydrogen, then stirred at 25° C. with hydrogen (15 psi) for 12 hours. The reaction mixture was added tetrahydrofuran (40 mL) and filtered. The organic solvent was removed under vacuum to get 2-[(2S)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazin-2-yl]acetonitrile (1.1 g) as a light yellow solid. LC/MS (ESI) m/z: 602.3 [M+1]⁺; ¹H-NMR (400 MHz, CDCl₃) δ 8.23-8.21 (m, 1H), 7.87-7.85 (m, 1H), 7.62 (d, J=8.4 Hz, 1H), 7.51-7.49 (m, 2H), 7.43 (t, J=8.4 Hz, 1H), 7.15-7.13 (m, 1H), 4.41-4.39 (m, 1H), 4.26 (s, 2H), 4.22 (dd, J=11.2 Hz, J=6.4 Hz, 1H), 4.03 (d, J=12.0 Hz, 1H), 3.88 (d, J=12.0 Hz, 1H), 3.76-3.72 (m, 3H), 3.67-3.54 (m, 6H), 3.45-2.85 (m, 12H), 2.57-2.55 (m, 2H), 2.47 (s, 3H), 2.39 (dd, J=9.6 Hz, J=6.0 Hz, 1H), 2.11-1.84 (m, 2H).

### Step 9: Preparation of tert-butyl (S)-2-(cyanomethyl)-4-(2-(((2S,4R)-4-(2-(2-hydroxyethoxy)ethoxy)-1-methylpyrrolidin-2-yl)methoxy)-7-(naphthalen-1-yl)-5,6,7,8-tetrahydropyrido[3,4-d]pyrimidin-4-yl)piperazine-1-carboxylate

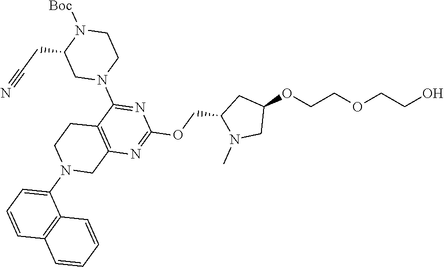 [SMILES: CN1C[C@H](OCCOCCO)C[C@H]1COc1nc2c(c(N3CCN(C(=O)OC(C)(C)C)[C@@H](CC#N)C3)n1)CCN(c1cccc3ccccc13)C2]

To a solution of 2-[(2S)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazin-2-yl]acetonitrile (800 mg, 1.33 mmol, 1 eq) in tetrahydrofuran (30 mL) and water (30 mL) was added sodium bicarbonate (223.4 mg, 2.66 mmol, 0.1 mL, 2 eq) and di-tert-butyl dicarbonate (1.45 g, 6.65 mmol, 1.5 mL, 5 eq). The mixture was stirred at 25° C. for 6 hours. The reaction mixture was quenched by adding water (20 mL), then extracted by ethyl acetate (30 mL×3). The organic phase was washed with brine (30 mL×2), evaporated under vacuum to get the crude product. The crude product was purified through silica gel column chromatography (Dichloromethane/Methanol=10/1). The product tert-butyl (2S)-2-(cyano-methyl)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (670 mg, 0.95 mmol, 72% yield) was obtained as a light yellow solid. LC/MS (ESI) m/z: 702.4 [M+1]⁺; ¹H-NMR (400 MHz, CDCl₃) δ 8.23-8.20 (m, 1H), 7.87-7.85 (m, 1H), 7.62 (d, J=8.4 Hz, 1H), 7.51-7.49 (m, 2H), 7.43 (t, J=8.4 Hz, 1H), 7.15-7.14 (m, 1H), 4.63 (s, 1H), 4.41-4.39 (m, 1H), 4.26-4.22 (m, 3H), 4.12 (d, J=12.0 Hz, 1H), 3.75-3.31 (m, 14H), 3.10-2.76 (m, 6H), 2.48 (s, 3H), 2.40 (dd, J=9.6 Hz, J=6.0 Hz, 1H), 2.37-2.05 (m, 2H), 1.52 (s, 9H).

### Step 10: Preparation of tert-butyl (S)-2-(cyanomethyl)-4-(2-(((2S,4R)-1-methyl-4-(2-(2-(tosyloxy)ethoxy)ethoxy)pyrrolidin-2-yl)methoxy)-7-(naphthalen-1-yl)-5,6,7,8-tetrahydropyrido[3,4-d]pyrimidin-4-yl)piperazine-1-carboxylate

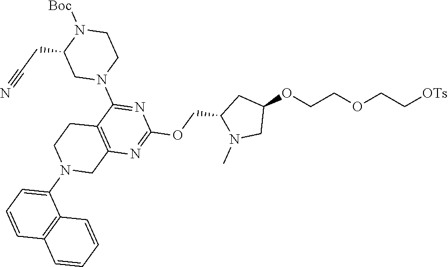 [SMILES: Cc1ccc(S(=O)(=O)OCCOCCO[C@@H]2C[C@@H](COc3nc4c(c(N5CCN(C(=O)OC(C)(C)C)[C@@H](CC#N)C5)n3)CCN(c3cccc5ccccc35)C4)N(C)C2)cc1]

To a solution of tert-butyl (2S)-2-(cyanomethyl)-4-[2-[[(2S,4R)-4-[2-(2-hydroxyethoxy)ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (300 mg, 0.43 mmol, 1 eq) in dichloromethane (5 mL) was added triethylamine (129.8 mg, 1.28 mmol, 0.2 mL, 3 eq), p-toluenesulfonyl chloride (163 mg, 0.85 mmol, 2 eq) and dimethylaminopyridine (10.4 mg, 0.09 mmol, 0.2 eq). The mixture was stirred at 25° C. for 12 hours. The solvent was removed under vacuum to get a residue. The residue was purified by prep-TLC (silicon dioxide, Dichloromethane/Methanol=10/1). The product tert-butyl (2S)-2-(cyanomethyl)-4-[2-[[(2S,4R)-1-methyl-4-[2-[2-(p-tolylsulfonyloxy)ethoxy]ethoxy]pyrrolidin-2-yl]methoxy]-7-(1-naphthyl)-6,8-dihydro-5H-pyrido[3,4-d]pyrimidin-4-yl]piperazine-1-carboxylate (233 mg, 0.27 mmol, 64% yield) was obtained as a light yellow solid. ¹H-NMR (400 MHz, CDCl₃) δ 8.22-8.20 (m, 1H), 7.87-7.85 (m, 1H), 7.81-7.75 (m, 2H), 7.62 (d, J=8.0 Hz, 1H), 7.51-7.49 (m, 2H), 7.43 (t, J=8.0 Hz, 1H), 7.35 (d, J=8.0 Hz, 2H), 7.15-7.12 (m, 1H), 4.63 (s, 1H), 4.43-4.39 (m, 1H), 4.27-3.93 (m, 8H), 3.72-3.27 (m, 12H), 3.07-2.75 (m, 6H), 2.49 (s, 3H), 2.45 (s, 3H), 2.41-2.36 (m, 1H), 2.09-1.98 (m, 2H), 1.52 (s, 9H).

### Step 11: Preparation of tert-butyl (S)-2-(cyanomethyl)-4-(2-(((2S,4R)-4-(2-(2-(2-(((2S,4R)-4-hydroxy-1-((R)-3-methyl-2-(3-methylisoxazol-5-yl)butanoyl)pyrrolidine-2-carboxamido)methyl)-5-(4-methylthiazol-5-yl)phenoxy)ethoxy)ethoxy)-1-methylpyrrolidin-2-yl)methoxy)-7-(naphthalen-1-yl)-5,6,7,8-tetrahydropyrido[3,4-d]pyrimidin-4-yl)piperazine-1-carboxylate and tert-butyl (S)-2-(cyanomethyl)-4-(2-(((2S,4R)-4-(2-(2-(2-(((2S,4R)-4-hydroxy-1-((S)-3-methyl-2-(3-methylisoxazol-5-yl)butanoyl)pyrrolidine-2-carboxamido)methyl)-5-(4-methylthiazol-5-yl)phenoxy)ethoxy)ethoxy)-1-methylpyrrolidin-2-yl)methoxy)-7-(naphthalen-1-yl)-5,6,7,8-tetrahydropyrido[3,4-d]pyrimidin-4-yl)piperazine-1-carboxylate

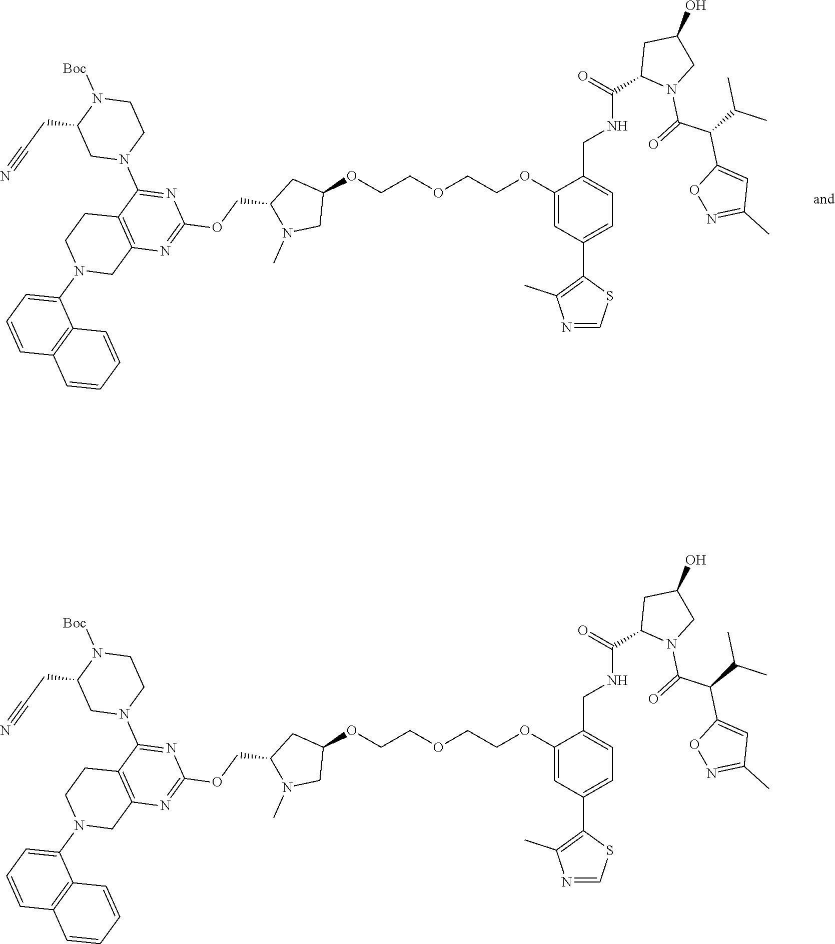 [SMILES: Cc1cc([C@H](C(=O)N2C[C@H](O)C[C@H]2C(=O)NCc2ccc(-c3scnc3C)cc2OCCOCCO[C@@H]2C[C@@H](COc3nc4c(c(N5CCN(C(=O)OC(C)(C)C)[C@@H](CC#N)C5)n3)CCN(c3cccc5ccccc35)C4)N(C)C2)C(C)C)on1 ; Cc1cc([C@@H](C(=O)N2C[C@H](O)C[C@H]2C(=O)NCc2ccc(-c3scnc3C)cc2OCCOCCO[C@@H]2C[C@@H](COc3nc4c(c(N5CCN(C(=O)OC(C)(C)C)[C@@H](CC#N)C5)n3)CCN(c3cccc5ccccc35)C4)N(C)C2)C(C)C)on1]

To a solution of tert-butyl (2S)-2-(cyanomethyl)-4-[2-[[(2S,4R)-1-methyl-4-[2-[2-(p-tolyl-sulfonyloxy)ethoxy]ethoxy]pyrrolidin-2-yl]methoxy]-7-(1-naphthyl)-6,8-dihydro-5H-pyrido[3,4-d]pyrimidin-4-yl]piperazine-1-carboxylate (250 mg, 0.29 mmol, 1 eq) and (2S,4R)-4-hydroxy-N-[[2-hydroxy-4-(4-methylthiazol-5-yl)phenyl]methyl]-1-[3-methyl-2-(3-methylisoxazol-5-yl)butanoyl]pyrrolidine-2-carboxamide (152.9 mg, 0.31 mmol, 1.05 eq) in acetonitrile (8 mL) was added cesium carbonate (190.3 mg, 0.58 mmol, 2 eq). The mixture was stirred at 80° C. for 14 hours. The mixture was extracted by ethyl acetate (30 mL×3) after water (30 mL) was added. The combined organic phases were evaporated under vacuum to get a residue. The residue was purified by Prep-TLC (silicon dioxide, Dichloromethane/Methanol=10/1). The product containing the two isomers (255 mg) was obtained as a yellow solid. Then the product was further purified through SFC. The product tert-butyl (2S)-2-(cyanomethyl)-4-[2-[[(2S,4R)-4-[2-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (80 mg, 0.06 mmol, 21% yield, 92% purity) was obtained as a yellow oil. The product tert-butyl (2S)-2-(cyanomethyl)-4-[2-[[(2S,4R)-4-[2-[2-[2-[[[(2S,4R)-4-hydroxy-1-[(2S)-3-methyl-2-(3-methylisoxazol-5-yl)butanoyl]pyrrolidine-2-carbonyl]amino]methyl]-5-(4-methylthiazol-5-yl)phenoxy]ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (120 mg, 0.096 mmol, 33% yield, 95% purity) was obtained as a light yellow solid. LC/MS (ESI) m/z: 1182.7 [M+1]⁺.

### Step 12: Preparation of (2S,4R)—N-(2-(2-(2-(((3R,5S)-5-(((4-((S)-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((R)-3-methyl-2-(3-methylisoxazol-5-yl)butanoyl)pyrrolidine-2-carboxamide

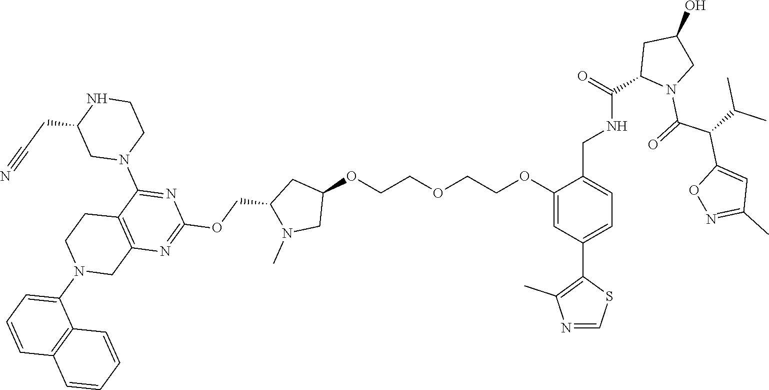 [SMILES: Cc1cc([C@H](C(=O)N2C[C@H](O)C[C@H]2C(=O)NCc2ccc(-c3scnc3C)cc2OCCOCCO[C@@H]2C[C@@H](COc3nc4c(c(N5CCN[C@@H](CC#N)C5)n3)CCN(c3cccc5ccccc35)C4)N(C)C2)C(C)C)on1]

To a solution of tert-butyl (2S)-2-(cyanomethyl)-4-[2-[[(2S,4R)-4-[2-[2-[2-[[[(2S,4R)-4-hydroxy-1-[(2R)-3-methyl-2-(3-methylisoxazol-5-yl)butanoyl]pyrrolidine-2-carbonyl]amino]methyl]-5-(4-methylthiazol-5-yl)phenoxy]ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (80 mg, 0.067 mmol, 1 eq) in dichloromethane (8 mL) was added trifluoroacetic acid (3.08 g, 27.0 mmol, 2.0 mL, 400 eq). The mixture was stirred at 20° C. for 2 hours. The reaction mixture was evaporated under vacuum to get the product, (2S,4R)—N-[[2-[2-[2-[(3R,5S)-5-[[4-[(3S)-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide (73 mg) as a yellow oil.

### Step 13: Preparation of (2S,4R)—N-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((R)-3-methyl-2-(3-methylisoxazol-5-yl)butanoyl)pyrrolidine-2-carboxamide

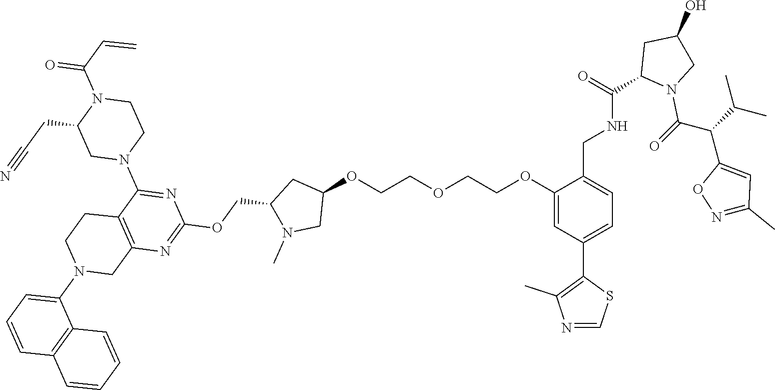 [SMILES: C=CC(=O)N1CCN(c2nc(OC[C@@H]3C[C@@H](OCCOCCOc4cc(-c5scnc5C)ccc4CNC(=O)[C@@H]4C[C@@H](O)CN4C(=O)[C@@H](c4cc(C)no4)C(C)C)CN3C)nc3c2CCN(c2cccc4ccccc24)C3)C[C@@H]1CC#N]

To a solution of (2S,4R)—N-[[2-[2-[2-[(3R,5S)-5-[[4-[(3S)-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide (73 mg, 0.067 mmol, 1 eq) and 2,6-Lutidine (230 mg, 2.15 mmol, 32 eq) in the mixed solvent of dichloromethane (8 mL) and N,N-dimethylformamide (1 mL) was added prop-2-enoyl chloride (6.1 mg, 0.067 mmol, 1 eq) in dichloromethane (0.2 mL) in nitrogen. The mixture was stirred at −65° C. for 10 minutes. The reaction mixture was quenched by water (10 mL) before warmed to 25° C., then extracted by dichloromethane (20 mL×3). The combined organic layers were combined and evaporated under vacuum to get a residue. The residue was purified through Prep-HPLC. The product (2S,4R)—N-[[2-[2-[2-[(3R,5S)-5-[[4-[(3S)-3-(cyanomethyl)-4-prop-2-enoyl-piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide (44.1 mg, 0.034 mmol, 50% yield, 96% purity, trifluoroacetate) was obtained as a light yellow solid. LC/MS (ESI) m/z: 1136.3 [M+1]⁺; ¹H-NMR (400 MHz, DMSO-d₆) δ 8.98 (s, 1H), 8.33-8.30 (m, 1H), 8.19-8.17 (m, 1H), 7.95-7.92 (m, 1H), 7.65 (d, J=8.0 Hz, 1H), 7.55-7.53 (m, 2H), 7.47 (t, J=7.6 Hz, 1H), 7.32-7.21 (m, 2H), 7.08-6.97 (m, 2H), 6.87 (s, 1H), 6.22-6.18 (m, 2H), 5.81-5.78 (m, 1H), 5.00-4.76 (m, 1H), 4.62-4.59 (m, 1H), 4.48-4.41 (m, 3H), 4.33-4.08 (m, 12H), 3.98-3.88 (m, 1H), 3.85 (d, J=8.4 Hz, 1H), 3.83-3.71 (m, 3H), 3.65-3.59 (m, 6H), 3.55-3.44 (m, 1H), 3.41-3.33 (m, 1H), 3.31-3.14 (m, 4H), 3.08-3.29 (m, 7H), 2.46-2.44 (m, 3H), 2.37-2.23 (m, 2H), 2.21-2.13 (m, 3H), 2.08-1.86 (m, 2H), 1.00-0.55 (m, 6H).

### Exemplary Synthesis of (2S,4R)—N-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((S)-3-methyl-2-(3-methylisoxazol-5-yl)butanoyl)pyrrolidine-2-carboxamide

### Step 1: Preparation of (2S,4R)—N-(2-(2-(2-(((3R,5S)-5-(((4-((S)-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((S)-3-methyl-2-(3-methylisoxazol-5-yl)butanoyl)pyrrolidine-2-carboxamide

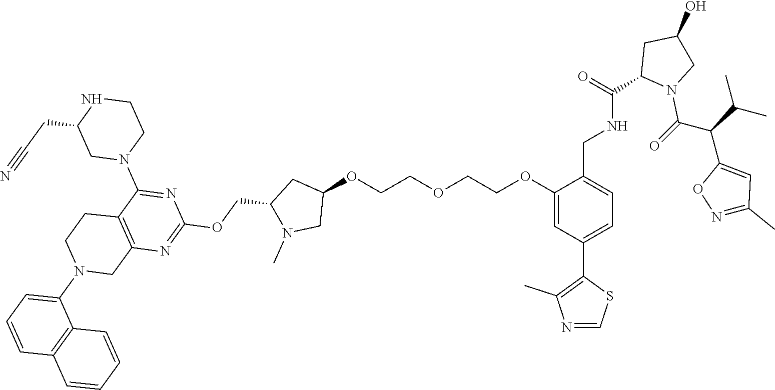 [SMILES: Cc1cc([C@@H](C(=O)N2C[C@H](O)C[C@H]2C(=O)NCc2ccc(-c3scnc3C)cc2OCCOCCO[C@@H]2C[C@@H](COc3nc4c(c(N5CCN[C@@H](CC#N)C5)n3)CCN(c3cccc5ccccc35)C4)N(C)C2)C(C)C)on1]

To a solution of tert-butyl (2S)-2-(cyanomethyl)-4-[2-[[(2S,4R)-4-[2-[2-[2-[[[(2S,4R)-4-hydroxy-1-[(2S)-3-methyl-2-(3-methylisoxazol-5-yl)butanoyl]pyrrolidine-2-carbonyl]amino]methyl]-5-(4-methylthiazol-5-yl)phenoxy]ethoxy]ethoxy]-1-methyl-pyrrolidin-2-yl]methoxy]-7-(1-naphthyl)-6,8-dihydro-5H-pyrido[3,4-d]pyrimidin-4-yl]piperazine-1-carboxylate (60 mg, 0.051 mmol, 1 eq) in dichloromethane (6 mL) was added trifluoroacetic acid (2.31 g, 20.26 mmol, 1.5 mL, 400 eq). The mixture was stirred at 20° C. for 2 hours. The mixture was evaporated under vacuum to get the product (2S,4R)—N-[[2-[2-[2-[(3R,5S)-5-[[4-[(3S)-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide (50 mg) as a light yellow oil.

### Step 2: Preparation of (2S,4R)—N-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((S)-3-methyl-2-(3-methylisoxazol-5-yl)butanoyl)pyrrolidine-2-carboxamide

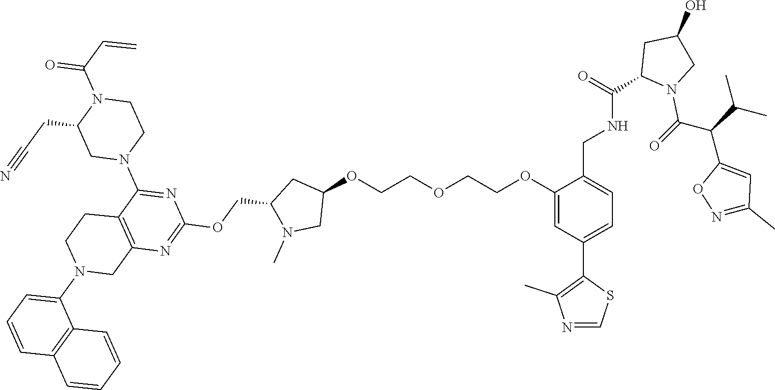 [SMILES: C=CC(=O)N1CCN(c2nc(OC[C@@H]3C[C@@H](OCCOCCOc4cc(-c5scnc5C)ccc4CNC(=O)[C@@H]4C[C@@H](O)CN4C(=O)[C@H](c4cc(C)no4)C(C)C)CN3C)nc3c2CCN(c2cccc4ccccc24)C3)C[C@@H]1CC#N]

To a solution of (2S,4R)—N-[[2-[2-[2-[(3R,5S)-5-[[4-[(3S)-3-(cyanomethyl)piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide (50 mg, 0.046 mmol, 1 eq) and 2,6-Lutidine (184 mg, 1.72 mmol, 0.2 mL, 37 eq) in the mixed solvent of dichloromethane (4 mL) and N,N-dimethylformamide (0.5 mL) was added prop-2-enoyl chloride (3.7 mg, 0.042 mmol, 0.9 eq) in dichloromethane (0.2 mL) in nitrogen. The mixture was stirred at −65° C. for 10 minutes. The mixture was quenched by water (10 mL) and extracted by dichloromethane (20 mL×3). The organic layers were combined and evaporated under vacuum to get a residue (50 mg). The residue was purified through Prep-HPLC. The product (2S,4R)—N-[[2-[2-[2-[(3R,5S)-5-[[4-[(3S)-3-(cyanomethyl)-4-prop-2-enoyl-piperazin-1-yl]-7-(1-naphthyl)-6,8-dihydro-5H-pyrido[3,4-d]pyrimidin-2-yl]oxymethyl]-1-methyl-pyrrolidin-3-yl]oxyethoxy]ethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide (28.5 mg, 0.024 mmol, 52% yield, 96% purity, trifluoroacetate) was obtained as an off-white solid. LC/MS (ESI) m/z: 1136.3 [M+1]⁺; ¹H-NMR (400 MHz, DMSO-d₆) δ 8.99 (s, 1H), 8.65-8.37 (m, 1H), 8.20-8.17 (m, 1H), 7.95-7.93 (m, 1H), 7.67 (d, J=8.0 Hz, 1H), 7.56-7.54 (m, 2H), 7.47 (t, J=7.6 Hz, 1H), 7.36 (d, J=7.6 Hz, 1H), 7.24-7.22 (m, 1H), 7.06-7.00 (m, 2H), 6.88 (s, 1H), 6.23-6.18 (m, 2H), 5.81-5.78 (m, 1H), 4.99-4.78 (m, 1H), 4.63-4.59 (m, 1H), 4.48-4.17 (m, 12H), 4.10-4.07 (m, 3H), 3.96-3.92 (m, 1H), 3.82-3.73 (m, 5H), 3.67-3.55 (m, 5H), 3.46-3.37 (m, 2H), 3.30-3.17 (m, 4H), 3.00-2.97 (m, 7H), 2.48-2.44 (m, 3H), 2.39-2.22 (m, 2H), 2.21-2.13 (m, 3H), 2.09-1.88 (m, 2H), 0.97-0.93 (m, 3H), 0.79-0.76 (m, 3H).

The following compounds can be prepared in an analogous manner to compound (2S,4R)—N-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((R)-3-methyl-2-(3-methylisoxazol-5-yl)butanoyl)pyrrolidine-2-carboxamide and (2S,4R)—N-(2-(2-(2-(((3R,5S)-5-(((4-((S)-4-acryloyl-3-(cyanomethyl)piperazin-1-yl)-7-(naphthalen-1-yl)-5,6,7,8-tetrahydropyrido[3,4-d]pyrimidin-2-yl)oxy)methyl)-1-methylpyrrolidin-3-yl)oxy)ethoxy)ethoxy)-4-(4-methylthiazol-5-yl)benzyl)-4-hydroxy-1-((S)-3-methyl-2-(3-methylisoxazol-5-yl)butanoyl)pyrrolidine-2-carboxamide .

### 1. (2S,4R)—N-[[2-[2-[(3R,5S)-5-[[4-[(3S)-3-(cyanomethyl)-4-prop-2-enoyl-piperazin-1-yl]- 7-(1-naphthyl)-6,8-dihydro-5H-pyrido[3,4-d]pyrimidin-2-yl]oxymethyl]-1-methyl-pyrrolidin-3-yl]oxyethoxy]-4-(4-methylthiazol-5-yl)phenyl]methyl]-4-hydroxy-1-[(2R)-3-methyl-2-(3-methylisoxazol-5-yl)butanoyl]pyrrolidine-2-carboxamide

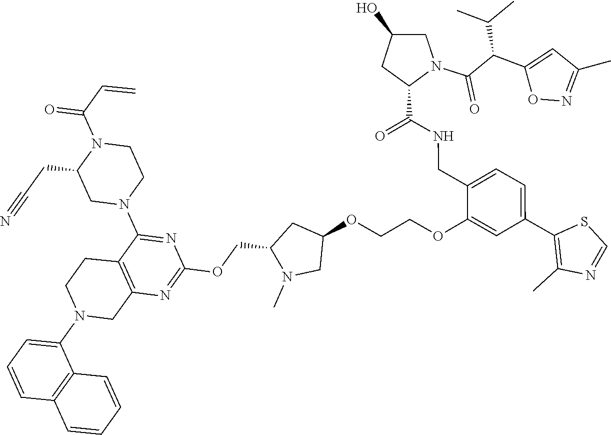 [SMILES: C=CC(=O)N1CCN(c2nc(OC[C@@H]3C[C@@H](OCCOc4cc(-c5scnc5C)ccc4CNC(=O)[C@@H]4C[C@@H](O)CN4C(=O)[C@@H](c4cc(C)no4)C(C)C)CN3C)nc3c2CCN(c2cccc4ccccc24)C3)C[C@@H]1CC#N]

(formic acid salt, white solid). LC/MS (ESI) m/z: 1092 [M+H]⁺. ¹H-NMR (400 MHz, DMSO-d₆) δ 8.99 (s, 1H), 8.69-8.33 (m, 1H), 8.32-8.25 (m, 1H), 8.22-8.14 (m, 1H), 7.88 (s, 1H), 7.65 (d, J=8.1 Hz, 1H), 7.59-7.51 (m, 2H), 7.47 (t, J=7.8 Hz, 1H), 7.39-7.33 (m, 1H), 7.39-7.16 (m, 1H), 7.11-6.98 (m, 2H), 6.96-6.75 (m, 1H), 6.30-5.92 (m, 2H), 5.82-5.73 (m, 1H), 5.06-4.74 (m, 1H), 4.50-4.33 (m, 2H), 4.32-4.22 (m, 3H), 4.18 (br t, J=4.5 Hz, 2H), 4.15 (br s, 2H), 4.13-4.07 (m, 2H), 4.03 (br d, J=11.4 Hz, 2H), 3.82-3.71 (m, 4H), 3.56 (br d, J=13.0 Hz, 1H), 3.43 (br d, J=9.6 Hz, 2H), 3.39 (br s, 1H), 3.33 (br d, J=3.5 Hz, 2H), 3.23-3.15 (m, 3H), 3.07-2.98 (m, 2H), 2.97-2.85 (m, 2H), 2.79-2.70 (m, 1H), 2.48-2.45 (m, 3H), 2.34 (s, 3H), 2.29-2.21 (m, 2H), 2.21-2.12 (m, 3H), 2.07-1.99 (m, 1H), 1.98-1.83 (m, 3H), 0.98-0.91 (m, 3H), 0.80-0.73 (m, 3H).

### 2. (2S,4R)—N-[[2-[2-[(3R,5S)-5-[[4-[(3S)-3-(cyanomethyl)-4-prop-2-enoyl-piperazin-1-yl]- 7-(1-naphthyl)-6,8-dihydro-5H-pyrido[3,4-d]pyrimidin-2-yl]oxymethyl]-1-methyl-pyrrolidin-3-yl]oxyethoxy]-4-(4-methylthiazol-5-yl)phenyl]methyl]-4-hydroxy-1-[(2S)-3-methyl-2-(3-methylisoxazol-5-yl)butanoyl]pyrrolidine-2-carboxamide

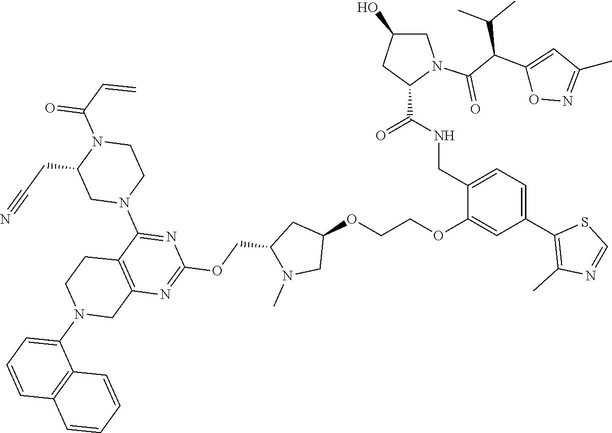 [SMILES: C=CC(=O)N1CCN(c2nc(OC[C@@H]3C[C@@H](OCCOc4cc(-c5scnc5C)ccc4CNC(=O)[C@@H]4C[C@@H](O)CN4C(=O)[C@H](c4cc(C)no4)C(C)C)CN3C)nc3c2CCN(c2cccc4ccccc24)C3)C[C@@H]1CC#N]

(formic acid salt, white solid). LC/MS (ESI) m/z: 1092 [M+H]⁺. ¹H-NMR (400 MHz, DMSO-d₆) δ 8.99 (s, 1H), 8.69-8.33 (m, 1H), 8.32-8.25 (m, 1H), 8.22-8.14 (m, 1H), 7.88 (s, 1H), 7.65 (d, J=8.1 Hz, 1H), 7.59-7.51 (m, 2H), 7.47 (t, J=7.8 Hz, 1H), 7.39-7.33 (m, 1H), 7.39-7.16 (m, 1H), 7.11-6.98 (m, 2H), 6.96-6.75 (m, 1H), 6.30-5.92 (m, 2H), 5.82-5.73 (m, 1H), 5.06-4.74 (m, 1H), 4.50-4.33 (m, 2H), 4.32-4.22 (m, 3H), 4.18 (br t, J=4.5 Hz, 2H), 4.15 (br s, 2H), 4.13-4.07 (m, 2H), 4.03 (br d, J=11.4 Hz, 2H), 3.82-3.71 (m, 4H), 3.56 (br d, J=13.0 Hz, 1H), 3.43 (br d, J=9.6 Hz, 2H), 3.39 (br s, 1H), 3.33 (br d, J=3.5 Hz, 2H), 3.23-3.15 (m, 3H), 3.07-2.98 (m, 2H), 2.97-2.85 (m, 2H), 2.79-2.70 (m, 1H), 2.48-2.45 (m, 3H), 2.34 (s, 3H), 2.29-2.21 (m, 2H), 2.21-2.12 (m, 3H), 2.07-1.99 (m, 1H), 1.98-1.83 (m, 3H), 0.98-0.91 (m, 3H), 0.80-0.73 (m, 3H).

### Exemplary Synthesis of (2S,4R)-1-[(2S)-2-[[2-[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 2-[(2S)-4-[6-chloro-8-fluoro-7-(3-hydroxy-1-naphthyl)-2-(2-oxoethoxy) quinazolin-4-yl]piperazin-2-yl]acetonitrile

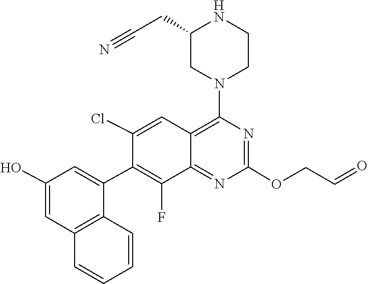 [SMILES: N#CC[C@H]1CN(c2nc(OCC=O)nc3c(F)c(-c4cc(O)cc5ccccc45)c(Cl)cc23)CCN1]

To a solution of tert-butyl (2S)-4-[6-chloro-2-(2,2-dimethoxyethoxy)-8-fluoro-7-(3-hydroxy-1-naphthyl)quinazolin-4-yl]-2-(cyanomethyl)piperazine-1-carboxylate (4.3 g, 6.59 mmol, 1 eq) in acetone (10 mL) was added aqueous HCl (12 M, 10 mL, 18.20 eq), and the reaction mixture was stirred at 25° C. for 1 hour. NaHCO₃ (9.97 g, 118.69 mmol, 4.62 mL, 18 eq) in H₂O (20 mL) was then added, and the resulting precipitate was filtered and dried under reduced pressure to afford 2-[(2S)-4-[6-chloro-8-fluoro-7-(3-hydroxy-1-naphthyl)-2-(2-oxoethoxy)quinazolin-4-yl]piperazin-2-yl]acetonitrile (2.6 g, 4.82 mmol, 73% yield) as a brown solid. LC/MS (ESI) m/z: 506.1 [M+H]⁺. ¹H-NMR (400 MHz, DMSO-d₆) δ 10.0-9.90 (m, 1H), 7.99-7.98 (m, 1H), 7.81-7.79 (m, 1H), 7.43-7.41 (m, 1H), 7.28-7.20 (m, 3H), 7.18-7.06 (m, 1H), 4.19-4.16 (m, 2H), 3.60-3.58 (m, 3H), 3.32-2.74 (m, 4H), 2.72-2. 54 (m, 2H).

### Step 2: Preparation of (2S,4R)-1-[(2S)-2-[[2-[[1-[2-[6-chloro-4-[(3S)-3-(cyanomethyl) piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

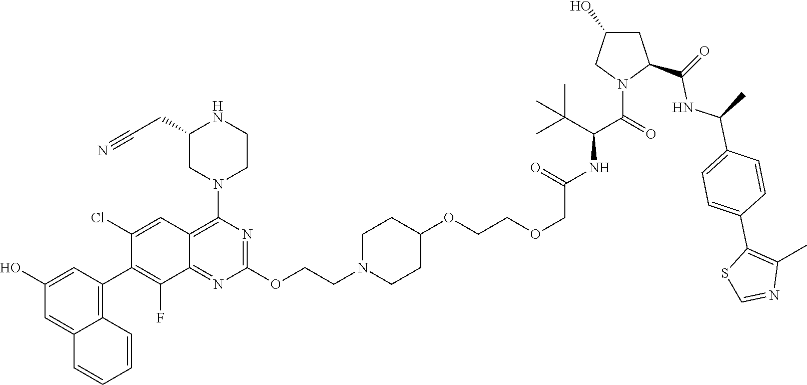 [SMILES: Cc1ncsc1-c1ccc([C@H](C)NC(=O)[C@@H]2C[C@@H](O)CN2C(=O)[C@@H](NC(=O)COCCOC2CCN(CCOc3nc(N4CCN[C@@H](CC#N)C4)c4cc(Cl)c(-c5cc(O)cc6ccccc56)c(F)c4n3)CC2)C(C)(C)C)cc1]

To a solution of (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (350 mg, 550.10 umol, 1 eq, HCl) in isopropanol (4 mL) was added NaOAc (225.64 mg, 2.75 mmol, 5 eq) followed by 2-[(2S)-4-[6-chloro-8-fluoro-7-(3-hydroxy-1-naphthyl)-2-(2-oxoethoxy)quinazolin-4-yl]piperazin-2-yl]acetonitrile (278.31 mg, 550.10 umol, 1 eq) in CH₂Cl₂ (4 mL), and the resulting mixture was stirred at 25° C. for 1 hour. 2-Methylpyridine borane (294.20 mg, 2.75 mmol, 5 eq) was then added, and the reaction mixture was stirred at 25° C. for 12 hours. The mixture was concentrated, and the resulting crude product was purified by prep-HPLC (22-52% CH₃CN in water (0.1% TFA)) to afford (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (320 mg, 265.84 umol, 48% yield, TFA salt) as a yellow solid. LC/MS (ESI) m/z: 545.8 [M/2+H]⁺.

### Step 3: Preparation of (2S,4R)-1-[(2S)-2-[[2-[2-[[1-[2-[6-chloro-4-[(3S)-3-(cyanomethyl)- 4-prop-2-enoyl-piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

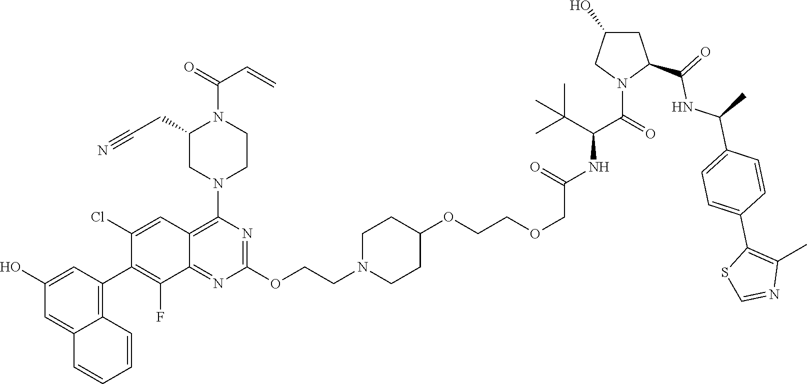 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

To a solution of (2S,4R)-1-[(2S)-2-[[2-[2-[[1-[2-[6-chloro-4-[(3S)-3-(cyanomethyl) piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (170 mg, 151.82 umol, 1 eq) in CH₂Cl₂ (2 mL) at −78° C. was added 2,6-dimethylpyridine (162.68 mg, 1.52 mmol, 176.82 uL, 10 eq) followed by prop-2-enoyl chloride (13.74 mg, 151.82 umol, 12.38 uL, 1 eq), and the reaction mixture was stirred at −78° C. for 10 minutes. CH₃OH (1 mL) was added, and the resulting mixture was concentrated. The resulting yellow solid was purified by prep-HPLC (20-50% CH₃CN in water (0.225% formic acid)) to give (2S,4R)-1-[(2S)-2-[[2-[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (104 mg, 84.41 umol, 56% yield, formic acid salt) as a yellow solid. LC/MS (ESI) m/z: 1173.5 [M+H]⁺. ¹H-NMR (400 MHz, CD₃OD) δ 8.86-8.88 (m, 1H), 8.53 (s, 1H), 8.10-8.09 (m, 1H), 7.75 (d, J=8.0 Hz, 1H), 7.44-7.39 (m, 6H), 7.39-7.21 (m, 3H), 7.05-7.04 (m, 1H), 7.03-6.81 (m, 1H), 6.35-6.30 (m 1H), 5.88-5.84 (m, 1H), 5.10-4.98 (m, 2H), 4.73-4.71 (m, 2H), 4.57-4.51 (m, 4H), 4.07-4.06 (m, 2H), 3.86-3.60 (m, 12H), 3.20-3.15 (m, 4H), 3.15-2.60 (m, 3H), 2.46 (s, 3H), 2.24-2.22 (m, 1H), 2.05-1.86 (m, 5H), 1.58-1.48 (m, 3H), 1.05-1.03 (m, 9H).

The following compound can be prepared in an analogous manner to (2S,4R)-1-[(2S)-2-[[2-[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4S)-1-((2S)-2-(2-(2-((1-(2-((4-((S)-4-acryloyl-3-(cyanomethyl)piperazin-1-yl)-6-chloro-8-fluoro-7-(3-hydroxynaphthalen-1-yl)quinazolin-2-yl)oxy)ethyl)piperidin-4-yl)oxy)ethoxy)acetamido)-3,3-dimethylbutanoyl)-4-hydroxy-N—((S)-1-(4-(4-methylthiazol-5-yl)phenyl)ethyl)pyrrolidine-2-carboxamide

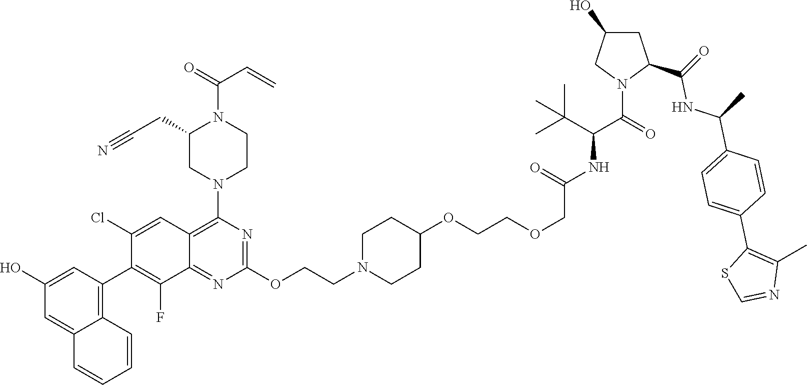 [SMILES: C=CC(=O)N1CCN(c2nc(OCCN3CCC(OCCOCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(white solid, formic acid.) LC/MS (ESI) m/z: 1173.4 [M+H]⁺. ¹H-NMR (400 MHz, DMSO-d₆) δ 10.00 (s, 1H), 8.99-8.95 (m, 1H), 8.40-8.33 (m, 1H), 8.15-8.08 (m, 2H), 7.84-7.78 (m, 1H), 7.47-7.27 (m, 8H), 7.24-7.13 (m, 2H), 7.08-7.04 (m, 1H), 6.98-6.75 (m, 1H), 6.25-6.17 (m, 1H), 5.83-5.73 (m, 1H), 5.35-5.30 (m, 1H), 5.01-4.84 (m, 2H), 4.54-4.46 (m, 2H), 4.40-4.29 (m, 3H), 4.28-4.08 (m, 3H), 3.96-3.93 (m, 2H), 3.90-3.80 (m, 2H), 3.66-3.50 (m, 7H), 3.42-3.37 (m, 2H), 3.29 (br s, 6H), 3.09-2.99 (m, 2H), 2.44 (s, 3H), 1.95-1.73 (m, 3H), 1.67-1.59 (m, 1H), 1.38-1.33 (m, 3H), 0.95-0.90 (m, 9H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of (2S,4R)-1-[(2S)-2-[[2-[[1-[2-[6-chloro-4-[(3S)-3-(cyanomethyl) piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

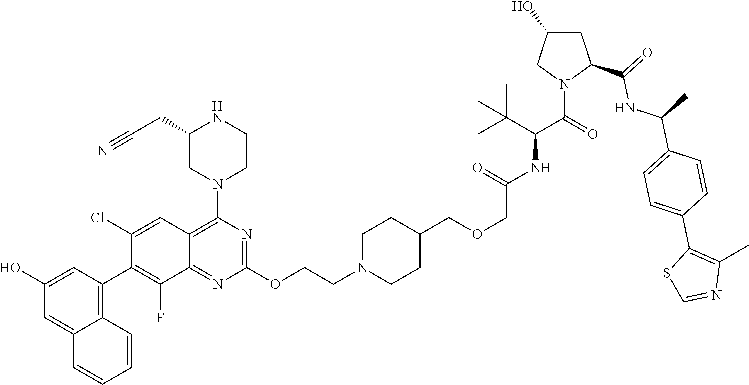 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCN[C@@H](CC#N)C4)c4cc(Cl)c(-c5cc(O)cc6ccccc56)c(F)c4n3)CC2)C(C)(C)C)cc1]

To 2-[(2S)-4-[6-chloro-8-fluoro-7-(3-hydroxy-1-naphthyl)-2-(2-oxoethoxy) quinazolin-4-yl]piperazin-2-yl]acetonitrile (500 mg, 988.28 umol, 1 eq) in isopropanol (10 mL) was added NaOAc (405.36 mg, 4.94 mmol, 5 eq) followed by (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (628.79 mg, 988.28 umol, 1 eq, HCl) in CH₂Cl₂ (10 mL), and the resulting mixture was stirred at 25° C. for 1 hour. 2-Methylpyridine borane (528.54 mg, 4.94 mmol, 5 eq) was then added, and the reaction mixture was stirred at 25° C. for 12 hours. The mixture was concentrated under reduced pressure, and the resulting residue was purified by column chromatography on SiO₂ (petroleum ether/EtOAc=1/1 to CH₂Cl₂/CH₃OH=5/1) followed by prep-HPLC (13-43% CH₃CN in water (0.1% TFA)) to afford (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (210 mg, 192.71 umol, 20% yield) as a yellow solid. LC/MS (ESI) m/z: 1089.6 [M+H]⁺.

### Step 2: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

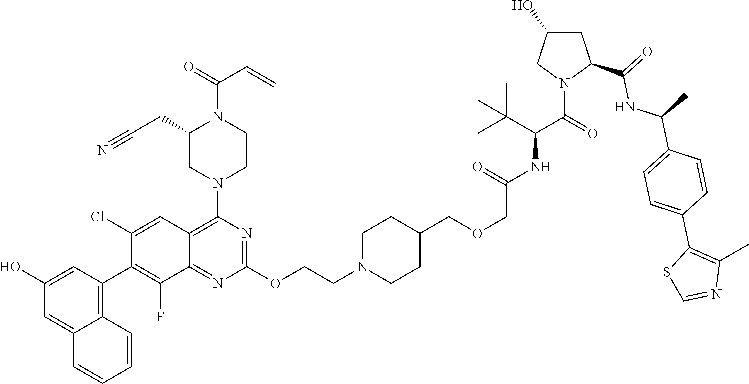 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

To a mixture of (2S,4R)-1-[(2S)-2-[[2-[[1-[2-[6-chloro-4-[(3S)-3-(cyanomethyl) piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (210 mg, 192.71 umol, 1 eq) and 2,6-lutidine (206.50 mg, 1.93 mmol, 224.45 uL, 10 eq) in CH₂Cl₂ (3 mL) and DMF (3 mL) at −78° C. was added prop-2-enoyl chloride (17.44 mg, 192.71 umol, 15.71 uL, 1 eq), and the reaction mixture was stirred at −78° C. for 0.5 hours. The mixture was diluted with water (20 mL) and extracted with CH₂Cl₂ (3×25 mL). The combined organic extracts were washed with brine (3×30 mL), dried over anhydrous Na₂SO₄, filtered, and concentrated under reduced pressure. The resulting residue was purified by prep-HPLC (20-50% CH₃CN water (0.225% formic acid)) to afford (2S,4R)-1-[(2S)-2-[[2-[[1-[2-[6-chloro-4-[(3S)-3-(cyanomethyl)-4-prop-2-enoyl- piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (192.3 mg, 151.32 umol, 79% yield, formic acid salt) as a white solid. LC/MS (ESI) m/z: 1143.1 [M+H]⁺. ¹H-NMR (400 MHz, DMSO-d₆) δ 10.0 (s, 1H), 8.98 (s, 1H), 8.43 (m, 1H), 8.21 (s, 1H), 8.11 (s, 1H), 7.90-7.80 (m, 1H), 7.45-7.22 (m, 10H), 7.08-7. 07 (1H), 6.24-6.19 (m, 1H), 5.81-5.78 (m, 1H), 5.25-4.85 (m, 4H), 4.60-4.25 (m, 9H), 4.10-4.00 (m, 1H), 3.80 (s, 3H), 3.63-3.54 (m, 3H), 3.00-2.90 (m, 2H), 2.75-2.65 (m, 2H), 2.46 (s, 3H), 2.10-1.90 (m, 3H), 1.80-1.45 (m, 5H), 1.36-1.34 (m, 3H), 1.20-1.10 (m, 2H), 0.93-0.92 (m, 10H).

The following compound can be prepared in an analogous manner to (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl) quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4S)-1-((2S)-2-(2-((1-(2-((4-((S)-4-acryloyl-3-(cyanomethyl)piperazin-1-yl)-6-chloro-8-fluoro-7-(3-hydroxynaphthalen-1-yl)quinazolin-2-yl)oxy)ethyl)piperidin-4-yl)methoxy)acetamido)-3,3-dimethylbutanoyl)-4-hydroxy-N—((S)-1-(4-(4-methylthiazol-5-yl)phenyl)ethyl)pyrrolidine-2-carboxamide

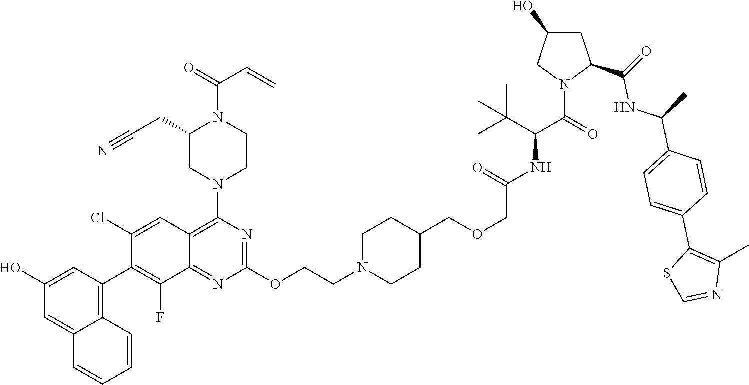 [SMILES: C=CC(=O)N1CCN(c2nc(OCCN3CCC(COCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(white solid, formic acid). LC/MS (ESI) m/z: 1143.4 [M+H]⁺. ¹H-NMR (400 MHz, DMSO-d₆) δ 10.04-10.00 (m, 1H), 8.99-8.96 (m, 1H), 8.41 (s, 1H), 8.15-8.10 (m, 1H), 7.83-7.76 (m, 1H), 7.46-7.35 (m, 6H), 7.32-7.26 (m, 2H), 7.25-7.12 (m, 3H), 7.08-7.04 (m, 1H), 6.76 (s, 1H), 6.25-6.18 (m, 1H), 5.83-5.77 (m, 1H), 5.33-5.28 (m, 1H), 5.03-4.83 (m, 2H), 4.67-4.46 (m, 3H), 4.41-4.30 (m, 3H), 4.28-4.05 (m, 3H), 3.97-3.50 (m, 8H), 3.42-3.36 (m, 2H), 3.29 (br s, 4H), 3.11-2.99 (m, 3H), 2.45 (s, 3H), 1.88-1.57 (m, 5H), 1.37-1.34 (m, 3H), 0.94 (s, 9H).

### Exemplary Synthesis of (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

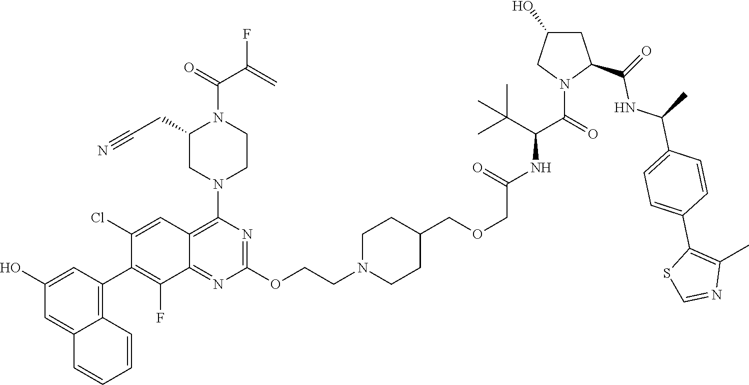 [SMILES: C=C(F)C(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

To a solution of (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (640 mg, 531.68 umol, 1 eq, TFA) in DMF (3 mL) were added diisopropylethylamine (206.15 mg, 1.60 mmol, 277.83 uL, 3 eq) and HATU (606.48 mg, 1.60 mmol, 3 eq) followed by 2-fluoroprop-2-enoic acid (57.46 mg, 638.01 umol, 1.2 eq) dropwise, and the reaction mixture was stirred at 20° C. for 2 hours. The mixture was poured onto saturated aqueous K₂CO₃ (30 mL) and, after stirred for 30 minutes, the mixture was extracted with EtOAc/THF (40 mL, 1/1). The organic extract was dried over anhydrous Na₂SO₄, filtered, and the filtrate concentrated. The resulting crude product was purified by prep-HPLC (21-51% CH₃CN in water (0.225% formic acid)). Further purification of impure fractions by prep-HPLC (28-58% CH₃CN in water (0.225% formic acid)) afforded (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (231.4 mg, 173.03 umol, 33% yield, formic acid salt) as a pale yellow solid. LC/MS (ESI) m/z: 1161.3 [M+H]⁺. ¹H-NMR (400 MHz, DMSO-d₆) δ 10.03 (s, 1H), 8.97 (s, 1H), 8.70-8.43 (m, 1H), 8.10 (s, 1H), 7.80-7.79 (m, 1H), 7.35-7.07 (m, 9H), 7.06 (s, 1H), 5.44-5.38 (m, 2H), 5.18-5.00 (m, 1H), 4.95-4.75 (m, 2H), 4.55-4.05 (m, 8H), 3.91 (s, 3H), 3.58-3.56 (m, 4H), 3.20-2.90 (m, 5H), 2.80-2.70 (m 2H), 2.50 (s, 3H), 2.44-2.06 (m, 3H), 1.70-1.10 (m, 9H), 0.92 (s, 9H).

### Exemplary Synthesis of (2S,4R)-1-[(2S)-2-[[2-[2-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl (2S)-4-[6-chloro-8-fluoro-2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]-2-(cyanomethyl)piperazine-1-carboxylate

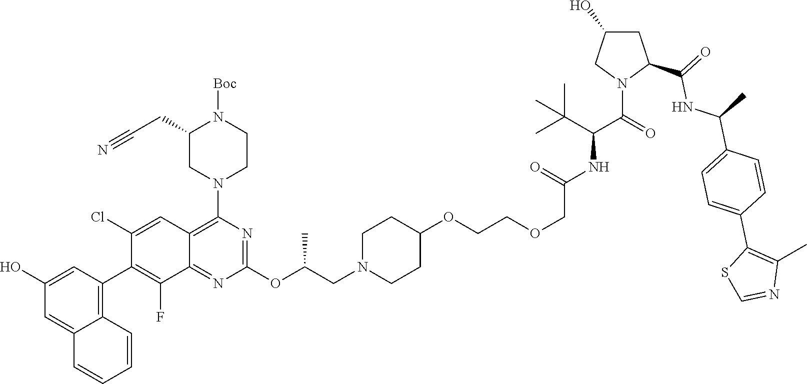 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc(N4CCN(C(=O)OC(C)(C)C)[C@@H](CC#N)C4)c4cc(Cl)c(-c5cc(O)cc6ccccc56)c(F)c4n3)CC2)C(C)(C)C)cc1]

To a solution of tert-butyl (2S)-4-[6-chloro-8-fluoro-7-(3-hydroxy-1-naphthyl)-2-[(1R)-1-methyl-2-oxo-ethoxy]quinazolin-4-yl]-2-(cyanomethyl)piperazine-1-carboxylate (40 mg, 0.06 mmol, 1.00 eq) and (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (47 mg, 0.07 mmol, 1.10 eq, hydrochloride salt) in a mixture of CH₂Cl₂ (0.5 mL) and CH₃OH (0.5 mL) was added NaOAc (64 mg, 0.77 mmol, 12.00 eq), and the resulting mixture was stirred at 20° C. for 1 hour. 2-Methylpyridine borane (35 mg, 0.32 mmol, 5.00 eq) was then added, and the reaction mixture was stirred at 40° C. for 12 hours. The solvent was removed under reduced pressure, and the resulting residue was purified by prep-thin layer chromatography (CH₂Cl₂:CH₃OH=20:1) to afford tert-butyl (2S)-4-[6-chloro-8-fluoro-2-[(1R)-2-[4-[2-[2-[[(1S)-1-[(2S,4R)-4-hydroxy-2-[[(1S)-1-[4-(4- methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]-2-(cyanomethyl)piperazine-1-carboxylate (25 mg, 0.02 mmol, 31% yield) as a yellow solid. LC/MS (ESI) m/z: 1233.6 [M+H]⁺.

### Step 2: Preparation of (2S,4R)-1-[(2S)-2-[[2-[2-[[1-[(2R)-2-[6-chloro-4-[(3S)-3-(cyanomethyl)piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

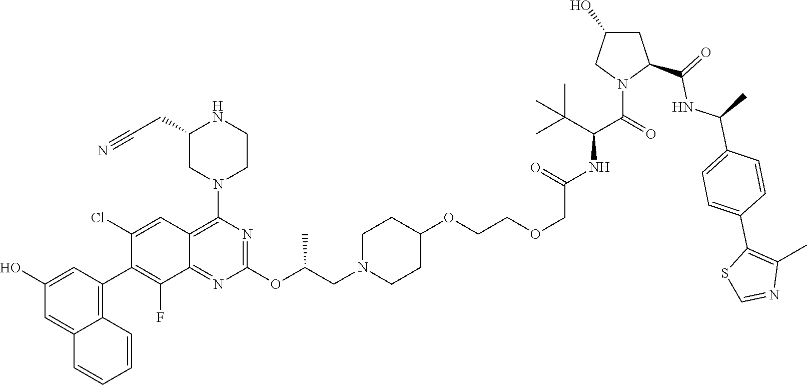 [SMILES: Cc1ncsc1-c1ccc([C@H](C)NC(=O)[C@@H]2C[C@@H](O)CN2C(=O)[C@@H](NC(=O)COCCOC2CCN(C[C@@H](C)Oc3nc(N4CCN[C@@H](CC#N)C4)c4cc(Cl)c(-c5cc(O)cc6ccccc56)c(F)c4n3)CC2)C(C)(C)C)cc1]

To a solution of tert-butyl (2S)-4-[6-chloro-8-fluoro-2-[(1R)-2-[4-[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]-1-methyl-ethoxy]-7-(3-hydroxy-1-naphthyl)quinazolin-4-yl]-2-(cyanomethyl)piperazine-1-carboxylate (25 mg, 0.02 mmol, 1.00 eq) in CH₂Cl₂ (0.6 mL) was added trifluoroacetic acid (770 mg, 6.75 mmol, 0.5 mL, 333.30 eq), and the reaction mixture was stirred at 20° C. for 1 hour. The solvent was removed under reduced pressure to afford (2S,4R)-1-[(2S)-2-[[2-[2-[[1-[(2R)-2-[6-chloro-4-[(3S)-3-(cyanomethyl)piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (25 mg, 0.02 mmol, 91% yield, TFA salt) as a yellow solid. LC/MS (ESI) m/z: 1133.6 [M+H]⁺.

### Step 3: Preparation of (2S,4R)-1-[(2S)-2-[[2-[2-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

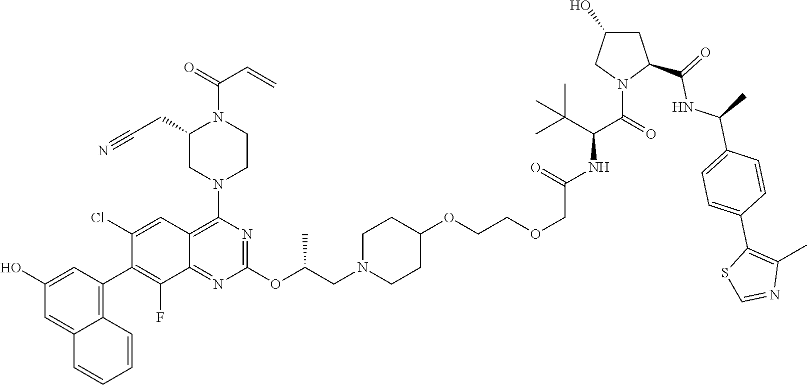 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

To a solution of (2S,4R)-1-[(2S)-2-[[2-[2-[[1-[(2R)-2-[6-chloro-4-[(3S)-3-(cyanomethyl)piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (25 mg, 0.02 mmol, 1.00 eq, TFA salt) and 2,6-lutidine (39 mg, 0.37 mmol, 20.00 eq) in CH₂Cl₂ (3 mL) at −70° C. was added a solution of prop-2-enoyl chloride (2 mg, 0.02 mmol, 1.05 eq) in CH₂Cl₂ (0.2 mL), and the reaction mixture was stirred at −70° C. for 0.5 hours. The solvent was removed under reduced pressure, and the resulting residue was purified by prep-thin layer chromatography (CH₂Cl₂:CH₃OH=10:1) followed by semi-preparative reverse phase HPLC (38-58% CH₃CN in water (10 mmol NH₄HCO₃)) to afford (2S,4R)-1-[(2S)-2-[[2-[2-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (5.9 mg, 0.005 mmol, 26% yield) as a yellow solid. LC/MS (ESI) m/z: 1187.6 [M+H]⁺. ¹H-NMR (400 MHz, CD₃OD) δ 8.88 (s, 1H), 8.06 (s, 1H), 7.75 (d, J=8.4 Hz, 1H), 7.55-7.37 (m, 5H), 7.30-7.16 (m, 3H), 7.05 (t, J=2.4 Hz, 1H), 7.00-6.75 (m, 1H), 7.32 (d, J=16.8 Hz, 1H), 5.85 (d, J=10.0 Hz, 1H), 5.70-5.55 (m, 1H), 5.20-4.95 (m, 2H), 4.65-4.52 (m, 3H), 4.50-4.35 (m, 3H), 4.25-4.10 (m, 1H), 4.10-3.98 (m, 2H), 3.85-3.75 (m, 2H), 3.75 (dd, J=3.6, 11.2 Hz, 1H), 3.70-3.58 (m, 5H), 3.45-3.35 (m, 1H), 3.10-2.75 (m, 5H), 2.63-2.51 (m, 1H), 2.48 (d, J=2.0 Hz, 3H), 2.45-2.25 (m, 2H), 2.25-2.15 (m, 1H), 2.14-1.82 (m, 3H), 1.62-1.45 (m, 5H), 1.40 (d, J=6.4 Hz, 3H), 1.03 (s, 9H).

The following compound can be prepared in an analogous manner to (2S,4R)-1-[(2S)-2-[[2-[2-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### 1. (2S,4R)-1-[(2S)-2-[[2-[2-[[1-[2-[6-chloro-4-[(3S)-3-(cyanomethyl)-4-prop-2- enoyl-piperazin-1-yl]-8-fluoro-7-(8-methyl-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

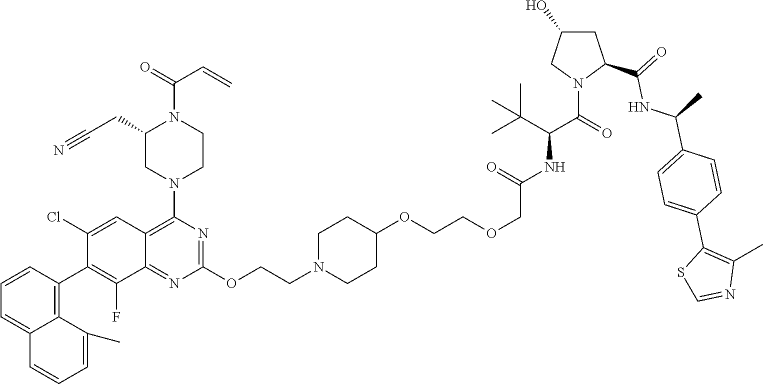 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cccc5cccc(C)c45)c(Cl)cc23)C[C@@H]1CC#N]

(formic acid salt, white solid). LC/MS (ESI) m/z: 1171.6 [M+H]⁺. ¹H-NMR (400 MHz, CD₃OD) δ 8.93-8.80 (m, 1H), 8.53 (dd, J=2.4, 3.2 Hz, 1H), 8.07-7.96 (m, 2H), 7.84 (d, J=7.6 Hz, 1H), 7.55 (dd, J=2.4, 7.6 Hz, 1H), 7.44-7.34 (m, 5H), 7.26 (d, J=7.2 Hz, 2H), 6.94-6.73 (m, 1H), 6.31 (d, J=16.4 Hz, 1H), 5.84 (d, J=10.4 Hz, 1H), 5.18-5.04 (m, 1H), 5.03-4.92 (m, 2H), 4.85-4.82 (m, 2H), 4.69 (s, 2H), 4.63-4.30 (m, 4H), 4.05 (s, 2H), 3.83 (d, J=11.6 Hz, 2H), 3.78-3.62 (m, 6H), 3.61-3.40 (m, 2H), 3.27-2.90 (m, 6H), 2.80-2.69 (m, 1H), 2.45 (s, 3H), 2.27-2.13 (m, 1H), 2.07 (d, J=5.6 Hz, 3H), 2.03-1.90 (m, 3H), 1.89-1.70 (m, 2H), 1.62-1.42 (m, 3H), 1.07-0.97 (m, 9H).

### Exemplary Synthesis of (2S,4R)-1-[(2R)-2-[3-[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of 2-42S)-1-acryloyl-4-(6-chloro-8-fluoro-7-(3-hydroxynaphthalen-1-yl)-2-(2-oxoethoxy)quinazolin-4-yl)piperazin-2-yl)acetonitrile

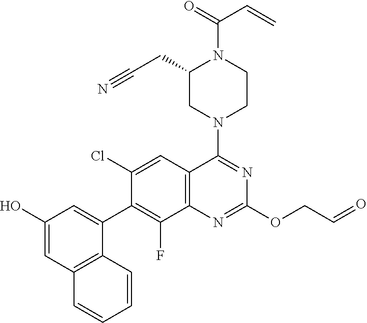 [SMILES: C=CC(=O)N1CCN(c2nc(OCC=O)nc3c(F)c(-c4cc(O)cc5ccccc45)c(Cl)cc23)C[C@@H]1CC#N]

To a mixture of 2-[(2S)-4-[6-chloro-8-fluoro-7-(3-hydroxy-1-naphthyl)-2-(2-oxoethoxy) quinazolin-4-yl]piperazin-2-yl]acetonitrile (1.2 g, 2.21 mmol, 1 eq, HCl) and 2,6-lutidine (711.21 mg, 6.64 mmol, 773.05 uL, 3 eq) in DMF (2 mL) and CH₂Cl₂ (8 mL) at −78° C. was added prop-2-enoyl chloride (180.22 mg, 1.99 mmol, 162.36 uL, 0.9 eq) dropwise, and the reaction mixture was stirred at −78° C. for 10 minutes. Water (5 mL) was added, and the resulting mixture was extracted with CH₂Cl₂ (2×20 mL). The organic extract was dried over anhydrous Na₂SO₄, filtered, and the filtrate concentrated. The resulting crude product was purified by column chromatography on SiO₂ (0-100% EtOAc in petroleum ether) followed by prep-HPLC (33-63% CH₃CN in water (0.225% formic acid)) to afford 2-[(2S)-4-[6-chloro-8-fluoro-7-(3-hydroxy-1-naphthyl)-2-(2-oxoethoxy) quinazolin-4-yl]-1-prop-2-enoyl-piperazin-2-yl]acetonitrile (570 mg, 851.99 umol, 39% yield) as a brown solid. LC/MS (ESI) m/z: 560.1 [M+H]⁺. ¹H-NMR (400 MHz, CDCl₃) δ 9.79 (s, 1H), 7.84 (s, 1H), 7.77-7.75 (m, 1H), 7.33-7.23 (m, 5H), 7.22-7.10 (m 1H), 6.65-6.60 (m, 1H), 6.45-6.41 (m, 1H), 5.87 (d, J=8.0 Hz, 1H), 5.04-4.99 (m, 2H), 4.39-4.33 (m, 2H), 4.00-3.64 (m, 4H), 3.10-2.90 (m, 2H).

### Step 2: Preparation of tert-butyl 4-(2-hydroxyethoxy) piperidine-1-carboxylate

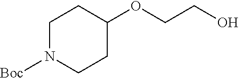 [SMILES: CC(C)(C)OC(=O)N1CCC(OCCO)CC1]

To a solution of tert-butyl 4-(2-benzyloxyethoxy)piperidine-1-carboxylate (50 g, 149.06 mmol, 1.00 eq) in CH₃OH (500 mL) was added Pd/C (5 g, 10% purity) under N₂, and the reaction mixture was stirred at 50° C. for 12 hours under H₂ atmosphere. The mixture was filtered, and the filtrate concentrated under reduced pressure. Purification by chromatography on SiO₂ (10%-100% EOAc in petroleum ether followed by 2-10% CH₃OH in EtOAc) afforded tert-butyl 4-(2-hydroxyethoxy)piperidine-1-carboxylate (32 g, 130.44 mmol, 88% yield) as a yellow oil. ¹H-NMR (400 MHz, CDCl₃) δ 3.84-3.67 (m, 4H), 3.65-3.42 (m, 3H), 3.06 (m, 2H), 1.91-1.74 (m, 2H), 1.56-1.47 (m, 2H), 1.44 (s, 9H).

### Step 3: Preparation of tert-butyl 4-[2-[5-(1-methoxycarbonyl-2-methyl-propyl)isoxazol-3-yl]oxyethoxy]piperidine-1-carboxylate

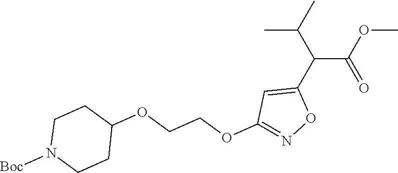 [SMILES: COC(=O)C(c1cc(OCCOC2CCN(C(=O)OC(C)(C)C)CC2)no1)C(C)C]

To a solution of tert-butyl 4-(2-hydroxyethoxy)piperidine-1-carboxylate (18 g, 72.29 mmol, 1.20 eq) and methyl 2-(3-hydroxyisoxazol-5-yl)-3-methyl-butanoate (12 g, 60.24 mmol, 1.00 eq) in THF (30 mL) at 0° C. was added Ph₃P (19 g, 72.29 mmol, 1.20 eq) followed by (E)-diisopropyl diazene-1,2-dicarboxylate (15 g, 72.29 mmol, 15 mL, 1.20 eq) dropwise, and the reaction mixture was stirred at 25° C. for 12 hours. The mixture was concentrated under reduced pressure, and the remaining material was purified by chromatography on SiO₂ (10-100% EtOAc in petroleum ether) to afford tert-butyl 4-[2-[5-(1-methoxycarbonyl-2-methyl-propyl)isoxazol-3-yl]oxyethoxy]piperidine-1-carboxylate (18 g, 42.20 mmol, 70% yield) as a yellow oil. ¹H-NMR (400 MHz, CDCl₃) δ 5.92 (s, 1H), 4.45-4.28 (m, 1H), 4.46-4.26 (m, 1H), 3.84-3.74 (m, 4H), 3.73 (s, 3H), 3.60-3.42 (m, 2H), 3.08 (m, 2H), 2.35 (td, J=6.8, 8.4 Hz, 1H), 1.92-1.73 (m, 2H), 1.55 (dd, J=4.0, 8.8 Hz, 2H), 1.46 (s, 9H), 1.00 (d, J=6.8 Hz, 3H), 0.92 (d, J=6.8 Hz, 3H)

### Step 4: Preparation of 2-[3-[2-[(1-tert-butoxycarbonyl-4-piperidyl) oxy]ethoxy]isoxazol-5-yl]-3-methyl-butanoic Acid

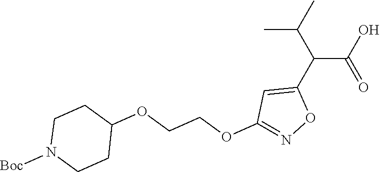 [SMILES: CC(C)C(C(=O)O)c1cc(OCCOC2CCN(C(=O)OC(C)(C)C)CC2)no1]

To a solution of tert-butyl 4-[2-[5-(1-methoxycarbonyl-2-methyl-propyl) isoxazol-3-yl]oxyethoxy]piperidine-1-carboxylate (18 g, 42.20 mmol, 1.00 eq) in THF (10 mL), CH₃OH (5 mL) and water (5 mL) was added LiOH hydrate (2.8 g, 63.31 mmol, 1.50 eq), and the reaction mixture was stirred at 25° C. for 2 hours. The solution pH was adjusted to 5 by addition of aqueous HCl, and the resulting mixture was extracted with CH₂Cl₂ (2×200 mL). The combined organic extracts were concentrated under reduced pressure to afford 2-[3-[2-[(1-tert-butoxycarbonyl-4-piperidyl)oxy]ethoxy]isoxazol-5-yl]-3-methyl-butanoic acid (17 g, 41.21 mmol, 98% yield) was obtained as a yellow oil.

### Step 5: Preparation of tert-butyl 4-[2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyethoxy]piperidine-1-carboxylate

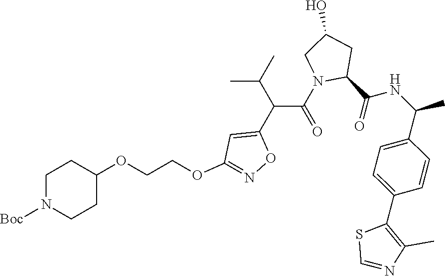 [SMILES: Cc1ncsc1-c1ccc([C@H](C)NC(=O)[C@@H]2C[C@@H](O)CN2C(=O)C(c2cc(OCCOC3CCN(C(=O)OC(C)(C)C)CC3)no2)C(C)C)cc1]

To a solution of 2-[3-[2-[(1-tert-butoxycarbonyl-4-piperidyl)oxy]ethoxy]isoxazol-5-yl]-3-methyl-butanoic acid (3.70 g, 8.97 mmol, 1.10 eq) and (2S,4R)-4-hydroxy-N-[(1S)-1-[4-(4-methylthiazol-5-yl)phenyl]ethyl]pyrrolidine-2-carboxamide (3 g, 8.15 mmol, 1.00 eq, hydrochloride salt) in DMF (30 mL) were added HATU (4.7 g, 12.23 mmol, 1.50 eq) and diisopropylethylamine (3.2 g, 24.46 mmol, 5 mL, 3.00 eq), and the reaction mixture was stirred at 25° C. for 12 hours. The solution was concentrated under reduced pressure, and the remaining material was purified by chromatography on SiO₂ (10-100% EtOAc in petroleum ether) to afford tert-butyl 4-[2-[5-[1-[(2S,4R)-4-hydroxy-2-[[(1S)-1-[4-(4-methylthiazol-5-yl) phenyl]ethyl]carbamoyl]pyrrolidine-1-carbonyl]-2-methyl-propyl]isoxazol-3-yl]oxyethoxy]piperidine-1-carboxylate (5.5 g, 7.58 mmol, 93% yield) as a yellow oil. The diastereomeric mixture was separated by SFC (40% isopropanol in water (0.1% NH₄OH)) to afford tert-butyl 4-[2-[5-[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]oxyethoxy]piperidine-1-carboxylate (2.2 g, 2.91 mmol, 22% yield) as a yellow solid and tert-butyl 4-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyethoxy]piperidine-1-carboxylate (1.5 g, 1.98 mmol, 15% yield) as a yellow solid. ¹H-NMR (400 MHz, CDCl₃)

### Step 6: Preparation of (2S,4R)-4-hydroxy-1-[(2R)-3-methyl-2-[3-[2-(4-piperidyloxy)ethoxy]isoxazol-5-yl]butanoyl]-N-[(1S)-1-[4-(4-methylthiazol-5-yl)phenyl]ethyl]pyrrolidine-2-carboxamide

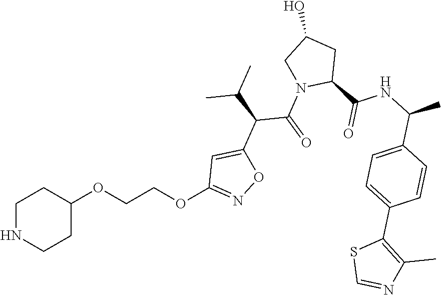 [SMILES: Cc1ncsc1-c1ccc([C@H](C)NC(=O)[C@@H]2C[C@@H](O)CN2C(=O)[C@@H](c2cc(OCCOC3CCNCC3)no2)C(C)C)cc1]

To a solution of tert-butyl 4-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyethoxy]piperidine-1-carboxylate (1.5 g, 2.07 mmol, 1.00 eq) in CH₂Cl₂ (10 mL) was added HCl (4N in dioxane, 10 mL), and the reaction mixture was stirred at 25° C. for 1 hour. The solution was concentrated under reduced pressure to afford (2S,4R)-4-hydroxy-1-[(2R)-3-methyl-2-[3-[2-(4-piperidyloxy)ethoxy]isoxazol-5-yl]butanoyl]-N-[(1S)-1-[4-(4-methylthiazol-5-yl)phenyl]ethyl]pyrrolidine-2-carboxamide (1.3 g, 1.96 mmol, 95% yield, HCl salt) as a yellow solid. ¹H-NMR (400 MHz, CD₃OD) δ 10.03 (d, J=2.8 Hz, 1H), 7.56 (q, J=8.0 Hz, 4H), 6.11-5.95 (m, 1H), 5.05 (q, J=7.2 Hz, 1H), 4.53 (t, J=8.4 Hz, 1H), 4.47-4.27 (m, 3H), 3.88-3.67 (m, 5H), 3.62 (s, 1H), 3.56-3.44 (m, 1H), 3.36-3.31 (m, 1H), 3.30-3.22 (m, 1H), 3.20-3.05 (m, 2H), 2.62 (s, 3H), 2.45-2.29 (m, 1H), 2.22 (dd, J=7.6, 13.1 Hz, 1H), 2.09-1.82 (m, 5H), 1.66-1.58 (m, 1H), 1.53 (d, J=7.2 Hz, 2H), 1.05 (d, J=6.8 Hz, 3H), 0.89 (d, J=6.8 Hz, 3H).

### Step 7: Preparation of (2S,4R)-1-[(2R)-2-[3-[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

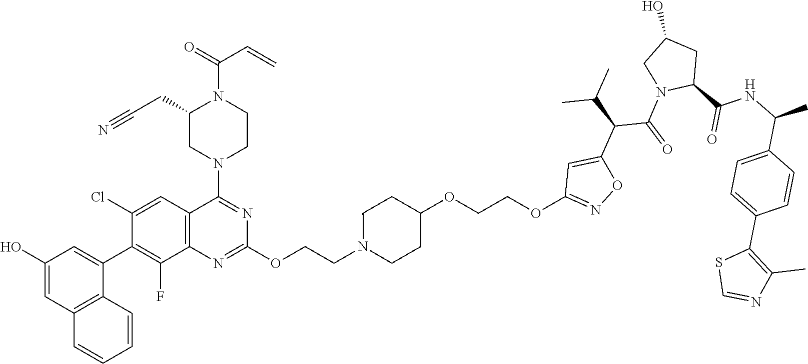 [SMILES: C=CC(=O)N1CCN(c2nc(OCCN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

To a solution of (2S,4R)-4-hydroxy-1-[(2R)-3-methyl-2-[3-[2-(4-piperidyloxy) ethoxy]isoxazol-5-yl]butanoyl]-N-[(1S)-1-[4-(4-methylthiazol-5-yl)phenyl]ethyl]pyrrolidine-2-carboxamide (390 mg, 0.59 mmol, 1.10 eq, HCl salt) and 2-[(2S)-4-[6-chloro-8-fluoro-7-(3-hydroxy-1-naphthyl)-2-(2-oxoethoxy)quinazolin-4-yl]-1-prop-2-enoyl-piperazin-2-yl]acetonitrile (300 mg, 0.54 mmol, 1.00 eq) in CH₂Cl₂ (5 mL) and CH₃OH (5 mL) at 0° C. were added NaOAc (140 mg, 1.61 mmol, 3.00 eq) and acetic acid (100 mg, 1.61 mmol, 3.00 eq) followed by NaBH₃CN (101 mg, 1.61 mmol, 3.00 eq), and the reaction mixture was stirred at 25° C. for 12 hours. The mixture was concentrated under reduced pressure, and the remaining material was purified by chromatography on SiO₂ (10-100% EtOAc in petroleum ether followed by 2-10% CH₃OH in CH₂Cl₂)). Further purification by HPLC (30-50% CH₃CN in water (0.225% formic acid)) afforded (2S,4R)-1-[(2R)-2-[3-[2-[[1-[2-[6-chloro-4-[(3S)-3-(cyanomethyl)-4-prop-2-enoyl- piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (151.3 mg, 0.12 mmol, 23% yield, formic acid salt) as a white solid. LC/MS (ESI) m/z: 1169.7 [M+H]⁺. ¹H-NMR (400 MHz, CD₃OD) δ 8.92-8.78 (m, 1H), 8.50 (d, J=2.0 Hz, 1H), 8.08 (s, 1H), 7.75 (d, J=8.4 Hz, 1H), 7.50-7.33 (m, 5H), 7.32-7.11 (m, 3H), 7.04 (d, J=2.0 Hz, 1H), 6.96-6.71 (m, 1H), 6.31 (d, J=16.8 Hz, 1H), 6.08-5.94 (m, 1H), 5.85 (d, J=10.0 Hz, 1H), 5.03 (q, J=7.2 Hz, 2H), 4.81-4.64 (m, 2H), 4.59-4.35 (m, 4H), 4.33-4.07 (m, 2H), 4.01-3.36 (m, 10H), 3.31-3.16 (m, 4H), 3.02 (s, 3H), 2.51-2.42 (m, 3H), 2.41-2.29 (m, 1H), 2.18 (dd, J=7.6, 13.0 Hz, 1H), 2.07-1.78 (m, 5H), 1.63-1.45 (m, 3H), 1.04 (d, J=6.4 Hz, 3H), 0.93-0.82 (m, 3H).

The mixture of atropisomers can be separated by SFC.

### (2S,4R)-1-((R)-2-(3-(2-((1-(2-(((S)-4-((S)-4-acryloyl-3-(cyanomethyl)piperazin-1-yl)-6-chloro-8-fluoro-7-(3-hydroxynaphthalen-1-yl)quinazolin-2-yl)oxy)ethyl)piperidin-4-yl)oxy)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

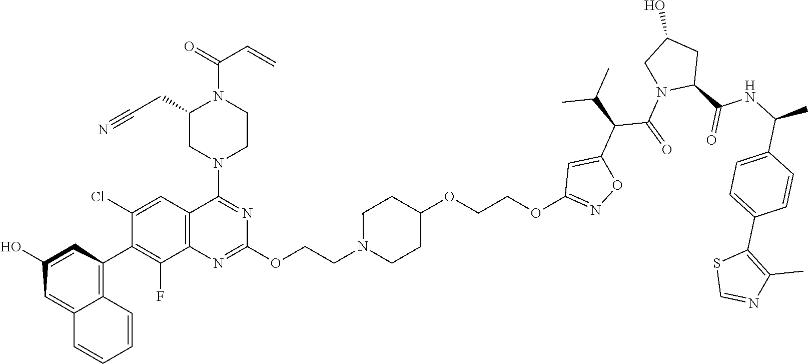

(white solid. Both absolute configuration of the naphthalene group and the isopropyl group are tentatively assigned). ¹H-NMR (400 MHz, CD₃OD) δ 8.87-8.84 (m, 1H), 8.50-8.40 (m, 1H), 8.07-8.06 (m, 1H), 7.76-7.74 (m, 1H), 7.45-7.19 (m, 8H), 7.04-7.03 (m, 1H), 6.90-6.80 (m, 1H), 6.33-6.29 (m, 1H), 6.02-5.97 (m, 1H), 5.85-5.83 (m, 1H), 5.03-5.01 (m, 2H), 4.87-4.28 (m, 9H), 3.85-3.61 (m, 10H), 3.30-2.70 (m, 7H), 2.46 (s, 3H), 2.41-2.40 (m, 2H), 2.03-1.86 (m, 5H), 1.53-1.49 (m, 3H), 1.02-1.00 (m, 3H), 0.88-0.86 (m, 3H).

### (2S,4R)-1-((R)-2-(3-(2-((1-(2-(((R)-4-((S)-4-acryloyl-3-(cyanomethyl)piperazin-1-yl)-6-chloro-8-fluoro-7-(3-hydroxynaphthalen-1-yl)quinazolin-2-yl)oxy)ethyl)piperidin-4-yl)oxy)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

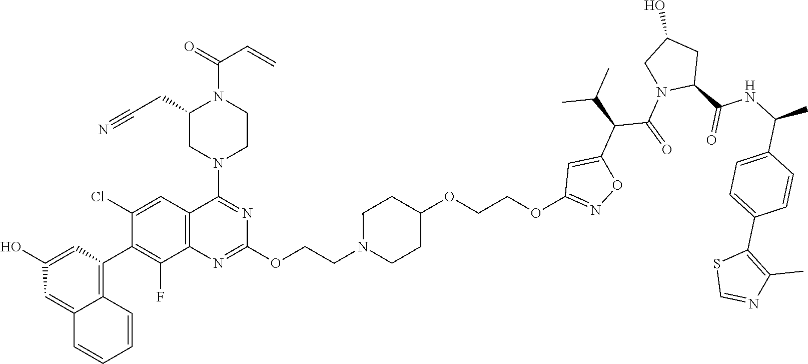

(white solid. Both absolute configuration of the naphthalene group and the isopropyl group are tentatively assigned). ¹H-NMR (400 MHz, CD₃OD) δ 8.87-8.84 (m, 1H), 8.50-8.40 (m, 1H), 8.07-8.06 (m, 1H), 7.76-7.74 (m, 1H), 7.45-7.19 (m, 8H), 7.04-7.03 (m, 1H), 6.90-6.80 (m, 1H), 6.33-6.29 (m, 1H), 6.02-5.97 (m, 1H), 5.85-5.83 (m, 1H), 5.03-5.01 (m, 2H), 4.87-4.28 (m, 9H), 3.85-3.61 (m, 10H), 3.30-2.70 (m, 7H), 2.46 (s, 3H), 2.41-2.40 (m, 2H), 2.03-1.86 (m, 5H), 1.53-1.49 (m, 3H), 1.02-1.00 (m, 3H), 0.88-0.86 (m, 3H).

The following compounds can be prepared in an analogous manner to 2S,4R)-1-[(2R)-2-[3-[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

### 1. (2S,4S)-1-((2S)-2-(3-(2-((1-(2-((4-((S)-4-acryloyl-3-(cyanomethyl)piperazin-1-yl)-6-chloro-8-fluoro-7-(3-hydroxynaphthalen-1-yl)quinazolin-2-yl)oxy)ethyl)piperidin-4-yl)oxy)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

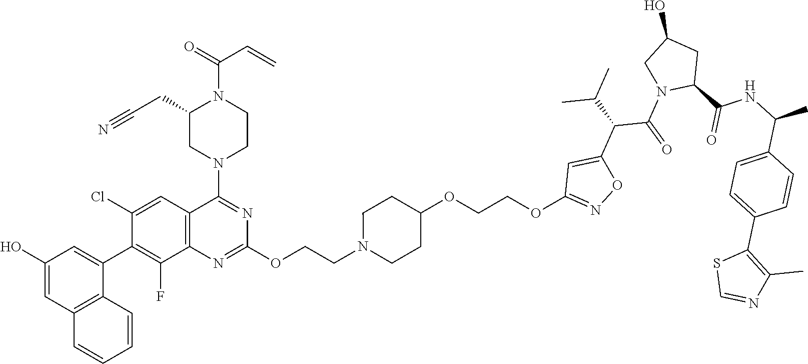 [SMILES: C=CC(=O)N1CCN(c2nc(OCCN3CCC(OCCOc4cc([C@@H](C(=O)N5C[C@@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(white solid, formic acid salt. The absolute configuration of the isopropyl group is tentatively assigned). LC/MS (ESI) m/z: 1169.4 [M+H]⁺. ¹H-NMR (400 MHz, CD₃OD) δ 8.87-8.82 (m, 1H), 8.50-8.40 (m, 1H), 8.07-8.06 (m, 1H), 7.76-7.74 (m, 1H), 7.45-7.19 (m, 8H), 7.04-7.03 (m, 1H), 6.90-6.80 (m, 1H), 6.33-6.29 (m, 1H), 6.02-5.97 (m, 1H), 5.85-5.83 (m, 1H), 5.09-4.96 (m, 1H), 4.87-4.28 (m, 9H), 3.85-3.61 (m, 8H), 3.30-2.70 (m, 8H), 2.46 (s, 3H), 2.41-2.40 (m, 2H), 2.03-1.86 (m, 5H), 1.53-1.49 (m, 3H), 1.02-0.77 (m, 6H).

### 2. (2S,4S)-1-((2R)-2-(3-(2-((1-(2-((4-((S)-4-acryloyl-3-(cyanomethyl)piperazin-1-yl)-6-chloro-8-fluoro-7-(3-hydroxynaphthalen-1-yl)quinazolin-2-yl)oxy)ethyl)piperidin-4-yl)oxy)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

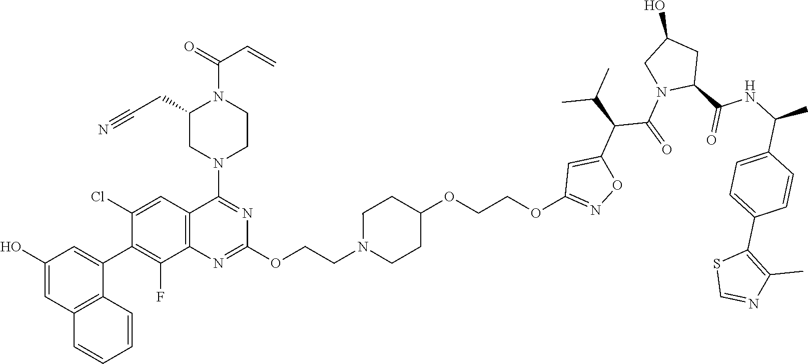 [SMILES: C=CC(=O)N1CCN(c2nc(OCCN3CCC(OCCOc4cc([C@H](C(=O)N5C[C@@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(white solid, formic acid salt. The absolute configuration of the isopropyl group is tentatively assigned). LC/MS (ESI) m/z: 1169.4 [M+H]⁺. ¹H-NMR (400 MHz, CD₃OD) δ 8.87-8.82 (m, 1H), 8.50-8.40 (m, 1H), 8.07-8.06 (m, 1H), 7.76-7.74 (m, 1H), 7.45-7.19 (m, 8H), 7.04-7.03 (m, 1H), 6.90-6.80 (m, 1H), 6.33-6.29 (m, 1H), 6.02-5.97 (m, 1H), 5.85-5.83 (m, 1H), 5.09-4.96 (m, 1H), 4.87-4.28 (m, 9H), 3.85-3.61 (m, 8H), 3.30-2.70 (m, 8H), 2.46 (s, 3H), 2.41-2.40 (m, 2H), 2.03-1.86 (m, 5H), 1.53-1.49 (m, 3H), 1.02-1.00 (m, 3H), 0.88-0.86 (m, 3H).

### 3. (2S,4R)-1-[(2R)-2-[3-[[1-[(2R)-2-[6-chloro-8-fluoro-7-(3-hydroxy-1-naphthyl)-4- (4-prop-2-enoylpiperazin-1-yl)quinazolin-2-yl]oxypropyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

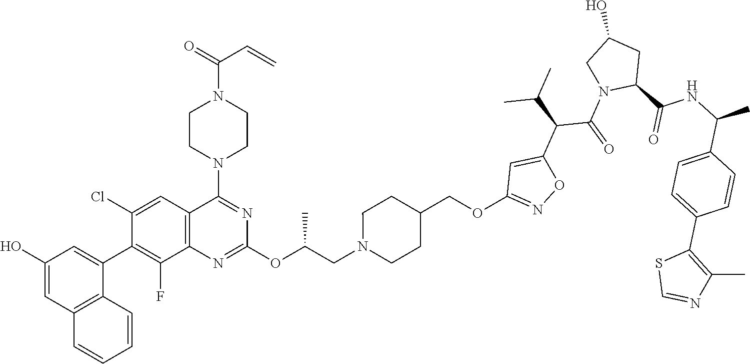 [SMILES: C=CC(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1]

(white solid). LC/MS (ESI) m/z: 1114.4 [M+H]⁺. ¹H-NMR (400 MHz, DMSO-d₆) δ 10.06-9.99 (m, 1H), 9.00-8.96 (m, 1H), 8.84-8.37 (m, 1H), 8.01 (s, 1H), 7.81 (br d, J=8.7 Hz, 1H), 7.49-7.31 (m, 6H), 7.29 (d, J=1.5 Hz, 1H), 7.24-7.17 (m, 2H), 7.08-7.05 (m, 1H), 6.83 (dd, J=10.4, 16.8 Hz, 1H), 6.18 (dd, J=2.1, 16.7 Hz, 1H), 6.04 (d, J=7.0 Hz, 1H), 5.78-5.72 (m, 1H), 5.47-5.35 (m, 1H), 5.09 (d, J=3.5 Hz, 1H), 4.95-4.85 (m, 1H), 4.39-4.32 (m, 1H), 4.27 (br d, J=1.3 Hz, 1H), 3.93 (br s, 6H), 3.85 (br d, J=2.1 Hz, 2H), 3.81-3.75 (m, 2H), 3.69 (br dd, J=4.5, 10.0 Hz, 1H), 3.62 (br d, J=10.0 Hz, 1H), 3.45-3.39 (m, 2H), 3.06-2.86 (m, 2H), 2.64-2.57 (m, 1H), 2.46-2.44 (m, 3H), 2.44-2.35 (m, 1H), 2.22 (br dd, J=3.0, 6.1 Hz, 1H), 2.09-1.94 (m, 3H), 1.81-1.73 (m, 1H), 1.71-1.54 (m, 3H), 1.44-1.36 (m, 3H), 1.34-1.30 (m, 3H), 0.98-0.92 (m, 3H), 0.81-0.74 (m, 3H).

### 4. (2S,4R)-1-[(2S)-2-[3-[[1-[(2R)-2-[6-chloro-8-fluoro-7-(3-hydroxy-1-naphthyl)-4- (4-prop-2-enoylpiperazin-1-yl)quinazolin-2-yl]oxypropyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

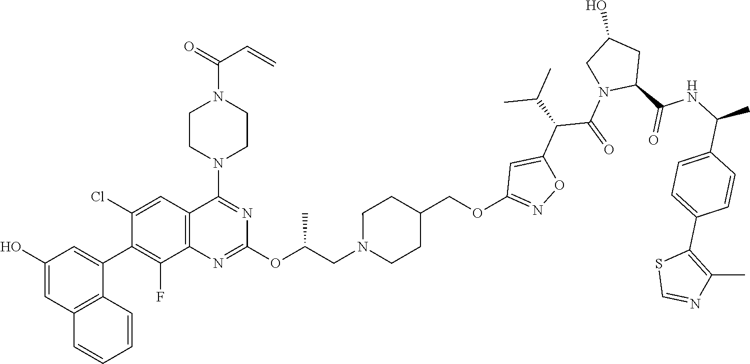 [SMILES: C=CC(=O)N1CCN(c2nc(O[C@H](C)CN3CCC(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1]

(white solid). LC/MS (ESI) m/z: 1114.4 [M+H]⁺. ¹H-NMR (400 MHz, DMSO-d₆) δ 10.08-9.95 (m, 1H), 9.05-8.86 (m, 1H), 8.30-8.22 (m, 1H), 8.05-7.93 (m, 1H), 7.80 (br d, J=7.7 Hz, 1H), 7.49-7.27 (m, 6H), 7.23-7.16 (m, 2H), 7.09-7.04 (m, 1H), 6.88-6.75 (m, 1H), 6.22-6.13 (m, 1H), 6.12-6.02 (m, 1H), 5.74 (br d, J=10.8 Hz, 1H), 5.46-5.34 (m, 1H), 5.12-4.93 (m, 1H), 4.88 (br t, J=6.9 Hz, 1H), 4.54-4.35 (m, 1H), 4.31-4.21 (m, 1H), 3.97-3.90 (m, 4H), 3.85 (br d, J=1.7 Hz, 3H), 3.80-3.66 (m, 3H), 3.57-3.41 (m, 2H), 3.10-2.80 (m, 3H), 2.65-2.58 (m, 1H), 2.43 (s, 3H), 2.40-2.36 (m, 1H), 2.28-2.19 (m, 1H), 2.09-1.91 (m, 3H), 1.82-1.72 (m, 1H), 1.66-1.48 (m, 3H), 1.40-1.26 (m, 6H), 1.16-1.03 (m, 2H), 0.95 (br d, J=6.7 Hz, 2H), 0.80 (br d, J=6.6 Hz, 3H), 0.72 (br d, J=3.8 Hz, 1H).

### Exemplary Synthesis of (2S,4R)-1-[(2R)-2-[3-[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of (2S,4R)-1-[(2R)-2-[3-[2-[[1-[2-[6-chloro-4-[(3S)-3-(cyanomethyl) piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

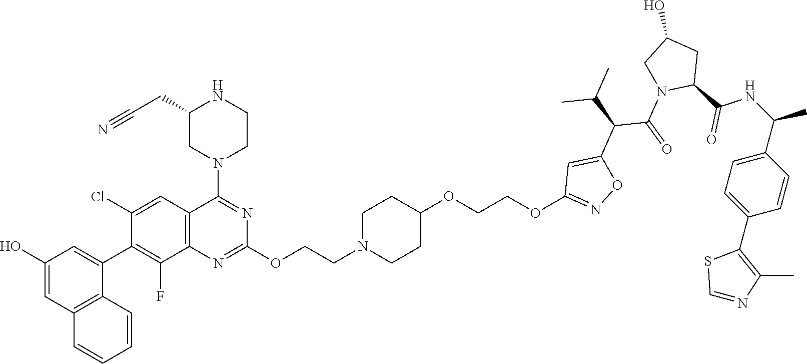 [SMILES: Cc1ncsc1-c1ccc([C@H](C)NC(=O)[C@@H]2C[C@@H](O)CN2C(=O)[C@@H](c2cc(OCCOC3CCN(CCOc4nc(N5CCN[C@@H](CC#N)C5)c5cc(Cl)c(-c6cc(O)cc7ccccc67)c(F)c5n4)CC3)no2)C(C)C)cc1]

To a solution of 2-[(2S)-4-[6-chloro-8-fluoro-7-(3-hydroxy-1-naphthyl)-2-(2-oxoethoxy) quinazolin-4-yl]piperazin-2-yl]acetonitrile (300 mg, 592.97 umol, 1 eq) and (2S,4R)-4-hydroxy-1-[(2R)-3-methyl-2-[3-[2-(4-piperidyloxy)ethoxy]isoxazol-5-yl]butanoyl]-N-[(1S)-1-[4-(4-methylthiazol-5-yl)phenyl]ethyl]pyrrolidine-2-carboxamide (389.62 mg, 622.62 umol, 1.05 eq) in dichloroethane (2 mL) and isopropanol (2 mL) were added 2-methylpyridine borane (317.12 mg, 2.96 mmol, 5 eq), acetic acid (3.56 mg, 59.30 umol, 3.39 uL, 0.1 eq), and NaOAc (486.41 mg, 5.93 mmol, 10 eq), and the reaction mixture was stirred at 40° C. for 8 hours. The suspension was filtered, and the filtrate was concentrated under reduced pressure. The resulting yellow solid was purified by prep-TLC on SiO₂ (CH₂Cl₂/CH₃OH 10/1) to give compound (2S,4R)-1-[(2R)-2-[3-[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (300 mg, 268.89 umol, 45% yield) as a white solid.

### Step 2: Preparation of (2S,4R)-1-[(2R)-2-[3-[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

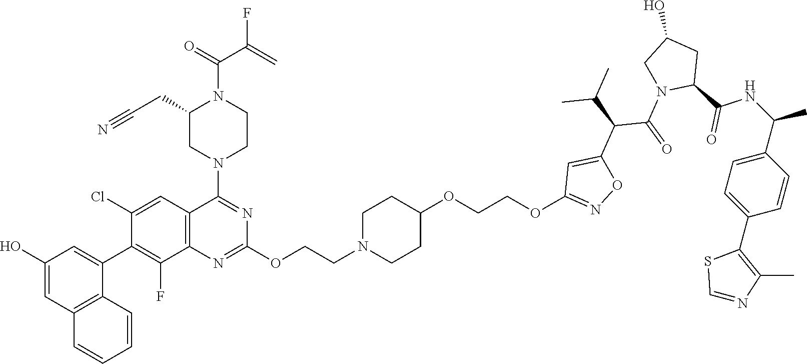 [SMILES: C=C(F)C(=O)N1CCN(c2nc(OCCN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

To a solution of (2S,4R)-1-[(2R)-2-[3-[2-[[1-[2-[6-chloro-4-[(3S)-3-(cyanomethyl) piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (280 mg, 250.96 umol, 1 eq) in DMF (2 mL) at 20° C. were added HATU (143.14 mg, 376.45 umol, 1.5 eq) and triethylamine (76.18 mg, 752.89 umol, 104.79 uL, 3 eq) followed by 2-fluoroprop-2-enoic acid (27.12 mg, 301.16 umol, 1.80 mL, 1.2 eq), and the reaction mixture was stirred at 20° C. for 6 hours. CH₃OH (1 mL) was added, and the resulting solution was concentrated under reduced pressure. The resulting yellow solid was purified by prep-HPLC (29%-59% CH₃CN in water (0.225% formic acid)) to give compound (2S,4R)-1-[(2R)-2-[3-[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (98.6 mg, 76.72 umol, 31% yield, formic acid salt) as a yellow solid. LC/MS (ESI) m/z: 1187.4 [M+H]⁺. ¹H-NMR (400 MHz, CD₃OD) δ 8.89-8.87 (m, 1H), 8.06-7.99 (m, 1H), 7.77-7.75 (m, 1H), 7.44-7.40 (m, 5H), 7.27-7.21 (m, 3H), 7.05 (d, J=2.4 Hz, 1H), 6.03-5.97 (m, 1H), 5.52-5.41 (m, 2H), 5.05-5.03 (m, 1H), 4.70-4.15 (m, 8H), 3.82-3.79 (m, 5H), 3.69-3.66 (m, 3H), 3.20-3.00 (m, 2H), 2.88-2.83 (m, 6H), 2.60 (s, 3H), 2.49-2.40 (m, 3H), 2.20-2.18 (m, 2H), 2.17-1.96 (m, 3H), 1.60-1.52 (m, 5H), 1.07-1.05 (m, 3H), 0.91-0.88 (m, 3H).

### Exemplary Synthesis of (2S,4R)-1-((2R)-2-(3-((1-((2R)-2-((6-chloro-8-fluoro-4-(4-(2-fluoroacryloyl)piperazin-1-yl)-7-(3-hydroxynaphthalen-1-yl)quinazolin-2-yl)oxy)propyl)piperidin-4-ylmethoxy)isoxazol-5-yl)-3-methylbutanoyl)-4-hydroxy-N—((S)-1-(4-(4-methylthiazol-5-yl)phenyl)ethyl)pyrrolidine-2-carboxamide

### Step 1: Preparation of (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide

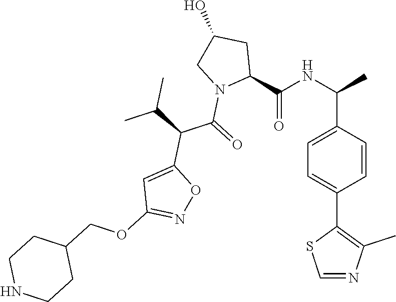 [SMILES: Cc1ncsc1-c1ccc([C@H](C)NC(=O)[C@@H]2C[C@@H](O)CN2C(=O)[C@@H](c2cc(OCC3CCNCC3)no2)C(C)C)cc1]

To a solution of tert-butyl 4-(((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidine-1-carboxylate (80 mg, 0.11 mmol, 1 eq) in CH₂Cl₂ (1 mL) was added HCl (4N in dioxane, 0.2 mL, 6.96 eq), and the reaction mixture was stirred at 25° C. for 0.5 hours. The reaction mixture was concentrated under reduced pressure to afford (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (72 mg, 0.11 mmol, 99% yield, HCl salt) as a yellow solid.

### Step 2: Preparation of tert-butyl 4-(6-chloro-8-fluoro-2-(((R)-1-(4-(((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidin-1-yl)propan-2-yl)oxy)-7-(3-hydroxynaphthalen-1-yl)quinazolin-4-yl)piperazine-1-carboxylate

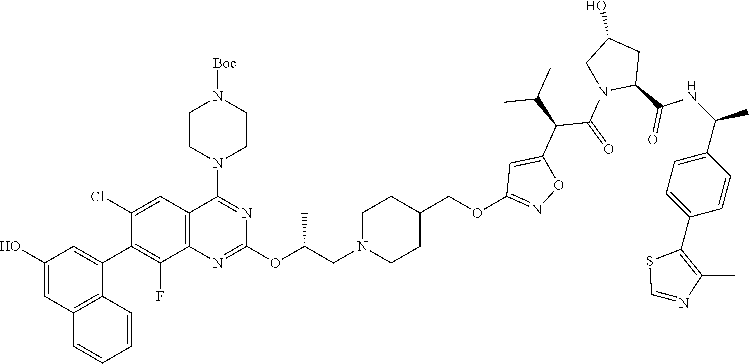 [SMILES: Cc1ncsc1-c1ccc([C@H](C)NC(=O)[C@@H]2C[C@@H](O)CN2C(=O)[C@@H](c2cc(OCC3CCN(C[C@@H](C)Oc4nc(N5CCN(C(=O)OC(C)(C)C)CC5)c5cc(Cl)c(-c6cc(O)cc7ccccc67)c(F)c5n4)CC3)no2)C(C)C)cc1]

To a solution of (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (125 mg, 0.2 mmol, 1 eq, HCl salt) in CH₃OH (3 mL) at 0° C. were added NaOAc (48.66 mg, 0.6 mmol, 3 eq) and 2-methylpyridine borane (106 mg, 0.99 mmol, 5 eq), and the resulting mixture was stirred at 0° C. for 0.5 hours. tert-Butyl 4-[6-chloro-8-fluoro-7-(3-hydroxy-1-naphthyl)-2-[(1R)-1-methyl-2-oxo-ethoxy]quinazolin-4-yl]piperazine-1-carboxylate (115 mg, 0.2 mmol, 1 eq) and acetic acid (1.19 mg, 0.02 mmol, 0.001 mL, 0.1 eq) were then added, and the reaction mixture was stirred at 40° C. for 11.5 h. The mixture was concentrated under reduced pressure, and the resulting residue was purified by prep-TLC on SiO₂ (CH₂Cl₂:CH₃OH=10:1) to afford tert-butyl 4-(6-chloro-8-fluoro-2-(((R)-1-(4-(((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidin-1-yl)propan-2-yl)oxy)-7-(3-hydroxynaphthalen-1-yl)quinazolin-4-yl)piperazine-1-carboxylate (60 mg, 0.05 mmol, 25% yield) as a yellow solid. LC/MS (ESI) m/z: 581.3 [M/2+H]⁺.

### Step 3: Preparation of (2S,4R)-1-((2R)-2-(3-((1-((2R)-2-((6-chloro-8-fluoro-7-(3-hydroxynaphthalen-1-yl)-4-(piperazin-1-yl)quinazolin-2-yl)oxy)propyl)piperidin-4-ylmethoxy)isoxazol-5-yl)-3-methylbutanoyl)-4-hydroxy-N—((S)-1-(4-(4-methylthiazol-5-yl)phenyl)ethyl)pyrrolidine-2-carboxamide

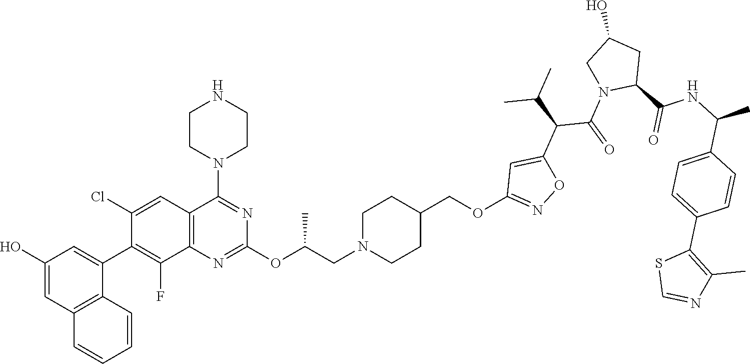 [SMILES: Cc1ncsc1-c1ccc([C@H](C)NC(=O)[C@@H]2C[C@@H](O)CN2C(=O)[C@@H](c2cc(OCC3CCN(C[C@@H](C)Oc4nc(N5CCNCC5)c5cc(Cl)c(-c6cc(O)cc7ccccc67)c(F)c5n4)CC3)no2)C(C)C)cc1]

To a solution of tert-butyl 4-(6-chloro-8-fluoro-2-(((R)-1-(4-(((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidin-1-yl)propan-2-yl)oxy)-7-(3-hydroxynaphthalen-1-yl)quinazolin-4-yl)piperazine-1-carboxylate (60 mg, 0.05 mmol, 1 eq) in CH₂Cl₂ (3 mL) was added HCl (4N in dioxane, 2.25 mL, 174.12 eq), and the reaction mixture was stirred at 20° C. for 1 hour The mixture was concentrated under reduced pressure to give (2S,4R)-1-((2R)-2-(3-((1-((2R)-2-((6-chloro-8-fluoro-7-(3-hydroxynaphthalen-1-yl)-4-(piperazin-1-yl)quinazolin-2-yl)oxy)propyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide (61 mg, HCl salt) as a colorless oil.

### Step 4: Preparation of (2S,4R)-1-((2R)-2-(3-((1-((2R)-2-((6-chloro-8-fluoro-4-(4-(2-fluoroacryloyl)piperazin-1-yl)-7-(3-hydroxynaphthalen-1-yl)quinazolin-2-yl)oxy)propyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide

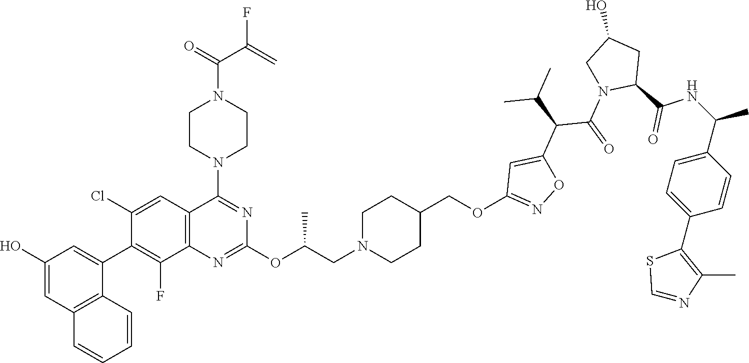 [SMILES: C=C(F)C(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1]

A mixture of (2S,4R)-1-((2R)-2-(3-((1-((2R)-2-((6-chloro-8-fluoro-7-(3-hydroxynaphthalen-1-yl)-4-(piperazin-1-yl)quinazolin-2-yl)oxy)propyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide (61 mg, 0.05 mmol, 1 eq, HCl salt), 2-fluoroprop-2-enoic acid (4.59 mg, 0.05 mmol, 1 eq), triethylamine (10 mg, 0.1 mmol, 0.01 mL, 2 eq), and HATU (29.06 mg, 0.08 mmol, 1.5 eq) in DMF (2 mL) was stirred at 20° C. for 0.5 hours under nitrogen atmosphere. The mixture was filtered and concentrated under reduced pressure. The resulting residue was purified by semi-preparative reverse phase HPLC (29-59% CH₃CN in water (0.225% formic acid)) followed by prep-TLC (CH₂Cl₂:CH₃OH=10:1) to afford (2S,4R)-1-((2R)-2-(3-((1-((2R)-2-((6-chloro-8-fluoro-4-(4-(2-fluoroacryloyl)piperazin-1-yl)-7-(3-hydroxynaphthalen-1-yl)quinazolin-2-yl)oxy)propyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide (3.0 mg, 0.002 mmol, 5% yield) as a white solid. LC/MS (ESI) m/z: 566.8 [M/2+1]⁺. ¹H-NMR (400 MHz, DMSO-d₆) δ 10.02 (s, 1H), 8.98 (s, 1H), 8.40 (d, J=7.8 Hz, 1H), 8.01 (s, 1H), 7.81 (d, J=8.0 Hz, 1H), 7.46-7.40 (m, 3H), 7.38-7.33 (m, 2H), 7.29 (s, 1H), 7.24-7.15 (m, 2H), 7.10-7.03 (m, 1H), 6.04 (d, J=6.5 Hz, 1H), 5.46-5.32 (m, 2H), 5.09 (d, J=3.8 Hz, 1H), 4.90 (t, J=7.2 Hz, 1H), 4.43-4.22 (m, 2H), 3.95 (s, 5H), 3.80 (s, 2H), 3.72-3.67 (m, 1H), 3.64-3.54 (m, 1H), 3.46-3.38 (m, 2H), 3.31 (s, 6H), 3.19-3.09 (m, 1H), 2.94 (s, 1H), 2.46-2.44 (m, 3H), 2.27-2.14 (m, 2H), 2.08-1.93 (m, 3H), 1.81-1.72 (m, 1H), 1.71-1.55 (m, 3H), 1.45-1.31 (m, 5H), 1.24-1.21 (m, 1H), 0.94 (d, J=6.5 Hz, 3H), 0.83-0.75 (m, 3H).

The following compounds were prepared in an analogous manner to (2S,4R)-1-((2R)-2-(3-((1-((2R)-2-((6-chloro-8-fluoro-4-(4-(2-fluoroacryloyl)piperazin-1-yl)-7-(3-hydroxynaphthalen-1-yl)quinazolin-2-yl)oxy)propyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide

### 1. (2S,4R)-1-((2S)-2-(3-((1-((2R)-2-((6-chloro-8-fluoro-4-(4-(2-fluoroacryloyl)piperazin-1-yl)-7-(3-hydroxynaphthalen-1-yl)quinazolin-2-yl)oxy)propyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide

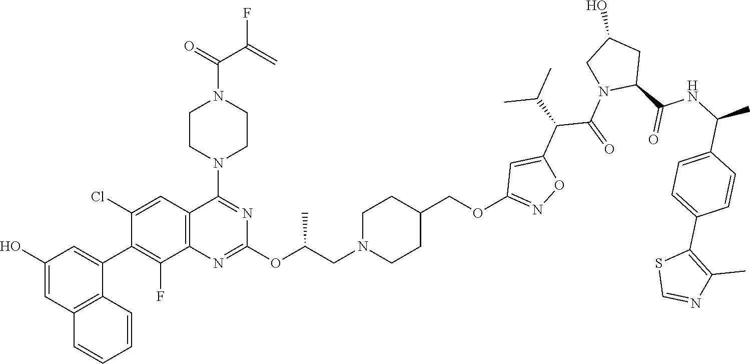 [SMILES: C=C(F)C(=O)N1CCN(c2nc(O[C@H](C)CN3CCC(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1]

(white solid). LC/MS (ESI) m/z: 566.8 [M/2+1]⁺. ¹H-NMR (400 MHz, DMSO-d₆) δ 10.03 (s, 1H), 9.02-8.87 (m, 1H), 8.27 (d, J=7.5 Hz, 1H), 8.00 (s, 1H), 7.80 (d, J=7.7 Hz, 1H), 7.49-7.36 (m, 4H), 7.29 (d, J=7.5 Hz, 2H), 7.24-7.16 (m, 2H), 7.08-7.03 (m, 1H), 6.13-6.01 (m, 1H), 5.42-5.33 (m, 2H), 5.23-5.06 (m, 1H), 5.02-4.83 (m, 1H), 4.40 (t, J=7.6 Hz, 1H), 4.25 (s, 1H), 3.97-3.86 (m, 5H), 3.79 (s, 3H), 3.73 (d, J=9.2 Hz, 1H), 3.56-3.39 (m, 6H), 3.03-2.84 (m, 2H), 2.66-2.57 (m, 1H), 2.43 (d, J=1.7 Hz, 3H), 2.41-2.36 (m, 1H), 2.23 (dd, J=6.9, 15.3 Hz, 1H), 2.06-1.89 (m, 3H), 1.81-1.72 (m, 1H), 1.57 (s, 3H), 1.44 (d, J=6.8 Hz, 1H), 1.36-1.29 (m, 5H), 1.11 (s, 1H), 0.95 (d, J=6.6 Hz, 2H), 0.80 (d, J=6.8 Hz, 3H), 0.72 (d, J=6.4 Hz, 1H).

### 2. (2S,4R)-1-[(2S)-2-[3-[2-[[1-[2-[6-chloro-4-[(3S)-3-(cyanomethyl)-4-prop-2- enoyl-piperazin-1-yl]-8-fluoro-7-(3-hydroxy-1-naphthyl)quinazolin-2-yl]oxyethyl]-4-piperidyl]oxy]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

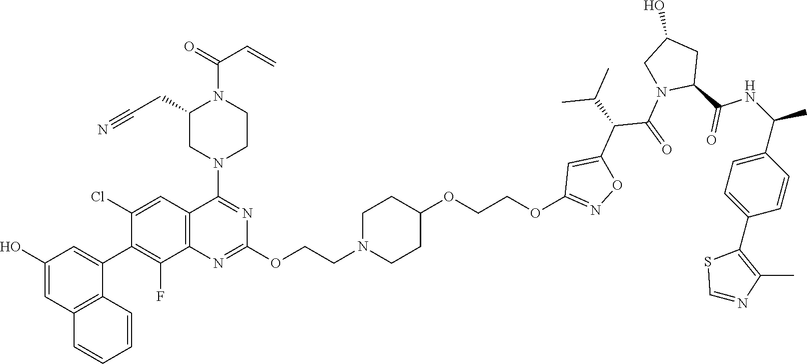 [SMILES: C=CC(=O)N1CCN(c2nc(OCCN3CCC(OCCOc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(formic acid salt, white solid). LC/MS (ESI) m/z: 585.4 [M/2+1]⁺. ¹H-NMR (400 MHz, DMSO-d₆) δ 9.03-8.95 (m, 1H), 8.92 (d, J=7.8 Hz, 1H), 8.30 (d, J=7.7 Hz, 1H), 8.27 (s, 1H), 8.14-8.08 (m, 1H), 7.82 (d, J=8.3 Hz, 1H), 7.50-7.44 (m, 1H), 7.44-7.39 (m, 2H), 7.33-7.29 (m, 2H), 7.25-7.14 (m, 2H), 7.08 (d, J=2.3 Hz, 1H), 7.01-6.76 (m, 1H), 6.22 (dd, J=2.0, 16.6 Hz, 1H), 6.17-6.06 (m, 1H), 5.87-5.69 (m, 1H), 5.06-4.96 (m, 1H), 4.93-4.85 (m, 1H), 4.54-4.39 (m, 3H), 4.38-4.31 (m, 1H), 4.29-4.18 (m, 5H), 4.29-3.78 (m, 1H), 3.77-3.64 (m, 4H), 3.57-3.53 (m, 1H), 3.49-3.45 (m, 2H), 3.41 (br s, 2H), 3.30 (br d, J=6.4 Hz, 2H), 3.14-2.99 (m, 2H), 2.83-2.74 (m, 2H), 2.71-2.65 (m, 2H), 2.45 (s, 3H), 2.30-2.22 (m, 1H), 2.18-2.09 (m, 2H), 2.08-2.00 (m, 1H), 1.88-1.74 (m, 3H), 1.48-1.38 (m, 2H), 1.35 (d, J=7.0 Hz, 3H), 0.96 (d, J=6.6 Hz, 2H), 0.83 (d, J=6.6 Hz, 3H), 0.75 (d, J=6.6 Hz, 1H).

### Exemplary Synthesis of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of tert-butyl (2S)-4-[6-chloro-2-(2,2-dimethoxyethoxy)-8-fluoro-7-(3-hydroxy-1-naphthyl)quinazolin-4-yl]-2-(cyanomethyl)piperazine-1-carboxylate

### Step 2: Preparation of 2-[(2S)-4-[6-chloro-8-fluoro-7-(3-hydroxy-1-naphthyl)-2-(2-oxoethoxy)quinazolin-4-yl]piperazin-2-yl]acetonitrile

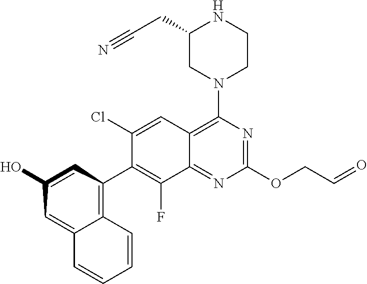

To tert-butyl (2S)-4-[6-chloro-2-(2,2-dimethoxyethoxy)-8-fluoro-7-(3-hydroxy-1-naphthyl)quinazolin-4-yl]-2-(cyanomethyl)piperazine-1-carboxylate (8.0 g, 12.27 mmol, 1.0 eq) in CH₃CN (22 mL) was added HCl (21.6 mL, 223.6 mmol, 37% in water, 18.23 eq), and the reaction mixture was stirred at 25° C. for 20 minutes. The mixture was poured into saturated aqueous NaHCO₃ (500 mL), and the resulting precipitate was filtered. This material as dissolved in THF/EtOAc (1:2, 300 mL), and the organic mixture was dried over Na₂SO₄, filtered, and dried under reduced pressure to afford 2-[(2S)-4-[6-chloro-8-fluoro-7-(3-hydroxy-1-naphthyl)-2-(2-oxoethoxy)quinazolin-4-yl]piperazin-2-yl]acetonitrile (6.21 g) as a yellow solid. LC/MS (ESI) m/z: 506.1 [M+H]⁺.

### Step 3: Preparation of (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

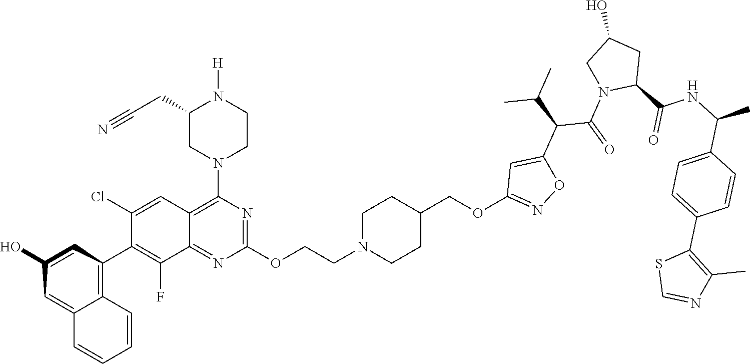

To 2-[(2S)-4-[6-chloro-8-fluoro-7-(3-hydroxy-1-naphthyl)-2-(2-oxoethoxy)quinazolin-4-yl]piperazin-2-yl]acetonitrile (2.33 g, 4.61 mmol, 1.0 eq) in isopropanol/CH₂Cl₂ (1:1; 100 mL) were added (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (2.47 g, 4.14 mmol, 0.9 eq), HOAc (1.11 g, 18.42 mmol, 1.05 mL, 4.0 eq), and 2-picoline borane (2.46 g, 23.03 mmol, 5.0 eq), and the reaction mixture was stirred at 25° C. for 1 hour. The reaction mixture was concentrated under reduced pressure, and the crude product was purified by prep-HPLC (40-63% CH₃CN in water (10 mM NH₄HCO₃)) to afford (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (2.85 g, 2.53 mmol, 55% yield) as a white solid. LC/MS (ESI) m/z: 1085.3 [M+H]⁺.

### Step 4: Preparation of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

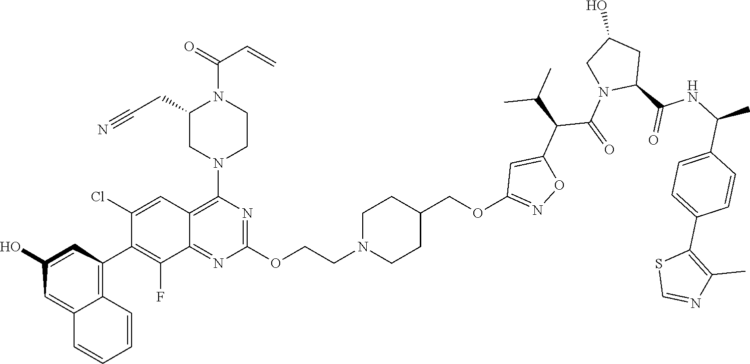

To a solution of (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (2.4 g, 2.21 mmol, 1 eq) in CH₂Cl₂ (40 mL) at −78° C. were added 2,6-lutidine (947.5 mg, 8.84 mmol, 4 eq) and prop-2-enoyl chloride (220.1 mg, 2.43 mmol, 1.1 eq) dropwise, and the reaction mixture was stirred at −78° C. for 30 minutes. Water (2.0 mL) was then added, and the resulting mixture was concentrated in vacuo. The resulting residue was purified by prep-HPLC (10-48% CH₃CN in water (0.225% formic acid)) to afford (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (1.30 g, 1.05 mmol, 48% yield, formic acid salt) as a white solid. LC/MS (ESI) m/z: 1139.5 [M+H]⁺. ¹H-NMR (400 MHz, CD₃OD) δ 8.90-8.85 (m, 1H), 8.47 (s, 1H), 8.09 (s, 1H), 7.75 (d, J=8.4 Hz, 1H), 7.47-7.35 (m, 5H), 7.27 (d, J=2.0 Hz, 1H), 7.24-7.14 (m, 2H), 7.03 (d, J=2.4 Hz, 1H), 6.31 (d, J=16.4 Hz, 1H), 5.99 (s, 1H), 5.89-5.78 (m, 1H), 5.14-4.97 (m, 2H), 4.81-4.72 (m, 3H), 4.56-4.46 (m, 3H), 4.46-4.37 (m, 1H), 4.22-4.13 (m, 1H), 4.11-4.03 (m, 2H), 3.97-3.80 (m, 3H), 3.69-3.65 (m, 2H), 3.42-3.33 (m, 2H), 3.07-2.97 (m, 2H), 2.92-2.72 (m, 2H), 2.48 (s, 3H), 2.43-2.28 (m, 1H), 2.23-2.14 (m, 1H), 2.12-1.91 (m, 4H), 1.66-1.57 (m, 2H), 1.52 (d, J=7.2 Hz, 3H), 1.05 (d, J=6.4 Hz, 3H), 0.95-0.83 (m, 3H).

### Exemplary Synthesis of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

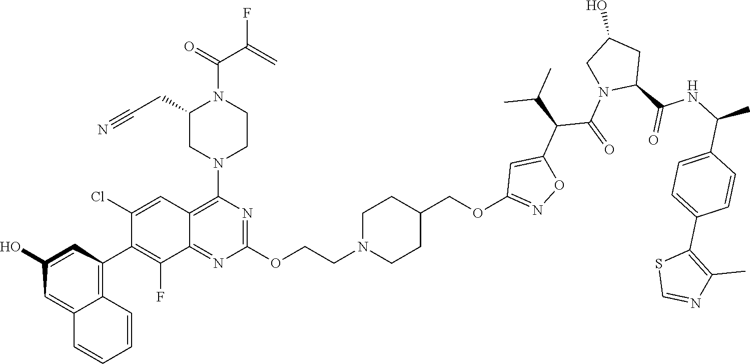

To a solution of 2-fluoroprop-2-enoic acid (167.97 mg, 1.87 mmol, 1.5 eq) in DMF (15 mL) were added diisopropylethylamine (482 mg, 3.73 mmol, 0.650 mL, 3 eq), HATU (1.42 g, 3.73 mmol, 3 eq), and (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2yl]oxyethyl]-4-piperidyl]methoxy]isoxazol-5-yl]-3-methyl-butanoyl]-4-hydroxy-N[(1S)-1-[4-(4-methylthiazol-5-yl)phenyl]ethyl]pyrrolidine-2-carboxamide (1.35 g, 1.24 mmol, 1 eq), and the reaction mixture was stirred at 25° C. for 2 hours. Saturated aqueous K₂CO₃ (10 mL) was then added, and the resulting mixture was stirred at 25° C. for 30 minutes. The mixture was extracted with CH₂Cl₂ (3×50 mL), and the combined extracts were dried over anhydrous Na₂SO₄, filtered, and concentrated. The resulting residue was purified by prep-HPLC (15-45% CH₃CN in water (0.225% formic acid)) to afford (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5yl)phenyl]ethyl]pyrrolidine-2-carboxamide (795.6 mg, 0.660 mmol, 53% yield, formic acid salt) as a white solid. LC/MS (ESI) m/z: 1157.4 [M+H]⁺. ¹H-NMR (400 MHz, DMSO-d₆) δ 8.98 (s, 1H), 8.41 (d, J=7.2 Hz, 1H), 8.15 (s, 1H), 8.11 (s, 1H), 7.81 (d, J=8.4 Hz, 1H), 7.47-7.41 (m, 4H), 7.39-7.33 (m, 2H), 7.29 (d, J=2.4 Hz, 1H), 7.22 (d, J=3.6 Hz, 2H), 7.06 (d, J=2.4 Hz, 1H), 6.06 (s, 1H), 5.49-5.09 (m, 3H), 4.90 (t, J=7.2 Hz, 2H), 4.74-4.17 (m, 8H), 3.98 (d, J=5.6 Hz, 4H), 3.75-3.55 (m, 6H), 3.11-2.86 (m, 3H), 2.72 (s, 2H), 2.45 (s, 3H), 2.33 (s, 1H), 2.03 (s, 2H), 1.68 (d, J=11.6 Hz, 3H), 1.40-1.25 (m, 5H), 0.94 (d, J=6.4 Hz, 3H), 0.78 (d, J=6.8 Hz, 3H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

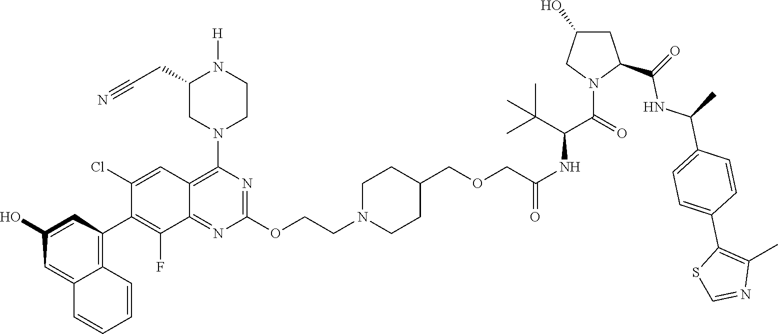

To a mixture of 2-[(2S)-4-[6-chloro-8-fluoro-7-(3-hydroxy-1-naphthyl)-2-(2-oxoethoxy)quinazolin-4-yl]piperazin-2-yl]acetonitrile (968 mg, 1.91 mmol, 1 eq) and (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (1.03 g, 1.72 mmol, 0.9 eq) in isopropanol (30 mL) and CH₂Cl₂ (30 mL) were added HOAc (460 mg, 7.65 mmol, 0.438 mL, 4 eq) and borane; 2-methylpyridine (1.02 g, 9.57 mmol, 5 eq), and the reaction mixture was stirred at 25° C. for 1 hour. The mixture was concentrated under reduced pressure, and the resulting crude product was purified by prep-HPLC (CH₃CN in water (0.05% NH₄OH+10 mM NH₄HCO₃)) to afford (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (935 mg, 0.79 mmol, 41% yield) as a white solid. LC/MS (ESI) m/z: 545.3 [M/2+H]⁺.

### Step 2: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

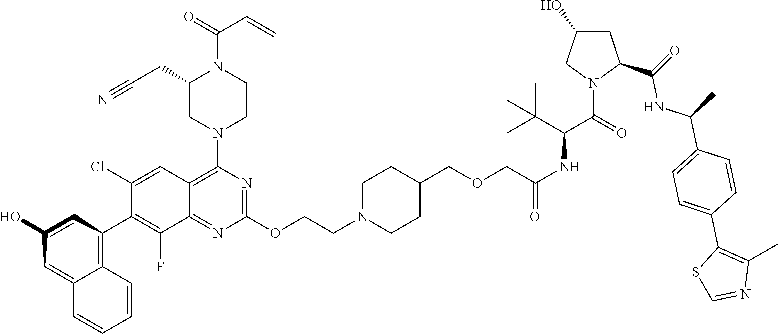

To a mixture of (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (2.66 g, 2.20 mmol, 90% purity, 1 eq) and 2,6-dimethylpyridine (1.18 g, 11.0 mmol, 1.28 mL, 5 eq) in CH₂Cl₂ (54 mL) at −78° C. was added a solution of prop-2-enoyl chloride (209 mg, 2.31 mmol, 0.188 mL, 1.05 eq) in CH₂Cl₂ (2 mL) slowly, and the resulting suspension was stirred at −78° C. for 30 minutes. Water (10 mL) was then added, and the resulting mixture was warmed to room temperature. The mixture was concentrated, and the resulting crude product was purified by prep-HPLC (CH₃CN in water (0.225% formic acid)) to afford (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (1.80 g, 1.56 mmol, 71% yield, formic acid salt) as a white solid. LC/MS (ESI) m/z: 1143.7 [M+H]⁺. ¹H-NMR (400 MHz, DMSO-d₆) δ 8.98 (s, 1H), 8.45 (d, J=7.6 Hz, 1H), 8.11 (s, 1H), 7.81 (d, J=8.4 Hz, 1H), 7.47-7.39 (m, 3H), 7.38-7.32 (m, 2H), 7.32-7.25 (m, 2H), 7.22 (d, J=3.6 Hz, 2H), 7.07 (d, J=2.4 Hz, 1H), 6.98-6.79 (m, 1H), 6.21 (d, J=16.4 Hz, 1H), 5.79 (d, J=10.8 Hz, 1H), 4.99 (s, 1H), 4.91-4.83 (m, 1H), 4.53 (d, J=9.6 Hz, 1H), 4.50-4.42 (m, 3H), 4.41-4.20 (m, 4H), 4.07 (s, 1H), 3.90 (s, 2H), 3.79-3.55 (m, 8H), 3.05-2.91 (m, 4H), 2.72 (s, 2H), 2.45 (s, 3H), 2.09-1.99 (m, 3H), 1.79-1.71 (m, 1H), 1.68-1.53 (m, 3H), 1.34 (d, J=6.8 Hz, 3H), 1.25-1.14 (m, 2H), 0.92 (s, 9H).

### Exemplary Synthesis of (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

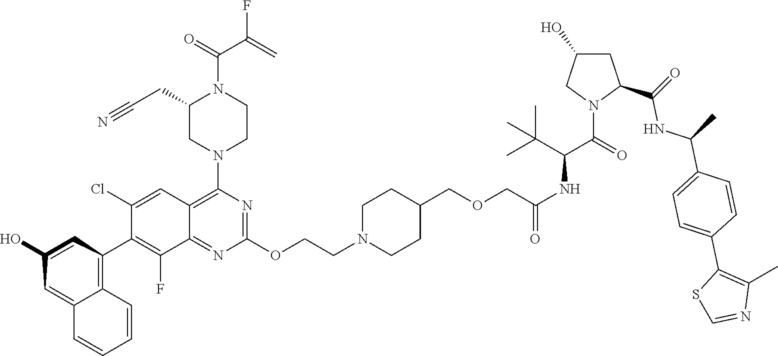

To (2S,4R)-1-[(2S)-2-[[2-[[1-[2-[6-chloro-4-[(3S)-3-(cyanomethyl)piperazin-1-yl]-8-fluoro- 7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (1.40 g, 1.28 mmol, 1 eq) in DMF (17 mL) were added diisopropylethylamine (498 mg, 3.85 mmol, 0.671 mL, 3 eq), HATU (1.47 g, 3.85 mmol, 3 eq), and 2-fluoroprop-2-enoic acid (139 mg, 1.54 mmol, 1.2 eq), and the reaction mixture was stirred at 25° C. for 2 hours. Saturated aqueous K₂CO₃ (30 mL) was then added, and the resulting mixture was stirred for 30 minutes. The mixture was extracted with 1:1 EtOAc/THF (3×30 mL), and the combined organic layers were dried over anhydrous Na₂SO₄, filtered, and concentrated. The resulting crude product was purified by prep-HPLC (CH₃CN in water (0.225% formic acid)) to afford (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (931 mg, 0.74 mmol, 58% yield, formic acid salt) as a white solid. LC/MS (ESI) m/z: 1161.6 [M+H]⁺. ¹H-NMR (400 MHz, DMSO-d₆) δ 10.05 (s, 1H), 8.98 (s, 1H), 8.45 (d, J=7.6 Hz, 1H), 8.11 (s, 1H), 7.81 (d, J=8.4 Hz, 1H), 7.47-7.39 (m, 3H), 7.37-7.32 (m, 2H), 7.31-7.26 (m, 2H), 7.22 (d, J=4.0 Hz, 2H), 7.07 (d, J=2.4 Hz, 1H), 5.42 (dd, J=18.0, 4.0 Hz, 2H), 5.26-5.09 (m, 1H), 4.96-4.76 (m, 2H), 4.53 (d, J=9.6 Hz, 1H), 4.50-4.40 (m, 3H), 4.40-4.25 (m, 3H), 3.91 (s, 2H), 3.64-3.48 (m, 2H), 3.33-3.31 (m, 6H), 3.08 (d, J=6.0 Hz, 1H), 3.06-2.95 (m, 3H), 2.78-2.71 (m, 2H), 2.45 (s, 3H), 2.13-1.99 (m, 3H), 1.79-1.70 (m, 1H), 1.70-1.52 (m, 3H), 1.34 (d, J=6.8 Hz, 3H), 1.27-1.14 (m, 2H), 0.92 (s, 9H).

### Exemplary Synthesis of (2S,4R)-1-[(2S)-2-[[2-[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[2-[[1-[2-[6-Chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide.

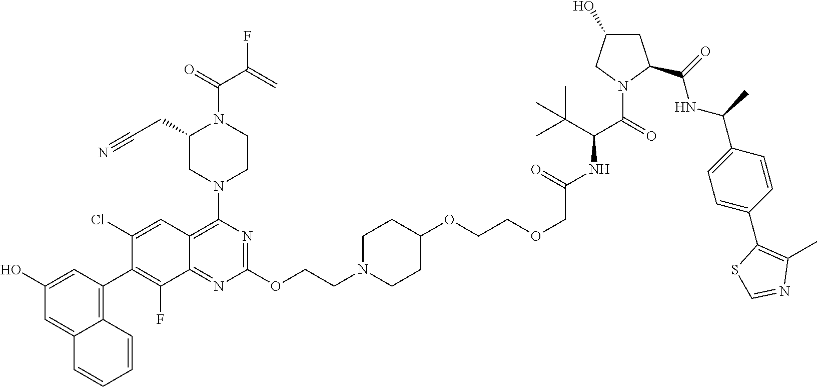 [SMILES: C=C(F)C(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(formic acid salt, white solid). LC/MS (ESI) m/z: 1191.9 [M+H]⁺. ¹H-NMR (400 MHz, CD₃OD) δ 8.87 (s, 1H), 8.46 (brs, 1H), 8.09 (s, 1H), 7.76 (d, J=7.6 Hz, 1H), 7.59-7.30 (m, 6H), 7.28 (s, 1H), 7.25-7.15 (m, 2H), 7.05 (s, 1H), 5.44-5.32 (m, 2H), 5.05-4.93 (m, 2H), 4.81-4.70 (m, 3H), 4.60-4.40 (m, 4H), 4.35-4.10 (m, 1H), 4.06 (s, 2H), 3.90-3.60 (m, 10H), 3.55-3.35 (m, 4H), 3.20-3.00 (m, 4H), 2.46 (d, J=2.4 Hz, 3H), 2.30-2.15 (m, 1H), 2.10-1.85 (m, 5H), 1.57, 1.48 (d, J=6.8 Hz, 3H), 1.04 (s, 9H).

### Exemplary Synthesis of (2S,4R)-1-[(2S)-2-[[2-[[1-[2-[6-chloro-4-[(3S)-3-(cyanomethyl)-4-[(E)-4-(dimethylamino)but-2-enoyl]piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

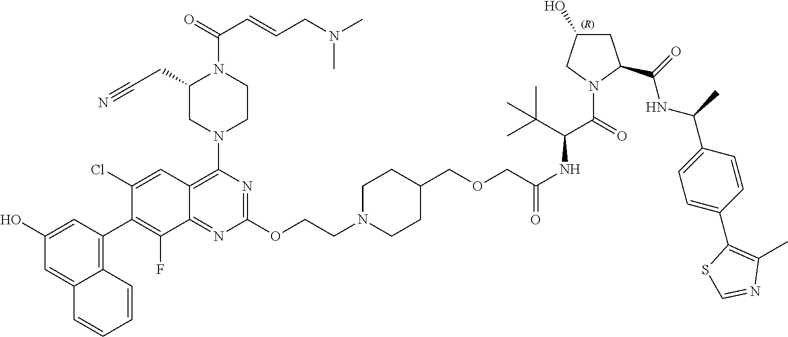

To a (E)-4-(dimethylamino)but-2-enoic acid (36.5 mg, 0.220 mmol, 2 eq, HCl salt) and diisopropylethylamine (142 mg, 1.10 mmol, 10 eq) in CH₂Cl₂ (12 mL) was added (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (120 mg, 0.110 mmol, 1 eq) followed by HATU (63 mg, 0.165 mmol, 1.5 eq), and the reaction mixture was stirred at 25° C. for 1 hour. The mixture was concentrated, and the resulting residue was purified by prep-HPLC (10-60% CH₃CN in water (0.225% formic acid)) to afford (2S,4R)-1-[(2S)-2-[[2-[[1-[2-[6-chloro-4-[(3S)-3-(cyanomethyl)-4-[(E)-4-(dimethylamino)but-2-enoyl]piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (38.9 mg, 30.91 umol, 28% yield, formic acid salt) as a white solid. LC/MS (ESI) m/z: 1200.7 [M+H]⁺. ¹H-NMR (400 MHz, CDCl₃) δ 8.67 (s, 1H), 8.42 (s, 1H), 7.90 (dd, J=8.4, 3.6 Hz, 1H), 7.81-7.73 (m, 2H), 7.59-7.49 (m, 2H), 7.43-7.31 (m, 6H), 7.30-7.25 (m, 2H), 7.27-7.15 (m, 2H), 6.30-6.21 (m, 1H), 5.14-5.00 (m, 1H), 4.79-4.65 (m, 3H), 4.62-4.46 (m, 2H), 4.40-4.19 (m, 2H), 4.10-3.96 (m, 2H), 3.92-3.84 (m, 1H), 3.64-3.57 (m, 1H), 3.49-3.05 (m, 15H), 2.68-2.55 (m, 2H), 2.51 (d, J=2.4 Hz, 3H), 2.45-2.39 (m, 2H), 2.34 (s, 6H), 1.89-1.64 (m, 4H), 1.60-1.49 (m, 2H), 1.46 (dd, J=8.4, 7.2 Hz, 3H), 1.04 (s, 9H).

### Exemplary Synthesis of (2S,4R)-1-[(2S)-2-[[2-[[1-[2-[6-chloro-4-[(3S)-3-(cyanomethyl)-4-[(E)-4-morpholinobut-2-enoyl]piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 4-(2,2-dimethoxyethyl)morpholine

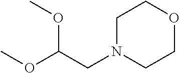

To a solution of 2,2-dimethoxyacetaldehyde (15 g, 86.45 mmol, 60% wt. % in water, 1 eq) and morpholine (11.30 g, 129.68 mmol, 11.41 mL, 1.5 eq) in CH₃OH (120 mL) was added AcOH (7.79 g, 129.68 mmol, 7.42 mL, 1.5 eq), and the resulting mixture was stirred at 20° C. for 3 hours. NaBH₃CN (7.06 g, 112.39 mmol, 1.3 eq) was then added, and the reaction mixture was stirred at 20° C. for 16 hours. The reaction mixture was quenched by addition of saturated aqueous Na₂CO₃ (100 mL) and stirred for 0.5 hours. The mixture was filtered, and the filter cake was washed with EtOAc (3×80 mL). The filtrate was washed with brine (30 mL), dried over anhydrous sodium sulfate, filtered, and concentrated. The resulting residue was purified by flash chromatography on SiO₂ (gradient: 0-20% EtOAc in petroleum ether) to afford 4-(2,2-dimethoxyethyl)morpholine (10.03 g, 57.24 mmol, 66% yield) as a colorless oil. ¹H-NMR (400 MHz, CDCl₃) δ 4.53 (t, J=5.2 Hz, 1H), 3.75-3.69 (m, 4H), 3.37 (s, 6H), 2.56-2.49 (m, 6H).

### Step 2: Preparation of (1-hydroxy-2-morpholino-ethyl)sulfonyloxysodium

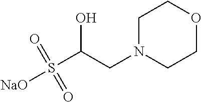

To a solution of aqueous HCl (12N, 1.90 mL, 2 eq) and H₂O (1 mL) at 0° C. was added 4-(2,2-dimethoxyethyl)morpholine (2 g, 11.41 mmol, 1 eq) dropwise, and the reaction mixture was stirred at 40° C. for 3 hours. The reaction was cooled to 0° C. and a suspension of Na₂SO₃ (1.94 g, 15.41 mmol, 1.35 eq) in H₂O (5 mL) was added maintaining the temperature below 20° C. After stirring for 16 hours at 20° C., the mixture was diluted with EtOH (10 mL), cooled to 0° C., and then stirred for 1 hour. The mixture was filtered and dried in vacuo to give (1-hydroxy-2-morpholino-ethyl)sulfonyloxysodium (1.36 g, 5.83 mmol, 51% yield) as a white solid. ¹H-NMR (400 MHz, D₂O) δ 4.91 (dd, J=10.4, 2.8 Hz, 1H), 3.98 (s, 4H), 3.62 (dd, J=13.6, 2.8 Hz, 1H), 3.58-3.28 (m, 5H).

### Step 3: Preparation of tert-butyl (E)-4-morpholinobut-2-enoate

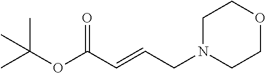 [SMILES: CC(C)(C)OC(=O)/C=C/CN1CCOCC1]

To a solution of (1-hydroxy-2-morpholino-ethyl)sulfonyloxysodium (1.33 g, 5.71 mmol, 1.2 eq) and tert-butyl 2-diethoxyphosphorylacetate (1.2 g, 4.76 mmol, 1 eq) in H₂O (3.6 mL) 0° C. was added aqueous NaOH (2.5 M, 7.14 mL, 3.75 eq) dropwise, and the reaction The mixture was stirred at 0° C. for 6 hours. The mixture was extracted with MTBE (3×60 mL), and the combined extract was washed with brine (30 mL), dried over anhydrous sodium sulfate, filtered, and concentrated. The resulting residue was purified by flash chromatography on SiO₂ (gradient: 0-15% EtOAc in petroleum ether) to afford tert-butyl (E)-4-morpholinobut-2-enoate (585 mg, 2.57 mmol, 54% yield) as a yellow oil. ¹H-NMR (400 MHz, CDCl₃) δ 6.90-6.77 (m, 1H), 5.91 (d, J=15.6 Hz, 1H), 3.79-3.65 (m, 4H), 3.10 (dd, J=6.4, 1.2 Hz, 2H), 2.50-2.40 (m, 4H), 1.49 (s, 9H).

### Step 4: Preparation of (E)-4-morpholinobut-2-enoic Acid

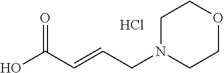 [SMILES: O=C(O)/C=C/CN1CCOCC1 ; Cl]

To a solution of tert-butyl (E)-4-morpholinobut-2-enoate (535 mg, 2.35 mmol, 1 eq) in EtOAc (5 mL) was added HCl (4N in dioxane, 17.65 mL, 30 eq), and the reaction mixture was stirred at 25° C. for 6 hours to give a white suspension. The mixture was concentrated, then diluted with i-Pr₂O (40 mL) and stirred for 2 minutes. The mixture was decanted, and the solid was dried in vacuo to give (E)-4-morpholinobut-2-enoic acid (435 mg, 1.68 mmol, 71% yield, HCl salt) as a white solid. ¹H-NMR (400 MHz, DMSO-d₆) δ 11.87 (br s, 1H), 6.93-6.80 (m, 1H), 6.17 (d, J=15.6 Hz, 1H), 4.00-3.75 (m, 6H), 3.35-3.25 (m, 3H), 3.12-2.94 (m, 2H).

### Step 4: Preparation of (2S,4R)-1-[(2S)-2-[[2-[[1-[2-[6-chloro-4-[(3S)-3-(cyanomethyl)-4-[(E)-4-morpholinobut-2-enoyl]piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-[(E)-4-morpholinobut-2-enoyl]piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-[(E)-4-(dimethylamino)but-2-enoyl]piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide using (E)-4-morpholinobut-2-enoic acid.

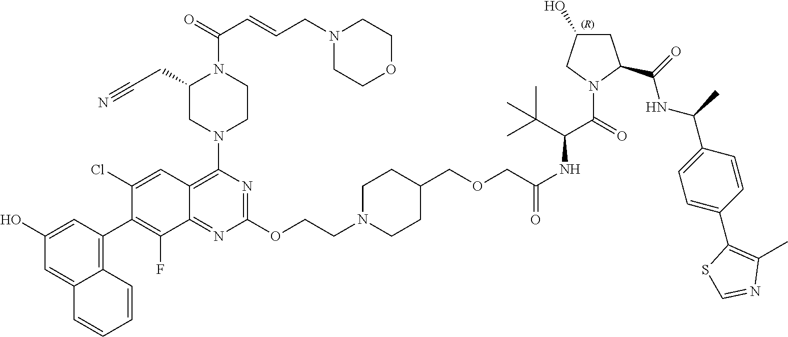

(formic acid salt, white solid). LC/MS (ESI) m/z: 1242.8 [M+H]⁺. ¹H-NMR (400 MHz, CDCl₃) δ 8.66 (s, 1H), 8.42 (s, 1H), 7.94-7.85 (m, 1H), 7.85-7.72 (m, 2H), 7.60-7.49 (m, 2H), 7.43-7.31 (m, 6H), 7.27-7.26 (m, 1H), 7.25-7.15 (m, 2H), 6.32-6.23 (m, 1H), 5.12-5.03 (m, 1H), 4.99-4.82 (m, 1H), 4.77-4.65 (m, 3H), 4.60-4.55 (m, 1H), 4.53-4.47 (m, 1H), 4.42-4.30 (m, 1H), 4.29-4.20 (m, 1H), 4.09-3.96 (m, 2H), 3.92-3.84 (m, 1H), 3.79-3.70 (m, 3H), 3.65-3.58 (m, 1H), 3.52-3.04 (m, 13H), 2.65-2.59 (m, 1H), 2.56-2.47 (m, 6H), 2.45-2.36 (m, 1H), 2.14-2.04 (m, 1H), 1.89-1.67 (m, 3H), 1.64-1.50 (m, 2H), 1.46 (t, J=6.4 Hz, 3H), 1.10-0.94 (m, 9H).

### Exemplary Synthesis of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

### Step 1: Preparation of (S)-tert-butyl (1-(4-bromophenyl)ethyl)carbamate

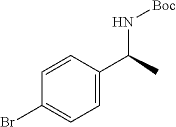 [SMILES: C[C@H](NC(=O)OC(C)(C)C)c1ccc(Br)cc1]

To a mixture of (1S)-1-(4-bromophenyl)ethanamine (5.0 g, 24.99 mmol, 1.0 eq) and NaHCO₃ (3.15 g, 37.49 mmol, 1.5 eq) in THF (50 mL) and water (15 mL) was added (Boc)₂₀ (6.54 g, 29.99 mmol, 1.2 eq), and the reaction mixture was stirred at 15° C. for 12 hours. Water (50 mL) was then added, and the resulting suspension was extracted with EtOAc (2×100 mL). The combined organic extracts were washed with brine (50 mL), dried over anhydrous Na₂SO₄, filtered, and concentrated. The resulting crude product was triturated with EtOAc/petroleum ether (40 mL, v/v=1:1) at 20° C. for 30 minutes. The suspension was filtered, and the filter cake was washed with petroleum ether (5 mL*2) and dried over vacuum to give tert-butyl N-[(1S)-1-(4-bromophenyl)ethyl]carbamate (5.1 g, 17 mmol, 68% yield) as a white solid. ¹H-NMR (400 MHz, DMSO-d₆) δ 7.49 (d, J=8.4 Hz, 2H), 7.43 (d, J=8.0 Hz, 1H), 7.24 (d, J=8.4 Hz, 2H), 4.57 (t, J=7.2 Hz, 1H), 1.35 (s, 9H), 1.27 (d, J=7.2 Hz, 3H).

### Step 2: Preparation of tert-butyl N-[(1S)-1-[4-(2-methylpyrazol-3-yl)phenyl]ethyl]carbamate

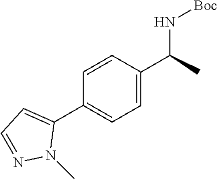 [SMILES: C[C@H](NC(=O)OC(C)(C)C)c1ccc(-c2ccnn2C)cc1]

To a solution of tert-butyl N-[(1S)-1-(4-bromophenyl)ethyl]carbamate (1.0 g, 3.33 mmol, 1.0 eq), (2-methylpyrazol-3-yl)boronic acid (503 mg, 4.00 mmol, 1.2 eq), and Na₂CO₃ (530 mg, 5.00 mmol, 1.5 eq) in dioxane (12 mL) and water (1 mL) was added Pd(dppf)C12 (244 mg, 333.12 umol, 0.1 eq), and the reaction mixture was stirred at 100° C. for 12 hours under N₂. The mixture was diluted with water (25 mL) and extracted with CH₂Cl₂ (3×45 mL). The combined organic extracts were washed with brine (50 mL), dried over Na₂SO₄, filtered, and concentrated. The resulting crude product was purified by flash silica gel chromatography (gradient: 0-40% EtOAc in petroleum ether) to afford tert-butyl N-[(1S)-1-[4-(2-methylpyrazol-3-yl)phenyl]ethyl]carbamate (840 mg, 2.68 mmol, 80% yield) as a yellow solid. LC/MS (ESI) m/z: 302.0 [M+H]⁺.

### Step 3: Preparation of (1S)-1-[4-(2-methylpyrazol-3-yl)phenyl]ethanamine

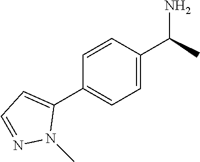

To tert-butyl N-[(1S)-1-[4-(2-methylpyrazol-3-yl)phenyl]ethyl]carbamate (840 mg, 2.79 mmol) in CH₂Cl₂ (5.0 mL) was added HCl (4N in dioxane solution, 3.0 mL), and the reaction mixture was stirred at 20° C. for 2 hours. The mixture was concentrated to give crude (1S)-1-[4-(2-methylpyrazol-3-yl)phenyl]ethanamine (651 mg HCl salt) as yellow foam, which would be directly used in the next step without further purification.

### Step 4: Preparation of tert-butyl(4R)-4-hydroxy-2-[[(1S)-1-[4-(2-methylpyrazol-3-yl)phenyl]ethyl]carbamoyl]pyrrolidine-1-carboxylate

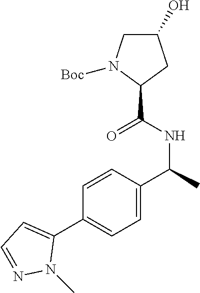 [SMILES: C[C@H](NC(=O)[C@@H]1C[C@@H](O)CN1C(=O)OC(C)(C)C)c1ccc(-c2ccnn2C)cc1]

To a solution of (2S,4R)-1-tert-butoxycarbonyl-4-hydroxy-pyrrolidine-2-carboxylic acid (696 mg, 3.01 mmol, 1.1 eq) in DMF (8.0 mL) was added diisopropylethylamine (1.41 g, 10.94 mmol, 4.0 eq) and HATU (1.25 g, 3.28 mmol, 1.2 eq), and the reaction mixture was stirred at 20° C. for 0.5 hour. (1S)-1-[4-(2-Methylpyrazol-3-yl)phenyl]ethanamine hydrochloride (650 mg, 2.73 mmol, 1.0 eq) was then added, and the reaction mixture was stirred at 20° C. for 12 hours. The mixture was diluted with water (50 mL) and extracted with EtOAc (4×80 mL). The combined extracts were washed with brine (100 mL), dried over anhydrous Na₂SO₄, filtered, and concentrated. The resulting crude product was purified by flash silica gel chromatography (gradient: of 0-8% CH₃OH in EtOAc) to afford tert-butyl(4R)-4-hydroxy-2-[[(1S)-1-[4-(2-methylpyrazol-3-yl)phenyl]ethyl]carbamoyl]pyrrolidine-1-carboxylate (684 mg, 1.64 mmol, 60% yield) as a yellow foam. LC/MS (ESI) m/z: 415.2 [M+H]⁺.

### Step 5: Preparation of (4R)-4-hydroxy-N-[(1S)-1-[4-(2-methylpyrazol-3-yl)phenyl]ethyl]pyrrolidine-2-carboxamide

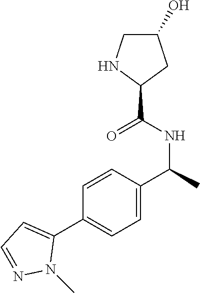 [SMILES: C[C@H](NC(=O)[C@@H]1C[C@@H](O)CN1)c1ccc(-c2ccnn2C)cc1]

To a solution of tert-butyl(4R)-4-hydroxy-2-[[(1S)-1-[4-(2-methylpyrazol-3-yl)phenyl]ethyl]carbamoyl]pyrrolidine-1-carboxylate (680 mg, 1.64 mmol) in CH₂Cl₂ (4.0 mL) was added HCl (4N in dioxane, 2.0 mL), and the reaction mixture was stirred at 20° C. for 1.5 hours. The mixture was concentrated over vacuum to give (4R)-4-hydroxy-N-[(1S)-1-[4-(2-methylpyrazol-3-yl)phenyl]ethyl]pyrrolidine-2-carboxamide (580 mg HCl salt, crude) as a yellow oil, which would be directly used in the next step without further purification.

### Step 6: Preparation of tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate

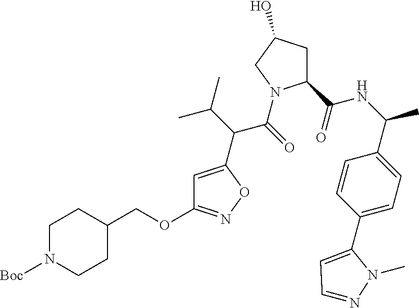 [SMILES: CC(C)C(C(=O)N1C[C@H](O)C[C@H]1C(=O)N[C@@H](C)c1ccc(-c2ccnn2C)cc1)c1cc(OCC2CCN(C(=O)OC(C)(C)C)CC2)no1]

To a solution of 2-[3-[(1-tert-butoxycarbonyl-4-piperidyl)methoxy]isoxazol-5-yl]-3-methyl-butanoic acid (3.00 g, 7.84 mmol, 1 eq) in CH₂Cl₂ (50 mL) were added diisopropylethylamine (5.07 g, 39.2 mmol, 6.83 mL, 5 eq) and HATU (3.88 g, 10.2 mmol, 1.3 eq), and the reaction mixture was stirred at 25° C. under N₂ for 10 minutes. (2S,4R)-4-Hydroxy-N-[(1S)-1-[4-(2-methylpyrazol-3-yl)phenyl]ethyl]pyrrolidine-2-carboxamide (2.75 g, 7.84 mmol, 1 eq, HCl) was then added, and the reaction mixture was stirred at 25° C. under N₂ for 3 hours. The reaction mixture was poured onto water (20 mL) and the layers were separated. The aqueous layer was further extracted with CH₂Cl₂ (20 mL). The combined organic extract was washed with brine (30 mL), dried over anhydrous Na₂SO₄, filtered, and concentrated. The resulting crude product was purified by flash silica gel chromatography (gradient: 0-90% EtOAc/petroleum ether) twice followed by prep-HPLC (45-62% CH₃CN in water (0.05% NH₄OH+10 mM NH₄HCO₃)) to give tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (1.41 g, 2.08 mmol, 27% yield) as a white solid. LC/MS (ESI) m/z: 679.5 [M+H]⁺.

### Step 7: Chiral Separation of tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate

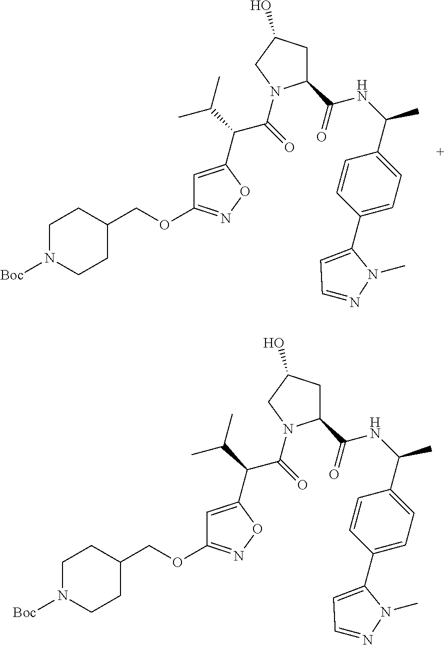 [SMILES: CC(C)[C@H](C(=O)N1C[C@H](O)C[C@H]1C(=O)N[C@@H](C)c1ccc(-c2ccnn2C)cc1)c1cc(OCC2CCN(C(=O)OC(C)(C)C)CC2)no1 ; CC(C)[C@@H](C(=O)N1C[C@H](O)C[C@H]1C(=O)N[C@@H](C)c1ccc(-c2ccnn2C)cc1)c1cc(OCC2CCN(C(=O)OC(C)(C)C)CC2)no1]

tert-Butyl4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (2.10 g, 3.09) was separated by SFC (35% isopropanol in water (0.1% NH₄OH) to give tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (522 mg, 0.769 mmol, 25% yield) as a white solid (LC/MS (ESI) m/z: 679.5 [M+H]⁺) and tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (1.52 g, 2.24 mmol, 72% yield) as a white solid (LC/MS (ESI) m/z: 679.5 [M+H]⁺).

### Step 8: Preparation of (2S,4R)-4-hydroxy-1-[(2R)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide

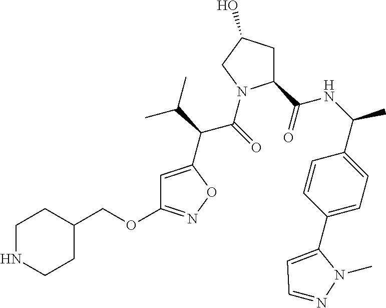 [SMILES: CC(C)[C@@H](C(=O)N1C[C@H](O)C[C@H]1C(=O)N[C@@H](C)c1ccc(-c2ccnn2C)cc1)c1cc(OCC2CCNCC2)no1]

To a solution of tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (1.00 g, 1.47 mmol, 1 eq) in CH₃OH (10 mL) was added HCl (4N in dioxane, 10 mL), and the reaction mixture was stirred at 25° C. for 1 hour. The reaction was concentrated, and the resulting residue was taken up in water. The pH of the resulting aqueous mixture was adjusted to ˜8 by addition of NaHCO₃. The aqueous mixture was extracted with 10:1 CH₂Cl₂/MeOH (3×20 mL), and the combined organic extracts were washed with brine (2×15 mL), dried over anhydrous Na₂SO₄, filtered, and concentrated to give (2S,4R)-4-hydroxy-1-[(2R)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide (836 mg, 1.44 mmol, 98% yield) as a yellow solid. LC/MS (ESI) m/z: 579.5 [M+H]⁺.

### Step 9: Preparation of (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

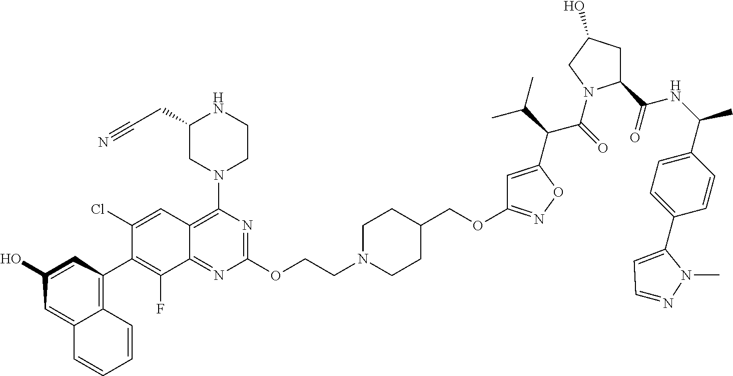

To a solution of (2S,4R)-4-hydroxy-1-[(2R)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide (1.04 g, 1.80 mmol, 1 eq) and 2-[(2S)-4-[6-chloro-8-fluoro-7-(3-hydroxy-1-naphthyl)-2-(2-oxoethoxy)quinazolin-4-yl]piperazin-2-yl]acetonitrile (956 mg, 1.89 mmol, 1.05 eq) in CH₂Cl₂ (25 mL) and isopropanol (25 mL) were added AcOH (432 mg, 7.20 mmol, 0.412 mL, 4 eq) and 2-picolineborane complex (963 mg, 9.00 mmol, 5 eq), and the reaction mixture was stirred at 25° C. under N₂ for 45 minutes. The mixture was concentrated, and the resulting residue was purified by prep-HPLC (25-70% CH₃CN in water (10 mM NH₄HCO₃)) to give (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide (4.04 g, 3.78 mmol, 53% yield) as a yellow solid. LC/MS (ESI) m/z: 1068.4 [M+H]⁺.

### Step 10: Preparation of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

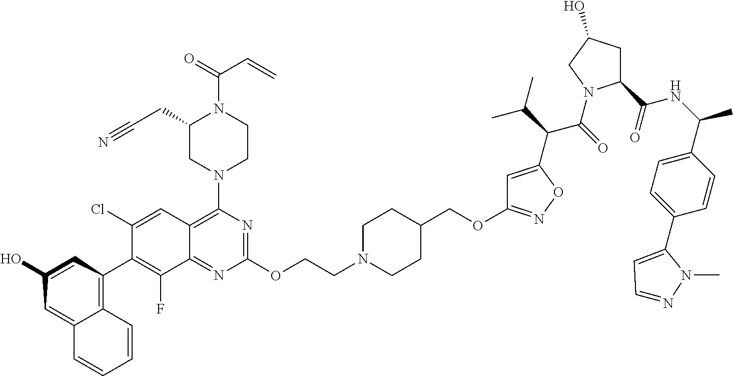

To a solution of (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide (2.02 g, 1.74 mmol, 92% purity, 1 eq) and 2,6-dimethylpyridine (559 mg, 5.22 mmol, 0.608 mL, 3 eq) in CH₂Cl₂ (20 mL) at −78° C. was added prop-2-enoyl chloride (173 mg, 1.91 mmol, 0.156 mL, 1.1 eq), and the reaction mixture was stirred at −78° C. under N₂ for 0.5 hour. The reaction mixture was concentrated, and the resulting crude product was purified by prep-HPLC (10-45% CH₃CN in water (0.225% formic acid)) to give (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide (966.8 mg, 0.790 mmol, 45% yield, formic acid salt) as a white solid. LC/MS (ESI) m/z: 1122.6 [M+H]⁺. ¹H-NMR (400 MHz, CD₃OD) δ 8.47 (s, 1H), 8.10 (s, 1H), 7.75 (d, J=8.4 Hz, 1H), 7.52-7.35 (m, 6H), 7.31-7.13 (m, 3H), 7.03 (d, J=2.4 Hz, 1H), 6.98-6.74 (m, 1H), 6.38-6.26 (m, 2H), 6.00-5.92 (m, 1H), 5.85 (d, J=10.0 Hz, 1H), 5.16-4.94 (m, 2H), 4.81-4.75 (m, 2H), 4.56-4.37 (m, 4H), 4.26-3.99 (m, 3H), 3.90-3.72 (m, 6H), 3.67 (d, J=9.6 Hz, 1H), 3.65-3.43 (m, 4H), 3.39-3.32 (m, 2H), 3.12-2.93 (m, 2H), 2.90-2.67 (m, 2H), 2.43-2.31 (m, 1H), 2.23-2.14 (m, 1H), 2.11-1.87 (m, 4H), 1.67-1.45 (m, 5H), 1.05 (d, J=6.4 Hz, 3H), 0.94-0.81 (m, 3H).

### Exemplary Synthesis of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

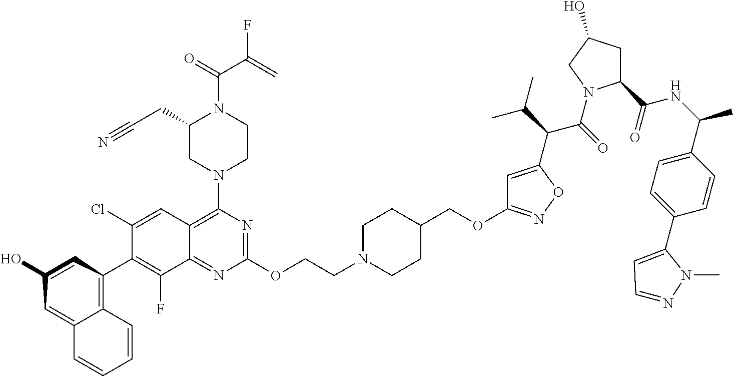

To (2S,4R)-1-[(2R)-2-[3-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide (2.00 g, 1.87 mmol, 1.0 eq) in DMF (20 mL) were added diisopropylethylamine (978 uL, 5.61 mmol, 3.0 eq) and HATU (2.13 g, 5.61 mmol, 3.0 eq) followed by slow addition of 2-fluoroprop-2-enoic acid (202.25 mg, 2.25 mmol, 1.2 eq), and the reaction mixture was stirred at 25° C. for 2 hours. Saturated aqueous K₂CO₃ aqueous (30 mL) was then added, and the resulting mixture was stirred for 30 minutes. The aqueous mixture was extracted with EtOAc (3×30 mL), and the combined organic layers were dried over Na₂SO₄, filtered, and concentrated. The resulting residue was purified by prep-HPLC (CH₃CN in water (0.225% formic acid)) to afford (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide (1.06 g, 0.85 mmol, 46% yield, formic acid salt) as a white solid. LC/MS (ESI) m/z: 1140.4 [M+H]⁺. ¹H-NMR (400 MHz, DMSO-d₆) δ 8.43 (d, J=7.6 Hz, 1H), 8.11 (s, 1H), 7.81 (d, J=8.4 Hz, 1H), 7.50-7.43 (m, 4H), 7.40-7.36 (m, 2H), 7.29 (d, J=2.4 Hz, 1H), 7.22 (d, J=4.0 Hz, 2H), 7.07 (d, J=2.0 Hz, 1H), 6.37 (d, J=2.0 Hz, 1H), 6.06 (s, 1H), 5.46-5.23 (m, 2H), 5.11 (s, 1H), 4.97-4.86 (m, 2H), 4.49 (s, 2H), 4.40-4.33 (m, 2H), 4.31-4.26 (m, 2H), 3.98 (d, J=6.0 Hz, 2H), 3.84 (s, 3H), 3.74-3.59 (m, 7H), 3.11-2.96 (m, 3H), 2.78 (s, 2H), 2.29-1.95 (m, 5H), 1.81-1.66 (m, 4H), 1.48-1.20 (m, 6H), 0.98-0.93 (m, 3H), 0.82-0.75 (m, 3H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide

### Step 1: Preparation of tert-butyl N-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]carbamate

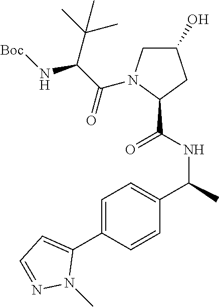 [SMILES: C[C@H](NC(=O)[C@@H]1C[C@@H](O)CN1C(=O)[C@@H](NC(=O)OC(C)(C)C)C(C)(C)C)c1ccc(-c2ccnn2C)cc1]

To a mixture of (2S,4R)-4-hydroxy-N-[(1S)-1-[4-(2-methylpyrazol-3-yl)phenyl]ethyl]pyrrolidine-2-carboxamide (9.30 g, 26.5 mmol, 1 eq, HCl salt), diisopropylethylamine (17.07 g, 132.05 mmol, 23 mL, 4.98 eq), and (2S)-2-(tert-butoxycarbonylamino)-3,3-dimethylbutanoic acid (7.36 g, 31.81 mmol, 1.2 eq) in DMF (200 mL) was added HATU (15.1 g, 39.8 mmol, 1.5 eq) in one portion at 0° C., and the reaction mixture was stirred at 20° C. for 12 hours. The mixture was poured onto water (300 mL), and the resulting mixture was extracted with 10:1 CH₂Cl₂/CH₃OH (3×300 mL). The combined organic extract was washed with brine (2×300 mL), dried over anhydrous Na₂SO₄, filtered, and concentrated. The resulting residue was purified by flash chromatography on SiO₂ (gradient: 0-70% THF in petroleum ether) to give tert-butyl N-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]carbamate (12.5 g, 23.69 mmol, 89% yield) as a white solid. LC/MS (ESI) m/z: 528.5 [M+H]⁺.

### Step 2: Preparation of (2S,4R)-1-[(2S)-2-amino-3,3-dimethyl-butanoyl]-4-hydroxy-N-[(1S)-1-[4-(2-methylpyrazol-3-yl)phenyl]ethyl]pyrrolidine-2-carboxamide

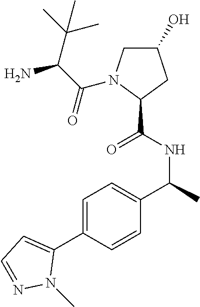 [SMILES: C[C@H](NC(=O)[C@@H]1C[C@@H](O)CN1C(=O)[C@@H](N)C(C)(C)C)c1ccc(-c2ccnn2C)cc1]

To a mixture of tert-butyl N-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]carbamate (12.5 g, 23.7 mmol, 1 eq) in CH₃OH (30 mL) was added HCl (4N in CH₃OH, 30 mL) in one portion at 20° C., and the reaction mixture was stirred at 20° C. for 30 minutes. The mixture was concentrated under reduced pressure to give (2S,4R)-1-[(2S)-2-amino-3,3-dimethyl-butanoyl]-4-hydroxy-N-[(1S)-1-[4-(2-methylpyrazol-3-yl)phenyl]ethyl]pyrrolidine-2-carboxamide (10.9 g, 23.5 mmol, 99% yield, HCl salt) as a white solid. LC/MS (ESI) m/z: 428.2 [M+H]⁺.

### Step 3: Preparation of tert-butyl 4-[[2-[[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]amino]-2-oxoethoxy]methyl]piperidine-1-carboxylate

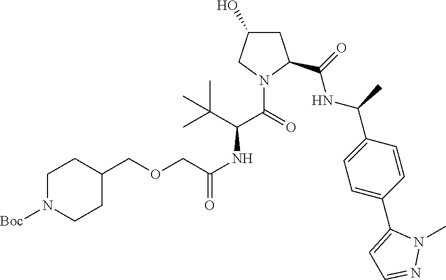 [SMILES: C[C@H](NC(=O)[C@@H]1C[C@@H](O)CN1C(=O)[C@@H](NC(=O)COCC1CCN(C(=O)OC(C)(C)C)CC1)C(C)(C)C)c1ccc(-c2ccnn2C)cc1]

To a solution of 2-[(1-tert-butoxycarbonyl-4-piperidyl)methoxy]acetic acid (6.00 g, 22.0 mmol, 1.1 eq) in CH₂Cl₂ (120 mL) were added HATU (15.2 g, 39.9 mmol, 2 eq) and diisopropylethylamine (12.9 g, 99.8 mmol, 17.4 mL, 5 eq), and the resulting mixture was stirred at 25° C. for 0.5 hour. (2S,4R)-1-[(2S)-2-Amino-3,3-dimethyl-butanoyl]-4-hydroxy-N-[(1S)-1-[4-(2-methylpyrazol-3-yl)phenyl]ethyl]pyrrolidine-2-carboxamide (9.26 g, 20.0 mmol, 1 eq, HCl salt) was then added, and the reaction mixture was stirred at 25° C. for 1 hour. The mixture was concentrated, and the resulting residue was purified by flash chromatography on SiO₂ (gradient: 0-13% CH₃OH in EtOAc) to give tert-butyl 4-[[2-[[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]amino]-2-oxoethoxy]methyl]piperidine-1-carboxylate (5.10 g, 6.72 mmol, 34% yield) as a yellow oil. LC/MS (ESI) m/z: 683.6 [M+H]⁺.

### Step 4: Preparation of (2S,4R)-1-[(2S)-3,3-dimethyl-2-[[2-(4-piperidylmethoxy)acetyl]amino]butanoyl]-4-hydroxy-N-[(1S)-1-[4-(2-methylpyrazol-3-yl)phenyl]ethyl]pyrrolidine-2-carboxamide

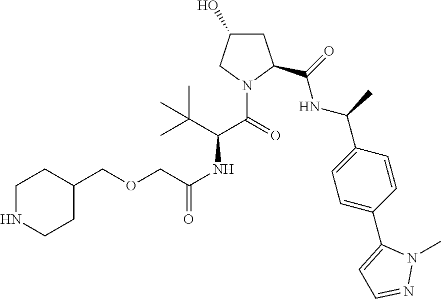 [SMILES: C[C@H](NC(=O)[C@@H]1C[C@@H](O)CN1C(=O)[C@@H](NC(=O)COCC1CCNCC1)C(C)(C)C)c1ccc(-c2ccnn2C)cc1]

To a solution of tert-butyl 4-[[2-[[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]amino]-2-oxo-ethoxy]methyl]piperidine-1-carboxylate (5.10 g, 7.47 mmol, 1 eq) in CH₂Cl₂ (40 mL) was added HCl (4N in dioxane, 15 mL), and the reaction mixture was stirred at 25° C. for 2 hours. The mixture was adjusted to pH-8 by addition of saturated aqueous NaHCO₃ and then extracted with CH₂Cl₂ (150 mL). The organic extract was dried over Na₂SO₄, filtrated, and concentrated to give (2S,4R)-1-[(2S)-3,3-dimethyl-2-[[2-(4-piperidylmethoxy)acetyl]amino]butanoyl]-4-hydroxy-N-[(1S)-1-[4-(2-methylpyrazol-3-yl)phenyl]ethyl]pyrrolidine-2-carboxamide (2.7 g, 4.63 mmol, 62% yield) as a yellow solid. LC/MS (ESI) m/z: 583.4 [M+H]⁺.

### Step 5: Preparation of (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide

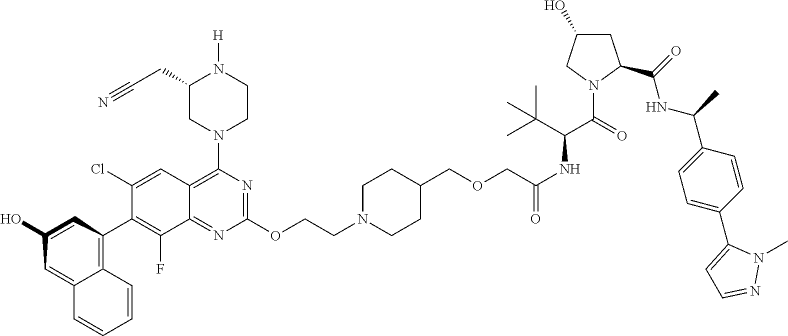

To a mixture of 2-[(2S)-4-[6-chloro-8-fluoro-7-(3-hydroxy-1-naphthyl)-2-(2-oxoethoxy)quinazolin-4-yl]piperazin-2-yl]acetonitrile (1.10 g, 2.17 mmol, 1.1 eq) and (2S,4R)-1-[(2S)-3,3-dimethyl-2-[[2-(4-piperidylmethoxy)acetyl]amino]butanoyl]-4-hydroxy-N-[(1S)-1-[4-(2-methylpyrazol-3-yl)phenyl]ethyl]pyrrolidine-2-carboxamide (1.15 g, 1.97 mmol, 1 eq) in CH₂Cl₂ (25 mL) and isopropanol (25 mL) were added AcOH (0.452 mL, 7.90 mmol, 4 eq) and 2-picoline borane complex (1.06 g, 9.86 mmol, 5 eq) in one portion at 20° C., and the reaction mixture was stirred at 20° C. for 1 hour. The mixture was concentrated, and the resulting residue was purified by prep-HPLC (CH₃CN in water (0.05% NH₄OH+10 mM NH₄HCO₃)) to give (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide (1.13 g, 0.695 mmol, 66% pure) as a white solid. LC/MS (ESI) m/z: 536.8[M/2+H]⁺.

### Step 6: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide

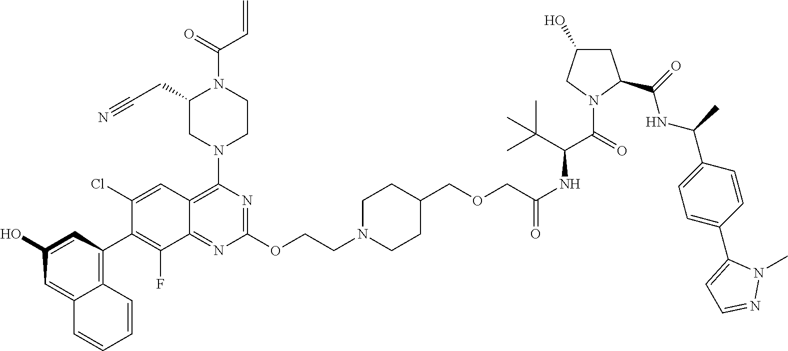

To a mixture of (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide (1.13 g, 0.695 mmol, 66% purity, 1 eq) and 2,6-dimethylpyridine (372 mg, 3.47 mmol, 0.404 mL, 4.99 eq) in CH₂Cl₂ (50 mL) −78° C. was added prop-2-enoyl chloride (0.062 mL, 0.76 mmol, 1.09 eq) dropwise under N₂, and the reaction mixture was stirred at −78° C. for 30 minutes. Water (0.5 mL) was then added, and the resulting mixture was warmed to 20° C. The mixture was concentrated, and the resulting residue was purified by prep-HPLC (CH₃CN in water (0.225% formic acid)) to give (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide (350 mg, 0.298 mmol, formic acid salt) as a white solid. LC/MS (ESI) m/z: 1126.8 [M+H]⁺. ¹H-NMR (400 MHz, DMSO-d₆) δ 8.46 (d, J=7.6 Hz, 1H), 8.24 (s, 2H), 8.11 (s, 1H), 7.81 (d, J=8.4 Hz, 1H), 7.50-7.42 (m, 5H), 7.40-7.35 (m, 2H), 7.31-7.26 (m, 2H), 7.23 (d, J=4.0 Hz, 2H), 7.08 (d, J=2.4 Hz, 1H), 6.98-6.78 (m, 1H), 6.37 (d, J=2.0 Hz, 1H), 6.22 (d, J=16.4 Hz, 1H), 5.80 (d, J=9.2 Hz, 1H), 4.99 (d, J=2.4 Hz, 1H), 4.95-4.85 (m, 2H), 4.71-4.63 (m, 1H), 4.54 (d, J=9.6 Hz, 1H), 4.50-4.42 (m, 2H), 4.41-4.31 (m, 2H), 4.25 (d, J=15.6 Hz, 3H), 4.16-4.03 (m, 2H), 3.91 (s, 2H), 3.84 (s, 5H), 3.12-3.03 (m, 3H), 3.01-2.92 (m, 3H), 2.71 (t, J=5.6 Hz, 3H), 2.12-1.98 (m, 2H), 1.76-1.53 (m, 3H), 1.36 (d, J=7.2 Hz, 3H), 1.27-1.14 (m, 2H), 0.93 (s, 9H).

### Exemplary Synthesis of (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide

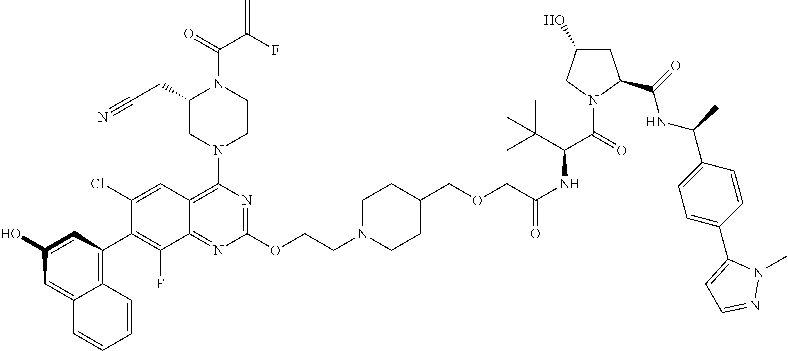

To a mixture of (2S,4R)-1-[(2S)-2-[[2-[[1-[2-[6-chloro-4-[(3S)-3-(cyanomethyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide (2.33 g, 2.17 mmol, 1 eq), diisopropylethylamine (842 mg, 6.52 mmol, 3 eq), and 2-fluoroprop-2-enoic acid (293 mg, 3.25 mmol, 1.5 eq) in DMF (30 mL) was added HATU (2.48 g, 6.52 mmol, 3 eq) in one portion at 0° C., and the reaction mixture was stirred at 20° C. for 1.5 hours. Saturated aqueous K₂CO₃ (30 mL) was then added, and the resulting mixture was stirred at 20° C. for 30 minutes. The mixture was poured onto water (100 mL) and extracted with 1:1 EtOAc/THF (3×100 mL). The combined organic extract was washed with brine (2×100 mL), dried over anhydrous Na₂SO₄, filtered, and concentrated. The resulting residue was purified by prep-HPLC (CH₃CN in water (0.225% formic acid)) to afford I (2S,4R)-1-[(2S)-2-[[2-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(2-methylpyrazol-3-yl)phenyl]ethyl]pyrrolidine-2-carboxamide (973.5 mg, 0.794 mmol, 37% yield, formic acid salt) as a white solid. LC/MS (ESI) m/z: 1144.8 [M+H]⁺. ¹H-NMR (400 MHz, DMSO-d₆) δ 10.04 (s, 1H), 8.47 (d, J=7.6 Hz, 1H), 8.14 (s, 1H), 8.12 (s, 1H), 7.82 (d, J=8.4 Hz, 1H), 7.50-7.42 (m, 5H), 7.37 (d, J=8.4 Hz, 2H), 7.32-7.27 (m, 3H), 7.22 (d, J=4.0 Hz, 2H), 7.07 (d, J=2.4 Hz, 1H), 6.37 (d, J=2.0 Hz, 1H), 5.48-5.36 (m, 2H), 5.32-5.23 (m, 1H), 5.14 (d, J=3.2 Hz, 1H), 4.96-4.86 (m, 2H), 4.57-4.24 (m, 10H), 3.92 (s, 3H), 3.84 (s, 4H), 3.62-3.53 (m, 2H), 3.11-2.97 (m, 3H), 2.84-2.72 (m, 3H), 2.20-1.99 (m, 2H), 1.84-1.56 (m, 4H), 1.36 (d, J=7.2 Hz, 3H), 1.29-1.14 (m, 2H), 0.93 (s, 9H).

### Exemplary Synthesis of (2S,4R)-1-[(2R)-2-[3-[7-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of methyl 3-methyl-2-[3-(1,1,2,2,3,3,4,4,4-nonafluorobutylsulfonyloxy)isoxazol-5-yl]butanoate

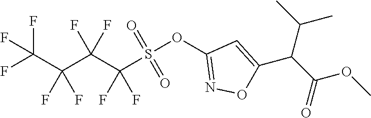 [SMILES: COC(=O)C(c1cc(OS(=O)(=O)C(F)(F)C(F)(F)C(F)(F)C(F)(F)F)no1)C(C)C]

To a mixture of methyl 2-(3-hydroxyisoxazol-5-yl)-3-methyl-butanoate (20.0 g, 100 mmol, 1 eq) and K₂CO₃ (6.94 g, 50.2 mmol, 0.5 eq) in CH₃CN (100 mL) at 0° C. was added 1,1,2,2,3,3,4,4,4-nonafluorobutane-1-sulfonyl fluoride (60.7 g, 201 mmol, 2 eq) dropwise under N₂, and the reaction mixture was stirred at 20° C. for 12 hours. The mixture was diluted with EtOAc (100 mL) and washed with saturated NH₄Cl solution (100 mL). The aqueous phase was extracted with EtOAc (3×120 mL), and the organic extract was washed with brine (100 mL), dried over anhydrous Na₂SO₄, and concentrated. The resulting residue was purified by flash chromatography on SiO₂ (gradient: 0-1% THF in petroleum ether) to afford methyl 3-methyl-2-[3-(1,1,2,2,3,3,4,4,4-nonafluorobutylsulfonyloxy)isoxazol-5-yl]butanoate (37.4 g, 71.5 mmol, 71% yield) as a colorless oil. LC/MS (ESI) m/z: 481.8 [M+H]⁺.

### Step 2: Preparation of tert-butyl 2-[5-(1-methoxycarbonyl-2-methyl-propyl) isoxazol-3-yl]-2, 7-diazaspiro[3.5]nonane-7-carboxylate

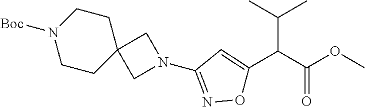 [SMILES: COC(=O)C(c1cc(N2CC3(CCN(C(=O)OC(C)(C)C)CC3)C2)no1)C(C)C]

To a solution of tert-butyl 2,7-diazaspiro[3.5]nonane-7-carboxylate (3.00 g, 13.3 mmol, 1 eq) in DMA (60 mL) were added diisopropylethylamine (5.14 g, 39.8 mmol, 3 eq) and methyl 3-methyl-2-[3-(1,1,2,2,3,3,4,4,4-nonafluorobutylsulfonyloxy)isoxazol-5-yl]butanoate (7.66 g, 15.9 mmol, 1.2 eq), and the reaction mixture was stirred at 140° C. for 12 hours under N₂. The resulting residue was purified by prep-HPLC (30-70% CH₃CN in water (0.05% HCl)) to afford tert-butyl 2-[5-(1-methoxycarbonyl-2-methyl-propyl)isoxazol-3-yl]-2,7-diazaspiro[3.5]nonane-7-carboxylate (1.13 g, 2.63 mmol, 20% yield) as a white solid. LC/MS (ESI) m/z: 408.0 [M+H]⁺.

### Step 3: Preparation of 2-[3-(7-tert-butoxycarbonyl-2, 7-diazaspiro[3.5]nonan-2-yl) isoxazol-5-yl]-3-methyl-butanoic Acid

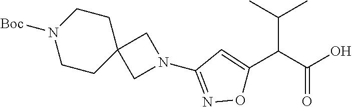 [SMILES: CC(C)C(C(=O)O)c1cc(N2CC3(CCN(C(=O)OC(C)(C)C)CC3)C2)no1]

To tert-butyl 2-[5-(1-methoxycarbonyl-2-methyl-propyl) isoxazol-3-yl]-2, 7-diazaspiro[3.5]nonane-7-carboxylate (1.13 g, 2.77 mmol, 1 eq) in THF (10 mL), CH₃OH (10 mL) and H₂O (10 mL) was added LiOH.H₂O (582 mg, 13.9 mmol, 5 eq), and the reaction mixture was stirred at 20° C. for 1 hour. The reaction mixture was adjusted to pH=3 with aq. HCl (2N), and then lyophilized to give 2-[3-(7-tert-butoxycarbonyl-2, 7-diazaspiro[3.5]nonan-2-yl) isoxazol-5-yl]-3-methyl-butanoic acid (1.09 g, crude) as a white solid. LC/MS (ESI) m/z: 394.0 [M+H]⁺.

### Step 4: Preparation of tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate

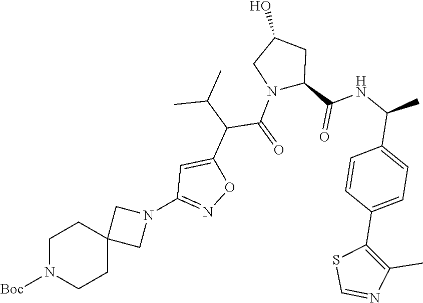 [SMILES: Cc1ncsc1-c1ccc([C@H](C)NC(=O)[C@@H]2C[C@@H](O)CN2C(=O)C(c2cc(N3CC4(CCN(C(=O)OC(C)(C)C)CC4)C3)no2)C(C)C)cc1]

To a mixture of 2-[3-(7-tert-butoxycarbonyl-2,7-diazaspiro[3.5]nonan-2-yl)isoxazol-5-yl]-3-methyl-butanoic acid (1.09 g, 2.77 mmol, 1 eq) and (2S,4R)-4-hydroxy-N-[(1S)-1-[4-(4-methylthiazol-5-yl)phenyl]ethyl]pyrrolidine-2-carboxamide (1.02 g, 2.77 mmol, 1 eq, HCl salt) in DMF (10 mL) were added diisopropylethylamine (1.79 g, 13.9 mmol, 5 eq) and HATU (1.05 g, 2.77 mmol, 1 eq), and the reaction mixture was stirred at 20° C. for 2 hours. Water (20 mL) was then added, and the resulting mixture was extracted with EtOAc (3×20 mL). The combined organic extract was washed with brine (3×20 mL), dried over Na₂SO₄, and concentrated. The resulting residue was purified by flash chromatography on SiO₂ (gradient: 0-50% THF in petroleum ether) to afford tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (1.96 g, 2.33 mmol, 84% purity) as a yellow solid. LC/MS (ESI) m/z: 707.5 [M+H]⁺.

### Step 5: Chiral separation of tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate

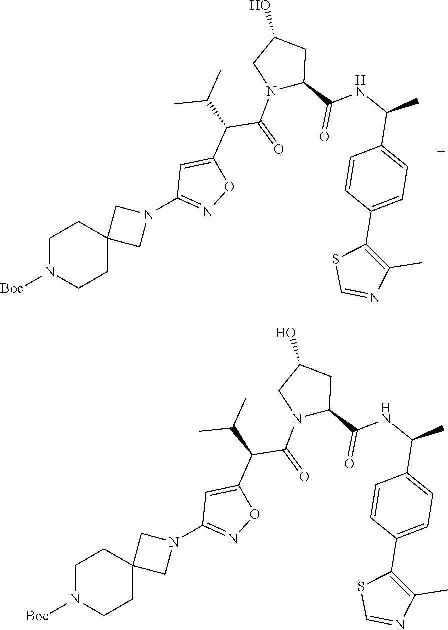 [SMILES: Cc1ncsc1-c1ccc([C@H](C)NC(=O)[C@@H]2C[C@@H](O)CN2C(=O)[C@H](c2cc(N3CC4(CCN(C(=O)OC(C)(C)C)CC4)C3)no2)C(C)C)cc1 ; Cc1ncsc1-c1ccc([C@H](C)NC(=O)[C@@H]2C[C@@H](O)CN2C(=O)[C@@H](c2cc(N3CC4(CCN(C(=O)OC(C)(C)C)CC4)C3)no2)C(C)C)cc1]

tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (1.70 g, 2.40 mmol, 1 eq) was separated by SFC (40% isopropanol in water (0.1% NH₄OH)) to afford tert-butyl 2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (800 mg, 0.985 mmol, 41% yield, 87% purity) as a white solid (LC/MS (ESI) m/z: 707.3 [M+H]⁺) and tert-butyl 2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (760 mg, 1.02 mmol, 42% yield) as a white solid (LC/MS (ESI) m/z: 707.4 [M+H]⁺).

### Step 6: Preparation of (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide

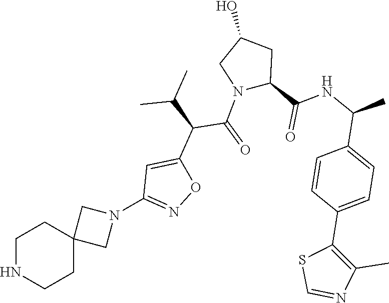 [SMILES: Cc1ncsc1-c1ccc([C@H](C)NC(=O)[C@@H]2C[C@@H](O)CN2C(=O)[C@@H](c2cc(N3CC4(CCNCC4)C3)no2)C(C)C)cc1]

To a solution of tert-butyl 2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (760 mg, 1.08 mmol, 1 eq) in CH₂Cl₂ (3 mL) was added TFA (1 mL), and the reaction mixture was stirred at 20° C. under N₂ for 2.5 hours. The reaction mixture was concentrated under reduced pressure, and the resulting residue was diluted with water (10 mL). The resulting aqueous mixture was adjusted to pH=9 with saturated aqueous NaHCO₃ and extracted with 20:1 CH₂Cl₂/CH₃OH (3×20 mL). The combined organic extract was dried over anhydrous Na₂SO₄, filtered, and concentrated to give (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (646 mg, 0.905 mmol, 85% purity) as a white solid. LC/MS (ESI) m/z: 607.4 [M+H]⁺.

### Step 7: Preparation of (2S,4R)-1-[(2R)-2-[3-[7-[2-[6-chloro-4-[(3S)-3-(cyanomethyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

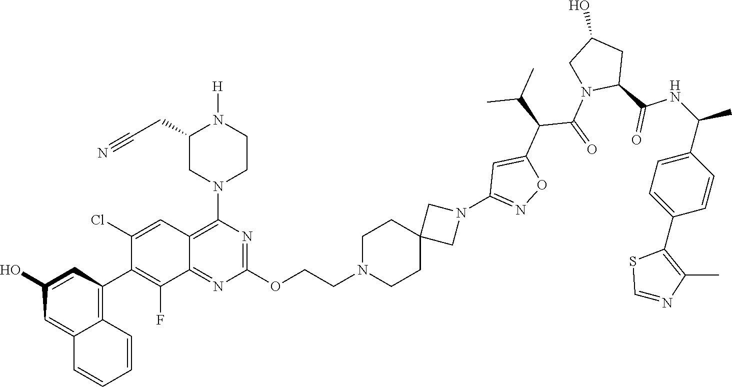

To a solution of 2-[(2S)-4-[6-chloro-8-fluoro-7-(3-hydroxy-1-naphthyl)-2-(2-oxoethoxy)quinazolin-4-yl]piperazin-2-yl]acetonitrile (549.40 mg, 1.09 mmol, 1.2 eq) and (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (646 mg, 0.905 mmol, 85% purity, 1 eq) in CH₂Cl₂ (20 mL) and isopropanol (20 mL) were added AcOH (217 mg, 3.62 mmol, 4 eq) and 2-picolineborane complex (484 mg, 4.52 mmol, 5 eq), and the reaction mixture was stirred at 20° C. for 1 hour. The mixture was concentrated, and the resulting crude product was purified by prep-HPLC (40-85% CH₃CN in water (10 mM NH₄HCO₃)) to give (2S,4R)-1-[(2R)-2-[3-[7-[2-[6-chloro-4-[(3S)-3-(cyanomethyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (366 mg, 0.334 mmol, 37% yield) as a white solid. LC/MS (ESI) m/z: 549.1 [M/2+H]⁺.

### Step 8: Preparation of (2S,4R)-1-[(2R)-2-[3-[7-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

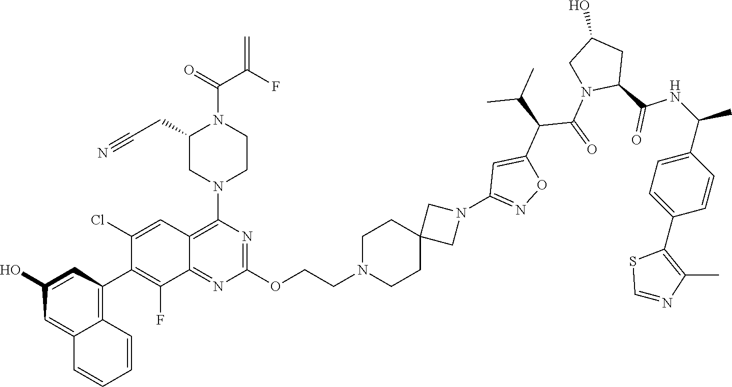

To a solution of (2S,4R)-1-[(2R)-2-[3-[7-[2-[6-chloro-4-[(3S)-3-(cyanomethyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (366 mg, 0.334 mmol, 1 eq), 2-fluoroprop-2-enoic acid (45 mg, 0.50 mmol, 1.5 eq), and diisopropylethylamine (129 mg, 1.00 mmol, 3 eq) in DMF (5 mL) was added HATU (381 mg, 1.00 mmol, 3 eq), and the reaction mixture was stirred at 25° C. for 2 hours. Saturated aqueous K₂CO₃ (5 mL) was then added, and the resulting mixture was stirred at 25° C. for 0.5 hour. The mixture was extracted with CH₂Cl₂ (3×5 mL), and the combined organic extracts was dried over anhydrous Na₂SO₄, filtered, and concentrated. The resulting crude product was purified by prep-HPLC (20-72% CH₃CN in water (0.225% formic acid)) to give the desired product (88 mg) as a white solid. Further purification of impure fractions by prep-HPLC (10-60% CH₃CN in water (0.225% formic acid)) gave additional desired product. Purified material was combined to give (2S,4R)-1-[(2R)-2-[3-[7-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (168.2 mg, 0.132 mmol, 40% yield, formic acid salt). LC/MS (ESI) m/z: 585.4 [M/2+H]⁺. ¹H-NMR (400 MHz, CD₃OD) δ 8.90-8.60 (m, 1H), 8.43 (s, 1H), 8.08 (s, 1H), 7.76 (d, J=8.4 Hz, 1H), 7.49-7.33 (m, 5H), 7.30-7.14 (m, 3H), 7.03 (d, J=2.4 Hz, 1H), 5.88-5.81 (m, 1H), 5.47-5.27 (m, 2H), 5.06-5.00 (m, 1H), 4.74 (t, J=4.8 Hz, 2H), 4.57-4.41 (m, 4H), 3.88-3.79 (m, 2H), 3.75-3.67 (m, 5H), 3.66-3.43 (m, 3H), 3.25-3.17 (m, 2H), 3.16-2.88 (m, 6H), 2.50-2.45 (m, 3H), 2.41-2.31 (m, 1H), 2.22-2.12 (m, 1H), 2.08-1.91 (m, 5H), 1.61-1.49 (m, 3H), 1.41-1.18 (m, 2H), 1.04 (d, J=6.4 Hz, 3H), 0.92-0.84 (m, 3H).

### Exemplary Synthesis of (2S,4R)-1-[(2R)-2-[3-[7-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

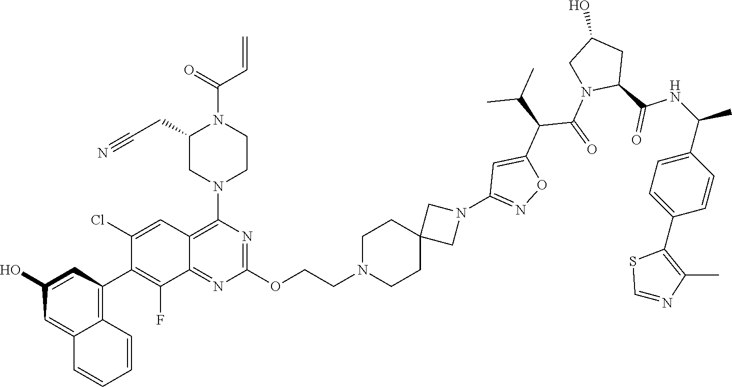

To a solution of (2S,4R)-1-[(2R)-2-[3-[7-[2-[6-chloro-4-[(3S)-3-(cyanomethyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (480 mg, 0.438 mmol, 1 eq) and 2,6-dimethylpyridine (141 mg, 1.31 mmol, 3 eq) in CH₂Cl₂ (30 mL) at −78° C. was added prop-2-enoyl chloride (44 mg, 0.48 mmol, 1.1 eq), and the reaction mixture was stirred at −78° C. under N₂ for 0.5 hours. The reaction mixture was quenched by addition of water (1 mL) and concentrated under reduced pressure to give crude product. Purification by prep-HPLC (30-60% CH₃CN in water (10 mM NH₄HCO₃)) afforded (2S,4R)-1-[(2R)-2-[3-[7-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (232 mg, 0.195 mmol, 45% yield) as a white solid. LC/MS (ESI) m/z: 1150.5 [M+H]⁺. ¹H-NMR (400 MHz, CD₃OD) δ 8.89-8.84 (m, 1H), 8.06 (s, 1H), 7.75 (d, J=8.0 Hz, 1H), 7.50-7.34 (m, 5H), 7.29-7.15 (m, 3H), 7.04 (d, J=2.4 Hz, 1H), 6.96-6.75 (m, 1H), 6.31 (d, J=17.6 Hz, 1H), 5.89-5.78 (m, 2H), 5.05-4.99 (m, 1H), 4.64 (t, J=5.2 Hz, 2H), 4.49-4.37 (m, 3H), 4.28-4.06 (m, 1H), 3.90-3.79 (m, 2H), 3.76-3.43 (m, 9H), 3.08-2.97 (m, 2H), 2.91-2.81 (m, 2H), 2.74-2.51 (m, 4H), 2.50-2.45 (m, 3H), 2.39-2.30 (m, 1H), 2.23-2.12 (m, 1H), 2.10-1.73 (m, 6H), 1.60-1.49 (m, 3H), 1.04 (d, J=6.4 Hz, 3H), 0.92-0.84 (m, 3H).

### Exemplary Synthesis of tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate

### Step 1: Preparation of tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate

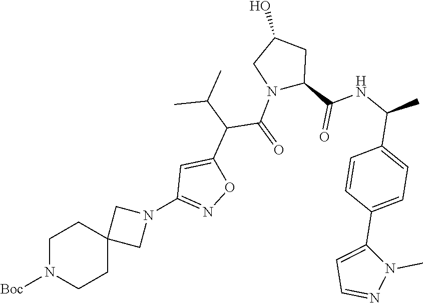 [SMILES: CC(C)C(C(=O)N1C[C@H](O)C[C@H]1C(=O)N[C@@H](C)c1ccc(-c2ccnn2C)cc1)c1cc(N2CC3(CCN(C(=O)OC(C)(C)C)CC3)C2)no1]

To a solution of 2-[3-(7-tert-butoxycarbonyl-2,7-diazaspiro[3.5]nonan-2-yl)isoxazol-5-yl]-3-methyl-butanoic acid (1.5 g, 3.81 mmol, 1.0 eq) and (2S,4R)-4-hydroxy-N-[(1S)-1-[4-(2-methylpyrazol-3-yl)phenyl]ethyl]pyrrolidine-2-carboxamide (1.6 g, 4.57 mmol, 1.2 eq, HCl salt) in CH₂Cl₂ (30 mL) were added diisopropylethylamine (2.0 g, 15.25 mmol, 2.7 mL, 4.0 eq) and HATU (2.9 g, 7.62 mmol, 2.0 eq), and the reaction mixture was stirred at 20° C. for 2 hours. The mixture was concentrated, and the resulting residue was purified by flash chromatography on SiO₂ (gradient: 0-65% THF in petroleum ether) to afford tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (2.2 g, 3.03 mmol, 79% yield) as an off-white solid. LC/MS (ESI) m/z: 690.4 [M+H]⁺.

### Step 2: Chiral Separation of tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate

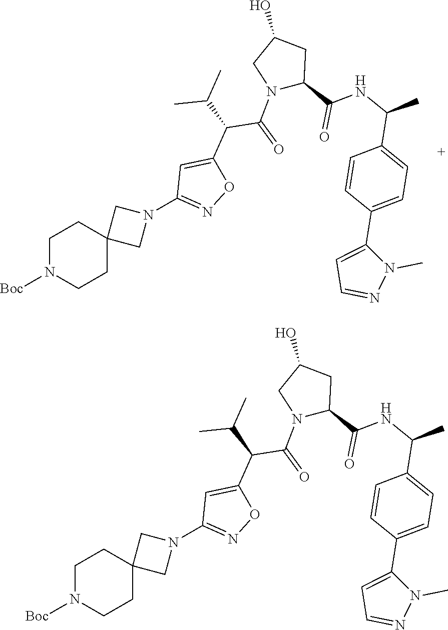 [SMILES: CC(C)[C@H](C(=O)N1C[C@H](O)C[C@H]1C(=O)N[C@@H](C)c1ccc(-c2ccnn2C)cc1)c1cc(N2CC3(CCN(C(=O)OC(C)(C)C)CC3)C2)no1 ; CC(C)[C@@H](C(=O)N1C[C@H](O)C[C@H]1C(=O)N[C@@H](C)c1ccc(-c2ccnn2C)cc1)c1cc(N2CC3(CCN(C(=O)OC(C)(C)C)CC3)C2)no1]

tert-Butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (2.3 g, 3.33 mmol, 1.0 eq) was purified by SFC (40% EtOH in water (0.1% NH₄OH) to obtain the tert-butyl 2-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (1.1 g, 1.56 mmol, 46.87% yield) as a white solid (LC/MS (ESI) m/z: 690.5 [M+H]⁺) and tert-butyl 2-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (830 mg, 1.18 mmol, 35% yield) as a white solid (LC/MS (ESI) m/z: 690.5 [M+H]⁺).

### Exemplary Synthesis of (2S,4R)-1-[(2R)-2-[3-[7-[2-[6-chloro-4-[(3S)-3-(cyano methyl)-4-prop-2-enoyl-piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

(2S,4R)-1-[(2R)-2-[3-[7-[2-[6-chloro-4-[(3S)-3-(cyanomethyl)-4-prop-2-enoyl-piperazin-1- yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide was prepared in an analogous manner to (2S,4R)-1-[(2R)-2-[3-[7-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 2-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate.

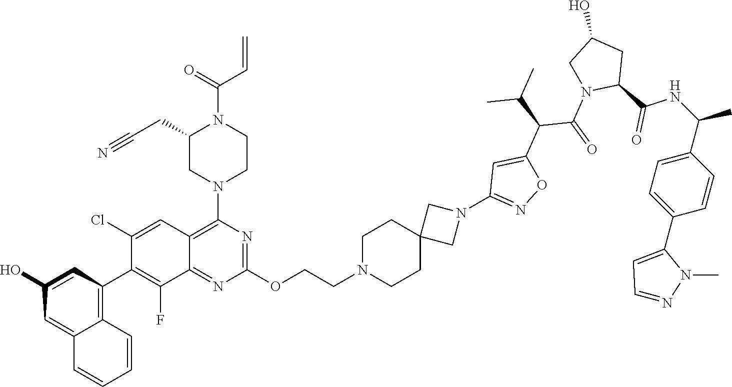

(formic acid salt, white solid). LC/MS (ESI) m/z: 1133.5 [M+H]⁺. ¹H-NMR (400 MHz, DMSO-d₆) δ 8.41 (d, J=7.6 Hz, 1H), 8.15 (s, 1H), 8.11 (s, 1H), 7.81 (d, J=7.6 Hz, 1H), 7.50-7.46 (m, 4H), 7.45-7.44 (m, 2H), 7.39 (s, 1H), 7.37-7.23 (m, 2H), 7.07 (s, 1H), 6.37 (s, 1H), 6.21 (d, J=15.6 Hz, 1H), 5.83-5.81 (m, 1H), 5.10 (s, 1H), 4.99-4.91 (m, 1H), 4.48-4.45 (m, 1H), 4.37 (t, J=7.6 Hz 2H), 4.34-4.26 (m, 3H), 4.25-4.22 (m, 2H), 3.86-3.85 (m, 1H), 3.84 (s, 3H), 3.58-3.53 (m, 5H), 2.69-2.66 (m, 3H), 2.42-2.41 (m, 4H), 2.33 (s, 1H), 2.31 (m, 2H), 2.02-1.99 (m, 1H), 1.77-1.71 (m, 6H), 1.44 (d, J=7.2 Hz, 1H), 1.38 (d, J=7.2 Hz, 3H), 0.96-0.93 (m, 4H), 0.82-0.77 (m, 4H).

### Exemplary Synthesis of (2S,4R)-1-[(2R)-2-[3-[7-[2-[6-chloro-4-[(3S)-3-(cyano methyl)-4-(2-fluoroprop-2-enoyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

(2S,4R)-1-[(2R)-2-[3-[7-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide was prepared in an analogous manner to (2S,4R)-1-[(2R)-2-[3-[7-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 2-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate.

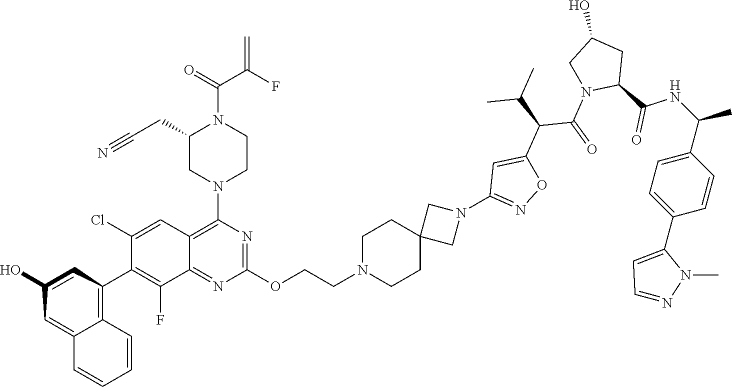

(formic acid salt, yellow solid). LC/MS (ESI) m/z: 1151.3 [M+H]⁺. ¹H-NMR (400 MHz, CD₃OD) δ 8.10 (s, 1H), 7.49-7.48 (m, 1H), 7.45 (s, 1H), 7.43-7.40 (m, 5H), 7.28 (s, 1H), 7.27-7.22 (m, 2H), 7.03 (s, 1H), 6.34 (d, J=2.0 Hz, 1H), 5.88 (s, 1H), 5.37-5.32 (m, 2H), 5.05-4.85 (m, 2H), 4.60-4.44 (m, 6H), 3.85-3.82 (m, 5H), 3.741 (m, 6H), 3.65-3.63 (m, 2H), 3.62-3.61 (m 3H), 3.31-3.10 (m, 1H), 3.08 (d, J=3.6 Hz, 3H), 2.37-2.34 (m, 1H), 2.07-2.04 (m, 1H), 2.02-1.99 (m, 4H), 1.97-1.95 (m, 1H), 1.59-1.51 (m, 3H), 1.32-1.30 (m, 2H), 1.05 (d, J=7.6 Hz, 3H), 0.91-0.87 (m, 3H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(5-methyl-1H-indazol-4-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl (2S)-4-[6-chloro-2-(2,2-dimethoxyethoxy)-8-fluoro-7-(5-methyl-1H-indazol-4-yl)quinazolin-4-yl]-2-(cyanomethyl)piperazine-1-carboxylate

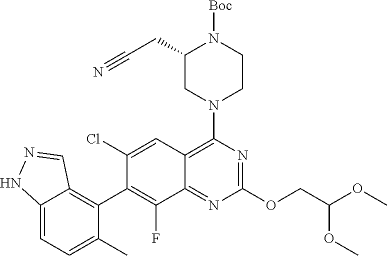 [SMILES: COC(COc1nc(N2CCN(C(=O)OC(C)(C)C)[C@@H](CC#N)C2)c2cc(Cl)c(-c3c(C)ccc4[nH]ncc34)c(F)c2n1)OC]

To a mixture of tert-butyl (2S)-4-[7-bromo-6-chloro-2-(2,2-dimethoxyethoxy)-8-fluoro-quinazolin-4-yl]-2-(cyanomethyl)piperazine-1-carboxylate (700 mg, 1.19 mmol, 1.0 eq), (5-methyl-1H-indazol-4-yl)boronic acid (1.04 g, 5.95 mmol, 5.0 eq), and K₃PO₄ (1.5 M, 3.16 mL, 4.0 eq) in 1-methoxy-2-(2-methoxyethoxy)ethane (14 mL) was added ditertbutyl(cyclopentyl)phosphane; dichloropalladium; iron (117 mg, 0.18 mmol, 0.15 eq) under N₂, and the reaction mixture was stirred at 130° C. for 1 hour under microwave conditions. The reaction mixture was diluted with EtOAc (50 mL), and the resulting organic mixture was washed with water (5×20 mL) followed by brine (3×20 mL), dried over Na₂SO₄, filtered, and concentrated. The resulting residue was purified by flash silica gel chromatography (gradient: 0-40% EtOAc in petroleum ether) to afford tert-butyl (2S)-4-[6-chloro-2-(2,2-dimethoxyethoxy)-8-fluoro-7-(5-methyl-1H-indazol-4-yl)quinazolin-4-yl]-2-(cyanomethyl)piperazine-1-carboxylate (521 mg, 0.63 mmol, 77% purity) as a yellow oil. LC/MS (ESI) m/z: 640.3 [M+H]⁺.

### Step 2: Preparation of 2-[(2S)-4-[6-chloro-8-fluoro-7-(5-methyl-1H-indazol-4-yl)-2-(2-oxoethoxy)quinazolin-4-yl]piperazin-2-yl]acetonitrile

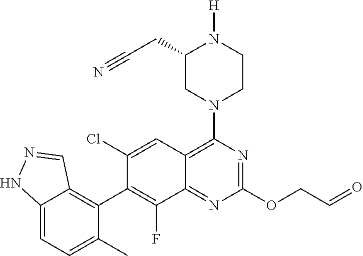 [SMILES: Cc1ccc2[nH]ncc2c1-c1c(Cl)cc2c(N3CCN[C@@H](CC#N)C3)nc(OCC=O)nc2c1F]

To tert-butyl (2S)-4-[6-chloro-2-(2,2-dimethoxyethoxy)-8-fluoro-7-(5-methyl-1H-indazol-4-yl)quinazolin-4-yl]-2-(cyanomethyl)piperazine-1-carboxylate (369 mg, 0.44 mmol, 77% purity, 1.0 eq) in acetone (0.78 mL) was added HCl (781 uL, 8.09 mmol, 37% purity, 18.23 eq), and the reaction mixture was stirred at 25° C. for 20 minutes. The mixture was poured onto saturated aqueous NaHCO₃ (15 mL), and the resulting precipitate was filtered. This material was dissolved in THF (30 mL), and the resulting organic solution was dried over Na₂SO₄, filtered, and dried under reduced pressure to afford 2-[(2S)-4-[6-chloro-8-fluoro-7-(5-methyl-1H-indazol-4-yl)-2-(2-oxoethoxy)quinazolin-4-yl]piperazin-2-yl]acetonitrile (219.24 mg, crude) as a yellow solid. which was used for next step without further purification. LC/MS (ESI) m/z: 494.2 [M+H]⁺.

### Step 3: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(5-methyl-1H-indazol-4-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-chloro-4-[(3S)-3-(cyanomethyl)-4-prop-2-enoyl-piperazin-1-yl]-8-fluoro-7-(5-methyl-1H-indazol-4-yl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl- piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[(2S)-4-[6-chloro-8-fluoro-7-(5-methyl-1H-indazol-4-yl)-2-(2-oxoethoxy)quinazolin-4-yl]piperazin-2-yl]acetonitrile

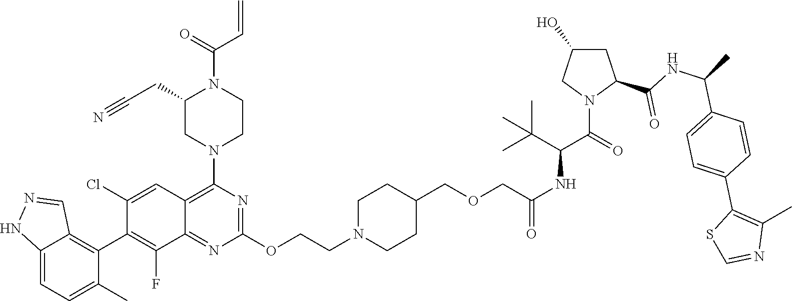 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(C)ccc5[nH]ncc45)c(Cl)cc23)C[C@@H]1CC#N]

(formic acid salt, yellow solid). LC/MS (ESI) m/z: 1131.7 [M+H]⁺. ¹H-NMR (400 MHz, DMSO-d₆) δ 13.18 (s, 1H), 8.98 (s, 1H), 8.45 (d, J=7.6 Hz, 1H), 8.12 (s, 1H), 7.61-7.49 (m, 2H), 7.45-7.32 (m, 5H), 7.28 (d, J=9.6 Hz, 1H), 6.95-6.77 (m, 1H), 6.21 (d, J=18.0 Hz, 1H), 5.79 (d, J=8.8 Hz, 1H), 5.28-4.80 (m, 3H), 4.53 (d, J=9.6 Hz, 1H), 4.50-4.41 (m, 3H), 4.40-4.15 (m, 4H), 4.07 (s, 1H), 3.90 (s, 2H), 3.58-3.55 (m, 1H), 3.41-3.30 (m, 5H), 3.05-2.89 (m, 4H), 2.71 (t, J=5.6 Hz, 2H), 2.45 (s, 3H), 2.16 (d, J=3.8 Hz, 3H), 2.10-1.98 (m, 3H), 1.78-1.71 (m, 1H), 1.69-1.52 (m, 3H), 1.34 (dd, J=2.0, 6.8 Hz, 3H), 1.26-1.13 (m, 2H), 0.92 (s, 9H).

### Exemplary Synthesis of (2S,4R)-1-[(2S)-2-[[2-[[1-[2-[7-(3-amino-1-isoquinolyl)-6-chloro-4-[(3S)-3-(cyanomethyl)-4-prop-2-enoyl-piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl (2S)-4-[7-bromo-6-chloro-2-[2-[4-[(2-ethoxy-2-oxo-ethoxy)methyl]-1-piperidyl]ethoxy]-8-fluoro-quinazolin-4-yl]-2-(cyanomethyl)piperazine-1-carboxylate

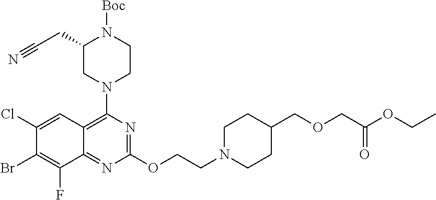 [SMILES: CCOC(=O)COCC1CCN(CCOc2nc(N3CCN(C(=O)OC(C)(C)C)[C@@H](CC#N)C3)c3cc(Cl)c(Br)c(F)c3n2)CC1]

To tert-butyl (2S)-4-(7-bromo-2,6-dichloro-8-fluoro-quinazolin-4-yl)-2-(cyanomethyl)piperazine-1-carboxylate (1.0 g, 1.93 mmol, 1.0 eq) in CH₃CN (10 mL) were added ethyl 2-[[1-(2-hydroxyethyl)-4-piperidyl]methoxy]acetate (614.24 mg, 2.50 mmol, 1.3 eq), Cs₂CO₃ (815.81 mg, 2.50 mmol, 1.3 eq), and DABCO (21.60 mg, 192.61 umol, 21.18 uL, 0.1 eq) at 20° C., and the reaction mixture was stirred at 50° C. for 3 hours. The reaction mixture was filtered, and the filtrate was concentrated under reduced pressure. The resulting residue was purified by flash silica gel chromatography (gradient: 0-4% EtOH in CH₂Cl₂) to afford tert-butyl (2S)-4-[7-bromo-6-chloro-2-[2-[4-[(2-ethoxy-2-oxo-ethoxy)methyl]-1-piperidyl]ethoxy]-8-fluoro-quinazolin-4-yl]-2-(cyanomethyl)piperazine-1-carboxylate (808 mg, 1.04 mmol, 53.9% yield) as a yellow solid. LC/MS (ESI) m/z: 729.2 [M+H]⁺.

### Step 2: Preparation of 2-[[1-[2-[7-(3-amino-1-isoquinolyl)-4-[(3S)-4-tert-butoxycarbonyl-3-(cyanomethyl)piperazin-1-yl]-6-chloro-8-fluoro-quinazolin-2-yl]oxyethyl]-4-piperidyl]methoxy]acetic Acid

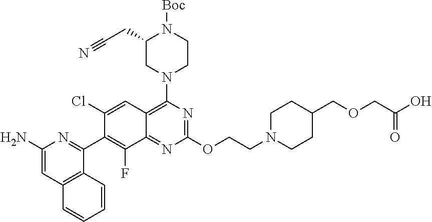 [SMILES: CC(C)(C)OC(=O)N1CCN(c2nc(OCCN3CCC(COCC(=O)O)CC3)nc3c(F)c(-c4nc(N)cc5ccccc45)c(Cl)cc23)C[C@@H]1CC#N]

To a mixture of tert-butyl (2S)-4-[7-bromo-6-chloro-2-[2-[4-[(2-ethoxy-2-oxo-ethoxy)methyl]-1-piperidyl]ethoxy]-8-fluoro-quinazolin-4-yl]-2-(cyanomethyl)piperazine-1-carboxylate (680 mg, 934 umol, 1.0 eq), 1-tributylstannylisoquinolin-3-amine (809.25 mg, 1.87 mmol, 2.0 eq), and LiCl (98.98 mg, 2.34 mmol, 2.5 eq) in dioxane (7 mL) were added CuI (53.36 mg, 280.20 umol, 0.3 eq) and Pd(PPh₃)₄ (215.86 mg, 186.80 umol, 0.2 eq), and the reaction mixture was stirred at 110° C. for 15 hours under N₂. The reaction mixture was filtered, and the solid in the filter was washed with EtOAc (50 mL). The filtrate was concentrated, and the resulting crude product was purified by pre-HPLC (15-60% CH₃CN in water (0.05% HCl)) to afford 2-[[1-[2-[7-(3-amino-1-isoquinolyl)-4-[(3S)-4-tert-butoxycarbonyl-3-(cyanomethyl)piperazin-1-yl]-6-chloro-8-fluoro-quinazolin-2-yl]oxyethyl]-4-piperidyl]methoxy]acetic acid (245 mg, 305.46 umol, 33% yield) as a yellow solid. LC/MS (ESI) m/z: 763.3 [M+H]⁺.

### Step 3: Preparation of tert-butyl (2S)-4-[7-(3-amino-1-isoquinolyl)-6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]quinazolin-4-yl]-2-(cyanomethyl)piperazine-1-carboxylate

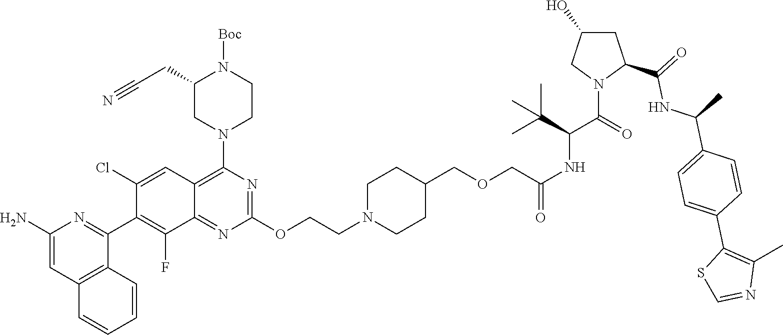 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCN(C(=O)OC(C)(C)C)[C@@H](CC#N)C4)c4cc(Cl)c(-c5nc(N)cc6ccccc56)c(F)c4n3)CC2)C(C)(C)C)cc1]

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (122.9 mg, 0.255 mmol, 1.0 eq, HCl salt) and 2-[[1-[2-[7-(3-amino-1-isoquinolyl)-4-[(3S)-4-tert-butoxycarbonyl-3-(cyanomethyl)piperazin-1-yl]-6-chloro-8-fluoro-quinazolin-2-yl]oxyethyl]-4-piperidyl]methoxy]acetic acid (195 mg, 0.255 mmol, 1.0 eq) in DMF (6 mL) were added diisopropylethylamine (198.1 mg, 1.53 mmol, 6.0 eq) and HATU (97.1 mg, 0.255 mmol, 1.0 eq), and the reaction mixture was stirred at 20° C. for 2 hours. The mixture was diluted with EtOAc (100 mL), and the resulting organic mixture was washed with brine (4×30 mL), dried over anhydrous sodium sulfate, filtered, and concentrated to give tert-butyl(2S)-4-[7-(3-amino-1-isoquinolyl)-6-chloro-8-fluoro-2-[2-[4-[[2-[[(1S)-1-[(2S,4R)-4-hydroxy-2-[[(1S)-1-[4- (4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]quinazolin-4-yl]-2-(cyanomethyl)piperazine-1-carboxylate (380 mg, crude) as a yellow solid. LC/MS (ESI) m/z: 595.6 [M/2+H]⁺.

### Step 4: Preparation of (2S,4R)-1-[(2S)-2-[[2-[[1-[2-[7-(3-amino-1-isoquinolyl)-6-chloro-4-[(3S)-3-(cyanomethyl)piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

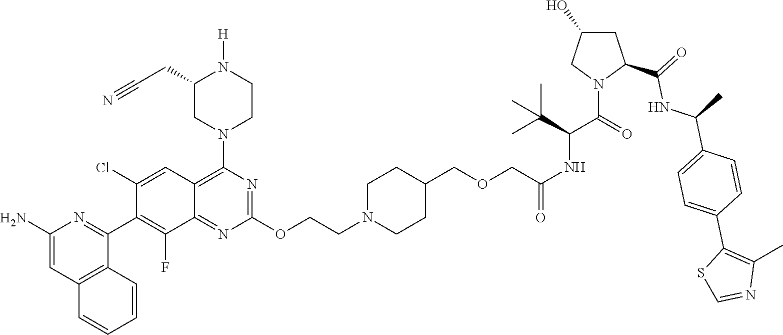 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCN[C@@H](CC#N)C4)c4cc(Cl)c(-c5nc(N)cc6ccccc56)c(F)c4n3)CC2)C(C)(C)C)cc1]

To a solution of tert-butyl (2S)-4-[7-(3-amino-1-isoquinolyl)-6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]quinazolin-4-yl]-2-(cyanomethyl)piperazine-1-carboxylate (350 mg, 0.294 mmol, 1.0 eq) in CH₂Cl₂ (2 mL) was added TFA (637.3 mg, 5.59 mmol, 19.0 eq), and the reaction mixture was stirred at 20° C. for 2 hours. The reaction was concentrated, and the resulting material was made basic by addition of Et₃N. The residue was purified by prep-HPLC (30-60% CH₃CN in water (NH₄HCO₃)) to afford (2S,4R)-1-[(2S)-2-[[2-[[1-[2-[7-(3-amino-1-isoquinolyl)-6-chloro-4-[(3S)-3-(cyanomethyl)piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (100 mg, 84.43 umol, 29% yield) as a yellow solid. LC/MS (ESI) m/z: 1089.5 [M+H]⁺.

### Step 5: Preparation of (2S,4R)-1-[(2S)-2-[[2-[[1-[2-[7-(3-amino-1-isoquinolyl)-6-chloro-4-[(3S)-3-(cyanomethyl)-4-prop-2-enoyl-piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

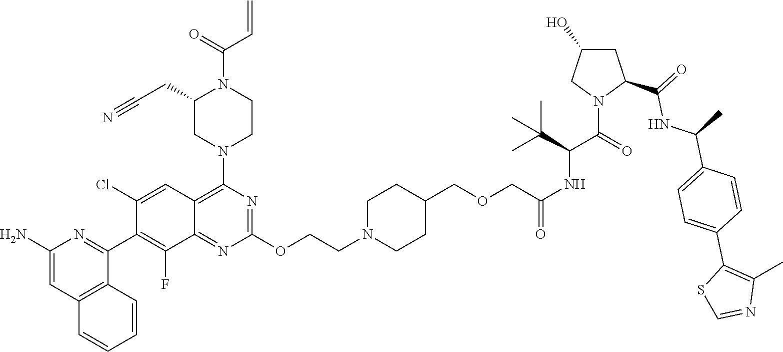 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4nc(N)cc5ccccc45)c(Cl)cc23)C[C@@H]1CC#N]

To a solution of (2S,4R)-1-[(2S)-2-[[2-[[1-[2-[7-(3-amino-1-isoquinolyl)-6-chloro-4-[(3S)-3-(cyanomethyl)piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (100 mg, 0.092 mmol, 1.0 eq) and 2,6-lutidine (39.3 mg, 0.367 mmol, 4.0 eq) in CH₂Cl₂ (5 mL) at −78° C. was added prop-2-enoyl chloride (8.3 mg, 0.092 mmol, 1.0 eq) dropwise, and the reaction mixture was stirred at −78° C. for 0.5 hour. Water (0.2 mL) was then added, and the resulting mixture was stirred for 10 minutes until the temperature reached 20° C. The mixture was concentrated, and the resulting residue was purified by prep-HPLC (10-50% CH₃CN in water (0.225% formic acid)) to afford (2S,4R)-1-[(2S)-2-[[2-[[1-[2-[7-(3-amino-1-isoquinolyl)-6-chloro-4-[(3S)-3-(cyanomethyl)- 4-prop-2-enoyl-piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (27.8 mg, 25% yield, formic Acid salt) as a yellow solid. LC/MS (ESI) m/z: 1143.8 [M+H]⁺. ¹H-NMR (400 MHz, CD₃OD) δ 8.87 (s, 1H), 8.49 (br s, 1H), 8.10 (s, 1H), 7.75-7.59 (m, 1H), 7.57-7.23 (m, 6H), 7.19-7.07 (m, 1H), 7.02-6.90 (m, 1H), 6.88-6.73 (m, 1H), 6.42-6.19 (m, 1H), 5.97-5.76 (m, 1H), 5.18-4.95 (m, 3H), 4.72-4.38 (m, 7H), 4.27-4.09 (m, 1H), 4.06-3.68 (m, 6H), 3.64-3.39 (m, 6H), 3.13-2.96 (m, 2H), 2.94-2.66 (m, 2H), 2.47 (s, 3H), 2.29-2.14 (m, 1H), 2.10-1.80 (m, 4H), 1.70-1.37 (m, 5H), 1.03 (s, 9H).

### Exemplary Synthesis of (2S,4R)-1-[(2S)-2-[[2-[[1-[2-[7-[6-amino-4-methyl-3-(trifluoromethyl)-2-pyridyl]-6-chloro-4-[(3S)-3-(cyanomethyl)-4-prop-2-enoyl-piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 6-bromo-N,N-bis[(4-methoxyphenyl)methyl]-4-methyl-pyridin-2-amine

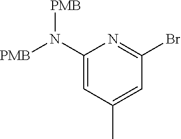

To a solution of 6-bromo-4-methyl-pyridin-2-amine (7.00 g, 37.4 mmol, 1 eq) in DMF (70 mL) at 0° C. was added NaH (5.24 g, 131 mmol, 60% in mineral oil, 3.5 eq) slowly under N₂, and the reaction mixture was stirred at 20° C. for 1 hour. PMB-C1 (13.5 g, 86.1 mmol, 11.72 mL, 2.3 eq) was then added dropwise at 0° C., and the reaction mixture was stirred at 20° C. for 16 hours. Saturated aqueous NH₄Cl (80 mL) was then added at 0° C., and the resulting mixture was extracted with EtOAc (3×100 mL). The combined organic extract was washed with brine (5×60 mL), dried over anhydrous sodium sulfate, filtered, and concentrated. The resulting residue was purified by flash chromatography on SiO₂ (gradient: 0-5% EtOAc in petroleum ether) twice to give 6-bromo-N,N-bis[(4-methoxyphenyl)methyl]-4-methyl-pyridin-2-amine (12.2 g, 27.1 mmol, 72% yield) as a white solid. LC/MS (ESI) m/z: 426.8, 428.8 [M+H]⁺. ¹H-NMR (400 MHz, CDCl₃) δ 7.20-7.12 (m, 4H), 6.90-6.82 (m, 4H), 6.59 (s, 1H), 6.16 (s, 1H), 4.64 (s, 4H), 3.80 (s, 6H), 2.13 (s, 3H).

### Step 2: Preparation of N,N-bis[(4-methoxyphenyl)methyl]-4-methyl-6-tributylstannyl-pyridin-2-amine

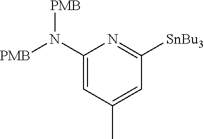

To a solution of 6-bromo-N,N-bis[(4-methoxyphenyl)methyl]-4-methyl-pyridin-2-amine (6.17 g, 14.4 mmol, 1 eq) and tributyl(tributylstannyl)stannane (25.1 g, 43.3 mmol, 21.7 mL, 3 eq) in dioxane (40 mL) were added Pd₂(dba)₃ (1.32 g, 1.44 mmol, 0.1 eq), P(Cy)₃ (810 mg, 2.89 mmol, 0.2 eq), and LiCl (3.06 g, 72.2 mmol, 5 eq), and the reaction mixture was stirred at 110° C. for 16 hours under N₂. The reaction mixture was filtered, and the filter cake was washed with EtOAc (3×50 mL). The filtrate was concentrated under reduced pressure, and the resulting residue was purified by flash chromatography on SiO₂ (gradient: 0-4% THF in petroleum ether) to afford N,N-bis[(4-methoxyphenyl)methyl]-4-methyl-6-tributylstannyl-pyridin-2-amine (6.28 g, 8.77 mmol, 61% yield) as a yellow oil. LC/MS (ESI) m/z: 639.4 [M+H]⁺.

### Step 3: Preparation of tert-butyl(2S)-4-(7-bromo-6-chloro-2,8-difluoro-quinazolin-4-yl)-2-(cyanomethyl)piperazine-1-carboxylate

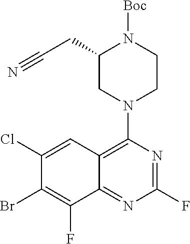 [SMILES: CC(C)(C)OC(=O)N1CCN(c2nc(F)nc3c(F)c(Br)c(Cl)cc23)C[C@@H]1CC#N]

To a solution of tert-butyl (2S)-4-(7-bromo-2,6-dichloro-8-fluoro-quinazolin-4-yl)-2-(cyanomethyl)piperazine-1-carboxylate (5.62 g, 10.8 mmol, 1 eq) in DMA (75 mL) was added KF (25.2 g, 433 mmol, 40 eq), and the reaction mixture was stirred at 120° C. for 21 hours under N₂. The mixture was diluted with EtOAc (400 mL) and filtered, and the filtrate was washed with brine (5×60 mL), dried over anhydrous sodium sulfate, filtered, and concentrated. The resulting residue was purified by flash chromatography on SiO₂ (gradient: 10-20% EtOAc in petroleum ether) followed by flash chromatography on SiO₂ (gradient: 10-60% CH₂Cl₂ in petroleum ether) to give tert-butyl(2S)-4-(7-bromo-6-chloro-2,8-difluoro-quinazolin-4-yl)-2-(cyanomethyl)piperazine-1-carboxylate (2.46 g, 4.26 mmol, 39% yield) as a yellow solid. LC/MS (ESI) m/z: 504.1 [M+H]⁺.

### Step 4: Preparation of tert-butyl(2S)-4-[7-[6-[bis[(4-methoxyphenyl)methyl]amino]-4-methyl-2-pyridyl]-6-chloro-2,8-difluoro-quinazolin-4-yl]-2-(cyanomethyl)piperazine-1-carboxylate

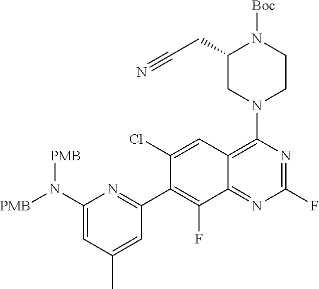

To a mixture of tert-butyl (2S)-4-(7-bromo-6-chloro-2,8-difluoro-quinazolin-4-yl)-2-(cyanomethyl)piperazine-1-carboxylate (1.45 g, 2.88 mmol, 1 eq), N,N-bis[(4-methoxyphenyl)methyl]-4-methyl-6-tributylstannyl-pyridin-2-amine (3.68 g, 5.77 mmol, 2 eq), and LiCl (306 mg, 7.21 mmol, 2.5 eq) in dioxane (25 mL) were added CuI (165 mg, 0.865 mmol, 0.3 eq) and Pd(PPh₃)₄ (667 mg, 0.577 mmol, 0.2 eq), and the reaction mixture was stirred at 110° C. for 16 hours under N₂. The reaction mixture was filtered, and the filtrate was concentrated. The resulting residue was purified by flash chromatography on SiO₂ (gradient: 5-20% THF in petroleum ether) to afford tert-butyl(2S)-4-[7-[6-[bis[(4-methoxyphenyl)methyl]amino]-4-methyl-2-pyridyl]-6-chloro-2,8-difluoro-quinazolin-4-yl]-2-(cyanomethyl)piperazine-1-carboxylate (1.60 g, 2.04 mmol, 71% yield) as a yellow solid. LC/MS (ESI) m/z: 770.3 [M+H]⁺.

### Step 5: Preparation of tert-butyl(2S)-4-[7-[6-[bis[(4-methoxyphenyl)methyl]amino]-3-iodo-4-methyl-2-pyridyl]-6-chloro-2,8-difluoro-quinazolin-4-yl]-2-(cyanomethyl)piperazine-1-carboxylate

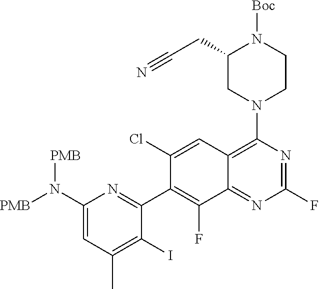

To a solution of tert-butyl (2S)-4-[7-[6-[bis[(4-methoxyphenyl)methyl]amino]-4-methyl-2-pyridyl]-6-chloro-2,8-difluoro-quinazolin-4-yl]-2-(cyanomethyl)piperazine-1-carboxylate (1.60 g, 2.08 mmol, 1 eq) in DMF (15 mL) were added NIS (2.34 g, 10.4 mmol, 5 eq) and TsOH (17.9 mg, 0.104 mmol, 0.05 eq), and the reaction mixture was stirred at 25° C. for 16 hours under N₂. EtOAc (250 mL) was then added, and the resulting organic mixture was washed with brine (6×40 mL), dried over anhydrous sodium sulfate, filtered, and concentrated. The resulting residue purified by flash chromatography on SiO₂ (5-20% THF in petroleum ether) to afford tert-butyl(2S)-4-[7-[6-[bis[(4-methoxyphenyl)methyl]amino]-3-iodo-4-methyl-2-pyridyl]-6-chloro-2,8-difluoro-quinazolin-4-yl]-2-(cyanomethyl)piperazine-1-carboxylate (1.60 g, 1.75 mmol, 84% yield) as a yellow solid. LC/MS (ESI) m/z: 448.7 [M/2+H]⁺.

### Step 6: Preparation of tert-butyl(2S)-4-[7-[6-[bis[(4-methoxyphenyl)methyl]amino]-4-methyl-3-(trifluoromethyl)-2-pyridyl]-6-chloro-2,8-difluoro-quinazolin-4-yl]-2-(cyanomethyl)piperazine-1-carboxylate

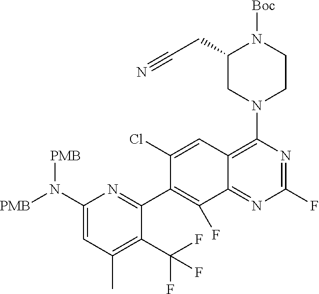

To a solution of tert-butyl (2S)-4-[7-[6-[bis[(4-methoxyphenyl)methyl]amino]-3-iodo-4-methyl-2-pyridyl]-6-chloro-2,8-difluoro-quinazolin-4-yl]-2-(cyanomethyl)piperazine-1-carboxylate (1.60 g, 1.79 mmol, 1 eq) in DMA (50 mL) were added CuI (3.40 g, 17.9 mmol, 10 eq) and methyl 2,2-difluoro-2-fluorosulfonyl-acetate (8.57 g, 44.6 mmol, 5.68 mL, 25 eq), and the reaction mixture was stirred at 90° C. for 16 hours under N₂. The mixture was diluted with EtOAc (250 mL) and filtered, and the filtrate was concentrated under reduced pressure. The resulting residue was purified by flash chromatography on SiO₂ (gradient: 5-20% THF in petroleum ether) to afford tert-butyl(2S)-4-[7-[6-[bis[(4-methoxyphenyl)methyl]amino]-4-methyl-3-(trifluoromethyl)-2-pyridyl]-6-chloro-2,8-difluoro-quinazolin-4-yl]-2-(cyanomethyl)piperazine-1-carboxylate (1.86 g, 82% purity) as a yellow oil. LC/MS (ESI) m/z: 419.8 [M/2+H]⁺.

### Step 7: Preparation of tert-butyl (2S)-4-[7-[6-[bis[(4-methoxyphenyl)methyl]amino]-4-methyl-3-(trifluoromethyl)-2-pyridyl]-2-[2-[4-[(2-tert-butoxy-2-oxo-ethoxy)methyl]-1-piperidyl]ethoxy]-6-chloro-8-fluoro-quinazolin-4-yl]-2-(cyanomethyl)piperazine-1-carboxylate

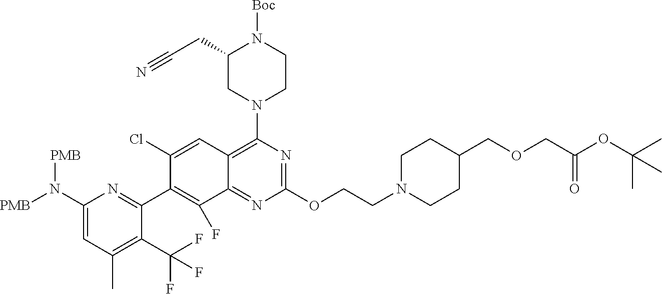

To a mixture of tert-butyl (2S)-4-[7-[6-[bis[(4-methoxyphenyl)methyl]amino]-4-methyl-3-(trifluoromethyl)-2-pyridyl]-6-chloro-2,8-difluoro-quinazolin-4-yl]-2-(cyanomethyl)piperazine-1-carboxylate (940 mg, 1.12 mmol, 1 eq) and tert-butyl 2-[[1-(2-hydroxyethyl)-4-piperidyl]methoxy]acetate (552 mg, 2.02 mmol, 1.8 eq) in DMF (5 mL) and THF (5 mL) were added Cs₂CO₃ (585 mg, 1.79 mmol, 1.6 eq) and DABCO (25 mg, 0.22 mmol, 0.2 eq), and the reaction mixture was stirred at 20° C. for 16 hours. The reaction mixture was diluted with EtOAc (150 mL) and then filtered. The filtrate was washed with brine (4×30 mL), dried over anhydrous sodium sulfate, filtered, and concentrated. The resulting residue was purified by flash chromatography on SiO₂ (gradient: 0-70 EtOAc in petroleum ether) to afford tert-butyl (2S)-4-[7-[6-[bis[(4-methoxyphenyl)methyl]amino]-4-methyl-3-(trifluoromethyl)-2-pyridyl]-2-[2-[4-[(2-tert-butoxy-2-oxo-ethoxy)methyl]-1-piperidyl]ethoxy]-6-chloro-8-fluoro-quinazolin-4-yl]-2-(cyanomethyl)piperazine-1-carboxylate (620 mg, 0.528 mmol, 43% yield) as a yellow solid. LC/MS (ESI) m/z: 1091.6 [M+H]⁺.

### Step 8: Preparation of 2-[[1-[2-[7-[6-[bis[(4-methoxyphenyl)methyl]amino]-4-methyl-3-(trifluoromethyl)-2-pyridyl]-4-[(3S)-4-tert-butoxycarbonyl-3-(cyanomethyl)piperazin-1-yl]-6-chloro-8-fluoro-quinazolin-2-yl]oxyethyl]-4-piperidyl]methoxy]acetic Acid

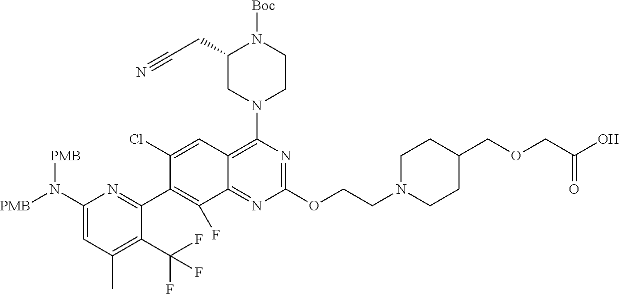

To a solution of tert-butyl (2S)-4-[7-[6-[bis[(4-methoxyphenyl)methyl]amino]-4-methyl-3-(trifluoromethyl)-2-pyridyl]-2-[2-[4-[(2-tert-butoxy-2-oxo-ethoxy)methyl]-1-piperidyl]ethoxy]-6-chloro-8-fluoro-quinazolin-4-yl]-2-(cyanomethyl)piperazine-1-carboxylate (500 mg, 0.458 mmol, 1 eq) in THF (5 mL) and CH₃OH (3 mL) was added a solution of LiOH.H₂O (577 mg, 13.7 mmol, 30 eq) in H₂O (3 mL), and the reaction mixture was stirred at 0° C. for 1 hour, then at 20° C. for 1 hour. The pH of the reaction mixture was adjusted to pH=7 by addition of aqueous HCl (2N), and the resulting mixture was concentrated. The resulting residue was acidified by addition of aqueous HCl (2N) until pH=3-4 and the mixture was lyophilized to afford 2-[[1-[2-[7-[6-[bis[(4-methoxyphenyl)methyl]amino]-4-methyl-3-(trifluoromethyl)-2-pyridyl]-4-[(3S)-4-tert-butoxycarbonyl-3-(cyanomethyl)piperazin-1-yl]-6-chloro-8-fluoro-quinazolin-2-yl]oxyethyl]-4-piperidyl]methoxy]acetic acid (474 mg, crude) as a yellow solid. LC/MS (ESI) m/z: 518.3 [M/2+H]⁺.

### Step 9: Preparation of tert-butyl (2S)-4-[7-[6-[bis[(4-methoxyphenyl)methyl]amino]-4-methyl-3-(trifluoromethyl)-2-pyridyl]-6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]quinazolin-4-yl]-2-(cyanomethyl)piperazine-1-carboxylate

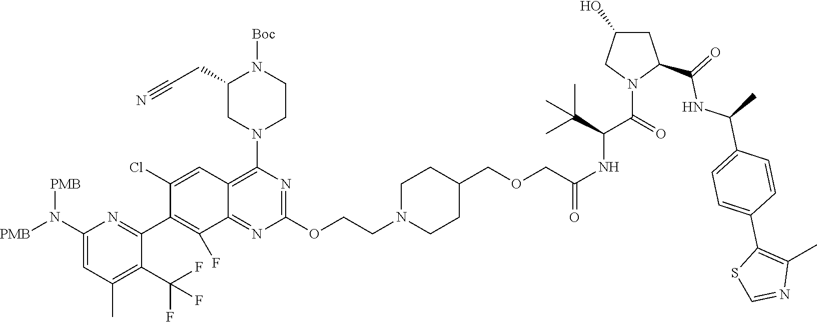

To a mixture of 2-[[1-[2-[7-[6-[bis[(4-methoxyphenyl)methyl]amino]-4-methyl-3-(trifluoromethyl)-2-pyridyl]-4-[(3S)-4-tert-butoxycarbonyl-3-(cyanomethyl)piperazin-1-yl]-6-chloro-8-fluoro-quinazolin-2-yl]oxyethyl]-4-piperidyl]methoxy]acetic acid (420 mg, crude) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (195 mg, 0.406 mmol, 1 eq, HCl salt) in DMF (8 mL) were added diisopropylethylamine (262 mg, 2.03 mmol, 5 eq) and HATU (154 mg, 0.406 mmol, 1 eq), and the reaction mixture was stirred at 20° C. for 2 hours. EtOAc (100 mL) was added, and the resulting organic mixture was washed with brine (4×30 mL), dried over anhydrous sodium sulfate, filtered, and concentrated. The resulting residue was purified by flash chromatography on SiO₂ (gradient: 2-8% CH₃OH in CH₂Cl₂) to afford tert-butyl (2S)-4-[7-[6-[bis[(4-methoxyphenyl)methyl]amino]-4-methyl-3-(trifluoromethyl)-2-pyridyl]-6-chloro-8-fluoro-2-[2-[4-[[2-[[(1S)-1-[(2S ,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]quinazolin-4-yl]-2-(cyanomethyl)piperazine-1-carboxylate (378 mg, 52% yield) as a yellow solid. LC/MS (ESI) m/z: 731.9 [M/2+H]⁺.

### Step 10: Preparation of (2S,4R)-1-[(2S)-2-[[2-[[1-[2-[7-[6-amino-4-methyl-3-(trifluoromethyl)-2-pyridyl]-6-chloro-4-[(3S)-3-(cyanomethyl)piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

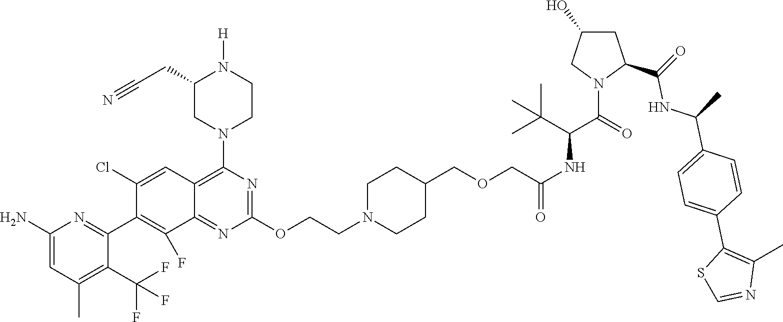 [SMILES: Cc1cc(N)nc(-c2c(Cl)cc3c(N4CCN[C@@H](CC#N)C4)nc(OCCN4CCC(COCC(=O)N[C@H](C(=O)N5C[C@H](O)C[C@H]5C(=O)N[C@@H](C)c5ccc(-c6scnc6C)cc5)C(C)(C)C)CC4)nc3c2F)c1C(F)(F)F]

To a solution of tert-butyl (2S)-4-[7-[6-[bis[(4-methoxyphenyl)methyl]amino]-4-methyl-3-(trifluoromethyl)-2-pyridyl]-6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]quinazolin-4-yl]-2-(cyanomethyl)piperazine-1-carboxylate (225 mg, 0.154 mmol, 1 eq) in TFA (4 mL) at 0° C. was added TfOH (0.2 mL) dropwise, and the reaction mixture was stirred at 0° C. for 30 minutes under N₂. This procedure was repeated 2 additional times following this procedure starting with 50 mg of starting material. The 3 reaction mixtures were combined and concentrated. The resulting residue was treated with saturated aqueous Na₂CO₃ until the pH of the resulting aqueous solution reached pH=9. This aqueous mixture was then extracted with 15:1 CH₂Cl₂/CH₃OH (3×40 mL), and the combined organic extract was washed with brine (30 mL), dried over anhydrous sodium sulfate, filtered, and concentrated. The resulting crude product was purified by prep-HPLC (30-80% CH₃CN in water (10 mM NH₄HCO₃)) to afford (2S,4R)-1-[(2S)-2-[[2-[[1-[2-[7-[6-amino-4-methyl-3-(trifluoromethyl)-2-pyridyl]-6-chloro-4-[(3S)-3-(cyanomethyl)piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (160 mg) as a white solid. LC/MS (ESI) m/z: 561.1 [M/2+H]⁺.

### Step 11: Preparation of (2S,4R)-1-[(2S)-2-[[2-[[1-[2-[7-[6-amino-4-methyl-3-(trifluoromethyl)-2-pyridyl]-6-chloro-4-[(3S)-3-(cyanomethyl)-4-prop-2-enoyl-piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

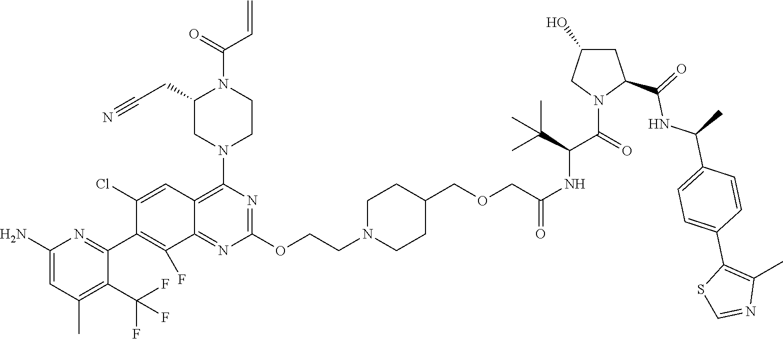 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4nc(N)cc(C)c4C(F)(F)F)c(Cl)cc23)C[C@@H]1CC#N]

To a mixture of (2S,4R)-1-[(2S)-2-[[2-[[1-[2-[7-[6-amino-4-methyl-3-(trifluoromethyl)-2-pyridyl]-6-chloro-4-[(3S)-3-(cyanomethyl)piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (160 mg, 0.143 mmol, 1 eq), diidopropylamine (37 mg, 0.29 mmol, 2 eq), and 2,6-lutidine (31 mg, 0.29 mmol, 2 eq) in CH₂Cl₂ (5 mL) at −78° C. was added prop-2-enoyl chloride (12.9 mg, 0.143 mmol, 1 eq) dropwise, and the reaction mixture was stirred at −78° C. under N₂ for 0.5 hour. Water (0.2 mL) was then added, and the resulting mixture was stirred for 10 minutes until it warmed to 20° C. The mixture was concentrated under reduced pressure, and the resulting residue was purified by prep-HPLC (10-50% CH₃CN in water (0.225% formic acid)) to afford (2S,4R)-1-[(2S)-2-[[2-[[1-[2-[7-[6-amino-4-methyl-3-(trifluoromethyl)-2-pyridyl]-6-chloro-4-[(3S)-3-(cyanomethyl)-4-prop-2-enoyl-piperazin-1-yl]-8-fluoro-quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (55.7 mg, 0.047 mmol, 33% yield, formic acid salt) as a white solid. LC/MS (ESI) m/z: 1175.8 [M+H]⁺. ¹H-NMR (400 MHz, CD₃OD) δ 8.90-8.83 (m, 1H), 8.53 (s, 1H), 7.97 (s, 1H), 7.51-7.33 (m, 4H), 6.99-6.72 (m, 1H), 6.60 (s, 1H), 6.30 (d, J=16.4 Hz, 1H), 5.83 (d, J=10.4 Hz, 1H), 5.14-5.05 (m, 1H), 5.02-4.96 (m, 1H), 4.82-4.66 (m, 4H), 4.61-4.52 (m, 1H), 4.50-4.34 (m, 3H), 4.20-4.08 (m, 1H), 4.06-3.91 (m, 2H), 3.89-3.80 (m, 2H), 3.77-3.64 (m, 2H), 3.53-3.41 (m, 4H), 3.30-3.18 (m, 2H), 3.10-2.96 (m, 2H), 2.83-2.60 (m, 2H), 2.50-2.46 (m, 3H), 2.44 (s, 3H), 2.30-2.15 (m, 1H), 2.00-1.81 (m, 4H), 1.60-1.45 (m, 5H), 1.08-0.98 (m, 9H).

### Exemplary Synthesis of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-[[5-(1-methoxycarbonyl-2-methyl-propyl)isoxazol-3-yl]oxymethyl]-4-methyl-piperidine-1-carboxylate

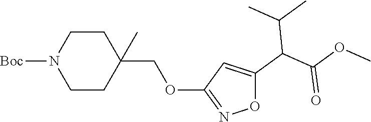 [SMILES: COC(=O)C(c1cc(OCC2(C)CCN(C(=O)OC(C)(C)C)CC2)no1)C(C)C]

To a mixture of tert-butyl 4-(hydroxymethyl)-4-methyl-piperidine-1-carboxylate (2.00 g, 8.72 mmol, 1 eq), methyl 2-(3-hydroxyisoxazol-5-yl)-3-methyl-butanoate (1.74 g, 8.72 mmol, 1 eq), and PPh₃ (3.43 g, 13.1 mmol, 1.5 eq) in THF (20 mL) at 0° C. was added DIAD (2.65 g, 13.1 mmol, 2.54 mL, 1.5 eq), and the reaction mixture was stirred at 60° C. for 12 hours under N₂. The reaction mixture was concentrated, and the resulting residue was purified by flash chromatography on SiO₂ (gradient: 0-3% THF in petroleum ether) to afford tert-butyl 4-[[5-(1-methoxycarbonyl-2-methyl-propyl)isoxazol-3-yl]oxymethyl]-4-methyl-piperidine-1-carboxylate (3.10 g, 7.55 mmol, 87% yield) as a colorless oil. LC/MS (ESI) m/z: 411.3 [M+H]⁺.

### Step 2: Preparation of 2-[3-[(1-tert-butoxycarbonyl-4-methyl-4-piperidyl)methoxy]isoxazol-5-yl]-3-methyl-butanoic Acid

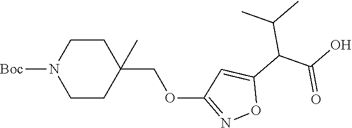 [SMILES: CC(C)C(C(=O)O)c1cc(OCC2(C)CCN(C(=O)OC(C)(C)C)CC2)no1]

To a solution of 2-[3-[(1-tert-butoxycarbonyl-4-methyl-4-piperidyl)methoxy]isoxazol-5-yl]-3-methyl-butanoic acid (3.10 g, 7.55 mmol, 1 eq) in CH₃OH (15 mL) and H₂O (15 mL) was added LiOH.H₂O (1.58 g, 37.76 mmol, 5 eq), and the reaction mixture was stirred at 20° C. for 2 hours under N₂. Aqueous HCl (2N, 2 mL) was then added, and the resulting acidic mixture (pH ˜4 to 5) was lyophilized to give 2-[3-[(1-tert-butoxycarbonyl-4-methyl-4-piperidyl)methoxy]isoxazol-5-yl]-3-methyl-butanoic acid (3.0 g, crude) as a white solid. LC/MS (ESI) m/z: 397.2 [M+H]⁺.

### Step 3: Preparation of tert-butyl 4-[[5-[(1S)-1-[(2S,4R)-4-hydroxy-2-[[(1S)-1-[4-(4-methylthiazol-5-yl) phenyl]ethyl]carbamoyl]pyrrolidine-1-carbonyl]-2-methyl-propyl]isoxazol-3-yl]oxymethyl]-4-methyl-piperidine-1-carboxylate & tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate

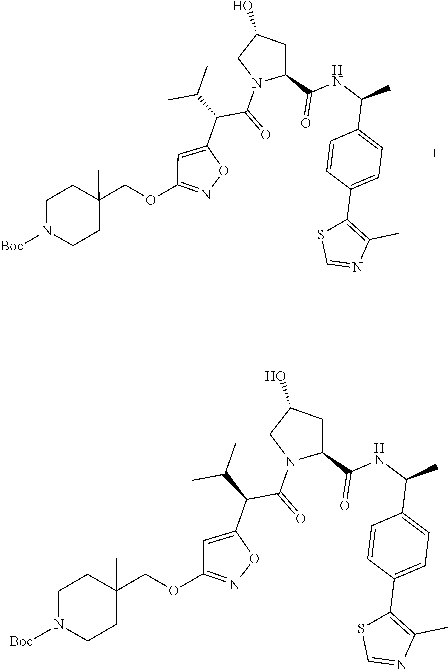 [SMILES: Cc1ncsc1-c1ccc([C@H](C)NC(=O)[C@@H]2C[C@@H](O)CN2C(=O)[C@H](c2cc(OCC3(C)CCN(C(=O)OC(C)(C)C)CC3)no2)C(C)C)cc1 ; Cc1ncsc1-c1ccc([C@H](C)NC(=O)[C@@H]2C[C@@H](O)CN2C(=O)[C@@H](c2cc(OCC3(C)CCN(C(=O)OC(C)(C)C)CC3)no2)C(C)C)cc1]

To a solution of 2-[3-[(1-tert-butoxycarbonyl-4-methyl-4-piperidyl)methoxy]isoxazol-5-yl]-3-methyl-butanoic acid (1.40 g, crude) and diisopropylethylamine (456 mg, 3.53 mmol, 0.615 mL, 1 eq) in CH₂Cl₂ (15 mL) was added HATU (1.34 g, 3.53 mmol, 1 eq), and the reaction mixture was stirred at 20° C. for 0.5 hour. (2S,4R)-4-Hydroxy-N-[(1S)-1-[4-(4-methylthiazol-5-yl)phenyl]ethyl]pyrrolidine-2-carboxamide (1.17 g, 3.53 mmol, 1 eq) was then added, and the reaction mixture was stirred at 20° C. for 2 hours. The reaction mixture was concentrated, and the resulting residue was purified by flash chromatography on SiO₂ (gradient: 0-3% THF in petroleum ether) to give a mixture of the desired product (2.23 g) as a yellow solid. Separation by chiral SFC (0.1% NH₄OH in EtOH) afforded tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (970 mg, 1.34 mmol, 38% yield) as a white solid (LC/MS (ESI) m/z: 710.6 [M+H]⁺) and tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (1.10 g, 1.43 mmol, 40% yield) as a light yellow solid (LC/MS (ESI) m/z: 710.6 [M+H]⁺).

### Step 4: Preparation of (2S,4R)-4-hydroxy-1-[(2R)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide

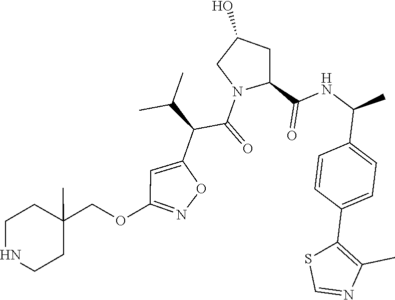 [SMILES: Cc1ncsc1-c1ccc([C@H](C)NC(=O)[C@@H]2C[C@@H](O)CN2C(=O)[C@@H](c2cc(OCC3(C)CCNCC3)no2)C(C)C)cc1]

To a solution of tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (1.10 g, 1.43 mmol, 1 eq) in CH₂Cl₂ (7 mL) was added HCl (4N in EtOAc, 7 mL), and the reaction mixture was stirred at 20° C. for 2 hours. The reaction mixture was adjusted to pH ˜7-8 by adding saturated aqueous NaHCO₃. The aqueous phase was extracted with 1:10 CH₃OH/CH₂Cl₂ (20 mL), and the combined organic extracts was washed with brine (20 mL), dried over Na₂SO₄, filtered, and concentrated to give (2S,4R)-4-hydroxy-1-[(2R)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide (510 mg, crude) as a light yellow solid. which would be directly used in the next step. LC/MS (ESI) m/z: 610.4 [M+H]⁺.

### Step 5: Preparation of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2R)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide.

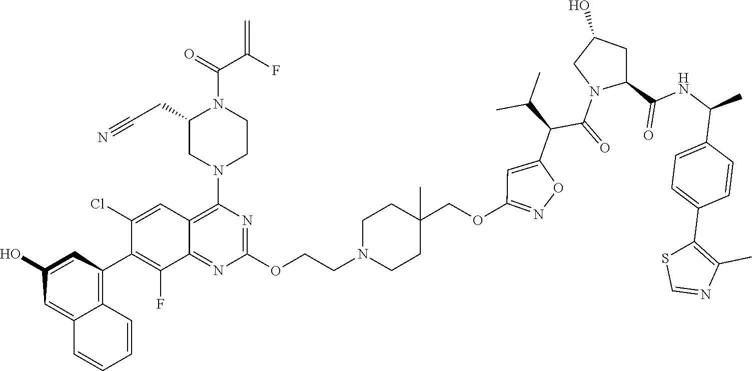

(formic acid salt, white solid). LC/MS (ESI) m/z: 1171.2 [M+H]⁺. ¹H-NMR (400 MHz, CD₃OD) δ 8.89-8.86 (m, 1H), 8.49-8.45 (m, 1H), 8.09 (s, 1H), 7.77-7.72 (m, 1H), 7.47-7.43 (m, 2H), 7.42-7.36 (m, 3H), 7.27 (d, J=2.0 Hz, 1H), 7.23-7.16 (m, 2H), 7.03 (d, J=2.0 Hz, 1H), 6.04-5.93 (m, 1H), 5.45-5.29 (m, 2H), 5.07-4.93 (m, 2H), 4.84-4.71 (m, 3H), 4.59-4.48 (m, 3H), 4.45-4.38 (m, 1H), 4.05-3.98 (m, 2H), 3.83 (dd, J=10.8, 4.0 Hz, 2H), 3.77-3.57 (m, 4H), 3.54-3.36 (m, 3H), 3.29-3.24 (m, 1H), 3.16-3.04 (m, 4H), 2.49-2.45 (m, 3H), 2.40-2.32 (m, 1H), 2.22-2.14 (m, 1H), 2.06-1.95 (m, 1H), 1.94-1.86 (m, 2H), 1.64 (d, J=13.6 Hz, 2H), 1.58 (d, J=7.2 Hz, 3H), 1.15-1.12 (m, 3H), 1.07-1.03 (m, 3H), 0.91-0.86 (m, 3H).

### Exemplary Synthesis of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

### Step 1: Preparation of tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate

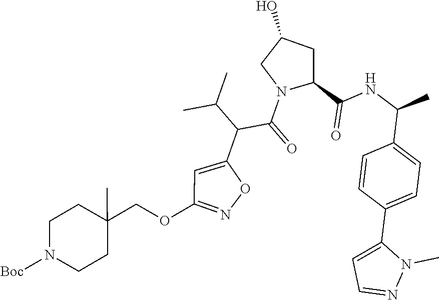 [SMILES: CC(C)C(C(=O)N1C[C@H](O)C[C@H]1C(=O)N[C@@H](C)c1ccc(-c2ccnn2C)cc1)c1cc(OCC2(C)CCN(C(=O)OC(C)(C)C)CC2)no1]

To 2-[3-[(1-tert-butoxycarbonyl-4-methyl-4-piperidyl)methoxy]isoxazol-5-yl]-3-methyl-butanoic acid (1.60 g, 4.04 mmol, 1 eq) in CH₂Cl₂ (40 mL) were added diisopropylethylamine (2.61 g, 20.2 mmol, 3.51 mL, 5 eq) and HATU (1.99 g, 5.25 mmol, 1.3 eq), and the reaction mixture was stirred at 20° C. for 10 minutes. (2S,4R)-4-Hydroxy-N-[(1S)-1-[4-(2-methylpyrazol-3-yl)phenyl]ethyl]pyrrolidine-2-carboxamide (1.42 g, 4.04 mmol, 1 eq, HCl) was then added, and the reaction mixture was stirred at 20° C. for 1 hour. The mixture was poured onto water (30 mL) and the layers were separated. The aqueous layer was extracted with CH₂Cl₂ (30 mL), and the combined organic extract was dried over anhydrous Na₂SO₄, filtered, and concentrated. The resulting crude product was purified by flash silica gel chromatography (gradient: 0-60% THF/petroleum ether) to give tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate (2.20 g, 3.18 mmol, 79% yield) as a yellow gum. LC/MS (ESI) m/z: 693.5 [M+H]⁺.

### Step 2: Chiral separation of tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate

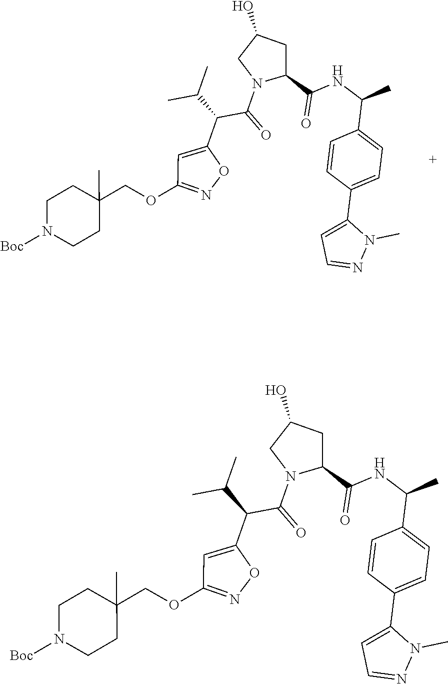 [SMILES: CC(C)[C@H](C(=O)N1C[C@H](O)C[C@H]1C(=O)N[C@@H](C)c1ccc(-c2ccnn2C)cc1)c1cc(OCC2(C)CCN(C(=O)OC(C)(C)C)CC2)no1 ; CC(C)[C@@H](C(=O)N1C[C@H](O)C[C@H]1C(=O)N[C@@H](C)c1ccc(-c2ccnn2C)cc1)c1cc(OCC2(C)CCN(C(=O)OC(C)(C)C)CC2)no1]

tert-Butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate (2.00 g, 2.89 mmol, 1 eq) was separated by SFC (45% EtOH in 0.1% NH₄OH) to give tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate (751 mg, 1.08 mmol, 38% yield) as a white solid (LC/MS (ESI) m/z: 693.6 [M+H]⁺) and tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate (962 mg, 1.39 mmol, 48% yield) as a white solid ((LC/MS (ESI) m/z: 693.6 [M+H]⁺).

### Step 3: Preparation of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

(2S,4R)-1-[(2R)-2-[3-[[1-[2-[6-Chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide starting from tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]-4-methyl-piperidine-1-carboxylate.

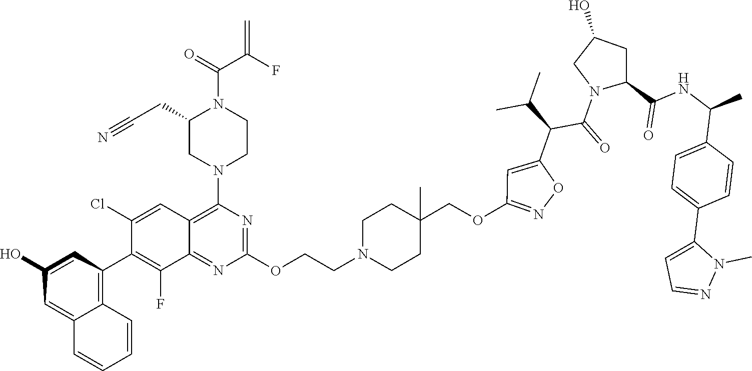

(formic acid salt, white solid). LC/MS (ESI) m/z: 1154.5 [M+H]⁺. ¹H-NMR (400 MHz, CD₃OD) δ 8.52 (s, 1H), 8.07 (s, 1H), 7.75 (d, J=8.4 Hz, 1H), 7.50-7.37 (m, 6H), 7.29-7.14 (m, 3H), 7.05-7.00 (m, 1H), 6.36-6.30 (m, 1H), 6.02-5.93 (m, 1H), 5.46-5.25 (m, 2H), 5.08-4.95 (m, 2H), 4.78-4.70 (m, 2H), 4.57-4.37 (m, 4H), 4.05-3.95 (m, 2H), 3.86-3.45 (m, 9H), 3.26-3.00 (m, 6H), 2.97-2.78 (m, 2H), 2.43-2.14 (m, 2H), 2.06-1.90 (m, 1H), 1.88-1.73 (m, 2H), 1.64-1.48 (m, 5H), 1.33-1.25 (m, 1H), 1.14-1.08 (m, 3H), 1.05 (d, J=6.8 Hz, 3H), 0.92-0.83 (m, 3H).

### Exemplary Synthesis of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-fluoro-4-[[5-(1-methoxycarbonyl-2-methyl-propyl)isoxazol-3-yl]oxymethyl]piperidine-1-carboxylate

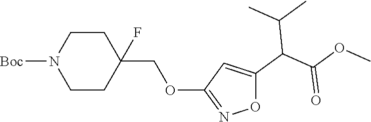 [SMILES: COC(=O)C(c1cc(OCC2(F)CCN(C(=O)OC(C)(C)C)CC2)no1)C(C)C]

To a mixture of tert-butyl 4-fluoro-4-(hydroxymethyl)piperidine-1-carboxylate (2.00 g, 8.57 mmol, 1 eq) and methyl 2-(3-hydroxyisoxazol-5-yl)-3-methyl-butanoate (1.71 g, 8.57 mmol, 1 eq) in toluene (20 mL) at 0° C. were added PPh₃ (3.37 g, 12.9 mmol, 1.5 eq) and DIAD (4.33 g, 21.4 mmol, 4.17 mL, 2.5 eq), and the reaction mixture was stirred under N₂ at 110° C. for 10 hours. The reaction mixture was diluted with water (15 mL) and extracted with CH₂Cl₂ (3×20 mL). The combined organic extract was washed with brine (3×20 mL), dried over Na₂SO₄, filtered, and concentrated. The resulting residue was purified by flash silica gel chromatography (gradient: 0-6% THF/Petroleum ether) to give tert-butyl 4-fluoro-4-[[5-(1-methoxycarbonyl-2-methyl-propyl)isoxazol-3-yl]oxymethyl]piperidine-1-carboxylate (1.15 g, 2.57 mmol, 60% yield) as a colorless oil. LC/MS (ESI) m/z: 415.1 [M+H]⁺.

### Step 2: Preparation of 2-[3-[(1-tert-butoxycarbonyl-4-fluoro-4-piperidyl)methoxy]isoxazol-5-yl]-3-methyl-butanoic Acid

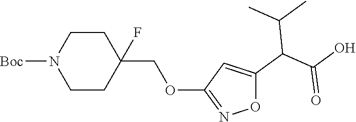 [SMILES: CC(C)C(C(=O)O)c1cc(OCC2(F)CCN(C(=O)OC(C)(C)C)CC2)no1]

To a solution of tert-butyl 4-fluoro-4-[[5-(1-methoxycarbonyl-2-methyl-propyl)isoxazol-3-yl]oxymethyl]piperidine-1-carboxylate (1.87 g, 4.51 mmol, 1 eq) in THF (15 mL), H₂O (15 mL), and CH₃OH (15 mL) was added LiOH.H₂O (946.58 mg, 22.56 mmol, 5 eq), and the reaction mixture was stirred at 25° C. for 1 hour. The reaction was concentrated followed by addition of 2N aqueous HCl to adjust the pH (pH=5). Water (15 mL) was then added, and the resulting aqueous mixture was extracted with CH₂Cl₂ (3×20 mL). The combined organic extract was washed with brine (3×20 mL), dried over Na₂SO₄, filtered, and concentrated to give 2-[3-[(1-tert-butoxycarbonyl-4-fluoro-4-piperidyl)methoxy]isoxazol-5-yl]-3-methyl-butanoic acid (1.8 g, crude) as a yellow oil. LC/MS (ESI) m/z: 401.1 [M+H]⁺.

### Step 3: Preparation of tert-butyl 4-fluoro-4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate & tert-butyl 4-fluoro-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

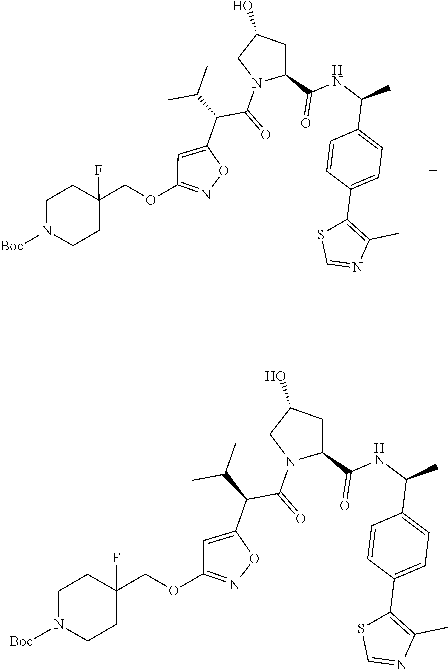 [SMILES: Cc1ncsc1-c1ccc([C@H](C)NC(=O)[C@@H]2C[C@@H](O)CN2C(=O)[C@H](c2cc(OCC3(F)CCN(C(=O)OC(C)(C)C)CC3)no2)C(C)C)cc1 ; Cc1ncsc1-c1ccc([C@H](C)NC(=O)[C@@H]2C[C@@H](O)CN2C(=O)[C@@H](c2cc(OCC3(F)CCN(C(=O)OC(C)(C)C)CC3)no2)C(C)C)cc1]

To a solution of 2-[3-[(1-tert-butoxycarbonyl-4-fluoro-4-piperidyl)methoxy]isoxazol-5-yl]-3-methyl-butanoic acid (1.80 g, crude) in CH₂Cl₂ (20 mL) were added HATU (2.56 g, 6.74 mmol, 1.5 eq) and diisopropylethylamine (2.90 g, 22.5 mmol, 3.91 mL, 5 eq) followed by (2S,4R)-4-hydroxy-N-[(1S)-1-[4-(4-methylthiazol-5-yl)phenyl]ethyl]pyrrolidine-2-carboxamide (1.65 g, 4.50 mmol, 1 eq, HCl), and the reaction mixture was stirred at 25° C. for 1 hour. Water (10 mL) was then added, and the resulting mixture was extracted with EtOAc (3×20 mL). The combined organic extract was extracted with brine (3×20 mL), dried over anhydrous Na₂SO₄, filtered, and concentrated. The resulting crude product was purified by flash silica gel chromatography (gradient: 0-80% EtOAc in petroleum ether) to give the product mixture (1.6 g) as a white solid. Chiral separation by prep-SFC (40% EtOH in 0.1% NH₄OH) afforded tert-butyl 4-fluoro-4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate (950 mg, 1.27 umol, 28% yield) as a white solid (LC/MS (ESI) m/z: 714.1 [M+H]⁺) and tert-butyl 4-fluoro-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate (566 mg, 0.789 mmol, 17% yield) as white solid (LC/MS (ESI) m/z: 714.1 [M+H]⁺).

### Step 4: Preparation of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[2-[6-Chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from 4-fluoro-4-[[5-[(1R)-1-[(2S ,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

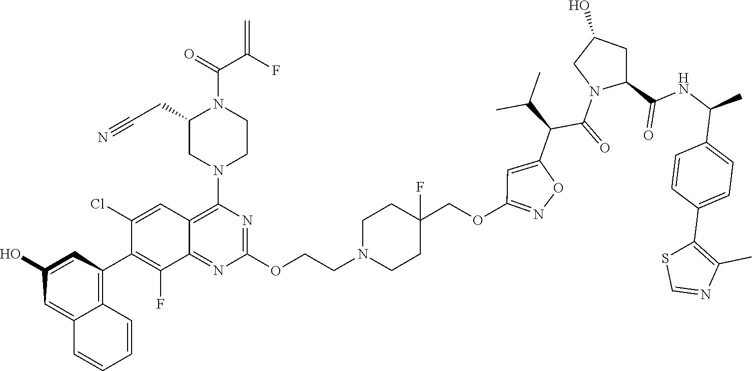

(formic acid salt, white solid). LC/MS (ESI) m/z: 1175.2 [M+H]⁺. ¹H-NMR (400 MHz, CD₃OD) δ 8.90-8.84 (m, 1H), 8.64 (d, J=7.2 Hz, 1H), 8.08 (s, 1H), 7.75 (d, J=8.4 Hz, 1H), 7.46-7.38 (m, 5H), 7.28-7.17 (m, 3H), 7.03 (d, J=2.6 Hz, 1H), 6.11-5.95 (m, 1H), 5.51-5.25 (m, 2H), 5.10-4.94 (m, 2H), 4.72-4.36 (m, 6H), 4.26 (d, J=19.2 Hz, 3H), 3.89-3.45 (m, 7H), 3.25-2.84 (m, 6H), 2.52-2.45 (m, 3H), 2.39-1.84 (m, 8H), 1.58-1.46 (m, 3H), 1.05 (d, J=6.4 Hz, 3H), 0.91-0.82 (m, 3H).

### Exemplary Synthesis of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

### Step 1: Preparation of tert-butyl 4-fluoro-4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate and tert-butyl 4-fluoro-4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate

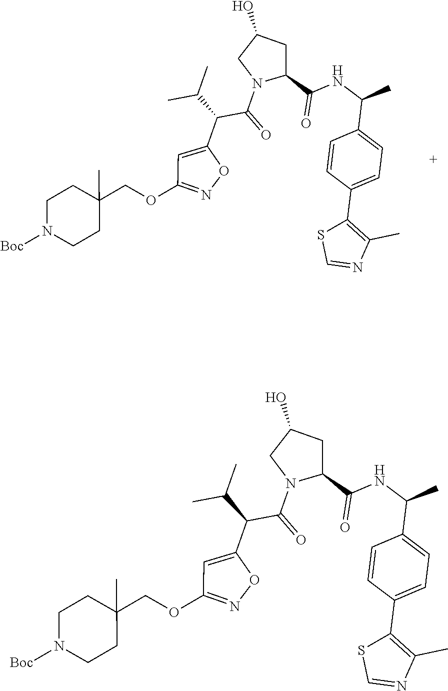 [SMILES: Cc1ncsc1-c1ccc([C@H](C)NC(=O)[C@@H]2C[C@@H](O)CN2C(=O)[C@H](c2cc(OCC3(C)CCN(C(=O)OC(C)(C)C)CC3)no2)C(C)C)cc1 ; Cc1ncsc1-c1ccc([C@H](C)NC(=O)[C@@H]2C[C@@H](O)CN2C(=O)[C@@H](c2cc(OCC3(C)CCN(C(=O)OC(C)(C)C)CC3)no2)C(C)C)cc1]

To 2-[3-[(1-tert-butoxycarbonyl-4-fluoro-4-piperidyl)methoxy]isoxazol-5-yl]-3-methyl-butanoic acid (2.4 g, 5.99 mmol, 1.0 eq) in DMF (24 mL) were added diisopropylethylamine (5.22 mL, 29.97 mmol, 5.0 eq) and HATU (2.73 g, 7.19 mmol, 1.2 eq,) and the reaction mixture was stirred at 25° C. for 0.5 hour. (2S,4R)-4-Hydroxy-N-[(1S)-1-[4-(2-methylpyrazol-3-yl)phenyl]ethyl]pyrrolidine-2-carboxamide (2.10 g, 5.99 mmol, 1.0 eq, HCl) was then added, and the resulting suspension was stirred at 25° C. for 1 hour. The reaction mixture was diluted with EtOAc (150 mL) and washed with water (3×50 mL) followed by brine (4×50 mL), then dried over Na₂SO₄, filtered, and concentrated. The resulting residue was purified by flash silica gel chromatography (gradient: 0-71% THF/Petroleum ether) to afford tert-butyl 4-fluoro-4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (2.72 g, 3.49 mmol, 58.27% yield) as a brown oil. LC/MS (ESI) m/z: 697.4 [M+H]⁺. Chiral separation by SFC (45% EtOH in 0.1% NH₄OH) afforded tert-butyl 4-fluoro-4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (735 mg, 1.04 mmol, 26.60% yield) as a white solid (LC/MS (ESI) m/z: 697.3 [M+H]⁺) and tert-butyl 4-fluoro-4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (796 mg, 1.13 mmol, 29% yield) as a white solid (LC/MS (ESI) m/z: 697.5 [M+H]⁺).

### Step 2: Preparation of (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

(2S,4R)-1-[(2R)-2-[3-[[1-[2-[6-Chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-(2-fluoroprop-2-enoyl)piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide starting from tert-butyl 4-fluoro-4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate.

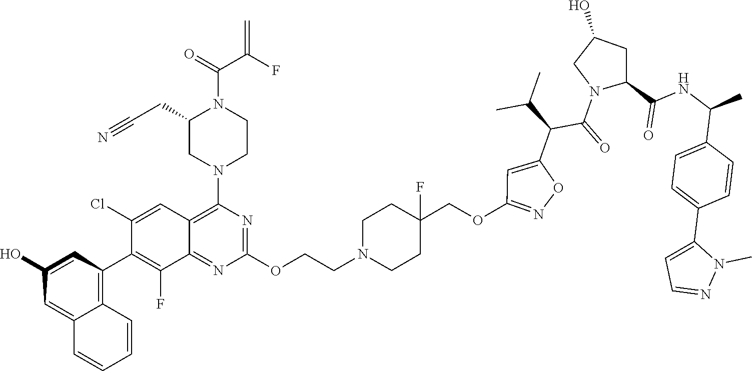

(formic acid salt, white solid). LC/MS (ESI) m/z: 1158.5 [M+H]⁺. ¹H-NMR (400 MHz, DMSO-d₆) δ 10.06 (s, 1H), 8.43 (d, J=7.6 Hz, 1H), 8.11 (s, 1H), 7.81 (d, J=8.4 Hz, 1H), 7.48-7.42 (m, 4H), 7.40-7.36 (m, 2H), 7.29 (d, J=2.0 Hz, 1H), 7.23-7.19 (m, 2H), 7.06 (d, J=2.4 Hz, 1H), 6.37 (d, J=2.0 Hz, 1H), 6.14 (s, 1H), 5.42 (dd, J=4.0, 18.0 Hz, 1H), 5.32 (d, J=19.2 Hz, 1H), 5.11 (d, J=3.2 Hz, 1H), 5.00-4.82 (m, 2H), 4.54-4.45 (m, 2H), 4.40-4.33 (m, 2H), 4.33-4.15 (m, 5H), 3.84 (s, 3H), 3.73-3.53 (m, 6H), 3.06 (dd, J=6.0, 17.2 Hz, 2H), 2.95-2.78 (m, 4H), 2.46-2.31 (m, 2H), 2.30-2.15 (m, 1H), 2.07-1.73 (m, 6H), 1.38 (d, J=6.8 Hz, 3H), 0.98-0.93 (m, 3H), 0.83-0.77 (m, 3H).

### Exemplary Synthesis of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[2-[6-chloro-4-[(3S)-3-(cyanomethyl)-4-prop-2-enoyl-piperazin-1- yl]-8-fluoro-7-(3-hydroxy-1-naphthyl)quinazolin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-fluoro-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate and 2-[(2S)-4-[6-chloro-8-fluoro-7-(3-hydroxy-1-naphthyl)-2-(2-oxoethoxy)quinazolin-4-yl]piperazin-2-yl]acetonitrile.

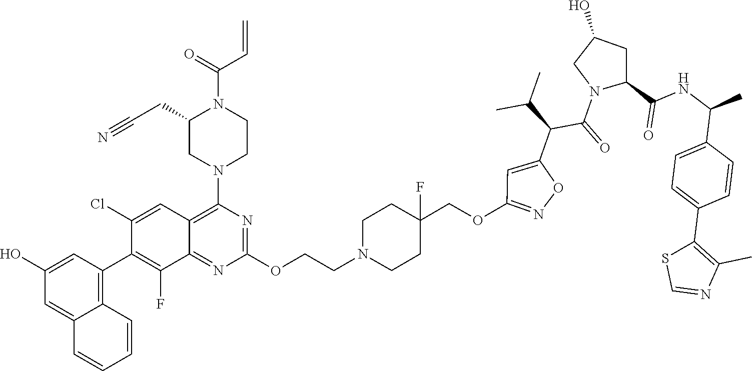 [SMILES: C=CC(=O)N1CCN(c2nc(OCCN3CCC(F)(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(formic acid salt, white solid). LC/MS (ESI) m/z: 1157.3 [M+H]⁺. ¹H-NMR (400 MHz, CD₃OD) δ 8.88 (s, 1H), 8.46 (s, 1H), 8.07 (s, 1H), 7.76 (d, J=8.0 Hz, 1H), 7.49-7.34 (m, 5H), 7.29-7.17 (m, 3H), 7.04 (d, J=2.4 Hz, 1H), 6.93-6.73 (m, 1H), 6.31 (d, J=16.4 Hz, 1H), 6.06-5.95 (m, 1H), 5.89-5.81 (m, 1H), 5.16-4.95 (m, 2H), 4.74-4.56 (m, 4H), 4.56-4.38 (m, 4H), 4.29-4.17 (m, 2H), 3.85-3.82 (m, 2H), 3.78-3.57 (m, 3H), 3.56-3.44 (m, 1H), 3.03 (s, 6H), 2.76-2.56 (m, 2H), 2.48 (s, 3H), 2.39-1.88 (m, 6H), 1.52 (d, J=7.2 Hz, 3H), 1.05 (d, J=6.8 Hz, 3H), 0.88 (d, J=6.8 Hz, 3H).

### Exemplary Synthesis of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[2-[6-Chloro-4-[(3S)-3-(cyanomethyl)-4-prop-2-enoyl-piperazin-1- yl]-8-fluoro-7-(3-hydroxy-1-naphthyl)quinazolin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate and 2-[(2S)-4-[6-chloro-8-fluoro-7-(3-hydroxy-1-naphthyl)-2-(2-oxoethoxy)quinazolin-4-yl]piperazin-2-yl]acetonitrile.

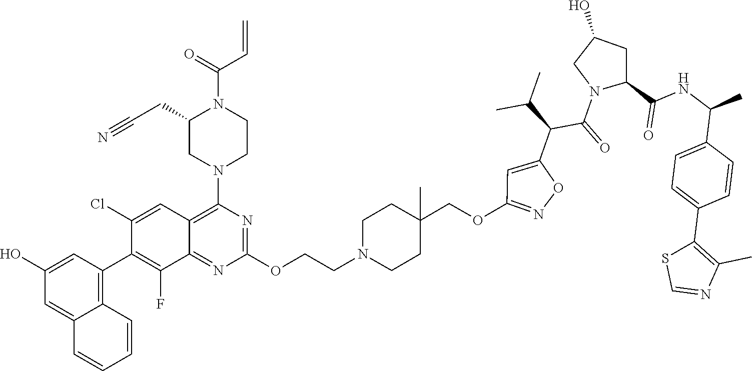 [SMILES: C=CC(=O)N1CCN(c2nc(OCCN3CCC(C)(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(formic acid salt, white solid). LC/MS (ESI) m/z: 1153.7 [M+H]⁺. ¹H-NMR (400 MHz, CD₃OD) δ 8.91-8.83 (m, 1H), 8.55-8.49 (m, 1H), 8.13-8.03 (m, 1H), 7.80-7.71 (m, 1H), 7.47-7.35 (m, 5H), 7.27 (d, J=2.0 Hz, 1H), 7.25-7.15 (m, 2H), 7.06-7.01 (m, 1H), 6.96-6.76 (m, 1H), 6.36-6.26 (m, 1H), 6.03-5.92 (m, 1H), 5.90-5.81 (m, 1H), 5.18-4.94 (m, 2H), 4.76-4.70 (m, 2H), 4.62-4.57 (m, 1H), 4.54-4.38 (m, 4H), 4.22-3.92 (m, 3H), 3.90-3.79 (m, 2H), 3.77-3.43 (m, 4H), 3.23-3.13 (m, 2H), 3.08-2.98 (m, 3H), 2.96-2.80 (m, 2H), 2.49-2.45 (m, 3H), 2.40-2.31 (m, 1H), 2.22-2.13 (m, 1H), 2.04-1.90 (m, 1H), 1.86-1.75 (m, 2H), 1.61-1.49 (m, 5H), 1.14-1.08 (m, 3H), 1.05 (d, J=6.4 Hz, 3H), 0.92-0.84 (m, 3H).

### Exemplary Synthesis of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methyl-methyl-amino]isoxazol-5-yl]-3-methyl-butanoyl]-4-hydroxy-N-[(1S)-1-[4-(4-methylthiazol-5-yl)phenyl]ethyl]pyrrolidine-2-carboxamide

### Step 1: Preparation of methyl 3-methyl-2-[3-(1,1,2,2,3,3,4,4,4-nonafluorobutylsulfonyloxy)isoxazol-5-yl]butanoate

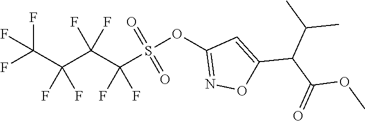 [SMILES: COC(=O)C(c1cc(OS(=O)(=O)C(F)(F)C(F)(F)C(F)(F)C(F)(F)F)no1)C(C)C]

To a mixture of methyl 2-(3-hydroxyisoxazol-5-yl)-3-methyl-butanoate (2.00 g, 10.0 mmol, 1 eq) and K₂CO₃ (763 mg, 5.52 mmol, 0.55 eq) in CH₃CN (20 mL) at 0° C. was added 1,1,2,2,3,3,4,4,4-nonafluorobutane-1-sulfonyl fluoride (6.07 g, 20.1 mmol, 3.53 mL, 2 eq) dropwise, and the reaction mixture was stirred under N₂ at 25° C. for 16 hours. The mixture was poured onto saturated aqueous NH₄Cl (30 mL), and the resulting mixture was extracted with EtOAc (3×30 mL). The combined organic extract was washed with brine (30 mL), dried over anhydrous Na₂SO₄, filtered, and concentrated. The resulting crude product was purified by flash silica gel chromatography (gradient: 0-2% THF in petroleum ether) to give methyl 3-methyl-2-[3-(1,1,2,2,3,3,4,4,4-nonafluorobutylsulfonyloxy)isoxazol-5-yl]butanoate (3.58 g, 7.44 mmol, 74% yield) as a colorless oil. LC/MS (ESI) m/z: 482.0 [M+H]⁺.

### Step 2: Preparation of tert-butyl 4-[[[5-(1-methoxycarbonyl-2-methyl-propyl)isoxazol-3-yl]-methyl-amino]methyl]piperidine-1-carboxylate

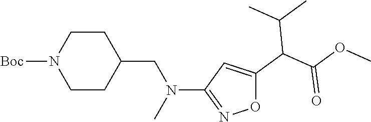 [SMILES: COC(=O)C(c1cc(N(C)CC2CCN(C(=O)OC(C)(C)C)CC2)no1)C(C)C]

To a solution of tert-butyl 4-(methylaminomethyl)piperidine-1-carboxylate (500 mg, 2.19 mmol, 1 eq) in DMA (10 mL) were added triethylamine (288 mg, 2.85 mmol, 0.396 mL, 1.3 eq) and methyl 3-methyl-2-[3-(1,1,2,2,3,3,4,4,4-nonafluorobutylsulfonyloxy)isoxazol-5-yl]butanoate (1.05 g, 2.19 mmol, 1 eq), and the reaction mixture was stirred under N₂ at 140° C. for 4 hours. Additional methyl 3-methyl-2-[3-(1,1,2,2,3,3,4,4,4-nonafluorobutylsulfonyloxy)isoxazol-5-yl]butanoate (1.05 g, 2.19 mmol, 1 eq) and triethylamine (288 mg, 2.85 mmol, 0.396 mL, 1.3 eq) were then added, and the reaction mixture was stirred under N₂ at 140° C. for 4 hours. The reaction was cooled to room to room temperature, and then poured onto water (30 mL). The resulting mixture was extracted with EtOAc (3×30 mL), and the combined organic extracts was washed with brine (3×30 mL), dried over anhydrous Na₂SO₄, filtered, and concentrated. The resulting crude product was purified by flash silica gel chromatography (gradient: 0-10% THF in petroleum ether) to give tert-butyl 4-[[[5-(1-methoxycarbonyl-2-methyl-propyl)isoxazol-3-yl]-methyl-amino]methyl]piperidine-1-carboxylate (635 mg, 1.55 mmol, 71% yield) as a yellow oil. LC/MS (ESI) m/z: 410.3 [M+H]⁺.

### Step 3: Preparation of 2-[3-[(1-tert-butoxycarbonyl-4-piperidyl)methyl-methyl-amino]isoxazol-5-yl]-3-methyl-butanoic Acid

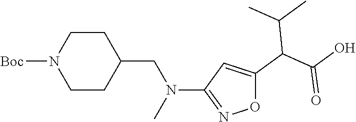 [SMILES: CC(C)C(C(=O)O)c1cc(N(C)CC2CCN(C(=O)OC(C)(C)C)CC2)no1]

To a solution of tert-butyl 4-[[[5-(1-methoxycarbonyl-2-methyl-propyl)isoxazol-3-yl]-methyl-amino]methyl]piperidine-1-carboxylate (780 mg, 1.90 mmol, 1 eq) in THF (4 mL) was added a solution of LiOH.H₂O (160 mg, 3.81 mmol, 2 eq) in water (4 mL), and the reaction mixture was stirred under N₂ at 50° C. for 4 hours. The mixture was concentrated under reduced pressure to remove THF, and the resulting mixture was diluted with water (10 mL). The pH was adjusted to ˜5 by addition of 2N aqueous HCl, and the resulting mixture was lyophilized to give 2-[3-[(1-tert-butoxycarbonyl-4-piperidyl)methyl-methyl-amino]isoxazol-5-yl]-3-methyl-butanoic acid (750 mg, crude) as a yellow oil. LC/MS (ESI) m/z: 396.3 [M+H]⁺.

### Step 4: Preparation of tert-butyl 4-[[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-methyl-amino]methyl]piperidine-1-carboxylate & tert-butyl 4-[[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-methyl-amino]methyl]piperidine-1-carboxylate

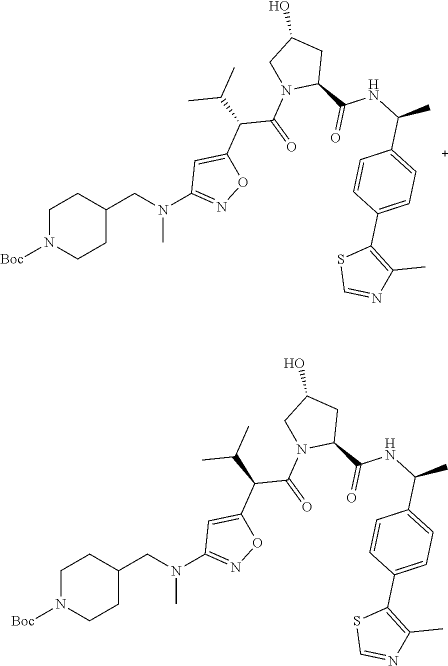 [SMILES: Cc1ncsc1-c1ccc([C@H](C)NC(=O)[C@@H]2C[C@@H](O)CN2C(=O)[C@H](c2cc(N(C)CC3CCN(C(=O)OC(C)(C)C)CC3)no2)C(C)C)cc1 ; Cc1ncsc1-c1ccc([C@H](C)NC(=O)[C@@H]2C[C@@H](O)CN2C(=O)[C@@H](c2cc(N(C)CC3CCN(C(=O)OC(C)(C)C)CC3)no2)C(C)C)cc1]

To a solution of 2-[3-[(1-tert-butoxycarbonyl-4-piperidyl)methyl-methyl-amino]isoxazol-5-yl]-3-methyl-butanoic acid (750 mg, 1.90 mmol, 1 eq) in CH₂Cl₂ (15 mL) were added diisopropylethylamine (1.23 g, 9.48 mmol, 1.65 mL, 5 eq), HATU (865 mg, 2.28 mmol, 1.2 eq), and (2S,4R)-4-hydroxy-N-[(1S)-1-[4-(4-methylthiazol-5-yl)phenyl]ethyl]pyrrolidine-2-carboxamide (698 mg, 1.90 mmol, 1 eq, HCl), and the reaction mixture was stirred under N₂ at 25° C. for 1 hour. The reaction mixture was poured onto water (40 mL) and extracted with CH₂Cl₂ (2×30 mL). The combined organic extract was washed with brine (30 mL), dried over anhydrous Na₂SO₄, filtered, and concentrated. The resulting crude product was purified by flash silica gel chromatography (gradient: 0-100% EtOAc in petroleum ether) to give tert-butyl 4-[[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-methyl-amino]methyl]piperidine-1-carboxylate (1.08 g, 1.52 mmol, 80% yield) as a yellow solid. LC/MS (ESI) m/z: 709.4 [M+H]⁺. This material (990 mg, 1.40 mmol, 1 eq) was separated by SFC (35% isopropanol in 0.1% NH₄OH) to give tert-butyl 4-[[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-methyl-amino]methyl]piperidine-1-carboxylate (520 mg, 0.734 mmol, 53% yield) as a white solid (LC/MS (ESI) m/z: 709.5 [M+H]⁺) and tert-butyl 4-[[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-methyl-amino]methyl]piperidine-1-carboxylate (281 mg, 0.396 mmol, 28% yield) as a white solid (LC/MS (ESI) m/z: 709.4 [M+H]⁺).

### Step 5: Preparation of (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methyl-methyl-amino]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[2-[6-Chloro-4-[(3S)-3-(cyanomethyl)-4-prop-2-enoyl-piperazin-1- yl]-8-fluoro-7-(3-hydroxy-1-naphthyl)quinazolin-2-yl]oxyethyl]-4-piperidyl]methyl-methyl-amino]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-[[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-methyl-amino]methyl]piperidine-1-carboxylate and 2-[(2S)-4-[6-chloro-8-fluoro-7-(3-hydroxy-1-naphthyl)-2-(2-oxoethoxy)quinazolin-4-yl]piperazin-2-yl]acetonitrile.

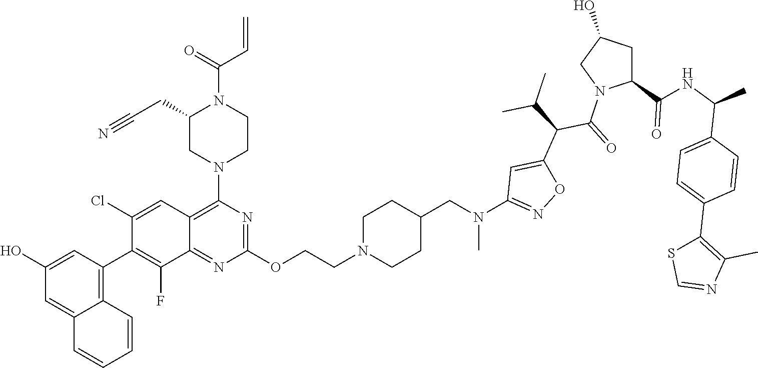 [SMILES: C=CC(=O)N1CCN(c2nc(OCCN3CCC(CN(C)c4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(formic acid salt, white solid). LC/MS (ESI) m/z: 1152.7 [M+H]⁺. ¹H-NMR (400 MHz, CD₃OD) δ 8.93-8.82 (m, 1H), 8.52 (s, 1H), 8.09 (s, 1H), 7.75 (d, J=8.4 Hz, 1H), 7.50-7.33 (m, 5H), 7.30-7.13 (m, 3H), 7.04 (d, J=2.4 Hz, 1H), 6.97-6.74 (m, 1H), 6.31 (d, J=16.8 Hz, 1H), 6.05-5.93 (m, 1H), 5.85 (d, J=9.2 Hz, 1H), 5.05-5.01 (m, 1H), 4.75-4.69 (m, 2H), 4.55-4.10 (m, 5H), 4.02-3.66 (m, 4H), 3.59 (d, J=10.0 Hz, 2H), 3.44-3.34 (m, 2H), 3.25-2.98 (m, 6H), 2.97-2.87 (m, 3H), 2.69-2.50 (m, 2H), 2.49-2.43 (m, 3H), 2.41-2.30 (m, 1H), 2.22-2.11 (m, 1H), 2.06-1.72 (m, 4H), 1.64-1.24 (m, 6H), 1.11-0.98 (m, 3H), 0.95-0.80 (m, 3H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-methyl-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-[(2-ethoxy-2-oxo-ethoxy)methyl]-4-methyl-piperidine-1-carboxylate

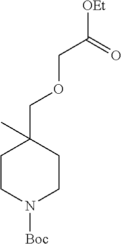 [SMILES: CCOC(=O)COCC1(C)CCN(C(=O)OC(C)(C)C)CC1]

To a mixture of tert-butyl 4-(hydroxymethyl)-4-methyl-piperidine-1-carboxylate (1.00 g, 4.36 mmol, 1 eq) and Rh₂(OAc)₄ (96 mg, 0.22 mmol, 0.05 eq) in CH₂Cl₂ (15 mL) at 0° C. was added a solution of ethyl 2-diazoacetate (597 mg, 5.23 mmol, 1.2 eq) in CH₂Cl₂ (5 mL) slowly, and the resulting suspension was stirred at 25° C. for 12 hours. The reaction mixture was diluted with water (20 mL) and extracted with CH₂Cl₂ (3×20 mL). The organic extracts were combined and washed with brine (20 mL), dried over Na₂SO₄, filtered, and concentrated. The resulting residue was purified by flash chromatography on SiO₂ (gradient: 0-10% EtOAc in petroleum ether) to give tert-butyl 4-[(2-ethoxy-2-oxo-ethoxy)methyl]-4-methyl-piperidine-1-carboxylate (530 mg, 1.68 mmol, 39% yield) as a colorless oil. ¹H-NMR (400 MHz, CDCl₃) δ 4.24-4.18 (m, 2H), 4.06 (s, 2H), 3.66-3.57 (m, 2H), 3.26 (s, 2H), 3.25-3.15 (m, 2H), 1.58-1.49 (m, 2H), 1.46 (s, 9H), 1.33-1.27 (m, 5H), 1.03 (s, 3H).

### Step 2: Preparation of 2-[(1-tert-butoxycarbonyl-4-methyl-4-piperidyl)methoxy]acetic Acid

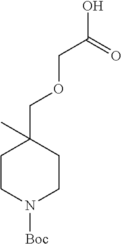 [SMILES: CC1(COCC(=O)O)CCN(C(=O)OC(C)(C)C)CC1]

To tert-butyl 4-[(2-ethoxy-2-oxo-ethoxy)methyl]-4-methyl-piperidine-1-carboxylate (530 mg, 1.68 mmol, 1 eq) in THF (5 mL) and H₂O (5 mL) was added LiOH.H₂O (353 mg, 8.40 mmol, 5 eq), and the reaction mixture was stirred at 25° C. for 2 hours. The pH of the reaction mixture was adjusted to ˜4-5 by addition of 2N aqueous HCl, and the resulting acidic mixture was lyophilized to give crude 2-[(1-tert-butoxycarbonyl-4-methyl-4-piperidyl)methoxy]acetic acid (470 mg) as a colorless oil. ¹H-NMR (400 MHz, CD₃OD) δ 4.06 (s, 2H), 3.64-3.52 (m, 2H), 3.33-3.32 (m, 2H), 3.30-3.18 (m, 2H), 1.60-1.51 (m, 2H), 1.47 (s, 9H), 1.34-1.27 (m, 2H), 1.03 (s, 3H).

### Step 3: Preparation of tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-4-methyl-piperidine-1-carboxylate

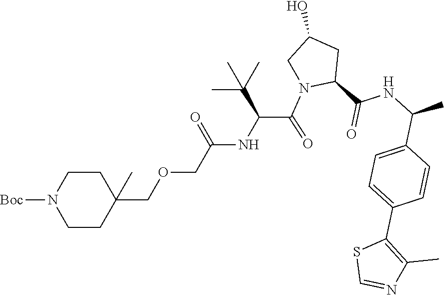 [SMILES: Cc1ncsc1-c1ccc([C@H](C)NC(=O)[C@@H]2C[C@@H](O)CN2C(=O)[C@@H](NC(=O)COCC2(C)CCN(C(=O)OC(C)(C)C)CC2)C(C)(C)C)cc1]

To a solution of 2-[(1-tert-butoxycarbonyl-4-methyl-4-piperidyl)methoxy]acetic acid (470 mg, 1.64 mmol, 1 eq) in CH₂Cl₂ (8 mL) were added diisopropylethylamine (21 mg, 1.64 mmol, 0.285 mL, 1 eq) and HATU (622 mg, 1.64 mmol, 1 eq), and the reaction mixture was stirred at 20° C. for 0.5 hour. (2S,4R)-1-[(2S)-2-Amino-3,3-dimethyl-butanoyl]-4-hydroxy-N-[(1S)-1-[4-(4-methylthiazol-5-yl)phenyl]ethyl]pyrrolidine-2-carboxamide (727 mg, 1.64 mmol, 1 eq) was then added, and the reaction mixture was stirred at 20° C. for 2 hours. The mixture was washed with brine (15 mL), dried with anhydrous Na₂SO₄, filtered, and concentrated. The resulting residue was purified by flash chromatography on SiO₂ (gradient: 0-100% EtOAc in petroleum ether) to afford tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-4-methyl-piperidine-1-carboxylate (1.09 g, 1.53 mmol, 93% yield) as a light yellow solid. LC/MS (ESI) m/z: 714.2 [M+H]⁺.

### Step 4: Preparation of (2S,4R)-1-[(2S)-3,3-dimethyl-2-[[2-[(4-methyl-4-piperidyl)methoxy]acetyl]amino]butanoyl]-4-hydroxy-N-[(1S)-1-[4-(4-methylthiazol-5-yl)phenyl]ethyl]pyrrolidine-2-carboxamide

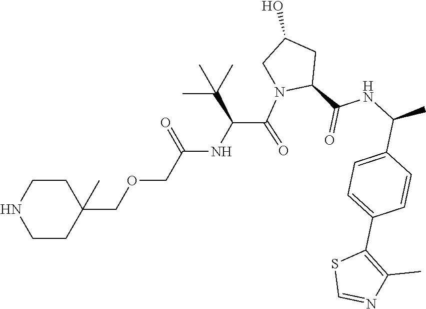 [SMILES: Cc1ncsc1-c1ccc([C@H](C)NC(=O)[C@@H]2C[C@@H](O)CN2C(=O)[C@@H](NC(=O)COCC2(C)CCNCC2)C(C)(C)C)cc1]

To a solution of tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-4-methyl-piperidine-1-carboxylate (1.09 g, 1.53 mmol, 1.0 eq) in CH₂Cl₂ (5 mL) was added HCl (4N in CH₃OH, 5 mL), and the reaction mixture was stirred at 25° C. for 1 hour. The mixture was concentrated in vacuum, and the resulting residue was diluted with water (15 mL). The pH of the aqueous mixture was adjusted to ˜7-8 by addition of saturated aqueous NaHCO₃, and resulting basic aqueous mixture was extracted with EtOAc (3×20 mL). The combined organic extract was washed with brine (20 mL), dried over Na₂SO₄, filtered, and concentrated to give (2S,4R)-1-[(2S)-3,3-dimethyl-2-[[2-[(4-methyl-4-piperidyl)methoxy]acetyl]amino]butanoyl]-4-hydroxy-N-[(1S)-1-[4-(4-methylthiazol-5-yl)phenyl]ethyl]pyrrolidine-2-carboxamide (730 mg, 1.09 mmol, 72% yield) as a yellow oil. 614.5 [M+H]⁺.

### Step 5: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-methyl-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-methyl-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl- piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from (2S,4R)-1-[(2S)-3,3-dimethyl-2-[[2-[(4-methyl-4-piperidyl)methoxy]acetyl]amino]butanoyl]-4-hydroxy-N-[(1S)-1-[4-(4-methylthiazol-5-yl)phenyl]ethyl]pyrrolidine-2-carboxamide and 2-[(2S)-4-[6-chloro-8-fluoro-7-(3-hydroxy-1-naphthyl)-2-(2-oxoethoxy)quinazolin-4-yl]piperazin-2-yl]acetonitrile.

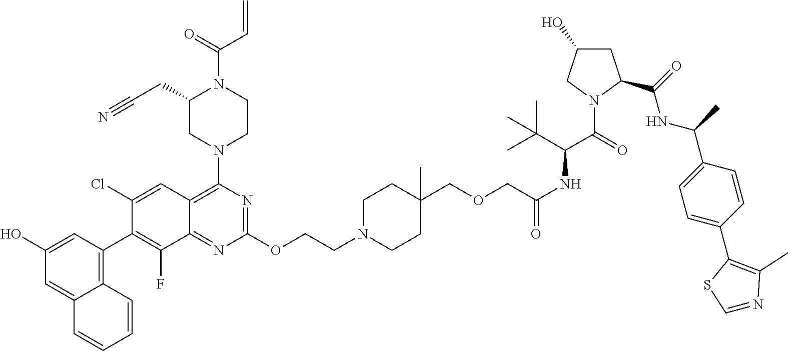 [SMILES: C=CC(=O)N1CCN(c2nc(OCCN3CCC(C)(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(formic acid salt, white solid). LC/MS (ESI) m/z: 1157.7 [M+H]⁺. ¹H-NMR (400 MHz, CD₃OD) δ 8.88-8.82 (m, 1H), 8.57-8.51 (m, 1H), 8.09-8.02 (m, 1H), 7.78-7.71 (m, 1H), 7.46-7.33 (m, 5H), 7.30-7.14 (m, 3H), 7.04 (d, J=2.0 Hz, 1H), 6.95-6.72 (m, 1H), 6.38-6.26 (m, 1H), 5.90-5.79 (m, 1H), 5.14-4.95 (m, 3H), 4.76-4.72 (m, 2H), 4.60-4.55 (m, 2H), 4.53-4.47 (m, 2H), 4.45-4.41 (m, 1H), 4.07 (s, 1H), 4.05-3.92 (m, 2H), 3.91-3.81 (m, 2H), 3.79-3.67 (m, 2H), 3.62-3.48 (m, 1H), 3.44-3.33 (m, 2H), 3.28-3.24 (m, 1H), 3.19-3.08 (m, 2H), 3.07-2.88 (m, 4H), 2.49-2.42 (m, 3H), 2.26-2.16 (m 1H), 1.99-1.89 (m, 1H), 1.87-1.73 (m, 2H), 1.62-1.53 (m, 2H), 1.46 (d, J=7.0 Hz, 3H), 1.09 (s, 3H), 1.06-0.98 (m, 9H).

### Exemplary Synthesis of (2S,4R)-1-[(2S)-2-[[2-[[1-[2-[6-chloro-8-fluoro-7-(3-hydroxy-1-naphthyl)-4-[(2S)-2-methyl-4-prop-2-enoyl-piperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of benzyl 4-[(2-tert-butoxy-2-oxo-ethoxy)methyl]piperidine-1-carboxylate

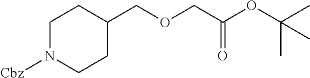 [SMILES: CC(C)(C)OC(=O)COCC1CCN(C(=O)OCc2ccccc2)CC1]

To benzyl 4-(hydroxymethyl)piperidine-1-carboxylate (10 g, 40.11 mmol, 1 eq) in DMF (150 mL) was added NaH (3.21 g, 80.22 mmol, 60% purity, 2 eq) in one portion at 0° C. under N₂, and the resulting mixture was stirred at 0° C. for 15 minutes. tert-Butyl 2-bromoacetate (15.65 g, 80.22 mmol, 11.85 mL, 2 eq) was then added, and the resulting mixture was stirred at 20° C. for 16 hours. The reaction mixture was quenched with water (150 mL) and stirred for 15 minutes. The aqueous phase was extracted with EtOAc (3×200 mL), and the combined organic extract was washed with brine (3×150 mL), dried over anhydrous Na₂SO₄, filtered, and concentrated. The resulting residue was purified by flash chromatography on SiO₂ (gradient: 0-13% EtOAc in petroleum ether) to afford benzyl 4-[(2-tert-butoxy-2-oxo-ethoxy)methyl]piperidine-1-carboxylate (3.9 g, 10.73 mmol, 27% yield) as a colorless oil. ¹H-NMR (400 MHz, CDCl₃) δ 7.37-7.32 (m, 5H), 5.13 (s, 2H), 4.21 (s, 2H), 3.94 (s, 2H), 3.37 (d, J=4.0 Hz, 2H), 2.79 (s, 2H), 1.84-1.74 (m, 3H), 1.48 (s, 9H), 1.25-1.17 (m, 2H).

### Step 2: Preparation of tert-butyl 2-(4-piperidylmethoxy)acetate

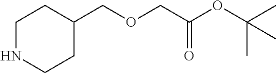 [SMILES: CC(C)(C)OC(=O)COCC1CCNCC1]

To a solution of benzyl 4-[(2-tert-butoxy-2-oxo-ethoxy)methyl]piperidine-1-carboxylate (3.9 g, 10.73 mmol, 1 eq) and NH₄OH (283.92 mg, 2.27 mmol, 28% purity) in CH₃OH (100 mL) was added Pd/C (400 mg, 10% purity) under N₂, and the resulting suspension was degassed under vacuum and purged with H₂ several times. The reaction mixture was then stirred under H₂ (15 psi) at 20° C. for 16 hours. The reaction mixture was filtered, and the filtrate was concentrated to afford tert-butyl 2-(4-piperidylmethoxy)acetate (2.4 g, 10.47 mmol, 98% yield) as a yellow oil. ¹H-NMR (400 MHz, DMSO-d₆) δ 3.93 (s, 2H), 3.25 (d, J=4.0 Hz, 2H), 2.94 (d, J=12.0 Hz, 2H), 2.44 (s, 2H), 1.59 (d, J=12.0 Hz, 3H), 1.42 (s, 9H), 1.11-1.02 (m, 2H).

### Step 3: Preparation of tert-butyl 2-[[1-(2-hydroxyethyl)-4-piperidyl]methoxy]acetate

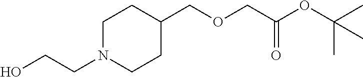 [SMILES: CC(C)(C)OC(=O)COCC1CCN(CCO)CC1]

To tert-butyl 2-(4-piperidylmethoxy)acetate (1.8 g, 7.85 mmol, 1 eq) in CH₃CN (20 mL) was added 2-iodoethanol (2 g, 11.77 mmol, 1.5 eq) followed by K₂CO₃ (2 g, 15.70 mmol, 2 eq), and the reaction mixture was stirred at 20° C. for 12 hours. The reaction mixture was filtered, and the filtrate was concentrated. The resulting residue was purified by flash chromatography on SiO₂ (gradient: 0-40% THF in CH₂Cl₂) to afford tert-butyl 2-[[1-(2-hydroxyethyl)-4-piperidyl]methoxy]acetate (1.14 g, 4.17 mmol, 53% yield) as a yellow oil. LC/MS (ESI) m/z: 274.2 [M+H]⁺. ¹H-NMR (400 MHz, CDCl₃) δ 3.95 (s, 2H), 3.68-3.63 (m, 2H), 3.38 (d, J=8.0 Hz, 2H), 3.06-2.97 (m, 2H), 2.63-2.57 (m, 2H), 2.21-2.11 (m, 2H), 1.88-1.78 (m, 2H), 1.77-1.65 (m, 1H), 1.48 (s, 9H), 1.45-1.42 (m, 1H), 1.42-1.31 (m, 2H).

### Step 4: Preparation of tert-butyl (3S)-4-(7-bromo-2, 6-dichloro-8-fluoro-quinazolin-4-yl)-3-methyl-piperazine-1-carboxylate

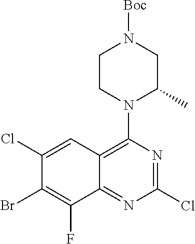 [SMILES: C[C@H]1CN(C(=O)OC(C)(C)C)CCN1c1nc(Cl)nc2c(F)c(Br)c(Cl)cc12]

To a mixture of 7-bromo-2,4,6-trichloro-8-fluoro-quinazoline (500 mg, 1.51 mmol, 1 eq) and tert-butyl (3S)-3-methylpiperazine-1-carboxylate (303 mg, 1.51 mmol, 1 eq) in CH₂Cl₂ (10 mL) was added diisopropylethylamine (586 mg, 4.54 mmol, 3 eq), and the reaction mixture was stirred at 20° C. under N₂ for 2 hours. The mixture was poured onto water (20 mL) and extracted with CH₂Cl₂ (3×20 mL). The combined organic extract was washed with brine, dried over Na₂SO₄, filtered, and concentrated. The resulting residue was purified by flash chromatography on SiO₂ (gradient: 0-10% EtOAc in petroleum ether) to afford tert-butyl (3S)-4-(7-bromo-2,6-dichloro-8-fluoro-quinazolin-4-yl)-3-methyl-piperazine-1-carboxylate (580 mg, 1.16 mmol, 77% yield) as a yellow solid. LC/MS (ESI) m/z: 494.9 [M+H]⁺.

### Step 5: Preparation of tert-butyl (3S)-4-[7-bromo-2-[2-[4-[(2-tert-butoxy-2-oxo-ethoxy)methyl]-1-piperidyl]ethoxy]-6-chloro-8-fluoro-quinazolin-4-yl]-3-methyl-piperazine-1-carboxylate

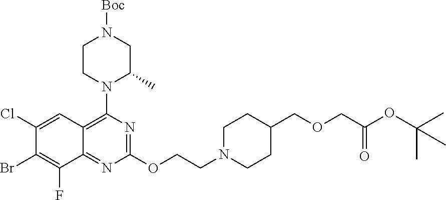 [SMILES: C[C@H]1CN(C(=O)OC(C)(C)C)CCN1c1nc(OCCN2CCC(COCC(=O)OC(C)(C)C)CC2)nc2c(F)c(Br)c(Cl)cc12]

To tert-butyl (3S)-4-(7-bromo-2,6-dichloro-8-fluoro-quinazolin-4-yl)-3-methyl-piperazine-1-carboxylate (580 mg, 1.17 mmol, 1 eq) in CH₃CN (3 mL) at 25° C. were added tert-butyl 2-[[1-(2-hydroxyethyl)-4-piperidyl]methoxy]acetate (417 mg, 1.53 mmol, 1.3 eq), Cs₂CO₃ (497 mg, 1.53 mmol, 1.3 eq), and DABCO (13 mg, 0.117 mmol, 0.1 eq) under N₂ atmosphere, and the reaction mixture was stirred at 50° C. for 2 hours. Water (10 mL) was then added, and the resulting mixture was extracted with EtOAc (3×20 mL). The combined organic extract was washed with brine (20 mL), dried over anhydrous Na₂SO₄, filtered, and concentrated. The resulting residue was purified by flash chromatography on SiO₂ (gradient: 0-50% EtOAc in petroleum ether) to afford tert-butyl (3S)-4-[7-bromo-2-[2-[4-[(2-tert-butoxy-2-oxo-ethoxy)methyl]-1-piperidyl]ethoxy]-6-chloro-8-fluoro-quinazolin-4-yl]-3-methyl-piperazine-1-carboxylate (656 mg, 780.64 umol, 66.5% yield) as a yellow solid. LC/MS (ESI) m/z: 732.3 [M+H]⁺.

### Step 6: Preparation of tert-butyl (3S)-4-[2-[2-[4-[(2-tert-butoxy-2-oxo-ethoxy)methyl]-1-piperidyl]ethoxy]-6-chloro-8-fluoro-7-(3-hydroxy-1-naphthyl)quinazolin-4-yl]-3-methyl-piperazine-1-carboxylate

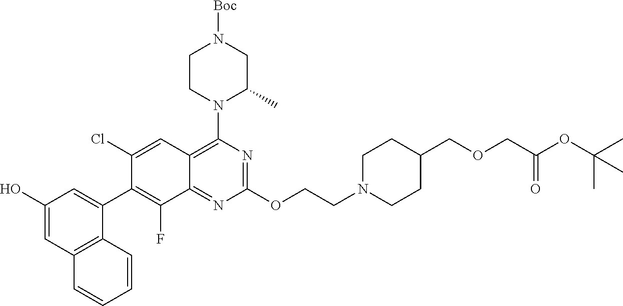 [SMILES: C[C@H]1CN(C(=O)OC(C)(C)C)CCN1c1nc(OCCN2CCC(COCC(=O)OC(C)(C)C)CC2)nc2c(F)c(-c3cc(O)cc4ccccc34)c(Cl)cc12]

To a solution of tert-butyl (3S)-4-[7-bromo-2-[2-[4-[(2-tert-butoxy-2-oxo-ethoxy)methyl]-1-piperidyl]ethoxy]-6-chloro-8-fluoro-quinazolin-4-yl]-3-methyl-piperazine-1-carboxylate (300 mg, 0.41 mmol, 1 eq) and 4-(4,4,5,5-tetramethyl-1,3,2-dioxaborolan-2-yl)naphthalen-2-ol (144 mg, 0.533 mmol, 1.3 eq) in THF (3 mL) were added XPhos-Pd-G3 (17 mg, 0.021 mmol, 0.05 eq) and K₃PO₄ (1.5 M, 821 uL, 3 eq), and the reaction mixture was stirred at 50° C. for 2 hours under N₂. Water (10 mL) was then added, and the resulting mixture was extracted with EtOAc (3×20 mL). The combined organic extracts were washed with brine (20 mL), dried over Na₂SO₄, filtered, and concentrated. The resulting residue was purified by flash chromatography on SiO₂ (gradient: 0-60% THF in petroleum ether) to afford tert-butyl (3S)-4-[2-[2-[4-[(2-tert-butoxy-2-oxo-ethoxy)methyl]-1-piperidyl]ethoxy]-6-chloro-8-fluoro-7-(3-hydroxy-1-naphthyl)quinazolin-4-yl]-3-methyl-piperazine-1-carboxylate (240 mg, 302.13 umol, 74% yield) as a yellow solid. LC/MS (ESI) m/z: 794.5 [M+H]⁺.

### Step 7: Preparation of 2-[[1-[2-[4-[(2S)-4-tert-butoxycarbonyl-2-methyl-piperazin-1-yl]-6-chloro-8-fluoro-7-(3-hydroxy-1-naphthyl)quinazolin-2-yl]oxyethyl]-4-piperidyl]methoxy]acetic Acid

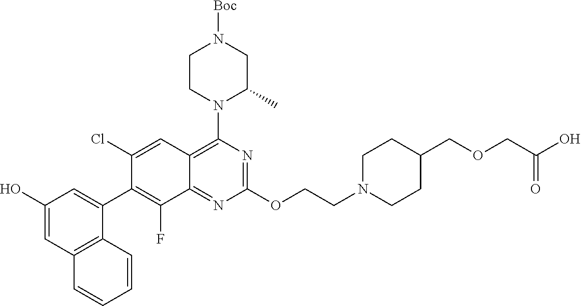 [SMILES: C[C@H]1CN(C(=O)OC(C)(C)C)CCN1c1nc(OCCN2CCC(COCC(=O)O)CC2)nc2c(F)c(-c3cc(O)cc4ccccc34)c(Cl)cc12]

To tert-butyl (3S)-4-[2-[2-[4-[(2-tert-butoxy-2-oxo-ethoxy)methyl]-1-piperidyl]ethoxy]-6-chloro-8-fluoro-7-(3-hydroxy-1-naphthyl)quinazolin-4-yl]-3-methyl-piperazine-1-carboxylate (240 mg, 0.302 mmol, 1 eq) in CH₃OH (1 mL), H₂O (1 mL), and THF (1 mL) at 20° C. was added LiOH.H₂O (380 mg, 9.06 mmol, 30 eq), and the reaction mixture was stirred under argon for 2 hours. The pH of the reaction mixture was adjusted to ˜3 by addition of aqueous HCl (2N), and the resulting acidic mixture was lyophilized to afford 2-[[1-[2-[4-[(2S)-4-tert-butoxycarbonyl-2-methyl-piperazin-1-yl]-6-chloro-8-fluoro-7-(3-hydroxy-1-naphthyl) quinazolin-2-yl]oxyethyl]-4-piperidyl]methoxy]acetic acid (223 mg, 302 mmol) as a white solid. LC/MS (ESI) m/z: 738.3 [M+H]⁺.

### Step 8: Preparation of tert-butyl (3S)-4-[6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)quinazolin-4-yl]-3-methyl-piperazine-1-carboxylate

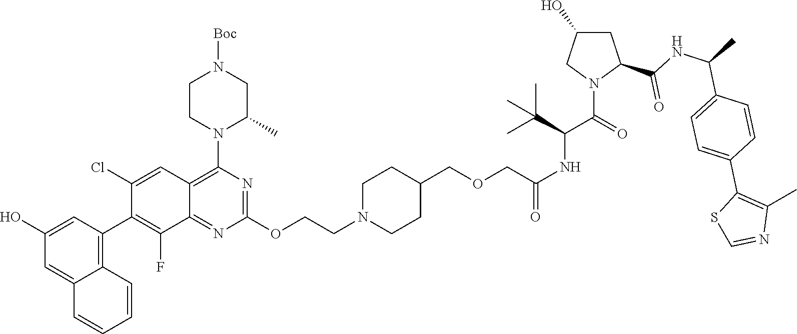 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCN(C(=O)OC(C)(C)C)C[C@@H]4C)c4cc(Cl)c(-c5cc(O)cc6ccccc56)c(F)c4n3)CC2)C(C)(C)C)cc1]

To a mixture of 2-[[1-[2-[4-[(2S)-4-tert-butoxycarbonyl-2-methyl-piperazin-1-yl]-6-chloro-8-fluoro-7-(3-hydroxy-1-naphthyl)quinazolin-2-yl]oxyethyl]-4-piperidyl]methoxy]acetic acid (223 mg, 0.302 mmol, 1 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (134 mg, 0.279 mmol, 9.24e-1 eq, HCl) in DMF (6 mL) at 20° C. were added diisopropylethylamine (195 mg, 1.51 mmol, 5 eq) and HATU (115 mg, 0.302 mmol, 1 eq), and the reaction mixture was stirred at 20° C. for 2 hours. Saturated aqueous K₂CO₃ (6 mL) was then added, and the resulting mixture was stirred for 0.5 hours. The mixture was diluted with water (10 mL) and extracted with EtOAc (3×30 mL). The combined organic extracts were washed with brine (30 mL) and concentrated. The resulting residue was purified by flash chromatography on SiO₂ (gradient: 0-1% CH₃OH in THF) to afford tert-butyl (3S)-4-[6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)quinazolin-4-yl]-3-methyl-piperazine-1-carboxylate (289 mg) as a yellow solid. LC/MS (ESI) m/z: 583.2 [M/2+H]⁺.

### Step 9: Preparation of (2S,4R)-1-[(2S)-2-[[2-[[1-[2-[6-chloro-8-fluoro-7-(3-hydroxy-1-naphthyl)-4-[(2S)-2-methylpiperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

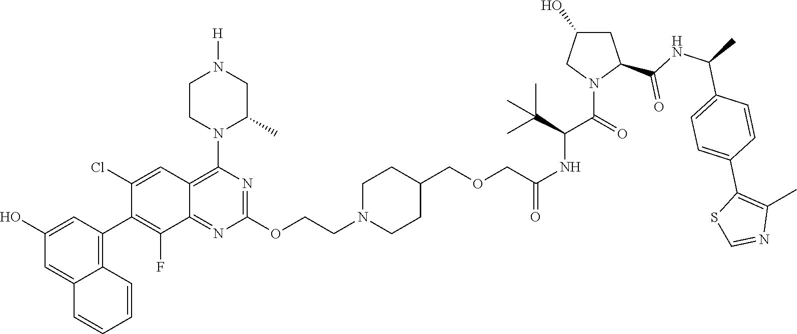 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCNC[C@@H]4C)c4cc(Cl)c(-c5cc(O)cc6ccccc56)c(F)c4n3)CC2)C(C)(C)C)cc1]

To a solution of tert-butyl (3S)-4-[6-chloro-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)quinazolin-4-yl]-3-methyl-piperazine-1-carboxylate (200 mg, 0.172 mmol, 1 eq) in CH₂Cl₂ (3 mL) was added HCl (4N in dioxane, 429 uL, 10 eq), and the reaction mixture was stirred at 20° C. for 1 hour. The organic phase was separated and concentrated, and the resulting residue was purified by prep-HPLC (30-90% CH₃CN in water (10 mM NH₄HCO₃)) to afford (2S,4R)-1-[(2S)-2-[[2-[[1-[2-[6-chloro-8-fluoro-7-(3-hydroxy-1-naphthyl)-4-[(2S)-2-methylpiperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (200 mg, 152.16 umol, 89% yield) as a white solid. LC/MS (ESI) m/z: 1065.9 [M+H]⁺.

### Step 10: Preparation of (2S,4R)-1-[(2S)-2-[[2-[[1-[2-[6-chloro-8-fluoro-7-(3-hydroxy-1-naphthyl)-4-[(2S)-2-methyl-4-prop-2-enoyl-piperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

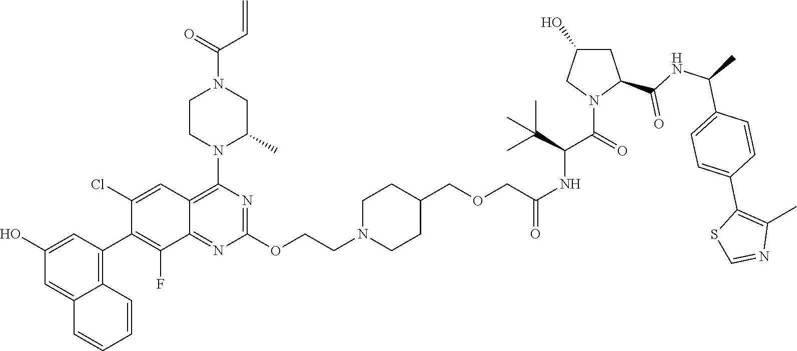 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)[C@@H](C)C1]

To a mixture of (2S,4R)-1-[(2S)-2-[[2-[[1-[2-[6-chloro-8-fluoro-7-(3-hydroxy-1-naphthyl)-4-[(2S)-2-methylpiperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (200 mg, 0.169 mmol, 90% purity, 1 eq) and 2,6-dimethylpyridine (91 mg, 0.845 mmol, 5 eq) in CH₂Cl₂ (4 mL) at −78° C. was added prop-2-enoyl chloride (15 mg, 0.169 mmol, 1 eq) dropwise, and the reaction mixture was stirred at −78° C. for 0.5 hours. The mixture was quenched with water (1 mL) at −78° C., then concentrated under reduced pressure. The resulting residue was purified by prep-HPLC (44-74% CH₃CN in water (10 mM NH₄HCO₃)) to afford (2S,4R)-1-[(2S)-2-[[2-[[1-[2-[6-chloro-8-fluoro-7-(3-hydroxy-1-naphthyl)-4-[(2S)-2- methyl-4-prop-2-enoyl-piperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (49 mg, 0.044 mmol, 26% yield) as a white solid. LC/MS (ESI) m/z: 1118.8 [M+H]⁺. ¹H-NMR (400 MHz, DMSO-d₆) δ 10.01 (d, J=4.0 Hz, 1H), 8.99-8.95 (m, 1H), 8.43 (d, J=8.0 Hz, 1H), 7.91 (s, 1H), 7.80 (d, J=8.0 Hz, 1H), 7.45-7.40 (m, 3H), 7.35-7.32 (m, 2H), 7.29-7.25 (m, 2H), 7.20 (d, J=4.0 Hz, 2H), 7.05 (d, J=4.0, 1H), 6.90-6.77 (m, 1H), 6.23-6.14 (m, 1H), 5.77-5.72 (m, 1H), 5.12 (d, J=4.0 Hz, 1H), 4.86 (s, 1H), 4.52 (d, J=8.0 Hz, 1H), 4.48-4.34 (m, 4H), 4.30-4.08 (m, 4H), 3.90 (s, 2H), 3.72-3.61 (m, 2H), 3.59-3.50 (m, 3H), 3.02-2.88 (m, 2H), 2.72-2.65 (m, 2H), 2.44 (s, 4H), 2.12-1.92 (m, 3H), 1.67-1.48 (m, 3H), 1.40-1.28 (m, 7H), 1.26-1.07 (m, 3H), 0.91 (s, 9H).

### Exemplary Synthesis of (2S,4R)-1-[(2R)-2-[3-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[(2R)-2-[6-Chloro-4-[(3S)-3-(cyanomethyl)-4-prop-2-enoyl- piperazin-1-yl]-8-fluoro-7-(3-hydroxy-1-naphthyl)quinazolin-2-yl]oxypropyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2R)-2-[3-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from 2-[(2S)-4-[6-chloro-8-fluoro-7-(3-hydroxy-1-naphthyl)-2-[(1R)-1-methyl-2-oxo-ethoxy]quinazolin-4-yl]piperazin-2-yl]acetonitrile and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide.

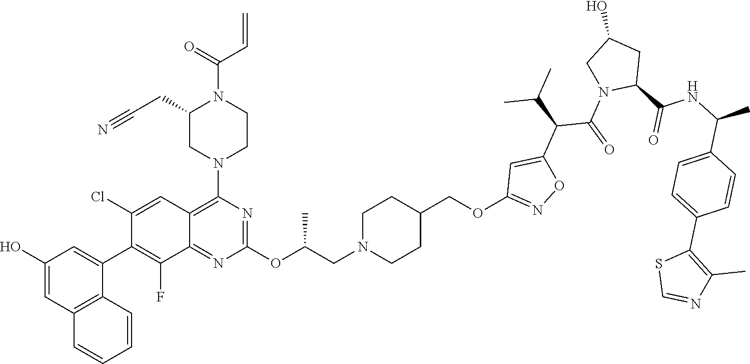 [SMILES: C=CC(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

(formic acid salt, white solid). LC/MS (ESI) m/z: 1153.7 [M+H]⁺. ¹H-NMR (400 MHz, DMSO-d₆) δ 8.99 (s, 1H), 8.42 (d, J=8.0 Hz, 1H), 8.17 (s, 1H), 8.13-8.09 (m, 1H), 7.82 (d, J=8.8 Hz, 1H), 7.48-7.41 (m, 2H), 7.39-7.33 (m, 2H), 7.29 (s, 1H), 7.25-7.15 (m, 2H), 7.10-7.05 (m, 1H), 6.97-6.77 (m, 1H), 6.26-6.17 (m, 1H), 6.04 (d, J=5.6 Hz, 1H), 5.89-5.77 (m, 1H), 5.47-5.34 (m, 1H), 5.14-4.97 (m, 1H), 4.96-4.84 (m, 1H), 4.84-4.81 (m, 1H), 4.43-4.17 (m, 5H), 4.14-4.03 (m, 1H), 3.97-3.82 (m, 3H), 3.73-3.59 (m, 4H), 3.11-2.91 (m, 5H), 2.70-2.61 (m, 1H), 2.47 (s, 3H), 2.29-2.15 (m, 2H), 2.07-1.95 (m, 4H), 1.77 (m, 1H), 1.72-1.56 (m, 4H), 1.40-1.30 (m, 6H), 1.22-1.10 (m, 2H), 0.99-0.91 (m, 3H), 0.83-0.74 (m, 3H).

### Exemplary Synthesis of (2S,4R)-1-[(2S)-2-[[2-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[(2R)-2-[6-Chloro-4-[(3S)-3-(cyanomethyl)-4-prop-2-enoyl- piperazin-1-yl]-8-fluoro-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in a analogous manner to (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[(2S)-4-[6-chloro-8-fluoro-7-(3-hydroxy-1-naphthyl)-2-[(1R)-1-methyl-2-oxo-ethoxy]quinazolin-4-yl]piperazin-2-yl]acetonitrile and (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide.

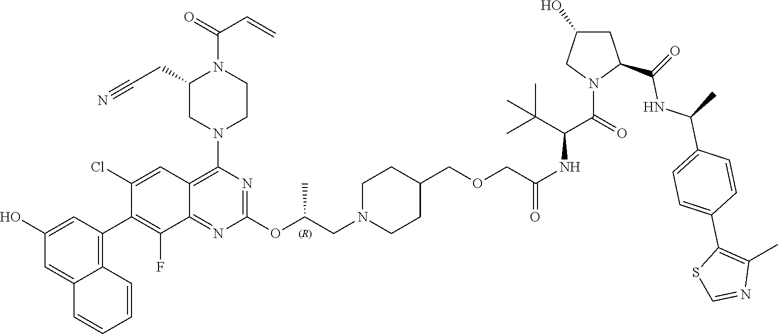

(free base, white solid). LC/MS (ESI) m/z: 1157.7 [M+H]⁺. ¹H-NMR (400 MHz, CD₃OD) δ 8.92-8.83 (m, 1H), 8.07 (s, 1H), 7.74 (d, J=8.4 Hz, 1H), 7.48-7.33 (m, 4H), 7.28-7.15 (m, 3H), 7.03 (s, 1H), 6.98-6.74 (m, 1H), 6.31 (d, J=17.2 Hz, 1H), 5.90-5.79 (m, 1H), 5.70-5.52 (m, 1H), 5.17-4.94 (m, 1H), 4.85-4.80 (m, 1H), 4.69-4.52 (m, 5H), 4.50-4.40 (m, 3H), 4.22-4.09 (m, 1H), 3.96-3.80 (m, 4H), 3.77-3.63 (m, 2H), 3.59-3.47 (m, 1H), 3.17-2.96 (m, 4H), 2.85-2.70 (m, 1H), 2.59-2.51 (m, 1H), 2.47 (s, 3H), 2.26-2.15 (m, 2H), 2.14-2.07 (m, 1H), 2.01-1.90 (m, 1H), 1.77-1.59 (m, 3H), 1.57-1.44 (m, 3H), 1.39 (d, J=6.0 Hz, 3H), 1.27-1.12 (m, 2H), 1.01 (s, 9H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-fluoro-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-fluoro-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl- piperazin-1-yl]-8-fluoro-7-(3-hydroxy-1-naphthyl)quinazolin-2-yl]oxyethyl]-4-methyl-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 4-fluoro-4-(hydroxymethyl)piperidine-1-carboxylate.

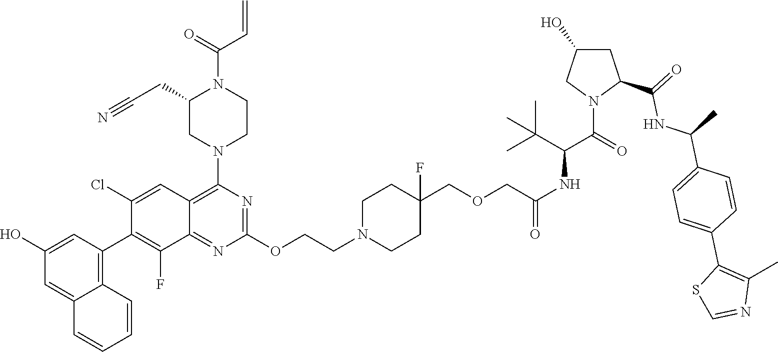 [SMILES: C=CC(=O)N1CCN(c2nc(OCCN3CCC(F)(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(formic acid salt, white solid). LC/MS (ESI) m/z: 1161.4 [M+H]⁺. ¹H-NMR (400 MHz, DMSO-d₆) δ 10.12-9.95 (m, 1H), 8.98 (s, 1H), 8.44 (d, J=8.0 Hz, 1H), 8.18-8.10 (m, 1H), 7.81 (d, J=8.4 Hz, 2H), 7.45-7.35 (m, 5H), 7.30-7.21 (m, 3H), 7.07 (d, J=2.0 Hz, 1H), 6.21 (d, J=16.4 Hz, 1H), 5.79 (d, J=10.0 Hz, 1H), 5.20-4.76 (m, 4H), 4.56-4.40 (m, 5H), 4.38-4.17 (m, 4H), 3.99 (s, 2H), 3.62-3.49 (m, 6H), 2.75-2.74 (s, 8H), 2.45 (s, 3H), 2.36-2.29 (m, 2H), 2.10-2.02 (m, 1H), 1.82-1.68 (m, 4H), 1.35 (d, J=6.8 Hz, 3H), 0.92 (s, 9H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-[[(2-ethoxy-2-oxo-ethyl)-methyl-amino]methyl]piperidine-1-carboxylate

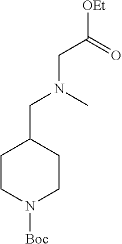 [SMILES: CCOC(=O)CN(C)CC1CCN(C(=O)OC(C)(C)C)CC1]

To a solution of tert-butyl 4-(methylaminomethyl)piperidine-1-carboxylate (1.00 g, 4.38 mmol, 1 eq) in CH₃CN (10 mL) were added K₂CO₃ (908 mg, 6.57 mmol, 1.5 eq) and ethyl 2-bromoacetate (731 mg, 4.38 mmol, 1 eq), and the reaction mixture was stirred at 80° C. under N₂ for 4 hours. The reaction mixture was filtered, and the filtrate was concentrated under reduced pressure. The resulting crude product was purified by flash silica gel chromatography (gradient: 0-30% EtOAc in petroleum ether) to give tert-butyl 4-[[(2-ethoxy-2-oxo-ethyl)-methyl-amino]methyl]piperidine-1-carboxylate (1.18 g, 3.75 mmol, 86% yield) as a colorless oil. ¹H-NMR (400 MHz, CDCl₃) δ 4.19 (q, J=7.2 Hz, 2H), 4.14-3.97 (m, 2H), 3.28 (s, 2H), 2.80-2.60 (m, 2H), 2.55-2.25 (m, 5H), 1.82-1.63 (m, 3H), 1.46 (s, 9H), 1.29 (t, J=7.2 Hz, 3H), 1.16-1.01 (m, 2H).

### Step 2: Preparation of 2-[(1-tert-butoxycarbonyl-4-piperidyl)methyl-methyl-amino]acetic Acid

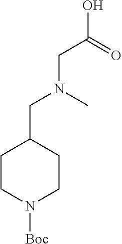 [SMILES: CN(CC(=O)O)CC1CCN(C(=O)OC(C)(C)C)CC1]

To a solution of tert-butyl 4-[[(2-ethoxy-2-oxo-ethyl)-methyl-amino]methyl]piperidine-1-carboxylate (1.18 g, 3.75 mmol, 1 eq) in THF (6 mL) was added a solution of LiOH.H₂O (315 mg, 7.51 mmol, 2 eq) in water (6 mL), and the reaction mixture was stirred at 25° C. under N₂ for 1 hours. The reaction mixture was concentrated under reduced pressure, and the resulting residue was diluted with water (10 mL). The pH of the aqueous mixture was adjusted to ˜5 by addition of HCl (2N in water). Lyophilization afforded crude 2-[(1-tert-butoxycarbonyl-4-piperidyl)methyl-methyl-amino]acetic acid (1.01 g) as a white solid.

### Step 3: Preparation of tert-butyl 4-[[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-methyl-amino]methyl]piperidine-1-carboxylate

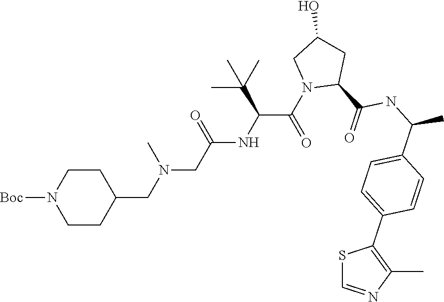

To a solution of 2-[(1-tert-butoxycarbonyl-4-piperidyl)methyl-methyl-amino]acetic acid (500 mg, 1.75 mmol, 1 eq) in CH₂Cl₂ (15 mL) were added diisopropylethylamine (1.13 g, 8.73 mmol, 1.52 mL, 5 eq) and HATU (797 mg, 2.10 mmol, 1.2 eq), and the resulting mixture was stirred at 25° C. under N₂ for 10 minutes. (2S,4R)-1-[(2S)-2-Amino-3,3-dimethyl-butanoyl]-4-hydroxy-N-[(1S)-1-[4-(4-methylthiazol-5-yl)phenyl]ethyl]pyrrolidine-2-carboxamide (840 mg, 1.75 mmol, 1 eq, HCl) was then added, and the reaction mixture was stirred at 25° C. under N₂ for 0.5 hour. The reaction mixture was poured onto water (20 mL) and the layers were separated. The aqueous layer was extracted with CH₂Cl₂ (20 mL), and the combined organic extract was washed with brine (20 mL), dried over anhydrous Na₂SO₄, filtered, and concentrated. The resulting crude product was purified by flash silica gel chromatography (gradient: 0-100% EtOAc in petroleum ether to 10% CH₃OH in CH₂Cl₂) to give tert-butyl 4-[[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-methyl-amino]methyl]piperidine-1-carboxylate (922 mg, 1.29 mmol, 74% yield) as a white solid. LC/MS (ESI) m/z: 357.4 [M/2+H]⁺.

### Step 4: Preparation of (2S,4R)-1-[(2S)-3,3-dimethyl-2-[[2-[methyl(4-piperidylmethyl)amino]acetyl]amino]butanoyl]-4-hydroxy-N-[(1S)-1-[4-(4-methylthiazol-5-yl)phenyl]ethyl]pyrrolidine-2-carboxamide

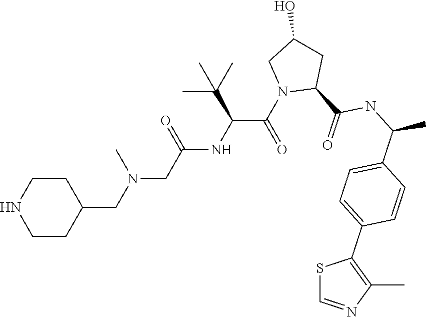

A mixture of tert-butyl 4-[[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-methyl-amino]methyl]piperidine-1-carboxylate (300 mg, 0.421 mmol, 1 eq) in CH₃OH (2 mL) and HCl (4N in dioxane, 5 mL) was stirred at 25° C. for 1 hour. The reaction mixture was concentrated to give a residue which was dissolved in water (3 mL). The pH of the aqueous mixture was adjusted to ˜8 by addition of saturated aqueous NaHCO₃, and the resulting basic mixture was concentrated under reduced pressure to give a residue. CH₂Cl₂/CH₃OH (33 mL, 10/1) was then added, the resulting suspension was filtered, and the filtrate was concentrated under reduced pressure to give (2S,4R)-1-[(2S)-3,3-dimethyl-2-[[2-[methyl(4-piperidylmethyl)amino]acetyl]amino]butanoyl]-4-hydroxy-N-[(1S)-1-[4-(4-methylthiazol-5-yl)phenyl]ethyl]pyrrolidine-2-carboxamide (250 mg, 0.408 mmol, 97% yield) as a white solid. LC/MS (ESI) m/z: 307.3 [M/2+H]⁺.

### Step 5: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[(2S)-4-[6-chloro-8-fluoro-7-(3-hydroxy-1-naphthyl)-2-[(1R)-1-methyl-2-oxo-ethoxy]quinazolin-4-yl]piperazin-2-yl]acetonitrile starting from (2S,4R)-1-[(2S)-3,3-dimethyl-2-[[2-[methyl(4-piperidylmethyl)amino]acetyl]amino]butanoyl]-4-hydroxy-N-[(1S)-1-[4-(4-methylthiazol-5-yl)phenyl]ethyl]pyrrolidine-2-carboxamide and 2-[(2S)-4-[6-chloro-8-fluoro-7-(3-hydroxy-1-naphthyl)-2-(2-oxoethoxy)quinazolin-4-yl]piperazin-2-yl]acetonitrile.

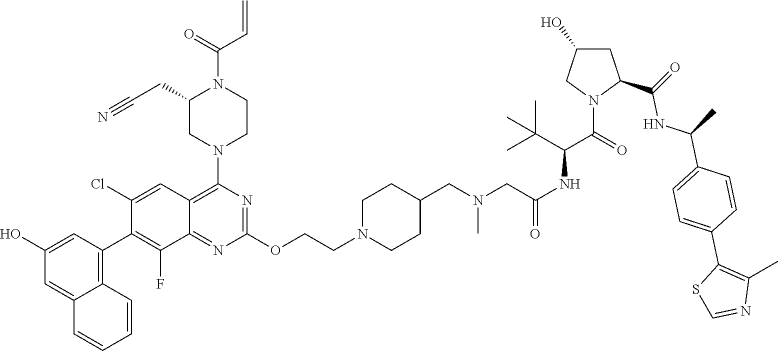 [SMILES: C=CC(=O)N1CCN(c2nc(OCCN3CCC(CN(C)CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(formic acid salt, white solid). LC/MS (ESI) m/z: 1156.8 [M+H]⁺. ¹H-NMR (400 MHz, CD₃OD) δ 8.90-8.80 (m, 1H), 8.53 (s, 1H), 8.08 (s, 1H), 7.79-7.71 (m, 1H), 7.47-7.32 (m, 5H), 7.29-7.12 (m, 3H), 7.06-7.01 (m, 1H), 6.97-6.72 (m, 1H), 6.31 (d, J=16.8 Hz, 1H), 5.85 (d, J=10.0 Hz, 1H), 4.56-4.39 (m, 4H), 4.26-4.02 (m, 1H), 3.86-3.81 (m, 1H), 3.76-3.70 (m, 1H), 3.59-3.37 (m, 3H), 3.14-2.96 (m, 4H), 2.70-2.59 (m, 1H), 2.49-2.41 (m, 3H), 2.34-2.17 (m, 5H), 2.03-1.86 (m, 3H), 1.77-1.64 (m, 1H), 1.58-1.28 (m, 6H), 1.09-0.93 (m, 9H).

### Exemplary Synthesis of (2S,4R)-1-[(2S)-2-[[2-[7-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-2,7-diazaspiro[3.5]nonan-2-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 2-(2-ethoxy-2-oxo-ethyl)-2,7-diazaspiro[3.5]nonane-7-carboxylate

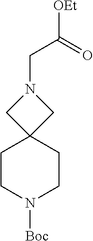 [SMILES: CCOC(=O)CN1CC2(CCN(C(=O)OC(C)(C)C)CC2)C1]

To a mixture of tert-butyl 2,7-diazaspiro[3.5]nonane-7-carboxylate (500 mg, 2.21 mmol, 1 eq) and ethyl 2-oxoacetate (451 mg, 2.21 mmol, 50% purity, 1 eq) in CH₃OH (3 mL) were added acetic acid (531 mg, 8.84 mmol, 0.505 mL, 4 eq) and 2-picolineborane complex (1.18 g, 11.1 mmol, 5 eq), and the reaction mixture was stirred at 25° C. for 1 hour. The reaction was concentrated, and the resulting residue was purified by flash silica gel chromatography (gradient: 0-30% THF in petroleum ether) to give tert-butyl 2-(2-ethoxy-2-oxo-ethyl)-2,7-diazaspiro[3.5]nonane-7-carboxylate (350 mg, 1.01 mmol, 46% yield) as a colorless oil. LC/MS (ESI) m/z: 313.3 [M+H]⁺.

### Step 2: Preparation of 2-(7-tert-butoxycarbonyl-2,7-diazaspiro[3.5]nonan-2-yl)acetic Acid

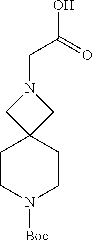 [SMILES: CC(C)(C)OC(=O)N1CCC2(CC1)CN(CC(=O)O)C2]

To tert-butyl 2-(2-ethoxy-2-oxo-ethyl)-2,7-diazaspiro[3.5]nonane-7-carboxylate (350 mg, 1.12 mmol, 1 eq) in THF (5 mL) and H₂O (5 mL) was added LiOH.H₂O (235 mg, 5.60 mmol, 5 eq), and the reaction mixture was stirred at 25° C. for 1 hour. The reaction was concentrated to remove THF, and 2N aqueous HCl was then added until pH=5. The mixture was lyophilized to afford 2-(7-tert-butoxycarbonyl-2,7-diazaspiro[3.5]nonan-2-yl)acetic acid (318 mg, crude) as white solid. LC/MS (ESI) m/z: 285.2 [M+H]⁺.

### Step 3: Preparation of tert-butyl 2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-2,7-diazaspiro[3.5]nonane-7-carboxylate

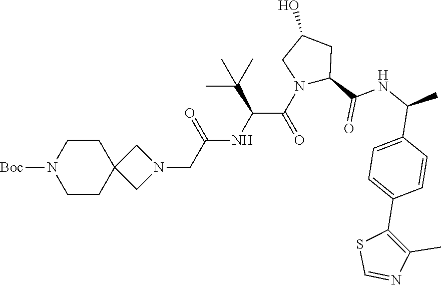 [SMILES: Cc1ncsc1-c1ccc([C@H](C)NC(=O)[C@@H]2C[C@@H](O)CN2C(=O)[C@@H](NC(=O)CN2CC3(CCN(C(=O)OC(C)(C)C)CC3)C2)C(C)(C)C)cc1]

To a solution of 2-(7-tert-butoxycarbonyl-2,7-diazaspiro[3.5]nonan-2-yl)acetic acid (318 mg, 1.12 mmol, 1 eq) in DMF (10 mL) were added HATU (638 mg, 1.68 mmol, 1.5 eq) and diisopropylethylamine (723 mg, 5.59 mmol, 0.974 mL, 5 eq) followed by (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (538 mg, crude, HCl), and the reaction mixture was stirred at 25° C. for 1 hour. Water (10 mL) was then added, and the resulting mixture was extracted with CH₂Cl₂ (3×20 mL). The combined organic extract was washed with brine (3×20 mL), dried over anhydrous Na₂SO₄, filtered, and concentrated. The resulting crude product was purified by flash silica gel chromatography (gradient: 0-100% EtOAc in petroleum ether followed by 10% CH₃OH in CH₂Cl₂) to give tert-butyl 2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-2,7-diazaspiro[3.5]nonane-7-carboxylate (640 mg, 0.729 mmol, 65% yield) as yellow solid. LC/MS (ESI) m/z: 711.3 [M+H]⁺.

### Step 4: Preparation of (2S,4R)-1-[(2S)-2-[[2-(2,7-diazaspiro[3.5]nonan-2-yl)acetyl]amino]-3,3-dimethyl-butanoyl]-4-hydroxy-N-[(1S)-1-[4-(4-methylthiazol-5-yl)phenyl]ethyl]pyrrolidine-2-carboxamide

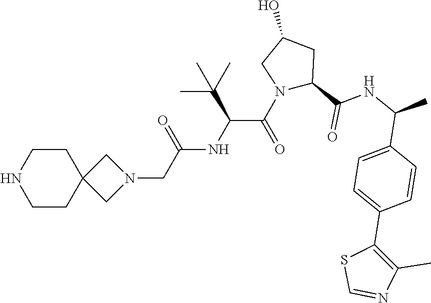 [SMILES: Cc1ncsc1-c1ccc([C@H](C)NC(=O)[C@@H]2C[C@@H](O)CN2C(=O)[C@@H](NC(=O)CN2CC3(CCNCC3)C2)C(C)(C)C)cc1]

To a solution of tert-butyl 2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-2,7-diazaspiro[3.5]nonane-7-carboxylate (640 mg, 900.24 umol, 1 eq) in CH₃OH (3 mL) was added HCl (4N in dioxane, 3 mL), and the reaction mixture was stirred at 25° C. for 1 hour. The reaction mixture was concentrated, and the resulting residue was diluted with water (10 mL). Saturated aqueous NaHCO3 was added to the aqueous mixture until pH=8. Lyophilization afforded (2S,4R)-1-[(2S)-2-[[2-(2,7-diazaspiro[3.5]nonan-2-yl)acetyl]amino]-3,3-dimethyl-butanoyl]-4-hydroxy-N-[(1S)-1-[4-(4-methylthiazol-5-yl)phenyl]ethyl]pyrrolidine-2-carboxamide (550 mg, crude) as a yellow solid. LC/MS (ESI) m/z: 306.4 [M/2+H]⁺.

### Step 5: Preparation of (2S,4R)-1-[(2S)-2-[[2-[7-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-2,7-diazaspiro[3.5]nonan-2-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[7-[2-[6-Chloro-4-[(3S)-3-(cyanomethyl)-4-prop-2-enoyl-piperazin-1- yl]-8-fluoro-7-(3-hydroxy-1-naphthyl)quinazolin-2-yl]oxyethyl]-2,7-diazaspiro[3.5]nonan-2-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from (2S,4R)-1-[(2S)-2-[[2-(2,7-diazaspiro[3.5]nonan-2-yl)acetyl]amino]-3,3-dimethyl-butanoyl]-4-hydroxy-N-[(1S)-1-[4-(4-methylthiazol-5-yl)phenyl]ethyl]pyrrolidine-2-carboxamide and 2-[(2S)-4-[6-chloro-8-fluoro-7-(3-hydroxy-1-naphthyl)-2-(2-oxoethoxy)quinazolin-4-yl]piperazin-2-yl]acetonitrile.

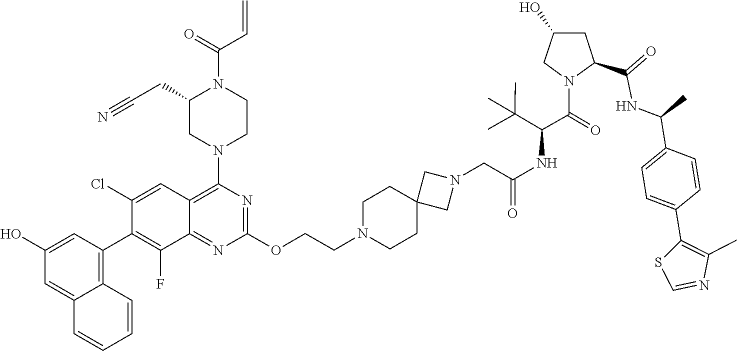 [SMILES: C=CC(=O)N1CCN(c2nc(OCCN3CCC4(CC3)CN(CC(=O)N[C@H](C(=O)N3C[C@H](O)C[C@H]3C(=O)N[C@@H](C)c3ccc(-c5scnc5C)cc3)C(C)(C)C)C4)nc3c(F)c(-c4cc(O)cc5ccccc45)c(Cl)cc23)C[C@@H]1CC#N]

(free base, white solid). LC/MS (ESI) m/z: 1154.7 [M+H]⁺. ¹H-NMR (400 MHz, CD₃OD) δ 8.88-8.83 (m, 1H), 8.03 (s, 1H), 7.74 (d, J=8.4 Hz, 1H), 7.43-7.36 (m, 5H), 7.28-7.17 (m, 3H), 7.07-7.01 (m, 1H), 6.88-6.72 (m, 1H), 6.30 (d, J=16.8 Hz, 1H), 5.84 (d, J=10.0 Hz, 1H), 5.05-4.94 (m, 3H), 4.58-4.36 (m, 7H), 3.90-3.49 (m, 6H), 3.21-3.13 (m, 6H), 3.02 (s, 2H), 2.81 (t, J=5.6 Hz, 2H), 2.54 (s, 2H), 2.47 (s, 3H), 2.24-2.15 (m, 1H), 1.97-1.88 (m, 1H), 1.81 (s, 4H), 1.59-1.30 (m, 4H), 1.05-1.00 (m, 9H).

### Exemplary Synthesis of (2S,4R)-1-[(2R)-2-[3-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

(2S,4R)-1-[(2R)-2-[3-[[1-[2-[6-Chloro-8-fluoro-7-(3-hydroxy-1-naphthyl)-4-(4-prop-2-enoylpiperazin-1-yl)quinazolin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[2-[[1-[2-[6-chloro-8-fluoro-7-(3-hydroxy-1-naphthyl)-4-(4-prop-2-enoylpiperazin-1-yl)quinazolin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from tert-butyl 4-[6-chloro-8-fluoro-7-(3-hydroxy-1-naphthyl)-2-(2-oxoethoxy)quinazolin-4-yl]piperazine-1-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide.

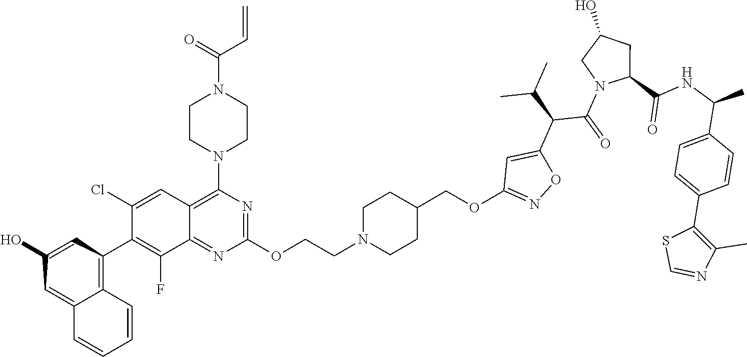

(formic acid salt, off-white solid). LC/MS (ESI) m/z: 1100.4 [M+H]⁺. ¹H-NMR (400 MHz, CD₃OD) δ 8.89-8.86 (m, 1H), 8.50 (s, 1H), 8.05 (s, 1H), 7.75 (d, J=8.4 Hz, 1H), 7.48-7.34 (m, 5H), 7.29-7.14 (m, 3H), 7.04 (d, J=2.4 Hz, 1H), 6.83 (dd, J=16.4, 10.4 Hz, 1H), 6.29 (dd, J=16.4, 1.6 Hz, 1H), 6.00-5.92 (m, 1H), 5.82 (dd, J=10.8, 2.0 Hz, 1H), 4.54-4.33 (m, 3H), 4.10-4.02 (m, 6H), 3.97-3.89 (m, 4H), 3.83 (dd, J=10.8, 4.0 Hz, 1H), 3.67 (d, J=10.0 Hz, 1H), 3.61 (d, J=10.8 Hz, 1H), 3.56-3.35 (m, 3H), 3.25-3.17 (m, 2H), 2.81-2.55 (m, 3H), 2.51-2.43 (m, 3H), 2.40-2.31 (m, 1H), 2.23-2.13 (m, 1H), 2.03-1.87 (m, 4H), 1.60-1.48 (m, 5H), 1.05 (d, J=6.4 Hz, 3H), 0.88 (d, J=6.4 Hz, 3H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-fluoro-4-piperidyl]methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-[[(2-benzyloxy-2-oxo-ethyl)amino]methyl]-4-fluoro-piperidine-1-carboxylate

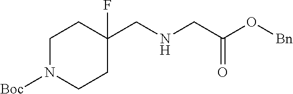 [SMILES: CC(C)(C)OC(=O)N1CCC(F)(CNCC(=O)OCc2ccccc2)CC1]

To a solution of tert-butyl 4-(aminomethyl)-4-fluoro-piperidine-1-carboxylate (2.00 g, 8.61 mmol, 1 eq) and benzyl 2-bromoacetate (1.99 g, 8.67 mmol, 1.01 eq) in CH₂Cl₂ (20 mL) was added K₂CO₃ (3.57 g, 25.8 mmol, 3 eq), and the reaction mixture was stirred at 25° C. for 10 hours. The mixture was filtered, and the filtrate was concentrated under reduced pressure. The resulting crude product was purified by flash silica gel chromatography (gradient: 0-15% THF in petroleum ether) to give tert-butyl 4-[[(2-benzyloxy-2-oxo-ethyl)amino]methyl]-4-fluoro-piperidine-1-carboxylate (2.30 g, 6.05 mmol, 70% yield) as a yellow oil. LC/MS (ESI) m/z: 381.1 [M+H]⁺.

### Step 2: Preparation of tert-butyl 4-[[(2-benzyloxy-2-oxo-ethyl)-methyl-amino]methyl]-4-fluoro-piperidine-1-carboxylate

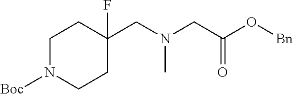 [SMILES: CN(CC(=O)OCc1ccccc1)CC1(F)CCN(C(=O)OC(C)(C)C)CC1]

To a mixture of tert-butyl 4-[[(2-benzyloxy-2-oxo-ethyl)amino]methyl]-4-fluoro-piperidine-1-carboxylate (2.30 g, 6.05 mmol, 1 eq) and HCHO (1.47 g, 18.1 mmol, 1.35 mL, 37% purity, 3 eq) in CH₃OH (15 mL) and CH₂Cl₂ (15 mL) at 25° C. were added AcOH (545 mg, 9.07 mmol, 1.5 eq) and NaBH(OAc)₃ (5.13 g, 24.2 mmol, 4 eq) in one portion, and the reaction mixture was stirred at 25° C. for 10 hours. The mixture was quenched with saturated Na₂CO₃ solution (20 mL) and stirred for 30 minutes. The resulting mixture was extracted with CH₂Cl₂ (3×20 mL), and the combined extracts were dried over anhydrous Na₂SO₄, filtered, and concentrated. The crude product was purified by flash silica gel chromatography (gradient: 0-15% THF in petroleum ether) to give tert-butyl 4-[[(2-benzyloxy-2-oxo-ethyl)-methyl-amino]methyl]-4-fluoro-piperidine-1-carboxylate (1.70 g, 4.31 mmol, 71% yield) as a colorless oil. LC/MS (ESI) m/z: 395.2 [M+H]⁺.

### Step 3: Preparation of 2-[(1-tert-butoxycarbonyl-4-fluoro-4-piperidyl)methyl-methyl-amino]acetic Acid

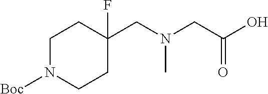 [SMILES: CN(CC(=O)O)CC1(F)CCN(C(=O)OC(C)(C)C)CC1]

To a solution of tert-butyl 4-[[(2-benzyloxy-2-oxo-ethyl)-methyl-amino]methyl]-4-fluoro-piperidine-1-carboxylate (1.70 g, 4.31 mmol, 1 eq) in THF (2 mL), CH₃OH (2 mL), and H₂O (2 mL) was added LiOH.H₂O (542 mg, 12.9 mmol, 3 eq), and the reaction mixture was stirred at 25° C. for 1 hour. Water (10 mL) was then added, and the resulting mixture was extracted with EtOAc (3×10 mL). The pH of the aqueous phase was adjusted to ˜5 by addition of 2N aqueous HCl, and the resulting acidic mixture was lyophilized. The crude product was triturated with CH₂Cl₂ (20 mL). The resulting suspension was filtered, and the filtrate was concentrated under reduced pressure to give 2-[(l-tert-butoxycarbonyl-4-fluoro-4-piperidyl)methyl-methyl-amino]acetic acid (1.1 g, crude) as a white solid. confirmed by LCMS:EB777-592-P1A1, which was used to next step without purification. LC/MS (ESI) m/z: 305.1 [M+H]⁺.

### Step 4: Preparation of tert-butyl 4-fluoro-4-[[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-methyl-amino]methyl]piperidine-1-carboxylate

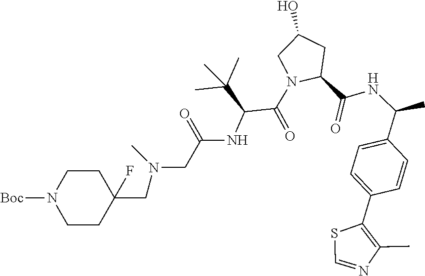 [SMILES: Cc1ncsc1-c1ccc([C@H](C)NC(=O)[C@@H]2C[C@@H](O)CN2C(=O)[C@@H](NC(=O)CN(C)CC2(F)CCN(C(=O)OC(C)(C)C)CC2)C(C)(C)C)cc1]

To a solution of 2-[(1-tert-butoxycarbonyl-4-fluoro-4-piperidyl)methyl-methyl-amino]acetic acid (500 mg, 1.64 mmol, 1 eq) in DMF (7 mL) were added diisopropylethylamine (1.08 g, 8.32 mmol, 1.45 mL, 5.07 eq) and HATU (937 mg, 2.46 mmol, 1.5 eq) followed by (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (790 mg, 1.64 mmol, 1 eq, HCl), and the reaction mixture was stirred at 20° C. for 1 hour. The mixture was diluted with water (20 mL) and extracted with CH₂Cl₂ (3×20 mL). The combined organic extracts were washed with brine (30 mL×3), dried over anhydrous Na₂SO₄, filtered, and concentrated. The resulting crude product was purified by flash silica gel chromatography (gradient: 0-60% THF in petroleum ether) to give tert-butyl 4-fluoro-4-[[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-methyl-amino]methyl]piperidine-1-carboxylate (1.00 g, 1.37 mmol, 83% yield) as a white solid. . LC/MS (ESI) m/z: 731.4 [M+H]⁺.

### Step 5: Preparation of (2S,4R)-1-[(2S)-2-[[2-[(4-fluoro-4-piperidyl)methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide

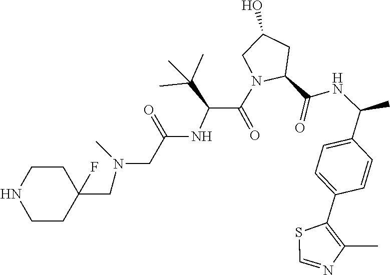 [SMILES: Cc1ncsc1-c1ccc([C@H](C)NC(=O)[C@@H]2C[C@@H](O)CN2C(=O)[C@@H](NC(=O)CN(C)CC2(F)CCNCC2)C(C)(C)C)cc1]

A solution of tert-butyl 4-fluoro-4-[[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-methyl-amino]methyl]piperidine-1-carboxylate (600 mg, 0.821 mmol, 1 eq) in HCl (6 mL, 4N in dioxane) was stirred at 20° C. for 1 hour. The reaction mixture was concentrated at 30° C., and the resulting residue was diluted with water (10 mL). The pH of the aqueous mixture was adjusted to ˜8 by addition of aqueous Na₂CO₃, and the resulting basic mixture was extracted with 10:1 CH₂Cl₂/CH₃OH (3×20 mL). The combined organic extracts were washed with brine (3×30 mL), dried over anhydrous Na₂SO₄, filtered, and concentrated to give (2S,4R)-1-[(2S)-2-[[2-[(4-fluoro-4-piperidyl)methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide (490 mg, crude). LC/MS (ESI) m/z: 631.1 [M+H]⁺.

### Step 6: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-fluoro-4-piperidyl]methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-fluoro-4-piperidyl]methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from (2S,4R)-1-[(2S)-2-[[2-[(4-fluoro-4-piperidyl)methyl-methyl-amino]acetyl]amino]-3,3-dimethyl-butanoyl]-4-hydroxy-N-[(1S)-1-[4-(4-methylthiazol-5-yl)phenyl]ethyl]pyrrolidine-2-carboxamide and 2-[(2S)-4-[6-chloro-8-fluoro-7-(3-hydroxy-1-naphthyl)-2-(2-oxoethoxy)quinazolin-4-yl]piperazin-2-yl]acetonitrile.

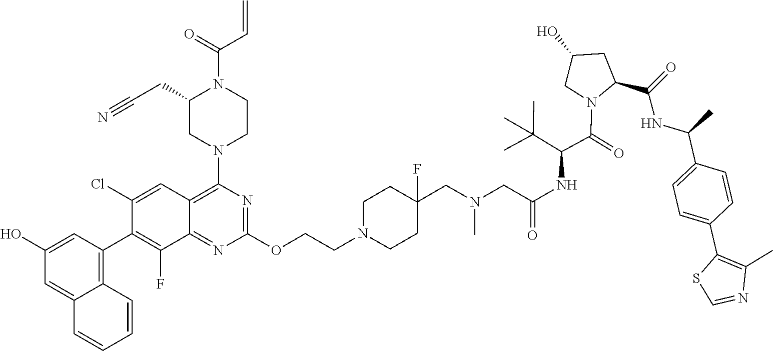 [SMILES: C=CC(=O)N1CCN(c2nc(OCCN3CCC(F)(CN(C)CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N]

(formic acid salt, white solid). LC/MS (ESI) m/z: 1174.4 [M+H]⁺. ¹H-NMR (400 MHz, CD₃OD) δ 8.97-8.70 (m, 1H), 8.39 (s, 1H), 8.08 (s, 1H), 7.74 (d, J=8.0 Hz, 1H), 7.45-7.31 (m, 5H), 7.30-7.12 (m, 3H), 7.07-6.98 (m, 1H), 6.93-6.72 (m, 1H), 6.31 (d, J=16.8 Hz, 1H), 5.84 (d, J=10.4 Hz, 1H), 5.13-4.90 (m, 2H), 4.75-4.36 (m, 7H), 4.12-3.43 (m, 6H), 3.28-2.62 (m, 12H), 2.46-2.34 (m, 6H), 2.26-1.78 (m, 6H), 1.45 (d, J=7.2 Hz, 3H), 1.06-0.97 (m, 9H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(5-hydroxy-2,3-dimethyl-phenyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 3,4-dimethyl-5-(4,4,5,5-tetramethyl-1,3,2-dioxaborolan-2-yl)phenol

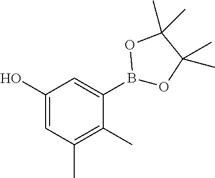 [SMILES: Cc1cc(O)cc(B2OC(C)(C)C(C)(C)O2)c1C]

To a solution of 3-bromo-4,5-dimethyl-phenol (200 mg, 0.995 mol, 1 eq) and 4,4,5,5-tetramethyl-2-(4,4,5,5-tetramethyl-1,3,2-dioxaborolan-2-yl)-1,3,2-dioxaborolane (328 mg, 1.29 mmol, 1.3 eq) in dioxane (5 mL) were added KOAc (244 mg, 2.49 mmol, 2.5 eq) and Pd(dppf)C12 (73 mg, 0.099 mmol, 0.1 eq), and the reaction mixture was stirred at 90° C. under N₂ for 5 hours. The reaction mixture was concentrated under reduced pressure, and the resulting crude product was purified by flash silica gel chromatography (gradient: 0-5% THF in petroleum ether) to give 3,4-dimethyl-5-(4,4,5,5-tetramethyl-1,3,2-dioxaborolan-2-yl)phenol (207 mg, 0.834 mmol, 84% yield) as a yellow solid. LC/MS (ESI) m/z: 249.2 [M+H]⁺.

### Step 2: Preparation of tert-butyl (2S)-4-[6-chloro-2-[2-[4-[(2-ethoxy-2-oxo-ethoxy)methyl]-1-piperidyl]ethoxy]-8-fluoro-7-(5-hydroxy-2,3-dimethyl-phenyl)quinazolin-4-yl]-2-(cyanomethyl)piperazine-1-carboxylate

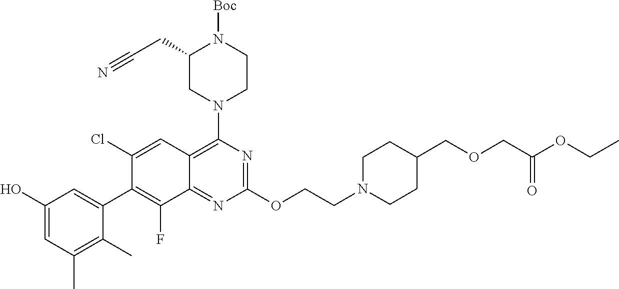 [SMILES: CCOC(=O)COCC1CCN(CCOc2nc(N3CCN(C(=O)OC(C)(C)C)[C@@H](CC#N)C3)c3cc(Cl)c(-c4cc(O)cc(C)c4C)c(F)c3n2)CC1]

To a solution of tert-butyl (2S)-4-[7-bromo-6-chloro-2-[2-[4-[(2-ethoxy-2-oxo-ethoxy)methyl]-1-piperidyl]ethoxy]-8-fluoro-quinazolin-4-yl]-2-(cyanomethyl)piperazine-1-carboxylate (100 mg, 0.137 mmol, 1.0 eq) and 3,4-dimethyl-5-(4,4,5,5-tetramethyl-1,3,2-dioxaborolan-2-yl)phenol (51 mg, 0.21 mmol, 1.5 eq) in dioxane (5 mL) were added K₃PO₄ (1.5 M, 0.275 mL, 3 eq) and XPhos Pd G3 (12 mg, 0.014 mmol, 0.1 eq), and the reaction mixture was stirred at 100° C. under N₂ for 16 hours. The reaction mixture was filtered, and the filtrate was dried over anhydrous Na₂SO₄, filtered, and concentrated. The resulting crude product was purified by flash silica gel chromatography (gradient: 0-50% THF in petroleum ether) to give tert-butyl (2S)-4-[6-chloro-2-[2-[4-[(2-ethoxy-2-oxo-ethoxy)methyl]-1-piperidyl]ethoxy]-8-fluoro-7-(5-hydroxy-2,3-dimethyl-phenyl)quinazolin-4-yl]-2-(cyanomethyl)piperazine-1-carboxylate (135 mg, 0.128 mmol, 93% yield) as a yellow solid. LC/MS (ESI) m/z: 769.3 [M+H]⁺.

### Step 3: Preparation of 2-[[1-[2-[4-[(3S)-4-tert-butoxycarbonyl-3-(cyanomethyl)piperazin-1-yl]-6-chloro-8-fluoro-7-(5-hydroxy-2,3-dimethyl-phenyl)quinazolin-2-yl]oxyethyl]-4-piperidyl]methoxy]acetic Acid

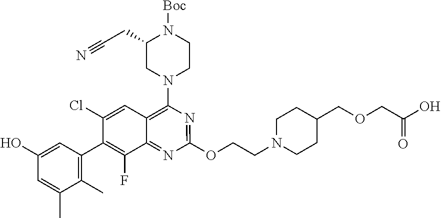 [SMILES: Cc1cc(O)cc(-c2c(Cl)cc3c(N4CCN(C(=O)OC(C)(C)C)[C@@H](CC#N)C4)nc(OCCN4CCC(COCC(=O)O)CC4)nc3c2F)c1C]

To a solution of tert-butyl (2S)-4-[6-chloro-2-[2-[4-[(2-ethoxy-2-oxo-ethoxy)methyl]-1-piperidyl]ethoxy]-8-fluoro-7-(5-hydroxy-2,3-dimethyl-phenyl)quinazolin-4-yl]-2-(cyanomethyl)piperazine-1-carboxylate (135 mg, 0.128 mmol, 73% purity, 1 eq) in THF (0.5 mL), water (0.5 mL), and EtOH (0.5 mL) was added LiOH.H₂O (27 mg, 0.64 mmol, 5 eq), and the reaction mixture was stirred at 20° C. under N₂ for 2 hours. The reaction mixture was concentrated under reduced pressure, and the resulting residue was dissolved in water (15 mL). The aqueous mixture was adjusted to pH=6 with 2N aqueous HCl and dried by lyophilization to give 2-[[1-[2-[4-[(3S)-4-tert-butoxycarbonyl-3-(cyanomethyl)piperazin-1-yl]-6-chloro-8-fluoro-7-(5-hydroxy-2,3-dimethyl-phenyl)quinazolin-2-yl]oxyethyl]-4-piperidyl]methoxy]acetic acid (90 mg, crude) as a yellow solid. LC/MS (ESI) m/z: 741.2 [M+H]⁺.

### Step 4: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(5-hydroxy-2,3-dimethyl-phenyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-prop-2-enoyl-piperazin-1-yl]-8-fluoro-7-(5-hydroxy-2,3-dimethyl-phenyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-8-fluoro-7-(3-hydroxy-1-naphthyl)-4-[(2S)-2-methyl-4-prop-2-enoyl-piperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[[1-[2-[4-[(3S)-4-tert-butoxycarbonyl-3-(cyanomethyl)piperazin-1-yl]-6-chloro-8-fluoro-7-(5-hydroxy-2,3-dimethyl-phenyl)quinazolin-2-yl]oxyethyl]-4-piperidyl]methoxy]acetic acid.

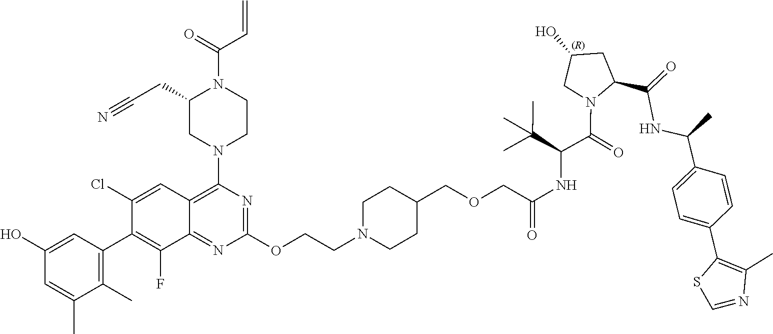

(formic acid salt, white solid). LC/MS (ESI) m/z: 1122.1 [M+H]⁺. ¹H-NMR (400 MHz, CD₃OD) δ 8.93-8.84 (m, 1H), 8.53 (s, 1H), 8.02 (s, 1H), 7.49-7.35 (m, 4H), 6.99-6.67 (m, 2H), 6.45 (d, J=2.4 Hz, 1H), 6.32 (d, J=16.8 Hz, 1H), 5.86 (d, J=10.0 Hz, 1H), 5.01 (q, J=6.8 Hz, 1H), 4.78-4.74 (m, 2H), 4.71 (s, 1H), 4.60-4.36 (m, 5H), 4.20-3.93 (m, 3H), 3.89-3.72 (m, 4H), 3.65-3.45 (m, 5H), 3.10-2.95 (m, 2H), 2.80-2.58 (m, 2H), 2.54-2.46 (m, 3H), 2.44-2.16 (m, 5H), 2.09-1.80 (m, 8H), 1.60-1.46 (m, 5H), 1.09-0.99 (m, 9H).

### Exemplary Synthesis of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(8-methyl-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 1-bromo-8-methyl-naphthalene

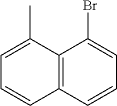

To a solution of 1,8-dibromonaphthalene (21.2 g, 74.0 mmol, 1 eq) in THF (400 mL) at 0° C. was added MeLi (1.6 M, 50.9 mL, 1.1 eq) dropwise, and the reaction mixture was stirred for 0.5 hour. CH₃I (42.0 g, 296.1 mmol, 4 eq) was then added under N₂, and the reaction mixture was stirred at 25° C. for 3 hours. The reaction was quenched by addition of water (80 mL) at 0° C., and the resulting mixture was extracted with EtOAc (2×300 mL). The combined organic extract was washed with brine (80 mL), dried over anhydrous sodium sulfate, filtered, and concentrated. The resulting residue was purified by prep-HPLC (55-95% CH₃CN in water (0.225% formic acid)) to afford 1-bromo-8-methyl-naphthalene (10.5 g, 45.1 mmol, 61% yield) as a yellow solid. ¹H-NMR (400 MHz, CDCl₃) δ 7.85 (d, J=7.6 Hz, 1H), 7.80 (d, J=8.0 Hz, 1H), 7.76-7.70 (m, 1H), 7.40-7.35 (m, 2H), 7.23 (t, J=8.0 Hz, 1H), 3.15 (s, 3H).

### Step 2: Preparation of 4,4,5,5-tetramethyl-2-(8-methyl-1-naphthyl)-1,3,2-dioxaborolane

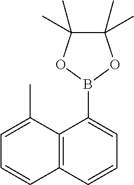 [SMILES: Cc1cccc2cccc(B3OC(C)(C)C(C)(C)O3)c12]

To 1-bromo-8-methyl-naphthalene (2.00 g, 9.05 mmol, 1 eq) in DMF (30 mL) were added KOAc (2.66 g, 27.1 mmol, 3 eq), 4,4,5,5-tetramethyl-2-(4,4,5,5-tetramethyl-1,3,2-dioxaborolan-2-yl)-1,3,2-dioxaborolane (4.59 g, 18.1 mmol, 2 eq), and Pd(dppf)C12 (662 mg, 0.905 mmol, 0.1 eq) under N₂ atmosphere, and the reaction mixture was stirred at 90° C. for 15 hours. The reaction mixture was cooled and diluted with EtOAc (100 mL). The resulting mixture was washed with water (3×20 mL), brine (3×20 mL), dried over Na₂SO₄, filtered, and concentrated. The resulting residue was purified by flash silica gel chromatography (gradient: 0-4% EtOAc in petroleum ether) followed by prep-HPLC (25-90% CH₃CN in water (10 mM NH₄HCO₃)) to give 4,4,5,5-tetramethyl-2-(8-methyl-1-naphthyl)-1,3,2-dioxaborolane (1.35 g, 5.01 mmol, 55% yield) as a yellow solid. LC/MS (ESI) m/z: 269.4 [M+H]⁺.

### Step 3: Preparation of tert-butyl(2S)-4-[6-chloro-2-(2,2-dimethoxyethoxy)-8-fluoro-7-(8-methyl-1-naphthyl)quinazolin-4-yl]-2-(cyanomethyl)piperazine-1-carboxylate

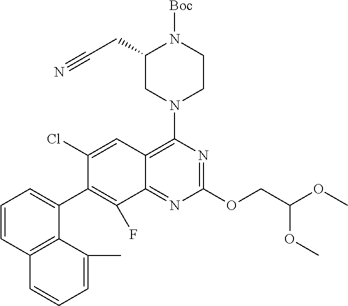 [SMILES: COC(COc1nc(N2CCN(C(=O)OC(C)(C)C)[C@@H](CC#N)C2)c2cc(Cl)c(-c3cccc4cccc(C)c34)c(F)c2n1)OC]

To a solution of tert-butyl (2S)-4-[7-bromo-6-chloro-2-(2,2-dimethoxyethoxy)-8-fluoro-quinazolin-4-yl]-2-(cyanomethyl)piperazine-1-carboxylate (880 mg, 1.49 mmol, 1 eq) and 4,4,5,5-tetramethyl-2-(8-methyl-1-naphthyl)-1,3,2-dioxaborolane (601 mg, 2.24 mmol, 1.5 eq) in dioxane (14 mL) were added aqueous K₃PO₄ (1.5 M, 3.49 mL, 3.5 eq) and XPhos Pd G3 (126.5 mg, 0.149 mmol, 0.1 eq), and the reaction mixture was stirred at 100° C. for 16 hours under N₂. The reaction mixture was diluted with EtOAc (120 mL), and the organic phase was washed with brine (30 mL), dried over anhydrous sodium sulfate, filtered, and concentrated. The resulting residue was purified by flash chromatography on SiO₂ (gradient: 10-25% EtOAc in petroleum ether) followed by prep-HPLC (50-80% CH₃CN in water (0.225% formic acid)). Pure fractions were combined and adjusted to pH=8 with solid NaHCO₃ and concentrated in vacuo to remove most CH₃CN. The resulting mixture was extracted with EtOAc (3×100 mL), and the resulting organic extract was washed with brine (40 mL), dried over anhydrous sodium sulfate, filtered, and concentrated to give tert-butyl(2S)-4-[6-chloro-2-(2,2-dimethoxyethoxy)-8-fluoro-7-(8-methyl-1-naphthyl)quinazolin-4-yl]-2-(cyanomethyl)piperazine-1-carboxylate (450 mg, 45% yield) as a yellow solid. LC/MS (ESI) m/z: 650.3 [M+H]⁺.

### Step 4: Preparation of (2S,4R)-1-[(2S)-2-[[2-[[1-[2-[6-chloro-4-[(3S)-3-(cyanomethyl)-4-prop-2-enoyl-piperazin-1-yl]-8-fluoro-7-(8-methyl-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[2-[6-Chloro-4-[(3S)-3-(cyanomethyl)-4-prop-2-enoyl-piperazin-1-yl]-8-fluoro-7-(8-methyl-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from tert-butyl(2S)-4-[6-chloro-2-(2,2-dimethoxyethoxy)-8-fluoro-7-(8-methyl-1-naphthyl)quinazolin-4-yl]-2-(cyanomethyl)piperazine-1-carboxylate.

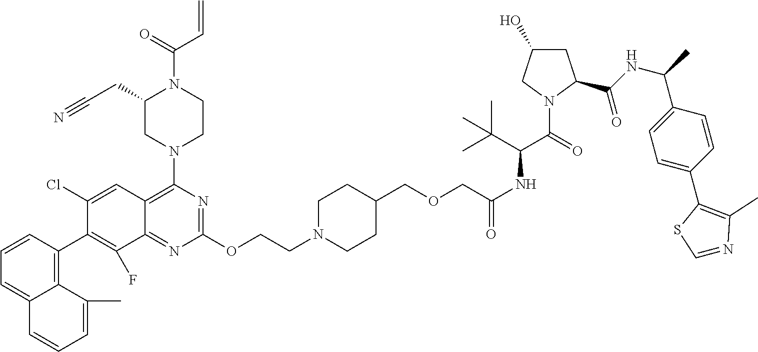 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cccc5cccc(C)c45)c(Cl)cc23)C[C@@H]1CC#N]

(formic acid salt, off-white solid). LC/MS (ESI) m/z: 1141.7 [M+H]⁺. ¹H-NMR (400 MHz, CD₃OD) δ 8.87 (s, 1H), 8.53 (s, 1H), 8.11-7.93 (m, 2H), 7.84 (d, J=7.6 Hz, 1H), 7.60-7.49 (m, 1H), 7.48-7.32 (m, 5H), 7.32-7.21 (m, 2H), 7.01-6.72 (m, 1H), 6.40-6.24 (m, 1H), 5.84 (d, J=8.4 Hz, 1H), 5.07-5.00 (m, 1H), 4.75-4.40 (m, 10H), 4.05-3.67 (m, 6H), 3.62-3.31 (m, 4H), 3.25-2.96 (m, 4H), 2.73-2.51 (m, 2H), 2.46 (s, 3H), 2.27-2.16 (m, 1H), 2.07 (s, 3H), 2.00-1.77 (m, 4H), 1.62-1.47 (m, 2H), 1.46 (d, J=5.2 Hz, 3H), 1.02 (s, 9H).

### Exemplary Synthesis of (2S,4R)-1-[(2S)-2-[[2-[[1-[(2R)-2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 2-[(2S)-4-[6-chloro-8-fluoro-7-(3-hydroxy-1-naphthyl)-2-[(1R)-1-methyl-2-oxo-ethoxy]quinazolin-4-yl]piperazin-2-yl]acetonitrile

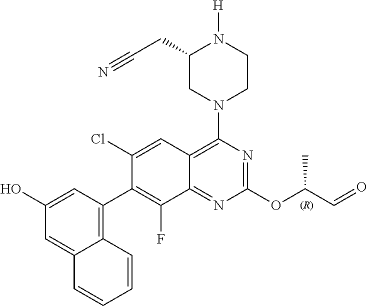

To a solution of tert-butyl (2S)-4-[6-chloro-2-[(1R)-2,2-dimethoxy-1-methyl-ethoxy]-8-fluoro-7-(3-hydroxy-1-naphthyl)quinazolin-4-yl]-2-(cyanomethyl)piperazine-1-carboxylate (800 mg, 1.20 mmol, 1 eq) in acetone (1.82 mL) at 0° C. was added conc. HCl (1.82 mL), and the reaction mixture was stirred at 20° C. for 0.5 hour. The mixture was diluted with water (5 mL), adjusted to pH=−7 with saturated aqueous NaHCO₃, and then filtered. The filter cake was diluted with THF (60 mL), and the resulting mixture was dried over Na₂SO₄, filtered, and concentrated to give 2-[(2S)-4-[6-chloro-8-fluoro-7-(3-hydroxy-1-naphthyl)-2-[(1R)-1-methyl-2-oxo-ethoxy]quinazolin-4-yl]piperazin-2-yl]acetonitrile (630 mg, crude) as a yellow solid. LC/MS (ESI) m/z: 520.1 [M+H]⁺.

### Step 2: Preparation of (2S,4R)-1-[(2S)-2-[[2-[[1-[(2R)-2-[6-chloro-4-[(3S)-3-(cyanomethyl)- 4-prop-2-enoyl-piperazin-1-yl]-8-fluoro-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[[1-[(2R)-2-[6-Chloro-4-[(3S)-3-(cyanomethyl)-4-prop-2-enoyl- piperazin-1-yl]-8-fluoro-7-(3-hydroxy-1-naphthyl)quinazolin-2-yl]oxypropyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-4-[(3S)-3-(cyanomethyl)-4-prop-2-enoyl- piperazin-1-yl]-8-fluoro-7-(3-hydroxy-1-naphthyl)quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[(2S)-4-[6-chloro-8-fluoro-7-(3-hydroxy-1-naphthyl)-2-[(1R)-1-methyl-2-oxo-ethoxy]quinazolin-4-yl]piperazin-2-yl]acetonitrile.

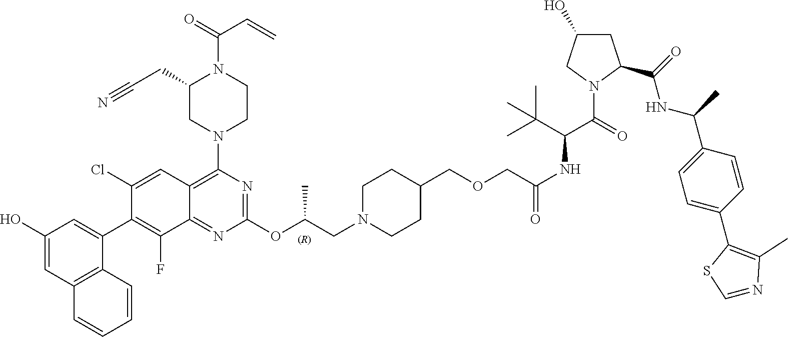

(formic acid salt, white solid). LC/MS (ESI) m/z: 1157.7 [M+H]⁺. ¹H-NMR (400 MHz, CD₃OD) δ 8.92-8.83 (m, 1H), 8.07 (s, 1H), 7.74 (d, J=8.4 Hz, 1H), 7.48-7.33 (m, 4H), 7.28-7.15 (m, 3H), 7.03 (s, 1H), 6.98-6.74 (m, 1H), 6.31 (d, J=17.2 Hz, 1H), 5.90-5.79 (m, 1H), 5.70-5.52 (m, 1H), 5.17-4.94 (m, 1H), 4.85-4.80 (m, 1H), 4.69-4.52 (m, 5H), 4.50-4.40 (m, 3H), 4.22-4.09 (m, 1H), 3.96-3.80 (m, 4H), 3.77-3.63 (m, 2H), 3.59-3.47 (m, 1H), 3.17-2.96 (m, 4H), 2.85-2.70 (m, 1H), 2.59-2.51 (m, 1H), 2.47 (s, 3H), 2.26-2.15 (m, 2H), 2.14-2.07 (m, 1H), 2.01-1.90 (m, 1H), 1.77-1.59 (m, 3H), 1.57-1.44 (m, 3H), 1.39 (d, J=6.0 Hz, 3H), 1.27-1.12 (m, 2H), 1.01 (s, 9H).

### Exemplary Synthesis of (2S,4R)-1-[(2S)-2-[[2-[3-[[4-[6-chloro-4-[(3S)-3-(cyanomethyl)-4-prop-2-enoyl-piperazin-1-yl]-8-fluoro-7-(3-hydroxy-1-naphthyl)quinazolin-2-yl]piperazin-1-yl]methyl]cyclobutoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of 3-(benzyloxymethyl)cyclobutanol

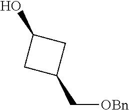 [SMILES: O[C@H]1C[C@@H](COCc2ccccc2)C1]

To a solution of 3-(benzyloxymethyl)cyclobutanone (25.0 g, 131 mmol, 1 eq) in THF (100 mL) at −70° C. was added L-selectride (151 mL, 151 mmol, 1.15 eq, 1 M in THF) dropwise, and the reaction mixture was stirred at 20° C. for 16 hours. The reaction was quenched with saturated aqueous NH₄Cl (100 mL), and the resulting mixture was extracted with EtOAc (3×150 mL). The combined organic extract was washed with brine (300 mL), dried over anhydrous Na₂SO₄, and concentrated. The resulting crude product was purified by flash silica gel chromatography (gradient: 0-20% THF in petroleum ether) to give 3-(benzyloxymethyl)cyclobutanol (23.2 g, 121 mmol, 92% yield) as a colorless oil. LC/MS (ESI) m/z: 193.2 [M+H]⁺.

### Step 2: Preparation of [3-(benzyloxymethyl)cyclobutyl]4-nitrobenzoate

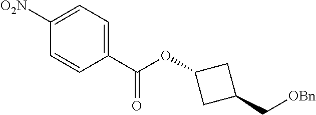 [SMILES: O=C(O[C@H]1C[C@H](COCc2ccccc2)C1)c1ccc([N+](=O)[O-])cc1]

To a solution of 3-(benzyloxymethyl)cyclobutanol (10.0 g, 52.0 mmol, 1 eq), 4-nitrobenzoic acid (10.4 g, 62.4 mmol, 1.2 eq), and PPh₃ (17.7 g, 67.6 mmol, 1.3 eq) in THF (200 mL) at 0° C. was added DIAD (13.7 g, 67.6 mmol, 13.2 mL, 1.3 eq) under N₂, and the reaction mixture was stirred at 20° C. for 6 hours. The reaction mixture was concentrated under reduced pressure, and the resulting crude product was purified by flash silica gel chromatography (gradient: 0-10% EtOAc in petroleum ether) to give [3-(benzyloxymethyl)cyclobutyl]4-nitrobenzoate (16.1 g, 47.2 mmol, 91% yield) as a white solid. LC/MS (ESI) m/z: 342.0 [M+H]⁺.

### Step 3: Preparation of 3-(benzyloxymethyl)cyclobutanol

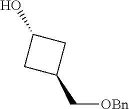 [SMILES: O[C@H]1C[C@H](COCc2ccccc2)C1]

To a solution of [3-(benzyloxymethyl)cyclobutyl]4-nitrobenzoate (16.1 g, 47.2 mmol, 1 eq) in THF (80 mL) was added a solution of NaOH (3.77 g, 94.3 mmol, 2 eq) in H₂O (80 mL), and the reaction mixture was stirred at 20° C. for 16 hours. The mixture was extracted with EtOAc (3×100 mL), and the combined organic extract was washed with brine (2×80 mL), dried over anhydrous Na₂SO₄, and concentrated. The resulting crude product was purified by flash silica gel chromatography (gradient: 0-15% THF in petroleum ether) to give 3-(benzyloxymethyl)cyclobutanol (8.55 g, 44.5 mmol, 94% yield) as a colorless oil. LC/MS (ESI) m/z: 193.2 [M+H]⁺.

### Step 4: Preparation of tert-butyl 2-[3-(benzyloxymethyl)cyclobutoxy]acetate

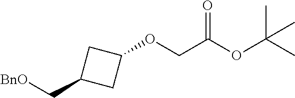 [SMILES: CC(C)(C)OC(=O)CO[C@H]1C[C@H](COCc2ccccc2)C1]

To a solution of 3-(benzyloxymethyl)cyclobutanol (300 mg, 1.56 mmol, 1 eq) in DMF (3 mL) at 0° C. was added NaH (100 mg, 2.50 mmol, 60% purity, 1.6 eq), and the reaction mixture was stirred at 20° C. under N₂ for 15 minutes. tert-Butyl 2-bromoacetate (396 mg, 2.03 mmol, 1.3 eq) was then added, and the reaction mixture was stirred at 20° C. under N₂ for 16 hours. The reaction mixture was quenched by addition of water (15 mL) and then extracted with EtOAc (3×15 mL). The combined organic extract was washed with brine (3×15 mL), dried over anhydrous Na₂SO₄, filtered, and concentrated. The resulting crude product was purified by prep-TLC (acidic silica gel, petroleum ether/ethyl acetate=3:1) to give tert-butyl 2-[3-(benzyloxymethyl)cyclobutoxy]acetate (253 mg, 0.826 mmol, 53% yield) as a colorless oil. LC/MS (ESI) m/z: 251.2 [M+H]⁺.

### Step 5: Preparation of tert-butyl 2-[3-(hydroxymethyl)cyclobutoxy]acetate

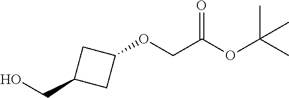

To a solution of tert-butyl 2-[3-(benzyloxymethyl)cyclobutoxy]acetate (1.19 g, 3.88 mmol, 1 eq) in EtOH (30 mL) was added Pd/C (120 mg, 0.777 mmol, 10% purity, 0.2 eq), and the reaction mixture was stirred at 65° C. under H₂ (50 psi) for 24 hours. The reaction mixture was filtered and the filtrate was concentrated under reduced pressure to give tert-butyl 2-[3-(hydroxymethyl)cyclobutoxy]acetate (829 mg, 3.83 mmol, 99% yield) as a colorless oil.

### Step 6: Preparation of tert-butyl 2-[3-(p-tolylsulfonyloxymethyl)cyclobutoxy]acetate

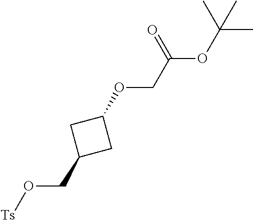 [SMILES: Cc1ccc(S(=O)(=O)OC[C@H]2C[C@H](OCC(=O)OC(C)(C)C)C2)cc1]

To a solution of tert-butyl 2-[3-(hydroxymethyl)cyclobutoxy]acetate (829 mg, 3.83 mmol, 1 eq), TEA (1.16 g, 11.5 mmol, 1.60 mL, 3 eq), and DMAP (234 mg, 1.92 mmol, 0.5 eq) in DCM (20 mL) at 0° C. was added TosCl (1.46 g, 7.67 mmol, 2 eq), and the reaction mixture was stirred at 20° C. under N₂ for 16 hours. The reaction mixture was poured onto water (20 mL), and the resulting aqueous mixture was extracted with CH₂Cl₂ (2×20 mL). The combined organic extract was washed with brine (20 mL), dried over anhydrous Na₂SO₄, filtered, and concentrated. The resulting crude product was purified by flash silica gel chromatography (gradient: 0-15% THF in petroleum ether) to give tert-butyl 2-[3-(p-tolylsulfonyloxymethyl)cyclobutoxy]acetate (1.33 g, 3.59 mmol, 94% yield) as a colorless oil. ¹H-NMR (400 MHz, CDCl₃) δ 7.80 (d, J=8.4 Hz, 2H), 7.36 (d, J=8.0 Hz, 2H), 4.13-4.04 (m, 1H), 4.02 (d, J=6.8 Hz, 2H), 3.84 (s, 2H), 2.61-2.50 (m, 1H), 2.46 (s, 3H), 2.24-2.14 (m, 2H), 2.11-2.01 (m, 2H), 1.47 (s, 9H).

### Step 7: Preparation of benzyl 4-[[3-(2-tert-butoxy-2-oxo-ethoxy)cyclobutyl]methyl]piperazine-1-carboxylate

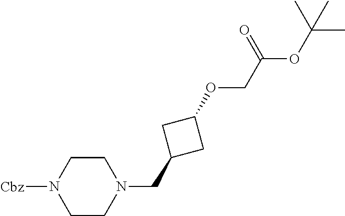 [SMILES: CC(C)(C)OC(=O)CO[C@H]1C[C@H](CN2CCN(C(=O)OCc3ccccc3)CC2)C1]

To a solution of benzyl piperazine-1-carboxylate (856 mg, 3.89 mmol, 1.2 eq) in CH₃CN (20 mL) were added diisopropylethylamine (628 mg, 4.86 mmol, 1.5 eq), KI (108 mg, 0.648 mmol, 0.2 eq), and tert-butyl 2-[3-(p-tolylsulfonyloxymethyl)cyclobutoxy]acetate (1.20 g, 3.24 mmol, 1 eq), and the reaction mixture was stirred at 80° C. under N₂ for 24 hours. The reaction mixture was poured onto water (30 mL), and the resulting aqueous mixture was extracted with EtOAc (3×30 mL). The combined organic extract was washed with brine (2×30 mL), dried over anhydrous Na₂SO₄, filtered, and concentrated. The resulting crude product was purified by flash silica gel chromatography (gradient: 0˜-5% THF in petroleum ether) to give benzyl 4-[[3-(2-tert-butoxy-2-oxo-ethoxy)cyclobutyl]methyl]piperazine-1-carboxylate (1.07 g, 2.56 mmol, 79% yield) as a colorless oil. LC/MS (ESI) m/z: 419.3 [M+H]⁺.

### Step 8: Preparation of tert-butyl 2-[3-(piperazin-1-ylmethyl)cyclobutoxy]acetate

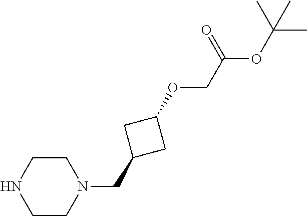 [SMILES: CC(C)(C)OC(=O)CO[C@H]1C[C@H](CN2CCNCC2)C1]

To a solution of benzyl 4-[[3-(2-tert-butoxy-2-oxo-ethoxy)cyclobutyl]methyl]piperazine-1-carboxylate (1.16 g, 2.77 mmol, 1 eq) in isopropanol (20 mL) was added Pd/C (120 mg, 0.554 mmol, 10% purity, 0.2 eq), and the reaction mixture was stirred at 35° C. under H₂ (15 psi) for 24 hours. The reaction mixture was filtered, and the filtrate was concentrated under reduced pressure to give tert-butyl 2-[3-(piperazin-1-ylmethyl)cyclobutoxy]acetate (820 mg, crude) as a colorless oil. ¹H-NMR (400 MHz, CDCl₃) δ 4.19-4.10 (m, 1H), 3.87 (s, 2H), 3.00-2.93 (m, 3H), 2.53-2.45 (m, 4H), 2.45-2.42 (m, 2H), 2.24-2.14 (m, 3H), 2.07-1.97 (m, 3H), 1.48 (s, 9H).

### Step 9: Preparation of tert-butyl (2S)-4-[7-bromo-2-[4-[[3-(2-tert-butoxy-2-oxo-ethoxy)cyclobutyl]methyl]piperazin-1-yl]-6-chloro-8-fluoro-quinazolin-4-yl]-2-(cyanomethyl)piperazine-1-carboxylate

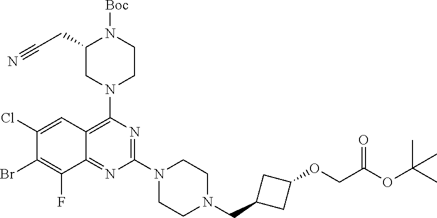 [SMILES: CC(C)(C)OC(=O)CO[C@H]1C[C@H](CN2CCN(c3nc(N4CCN(C(=O)OC(C)(C)C)[C@@H](CC#N)C4)c4cc(Cl)c(Br)c(F)c4n3)CC2)C1]

To a solution of tert-butyl 2-[3-(piperazin-1-ylmethyl)cyclobutoxy]acetate (300 mg, 1.05 mmol, 1 eq) and tert-butyl (2S)-4-(7-bromo-2,6-dichloro-8-fluoro-quinazolin-4-yl)-2-(cyanomethyl)piperazine-1-carboxylate (545 mg, 1.05 mmol, 1 eq) in THF (10 mL) were added diisopropylethylamine (204 mg, 1.58 mmol, 1.5 eq), and the reaction mixture was stirred at 50° C. under N₂ for 2 hours. Additional tert-butyl 2-[3-(piperazin-1-ylmethyl)cyclobutoxy]acetate (100 mg, 0.352 mmol, 0.33 eq) was added, and the reaction mixture was stirred at 50° C. under N₂ for 12 hours. The reaction mixture was concentrated under reduced pressure, and the resulting crude product was purified by flash silica gel chromatography (gradient: 0-70% EtOAc in petroleum ether) to give tert-butyl (2S)-4-[7-bromo-2-[4-[[3-(2-tert-butoxy-2-oxo-ethoxy)cyclobutyl]methyl]piperazin-1-yl]-6-chloro-8-fluoro-quinazolin-4-yl]-2-(cyanomethyl)piperazine-1-carboxylate (610 mg, 0.795 mmol, 76% yield) as a yellow oil. LC/MS (ESI) m/z: 768.4 [M+H]⁺.

### Step 10: Preparation of tert-butyl (2S)-4-[2-[4-[[3-(2-tert-butoxy-2-oxo-ethoxy)cyclobutyl]methyl]piperazin-1-yl]-6-chloro-8-fluoro-7-(3-hydroxy-1-naphthyl)quinazolin-4-yl]-2-(cyanomethyl)piperazine-1-carboxylate

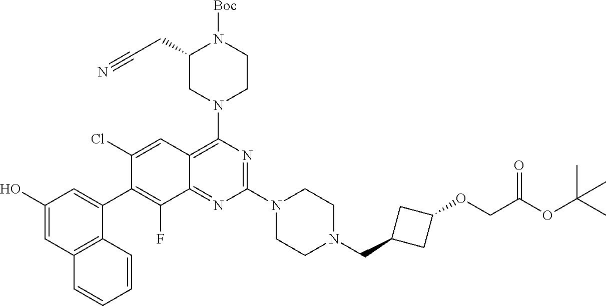 [SMILES: CC(C)(C)OC(=O)CO[C@H]1C[C@H](CN2CCN(c3nc(N4CCN(C(=O)OC(C)(C)C)[C@@H](CC#N)C4)c4cc(Cl)c(-c5cc(O)cc6ccccc56)c(F)c4n3)CC2)C1]

To a solution of tert-butyl (2S)-4-[7-bromo-2-[4-[[3-(2-tert-butoxy-2-oxo-ethoxy)cyclobutyl]methyl]piperazin-1-yl]-6-chloro-8-fluoro-quinazolin-4-yl]-2-(cyanomethyl)piperazine-1-carboxylate (610 mg, 0.795 mmol, 1 eq) in THF (5 mL) were added 4-(4,4,5,5-tetramethyl-1,3,2-dioxaborolan-2-yl)naphthalen-2-ol (215 mg, 0.795 mmol, 1 eq), aqueous K₃PO₄ (1.5 M, 1.59 mL, 2.39 mmol, 3 eq), and XPhos Pd G3 (34 mg, 0.040 mmol, 0.05 eq), and the reaction mixture was stirred at 50° C. under N₂ for 2 hours. The reaction mixture was diluted with EtOAc (50 mL), and the resulting mixture was dried over anhydrous Na₂SO₄ and filtered. The filtrate was concentrated under reduced pressure, and the resulting crude product was purified by flash silica gel chromatography (gradient: 0-60% THF in petroleum) to give tert-butyl (2S)-4-[2-[4-[[3-(2-tert-butoxy-2-oxo-ethoxy)cyclobutyl]methyl]piperazin-1-yl]-6-chloro-8-fluoro-7-(3-hydroxy-1-naphthyl)quinazolin-4-yl]-2-(cyanomethyl)piperazine-1-carboxylate (423 mg, 0.509 mmol, 64% yield) as a yellow solid.

### Step 11: Preparation of 2-[3-[[4-[4-[(3S)-4-tert-butoxycarbonyl-3-(cyanomethyl)piperazin-1-yl]-6-chloro-8-fluoro-7-(3-hydroxy-1-naphthyl)quinazolin-2-yl]piperazin-1-yl]methyl]cyclobutoxy]acetic Acid

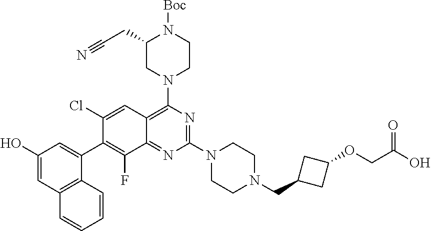 [SMILES: CC(C)(C)OC(=O)N1CCN(c2nc(N3CCN(C[C@H]4C[C@H](OCC(=O)O)C4)CC3)nc3c(F)c(-c4cc(O)cc5ccccc45)c(Cl)cc23)C[C@@H]1CC#N]

To a solution of tert-butyl (2S)-4-[2-[4-[[3-(2-tert-butoxy-2-oxo-ethoxy)cyclobutyl]methyl]piperazin-1-yl]-6-chloro-8-fluoro-7-(3-hydroxy-1-naphthyl)quinazolin-4-yl]-2-(cyanomethyl)piperazine-1-carboxylate (220 mg, 0.265 mmol, 1 eq) in THF (1 mL), water (1 mL), and CH₃OH (1 mL) was added LiOH.H₂O (334 mg, 7.95 mmol, 30 eq), and the reaction mixture was stirred at 20° C. for 0.5 hour. The reaction mixture was diluted with water (10 mL), and the resulting aqueous mixture was adjusted to pH=5 with 2 N aqueous HCl. The mixture was extracted with 3:1 EtOAc/THF (3×20 mL), and the combined organic extract was washed with brine (2×15 mL), dried over anhydrous Na₂SO₄, and concentrated under reduced pressure to give 2-[3-[[4-[4-[(3S)-4-tert-butoxycarbonyl-3-(cyanomethyl)piperazin-1-yl]-6-chloro-8-fluoro-7-(3-hydroxy-1-naphthyl)quinazolin-2-yl]piperazin-1-yl]methyl]cyclobutoxy]acetic acid (204 mg, 0.263 mmol, 99% yield) as a yellow solid. LC/MS (ESI) m/z: 774.6 [M+H]⁺.

### Step 12: Preparation of (2S,4R)-1-[(2S)-2-[[2-[3-[[4-[6-chloro-4-[(3S)-3-(cyanomethyl)-4-prop-2-enoyl-piperazin-1-yl]-8-fluoro-7-(3-hydroxy-1-naphthyl)quinazolin-2-yl]piperazin-1-yl]methyl]cyclobutoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[3-[[4-[6-Chloro-4-[(3S)-3-(cyanomethyl)-4-prop-2-enoyl-piperazin-1-yl]-8-fluoro-7-(3-hydroxy-1-naphthyl)quinazolin-2-yl]piperazin-1-yl]methyl]cyclobutoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-8-fluoro-7-(3-hydroxy-1-naphthyl)-4-[(2S)-2-methyl-4-prop-2-enoyl-piperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[3-[[4-[4-[(3S)-4-tert-butoxycarbonyl-3-(cyanomethyl)piperazin-1-yl]-6-chloro-8-fluoro-7-(3-hydroxy-1-naphthyl)quinazolin-2-yl]piperazin-1-yl]methyl]cyclobutoxy]acetic acid.

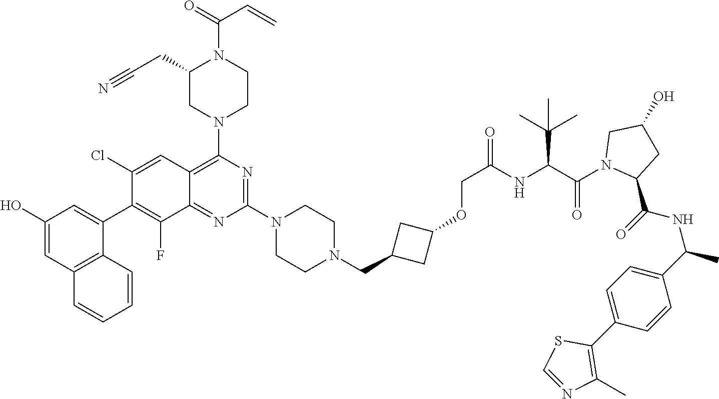 [SMILES: C=CC(=O)N1CCN(c2nc(N3CCN(C[C@H]4C[C@H](OCC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)C[C@@H]1CC#N]

(formic acid salt, white solid). LC/MS (ESI) m/z: 1154.5 [M+H]⁺. ¹H-NMR (400 MHz, CD₃OD) δ 8.91-8.78 (m, 1H), 8.42 (s, 1H), 7.88 (s, 1H), 7.74 (d, J=8.4 Hz, 1H), 7.48-7.31 (m, 5H), 7.29-7.10 (m, 3H), 7.04-6.96 (m, 1H), 6.94-6.70 (m, 1H), 6.30 (d, J=16.8 Hz, 1H), 5.84 (d, J=11.2 Hz, 1H), 5.03-4.96 (m, 3H), 4.63-4.51 (m, 2H), 4.47-4.28 (m, 2H), 4.24-3.69 (m, 10H), 3.68-3.38 (m, 4H), 3.08-2.60 (m, 8H), 2.50-2.39 (m, 3H), 2.36-2.06 (m, 5H), 2.03-1.88 (m, 1H), 1.59-1.43 (m, 3H), 1.10-0.97 (m, 9H).

### Exemplary Synthesis of (2S,4R)-1-[(2S)-2-[[2-[4-[(2S)-2-[[6-chloro-4-[(3S)-3-(cyanomethyl)-4-prop-2-enoyl-piperazin-1-yl]-8-fluoro-7-(3-hydroxy-1-naphthyl)quinazolin-2-yl]oxymethyl]pyrrolidin-1-yl]butoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 2-(4-hydroxybutoxy)acetate

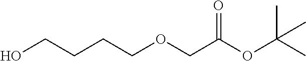

To a solution of butane-1,4-diol (13.86 g, 153.80 mmol, 3 eq) in CH₂Cl₂ (128 mL) were added tetrabutylammonium bromide (16.53 g, 51.27 mmol, 1 eq) and aqueous NaOH (12.08 M, 96.13 mL, 35% wt., 22.65 eq) followed by tert-butyl 2-bromoacetate (10.0 g, 51.27 mmol, 7.58 mL, 1 eq) dropwise, and the reaction mixture was stirred at 20° C. for 16 hours. The mixture was filtered, and the filter cake was washed with CH₂Cl₂ (150 mL). The organic phase was separated, and the aqueous phase was further extracted with CH₂Cl₂ (3×100 mL). The combined organic extract was washed with water (100 mL) and brine (80 mL), dried over anhydrous sodium sulfate, filtered, and concentrated. The resulting residue was purified by flash chromatography on SiO₂ (gradient: 10-30% EtOAc in petroleum ether) to afford tert-butyl 2-(4-hydroxybutoxy)acetate (2.07 g, 10.13 mmol, 20% yield) as a colorless oil. ¹H-NMR (400 MHz, CDCl₃) δ 3.97 (s, 2H), 3.69 (t, J=6.0 Hz, 2H), 3.57 (t, J=6.0 Hz, 2H), 1.99 (br s, 1H), 1.80-1.63 (m, 4H), 1.49 (s, 9H).

### Step 2: Preparation of tert-butyl2-[4-(p-tolylsulfonyloxy)butoxy]acetate

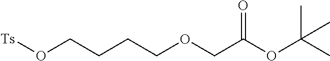 [SMILES: Cc1ccc(S(=O)(=O)OCCCCOCC(=O)OC(C)(C)C)cc1]

To a solution of tert-butyl 2-(4-hydroxybutoxy)acetate (300 mg, 1.47 mmol, 1 eq), Et₃N (520.2 mg, 5.14 mmol, 3.5 eq), and DMAP (9.0 mg, 0.073 mmol, 0.05 eq) in CH₂Cl₂ (12 mL) was added TosCl (308.0 mg, 1.62 mmol, 1.1 eq), and the reaction mixture was stirred at 20° C. for 16 hours. Water (15 mL) was then added, and the resulting mixture was extracted with CH₂Cl₂ (3×20 mL). The combined organic extract was washed with brine (30 mL), dried over anhydrous sodium sulfate, filtered, and concentrated. The resulting residue was purified by flash chromatography on SiO₂ (gradient: 0-15% EtOAc in petroleum ether) to afford tert-butyl 2-[4-(p-tolylsulfonyloxy)butoxy]acetate (280 mg, 0.781 mmol, 53% yield) as a colorless oil. ¹H-NMR (400 MHz, CDCl₃) δ 7.80 (d, J=8.4 Hz, 2H), 7.35 (d, J=8.0 Hz, 2H), 4.08 (t, J=6.4 Hz, 2H), 3.90 (s, 2H), 3.48 (t, J=6.0 Hz, 2H), 2.46 (s, 3H), 1.85-1.73 (m, 2H), 1.70-1.60 (m, 2H), 1.47 (s, 9H).

### Step 3: Preparation of 2-trimethylsilylethyl (2S)-2-(hydroxymethyl)pyrrolidine-1-carboxylate

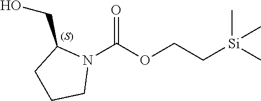

To a solution of [(2S)-pyrrolidin-2-yl]methanol (2 g, 19.77 mmol, 1.92 mL, 1 eq) and Na₂CO₃ (2.51 g, 23.73 mmol, 1.2 eq) in THF (15 mL) and H₂O (15 mL) was added (2,5-dioxopyrrolidin-1-yl) 2-trimethylsilylethyl carbonate (3.08 g, 11.86 mmol, 0.6 eq) portionwise, and the reaction mixture was stirred at 20° C. for 16 hours. The reaction mixture was partitioned between water (15 mL) and EtOAc (30 mL). The organic phase was separated, and the aqueous phase was further extracted with EtOAc (3×30 mL). The combined organic extract was washed with brine (40 mL), dried over anhydrous sodium sulfate, filtered, and concentrated. The resulting residue was purified by flash chromatography on SiO₂ (gradient: 0-15% EtOAc in petroleum ether) to afford 2-trimethylsilylethyl (2S)-2-(hydroxymethyl)pyrrolidine-1-carboxylate (2.13 g, 8.25 mmol, 42% yield) as a colorless oil. ¹H-NMR (400 MHz, CDCl₃) δ 4.20 (dd, J=9.2, 7.6 Hz, 2H), 4.06-3.92 (m, 1H), 3.73-3.58 (m, 2H), 3.56-3.46 (m, 1H), 3.40-3.30 (m, 1H), 2.09-1.97 (m, 1H), 1.93-1.73 (m, 2H), 1.58 (s, 1H), 1.09-0.98 (m, 2H), 0.05 (s, 9H).

### Step 4: Preparation of tert-butyl (2S)-4-[7-bromo-6-chloro-8-fluoro-2-[[(2S)-1-(2-trimethylsilylethoxycarbonyl)pyrrolidin-2-yl]methoxy]quinazolin-4-yl]-2-(cyanomethyl)piperazine-1-carboxylate

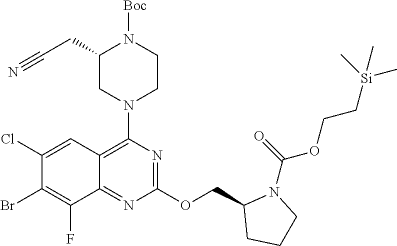 [SMILES: CC(C)(C)OC(=O)N1CCN(c2nc(OC[C@@H]3CCCN3C(=O)OCC[Si](C)(C)C)nc3c(F)c(Br)c(Cl)cc23)C[C@@H]1CC#N]

To a solution of tert-butyl (2S)-4-(7-bromo-2,6-dichloro-8-fluoro-quinazolin-4-yl)-2-(cyanomethyl)piperazine-1-carboxylate (1.2 g, 2.31 mmol, 1 eq) in CH₃CN (20 mL) were added DABCO (25.9 mg, 0.231 mmol, 0.1 eq), Cs₂CO₃ (979.0 mg, 3.00 mmol, 1.3 eq), and 2-trimethylsilylethyl (2S)-2-(hydroxymethyl)pyrrolidine-1-carboxylate (850.7 mg, 3.47 mmol, 1.5 eq), and the reaction mixture was stirred at 40° C. for 2 hours. The reaction mixture was filtered, and the filtrate was concentrated. The resulting residue was purified by flash chromatography on SiO₂ (gradient: 5-25% EtOAc in petroleum ether) to afford tert-butyl (2S)-4-[7-bromo-6-chloro-8-fluoro-2-[[(2S)-1-(2-trimethylsilylethoxycarbonyl)pyrrolidin-2-yl]methoxy]quinazolin-4-yl]-2-(cyanomethyl)piperazine-1-carboxylate (1.55 g, 1.81 mmol, 78% yield) as an oily yellow solid. ¹H-NMR (400 MHz, CDCl₃) δ 7.77-7.69 (m, 1H), 4.76-4.62 (m, 2H), 4.33-4.15 (m, 6H), 4.05-3.94 (m, 1H), 3.85-3.57 (m, 2H), 3.52-3.29 (m, 4H), 2.97-2.68 (m, 2H), 2.12-2.07 (m, 1H), 2.03-1.96 (m, 1H), 1.93-1.76 (m, 2H), 1.51 (s, 9H), 1.08-0.95 (m, 2H), 0.05 (s, 9H).

### Step 5: Preparation of tert-butyl (2S)-4-[7-bromo-6-chloro-8-fluoro-2-[[(2S)-pyrrolidin-2-yl]methoxy]quinazolin-4-yl]-2-(cyanomethyl)piperazine-1-carboxylate

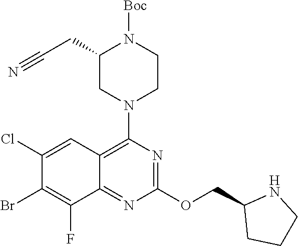 [SMILES: CC(C)(C)OC(=O)N1CCN(c2nc(OC[C@@H]3CCCN3)nc3c(F)c(Br)c(Cl)cc23)C[C@@H]1CC#N]

To a solution of tert-butyl (2S)-4-[7-bromo-6-chloro-8-fluoro-2-[[(2S)-1-(2-trimethylsilylethoxycarbonyl)pyrrolidin-2-yl]methoxy]quinazolin-4-yl]-2-(cyanomethyl)piperazine-1-carboxylate (1.4 g, 1.92 mmol, 1 eq) in DMF (20 mL) was added CsF (1.46 g, 9.61 mmol, 5 eq), and the reaction mixture was stirred at 60° C. for 2 hours. The reaction was diluted with EtOAc (120 mL) and then washed with water (4×30 mL), brine (2×30 mL), dried over anhydrous sodium sulfate, filtered, and concentrated. The resulting residue was purified by flash chromatography on SiO₂ (gradient: 10-32% EtOAc in petroleum ether to afford tert-butyl (2S)-4-[7-bromo-6-chloro-8-fluoro-2-[[(2S)-pyrrolidin-2-yl]methoxy]quinazolin-4-yl]-2-(cyanomethyl)piperazine-1-carboxylate (0.6 g, 51% yield) as a yellow solid. ¹H-NMR (400 MHz, CDCl₃) δ 7.56 (d, J=2.0 Hz, 1H), 4.78-4.62 (m, 1H), 4.37-4.19 (m, 2H), 4.17-4.02 (m, 2H), 3.98-3.88 (m, 1H), 3.86-3.78 (m, 1H), 3.77-3.68 (m, 1H), 3.66-3.57 (m, 1H), 3.55-3.45 (m, 1H), 3.41-3.18 (m, 2H), 2.91-2.71 (m, 2H), 2.29-2.14 (m, 1H), 2.03-1.93 (m, 1H), 1.93-1.83 (m, 1H), 1.78-1.65 (m, 1H), 1.52 (s, 9H), 1.27 (t, J=7.2 Hz, 1H).

### Step 6: Preparation of tert-butyl(2S)-4-[7-bromo-2-[[(2S)-1-[4-(2-tert-butoxy-2-oxo-ethoxy)butyl]pyrrolidin-2-yl]methoxy]-6-chloro-8-fluoro-quinazolin-4-yl]-2-(cyanomethyl)piperazine-1-carboxylate

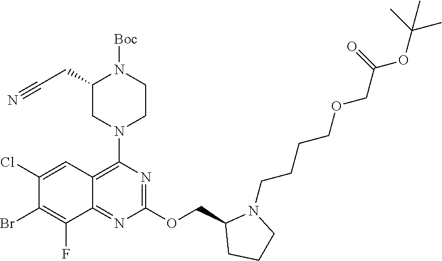 [SMILES: CC(C)(C)OC(=O)COCCCCN1CCC[C@H]1COc1nc(N2CCN(C(=O)OC(C)(C)C)[C@@H](CC#N)C2)c2cc(Cl)c(Br)c(F)c2n1]

To a solution of tert-butyl (2S)-4-[7-bromo-6-chloro-8-fluoro-2-[[(2S)-pyrrolidin-2-yl]methoxy]quinazolin-4-yl]-2-(cyanomethyl)piperazine-1-carboxylate (330.0 mg, 0.565 mmol, 1 eq) and tert-butyl 2-[4-(p-tolylsulfonyloxy)butoxy]acetate (607.8 mg, 1.70 mmol, 3.0 eq) in CH₃CN (10 mL) was added diisopropylethylamine (292.2 mg, 2.26 mmol, 4 eq), and the reaction mixture was stirred at 130° C. for 16 hours under microwave conditions. The mixture was concentrated, and the resulting residue was purified by flash chromatography on SiO₂ (gradient: 5-17% EtOAc in petroleum ether) to afford tert-butyl(2S)-4-[7-bromo-2-[[(2S)-1-[4-(2-tert-butoxy-2-oxo-ethoxy)butyl]pyrrolidin-2-yl]methoxy]-6-chloro-8-fluoro-quinazolin-4-yl]-2-(cyanomethyl)piperazine-1-carboxylate (230 mg, 0.299 mmol, 38% yield) as a yellow solid. LC/MS (ESI) m/z: 771.2 [M+H]⁺.

### Step 7: Preparation of tert-butyl (2S)-4-[2-[[(2S)-1-[4-(2-tert-butoxy-2-oxo-ethoxy)butyl]pyrrolidin-2-yl]methoxy]-6-chloro-8-fluoro-7-(3-hydroxy-1-naphthyl)quinazolin-4-yl]-2-(cyanomethyl)piperazine-1-carboxylate

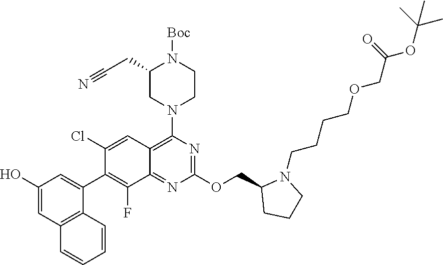 [SMILES: CC(C)(C)OC(=O)COCCCCN1CCC[C@H]1COc1nc(N2CCN(C(=O)OC(C)(C)C)[C@@H](CC#N)C2)c2cc(Cl)c(-c3cc(O)cc4ccccc34)c(F)c2n1]

To a solution of tert-butyl (2S)-4-[7-bromo-2-[[(2S)-1-[4-(2-tert-butoxy-2-oxo-ethoxy)butyl]pyrrolidin-2-yl]methoxy]-6-chloro-8-fluoro-quinazolin-4-yl]-2-(cyanomethyl)piperazine-1-carboxylate (230 mg, 0.299 mmol, 1 eq) and 4-(4,4,5,5-tetramethyl-1,3,2-dioxaborolan-2-yl)naphthalen-2-ol (145.2 mg, 0.538 mmol, 1.8 eq) in THF (4 mL) were added aqueous K₃PO₄ (1.5 M, 0.80 mL, 4.0 eq) and XPhos Pd G3 (12.6 mg, 0.015 mmol, 0.05 eq), and the reaction mixture was stirred at 50° C. for 16 hours under N₂. The reaction mixture was diluted with EtOAc (80 mL), and the organic phase was washed with brine (30 mL), dried over anhydrous sodium sulfate, filtered, and concentrated. The resulting the residue was purified by flash chromatography on SiO₂ (gradient: 5-30% EtOAc in petroleum ether to afford tert-butyl (2S)-4-[2-[[(2S)-1-[4-(2-tert-butoxy-2-oxo-ethoxy)butyl]pyrrolidin-2-yl]methoxy]-6-chloro-8-fluoro-7-(3-hydroxy-1-naphthyl)quinazolin-4-yl]-2-(cyanomethyl)piperazine-1-carboxylate (140 mg, 52% yield) as a yellow solid. LC/MS (ESI) m/z: 833.7 [M+H]⁺.

### Step 8: Preparation of 2-[4-[(2S)-2-[[4-[(3S)-4-tert-butoxycarbonyl-3-(cyanomethyl)piperazin-1-yl]-6-chloro-8-fluoro-7-(3-hydroxy-1-naphthyl)quinazolin-2-yl]oxymethyl]pyrrolidin-1-yl]butoxy]acetic Acid

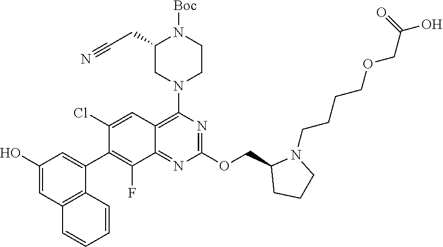 [SMILES: CC(C)(C)OC(=O)N1CCN(c2nc(OC[C@@H]3CCCN3CCCCOCC(=O)O)nc3c(F)c(-c4cc(O)cc5ccccc45)c(Cl)cc23)C[C@@H]1CC#N]

To a solution of tert-butyl (2S)-4-[2-[[(2S)-1-[4-(2-tert-butoxy-2-oxo-ethoxy)butyl]pyrrolidin-2-yl]methoxy]-6-chloro-8-fluoro-7-(3-hydroxy-1-naphthyl)quinazolin-4-yl]-2-(cyanomethyl)piperazine-1-carboxylate (140 mg, 0.168 mmol, 1 eq) in THF (1 mL) and CH₃OH (1 mL) was added a solution of LiOH.H₂O (141.0 mg, 3.36 mmol, 20 eq) in H₂O (3 mL), and the reaction mixture was stirred at 20° C. for 1.5 hours. The mixture was adjusted to pH=7 with aqueous HCl (2 N) and then concentrated in vacuum. The resulting residue was acidified to pH=3-4 with aqueous HCl (2 N) and then lyophilized to afford 2-[4-[(2S)-2-[[4-[(3S)-4-tert-butoxycarbonyl-3-(cyanomethyl)piperazin-1-yl]-6-chloro-8-fluoro-7-(3-hydroxy-1-naphthyl)quinazolin-2-yl]oxymethyl]pyrrolidin-1-yl]butoxy]acetic acid (130 mg, 95% yield) as a yellow solid. LC/MS (ESI) m/z: 777.2 [M+H]⁺.

### Step 9: Preparation of (2S,4R)-1-[(2S)-2-[[2-[4-[(2S)-2-[[6-chloro-4-[(3S)-3-(cyanomethyl)- 4-prop-2-enoyl-piperazin-1-yl]-8-fluoro-7-(3-hydroxy-1-naphthyl)quinazolin-2-yl]oxymethyl]pyrrolidin-1-yl]butoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[2-[4-[(2S)-2-[[6-Chloro-4-[(3S)-3-(cyanomethyl)-4-prop-2-enoyl- piperazin-1-yl]-8-fluoro-7-(3-hydroxy-1-naphthyl)quinazolin-2-yl]oxymethyl]pyrrolidin-1-yl]butoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[[2-[[1-[2-[6-chloro-8-fluoro-7-(3-hydroxy-1-naphthyl)-4-[(2S)-2-methyl-4-prop-2-enoyl-piperazin-1-yl]quinazolin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide starting from 2-[4-[(2S)-2-[[4-[(3S)-4-tert-butoxycarbonyl-3-(cyanomethyl)piperazin-1-yl]-6-chloro-8-fluoro-7-(3-hydroxy-1-naphthyl)quinazolin-2-yl]oxymethyl]pyrrolidin-1-yl]butoxy]acetic acid.

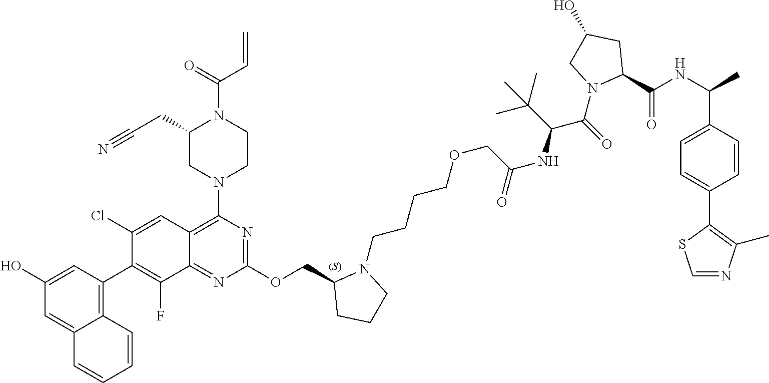

(free base, white solid). LC/MS (ESI) m/z: 1157.8 [M+H]⁺. ¹H-NMR (400 MHz, CDCl₃) δ 8.75-8.65 (m, 1H), 7.73 (dd, J=8.4, 4.0 Hz, 1H), 7.62 (s, 1H), 7.44-7.29 (m, 7H), 7.25-7.09 (m, 3H), 6.71-6.51 (m, 1H), 6.41 (d, J=16.4 Hz, 1H), 5.84 (d, J=10.0 Hz, 1H), 5.34-4.98 (m, 2H), 4.81-4.66 (m, 1H), 4.61-4.44 (m, 2H), 4.43-4.05 (m, 4H), 3.85-3.29 (m, 14H), 3.07-2.76 (m, 2H), 2.57-2.40 (m, 4H), 2.19-1.91 (m, 5H), 1.81-1.52 (m, 8H), 1.50-1.39 (m, 3H), 1.03 (s, 9H).

### Exemplary Synthesis of (2S,4R)-1-[(2S)-2-[[1-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]cyclopropanecarbonyl]amino]-3,3-dimethyl-butanoyl]-4-hydroxy-N-[(1S)-1-[4-(4-methylthiazol-5-yl)phenyl]ethyl]pyrrolidine-2-carboxamide

### Step 1: Preparation of tert-butyl 4-(trifluoromethylsulfonyloxymethyl)piperidine-1-carboxylate

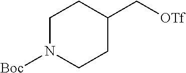 [SMILES: CC(C)(C)OC(=O)N1CCC(COS(=O)(=O)C(F)(F)F)CC1]

To a solution of tert-butyl 4-(hydroxymethyl)piperidine-1-carboxylate (200 mg, 0.929 mmol, 1 eq) and 2,6-dimethylpyridine (109 mg, 1.02 mmol, 1.1 eq) in CH₂Cl₂ (20 mL) at −78° C. was added Tf₂O (288 mg, 1.02 mmol, 1.1 eq) dropwise, and the reaction mixture was stirred at −78° C. for 1 hour. The reaction mixture was diluted with CH₂Cl₂ (25 mL) and washed sequentially with saturated NH₄Cl (25 mL) and water (25 mL). The organic layer was dried over anhydrous Na₂SO₄, filtered, and evaporated to afford tert-butyl 4-(trifluoromethylsulfonyloxymethyl)piperidine-1-carboxylate (321 mg, 0.924 mmol, 99.5% yield) as a red oil. ¹H-NMR (400 MHz, CDCl₃) δ 4.46-3.92 (m, 4H), 2.80-2.62 (m, 2H), 2.10-1.90 (m, 1H), 1.81-1.71 (m, 2H), 1.47 (s, 9H), 1.32-1.18 (m, 2H).

### Step 2: Preparation of 1-[(1-tert-butoxycarbonyl-4-piperidyl)methoxy]cyclopropanecarboxylic Acid

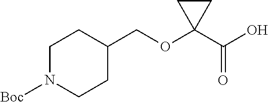 [SMILES: CC(C)(C)OC(=O)N1CCC(COC2(C(=O)O)CC2)CC1]

To a solution of ethyl 1-hydroxycyclopropanecarboxylate (100 mg, 0.768 mmol, 1 eq) in THF (5 mL) at −78° C. was added LiHMDS (1 M, 0.922 mL, 1.2 eq), and the reaction mixture was stirred at −78° C. under N₂ for 1 hour. A solution of tert-butyl 4-(trifluoromethylsulfonyloxymethyl)piperidine-1-carboxylate (320 mg, 0.922 mmol, 1.2 eq) in THF (5 mL) was then added at −78° C., and the reaction mixture was warmed to 20° C. and stirred at 20° C. for 16 hours. The reaction mixture was quenched by addition of saturated aqueous NH₄Cl (20 mL), and the resulting aqueous mixture was extracted with EtOAc (3×20 mL). The combined organic extract was washed with brine (2×20 mL), dried over anhydrous Na₂SO₄, filtered, and concentrated. To the resulting residue in CH₃OH (2 mL), THF (2 mL), and water (2 mL) was added LiOH.H₂O (98 mg, 2.3 mmol, 3 eq), and the reaction mixture was stirred at 50° C. under N₂ for 16 hours. The reaction mixture was concentrated under reduced pressure to give a residue. Water (20 mL) was then added, and the resulting mixture was extracted with EtOAc (3×10 mL). The organic extract was discarded, and the aqueous layer was adjusted to pH=5 with 2 N aqueous HCl and lyophilized to give 1-[(1-tert-butoxycarbonyl-4-piperidyl)methoxy]cyclopropanecarboxylic acid (94 mg, crude) as a red oil.

### Step 3: Preparation of tert-butyl 4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]piperidine-1-carboxylate

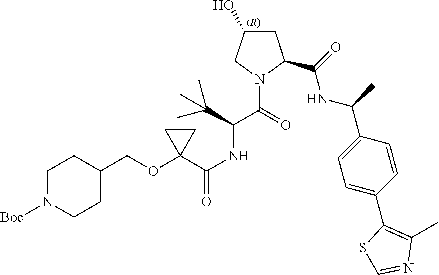

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (150 mg, 0.312 mmol, 1 eq, HCl) and 1-[(1-tert-butoxycarbonyl-4-piperidyl)methoxy]cyclopropanecarboxylic acid (93 mg, 0.312 mmol, 1 eq) in DMF (2 mL) were added diisopropylethylamine (202 mg, 1.56 mmol, 5 eq) and HATU (178 mg, 0.468 mmol, 1.5 eq), and the reaction mixture was stirred at 20° C. for 2 hours. The reaction mixture was quenched by addition of water (50 mL) and then stirred at 20° C. for 10 minutes. The resulting suspension was filtered, and the cake was dissolved in CH₂Cl₂ (30 mL). The organic layer was dried over anhydrous Na₂SO₄, filtered, and concentrated. The resulting crude product was purified by prep-TLC (acidic silica gel, CH₃OH/CH₂Cl₂=1:10) to give tert-butyl 4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]piperidine-1-carboxylate (81 mg, 0.11 mmol, 36% yield) as a yellow solid. LC/MS (ESI) m/z: 726.4 [M+H]⁺.

### Step 4: Preparation of (2S,4R)-1-[(2S)-3,3-dimethyl-2-[[1-(4-piperidylmethoxy)cyclopropanecarbonyl]amino]butanoyl]-4-hydroxy-N-[(1S)-1-[4-(4-methylthiazol-5-yl)phenyl]ethyl]pyrrolidine-2-carboxamide

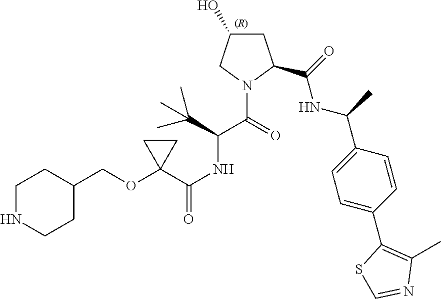

A solution of tert-butyl 4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]piperidine-1-carboxylate (81 mg, 0.11 mmol, 1 eq) in CH₂Cl₂ (1.5 mL) and TFA (0.5 mL) was stirred at 20° C. for 2 hours. The reaction mixture was concentrated under reduced pressure, and the resulting residue was dissolved in water (5 mL). The resulting aqueous mixture was adjusted to pH=9 with saturated Na₂CO₃ solution and extracted with 1:10 CH₃OH/CH₂Cl₂ (3×30 mL). The combined organic extract was washed with brine (2×30 mL), dried over anhydrous Na₂SO₄, filtered, and concentrated under reduced pressure to give (2S,4R)-1-[(2S)-3,3-dimethyl-2-[[1-(4-piperidylmethoxy)cyclopropanecarbonyl]amino]butanoyl]-4-hydroxy-N-[(1S)-1-[4-(4-methylthiazol-5-yl)phenyl]ethyl]pyrrolidine-2-carboxamide (65 mg, 0.10 mmol, 93% yield) as a yellow solid. LC/MS (ESI) m/z: 626.4 [M+H]⁺.

### Step 5: Preparation of (2S,4R)-1-[(2S)-2-[[1-[[1-[2-[6-chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]cyclopropanecarbonyl]amino]-3,3-dimethyl-butanoyl]-4-hydroxy-N-[(1S)-1-[4-(4-methylthiazol-5-yl)phenyl]ethyl]pyrrolidine-2-carboxamide

(2S,4R)-1-[(2S)-2-[[1-[[1-[2-[6-Chloro-4-[(3S)-3-(cyanomethyl)-4-prop-2-enoyl-piperazin-1-yl]-8-fluoro-7-(3-hydroxy-1-naphthyl)quinazolin-2-yl]oxyethyl]-4-piperidyl]methoxy]cyclopropanecarbonyl]amino]-3,3-dimethyl-butanoyl]-4-hydroxy-N-[(1S)-1-[4-(4-methylthiazol-5-yl)phenyl]ethyl]pyrrolidine-2-carboxamide was prepared in an analogous manner to (2S,4R)-1-[(2S)-2-[2-({1-[2-({6-chloro-4-[(3S)-3-(cyanomethyl)-4-(prop-2-enoyl)piperazin-1-yl]-8-fluoro-7-(3-hydroxynaphthalen-1-yl)quinazolin-2-yl}oxy)ethyl]piperidin-4-yl}methoxy)acetamido]-3,3-dimethylbutanoyl]-4-hydroxy-N-[(1S)-1-[4-(4-methyl-1,3-thiazol-5-yl)phenyl]ethyl]pyrrolidine-2-carboxamide starting from (2S,4R)-1-[(2S)-3,3-dimethyl-2-[[1-(4-piperidylmethoxy)cyclopropanecarbonyl]amino]butanoyl]-4-hydroxy-N-[(1S)-1-[4-(4-methylthiazol-5-yl)phenyl]ethyl]pyrrolidine-2-carboxamide.

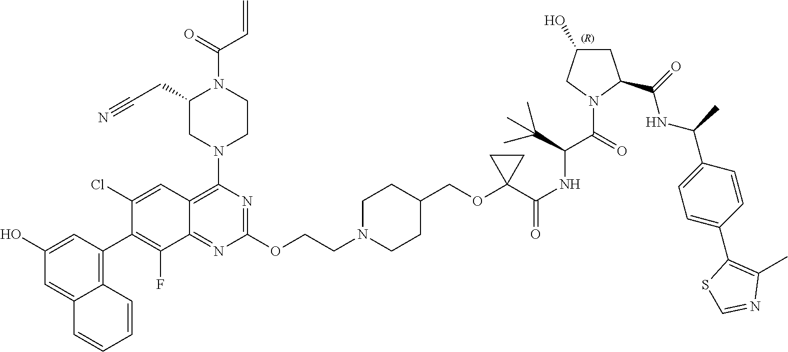

(free base, white solid). LC/MS (ESI) m/z: 1169.5 [M+H]⁺. ¹H-NMR (400 MHz, CD₃OD) δ 8.86 (s, 1H), 8.05 (s, 1H), 7.74 (d, J=8.8 Hz, 1H), 7.55-7.35 (m, 5H), 7.28-7.17 (m, 3H), 7.05-7.00 (m, 1H), 6.94-6.75 (m, 1H), 6.36-6.26 (m, 1H), 5.90-5.79 (m, 1H), 5.18-5.04 (m, 1H), 5.00-4.95 (m, 1H), 4.70-4.62 (m, 4H), 4.52-4.40 (m, 3H), 3.93-3.67 (m, 4H), 3.40 (d, J=6.0 Hz, 2H), 3.21-3.14 (m, 2H), 3.09-2.82 (m, 4H), 2.46 (s, 3H), 2.34-2.15 (m, 3H), 2.01-1.90 (m, 1H), 1.87-1.64 (m, 3H), 1.56-1.27 (m, 6H), 1.25-0.97 (m, 14H).

### Synthesis of 3

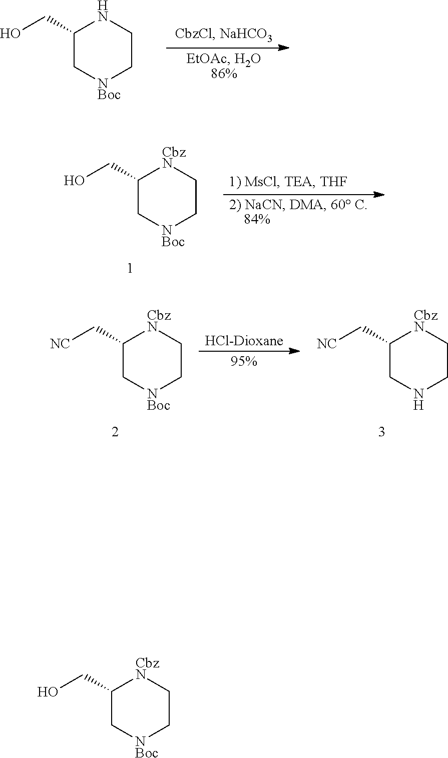

### 1-benzyl 4-(tert-butyl) (R)-2-(hydroxymethyl)piperazine-1,4-dicarboxylate (1)

To a solution of tert-butyl (3R)-3-(hydroxymethyl)piperazine-1-carboxylate (5.0 g, 1.0 eq) in Ethyl acetate (100 mL) was added NaHCO₃ (3.0 eq), H₂O (50 mL) and benzyl carbonochloridate (1.30 eq). The mixture was stirred at 25° C. for 12 hour. After completion, the organic phase was separated, washed with water (100 mL×2) dried over Na₂SO₄ and filtered. The solvent was removed under vacuum to give the title compound (7.0 g, 86% yield) as a yellow oil, which was used in the next step without further purification.

LCMS [ESI, M+1]: 351.

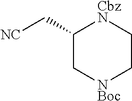 [SMILES: CC(C)(C)OC(=O)N1CCN(C(=O)OCc2ccccc2)[C@@H](CC#N)C1]

### 1-benzyl 4-(tert-butyl) (S)-2-(cyanomethyl)piperazine-1,4-dicarboxylate (2)

To a solution of I-benzyl 4-tert-butyl (2R)-2-(hydroxymethyl) piperazine-1,4-dicarboxylate (7.0 g, 1.0 eq) in THF (100 mL) was added TEA (3.0 eq) and methanesulfonyl chloride (1.2 eq). The mixture was stirred at 20° C. for one hour. The reaction mixture was quenched by addition H₂O 50 mL at 20° C. The reaction mixture was extracted with ethyl acetate (100 mL×2). The organic layers were washed with H₂O (100 mL), dried over Na₂SO₄, and filtered. The solvent was removed under vacuum. 1-benzyl 4-tert-butyl (2R)-2-(methylsulfonyloxymethyl) piperazine-1,4-dicarboxylate was obtained as a yellow oil. The crude product was used directly to the next step without further purification.

To a solution of 1-benzyl 4-tert-butyl (2R)-2-(methylsulfonyloxymethyl)piperazine-1,4-dicarboxylate in DMA (150 mL) was added NaCN (4 eq.). The mixture was stirred at 60° C. for 12 hour. The solvent was removed under vacuum to give an oil residue. The residue was diluted with H₂O (40 mL) and extracted with ethyl acetate (50 mL×3). The combined organic layers were washed with saturated brine, dried over Na₂SO₄, filtered and concentrated under reduced pressure to give a residue. The residue was purified by column chromatography (SiO₂, Hexanes/Ethyl acetate=5:1 to 3:1) to give the title compound (6.0 g, two steps yield 84%) as a yellow oil.

¹H NMR (600 MHz, CDCl₃) δ 7.40-7.32 (m, 5H), 5.16 (s, 2H), 4.55 (s, 1H), 4.25-3.80 (m, 3H), 2.95-3.25 (m, 2H), 2.85 (s, 1H), 2.60 (d, J=50.2 Hz, 1H), 2.74-2.40 (m, 2H).

¹³C NMR (150 MHz, CDCl₃) δ 154.9, 154.6, 135.8, 128.6, 128.4, 128.2, 116.7, 81.0, 68.0, 48.2, 45.3, 42.4, 39.2, 28.3, 19.1.

HRMS [C₁₉H₂₅N₃O₄Na⁺] Cal: 382.1737; Obs: 382.1743.

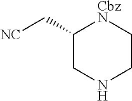 [SMILES: N#CC[C@H]1CNCCN1C(=O)OCc1ccccc1]

### benzyl (S)-2-(cyanomethyl)piperazine-1-carboxylate (3)

To a solution of I-benzyl 4-tert-butyl (2S)-2-(cyanomethyl) piperazine-1,4-dicarboxylate (6.0 g, 1.0 eq) in dioxane (20.8 mL) was added 4.0 M HCl in dioxane (20.8 mL, 5.0 eq). The mixture was stirred at 20° C. for 1 hour. Then NaHCO₃ was added to the reaction mixture until a pH>7 was reached, after which the reaction was concentrated under reduced pressure to remove dioxane. The residue was diluted with H₂O (50 mL) and extracted with ethyl acetate (50 mL×3). The combined organic layers were washed with H₂O (20 mL), dried over Na₂SO₄, filtered and concentrated under reduced pressure to give a residue. The product benzyl (2S)-2-(cyanomethyl) piperazine-1-carboxylate (4.1 g, 95% yield) was obtained as a yellow oil.

### Synthesis of 15

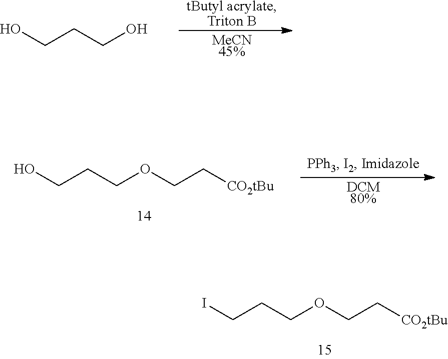

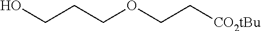 [SMILES: CC(C)(C)OC(=O)CCOCCCO]

### tert-butyl 3-(3-hydroxypropoxy)propanoate (14)

To a solution of propane 1,3-diol (20 mmol, 1.5 g, 1.0 eq.) in acetonitrile (20 mL) was added Triton B (0.3 eq.), and tert-butyl 3-(3-hydroxypropoxy)propanoate (1.0 eq.). The mixture was stirred at 20° C. overnight and concentrated under vacuum. The residue was purified by column chromatography (SiO₂, Hexanes/Ethyl acetate=5:1 to 1:1) to give the title compound as a colorless oil (1.85 g, 45% yield).

¹H NMR (600 MHz, CDCl₃) δ 3.77-3.72 (m, 2H), 3.67 (t, J=6.2 Hz, 2H), 3.65-3.60 (m, 2H), 2.48 (t, J=6.2 Hz, 2H), 1.83-1.78 (m, 2H), 1.44 (s, 9H).

¹³C NMR (150 MHz, CDCl₃) δ 171.1, 80.8, 70.1, 66.7, 61.8, 36.2, 31.9, 28.0.

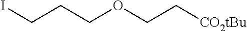 [SMILES: CC(C)(C)OC(=O)CCOCCCI]

### tert-butyl 3-(3-iodopropoxy)propanoate (15)

14 (1.85 g, 1.0 eq.) was dissolved in dichloromethane (30 mL) and triphenylphosphine (1.1 eq.) was added followed by imidazole (1.2 eq.). 12 (1.1 eq.) was added portion wise and the reaction mixture was stirred overnight at 20° C. The reaction mixture was quenched with a saturated aqueous solution of sodium thiosulfate and stirred for 20 minutes. The organic layer was separated and the aqueous extracted with dichloromethane. The organic layers were combined, washed with brine, dried over Na₂SO₄ and concentrated in vacuo. The residue was purified by column chromatography (SiO₂, Hexanes/Ethyl acetate=0:1 to 3:1) to yield the title compound (2.01 g, 80% yield).

¹H NMR (600 MHz, CDCl₃) δ 3.66 (t, J=6.4 Hz, 2H), 3.48 (t, J=5.8 Hz, 2H), 3.25 (t, J=6.8 Hz, 2H), 2.46 (t, J=6.4 Hz, 2H), 2.03 (tt, J=6.9, 5.8 Hz, 2H), 1.44 (s, 9H).

¹³C NMR (150 MHz, CDCl₃) δ 170.8, 80.5, 70.1, 66.5, 36.3, 33.3, 28.1, 3.3.

HRMS [C₁₀H₁₉INaO₃⁺] Cal: 337.0271; Obs: 337.2077.

### Synthesis of 6

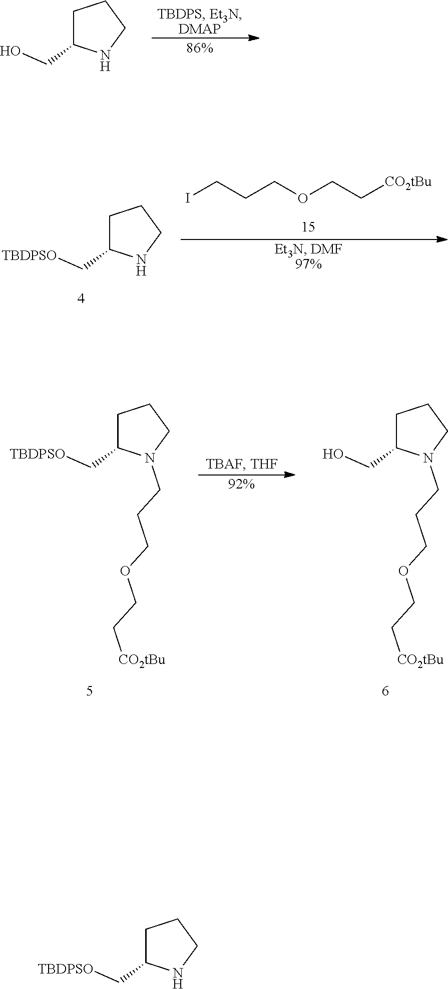

### (S)-2-(((tert-butyldiphenylsilyl)oxy)methyl)pyrrolidine (4)

To a solution of (S)-(+)-2-Pyrrolidinemethanol (1.0 g, 1.0 eq.) in THF was added tert-butyl-chloro-diphenyl-silane (1.2 eq.), DMAP (0.1 eq.), and TEA (3.0 eq.). The mixture was stirred at 20° C. overnight and concentrated under vacuum. The residue was purified by column chromatography (SiO₂, DCM/Methanol=1:0 to 10:1) to give the title compound as a yellow oil (2.9 g, 86% yield).

¹H NMR (600 MHz, CDCl₃) δ 7.68-7.65 (m, 4H), 7.44-7.35 (m, 6H), 3.68 (dd, J=10.3, 4.8 Hz, 1H), 3.61 (dd, J=10.1, 6.1 Hz, 1H), 3.32-3.26 (m, 1H), 3.04-2.98 (m, 1H), 2.93-2.87 (m, 1H), 1.84-1.70 (m, 3H), 1.55-1.47 (m, 1H), 1.06 (s, 9H).

¹³C NMR (150 MHz, CDCl₃) δ 135.6, 133.5, 133.4, 129.6, 127.7, 66.0, 59.9, 46.3, 27.4, 26.9, 25.16, 19.3.

HRMS [C₂₁H₃₀NOSi⁺] Cal: 340.2091; Obs: 340.2088.

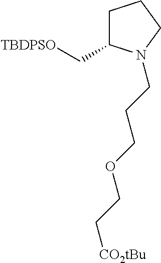

### tert-butyl (S)-3-(3-(2-(((tert-butyldiphenylsilyl)oxy)methyl)pyrrolidin-1-yl)propoxy)propanoate (5)

To a solution of 4 (2.0 g, 1.0 eq.) in DMF was added tert-butyl 3-(3-iodopropoxy)propanoate (1.0 eq.) and TEA (3.0 eq.). The mixture was stirred at 20° C. overnight and concentrated under vacuum. The residue was purified by column chromatography (SiO₂, DCM/Methanol=1:0 to 10:1) to give the title compound as a yellow oil (3.0 g, 97% yield).

¹H NMR (600 MHz, CDCl₃) δ 7.80-7.56 (m, 4H), 7.51-7.37 (m, 6H), 4.56-3.79 (m, 4H), 3.66-3.44 (m, 5H), 3.36 (s, 1H), 3.06 (s, 1H), 2.53-2.31 (m, 3H), 2.26-1.99 (m, 5H), 1.90-1.78 (m, 1H), 1.60 (s, 2H), 1.43 (s, 9H), 1.08 (s, 9H).

¹³C NMR (150 MHz, CDCl₃) δ 171.1, 135.7, 135.5, 130.2, 128.1, 128.0, 80.8, 70.5, 68.2, 66.4, 62.7, 54.9, 35.9, 28.1, 26.9, 25.5, 22.07, 19.1.

HRMS [C₃₁H₄₈NO₄Si⁺] Cal: 526.3347; Obs: 526.3353.

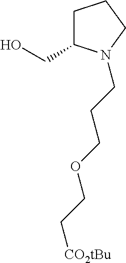 [SMILES: CC(C)(C)OC(=O)CCOCCCN1CCC[C@H]1CO]

### tert-butyl (S)-3-(3-(2-(hydroxymethyl)pyrrolidin-1-yl)propoxy)propanoate (6)

To a solution of 5 (3.0 g, 1.0 eq.) in THF was added TBAF (1.0 M in THF, 2.0 eq.). The mixture was stirred at 20° C. overnight and concentrated under vacuum. The residue was purified by column chromatography (SiO₂, DCM/Methanol=1:0 to 5:1) to give the title compound as a yellow oil (1.5 g, 92% yield).

HRMS [C₁₅H₃₀NO₄⁺] Cal: 288.2169; Obs: 288.2175.

### Synthesis of LC-2 (US 2018/0072723 A1)

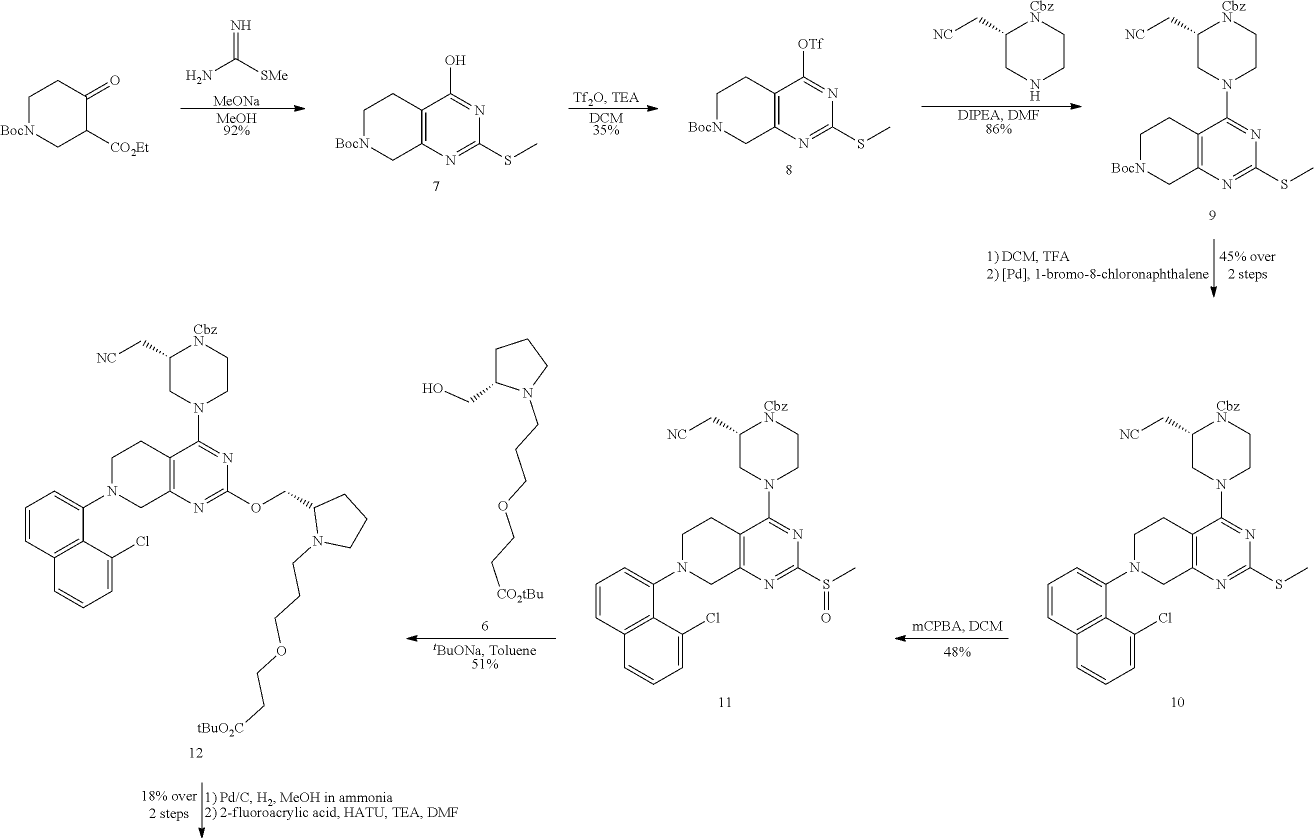

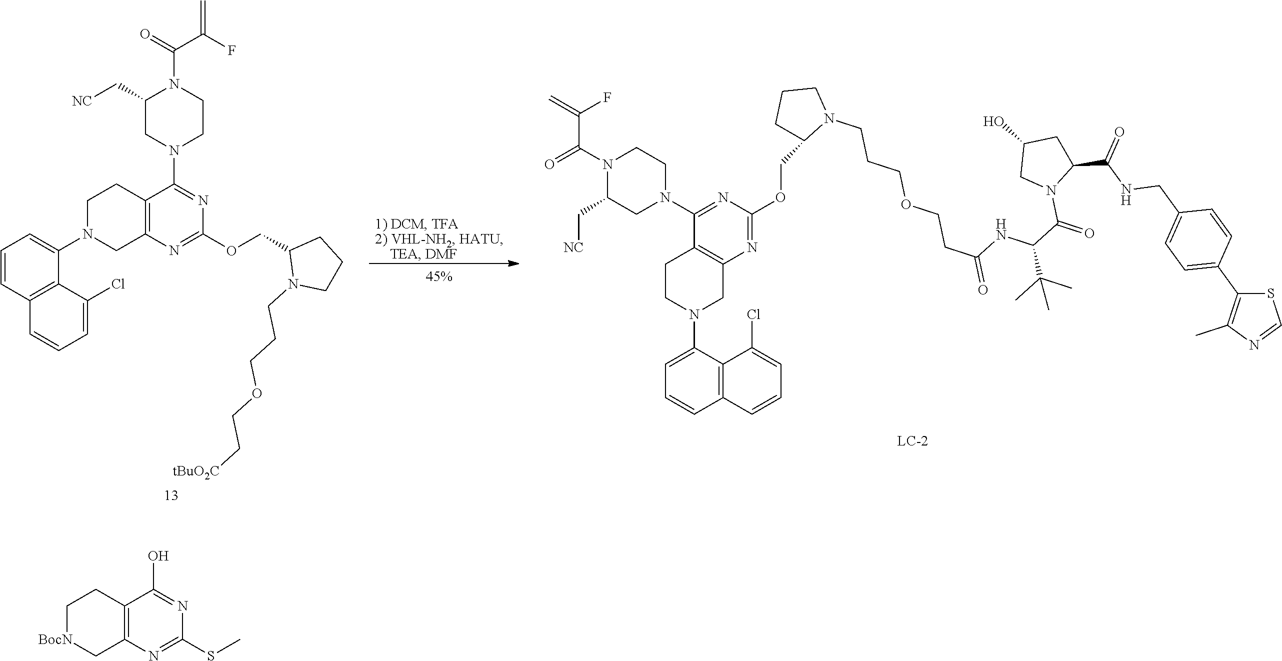

### tert-butyl 4-hydroxy-2-(methylthio)-5,8-dihydropyrido[3,4-d]pyrimidine-7(6H)-carboxylate (7)

Step 1: To a stirred solution of 1-tert-butyl 4-ethyl 3-oxopiperidine-1,4-dicarboxylate (17.0 g, 1.0 eq) in MeOH (300 mL) at 20° C. under nitrogen was added NaOMe (5.0 eq), followed by 2-methylisothiourea (1.80 eq.) as a solid. The reaction mixture was stirred at 20° C. for 16 hours. The reaction mixture was acidified with HCl (2 M) until pH-5, and then the mixture was concentrated under reduced pressure to removed MeOH. The residue was resuspended in 300 mL of ethyl acetate and 300 mL of water and stirred rapidly. The suspension was filtered and the white solid was collected. The filtrate was separated and the organics washed with water (1×300 mL) and brine (1×200 mL). The organics were isolated, dried over Na₂SO₄, filtered and concentrated to a white solid, tert-butyl 4-hydroxy-2-methylsulfanyl-6,8-dihydro-5H-pyrido[3,4-d]pyrimidine-7-carboxylate (17.2 g, 92% yield) was obtained as a white solid and used directly for next step without further purification.

LCMS [M+1]: 298.

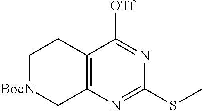 [SMILES: CSc1nc2c(c(OS(=O)(=O)C(F)(F)F)n1)CCN(C(=O)OC(C)(C)C)C2]

### tert-butyl 2-(methylthio)-4-(((trifluoromethyl)sulfonyl)oxy)-5,8-dihydropyrido[3,4-d]pyrimidine-7(6H)-carboxylate (8)

Step 2: To a stirred suspension of 7 (10 g, 1.0 eq) in DCM (200 mL) at 0° C. was added DIEA (2.0 eq.) followed by Tf₂O (1.5 eq.) under nitrogen. Immediately a brown solution formed. After stirring at 25° C. for 16 hours, the reaction was concentrated to give a brown oil. The brown oil was purified by column chromatography (SiO₂, Hexanes/Ethyl acetate=1/0 to 10/1) to give the title compound (5.1 g, 35% yield) as a yellow solid.

HRMS [C₁₄H₁₉F₃N₃O₅S₂+] Cal: 430.0713; Obs: 430.0718.

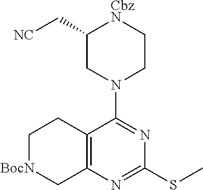 [SMILES: CSc1nc2c(c(N3CCN(C(=O)OCc4ccccc4)[C@@H](CC#N)C3)n1)CCN(C(=O)OC(C)(C)C)C2]

### tert-butyl (S)-4-(4-((benzyloxy)carbonyl)-3-(cyanomethyl)piperazin-1-yl)-2-(methylthio)-5,8-dihydropyrido[3,4-d]pyrimidine-7(6H)-carboxylate (9)

Step 3: A mixture of 8 (1.24 g, 1.0 eq), benzyl-(2S)-2-(cyanomethyl)piperazine-1-carboxylate (1.05 eq), and DIEA (3.0 eq) in DMF (10 mL) was degassed and purged with N₂ 3 times, and then the mixture was stirred at 100° C. for 1 hour under N₂ atmosphere. After completion, the solvent was removed under vacuum. The residue was purified by column chromatography (SiO₂, Hexanes/Ethyl acetate=3/1 to 1:1) to give title compound (1.36 g, 86% yield) as a yellow solid.

¹H NMR (600 MHz, CDCl₃) δ 7.43-7.30 (m, 5H), 5.17 (s, 2H), 4.72-4.55 (m, 2H), 4.36 (d, J=19.1 Hz, 1H), 4.02-3.71 (m, 3H), 3.40-3.21 (m, 4H), 2.98 (t, J=12.2 Hz, 1H), 2.75-2.57 (m, 4H), 2.49 (s, 3H), 1.47 (s, 9H).

¹³C NMR (151 MHz, CDCl₃) δ 168.61, 164.66, 135.82, 128.68, 128.65, 128.45, 128.29, 128.26, 128.23, 117.00, 111.12, 68.07, 60.39, 48.50, 47.84, 28.41, 28.02, 25.88, 21.05, 19.16, 17.59, 14.19, 14.05.

HRMS [C₂₇H₃₅N₆O₄S⁺] Cal: 539.2435; Obs: 539.2438.

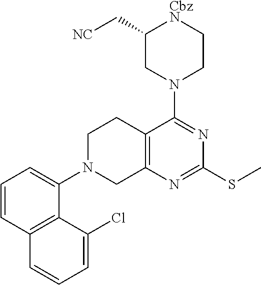 [SMILES: CSc1nc2c(c(N3CCN(C(=O)OCc4ccccc4)[C@@H](CC#N)C3)n1)CCN(c1cccc3cccc(Cl)c13)C2]

### benzyl (S)-4-(7-(8-chloronaphthalen-1-yl)-2-(methylthio)-5,6,7,8-tetrahydropyrido[3,4-d]pyrimidin-4-yl)-2-(cyanomethyl)piperazine-1-carboxylate (10)

Step 4: A mixture of 9 (1.36 g, 1.0 eq), TFA (6.8 mL) in DCM (6.8 mL) was degassed and purged with N₂ 3 times, and then the mixture was stirred at 20° C. for 1 hour under N₂ atmosphere. After completion, the reaction mixture was quenched with saturated NaHCO₃ solution. The mixture was extracted with ethyl acetate (3×50 mL) and the organic layer was dried over Na₂SO₄ and filtered. The solvent was removed under vacuum to give benzyl (S)-2-(cyanomethyl)-4-(2-(methylthio)-5,6,7,8-tetrahydropyrido[3,4-d]pyrimidin-4-yl)piperazine-1-carboxylate (1.11 g, crude) as a yellow solid which was used for the next step without further purification.

Step 5: A mixture of benzyl (2S)-2-(cyanomethyl)-4-(2-methylsulfanyl-5,6,7,8-tetrahydropyrido[3,4-d]pyrimidin-4-yl)piperazine-1-carboxylate (1.11 g, 1.0 eq.), 1-bromo-8-chloro-naphthalene (1.8 eq.), Pd₂(dba)₃ (0.1 eq.), RuPhos (0.2 eq.) and Cs₂CO₃ (3.6 eq.) in toluene (10 mL) was degassed and purged with N₂ 3 times, and then the mixture was stirred at 100° C. for 12 hours under N₂ atmosphere. After completion, the reaction mixture was filtered. The organic solvent was removed under vacuum to give an oil residue. The residue was purified by column chromatography (SiO₂, Hexanes/Ethyl acetate=5:1 to 3:1) to give the title compound (0.77 g, two steps yield 45%) as a dark yellow solid.

HRMS [C₃₂H₃₂ClN₆O₂S+] Cal: 599.1990; Obs: 599.1996.

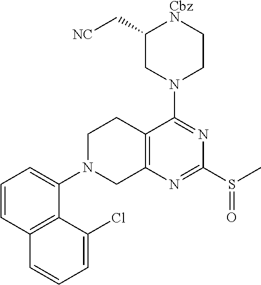 [SMILES: CS(=O)c1nc2c(c(N3CCN(C(=O)OCc4ccccc4)[C@@H](CC#N)C3)n1)CCN(c1cccc3cccc(Cl)c13)C2]

### benzyl (2S)-4-(7-(8-chloronaphthalen-1-yl)-2-(methylsulfinyl)-5,6,7,8-tetrahydropyrido[3,4-d]pyrimidin-4-yl)-2-(cyanomethyl)piperazine-1-carboxylate (11)

Step 6: A mixture of 10 (734 mg, 1.0 eq), m-CPBA (0.6 eq.) in DCM (8 mL) was stirred at 0° C. for 30 min. After which another batch of m-CPBA (0.6 eq.) was added and the mixture was stirred for another 30 min at 0° C. After completion, the reaction was quenched with water (10 mL). The mixture was extracted with ethyl acetate (3×10 mL). The combined organic layer was dried with Na₂SO₄ and filtered. The solvent was removed to give an oil residue. The residue was purified by column chromatography (SiO₂, Methanol/Ethyl acetate=0:1 to 1:10) to give the title compound (350 mg, 46% yield) as a yellow solid.

LC-MS [ESI, M+1]=616.

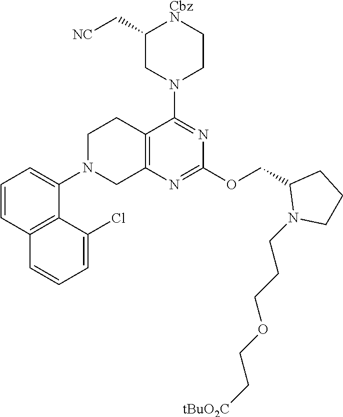

### benzyl (S)-4-(2-(((S)-1-(3-(3-(tert-butoxy)-3-oxopropoxy)propyl)pyrrolidin-2-yl)methoxy)-7-(8-chloronaphthalen-1-yl)-5,6,7,8-tetrahydropyrido[3,4-d]pyrimidin-4-yl)-2-(cyanomethyl)piperazine-1-carboxylate (12)

Step 7: To a solution of 11 (350 mg, 1.0 eq.) and tert-butyl (S)-3-(3-(2-(hydroxymethyl)pyrrolidin-1-yl)propoxy)propanoate (6) (3.0 eq.) in toluene (5 mL) was added t-BuONa (3.0 eq.). The mixture was stirred at 0° C. for 0.5 hour. After completion, the mixture was added to cold water (5 mL) and extracted with ethyl acetate (3×5 mL). The combined organic layer was dried over Na₂SO₄, filtered and concentrated. The obtained product was purified by column chromatography (SiO₂, Ethyl acetate:Methanol=1:0 to 10:1) to give the title compound (230 mg, 44% yield) as a yellow solid.

LC-MS [ESI, M+1]=838.

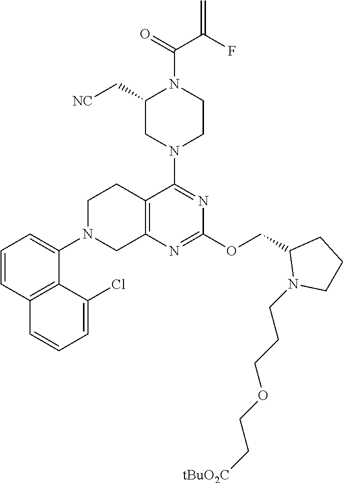

### tert-butyl 3-(3-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)propoxy)propanoate (13)

Step 8: To a solution of 12 (230 mg, 1.0 eq.) in MeOH (3 mL) was added 7N NH₃ in MeOH (3 mL), and Pd/C (100 mg, 10% purity) under N₂. The suspension was degassed under vacuum and purged with H₂ several times. The mixture was stirred under H₂ (15 psi) at 20° C. for 4 hours. After which another batch of Pd/C (100 mg, 10% purity) was added. The mixture was stirred under H₂ overnight. Upon completion, the catalyst was filtered off and the filtrate was concentrated under vacuum to give the title compound (100 mg, 52% yield) as a yellow solid which was used directly in the next step without further purification. LC-MS [ESI, M+1]=704.

Step 9: To a solution of above product (141 mg, 1.0 eq.) in DMF was added sodium 2-fluoroprop-2-enoyloxy (2.0 eq.), HATU (1.5 eq.), and TEA (4.0 eq.). The mixture was stirred at room temperature for 1 hour. After completion, the residue was diluted with H₂O (15 mL), extracted with EtOAc (3×15 mL), dried over Na₂SO₄, concentrated under vacuum and purified by Prep-TLC (DCM:Methanol:Ammonia=200:10:1). The title compound (42 mg, 27% yield) was obtained as a colorless oil.

LC-MS [ESI, M+1]=776.

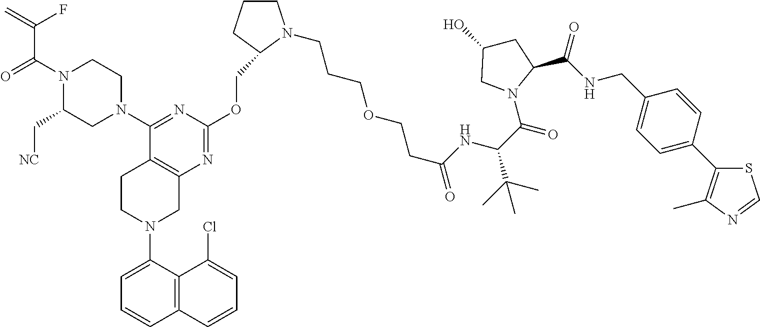 [SMILES: C=C(F)C(=O)N1CCN(c2nc(OC[C@@H]3CCCN3CCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N]

### (2S,4R)-1-((S)-2-(3-(3-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)propoxy)propanamido)-3,3-dimethylbutanoyl)-4-hydroxy-N-(4-(4-methylthiazol-5-yl)benzyl)pyrrolidine-2-carboxamide (LC-2)

Step 10: To a solution of 13 (21 mg, 1.0 eq.) in DCM (1 mL) was added TFA (1 mL). The mixture was stirred for 0.5 hour at room temperature, concentrated under vacuum and used in the next step without further purification.

To a solution of above product (1.0 eq.) in DMF (1 mL) was added (1R)-1-[(2S,4R)-4-hydroxy-2-[[4-(4-methylthiazol-5-yl)phenyl]methylcarbamoyl]pyrrolidine-1-carbonyl]-2,2-dimethyl-propyl (1.2 eq.), HATU (1.3 eq.), and TEA (5.0 eq.). The mixture was stirred for 1 hour at room temperature. Upon completion, the mixture was diluted with H₂O (5 mL), extracted with EtOAc (3×5 mL), dried over Na₂SO₄, concentrated under vacuum and purified by reverse phase HPLC. The title compound (10 mg, 33% yield) was obtained as a colorless oil.

¹H NMR (600 MHz, Acetone-d₆) δ 8.82 (s, 1H), 7.86 (t, J=7.9 Hz, 2H), 7.70 (dd, J=8.0, 3.9 Hz, 1H), 7.55 (dd, J=7.3, 3.6 Hz, 1H), 7.53-7.48 (m, 1H), 7.45 (dd, J=7.8, 4.2 Hz, 2H), 7.43-7.39 (m, 1H), 7.39-7.33 (m, 3H), 7.29-7.24 (m, 1H), 5.33-5.20 (m, 2H), 4.64 (dd, J=9.3, 1.6 Hz, 1H), 4.60-4.48 (m, 3H), 4.39-4.27 (m, 3H), 4.24-4.08 (m, 2H), 3.87-3.78 (m, 2H), 3.78-3.70 (m, 2H), 3.67-3.53 (m, 3H), 3.53-3.43 (m, 3H), 3.37 (dd, J=13.8, 3.8 Hz, 1H), 3.34-3.24 (m, 2H), 3.25-3.12 (m, 4H), 3.13-2.96 (m, 4H), 2.81-2.75 (m, 1H), 2.71-2.65 (m, 1H), 2.48-2.39 (m, 4H), 2.39-2.32 (m, 1H), 2.26-2.18 (m, 1H), 2.18-2.11 (m, 1H), 2.11-2.06 (m, 1H), 1.91-1.82 (m, 1H), 1.79-1.61 (m, 6H), 0.96 (s, 9H).

¹³C NMR (151 MHz, Acetone-d₆) δ 172.63, 171.41, 171.34, 170.96, 167.67, 166.06, 163.77, 157.79 (d, J=269.1 Hz), 151.29, 149.67, 149.41, 149.18, 140.54, 138.50, 132.37, 131.35, 130.64, 130.53, 129.92, 129.43, 128.76, 127.78, 126.77, 126.71, 125.92 (d, J=11.3 Hz), 119.93, 118.28, 110.17, 77.33, 70.71, 69.85, 69.83, 67.74, 63.39, 60.60, 59.97, 59.75, 57.59, 57.46, 54.95, 53.23, 51.27, 51.21, 43.20, 38.44, 38.43, 37.41, 36.50, 32.70, 26.98, 26.86, 26.41, 24.05, 23.39, 20.89, 19.43, 16.41, 14.56, 14.41.

HRMS [C₅₉H₇₂ClFN₁₁O₇S⁺] Cal: 1132.5004; Obs: 1132.5010.

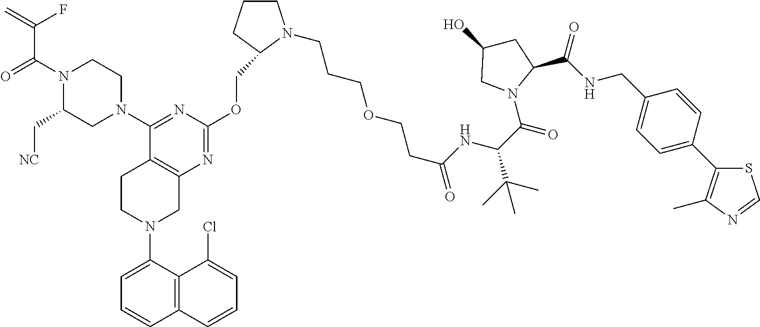 [SMILES: C=C(F)C(=O)N1CCN(c2nc(OC[C@@H]3CCCN3CCCOCCC(=O)N[C@H](C(=O)N3C[C@@H](O)C[C@H]3C(=O)NCc3ccc(-c4scnc4C)cc3)C(C)(C)C)nc3c2CCN(c2cccc4cccc(Cl)c24)C3)C[C@@H]1CC#N]

### (2S,4S)-1-((S)-2-(3-(3-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)propoxy)propanamido)-3,3-dimethylbutanoyl)-4-hydroxy-N-(4-(4-methylthiazol-5-yl)benzyl)pyrrolidine-2-carboxamide (LC-2-Epimer)

To a solution of 13 (21 mg, 1.0 eq.) in DCM (1 mL) was added TFA (1 mL). The mixture was stirred for 0.5 hour at room temperature, concentrated under vacuum and used in the next step without further purification.

To a solution of above product (1.0 eq.) in DMF (1 mL) was added (1R)-1-[(2S,4S)-4-hydroxy-2-[[4-(4-methylthiazol-5-yl)phenyl]methylcarbamoyl]pyrrolidine-1-carbonyl]-2,2-dimethyl-propyl (1.2 eq.), HATU (1.3 eq.), and TEA (5.0 eq.). The mixture was stirred for 1 hour at room temperature. Upon completion, the mixture was diluted with H₂O (5 mL), extracted with EtOAc (3×5 mL), dried over Na₂SO₄, concentrated under vacuum and purified by reverse phase HPLC. The title compound (4.4 mg, 15% yield) was obtained as a colorless oil.

¹H NMR (400 MHz, Acetone-d₆) δ 8.82 (s, 1H), 7.87 (d, J=8.5 Hz, 1H), 7.70 (dd, J=8.1, 2.6 Hz, 1H), 7.59-7.53 (m, 1H), 7.52-7.32 (m, 7H), 7.24 (dd, J=9.4, 3.3 Hz, 1H), 5.37-5.17 (m, 2H), 5.00-4.83 (m, 1H), 4.67-4.57 (m, 2H), 4.51 (d, J=9.6 Hz, 1H), 4.40-4.22 (m, 4H), 4.21-3.95 (m, 4H), 3.87-3.71 (m, 4H), 3.66-3.46 (m, 7H), 3.33-3.25 (m, 2H), 3.23-3.11 (m, 2H), 2.83-2.62 (m, 4H), 2.53-2.33 (m, 6H), 2.32-2.24 (m, 2H), 2.22-2.11 (m, 2H), 1.95 (d, J=2.8 Hz, 1H), 1.91 (s, 2H), 1.78-1.62 (m, 4H), 0.97 (s, 9H).

HRMS [C₅₉H₇₂ClFN₁₁O₇S⁺] Cal: 1132.5004; Obs: 1132.4999.

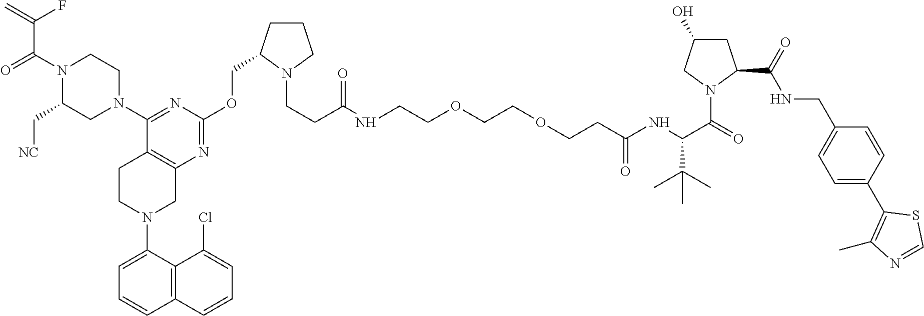 [SMILES: C=C(F)C(=O)N1CCN(c2nc(OC[C@@H]3CCCN3CCC(=O)NCCOCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N]

### (2S,4R)-1-((S)-2-(tert-butyl)-16-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)-4,14-dioxo-7,10-dioxa-3,13-diazahexadecanoyl)-4-hydroxy-N-(4-(4-methylthiazol-5-yl)benzyl)pyrrolidine-2-carboxamide (LC-1)

LC-1 was synthesized using the same procedure as for LC-2 except that in step 7, tert-butyl (S)-3-(2-(hydroxymethyl)pyrrolidin-1-yl)propanoate was used instead of tert-butyl (S)-3-(3-(2-(hydroxymethyl)pyrrolidin-1-yl)propoxy)propanoate (6), and in step 10, (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide was used instead of (1R)-1-[(2S,4S)-4-hydroxy-2-[[4-(4-methylthiazol-5-yl)phenyl]methylcarbamoyl]pyrrolidine-1-carbonyl]-2,2-dimethyl-propyl.

¹H NMR (600 MHz, Methanol-d4) δ 8.84 (s, 1H), 7.80 (d, J=8.2 Hz, 1H), 7.65 (dd, J=8.2, 3.7 Hz, 1H), 7.52-7.49 (m, 1H), 7.46 (dd, J=7.8, 7.8 Hz, 1H), 7.43-7.40 (m, 2H), 7.39-7.33 (m, 3H), 7.32-7.27 (m, 1H), 5.38-5.23 (m, 2H), 4.63 (s, 1H), 4.55 (t, J=8.4 Hz, 1H), 4.48 (d, J=11.6 Hz, 2H), 4.41-4.36 (m, 1H), 4.35-4.24 (m, 4H), 4.16 (d, J=13.6 Hz, 1H), 4.11-4.02 (m, 2H), 3.87 (d, J=10.9 Hz, 1H), 3.77 (dd, J=11.0, 3.9 Hz, 1H), 3.72-3.52 (m, 5H), 3.46 (dt, J=7.4, 5.5 Hz, 2H), 3.38-3.27 (m, 9H), 3.25-3.04 (m, 6H), 2.91 (dd, J=17.1, 6.9 Hz, 1H), 2.79-2.58 (m, 2H), 2.54-2.36 (m, 8H), 2.23-2.16 (m, 1H), 2.10-2.02 (m, 2H), 1.90-1.71 (m, 3H), 1.40-1.25 (m, 3H), 0.99 (d, J=1.8 Hz, 9H).

¹³C NMR (151 MHz, Methanol-d4) δ 174.57, 174.51, 173.14, 173.07 (d, J=220.9 Hz), 167.96, 167.80, 166.82, 163.67, 152.99, 149.85 (d, J=21.0 Hz), 149.19, 140.42, 139.06, 133.53, 131.67, 131.08, 131.02, 130.94, 130.51, 129.73, 129.12, 127.96, 127.23, 126.96, 126.52, 126.49, 120.29, 111.58, 110.40, 71.20, 70.56, 69.91, 68.15, 64.89, 61.69, 61.01, 60.59, 60.44, 59.03, 58.23, 55.36, 52.77, 52.73, 51.29, 43.84, 40.51, 39.16, 37.78, 37.37, 36.98, 35.24, 30.93, 29.22, 27.64, 27.51, 27.13, 24.15, 21.01, 16.02, 14.61, 11.58.

HRMS [C₆₃H₇₉ClFN₁₂O₉S⁺] Cal: 1233.5481; Obs: 1233.5490.

Protein Level Control

This description also provides methods for the control of protein levels within a cell. The method is based on the use of compounds as described herein such that degradation of the target protein KRas in vivo will result in the reducing the amount of the target protein in a biological system, preferably to provide a particular therapeutic benefit.

The following examples are used to assist in describing the present disclosure, but should not be seen as limiting the present disclosure in any way.

In certain embodiments, the description provides the following exemplary KRas-degrading bifunctional molecules (compounds of Table 7 or exemplary compounds 1-172), including salts, polymorphs, analogs, derivatives, and deuterated forms thereof.

MRTX849 Based VHL-Recruiting Bifunctional Protein-Degradant Compound Engage and Degrade Endogenous KRAS^{G12C} in Homozygous and Heterozygous Mutant Cell Lines. MRTX849.

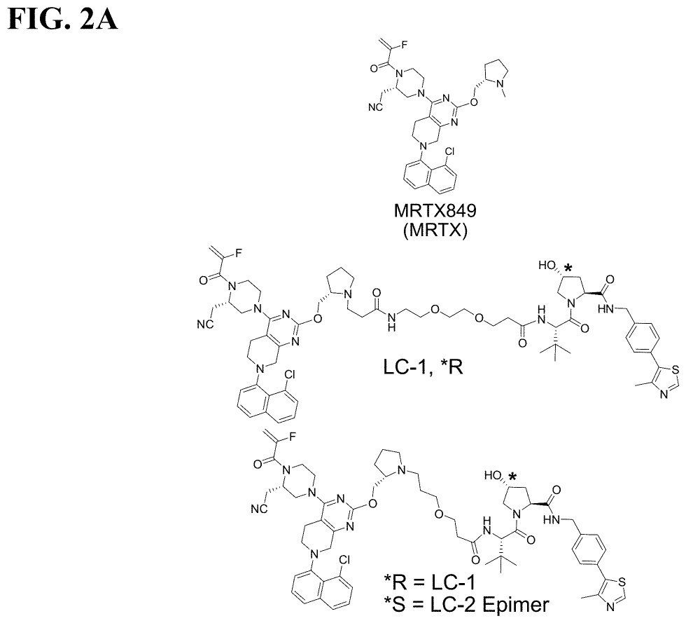

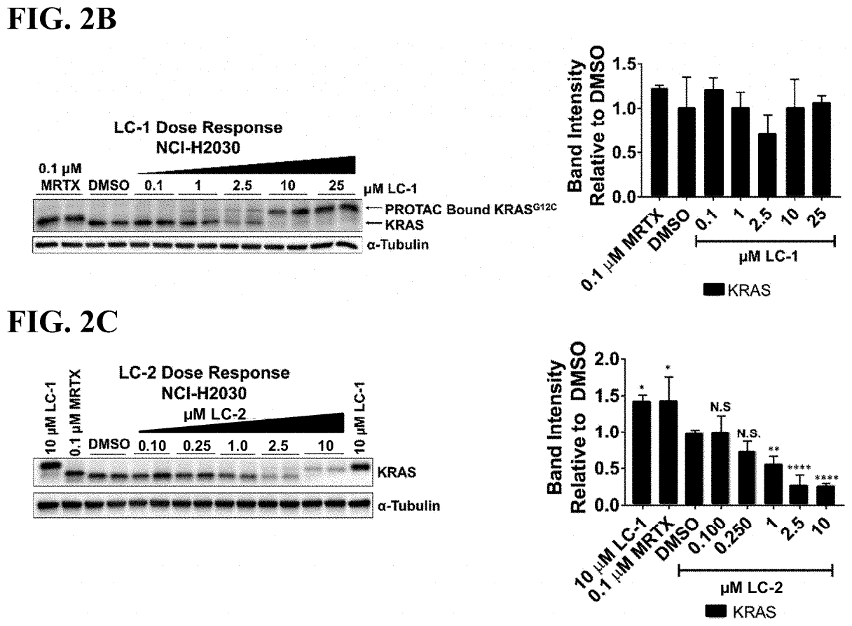

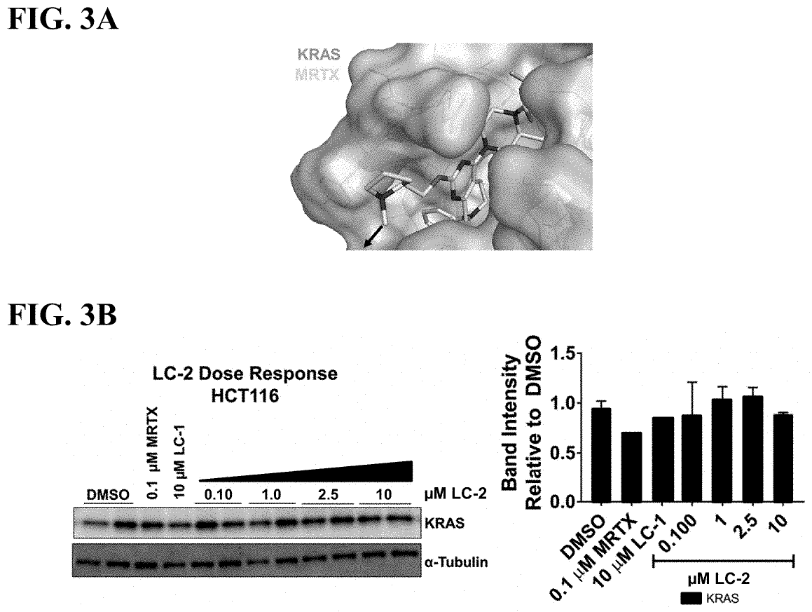

Docking of MRTX into the “switch II” pocket of KRAS^{G12C} reveals the pyrrolidine group to be solvent exposed (PDB: 5V9U; FIG. 3A) (Janes et al. 2018). To avoid introducing another stereocenter at the 2, 3 or 4 position of the pyrrolidine and further complicating the synthetic route, it was decided to build linkers from the N-methyl moiety of the pyrrolidine. The first evidence of KRAS engagement was observed with LC-1 (FIGS. 2A and 2B). When NCI-H2030 cells were treated with increasing concentrations of LC-1 for 24 hours we observed a clear band shift at 1, 2.5, 10, and 25 μM, indicating the presence of KRAS conjugated with the hetero-bifunctional compound (FIG. 2B). However, only a small, non-significant reduction in KRAS levels was observed. Therefore, these data indicate that LC-1 can engage KRAS^{G12C}, but does not efficiently degrade the protein. As a result, LC-1 was subsequently used as a positive control for KRAS engagement during bifunctional compound screens.

LC-1 has a hydrolysable amide within the linker. To address this, the linkers of subsequent bifunctional compounds were extended directly from the pyrrolidine ring nitrogen. A small library of hetero-bifunctional compounds with linker lengths several atoms shorter and longer than LC-1 were screened, and from this screen, LC-2 (also referred to herein as exemplary compound 90) was identified as a potent KRAS^{G12C}-degrading bifunctional compound (FIG. 2A). LC-2 covalently binds KRAS^{G12C} with a PTM or warhead, and recruits the E3 ligase VHL, inducing rapid and sustained KRAS^{G12C} degradation leading to suppression of MAPK signaling in both homozygous and heterozygous KRAS^{G12C} cell lines. LC-2 demonstrates that hetero-bifunctional compound mediated degradation is a viable option for attenuating oncogenic KRAS levels and downstream signaling in cancer cells. LC-2 induced maximal degradation of endogenous KRAS^{G12C} at concentrations as low as 2.5 μM with a D_{Max} of >75% and a DC₅₀ of 0.59±0.2 μM in NCI-H2030 cells (FIG. 2C). At 10 μM LC-2, a KRAS^{G12C} band running at the same molecular weight as LC-1-modified KRAS^{G12C} was observed. The emergence of this undegraded higher molecular weight band suggests the start of a “hook-effect” at high LC-2 concentrations. The “hook-effect” is a hallmark of bifunctional protein-degrading compounds, whereby at high drug concentrations, the formation of unproductive dimers with target or with E3 ligase outcompete formation of the ternary complex necessary for degradation (Buckley, D. L., et al., HaloPROTACS: Use of Small Molecule PROTACs to Induce Degradation of HaloTag Fusion Proteins. Acs Chem Biol 2015, 10 (8), 1831-7).

MRTX is known to be selective for mutant KRAS^{G12C} over other KRAS mutants (Hallin et al. 2020). To explore the specificity of LC-2, KRAS degradation was examined in HCT 116 cells, which harbor a heterozygous KRAS^{G13D} mutation. No engagement or degradation of KRAS^{G13D} was observed in the presence of LC-2 up to 10 μM (FIG. 3B). These data further suggest that LC-2 selectively engages and degrades mutant KRAS^{G12C} protein.

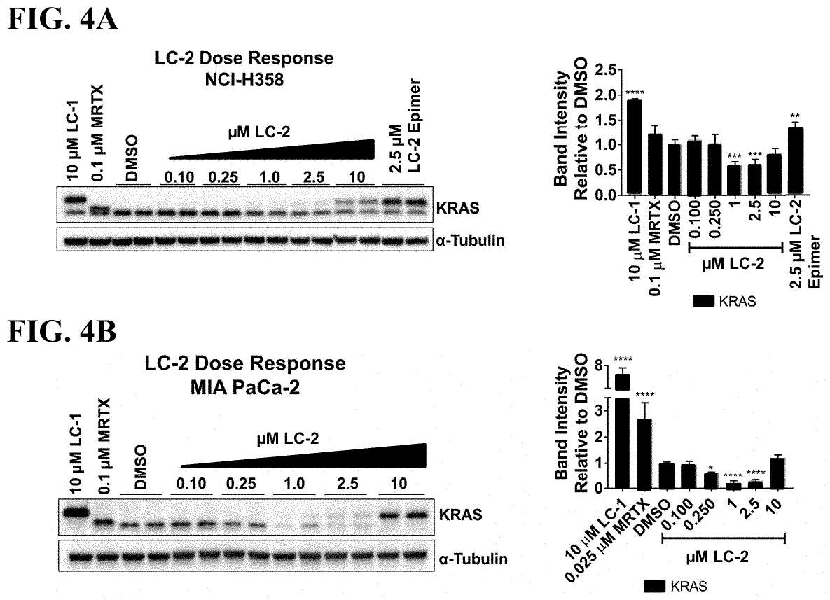

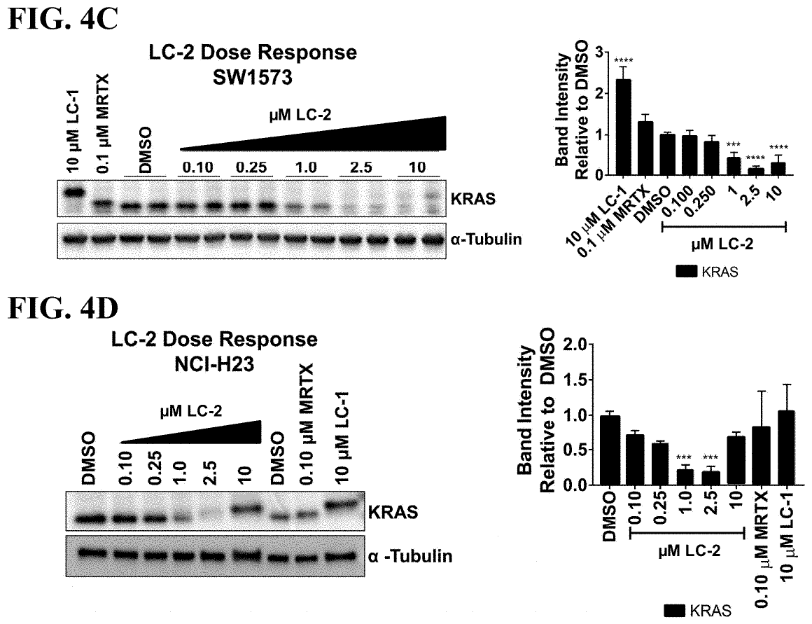

In addition, LC-2 in 5 different KRAS^{G12C} cell lines and observed DC₅₀ values between 0.25 and 0.76 μM as well as D_{Max} values ranging from >75-90% (Table 1). LC-2 can degrade mutant KRAS in both homozygous and heterozygous cell lines with varying sensitivities to MRTX (Hallin et al. 2020). Great than 50% degradation was observed in NCI-H23 cells, which are heterozygous. Theoretically, since these cells carry one wild type and one mutant KRAS^{G12}c allele, one would expect a maximum of 50% degradation if expression were equal, as we see for NCI-H358 cells (FIG. 4A). However, in siRNA knockdown experiments using KRAS^{G12C} specific siRNA, nearly complete loss of KRAS is observed for NCI-H23 cells, which is consistent with the degradation observe with LC-2 (Sunaga, N., et al., Knockdown of oncogenic KRAS in non-small cell lung cancers suppresses tumor growth and sensitizes tumor cells to targeted therapy. Mol Cancer Ther 2011, 10 (2), 336-46). Cumulatively, these data show that MRTX-based, VHL-recruiting bifunctional compounds can engage and degrade KRAS^{G12C} in multiple cancer cell lines.

TABLE 1
LC-2 Induces degradation of endogenous KRAS^{G12C} in multiple
KRAS mutant Cancer cell lines: bifunctional compound activity
in a panel of KRAS^{G12C} cancer cell lines

| | KRAS^{G12C} | | | Mirati |
|---|---|---|---|---|
| Cell Line | Genotype | DC₅₀ (μM) | Dₘₐₓ (%) | Sensitivity²¹ |
| NCI-H2030 | Homozygous | 0.59 ± 0.2 | >75 | + |
| MIA PaCa2 | Homozygous | 0.32 ± 0.08 | >75 | ++ |
| SW1573 | Homozygous | 0.76 ± 0.3 | >85 | − |
| NCI-H23 | Heterozygous | 0.25 ± 0.08 | >90 | +++ |
| NCI-H358 | Heterozygous | 0.52 ± 0.3 | >40 | +++ |

DC₅₀ at which 50% of the maximal degradation (Dₘₐₓ) is reached.

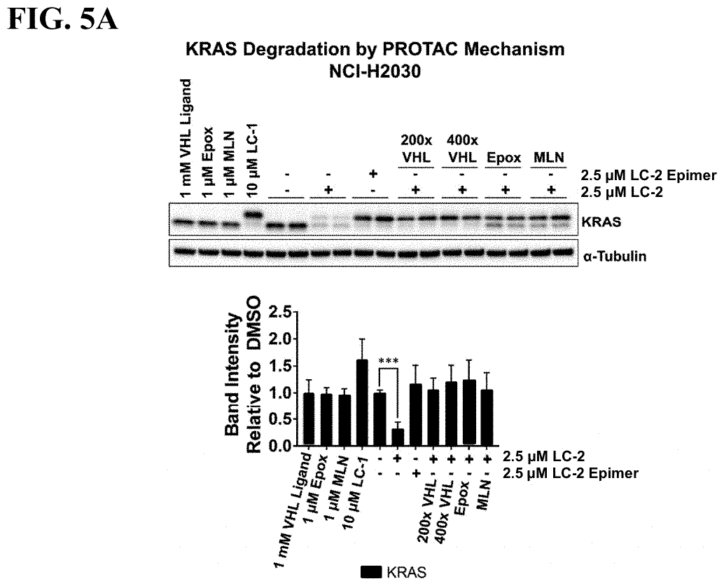

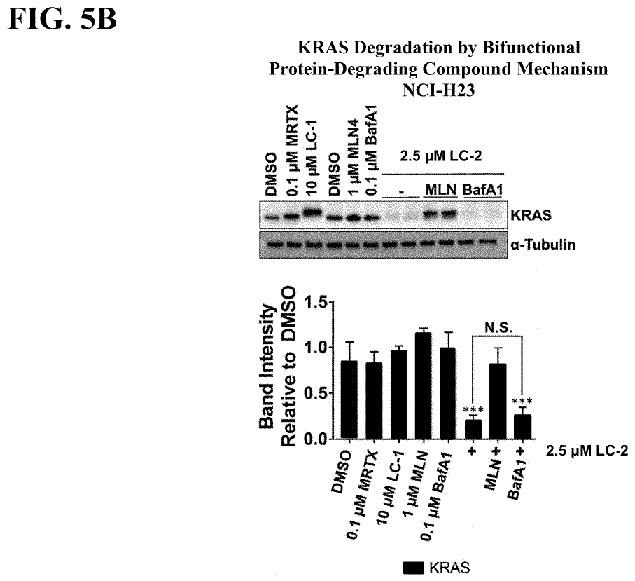

The hydroxy proline moiety of the VHL ligand confers binding to the E3 ligase, while inversion of the absolute stereochemistry of the 4-hydroxy proline moiety abrogates VHL binding (Buckley et al. 2012). Therefore, LC-2 Epimer (FIG. 2A) was synthesized as a physicochemically-matched negative control molecule that is unable to recruit VHL. When NCI-H2030 cells were treated with 2.5 μM LC-2 Epimer for 4 hours, only KRAS engagement was observed, whereas 2.5 μM LC-2 induced significant degradation (˜65%; FIG. 5A).

Bifunctional protein degrading compounds target proteins for degradation via the proteasome by facilitating their ubiquitination, which is dependent on the formation of a ternary complex (Sakamoto et al. 2001; Bondeson et al. 2015; Bondeson et al 2018) between the target protein, the bifunctional compound, and the E3 ligase—in this case, VHL. Since excess VHL ligand inhibits ternary complex formation, competition experiments were performed in NCI-H2030 cells that were pre-treated for 1 hour with molar excess of VHL ligand before being treated with 2.5 μM LC-2. Competition of LC-2 with VHL ligand rescued KRAS^{G12C} levels (FIG. 5A) by preventing bifunctional compound engagement with VHL. However, the higher molecular weight KRAS^{G12C} band observed upon LC-2 treatment demonstrates that the bifunctional compound was nevertheless still able to engage KRAS^{G12C}.

Neddylation of CUL2, a VHL adaptor protein, is necessary for proper assembly and function of the VHL E3 ligase complex (Merlet, J. et al., Regulation of cullin-RING E3 ubiquitin-ligases by neddylation and dimerization. Cell Mol Life Sci 2009, 66 (11-12), 1924-38). To further investigate whether LC-2 induced degradation of KRAS^{G12C} occurs via a bona fide bifunctional protein degrading compound mechanism, NCI-H2030 cells were treated with 1 μM of the neddylation inhibitor MLN924 or 1 μM of the proteasome inhibitor epoxomicin, before being treated with 2.5 μM LC-2 (Meng, L., et al., Epoxomicin, a potent and selective proteasome inhibitor, exhibits in vivo antiinflammatory activity. Proc Natl Acad Sci USA 1999, 96 (18), 10403-8; Soucy, T. A., et al., An inhibitor of NEDD8-activating enzyme as a new approach to treat cancer. Nature 2009, 458 (7239), 732-U67). Both inhibitors rescued KRAS^{G12C} levels suggesting KRAS^{G12C} degradation by LC-2 is both proteasome- and neddylation-dependent (FIG. 5A).

KRAS is tethered to the plasma membrane, and it is possible that monoubiquitination of KRAS^{G12C} could induce endocytosis and degradation of KRAS^{G12C} through the lysosomal pathway (Lu, A., et al., A clathrin-dependent pathway leads to KRas signaling on late endosomes en route to lysosomes. J Cell Biol 2009, 184 (6), 863-879)⁴⁵. Therefore, we also tested whether bafilomycin A1 (BafA1), an inhibitor of lysosomal acidification, could rescue KRAS^{G12C} degradation (Oda, K., et al., Bafilomycin-A1 Inhibits the Targeting of Lysosomal Acid-Hydrolases in Cultured-Hepatocytes. Biochem Bioph Res Co 1991, 178 (1), 369-377). Pre-treatment of NCI-H23 with BafA1 was unable to rescue LC-2 induced KRAS^{G12C} degradation, whereas neddylation inhibition again rescued KRAS degradation (FIG. 5B). Taken together these data show that LC-2-induced KRAS^{G12C} degradation is dependent on ternary complex formation with VHL and a functioning ubiquitin proteasome system, but not dependent on the lysosome.

LC-2 Induce Rapid and Sustained KRAS^{G12C} Degradation in Multiple Cancer Cell Lines.

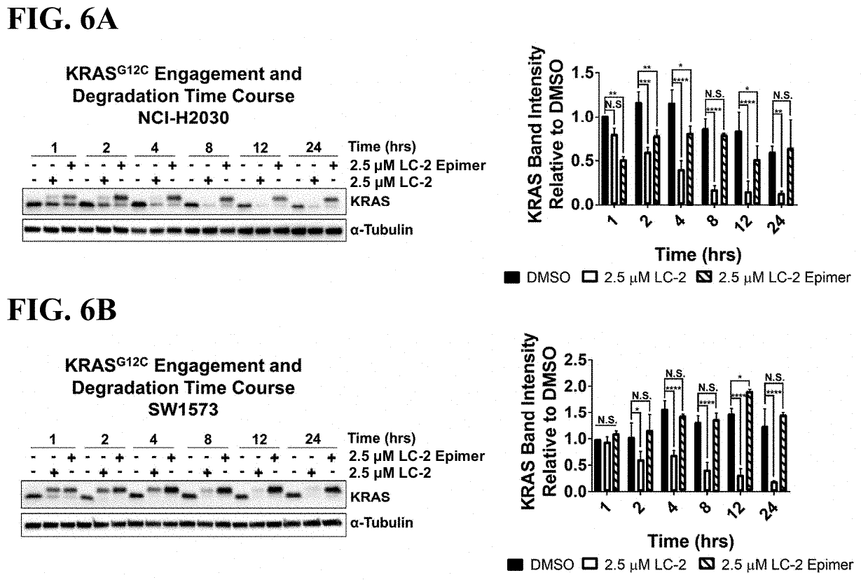

To explore hetero-bifunctional protein degrading compound induced KRAS^{G12C} degradation kinetics, time course experiments were performed in NCI-H2030 cells and SW1573 cells using 2.5 μM LC-2 as the fixed concentration since it induced maximal degradation in all cell lines within 24 hours (FIG. 2A and FIG. 4A-4D). To distinguish between rates of target engagement and degradation, LC-2 Epimer was used as a negative control to monitor KRAS^{G12C} engagement. Quantitation of engagement was achieved by comparing the intensity of just the LC-2 Epimer modified band to the intensity of unbound KRAS in DMSO treated samples. For NCI-H2030 cells, KRAS^{G12C} binding was seen as early as 1 hour for both LC-2 and LC-2 Epimer (FIG. 6A). Maximal engagement and significant degradation occurred within 4 hours. Maximum degradation was reached by 8 hours in NCI-H2030 cells and persisted up to 24 hours. SW1573 cells showed faster kinetics with near maximal engagement at 1 hour. However, the degradation rate was slower than NCI-H2030 cells as maximal degradation was not observed until 12 hours (FIG. 6B).

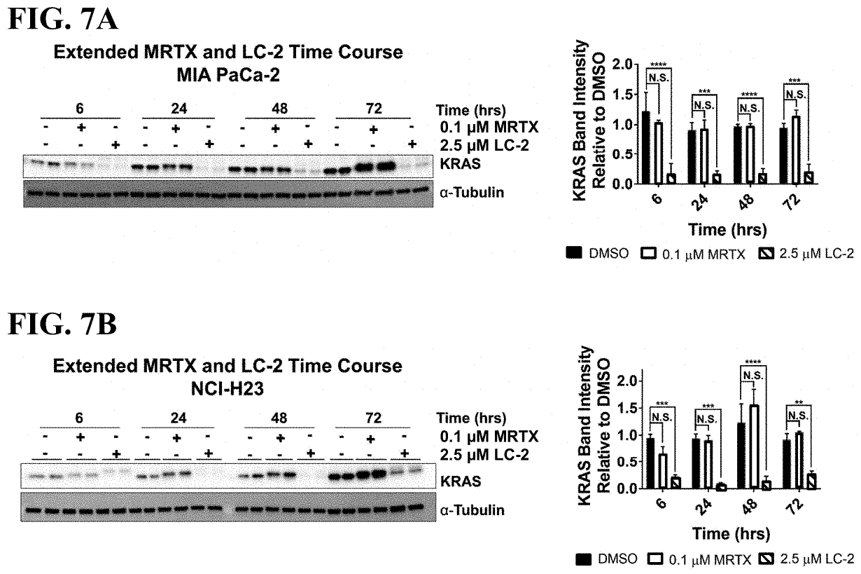

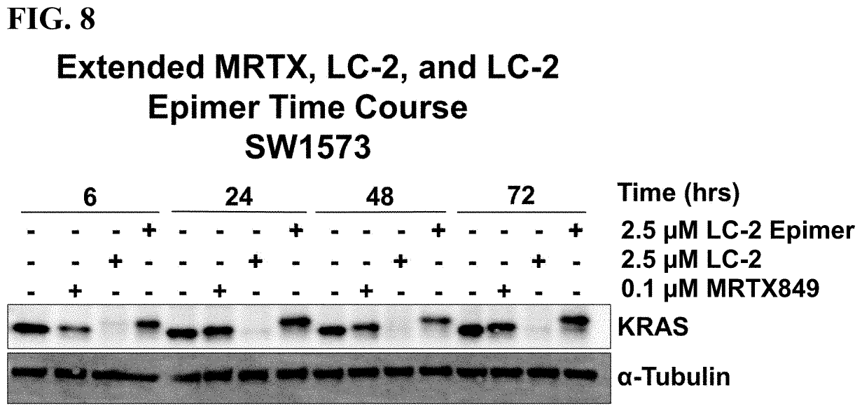

During our bifunctional protein degrading compound screen, it was observed that 0.1 μM of MRTX and 10 μM of LC-1 increased KRAS protein levels (FIG. 2C). Although Hallin, et al. did not observe increased KRAS^{G12C} protein levels with MRTX, our data is consistent with previous observations made with the KRAS^{G12C} inhibitor ARS1620 (Janes et al. 2018; Hallin et al. 2020). Therefore, it was examined how longer treatments with LC-2 would affect KRAS^{G12C} levels. MIA PaCa-2, NCI-H23, and SW1573 cells were treated with 2.5 μM of LC-2 for 6, 24, 48, and 72 hours. In all three cell lines, maximal KRAS degradation occurred within 24 hours and was sustained up to 72 hours (FIGS. 7A, 7B, and 8). LC-2 Epimer fully engaged KRAS^{G12C} in SW1573 cells, but did not decrease protein levels as expected (FIG. 8). In NCI-H23 cells, KRAS^{G12C} began to rebound at 72 hours. Taken together these data show that LC-2 is capable of rapid and sustained KRAS^{G12C} degradation in both homozygous and heterozygous cell lines. The ability to overcome increased KRAS^{G12C} expression suggests that degradation could be more beneficial than inhibition for prolonged attenuation of downstream signaling as has been observed previously with BRD4 degraders (Lu, J., et al., Hijacking the E3 Ubiquitin Ligase Cereblon to Efficiently Target BRD4. Chem Biol 2015, 22 (6), 755-63).

LC-2 Induced KRAS^{G12C} Degradation Modulates Erk Signaling in Homozygous and Heterozygous KRAS Mutant Cell Lines.

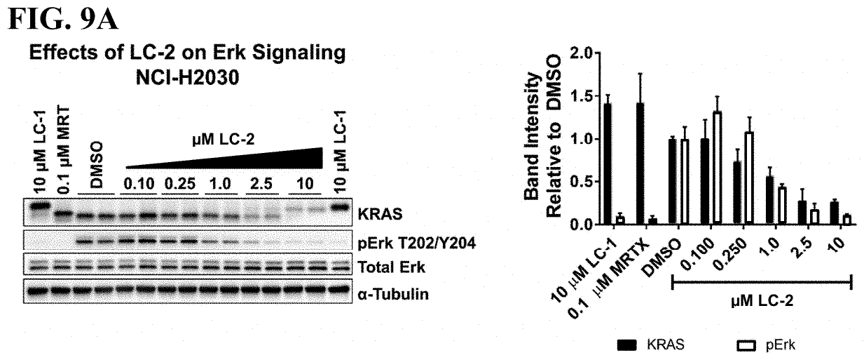

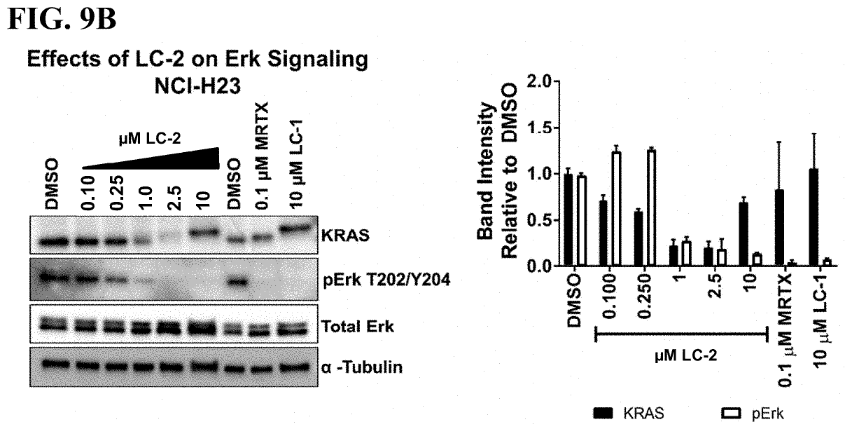

The ability of LC-2 to modulate Erk signaling was investigated in NCI-H2030 and NCI-H23 cells during a 24 hour dose response. In NCI-H2030 cells, pErk was detected and a dose-dependent decrease in signaling was observed (FIG. 9A). In addition, total Erk levels were elevated in a dose-dependent manner. NCI-H23 cells showed a similar dose-dependent decrease in pErk (FIG. 9B). Additionally, total Erk levels were elevated in a dose-dependent manner.

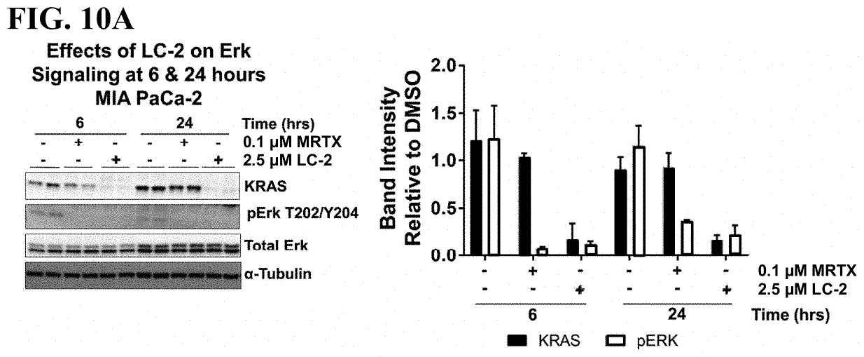

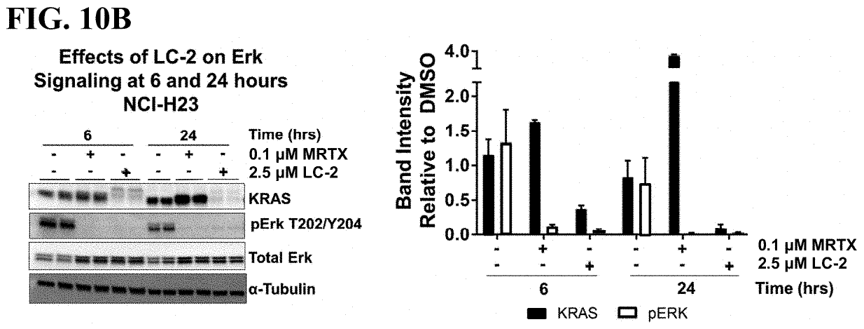

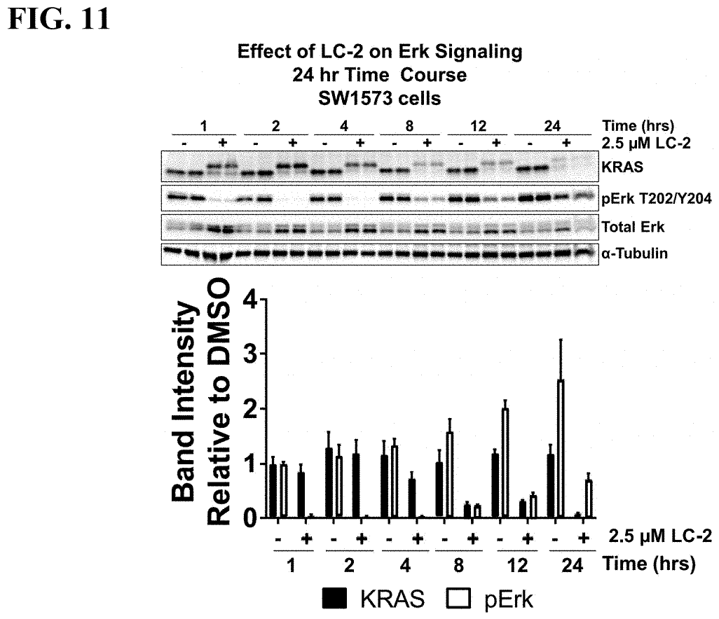

Signaling kinetics were monitored during a 24 hour time course in MIA PaCa2, NCI-H23, and SW1573 cells treated with 2.5 μM LC-2. Modulation of Erk signaling by both MRTX and LC-2 occurs within 6 hours in MIA PaCa-2 and NCI-H23 cells (FIGS. 10A, 10B, and 11). pErk was suppressed by both compounds at 6 and 24 hours in each cell lines. In SW1573 cells, phosphorylated Erk was inhibited by 2.5 μM LC-2 between 1 and 4 hours, however pErk levels rebounded between 8 and 24 hours (FIG. 11). Nonetheless, pErk levels were still significantly lower in LC-2 treated cells than DMSO treated cells at 24 hours. Total Erk was increased in LC-2 treated cells compared to DMSO at all time points indicating the initiation of a positive feedback loop upon KRAS^{G12C} degradation and pErk inhibition. Taken together, these data show that LC-2-induced KRAS^{G12C} degradation is capable of modulating downstream signaling and that differences in signaling between inhibition and degradation are cell line dependent.

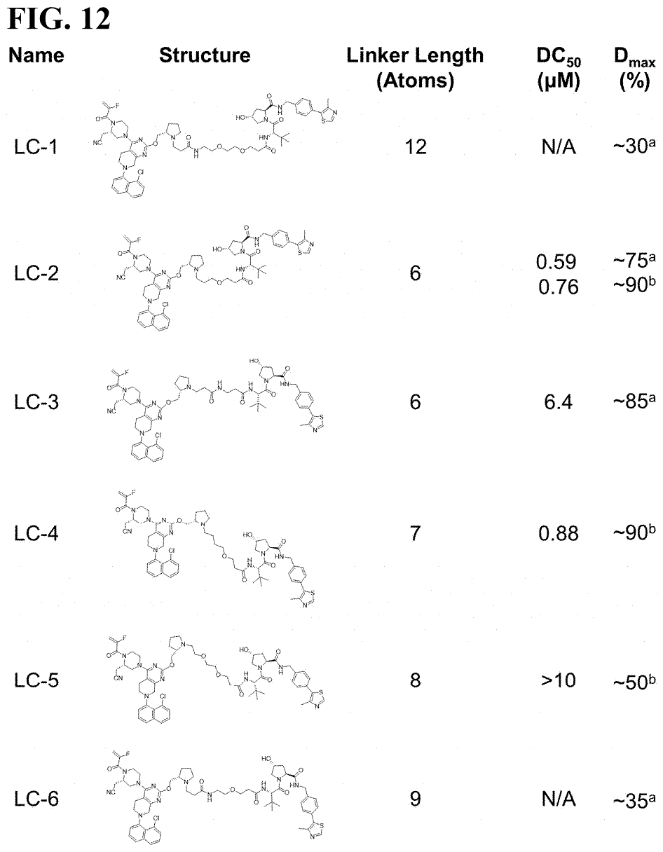

FIG. 12 presents the linker length (from the carbon adjacent to the pyrrolidine nitrogen to the carbon adjacent to the VHL carbonyl) and activity for each of exemplary compounds LC-1, LC-2, LC-3 (also referred to herein as exemplary compound 91), LC-4 (also referred to herein as exemplary compound 92), LC-5 (also referred to herein as exemplary compound 93), and LC-6 (also referred to herein as exemplary compound 94). Shorter linker lengths induce higher levels of degradation. ^{a}Data from NCI-H2030 cells. ^{b}Data from SW1573 cells.

TABLE 2
Two-way ANOVA analysis of FIG. 9A.
Effects of LC-2 on Erk Signaling
NCI-H2030

| | | 10 μM | 100 nM | 0.1 μM | 0.25 μM | 1.0 μM | 2.5 μM | 10 μM |
|---|---|---|---|---|---|---|---|---|
| | DMSO | LC-1 | MRTX849 | LC-2 | LC-2 | LC-2 | LC-2 | LC-2 |
| KRAS | N/A | * | * | N.S. | N.S. | ** | **** | **** |
| pERK | N/A | **** | **** | * | N.S. | **** | **** | **** |

Not Significant (N.S.);
* p < 0.05;
p < 0.001.

TABLE 3
Two-way ANOVA analysis of FIG. 9B.
Effects of LC-2 on Erk Signaling
NCI-H23

| | | 0.1 μM | 0.25 μM | 1.0 μM | 2.5 μM | 10 μM | 100 nM | 10 μM |
|---|---|---|---|---|---|---|---|---|
| | DMSO | LC-1 | LC-2 | LC-2 | LC-2 | LC-2 | MRTX849 | LC-1 |
| KRAS | N/A | N.S. | * | **** | ***** | N.S. | N.S. | N.S. |
| pERK | N/A | N.S. | N.S. | N.S. | * | N.S. | * | * |

Not Significant (N.S.);
* p < 0.05;
** p < 0.01.

TABLE 4
Two-way ANOVA analysis of FIG. 10A.
Effects of LC-2 on Erk Signaling at 6 and 24 hours
MIA PaCa-2

| | Time (hours) | | | | | |
|---|---|---|---|---|---|---|
| | 6 | | | 24 | | |
| | | 100 nM | 2.5 μM | | 100 nM | 2.5 μM |
| | DMSO | MRTX849 | LC-2 | DMSO | MRTX849 | LC-2 |
| KRAS | N/A | N.S. | ** | N.S. | N.S. | ** |
| pERK | N/A | **** | ** | N.S. | * | ** |

Not Significant (N.S.);
* p < 0.05;
** p < 0.01;
**** p < 0.001.

TABLE 5
Two-way ANOVA analysis of FIG. 10B.
Effects of LC-2 on KRAS Signaling at 6 and 24 hours
NCI-H23

| | Time (hours) | | | | | |
|---|---|---|---|---|---|---|
| | 6 | | | 24 | | |
| | | 100 nM | 2.5 μM | | 100 nM | 2.5 μM |
| | DMSO | MRTX849 | LC-2 | DMSO | MRTX849 | LC-2 |
| KRAS | N/A | N.S. | ** | N.S. | **** | **** |
| pERK | N/A | **** | **** | * | **** | **** |

Not Significant (N.S.);
* p < 0.05;
** p < 0.01;
**** p < 0.001.

TABLE 6
Two-way ANOVA analysis of FIG. 11.
Effects of LC-2 on KRAS Signaling 24 Hour Time Course
SW1573 Cells

| | Time (hours) | | | | | | | | | | | |
|---|---|---|---|---|---|---|---|---|---|---|---|---|
| | 1 | | 2 | | 4 | | 8 | | 12 | | 24 | |
| | | 2.5 μM | | 2.5 μM | | 2.5 μM | | 2.5 μM | | 2.5 μM | | 2.5 μM |
| | DMSO | LC-2 | DMSO | LC-2 | DMSO | LC-2 | DMSO | LC-2 | DMSO | LC-2 | DMSO | LC-2 |
| KRAS | N/A | N.S. | N.S. | N.S. | N.S. | N.S. | N.S. | ** | N.S. | ** | N.S. | *** |
| pERK | N/A | **** | N.S. | **** | N.S. | **** | * | ** | **** | * | **** | N.S. |

Not Significant (N.S.);
* p < 0.05;
** p < 0.01;
*** p < 0.005;
p < 0.001.

LC-2 couples the covalent KRAS^{G12C} inhibitor MRTX to the VHL ligand. VHL recruitment to KRAS^{G12C} induces endogenous KRAS ubiquitination and degradation with DC₅₀ values ranging from 0.25 to 0.76 μM. Tapid engagement, sustained KRAS degradation, and attenuated pErk signaling for up to 72 hours was observed in several KRAS^{G12C} mutant cell lines. This compound will facilitate further exploration of how KRAS degradation influences downstream signaling and the viability of KRAS^{G12C} mutant cancer cells with more precise temporal control than nucleic acid-based knockdown methods.

Further studies will focus on understanding the importance of the KRAS^{G12C} ligand, the recruited E3 ligase, or combination of the two factors in imparting LC-2's activity. Conducting ternary complex assays by SPR and/or monitoring the ability of these compounds to induce ubiquitination by using tandem ubiquitin binding entity (TUBE) pulldowns followed by immunoblotting could address these questions (Hjerpe, R., et al., Efficient protection and isolation of ubiquitylated proteins using tandem ubiquitin-binding entities. Embo Rep 2009, 10 (11), 1250-1258).

It will be interesting to determine whether degradation alone can overcome Erk signaling reactivation and/or if combination of KRAS degradation with RTK inhibition could further enhance antiproliferative effects. In addition to the re-wiring of sensitive cells, there are known cell lines, such as SW1573 (used in this work) and NCI-H1792, that are inherently resistant to the anti-proliferative effects of KRAS^{G12C} inhibition. Recently, it was shown that siRNA mediated knockdown in these cells, but not KRAS^{G12C} inhibition, resulted in ˜50% decreased cell viability (Misale et al. 2019). Therefore, it will be interesting to determine if KRAS^{G12C}-induced degradation of KRAS^{G12C} by LC-2 is also similarly antiproliferative in these cell lines.

Materials and Methods:

Cell Lines and Reagents:

NCI-H2030 (CRL-5914), MIA PaCa-2 (CRL-1420), NCI-H23 (CRL-5800), NCI-H358 (CRL-5807), and HCT-116 (CCL-247) cells were obtained from ATCC, expanded immediately, and frozen down. Vials were thawed and used within 20 passages. SW1573 cells were a gift from Arvinas and were handled in the same manner. NCI-H2030, NCI-H23, and NCI-H358 were cultured in RPMI (ATCC30-2001) supplemented with 10% FBS (Biological Industries; cat. no. S1480) and 1% Penicillin-Streptomycin (ThermoFisher; cat. no. 15140122). HCT-116 cells were maintained in high glucose DMEM (ThermoFisher; cat. no. 11965084) supplemented as above. MIA PaCa-2 cells were cultured in high glucose DMEM supplemented with 10% FBS, 2.5% horse serum (ThermoFisher; cat. no. 26050-088; lot 2109875), and 1% Penicillin-Streptomycin. SW1573 cells were maintained in DMEM/F12 Nutrient Mix with GlutaMAX supplement (ThermoFisher; cat. no. 10565018) with 10% FBS and 1% Penicillin-Streptomycin added. DPBS (ThermoFisher; cat. no. 14190250) was used to wash cells and 0.25% Trypsin-EDTA (ThermoFisher; cat. no. 252000-056) was used to detach cells for passaging. MRTX849 was purchased from ChemieTek (cat. no. CT-MRTX849), epoxomicin was purchased from Astatech (cat. no. 41576), MLN924 (pevonedistat) was purchased from Selleckchem (cat. no. S7109), and bafilomycin A1 was purchased from Millipore Sigma (cat. no. B1793). The VHL ligand was generously provided by Arvinas.

Time Course Assays:

Between 2.5×10⁵ and 6.0×10⁵ cells were seeded into 6-well plates (Corning; cat. no. 353046). The next day, media was removed and cells were treated with 2.5 μM LC-2 for 1, 2, 4, 8, 12, or 24 hours. Cells were treated with 100 nM MRTX849 and 2.5 μM LC-2 for 6, 24, 48, or 72 hours for longer time course experiments. For 24-hour time course experiments, cells were treated at the indicated times and concurrently lysed in RIPA buffer supplemented as described previously. For longer time course experiments and time courses in which cells were treated with LC-2 or LC-2 Epimer, cells were concurrently treated with either compound, then lysed by scraping in RIPA buffer at the indicated time points.

Competition, Proteasome Inhibition, and Neddylation Inhibition Experiments

Between 2.5×10⁵ and 5.0×10⁵ cells were seeded into 6-well plates. The next day cells were pretreated with DMSO, 500 μM or 1 mM VHL ligand, 1 μM epoxomicin, 1 μM MLN924, or 100 nM M bafilomycin A1 for 1 hour. Media was then removed and cells were treated with DMSO, 2.5 μM LC-2 plus DMSO, 2.5 μM LC-2 Epimer plus DMSO, or co-treated with 2.5 μM LC-2 and the corresponding competitor/inhibitor. H2030 cells were treated for 4 hours and H23 cells were treated for 24 hours, after which cells were lysed by scraping in RIPA buffer supplemented as described previously.

Immunoblotting:

Cell lysates were clarified at 21000×g for 15 mins at 4° C. Protein levels were quantified using a 50:1 mixture of bicinchoninic acid solution (Millipore Sigma; cat. no. B9643) and 4% (w/v) copper(II) sulfate solution (Millipore Sigma; cat. no. C2284) incubated at 37° C. for 30 mins. Absorbance values at 560 nm were read on an EnVision 2101 Multilabel Reader (PerkinElmer). Proteins were separated using 26 well Criterion TGX precast 4-15% (cat. no. 5671085) or 8-16% (cat. no. 5671105) gradient midi gels. After separation, proteins were transferred to nitrocellulose or PVDF membranes at 76 V for 2 hours at 4° C. Blots were then blocked in 5% milk in tris-buffered saline with Tween-20 (TBST; 20 mM Tris, 150 mM NaCl, 0.02% Tween-20). After blocking, blots were incubated in primary antibody overnight (12-18 hours) at 4° C. or for 2 hours at room temperature with mild agitation at the manufacturer's indicated dilution in either 5% milk or 5% BSA in TBST. Blots were then washed thrice with TBST for 5 mins at room temperature. After washing, blots were incubated with 1:5,000-1:10,000 of donkey anti-rabbit (GE Life Sciences; NA934) or sheep anti-mouse (GE Life Sciences; NA931) secondary antibody diluted in 5% milk for 1 hour at room temperature with mild agitation. Blots were again washed thrice with TBST for 5 mins. Chemiluminescent signal was generated using Amersham ECL Prime Western Blotting Detection Reagant (GE Life Sciences; cat. no. RPN2232) or SuperSignal West Femto Maximum Sensitivity Substrate (ThermoFisher; cat. no. 34095). Images were obtained using a Bio-Rad ChemiDoc Imager. Fluorescent α-tubulin images were collected with the Bio-Rad ChemiDoc Imager using an Alexa488 filter. The primary antibodies used in this work include: KRAS (LSBio; clone 2C1; cat. no. LS-C175665), p-p42/44 MAPK (phospho T202/Y204) (pErk; Cell Signaling Technologies (CST); cat. no. 4370S or 9106S), p42/44 MAPK (Erk1/2; CST; cat. no. 4695S), alpha-tubulin (CST; 2144S), and alpha-tubulin w/AlexaFluor488 (Millipore; cat. no. 16-232).

Quantification and Statistical Analysis:

Band intensities were quantified using BioRad's Image Lab Software. Total KRAS levels were examined by quantifying levels of both conjugated KRAS^{G12C} and unbound KRAS (wt or mutant) using an analysis box that spanned the two bands. This same sized box was used to quantify unbound KRAS in DMSO treated samples to account for background, except for LC-2 Epimer in FIGS. 6A and 6B. To quantify LC-2 Epimer engagement only the top, bifunctional compound bound, band was quantified and band intensity was normalized to unbound KRAS in DMSO treated samples using a similar sized analysis box. Data was analyzed by computing one-way ANOVAs or two-way ANOVAs (for grouped data) using multiple comparisons in which the mean of DMSO was compared to all treatment means using GraphPad Prism 7. For time courses, protein levels were compared to DMSO for each time point. DC₅₀ and D_{Max} were quantified by fitting data to an inhibitor vs. dose response non-linear regression using GraphPad Prism 7.

Chemical Synthesis:

General Considerations.

Chemicals used for synthesis were purchased from commercial sources and were used without further purification. Flash chromatography was performed using Biotage flash chromatography system using pre-packed columns. ¹H NMR and ¹³C NMR spectra were recorded on an Agilent DD2 600 NMR spectrometer (600 MHz for ¹H and 151 MHz for ¹³C). The values of chemical shifts (δ) are reported in p.p.m. Coupling constants (J) are reported in Hz. High-resolution mass spectra (HRMS) were recorded on a Waters Xevo QTOF LCMS with ESI using a Waters Acquity UPLC. HPLC purifications were performed on a reverse-phase column using a Gilson HPLC system.

Assay for Testing KRas Degradation Driven by Compounds of Tables 8A and 8B, Including Exemplary Hetero-Bifunctional Compounds Designed to Target KRas

Reagents. SW1573 (CRL-2170) and MiaPaCa-2 (CRL-1420) cells were purchased from ATCC. Both cell lines are homozygous for the G12C mutation in KRAS. SW1573 cells were cultured in DMEM+F12+Glutamax (+Sodium Bicarbonate, +sodium pyruvate, Thermofisher scientific, #10565) supplemented with 10% FBS (Thermofisher scientific, #2060357) and 1% Antibiotic-Antimycotic (Thermofisher scientific, #15240096). MiaPaCa-2 cells were cultured in DMEM+Glutamax media (Thermofisher scientific, #10566) supplemented with 10% FBS (Thermofisher scientific, #2060357) and 1% Antibiotic-Antimycotic (Thermofisher scientific, #15240096). For degradation assays, 384 well plates were purchased from Corning (Corning® CellBIND® 384-well Flat Clear Bottom Black Polystyrene Microplates, Low Flange, #3770). Gambogic acid was purchased from Selleckchem (#S2448). Alamar blue was purchased from Thermofisher scientific (A50101) and used according to manufacturer instructions. The Nano-Glo® HiBit Lytic Detection System and Nano-Glo® HiBit Blotting System were purchased from Promega (#N3050 and #N2410) and used according to manufacturer instructions.

HiBit-Tagged Cell Line Generation.

To create the SW1573 HiBit-KRAS^{G12C} cell line, the nucleotide sequence encoding the 11 amino acid HiBit tag from Promega (Schwinn, M. K. et al. (2018) CRISPR-mediated tagging of endogenous proteins with a luminescent peptide. ACS Chem. Biol. 13(2), 467-474) was appended to the N-terminus of the KRAS^{G12C} coding sequence. This synthesized DNA was cloned into a modified pcDNA3.1 vector under the control of the thymidine kinase (TK) promoter. This HiBit-KRAS^{G12C} expressing plasmid was transfected into parental SW1573 cells and colonies were selected using 400 μg/mL zeocin. The resulting clonal lines were screened for expression of HiBit-KRAS^{G12C} protein using Nano-Glo® HiBit Blotting System. A luminescent band of the correct size for HiBit-KRAS^{G12C} was detected in several clones. Clone E6 was selected for further studies.

To create the MiaPaCa-2 HiBit-KRAS^{G12C} cell line, CRISPR-Cas9 was used to insert the HiBit tag onto the N-terminus of one or more of the four endogenous KRAS^{G12C} alleles. After knock-in of the tag, individual clones were isolated by single cell dilution and screened for HiBit insertion by PCR. Clones containing the HiBit tag were sequenced and one was identified that contained the desired HiBit-KRAS^{G12C} sequence at one allele while the remaining three KRAS^{G12C} alleles were unaffected. This line was used for all subsequent studies.

Degradation Assays.

Either SW1573 HiBit-KRas^{G12C} or MiaPaCa-2 HiBit-KRas^{G12C} cells were plated at 9000 cells/well in a 384 well plate (Corning #3770). Exemplary heterobifunctional molecules and positive controls were diluted in the appropriate cell media and applied to the plated cells resulting in a final concentration titration of 10 μM to 20 nM in either 0.1% or 0.5% dimethyl sulfoxide (DMSO). Gambogic acid was used as a positive control for cytotoxicity and titrated from 10 μM to 78 nM. All assays were performed in triplicate. Cells were treated with compounds for 24 hours at 37° C. in an incubator containing 5% CO₂. Following treatment, alamar blue was used to determine if any compounds led to a loss in cell viability after 24 hours. The 10× alamar blue reagent was added to a final concentration of 1× as recommended by the manufacturer and cells were returned to the incubator for 4 hours. Plates were then allowed to equilibrate to room temperature and fluorescence was measured using a Perkin-Elmer EnVision. After completion of the fluorescence read, the liquid from each well was removed and the cells were washed with PBS. The Nano-Glo® HiBiT detection reagent was prepared according to the manufacturer's instructions and added to each well after the PBS wash was removed. Following incubation at room temperature for 45 minutes, the luminescence was read using the ultra-sensitive luminescence aperture on the Envision instrument.

Data Analysis.

Fluorescence values from the alamar blue treatment were normalized to the DMSO only control for each compound titration. We hypothesized that compounds that led to a ≥50% loss in cell viability of the KRAS-independent SW1573 HiBit-KRas^{G12C} cells were likely generally cytotoxic and loss of HiBit-KRAS^{G12C} signal from these compounds may be due to lower cell number and not specific KRAS^{G12C} degradation. These points were therefore excluded from the data analysis for degradation. HiBit luminescence was normalized to the DMSO only control and the fractional HiBit signal was plotted versus the log of the exemplary compound concentration and fit to a 4-parameter dose-response model to obtain the concentration of exemplary compound that leads to half maximal degradation (DC₅₀) as well as the maximum degradation observed (Dₘₐₓ, conventionally expressed as a percentage of control), which are below in Table 8A and 8B for the exemplary compounds of Table 7.

TABLE 7
Exemplary bifunctional compounds of the present disclosure

| Ex. No. | Chemical Structure | IUPAC Name |
|---|---|---|
| 1 | 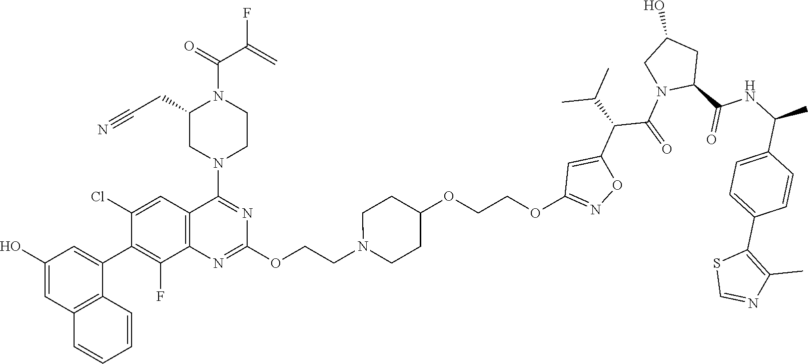 [SMILES: C=C(F)C(=O)N1CCN(c2nc(OCCN3CCC(OCCOc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-{3-[2-({1- [2-({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}oxy)ethoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine-2- carboxamide |
| 2 | 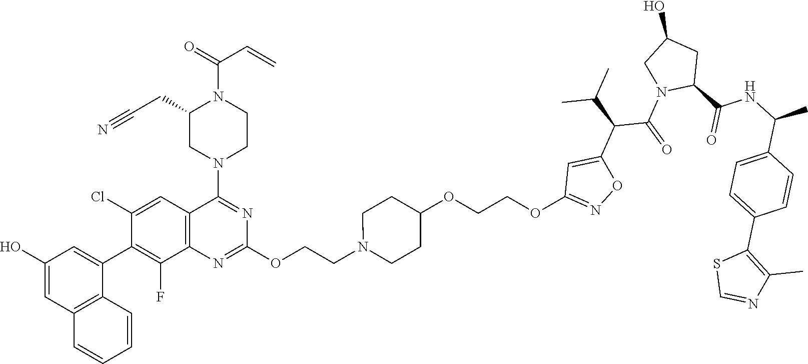 [SMILES: C=CC(=O)N1CCN(c2nc(OCCN3CCC(OCCOc4cc([C@H](C(=O)N5C[C@@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4S)-1-[(2R)-2-{3-[2-({1- [2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}oxy)ethoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine-2- carboxamide |
| 3 | 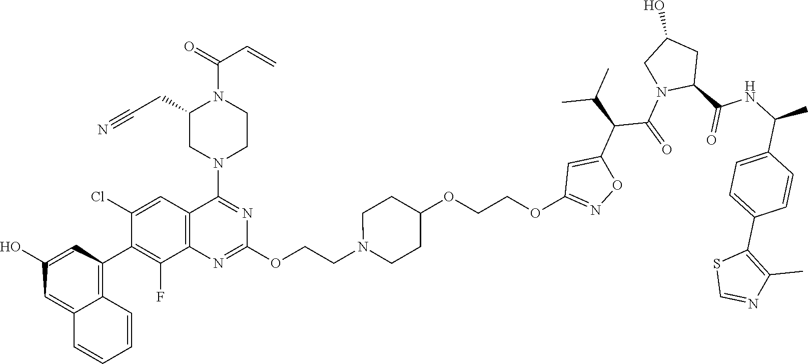 | (2S,4R)-1-((R)-2-(3-(2-((1-(2- (((S)-4-((S)-4-acryloyl-3- (cyanomethyl)piperazin-1-yl)- 6-chloro-8-fluoro-7-(3- hydroxynaphthalen-1- yl)quinazolin-2- yl)oxy)ethyl)piperidin-4- yl)oxy)ethoxy)isoxazol-5-yl)- 3-methylbutanoyl)-4-hydroxy- N-((S)-1-(4-(4-methylthiazol- 5-yl)phenyl)ethyl)pyrrolidine- 2-carboxamide |
| 4 | 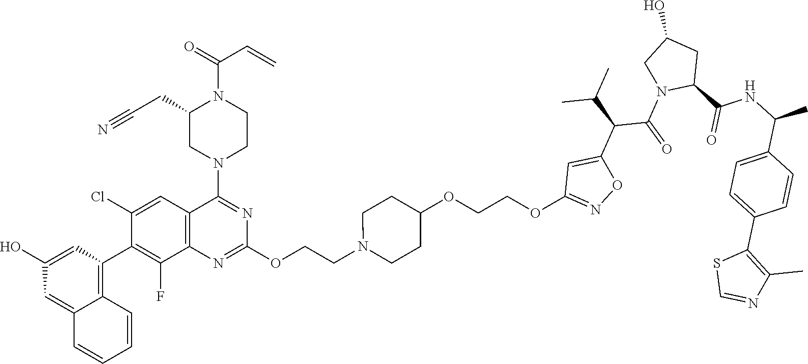 | (2S,4R)-1-((R)-2-(3-(2-((1-(2- (((R)-4-((S)-4-acryloyl-3- (cyanomethyl)piperazin-1-yl)- 6-chloro-8-fluoro-7-(3- hydroxynaphthalen-1- yl)quinazolin-2- yl)oxy)ethyl)piperidin-4- yl)oxy)ethoxy)isoxazol-5-yl)- 3-methylbutanoyl)-4-hydroxy- N-((S)-1-(4-(4-methylthiazol- 5-yl)phenyl)ethyl)pyrrolidine- 2-carboxamide |
| 5 | 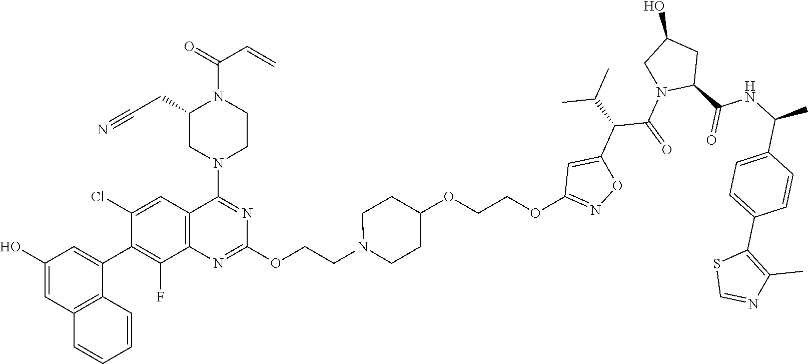 [SMILES: C=CC(=O)N1CCN(c2nc(OCCN3CCC(OCCOc4cc([C@@H](C(=O)N5C[C@@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4S)-1-[(2S)-2-{3-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}oxy)ethoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine-2- carboxamide |
| 6 | 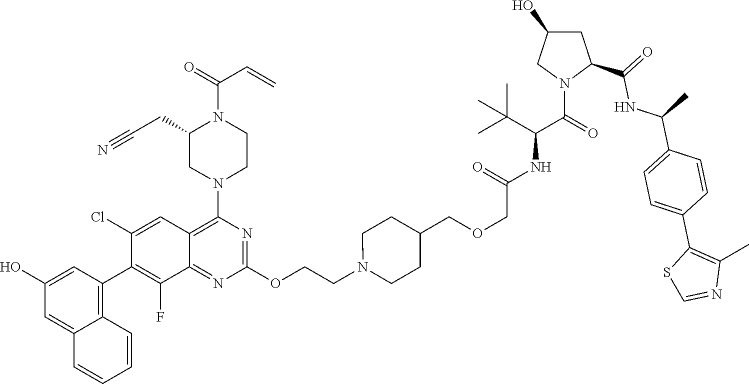 [SMILES: C=CC(=O)N1CCN(c2nc(OCCN3CCC(COCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4S)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 7 | 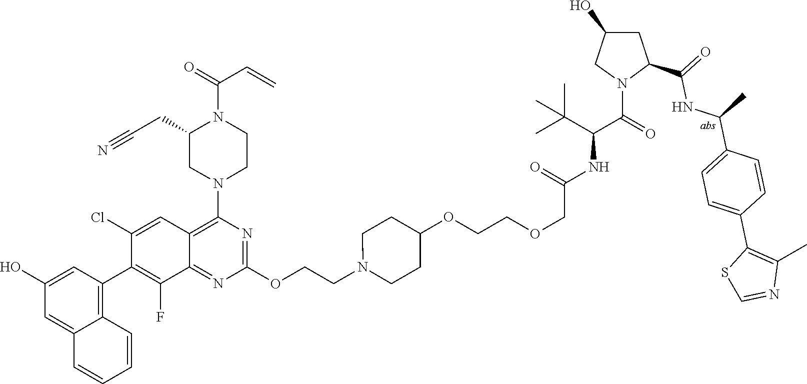 [SMILES: C=CC(=O)N1CCN(c2nc(OCCN3CCC(OCCOCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4S)-1-[(2S)-2-{2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 8 | 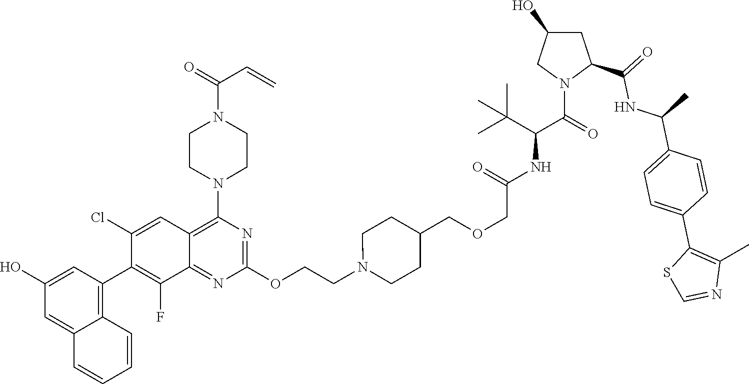 [SMILES: C=CC(=O)N1CCN(c2nc(OCCN3CCC(COCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4S)-1-[(2S)-2-(2-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 9 | 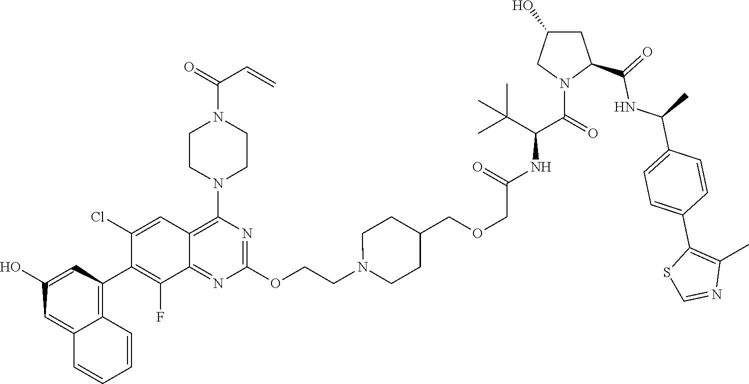 | (2S,4R)-1-[(2S)-2-(2-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 10 | 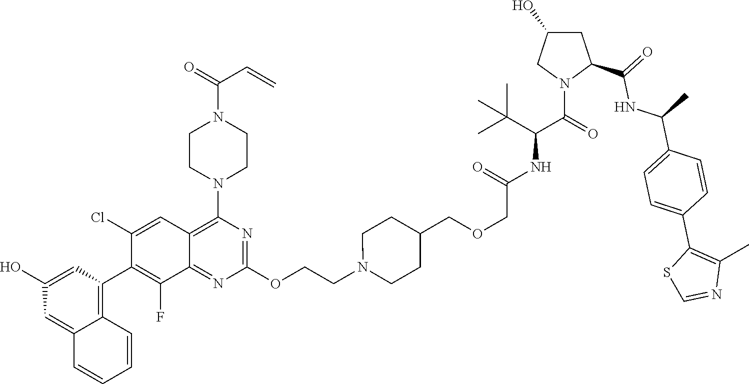 | (2S,4R)-1-[(2S)-2-(2-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 11 | 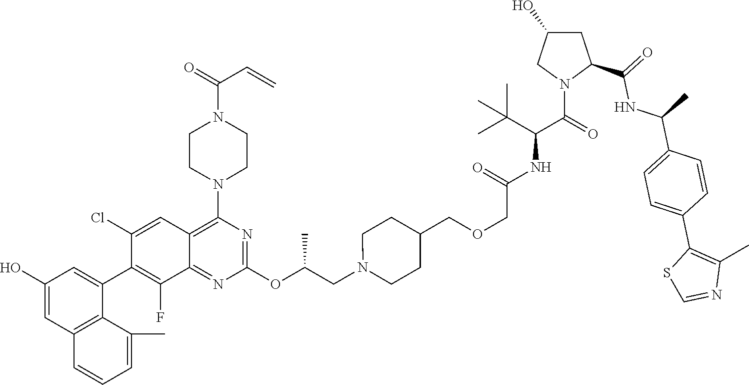 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5cccc(C)c45)c(Cl)cc23)CC1] | (2S,4R)-1-[(2S)-2-[2-({1- [(2R)-2-{[6-chloro-8-fluoro-7- (3-hydroxy-8- methylnaphthalen-1-yl)-4-[4- (prop-2-enoyl)piperazin-1- yl]quinazolin-2- yl]oxy}propyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 12 | 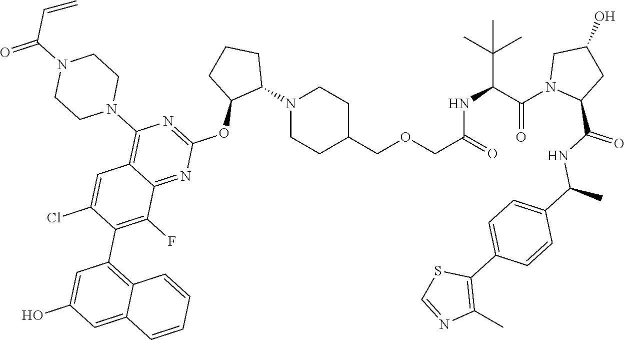 [SMILES: C=CC(=O)N1CCN(c2nc(O[C@H]3CCC[C@@H]3N3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-({1- [(1S,2S)-2-{[6-chloro-8-fluoro- 7-(3-hydroxynaphthalen-1-yl)- 4-[4-(prop-2-enoyl)piperazin- 1-yl]quinazolin-2- yl]oxy}cyclopentyl]piperidin- 4-yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 13 | 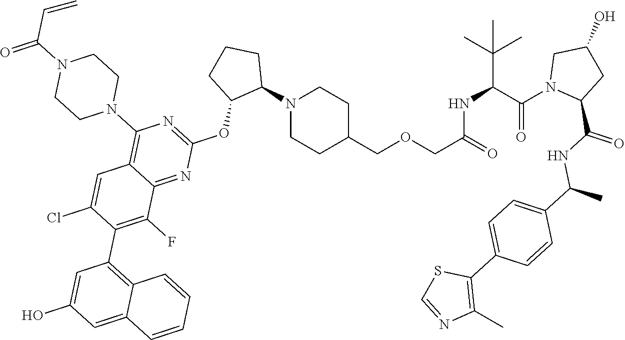 [SMILES: C=CC(=O)N1CCN(c2nc(O[C@@H]3CCC[C@H]3N3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-({1- [(1R,2R)-2-{[6-chloro-8- fluoro-7-(3- hydroxynaphthalen-1-yl)-4-[4- (prop-2-enoyl)piperazin-1- yl]quinazolin-2- yl]oxy}cyclopentyl]piperidin- 4-yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 14 | 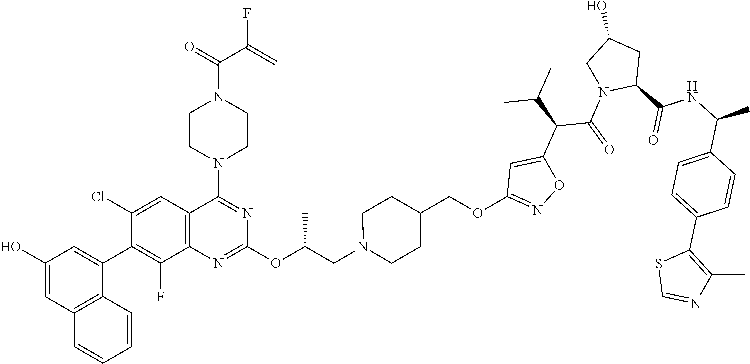 [SMILES: C=C(F)C(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1] | (2S,4R)-1-[(2R)-2-[3-({1- [(2R)-2-({6-chloro-8-fluoro-4- [4-(2-fluoroprop-2- enoyl)piperazin-1-yl]-7-(3- hydroxynaphthalen-1- yl)quinazolin-2- yl}oxy)propyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 15 | 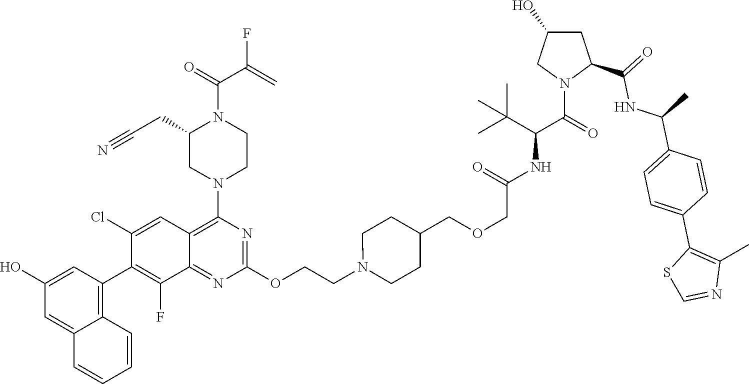 [SMILES: C=C(F)C(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 16 | 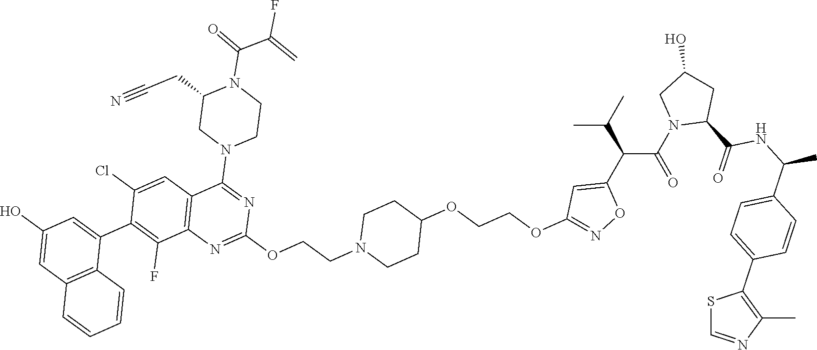 [SMILES: C=C(F)C(=O)N1CCN(c2nc(OCCN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-{3-[2-({1- [2-({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}oxy)ethoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine-2- carboxamide |
| 17 | 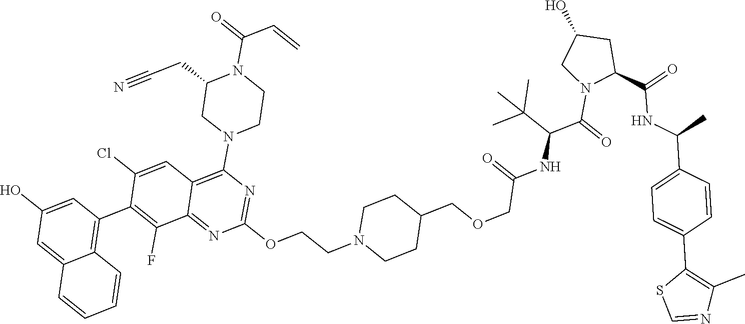 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 18 | 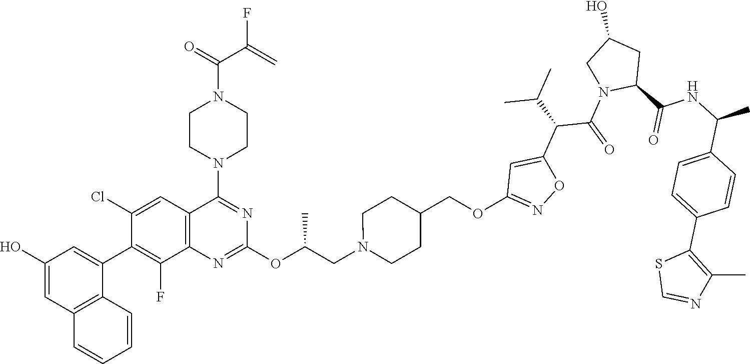 [SMILES: C=C(F)C(=O)N1CCN(c2nc(O[C@H](C)CN3CCC(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1] | (2S,4R)-1-[(2S)-2-[3-({1- [(2R)-2-({6-chloro-8-fluoro-4- [4-(2-fluoroprop-2- enoyl)piperazin-1-yl]-7-(3- hydroxynaphthalen-1- yl)quinazolin-2- yl}oxy)propyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 19 | 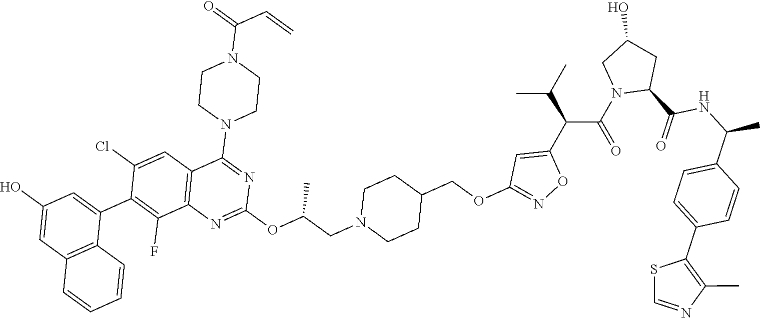 [SMILES: C=CC(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1] | (2S,4R)-1-[(2R)-2-[3-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 20 | 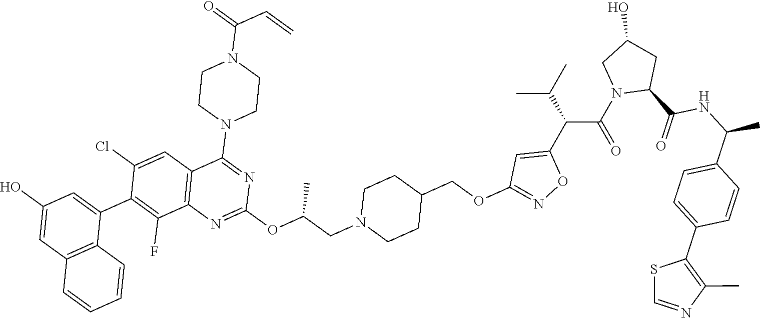 [SMILES: C=CC(=O)N1CCN(c2nc(O[C@H](C)CN3CCC(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1] | (2S,4R)-1-[(2S)-2-[3-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 21 | 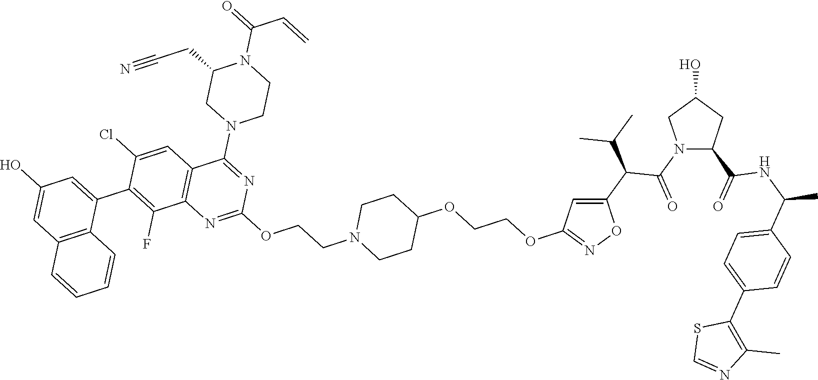 [SMILES: C=CC(=O)N1CCN(c2nc(OCCN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-{3-[2-({1- [2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}oxy)ethoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine-2- carboxamide |
| 22 | 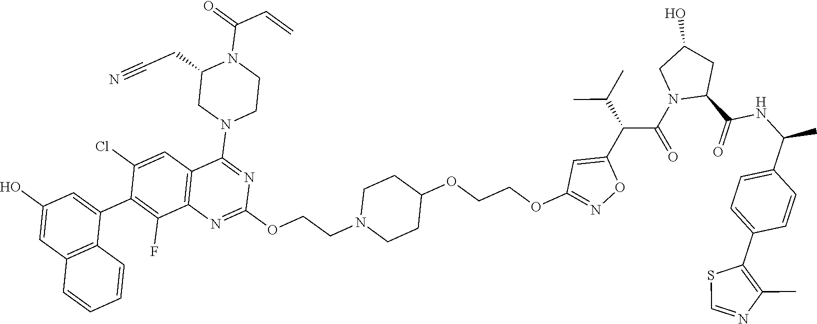 [SMILES: C=CC(=O)N1CCN(c2nc(OCCN3CCC(OCCOc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-{3-[2-({1- [2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}oxy)ethoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine-2- carboxamide |
| 23 | 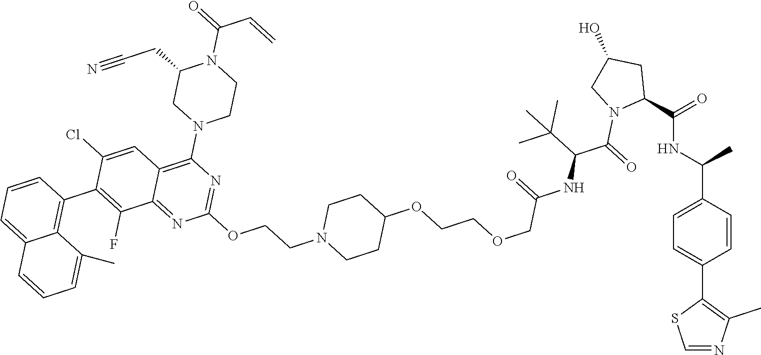 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cccc5cccc(C)c45)c(Cl)cc23)C[C@@H]1CC#N] | (2S,4R)-1-[(2S)-2-{2-[2-({1- [2-({6-chloro-4-[(3S)-3- (cyanomethyl)-4-(prop-2- enoyl)piperazin-1-yl]-8-fluoro- 7-(8-methylnaphthalen-1- yl)quinazolin-2- yl}oxy)ethyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 24 | 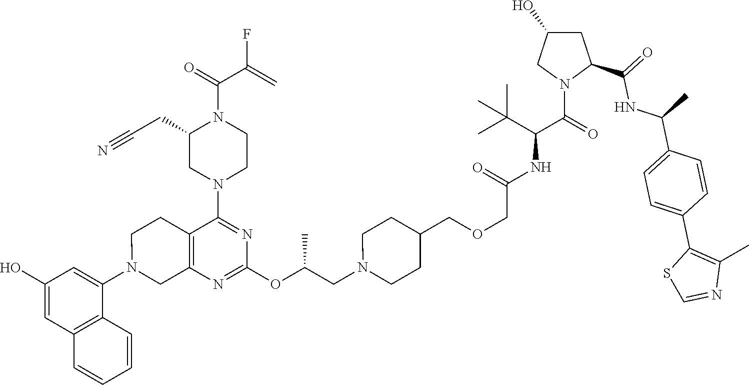 [SMILES: C=C(F)C(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N] | (2S,4R)-1-[(2S)-2-[2-({1- [(2R)-2-({4-[(3S)-3- (cyanomethyl)-4-(2- fluoroprop-2-enoyl)piperazin- 1-yl]-7-(3-hydroxynaphthalen- 1-yl)-5H,6H,7H,8H- pyrido[3,4-d]pyrimidin-2- yl}oxy)propyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 25 | 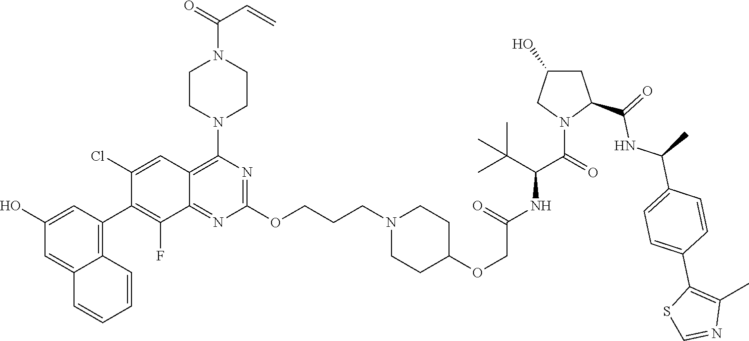 [SMILES: C=CC(=O)N1CCN(c2nc(OCCCN3CCC(OCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-(2-{[1-(3- {[6-chloro-8-fluoro-7-(3- hydroxynaphthalen-1-yl)-4-[4- (prop-2-enoyl)piperazin-1- yl]quinazolin-2- yl]oxy}propyl)piperidin-4- yl]oxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 26 | 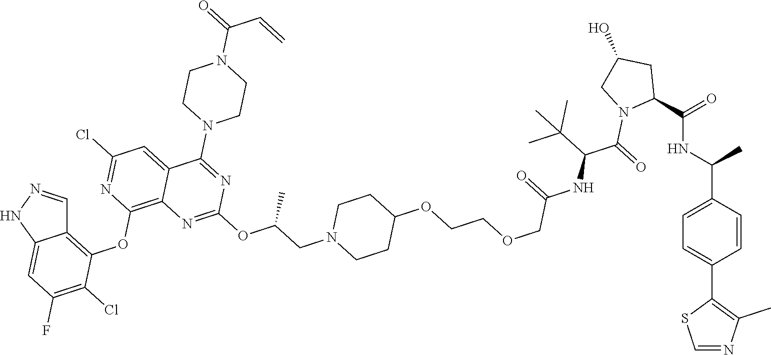 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(Oc4c(Cl)c(F)cc5[nH]ncc45)nc(Cl)cc23)CC1] | (2S,4R)-1-[(2S)-2-{2-[2-({1- [(2R)-2-({6-chloro-8-[(5- chloro-6-fluoro-1H-indazol-4- yl)oxy]-4-[4-(prop-2- enoyl)piperazin-1- yl]pyrido[3,4-d]pyrimidin-2- yl}oxy)propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 27 | 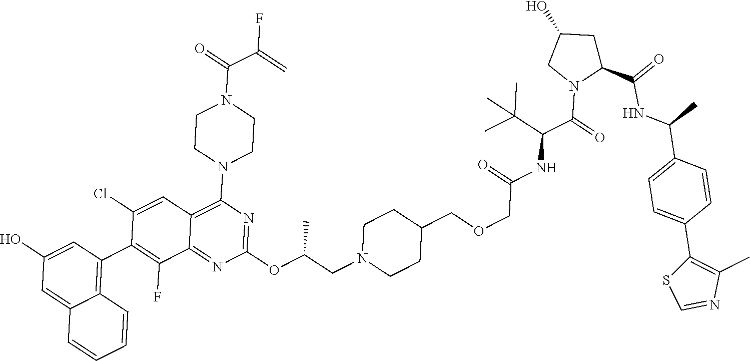 [SMILES: C=C(F)C(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-({1- [(2R)-2-({6-chloro-8-fluoro-4- [4-(2-fluoroprop-2- enoyl)piperazin-1-yl]-7-(3- hydroxynaphthalen-1- yl)quinazolin-2- yl}oxy)propyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 28 | 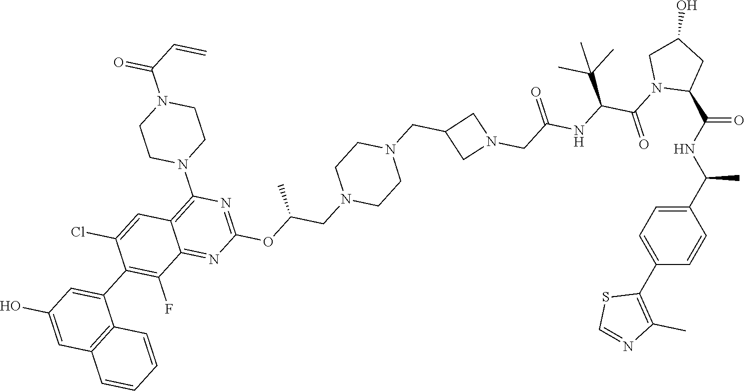 [SMILES: C=CC(=O)N1CCN(c2nc(O[C@H](C)CN3CCN(CC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1] | (2S,4R)-1-[(2S)-2-{2-[3-({4- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperazin-1- yl}methyl)azetidin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 29 | 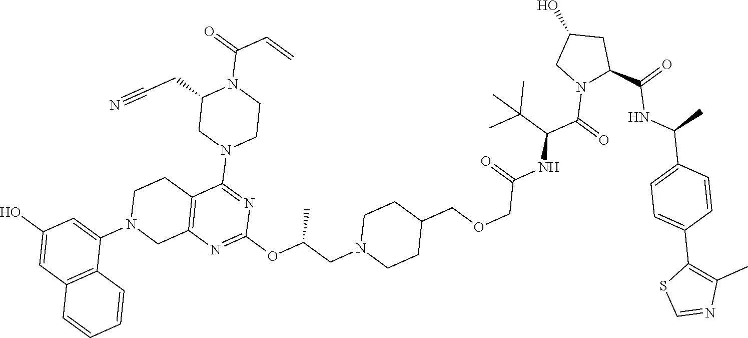 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N] | (2S,4R)-1-[(2S)-2-[2-({1- [(2R)-2-({4-[(3S)-3- (cyanomethyl)-4-(prop-2- enoyl)piperazin-1-yl]-7-(3- hydroxynaphthalen-1-yl)- 5H,6H,7H,8H-pyrido[3,4- d]pyrimidin-2- yl}oxy)propyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 30 | 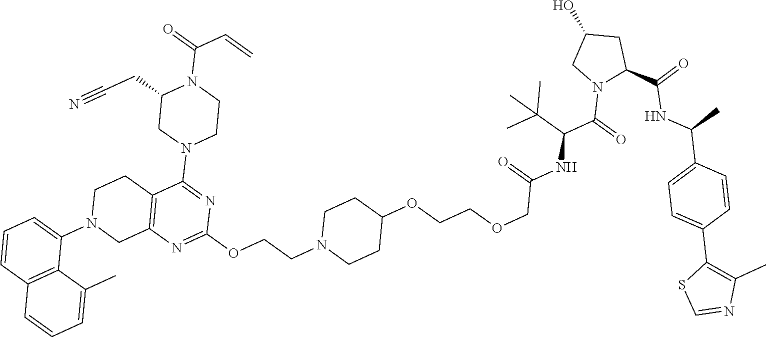 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cccc4cccc(C)c24)C3)C[C@@H]1CC#N] | (2S,4R)-1-[(2S)-2-{2-[2-({1- [2-({4-[(3S)-3-(cyanomethyl)- 4-(prop-2-enoyl)piperazin-1- yl]-7-(8-methylnaphthalen-1- yl)-5H,6H,7H,8H-pyrido[3,4- d]pyrimidin-2- yl}oxy)ethyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 31 | 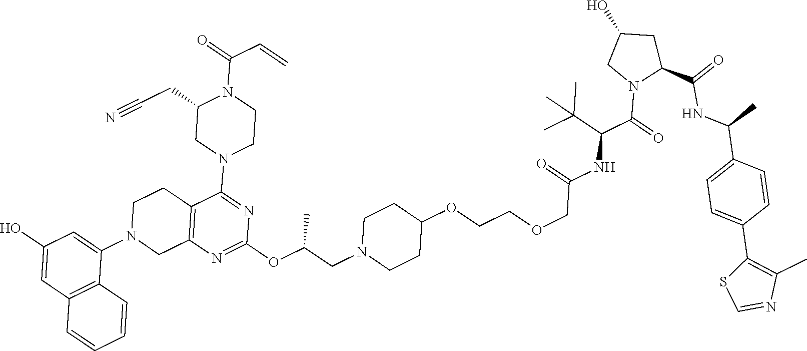 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N] | (2S,4R)-1-[(2S)-2-{2-[2-({1- [(2R)-2-({4-[(3S)-3- (cyanomethyl)-4-(prop-2- enoyl)piperazin-1-yl]-7-(3- hydroxynaphthalen-1-yl)- 5H,6H,7H,8H-pyrido[3,4- d]pyrimidin-2- yl}oxy)propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 32 | 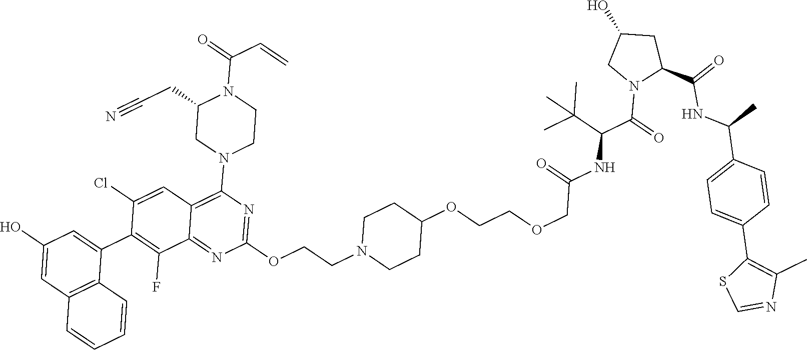 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-{2-[2-({1- [2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 33 | 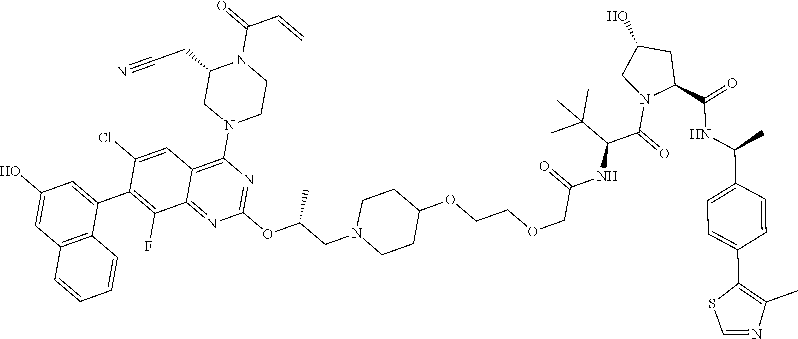 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-{2-[2-({1- [(2R)-2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 34 | 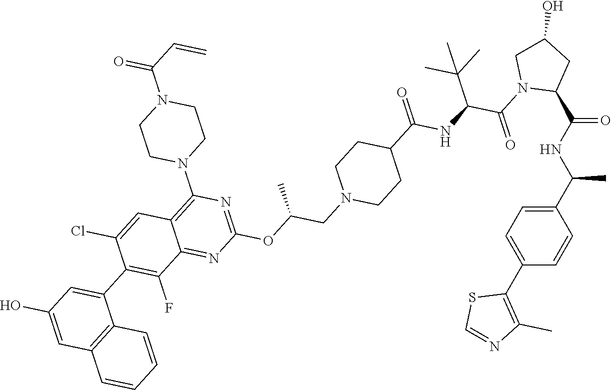 [SMILES: C=CC(=O)N1CCN(c2nc(O[C@H](C)CN3CCC(C(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | 1-[(2R)-2-{[6-chloro-8-fluoro- 7-(3-hydroxynaphthalen-1-yl)- 4-[4-(prop-2-enoyl)piperazin- 1-yl]quinazolin-2- yl]oxy}propyl]-N-[(2S)-1- [(2S,4R)-4-hydroxy-2-{[(1S)- 1-[4-(4-methyl-1,3-thiazol-5- yl)phenyl]ethyl]carbamoyl} pyrrolidin-1-yl]-3,3-dimethyl-1- oxobutan-2-yl]piperidine-4- carboxamide |
| 35 | 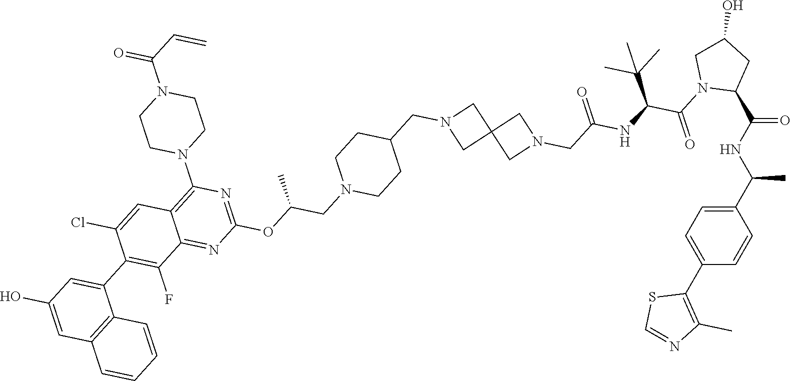 [SMILES: C=CC(=O)N1CCN(c2nc(O[C@H](C)CN3CCC(CN4CC5(CN(CC(=O)N[C@H](C(=O)N6C[C@H](O)C[C@H]6C(=O)N[C@@H](C)c6ccc(-c7scnc7C)cc6)C(C)(C)C)C5)C4)CC3)nc3c(F)c(-c4cc(O)cc5ccccc45)c(Cl)cc23)CC1] | (2S,4R)-1-[(2S)-2-{2-[6-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}methyl)-2,6- diazaspiro[3.3]heptan-2- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 36 | 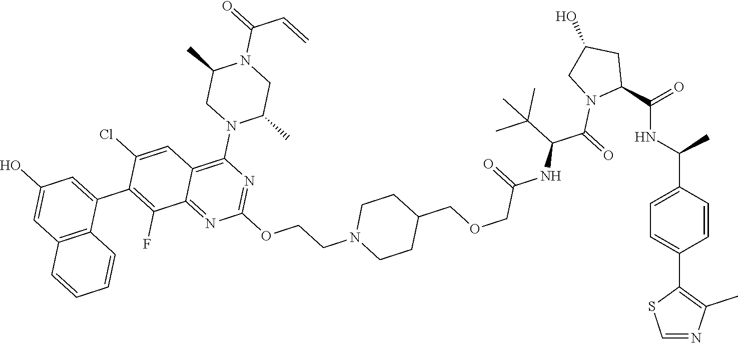 [SMILES: C=CC(=O)N1C[C@H](C)N(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@H]1C] | (2S,4R)-1-[(2S)-2-[2-({1-[2- ({6-chloro-4-[(2S,5R)-2,5- dimethyl-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 37 | 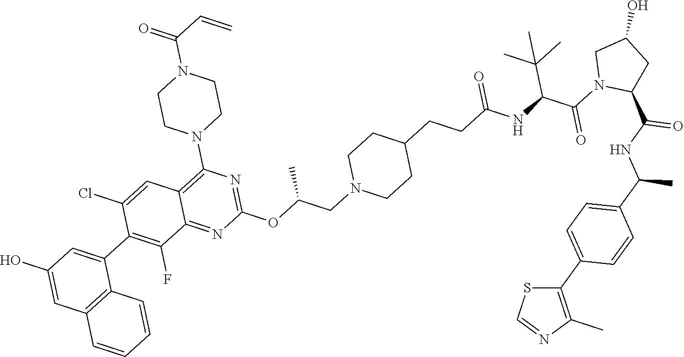 [SMILES: C=CC(=O)N1CCN(c2nc(O[C@H](C)CN3CCC(CCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-(3-{1-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}propanamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 38 | 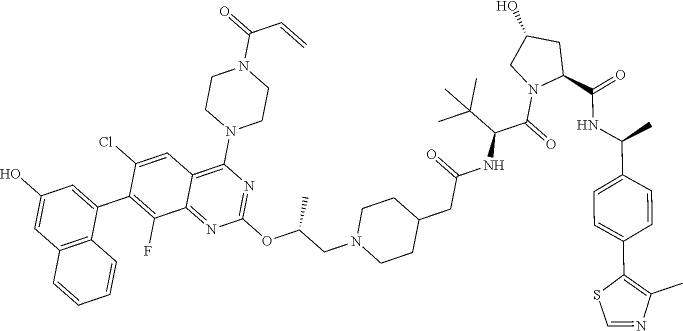 [SMILES: C=CC(=O)N1CCN(c2nc(O[C@H](C)CN3CCC(CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-(2-{1-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 39 | 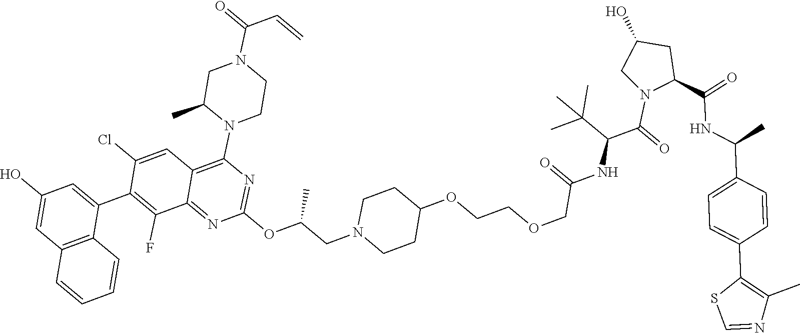 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)[C@@H](C)C1] | (2S,4R)-1-[(2S)-2-{2-[2-({1- [(2R)-2-{[6-chloro-8-fluoro-7- (3-hydroxynaphthalen-1-yl)-4- [(2S)-2-methyl-4-(prop-2- enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 40 | 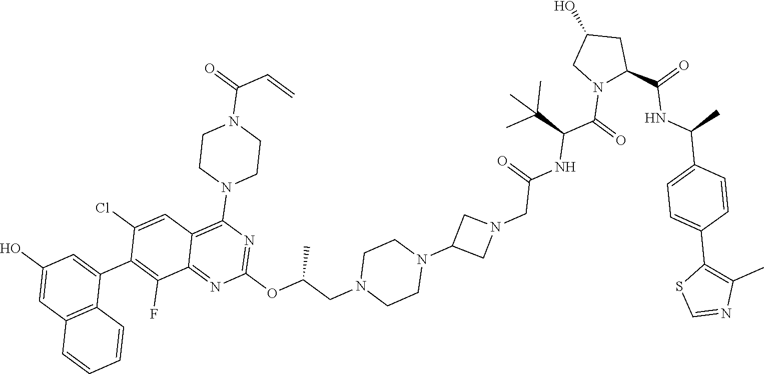 [SMILES: C=CC(=O)N1CCN(c2nc(O[C@H](C)CN3CCN(C4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1] | (2S,4R)-1-[(2S)-2-[2-(3-{4- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperazin-1- yl}azetidin-1-yl)acetamido]- 3,3-dimethylbutanoyl]-4- hydroxy-N-[(1S)-1-[4-(4- methyl-1,3-thiazol-5- yl)phenyl]ethyl]pyrrolidine-2- carboxamide |
| 41 | 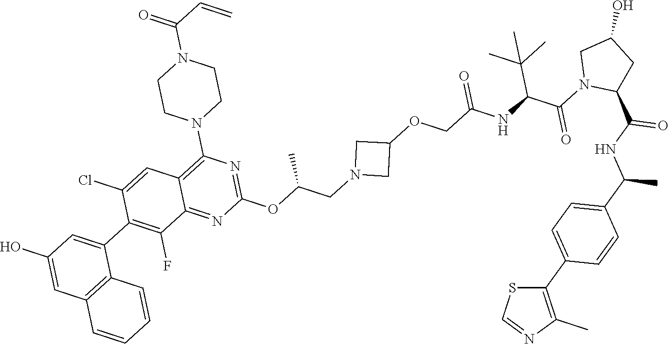 [SMILES: C=CC(=O)N1CCN(c2nc(O[C@H](C)CN3CC(OCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)CC1] | (2S,4R)-1-[(2S)-2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]azetidin-3- yl}oxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 42 | 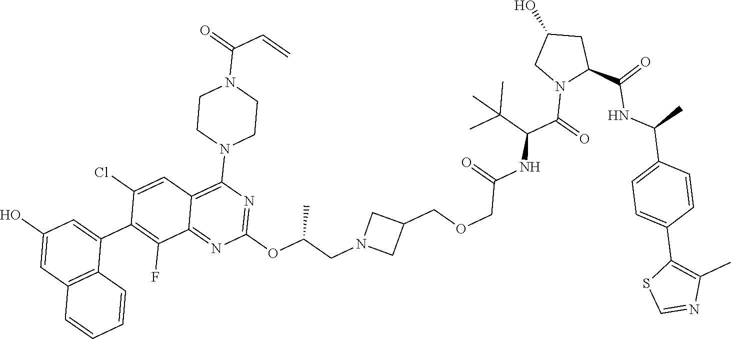 [SMILES: C=CC(=O)N1CCN(c2nc(O[C@H](C)CN3CC(COCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)CC1] | (2S,4R)-1-[(2S)-2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]azetidin-3- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 43 | 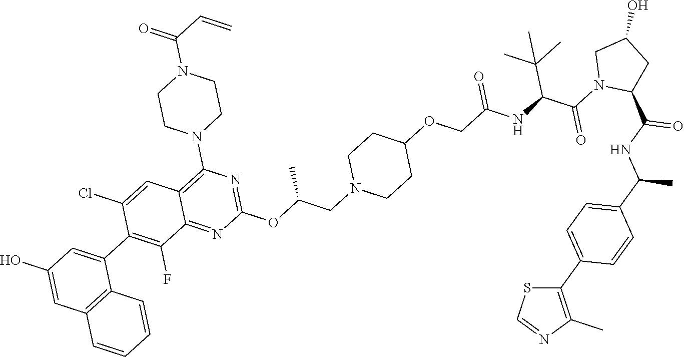 [SMILES: C=CC(=O)N1CCN(c2nc(O[C@H](C)CN3CCC(OCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 44 | 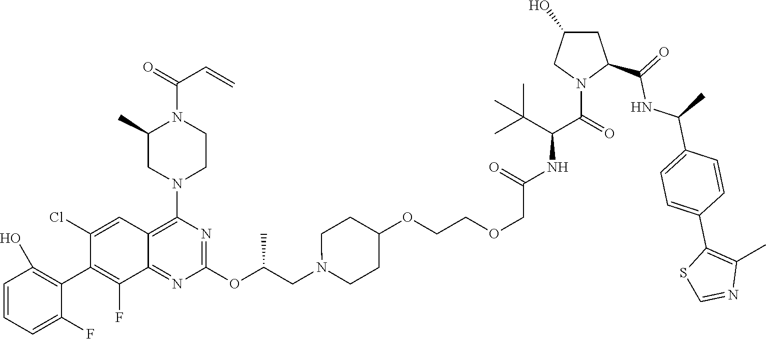 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(O)cccc4F)c(Cl)cc23)C[C@H]1C] | (2S,4R)-1-[(2S)-2-{2-[2-({1- [(2R)-2-{[6-chloro-8-fluoro-7- (2-fluoro-6-hydroxyphenyl)-4- [(3R)-3-methyl-4-(prop-2- enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 45 | 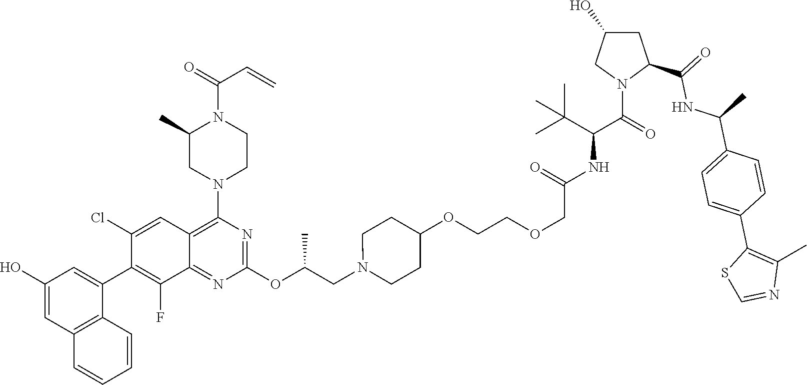 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@H]1C] | (2S,4R)-1-[(2S)-2-{2-[2-({1- [(2R)-2-{[6-chloro-8-fluoro-7- (3-hydroxynaphthalen-1-yl)-4- [(3R)-3-methyl-4-(prop-2- enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 46 | 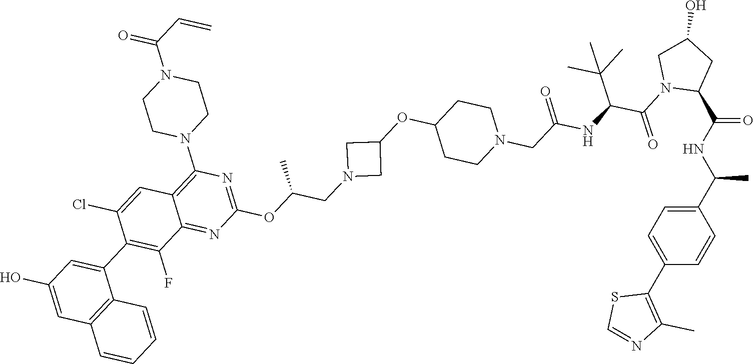 [SMILES: C=CC(=O)N1CCN(c2nc(O[C@H](C)CN3CC(OC4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1] | (2S,4R)-1-[(2S)-2-{2-[4-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]azetidin-3- yl}oxy)piperidin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 47 | 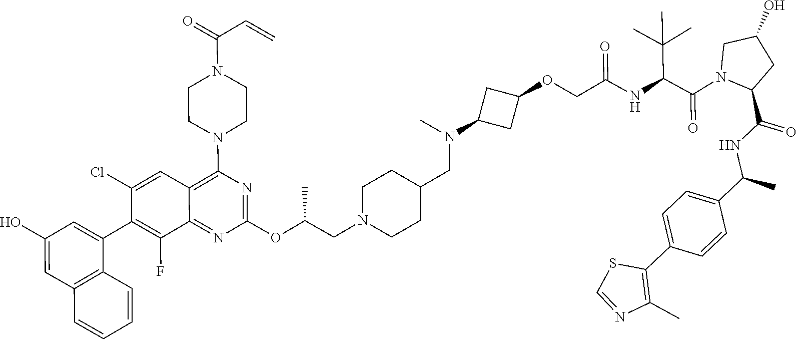 [SMILES: C=CC(=O)N1CCN(c2nc(O[C@H](C)CN3CCC(CN(C)[C@H]4C[C@@H](OCC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1] | (2S,4R)-1-[(2S)-3,3-dimethyl- 2-(2-[(1s,3s)-3-[({1-[(2R)-2- {[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}methyl)(methyl)amino] cyclobutoxy]acetamido}butanoyl]- 4-hydroxy-N-[(1S)-1-[4-(4- methyl-1,3-thiazol-5- yl)phenyl]ethyl]pyrrolidine-2- carboxamide |
| 48 | 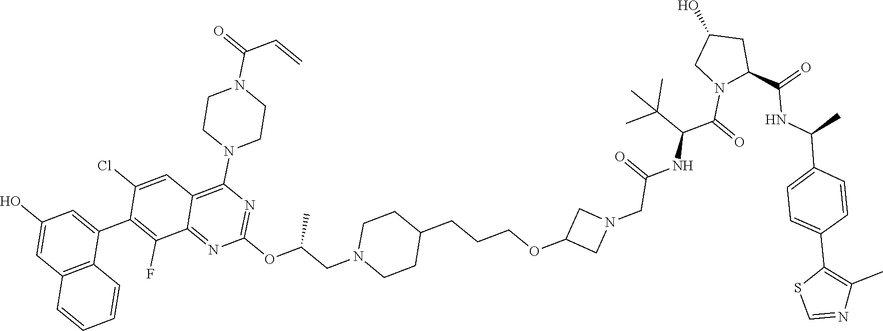 [SMILES: C=CC(=O)N1CCN(c2nc(O[C@H](C)CN3CCC(CCCOC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1] | (2S,4R)-1-[(2S)-2-{2-[3-(3-{1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}propoxy)azetidin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 49 | 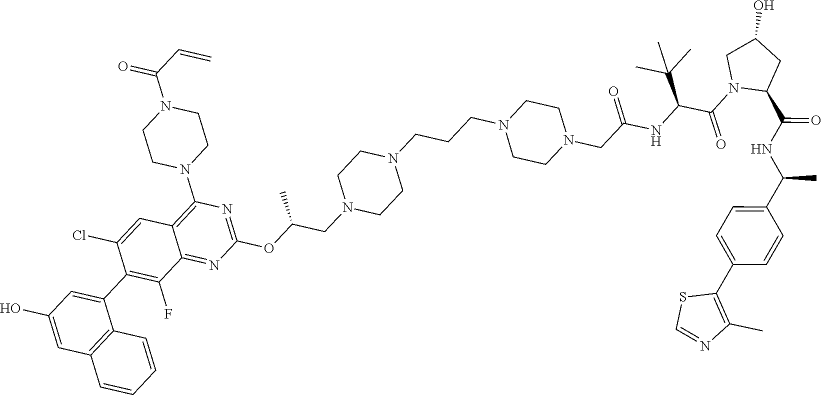 [SMILES: C=CC(=O)N1CCN(c2nc(O[C@H](C)CN3CCN(CCCN4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1] | (2S,4R)-1-[(2S)-2-{2-[4-(3-{4- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperazin-1- yl}propyl)piperazin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 50 | 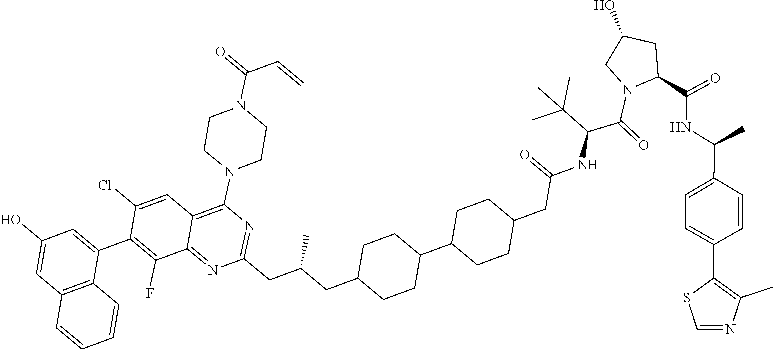 [SMILES: C=CC(=O)N1CCN(c2nc(C[C@H](C)CC3CCC(C4CCC(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1] | (2S,4R)-1-[(2S)-2-(2-{1′-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]-[4,4′- bipiperidin]-1-yl}acetamido)- 3,3-dimethylbutanoyl]-4- hydroxy-N-[(1S)-1-[4-(4- methyl-1,3-thiazol-5- yl)phenyl]ethyl]pyrrolidine-2- carboxamide |
| 51 | 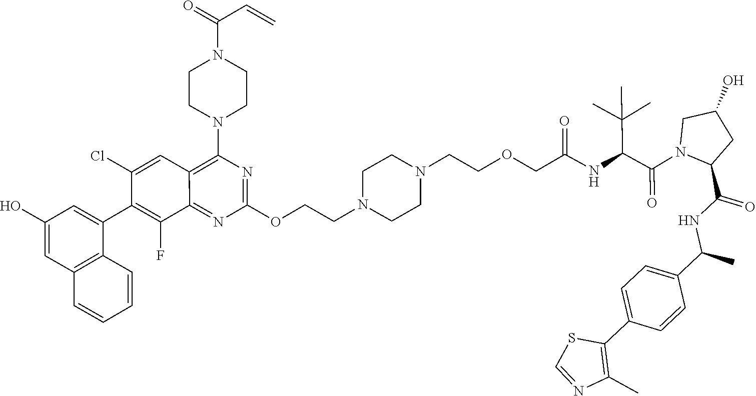 [SMILES: C=CC(=O)N1CCN(c2nc(OCCN3CCN(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-(2-{2-[4-(2- {[6-chloro-8-fluoro-7-(3- hydroxynaphthalen-1-yl)-4-[4- (prop-2-enoyl)piperazin-1- yl]quinazolin-2- yl]oxy}ethyl)piperazin-1- yl]ethoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 52 | 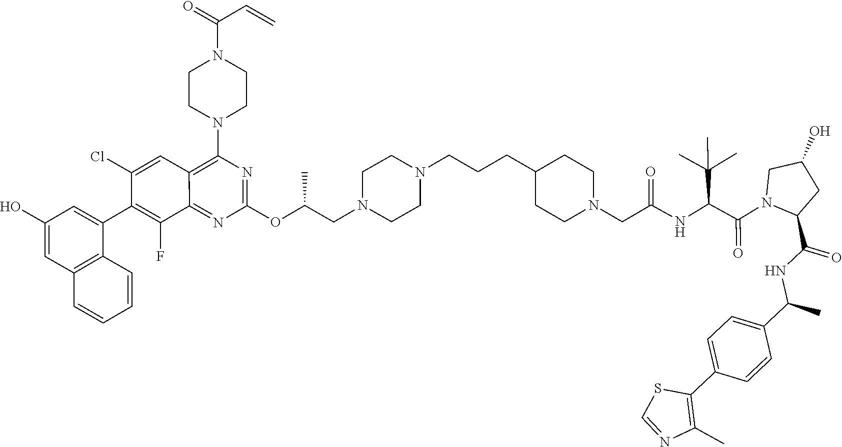 [SMILES: C=CC(=O)N1CCN(c2nc(O[C@H](C)CN3CCN(CCCC4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1] | (2S,4R)-1-[(2S)-2-{2-[4-(3-{4- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperazin-1- yl}propyl)piperidin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 53 | 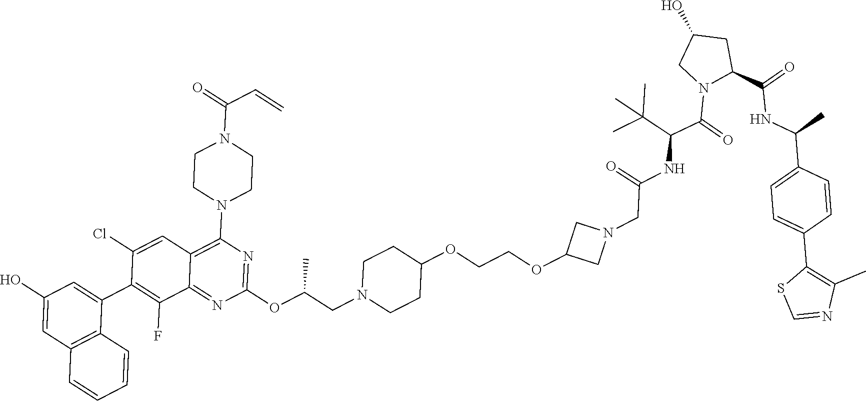 [SMILES: C=CC(=O)N1CCN(c2nc(O[C@H](C)CN3CCC(OCCOC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1] | (2S,4R)-1-[(2S)-2-(2-{3-[2- ({1-[(2R)-2-{[6-chloro-8- fluoro-7-(3- hydroxynaphthalen-1-yl)-4-[4- (prop-2-enoyl)piperazin-1- yl]quinazolin-2- yl]oxy}propyl]piperidin-4- yl}oxy)ethoxy]azetidin-1- yl}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 54 | 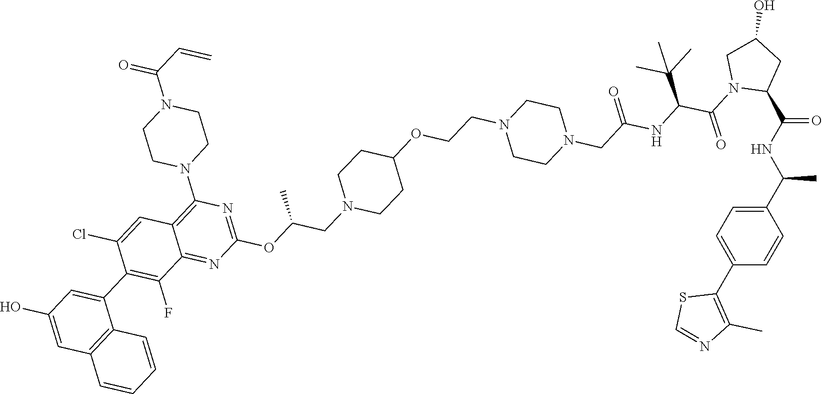 [SMILES: C=CC(=O)N1CCN(c2nc(O[C@H](C)CN3CCC(OCCN4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1] | (2S,4R)-1-[(2S)-2-(2-{4-[2- ({1-[(2R)-2-{[6-chloro-8- fluoro-7-(3- hydroxynaphthalen-1-yl)-4-[4- (prop-2-enoyl)piperazin-1- yl]quinazolin-2- yl]oxy}propyl]piperidin-4- yl}oxy)ethyl]piperazin-1- yl}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 55 | 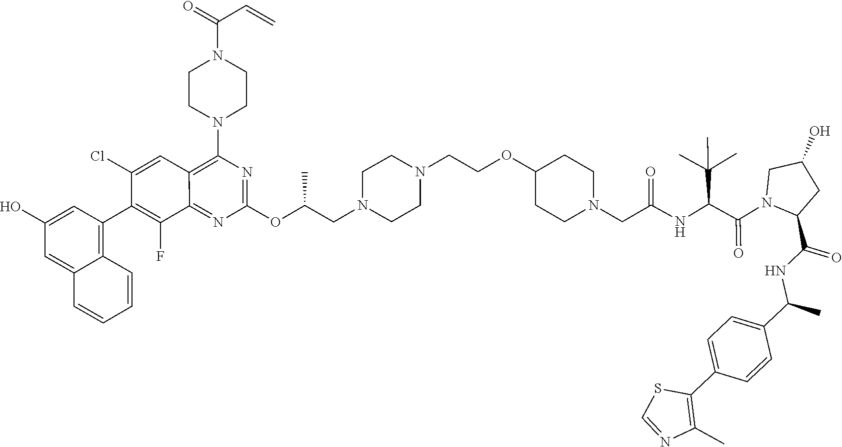 [SMILES: C=CC(=O)N1CCN(c2nc(O[C@H](C)CN3CCN(CCOC4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1] | (2S,4R)-1-[(2S)-2-{2-[4-(2-{4- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperazin-1- yl}ethoxy)piperidin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 56 | 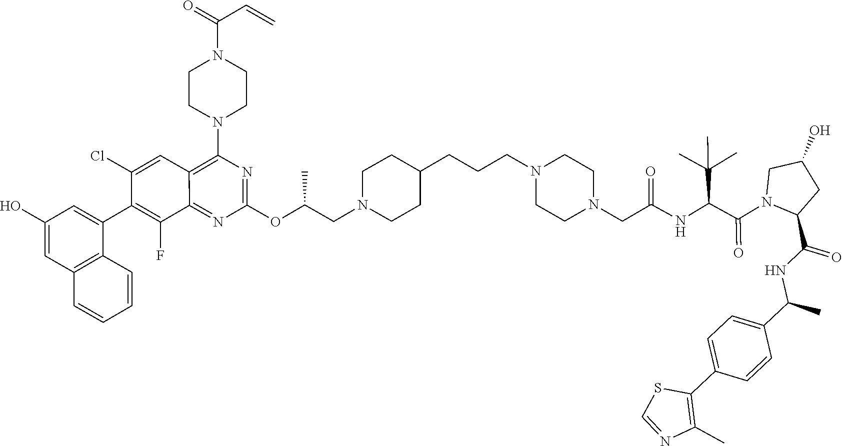 [SMILES: C=CC(=O)N1CCN(c2nc(O[C@H](C)CN3CCC(CCCN4CCN(CC(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)CC1] | (2S,4R)-1-[(2S)-2-{2-[4-(3-{1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}propyl)piperazin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 57 | 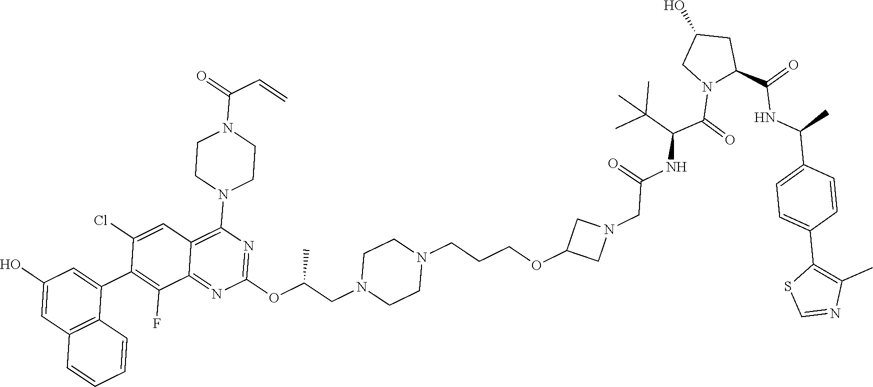 [SMILES: C=CC(=O)N1CCN(c2nc(O[C@H](C)CN3CCN(CCCOC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1] | (2S,4R)-1-[(2S)-2-{2-[3-(3-{4- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperazin-1- yl}propoxy)azetidin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 58 | 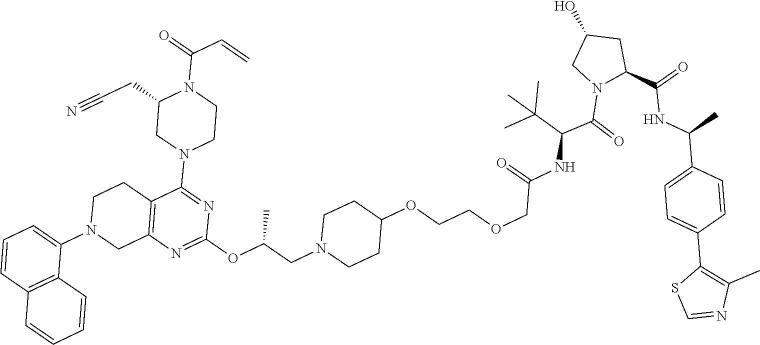 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cccc4ccccc24)C3)C[C@@H]1CC#N] | (2S,4R)-1-[(2S)-2-{2-[2-({1- [(2R)-2-({4-[(3S)-3- (cyanomethyl)-4-(prop-2- enoyl)piperazin-1-yl]-7- (naphthalen-1-yl)- 5H,6H,7H,8H-pyrido[3,4- d]pyrimidin-2- yl}oxy)propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 59 | 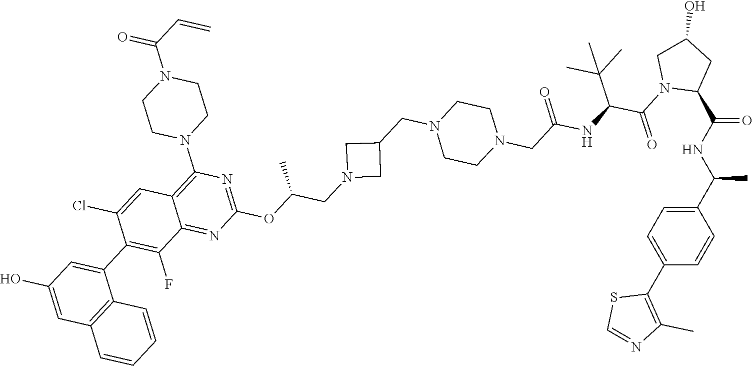 [SMILES: C=CC(=O)N1CCN(c2nc(O[C@H](C)CN3CC(CN4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1] | (2S,4R)-1-[(2S)-2-{2-[4-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]azetidin-3- yl}methyl)piperazin-1- yl]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 60 | 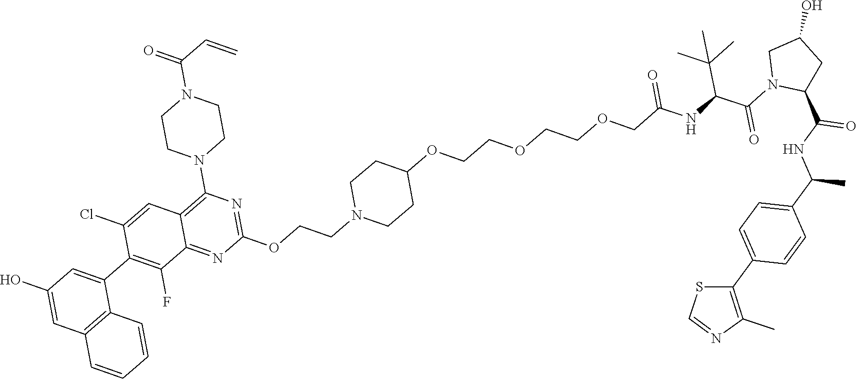 [SMILES: C=CC(=O)N1CCN(c2nc(OCCN3CCC(OCCOCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-{2-[2-(2- {[1-(2-{[6-chloro-8-fluoro-7- (3-hydroxynaphthalen-1-yl)-4- [4-(prop-2-enoyl)piperazin-1- yl]quinazolin-2- yl]oxy}ethyl)piperidin-4- yl]oxy}ethoxy)ethoxy]acetamido}- 3,3-dimethylbutanoyl]-4- hydroxy-N-[(1S)-1-[4-(4- methyl-1,3-thiazol-5- yl)phenyl]ethyl]pyrrolidine-2- carboxamide |
| 61 | 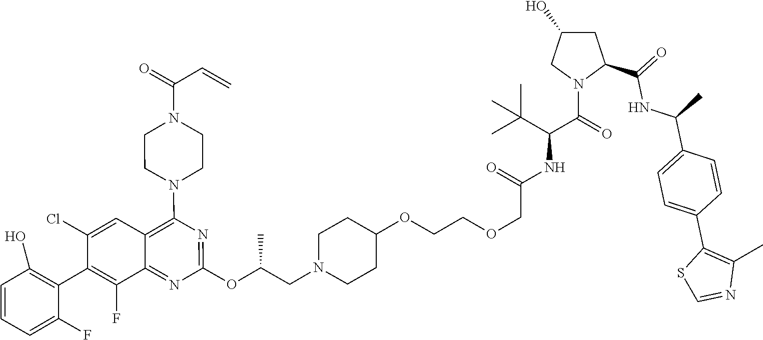 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(O)cccc4F)c(Cl)cc23)CC1] | (2S,4R)-1-[(2S)-2-{2-[2-({1- [(2R)-2-{[6-chloro-8-fluoro-7- (2-fluoro-6-hydroxyphenyl)-4- [4-(prop-2-enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 62 | 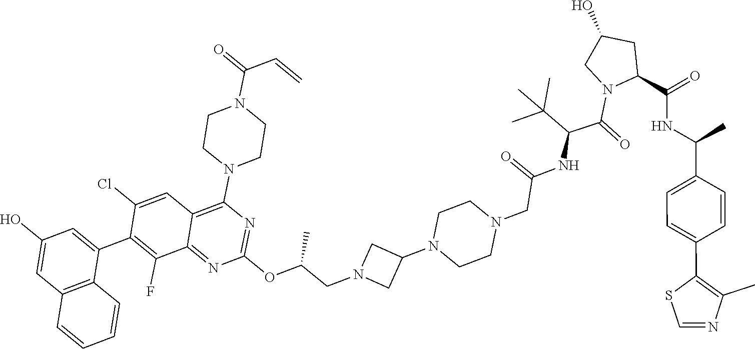 [SMILES: C=CC(=O)N1CCN(c2nc(O[C@H](C)CN3CC(N4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1] | (2S,4R)-1-[(2S)-2-[2-(4-{1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]azetidin-3- yl}piperazin-1-yl)acetamido]- 3,3-dimethylbutanoyl]-4- hydroxy-N-[(1S)-1-[4-(4- methyl-1,3-thiazol-5- yl)phenyl]ethyl]pyrrolidine-2- carboxamide |
| 63 | 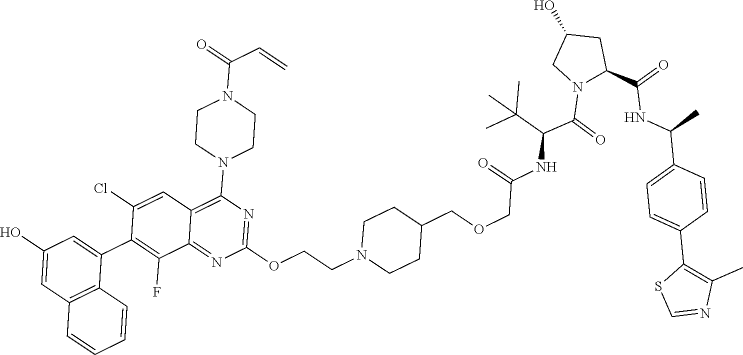 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-(2-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 64 | 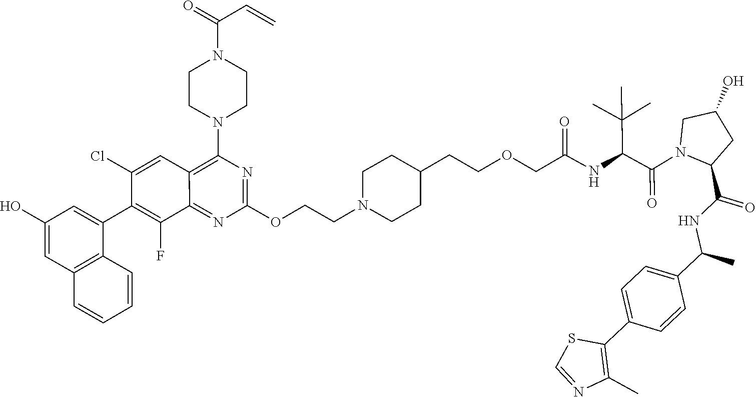 [SMILES: C=CC(=O)N1CCN(c2nc(OCCN3CCC(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-(2-{2-[l-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]ethoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 65 | 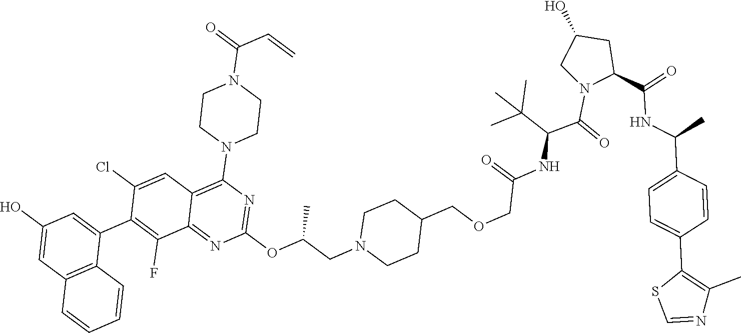 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 66 | 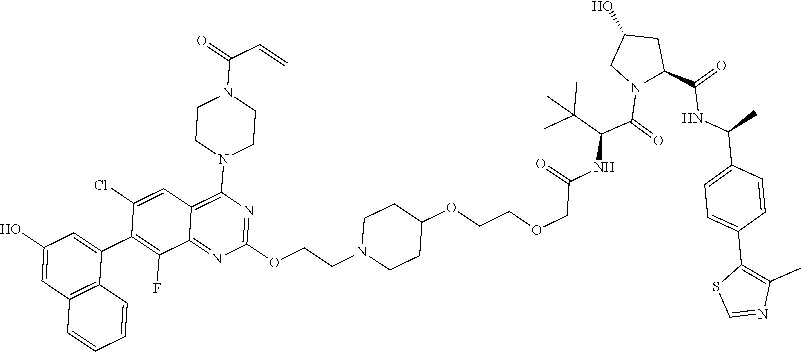 [SMILES: C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-(2-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]oxy}ethoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 67 | 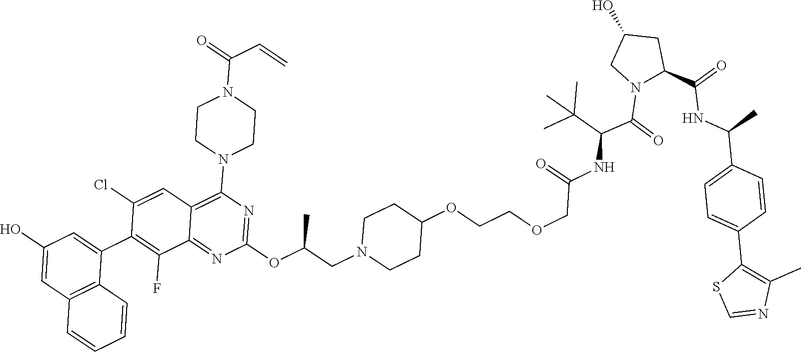 [SMILES: C=CC(=O)N1CCN(c2nc(O[C@@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-{2-[2-({1- [(2S)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 68 | 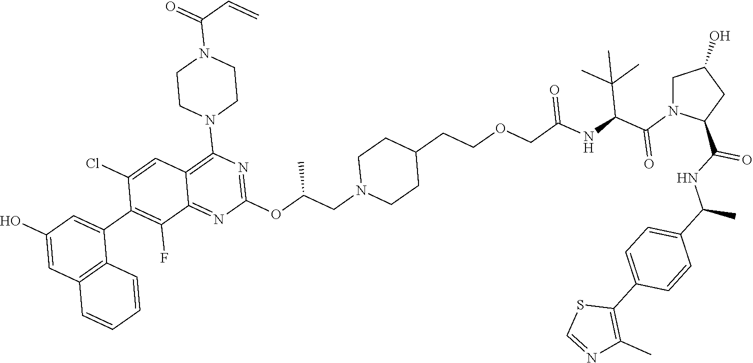 [SMILES: C=CC(=O)N1CCN(c2nc(O[C@H](C)CN3CCC(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[2-(2-{1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}ethoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 69 | 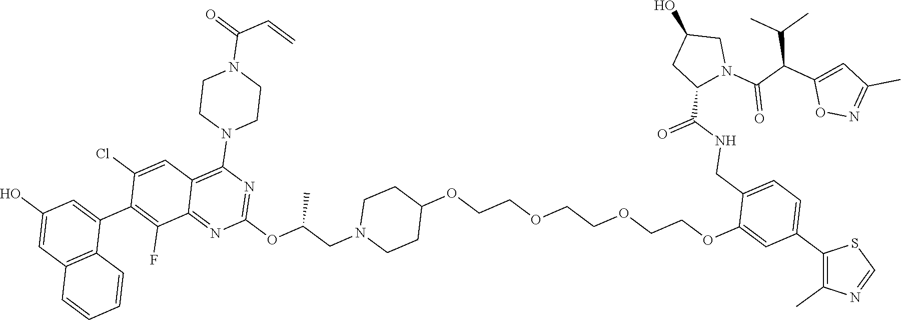 [SMILES: C=CC(=O)N1CCN(c2nc(O[C@H](C)CN3CCC(OCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1] | (2S,4R)-N-{[2-(2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]ethoxy}ethoxy)- 4-(4-methyl-1,3-thiazol-5- yl)phenyl]methyl}-4-hydroxy- 1-[(2S)-3-methyl-2-(3-methyl- 1,2-oxazol-5- yl)butanoyl]pyrrolidine-2- carboxamide |
| 70 | 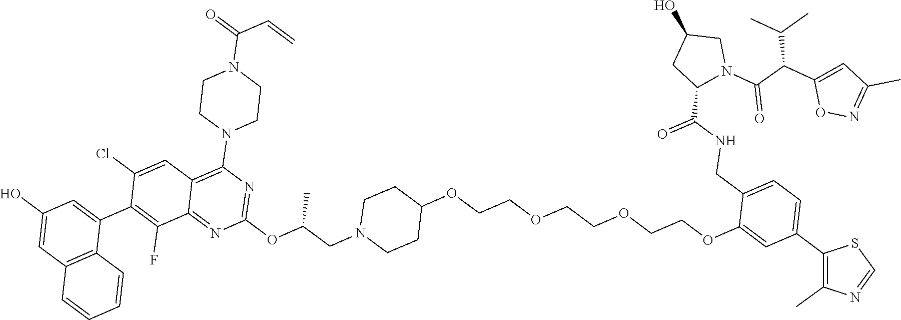 [SMILES: C=CC(=O)N1CCN(c2nc(O[C@H](C)CN3CCC(OCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1] | (2S,4R)-N-{[2-(2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]ethoxy}ethoxy)- 4-(4-methyl-1,3-thiazol-5- yl)phenyl]methyl}-4-hydroxy- 1-[(2R)-3-methyl-2-(3-methyl- 1,2-oxazol-5- yl)butanoyl]pyrrolidine-2- carboxamide |
| 71 | 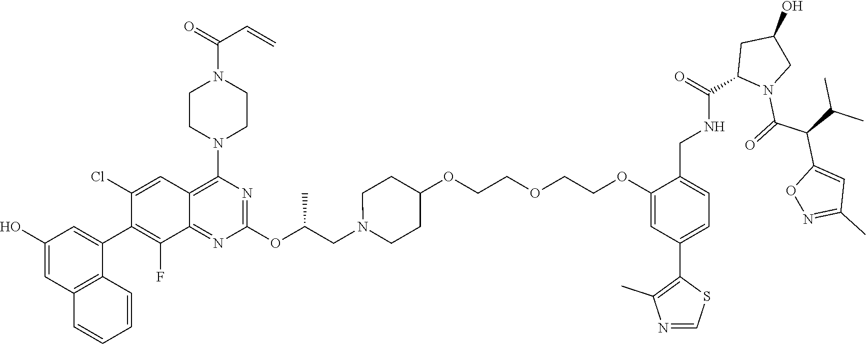 [SMILES: C=CC(=O)N1CCN(c2nc(O[C@H](C)CN3CCC(OCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1] | (2S,4R)-N-[(2-{2-[2-({1-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}oxy)ethoxy]ethoxy}-4-(4- methyl-1,3-thiazol-5- yl)phenyl)methyl]-4-hydroxy- 1-[(2S)-3-methyl-2-(3-methyl- 1,2-oxazol-5- yl)butanoyl]pyrrolidine-2- carboxamide |
| 72 | 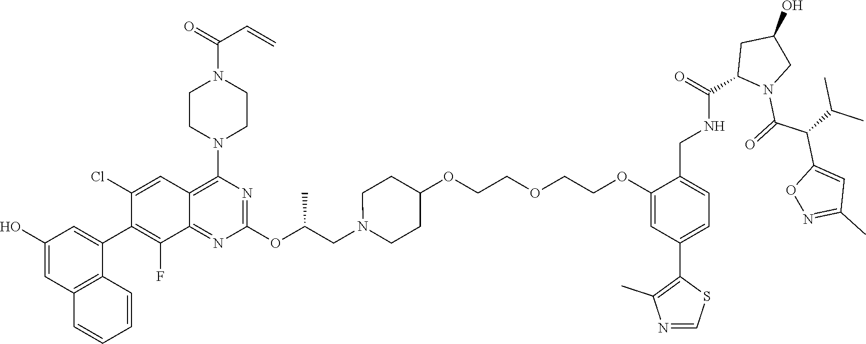 [SMILES: C=CC(=O)N1CCN(c2nc(O[C@H](C)CN3CCC(OCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1] | (2S,4R)-N-[(2-{2-[2-({1-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}oxy)ethoxy]ethoxy}-4-(4- methyl-1,3-thiazol-5- yl)phenyl)methyl]-4-hydroxy- 1-[(2R)-3-methyl-2-(3-methyl- 1,2-oxazol-5- yl)butanoyl]pyrrolidine-2- carboxamide |
| 73 | 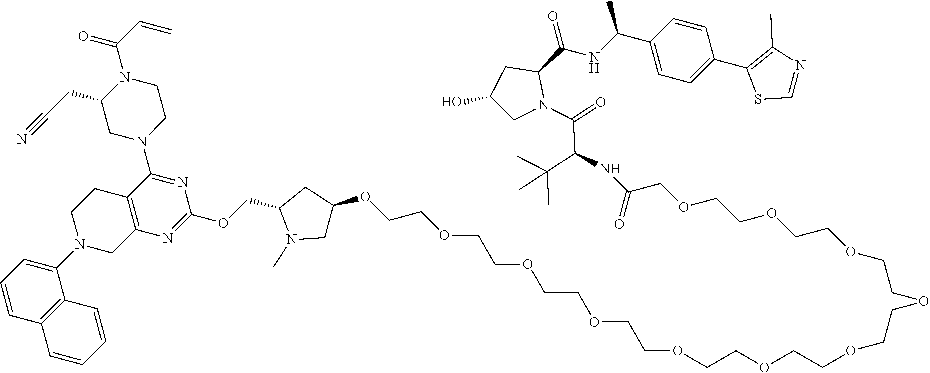 [SMILES: C=CC(=O)N1CCN(c2nc(OC[C@@H]3C[C@@H](OCCOCCOCCOCCOCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CN3C)nc3c2CCN(c2cccc4ccccc24)C3)C[C@@H]1CC#N] | (2S,4R)-1-[(2S)-2-(32- {[(3R,5S)-5-[({4-[(3S)-3- (cyanomethyl)-4-(prop-2- enoyl)piperazin-1-yl]-7- (naphthalen-1-yl)- 5H,6H,7H,8H-pyrido[3,4- d]pyrimidin-2-yl}oxy)methyl]- 1-methylpyrrolidin-3-yl]oxy}- 3,6,9,12,15,18,21,24,27,30- decaoxadotriacontanamido)- 3,3-dimethylbutanoyl]-4- hydroxy-N-[(1S)-1-[4-(4- methyl-1,3-thiazol-5- yl)phenyl]ethyl]pyrrolidine-2- carboxamide |
| 74 | 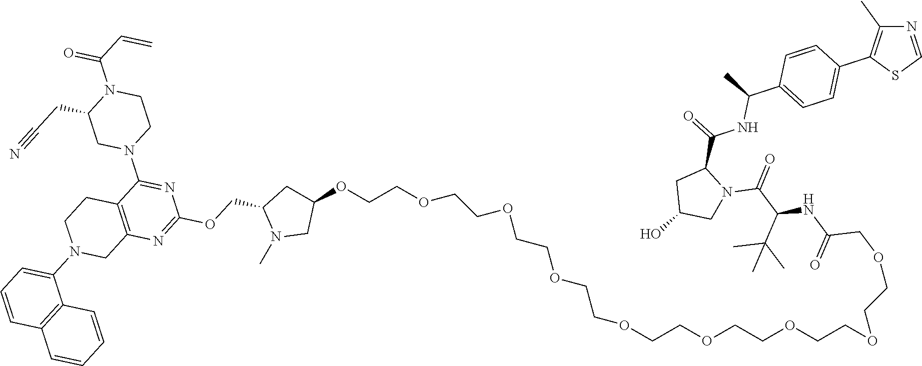 [SMILES: C=CC(=O)N1CCN(c2nc(OC[C@@H]3C[C@@H](OCCOCCOCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CN3C)nc3c2CCN(c2cccc4ccccc24)C3)C[C@@H]1CC#N] | (2S,4R)-1-[(2S)-2-(26- {[(3R,5S)-5-[({4-[(3S)-3- (cyanomethyl)-4-(prop-2- enoyl)piperazin-1-yl]-7- (naphthalen-1-yl)- 5H,6H,7H,8H-pyrido[3,4- d]pyrimidin-2-yl}oxy)methyl]- 1-methylpyrrolidin-3-yl]oxy}- 3,6,9,12,15,18,21,24- octaoxahexacosanamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 75 | 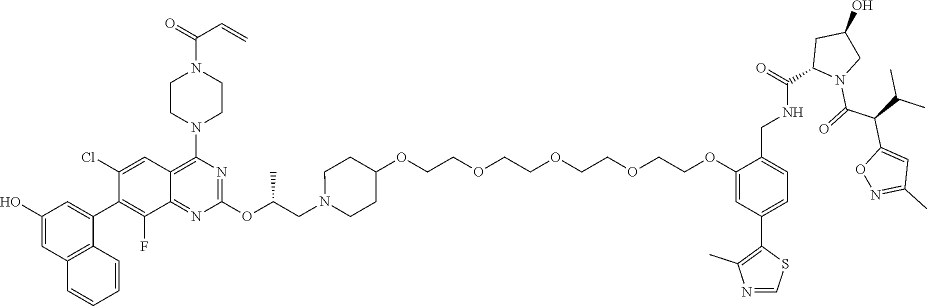 [SMILES: C=CC(=O)N1CCN(c2nc(O[C@H](C)CN3CCC(OCCOCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1] | (2S,4R)-N-({2-[2-(2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]ethoxy}ethoxy) ethoxy]-4-(4-methyl-1,3- thiazol-5-yl)phenyl}methyl)-4- hydroxy-1-[(2S)-3-methyl-2- (3-methyl-1,2-oxazol-5- yl)butanoyl]pyrrolidine-2- carboxamide |
| 76 | 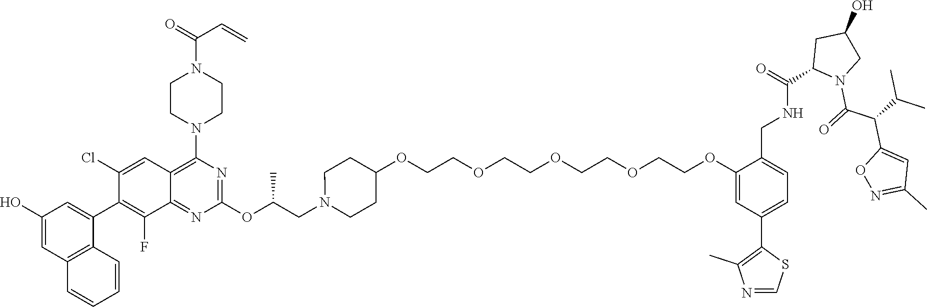 [SMILES: C=CC(=O)N1CCN(c2nc(O[C@H](C)CN3CCC(OCCOCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1] | (2S,4R)-N-({2-[2-(2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]ethoxy}ethoxy) ethoxy]-4-(4-methyl-1,3- thiazol-5-yl)phenyl}methyl)-4- hydroxy-1-[(2R)-3-methyl-2- (3-methyl-1,2-oxazol-5- yl)butanoyl]pyrrolidine-2- carboxamide |
| 77 | 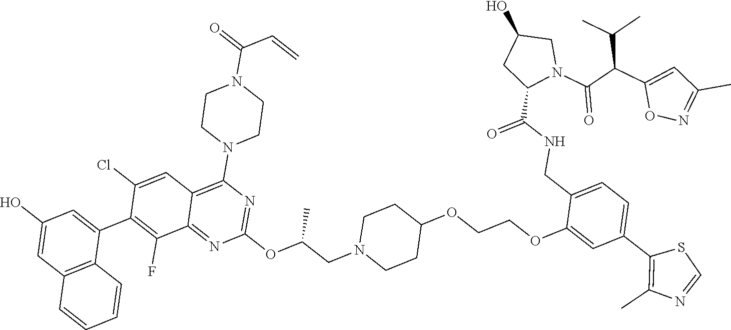 [SMILES: C=CC(=O)N1CCN(c2nc(O[C@H](C)CN3CCC(OCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1] | (2S,4R)-N-({2-[2-({1-[(2R)-2- {[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}oxy)ethoxy]-4-(4-methyl- 1,3-thiazol-5- yl)phenyl}methyl)-4-hydroxy- 1-[(2S)-3-methyl-2-(3-methyl- 1,2-oxazol-5- yl)butanoyl]pyrrolidine-2- carboxamide |
| 78 | 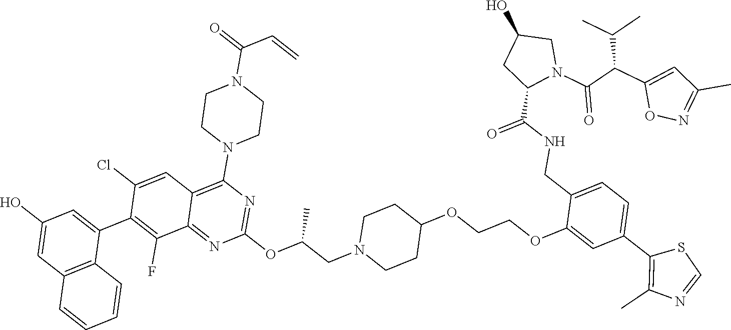 [SMILES: C=CC(=O)N1CCN(c2nc(O[C@H](C)CN3CCC(OCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1] | (2S,4R)-N-({2-[2-({1-[(2R)-2- {[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}oxy)ethoxy]-4-(4-methyl- 1,3-thiazol-5- yl)phenyl}methyl)-4-hydroxy- 1-[(2R)-3-methyl-2-(3-methyl- 1,2-oxazol-5- yl)butanoyl]pyrrolidine-2- carboxamide |
| 79 | 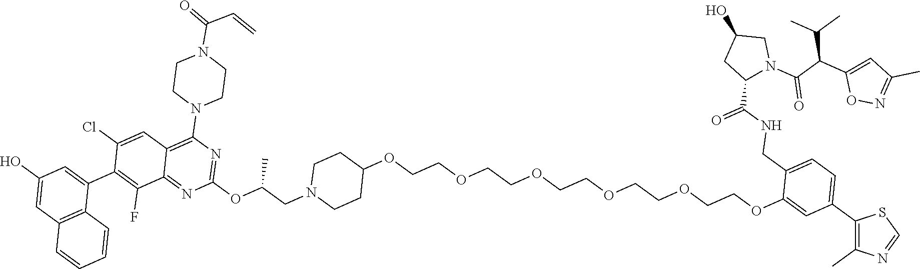 [SMILES: C=CC(=O)N1CCN(c2nc(O[C@H](C)CN3CCC(OCCOCCOCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1] | (2S,4R)-N-[(2-{[14-({1-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}oxy)-3,6,9,12- tetraoxatetradecan-1-yl]oxy}-4- (4-methyl-1,3-thiazol-5- yl)phenyl)methyl]-4-hydroxy- 1-[(2S)-3-methyl-2-(3-methyl- 1,2-oxazol-5- yl)butanoyl]pyrrolidine-2- carboxamide |
| 80 | 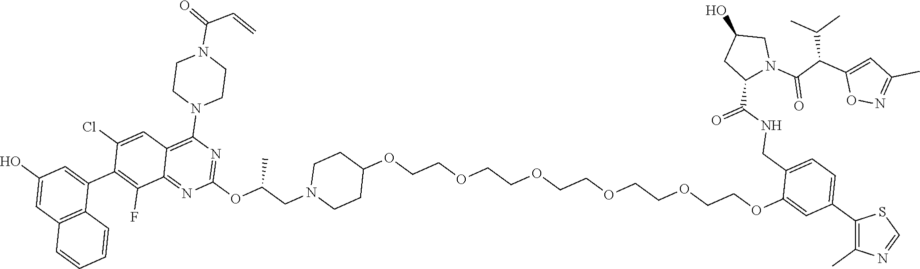 [SMILES: C=CC(=O)N1CCN(c2nc(O[C@H](C)CN3CCC(OCCOCCOCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1] | (2S,4R)-N-[(2-{[14-(1-[(2R)- 2-{[6-chloro-8-fluoro-7-(3- hydroxynaphthalen-1-yl)-4-[4- (prop-2-enoyl)piperazin-1- yl]quinazolin-2- yl]oxy}propyl]piperidin-4- yl}oxy)-3,6,9,12- tetraoxatetradecan-1-yl]oxy}-4- (4-methyl-1,3-thiazol-5- yl)phenyl)methyl]-4-hydroxy- 1-[(2R)-3-methyl-2-(3-methyl- 1,2-oxazol-5- yl)butanoyl]pyrrolidine-2- carboxamide |
| 81 | 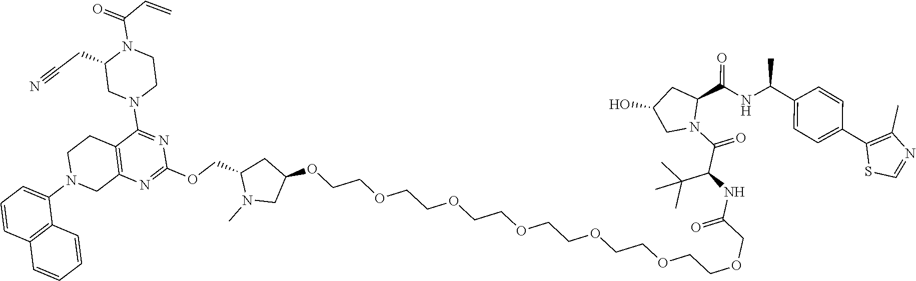 [SMILES: C=CC(=O)N1CCN(c2nc(OC[C@@H]3C[C@@H](OCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CN3C)nc3c2CCN(c2cccc4ccccc24)C3)C[C@@H]1CC#N] | (2S,4R)-1-[(2S)-2-(20- {[(3R,5S)-5-[({4-[(3S)-3- (cyanomethyl)-4-(prop-2- enoyl)piperazin-1-yl]-7- (naphthalen-1-yl)- 5H,6H,7H,8H-pyrido[3,4- d]pyrimidin-2-yl}oxy)methyl]- 1-methylpyrrolidin-3-yl]oxy}- 3,6,9,12,15,18- hexaoxaicosanamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 82 | 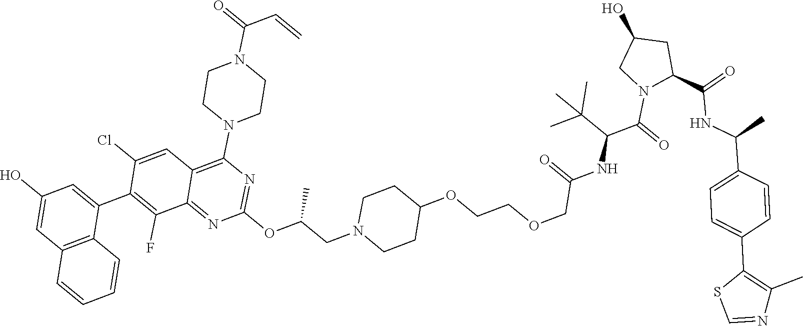 [SMILES: C=CC(=O)N1CCN(c2nc(O[C@H](C)CN3CCC(OCCOCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4S)-1-[(2S)-2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 83 | 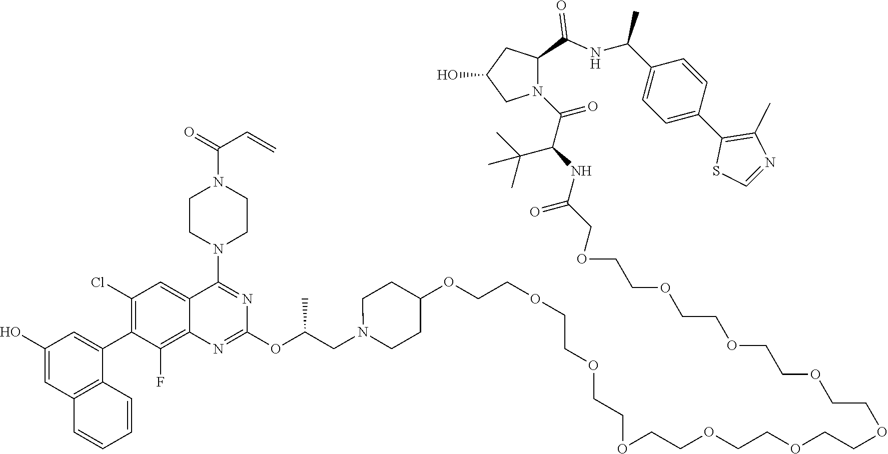 [SMILES: C=CC(=O)N1CCN(c2nc(O[C@H](C)CN3CCC(OCCOCCOCCOCCOCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[32-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)- 3,6,9,12,15,18,21,24,27,30- decaoxadotriacontanamido]- 3,3-dimethylbutanoyl]-4- hydroxy-N-[(1S)-1-[4-(4- methyl-1,3-thiazol-5- yl)phenyl]ethyl]pyrrolidine-2- carboxamide |
| 84 | 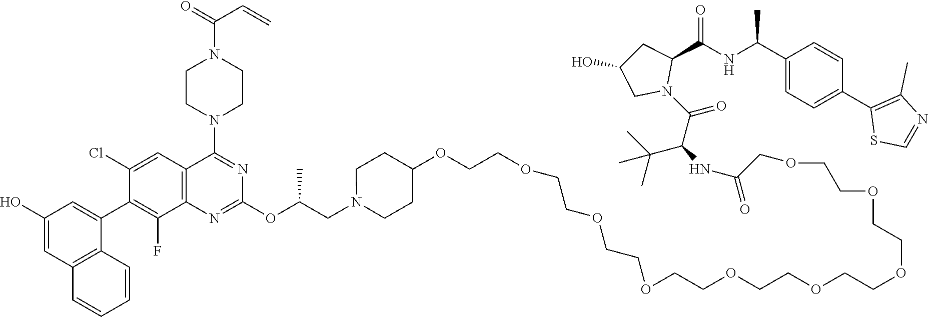 [SMILES: C=CC(=O)N1CCN(c2nc(O[C@H](C)CN3CCC(OCCOCCOCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[26-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)-3,6,9,12,15,18,21,24- octaoxahexacosanamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 85 | 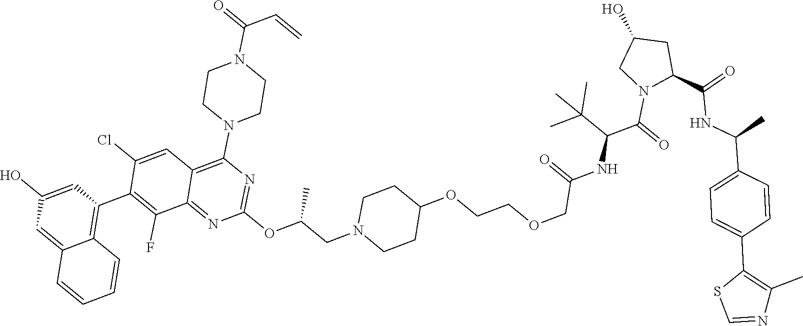 | (2S,4R)-1-[(2S)-2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 86 | 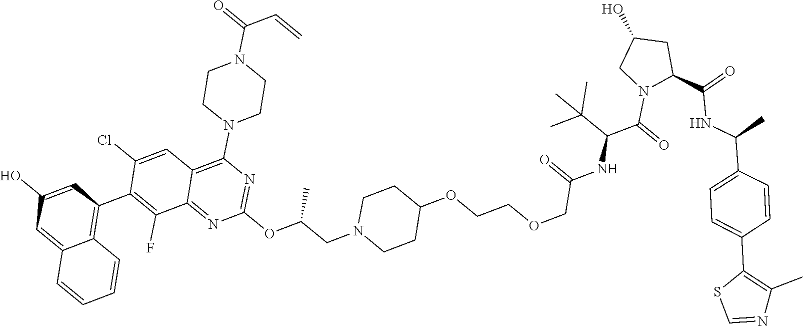 | (2S,4R)-1-[(2S)-2-{2-[2-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 87 | 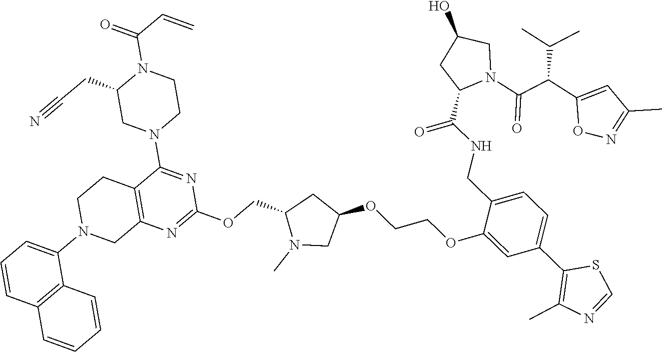 [SMILES: C=CC(=O)N1CCN(c2nc(OC[C@@H]3C[C@@H](OCCOc4cc(-c5scnc5C)ccc4CNC(=O)[C@@H]4C[C@@H](O)CN4C(=O)[C@@H](c4cc(C)no4)C(C)C)CN3C)nc3c2CCN(c2cccc4ccccc24)C3)C[C@@H]1CC#N] | (2S,4R)-N-{[2-(2-{[(3R,5S)-5- [({4-[(3S)-3-(cyanomethyl)-4- (prop-2-enoyl)piperazin-1-yl]- 7-(naphthalen-1-yl)- 5H,6H,7H,8H-pyrido[3,4- d]pyrimidin-2-yl}oxy)methyl]- 1-methylpyrrolidin-3- yl]oxy}ethoxy)-4-(4-methyl- 1,3-thiazol-5- yl)phenyl]methyl}-4-hydroxy- 1-[(2R)-3-methyl-2-(3-methyl- 1,2-oxazol-5- yl)butanoyl]pyrrolidine-2- carboxamide |
| 88 | 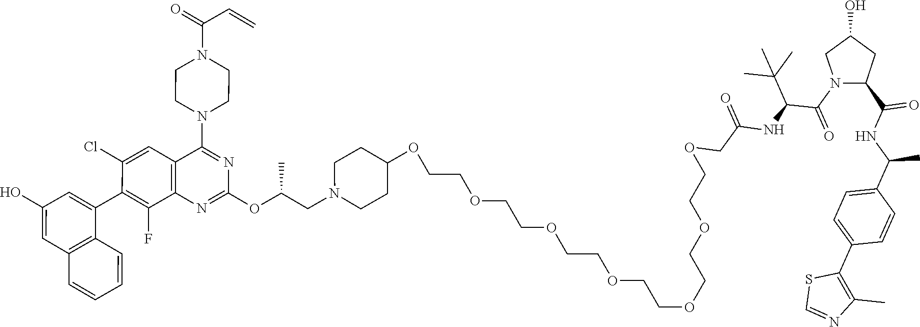 [SMILES: C=CC(=O)N1CCN(c2nc(O[C@H](C)CN3CCC(OCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1] | (2S,4R)-1-[(2S)-2-[20-({1- [(2R)-2-{[6-chloro-8-fluoro-7- (3-hydroxynaphthalen-1-yl)-4- [4-(prop-2-enoyl)piperazin-1- yl]quinazolin-2- yl]oxy}propyl]piperidin-4- yl}oxy)-3,6,9,12,15,18- hexaoxaicosanamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 89 | 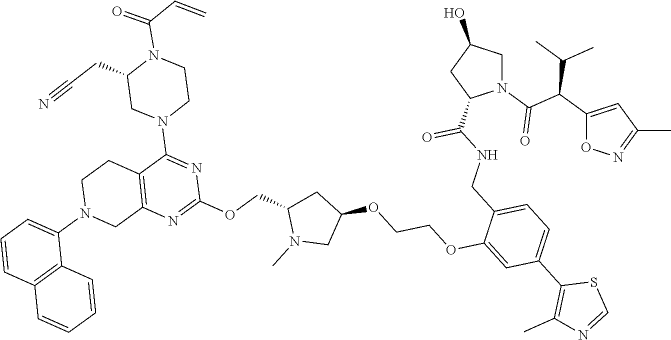 [SMILES: C=CC(=O)N1CCN(c2nc(OC[C@@H]3C[C@@H](OCCOc4cc(-c5scnc5C)ccc4CNC(=O)[C@@H]4C[C@@H](O)CN4C(=O)[C@H](c4cc(C)no4)C(C)C)CN3C)nc3c2CCN(c2cccc4ccccc24)C3)C[C@@H]1CC#N] | (2S,4R)-N-{[2-(2-{[(3R,5S)-5- [({4-[(3S)-3-(cyanomethyl)-4- (prop-2-enoyl)piperazin-1-yl]- 7-(naphthalen-1-yl)- 5H,6H,7H,8H-pyrido[3,4- d]pyrimidin-2-yl}oxy)methyl]- 1-methylpyrrolidin-3- yl]oxy}ethoxy)-4-(4-methyl- 1,3-thiazol-5- yl)phenyl]methyl}-4-hydroxy- 1-[(2S)-3-methyl-2-(3-methyl- 1,2-oxazol-5- yl)butanoyl]pyrrolidine-2- carboxamide |
| 90 | 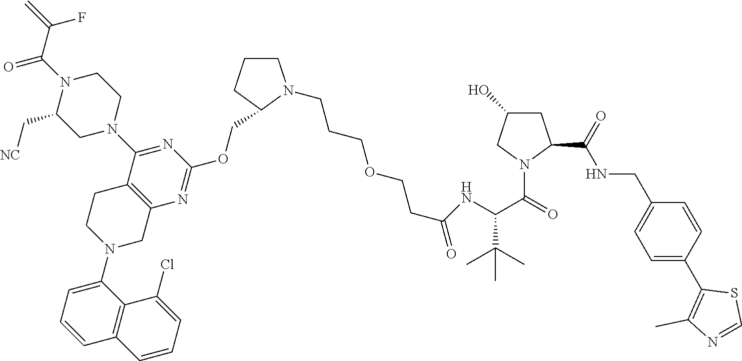 [SMILES: C=C(F)C(=O)N1CCN(c2nc(OC[C@@H]3CCCN3CCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N] | |
| 91 | 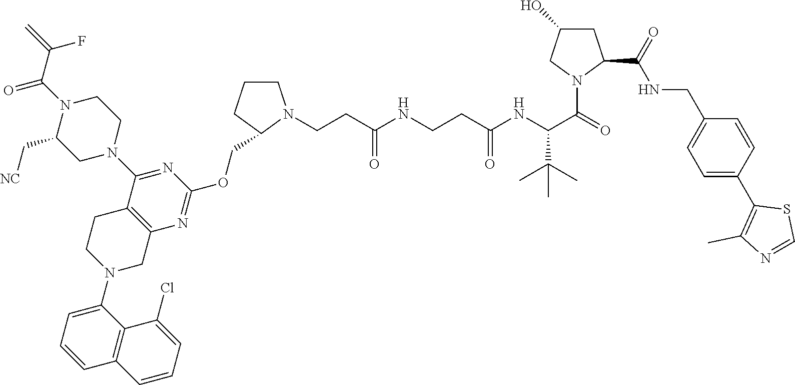 [SMILES: C=C(F)C(=O)N1CCN(c2nc(OC[C@@H]3CCCN3CCC(=O)NCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N] | |
| 92 | 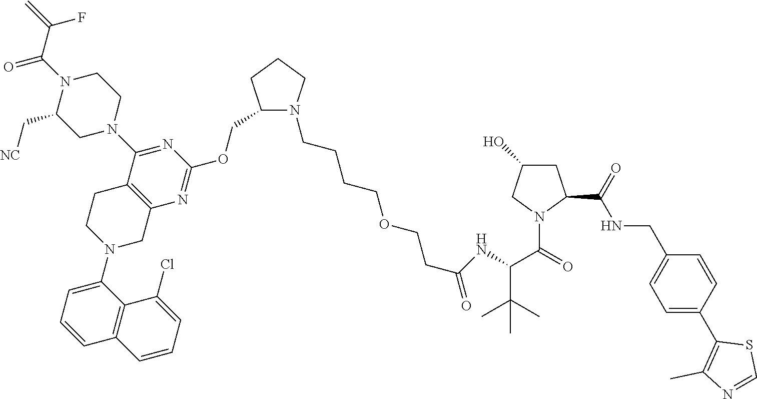 [SMILES: C=C(F)C(=O)N1CCN(c2nc(OC[C@@H]3CCCN3CCCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N] | |
| 93 | 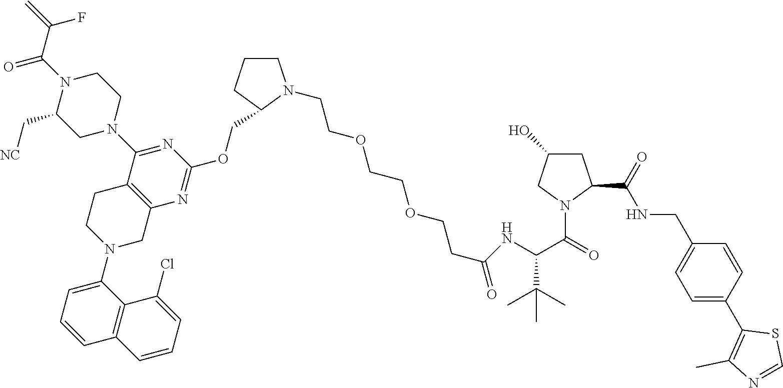 [SMILES: C=C(F)C(=O)N1CCN(c2nc(OC[C@@H]3CCCN3CCOCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N] | |
| 94 | 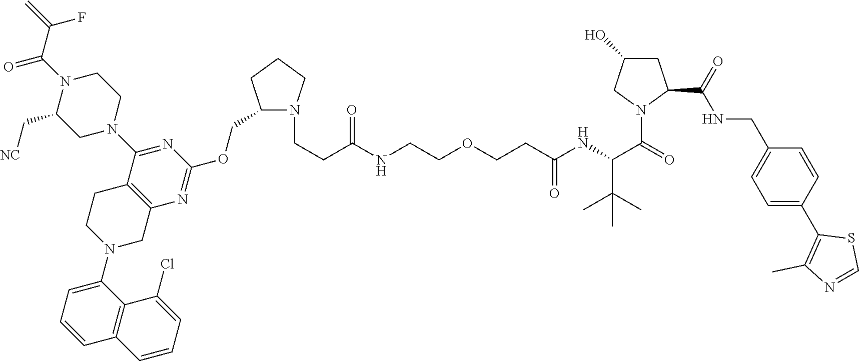 [SMILES: C=C(F)C(=O)N1CCN(c2nc(OC[C@@H]3CCCN3CCC(=O)NCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N] | |
| 95 | 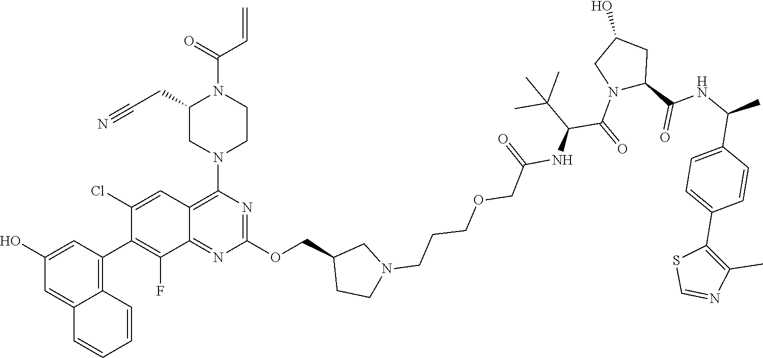 [SMILES: C=CC(=O)N1CCN(c2nc(OC[C@@H]3CCN(CCCOCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)C[C@@H]1CC#N] | (2S,4R)-1-[(2S)-2-(2-{3-[(3R)- 3-[({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)methyl]pyrrolidin-1- yl]propoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 96 | 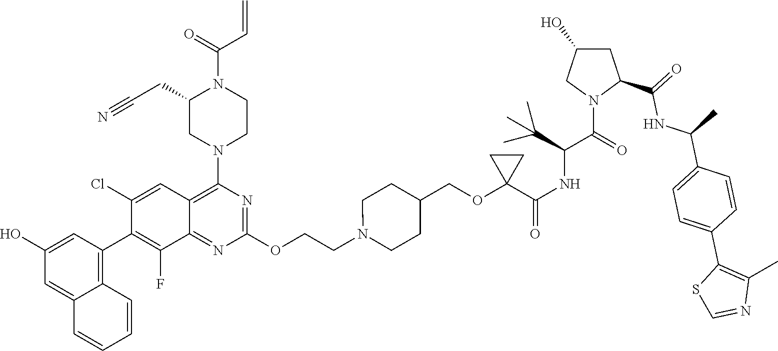 [SMILES: C=CC(=O)N1CCN(c2nc(OCCN3CCC(COC4(C(=O)N[C@H](C(=O)N5C[C@H](O)C[C@H]5C(=O)N[C@@H](C)c5ccc(-c6scnc6C)cc5)C(C)(C)C)CC4)CC3)nc3c(F)c(-c4cc(O)cc5ccccc45)c(Cl)cc23)C[C@@H]1CC#N] | (2S,4R)-1-[(2S)-2-{[1-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)cyclopropyl] formamido}-3,3-dimethylbutanoyl]- 4-hydroxy-N-[(1S)-1-[4-(4- methyl-1,3-thiazol-5- yl)phenyl]ethyl]pyrrolidine-2- carboxamide |
| 97 | 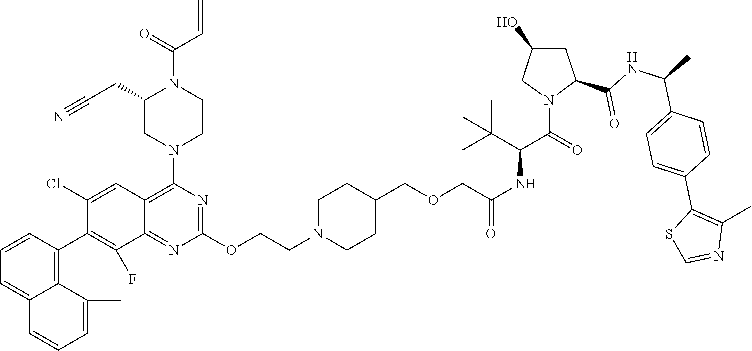 [SMILES: C=CC(=O)N1CCN(c2nc(OCCN3CCC(COCC(=O)N[C@H](C(=O)N4C[C@@H](O)C[C@H]4C(=O)N[C@@H](C)c4ccc(-c5scnc5C)cc4)C(C)(C)C)CC3)nc3c(F)c(-c4cccc5cccc(C)c45)c(Cl)cc23)C[C@@H]1CC#N] | (2S,4S)-1-[(2S)-2-[2-({1-[2- ({6-chloro-4-[(3S)-3- (cyanomethyl)-4-(prop-2- enoyl)piperazin-1-yl]-8-fluoro- 7-(8-methylnaphthalen-1- yl)quinazolin-2- yl}oxy)ethyl]piperidin-4- N-[(1S)-1-[4-(4-methyl-1,3- thiazol-5- yl)phenyl]ethyl]pyrrolidine-2- carboxamide |
| 98 | 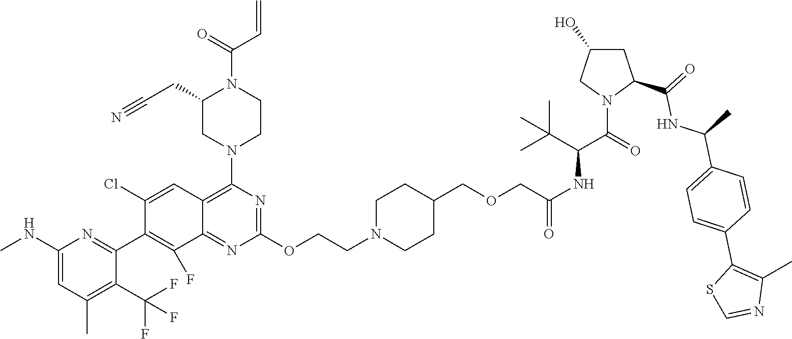 | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-[4-methyl-6-(methylamino)- 3-(trifluoromethyl)pyridin-2- yl]quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 99 | 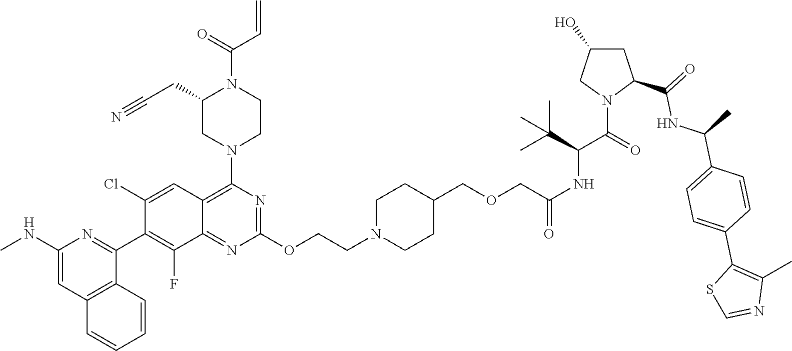 | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-[3- (methylamino)isoquinolin-1- yl]quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 100 | 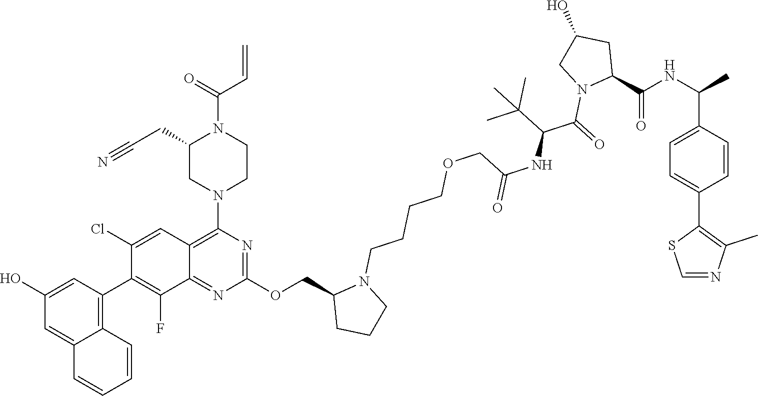 [SMILES: C=CC(=O)N1CCN(c2nc(OC[C@@H]3CCCN3CCCCOCC(=O)N[C@H](C(=O)N3C[C@H](O)C[C@H]3C(=O)N[C@@H](C)c3ccc(-c4scnc4C)cc3)C(C)(C)C)nc3c(F)c(-c4cc(O)cc5ccccc45)c(Cl)cc23)C[C@@H]1CC#N] | (2S,4R)-1-[(2S)-2-(2-{4-[(2S)- 2-[({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)methyl]pyrrolidin-1- yl]butoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 101 | 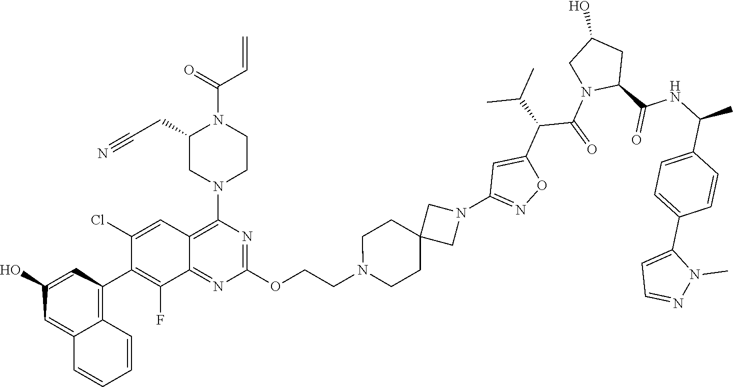 | (2S,4R)-1-[(2S)-2-(3-{7-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(1-methyl-1H- pyrazol-5- yl)phenyl]ethyl]pyrrolidine-2- carboxamide |
| 102 | 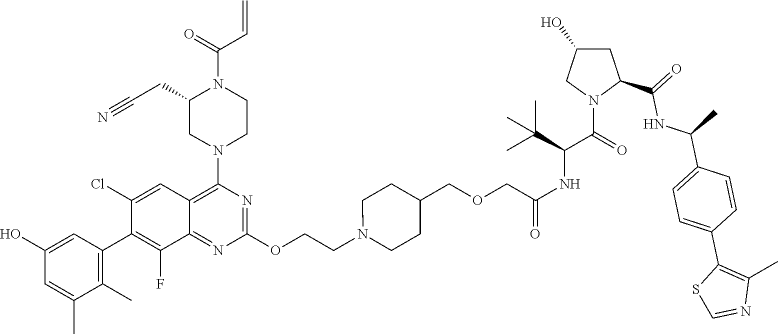 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc(C)c4C)c(Cl)cc23)C[C@@H]1CC#N] | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(5-hydroxy-2,3- dimethylphenyl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 103 | 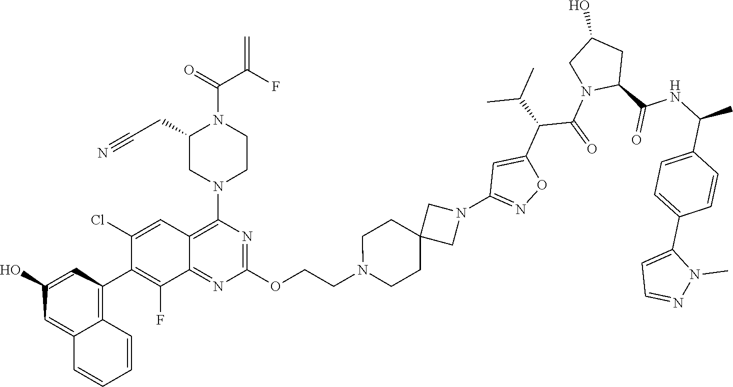 | (2S,4R)-1-[(2S)-2-(3-{7-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(1-methyl-1H- pyrazol-5- yl)phenyl]ethyl]pyrrolidine-2- carboxamide |
| 104 | 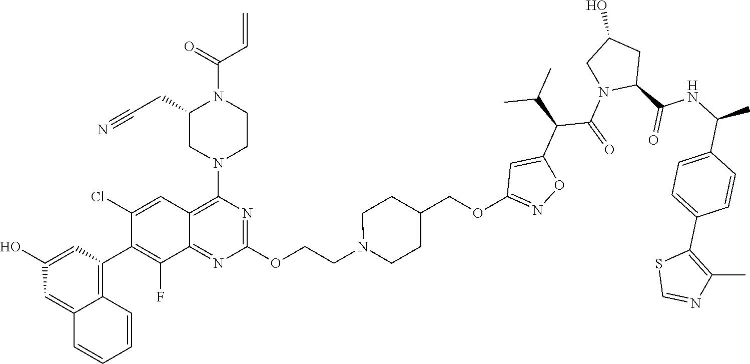 | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 105 | 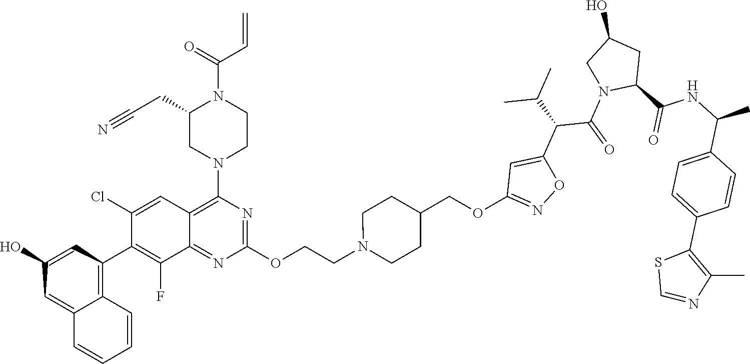 | (2S,4S)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 106 | 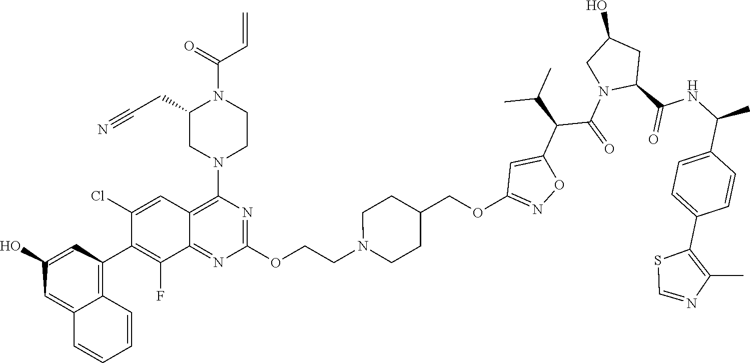 | (2S,4S)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 107 | 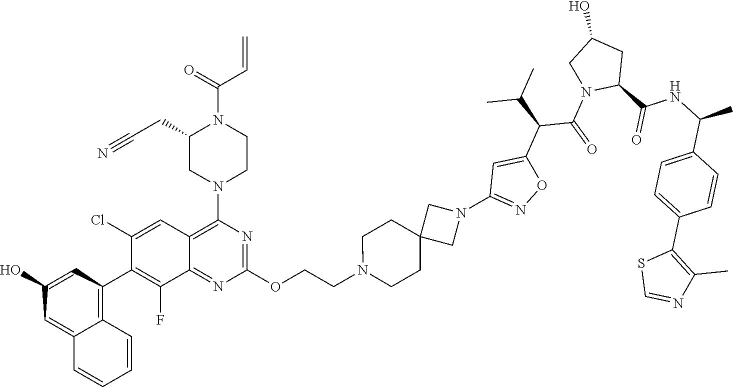 | (2S,4R)-1-[(2R)-2-(3-{7-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(4-methyl-1,3- thiazol-5- yl)phenyl]ethyl]pyrrolidine-2- carboxamide |
| 108 | 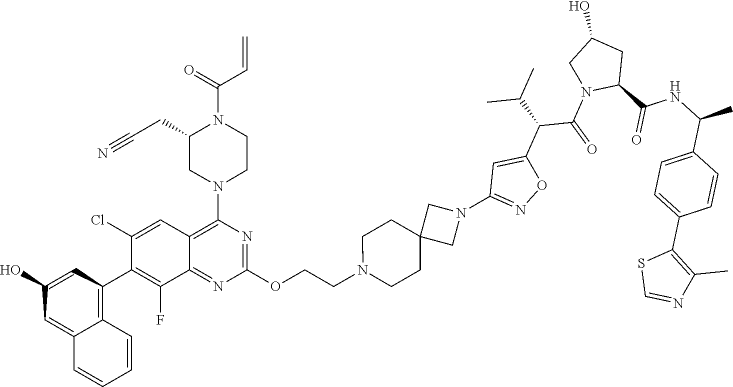 | (2S,4R)-1-[(2S)-2-(3-{7-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(4-methyl-1,3- thiazol-5- yl)phenyl]ethyl]pyrrolidine-2- carboxamide |
| 109 | 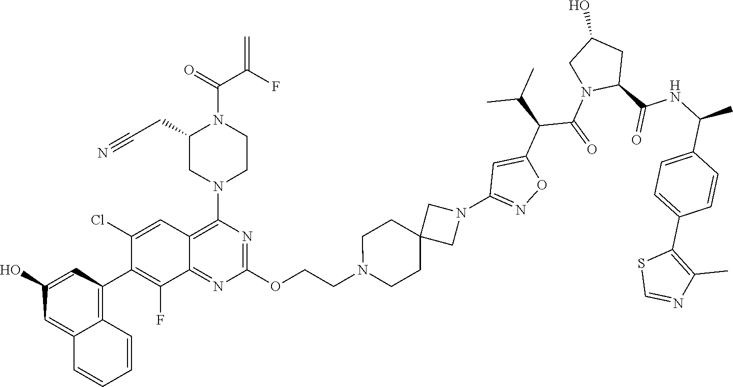 | (2S,4R)-1-[(2R)-2-(3-{7-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(4-methyl-1,3- thiazol-5- yl)phenyl]ethyl]pyrrolidine-2- carboxamide |
| 110 | 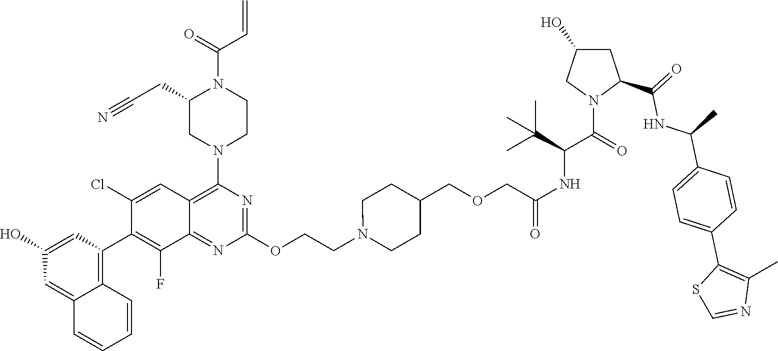 | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 111 | 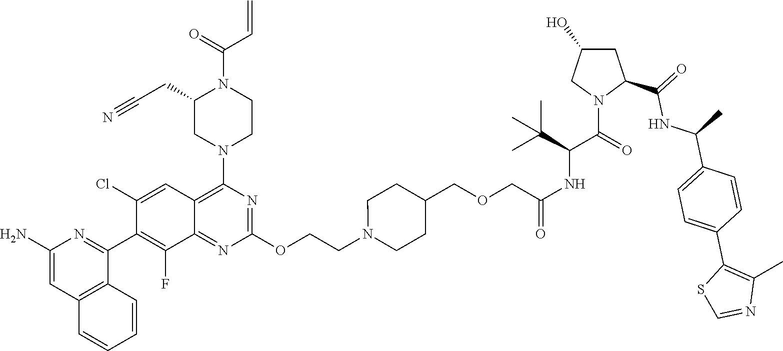 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4nc(N)cc5ccccc45)c(Cl)cc23)C[C@@H]1CC#N] | (2S,4R)-1-(2S)-2-(2-{[1-(2- {[7-(3-aminoisoquinolin-1-yl)- 6-chloro-4-[(3S)-3- (cyanomethyl)-4-(prop-2- enoyl)piperazin-1-yl]-8- fluoroquinazolin-2- yl]oxy}ethyl)piperidin-4- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 112 | 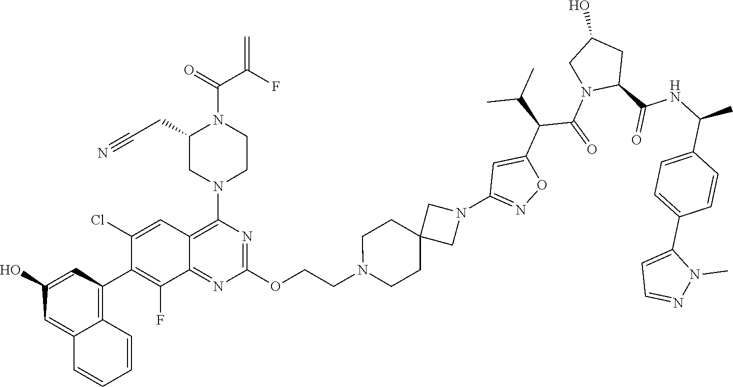 | (2S,4R)-1-[(2R)-2-(3-{7-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(1-methyl-1H- pyrazol-5- yl)phenyl]ethyl]pyrrolidine-2- carboxamide |
| 113 | 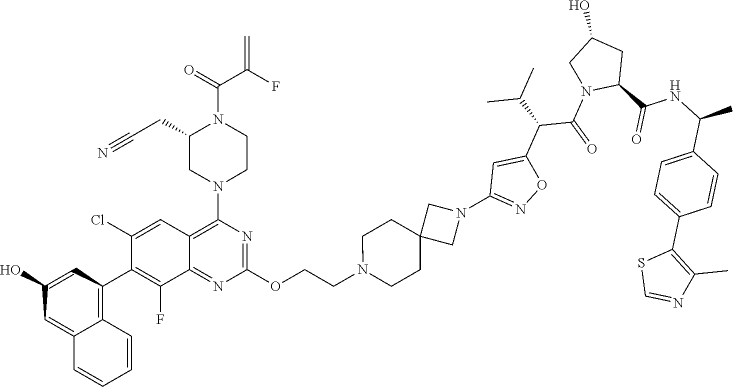 | (2S,4R)-1-[(2S)-2-(3-{7-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(4-methyl-1,3- thiazol-5- yl)phenyl]ethyl]pyrrolidine-2- carboxamide |
| 114 | 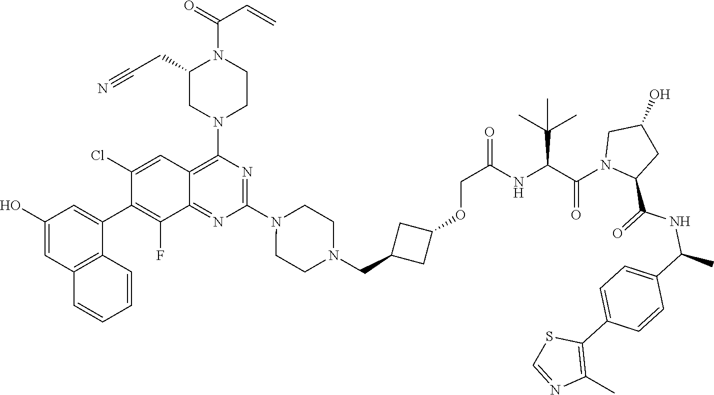 [SMILES: C=CC(=O)N1CCN(c2nc(N3CCN(C[C@H]4C[C@H](OCC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)C[C@@H]1CC#N] | (2S,4R)-1-[(2S)-3,3-dimethyl- 2-{2-[(1r,3r)-3-[(4-{6-chloro-4- [(3S)-3-(cyanomethyl)-4-(prop- 2-enoyl)piperazin-1-yl]-8- fluoro-7-(3- hydroxynaphthalen-1- yl)quinazolin-2-yl}piperazin-1- yl)methyl]cyclobutoxy]acetamido} butanoyl]-4-hydroxy-N- [(1S)-1-[4-(4-methyl-1,3- thiazol-5- yl)phenyl]ethyl]pyrrolidine-2- carboxamide |
| 115 | 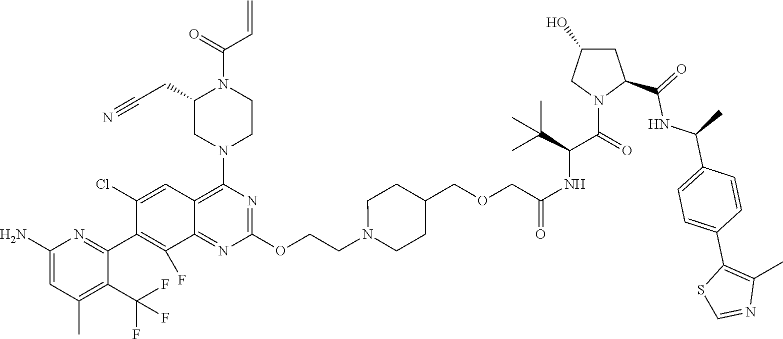 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4nc(N)cc(C)c4C(F)(F)F)c(Cl)cc23)C[C@@H]1CC#N] | (2S,4R)-1-[(2S)-2-[2-({1-[2- ({7-[6-amino-4-methyl-3- (trifluoromethyl)pyridin-2-yl]- 6-chloro-4-[(3S)-3- (cyanomethyl)-4-(prop-2- enoyl)piperazin-1-yl]-8- fluoroquinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 116 | 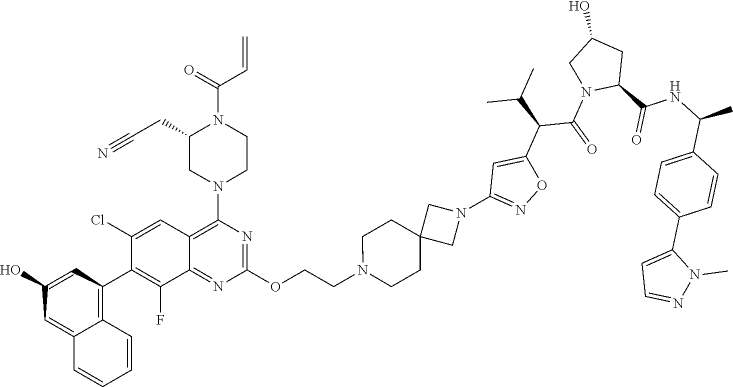 | (2S,4R)-1-[(2R)-2-(3-{7-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(1-methyl-1H- pyrazol-5- yl)phenyl]ethyl]pyrrolidine-2- carboxamide |
| 117 | 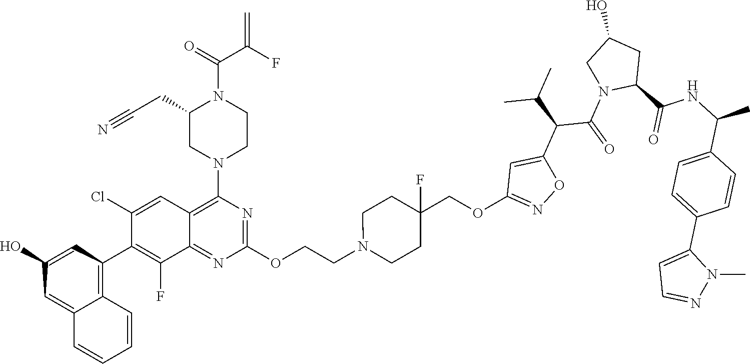 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-fluoropiperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 118 | 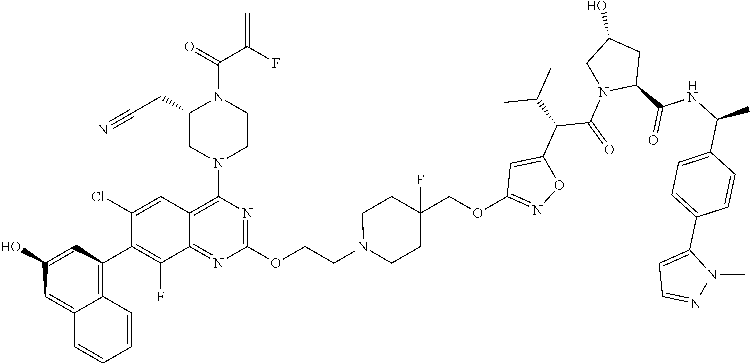 | (2S,4R)-1-[(2S)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-fluoropiperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 119 | 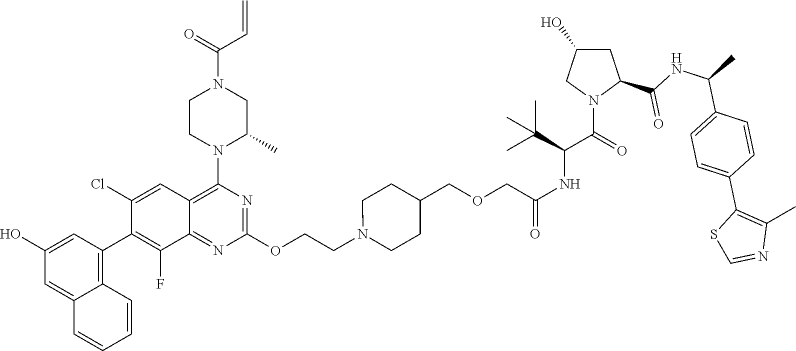 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)[C@@H](C)C1] | (2S,4R)-1-[(2S)-2-(2-{[1-(2- {[6-chloro-8-fluoro-7-(3- hydroxynaphthalen-1-yl)-4- [(2S)-2-methyl-4-(prop-2- enoyl)piperazin-1- yl]quinazolin-2- yl]oxy}ethyl)piperidin-4- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 120 | 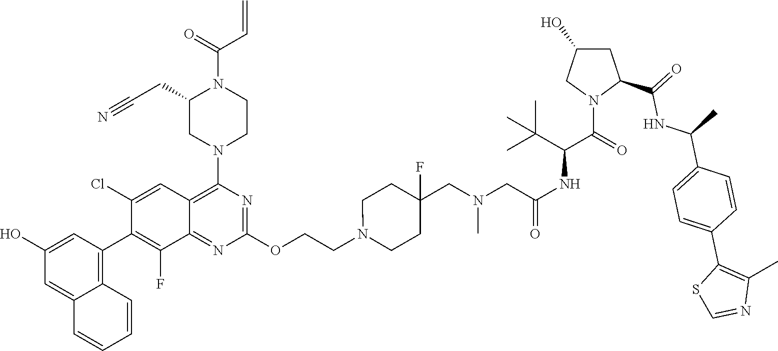 [SMILES: C=CC(=O)N1CCN(c2nc(OCCN3CCC(F)(CN(C)CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-{2-[({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-fluoropiperidin-4- yl}methyl)(methyl)amino] acetamido}-3,3-dimethylbutanoyl]- 4-hydroxy-N-[(1S)-1-[4-(4- methyl-1,3-thiazol-5- yl)phenyl]ethyl]pyrrolidine-2- carboxamide |
| 121 | 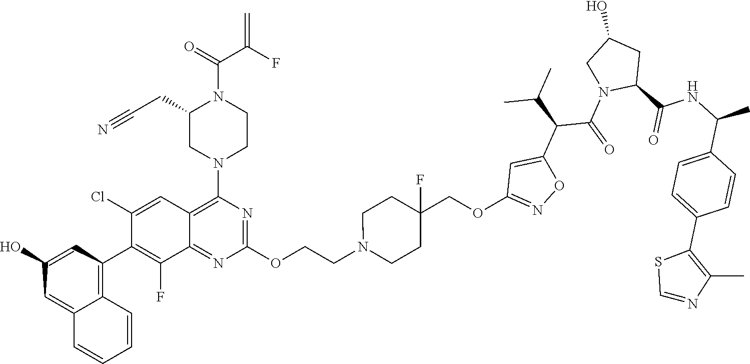 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-fluoropiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 122 | 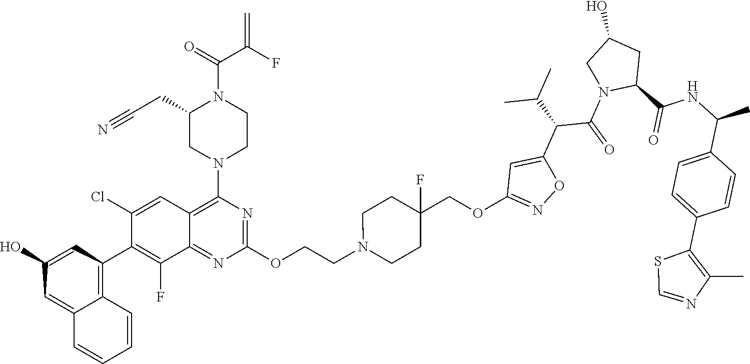 | (2S,4R)-1-[(2S)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-fluoropiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 123 | 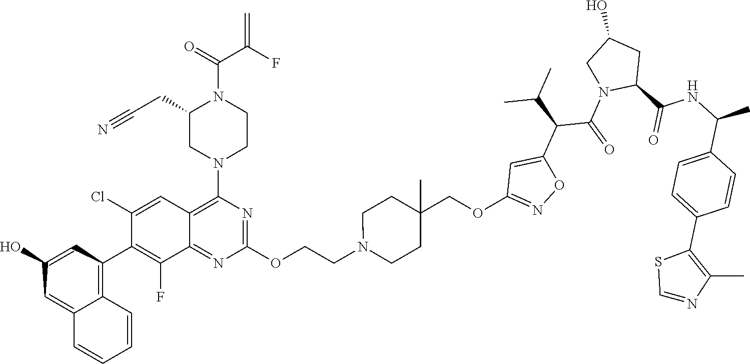 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-methylpiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 124 | 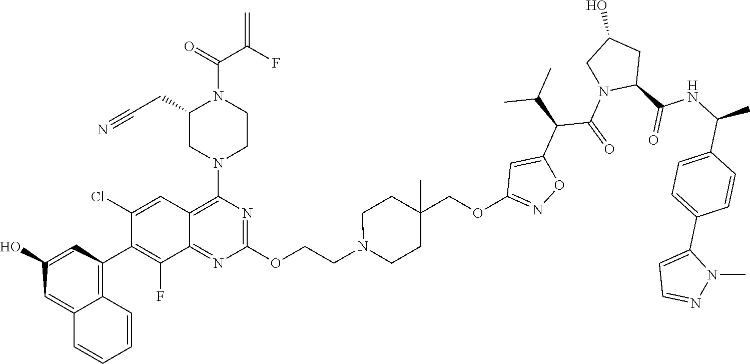 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-methylpiperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 125 | 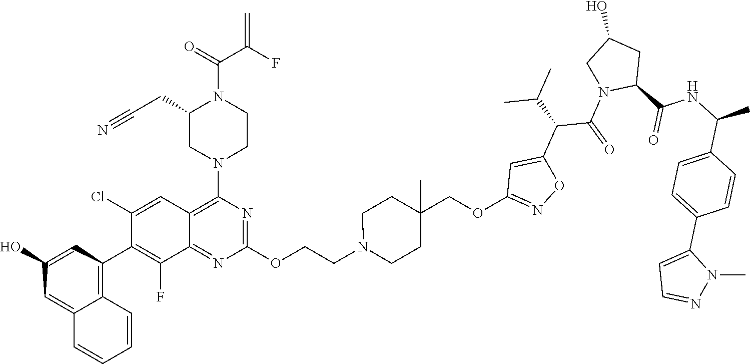 | (2S,4R)-1-[(2S)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-methylpiperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 126 | 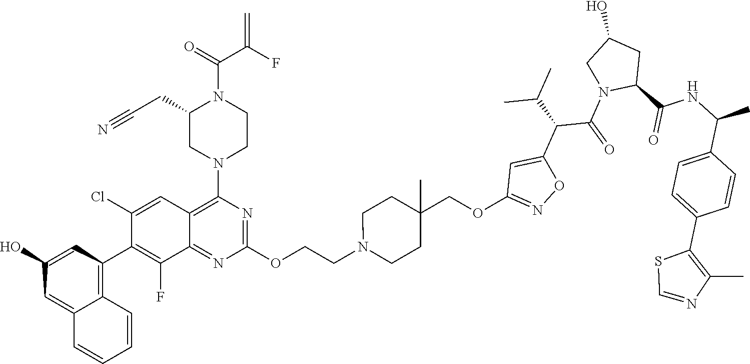 | (2S,4R)-1-[(2S)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2-yl}oxy)ethyl]- 4-methylpiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 127 | 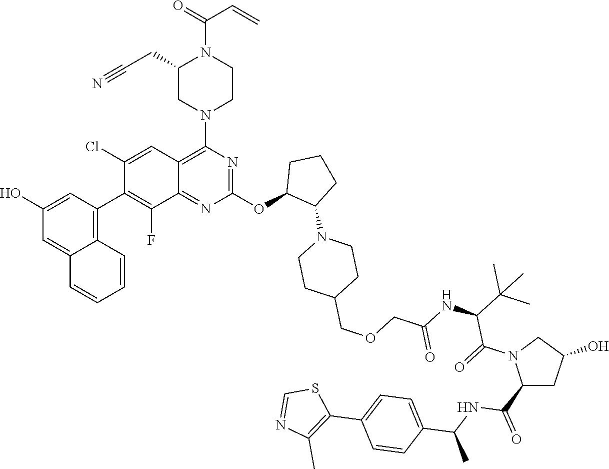 [SMILES: C=CC(=O)N1CCN(c2nc(O[C@H]3CCC[C@@H]3N3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1- [(1S,2S)-2-({6-chloro-4-[(3S)- 3-(cyanomethyl)-4-(prop-2- enoyl)piperazin-1-yl]-8-fluoro- 7-(3-hydroxynaphthalen-1- yl)quinazolin-2- yl}oxy)cyclopentyl]piperidin- 4-yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 128 | 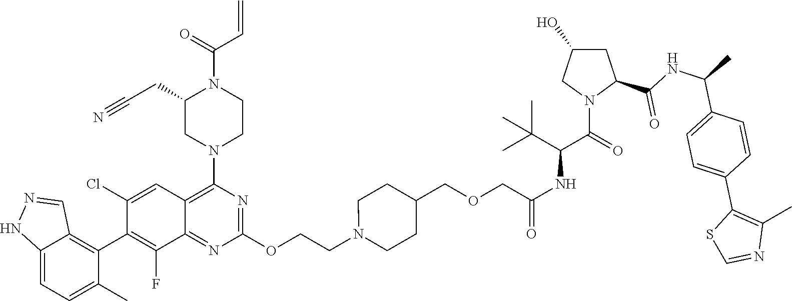 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(C)ccc5[nH]ncc45)c(Cl)cc23)C[C@@H]1CC#N] | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(5-methyl-1H-indazol-4- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 129 | 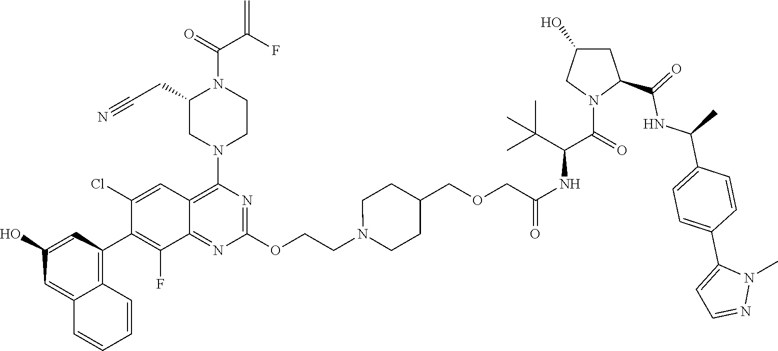 | (2S,4R)-1-[(2S)-2-[2-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(1-methyl-1H- pyrazol-5- yl)phenyl]ethyl]pyrrolidine-2- carboxamide |
| 130 | 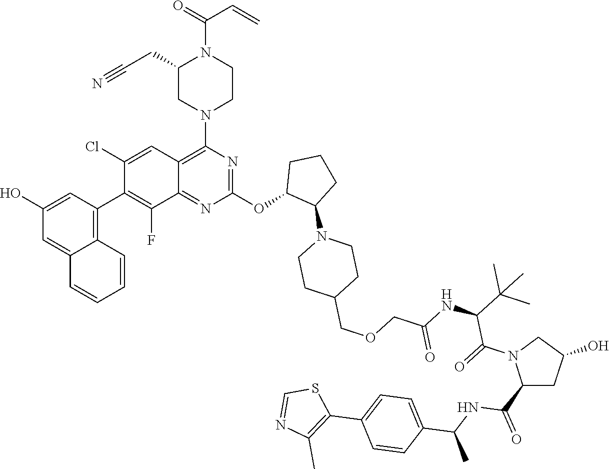 [SMILES: C=CC(=O)N1CCN(c2nc(O[C@@H]3CCC[C@H]3N3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1- [(1R,2R)-2-({6-chloro-4-[(3S)- 3-(cyanomethyl)-4-(prop-2- enoyl)piperazin-1-yl]-8-fluoro- 7-(3-hydroxynaphthalen-1- yl)quinazolin-2- yl}oxy)cyclopentyl]piperidin- 4-yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 131 | 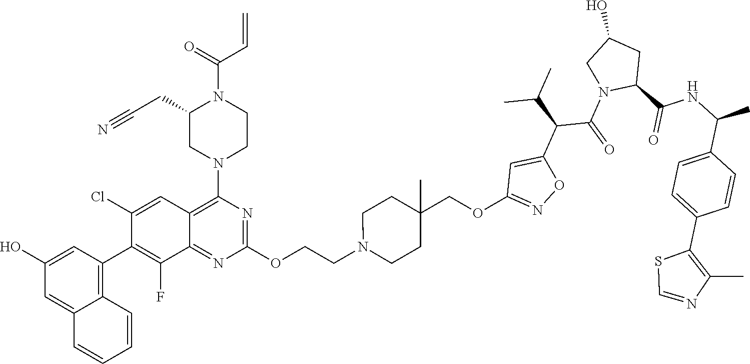 [SMILES: C=CC(=O)N1CCN(c2nc(OCCN3CCC(C)(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-methylpiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 132 | 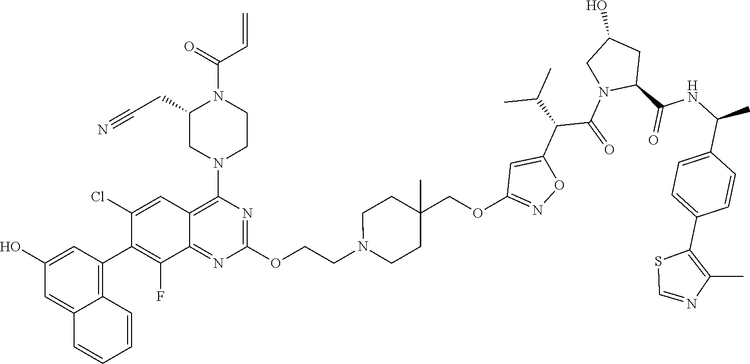 [SMILES: C=CC(=O)N1CCN(c2nc(OCCN3CCC(C)(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-methylpiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 133 | 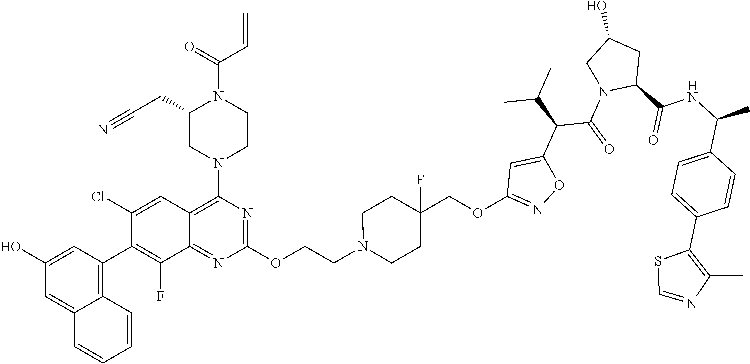 [SMILES: C=CC(=O)N1CCN(c2nc(OCCN3CCC(F)(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-fluoropiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 134 | 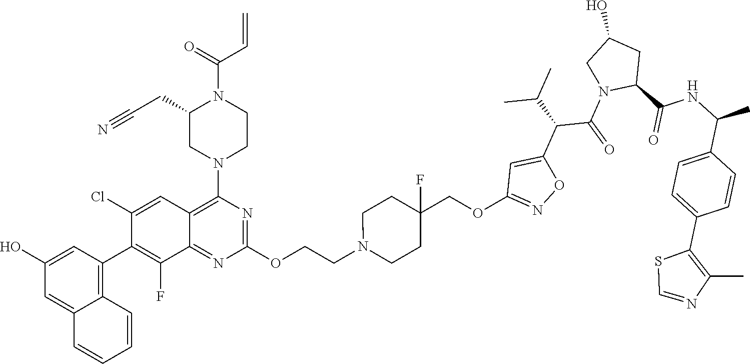 [SMILES: C=CC(=O)N1CCN(c2nc(OCCN3CCC(F)(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-fluoropiperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 135 | 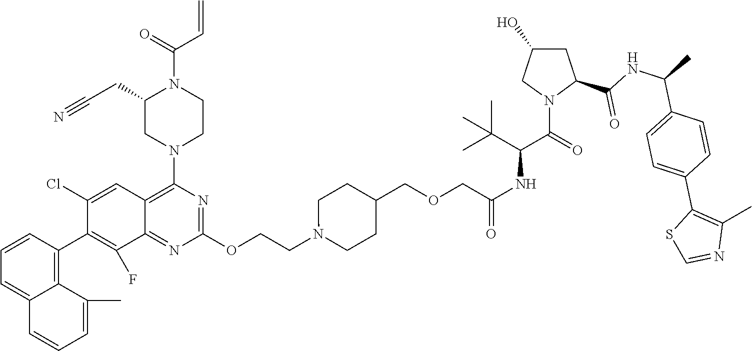 [SMILES: C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cccc5cccc(C)c45)c(Cl)cc23)C[C@@H]1CC#N] | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(8-methylnaphthalen-1- yl)quinazolin-2- yl}oxy)ethyl]-piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 136 | 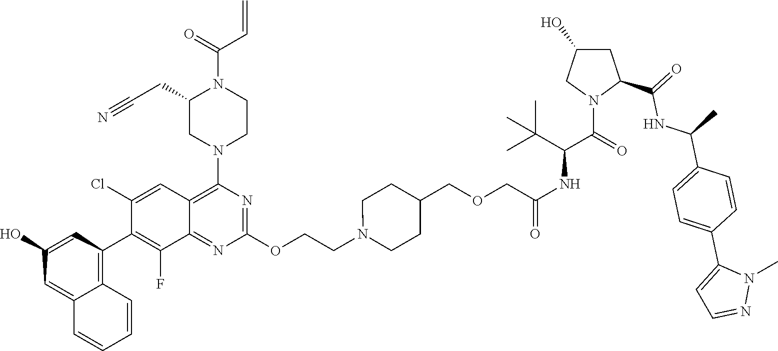 | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(1-methyl-1H- pyrazol-5- yl)phenyl]ethyl]pyrrolidine-2- carboxamide |
| 137 | 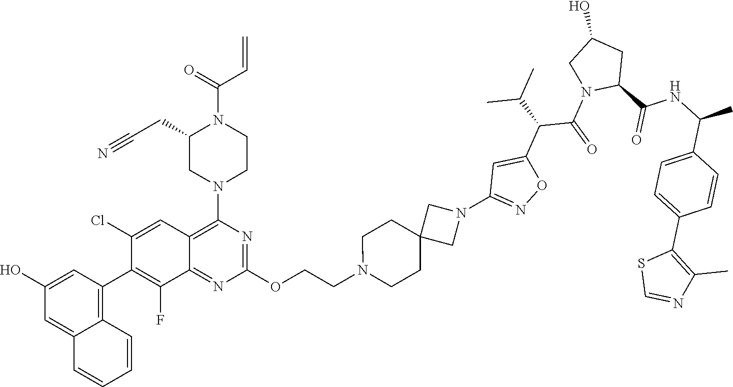 [SMILES: C=CC(=O)N1CCN(c2nc(OCCN3CCC4(CC3)CN(c3cc([C@@H](C(=O)N5C[C@H](O)C[C@H]5C(=O)N[C@@H](C)c5ccc(-c6scnc6C)cc5)C(C)C)on3)C4)nc3c(F)c(-c4cc(O)cc5ccccc45)c(Cl)cc23)C[C@@H]1CC#N] | (2S,4R)-1-[(2S)-2-(3-{7-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(4-methyl-1,3- thiazol-5- yl)phenyl]ethyl]pyrrolidine-2- carboxamide |
| 138 | 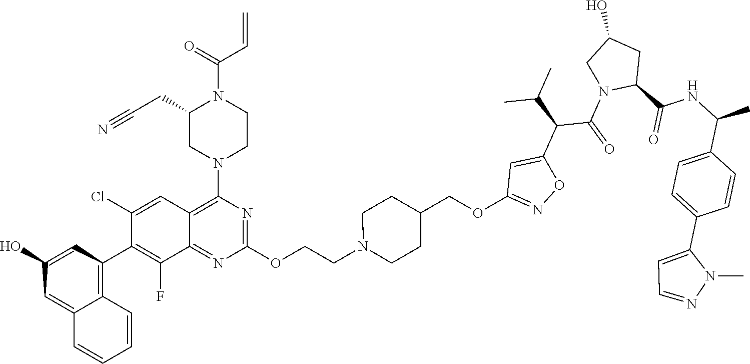 | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]-piperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 139 | 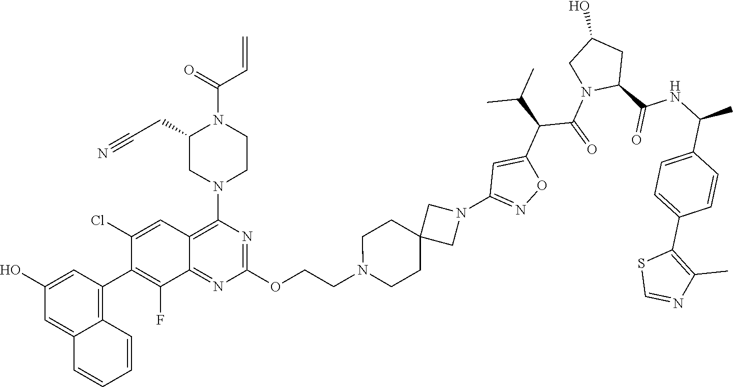 [SMILES: C=CC(=O)N1CCN(c2nc(OCCN3CCC4(CC3)CN(c3cc([C@H](C(=O)N5C[C@H](O)C[C@H]5C(=O)N[C@@H](C)c5ccc(-c6scnc6C)cc5)C(C)C)on3)C4)nc3c(F)c(-c4cc(O)cc5ccccc45)c(Cl)cc23)C[C@@H]1CC#N] | (2S,4R)-1-[(2R)-2-(3-{7-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 2,7-diazaspiro[3.5]nonan-2- yl}-1,2-oxazol-5-yl)-3- methylbutanoyl]-4-hydroxy-N- [(1S)-1-[4-(4-methyl-1,3- thiazol-5- yl)phenyl]ethyl]pyrrolidine-2- carboxamide |
| 140 | 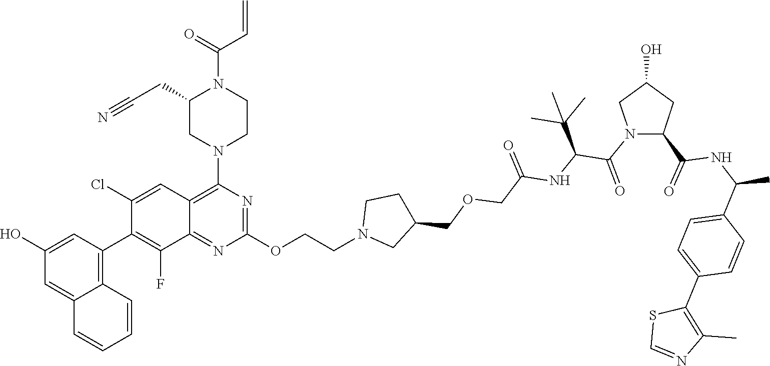 [SMILES: C=CC(=O)N1CCN(c2nc(OCCN3CC[C@@H](COCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)C[C@@H]1CC#N] | (2S,4R)-1-[(2S)-2-(2-{[(3R)-1- [2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]pyrrolidin-3- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 141 | 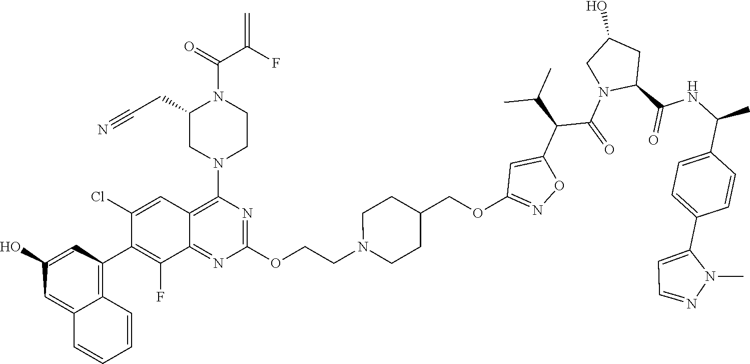 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]-piperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 142 | 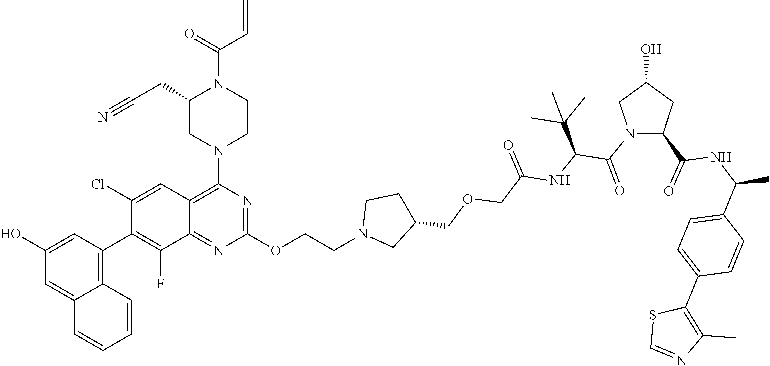 [SMILES: C=CC(=O)N1CCN(c2nc(OCCN3CC[C@H](COCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)C[C@@H]1CC#N] | (2S,4R)-1-[(2S)-2-(2-{[(3S)-1- [2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]pyrrolidin-3- yl]methoxy}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 143 | 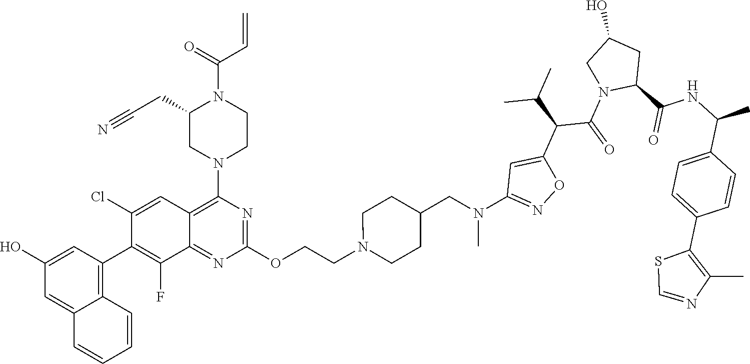 [SMILES: C=CC(=O)N1CCN(c2nc(OCCN3CCC(CN(C)c4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-{3-[({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methyl)(methyl)amino]-1,2- oxazol-5-yl}-3- methylbutanoyl]-4-hydroxy-N- [(1S)-1-[4-(4-methyl-1,3- thiazol-5- yl)phenyl]ethyl]pyrrolidine-2- carboxamide |
| 144 | 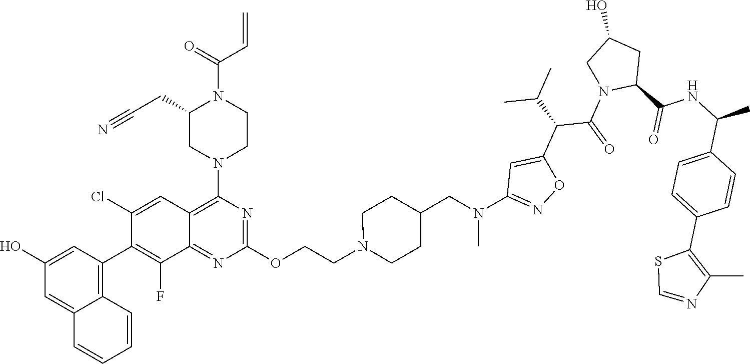 [SMILES: C=CC(=O)N1CCN(c2nc(OCCN3CCC(CN(C)c4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-{3-[({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methyl)(methyl)amino]-1,2- oxazol-5-yl}-3- methylbutanoyl]-4-hydroxy-N- [(1S)-1-[4-(4-methyl-1,3- thiazol-5- yl)phenyl]ethyl]pyrrolidine-2- carboxamide |
| 145 | 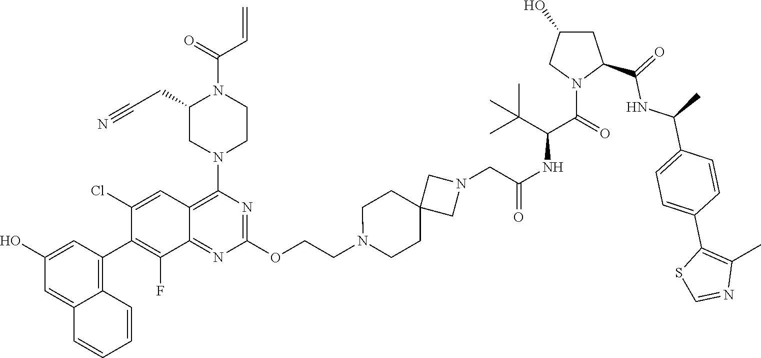 [SMILES: C=CC(=O)N1CCN(c2nc(OCCN3CCC4(CC3)CN(CC(=O)N[C@H](C(=O)N3C[C@H](O)C[C@H]3C(=O)N[C@@H](C)c3ccc(-c5scnc5C)cc3)C(C)(C)C)C4)nc3c(F)c(-c4cc(O)cc5ccccc45)c(Cl)cc23)C[C@@H]1CC#N] | (2S,4R)-1-[(2S)-2-(2-{7-[2- ({6-chloro-4-[(3S)-3- (cyanomethyl)-4-(prop-2- enoyl)piperazin-1-yl)-8-fluoro- 7-(3-hydroxynaphthalen-1- yl)quinazolin-2-yl}oxy)ethyl]- 2,7-diazaspiro[3.5]nonan-2- yl}acetamido)-3,3- dimethylbutanoyl]-4-hydroxy- N-[(1S)-1-[4-(4-methyl-1,3- thiazol-5- yl)phenyl]ethyl]pyrrolidine-2- carboxamide |
| 146 | 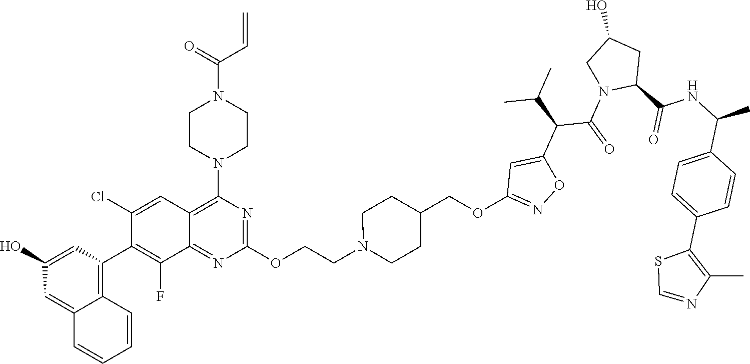 | (2S,4R)-1-[(2R)-2-(3-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]methoxy}-1,2-oxazol-5-yl)- 3-methylbutanoyl]-4-hydroxy- N-[(1S)-1-[4-(4-methyl-1,3- thiazol-5- yl)phenyl]ethyl]pyrrolidine-2- carboxamide |
| 147 | 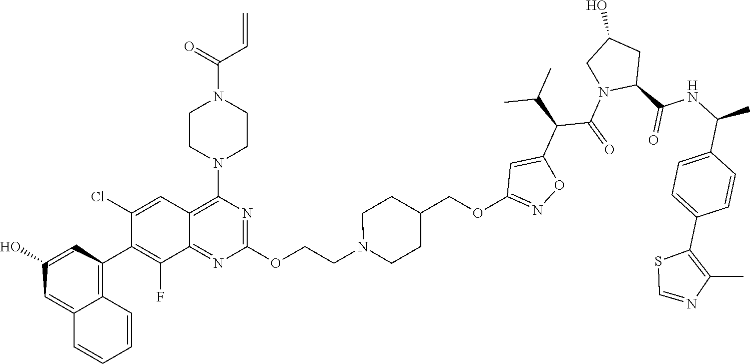 | (2S,4R)-1-[(2R)-2-(3-{[1-(2- {[6-chloro-8-fluoro-7-(3- hydroxynaphthalen-1-yl)-4-[4- (prop-2-enoyl)piperazin-1- yl]quinazolin-2- yl]oxy}ethyl)piperidin-4- yl]methoxy}-1,2-oxazol-5-yl)- 3-methylbutanoyl]-4-hydroxy- N-[(1S)-1-[4-(4-methyl-1,3- thiazol-5- yl)phenyl]ethyl]pyrrolidine-2- carboxamide |
| 148 | 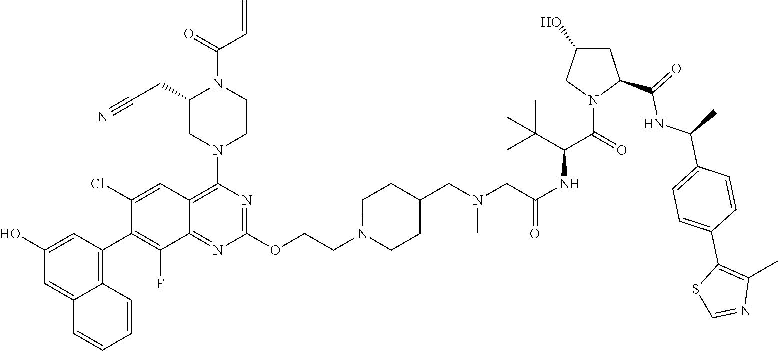 [SMILES: C=CC(=O)N1CCN(c2nc(OCCN3CCC(CN(C)CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-{2-[({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4-yl} methyl)(methyl)amino] acetamido}-3,3- dimethylbutanoyl]- 4-hydroxy-N-[(1S)-1-[4-(4- methyl-1,3-thiazol-5- yl)phenyl]ethyl]pyrrolidine-2- carboxamide |
| 149 | 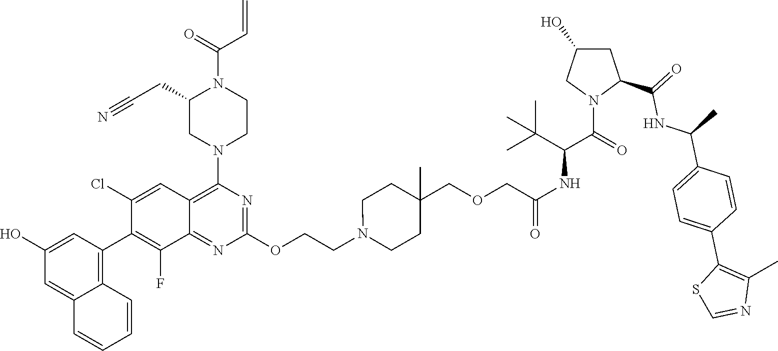 [SMILES: C=CC(=O)N1CCN(c2nc(OCCN3CCC(C)(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-methylpiperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 150 | 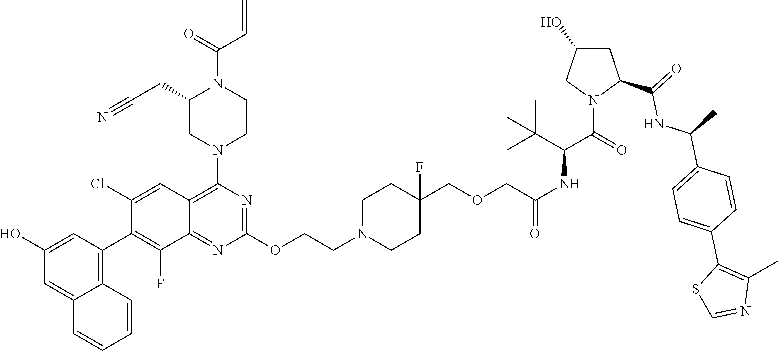 [SMILES: C=CC(=O)N1CCN(c2nc(OCCN3CCC(F)(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2-yl}oxy)ethyl]- 4-fluoropiperidin-4- yl}methoxy)acctamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 151 | 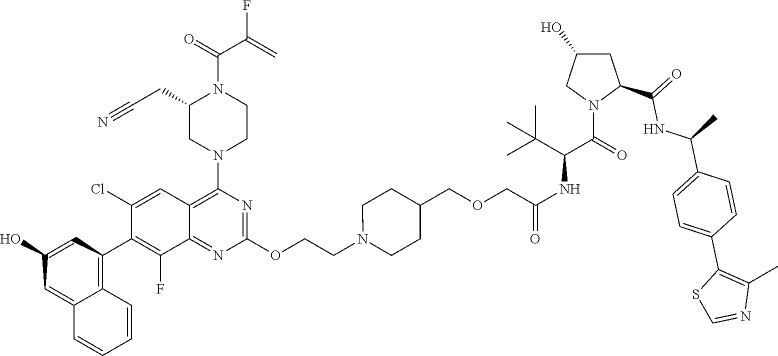 | (2S,4R)-1-[(2S)-2-[2-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 152 | 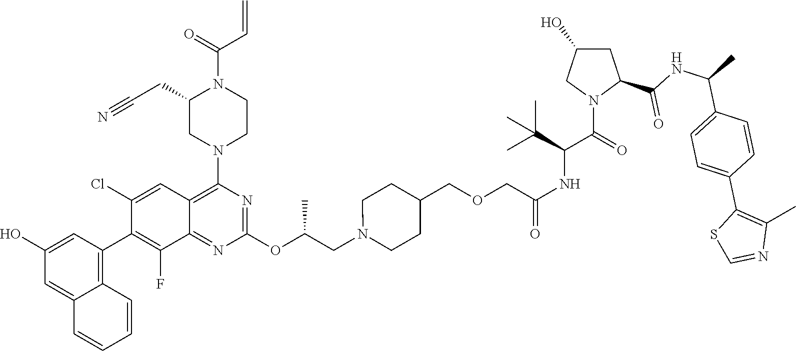 [SMILES: C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-[2-({1- [(2R)-2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)propyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 153 | 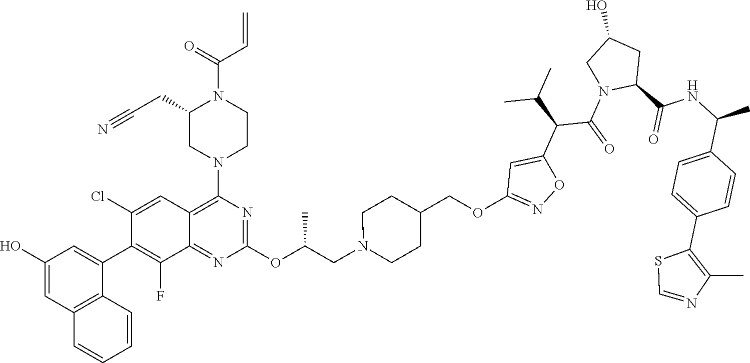 [SMILES: C=CC(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1- [(2R)-2-({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)propyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 154 | 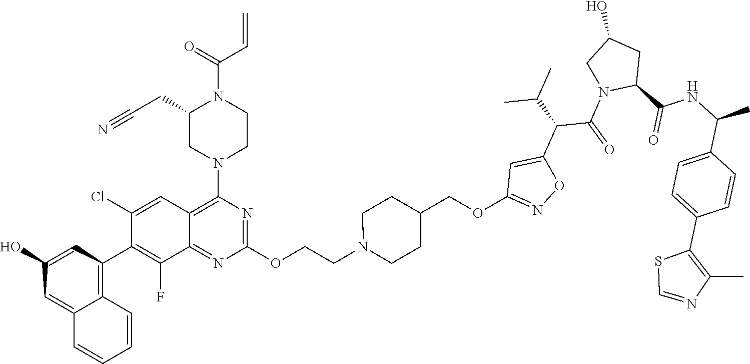 | (2S,4R)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 155 | 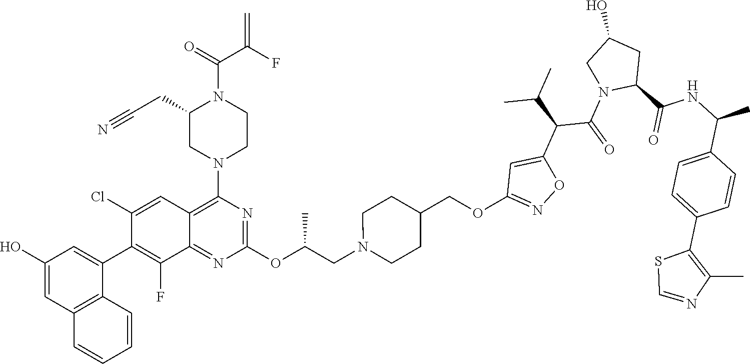 [SMILES: C=C(F)C(=O)N1CCN(c2nc(O[C@H](C)CN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1- [(2R)-2-({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)propyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 156 | 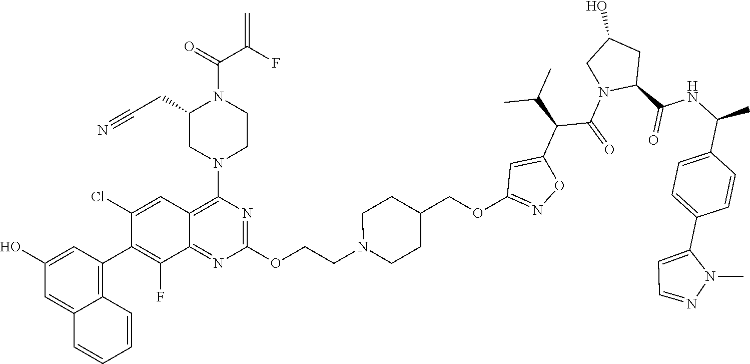 [SMILES: C=C(F)C(=O)N1CCN(c2nc(OCCN3CCC(COc4cc([C@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 157 | 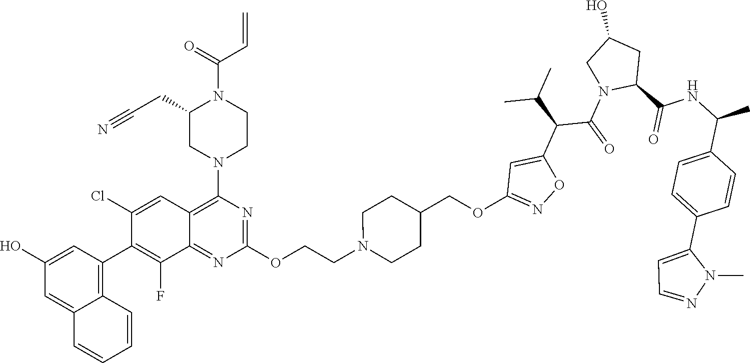 [SMILES: C=CC(=O)N1CCN(c2nc(OCCN3CCC(COc4cc([C@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 158 | 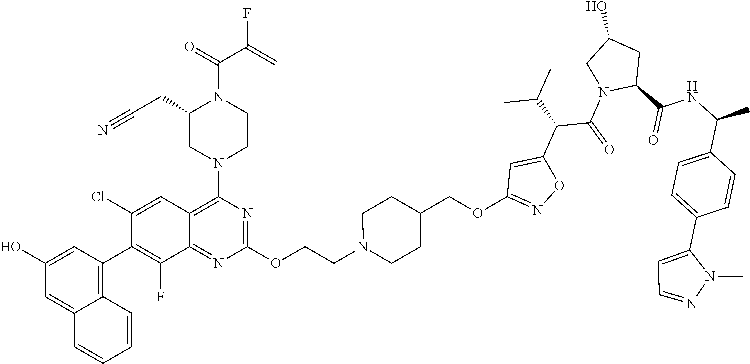 [SMILES: C=C(F)C(=O)N1CCN(c2nc(OCCN3CCC(COc4cc([C@@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 159 | 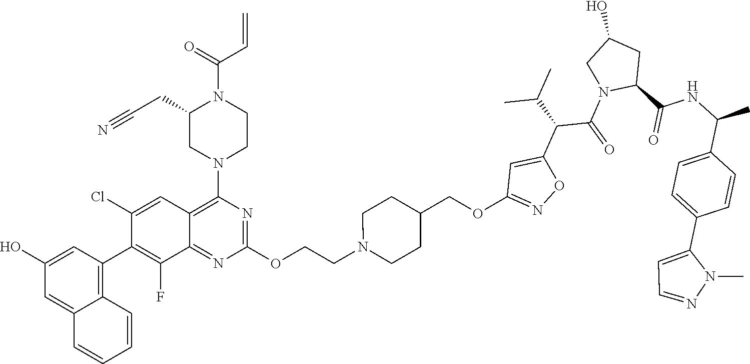 [SMILES: C=CC(=O)N1CCN(c2nc(OCCN3CCC(COc4cc([C@@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(1-methyl-1H- pyrazol-5- yl)phenyl]ethyl]pyrrolidine-2- carboxamide |
| 160 | 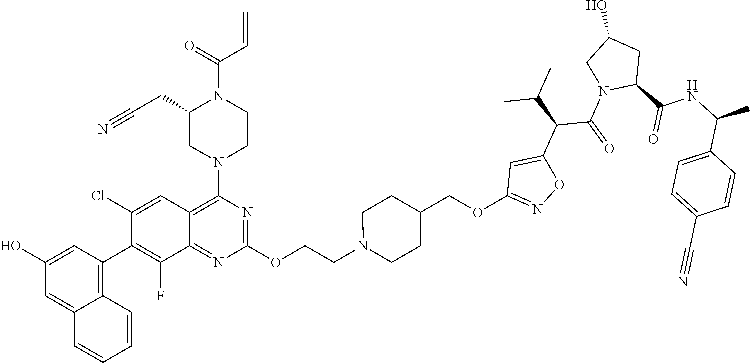 [SMILES: C=CC(=O)N1CCN(c2nc(OCCN3CCC(COc4cc([C@H](C(=O)N5C[C@H](O)C[C@H]5C(=O)N[C@@H](C)c5ccc(C#N)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-N-[(1S)-1- (4-cyanophenyl)ethyl]-4- hydroxypyrrolidine-2- carboxamide |
| 161 | 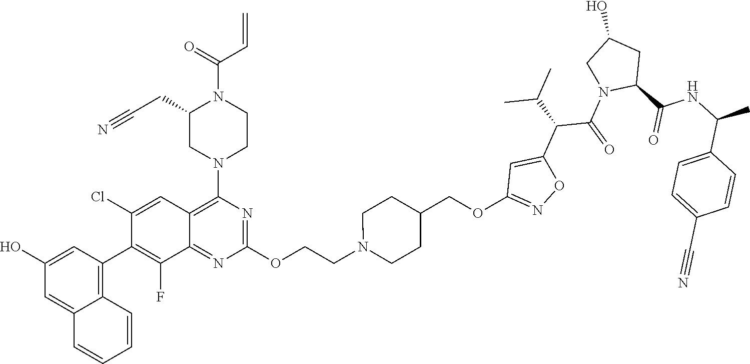 [SMILES: C=CC(=O)N1CCN(c2nc(OCCN3CCC(COc4cc([C@@H](C(=O)N5C[C@H](O)C[C@H]5C(=O)N[C@@H](C)c5ccc(C#N)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-N-[(1S)-1- (4-cyanophenyl)ethyl]-4- hydroxypyrrolidine-2- carboxamide |
| 162 | 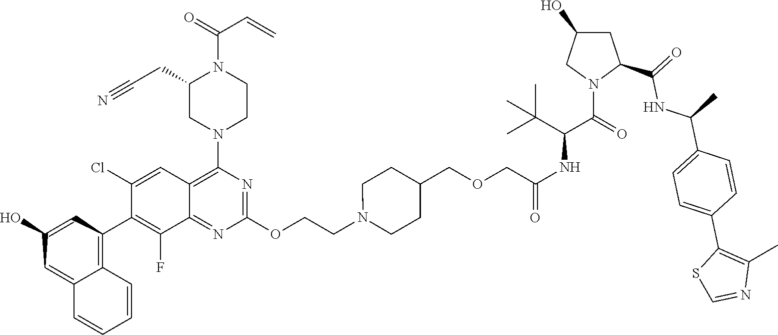 | (2S,4S)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 163 | 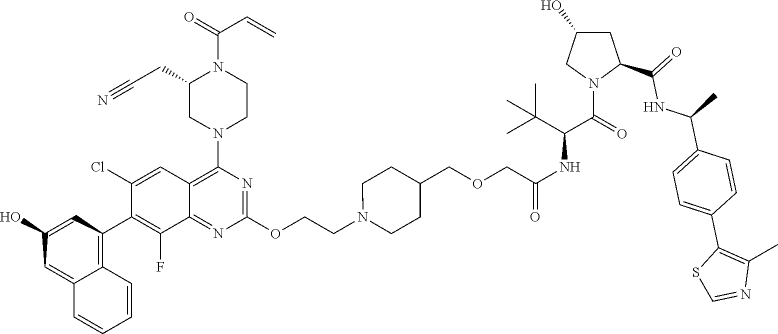 | (2S,4R)-1-[(2S)-2-[2-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 164 | 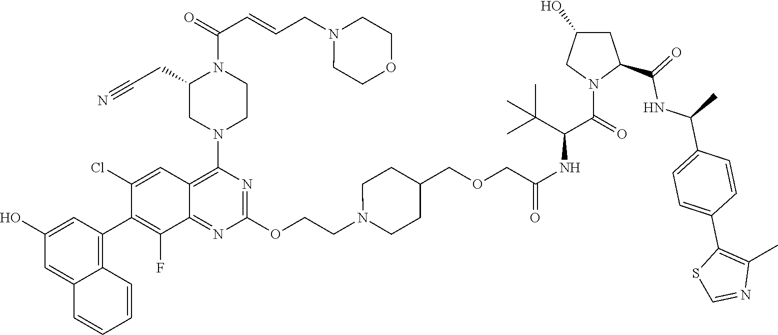 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCN(C(=O)/C=C/CN5CCOCC5)[C@@H](CC#N)C4)c4cc(Cl)c(-c5cc(O)cc6ccccc56)c(F)c4n3)CC2)C(C)(C)C)cc1] | (2S,4R)-1-[(2S)-2-[2-({1-[2- ({6-chloro-4-[(3S)-3- (cyanomethyl)-4-[(2E)-4- (morpholin-4-yl)but-2- enoyl]piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 165 | 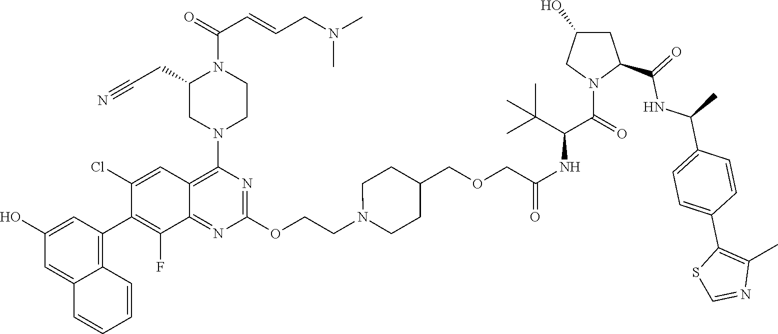 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CCN(C(=O)/C=C/CN(C)C)[C@@H](CC#N)C4)c4cc(Cl)c(-c5cc(O)cc6ccccc56)c(F)c4n3)CC2)C(C)(C)C)cc1] | (2S,4R)-1-[(2S)-2-[2-({1-[2- ({6-chloro-4-[(3S)-3- (cyanomethyl)-4-[(2E)-4- (dimethylamino)but-2- enoyl]piperazin-1-yl]-8-fluoro- 7-(3-hydroxynaphthalen-1- yl)quinazolin-2- yl}oxy)ethyl]piperidin-4- yl}methoxy)acetamido]-3,3- dimethylbutanoyl]-4-hydroxy- N-[(1S)-1-[4-(4-methyl-1,3- thiazol-5- yl)phenyl]ethyl]pyrrolidine-2- carboxamide |
| 166 | 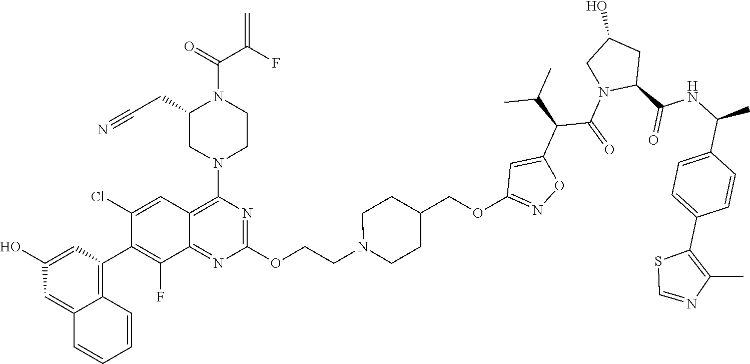 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 167 | 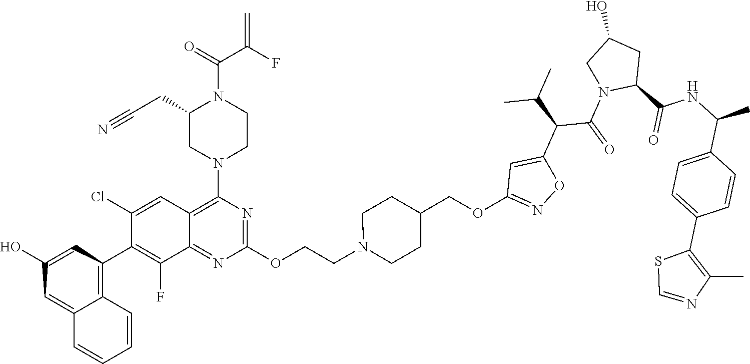 | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 168 | 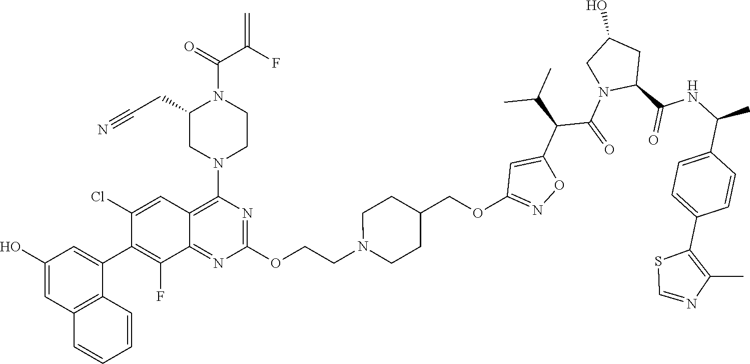 [SMILES: C=C(F)C(=O)N1CCN(c2nc(OCCN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 169 | 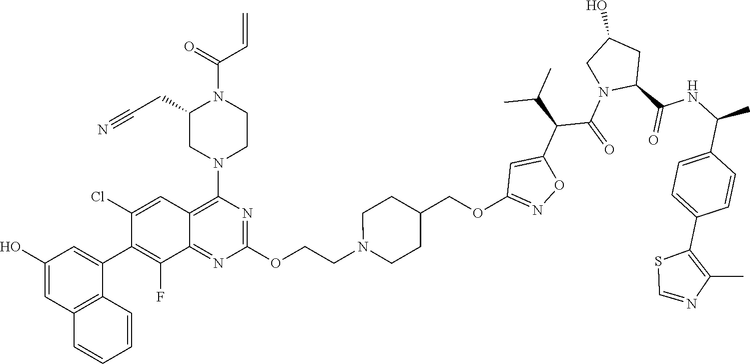 [SMILES: C=CC(=O)N1CCN(c2nc(OCCN3CCC(COc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 170 | 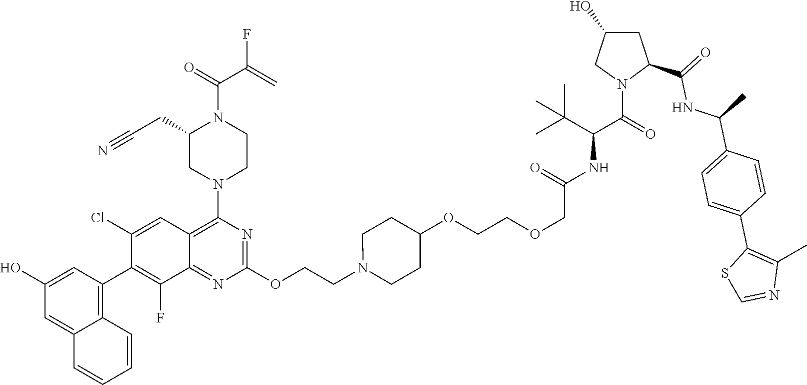 [SMILES: C=C(F)C(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N] | (2S,4R)-1-[(2S)-2-{2-[2-({1- [2-({6-chloro-4-[(3S)-3- (cyanomethyl)-4-(2- fluoroprop-2-enoyl)piperazin- 1-yl]-8-fluoro-7-(3- hydroxynaphthalen-1- yl)quinazolin-2- yl}oxy)ethyl]piperidin-4- yl}oxy)ethoxy]acetamido}-3,3- dimethylbutanoyl]-4-hydroxy- N-[(1S)-1-[4-(4-methyl-1,3- thiazol-5- yl)phenyl]ethyl]pyrrolidine-2- carboxamide |
| 171 | 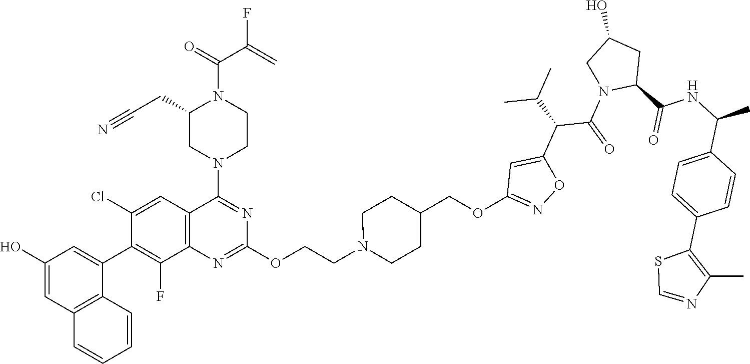 [SMILES: C=C(F)C(=O)N1CCN(c2nc(OCCN3CCC(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(2- fluoroprop-2-enoyl)piperazin- l-yl]-8-fluoro-7-(3- hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 172 | 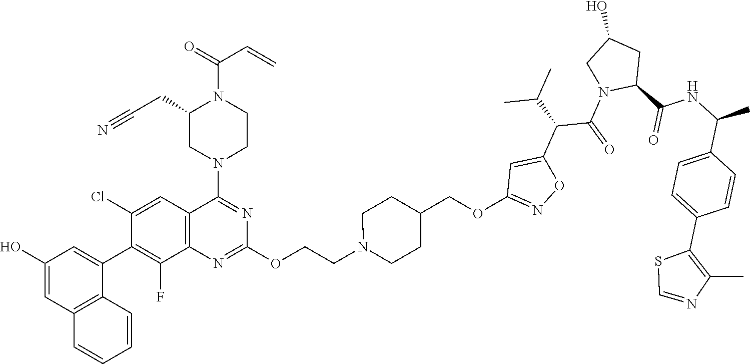 [SMILES: C=CC(=O)N1CCN(c2nc(OCCN3CCC(COc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N] | (2S,4R)-1-[(2S)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |
| 173 | 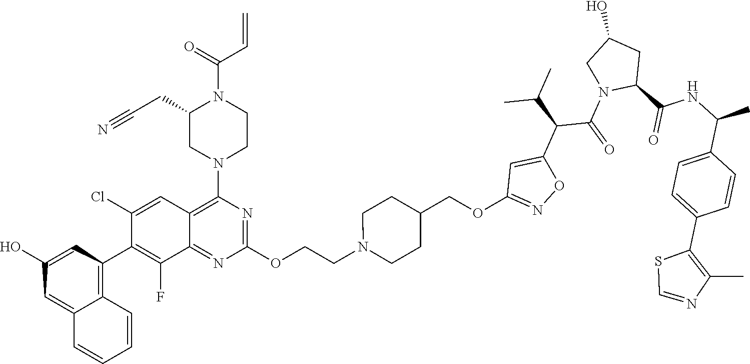 | (2S,4R)-1-[(2R)-2-[3-({1-[2- ({6-chloro-4-[(3S)-3- (cyanomethyl)-4-(prop-2- enoyl)piperazin-1-yl]-8-fluoro- 7-(3-hydroxynaphthalen-1- yl)quinazolin-2- yl}oxy)ethyl]piperidin-4- yl}methoxy)-1,2-oxazol-5-yl]- 3-methylbutanoyl]-4-hydroxy- N-[(1S)-1-[4-(4-methyl-1,3- thiazol-5- yl)phenyl]ethyl]pyrrolidine-2- carboxamide |

TABLE 8A
Degradation and characterization of exemplary bifunctional compounds of the present disclosure

| | | | KRas^{G12C}-HIBit | KRas^{G12C}-HiBit | KRas^{G12C}-HiBiT | KRas^{G12C}-HiBiT |
|---|---|---|---|---|---|---|
| | MH+ (1) | Mol | (SW1573; 0.1% | (SW1573; 0.1% | (SW1573; 0.5% | (SW1573; 0.5% |
| Ex | [MH+ (2)] | Weight | DMSO) DC₅₀* | DMSO) Dₘₐₓ** | DMSO) IC₅₀*** | DMSO) Dₘₐₓ** |
| 1 | 1187.50 | 1187.75 | C | | | |
| | [1189.50] | | | | | |
| 2 | 1169.60 | 1169.75 | C | | | |
| | [1171.60] | | | | | |
| 3 | 1169.50 | 1169.75 | A | A | | |
| | [1171.60] | | | | | |
| 4 | 1169.50 | 1169.75 | B | B | | |
| | [1171.60] | | | | | |
| 5 | 1169.50 | 1169.75 | C | | | |
| | [1171.50] | | | | | |
| 6 | 1143.60 | 1143.76 | C | | | |
| | [1145.60] | | | | | |
| 7 | 1173.60 | 1173.79 | C | | | |
| | [1175.60] | | | | | |
| 8 | 1104.60 | 1104.72 | C | | | |
| | [1106.60] | | | | | |
| 9 | 1104.60 | 1104.72 | B | A | | |
| | [1106.60] | | | | | |
| 10 | 1104.60 | 1104.72 | C | | | |
| | [1106.60] | | | | | |
| 11 | 1132.60 | 1132.78 | B | B | | |
| | [1134.60] | | | | | |
| 12 | 1144.64 | 1144.79 | B | B | B | B |
| | [1146.64] | | | | | |
| 13 | 1144.64 | 1144.79 | B | B | B | B |
| | [1146.64] | | | | | |
| 14 | 1132.58 | 1132.71 | C | | C | |
| | [1134.58] | | | | | |
| 15 | 1161.62 | 1161.75 | B | A | B | A |
| | [1163.62] | | | | | |
| 16 | 1187.40 | 1187.75 | B | A | B | A |
| | [1189.40] | | | | | |
| 17 | 1143.40 | 1143.76 | B | B | B | A |
| | [1145.40] | | | | | |
| 18 | 1132.40 | 1132.71 | C | | | |
| | [1134.40] | | | | | |
| 19 | 1114.40 | 1114.72 | C | | | |
| | [1116.40] | | | | | |
| 20 | 1114.40 | 1114.72 | B | B | B | B |
| | [1116.40] | | | | | |
| 21 | 1169.40 | 1169.75 | B | A | B | A |
| | 1169.50 | | | | | |
| | [1171.40 | | | | | |
| | 1171.60] | | | | | |
| 22 | 1169.40 | 1169.75 | B | C | | |
| | [1171.40] | | | | | |
| 23 | 1171.40 | 1171.81 | C | | | |
| | [1173.50] | | | | | |
| 24 | 1128.30 | 1128.36 | C | C | | |
| 25 | 1104.20 | 1104.72 | B | B | | |
| | [1106.20] | | | | | |
| 26 | 1173.20 | 1174.18 | C | B | | |
| | [1175.10] | | | | | |
| 27 | 1136.40 | 1136.74 | C | C | | |
| 28 | 1158.50 | 1158.82 | B | C | | |
| | [1160.50] | | | | | |
| 29 | 1110.50 | 1110.37 | C | B | C | B |
| 30 | 1124.50 | 1124.40 | C | C | C | |
| 31 | 1140.40 | 1140.40 | C | B | C | B |
| 32 | 1173.30 | 1173.79 | B | B | B | A |
| | [1175.30] | | | | | |
| 33 | 1187.40 | 1187.81 | B | B | B | B |
| | [1189.40] | | | | | |
| 34 | 1074.30 | 1074.70 | B | C | | |
| | [1076.30] | | | | | |
| 35 | 1198.50 | 1198.88 | C | | | |
| | [1200.50] | | | | | |
| 36 | 1132.30 | 1132.78 | B | B | B | B |
| | [1134.30] | | | | | |
| 37 | 1102.40 | 1102.75 | B | B | | |
| | [1104.40] | | | | | |
| 38 | 1088.40 | 1088.73 | C | | | |
| | [1090.40] | | | | | |
| 39 | 1162.30 | 1162.80 | B | B | B | B |
| | [1164.30] | | | | | |
| 40 | 1144.50 | 1144.79 | C | B | C | B |
| | [1146.50] | | | | | |
| 41 | 1076.20 | 1076.67 | C | B | | |
| | [1078.20] | | | | | |
| 42 | 1090.40 | 1090.70 | C | C | | |
| | [1092.40] | | | | | |
| 43 | 1104.40 | 1104.72 | B | B | B | A |
| | [1106.40] | | | | | |
| 44 | 1130.40 | 1130.74 | C | | | |
| | [1132.40] | | | | | |
| 45 | 1162.50 | 1162.80 | B | C | | |
| | [1164.50] | | | | | |
| 46 | 1159.50 | 1159.80 | B | B | B | B |
| | [1161.50] | | | | | |
| 47 | 1201.50 | 1201.88 | C | | | |
| | [1203.50] | | | | | |
| 48 | 1201.30 | 1201.88 | B | C | | |
| | [1203.30] | | | | | |
| 49 | 1215.40 | 1215.91 | B | C | | |
| | [1217.30] | | | | | |
| 50 | 1171.30 | 1171.86 | C | B | | |
| | [1173.30] | | | | | |
| 51 | 1119.20 | 1119.74 | C | C | | |
| | [1121.20] | | | | | |
| 52 | | 1214.92 | C | | | |
| 53 | 1203.30 | 1203.86 | B | C | | |
| | [1205.30] | | | | | |
| 54 | 1216.30 | 1216.90 | A | | | |
| | [1218.30] | | | | | |
| 55 | 1216.40 | 1216.90 | B | C | | |
| | [1218.40] | | | | | |
| 56 | 1214.40 | 1214.92 | C | | | |
| | [1216.40] | | | | | |
| 57 | | 1202.87 | B | C | | |
| 58 | 1124.40 | 1124.40 | C | | C | |
| 59 | | 1158.82 | C | B | | |
| 60 | 1178.30 | 1178.80 | B | C | B | B |
| | [1180.30] | | | | | |
| 61 | 1116.20 | 1116.71 | B | C | C | B |
| | [1118.30] | | | | | |
| 62 | 1144.30 | 1144.79 | B | B | | |
| | [1146.30] | | | | | |
| 63 | 1104.30 | 1104.72 | B | B | B | A |
| | 1104.60 | | | | | |
| | [1106.60] | | | | | |
| 64 | 1118.20 | 1118.75 | B | B | B | B |
| | [1120.30] | | | | | |
| 65 | | 1118.75 | B | B | B | A |
| 66 | 1134.30 | 1134.75 | B | B | B | B |
| | [1136.30] | | | | | |
| 67 | 1148.30 | 1148.78 | B | B | B | B |
| | [1150.30] | | | | | |
| 68 | 1132.30 | 1132.78 | B | B | B | B |
| | [1134.30] | | | | | |
| 69 | 1232.30 | 1232.85 | B | B | | |
| | [1234.30] | | | | | |
| 70 | 1232.30 | 1232.85 | C | | | |
| | [1234.30] | | | | | |
| 71 | 1188.20 | 1188.80 | B | C | | |
| | [1190.30] | | | | | |
| 72 | 1188.20 | 1188.80 | C | | | |
| | [1190.30] | | | | | |
| 73 | 1492.50 | 1492.82 | C | | | |
| 74 | 1404.50 | 1404.71 | C | | | |
| 75 | 1276.30 | 1276.90 | C | C | | |
| | [1278.30] | | | | | |
| 76 | 1276.30 | 1276.90 | C | | | |
| | [1278.30] | | | | | |
| 77 | 1144.20 | 1144.75 | B | C | | |
| | [1146.20] | | | | | |
| 78 | 1144.20 | 1144.75 | C | | | |
| | [1146.20] | | | | | |
| 79 | 1320.30 | 1320.96 | C | | | |
| | [1322.30] | | | | | |
| 80 | 1320.30 | 1320.96 | C | B | | |
| | [1322.30] | | | | | |
| 81 | 1316.50 | 1316.61 | C | | | |
| 82 | 1148.46 | 1148.78 | C | B | C | C |
| | [1150.46] | | | | | |
| 83 | 1544.69 | 1545.25 | C | | | |
| | [1546.69] | | | | | |
| 84 | 1456.64 | 1457.15 | C | | | |
| | [1458.64] | | | | | |
| 85 | 1148.47 | 1148.78 | C | | | |
| | 1148.46 | | | | | |
| | [1150.47 | | | | | |
| | 1150.46] | | | | | |
| 86 | 1148.47 | 1148.78 | B | B | B | B |
| | 1148.46 | | | | | |
| | 1148.40 | | | | | |
| | [1150.47 | | | | | |
| | 1150.46 | | | | | |
| 87 | 1092.49 | 1092.31 | C | B | | |
| 88 | 1368.59 | 1369.04 | C | | | |
| | [1370.59] | | | | | |
| 89 | 1092.50 | 1092.31 | B | B | C | |
| 95 | | 1143.76 | | | B | A |
| 96 | | 1169.80 | | | A | A |
| 97 | 1141.41 | 1141.79 | | | C | A |
| | [1143.41] | | | | | |
| 98 | 1189.39 | 1189.76 | | | B | A |
| | [1191.39] | | | | | |
| 99 | 1157.42 | 1157.79 | | | C | A |
| | [1159.42] | | | | | |
| 100 | 1157.27 | 1157.79 | | | B | C |
| | [1159.27] | | | | | |
| 101 | 1133.43 | 1133.70 | | | C | |
| | [1135.43] | | | | | |
| 102 | 1121.42 | 1121.76 | | | B | A |
| | [1123.42] | | | | | |
| 103 | 1151.41 | 1151.69 | | | B | |
| | [1153.41] | | | | | |
| 104 | 1139.37 | 1139.73 | | | B | C |
| | [1141.37] | | | | | |
| 105 | 1139.37 | 1139.73 | | | C | |
| | [1141.37] | | | | | |
| 106 | 1139.37 | 1139.73 | | | C | |
| | [1141.37] | | | | | |
| 107 | 1150.39 | 1150.75 | | | A | B |
| | [1152.39] | | | | | |
| 108 | 1150.38 | 1150.75 | | | C | |
| | [1152.38] | | | | | |
| 109 | | 1168.75 | | | A | B |
| 110 | | 1143.76 | | | B | C |
| 111 | | 1143.76 | | | B | B |
| 112 | | 1151.69 | | | B | A |
| 113 | | 1168.75 | | | C | |
| 114 | | 1154.79 | | | B | B |
| 115 | | 1175.73 | | | B | A |
| 116 | | 1133.70 | | | A | A |
| 117 | | 1158.66 | | | A | B |
| 118 | | 1158.66 | | | B | |
| 119 | | 1118.75 | | | B | A |
| 120 | | 1174.79 | | | B | B |
| 121 | | 1175.71 | | | A | B |
| 122 | | 1175.71 | | | C | |
| 123 | | 1171.75 | | | A | A |
| 124 | | 1154.70 | | | A | B |
| 125 | | 1154.70 | | | B | |
| 126 | | 1171.75 | | | C | |
| 127 | | 1183.82 | | | B | B |
| 128 | | 1131.75 | | | B | A |
| 129 | | 1144.70 | | | B | A |
| 130 | | 1183.82 | | | B | A |
| 131 | | 1153.76 | | | A | A |
| 132 | | 1153.76 | | | C | |
| 133 | | 1157.72 | | | A | A |
| 134 | | 1157.72 | | | C | |
| 135 | | 1141.79 | | | B | B |
| 136 | | 1126.71 | | | B | A |
| 137 | | 1150.75 | | | B | C |
| 138 | | 1122.68 | | | A | A |
| 139 | | 1150.75 | | | A | A |
| 140 | | 1129.73 | | | B | B |
| 141 | | 1140.67 | | | A | A |
| 142 | | 1129.73 | | | B | B |
| 143 | | 1152.77 | | | B | A |
| 144 | | 1152.77 | | | B | C |
| 145 | | 1154.79 | | | B | A |
| 146 | | 1100.69 | | | C | |
| 147 | | 1100.69 | | | B | A |
| 148 | | 1156.80 | | | B | B |
| 149 | | 1157.79 | | | B | A |
| 150 | | 1161.75 | | | B | A |
| 151 | | 1161.75 | | | A | A |
| 152 | | 1157.79 | | | B | A |
| 153 | | 1153.76 | | | B | A |
| 154 | | 1139.73 | | | C | |
| 155 | | 1171.75 | | | B | C |
| 156 | | 1140.67 | | | A | A |
| 157 | | 1122.68 | | | A | A |
| 158 | | 1140.67 | | | C | |
| 159 | | 1122.68 | | | C | |
| 160 | | 1067.60 | | | C | |
| 161 | | 1067.60 | | | A | B |
| 162 | 1143.25 | 1143.76 | | | C | C |
| | [1145.25] | | | | | |
| 163 | 1143.40 | 1143.76 | | | B | A |
| 164 | | 1242.89 | | | B | B |
| 165 | | 1200.85 | | | B | B |
| 166 | | 1157.72 | | | B | B |
| 167 | | 1157.72 | | | A | A |
| 168 | | 1157.72 | | | B | A |
| 169 | | 1139.73 | | | A | A |
| 170 | | 1191.78 | | | B | A |
| 171 | | 1157.72 | | | C | |
| 172 | | 1139.73 | | | C | |
| 173 | | 1139.73 | | | A | A |

*DC₅₀ (nM) C ≥ 1000; 100 ≤ B < 1000; A < 100
**D_{Max}(%): C ≤ 35; 35 < B < 70; A ≥ 70
***IC50 (nM) C ≥ 1000; 100 ≤ B < 1000; A < 100

TABLE 8B
Degradation and characterization of exemplary bifunctional
compounds of the present disclosure

| | KRas^{G12C}-HiBiT | KRas^{G12C}-HiBiT | KRas^{G12C}-HiBiT | KRas^{G12C}-HiBiT |
|---|---|---|---|---|
| | (MiaPaCa; 0.1% | (MiaPaCa; 0.1% | (MiaPaCa; 0.5% | (MiaPaCa; 0.5% |
| Ex | DMSO) IC₅₀*** | DMSO) D_{Max}** | DMSO) IC₅₀*** | DMSO) D_{Max}** |
| 1 | C | | | |
| 2 | C | | | |
| 3 | B | A | A | A |
| 4 | B | C | B | B |
| 5 | B | C | | |
| 6 | C | B | | |
| 7 | C | B | | |
| 8 | C | | | |
| 9 | B | A | B | A |
| 10 | C | | C | |
| 11 | B | B | | |
| 12 | B | C | B | C |
| 13 | B | B | B | B |
| 14 | C | | C | |
| 15 | B | B | B | A |
| 16 | B | B | B | A |
| 17 | B | A | B | A |
| 18 | C | | | |
| 19 | C | | | |
| 20 | B | B | B | B |
| 21 | B | A | B | A |
| 22 | B | C | | |
| 23 | C | | | |
| 24 | C | C | | |
| 25 | B | C | | |
| 26 | C | | | |
| 27 | C | | | |
| 28 | C | | | |
| 29 | C | B | C | B |
| 30 | C | | C | |
| 31 | C | C | C | C |
| 32 | B | B | B | A |
| 33 | B | B | B | B |
| 34 | C | | | |
| 35 | C | | | |
| 36 | B | C | B | B |
| 37 | B | C | | |
| 38 | C | | | |
| 39 | B | C | C | |
| 40 | B | C | C | C |
| 41 | B | C | | |
| 42 | C | | | |
| 43 | B | B | B | B |
| 44 | C | | | |
| 45 | C | | | |
| 46 | B | C | B | B |
| 47 | C | | | |
| 48 | C | | | |
| 49 | C | | | |
| 50 | C | B | | |
| 51 | B | C | | |
| 52 | C | | | |
| 53 | C | | | |
| 54 | C | | | |
| 55 | C | | | |
| 56 | C | | | |
| 57 | C | | | |
| 58 | C | | C | |
| 59 | C | C | | |
| 60 | C | | B | C |
| 61 | C | | C | |
| 62 | B | C | | |
| 63 | B | B | B | B |
| 64 | B | C | B | B |
| 65 | B | B | B | B |
| 66 | B | C | B | B |
| 67 | B | B | B | B |
| 68 | B | C | B | B |
| 69 | C | | | |
| 70 | | | | |
| 71 | B | | | |
| 72 | | | | |
| 73 | | | | |
| 74 | | | | |
| 75 | C | | | |
| 76 | | | | |
| 77 | B | C | | |
| 78 | | | | |
| 79 | | | | |
| 80 | C | | | |
| 81 | | | | |
| 82 | C | B | C | B |
| 83 | | | | |
| 84 | | | | |
| 85 | C | | | |
| 86 | B | B | B | B |
| 87 | C | | | |
| 88 | | | | |
| 89 | C | | C | |
| 95 | | | C | A |
| 96 | | | B | A |
| 97 | | | C | |
| 98 | | | B | A |
| 99 | | | C | A |
| 100 | | | C | C |
| 101 | | | C | |
| 102 | | | B | A |
| 103 | | | C | |
| 104 | | | B | C |
| 105 | | | A | C |
| 106 | | | A | C |
| 107 | | | A | B |
| 108 | | | B | C |
| 109 | | | A | A |
| 110 | | | B | C |
| 111 | | | B | B |
| 112 | | | B | A |
| 113 | | | C | |
| 114 | | | B | C |
| 115 | | | B | A |
| 116 | | | B | A |
| 117 | | | A | A |
| 118 | | | C | |
| 119 | | | B | A |
| 120 | | | B | B |
| 121 | | | B | A |
| 122 | | | C | |
| 123 | | | A | A |
| 124 | | | A | B |
| 125 | | | C | |
| 126 | | | C | |
| 127 | | | B | B |
| 128 | | | B | B |
| 129 | | | B | A |
| 130 | | | B | B |
| 131 | | | B | A |
| 132 | | | C | |
| 133 | | | B | A |
| 134 | | | C | |
| 135 | | | B | B |
| 136 | | | B | A |
| 137 | | | C | |
| 138 | | | B | A |
| 139 | | | B | A |
| 140 | | | C | B |
| 141 | | | B | A |
| 142 | | | C | B |
| 143 | | | B | A |
| 144 | | | C | |
| 145 | | | B | B |
| 146 | | | C | B |
| 147 | | | B | B |
| 148 | | | B | B |
| 149 | | | B | A |
| 150 | | | C | A |
| 151 | | | B | A |
| 152 | | | B | B |
| 153 | | | B | A |
| 154 | | | B | C |
| 155 | | | B | C |
| 156 | | | B | A |
| 157 | | | B | A |
| 158 | | | C | |
| 159 | | | C | |
| 160 | | | B | C |
| 161 | | | B | B |
| 162 | | | C | B |
| 163 | | | B | A |
| 164 | | | C | B |
| 165 | | | C | B |
| 166 | | | C | |
| 167 | | | A | A |
| 168 | | | B | A |
| 169 | | | B | A |
| 170 | | | B | B |
| 171 | | | C | |
| 172 | | | C | |
| 173 | | | A | A |

*DC₅₀ (nM) C ≥ 1000; 100 ≤ B < 1000; A < 100
**D_{Max} (%): C ≤ 35; 35 < B < 70; A ≥ 70
***IC50 (nM) C ≥ 1000; 100 ≤ B < 1000; A < 100

TABLE 9
Exemplary Compounds of the Present Disclosure

| Ex. | 1H NMR | Synthetic Scheme |
|---|---|---|
| 95 | 1H-NMR (400 MHz, CD3OD) δ 8.86 (s, 1H), 8.05 (s, | Prepared in an analogous manner to |
| | 1H), 7.74 (d, J = 8.8 Hz, 1H), 7.55-7.35 (m, 5H), 7.28- | (2S,4R)-1-[(2S)-2-[[2-[4-[(2S)-2-[[6- |
| | 7.17 (m, 3H), 7.05-7.00 (m, 1H), 6.94-6.75 (m, 1H), | chloro-4-[(3S)-3-(cyanomethyl)-4- |
| | 6.36-6.26 (m, 1H), 5.90-5.79 (m, 1H), 5.18-5.04 (m, | prop-2-enoyl-piperazin-1-yl]-8- |
| | 1H), 5.00-4.95 (m, 1H), 4.70-4.62 (m, 4H), 4.52-4.40 | fluoro-7-(3-hydroxy-1- |
| | (m, 3H), 3.93-3.67 (m, 4H), 3.40 (d, J = 6.0 Hz, 2H), | naphthyl)quinazolin-2- |
| | 3.21-3.14 (m, 2H), 3.09-2.82 (m, 4H), 2.46 (s, 3H), | yl]oxymethyl]pyrrolidin-1- |
| | 2.34-2.15 (m, 3H), 2.01-1.90 (m, 1H), 1.87-1.64 (m, | yl]butoxy]acetyl]amino]-3,3- |
| | 3H), 1.56-1.27 (m, 6H), 1.25-0.97 (m, 14H). | dimethyl-butanoyl]-4-hydroxy-N- |
| | | [(1S)-1-[4-(4-methylthiazol-5- |
| | | yl)phenyl]ethyl]pyrrolidine-2- |
| | | carboxamide |
| 96 | 1H-NMR (400 MHz, CD3OD) δ 8.86 (s, 1H), 8.05 (s, | Specific Scheme Provided |
| | 1H), 7.74 (d, J = 8.8 Hz, 1H), 7.55-7.35 (m, 5H), 7.28- | |
| | 7.17 (m, 3H), 7.05-7.00 (m, 1H), 6.94-6.75 (m, 1H), | |
| | 6.36-6.26 (m, 1H), 5.90-5.79 (m, 1H), 5.18-5.04 (m, | |
| | 1H), 5.00-4.95 (m, 1H), 4.70-4.62 (m, 4H), 4.52-4.40 | |
| | (m, 3H), 3.93-3.67 (m, 4H), 3.40 (d, J = 6.0 Hz, 2H), | |
| | 3.21-3.14 (m, 2H), 3.09-2.82 (m, 4H), 2.46 (s, 3H), | |
| | 2.34-2.15 (m, 3H), 2.01-1.90 (m, 1H), 1.87-1.64 (m, | |
| | 3H), 1.56-1.27 (m, 6H), 1.25-0.97 (m, 14H). | |
| 97 | 1H-NMR (400 MHz, CD3OD) δ 8.86 (s, 1H), 8.45 (s, | Prepared in an analogous manner to |
| | 1H), 8.00 (d, J = 7.2 Hz, 1H), 7.85 (d, J = 8.4 Hz, 1H), | (2S,4R)-1-[(2S)-2-[2-({1-[2-({6- |
| | 7.54 (t, J = 7.6 Hz, 1H), 7.44-7.36 (m, 5H), 7.31-7.22 | chloro-4-[(3S)-3-(cyanomethyl)-4- |
| | (m, 2H), 6.94-6.71 (m, 1H), 6.31 (d, J = 16.4 Hz, 1H), | (prop-2-enoyl)piperazin-1-yl]-8- |
| | 5.84 (d, J = 10.0 Hz, 1H), 5.14-5.02 (m, 1H), 5.02-4.96 | fluoro-7-(8-methylnaphthalen-1- |
| | (m, 1H), 4.84-4.79 (m, 2H), 4.64-4.59 (m, 1H), 4.56- | yl)quinazolin-2- |
| | 4.43 (m, 3H), 4.40-4.32 (m, 1H), 4.21-4.08 (m, 1H), | yl}oxy)ethyl]piperidin-4- |
| | 4.08-3.77 (m, 5H), 3.77-3.59 (m, 4H), 3.52-3.42 (m, | yl}methoxy)acetamido]-3,3- |
| | 4H), 3.11-2.90 (m, 4H), 2.46 (s, 3H), 2.40 (dt, J = 9.2, | dimethylbutanoyl]-4-hydroxy-N- |
| | 4.4 Hz, 1H), 2.07 (d, J = 2.8 Hz, 3H), 2.03-1.84 (m, | [(1S)-1-[4-(4-methyl-1,3-thiazol-5- |
| | 4H), 1.61 (d, J = 8.4 Hz, 2H), 1.46 (d, J = 7.2 Hz, 3H), | yl)phenyl]ethyl]pyrrolidine-2- |
| | 1.07-0.96 (m, 9H). | carboxamide |
| 98 | (m, 4H), 7.07-6.69 (m, 1H), 6.51 (s, 1H), 6.37-6.22 (m, | Prepared in an analogous manner to |
| | 1H), 5.95-5.69 (m, 1H), 5.19-5.00 (m, 2H), 4.61-4.39 | (2S,4R)-1-[(2S)-2-[[2-[[1-[2-[7-[6- |
| | (m, 6H), 4.23-3.78 (m, 6H), 3.14-2.97 (m, 4H), 2.85 (s, | amino-4-methyl-3-(trifluoromethyl)- |
| | 4H), 2.62-2.41 (m, 9H), 2.24-2.18 (m, 1H), 2.00-1.79 | 2-pyridyl]-6-chloro-4-[(3S)-3- |
| | (m, 5H), 1.58-1.40 (m, 6H), 1.14-1.01 (m, 12H). | (cyanomethyl)-4-prop-2-enoyl- |
| | | piperazin-1-yl]-8-fluoro-quinazolin-2- |
| | | yl]oxyethyl]-4- |
| | | piperidyl]methoxy]acetyl]amino]-3,3- |
| | | dimethyl-butanoyl]-4-hydroxy-N- |
| | | [(1S)-1-[4-(4-methylthiazol-5- |
| | | yl)phenyl]ethyl]pyrrolidine-2- |
| | | carboxamide |
| 99 | 1H-NMR (400 MHz, CD3OD) δ 8.92-8.81 (m, 1H), | Prepared in an analogous manner to |
| | 8.03 (s, 1H), 7.69 (br d, J = 8.4 Hz, 1H), 7.49 (t, J = 7.2 | (2S,4R)-1-[(2S)-2-[[2-[[1-[2-[7-(3- |
| | Hz, 1H), 7.44-7.35 (m, 4H), 7.27 (br t, J = 8.0 Hz, | amino-1-isoquinolyl)-6-chloro-4- |
| | 1H), 7.12-7.05 (m, 1H), 6.98-6.77 (m, 1H), 6.76- | [(3S)-3-(cyanomethyl)-4-prop-2- |
| | 6.72 (m, 1H), 6.30 (br d, J = 16.8 Hz, 1H), 5.83 (br d, J = | enoyl-piperazin-1-yl]-8-fluoro- |
| | 9.6 Hz, 1H), 5.10 (br s, 1H), 5.01-4.95 (m, 1H), 4.68 | quinazolin-2-yl]oxyethyl]-4- |
| | (s, 1H), 4.63 (br t, J = 5.6 Hz, 2H), 4.61-4.50 (m, 2H), | piperidyl]methoxy]acetyl]amino]-3,3- |
| | 4.49-4.38 (m, 3H), 4.23-4.03 (m, 1H), 4.03-3.91 (m, | dimethyl-butanoyl]-4-hydroxy-N- |
| | 2H), 3.83 (br d, J = 11.2 Hz, 2H), 3.77-3.64 (m, 2H), | [(1S)-1-[4-(4-methylthiazol-5- |
| | 3.40 (br d, J = 6.4 Hz, 2H), 3.16-2.99 (m, 4H), 2.98- | yl)phenyl]ethyl]pyrrolidine-2- |
| | 2.93 (m, 3H), 2.86 (br t, J = 5.6 Hz, 2H), 2.46 (s, 3H), | carboxamide |
| | 2.20 (br t, J = 10.8 Hz, 3H), 1.95 (ddd, J = 4.4, 9.2, 13.2 | |
| | Hz, 1H), 1.83-1.66 (m, 3H), 1.58-1.43 (m, 3H), 1.42- | |
| | 1.30 (m, 2H), 1.07-0.98 (m, 9H). | |
| 100 | ¹H-NMR (400 MHz, CDC1₃) δ 8.75-8.65 (m, 1H), 7.73 | Specific Scheme Provided |
| | (dd, J = 8.4, 4.0 Hz, 1H), 7.62 (s, 1H), 7.44-7.29 (m, | |
| | 7H), 7.25-7.09 (m, 3H), 6.71-6.51 (m, 1H), 6.41 (d, J = | |
| | 16.4 Hz, 1H), 5.84 (d, J = 10.0 Hz, 1H), 5.34-4.98 (m, | |
| | 2H), 4.81-4.66 (m, 1H), 4.61-4.44 (m, 2H), 4.43-4.05 | |
| | (m, 4H), 3.85-3.29 (m, 14H), 3.07-2.76 (m, 2H), 2.57- | |
| | 2.40 (m, 4H), 2.19-1.91 (m, 5H), 1.81-1.52 (m, 8H), | |
| | 1.50-1.39 (m, 3H), 1.03 (s, 9H). | |
| 101 | ¹H-NMR (400 MHz, CD₃OD) δ 8.05 (s, 1H), 7.75 (d, | Prepared in an analogous manner to |
| | J = 4.0 Hz, 1H), 7.47 (s, 2H), 7.46-7.38 (m, 4H), 7.36- | (2S,4R)-1-[(2R)-2-(3-{7-[2-({6- |
| | 7.26 (m, 2H), 7.25-7.22 (m, 1H), 7.04 (d, J = 4.0 Hz, | chloro-4-[(3S)-3-(cyanomethyl)-4- |
| | 1H), 6.96-6.76 (m, 1H), 6.35-6.32 (m, 1H), 6.31-6.28 | (prop-2-enoyl)piperazin-1-yl]-8- |
| | (m, 1H), 5.88-5.86 (m, 1H), 5.85-5.83 (m, 1H), 5.01(d, | fluoro-7-(3-hydroxynaphthalen-1- |
| | J = 4.0 Hz, 1H), 4.86-4.57 (m, 3H), 4.49-4.45 (m, 3H), | yl)quinazolin-2-yl}oxy)ethyl]-2,7- |
| | 3.86-3.74 (m, 4H), 3.71-3.62 (m, 8H), 3.01 (s, 2H), 2.84 | diazaspiro[3.5]nonan-2-yl}-1,2- |
| | (s, 2H), 2.56 (s, 4H), 2.37-2.35 (m, 1H), 2.21-2.19 (m, | oxazol-5-yl)-3-methylbutanoyl]-4- |
| | 1H), 1.96-1.94 (m, 1H), 1.85-1.80 (m, 3H), 1.59 (d, J = | hydroxy-N-[(1S)-1-[4-(1-methyl-1H- |
| | 3.2 Hz, 1H), 1.47 (d, J = 4.0 Hz, 3H), 1.33-1.29 (m, | pyrazol-5- |
| | 3H), 1.05 (d, J = 4.0 Hz, 3H), 0.90 (d, J = 4.0 Hz, 3H). | yl)phenyl]ethyl]pyrrolidine-2- |
| | | carboxamide |
| 102 | ¹H-NMR (400 MHz, CD₃OD) δ 8.93-8.84 (m, 1H), 8.53 | Specific Scheme Provided |
| | (s, 1H), 8.02 (s, 1H), 7.49-7.35 (m, 4H), 6.99-6.67 (m, | |
| | 2H), 6.45 (d, J = 2.4 Hz, 1H), 6.32 (d, J = 16.8 Hz, 1H), | |
| | 5.86 (d, J = 10.0 Hz, 1H), 5.01 (q, J = 6.8 Hz, 1H), | |
| | 4.78-4.74 (m, 2H), 4.71 (s, 1H), 4.60-4.36 (m, 5H), | |
| | 4.20-3.93 (m, 3H), 3.89-3.72 (m, 4H), 3.65-3.45 (m, | |
| | 5H), 3.10-2.95 (m, 2H), 2.80-2.58 (m, 2H), 2.54-2.46 | |
| | (m, 3H), 2.44-2.16 (m, 5H), 2.09-1.80 (m, 8H), 1.60- | |
| | 1.46 (m, 5H), 1.09-0.99 (m, 9H). | |
| 103 | ¹H-NMR (400 MHz, CD₃OD) δ 8.08 (s, 1H), 7.76 (d, | Prepared in an analogous manner to |
| | J = 4.0 Hz, 1H), 7.48-7.47 (m, 1H), 7.42-7.36 (m, 5H), | (2S,4R)-1-[(2R)-2-(3-{7-[2-({6- |
| | 7.27 (s, 1H), 7.26-7.04 (m, 2H), 7.03 (s, 1H), 6.35-6.32 | chloro-4-[(3S)-3-(cyanomethyl)-4-(2- |
| | (m, 1H), 5.89 (s, 1H), 5.42-5.31 (m, 2H), 5.00-4.89 (m, | fluoroprop-2-enoyl)piperazin-1-yl]-8- |
| | 1H), 4.88-4.87 (m, 1H), 4.75 (s, 2H), 4.59-4.52 (m, 5H), | fluoro-7-(3-hydroxynaphthalen-1- |
| | 3.86 (s, 1H), 3.83-3.74 (m, 3H), 3.72-3.66 (m, 9H), | yl)quinazolin-2-yl}oxy)ethyl]-2,7- |
| | 3.31-3.25 (m, 2H), 3.25-3.01 (m, 6H), 1.98-1.96 (m, | diazaspiro[3.5]nonan-2-yl}-1,2- |
| | 1H), 1.95-1.93 (m, 1H), 1.92-1.91 (m, 5H), 1.48 (d, J = | oxazol-5-yl)-3-methylbutanoyl]-4- |
| | 7.6 Hz, 3H), 1.05 (d, J = 7.6 Hz, 3H), 0.94 (d, J = 7.6 | hydroxy-N-[(1S)-1-[4-(1-methyl-1H- |
| | Hz, 3H). | pyrazol-5- |
| | | yl)phenyl]ethyl]pyrrolidine-2- |
| | | carboxamide |
| 104 | ¹H-NMR (400 MHz, CD₃OD) δ 8.87-8.86 (m, 1H), 8.08 | Prepared in an analogous manner to |
| | (s, 1H), 7.76-7.74 (m, 1H), 7.45-7.40 (m, 6H), 7.27- | (2S,4R)-1-[(2R)-2-[3-({1-[2-({6- |
| | 7.26 (m, 1H), 7.20-7.18 (m, 2H), 7.04-7.03 (m, 1H), | chloro-4-[(3S)-3-(cyanomethyl)-4- |
| | 6.33-6.29 (m, 1H), 5.98 (s, 1H), 5.93-5.86 (m, 1H), | (prop-2-enoyl)piperazin-1-yl]-8- |
| | 5.04-4.86 (m, 2H), 4.75-4.53 (m, 2H), 4.52-4.49 (m, | fluoro-7-(3-hydroxynaphthalen-1- |
| | 5H), 4.07-4.06 (m, 2H), 3.84-3.81 (m, 2H), 3.68 (d, J = | yl)quinazolin-2- |
| | 3.6 Hz 1H), 3.66-3.62 (m, 1H), 3.48-3.46 (m, 3H), 3.25- | yl}oxy)ethyl]piperidin-4- |
| | 3.23 (m, 2H), 3.01 (s, 2H), 2.54-2.52 (m, 2H), 2.48-2.36 | yl}methoxy)-1,2-oxazol-5-yl]-3- |
| | (m, 3H), 2.34-2.32 (m, 1H), 2.15-2.13 (m, 1H), 1.98- | methylbutanoyl]-4-hydroxy-N-[(1S)- |
| | 1.92 (m, 5H), 1.59-1.57 (m, 2H), 1.51 (d, J = 3.6 Hz | 1-[4-(4-methyl-1,3-thiazol-5- |
| | 3H), 1.05 (d, J = 3.6 Hz, 3H), 0.90 (d, J = 3.6 Hz, 3H). | yl)phenyl]ethyl]pyrrolidine-2- |
| | | carboxamide |
| 105 | ¹H-NMR (400 MHz, CD₃OD) δ 8.85 (s, 1H), 8.45 (s, | Prepared in an analogous manner |
| | 1H), 8.09 (s, 1H), 7.75 (d, J = 8.4 Hz, 1H), 7.48-7.37 | to(2S,4R)-1-[(2R)-2-[3-({1-[2-({6- |
| | (m, 3H), 7.35-7.28 (m, 2H), 7.27 (d, J = 2.4 Hz, 1H), | chloro-4-[(3S)-3-(cyanomethyl)-4- |
| | 7.25-7.14 (m, 2H), 7.03 (d, J = 2.4 Hz, 1H), 6.91-6.72 | (prop-2-enoyl)piperazin-1-yl]-8- |
| | (m, 1H), 6.31 (d, J = 16.8 Hz, 1H), 6.00 (s, 1H), 5.84 (d, | fluoro-7-(3-hydroxynaphthalen-1- |
| | J = 10.5 Hz, 1H), 5.14-4.95 (m, 2H), 4.77 (s, 2H), 4.56- | yl)quinazolin-2- |
| | 4.36 (m, 4H), 4.26-3.97 (m, 3H), 3.94-3.69 (m, 4H), | yl}oxy)ethyl]piperidin-4- |
| | 3.67-3.34 (m, 6H), 3.13-2.92 (m, 2H), 2.80 (s, 2H), | yl}methoxy)-1,2-oxazol-5-yl]-3- |
| | 2.51-2.30 (m, 5H), 2.04-1.82 (m, 4H), 1.63-1.38 (m, | methylbutanoyl]-4-hydroxy-N-[(1S)- |
| | 5H), 1.08-0.74 (m, 6H). | 1-[4-(4-methyl-1,3-thiazol-5- |
| | | yl)phenyl]ethyl]pyrrolidine-2- |
| | | carboxamide |
| 106 | ¹H-NMR (400 MHz, CD₃OD) δ 8.89-8.85 (m, 1H), 8.47 | Prepared in an analogous manner |
| | (s, 1H), 8.09 (s, 1H), 7.75 (d, J = 8.0 Hz, 1H), 7.47-7.32 | to(2S,4R)-1-[(2R)-2-[3-({1-[2-({6- |
| | (m, 5H), 7.29-7.14 (m, 3H), 7.03 (d, J = 2.4 Hz, 1H), | chloro-4-[(3S)-3-(cyanomethyl)-4- |
| | 6.95-6.74 (m, 1H), 6.31 (d, J = 17.2 Hz, 1H), 6.00-5.88 | (prop-2-enoyl)piperazin-1-yl]-8- |
| | (m, 1H), 5.85 (d, J = 10.8 Hz, 1H), 5.15-4.91 (m, 2H), | fluoro-7-(3-hydroxynaphthalen-1- |
| | 4.79-4.73 (m, 2H), 4.55-4.46 (m, 2H), 4.43 (dd, J = 9.6, | yl)quinazolin-2- |
| | 4.0 Hz, 1H), 4.39-4.10 (m, 2H), 4.10-3.99 (m, 2H), | yl}oxy)ethyl]piperidin-4- |
| | 3.97-3.55 (m, 6H), 3.54-3.41 (m, 2H), 3.30-3.21 (m, | yl}methoxy)-1,2-oxazol-5-yl]-3- |
| | 2H), 3.08-2.98 (m, 2H), 2.81-2.63 (m, 2H), 2.51-2.45 | methylbutanoyl]-4-hydroxy-N-[(1S)- |
| | (m, 3H), 2.42-2.25 (m, 2H), 2.13-1.83 (m, 4H), 1.64- | 1-[4-(4-methyl-1,3-thiazol-5- |
| | 1.45 (m, 5H), 1.05-0.98 (m, 3H), 0.91-0.85 (m, 3H). | yl)phenyl]ethyl]pyrrolidine-2- |
| | | carboxamide |
| 107 | ¹H-NMR (400 MHz, CD₃OD) δ 8.89-8.84 (m, 1H), 8.06 | Specific Scheme Provided |
| | (s, 1H), 7.75 (d, J = 8.0 Hz, 1H), 7.50-7.34 (m, 5H), | |
| | 7.29-7.15 (m, 3H), 7.04 (d, J = 2.4 Hz, 1H), 6.96-6.75 | |
| | (m, 1H), 6.31 (d, J = 17.6 Hz, 1H), 5.89-5.78 (m, 2H), | |
| | 5.05-4.99 (m, 1H), 4.64 (t, J = 5.2 Hz, 2H), 4.49-4.37 | |
| | (m, 3H), 4.28-4.06 (m, 1H), 3.90-3.79 (m, 2H), 3.76- | |
| | 3.43 (m, 9H), 3.08-2.97 (m, 2H), 2.91-2.81 (m, 2H), | |
| | 2.74-2.51 (m, 4H), 2.50-2.45 (m, 3H), 2.39-2.30 (m, | |
| | 1H), 2.23-2.12 (m, 1H), 2.10-1.73 (m, 6H), 1.60-1.49 | |
| | (m, 3H), 1.04 (d, J = 6.4 Hz, 3H), 0.92-0.84 (m, 3H). | |
| 108 | ¹H-NMR (400 MHz, CD₃OD) δ 8.90-8.84 (m, 1H), 8.06 | Prepared in an analogous manner to |
| | (s, 1H), 7.74 (d, J = 8.0 Hz, 1H), 7.42-7.40 (m, 2H), | (2S,4R)-1-[(2R)-2-(3-{7-[2-({6- |
| | 7.38-7.32 (m, 2H), 7.28-7.22 (m, 2H), 7.21-7.16 (m, | chloro-4-[(3S)-3-(cyanomethyl)-4- |
| | 1H), 7.04 (d, J = 4.0 Hz, 1H), 6.90-6.75 (m, 1H), 6.31 | (prop-2-enoyl)piperazin-1-yl]-8- |
| | (d, J = 16.0 Hz, 1H), 5.90-5.80 (m, 2H), 5.18-5.04 (m, | fluoro-7-(3-hydroxynaphthalen-1- |
| | 1H), 5.02-4.95 (m, 1H), 4.66-4.61 (m, 2H), 4.60-4.53 | yl)quinazolin-2-yl}oxy)ethyl]-2,7- |
| | (m, 3H), 4.51-4.40 (m, 3H), 4.24-4.08 (m, 1H), 3.91- | diazaspiro[3.5]nonan-2-yl}-1,2- |
| | 3.82 (m, 1H), 3.78-3.55 (m, 9H), 3.03 (s, 2H), 2.84 (t, | oxazol-5-yl)-3-methylbutanoyl]-4- |
| | J = 4.0 Hz, 2H), 2.57 (s, 3H), 2.49-2.45 (m, 3H), 2.40- | hydroxy-N-[(1S)-1-[4-(4-methyl-1,3- |
| | 2.34 (m, 1H), 2.27-2.17 (m, 1H), 2.00-1.92 (m, 1H), | thiazol-5-yl)phenyl]ethyl]pyrrolidine- |
| | 1.87-1.73 (m, 4H), 1.48 (d, J = 4.0 Hz, 3H), 1.05 (d, J = | 2-carboxamide |
| | 4.0 Hz, 3H), 0.91 (d, J = 4.0 Hz, 3H). | |
| 109 | ¹H-NMR (400 MHz, CD₃OD) δ 8.90-8.60 (m, 1H), 8.43 | Specific Scheme Provided |
| | (s, 1H), 8.08 (s, 1H), 7.76 (d, J = 8.4 Hz, 1H), 7.49-7.33 | |
| | (m, 5H), 7.30-7.14 (m, 3H), 7.03 (d, J = 2.4 Hz, 1H), | |
| | 5.88-5.81 (m, 1H), 5.47-5.27 (m, 2H), 5.06-5.00 (m, | |
| | 1H), 4.74 (t, J = 4.8 Hz, 2H), 4.57-4.41 (m, 4H), 3.88- | |
| | 3.79 (m, 2H), 3.75-3.67 (m, 5H), 3.66-3.43 (m, 3H), | |
| | 3.25-3.17 (m, 2H), 3.16-2.88 (m, 6H), 2.50-2.45 (m, | |
| | 3H), 2.41-2.31 (m, 1H), 2.22-2.12 (m, 1H), 2.08-1.91 | |
| | (m, 5H), 1.61-1.49 (m, 3H), 1.41-1.18 (m, 2H), 1.04 (d, | |
| | J = 6.4 Hz, 3H), 0.92-0.84 (m, 3H). | |
| 110 | ¹H-NMR (400 MHz, CD₃OD) δ 8.92-8.80 (m, 1H), | Prepared in an analogous manner to |
| | 8.04 (s, 1H), 7.74 (d, J = 8.4 Hz, 1H), 7.44-7.35 (m, | (2S,4R)-1-[(2S)-2-[2-({1-[2-({6- |
| | 5H), 7.25 (d, J = 2.4 Hz, 1H), 7.23-7.15 (m, 2H), 7.03 | chloro-4-[(3S)-3-(cyanomethyl)-4- |
| | (d, J = 2.4 Hz, 1H), 6.98-6.72 (m, 1H), 6.30 (d, J = | (prop-2-enoyl)piperazin-1-yl]-8- |
| | 16.4 Hz, 1H), 5.83 (d, J = 10.0 Hz, 1H), 5.19-4.91 (m, | fluoro-7-(3-hydroxynaphthalen-1- |
| | 2H), 4.80-4.51 (m, 5H), 4.50-4.39 (m, 3H), 4.02- | yl)quinazolin-2- |
| | 3.90 (m, 2H), 3.87-3.63 (m, 4H), 3.40 (d, J = 6.4 Hz, | yl}oxy)ethyl]piperidin-4- |
| | 2H), 3.12 (d, J = 11.2 Hz, 2H), 3.03 (d, J = 5.6 Hz, 2H), | yl}methoxy)acetamido]-3,3- |
| | 2.87 (t, J = 5.6 Hz, 2H), 2.46 (s, 3H), 2.27-2.14 (m, | dimethylbutanoyl]-4-hydroxy-N- |
| | 3H), 2.00-1.90 (m, 1H), 1.85-1.66 (m, 3H), 1.58- | [(1S)-1-[4-(4-methyl-1,3-thiazol-5- |
| | 1.44 (m, 3H), 1.44-1.26 (m, 3H), 1.06-0.98 (m, 9H) | yl)phenyl]ethyl]pyrrolidine-2- |
| | | carboxamide |
| 111 | ¹H-NMR (400 MHz, CD₃OD) δ 8.87 (s, 1H), 8.49 (br s, | Specific Scheme Provided |
| | 1H), 8.10 (s, 1H), 7.75-7.59 (m, 1H), 7.57-7.23 (m, | |
| | 6H), 7.19-7.07 (m, 1H), 7.02-6.90 (m, 1H), 6.88- | |
| | 6.73 (m, 1H), 6.42-6.19 (m, 1H), 5.97-5.76 (m, 1H), | |
| | 5.18-4.95 (m, 3H), 4.72-4.38 (m, 7H), 4.27-4.09 (m, | |
| | 1H), 4.06-3.68 (m, 6H), 3.64-3.39 (m, 6H), 3.13- | |
| | 2.96 (m, 2H), 2.94-2.66 (m, 2H), 2.47 (s, 3H), 2.29- | |
| | 2.14 (m, 1H), 2.10-1.80 (m, 4H), 1.70-1.37 (m, 5H), | |
| | 1.03 (s, 9H). | |
| 112 | ¹H-NMR (400 MHz, CD₃OD) δ 8.10 (s, 1H), 7.49-7.48 | Specific Scheme Provided |
| | (m, 1H), 7.45 (s, 1H), 7.43-7.40 (m, 5H), 7.28 (s, 1H), | |
| | 7.27-7.22 (m, 2H), 7.03 (s, 1H), 6.34 (d, J = 2.0 Hz, 1H), | |
| | 5.88 (s, 1H), 5.37-5.32 (m, 2H), 5.05-4.85 (m, 2H), | |
| | 4.60-4.44 (m, 6H), 3.85-3.82 (m, 5H), 3.74 (m, 6H), | |
| | 3.65-3.63 (m, 2H), 3.62-3.61 (m 3H), 3.31-3.10 (m, | |
| | 1H), 3.08 (d, J = 3.6 Hz, 3H), 2.37-2.34 (m, 1H), 2.07- | |
| | 2.04 (m, 1H), 2.02-1.99 (m, 4H), 1.97-1.95 (m, 1H), | |
| | 1.59-1.51 (m, 3H), 1.32-1.3 (m, 2H), 1.05 (d, J = 7.6 | |
| | Hz, 3H), 0.91-0.87 (m, 3H). | |
| 113 | ¹H-NMR (400 MHz, CD₃OD) δ 8.91-8.81 (m, 1H), | Prepared in an analogous manner to |
| | 8.46-8.36 (m, 1H), 8.07 (s, 1H), 7.74 (d, J = 8.0 Hz, | (2S,4R)-1-[(2R)-2-(3-{7-[2-({6- |
| | 1H), 7.49-7.32 (m, 5H), 7.28-7.14 (m, 3H), 7.03 (d, J = | chloro-4-[(3S)-3-(cyanomethyl)-4-(2- |
| | 4.0 Hz, 1H), 5.89 (s, 1H), 5.45-5.27 (m, 2H), 5.04-4.88 | fluoroprop-2-enoyl)piperazin-1-yl]-8- |
| | (m, 3H), 4.79-4.69 (m, 2H), 4.61-4.36 (m, 4H), 4.28- | fluoro-7-(3-hydroxynaphthalen-1- |
| | 4.11 (m, 1H), 3.88-3.77 (m, 1H), 3.76-3.56 (m, 8H), | yl)quinazolin-2-yl}oxy)ethyl]-2,7- |
| | 3.27-3.19 (m, 2H), 3.15-2.89 (m, 5H), 2.50-2.42 (m, | diazaspiro[3.5]nonan-2-yl}-1,2- |
| | 3H), 2.40-2.30 (m, 1H), 2.28-2.17 (m, 1H), 2.01-1.91 | oxazol-5-yl)-3-methylbutanoyl]-4- |
| | (m, 5H), 1.48 (d, J = 8.0 Hz, 3H), 1.05 (d, J = 8.0 Hz, | hydroxy-N-[(1S)-1-[4-(4-methyl-1,3- |
| | 3H), 0.92-0.80 (m, 3H). | thiazol-5-yl)phenyl]ethyl]pyrrolidine- |
| | | 2-carboxamide |
| 114 | ¹H-NMR (400 MHz, CD₃OD) δ 8.91-8.78 (m, 1H), 8.42 | Specific Scheme Provided |
| | (s, 1H), 7.88 (s, 1H), 7.74 (d, J = 8.4 Hz, 1H), 7.48-7.31 | |
| | (m, 5H), 7.29-7.10 (m, 3H), 7.04-6.96 (m, 1H), 6.94- | |
| | 6.70 (m, 1H), 6.30 (d, J = 16.8 Hz, 1H), 5.84 (d, J = | |
| | 11.2 Hz, 1H), 5.03-4.96 (m, 3H), 4.63-4.51 (m, 2H), | |
| | 4.47-4.28 (m, 2H), 4.24-3.69 (m, 10H), 3.68-3.38 (m, | |
| | 4H), 3.08-2.60 (m, 8H), 2.50-2.39 (m, 3H), 2.36-2.06 | |
| | (m, 5H), 2.03-1.88 (m, 1H), 1.59-1.43 (m, 3H), 1.10- | |
| | 0.97 (m, 9H). | |
| 115 | ¹H-NMR (400 MHz, CD₃OD) δ 8.90-8.83 (m, 1H), 8.53 | Specific Scheme Provided |
| | (s, 1H), 7.97 (s, 1H), 7.51-7.33 (m, 4H), 6.99-6.72 (m, | |
| | 1H), 6.60 (s, 1H), 6.30 (d, J = 16.4 Hz, 1H), 5.83 (d, J = | |
| | 10.4 Hz, 1H), 5.14-5.05 (m, 1H), 5.02-4.96 (m, 1H), | |
| | 4.82-4.66 (m, 4H), 4.61-4.52 (m, 1H), 4.50-4.34 (m, | |
| | 3H), 4.20-4.08 (m, 1H), 4.06-3.91 (m, 2H), 3.89-3.80 | |
| | (m, 2H), 3.77-3.64 (m, 2H), 3.53-3.41 (m, 4H), 3.30- | |
| | 3.18 (m, 2H), 3.10-2.96 (m, 2H), 2.83-2.60 (m, 2H), | |
| | 2.50-2.46 (m, 3H), 2.44 (s, 3H), 2.30-2.15 (m, 1H), | |
| | 2.00-1.81 (m, 4H), 1.60-1.45 (m, 5H), 1.08-0.98 (m, | |
| | 9H). | |
| 116 | ¹H-NMR (400 MHz, DMSO-d₆) δ 8.41 (d, J = 7.6 Hz, | Specific Scheme Provided |
| | 1H), 8.15 (s, 1H), 8.11 (s, 1H), 7.81 (d, J = 7.6 Hz, 1H), | |
| | 7.50-7.46 (m, 4H), 7.45-7.44 (m, 2H), 7.39 (s, 1H), | |
| | 7.37-7.23 (m, 2H), 7.07 (s, 1H), 6.37 (s, 1H), 6.21 (d, | |
| | J = 15.6 Hz, 1H), 5.83-5.81 (m, 1H), 5.10 (s, 1H), 4.99- | |
| | 4.91 (m, 1H), 4.48-4.45 (m, 1H), 4.37 (t, J = 7.6 Hz | |
| | 2H), 4.34-4.26 (m, 3H), 4.25-4.22 (m, 2H), 3.86-3.85 | |
| | (m, 1H), 3.84 (s, 3H), 3.58-3.53 (m, 5H), 2.69-2.66 (m, | |
| | 3H), 2.42-2.41 (m, 4H), 2.33 (s, 1H), 2.31 (m, 2H), | |
| | 2.02-1.99 (m, 1H), 1.77-1.71 (m, 6H), 1.44 (d, J = 7.2 | |
| | Hz, 1H), 1.38 (d, J = 7.2 Hz, 3H), 0.96-0.93 (m, 4H), | |
| | 0.82-0.77 (m, 4H). | |
| 117 | ¹H-NMR (400 MHz, DMSO-d₆) δ 10.06 (s, 1H), 8.43 | Specific Scheme Provided |
| | (d, J = 7.6 Hz, 1H), 8.11 (s, 1H), 7.81 (d, J = 8.4 Hz, 1H), | |
| | 7.48-7.42 (m, 4H), 7.40-7.36 (m, 2H), 7.29 (d, J = 2.0 | |
| | Hz, 1H), 7.23-7.19 (m, 2H), 7.06 (d, J = 2.4 Hz, 1H), | |
| | 6.37 (d, J = 2.0 Hz, 1H), 6.14 (s, 1H), 5.42 (dd, J = 4.0, | |
| | 18.0 Hz, 1H), 5.32 (d, J = 19.2 Hz, 1H), 5.11 (d, J = 3.2 | |
| | Hz, 1H), 5.00-4.82 (m, 2H), 4.54-4.45 (m, 2H), 4.40- | |
| | 4.33 (m, 2H), 4.33-4.15 (m, 5H), 3.84 (s, 3H), 3.73- | |
| | 3.53 (m, 6H), 3.06 (dd, J = 6.0, 17.2 Hz, 2H), 2.95-2.78 | |
| | (m, 4H), 2.46-2.31 (m, 2H), 2.30-2.15 (m, 1H), 2.07- | |
| | 1.73 (m, 6H), 1.38 (d, J = 6.8 Hz, 3H), 0.98-0.93 (m, | |
| | 3H), 0.83-0.77 (m, 3H). | |
| 118 | ¹H-NMR (400 MHz, DMSO-d₆) δ 10.04 (s, 1H), 8.35 | Prepared in an analogous manner to |
| | (d, J = 7.6 Hz, 1H), 8.12 (s, 1H), 7.81 (d, J = 8.4 Hz, 1H), | (2S,4R)-1-[(2R)-2-[3-({1-[2-({6- |
| | 7.47-7.41 (m, 5H), 7.35-7.28 (m, 3H), 7.22 (d, J = 4.0 | chloro-4-[(3S)-3-(cyanomethyl)-4-(2- |
| | Hz, 2H), 7.07 (d, J = 2.4 Hz, 1H), 6.35 (d, J = 1.6 Hz, 1H), | fluoroprop-2-enoyl)piperazin-1-yl]-8- |
| | 6.13 (s, 1H), 5.42 (dd, J = 4.0, 18.0 Hz, 1H), 5.26 (d, | fluoro-7-(3-hydroxynaphthalen-1- |
| | J = 11.2 Hz, 1H), 5.11 (d, J = 3.6 Hz, 1H), 4.95-4.84 (m, | yl)quinazolin-2-yl}oxy)ethyl]-4- |
| | 2H), 4.55-4.48 (s, 2H), 4.42 (t, J = 7.6 Hz, 1H), 4.37- | fluoropiperidin-4-yl}methoxy)-1,2- |
| | 4.34 (m, 1H), 4.31-4.19 (m, 5H), 3.83 (s, 3H), 3.58- | oxazol-5-yl]-3-methylbutanoyl]-4- |
| | 3.44 (m, 5H), 3.06 (dd, J = 6.0, 17.2 Hz, 2H), 2.82 (s, | hydroxy-N-[(1S)-1-[4-(1-methyl-1H- |
| | 4H), 2.32-2.20 (m, 2H), 2.08-2.01 (m, 1H), 1.97- | pyrazol-5- |
| | 1.69 (m, 6H), 1.35 (d, J = 6.8 Hz, 3H), 0.96 (d, J = 6.4 Hz, | yl)phenyl]ethyl]pyrrolidine-2- |
| | 3H), 0.82 (d, J = 6.8 Hz, 3H). | carboxamide |
| 119 | ¹H-NMR (400 MHz, DMSO-d₆) δ 10.01 (d, J = 4.0 Hz, | Specific Scheme Provided |
| | 1H), 8.99-8.95 (m, 1H), 8.43 (d, J = 8.0 Hz, 1H), 7.91 | |
| | (s, 1H), 7.80 (d, J = 8.0 Hz, 1H), 7.45-7.40 (m, 3H), | |
| | 7.35-7.32 (m, 2H), 7.29-7.25 (m, 2H), 7.20 (d, J = 4.0 | |
| | Hz, 2H), 7.05 (d, J = 4.0, 1H), 6.90-6.77 (m, 1H), 6.23- | |
| | 6.14 (m, 1H), 5.77-5.72 (m, 1H), 5.12 (d, J = 4.0 Hz, | |
| | 1H), 4.86 (s, 1H), 4.52 (d, J = 8.0 Hz, 1H), 4.48-4.34 | |
| | (m, 4H), 4.30-4.08 (m, 4H), 3.90 (s, 2H), 3.72-3.61 | |
| | (m, 2H), 3.59-3.50 (m, 3H), 3.02-2.88 (m, 2H), 2.72- | |
| | 2.65 (m, 2H), 2.44 (s, 4H), 2.12-1.92 (m, 3H), 1.67- | |
| | 1.48 (m, 3H), 1.40-1.28 (m, 7H), 1.26-1.07 (m, 3H), | |
| | 0.91 (s, 9H) . | |
| 120 | ¹H-NMR (400 MHz, CD₃OD) δ 8.97-8.70 (m, 1H), 8.39 | Specific Scheme Provided |
| | (s, 1H), 8.08 (s, 1H), 7.74 (d, J = 8.0 Hz, 1H), 7.45-7.31 | |
| | (m, 5H), 7.30-7.12 (m, 3H), 7.07-6.98 (m, 1H), 6.93- | |
| | 6.72 (m, 1H), 6.31 (d, J = 16.8 Hz, 1H), 5.84 (d, J = | |
| | 10.4 Hz, 1H), 5.13-4.90 (m, 2H), 4.75-4.36 (m, 7H), | |
| | 4.12-3.43 (m, 6H), 3.28-2.62 (m, 12H), 2.46-2.34 (m, | |
| | 6H), 2.26-1.78 (m, 6H), 1.45 (d, J = 7.2 Hz, 3H), 1.06- | |
| | 0.97 (m, 9H). | |
| 121 | ¹H-NMR (400 MHz, CD₃OD) δ 8.90-8.84 (m, 1H), 8.64 | Specific Scheme Provided |
| | (d, J = 7.2 Hz, 1H), 8.08 (s, 1H), 7.75 (d, J = 8.4 Hz, | |
| | 1H), 7.46-7.38 (m, 5H), 7.28-7.17 (m, 3H), 7.03 (d, J = | |
| | 2.6 Hz, 1H), 6.11-5.95 (m, 1H), 5.51-5.25 (m, 2H), | |
| | 5.10-4.94 (m, 2H), 4.72-4.36 (m, 6H), 4.26 (d, J = 19.2 | |
| | Hz, 3H), 3.89-3.45 (m, 7H), 3.25-2.84 (m, 6H), 2.52- | |
| | 2.45 (m, 3H), 2.39-1.84 (m, 8H), 1.58-1.46 (m, 3H), | |
| | 1.05 (d, J = 6.4 Hz, 3H), 0.91-0.82 (m, 3H). | |
| 122 | ¹H-NMR (400 MHz, CD₃OD) δ 8.85 (s, 1H), 8.55 (d, | Prepared in an analogous manner to |
| | J = 7.6 Hz, 1H), 8.09 (s, 1H), 7.74 (d, J = 8.4 Hz, 1H), | (2S,4R)-1-[(2R)-2-[3-({1-[2-({6- |
| | 7.42-7.32 (m, 5H), 7.29-7.17 (m, 3H), 7.02 (d, J = 2.4 | chloro-4-[(3S)-3-(cyanomethyl)-4-(2- |
| | Hz, 1H), 6.06 (s, 1H), 5.46-5.30 (m, 2H), 5.03-4.93 (m, | fluoroprop-2-enoyl)piperazin-1-yl]-8- |
| | 2H), 4.73-4.40 (m, 6H), 4.38-4.25 (m, 3H), 3.90-3.52 | fluoro-7-(3-hydroxynaphthalen-1- |
| | (m, 11H), 3.08 (d, J = 6.4 Hz, 2H), 2.45 (s, 3H), 2.37- | yl)quinazolin-2-yl}oxy)ethyl]-4- |
| | 1.87 (m, 8H), 1.48 (d, J = 7.2 Hz, 3H), 1.05 (d, J = 6.4 | fluoropiperidin-4-yl}methoxy)-1,2- |
| | Hz, 3H), 0.91 (d, J = 6.4 Hz, 3H). | oxazol-5-yl]-3-methylbutanoyl]-4- |
| | | hydroxy-N-[(1S)-1-[4-(4-methyl-1,3- |
| | | thiazol-5-yl)phenyl]ethyl]pyrrolidine- |
| | | 2-carboxamide |
| 123 | ¹H-NMR (400 MHz, CD₃OD) δ 8.89-8.86 (m, 1H), | Specific Scheme Provided |
| | 8.49-8.45 (m, 1H), 8.09 (s, 1H), 7.77-7.72 (m, 1H), | |
| | 7.47-7.43 (m, 2H), 7.42-7.36 (m, 3H), 7.27 (d, J = 2.0 | |
| | Hz, 1H), 7.23-7.16 (m, 2H), 7.03 (d, J = 2.0 Hz, 1H), | |
| | 6.04-5.93 (m, 1H), 5.45-5.29 (m, 2H), 5.07-4.93 (m, | |
| | 2H), 4.84-4.71 (m, 3H), 4.59-4.48 (m, 3H), 4.45-4.38 | |
| | (m, 1H), 4.05-3.98 (m, 2H), 3.83 (dd, J = 10.8, 4.0 Hz, | |
| | 2H), 3.77-3.57 (m, 4H), 3.54-3.36 (m, 3H), 3.29-3.24 | |
| | (m, 1H), 3.16-3.04 (m, 4H), 2.49-2.45 (m, 3H), 2.40- | |
| | 2.32 (m, 1H), 2.22-2.14 (m, 1H), 2.06-1.95 (m, 1H), | |
| | 1.94-1.86 (m, 2H), 1.64 (d, J = 13.6 Hz, 2H), 1.58 (d, | |
| | J = 7.2 Hz, 3H), 1.15-1.12 (m, 3H), 1.07-1.03 (m, 3H), | |
| | 0.91-0.86 (m, 3H). | |
| 124 | ¹H-NMR (400 MHz, CD₃OD) δ 8.52 (s, 1H), 8.07 (s, | Specific Scheme Provided |
| | 1H), 7.75 (d, J = 8.4 Hz, 1H), 7.50-7.37 (m, 6H), 7.29- | |
| | 7.14 (m, 3H), 7.05-7.00 (m, 1H), 6.36-6.30 (m, 1H), | |
| | 6.02-5.93 (m, 1H), 5.46-5.25 (m, 2H), 5.08-4.95 (m, | |
| | 2H), 4.78-4.70 (m, 2H), 4.57-4.37 (m, 4H), 4.05-3.95 | |
| | (m, 2H), 3.86-3.45 (m, 9H), 3.26-3.00 (m, 6H), 2.97- | |
| | 2.78 (m, 2H), 2.43-2.14 (m, 2H), 2.06-1.90 (m, 1H), | |
| | 1.88-1.73 (m, 2H), 1.64-1.48 (m, 5H), 1.33-1.25 (m, | |
| | 1H), 1.14-1.08 (m, 3H), 1.05 (d, J = 6.8 Hz, 3H), 0.92- | |
| | 0.83 (m, 3H). | |
| 125 | ¹H-NMR (400 MHz, CD₃OD) δ 8.49 (s, 1H), 8.08 (s, | Prepared in an analogous manner to |
| | 1H), 7.74 (d, J = 8.0 Hz, 1H), 7.51-7.33 (m, 6H), 7.29- | (2S,4R)-1-[(2R)-2-[3-({1-[2-({6- |
| | 7.13 (m, 3H), 7.05-6.99 (m, 1H), 6.37-6.28 (m, 1H), | chloro-4-[(3S)-3-(cyanomethyl)-4-(2- |
| | 6.07-5.97 (m, 1H), 5.48-5.26 (m, 2H), 5.03-4.95 (m, | fluoroprop-2-enoyl)piperazin-1-yl]-8- |
| | 2H), 4.79-4.74 (m, 2H), 4.60-4.12 (m, 5H), 4.05-3.93 | fluoro-7-(3-hydroxynaphthalen-1- |
| | (m, 2H), 3.90-3.54 (m, 9H), 3.37-3.33 (m, 1H), 3.30- | yl)quinazolin-2-yl}oxy)ethyl]-4- |
| | 2.91 (m, 7H), 2.43-2.30 (m, 1H), 2.27-2.15 (m, 1H), | methylpiperidin-4-yl}methoxy)-1,2- |
| | 2.00-1.78 (m, 3H), 1.68-1.42 (m, 5H), 1.15-1.08 (m, | oxazol-5-yl]-3-methylbutanoyl]-4- |
| | 3H), 1.07-0.93 (m, 3H), 0.93-0.81 (m, 3H). | hydroxy-N-[(1S)-1-[4-(1-methyl-1H- |
| | | pyrazol-5- |
| | | yl)phenyl]ethyl]pyrrolidine-2- |
| | | carboxamide |
| 126 | ¹H-NMR (400 MHz, CD₃OD) δ 8.89-8.82 (m, 1H), | Prepared in an analogous manner to |
| | 8.07-8.01 (m, 1H), 7.77-7.71 (m, 1H), 7.43-7.38 (m, | (2S,4R)-1-[(2R)-2-[3-({1-[2-({6- |
| | 3H), 7.37-7.32 (m, 2H), 7.27-7.21 (m, 2H), 7.20-7.15 | chloro-4-[(3S)-3-(cyanomethyl)-4-(2- |
| | (m, 1H), 7.05-6.99 (m, 1H), 6.04-5.96 (m, 1H), 5.45- | fluoroprop-2-enoyl)piperazin-1-yl]-8- |
| | 5.26 (m, 2H), 5.13-4.94 (m, 2H), 4.65-4.61 (m, 2H), | fluoro-7-(3-hydroxynaphthalen-1- |
| | 4.59-4.35 (m, 5H), 3.97-3.91 (m, 2H), 3.81-3.73 (m, | yl)quinazolin-2-yl}oxy)ethyl]-4- |
| | 2H), 3.72-3.62 (m, 3H), 3.18-3.02 (m, 2H), 2.91-2.84 | methylpiperidin-4-yl} methoxy)-1,2- |
| | (m, 2H), 2.76-2.67 (m, 2H), 2.59-2.50 (m, 2H), 2.49- | oxazol-5-yl]-3-methylbutanoyl]-4- |
| | 2.45 (m, 3H), 2.42-2.33 (m, 1H), 2.25-2.17 (m, 1H), | hydroxy-N-[(1S)-1-[4-(4-methyl-1,3- |
| | 1.98-1.91 (m, 1H), 1.76-1.54 (m, 3H), 1.50-1.41 (m, | thiazol-5-yl)phenyl]ethyl]pyrrolidine- |
| | 5H), 1.07-1.01 (m, 6H), 0.90 (d, J = 6.8 Hz, 3H). | 2-carboxamide |
| 127 | ¹H-NMR (400 MHz, DMSO-d₆) δ 10.04 (d, J = 2.8 Hz, | Prepared in an analogous manner to |
| | 1H), 8.99-8.98 (m, 1H), 8.44 (d, J = 8.0 Hz, 1H), 8.10 | (2S,4R)-1-[(2S)-2-[[2-[[1-[(1S,2S)-2- |
| | (s, 1H), 7.81 (d, J = 8.4 Hz, 1H), 7.45-7.41 (m, 3H), | [6-chloro-8-fluoro-7-(3-hydroxy-1- |
| | 7.37-7.32 (m, 2H), 7.30-7.16 (m, 4H), 7.10-7.05 (m, | naphthyl)-4-(4-prop-2- |
| | 1H), 7.00-6.79 (m, 1H), 6.21 (dd, J = 16.8, 2.0 Hz, 1H), | enoylpiperazin-1-yl)quinazolin-2- |
| | 5.84-5.75 (m, 1H), 5.42-5.33 (m, 1H), 5.14 (d, J = 2.8 | yl]oxycyclopentyl]-4- |
| | Hz, 1H), 5.04-4.82 (m, 3H), 4.58-4.48 (m, 1H), 4.47- | piperidyl]methoxy]acetyl]amino]-3,3- |
| | 4.17 (m, 6H), 4.14-4.03 (m, 1H), 3.93-3.81 (m, 4H), | dimethyl-butanoyl]-4-hydroxy-N- |
| | 3.64-3.51 (m, 5H), 3.10-2.87 (m, 4H), 2.45 (s, 3H), | [(1S)-1-[4-(4-methylthiazol-5- |
| | 2.14-1.89 (m, 4H), 1.82-1.71 (m, 1H), 1.69-1.42 (m, | yl)phenyl]ethyl]pyrrolidine-2- |
| | 5H), 1.39-1.29 (m, 3H), 1.28-1.11 (m, 2H), 0.91 (d, J = | carboxamide |
| | 2.0 Hz, 9H). | |
| 128 | ¹H-NMR (400 MHz, DMSO-d₆) δ 13.18 (s, 1H), 8.98 | Specific Scheme Provided |
| | (s, 1H), 8.45 (d, J = 7.6 Hz, 1H), 8.12 (s, 1H), 7.61-7.49 | |
| | (m, 2H), 7.45-7.32 (m, 5H), 7.28 (d, J = 9.6 Hz, 1H), | |
| | 6.95-6.77 (m, 1H), 6.21 (d, J = 18.0 Hz, 1H), 5.79 (d, | |
| | J = 8.8 Hz, 1H), 5.28-4.80 (m, 3H), 4.53 (d, J = 9.6 Hz, | |
| | 1H), 4.50-4.41 (m, 3H), 4.40-4.15 (m, 4H), 4.07 (s, | |
| | 1H), 3.90 (s, 2H), 3.58-3.55 (m, 1H), 3.41-3.30 (m, | |
| | 5H), 3.05-2.89 (m, 4H), 2.71 (t, J = 5.6 Hz, 2H), 2.45 (s, | |
| | 3H), 2.16 (d, J = 3.8 Hz, 3H), 2.10-1.98 (m, 3H), 1.78- | |
| | 1.71 (m, 1H), 1.69-1.52 (m, 3H), 1.34 (dd, J = 2.0, 6.8 | |
| | Hz, 3H), 1.26-1.13 (m, 2H), 0.92 (s, 9H). | |
| 129 | ¹H-NMR (400 MHz, DMSO-d₆) δ 10.04 (s, 1H), 8.47 | Specific Scheme Provided |
| | (d, J = 7.6 Hz, 1H), 8.14 (s, 1H), 8.12 (s, 1H), 7.82 (d, | |
| | J = 8.4 Hz, 1H), 7.50-7.42 (m, 5H), 7.37 (d, J = 8.4 Hz, | |
| | 2H), 7.32-7.27 (m, 3H), 7.22 (d, J = 4.0 Hz, 2H), 7.07 | |
| | (d, J = 2.4 Hz, 1H), 6.37 (d, J = 2.0 Hz, 1H), 5.48-5.36 | |
| | (m, 2H), 5.32-5.23 (m, 1H), 5.14 (d, J = 3.2 Hz, 1H), | |
| | 4.96-4.86 (m, 2H), 4.57-4.24 (m, 10H), 3.92 (s, 3H), | |
| | 3.84 (s, 4H), 3.62-3.53 (m, 2H), 3.11-2.97 (m, 3H), | |
| | 2.84-2.72 (m, 3H), 2.20-1.99 (m, 2H), 1.84-1.56 (m, | |
| | 4H), 1.36 (d, J = 7.2 Hz, 3H), 1.29-1.14 (m, 2H), 0.93 | |
| | (s, 9H). | |
| 130 | ¹H-NMR (400 MHz, DMSO-d₆) δ 10.04 (s, 1H), 8.99 | Prepared in an analogous manner to |
| | (s, 1H), 8.45 (d, J = 7.6 Hz, 1H), 8.11 (s, 1H), 7.82 (d, | (2S,4R)-1-[(2S)-2-[[2-[[1-[(1R,2R)-2- |
| | J = 8.4 Hz, 1H), 7.43 (d, J = 8.0 Hz, 4H), 7.38-7.32 (m, | [6-chloro-8-fluoro-7-(3-hydroxy-1- |
| | 2H), 7.32-7.16 (m, 5H), 7.08 (d, J = 2.0 Hz, 1H), 6.97- | naphthyl)-4-(4-prop-2- |
| | 6.79 (m, 1H), 6.22 (d, J = 17.2 Hz, 1H), 5.86-5.74 (m, | enoylpiperazin-1-yl)quinazolin-2- |
| | 1H), 5.40-5.35 (m, 1H), 5.14 (d, J = 3.6 Hz, 1H), 5.02- | yl]oxycyclopentyl]-4- |
| | 4.98 (m, 1H), 4.93-4.83 (m, 2H), 4.52 (d, J = 9.6 Hz, | piperidyl]methoxy]acetyl]amino]-3,3- |
| | 1H), 4.48-4.42 (m, 1H), 4.40-4.19 (m, 4H), 4.16-4.03 | dimethyl-butanoyl]-4-hydroxy-N- |
| | (m, 1H), 3.93-3.81 (m, 3H), 3.62-3.51 (m, 4H), 3.18- | [(1S)-1-[4-(4-methylthiazol-5- |
| | 2.86 (m, 7H), 2.46 (s, 3H), 2.13-1.89 (m, 4H), 1.81-1.63 | yl)phenyl]ethyl]pyrrolidine-2- |
| | (m, 2H), 1.63-1.43 (m, 4H), 1.39-1.32 (m, 3H), 1.21- | carboxamide [00248] |
| | 1.11 (m, 3H), 0.96-0.87 (m, 9H). | |
| 131 | ¹H-NMR (400 MHz, CD₃OD) δ 8.91-8.83 (m, 1H), | Specific Scheme Provided |
| | 8.55-8.49 (m, 1H), 8.13-8.03 (m, 1H), 7.80-7.71 (m, | |
| | 1H), 7.47-7.35 (m, 5H), 7.27 (d, J = 2.0 Hz, 1H), 7.25- | |
| | 7.15 (m, 2H), 7.06-7.01 (m, 1H), 6.96-6.76 (m, 1H), | |
| | 6.36-6.26 (m, 1H), 6.03-5.92 (m, 1H), 5.90-5.81 (m, | |
| | 1H), 5.18-4.94 (m, 2H), 4.76-4.70 (m, 2H), 4.62-4.57 | |
| | (m, 1H), 4.54-4.38 (m, 4H), 4.22-3.92 (m, 3H), 3.90- | |
| | 3.79 (m, 2H), 3.77-3.43 (m, 4H), 3.23-3.13 (m, 2H), | |
| | 3.08-2.98 (m, 3H), 2.96-2.80 (m, 2H), 2.49-2.45 (m, | |
| | 3H), 2.40-2.31 (m, 1H), 2.22-2.13 (m, 1H), 2.04-1.90 | |
| | (m, 1H), 1.86-1.75 (m, 2H), 1.61-1.49 (m, 5H), 1.14- | |
| | 1.08 (m, 3H), 1.05 (d, J = 6.4 Hz, 3H), 0.92-0.84 (m, | |
| | 3H). | |
| 132 | ¹H-NMR (400 MHz, CD₃OD) δ 8.91-8.83 (m, 1H), | Prepared in an analogous manner to |
| | 8.54-8.48 (m, 1H), 8.11-8.01 (m, 1H), 7.74 (d, J = 8.4 | (2S,4R)-1-[(2R)-2-[3-({1-[2-({6- |
| | Hz, 1H), 7.47-7.32 (m, 5H), 7.26 (s, 1H), 7.24-7.14 (m, | chloro-4-[(3S)-3-(cyanomethyl)-4- |
| | 2H), 7.03 (dd, J = 4.8, 2.4 Hz, 1H), 6.94-6.73 (m, 1H), | (prop-2-enoyl)piperazin-1-yl]-8- |
| | 6.35-6.25 (m, 1H), 6.04-5.96 (m, 1H), 5.89-5.78 (m, | fluoro-7-(3-hydroxynaphthalen-1- |
| | 1H), 5.15-4.93 (m, 2H), 4.77-4.69 (m, 2H), 4.61-4.34 | yl)quinazolin-2-yl}oxy)ethyl]-4- |
| | (m, 5H), 4.21-3.91 (m, 3H), 3.90-3.51 (m, 6H), 3.25- | methylpiperidin-4-yl}methoxy)-1,2- |
| | 3.14 (m, 2H), 3.08-2.97 (m, 3H), 2.95-2.81 (m, 2H), | oxazol-5-yl]-3-methylbutanoyl]-4- |
| | 2.50-2.43 (m, 3H), 2.42-2.32 (m, 1H), 2.26-2.16 (m, | hydroxy-N-[(1S)-1-[4-(4-methyl-1,3- |
| | 1H), 1.98-1.89 (m, 1H), 1.86-1.74 (m, 2H), 1.60-1.44 | thiazol-5-yl)phenyl]ethyl]pyrrolidine- |
| | (m, 5H), 1.12-1.06 (m, 3H), 1.05 (d, J = 6.8 Hz, 3H), | 2-carboxamide |
| | 0.92-0.82 (m, 3H). | |
| 133 | ¹H-NMR (400 MHz, CD₃OD) δ 8.88 (s, 1H), 8.46 (s, | Specific Scheme Provided |
| | 1H), 8.07 (s, 1H), 7.76 (d, J = 8.0 Hz, 1H), 7.49-7.34 | |
| | (m, 5H), 7.29-7.17 (m, 3H), 7.04 (d, J = 2.4 Hz, 1H), | |
| | 6.93-6.73 (m, 1H), 6.31 (d, J = 16.4 Hz, 1H), 6.06-5.95 | |
| | (m, 1H), 5.89-5.81 (m, 1H), 5.16-4.95 (m, 2H), 4.74- | |
| | 4.56 (m, 4H), 4.56-4.38 (m, 4H), 4.29-4.17 (m, 2H), | |
| | 3.85-3.82 (m, 2H), 3.78-3.57 (m, 3H), 3.56-3.44 (m, | |
| | 1H), 3.03 (s, 6H), 2.76-2.56 (m, 2H), 2.48 (s, 3H), 2.39- | |
| | 1.88 (m, 6H), 1.52 (d, J = 7.2 Hz, 3H), 1.05 (d, J = 6.8 | |
| | Hz, 3H), 0.88 (d, J = 6.8 Hz, 3H). | |
| 134 | ¹H-NMR (400 MHz, CD₃OD) δ 8.92-8.80 (m, 1H), | Prepared in an analogous manner to |
| | 8.51-8.38 (m, 1H), 8.07 (s, 1H), 7.74 (d, J = 8.4 Hz, | (2S,4R)-1-[(2R)-2-[3-({1-[2-({6- |
| | 1H), 7.51-7.30 (m, 5H), 7.29-7.11 (m, 3H), 7.03 (t, J = | chloro-4-[(3S)-3-(cyanomethyl)-4- |
| | 2.4 Hz, 1H), 6.96-6.66 (m, 1H), 6.31 (d, J = 16.8 Hz, | (prop-2-enoyl)piperazin-1-yl]-8- |
| | 1H), 6.10-5.97 (m, 1H), 5.84 (d, J = 10.0 Hz, 1H), 5.14- | fluoro-7-(3-hydroxynaphthalen-1- |
| | 4.98 (m, 2H), 4.85-4.32 (m, 8H), 4.21 (d, J = 19.6 Hz, | yl)quinazolin-2-yl}oxy)ethyl]-4- |
| | 2H), 3.96-3.50 (m, 6H), 3.20-2.93 (m, 6H), 2.73 (s, 2H), | fluoropiperidin-4-yl}methoxy)-1,2- |
| | 2.52-2.44 (m, 3H), 2.41-1.90 (m, 6H), 1.62-1.43 (m, | oxazol-5-yl]-3-methylbutanoyl]-4- |
| | 3H), 1.09-0.93 (m, 3H), 0.93-0.82 (m, 3H). | hydroxy-N-[(1S)-1-[4-(4-methyl-1,3- |
| | | thiazol-5-yl)phenyl]ethyl]pyrrolidine- |
| | | 2-carboxamide |
| 135 | ¹H-NMR (400 MHz, CD₃OD) δ 8.87 (s, 1H), 8.53 (s, | Specific Scheme Provided |
| | 1H), 8.11-7.93 (m, 2H), 7.84 (d, J = 7.6 Hz, 1H), 7.60- | |
| | 7.49 (m, 1H), 7.48-7.32 (m, 5H), 7.32-7.21 (m, 2H), | |
| | 7.01-6.72 (m, 1H), 6.40-6.24 (m, 1H), 5.84 (d, J = 8.4 | |
| | Hz, 1H), 5.07-5.00 (m, 1H), 4.75-4.40 (m, 10H), 4.05- | |
| | 3.67 (m, 6H), 3.62-3.31 (m, 4H), 3.25-2.96 (m, 4H), | |
| | 2.73-2.51 (m, 2H), 2.46 (s, 3H), 2.27-2.16 (m, 1H), 2.07 | |
| | (s, 3H), 2.00-1.77 (m, 4H), 1.62-1.47 (m, 2 H), 1.46 (d, | |
| | J = 5.2 Hz, 3H), 1.02 (s, 9H). | |
| 136 | ¹H-NMR (400 MHz, DMSO-d₆) δ 8.46 (d, J = 7.6 Hz, | Specific Scheme Provided |
| | 1H), 8.24 (s, 2H), 8.11 (s, 1H), 7.81 (d, J = 8.4 Hz, 1H), | |
| | 7.50-7.42 (m, 5H), 7.40-7.35 (m, 2H), 7.31-7.26 (m, | |
| | 2H), 7.23 (d, J = 4.0 Hz, 2H), 7.08 (d, J = 2.4 Hz, 1H), | |
| | 6.98-6.78 (m, 1H), 6.37 (d, J = 2.0 Hz, 1H), 6.22 (d, J = | |
| | 16.4 Hz, 1H), 5.80 (d, J = 9.2 Hz, 1H), 4.99 (d, J = 2.4 | |
| | Hz, 1H), 4.95-4.85 (m, 2H), 4.71-4.63 (m, 1H), 4.54 (d, | |
| | J = 9.6 Hz, 1H), 4.50-4.42 (m, 2H), 4.41-4.31 (m, 2H), | |
| | 4.25 (d, J = 15.6 Hz, 3H), 4.16-4.03 (m, 2H), 3.91 (s, | |
| | 2H), 3.84 (s, 5H), 3.12-3.03 (m, 3H), 3.01-2.92 (m, 3H), | |
| | 2.71 (t, J = 5.6 Hz, 3H), 2.12-1.98 (m, 2H), 1.76-1.53 | |
| | (m, 3H), 1.36 (d, J = 7.2 Hz, 3H), 1.27-1.14 (m, 2H), | |
| | 0.93 (s, 9H). | |
| 137 | 1H-NMR (400 MHz, DMSO-d6) δ 8.97-8.96 (m, 1H), | Prepared in an analogous manner to |
| | 8.35 (s, 1H), 8.18 (d, J = 8.0 Hz, 1H), 8.11 (s, 1H), 7.80 | (2S,4R)-1-[(2R)-2-(3-{7-[2-({6- |
| | (d, J = 8.0 Hz, 1H), 7.49-7.45 (m, 1H), 7.44-7.38 (m, | chloro-4-[(3S)-3-(cyanomethyl)-4- |
| | 4H), 7.32-7.27 (m, 4H), 7.21 (s, 1H), 7.18-7.14 (m, | (prop-2-enoyl)piperazin-1-yl]-8- |
| | 1H), 7.06 (d, J = 4.0 Hz, 1H), 6.23-6.18 (m, 1H), 5.84 | fluoro-7-(3-hydroxynaphthalen-1- |
| | (s, 1H), 5.81-5.76 (m, 1H), 5.08-5.13 (m, 1H), 5.00- | yl)quinazolin-2-yl}oxy)ethyl]-2,7- |
| | 4.96 (m, 1H), 4.89-4.85 (m, 1H), 4.49-4.42 (m, 3H), | diazaspiro[3.5]nonan-2-yl}-l,2- |
| | 4.40-4.37 (m, 1H), 4.28-4.24 (m, 2H), 4.23-4.20 (m, | oxazol-5-yl)-3-methylbutanoyl]-4- |
| | 1H), 4.10-4.00 (m, 1H), 3.90-3.80 (m, 1H) 3.68 (d, J = | hydroxy-N-[(1S)-1-[4-(4-methyl-1,3- |
| | 8.0 Hz, 2H), 3.56 (s, 2H), 3.53 (d, J = 4.0 Hz, 3H), | thiazol-5-yl)phenyl]ethyl]pyrrolidine- |
| | 3.49 (s, 4H), 3.09-3.02 (m, 2H), 2.68-2.64 (m, 3H), | 2-carboxamide |
| | 2.43 (s, 3H), 2.33-2.32 (m, 1H), 1.73-1.68 (m, 1H), | |
| | 1.64 (s, 3H), 1.44 (d, J = 8.0 Hz, 1H), 1.34 (d, J = 4.0 | |
| | Hz, 3H), 1.23 (s, 1H), 0.94 (d, J = 8.0 Hz, 3H), 0.80 (s, | |
| | 2H), 0.73 (d, J = 4.0 Hz, 1H). | |
| 138 | 1H-NMR (400 MHz, CD3OD) δ 8.47 (s, 1H), 8.10 (s, | Specific Scheme Provided |
| | 1H), 7.75 (d, J = 8.4 Hz, 1H), 7.52-7.35 (m, 6H), 7.31- | |
| | 7.13 (m, 3H), 7.03 (d, J = 2.4 Hz, 1H), 6.98-6.74 (m, | |
| | 1H), 6.38-6.26 (m, 2H), 6.00-5.92 (m, 1H), 5.85 (d, | |
| | J = 10.0 Hz, 1H), 5.16-4.94 (m, 2H), 4.81-4.75 (m, | |
| | 2H), 4.56-4.37 (m, 4H), 4.26-3.99 (m, 3H), 3.90-3.72 | |
| | (m, 6H), 3.67 (d, J = 9.6 Hz, 1H), 3.65-3.43 (m, 4H), | |
| | 3.39-3.32 (m, 2H), 3.12-2.93 (m, 2H), 2.90-2.67 (m, | |
| | 2H), 2.43-2.31 (m, 1H), 2.23-2.14 (m, 1H), 2.11- | |
| | 1.87 (m, 4H), 1.67-1.45 (m, 5H), 1.05 (d, J = 6.4 Hz, | |
| | 3H), 0.94-0.81 (m, 3H). | |
| 139 | 1H-NMR (400 MHz, DMSO-d6) δ 10.08 (s, 1H), 8.98 | Prepared in an analogous manner to |
| | (s, 1H), 8.42 (d, J = 7.6 Hz, 1H), 8.11 (s, 1H), 7.81 (d, | (2S,4R)-1-[(2R)-2-(3-{7-[2-({6- |
| | J = 8.4 Hz, 1H), 7.48-7.41 (m, 4H), 7.40-7.34 (m, 1H), | chloro-4-[(3S)-3-(cyanomethyl)-4- |
| | 7.29 (s, 1H), 7.26-7.20 (m, 1H), 7.19-7.15 (m, 1H), | (prop-2-enoyl)piperazin-1-yl]-8- |
| | 7.07 (d, J = 1.6 Hz, 1H), 7.01-6.77 (m, 1H), 6.21 (d, J = | fluoro-7-(3-hydroxynaphthalen-1- |
| | 16.4 Hz, 1H), 5.85-5.76 (m, 2H), 5.13 (s, 1H), 4.94- | yl)quinazolin-2-yl}oxy)ethyl]-2,7- |
| | 4.86 (m, 3H), 4.46 (s, 2H), 4.35 (t, J = 7.6 Hz, 3H), 4.31- | diazaspiro[3.5]nonan-2-yl}-1,2- |
| | 4.22 (m, 3H), 4.15-4.00 (m, 2H), 3.74-3.67 (m, 3H), | oxazol-5-yl)-3-methylbutanoyl]-4- |
| | 3.60-3.50 (m, 5H), 3.14-3.02 (m, 3H), 2.73-2.63 (m, | hydroxy-N-[(1S)-1-[4-(4-methyl-1,3- |
| | 3H), 2.46 (s, 3H), 2.26-2.16 (m, 1H), 2.10-1.96 (m, | thiazol-5-yl)phenyl]ethyl]pyrrolidine- |
| | 1H), 1.84-1.66 (m, 5H), 1.38 (d, J = 6.8 Hz, 3H), 0.94 | 2-carboxamide |
| | (d, J = 6.4 Hz, 3H), 0.84-0.72 (m, 3H). | |
| 140 | ¹H-NMR (400 MHz, DMSO-d₆) δ 8.98 (s, 1H), 8.44 (d, | Prepared in an analogous manner to |
| | J = 7.6 Hz, 1H), 8.11 (s, 1H), 7.81 (d, J = 8.4 Hz, 1H), | (2S,4R)-1-[(2S)-2-[2-({1-[2-({6- |
| | 7.47-7.39 (m, 3H), 7.37-7.27 (m, 4H), 7.25-7.14 (m, | chloro-4-[(3S)-3-(cyanomethyl)-4- |
| | 2H), 7.07 (d, J = 2.4 Hz, 1H), 6.97-6.79 (m, 1H), 6.25- | (prop-2-enoyl)piperazin-1-yl]-8- |
| | 6.17 (m, 1H), 5.79 (d, J = 9.6 Hz, 1H), 5.25-4.83 (m, | fluoro-7-(3-hydroxynaphthalen-1- |
| | 3H), 4.52 (d, J = 9.6 Hz, 1H), 4.49-4.41 (m, 3H), 4.40- | yl)quinazolin-2- |
| | 4.20 (m, 4H), 4.07 (s, 1H), 3.91 (s, 2H), 3.70-3.53 (m, | yl}oxy)ethyl]piperidin-4- |
| | 7H), 3.05 (s, 2H), 2.82 (t, J = 5.6 Hz, 2H), 2.77-2.70 (m, | yl}methoxy)acetamido]-3,3- |
| | 1H), 2.58 (dd, J = 5.9, 11.2 Hz, 2H), 2.44 (s, 3H), 2.42- | dimethylbutanoyl]-4-hydroxy-N- |
| | 2.35 (m, 2H), 2.09-2.00 (m, 1H), 1.91-1.83 (m, 1H), | [(1S)-1-[4-(4-methyl-1,3-thiazol-5- |
| | 1.80-1.70 (m, 1H), 1.45-1.39 (m, 1H), 1.34 (d, J = 6.8 | yl)phenyl]ethyl]pyrrolidine-2- |
| | Hz, 3H), 0.90 (s, 9H) | carboxamide |
| 141 | ¹H-NMR (400 MHz, DMSO-d₆) δ 8.43 (d, J = 7.6 Hz, | Specific Scheme Provided |
| | 1H), 8.11 (s, 1H), 7.81 (d, J = 8.4 Hz, 1H), 7.50-7.43 | |
| | (m, 4H), 7.40-7.36 (m, 2H), 7.29 (d, J = 2.4 Hz, 1H), | |
| | 7.22 (d, J = 4.0 Hz, 2H), 7.07 (d, J = 2.0 Hz, 1H), 6.37 (d, | |
| | J = 2.0 Hz, 1H), 6.06 (s, 1H), 5.46-5.23 (m, 2H), 5.11 | |
| | (s, 1H), 4.97-4.86 (m, 2H), 4.49 (s, 2H), 4.40-4.33 | |
| | (m, 2H), 4.31-4.26 (m, 2H), 3.98 (d, J = 6.0 Hz, 2H), | |
| | 3.84 (s, 3H), 3.74-3.59 (m, 7H), 3.11-2.96 (m, 3H), | |
| | 2.78 (s, 2H), 2.29-1.95 (m, 5H), 1.81-1.66 (m, 4H), | |
| | 1.48-1.20 (m, 6H), 0.98-0.93 (m, 3H), 0.82-0.75 (m, | |
| | 3H) | |
| 142 | ¹H-NMR (400 MHz, DMSO-d₆) δ 10.26-9.97 (m, 1H), | Prepared in an analogous manner to |
| | 9.04-8.93 (m, 1H), 8.49-8.39 (m, 1H), 8.25-8.05 (m, | (2S,4R)-1-[(2S)-2-[2-({1-[2-({6- |
| | 2H), 7.85-7.75 (m, 1H), 7.48-7.41 (m, 3H), 7.39-7.28 | chloro-4-[(3S)-3-(cyanomethyl)-4- |
| | (m, 4H), 7.24-7.16 (m, 2H), 7.07 (s, 2H), 6.94-6.76 | (prop-2-enoyl)piperazin-1-yl]-8- |
| | (m, 1H), 6.26-6.15 (m, 1H), 5.85-5.73 (m, 1H), 5.19- | fluoro-7-(3-hydroxynaphthalen-1- |
| | 5.10 (m, 1H), 5.04-4.94 (m, 1H), 4.93-4.79 (m, 2H), | yl)quinazolin-2- |
| | 4.56-4.50 (m, 1H), 4.48-4.40 (m, 4H), 4.35 (s, 1H), | yl}oxy)ethyl]piperidin-4- |
| | 4.29-4.22 (m, 2H), 4.13-4.03 (m, 1H), 3.94-3.84 (m, | yl}methoxy)acetamido]-3,3- |
| | 3H), 3.76-3.66 (m, 2H), 3.65-3.54 (m, 8H), 2.85-2.76 | dimethylbutanoyl]-4-hydroxy-N- |
| | (m, 3H), 2.44 (s, 3H), 2.08-2.01 (m, 1H), 1.91-1.84 (m, | [(1S)-1-[4-(4-methyl-1,3-thiazol-5- |
| | 1H), 1.79-1.72 (m, 1H), 1.51-1.26 (m, 5H), 0.91 (s, 9H). | yl)phenyl]ethyl]pyrrolidine-2- |
| | | carboxamide |
| 143 | ¹H-NMR (400 MHz, CD₃OD) δ 8.93-8.82 (m, 1H), 8.52 | Specific Scheme Provided |
| | (s, 1H), 8.09 (s, 1H), 7.75 (d, J = 8.4 Hz, 1H), 7.50-7.33 | |
| | (m, 5H), 7.30-7.13 (m, 3H), 7.04 (d, J = 2.4 Hz, 1H), | |
| | 6.97-6.74 (m, 1H), 6.31 (d, J = 16.8 Hz, 1H), 6.05-5.93 | |
| | (m, 1H), 5.85 (d, J = 9.2 Hz, 1H), 5.05-5.01 (m, 1H), | |
| | 4.75-4.69 (m, 2H), 4.55-4.10 (m, 5H), 4.02-3.66 (m, | |
| | 4H), 3.59 (d, J = 10.0 Hz, 2H), 3.44-3.34 (m, 2H), 3.25- | |
| | 2.98 (m, 6H), 2.97-2.87 (m, 3H), 2.69-2.50 (m, 2H), | |
| | 2.49-2.43 (m, 3H), 2.41-2.30 (m, 1H), 2.22-2.11 (m, | |
| | 1H), 2.06-1.72 (m, 4H), 1.64-1.24 (m, 6H), 1.11-0.98 | |
| | (m, 3H), 0.95-0.80 (m, 3H). | |
| 144 | ¹H-NMR (400 MHz, CD₃OD) δ 8.93-8.78 (m, 1H), 8.50 | Prepared in an analogous manner to |
| | (s, 1H), 8.08 (br s, 1H), 7.75 (d, J = 8.4 Hz, 1H), 7.50- | (2S,4R)-1-[(2R)-2-{3-[({1-[2-({6- |
| | 7.31 (m, 5H), 7.27 (d, J = 2.4 Hz, 1H), 7.25-7.14 (m, | chloro-4-[(3S)-3-(cyanomethyl)-4- |
| | 2H), 7.08-6.99 (m, 1H), 6.96-6.72 (m, 1H), 6.31 (d, J = | (prop-2-enoyl)piperazin-1-yl]-8- |
| | 16.8 Hz, 1H), 6.12-5.96 (m, 1H), 5.85 (d, J = 10.0 Hz, | fluoro-7-(3-hydroxynaphthalen-1- |
| | 1H), 5.01-4.93 (m, 1H), 4.72-4.65 (m, 2H), 4.57-4.09 | yl)quinazolin-2- |
| | (m, 5H), 3.95-3.54 (m, 6H), 3.52-3.38 (m, 2H), 3.30- | yl}oxy)ethyl]piperidin-4- |
| | 3.21 (m, 2H), 3.20-2.97 (m, 4H), 2.95-2.87 (m, 3H), | yl}methyl)(methyl)amino]-1,2- |
| | 2.81-2.61 (m, 2H), 2.51-2.41 (m, 3H), 2.41-2.31 (m, | oxazol-5-yl}-3-methylbutanoyl]-4- |
| | 1H), 2.29-2.15 (m, 1H), 2.10-1.89 (m, 2H), 1.88-1.76 | hydroxy-N-[(1S)-1-[4-(4-methyl-1,3- |
| | (m, 2H), 1.59-1.38 (m, 5H), 1.36-1.27 (m, 1H), 1.05 (d, | thiazol-5-yl)phenyl]ethyl]pyrrolidine- |
| | J = 6.4 Hz, 3H), 0.91 (d, J = 6.8 Hz, 3H). | 2-carboxamide |
| 145 | ¹H-NMR (400 MHz, CD₃OD) δ 8.88-8.83 (m, 1H), 8.03 | Specific Scheme Provided |
| | (s, 1H), 7.74 (d, J = 8.4 Hz, 1H), 7.43-7.36 (m, 5H), | |
| | 7.28-7.17 (m, 3H), 7.07-7.01 (m, 1H), 6.88-6.72 (m, | |
| | 1H), 6.30 (d, J = 16.8 Hz, 1H), 5.84 (d, J = 10.0 Hz, | |
| | 1H), 5.05-4.94 (m, 3H), 4.58-4.36 (m, 7H), 3.90-3.49 | |
| | (m, 6H), 3.21-3.13 (m, 6H), 3.02 (s, 2H), 2.81 (t, J = 5.6 | |
| | Hz, 2H), 2.54 (s, 2H), 2.47 (s, 3H), 2.24-2.15 (m, 1H), | |
| | 1.97-1.88 (m, 1H), 1.81 (s, 4H), 1.59-1.30 (m, 4H), | |
| | 1.05-1.00 (m, 9H). | |
| 146 | ¹H-NMR (400 MHz, DMSO-d₆) δ 8.98 (s, 1H), 8.41 (d, | Prepared in an analogous manner to |
| | J = 7.6 Hz, 1H), 8.02 (s, 1H), 7.81 (d, J = 8.0 Hz, 1H), | (2S,4R)-1-[(2R)-2-(3-{[1-(2-{[6- |
| | 7.46-7.41 (m, 3H), 7.38-7.34 (m, 2H), 7.29 (d, J = 2.4 | chloro-8-fluoro-7-(3- |
| | Hz, 1H), 7.24-7.18 (m, 2H), 7.07 (d, J = 2.4 Hz, 1H), | hydroxynaphthalen-1-yl)-4-[4-(prop- |
| | 6.83 (dd, J = 10.0, 16.4 Hz, 1H), 6.18 (dd, J = 2.0, 16.8 | 2-enoyl)piperazin-1-yl]quinazolin-2- |
| | Hz, 1H), 6.06 (s, 1H), 5.78-5.72 (m, 1H), 5.09 (s, 1H), | yl]oxy}ethyl)piperidin-4- |
| | 4.90 (t, J = 7.2 Hz, 1H), 4.50-4.40 (m, 2H), 4.36 (t, | yl]methoxy}-1,2-oxazol-5-yl)-3- |
| | J = 8.0 Hz, 1H), 4.27 (s, 1H), 4.00-3.91 (m, 6H), 3.90- | methylbutanoyl]-4-hydroxy-N-[(1S)- |
| | 3.74 (m, 5H), 3.69 (dd, J = 4.4, 10.0 Hz, 1H), 3.63 (d, | 1-[4-(4-methyl-1,3-thiazol-5- |
| | J = 10.0 Hz, 1H), 3.00-2.91 (m, 2H), 2.70 (t, J = 6.0 Hz, | yl)phenyl]ethyl]pyrrolidine-2- |
| | 2H), 2.45 (s, 3H), 2.29-2.13 (m, 2H), 2.02 (t, J = 11.6 | carboxamide |
| | Hz, 3H), 1.81-1.74 (m, 1H), 1.67 (d, J = 12.0 Hz, 2H), | |
| | 1.37 (d, J = 7.2 Hz, 3H), 1.31-1.18 (m, 3H), 0.94 (d, | |
| | J = 6.4 Hz, 3H), 0.78 (d, J = 6.8 Hz, 3H). | |
| 147 | ¹H-NMR (400 MHz, CD₃OD) δ 8.89-8.86 (m, 1H), 8.50 | Specific Scheme Provided |
| | (s, 1H), 8.05 (s, 1H), 7.75 (d, J = 8.4 Hz, 1H), 7.48-7.34 | |
| | (m, 5H), 7.29-7.14 (m, 3H), 7.04 (d, J = 2.4 Hz, 1H), | |
| | 6.83 (dd, J = 16.4, 10.4 Hz, 1H), 6.29 (dd, J = 16.4, 1.6 | |
| | Hz, 1H), 6.00-5.92 (m, 1H), 5.82 (dd, J = 10.8, 2.0 Hz, | |
| | 1H), 4.54-4.33 (m, 3H), 4.10-4.02 (m, 6H), 3.97-3.89 | |
| | (m, 4H), 3.83 (dd, J = 10.8, 4.0 Hz, 1H), 3.67 (d, J = | |
| | 10.0 Hz, 1H), 3.61 (d, J = 10.8 Hz, 1H), 3.56-3.35 (m, | |
| | 3H), 3.25-3.17 (m, 2H), 2.81-2.55 (m, 3H), 2.51-2.43 | |
| | (m, 3H), 2.40-2.31 (m, 1H), 2.23-2.13 (m, 1H), 2.03- | |
| | 1.87 (m, 4H), 1.60-1.48 (m, 5H), 1.05 (d, J = 6.4 Hz, | |
| | 3H), 0.88 (d, J = 6.4 Hz, 3H). | |
| 148 | ¹H-NMR (400 MHz, CD₃OD) δ 8.90-8.80 (m, 1H), 8.53 | Specific Scheme Provided |
| | (s, 1H), 8.08 (s, 1H), 7.79-7.71 (m, 1H), 7.47-7.32 (m, | |
| | 5H), 7.29-7.12 (m, 3H), 7.06-7.01 (m, 1H), 6.97-6.72 | |
| | (m, 1H), 6.31 (d, J = 16.8 Hz, 1H), 5.85 (d, J = 10.0 Hz, | |
| | 1H), 4.56-4.39 (m, 4H), 4.26-4.02 (m, 1H), 3.86-3.81 | |
| | (m, 1H), 3.76-3.70 (m, 1H), 3.59-3.37 (m, 3H), 3.14- | |
| | 2.96 (m, 4H), 2.70-2.59 (m, 1H), 2.49-2.41 (m, 3H), | |
| | 2.34-2.17 (m, 5H), 2.03-1.86 (m, 3H), 1.77-1.64 (m, | |
| | 1H), 1.58-1.28 (m, 6H), 1.09-0.93 (m, 9H). | |
| 149 | ¹H-NMR (400 MHz, CD₃OD) δ 8.88-8.82 (m, 1H), | Specific Scheme Provided |
| | 8.57-8.51 (m, 1H), 8.09-8.02 (m, 1H), 7.78-7.71 (m, | |
| | 1H), 7.46-7.33 (m, 5H), 7.30-7.14 (m, 3H), 7.04 (d, J = | |
| | 2.0 Hz, 1H), 6.95-6.72 (m, 1H), 6.38-6.26 (m, 1H), | |
| | 5.90-5.79 (m, 1H), 5.14-4.95 (m, 3H), 4.76-4.72 (m, | |
| | 2H), 4.60-4.55 (m, 2H), 4.53-4.47 (m, 2H), 4.45-4.41 | |
| | (m, 1H), 4.07 (s, 1H), 4.05-3.92 (m, 2H), 3.91-3.81 (m, | |
| | 2H), 3.79-3.67 (m, 2H), 3.62-3.48 (m, 1H), 3.44-3.33 | |
| | (m, 2H), 3.28-3.24 (m, 1H), 3.19-3.08 (m, 2H), 3.07- | |
| | 2.88 (m, 4H), 2.49-2.42 (m, 3H), 2.26-2.16 (m 1H), | |
| | 1.99-1.89 (m, 1H), 1.87-1.73 (m, 2H), 1.62-1.53 (m, | |
| | 2H), 1.46 (d, J = 7.0 Hz, 3H), 1.09 (s, 3H), 1.06-0.98 | |
| | (m, 9H) | |
| 150 | ¹H-NMR (400 MHz, DMSO-d₆) δ 10.12-9.95 (m, 1H), | Specific Scheme Provided |
| | 8.98 (s, 1H), 8.44 (d, J = 8.0 Hz, 1H), 8.18-8.10 (m, | |
| | 1H), 7.81 (d, J = 8.4 Hz, 2H), 7.45-7.35 (m, 5H), 7.30- | |
| | 7.21 (m, 3H), 7.07 (d, J = 2.0 Hz, 1H), 6.21 (d, J = 16.4 | |
| | Hz, 1H), 5.79 (d, J = 10.0 Hz, 1H), 5.20-4.76 (m, 4H), | |
| | 4.56-4.40 (m, 5H), 4.38-4.17 (m, 4H), 3.99 (s, 2H), | |
| | 3.62-3.49 (m, 6H), 2.75-2.74 (s, 8H), 2.45 (s, 3H), | |
| | 2.36-2.29 (m, 2H), 2.10-2.02 (m, 1H), 1.82-1.68 (m, | |
| | 4H), 1.35 (d, J = 6.8 Hz, 3H), 0.92 (s, 9H). | |
| 151 | ¹H-NMR (400 MHz, DMSO-d₆) δ 10.05 (s, 1H), 8.98 | Specific Scheme Provided |
| | (s, 1H), 8.45 (d, J = 7.6 Hz, 1H), 8.11 (s, 1H), 7.81 (d, | |
| | J = 8.4 Hz, 1H), 7.47-7.39 (m, 3H), 7.37-7.32 (m, 2H), | |
| | 7.31-7.26 (m, 2H), 7.22 (d, J = 4.0 Hz, 2H), 7.07 (d, J = | |
| | 2.4 Hz, 1H), 5.42 (dd, J = 18.0, 4.0 Hz, 2H), 5.26-5.09 | |
| | (m, 1H), 4.96-4.76 (m, 2H), 4.53 (d, J = 9.6 Hz, 1H), | |
| | 4.50-4.40 (m, 3H), 4.40-4.25 (m, 3H), 3.91 (s, 2H), | |
| | 3.64-3.48 (m, 2H), 3.33-3.31 (m, 6H), 3.08 (d, J = 6.0 | |
| | Hz, 1H), 3.06-2.95 (m, 3H), 2.78-2.71 (m, 2H), 2.45 (s, | |
| | 3H), 2.13-1.99 (m, 3H), 1.79-1.70 (m, 1H), 1.70-1.52 | |
| | (m, 3H), 1.34 (d, J = 6.8 Hz, 3H), 1.27-1.14 (m, 2H), | |
| | 0.92 (s, 9H). | |
| 152 | ¹H-NMR (400 MHz, CD₃OD) δ 8.92-8.83 (m, 1H), 8.07 | Specific Scheme Provided |
| | (s, 1H), 7.74 (d, J = 8.4 Hz, 1H), 7.48-7.33 (m, 4H), | |
| | 7.28-7.15 (m, 3H), 7.03 (s, 1H), 6.98-6.74 (m, 1H), 6.31 | |
| | (d, J = 17.2 Hz, 1H), 5.90-5.79 (m, 1H), 5.70-5.52 (m, | |
| | 1H), 5.17-4.94 (m, 1H), 4.85-4.80 (m, 1H), 4.69-4.52 | |
| | (m, 5H), 4.50-4.40 (m, 3H), 4.22-4.09 (m, 1H), 3.96- | |
| | 3.80 (m, 4H), 3.77-3.63 (m, 2H), 3.59-3.47 (m, 1H), | |
| | 3.17-2.96 (m, 4H), 2.85-2.70 (m, 1H), 2.59-2.51 (m, | |
| | 1H), 2.47 (s, 3H), 2.26-2.15 (m, 2H), 2.14-2.07 (m, 1H), | |
| | 2.01-1.90 (m, 1H), 1.77-1.59 (m, 3H), 1.57-1.44 (m, | |
| | 3H), 1.39 (d, J = 6.0 Hz, 3H), 1.27-1.12 (m, 2H), 1.01 | |
| | (s, 9H). | |
| 153 | ¹H-NMR (400 MHz, DMSO-d₆) δ 8.99 (s, 1H), 8.42 (d, | Specific Scheme Provided |
| | J = 8.0 Hz, 1H), 8.17 (s, 1H), 8.13-8.09 (m, 1H), 7.82 | |
| | (d, J = 8.8 Hz, 1H), 7.48-7.41 (m, 2H), 7.39-7.33 (m, | |
| | 2H), 7.29 (s, 1H), 7.25-7.15 (m, 2H), 7.10-7.05 (m, 1H), | |
| | 6.97-6.77 (m, 1H), 6.26-6.17 (m, 1H), 6.04 (d, J = 5.6 | |
| | Hz, 1H), 5.89-5.77 (m, 1H), 5.47-5.34 (m, 1H), 5.14- | |
| | 4.97 (m, 1H), 4.96-4.84 (m, 1H), 4.84-4.81 (m, 1H), | |
| | 4.43-4.17 (m, 5H), 4.14-4.03 (m, 1H), 3.97-3.82 (m, | |
| | 3H), 3.73-3.59 (m, 4H), 3.11-2.91 (m, 5H), 2.70-2.61 | |
| | (m, 1H), 2.47 (s, 3H), 2.29-2.15 (m, 2H), 2.07-1.95 (m, | |
| | 4H), 1.77 (m, 1H), 1.72-1.56 (m, 4H), 1.40-1.30 (m, | |
| | 6H), 1.22-1.10 (m, 2H), 0.99-0.91 (m, 3H), 0.83-0.74 | |
| | (m, 3H). | |
| 154 | ¹H-NMR (400 MHz, CD₃OD) δ 8.90-8.82 (m, 1H), 8.52 | Prepared in an analogous manner to |
| | (s, 1H), 8.07 (s, 1H), 7.74 (d, J = 8.0 Hz, 1H), 7.51-7.31 | (2S,4R)-1-[(2R)-2-[3-({1-[2-({6- |
| | (m, 5H), 7.28-7.13 (m, 3H), 7.03 (d, J = 2.4 Hz, 1H), | chloro-4-[(3S)-3-(cyanomethyl)-4- |
| | 6.94-6.72 (m, 1H), 6.31 (d, J = 16.8 Hz, 1H), 6.02-5.96 | (prop-2-enoyl)piperazin-1-yl]-8- |
| | (m, 1H), 5.85 (d, J = 10.0 Hz, 1H), 5.17-4.95 (m, 2H), | fluoro-7-(3-hydroxynaphthalen-1- |
| | 4.74-6.68 (m, 2H), 4.53-4.38 (m, 4H), 4.22-3.43 (m, | yl)quinazolin-2- |
| | 8H), 3.37-3.33 (m, 1H), 3.25-2.92 (m, 5H), 2.61-2.15 | yl}oxy)ethyl]piperidin-4- |
| | (m, 8H), 2.08-1.80 (m, 4H), 1.62-1.36 (m, 5H), 1.08- | yl}methoxy)-1,2-oxazol-5-yl]-3- |
| | 0.93 (m, 3H), 0.93-0.81 (m, 3H). | methylbutanoyl]-4-hydroxy-N-[(1S)- |
| | | 1-[4-(4-methyl-1,3-thiazol-5- |
| | | yl)phenyl]ethyl]pyrrolidine-2- |
| | | carboxamide |
| 155 | ¹H-NMR (400 MHz, CD₃OD) δ 8.91-8.85 (m, 1H), 8.05 | Prepared similarly to (2S,4R)-1- |
| | (s, 1H), 7.74 (d, J = 8.4 Hz, 1H), 7.49-7.38 (m, 4H), | [(2R)-2-[3-({1-[2-({6-chloro-4-[(3S)- |
| | 7.29-7.11 (m, 3H), 7.07-7.00 (m, 1H), 6.00-5.87 (m, | 3-(cyanomethyl)-4-(2-fluoroprop-2- |
| | 1H), 5.63 (d, J = 3.2 Hz, 1H), 5.48-4.90 (m, 2H), 4.68- | enoyl)piperazin-1-yl]-8-fluoro-7-(3- |
| | 4.39 (m, 8H), 3.98-3.57 (m, 8H), 3.22-2.93 (m, 3H), | hydroxynaphthalen-1-yl)quinazolin- |
| | 2.86-2.71 (m, 2H), 2.59-2.27 (m, 5H), 2.22-1.91 (m, | 2-yl}oxy)ethyl]piperidin-4- |
| | 3H), 1.71 (t, J = 12.0 Hz, 3H), 1.59-1.49 (m, 3H), 1.38 | yl}methoxy)-1,2-oxazol-5-yl]-3- |
| | (d, J = 6.4 Hz, 3H), 1.20 (d, J = 11.2 Hz, 2H), 1.03 (dd, | methylbutanoyl]-4-hydroxy-N-[(1S)- |
| | J = 6.4, 2.8 Hz, 3H), 0.89-0.80 (m, 3H). | 1-[4-(4-methyl-1,3-thiazol-5- |
| | | yl)phenyl]ethyl]pyrrolidine-2- |
| | | carboxamide |
| 156 | ¹H-NMR (400 MHz, CD₃OD) δ 8.05 (s, 1H), 7.75 (d, | Prepared in an analogous manner to |
| | J = 8.0 Hz, 1H), 7.51-7.36 (m, 6H), 7.30-7.14 (m, 3H), | (2S,4R)-1-[(2R)-2-[3-({1-[2-({6- |
| | 7.03 (d, J = 2.4 Hz, 1H), 6.37-6.30 (m, 1H), 6.01-5.90 | chloro-4-[(3S)-3-(cyanomethyl)-4-(2- |
| | (m, 1H), 5.47-5.22 (m, 2H), 5.08-4.99 (m, 1H), 4.56- | fluoroprop-2-enoyl)piperazin-1-yl]-8- |
| | 4.35 (m, 5H), 4.03 (d, J = 5.2 Hz, 2H), 3.90-3.44 (m, | fluoro-7-(3-hydroxynaphthalen-1- |
| | 10H), 3.23-3.00 (m, 4H), 2.95-2.84 (m, 2H), 2.46-1.71 | yl)quinazolin-2- |
| | (m, 9H), 1.63-1.26 (m, 6H), 1.05 (d, J = 6.4 Hz, 3H), | yl}oxy)ethyl]piperidin-4- |
| | 0.94-0.81 (m, 3H). | yl}methoxy)-1,2-oxazol-5-yl]-3- |
| | | methylbutanoyl]-4-hydroxy-N-[(1S)- |
| | | 1-[4-(1-methyl-1H-pyrazol-5- |
| | | yl)phenyl]ethyl]pyrrolidine-2- |
| | | carboxamide |
| 157 | ¹H-NMR (400 MHz, CD₃OD) δ 8.48 (s, 1H), 8.10 (s, | Prepared in an analogous manner to |
| | 1H), 7.76 (d, J = 8.0 Hz, 1H), 7.52-7.37 (m, 6H), 7.31- | (2S,4R)-1-[(2R)-2-[3-({1-[2-({6- |
| | 7.14 (m, 3H), 7.04 (d, J = 2.4 Hz, 1H), 6.98-6.71 (m, | chloro-4-[(3S)-3-(cyanomethyl)-4- |
| | 1H), 6.39-6.25 (m, 2H), 6.04-5.92 (m, 1H), 5.91-5.79 | (prop-2-enoyl)piperazin-1-yl]-8- |
| | (m, 1H), 5.16-5.00 (m, 1H), 4.80-4.75 (m, 3H), 4.53- | fluoro-7-(3-hydroxynaphthalen-1- |
| | 4.37 (m, 2H), 4.23-4.03 (m, 3H), 4.00-3.43 (m, 13H), | yl)quinazolin-2- |
| | 3.22-2.60 (m, 5H), 2.44-1.87 (m, 7H), 1.68-1.49 (m, | yl}oxy)ethyl]piperidin-4- |
| | 5H), 1.05 (d, J = 6.8 Hz, 3H), 0.93-0.84 (m, 3H). | yl}methoxy)-1,2-oxazol-5-yl]-3- |
| | | methylbutanoyl]-4-hydroxy-N-[(IS)- |
| | | 1-[4-(1-methyl-1H-pyrazol-5- |
| | | yl)phenyl]ethyl]pyrrolidine-2- |
| | | carboxamide |
| 158 | ¹H-NMR (400 MHz, CD₃OD) δ 8.47 (s, 1H), 8.08 (s, | Prepared in an analogous manner to |
| | 1H), 7.74 (d, J = 8.0 Hz, 1H), 7.47 (d, J = 2.0 Hz, 1H) | (2S,4R)-1-[(2R)-2-[3-({1-[2-({6- |
| | 7.33-7.43 (m, 5H), 7.17-7.29 (m, 3H), 7.03 (d, J = 4.8 | chloro-4-[(3S)-3-(cyanomethyl)-4-(2- |
| | Hz, 1H), 6.27-6.35 (m, 1H), 5.99-6.05 (m, 1H), 5.26- | fluoroprop-2-enoyl)piperazin-1-yl]-8- |
| | 5.47 (m, 2H), 4.34-4.61 (m, 5H), 3.91-4.16 (m, 3H), | fluoro-7-(3-hydroxynaphthalen-1- |
| | 3.35-3.89 (m, 15H), 3.08 (d, J = 5.2 Hz, 2H), 2.90 (t, J = | yl)quinazolin-2- |
| | 12.0 Hz, 2H), 2.16-2.44 (m, 2H), 1.85-2.09 (m, 5H), | yl}oxy)ethyl]piperidin-4- |
| | 1.52-1.71 (m, 3H), 1.47 (d, J = 7.2 Hz, 3H), 1.05 (d, J = | yl}methoxy)-1,2-oxazol-5-yl]-3- |
| | 6.8 Hz, 3H), 0.90 (d, J = 6.8 Hz, 3H). | methylbutanoyl]-4-hydroxy-N-[(1S)- |
| | | 1-[4-(1-methyl-1H-pyrazol-5- |
| | | yl)phenyl]ethyl]pyrrolidine-2- |
| | | carboxamide |
| 159 | ¹H-NMR (400 MHz, CD₃OD) δ 8.48 (s, 1H), 8.09 (s, | Prepared in an analogous manner to |
| | 1H), 7.74 (d, J = 8.0, Hz, 1H), 7.36-7.48 (m, 6H), 7.18- | (2S,4R)-1-[(2R)-2-[3-({1-[2-({6- |
| | 7.28 (m, 3H), 7.00-7.06 (m, 1H), 6.82 (d, J = 11.6 Hz, | chloro-4-[(3S)-3-(cyanomethyl)-4- |
| | 1H), 6.26-6.34 (m, 2H), 6.01 (s, 1H), 5.87-5.81 (m, 1H), | (prop-2-enoyl)piperazin-1-yl]-8- |
| | 5.04-5.16 (m, 1H), 4.51-4.61 (m, 3H), 4.39-4.50 (m, | fluoro-7-(3-hydroxynaphthalen-1- |
| | 2H), 3.98-4.18 (m, 3H), 3.36-3.84 (m, 13H), 2.68-3.17 | yl)quinazolin-2- |
| | (m, 5H), 1.90-2.41 (m, 7H), 1.44-1.60 (m, 5H), 1.05 (d, | yl}oxy)ethyl]piperidin-4- |
| | J = 6.4 Hz, 3H), 0.90 (d, J = 6.4 Hz, 3H). | yl}methoxy)-1,2-oxazol-5-yl]-3- |
| | | methylbutanoyl]-4-hydroxy-N-[(1S)- |
| | | 1-[4-(1-methyl-1H-pyrazol-5- |
| | | yl)phenyl]ethyl]pyrrolidine-2- |
| | | carboxamide |
| 160 | ¹H-NMR (400 MHz, CD₃OD) δ 8.51 (s, 1H), 8.08 (br s, | Prepared in an analogous manner to |
| | 1H), 7.80-7.69 (m, 1H), 7.66-7.50 (m, 2H), 7.46- | (2S,4R)-1-[(2R)-2-[3-({1-[2-({6- |
| | 7.35 (m, 3H), 7.30-7.14 (m, 3H), 7.07-7.00 (m, 1H), | chloro-4-[(3S)-3-(cyanomethyl)-4- |
| | 6.99-6.69 (m, 1H), 6.37-6.25 (m, 1H), 6.04-5.93 (m, | (prop-2-enoyl)piperazin-1-yl]-8- |
| | 1H), 5.90-5.75 (m, 1H), 5.13-4.93 (m, 2H), 4.80- | fluoro-7-(3-hydroxynaphthalen-1- |
| | 4.70 (m, 3H), 4.58-4.38 (m, 5H), 4.09-3.99 (m, 2H), | yl)quinazolin-2- |
| | 3.91-3.83 (m, 1H), 3.79-3.62 (m, 4H), 3.50-3.40 (m, | yl}oxy)ethyl]piperidin-4- |
| | 2H), 3.29-3.21 (m, 2H), 3.08-2.96 (m, 2H), 2.79- | yl}methoxy)-1,2-oxazol-5-yl]-3- |
| | 2.60 (m, 2H), 2.42-2.30 (m, 1H), 2.26-2.18 (m, 1H), | methylbutanoyl]-4-hydroxy-N-[(1S)- |
| | 2.08-1.84 (m, 4H), 1.63-1.49 (m, 2H), 1.44 (d, J = 6.8 | 1-[4-(4-methyl-1,3-thiazol-5- |
| | Hz, 3H), 1.04 (d, J = 6.8 Hz, 3H), 0.95-0.86 (m, 3H). | yl)phenyl]ethyl]pyrrolidine-2- |
| | | carboxamide |
| 161 | ¹H-NMR (400 MHz, CD₃OD) δ 8.51 (s, 1H), 8.08 (s, | Prepared in an analogous manner to |
| | 1H), 7.75 (d, J = 8.4 Hz, 1H), 7.71-7.63 (m, 2H), 7.51- | (2S,4R)-1-[(2R)-2-[3-({1-[2-({6- |
| | 7.37 (m, 3H), 7.27 (d, J = 2.3 Hz, 1H), 7.25-7.14 (m, | chloro-4-[(3S)-3-(cyanomethyl)-4- |
| | 2H), 7.04 (d, J = 2.4 Hz, 1H), 6.92-6.72 (m, 1H), 6.37- | (prop-2-enoyl)piperazin-1-yl]-8- |
| | 6.26 (m, 1H), 6.00-5.90 (m, 1H), 5.89-5.80 (m, 1H), | fluoro-7-(3-hydroxynaphthalen-1- |
| | 5.14-4.96 (m, 1H), 4.75-4.72 (m, 2H), 4.54-4.36 (m, | yl)quinazolin-2- |
| | 5H), 4.21-4.00 (m, 3H), 3.91-3.79 (m, 2H), 3.78- | yl}oxy)ethyl]piperidin-4- |
| | 3.70 (m, 1H), 3.66 (d, J = 9.9 Hz, 1H), 3.63-3.57 (m, | yl}methoxy)-1,2-oxazol-5-yl]-3- |
| | 1H), 3.55-3.37 (m, 3H), 3.29-3.18 (m, 2H), 3.09- | methylbutanoyl]-4-hydroxy-N-[(1S)- |
| | 2.92 (m, 2H), 2.76-2.60 (m, 2H), 2.36 (qd, J = 6.7, | 1-[4-(4-methyl-1,3-thiazol-5- |
| | 16.6 Hz, 1H), 2.22-2.12 (m, 1H), 2.07-1.85 (m, 4H), | yl)phenyl]ethyl]pyrrolidine-2- |
| | 1.62-1.53 (m, 2H), 1.49 (d, J = 7.0 Hz, 3H), 1.06- | carboxamide |
| | 0.99 (m, 3H), 0.91-0.83 (m, 3H). | |
| 162 | ¹H-NMR (400 MHz, DMSO-d₆) δ 9.02-8.96 (m, 1H), | Prepared in an analogous manner to |
| | 8.42 (d, J = 7.6 Hz, 1H), 8.18 (s, 1H), 8.11 (s, 1H), 7.81 | (2S,4R)-1-[(2S)-2-[2-({1-[2-({6- |
| | (d, J = 8.4 Hz, 1H), 7.47-7.42 (m, 3H), 7.40-7.35 (m, | chloro-4-[(3S)-3-(cyanomethyl)-4- |
| | 2H), 7.30-7.26 (m, 2H), 7.23 (d, J = 3.6 Hz, 2H), 7.07 | (prop-2-enoyl)piperazin-1-yl]-8- |
| | (d, J = 2.4 Hz, 1H), 6.99-6.79 (m, 1H), 6.22 (dd, J = | fluoro-7-(3-hydroxynaphthalen-1- |
| | 16.8, 1.6 Hz, 1H), 5.80 (d, J = 9.2 Hz, 1H), 5.35 (s, 1H), | yl)quinazolin-2- |
| | 5.03-4.83 (m, 2H), 4.51-4.45 (m, 4H), 4.36-4.33 (m, | yl}oxy)ethyl]piperidin-4- |
| | 2H), 4.28-4.18 (m, 3H), 4.09 (dd, J = 11.6, 2.0 Hz, 1H), | yl}methoxy)acetamido]-3,3- |
| | 3.90 (s, 2H), 3.87-3.82 (m, 3H), 3.09-2.93 (m, 6H), | dimethylbutanoyl]-4-hydroxy-N- |
| | 2.74-2.66 (m, 1H), 2.46 (s, 3H), 2.03 (t, J = 11.6 Hz, | [(1S)-1-[4-(4-methyl-1,3-thiazol-5- |
| | 3H), 1.67-1.54 (m, 5H), 1.36 (d, J = 7.2 Hz, 3H), 1.21- | yl)phenyl]ethyl]pyrrolidine-2- |
| | 1.89 (m, 2H), 0.94 (s, 9H). | carboxamide |
| 163 | ¹H-NMR (400 MHz, DMSO-d₆) δ 8.98 (s, 1H), 8.45 (d, | Specific Scheme Provided |
| | J = 7.6 Hz, 1H), 8.11 (s, 1H), 7.81 (d, J = 8.4 Hz, 1H), | |
| | 7.47-7.39 (m, 3H), 7.38-7.32 (m, 2H), 7.32-7.25 (m, | |
| | 2H), 7.22 (d, J = 3.6 Hz, 2H), 7.07 (d, J = 2.4 Hz, 1H), | |
| | 6.98-6.79 (m, 1H), 6.21 (d, J = 16.4 Hz, 1H), 5.79 (d, | |
| | J = 10.8 Hz, 1H), 4.99 (s, 1H), 4.91-4.83 (m, 1H), 4.53 | |
| | (d, J = 9.6 Hz, 1H), 4.50-4.42 (m, 3H), 4.41-4.20 (m, | |
| | 4H), 4.07 (s, 1H), 3.90 (s, 2H), 3.79-3.55 (m, 8H), 3.05- | |
| | 2.91 (m, 4H), 2.72 (s, 2H), 2.45 (s, 3H), 2.09-1.99 (m, | |
| | 3H), 1.79-1.71 (m, 1H), 1.68-1.53 (m, 3H), 1.34 (d, J = | |
| | 6.8 Hz, 3H), 1.25-1.14 (m, 2H), 0.92 (s, 9H). | |
| 164 | ¹H-NMR (400 MHz, CDCl₃) δ 8.66 (s, 1H), 8.42 (s, | Specific Scheme Provided |
| | 1H), 7.94-7.85 (m, 1H), 7.85-7.72 (m, 2H), 7.60- | |
| | 7.49 (m, 2H), 7.43-7.31 (m, 6H), 7.27-7.26 (m, 1H), | |
| | 7.25-7.15 (m, 2H), 6.32-6.23 (m, 1H), 5.12-5.03 (m, | |
| | 1H), 4.99-4.82 (m, 1H), 4.77-4.65 (m, 3H), 4.60- | |
| | 4.55 (m, 1H), 4.53-4.47 (m, 1H), 4.42-4.30 (m, 1H), | |
| | 4.29-4.20 (m, 1H), 4.09-3.96 (m, 2H), 3.92-3.84 (m, | |
| | 1H), 3.79-3.70 (m, 3H), 3.65-3.58 (m, 1H), 3.52- | |
| | 3.04 (m, 13H), 2.65-2.59 (m, 1H), 2.56-2.47 (m, 6H), | |
| | 2.45-2.36 (m, 1H), 2.14-2.04 (m, 1H), 1.89-1.67 (m, | |
| | 3H), 1.64-1.50 (m, 2H), 1.46 (t, J = 6.4 Hz, 3H), 1.10- | |
| | 0.94 (m, 9H). | |
| 165 | ¹H-NMR (400 MHz, CDCl₃) 8.67 (s, 1H), 8.42 (s, 1H), | Specific Scheme Provided |
| | 7.90 (dd, J = 8.4, 3.6 Hz, 1H), 7.81-7.73 (m, 2H), 7.59- | |
| | 7.49 (m, 2H), 7.43-7.31 (m, 6H), 7.30-7.25 (m, 2H), | |
| | 7.27-7.15 (m, 2H), 6.30-6.21 (m, 1H), 5.14-5.00 (m, | |
| | 1H), 4.79-4.65 (m, 3H), 4.62-4.46 (m, 2H), 4.40- | |
| | 4.19 (m, 2H), 4.10-3.96 (m, 2H), 3.92-3.84 (m, 1H), | |
| | 3.64-3.57 (m, 1H), 3.49-3.05 (m, 15H), 2.68-2.55 | |
| | (m, 2H), 2.51 (d, J = 2.4 Hz, 3H), 2.45-2.39 (m, 2H), | |
| | 2.34 (s, 6H), 1.89-1.64 (m, 4H), 1.60-1.49 (m, 2H), | |
| | 1.46 (dd, J = 8.4, 7.2 Hz, 3H), 1.04 (s, 9H). | |
| 166 | ¹H-NMR (400 MHz, DMSO-d₆) δ 10.05 (s, 1H), 8.98 | Prepared in an analogous manner to |
| | (s, 1H), 8.42 (d, J = 7.6 Hz, 1H), 8.11 (s, 1H), 7.81 (d, | (2S,4R)-1-[(2R)-2-[3-({1-[2-({6- |
| | J = 8.4 Hz, 1H), 7.47-7.40 (m, 3H), 7.39-7.34 (m, 2H), | chloro-4-[(3S)-3-(cyanomethyl)-4-(2- |
| | 7.29 (d, J = 2.0 Hz, 1H), 7.25-7.19 (m, 1H), 7.18-7.13 | fluoroprop-2-enoyl)piperazin-1-yl]-8- |
| | (m, 1H), 7.07 (d, J = 2.4 Hz, 1H), 6.06 (s, 1H), 5.42 (dd, | fluoro-7-(3-hydroxynaphthalen-1- |
| | J = 18.0, 4.0 Hz, 2H), 5.10 (d, J = 3.6 Hz, 1H), 4.98- | yl)quinazolin-2- |
| | 4.76 (m, 2H), 4.47 (d, J = 5.6 Hz, 2H), 4.40-4.23 (m, | yl}oxy)ethyl]piperidin-4- |
| | 4H), 3.98 (d, J = 6.0 Hz, 2H), 3.72-3.48 (m, 4H), 3.43 | yl}methoxy)-1,2-oxazol-5-yl]-3- |
| | (m, 4H), 3.13-2.95 (m, 3H), 2.73 (s, 2H), 2.45 (s, 3H), | methylbutanoyl]-4-hydroxy-N-[(1S)- |
| | 2.26-2.16 (m, 1H), 2.06-1.96 (m, 2H), 1.83-1.62 (m, | 1-[4-(4-methyl-1,3-thiazol-5- |
| | 4H), 1.37 (d, J = 7.2 Hz, 2H), 1.33-1.18 (m, 4H), 0.98- | yl)phenyl]ethyl]pyrrolidine-2- |
| | 0.92 (m, 3H), 0.82-0.75 (m, 3H). | carboxamide |
| 167 | ¹H-NMR (400 MHz, DMSO-d₆) δ 8.98 (s, 1H), 8.41 (d, | Specific Scheme Provided |
| | J = 7.2 Hz, 1H), 8.15 (s, 1H), 8.11 (s, 1H), 7.81 (d, J = | |
| | 8.4 Hz, 1H), 7.47-7.41 (m, 4H), 7.39-7.33 (m, 2H), 7.29 | |
| | (d, J = 2.4 Hz, 1H), 7.22 (d, J = 3.6 Hz, 2H), 7.06 (d, J = | |
| | 2.4 Hz, 1H), 6.06 (s, 1H), 5.49-5.09 (m, 3H), 4.90 (t, J = | |
| | 7.2 Hz, 2H), 4.74-4.17 (m, 8H), 3.98 (d, J = 5.6 Hz, | |
| | 4H), 3.75-3.55 (m, 6H), 3.11-2.86 (m, 3H), 2.72 (s, 2H), | |
| | 2.45 (s, 3H), 2.33 (s, 1H), 2.03 (s, 2H), 1.68 (d, J = 11.6 | |
| | Hz, 3H), 1.40-1.25 (m, 5H), 0.94 (d, J = 6.4 Hz, 3H), | |
| | 0.78 (d, J = 6.8 Hz, 3H). | |
| 168 | ¹H-NMR (400 MHz, DMSO-d6) δ 10.06 (s, 1H), 8.99 | Prepared in an analogous manner to |
| | (s, 1H), 8.43 (d, J = 7.6 Hz, 1H), 8.13 (d, J = 7.6 Hz, | (2S,4R)-1-[(2R)-2-[3-({1-[2-({6- |
| | 1H), 7.82 (d, J = 7.6 Hz, 1H), 7.44 (d, J = 8.0 Hz, 3H), | chloro-4-[(3S)-3-(cyanomethyl)-4-(2- |
| | 7.39-7.34 (m, 2H), 7.30 (s, 1H), 7.24-7.14 (m, 2H), 7.07 | fluoroprop-2-enoyl)piperazin-1-yl]-8- |
| | (s, 1H), 6.07 (s, 1H), 5.43 (d, J = 18.0 Hz, 1H), 5.11 (d, | fluoro-7-(3-hydroxynaphthalen-1- |
| | J = 3.6 Hz, 1H), 5.00-4.79 (m, 2H), 4.50 (s, 2H), 4.36 (t, | yl)quinazolin-2- |
| | J = 8.0 Hz, 2H), 4.32-4.25 (m, 2H), 3.99 (d, J = 4.8 Hz, | yl}oxy)ethyl]piperidin-4- |
| | 2H), 3.75-3.62 (m, 3H), 3.60-3.44 (m, 6H), 3.12-3.03 | yl}methoxy)-1,2-oxazol-5-yl]-3- |
| | (m, 2H), 2.82 (s, 2H), 2.46 (s, 3H), 2.23 (m, 3H), 2.04 | methylbutanoyl]-4-hydroxy-N-[(1S)- |
| | (d, J = 8.0 Hz, 1H), 1.84-1.66 (m, 4H), 1.45-1.25 (m, | 1-[4-(4-methyl-1,3-thiazol-5- |
| | 6H), 0.95 (d, J = 6.4 Hz, 3H), 0.79 (d, J = 6.4 Hz, 3H). | yl)phenyl]ethyl]pyrrolidine-2- |
| | | carboxamide |
| 169 | ¹H-NMR (400 MHz, DMSO-d6) δ 10.07 (d, J = 2.8 Hz, | Prepared in an analogous manner to |
| | 1H), 8.99 (s, 1H), 8.44 (d, J = 7.6 Hz, 1H), 8.15-8.13 | (2S,4R)-1-[(2R)-2-[3-({1-[2-({6- |
| | (m, 1H), 7.82 (d, J = 8.4 Hz, 1H), 7.44 (d, J = 8.0 Hz, | chloro-4-[(3S)-3-(cyanomethyl)-4- |
| | 3H), 7.39-7.35 (m, 2H), 7.30 (d, J = 2.0 Hz, 1H), 7.25- | (prop-2-enoyl)piperazin-1-yl]-8- |
| | 7.14 (m, 2H), 7.08 (d, J = 2.4 Hz, 1H), 6.99-6.78 (m, | fluoro-7-(3-hydroxynaphthalen-1- |
| | 1H), 6.22 (d, J = 16.4 Hz, 1H), 6.08 (s, 1H), 5.80 (d, J = | yl)quinazolin-2- |
| | 9.2 Hz, 1H), 5.12 (d, J = 3.2 Hz, 1H), 5.03-4.87 (m, | yl}oxy)ethyl]piperidin-4- |
| | 2H), 4.53 (s, 2H), 4.36 (t, J = 8.0 Hz, 2H), 4.31-4.23 (m, | yl}methoxy)-1,2-oxazol-5-yl]-3- |
| | 2H), 4.09 (d, J = 12.0 Hz, 1H), 4.00 (d, J = 5.6 Hz, 2H), | methylbutanoyl]-4-hydroxy-N-[(1S)- |
| | 3.87 (s, 1H), 3.75-3.62 (m, 3H), 3.57 (d, J = 12 Hz, 1H), | 1-[4-(4-methyl-1,3-thiazol-5- |
| | 3.44 (m, 4H), 3.07 (s, 2H), 2.46 (s, 3H), 2.32-2.06 (m, | yl)phenyl]ethyl]pyrrolidine-2- |
| | 3H), 2.06-1.99 (m, 1H), 1.77 (m, 4H), 1.54-1.23 (m, | carboxamide |
| | 6H), 0.98-0.93 (m, 3H), 0.84-0.77 (m, 3H). | |
| 170 | ¹H-NMR (400 MHz, CD₃OD) δ 8.87 (s, 1H), 8.46 (brs, | Specific Scheme Provided |
| | 1H), 8.09 (s, 1H), 7.76 (d, J = 7.6 Hz, 1H), 7.59-7.30 | |
| | (m, 6H), 7.28 (s, 1H), 7.25-7.15 (m, 2H), 7.05 (s, 1H), | |
| | 5.44-5.32 (m, 2H), 5.05-4.93 (m, 2H), 4.81-4.70 (m, | |
| | 3H), 4.60-4.40 (m, 4H), 4.35-4.10 (m, 1H), 4.06 (s, | |
| | 2H), 3.90-3.60 (m, 10H), 3.55-3.35 (m, 4H), 3.20- | |
| | 3.00 (m, 4H), 2.46 (d, J = 2.4 Hz, 3H), 2.30-2.15 (m, | |
| | 1H), 2.10-1.85 (m, 5H), 1.57, 1.48 (d, J = 6.8 Hz, 3H), | |
| | 1.04 (s, 9H). | |
| 171 | ¹H-NMR (400 MHz, CD₃OD) δ 9.11-8.75 (s, 1H), | Prepared in an analogous manner to |
| | 8.58-8.37 (s, 1H), 8.07 (s, 1H), 7.75 (d, J = 7.6 Hz, | (2S,4R)-1-[(2R)-2-[3-({1-[2-({6- |
| | 1H), 7.49-7.31 (m, 6H), 7.29-7.14 (m, 3H), 7.08- | chloro-4-[(3S)-3-(cyanomethyl)-4-(2- |
| | 6.99 (m, 1H), 6.08-5.91 (m, 1H), 5.46-5.27 (m, 2H), | fluoroprop-2-enoyl)piperazin-1-yl]-8- |
| | 5.01-4.94 (m, 2H), 4.73 (s, 2H), 4.61-4.12 (m, 5H), | fluoro-7-(3-hydroxynaphthalen-1- |
| | 4.09-3.97 (m, 2H), 3.87-3.54 (m, 6H), 3.20-3.00 (m, | yl)quinazolin-2- |
| | 5H), 2.62-2.48 (m, 2H), 2.46 (s, 3H), 2.40-2.31 (m, | yl}oxy)ethyl]piperidin-4- |
| | 1H), 2.26-2.17 (m, 1H), 2.00-1.82 (m, 4H), 1.56- | yl}methoxy)-1,2-oxazol-5-yl]-3- |
| | 1.42 (m, 5H), 1.05 (d, J = 6.4 Hz, 3H), 0.91 (d, J = 6.8 | methylbutanoyl]-4-hydroxy-N-[(1S)- |
| | Hz, 3H). | 1-[4-(4-methyl-1,3-thiazol-5- |
| | | yl)phenyl]ethyl]pyrrolidine-2- |
| | | carboxamide |
| 172 | ¹H-NMR (400 MHz, CD₃OD) δ 8.91-8.83 (s, 1H), | Prepared in an analogous manner to |
| | 8.54-8.43 (s, 1H), 8.09 (s, 1H), 7.78-7.71 (m, 1H), | (2S,4R)-1-[(2R)-2-[3-({1-[2-({6- |
| | 7.49-7.32 (m, 6H), 7.26 (s, 1H), 7.24-7.15 (m, 2H), | chloro-4-[(3S)-3-(cyanomethyl)-4- |
| | 7.03 (dd, J = 2.4, 4.4 Hz, 1H), 6.31 (d, J = 16.8 Hz, 1H), | (prop-2-enoyl)piperazin-1-yl]-8- |
| | 6.01 (s, 1H), 5.89-5.79 (m, 1H), 5.14-5.07 (m, 1H), | fluoro-7-(3-hydroxynaphthalen-1- |
| | 5.00-4.94 (m, 2H), 4.76 (d, J = 2.0 Hz, 2H), 4.62- | yl)quinazolin-2- |
| | 4.39 (m, 4H), 4.22-3.98 (m, 2H), 3.94-3.81 (m, 1H), | yl}oxy)ethyl]piperidin-4- |
| | 3.79-3.60 (m, 4H), 3.54-3.40 (m, 2H), 3.20-2.92 (m, | yl}methoxy)-1,2-oxazol-5-yl]-3- |
| | 3H), 2.86-2.57 (m, 2H), 2.46 (s, 3H), 2.42-2.14 (m, | methylbutanoyl]-4-hydroxy-N-[(1S)- |
| | 2H), 2.11-1.73 (m, 5H), 1.47 (d, J = 7.2 Hz, 6H), 1.05 | 1-[4-(4-methyl-1,3-thiazol-5- |
| | (d, J = 6.8 Hz, 3H), 0.91 (d, J = 6.4 Hz, 3H). | yl)phenyl]ethyl]pyrrolidine-2- |
| | | carboxamide |
| 173 | ¹H-NMR (400 MHz, CD₃OD) δ 8.90-8.85 (m, 1H), | Specific Scheme Provided |
| | 8.47 (s, 1H), 8.09 (s, 1H), 7.75 (d, J = 8.4 Hz, 1H), 7.47- | |
| | 7.35 (m, 5H), 7.27 (d, J = 2.0 Hz, 1H), 7.24-7.14 (m, | |
| | 2H), 7.03 (d, J = 2.4 Hz, 1H), 6.31 (d, J = 16.4 Hz, 1H), | |
| | 5.99 (s, 1H), 5.89-5.78 (m, 1H), 5.14-4.97 (m, 2H), | |
| | 4.81-4.72 (m, 3H), 4.56-4.46 (m, 3H), 4.46-4.37 (m, | |
| | 1H), 4.22-4.13 (m, 1H), 4.11-4.03 (m, 2H), 3.97- | |
| | 3.80 (m, 3H), 3.69-3.65 (m, 2H), 3.42-3.33 (m, 2H), | |
| | 3.07-2.97 (m, 2H), 2.92-2.72 (m, 2H), 2.48 (s, 3H), | |
| | 2.43-2.28 (m, 1H), 2.23-2.14 (m, 1H), 2.12-1.91 (m, | |
| | 4H), 1.66-1.57 (m, 2H), 1.52 (d, J = 7.2 Hz, 3H), 1.05 | |
| | (d, J = 6.4 Hz, 3H), 0.95-0.83 (m, 3H). | |

### Exemplary Embodiments

In any aspect or embodiment described herein, the hetero-bifunctional compound has the chemical structure:

PTM-L-VLM,

or a pharmaceutically acceptable salt or solvate thereof,

wherein:
(a) the PTM is a small molecule Kirsten ras sarcoma protein (KRas) targeting moiety that binds to KRas^{G12C}, and is represented by the chemical structure:

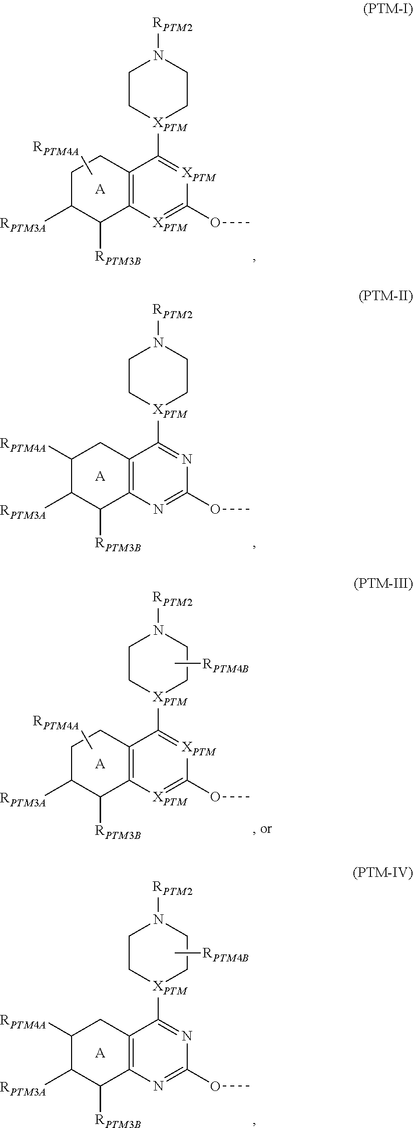

wherein:

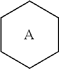

is an 6-membered aryl, 6-membered heteroaryl, or a 6-membered heterocycloalkyl, each optionally substituted with 1 or 2 halogens (e.g., Cl, F, or Br);
R_{PTM2} is —C(═O)C₂-C₄alkenyl, optionally substituted by a methyl or halogen (e.g., Cl, F, Br);
R_{PTM3A} is H, phenyl, or naphthalene, each optionally substituted by 1, 2, or 3 groups independently selected from OH, halogen (e.g., F, Cl, Br), or a linear or branched C₁-C₃ alkyl (e.g., methyl or ethyl);
R_{PTM3B} is H, halogen (e.g., Cl, F, Br), or —O—R_{PTM3C}, wherein R_{PTM3C} is an indazole

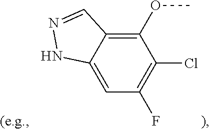

wherein R_{PTM3B} is optionally substituted by 1, 2, or 3 groups independently selected from OH, halogen (e.g., F, Cl, Br), or a linear or branched C₁-C₃ alkyl (e.g., methyl or ethyl);
R_{PTM4A} of PTM-I or PTM=III is absent (or H), or 1 or 2 independently selected halogen (e.g., Cl, F, Br);
R_{PTM4A} of PTM-II and PTM-IV is absent (or H) or a halogen (e.g., Cl, F, Br);
R_{PTM4B} is (1) absent (or H), (2) —CH₂—CH₂—CN or —CH₂—CN, or (3) 1 or 2 independently selected C₁-C₃ alkyl (e.g., methyl or ethyl); and
each X_{PTM} is individually a CH or N;
(b) the VLM is a small molecule E3 ubiquitin ligase binding moiety that binds a Von Hippel-Lindau (VHL) E3 ubiquitin ligase, and is represented by the chemical structure:

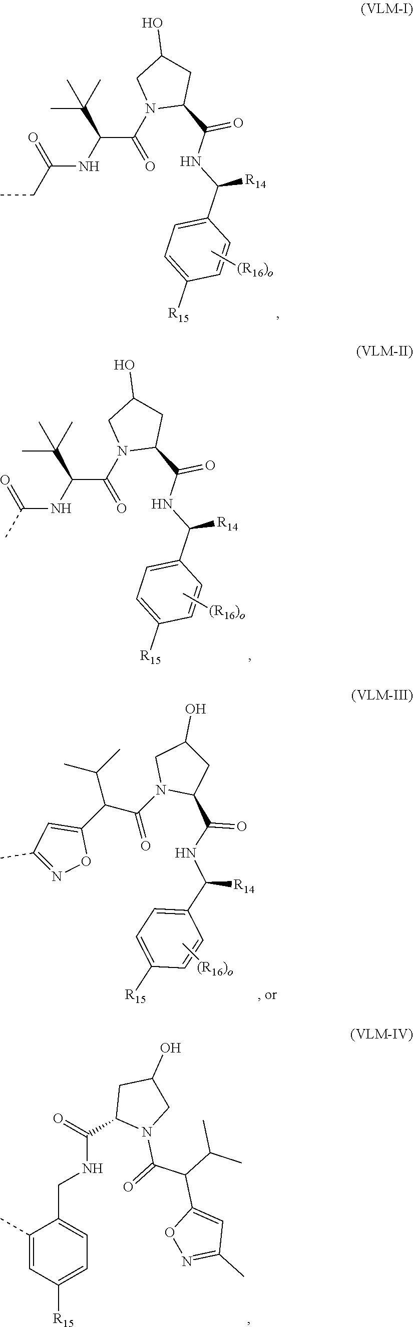

wherein:
   R₁₄ is H or a linear or branched C₁-C₃ alkyl (e.g., methyl);
   R₁₅ is a 5-membered heteroaryl having one or two heteroatoms selected from N, S, and O, optionally substituted with a methyl;
   R₁₆ is a halo, optionally substituted C1-C3 alkyl, optionally substituted C—C3 haloalkyl, hydroxy, optionally substituted C1-C3 alkoxy, or optionally substituted C1-C3 haloalkoxy; and
   is an integer from 0-2 (e.g., 0, 1, or 2); and
(c) the L is a chemical linking moiety that covalently couples the VLM to the PTM.

In any aspect or embodiment described herein, the compound is represented by the chemical structure:

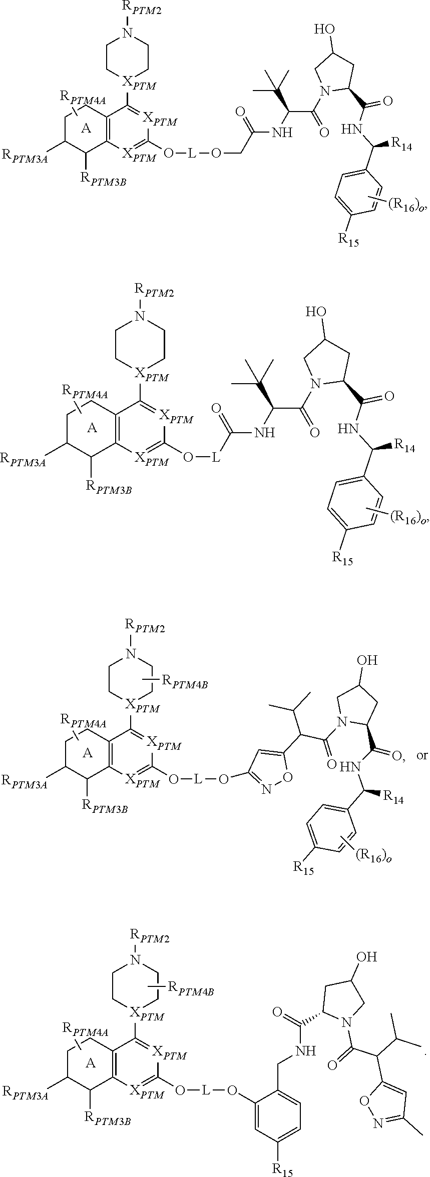

In any aspect or embodiment described herein, the PTM is represented by the chemical structure:

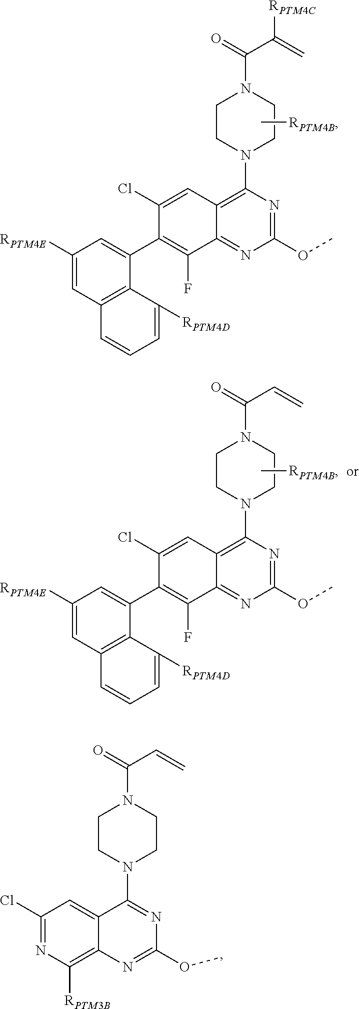

wherein:
R_{PTM4B} is (1) H or absent, (2) —CH₂—CH₂—CN or —CH₂—CN, or (3) 1 or 2 independently selected C₁-C₃ alkyl (e.g., methyl or ethyl);
R_{PTM4D} is a hydrogen, C₁-C₃ alkyl (e.g., methyl), or a halogen (e.g., F, Cl, Br);
R_{PTM4C} is H or halogen (e.g., Cl, F, Br);
R_{PTM4E} is H or OH; and
R_{PTM3B} is —O-indazole, optionally substituted by 1 or 2 groups independently selected from OH and halogen (e.g., F, Cl, Br).

In any aspect or embodiment described herein, the PTM has the chemical structure:

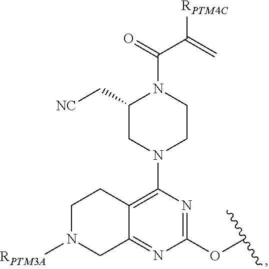

wherein:
R_{PTM3A} is:

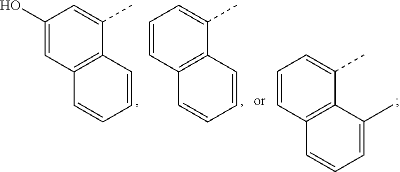

and
R_{PTM4C} is H or F.

In any aspect or embodiment described herein, one of:

(a) the PTM is selected from:

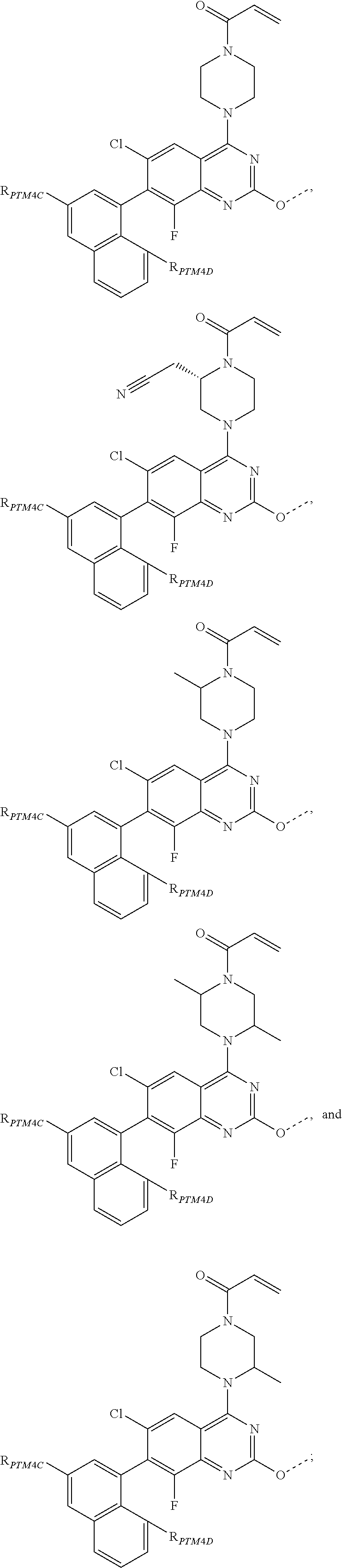

or
(b) the PTM is selected from:

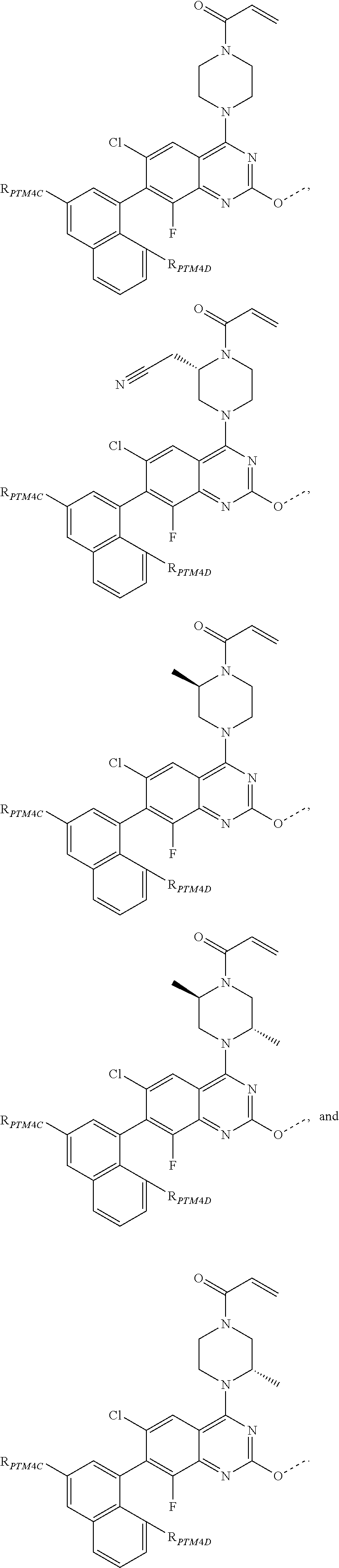

In any aspect or embodiment described herein, the PTM is represented by the chemical structure:

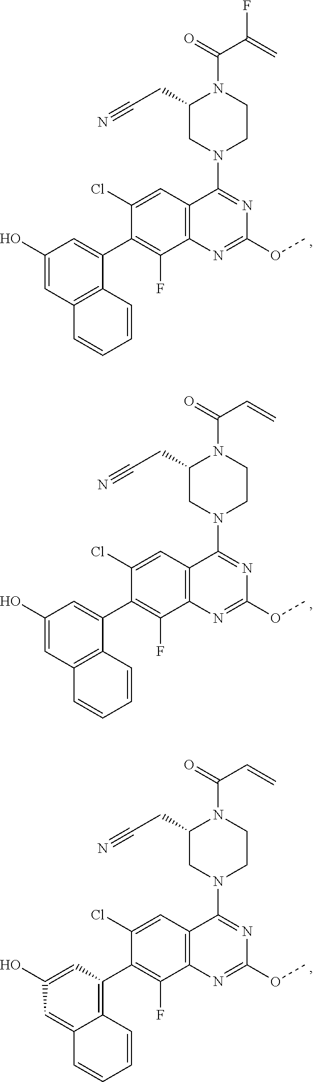

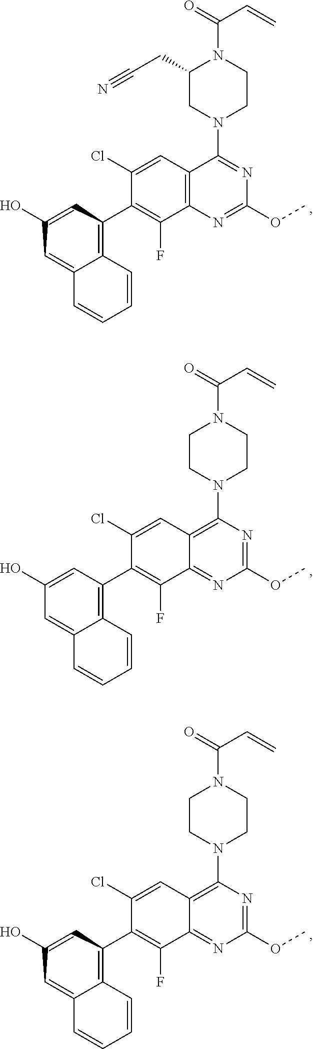

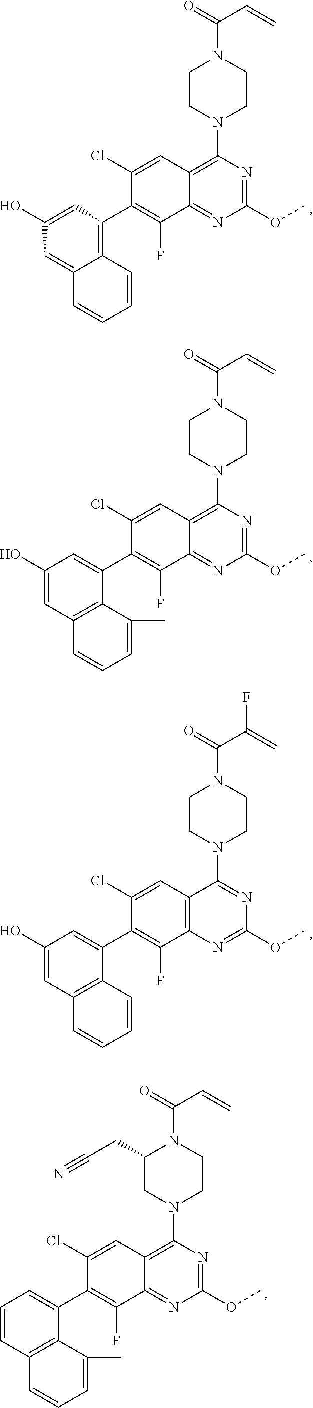

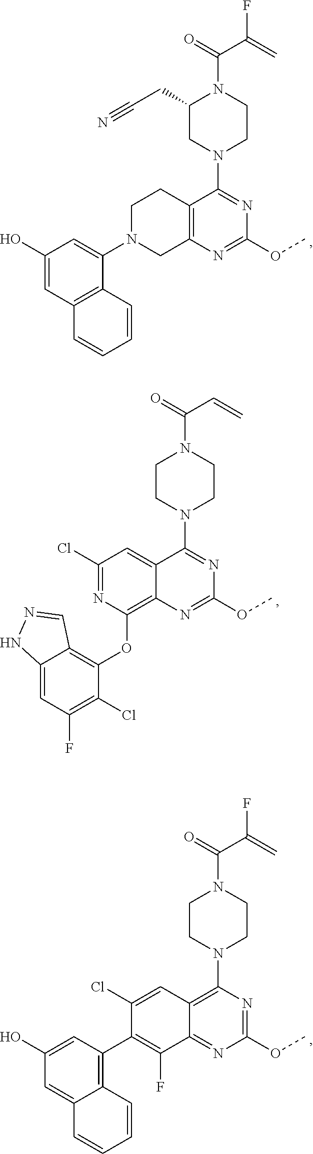

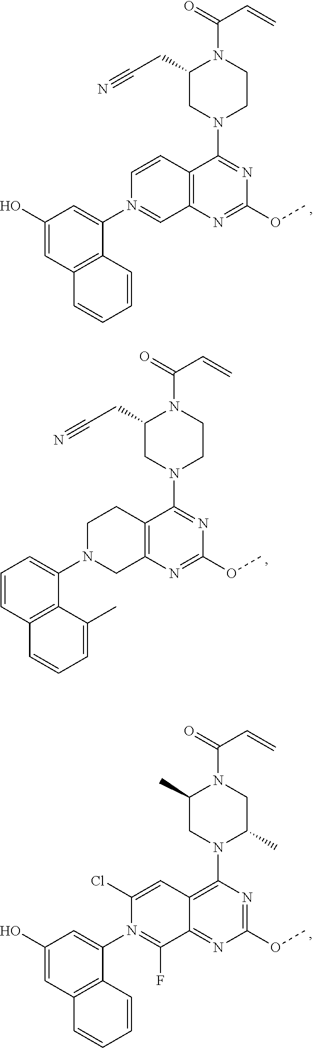

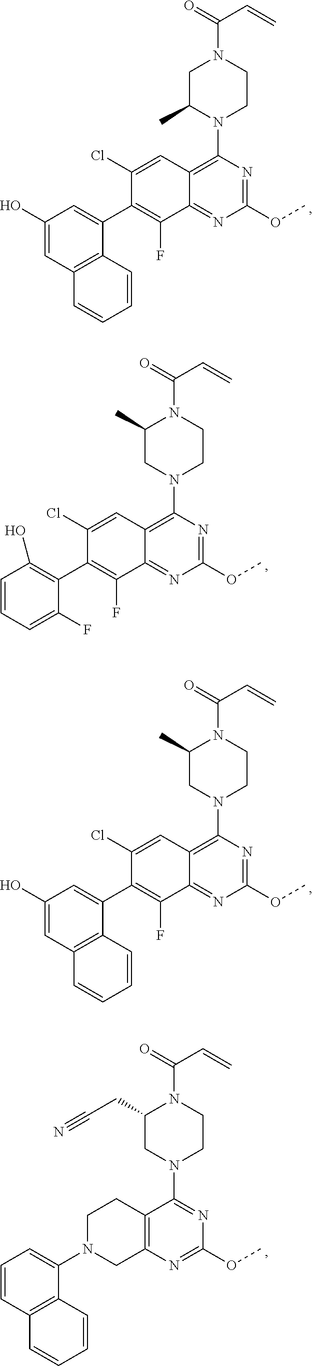

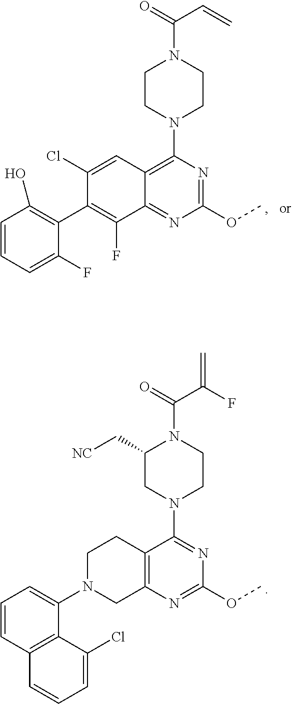

In any aspect or embodiment described herein, the VLM has a chemical structure represented by:

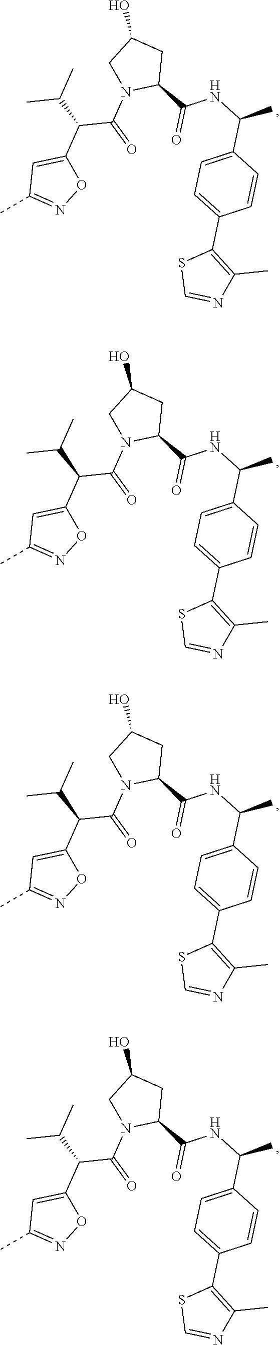

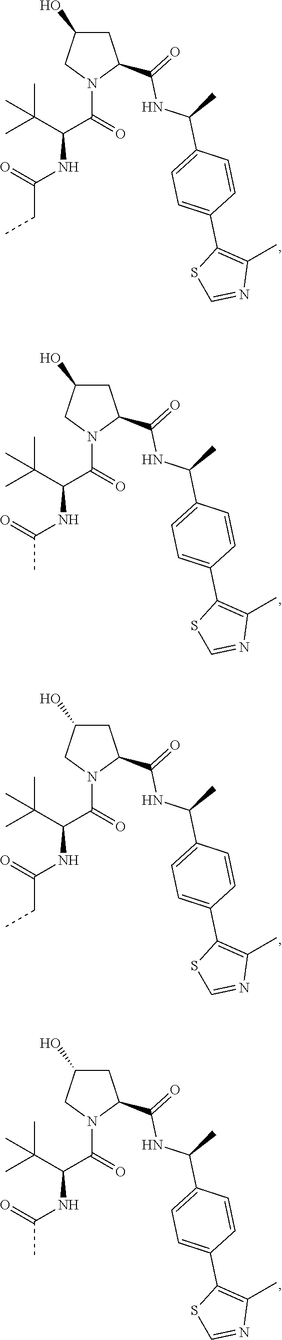

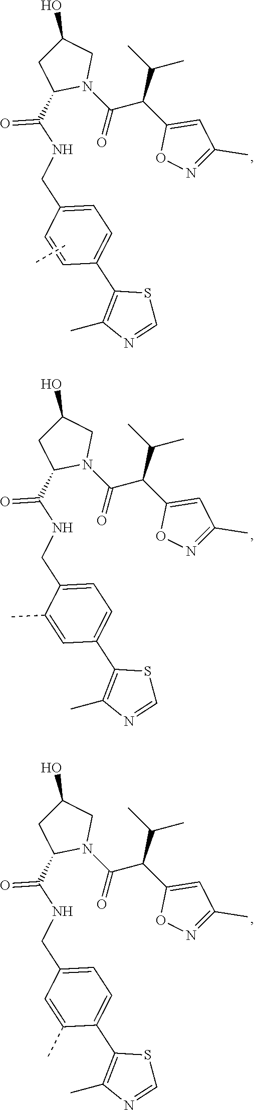

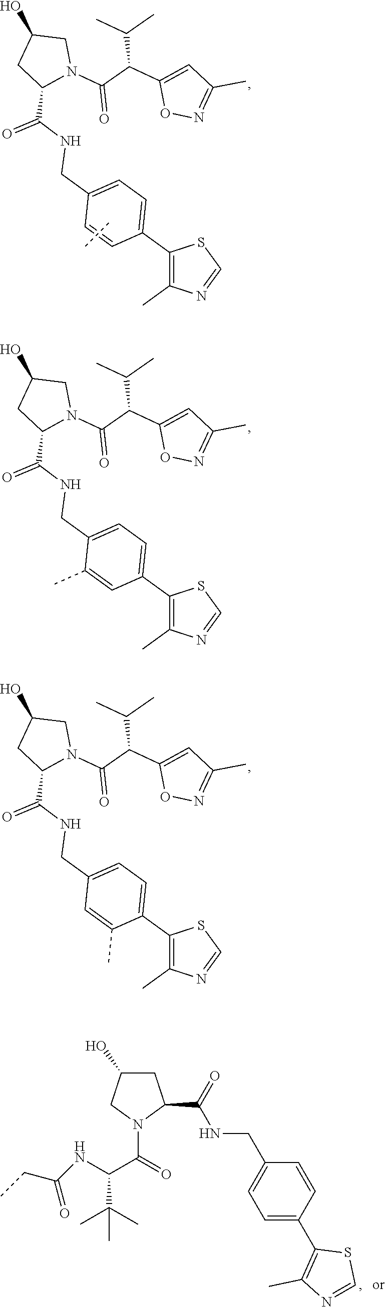

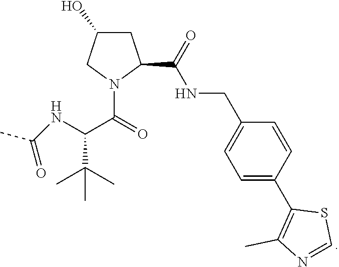

In any aspect or embodiment described herein, the linker (L) comprises the following chemical structure:

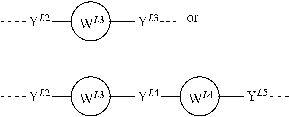

Y^{L2} is a bond, or a unsubstituted or substituted linear or branched C1-C4 alkyl (e.g., optionally substituted with a halogen, C1-3 alkyl, methyl, or ethyl);
W^{L3} is a 3-7 membered ring (e.g., 4-6 membered cycloalkyl or heterocycloalkyl) with 0-4 heteroatoms, optionally substituted with halo or methyl;
Y^{L3} is a bond or a C1-C32 alkyl (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, or C₃₂ alkyl), wherein one or more C atoms are optionally replaced with O and each carbon is optionally substituted with a halogen, a methyl or ethyl;
Y_{L4} is bond, O, or an unsubstituted or substituted linear or branched C1-C6 alkyl, wherein one or more carbons are optionally replaced O, NH, or NCH₃, and optionally substituted with a halo or methyl;
W^{L4} is a 3-8 membered ring (e.g., 4-6 membered cycloalkyl or heterocycloalkyl) or a 5-8 member spirocyclic, each with 0-4 heteroatoms and optionally substituted with halo (e.g., F, Cl, Br), or methyl; and
Y^{L5} is a bond or an unsubstituted or substituted C1-C6 alkyl, where one or more C atoms are optionally replaced with O and optionally substituted with a halo (e.g., F, Cl, Br), or methyl.

In any aspect or embodiment described herein, the linker has a chemical structure represented by:

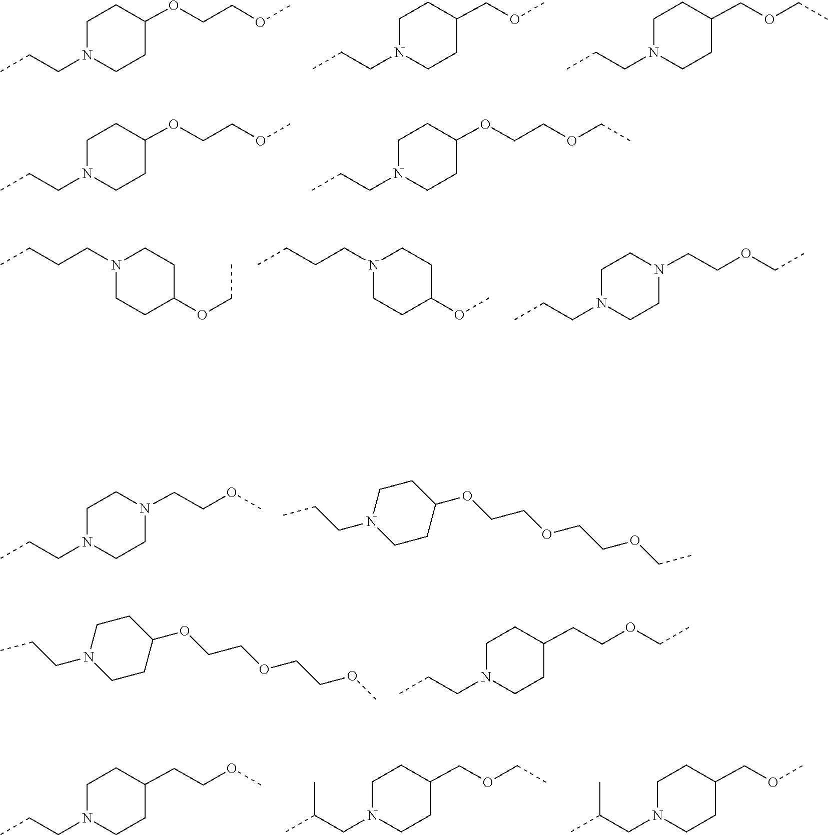

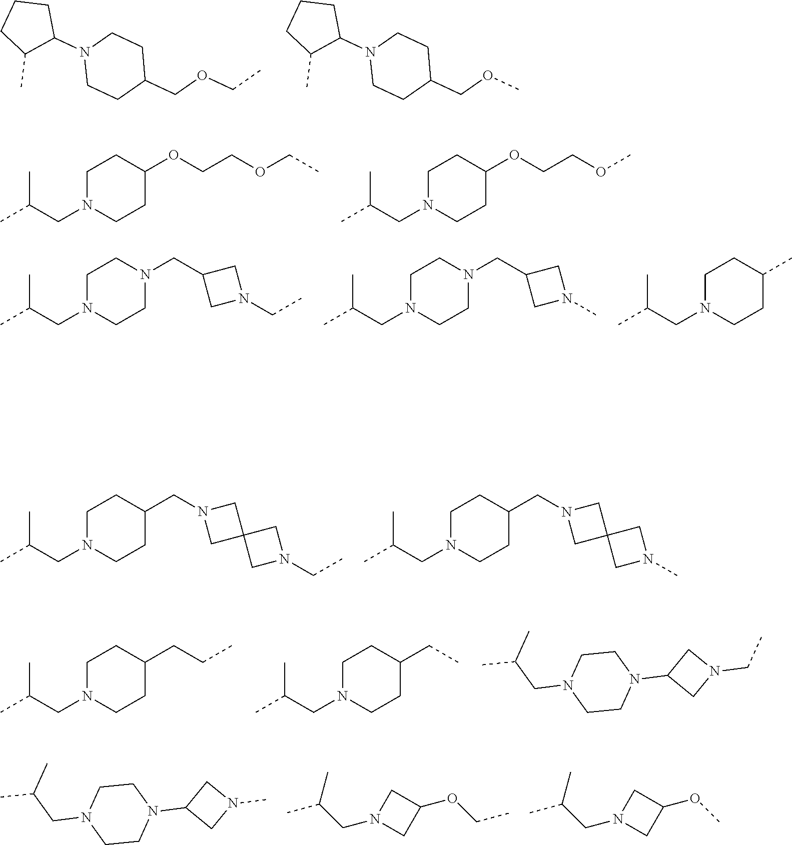

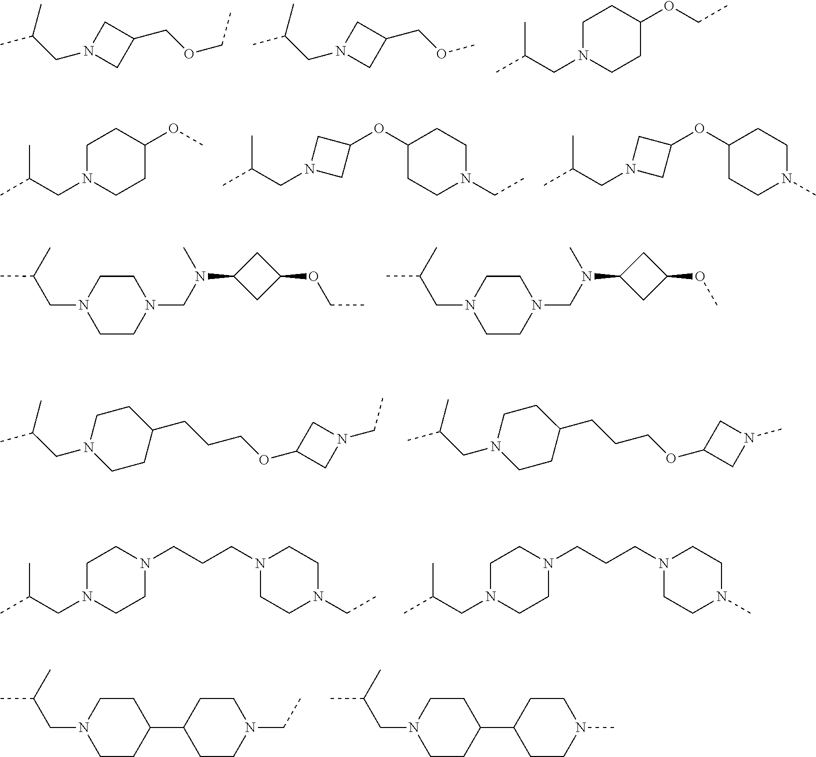

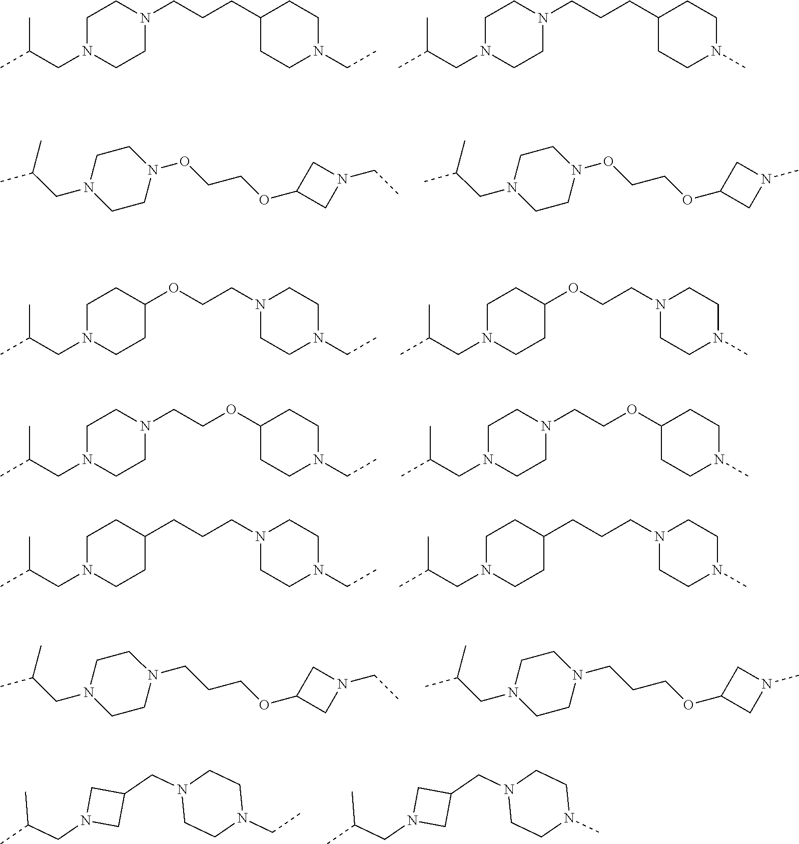

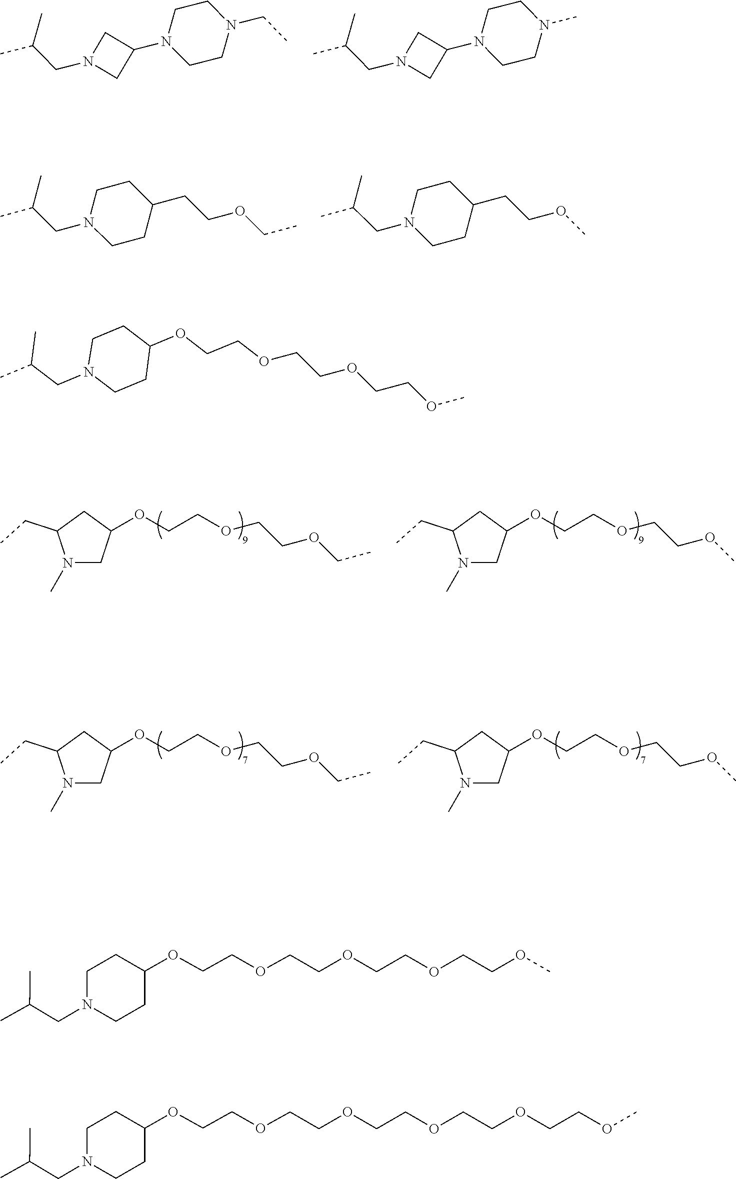

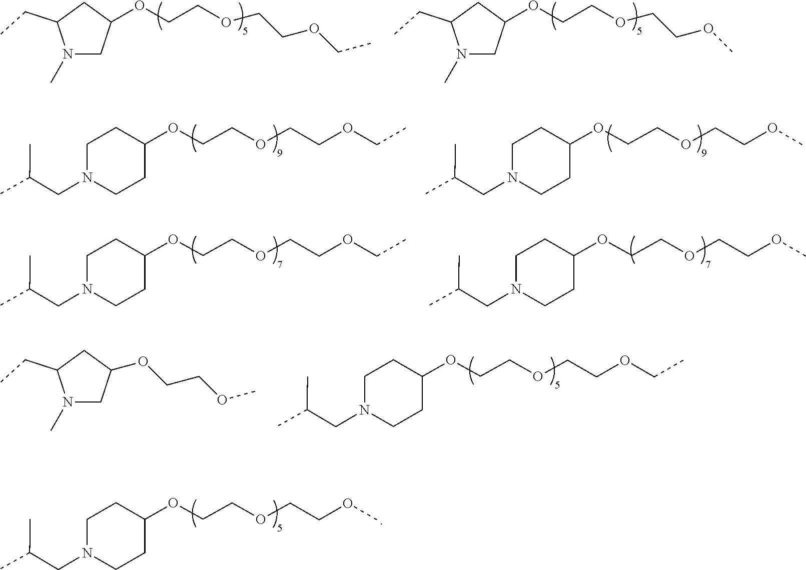

In any aspect or embodiment described herein, the linker (L) is represented by:

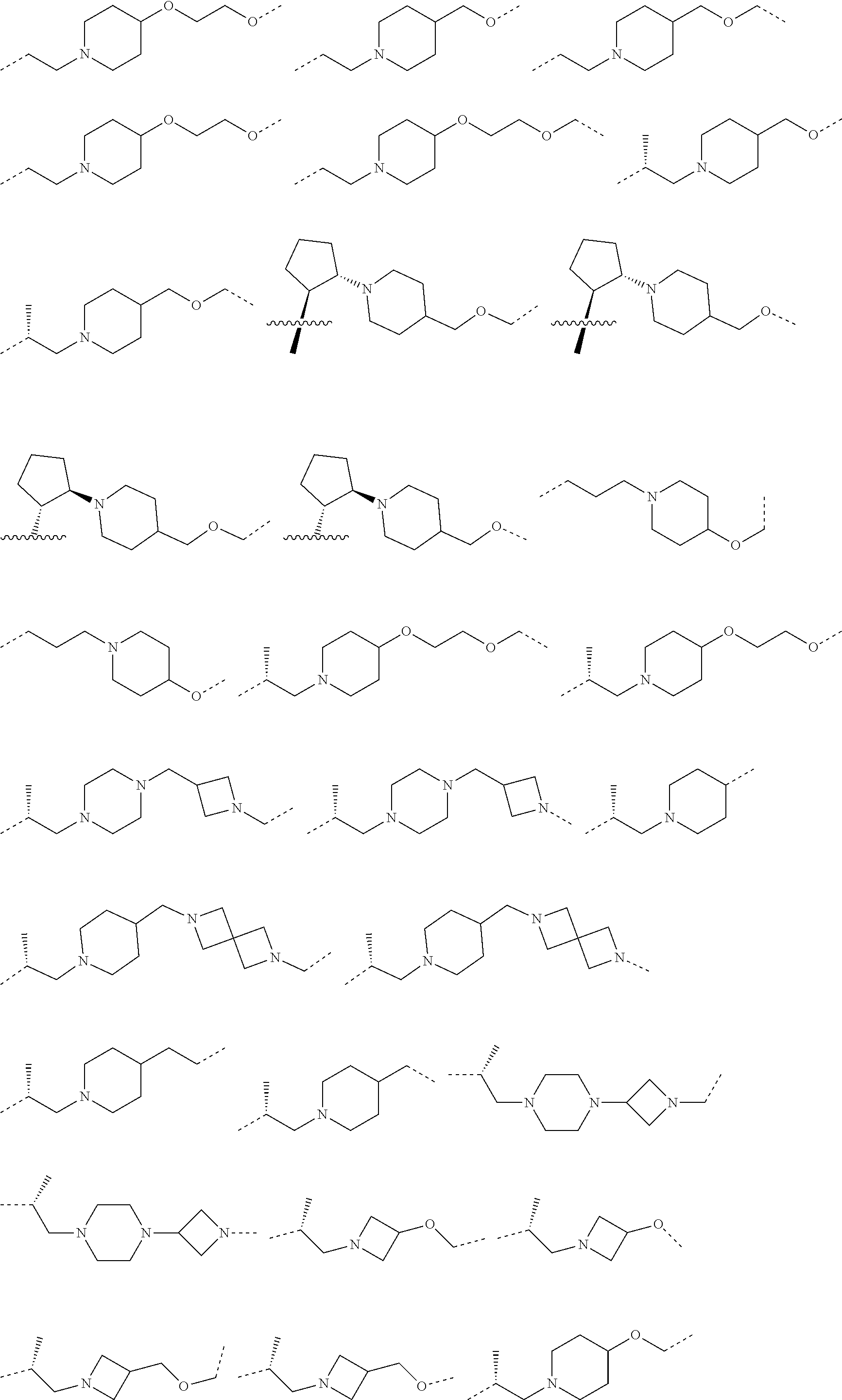

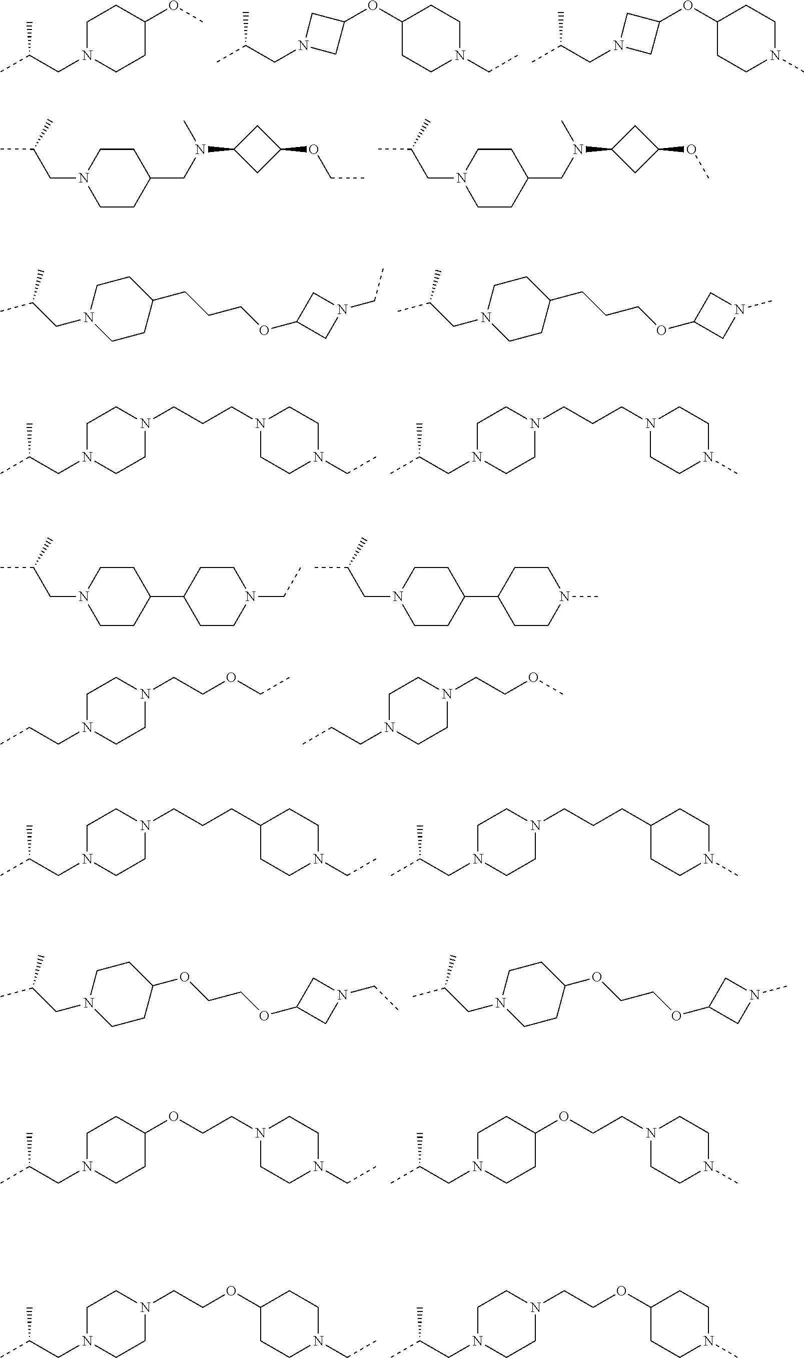

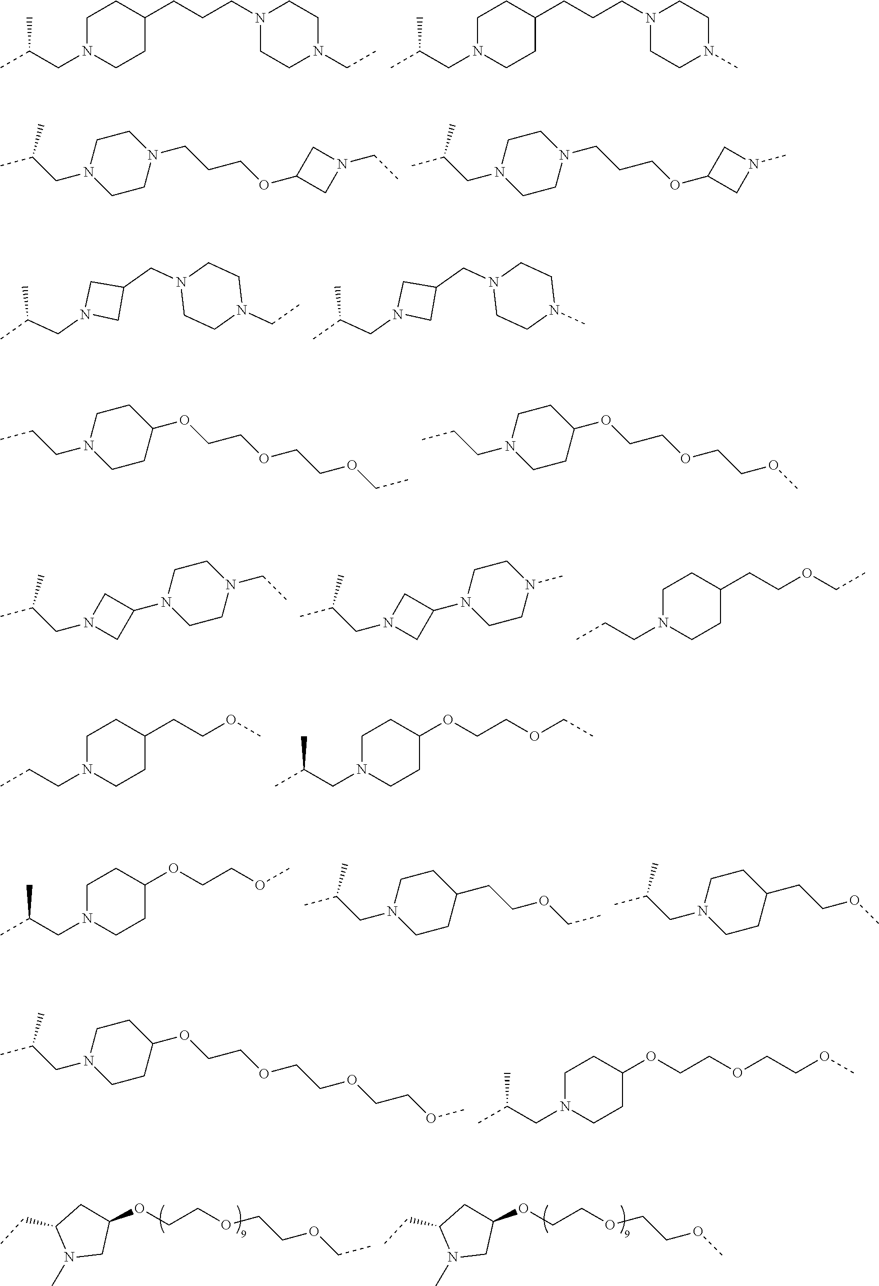

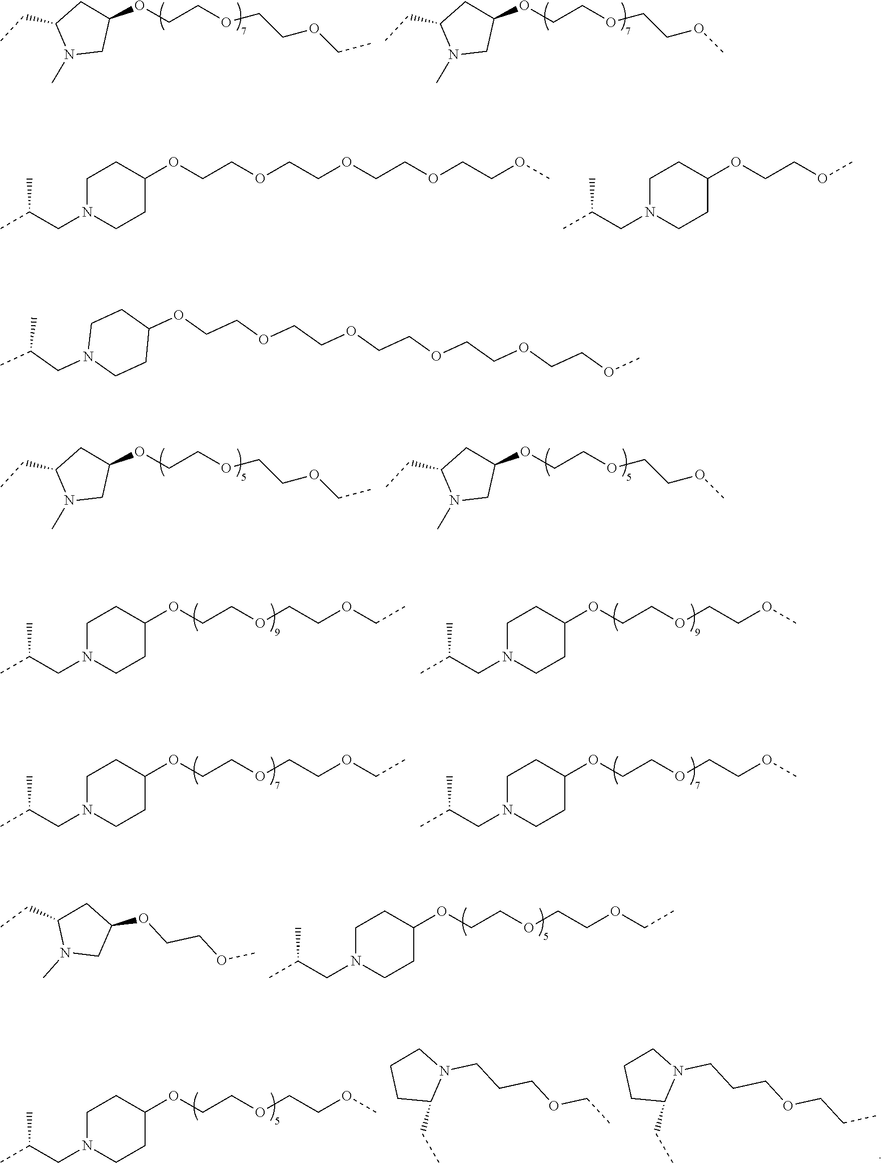

In any aspect or embodiment described herein, at least one of: the PTM is a PTM selected from compounds 1-90 of Table 7; the ULM is a ULM selected from compounds 1-90 of Table 7; and the L is an L selected from compounds 1-90 of Table 7.

In any aspect or embodiment described herein, the compound is selected from the group consisting of compounds 1-90 of Table 7.

In any aspect or embodiment described herein, the composition comprises an effective amount of a bifunctional compound as described herein, and a pharmaceutically acceptable carrier.

In any aspect or embodiment described herein, the composition further comprises at least one of additional bioactive agent or a second bifunctional compound as described herein.

In any aspect or embodiment described herein, the additional bioactive agent is an anti-inflammatory, a chemotherapy agent, or an immunomodulatory agent.

In any aspect or embodiment described herein, the composition comprises a pharmaceutically acceptable carrier and an effective amount of at least one compound as described herein for treating a disease, a disorder or a symptom casually related to KRas in a subject, wherein the composition is effective in treating or ameliorating the disease, disorder, or at least one symptom of the disease or disorder.

In any aspect or embodiment described herein, the disease or disorder is pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

In any aspect or embodiment described herein, the method of treating or preventing a disease, a disorder, or symptom associated with KRas comprises providing a patient in need thereof, and administering an effective amount of a compound as described herein or composition comprising the same to the patient, wherein the compound or composition is effective in treating or ameliorating the disease, disorder, or at least one symptom of the disease or disorder.

In any aspect or embodiment described herein, the hetero-bifunctional compound has the chemical structure:

PTM-L-VLM,

or a pharmaceutically acceptable salt or solvate thereof,

wherein:
(a) the PTM is a small molecule Kirsten ras sarcoma protein (KRas) targeting moiety that binds to KRas^{G12C}, and is represented by the chemical structure:

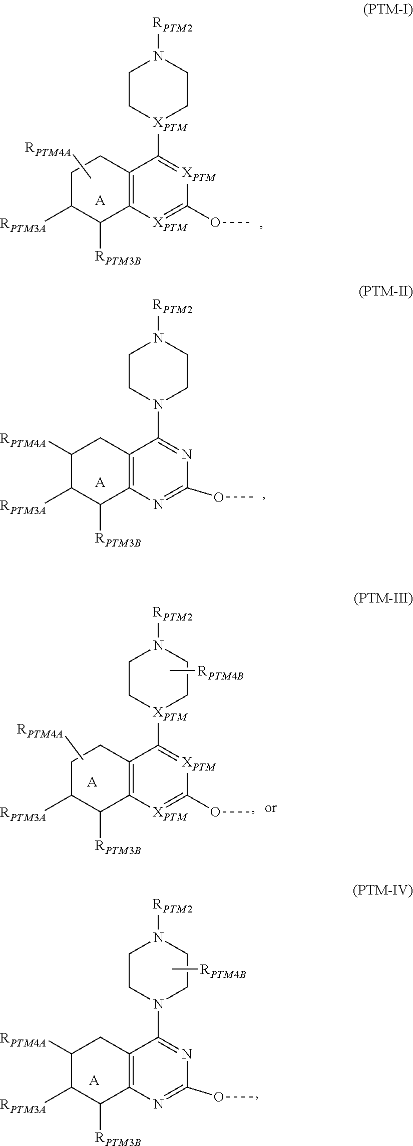

wherein:

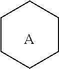

is an 6-membered aryl, 6-membered heteroaryl, or a 6-membered heterocycloalkyl, each optionally substituted with 1 or 2 halogens (e.g., Cl, F, or Br);
R_{PTM2} is —C(═O)C2-C4alkenyl, optionally substituted by a methyl or halogen (e.g., Cl, F, Br);
R_{PTM3A} is H, phenyl, or naphthalene, each optionally substituted by 1, 2, or 3 groups independently selected from OH, halogen (e.g., F, Cl, Br), or a linear or branched C1-C3 alkyl (e.g., methyl or ethyl);
R_{PTM3B} is H, halogen (e.g., Cl, F, Br), or —O—R_{PTM3C}, wherein R_{PTM3C} is an indazole

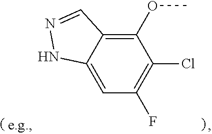

wherein R_{PTM3B} is optionally substituted by 1, 2, or 3 groups independently selected from OH, halogen (e.g., F, Cl, Br), or a linear or branched C1-C3 alkyl (e.g., methyl or ethyl);
R_{PTM4A} of PTM-I or PTM=III is absent (or H), or 1 or 2 independently selected halogen (e.g., Cl, F, Br);
R_{PTM4A} of PTM-II and PTM-IV is absent (or H) or a halogen (e.g., Cl, F, Br);
R_{PTM4B} is (1) absent (or H), (2) —CH₂—CH₂—CN or —CH₂—CN, or (3) 1 or 2 independently selected C₁-C₃ alkyl (e.g., methyl or ethyl); and
each X_{PTM} is individually a CH or N;
(b) the VLM is a small molecule E3 ubiquitin ligase binding moiety that binds a Von Hippel-Lindau (VHL) E3 ubiquitin ligase, and is represented by the chemical structure:

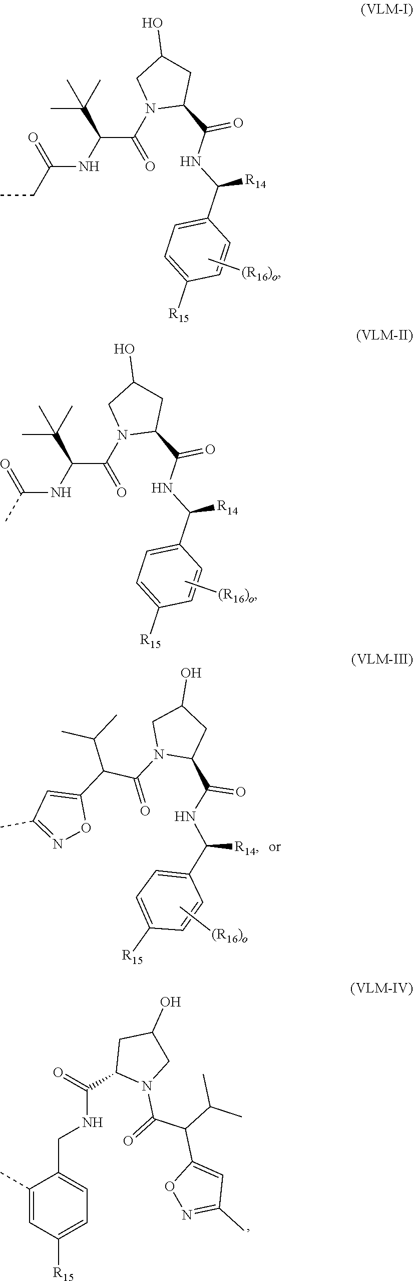

wherein:
   R₁₄ is H or a linear or branched C1-C3 alkyl (e.g., methyl);
   R₁₅ is a 5-membered heteroaryl having one or two heteroatoms selected from N, S, and O, optionally substituted with a methyl;
   R₁₆ a halo, optionally substituted C1-C3 alkyl, optionally substituted C—C3 haloalkyl, hydroxy, optionally substituted C1-C3 alkoxy, or optionally substituted C1-C3 haloalkoxy; and
   is an integer from 0-2 (e.g., 0, 1, or 2); and
(c) the L is a chemical linking moiety that covalently couples the VLM to the PTM.

In any aspect or embodiment described herein, the compound is represented by the chemical structure:

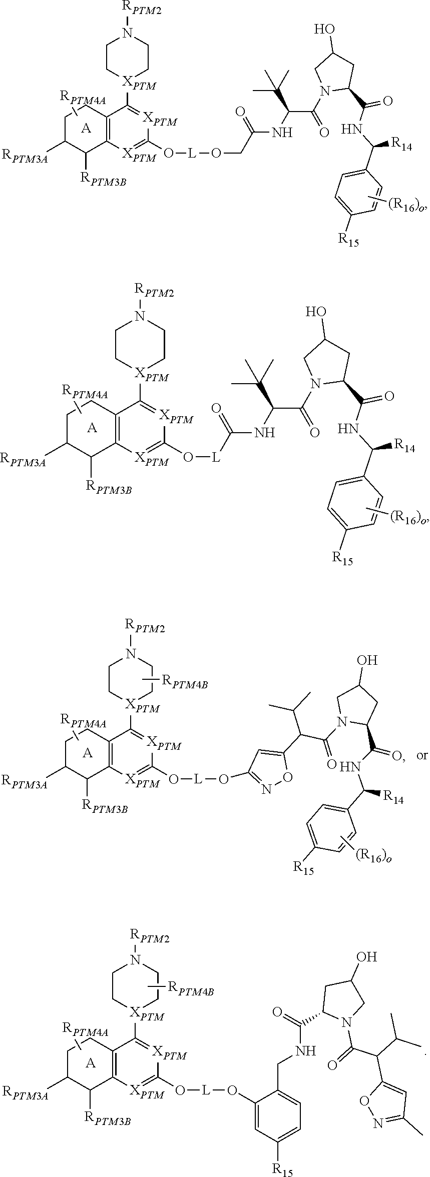

In any aspect or embodiment described herein, the PTM is represented by the chemical structure:

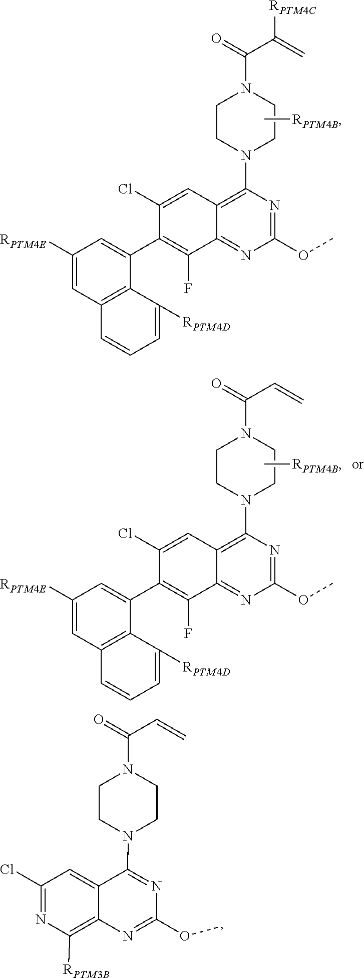

wherein:
R_{PTM4B} is (1) H or absent, (2) —CH₂—CH₂—CN or —CH₂—CN, or (3) 1 or 2 independently selected C1-C3 alkyl (e.g., methyl or ethyl);
R_{PTM4D} is a hydrogen, C1-C3 alkyl (e.g., methyl), or a halogen (e.g., F, Cl, Br);
R_{PTM4C} is H or halogen (e.g., Cl, F, Br);
R_{PTM4E} is H or OH; and
R_{PTM3B} is —O-indazole, optionally substituted by 1 or 2 groups independently selected from OH and halogen (e.g., F, Cl, Br).

In any aspect or embodiment described herein, the PTM has the chemical structure:

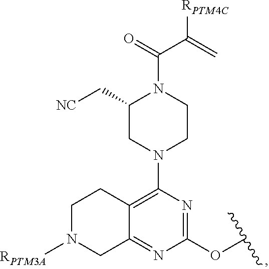

wherein:
R_{PTM3A} is:

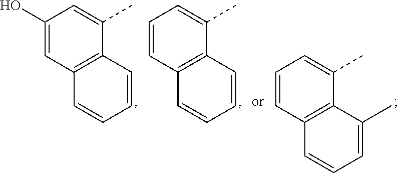

and
R_{PTM4C} is H or F.

In any aspect or embodiment described herein, one of:

(a) the PTM is selected from:

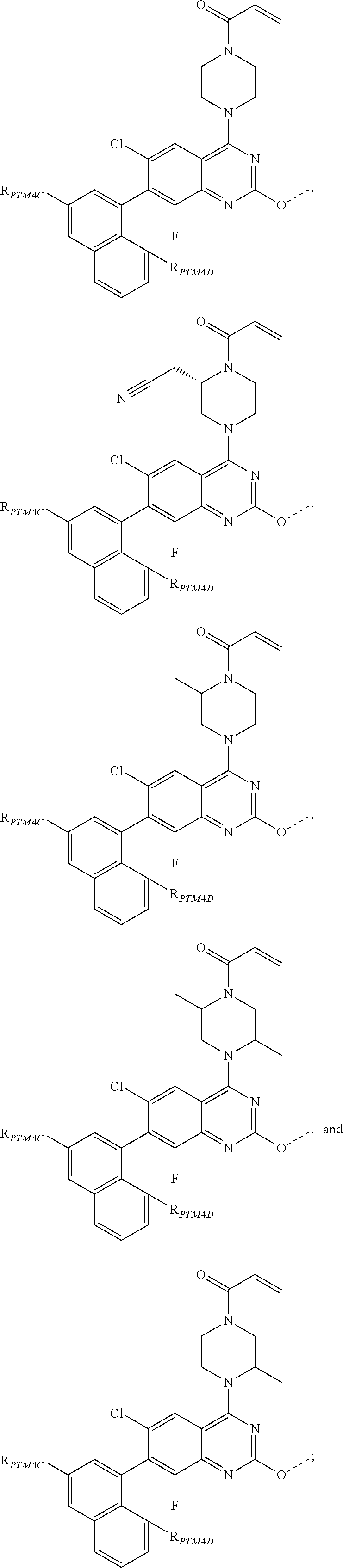

or
(b) the PTM is selected from:

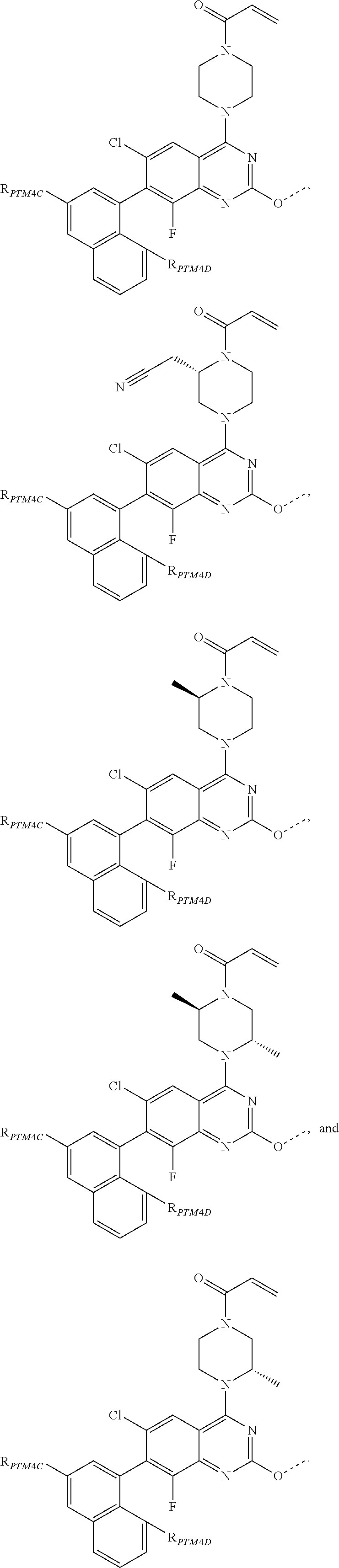

In any aspect or embodiment described herein, the PTM is represented by the chemical structure:

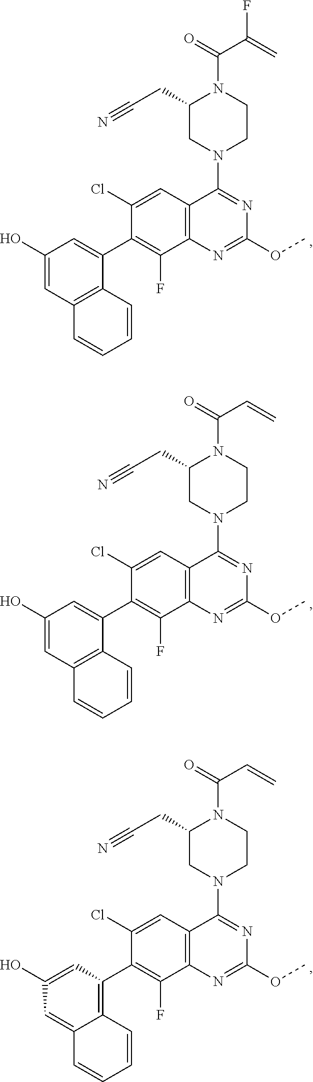

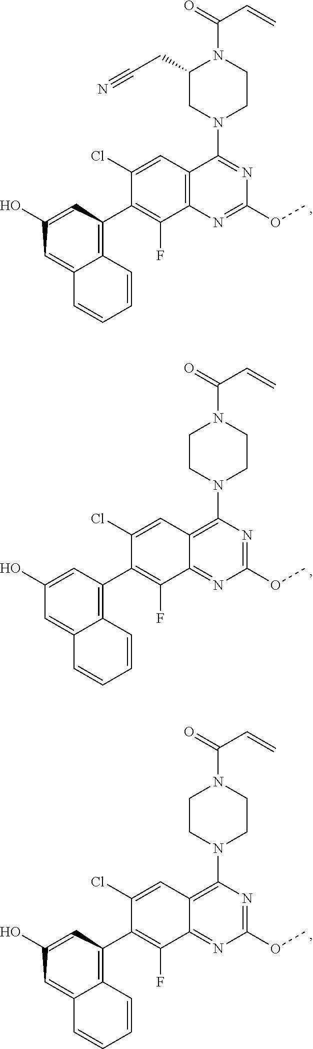

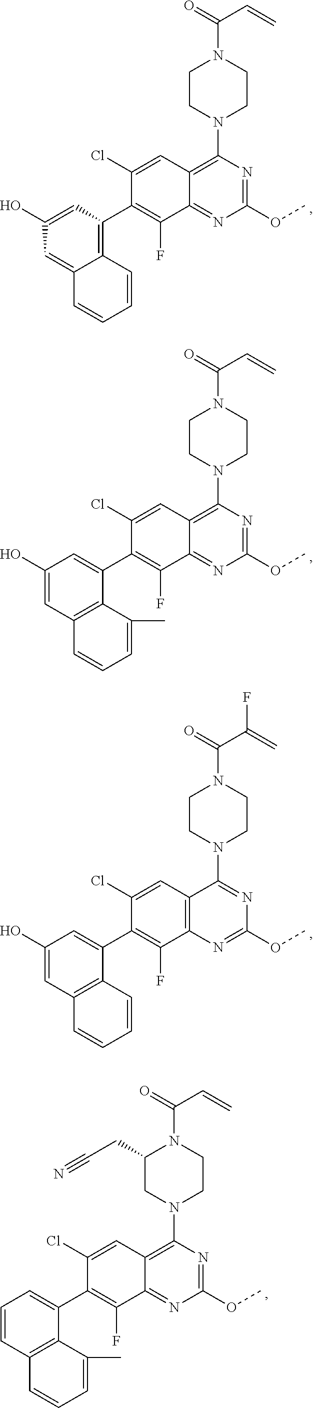

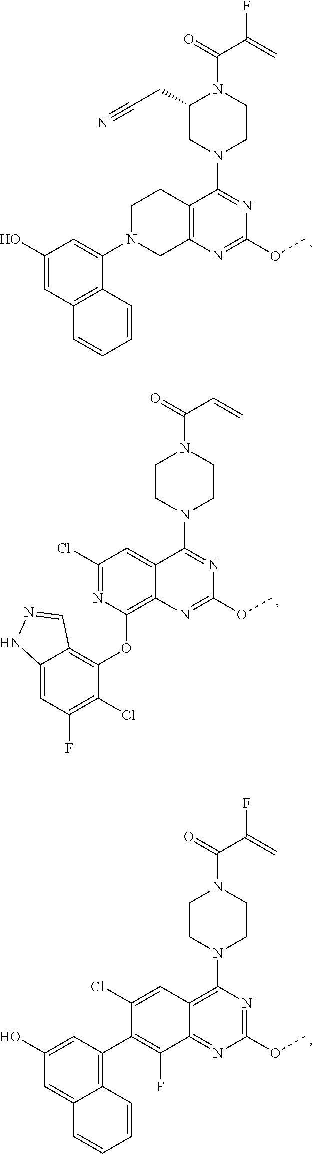

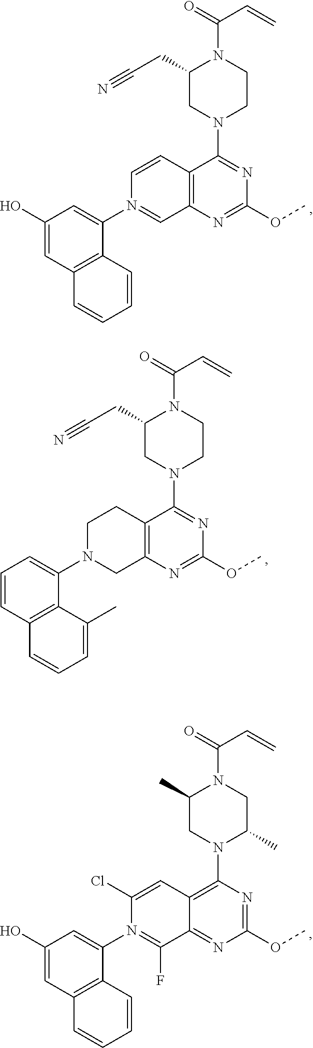

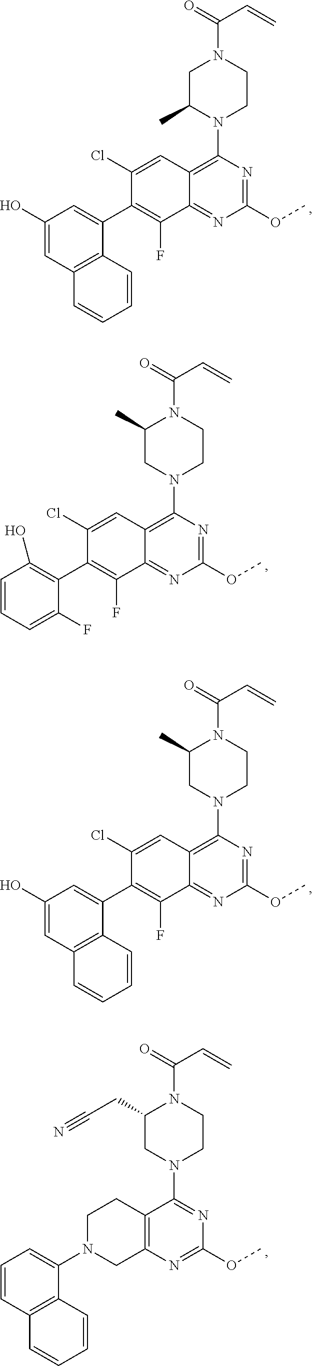

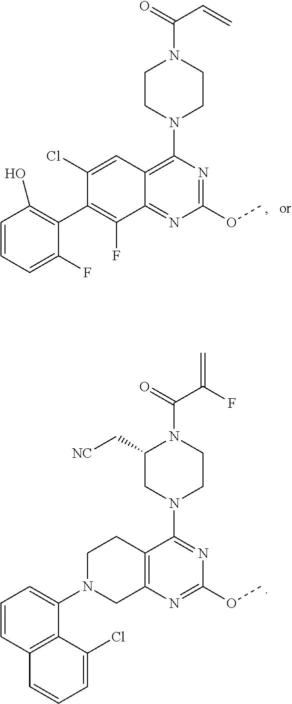

In any aspect or embodiment described herein, the VLM has a chemical structure represented by:

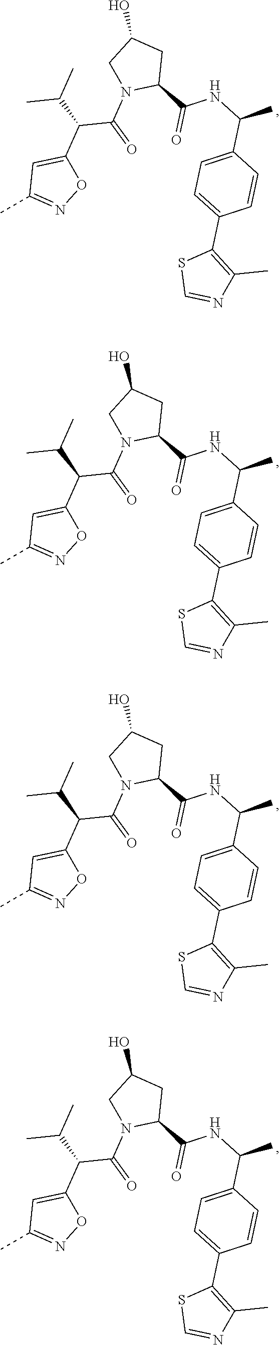

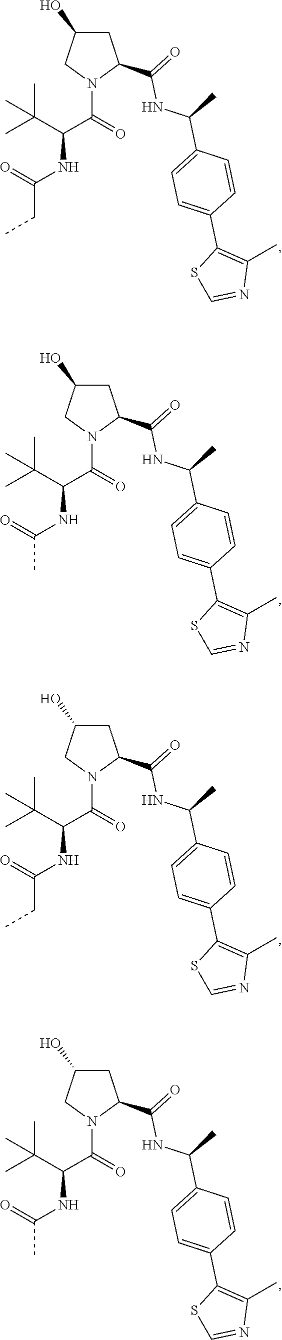

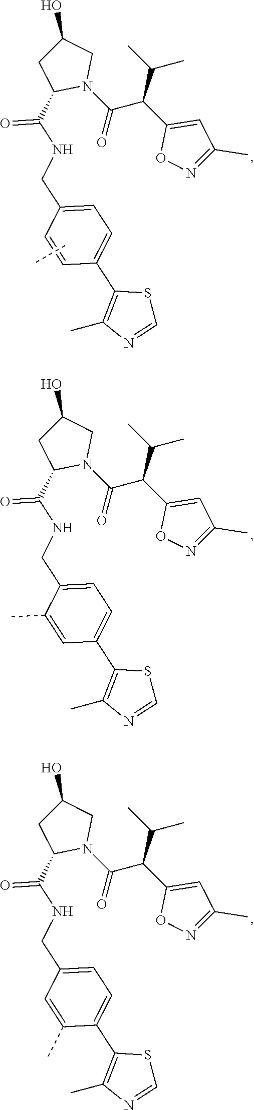

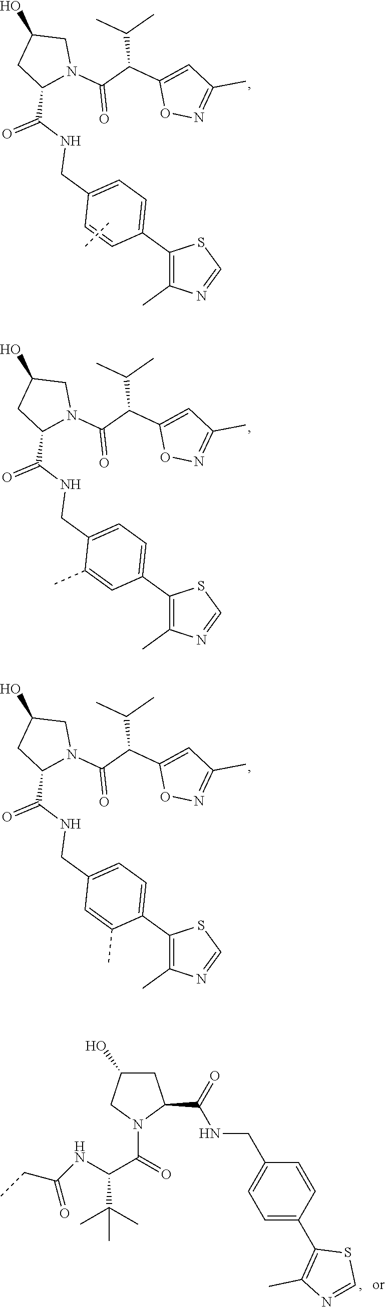

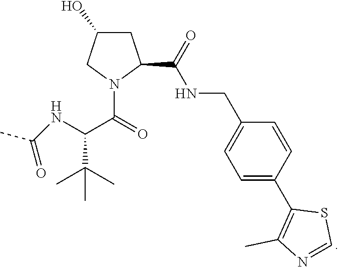

In any aspect or embodiment described herein, the linker (L) comprises the following chemical structure:

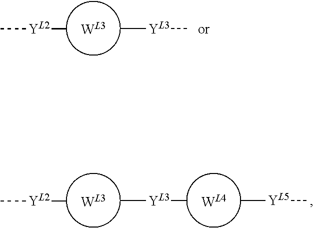

wherein:
Y^{L2} is a bond, or a unsubstituted or substituted linear or branched C1-C4 alkyl (e.g., optionally substituted with a halogen, C1-3 alkyl, methyl, or ethyl);
W^{L3} is a 3-7 membered ring (e.g., 4-6 membered cycloalkyl or heterocycloalkyl) with 0-4 heteroatoms, optionally substituted with halo or methyl;
Y^{L3} is a bond or a C1-C32 alkyl (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, or C₃₂ alkyl), wherein one or more C atoms are optionally replaced with O and each carbon is optionally substituted with a halogen, ═O, a methyl or ethyl;
Y_{L4} is bond, O, or an unsubstituted or substituted linear or branched C1-C6 alkyl, wherein one or more carbons are optionally replaced O, NH, or NCH₃, and optionally substituted with a halo or methyl;
W^{L4} is a 3-8 membered ring (e.g., 4-6 membered cycloalkyl or heterocycloalkyl) or a 5-8 member spirocyclic, each with 0-4 heteroatoms and optionally substituted with halo (e.g., F, Cl, Br), or methyl; and
Y^{L5} is a bond or an unsubstituted or substituted C1-C6 alkyl, where one or more C atoms are optionally replaced with O and optionally substituted with a halo (e.g., F, Cl, Br), or methyl.

In any aspect or embodiment described herein, the linker has a chemical structure represented by:

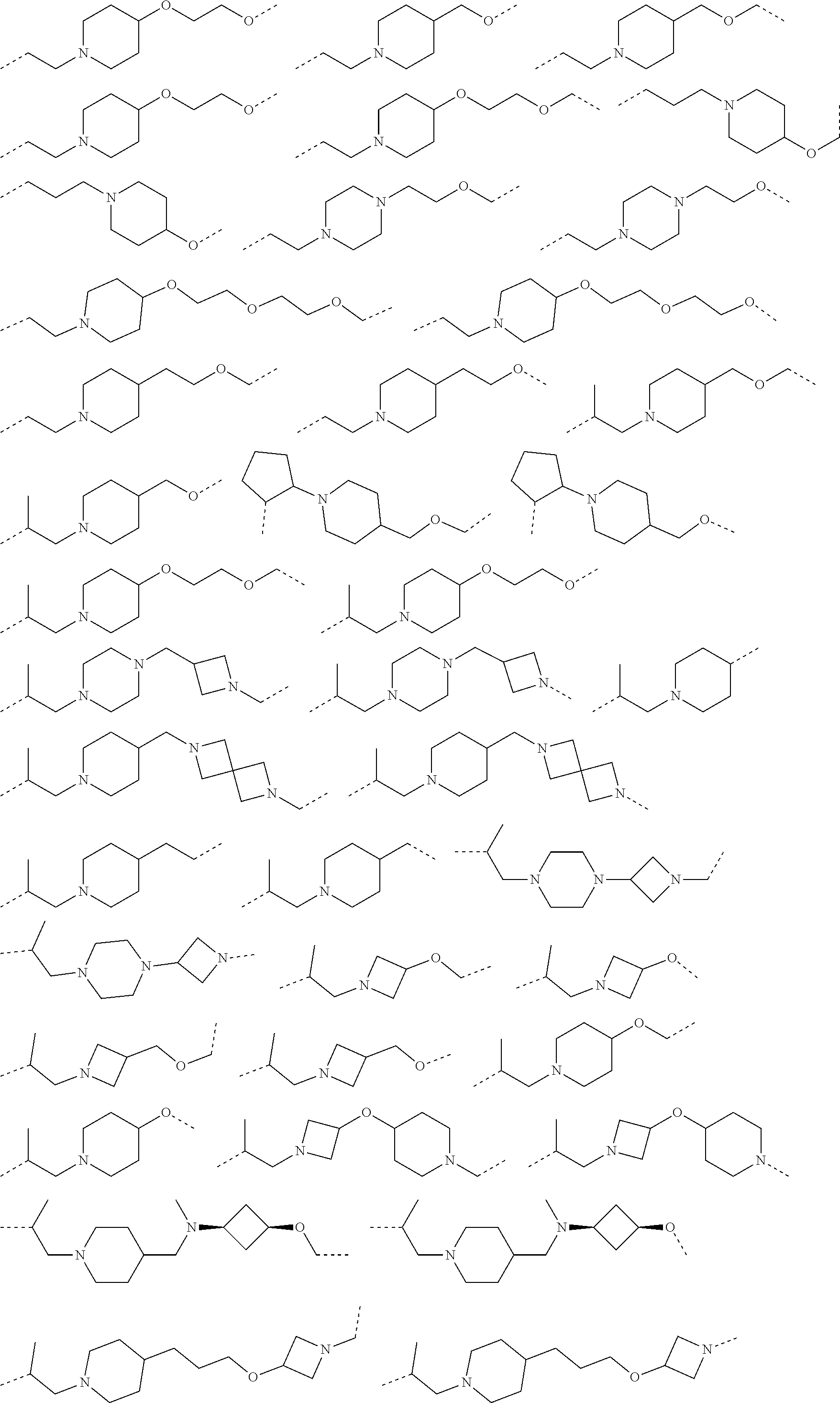

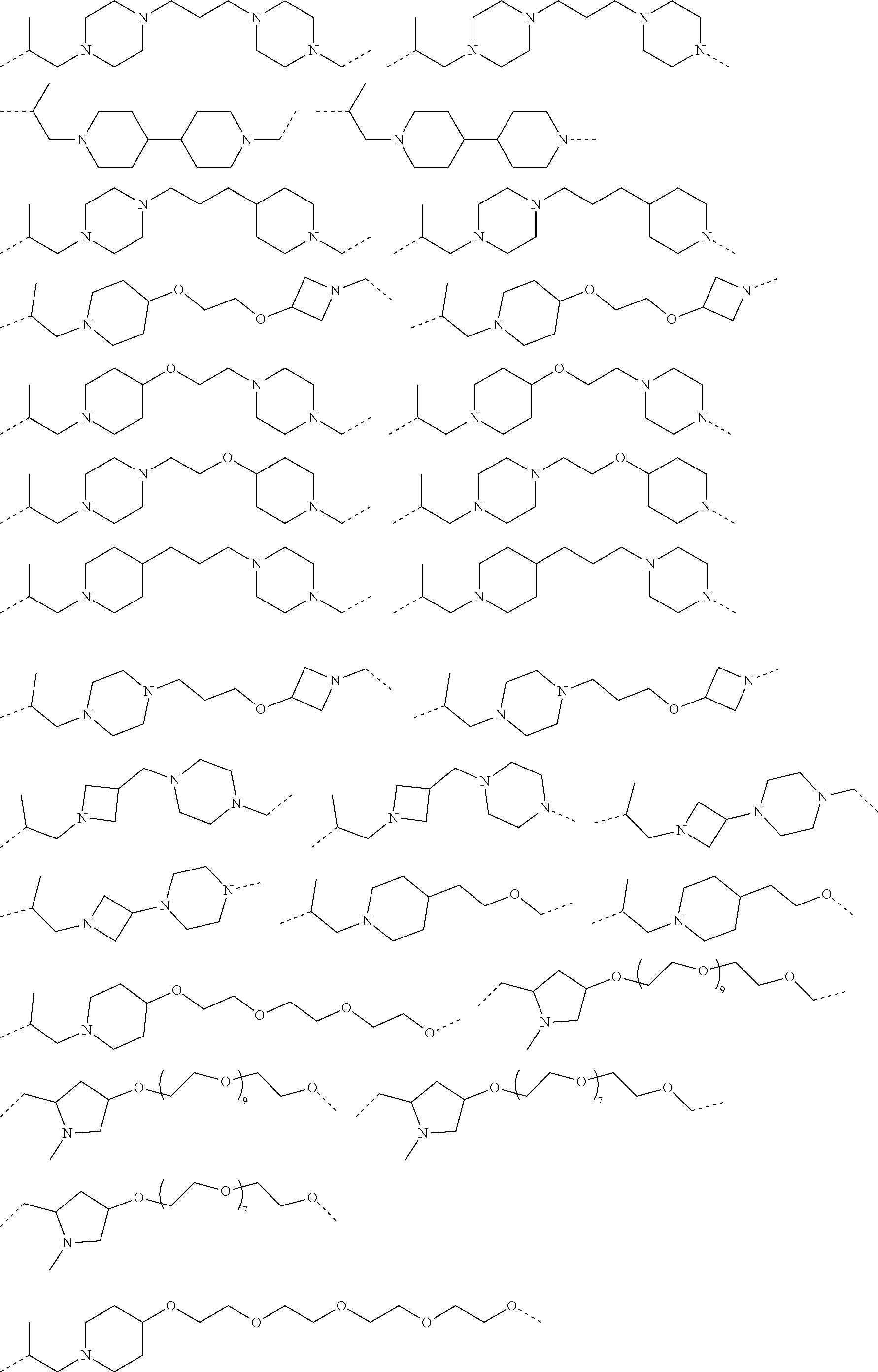

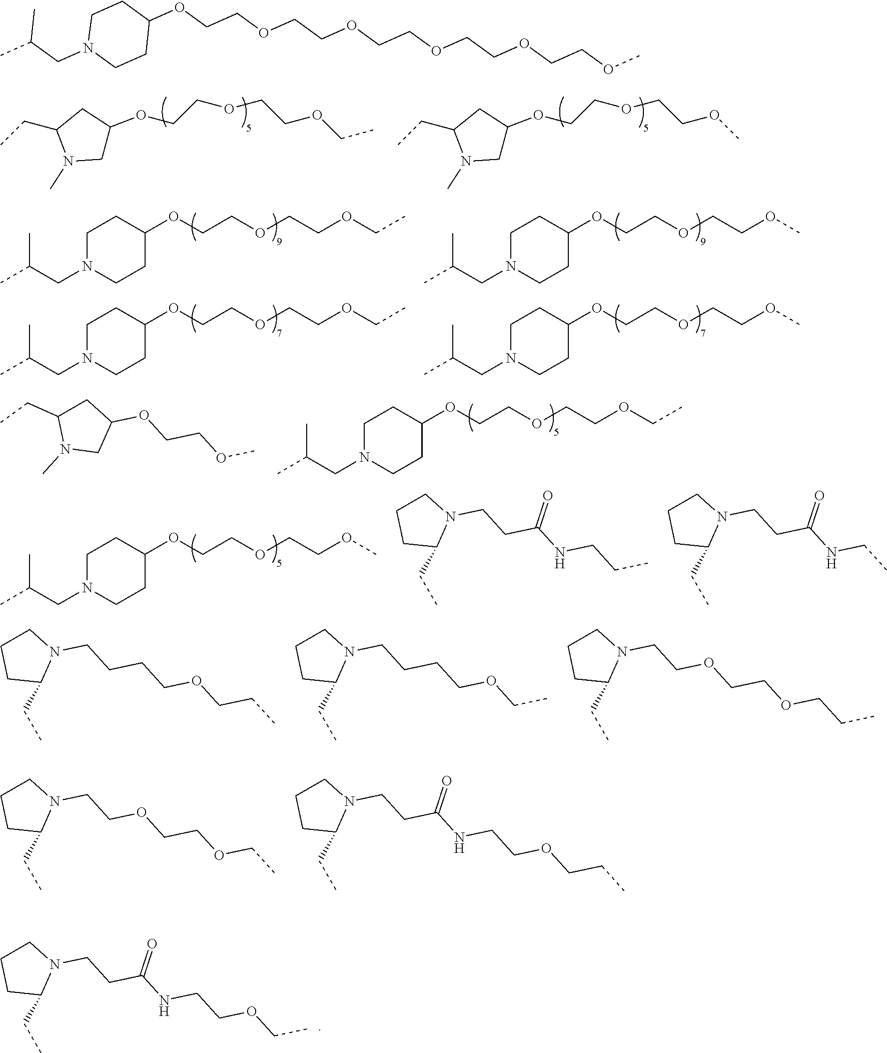

In any aspect or embodiment described herein, the linker (L) is represented by:

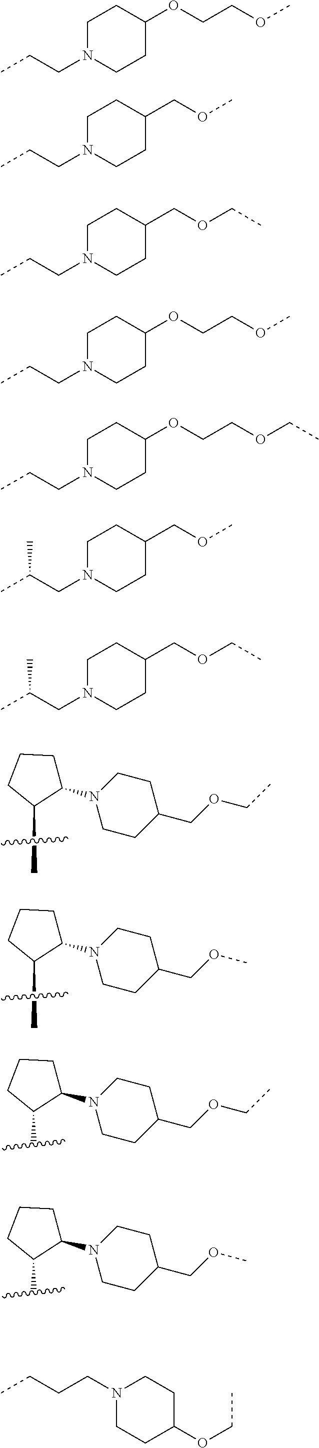

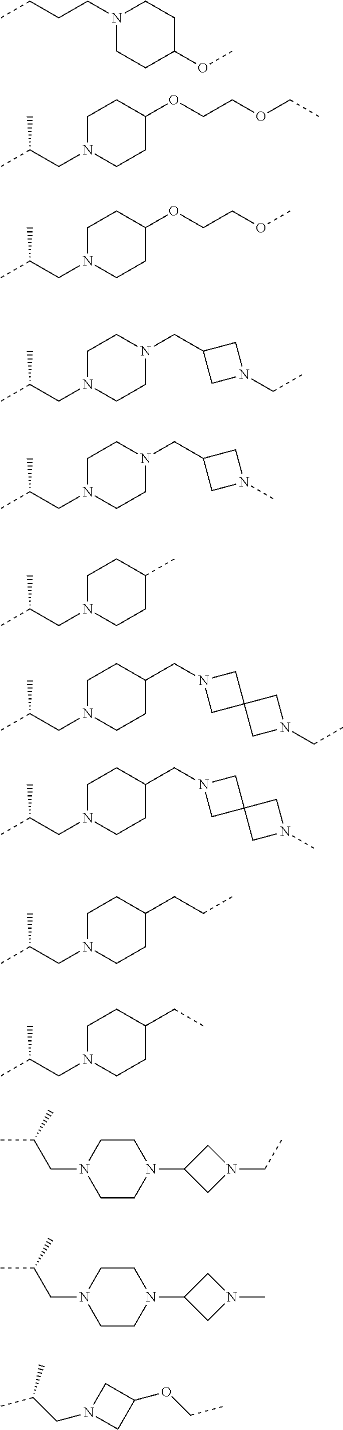

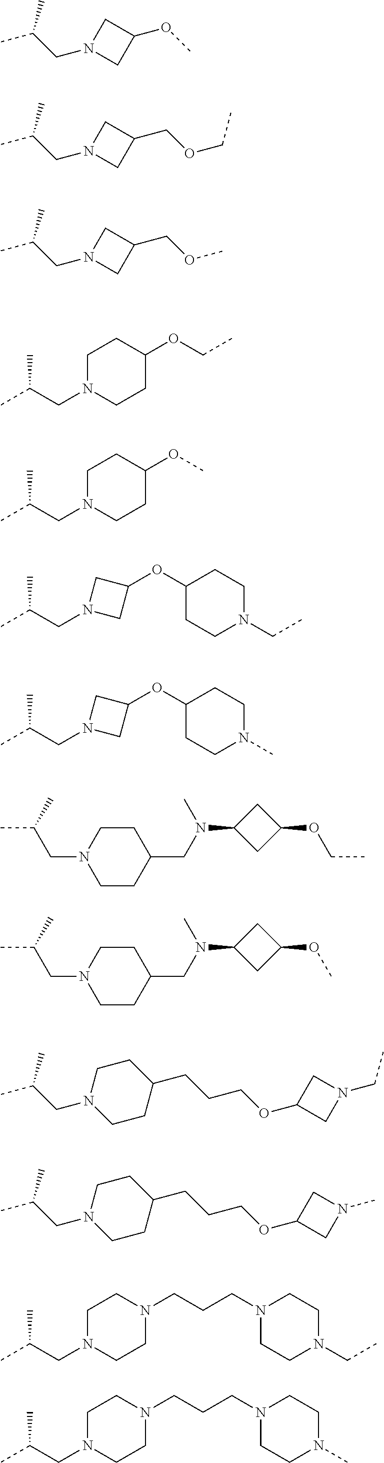

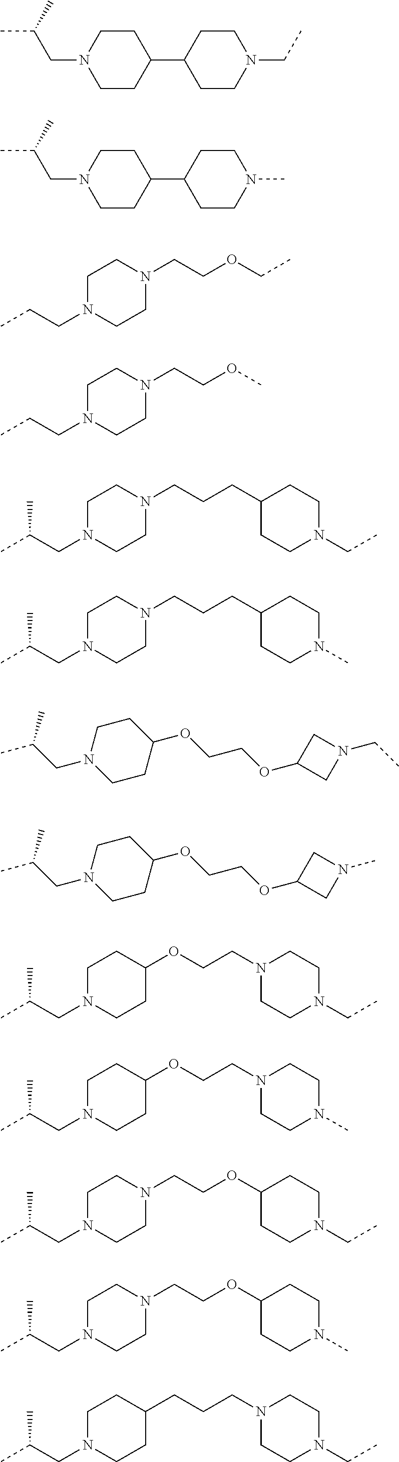

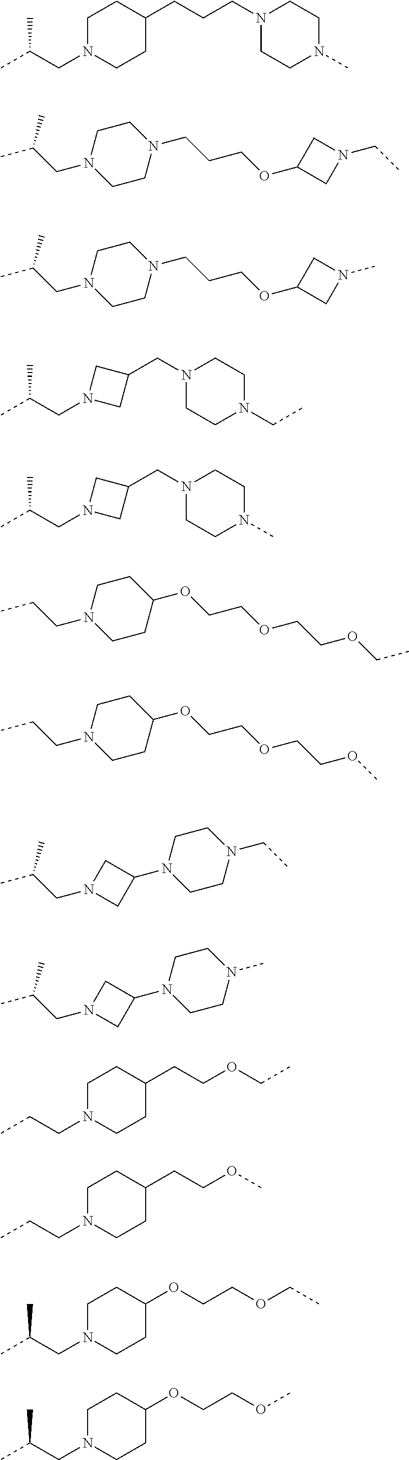

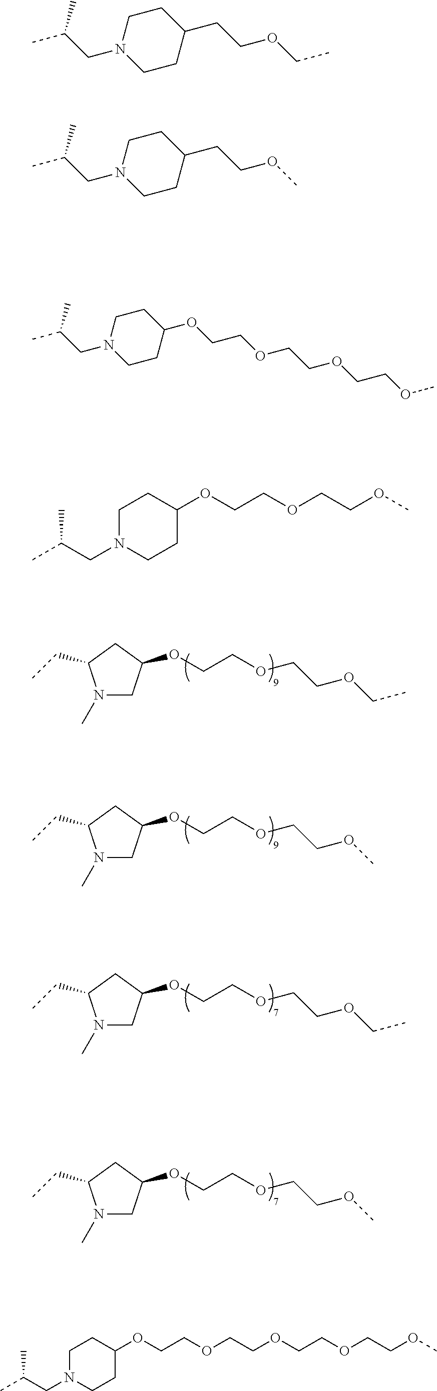

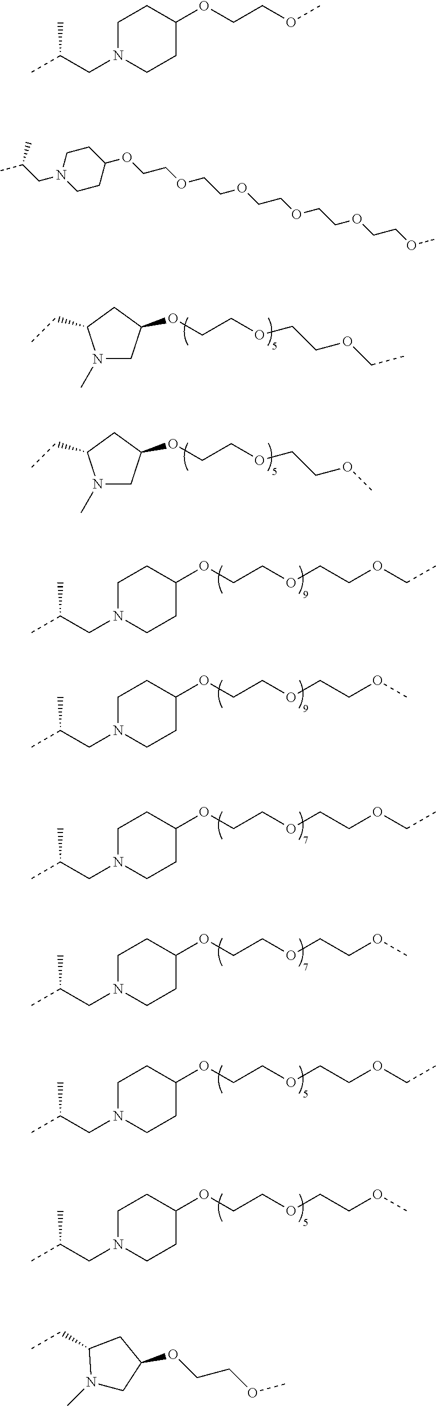

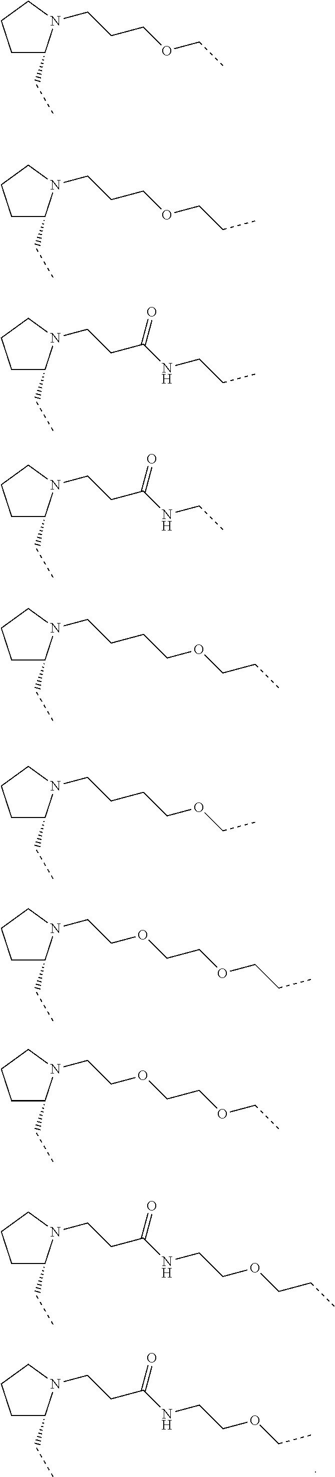

In any aspect or embodiment described herein, at least one of: the PTM is a PTM selected from compounds 1-94 of Table 7; the ULM is a ULM selected from compounds 1-94 of Table 7; and the L is an L selected from compounds 1-94 of Table 7.

In any aspect or embodiment described herein, the compound is selected from the group consisting of compounds 1-94 of Table 7.

In any aspect or embodiment described herein, the composition comprises an effective amount of a bifunctional compound of the present disclosure, and a pharmaceutically acceptable carrier.

In any aspect or embodiment described herein, the composition further comprises at least one of additional bioactive agent or a second bifunctional compound of the present disclosure.

In any aspect or embodiment described herein, the additional bioactive agent is an anti-inflammatory, a chemotherapy agent, or an immunomodulatory agent.

In any aspect or embodiment described herein, the composition comprises a pharmaceutically acceptable carrier and an effective amount of at least one compound of the present disclosure for treating a disease, a disorder or a symptom casually related to KRas in a subject, wherein the composition is effective in treating or ameliorating the disease, disorder, or at least one symptom of the disease or disorder.

In any aspect or embodiment described herein, the disease or disorder is pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

In any aspect or embodiment described herein, the method of treating or preventing a disease, a disorder, or symptom associated with KRas comprises providing a patient in need thereof, and administering an effective amount of a compound as described herein or composition comprising the same to the patient, wherein the compound or composition is effective in treating or ameliorating the disease, disorder, or at least one symptom of the disease or disorder.

A novel bifunctional molecule, which contains a KRas recruiting moiety and an E3 ubiquitin ligase recruiting moiety is described. The bifunctional molecules of the present disclosure actively degrades KRas, leading to robust cellular proliferation suppression and apoptosis induction. Protein degradation mediated by the bifunctional compounds of the present disclosure provides a promising strategy in targeting the “undruggable” pathological proteins by traditional approaches.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

Thus, those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A hetero-bifunctional compound having the chemical structure:
PTM-L-VLM,
or a pharmaceutically acceptable salt or solvate thereof,
wherein:
(a) the PTM is a small molecule Kirsten ras sarcoma protein (KRas) targeting moiety that binds to KRas^{G12C}, and is represented by the chemical structure: 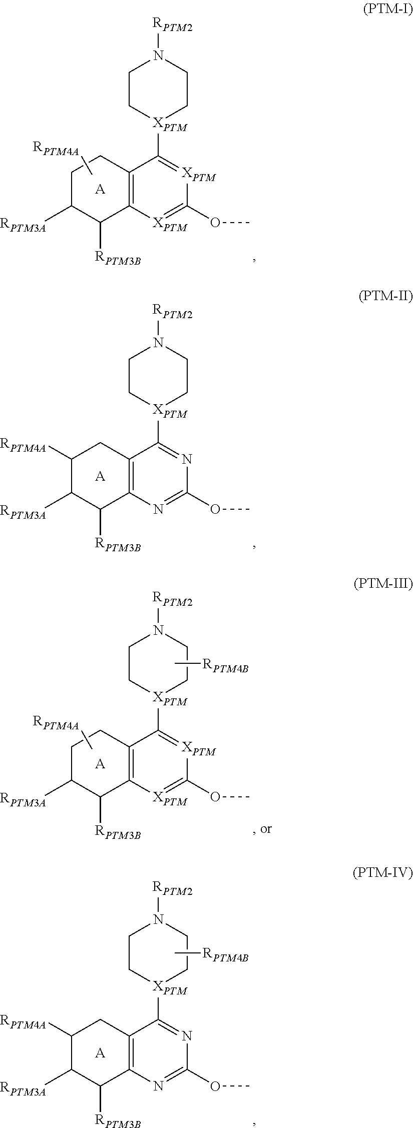
wherein:
the [symbol] of the PTM is the site of attachment to the L coupling the VLM to the PTM; 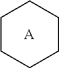
is an 6-membered aryl, 6-membered heteroaryl, or a 6-membered heterocycloalkyl, each optionally substituted with 1 or 2 halogens (e.g., Cl, F, or Br);
R_{PTM2} is —C(═O)C2-C4alkenyl, optionally substituted by a methyl, halogen (e.g., Cl, F, Br), amine (e.g., —NH₂, —NHCH₃, or —N(CH₃)₂), or a 3-6 membered heterocycloalkyl (e.g., a 6-membered heterocycloalkyl, a heterocycloalkyl having heteroatoms selected from O and N, or 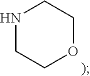
R_{PTM3A} is H, phenyl, pyridinyl, isoquinoline, or naphthalene 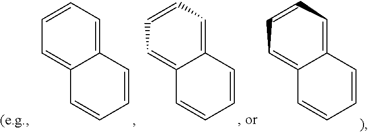
each optionally substituted by 1, 2, or 3 groups independently selected from OH, halogen (e.g., F, Cl, Br), amine (e.g., —NH₂, —NHCH₃, or —N(CH₃)₂), a linear or branched C1-C3 haloalkyl (e.g., a linear or branched C1-C3 fluoroalkyl or CF₃), —R_{PTM3C} 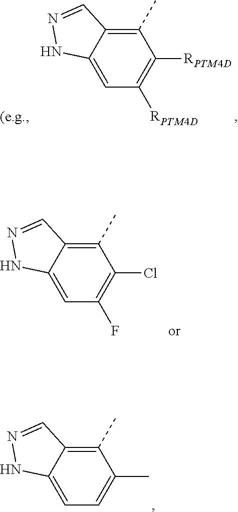
wherein each R_{PTM4D} is independently selected from a hydrogen, C1-C3 alkyl (e.g., methyl), or a halogen (e.g., F, Cl, Br)), or a linear or branched C1-C3 alkyl (e.g., methyl or ethyl);
R_{PTM3C} is an indazole optionally substituted by 1, 2, or 3 groups independently selected from OH, halogen (e.g., F, Cl, Br), or a linear or branched C₁-C₃ alkyl (e.g., methyl or ethyl);
R_{PTM3B} is H, halogen (e.g., Cl, F, Br), or —O—R_{PTM3C} 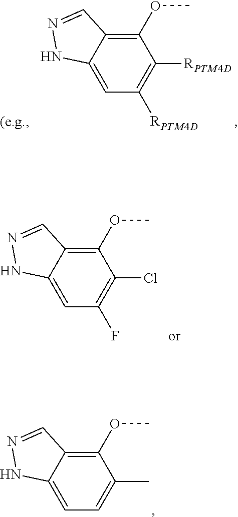
wherein each R_{PTM4D} is independently selected from a hydrogen, C1-C3 alkyl (e.g., methyl), or a halogen (e.g., F, Cl, Br));
R_{PTM4A} of PTM-I or PTM-III is absent (or H), or 1 or 2 independently selected halogen (e.g., Cl, F, Br);
R_{PTM4A} of PTM-II and PTM-IV is absent (or H) or a halogen (e.g., Cl, F, Br);
R_{PTM4B} is (1) absent (or H), (2) —CH₂—CH₂—CN or —CH₂—CN, or (3) 1 or 2 independently selected C₁-C₃ alkyl (e.g., methyl or ethyl); and
each X_{PTM} is individually a CH or N;
(b) the VLM is a small molecule E3 ubiquitin ligase binding moiety that binds a Von Hippel-Lindau (VHL) E3 ubiquitin ligase, and is represented by the chemical structure: 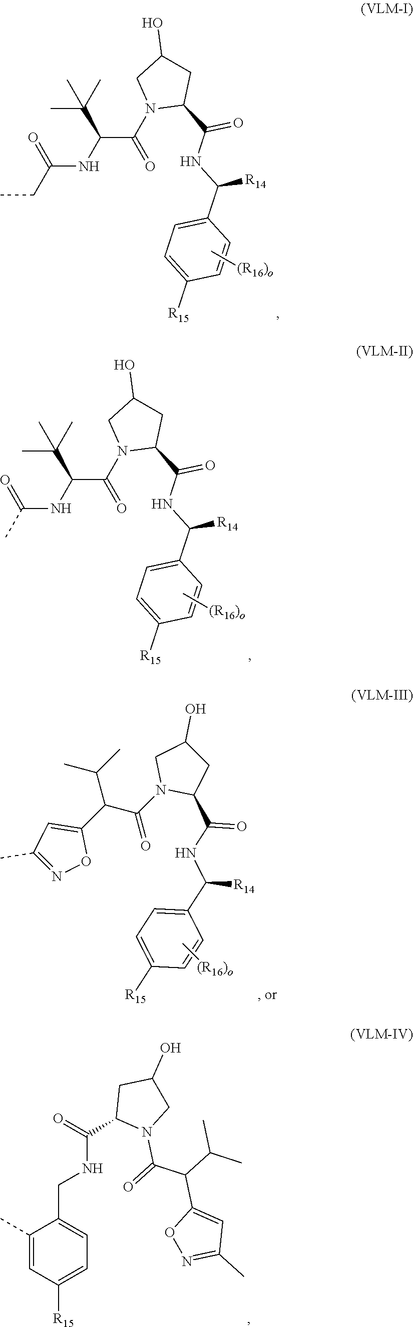
wherein:
the [symbol] of the VLM is the site of attachment to the L coupling the PTM to the VLM;
R₁₄ is H or a linear or branched C₁-C₃ alkyl (e.g., methyl);
R₁₅ is a CN or a 5-membered heteroaryl having one or two heteroatoms selected from N, S, and O, optionally substituted with a methyl 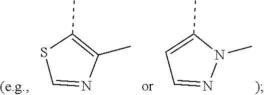
R₁₆ a halo, optionally substituted C1-C3 alkyl, optionally substituted C—C3 haloalkyl, hydroxy, optionally substituted C1-C3 alkoxy, or optionally substituted C1-C3 haloalkoxy; and
is an integer from 0-2 (e.g., 0, 1, or 2); and
(c) the L is a chemical linking moiety that covalently couples the VLM to the PTM.

2. The compound according to claim 1, wherein compound is represented by the chemical structure:

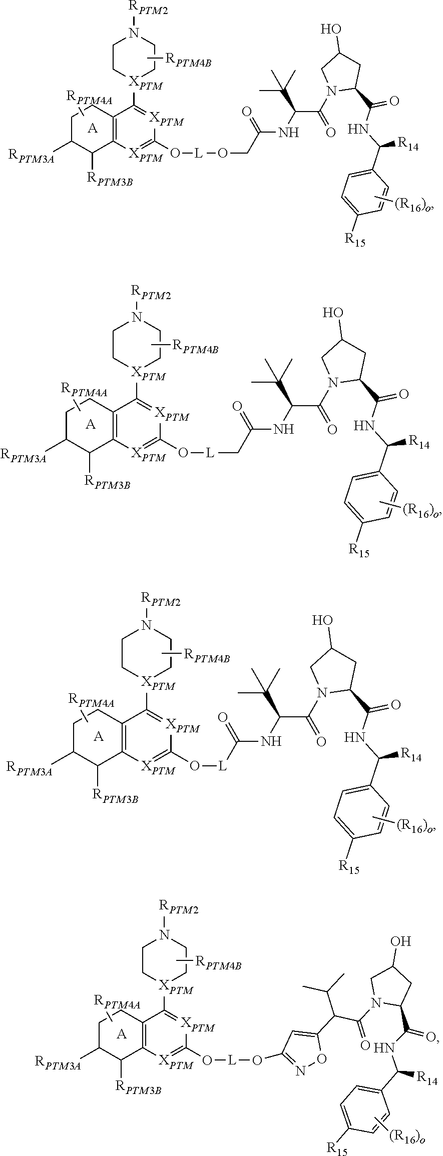

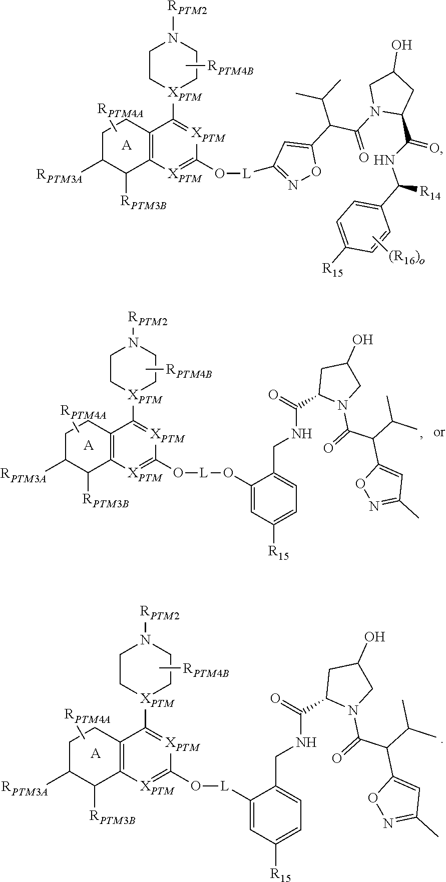

3. The compound according to claim 1, wherein the PTM is represented by the chemical structure: 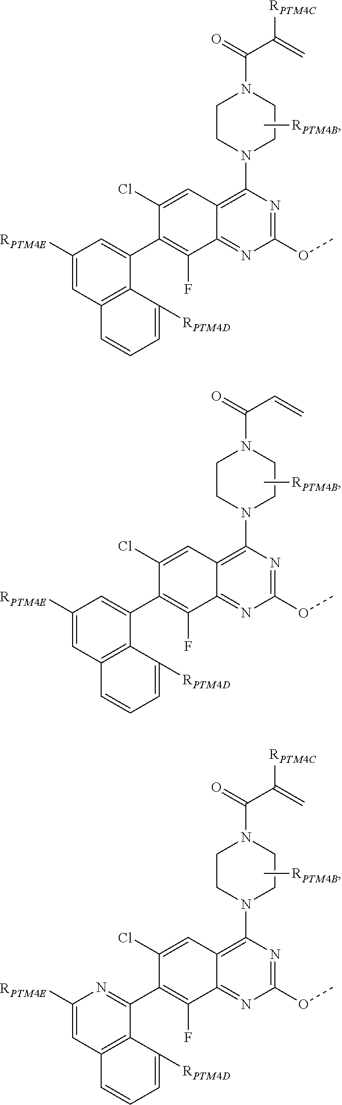 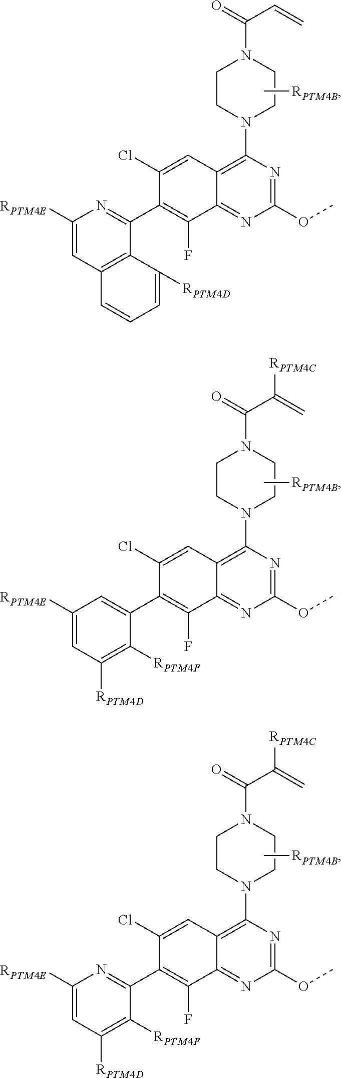 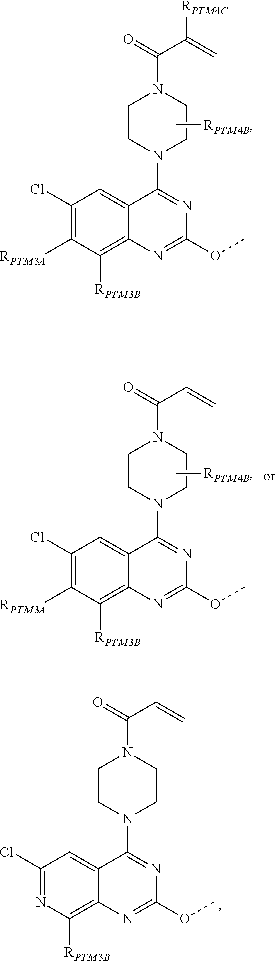
wherein:
the [symbol] of the PTM is the site of attachment to the chemical linking moiety coupling the VLM to the PTM;
R_{PTM3A} is indazole, optionally substituted by 1 or 2 groups independently selected from OH, methyl, and halogen (e.g., F, Cl, Br);
R_{PTM4B} is (1) H or absent, (2) —CH₂—CH₂—CN or —CH₂—CN, or (3) 1 or 2 independently selected C1-C3 alkyl (e.g., methyl or ethyl);
R_{PTM4D} is a hydrogen, C₁-C₃ alkyl (e.g., methyl), or a halogen (e.g., F, Cl, Br);
R_{PTM4C} is H or halogen (e.g., Cl, F, Br);
R_{PTM4E} is H, OH, or amine (e.g., —NH₂, or —NHCH₃);
R_{PTM4F} is a hydrogen, C1-C3 alkyl (e.g., methyl), C1-C3 haloalkyl (e.g., C1-C3 fluoroalkyl or CF₃), or a halogen (e.g., F, Cl, Br); and
R_{PTM3B} is —O-indazole, optionally substituted by 1 or 2 groups independently selected from OH, methyl, and halogen (e.g., F, Cl, Br).

4. The compound according to claim 1, wherein the PTM is represented by the chemical structure: 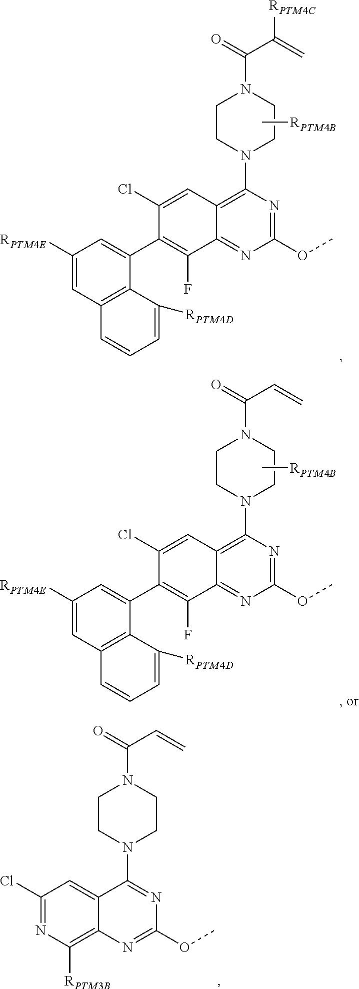
wherein:
the [symbol] of the PTM is the site of attachment to the chemical linking moiety coupling the VLM to the PTM;
R_{PTM4B} is (1) H or absent, (2) —CH₂—CH₂—CN or —CH₂—CN, or (3) 1 or 2 independently selected C₁-C₃ alkyl (e.g., methyl or ethyl);
R_{PTM4D} is a hydrogen, C1-C3 alkyl (e.g., methyl), or a halogen (e.g., F, Cl, Br);
R_{PTM4C} is H or halogen (e.g., Cl, F, Br);
R_{PTM4E} is H or OH; and
R_{PTM3B} is —O-indazole, optionally substituted by 1 or 2 groups independently selected from OH, and halogen (e.g., F, Cl, Br).

5. The compound according to claim 1, wherein the PTM has the chemical structure: 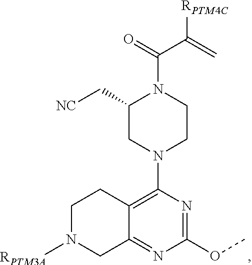
wherein:
the [symbol] of the PTM is the site of attachment to the chemical linking moiety coupling the VLM to the PTM;
R_{PTM3A} is: 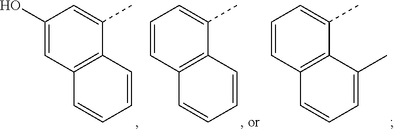
and
R_{PTM4C} is H or F.

6. The compound according to claim 1, wherein the PTM has the chemical structure: 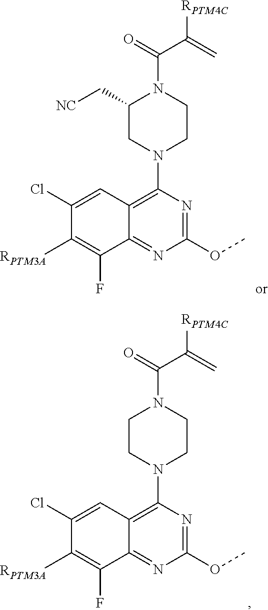
wherein:
the [symbol] of the PTM is the site of attachment to the chemical linking moiety coupling the VLM to the PTM;
R_{PTM3A} is: 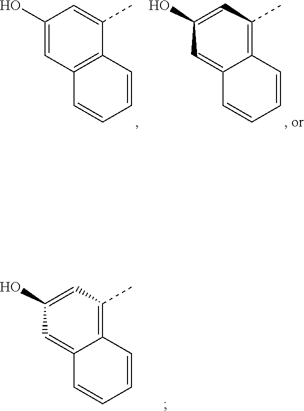
and
R_{PTM4C} is H or F.

7. The compound according to claim 3, wherein:
(a) the PTM is selected from: 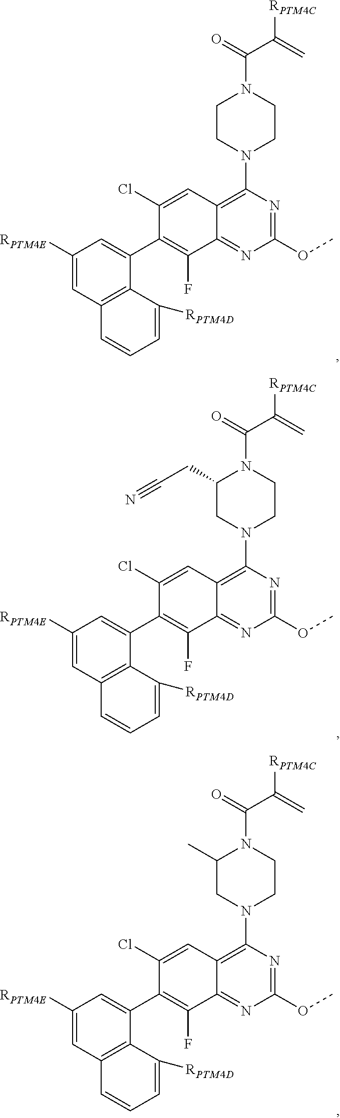 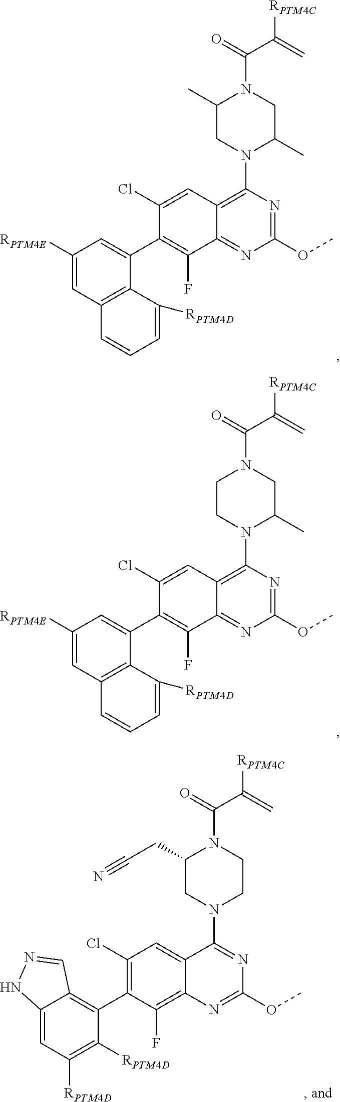 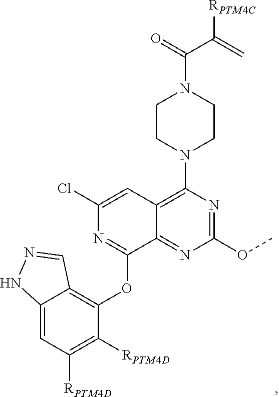
wherein:
the [symbol] of the PTM is the site of attachment to the chemical linking moiety coupling the VLM to the PTM;
each R_{PTM4D} is independently selected from a hydrogen, C1-C3 alkyl (e.g., methyl), and a halogen (e.g., F, Cl, Br); or
(b) the PTM is selected from: 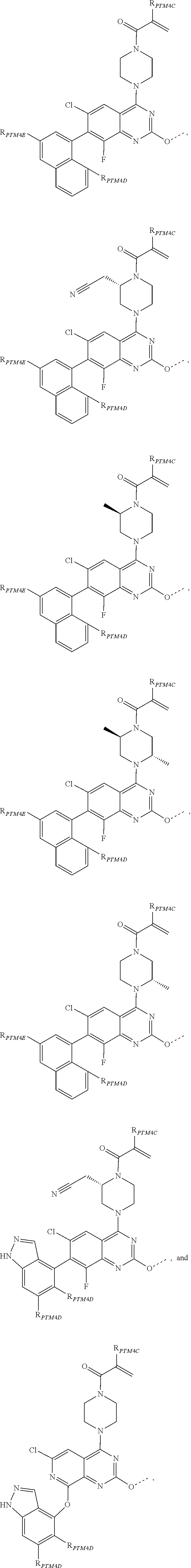
wherein:
the [symbol] of the PTM is the site of attachment to the chemical linking moiety coupling the VLM to the PTM;
each R_{PTM4D} is independently selected from a hydrogen, C1-C3 alkyl (e.g., methyl), and a halogen (e.g., F, Cl, Br).

8. The compound according to claim 4, wherein:
(a) the PTM is selected from: 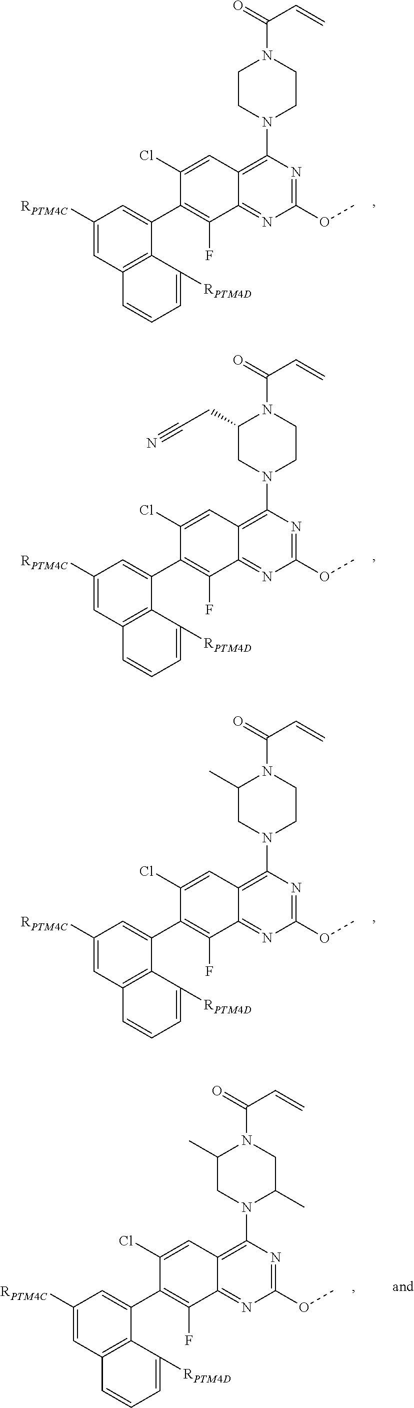 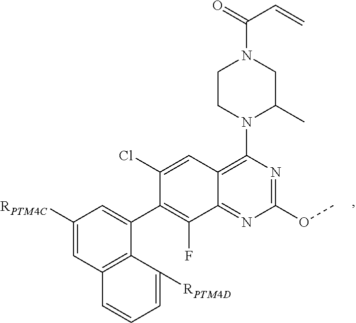
wherein the [symbol] of the PTM is the site of attachment to the chemical linking moiety coupling the VLM to the PTM; or
(b) the PTM is selected from: 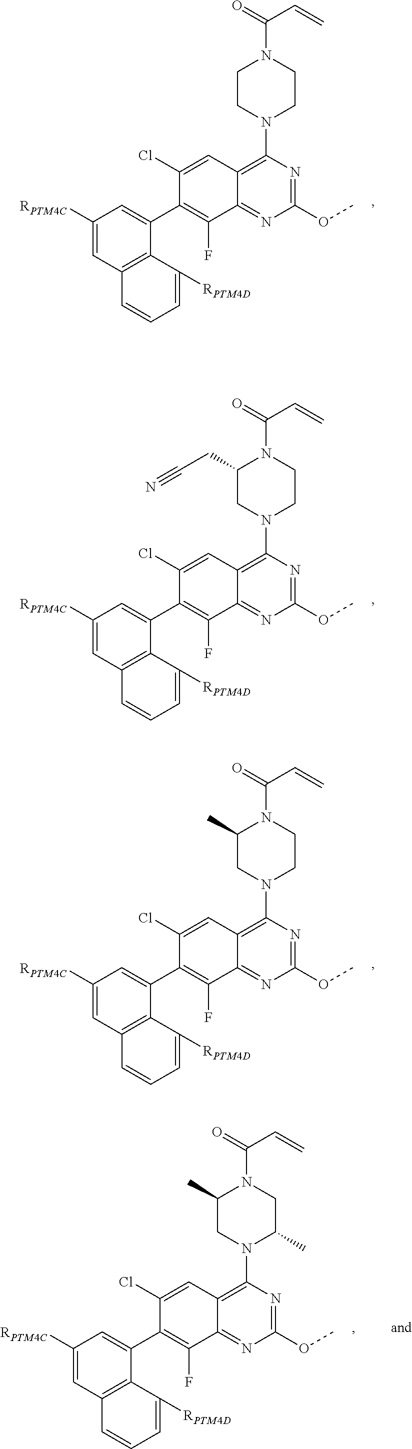 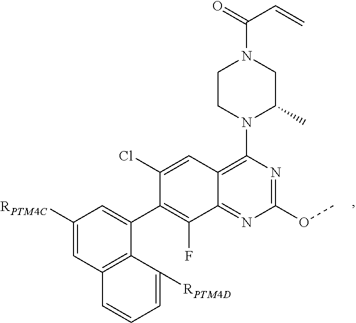
wherein the [symbol] of the PTM is the site of attachment to the chemical linking moiety coupling the VLM to the PTM.

9. The compound according to claim 1, wherein the PTM is represented by the chemical structure: 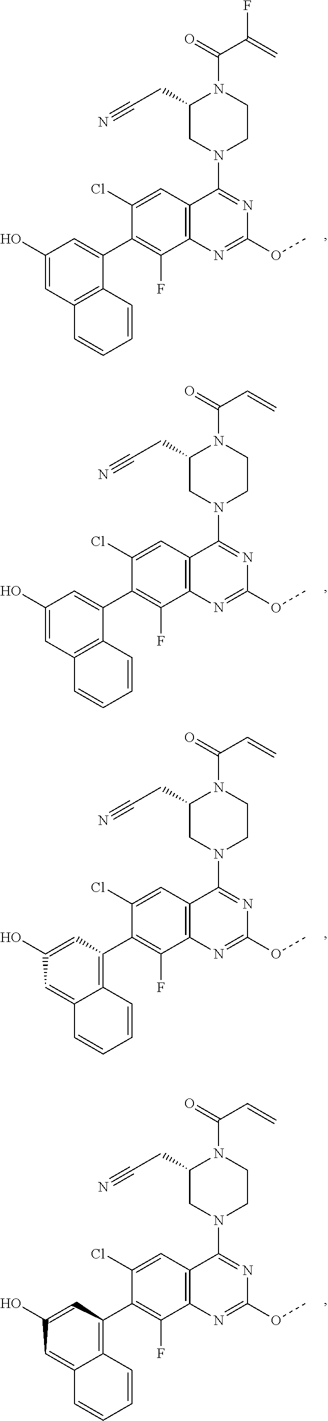 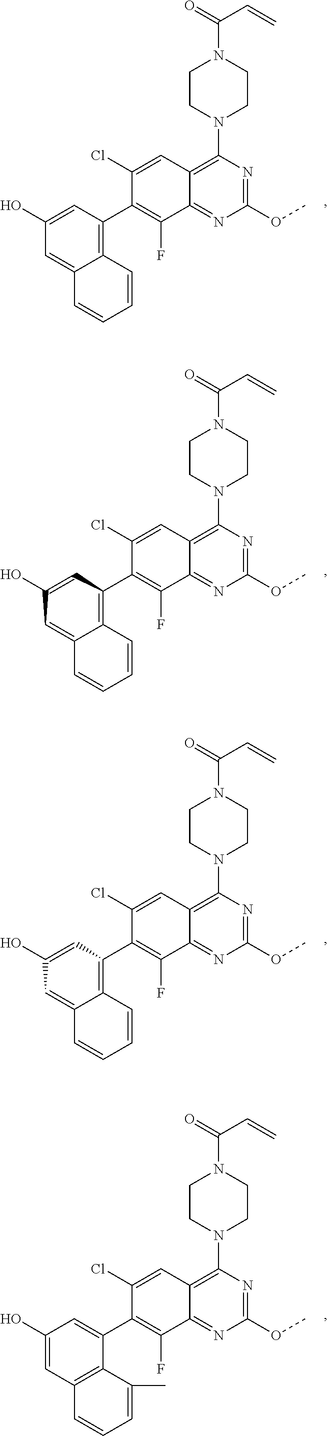 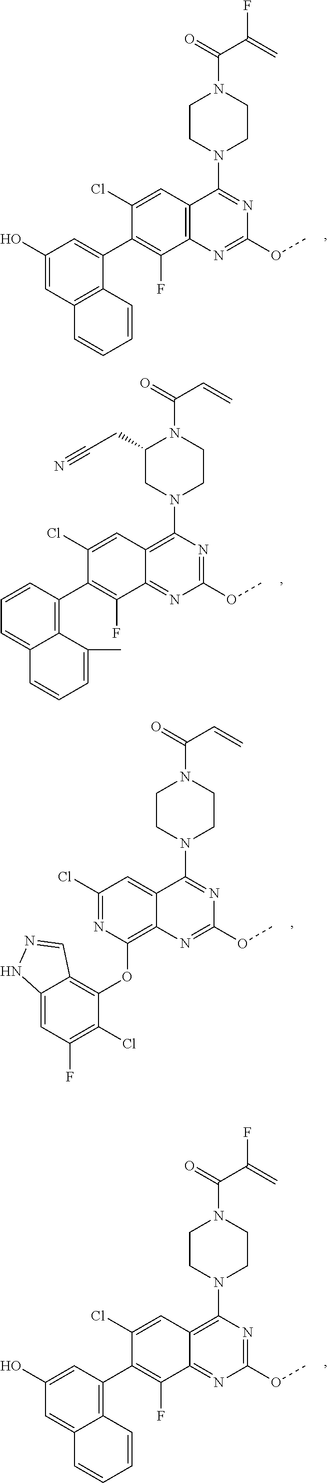 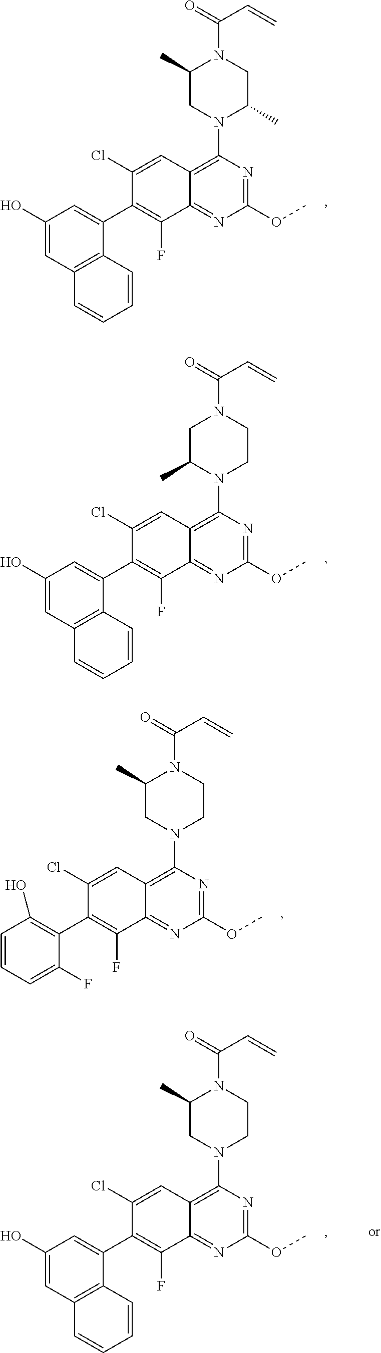 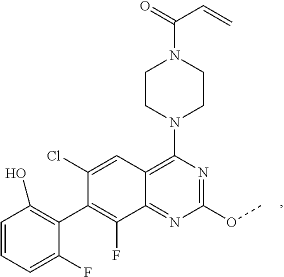
wherein the [symbol] of the PTM is the site of attachment to the chemical linking moiety coupling the VLM to the PTM.

10. The compound according to claim 1, wherein the PTM is represented by the chemical structure: 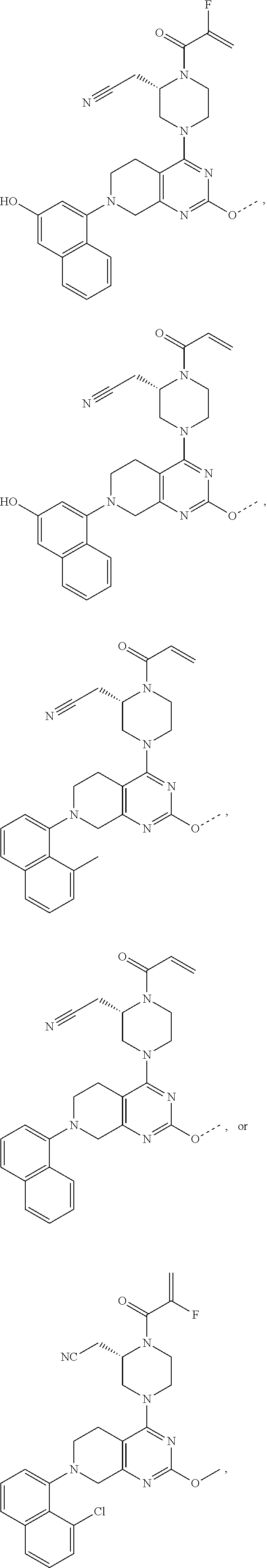
wherein the [symbol] of the PTM is the site of attachment to the chemical linking moiety coupling the VLM to the PTM.

11. The compound according to claim 1, wherein the PTM is represented by the chemical structure: 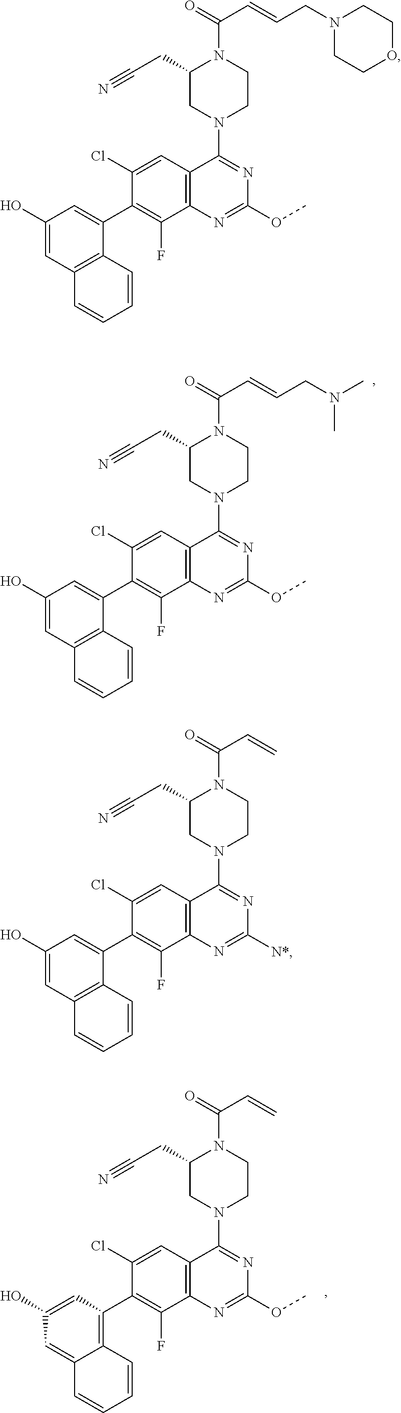 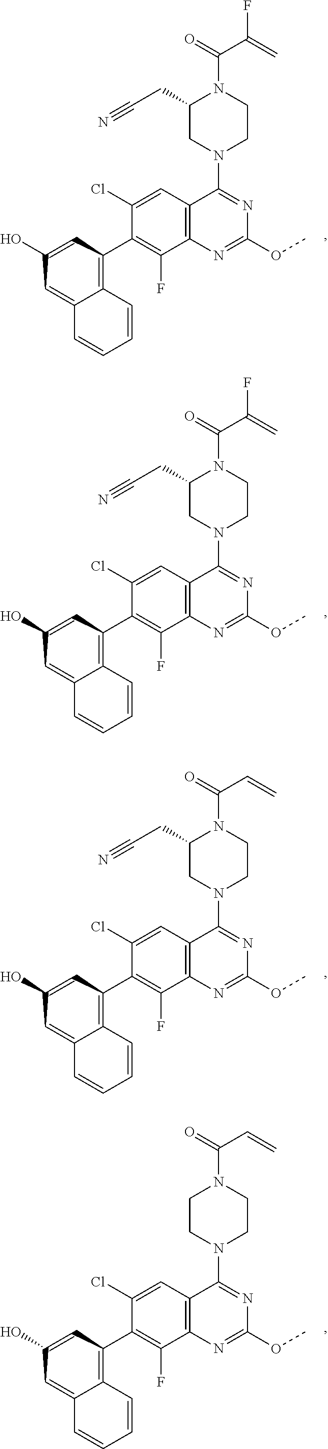 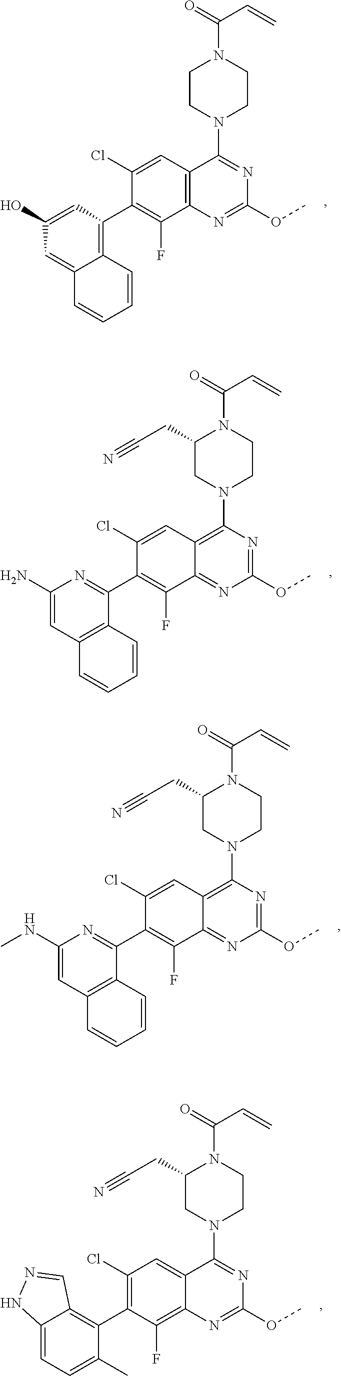 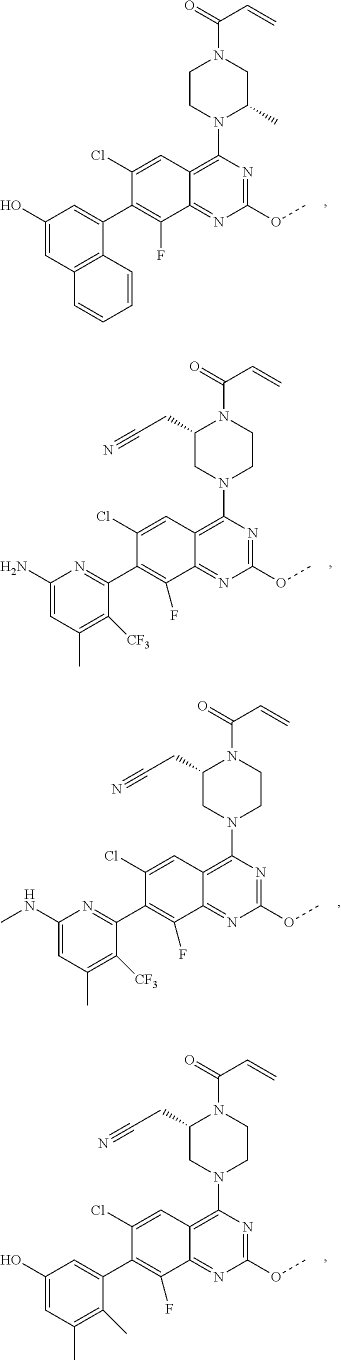
wherein * denotes an atom that is the site of attachment with the chemical linking moiety or an atom that is shared with the chemical linking moiety, and the [symbol] of the PTM is the site of attachment to the chemical linking moiety coupling the VLM to the PTM.

12. The compound according to claim 1, wherein the VLM has a chemical structure represented by: 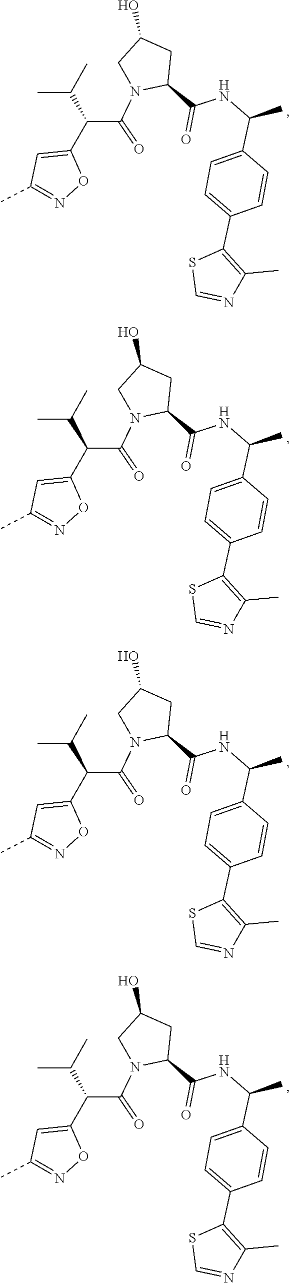 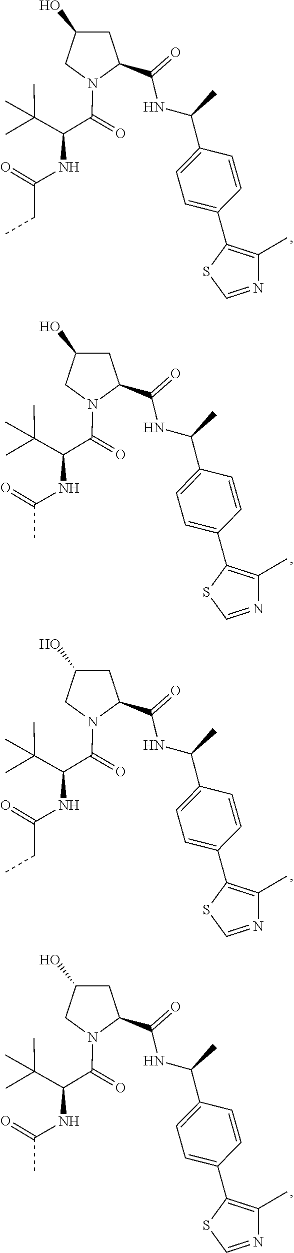 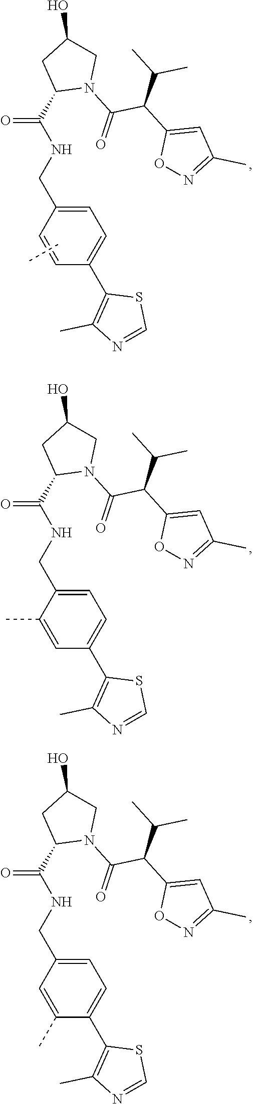 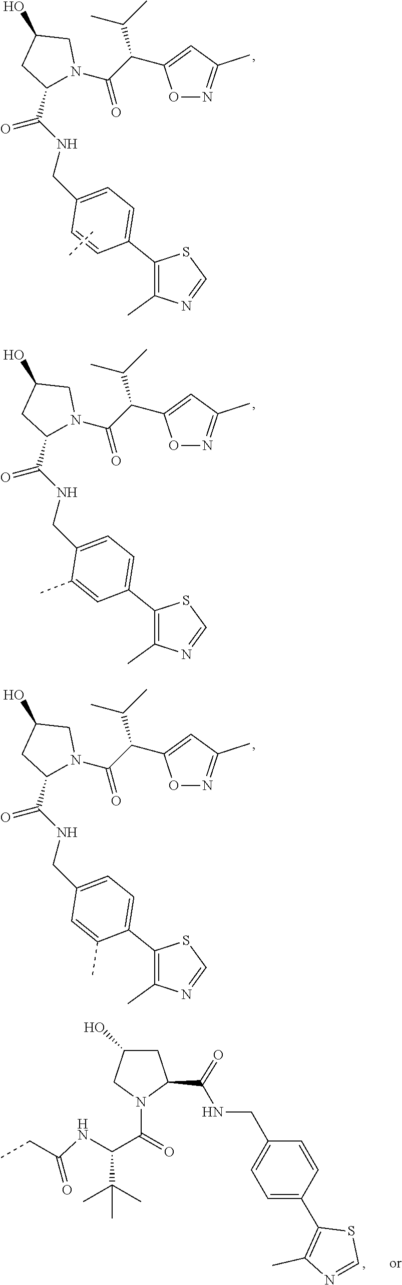 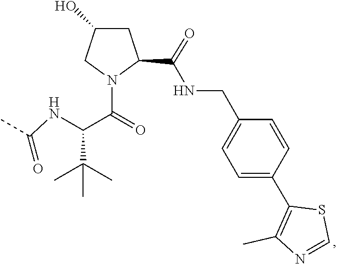
wherein the [symbol] of the VLM is the site of attachment to the chemical linking moiety coupling the PTM to the VLM.

13. The compound according to claim 1, wherein the VLM has a chemical structure represented by: 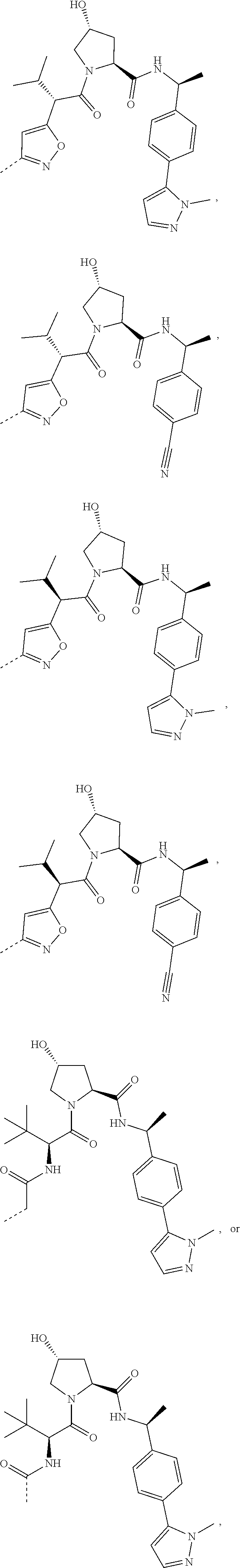
wherein the [symbol] of the VLM is the site of attachment to the chemical linking moiety coupling the PTM to the VLM.

14. The compound according to claim 1, wherein the chemical linking moiety comprises the following chemical structure: 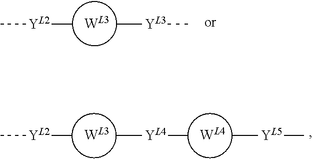
wherein:
the [symbol] of the chemical linking moiety is the site of attachment to the VLM or the PTM;
Y^{L2} is a bond, or a unsubstituted or substituted linear or branched C1-C4 alkyl (e.g., optionally substituted with a halogen, C1-3 alkyl, methyl, or ethyl);
W^{L3} is a 3-7 membered ring (e.g., 4-6 membered cycloalkyl or heterocycloalkyl) or an 8-12 membered spirocyclic, each with 0-4 heteroatoms (e.g., 0-4 heteroatoms independently selected from N, O, and S) and optionally substituted with halogen or methyl;
Y^{L3} is a bond or a C1-C32 alkyl (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, or C₃₂ alkyl), wherein one or more C atoms are optionally replaced with 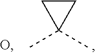
or NH, and each carbon is optionally substituted with a halogen, ═O, a methyl or ethyl, and each nitrogen is optionally substituted with a methyl or ethyl;
Y_{L4} is bond, O, or an unsubstituted or substituted linear or branched C1-C6 alkyl, wherein one or more carbons are optionally replaced O, NH, or NCH₃, and optionally substituted with a halogen or methyl;
W^{L4} is a 3-8 membered ring (e.g., 4-6 membered cycloalkyl or heterocycloalkyl, or 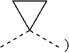
or a 5-8 member spirocyclic, each with 0-4 heteroatoms (e.g., 0-4 heteroatoms independently selected from N, O, and S) and optionally substituted with halogen (e.g., F, Cl, Br), or methyl; and
Y^{L5} is a bond or an unsubstituted or substituted C1-C6 alkyl, where one or more C atoms are optionally replaced with O and optionally substituted with a halo (e.g., F, Cl, Br), or methyl.

15. The compound according to claim 1, wherein the chemical linking moiety has a chemical structure represented by: 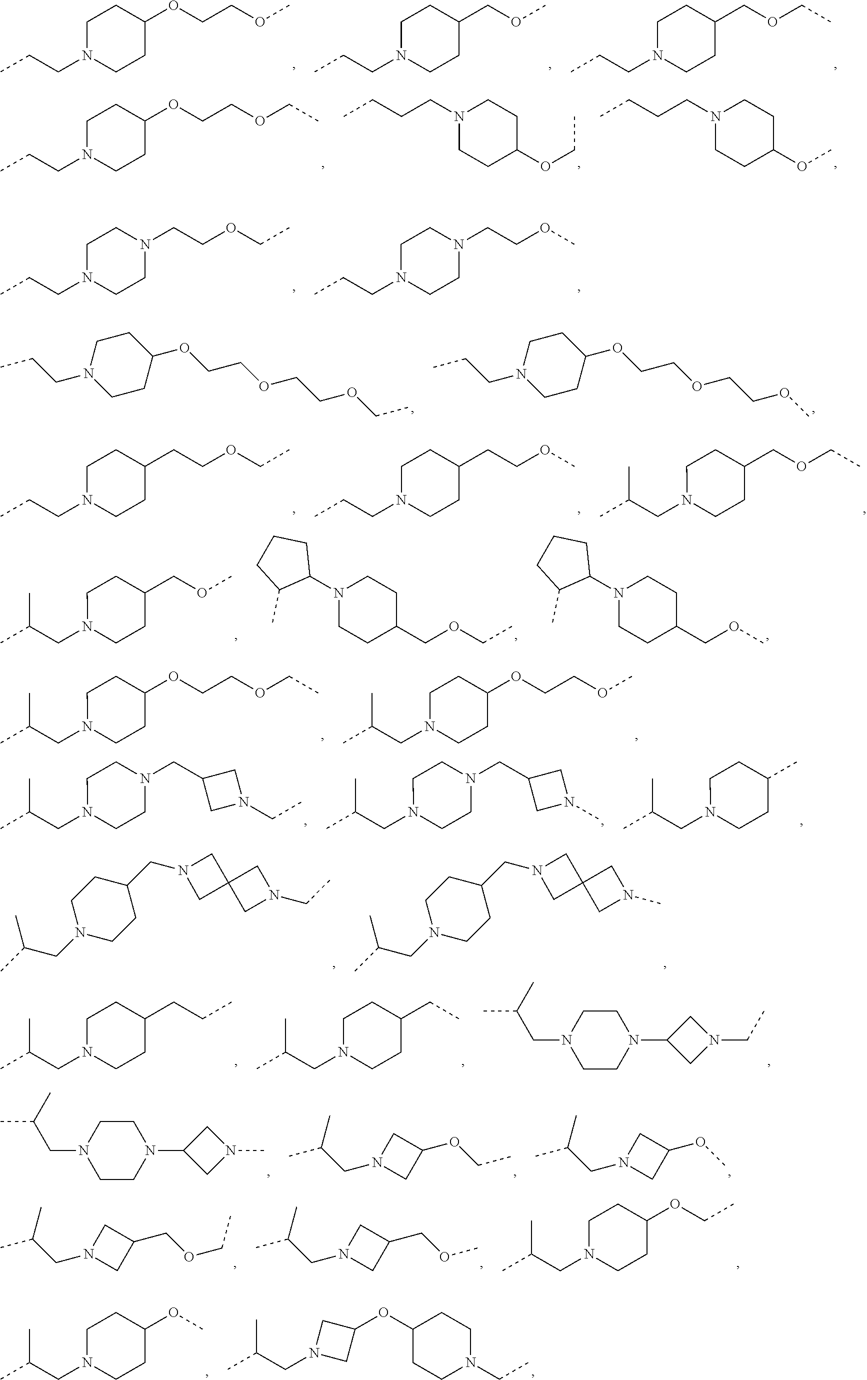 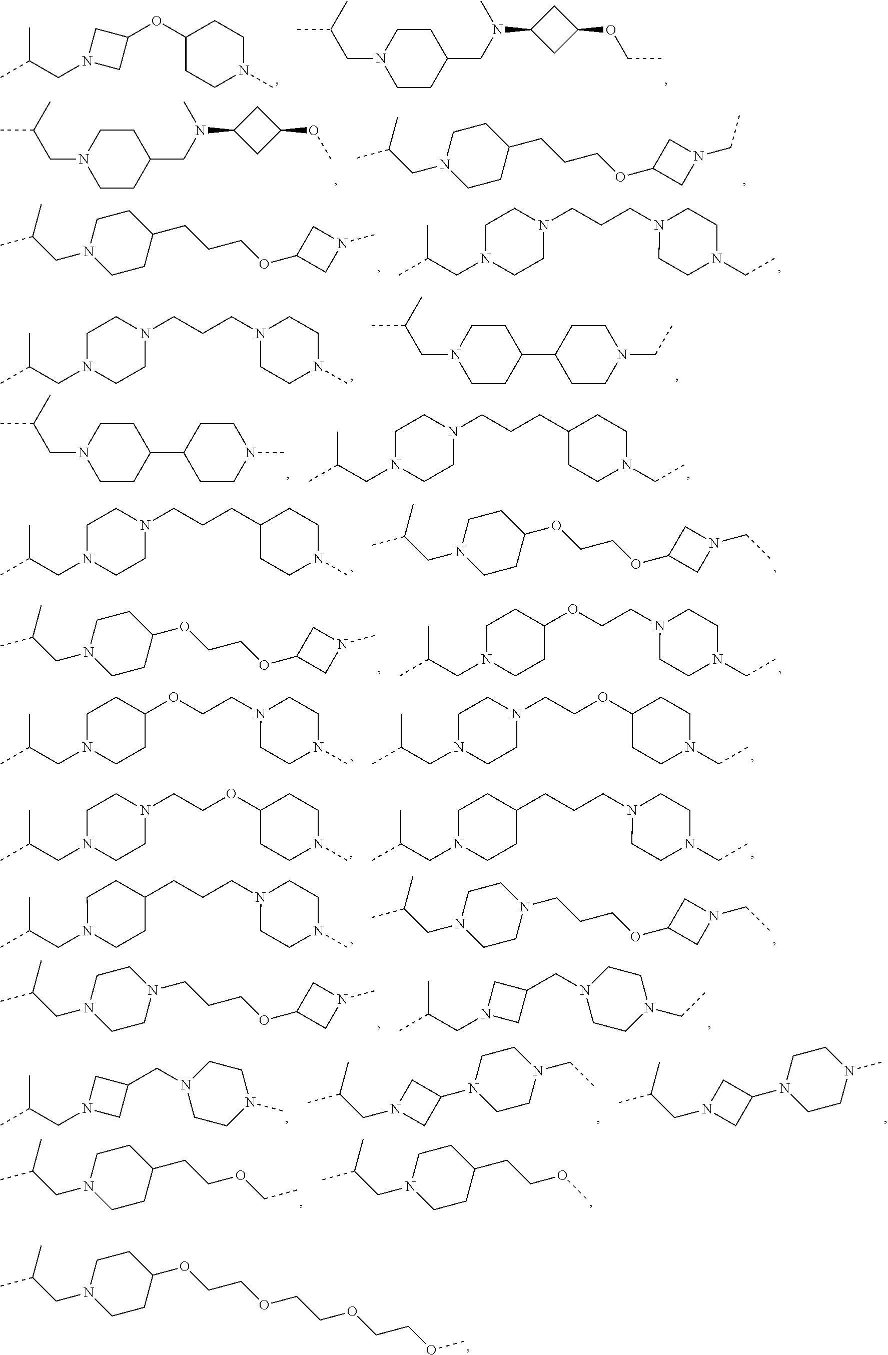 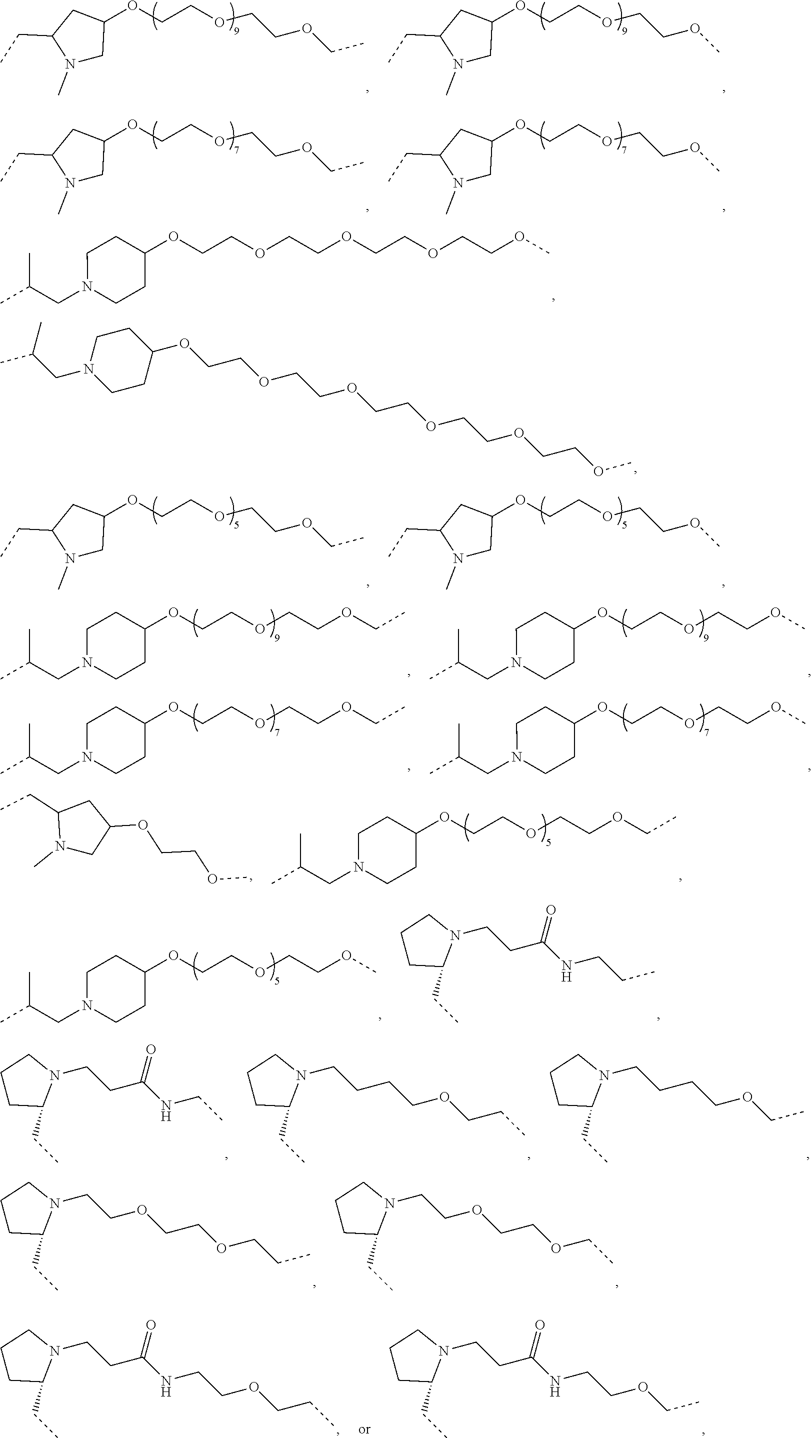
wherein the [symbol] of the chemical linking moiety indicates the point of attachment with the PTM or the VLM.

16. The compound according to claim 1, wherein the chemical linking moiety has a chemical structure represented by: 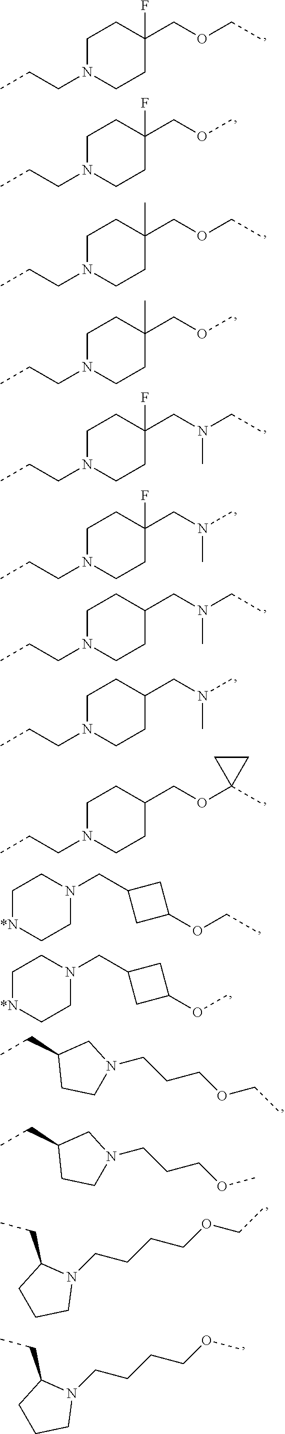 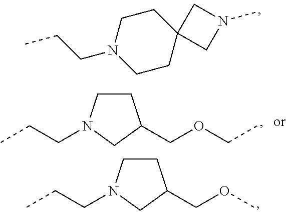
wherein the [symbol] of the chemical linking moiety indicates the point of attachment with the PTM or the VLM.

17. The compound according to claim 1, wherein the linker (L) is represented by: 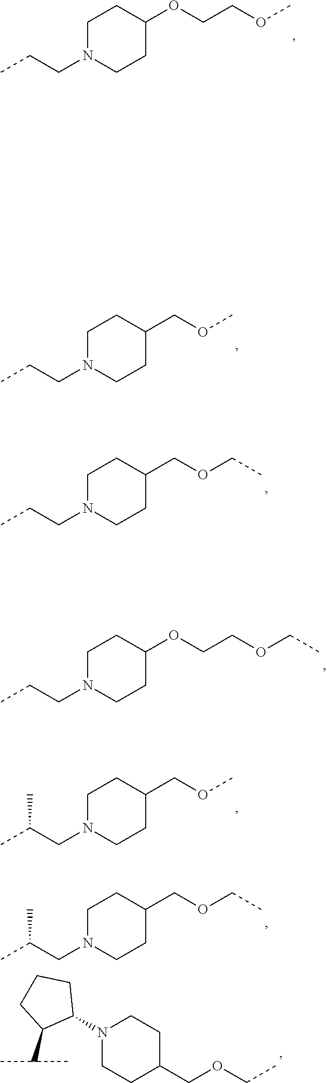 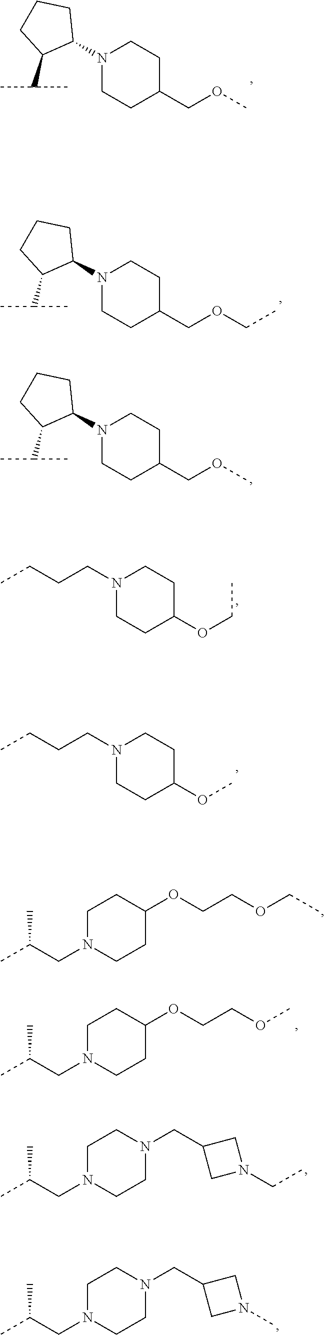 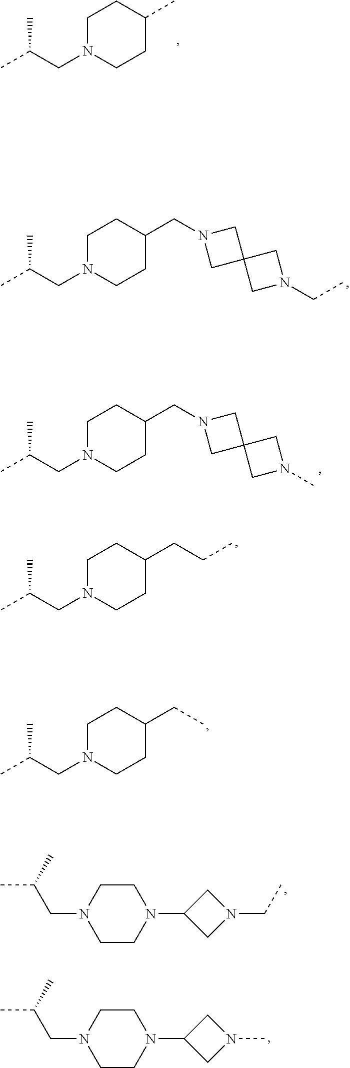 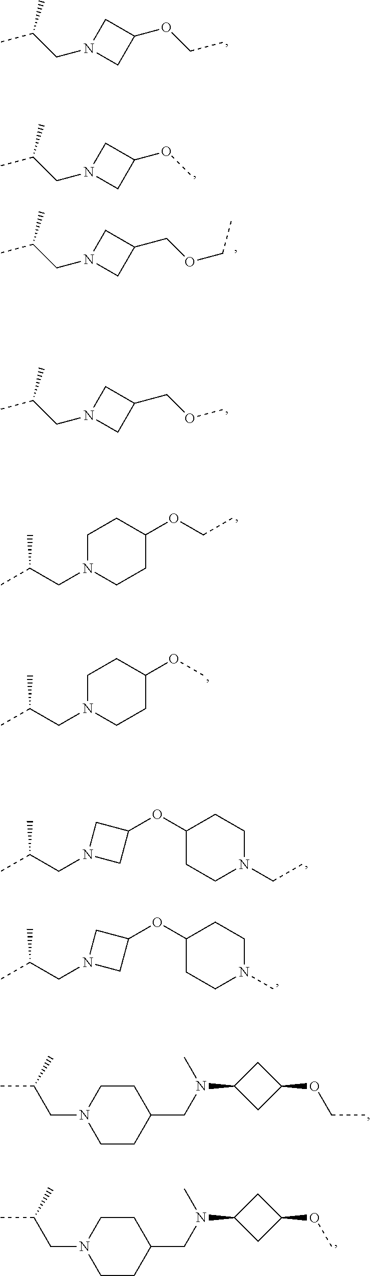 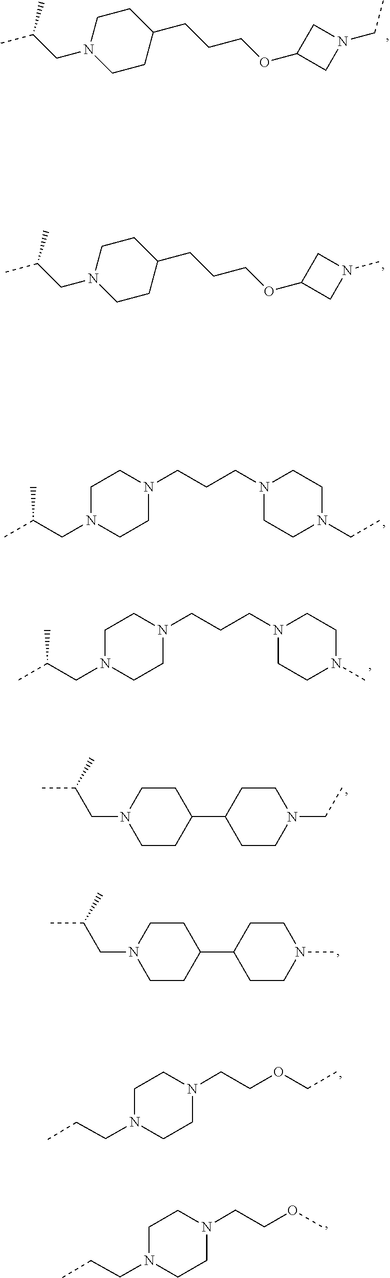 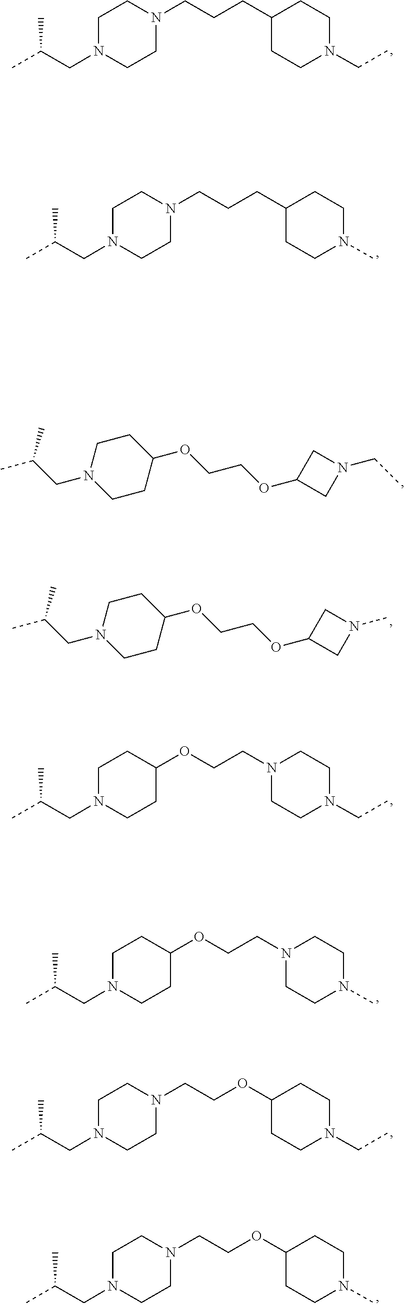 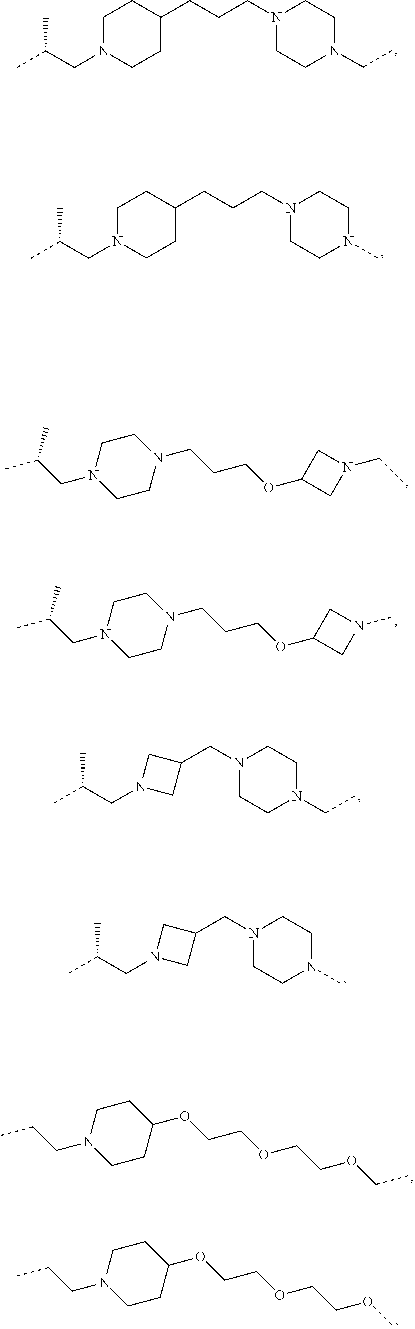 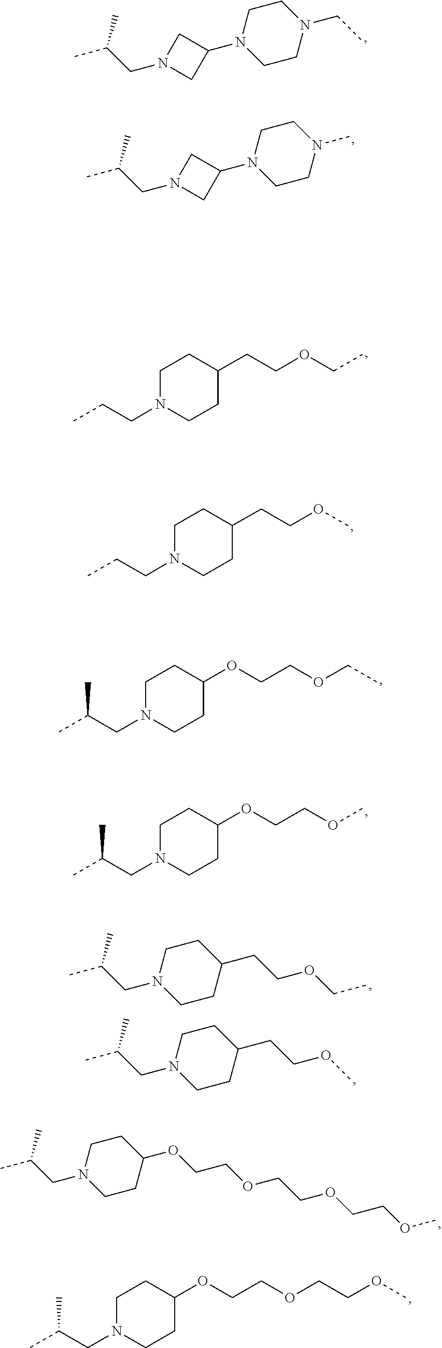 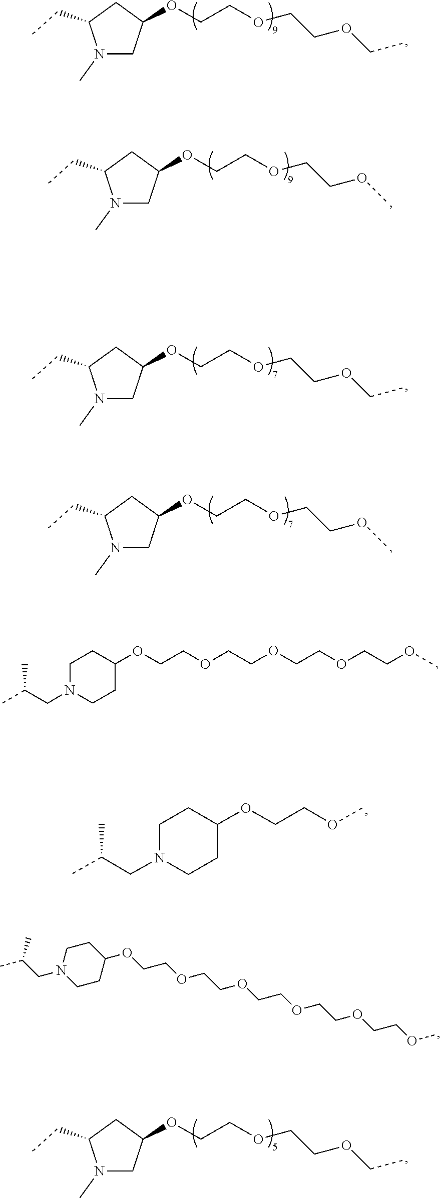 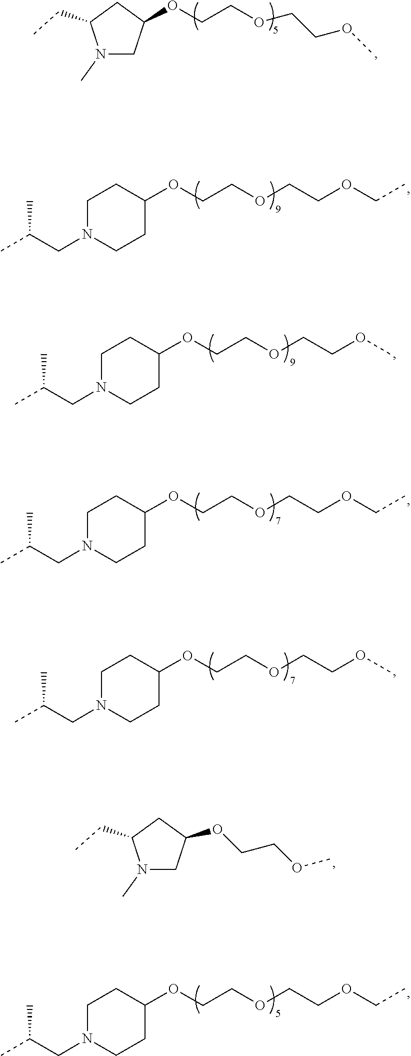 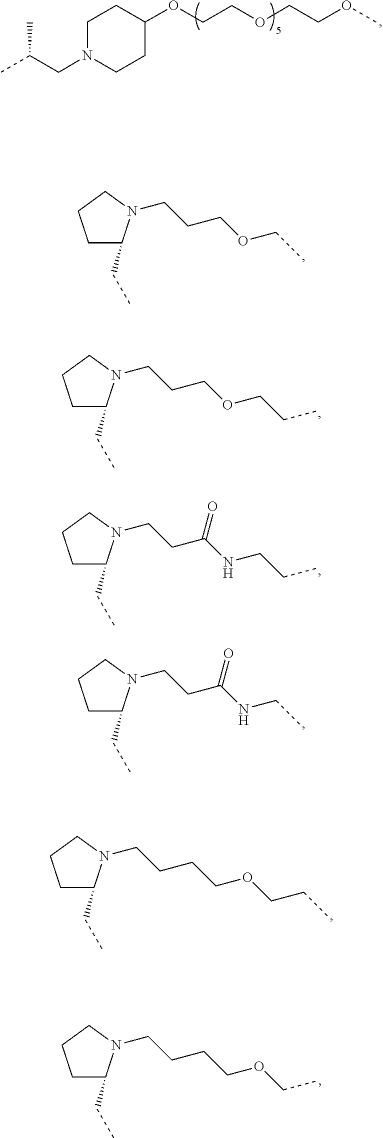 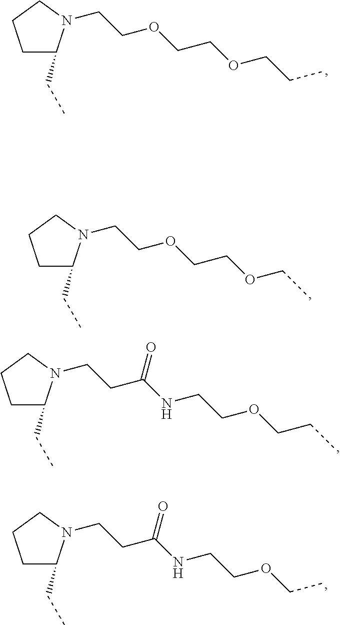
wherein the [symbol] of the chemical linking moiety indicates the point of attachment with the PTM or the VLM.

18. The compound according to claim 1, wherein the linker (L) is represented by: 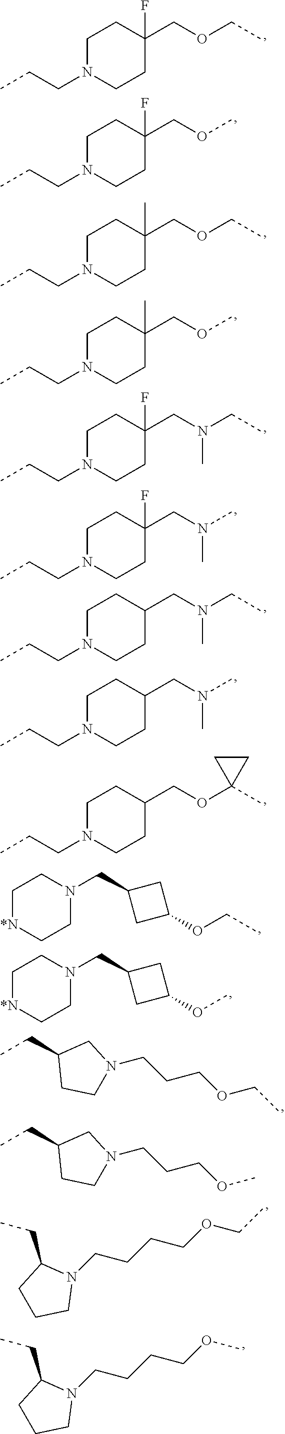 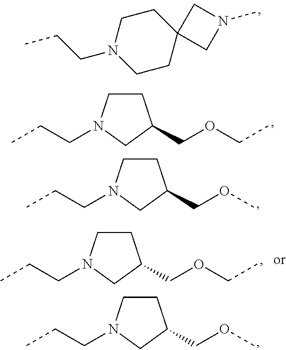
wherein the [symbol] of the chemical linking moiety indicates the point of attachment with the PTM or the VLM.

19. The compound according to claim 1, wherein at least one of:
the PTM is a PTM selected from a compound of Table 7;
the VLM is a VLM selected from a compound of Table 7; and
the L is an L selected from a compound of Table 7.

20. The compound according to claim 1, wherein the compound is selected from the group consisting of compounds 1-94 of Table 7:

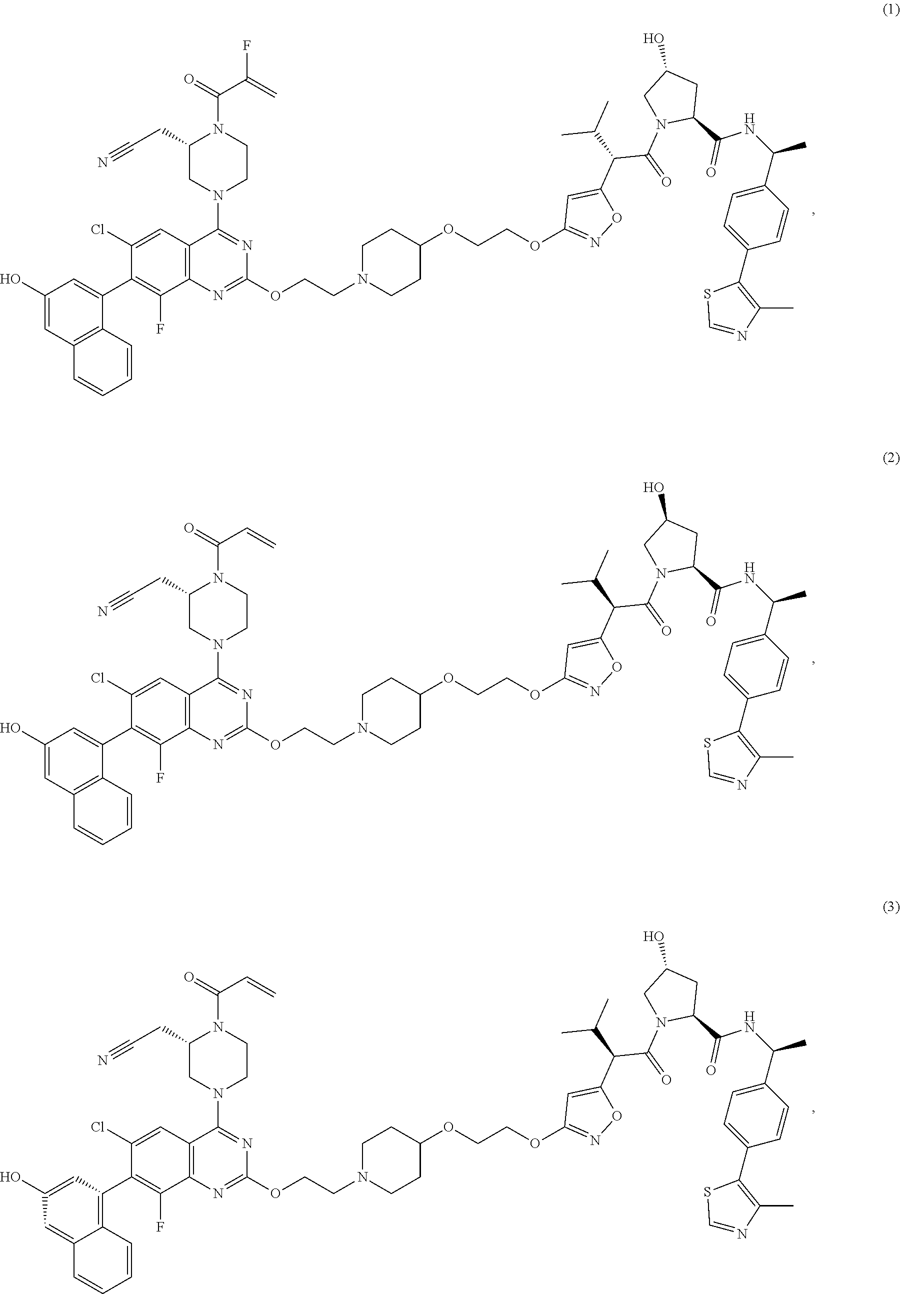

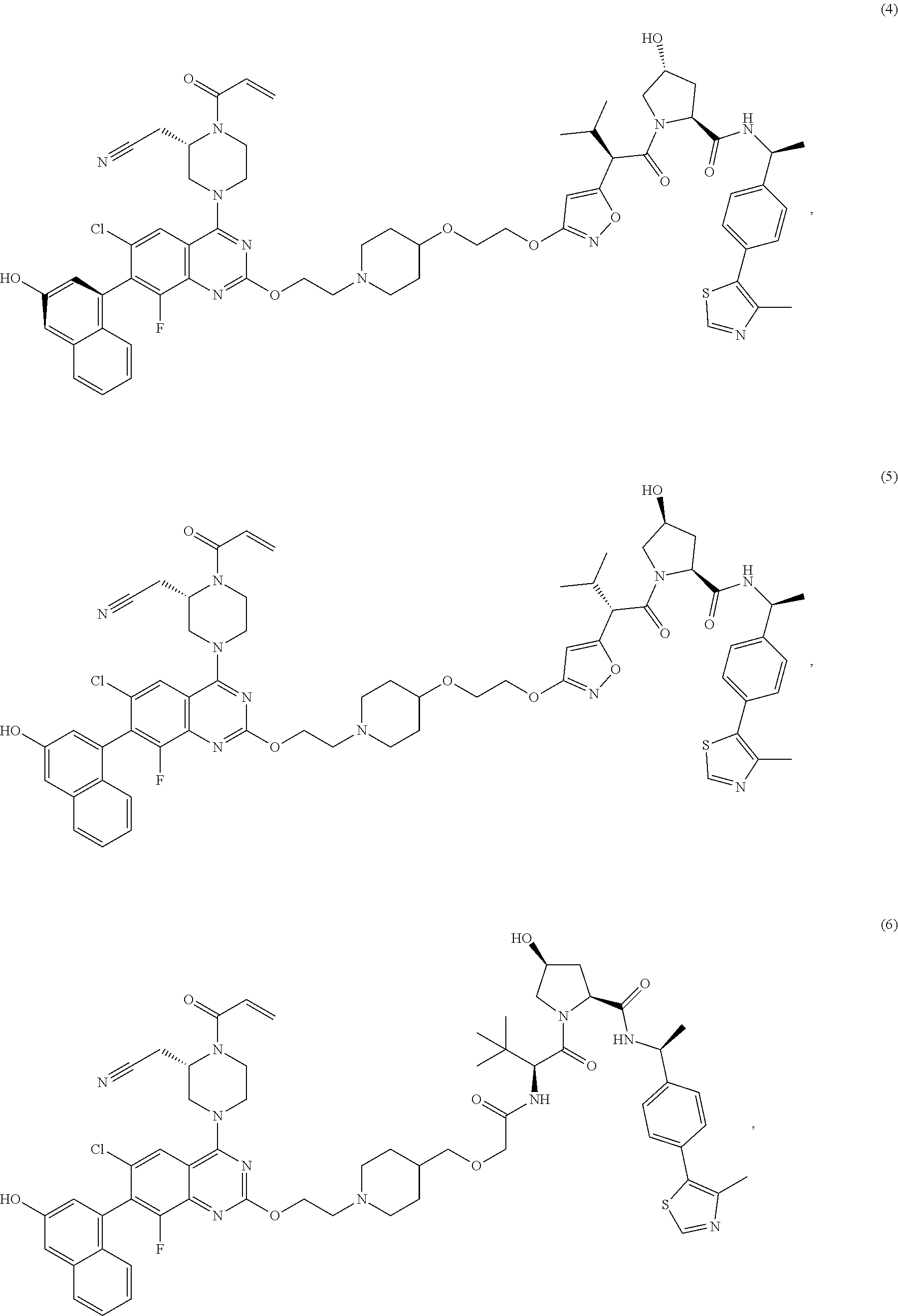

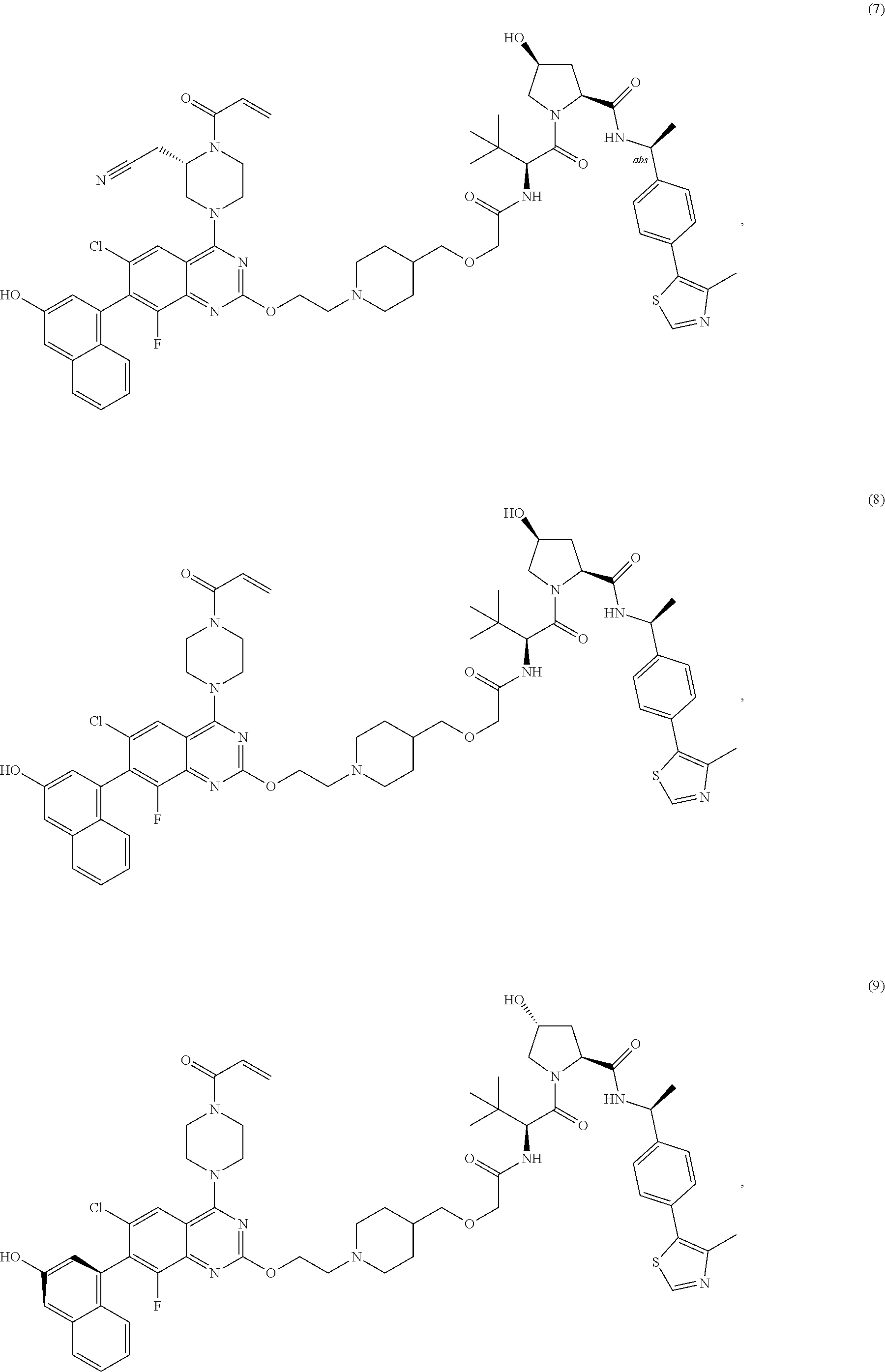

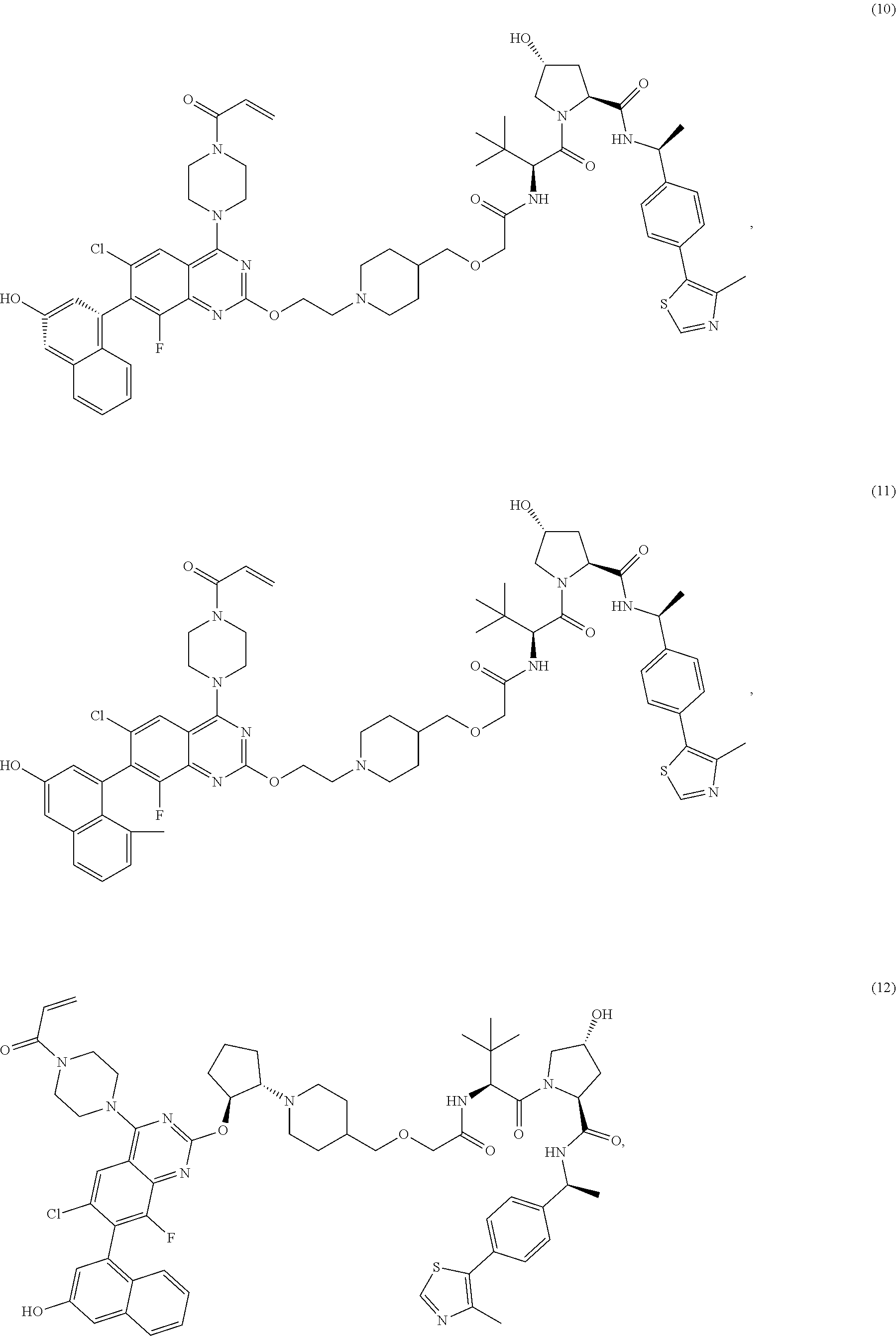

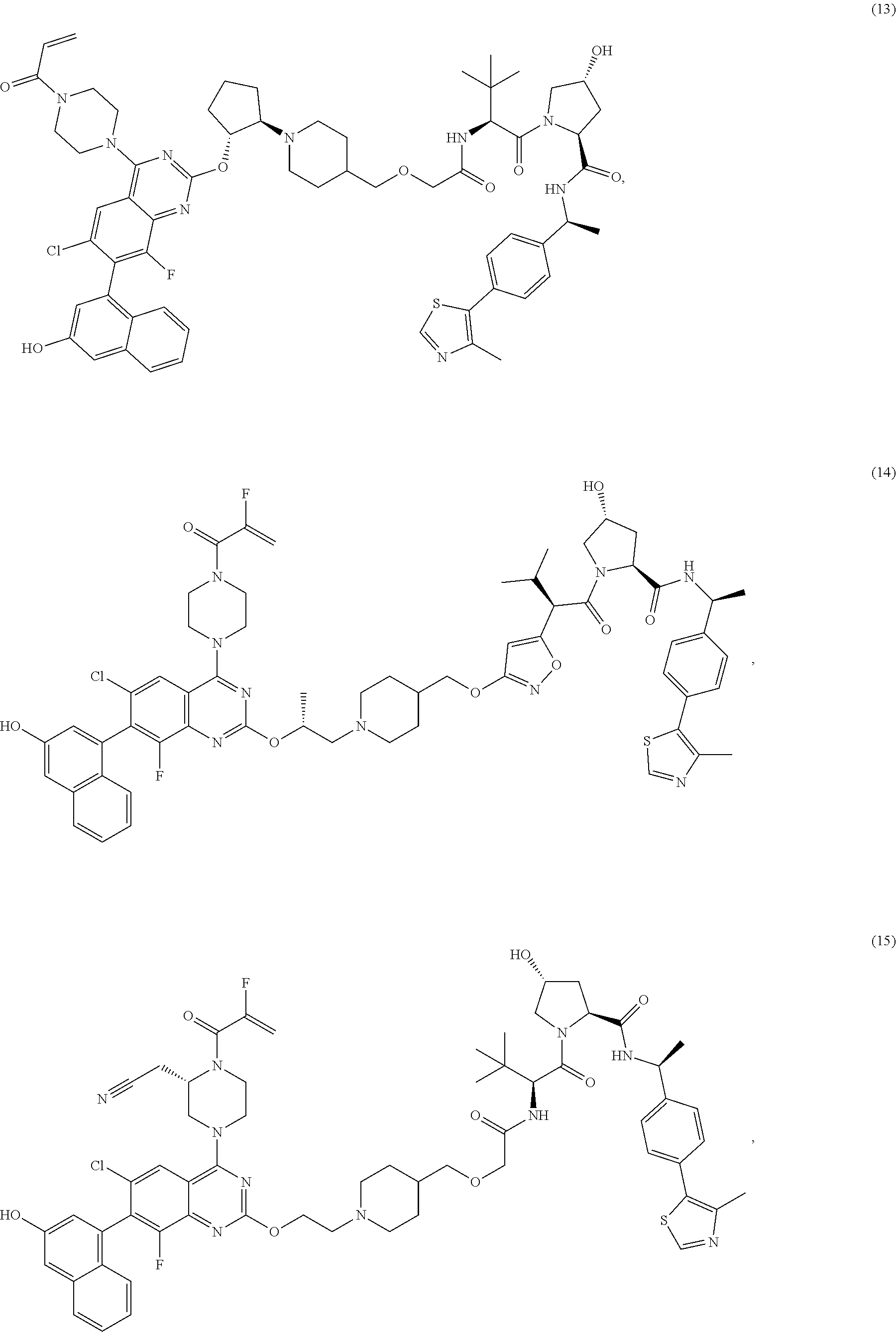

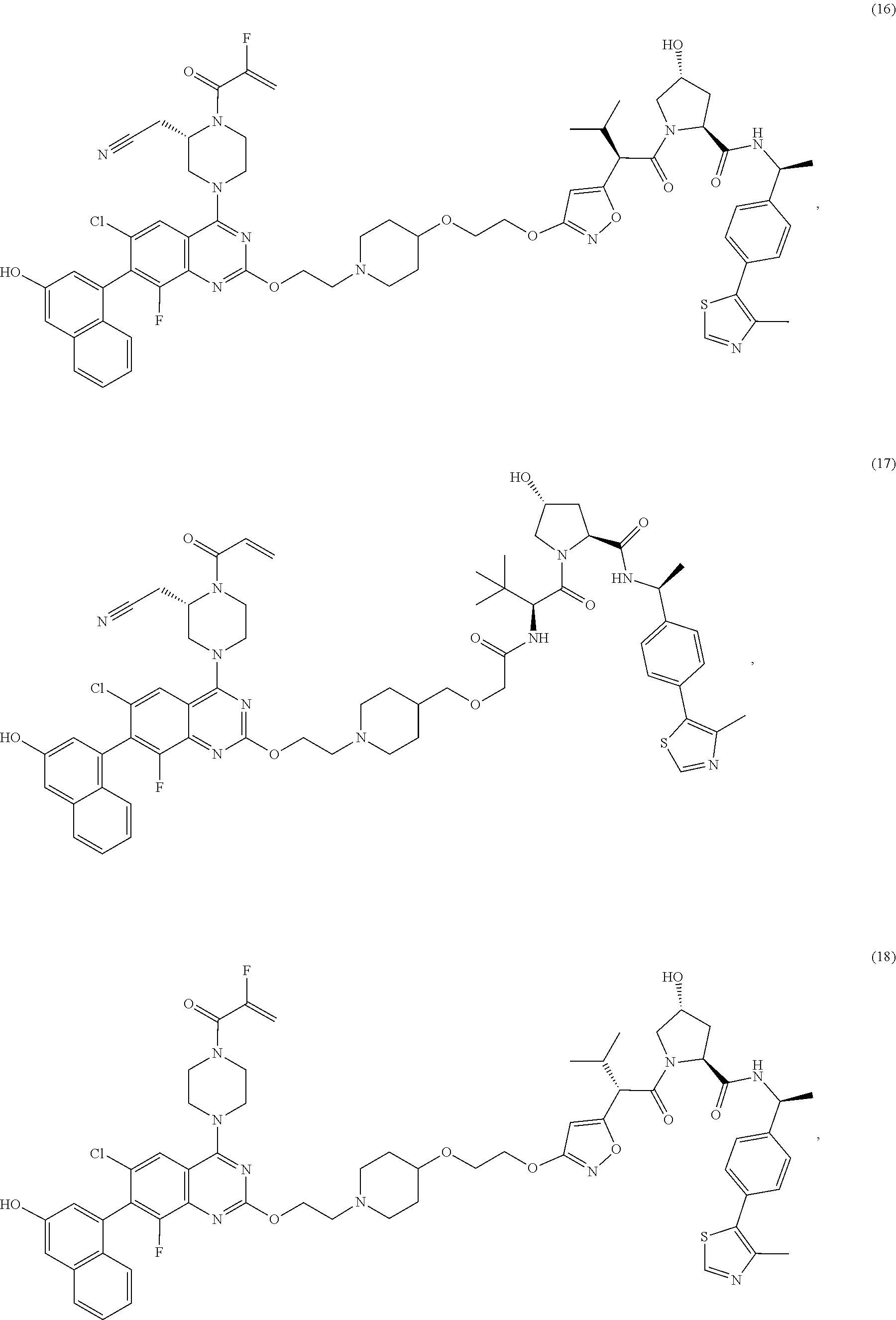 [SMILES: C=C(F)C(=O)N1CCN(c2nc(OCCN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N ; C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@@H]1CC#N ; C=C(F)C(=O)N1CCN(c2nc(OCCN3CCC(OCCOc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1]

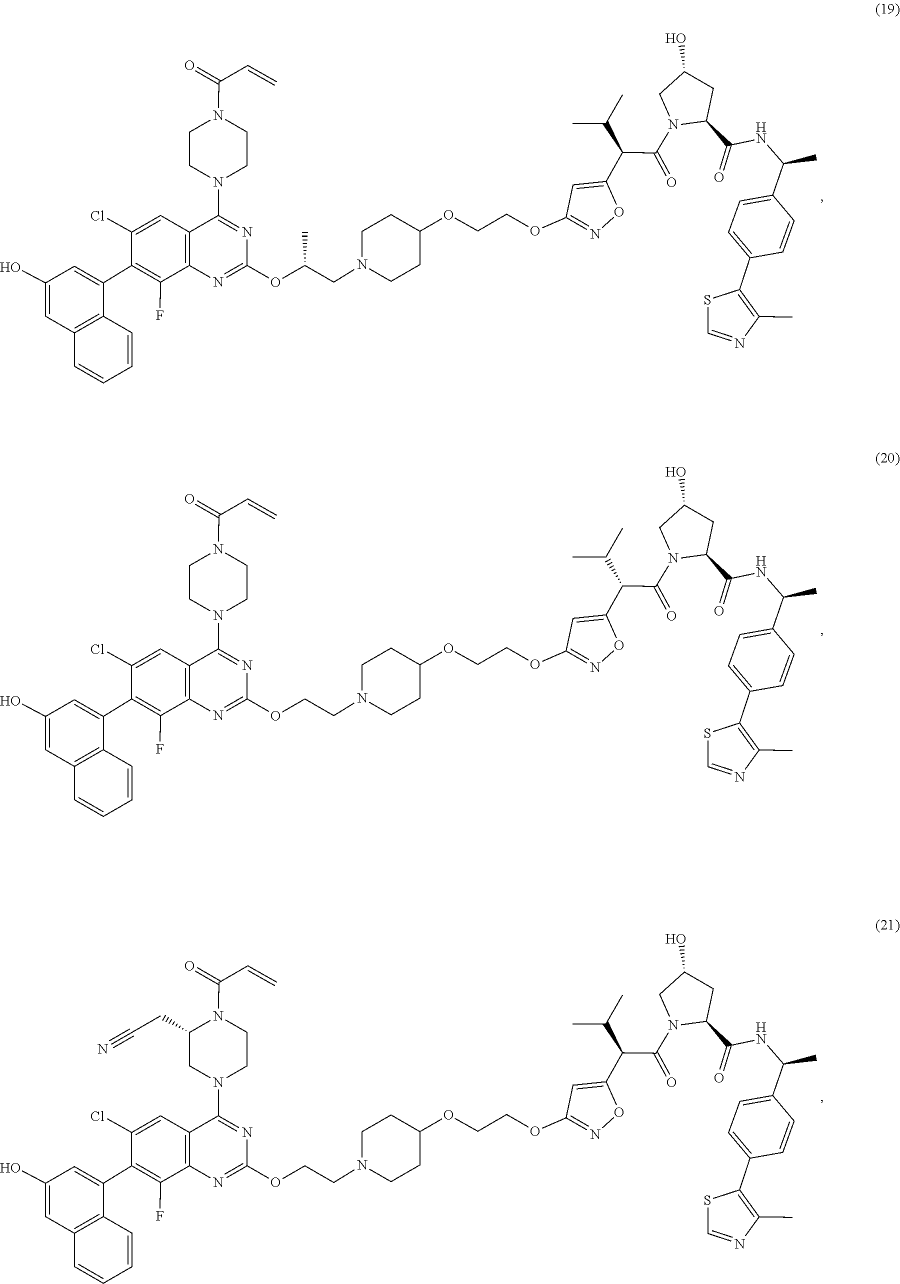 [SMILES: C=CC(=O)N1CCN(c2nc(O[C@H](C)CN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1 ; C=CC(=O)N1CCN(c2nc(OCCN3CCC(OCCOc4cc([C@@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)CC1 ; C=CC(=O)N1CCN(c2nc(OCCN3CCC(OCCOc4cc([C@H](C(=O)N5C[C@H](O)C[C@H]5C(=O)N[C@@H](C)c5ccc(-c6scnc6C)cc5)C(C)C)on4)CC3)nc3c(F)c(-c4cc(O)cc5ccccc45)c(Cl)cc23)C[C@@H]1CC#N]

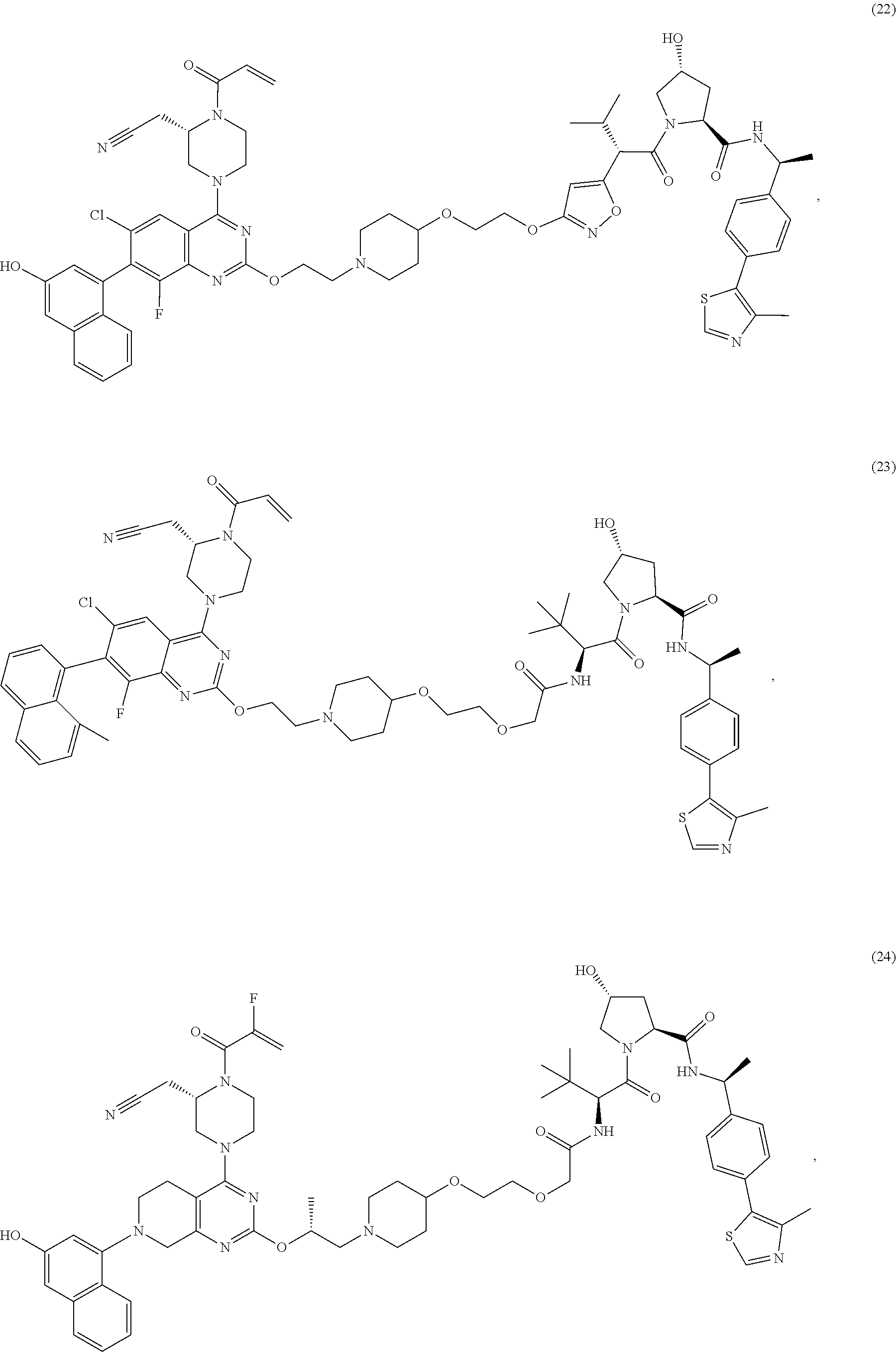

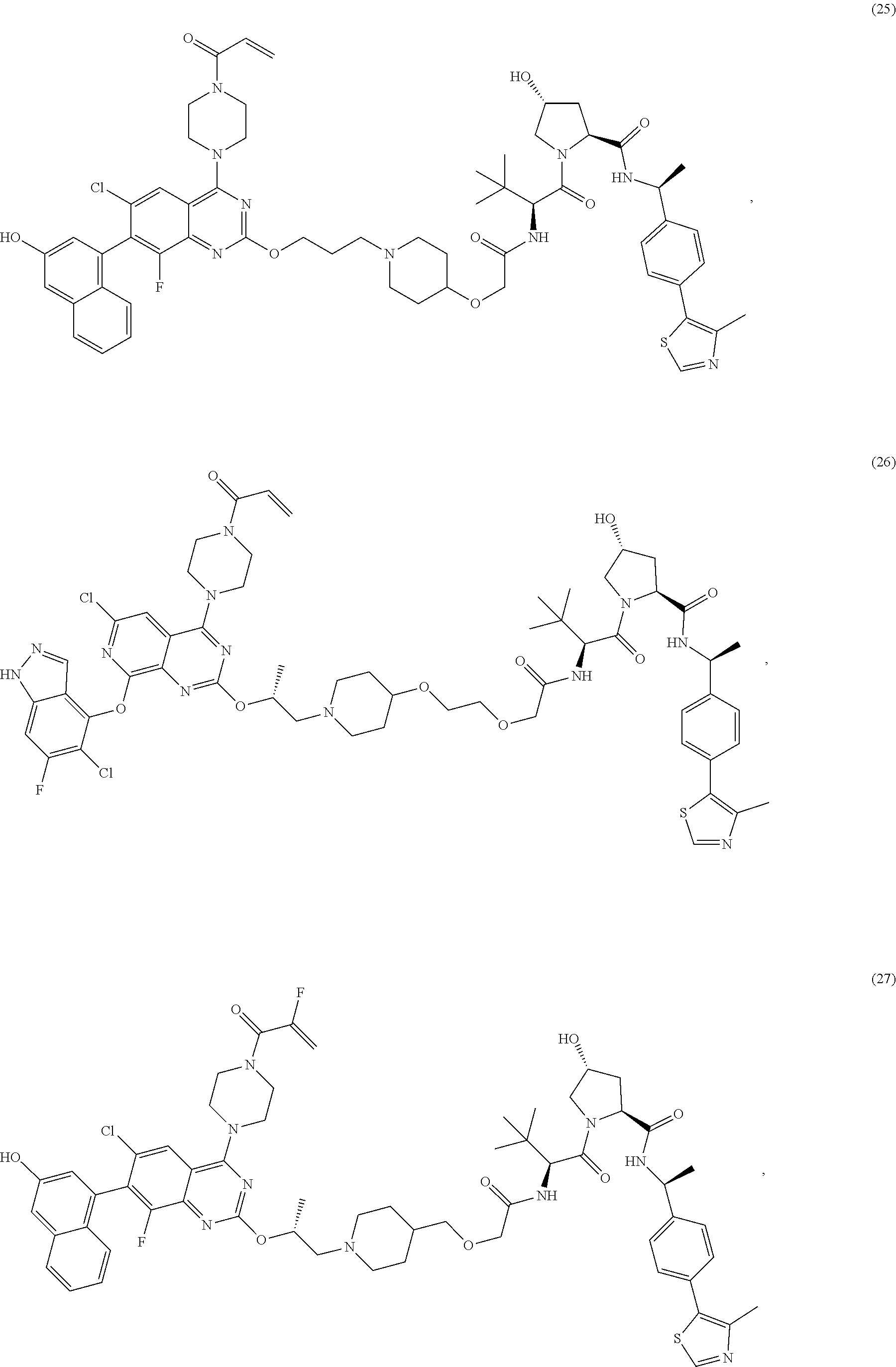 [SMILES: C=CC(=O)N1CCN(c2nc(OCCCN3CCC(OCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(Oc4c(Cl)c(F)cc5[nH]ncc45)nc(Cl)cc23)CC1 ; C=C(F)C(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

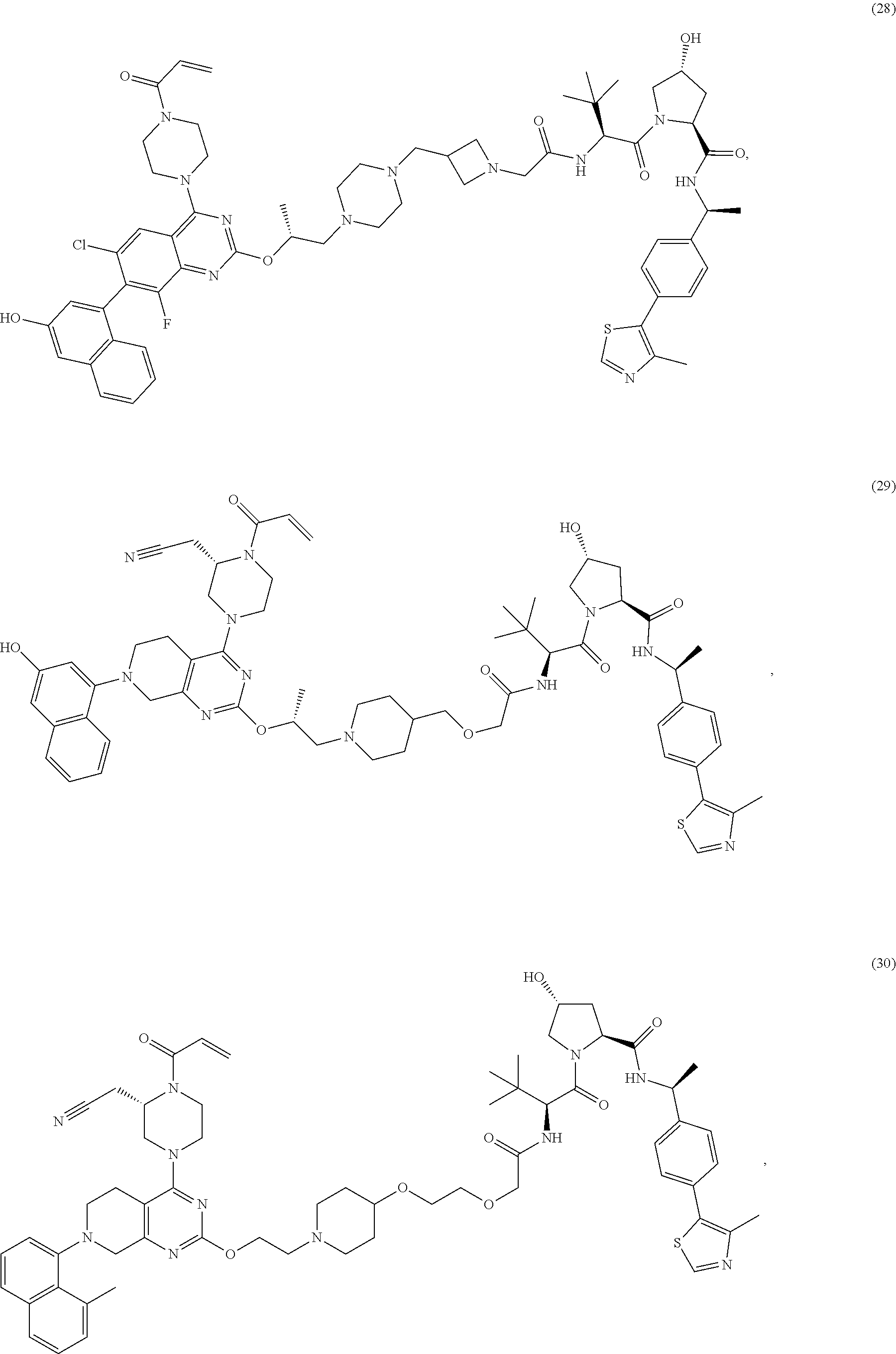 [SMILES: C=CC(=O)N1CCN(c2nc(O[C@H](C)CN3CCN(CC4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1 ; C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cc(O)cc4ccccc24)C3)C[C@@H]1CC#N ; C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cccc4cccc(C)c24)C3)C[C@@H]1CC#N]

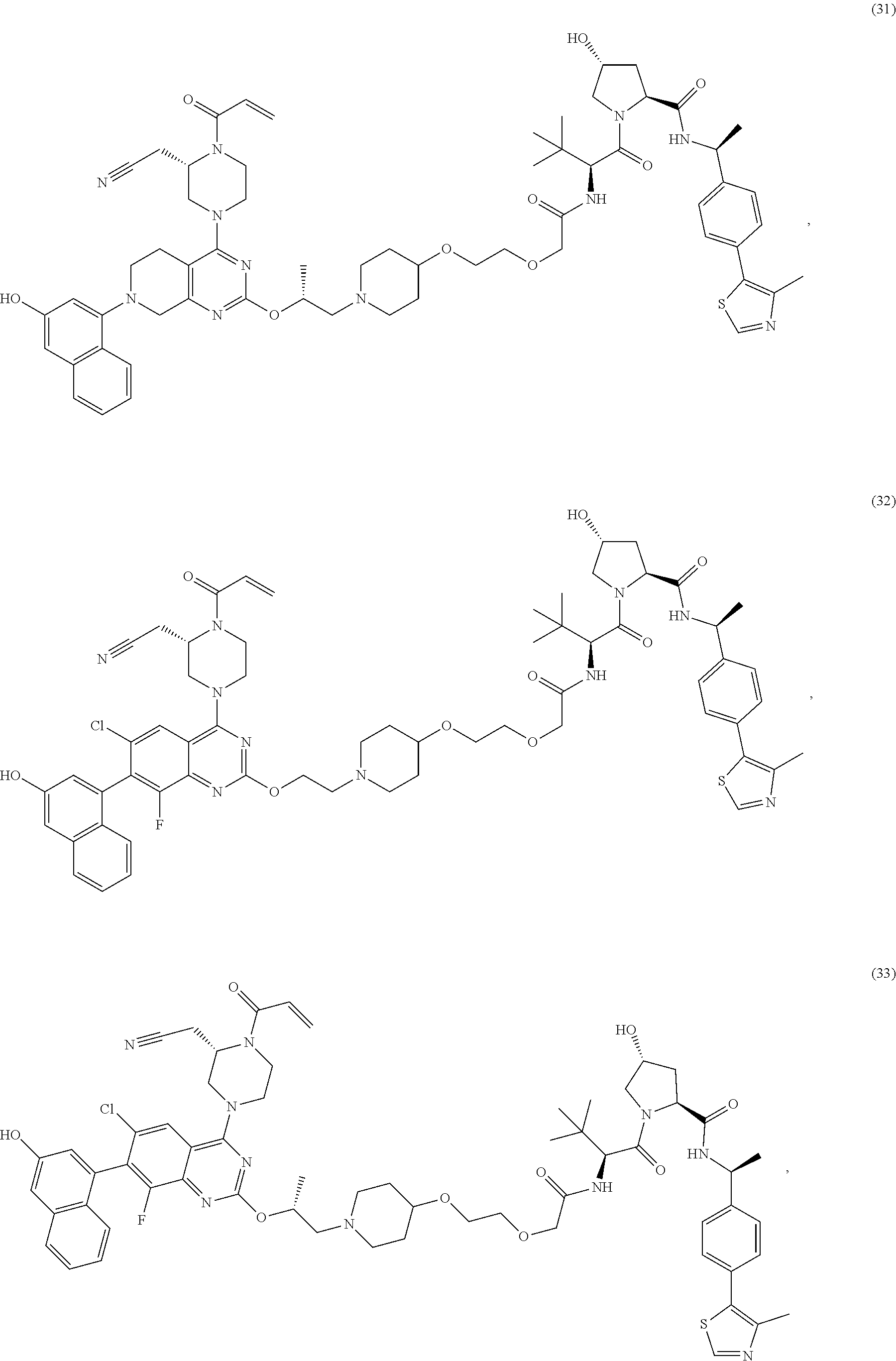

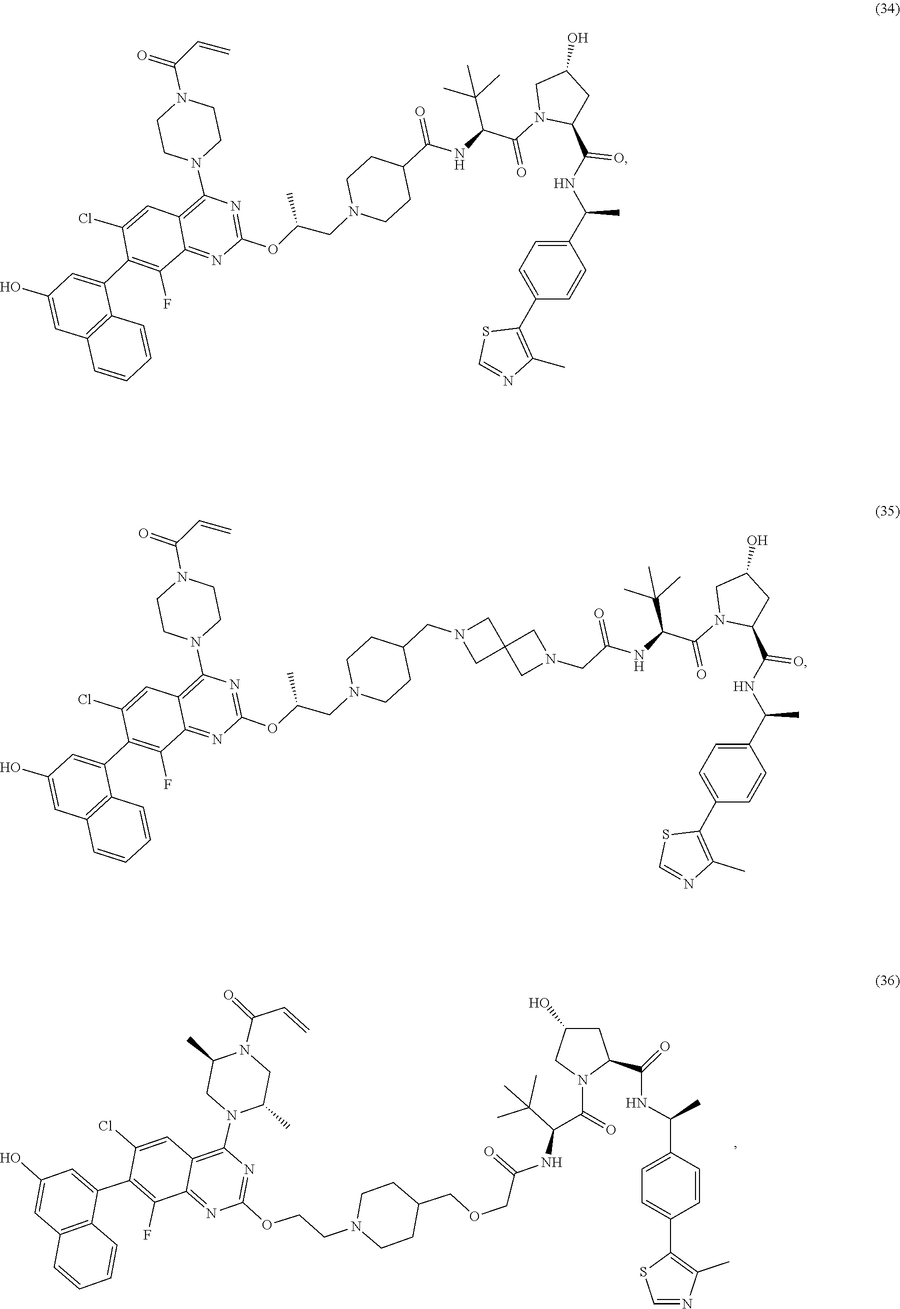

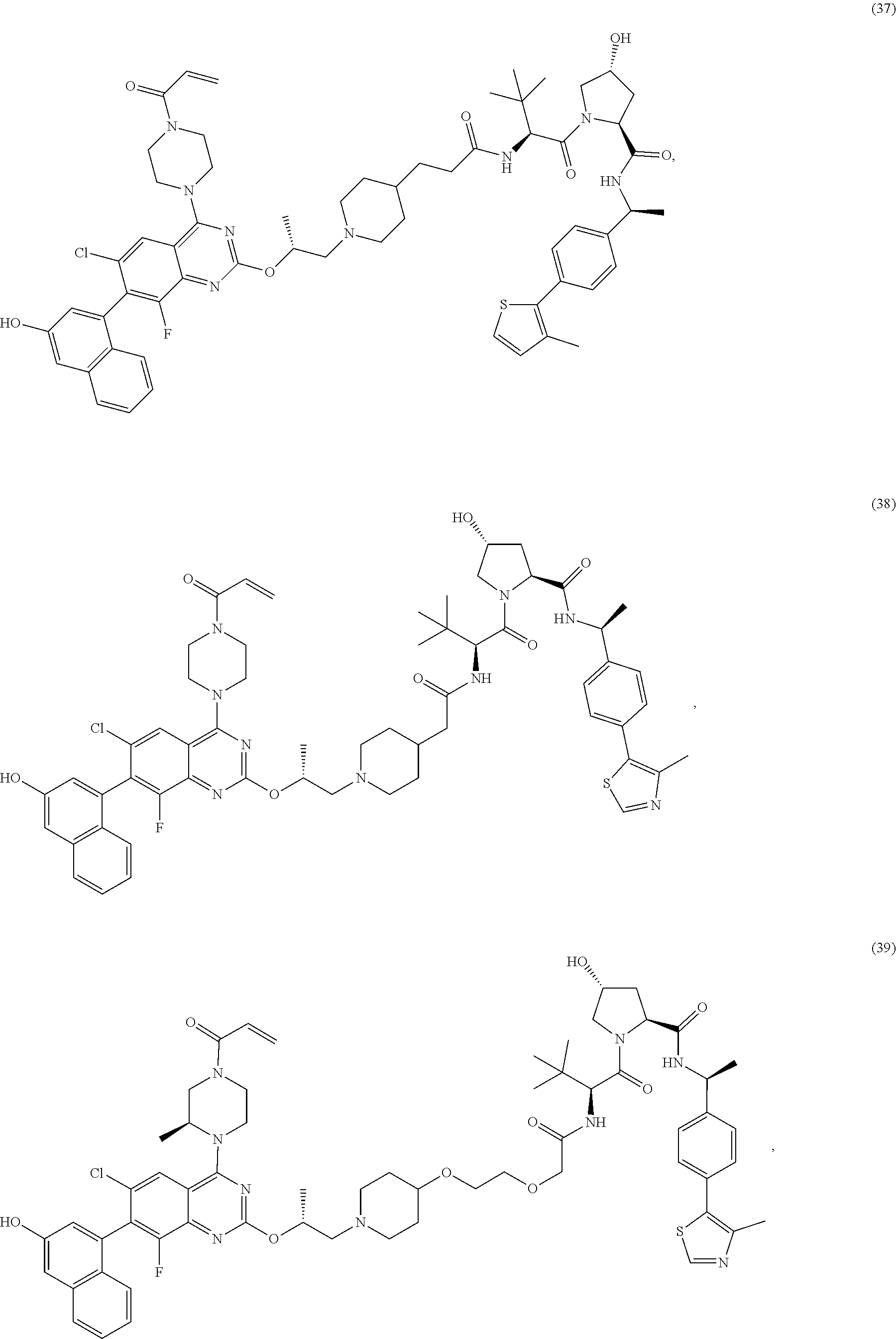 [SMILES: C=CC(=O)N1CCN(c2nc(O[C@H](C)CN3CCC(CCC(=O)N[C@H](C(=O)N4C[C@H](O)C[C@H]4C(=O)N[C@@H](C)c4ccc(-c5sccc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(CC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)[C@@H](C)C1]

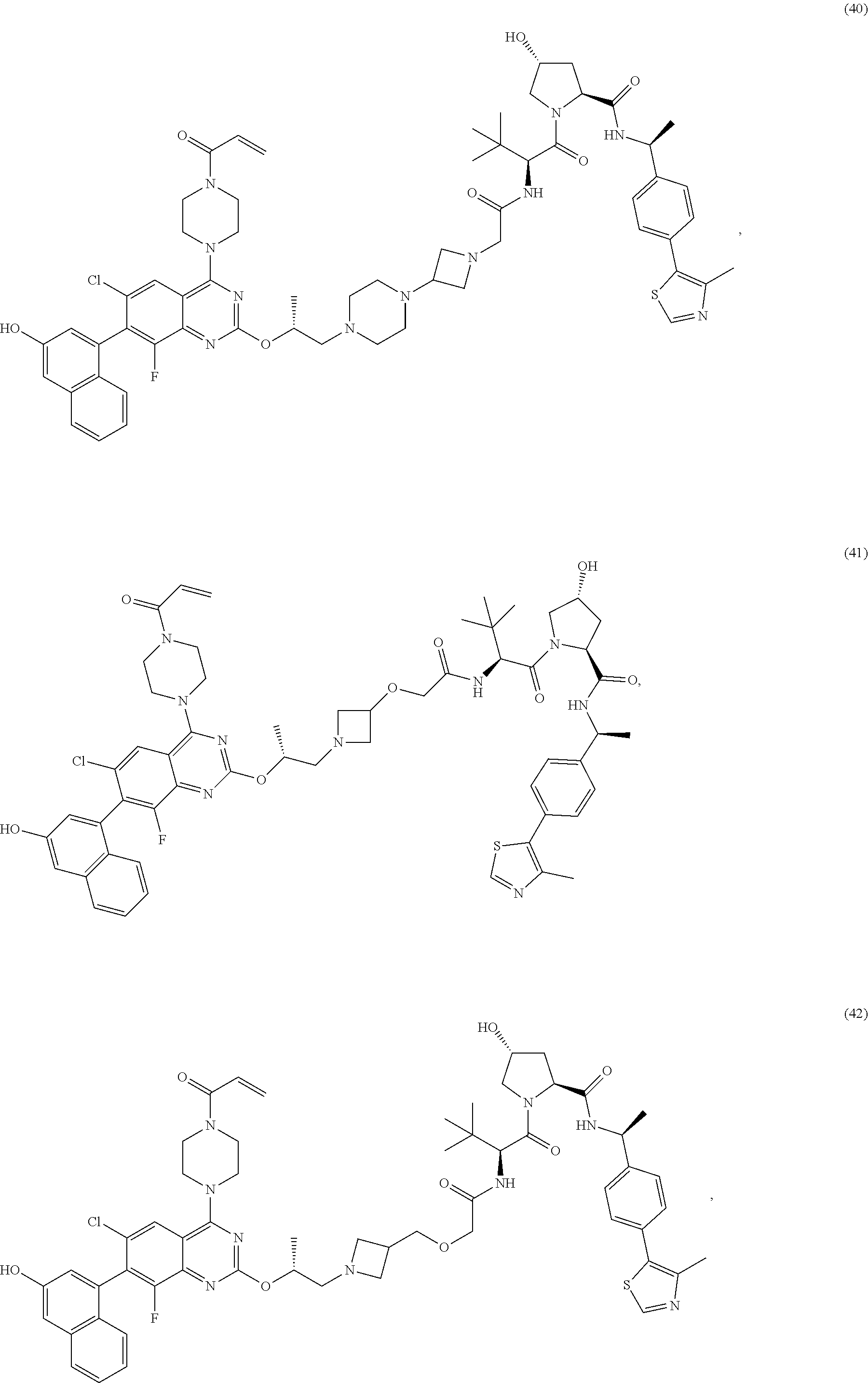 [SMILES: C=CC(=O)N1CCN(c2nc(O[C@H](C)CN3CCN(C4CN(CC(=O)N[C@H](C(=O)N5C[C@H](O)C[C@H]5C(=O)N[C@@H](C)c5ccc(-c6scnc6C)cc5)C(C)(C)C)C4)CC3)nc3c(F)c(-c4cc(O)cc5ccccc45)c(Cl)cc23)CC1 ; C=CC(=O)N1CCN(c2nc(O[C@H](C)CN3CC(OCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)CC1 ; C=CC(=O)N1CCN(c2nc(O[C@H](C)CN3CC(COCC(=O)N[C@H](C(=O)N4C[C@H](O)C[C@H]4C(=O)N[C@@H](C)c4ccc(-c5scnc5C)cc4)C(C)(C)C)C3)nc3c(F)c(-c4cc(O)cc5ccccc45)c(Cl)cc23)CC1]

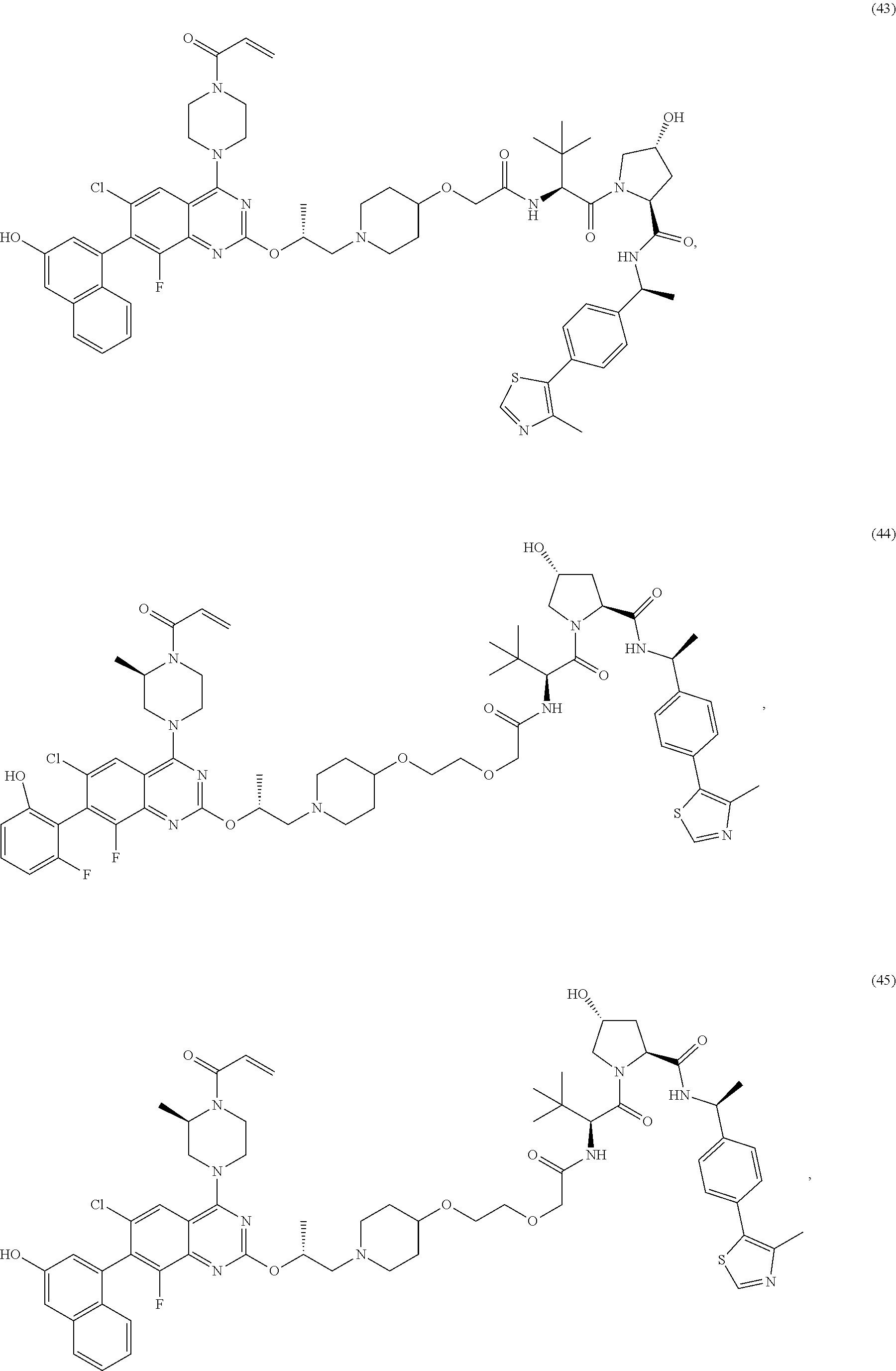 [SMILES: C=CC(=O)N1CCN(c2nc(O[C@H](C)CN3CCC(OCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(O)cccc4F)c(Cl)cc23)C[C@H]1C ; C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)C[C@H]1C]

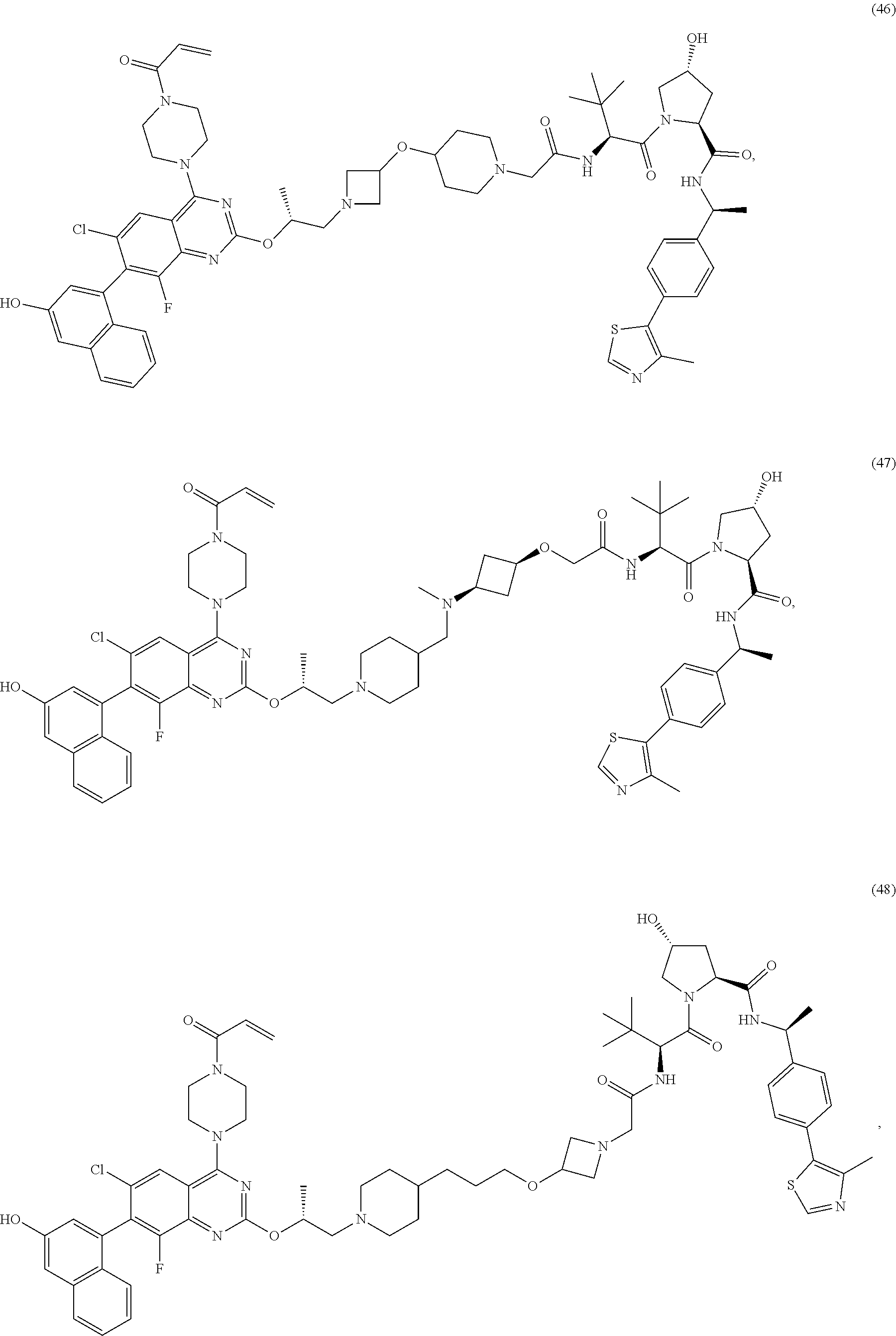

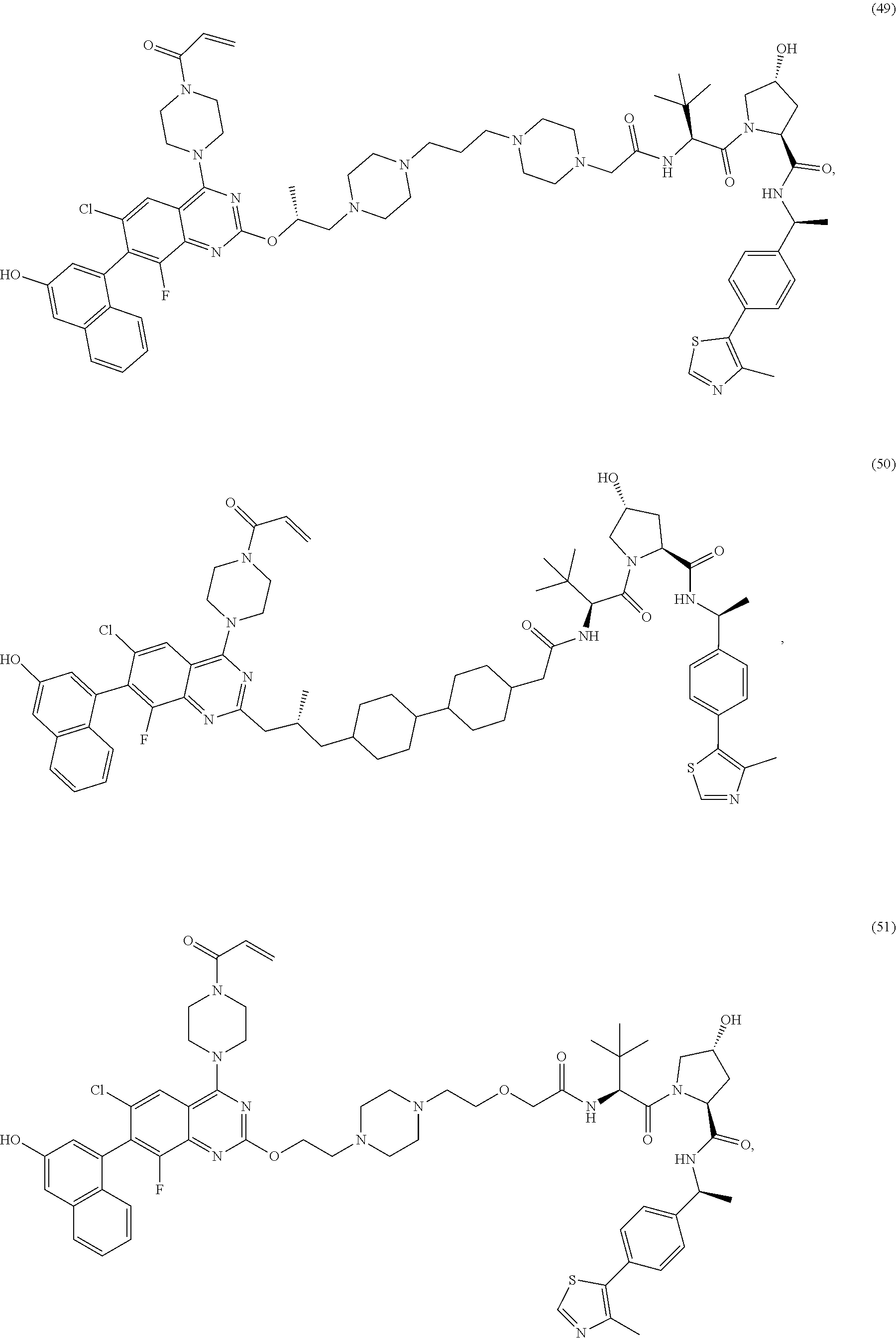

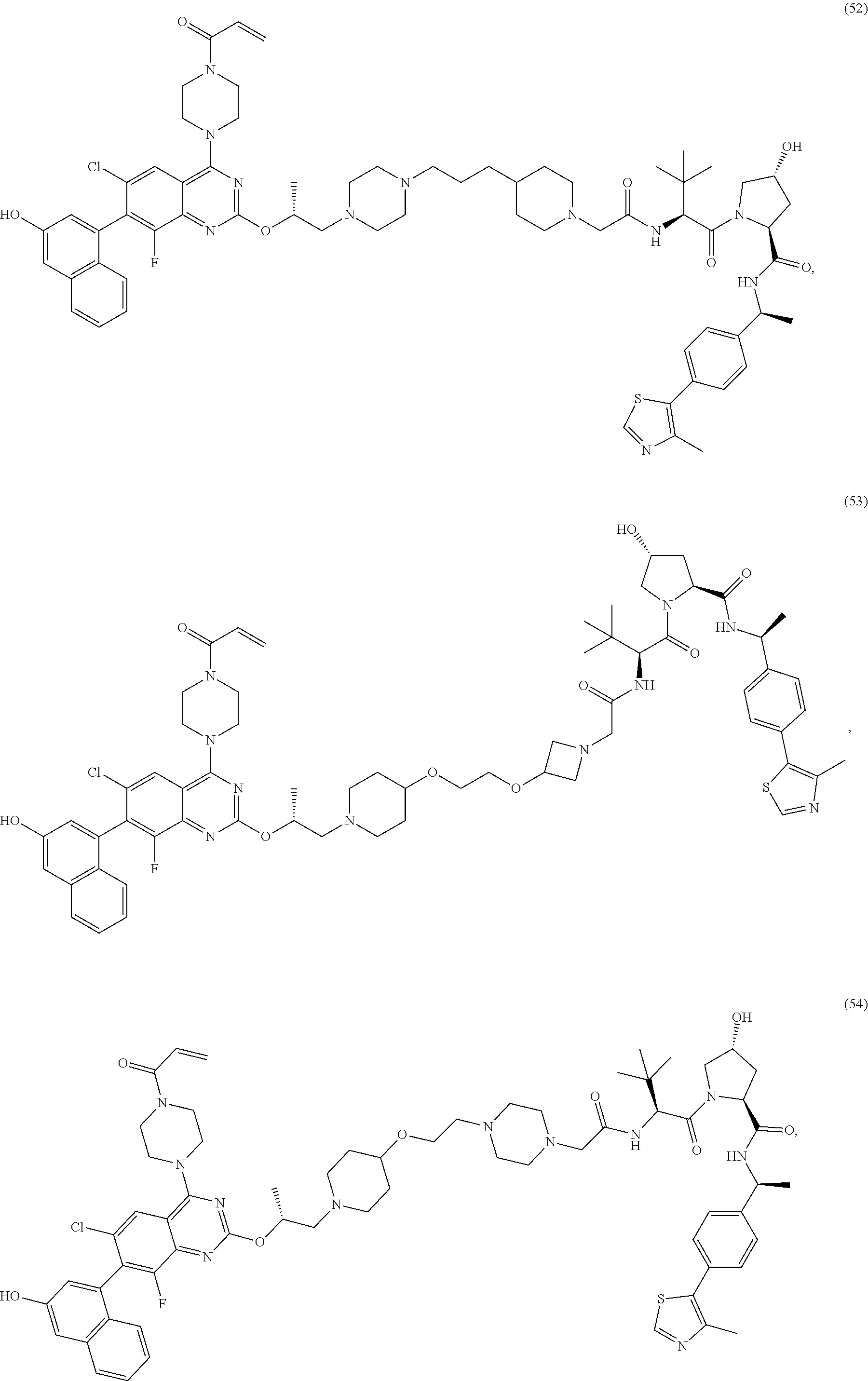

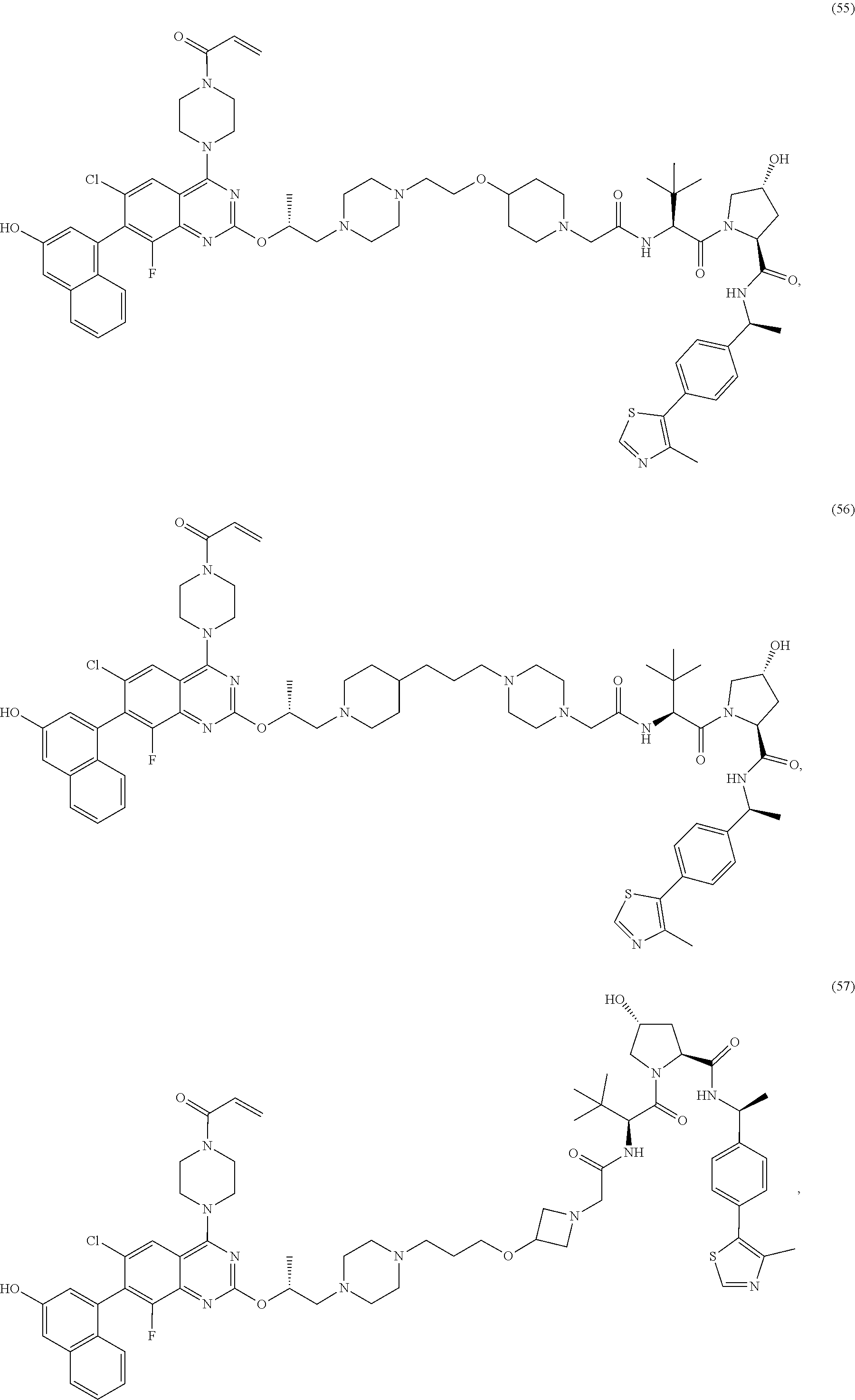

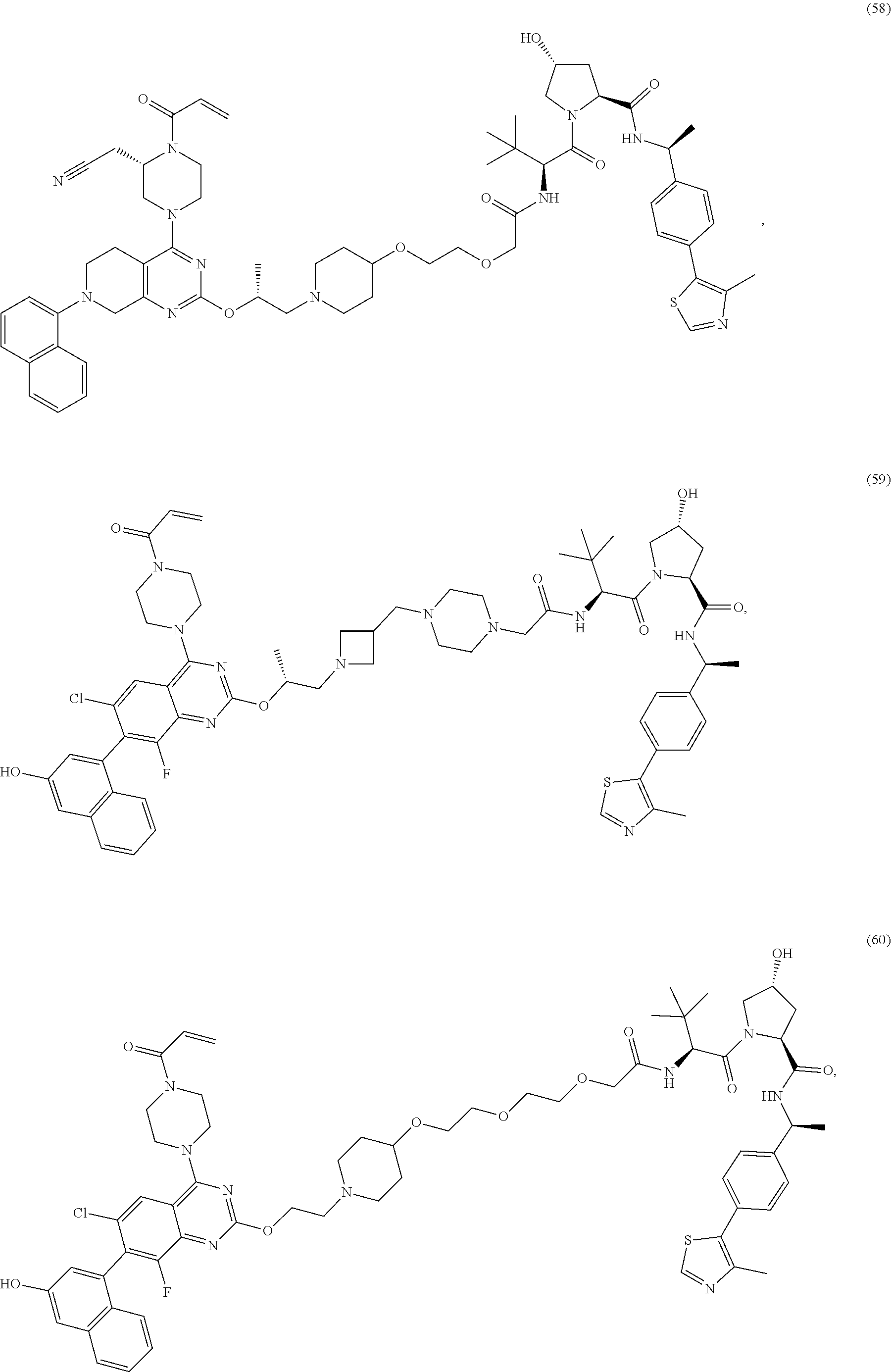 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c2CCN(c2cccc4ccccc24)C3)C[C@@H]1CC#N ; C=CC(=O)N1CCN(c2nc(O[C@H](C)CN3CC(CN4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1 ; C=CC(=O)N1CCN(c2nc(OCCN3CCC(OCCOCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

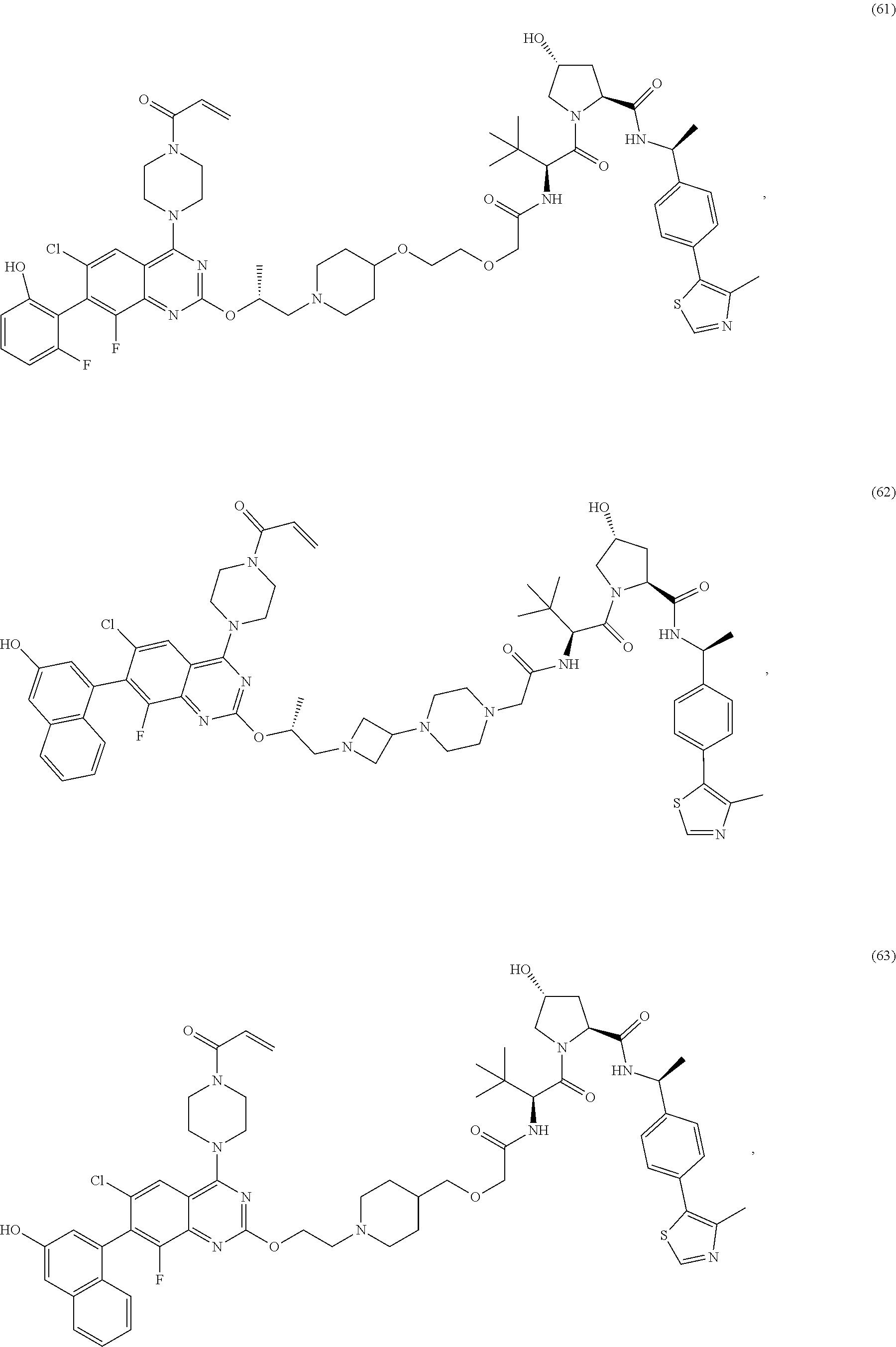 [SMILES: C=CC(=O)N1CCN(c2nc(O[C@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4c(O)cccc4F)c(Cl)cc23)CC1 ; C=CC(=O)N1CCN(c2nc(O[C@H](C)CN3CC(N4CCN(CC(=O)N[C@H](C(=O)N5C[C@H](O)C[C@H]5C(=O)N[C@@H](C)c5ccc(-c6scnc6C)cc5)C(C)(C)C)CC4)C3)nc3c(F)c(-c4cc(O)cc5ccccc45)c(Cl)cc23)CC1 ; C=CC(=O)N1CCN(c2nc(OCCN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

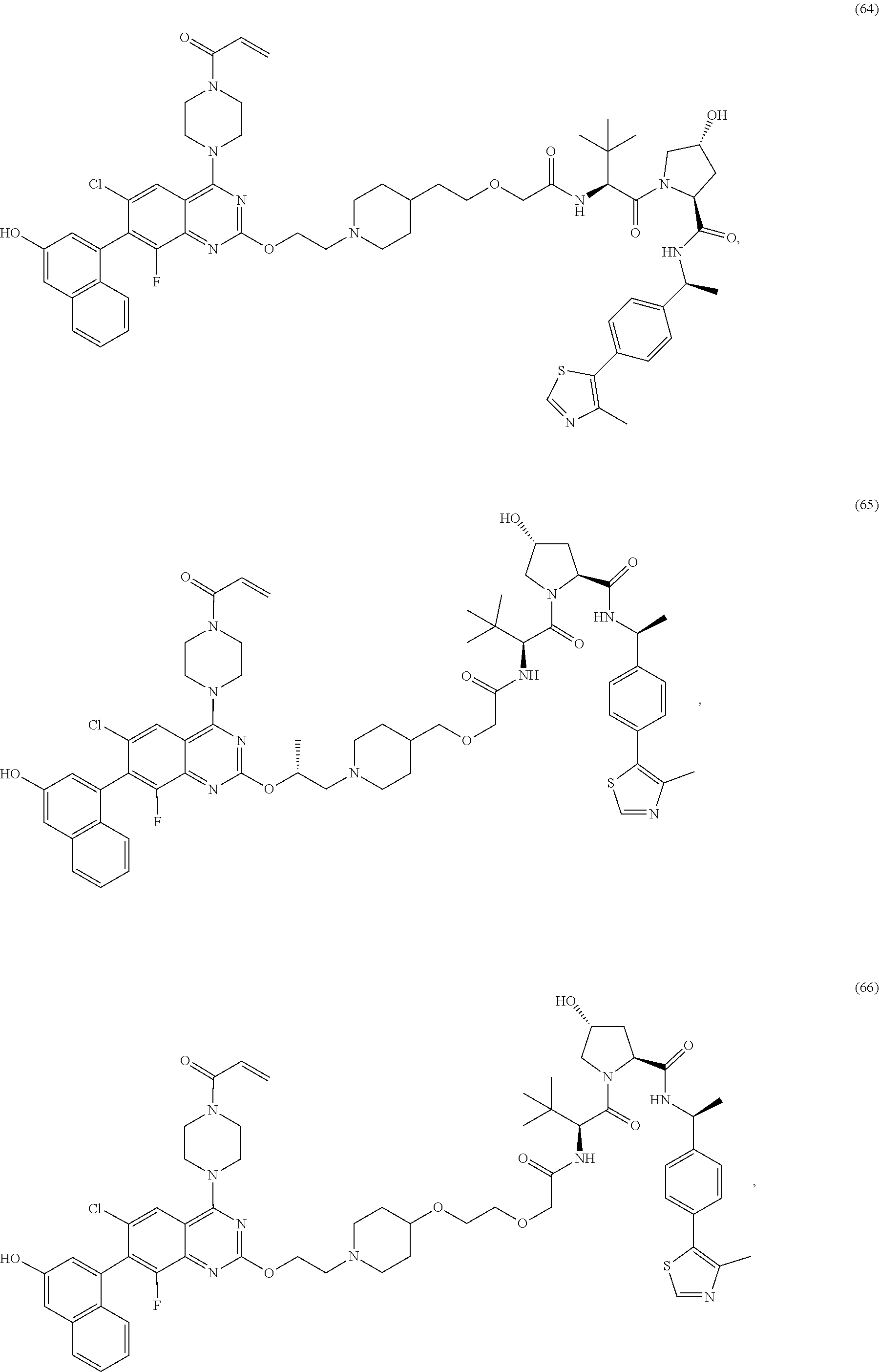 [SMILES: C=CC(=O)N1CCN(c2nc(OCCN3CCC(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(COCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(OCCN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1]

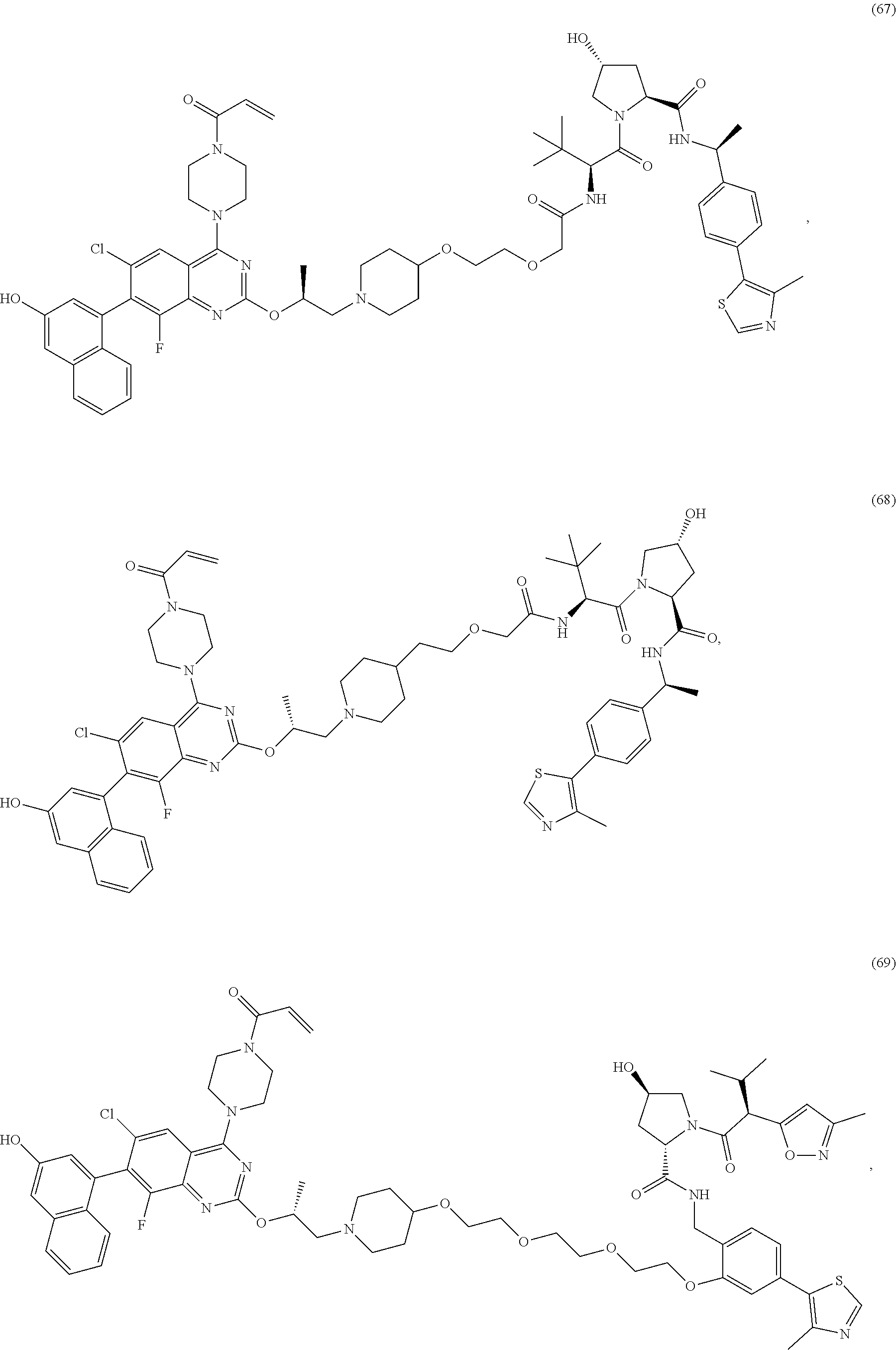 [SMILES: C=CC(=O)N1CCN(c2nc(O[C@@H](C)CN3CCC(OCCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(CCOCC(=O)N[C@H](C(=O)N4C[C@H](O)C[C@H]4C(=O)N[C@@H](C)c4ccc(-c5scnc5C)cc4)C(C)(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

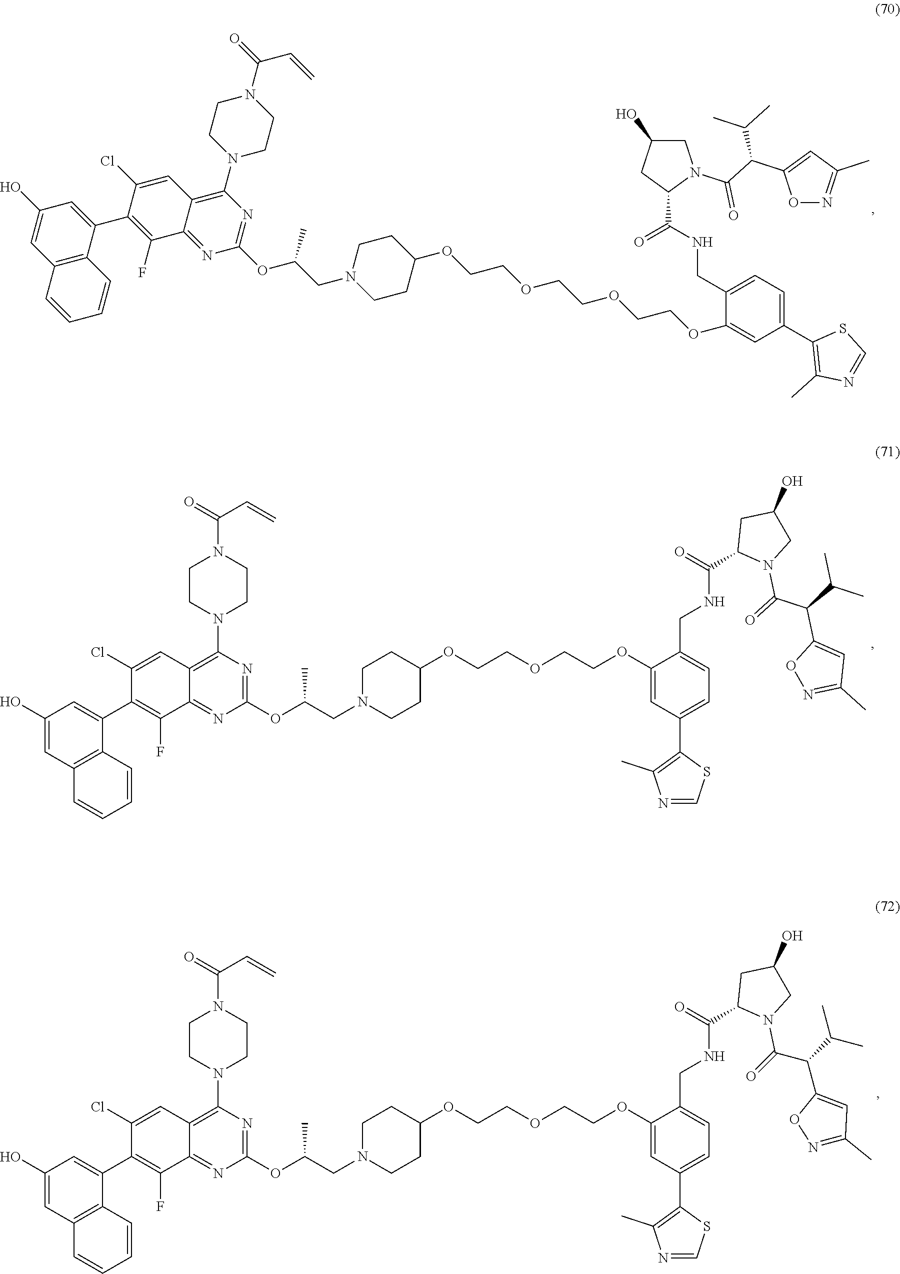 [SMILES: C=CC(=O)N1CCN(c2nc(O[C@H](C)CN3CCC(OCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1]

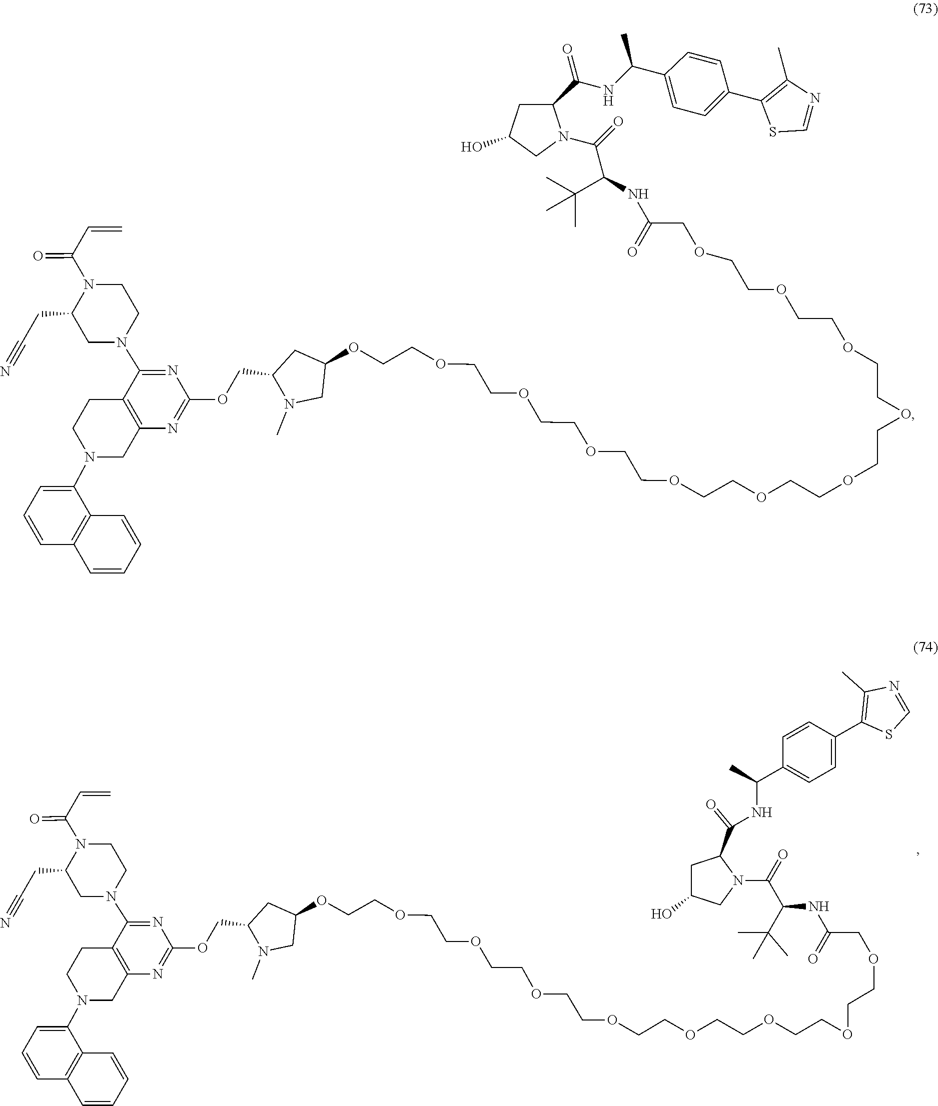 [SMILES: C=CC(=O)N1CCN(c2nc(OC[C@@H]3C[C@@H](OCCOCCOCCOCCOCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CN3C)nc3c2CCN(c2cccc4ccccc24)C3)C[C@@H]1CC#N ; C=CC(=O)N1CCN(c2nc(OC[C@@H]3C[C@@H](OCCOCCOCCOCCOCCOCCOCCOCCOCC(=O)N[C@H](C(=O)N4C[C@H](O)C[C@H]4C(=O)N[C@@H](C)c4ccc(-c5scnc5C)cc4)C(C)(C)C)CN3C)nc3c2CCN(c2cccc4ccccc24)C3)C[C@@H]1CC#N]

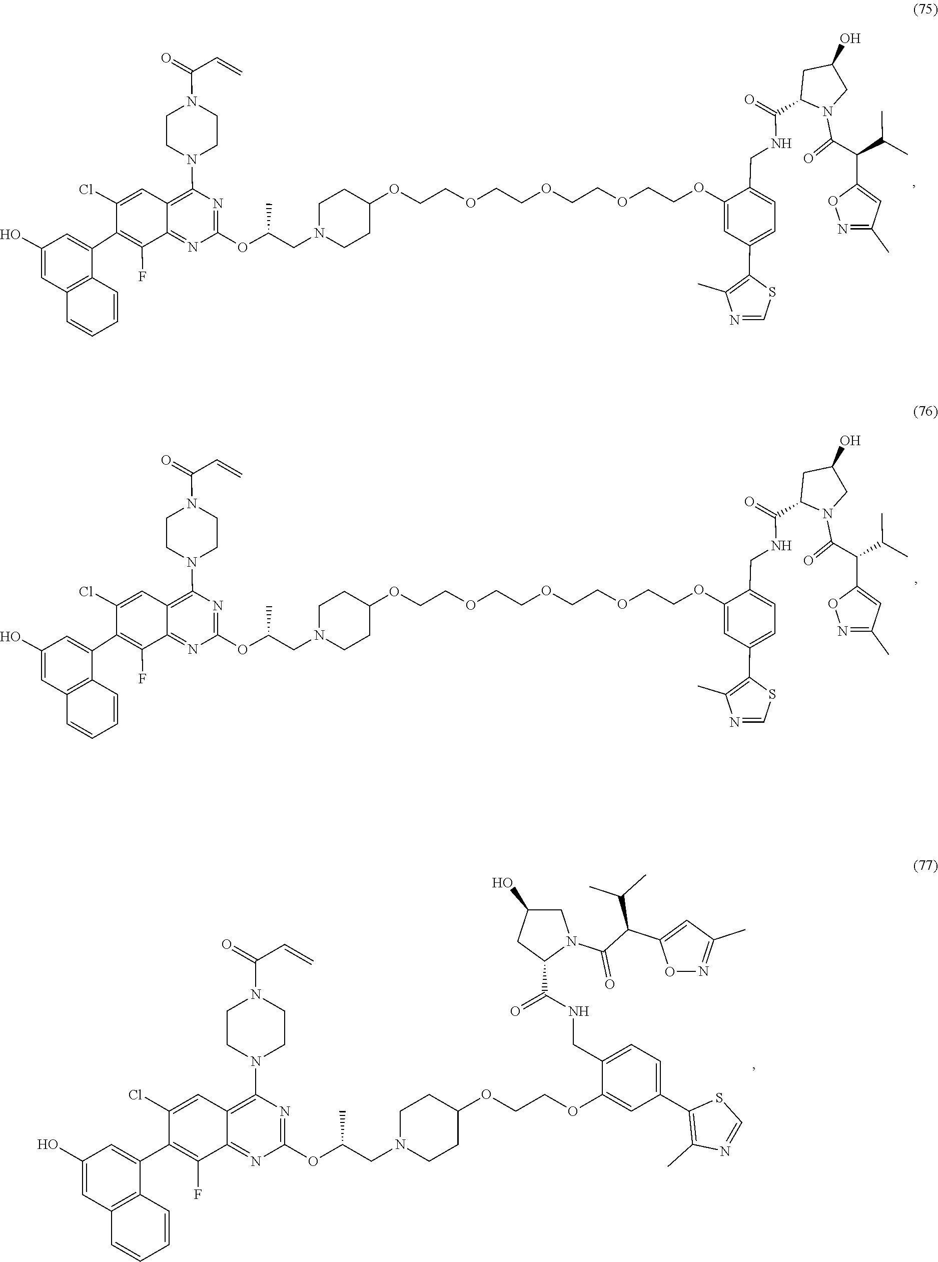 [SMILES: C=CC(=O)N1CCN(c2nc(O[C@H](C)CN3CCC(OCCOCCOCCOCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1 ; C=CC(=O)N1CCN(c2nc(O[C@H](C)CN3CCC(OCCOCCOCCOCCOc4cc(-c5scnc5C)ccc4CNC(=O)[C@@H]4C[C@@H](O)CN4C(=O)[C@@H](c4cc(C)no4)C(C)C)CC3)nc3c(F)c(-c4cc(O)cc5ccccc45)c(Cl)cc23)CC1 ; C=CC(=O)N1CCN(c2nc(O[C@H](C)CN3CCC(OCCOc4cc(-c5scnc5C)ccc4CNC(=O)[C@@H]4C[C@@H](O)CN4C(=O)[C@H](c4cc(C)no4)C(C)C)CC3)nc3c(F)c(-c4cc(O)cc5ccccc45)c(Cl)cc23)CC1]

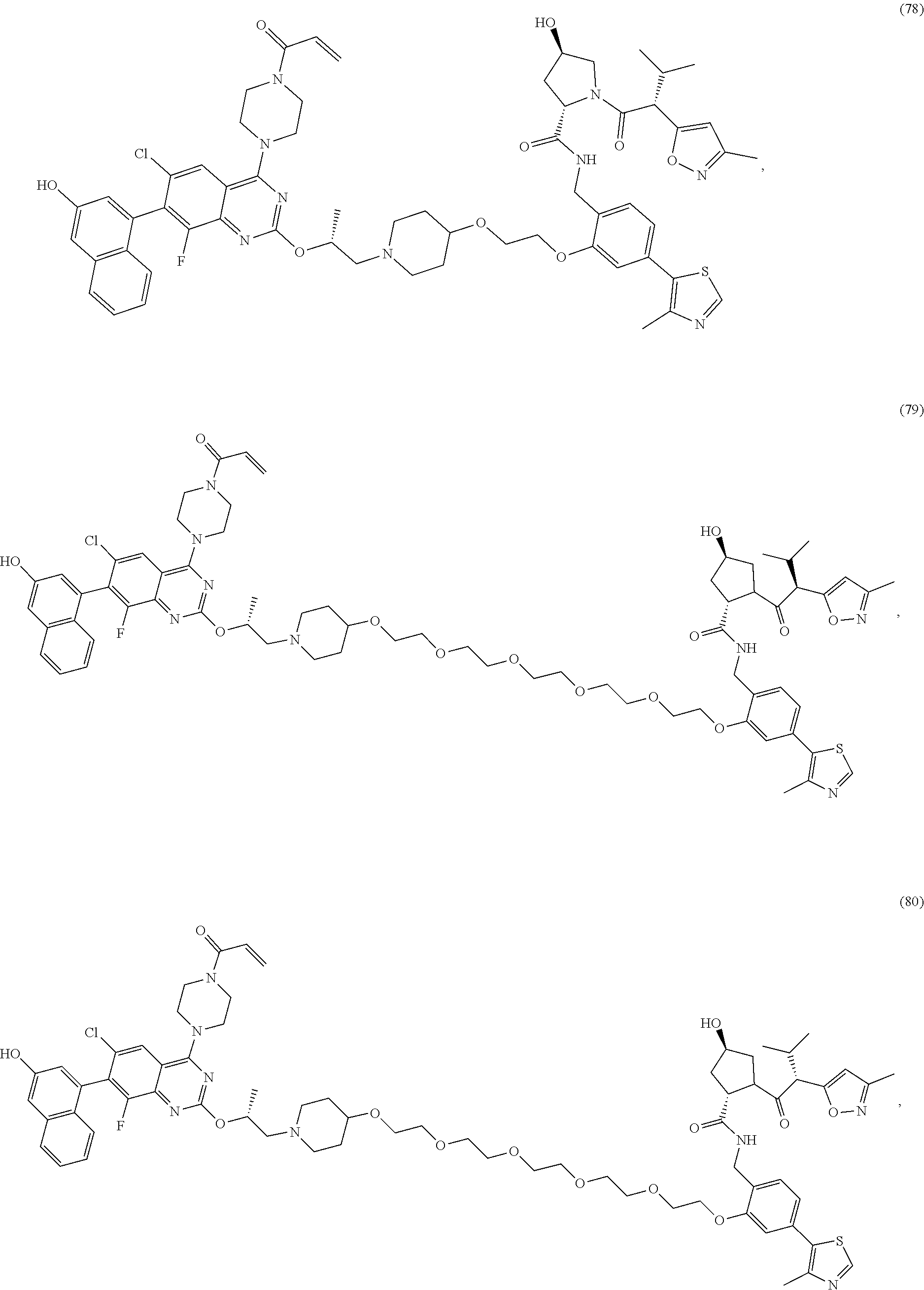

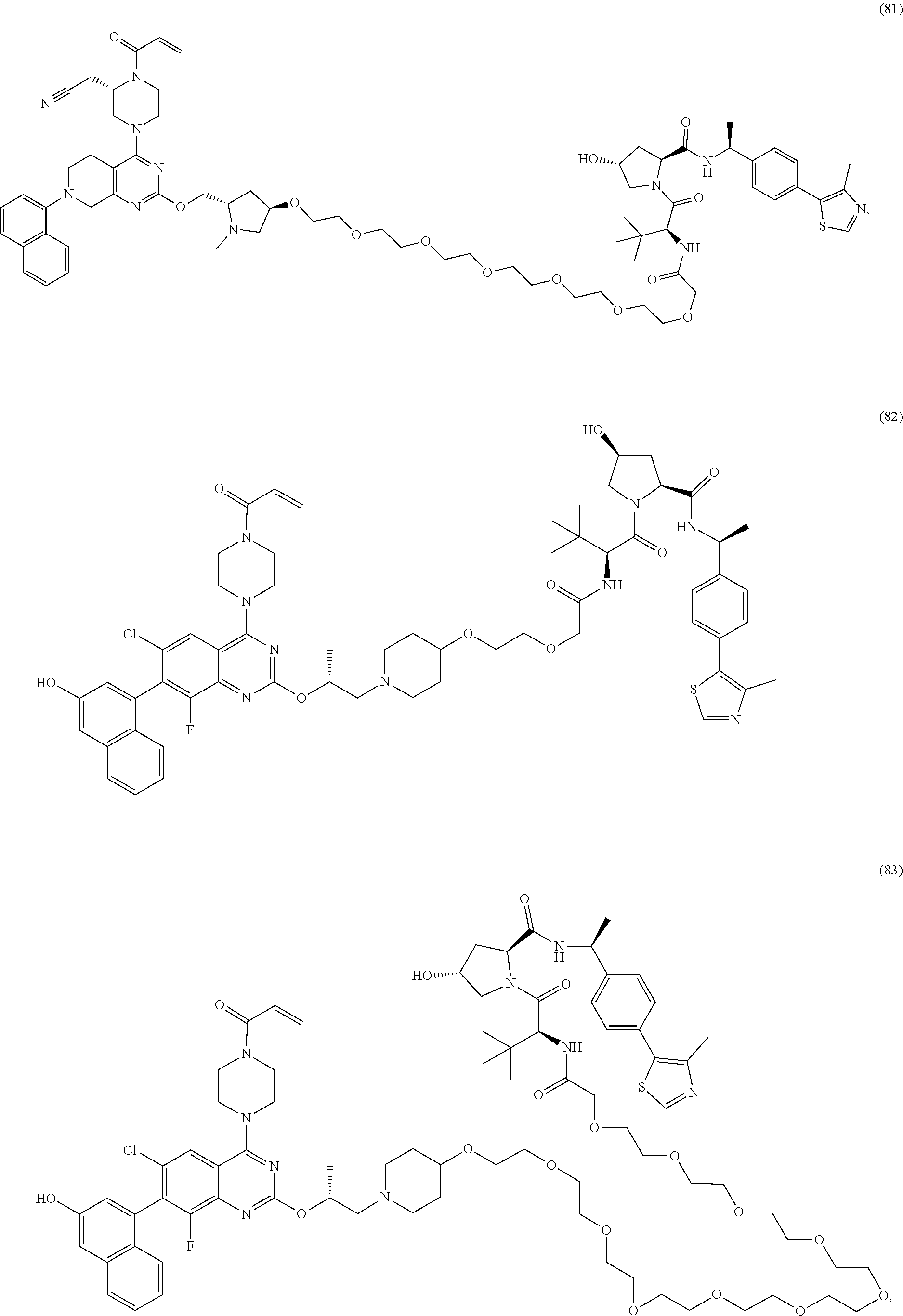

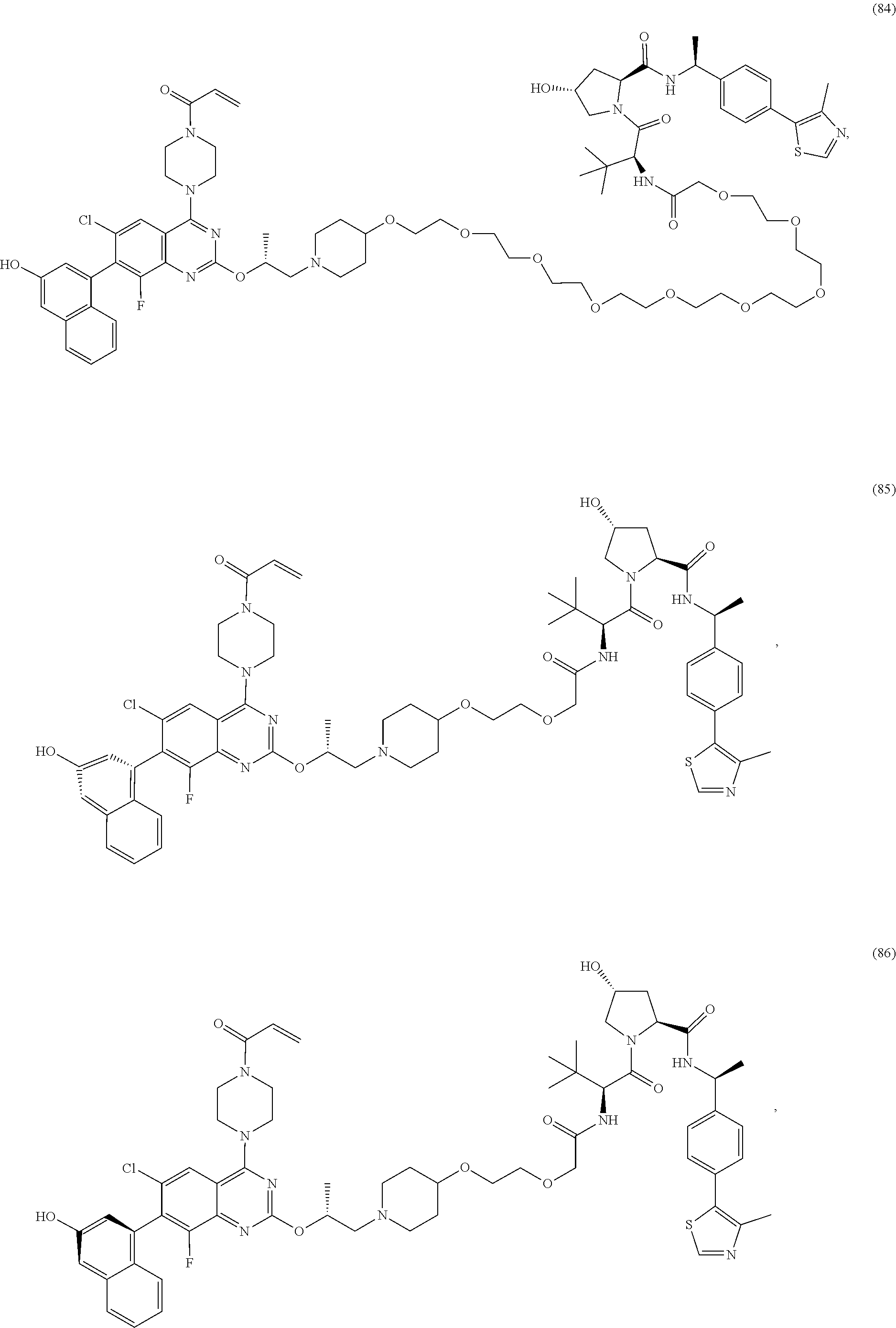

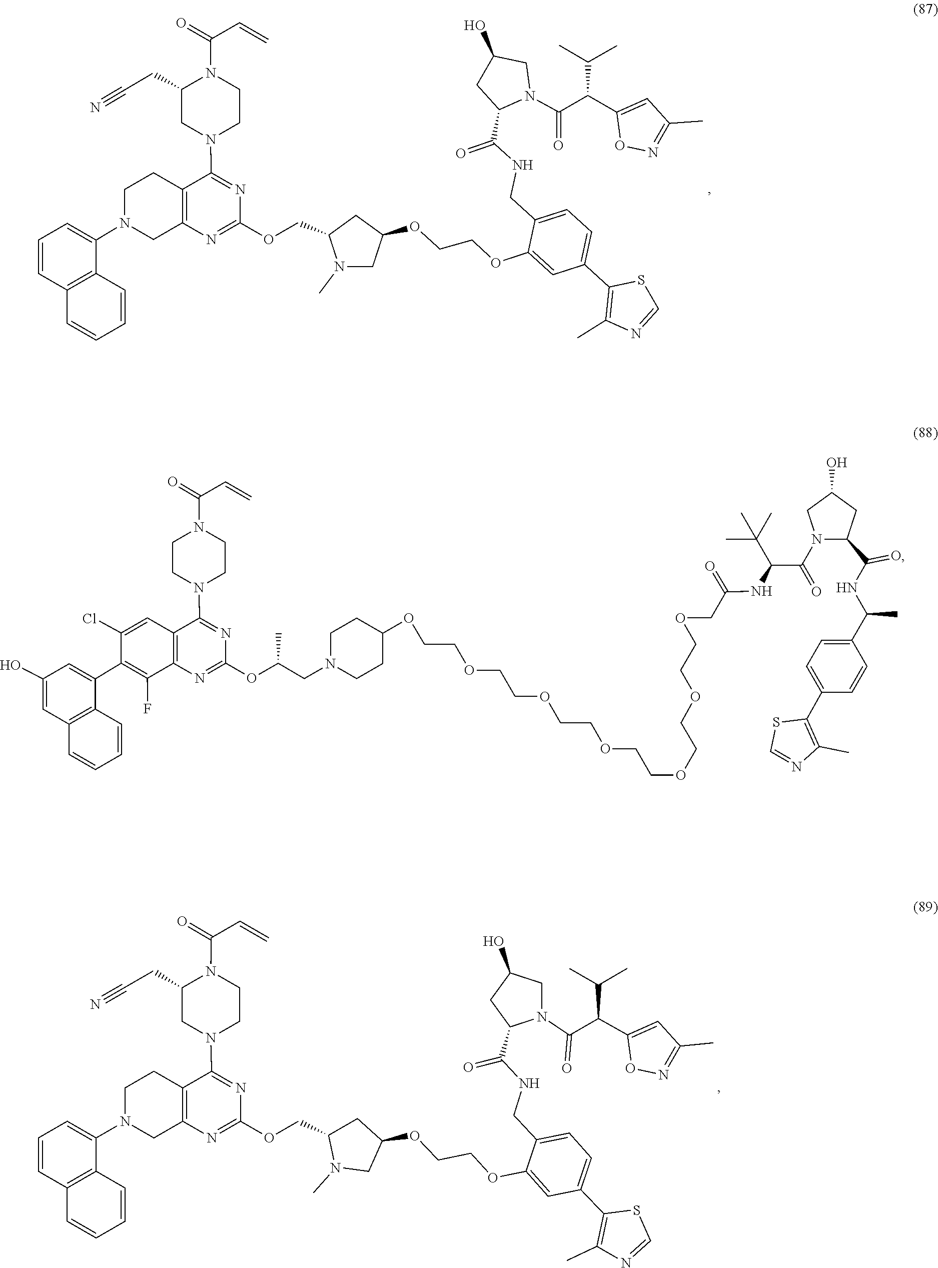

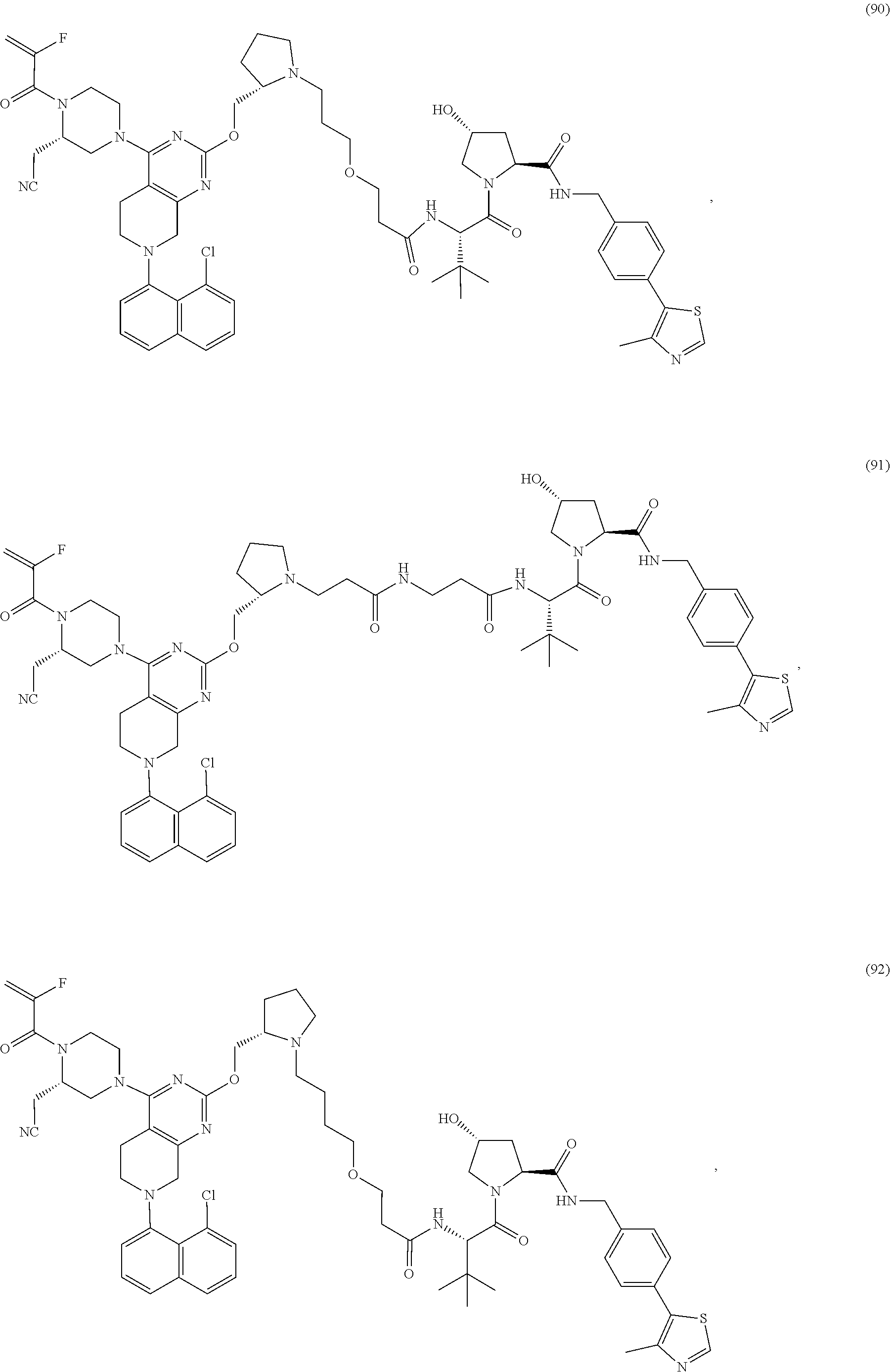 [SMILES: C=C(F)C(=O)N1CCN(c2nc(OC[C@@H]3CCCN3CCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N ; C=C(F)C(=O)N1CCN(c2nc(OC[C@@H]3CCCN3CCC(=O)NCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N ; C=C(F)C(=O)N1CCN(c2nc(OC[C@@H]3CCCN3CCCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N]

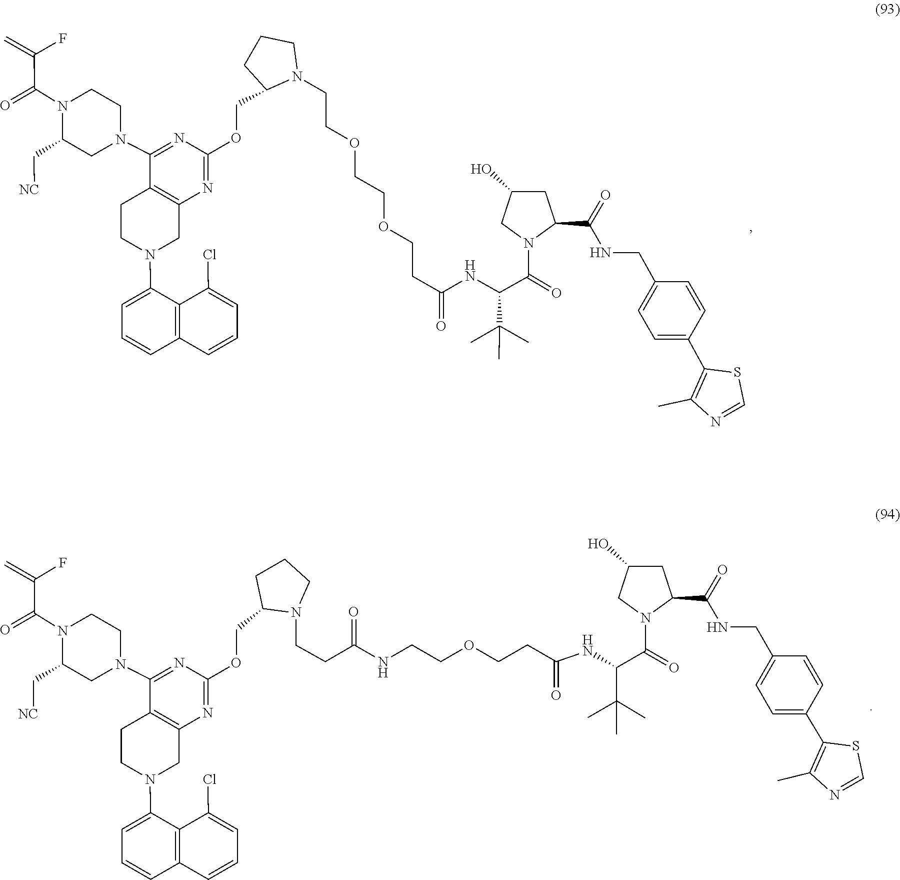 [SMILES: C=C(F)C(=O)N1CCN(c2nc(OC[C@@H]3CCCN3CCOCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N ; C=C(F)C(=O)N1CCN(c2nc(OC[C@@H]3CCCN3CCC(=O)NCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N]

21. The compound according to claim 1, wherein the compound is selected from the group consisting of compounds 95-173 of Table 7:

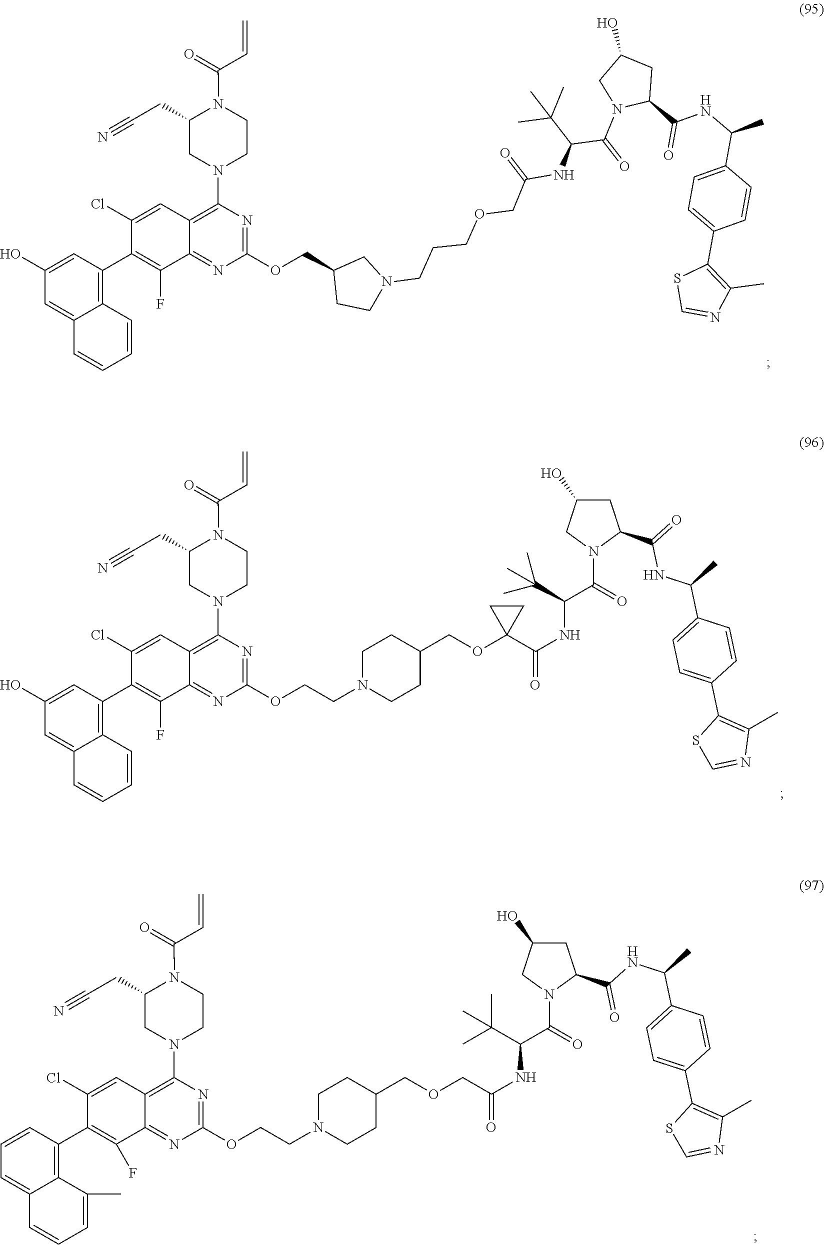

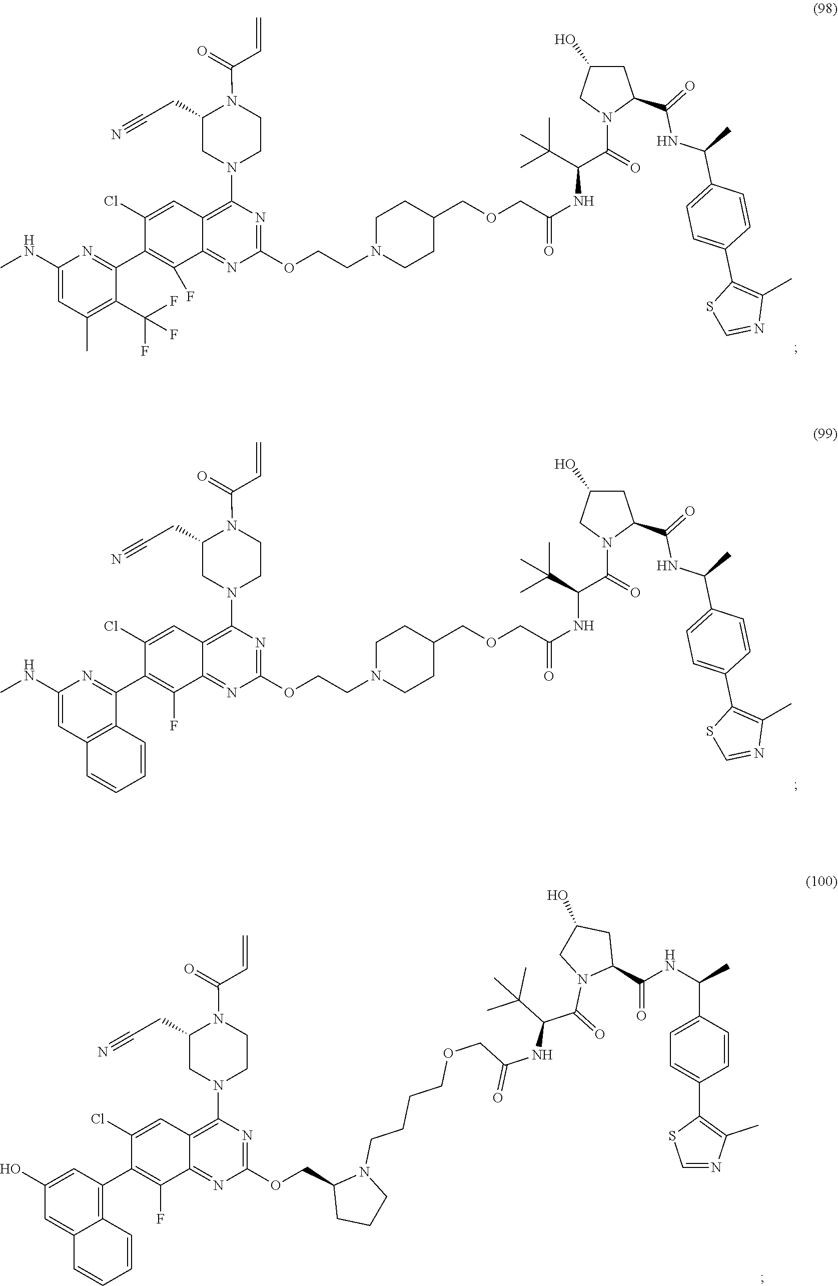

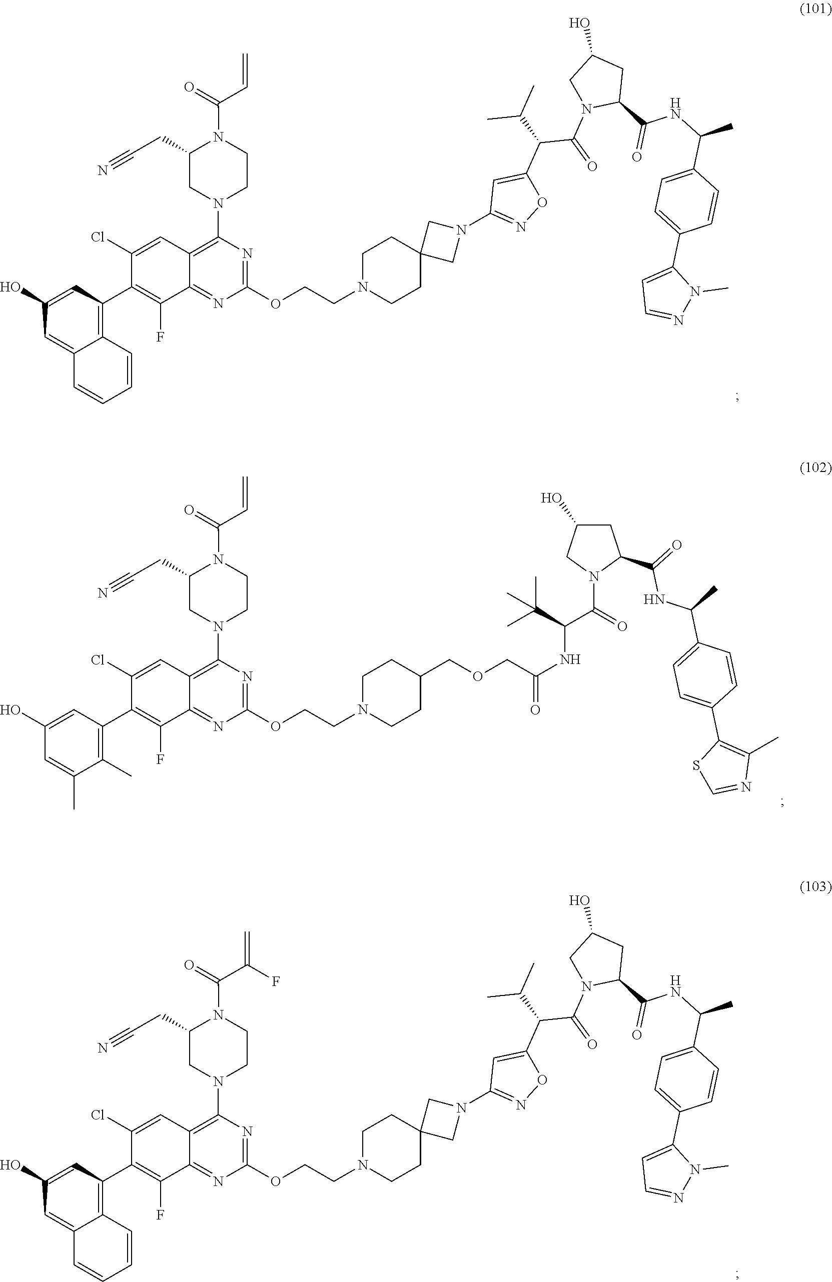

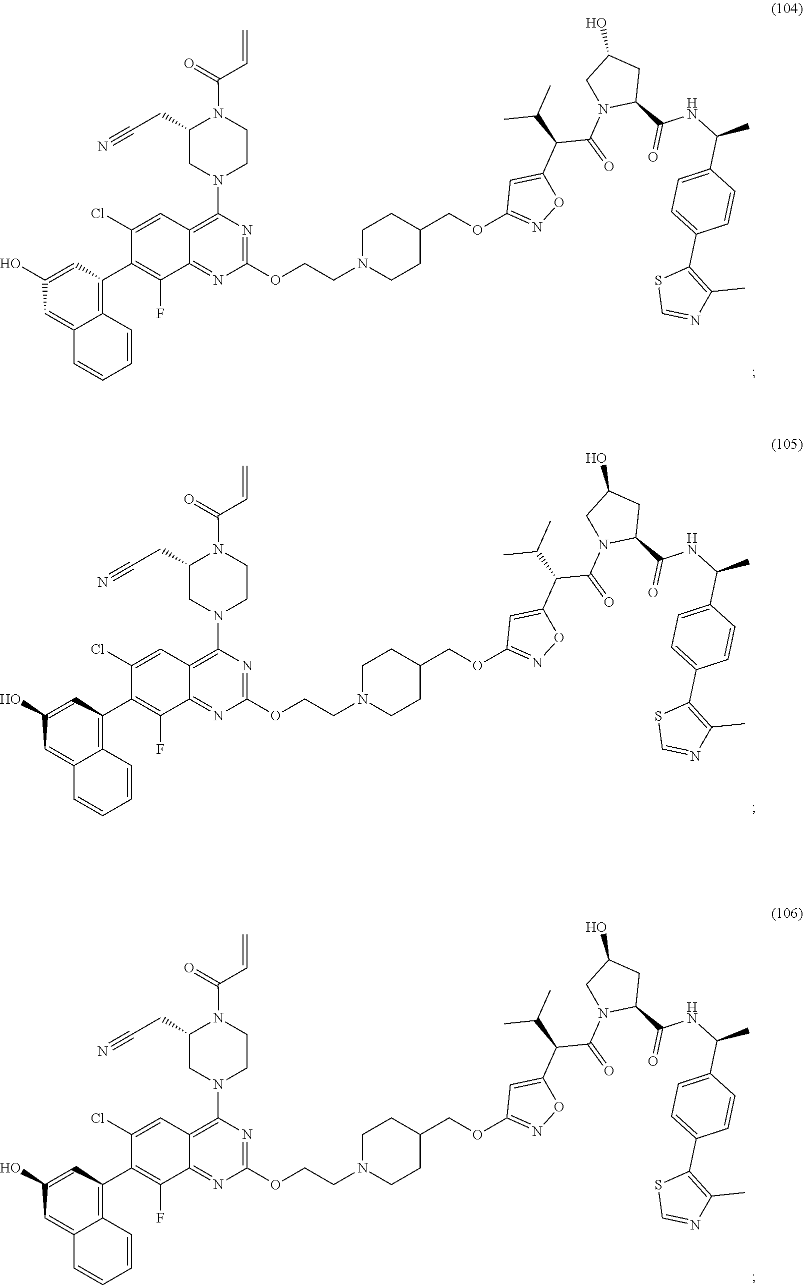

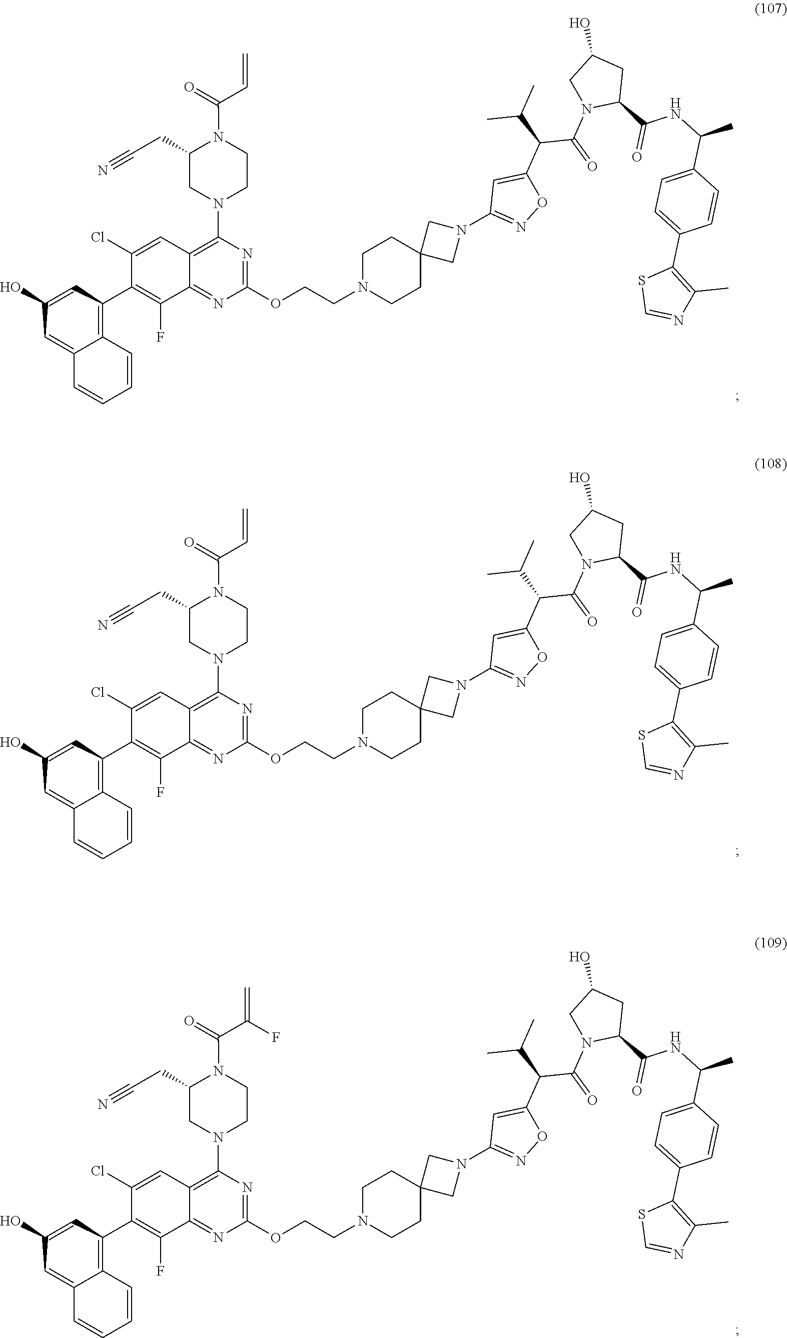

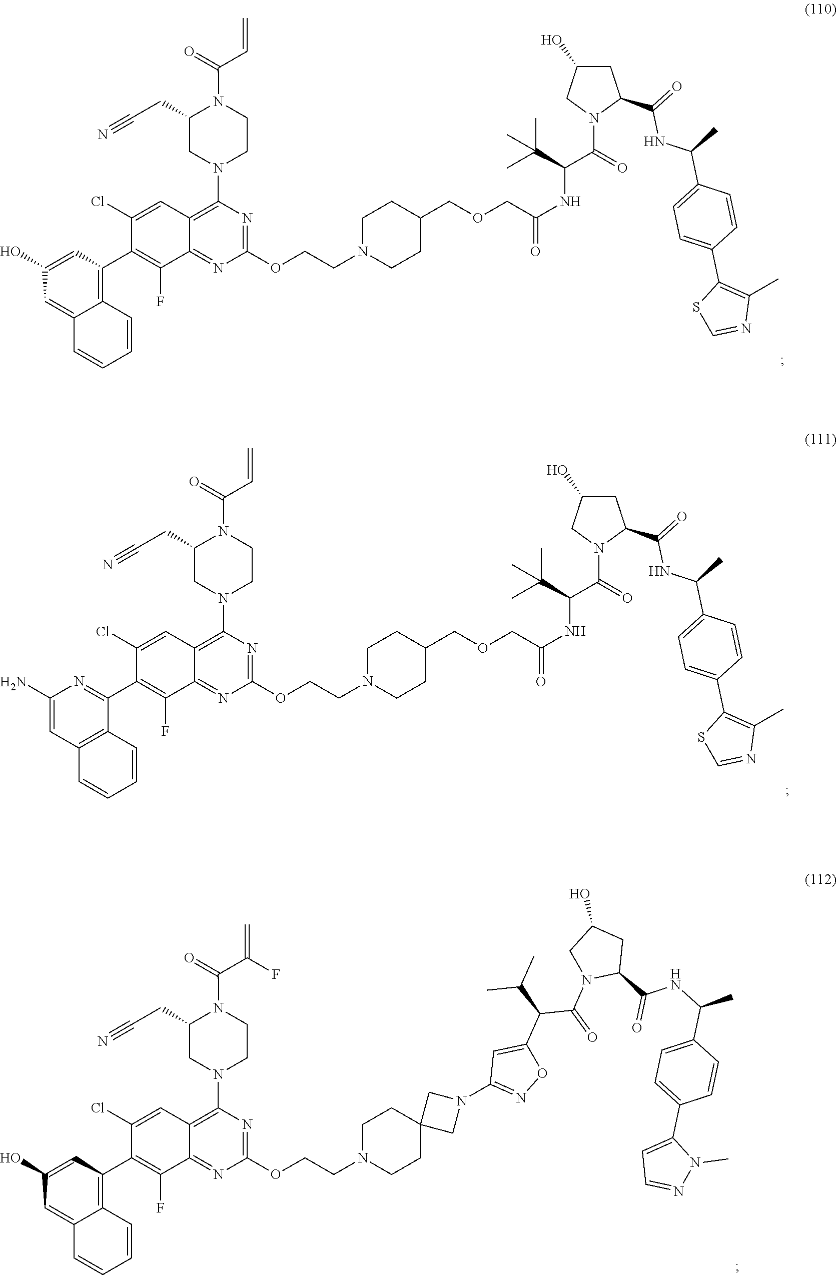

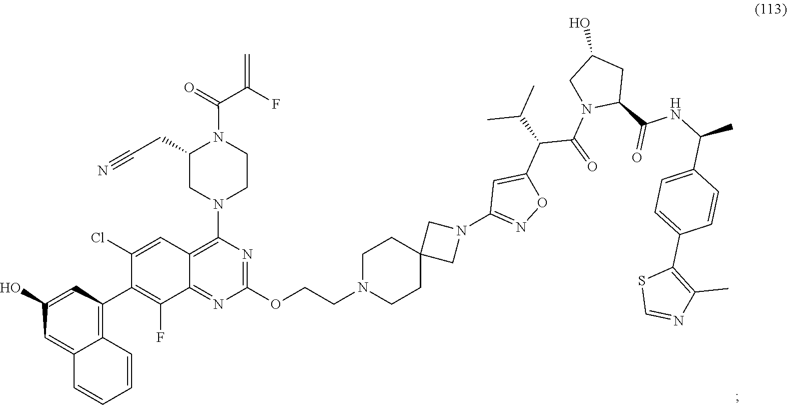

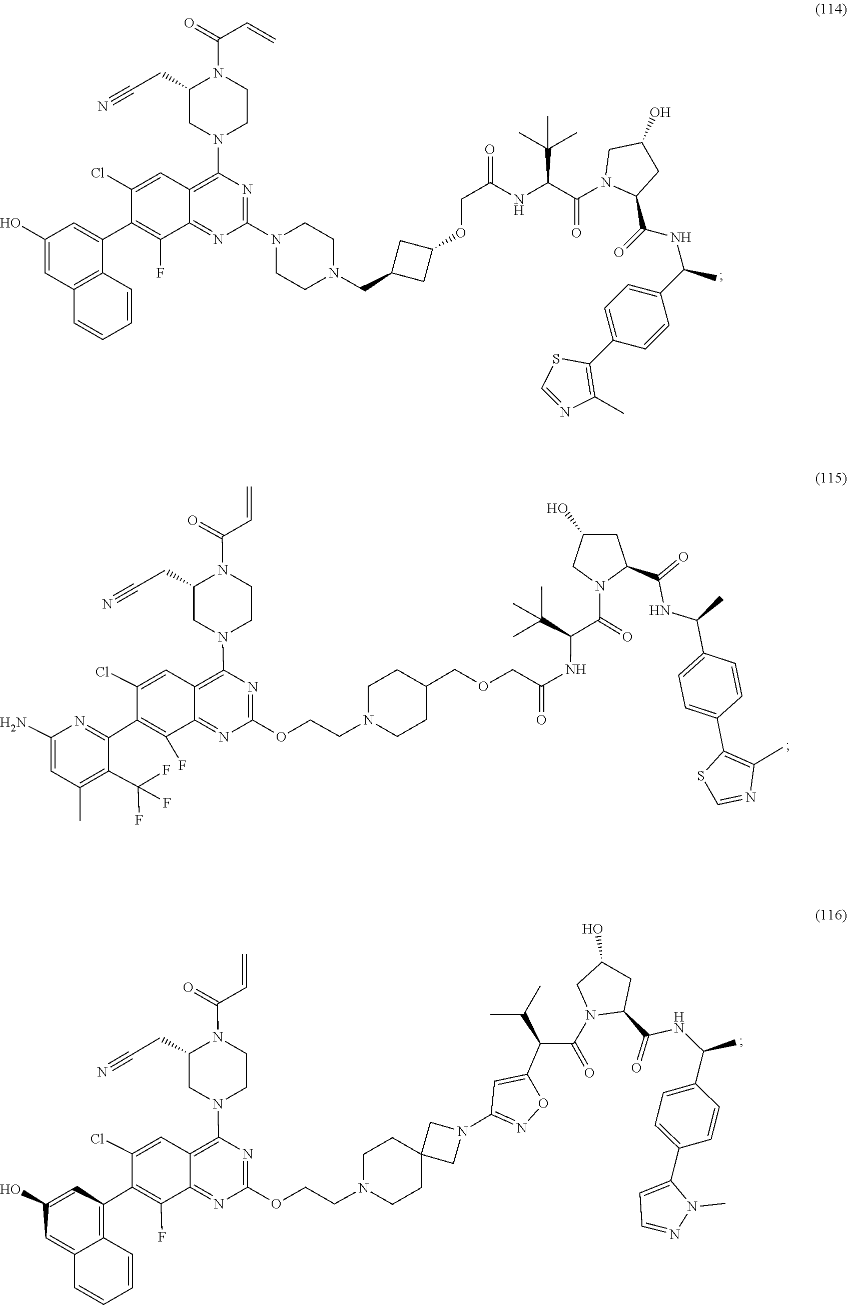

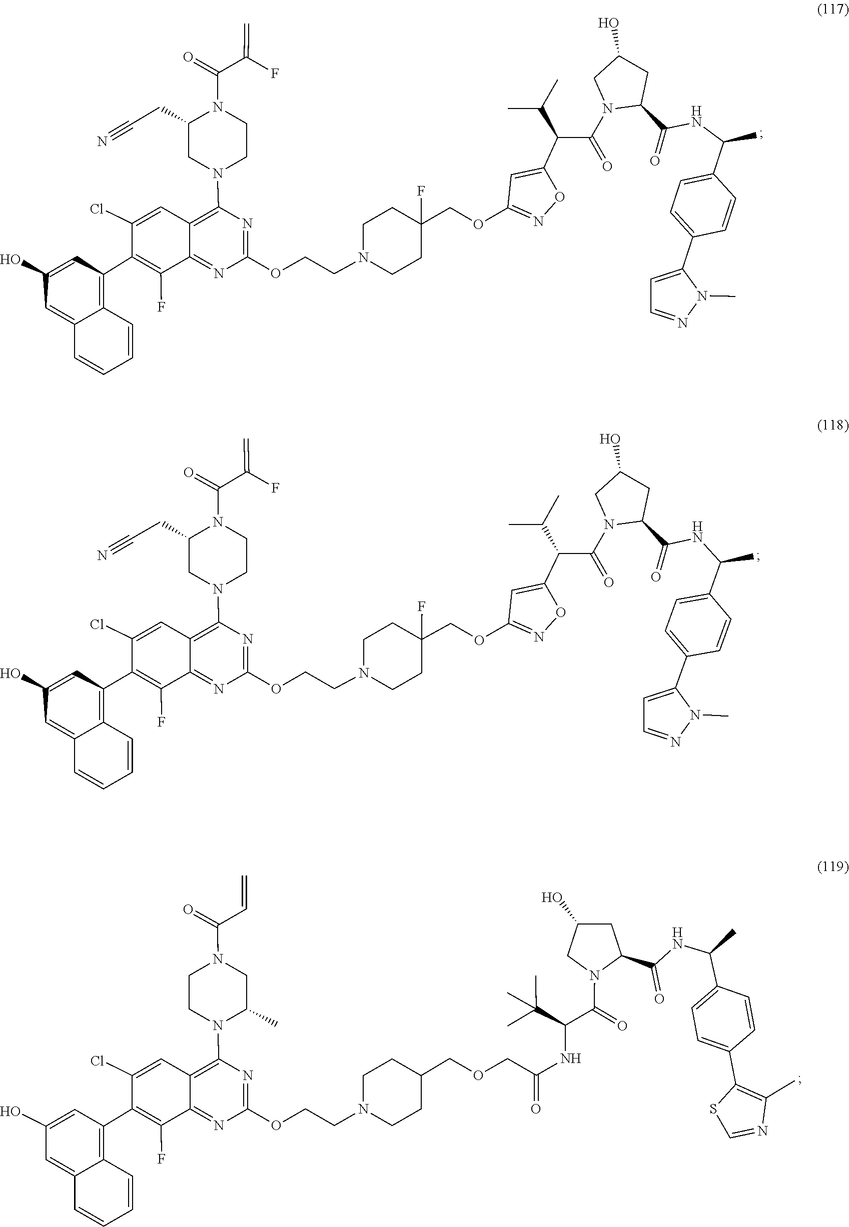

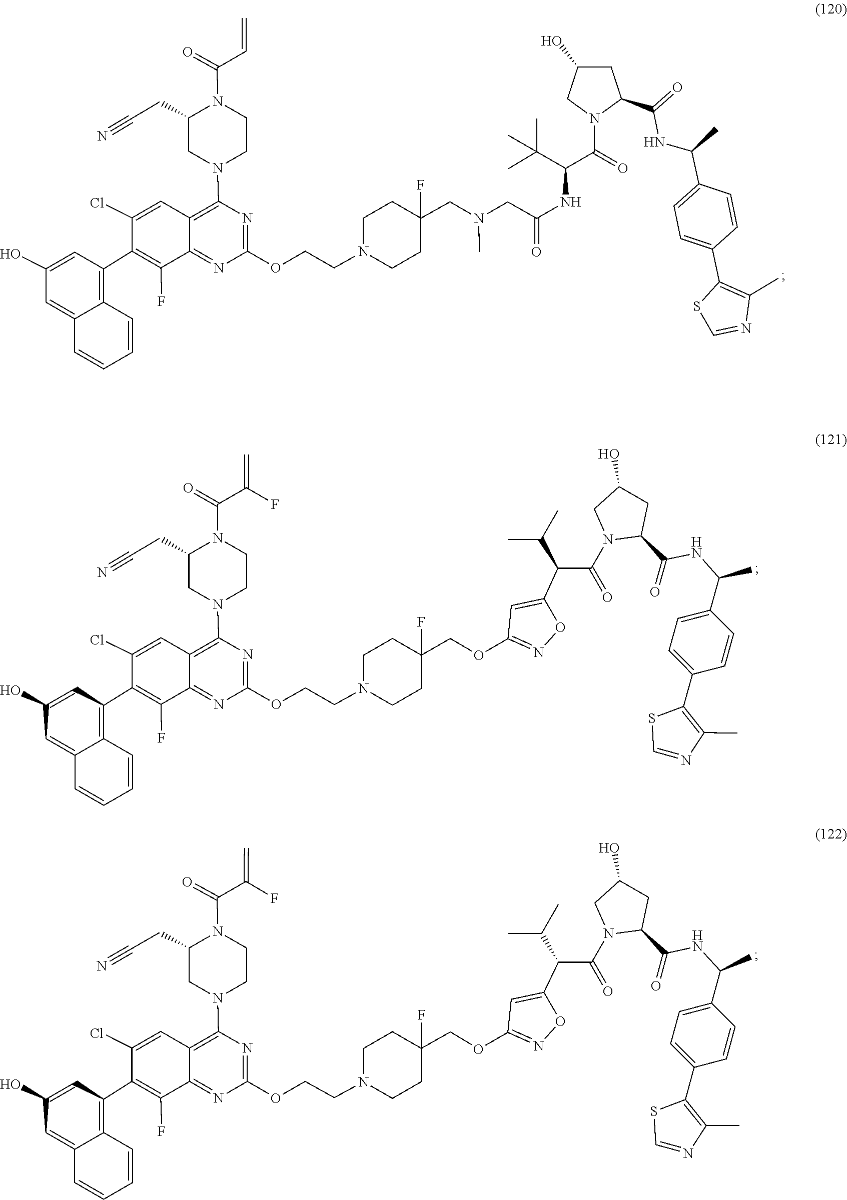

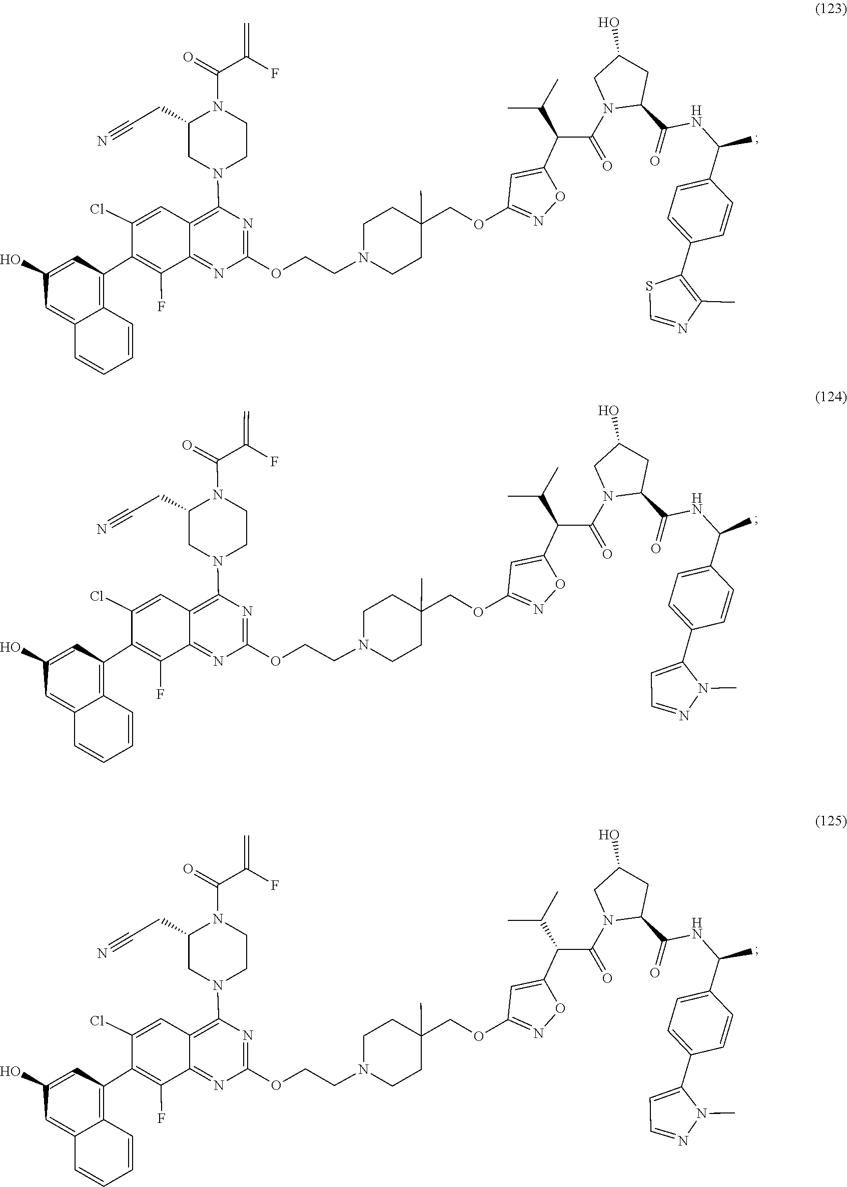

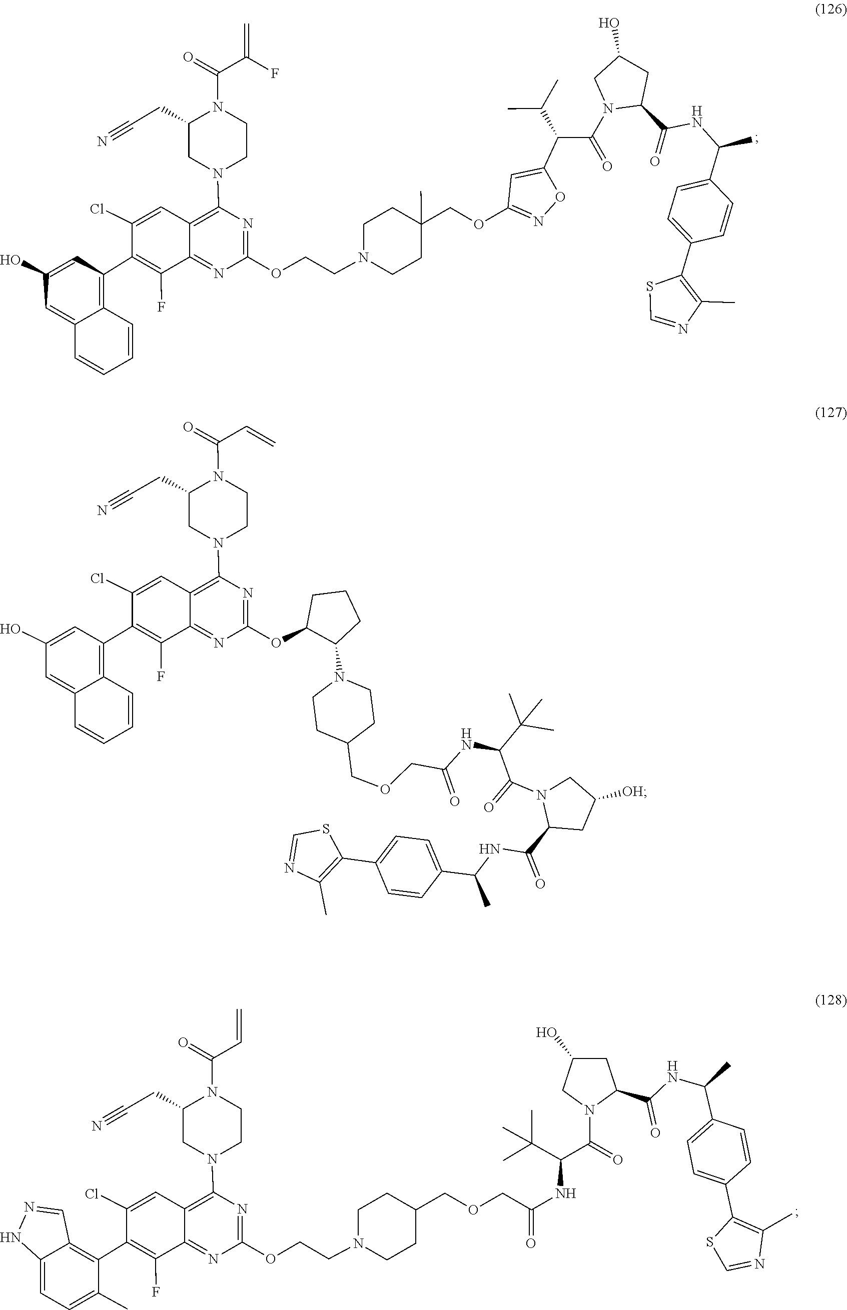

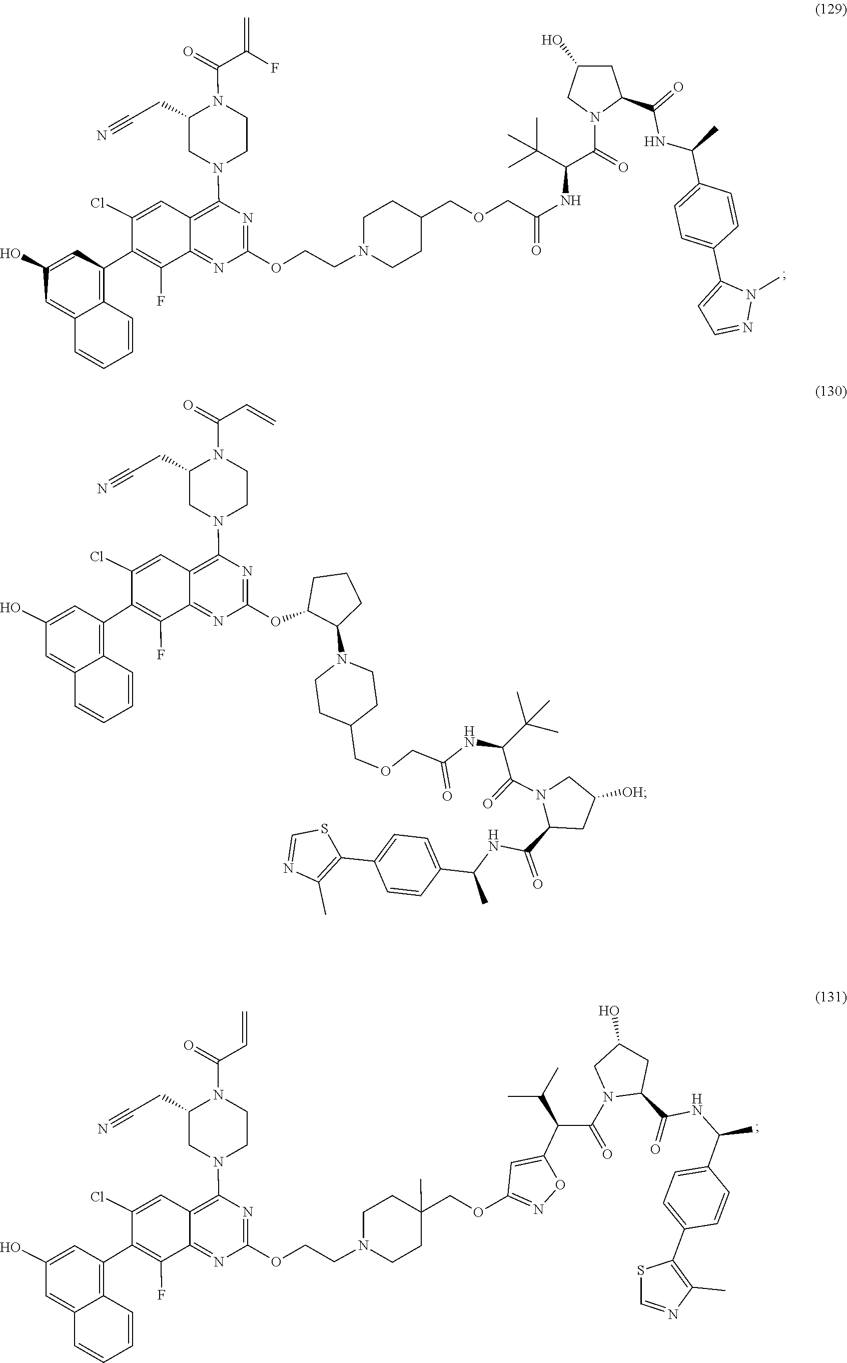

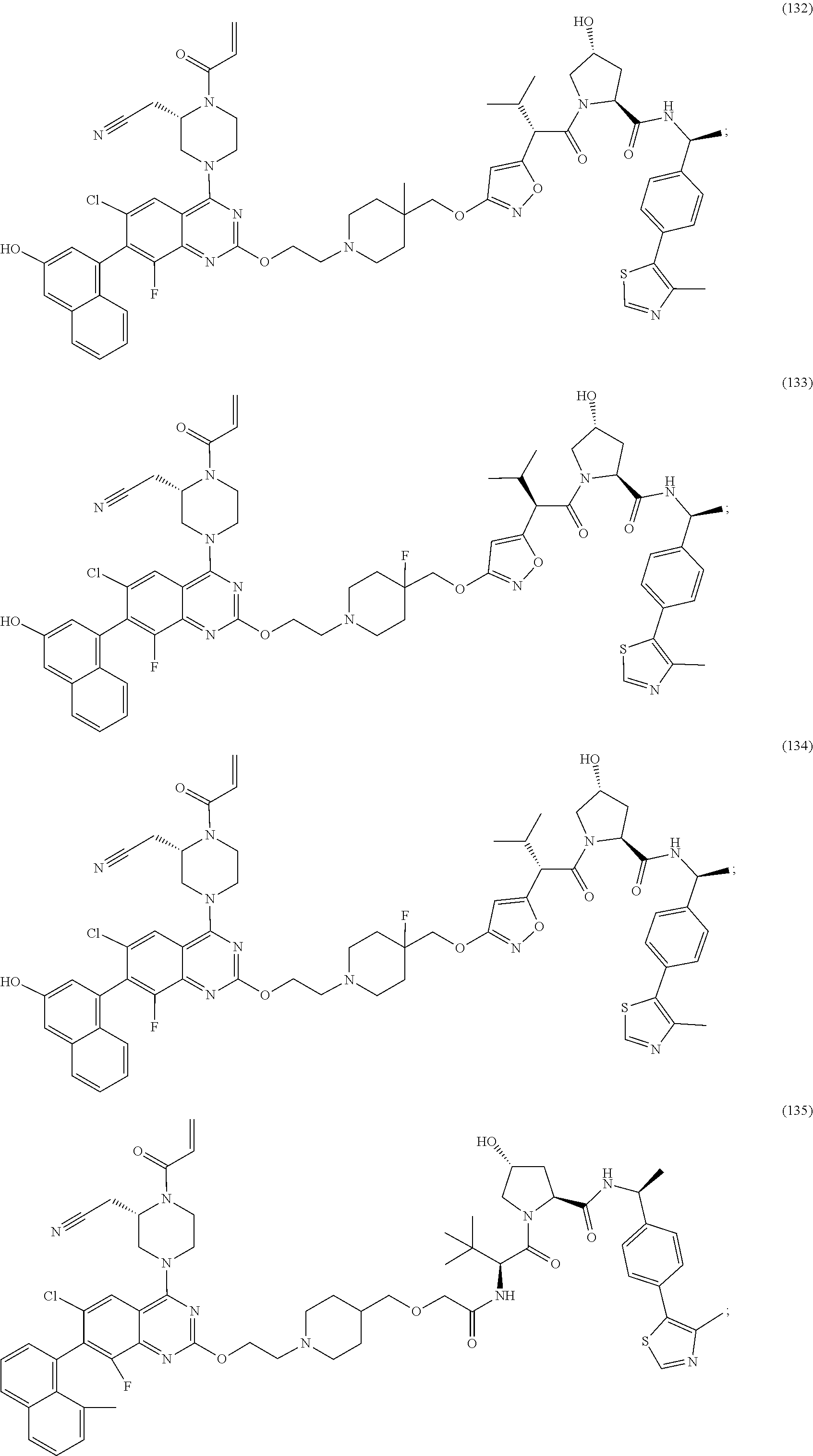

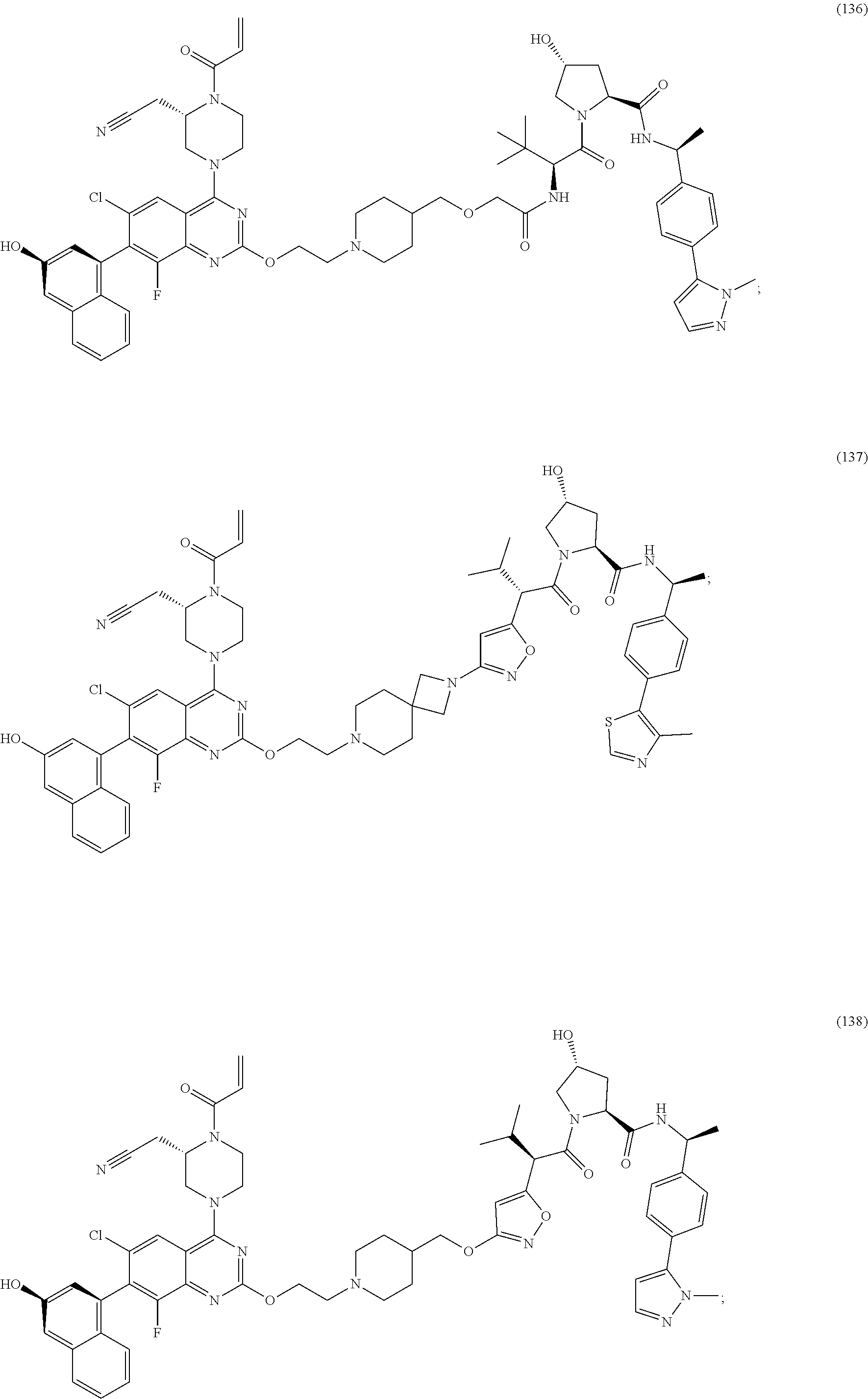

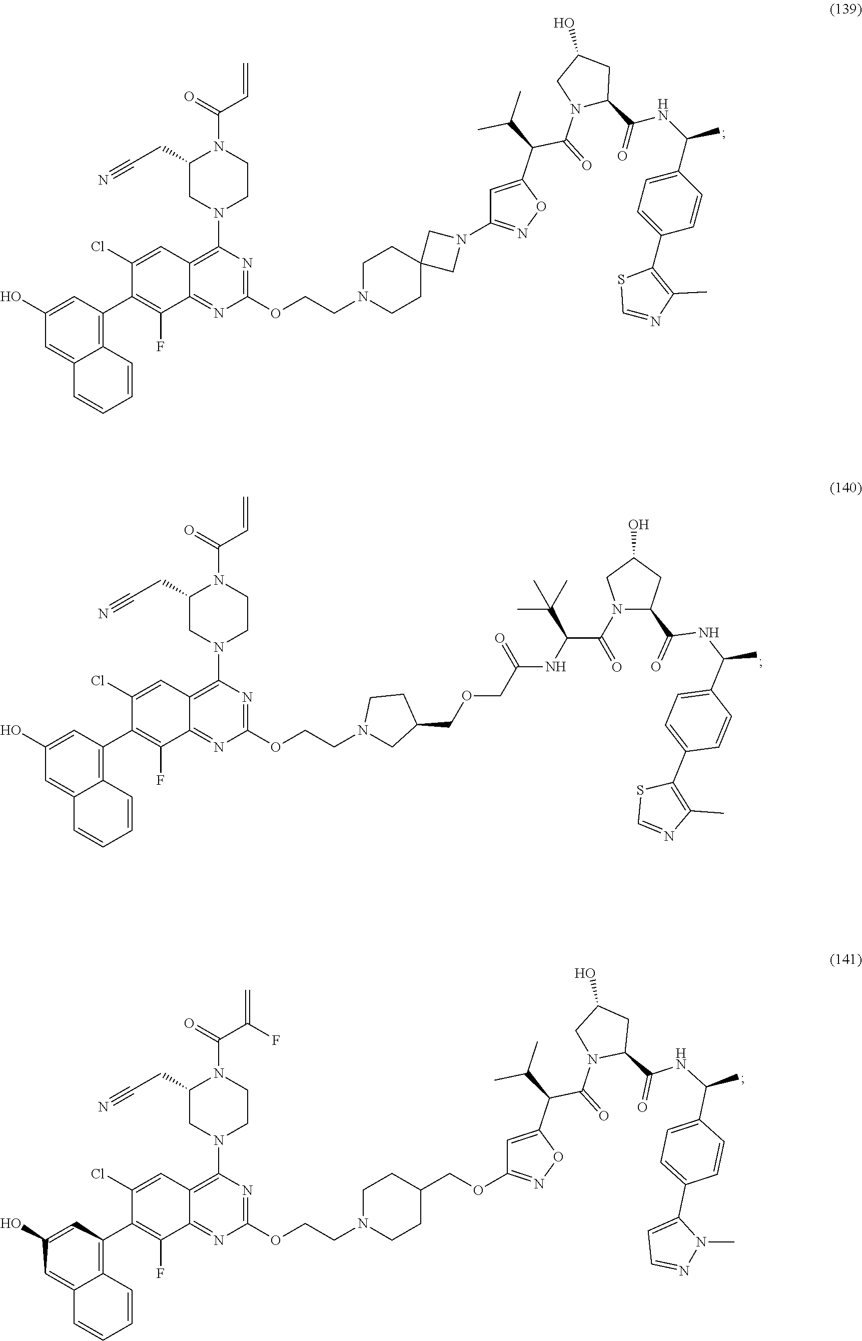

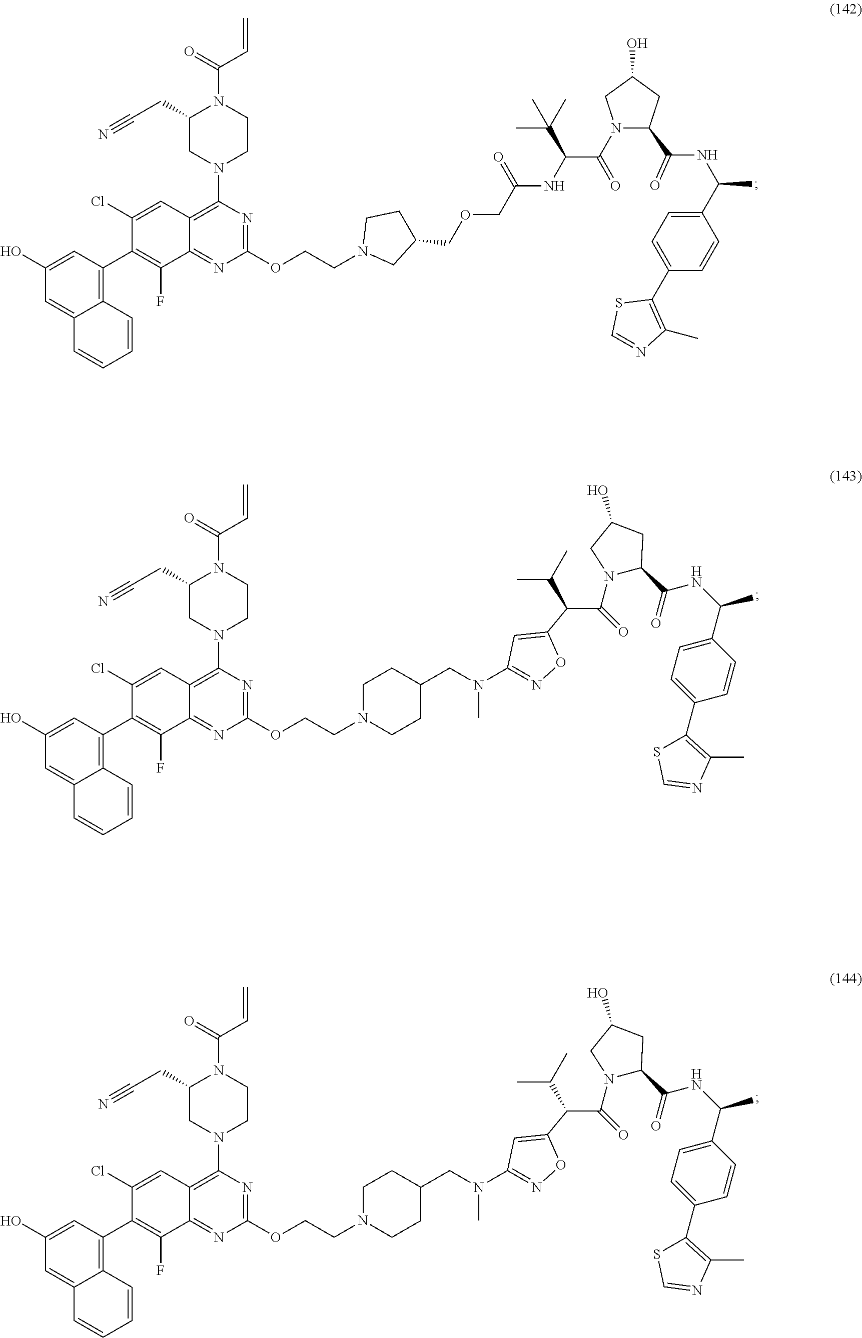

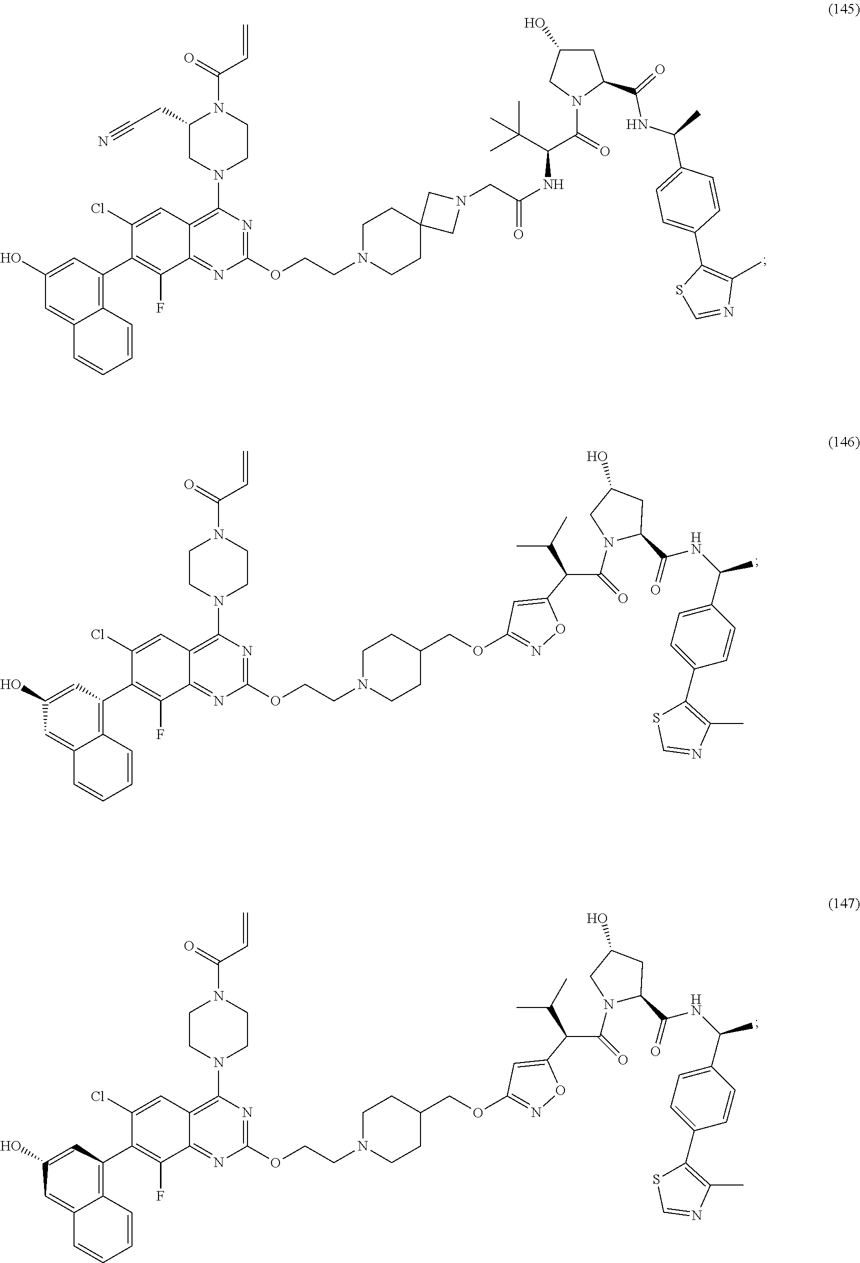

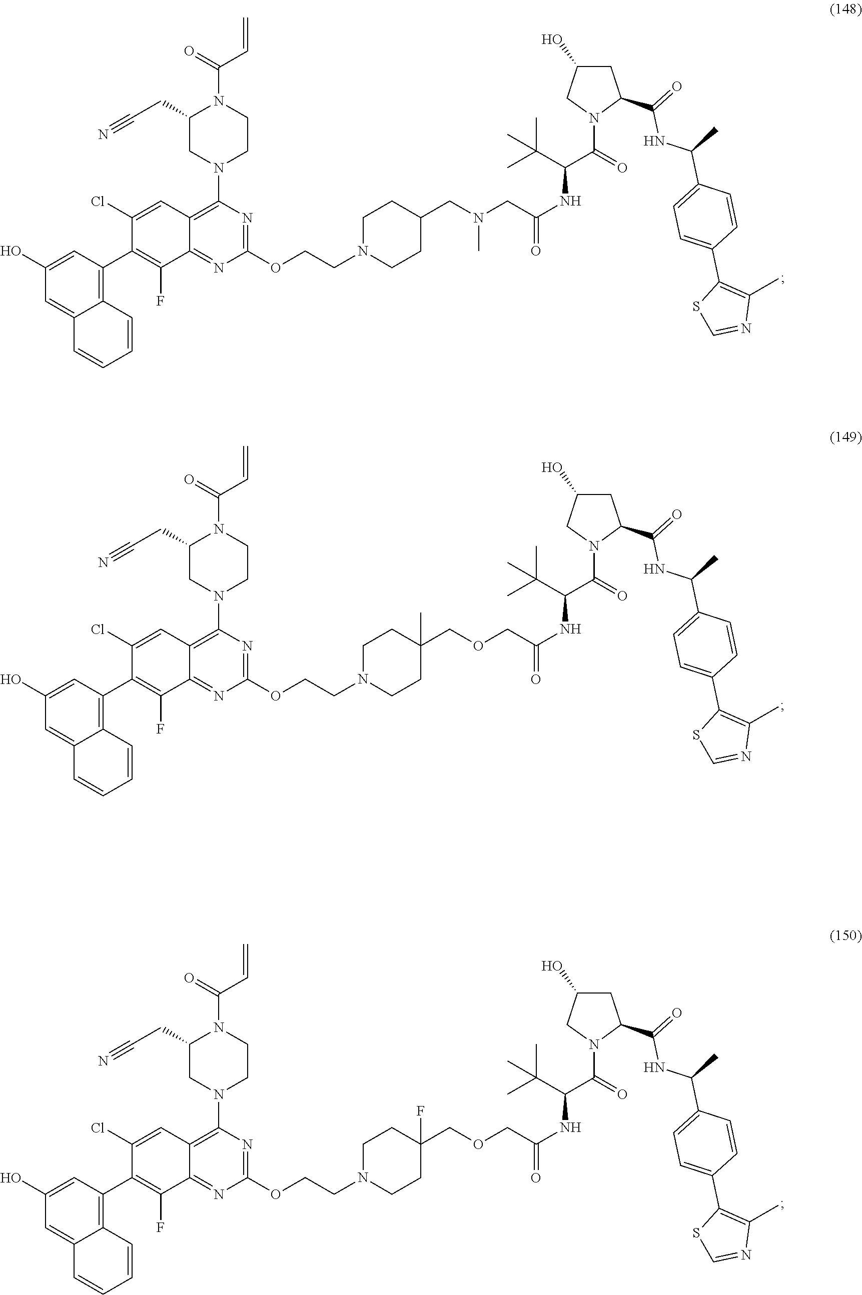

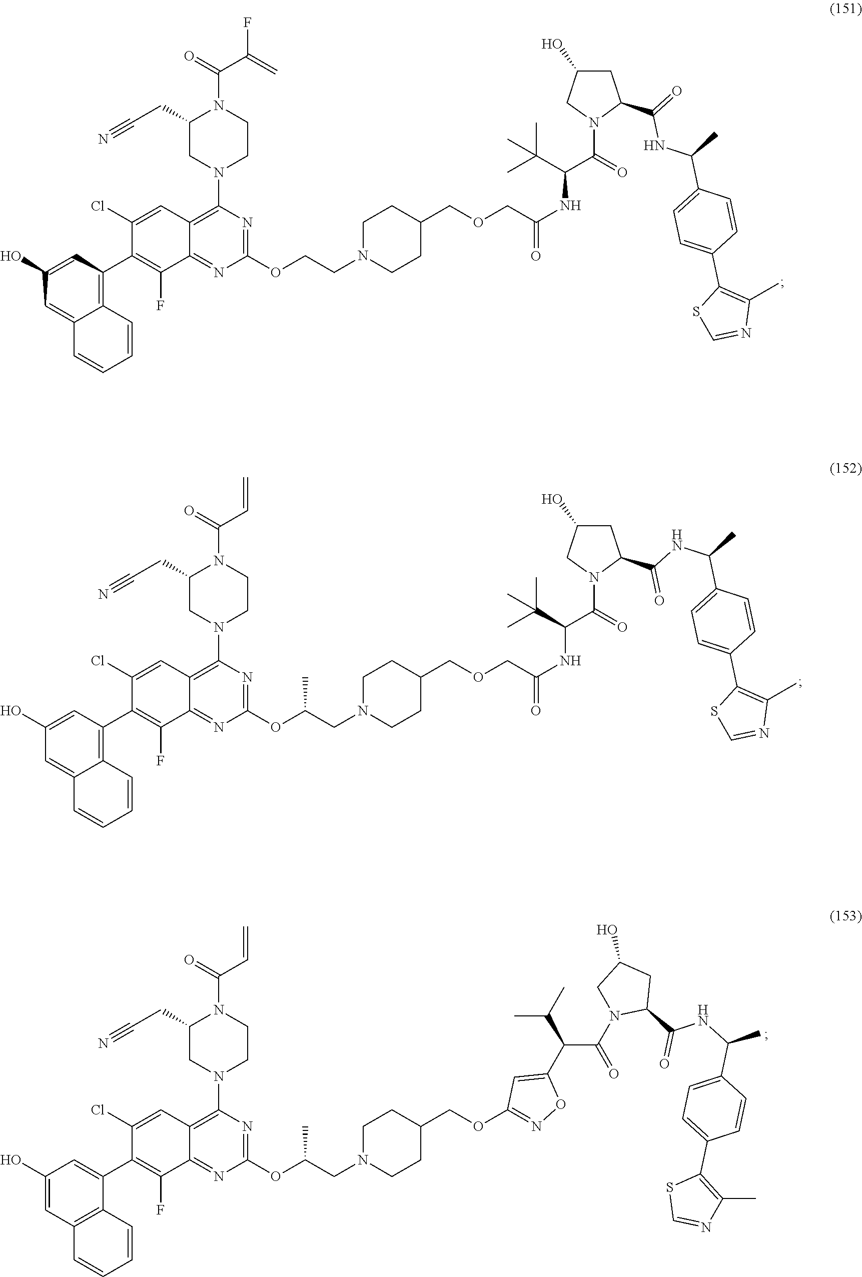

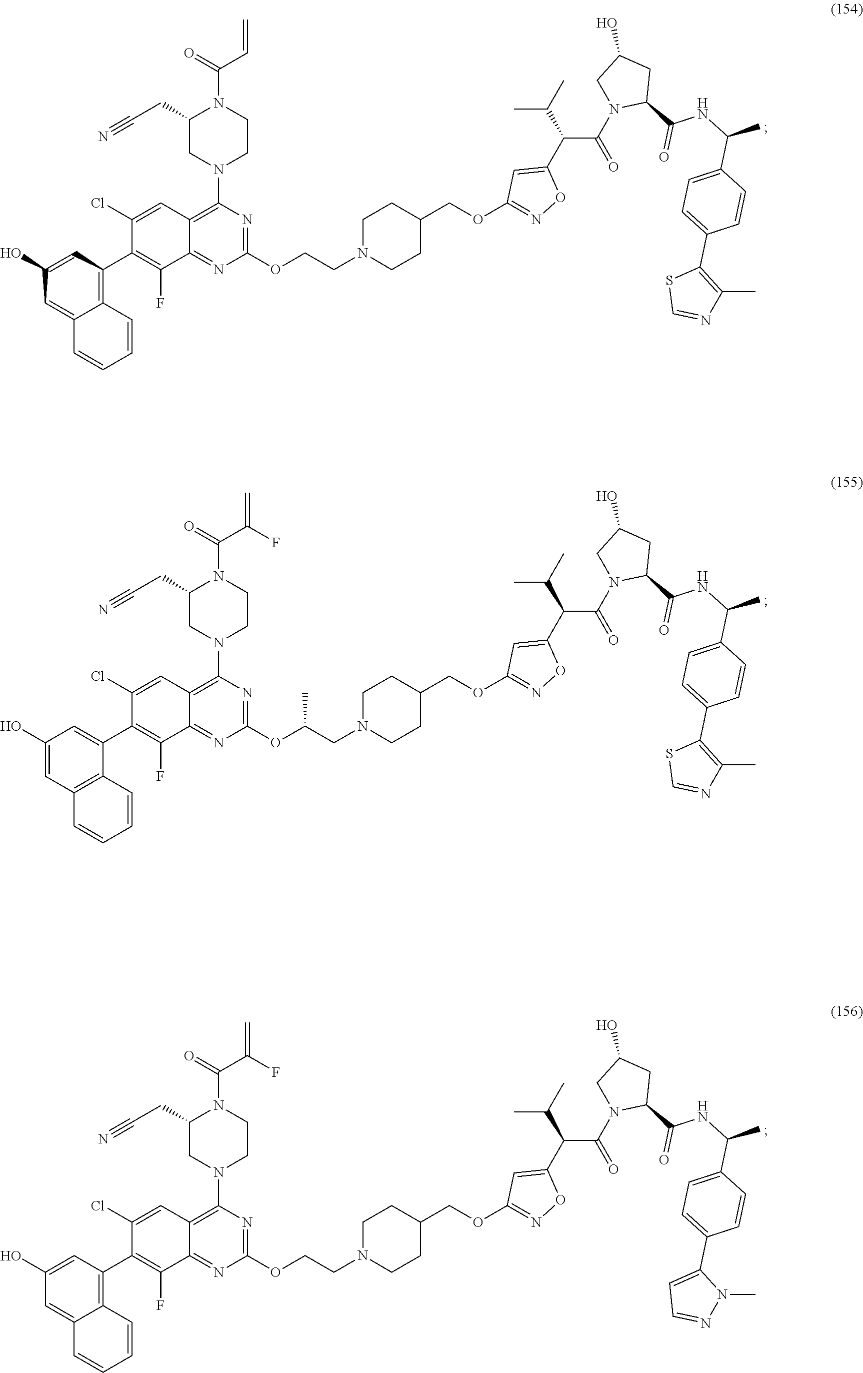

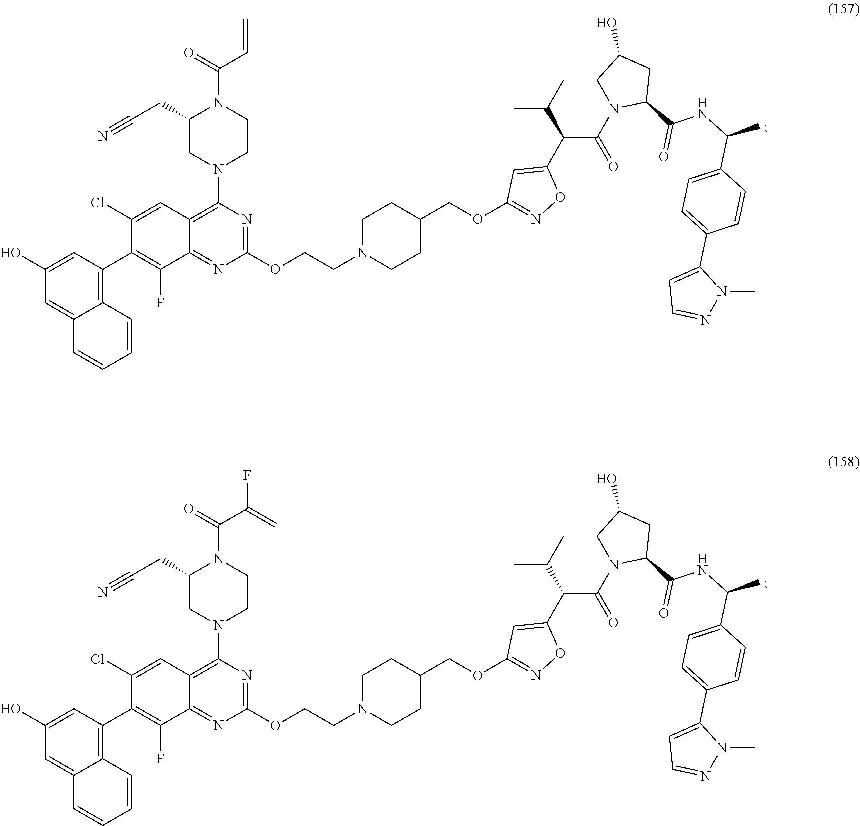 [SMILES: C=CC(=O)N1CCN(c2nc(OCCN3CCC(COc4cc([C@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N ; C=C(F)C(=O)N1CCN(c2nc(OCCN3CCC(COc4cc([C@@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N]

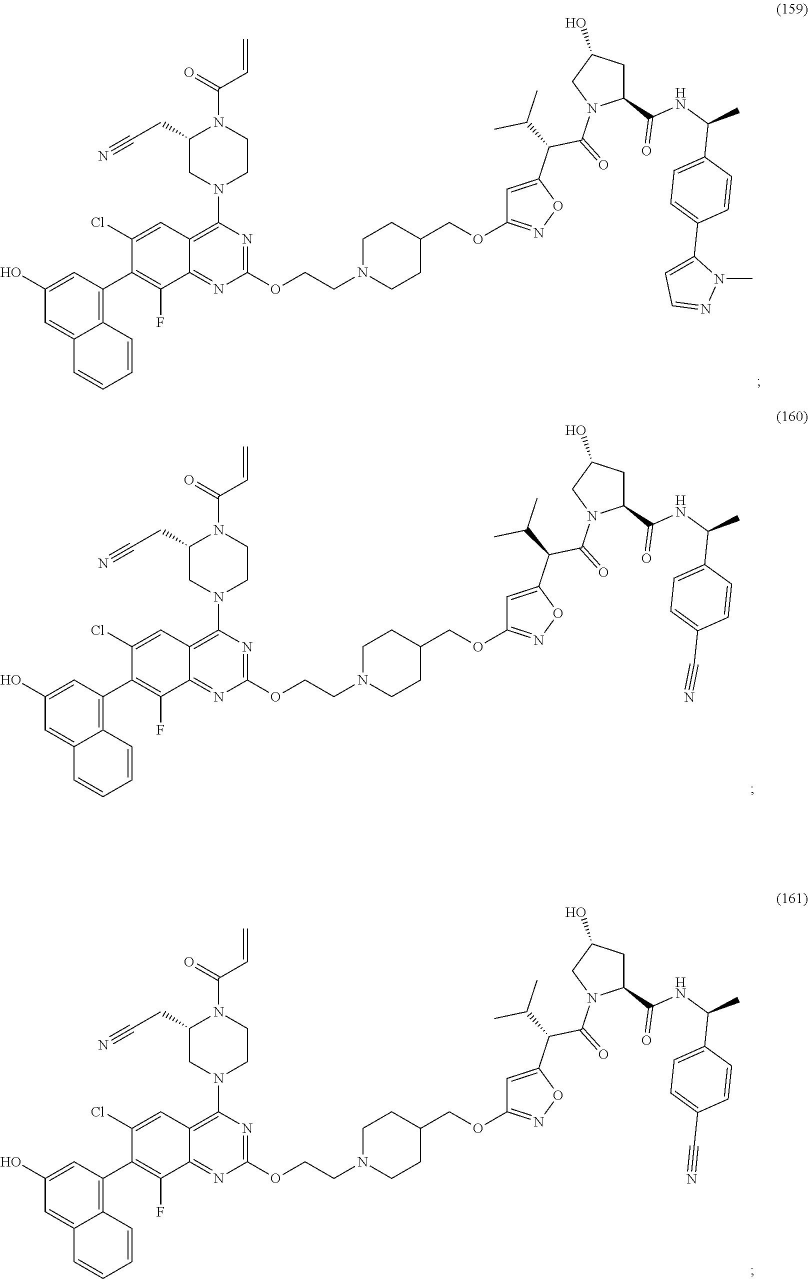 [SMILES: C=CC(=O)N1CCN(c2nc(OCCN3CCC(COc4cc([C@@H](C(=O)N5C[C@H](O)C[C@H]5C(=O)N[C@@H](C)c5ccc(-c6ccnn6C)cc5)C(C)C)on4)CC3)nc3c(F)c(-c4cc(O)cc5ccccc45)c(Cl)cc23)C[C@@H]1CC#N ; C=CC(=O)N1CCN(c2nc(OCCN3CCC(COc4cc([C@H](C(=O)N5C[C@H](O)C[C@H]5C(=O)N[C@@H](C)c5ccc(C#N)cc5)C(C)C)on4)CC3)nc3c(F)c(-c4cc(O)cc5ccccc45)c(Cl)cc23)C[C@@H]1CC#N ; C=CC(=O)N1CCN(c2nc(OCCN3CCC(COc4cc([C@@H](C(=O)N5C[C@H](O)C[C@H]5C(=O)N[C@@H](C)c5ccc(C#N)cc5)C(C)C)on4)CC3)nc3c(F)c(-c4cc(O)cc5ccccc45)c(Cl)cc23)C[C@@H]1CC#N]

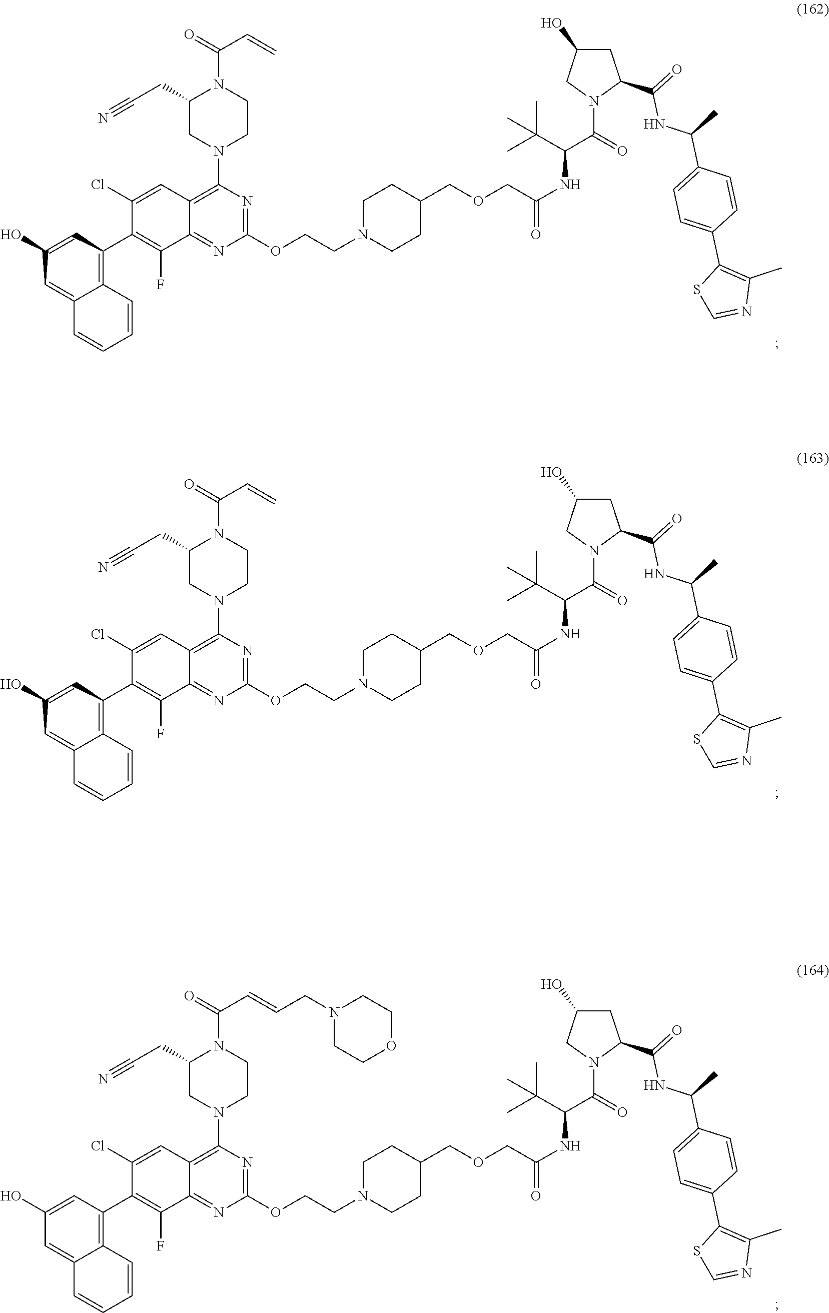

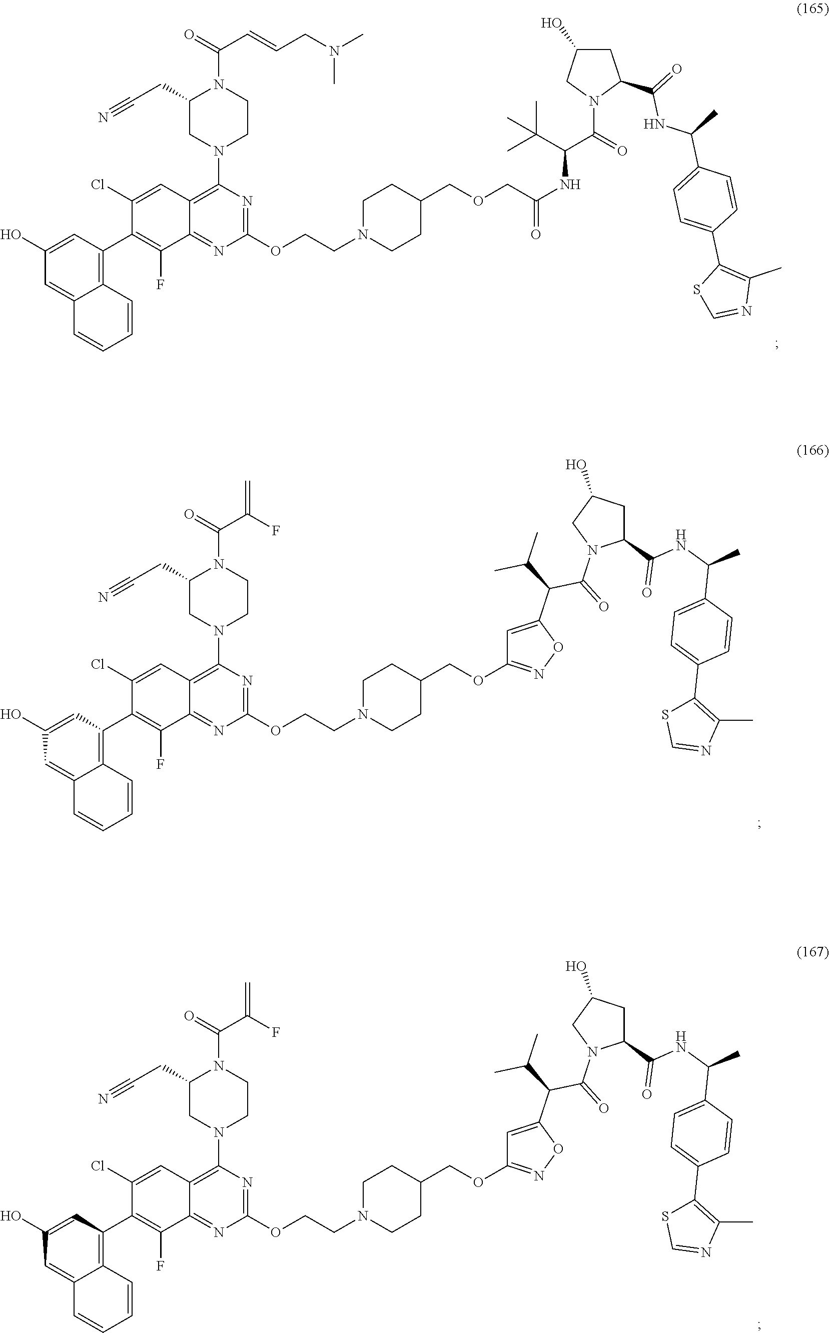

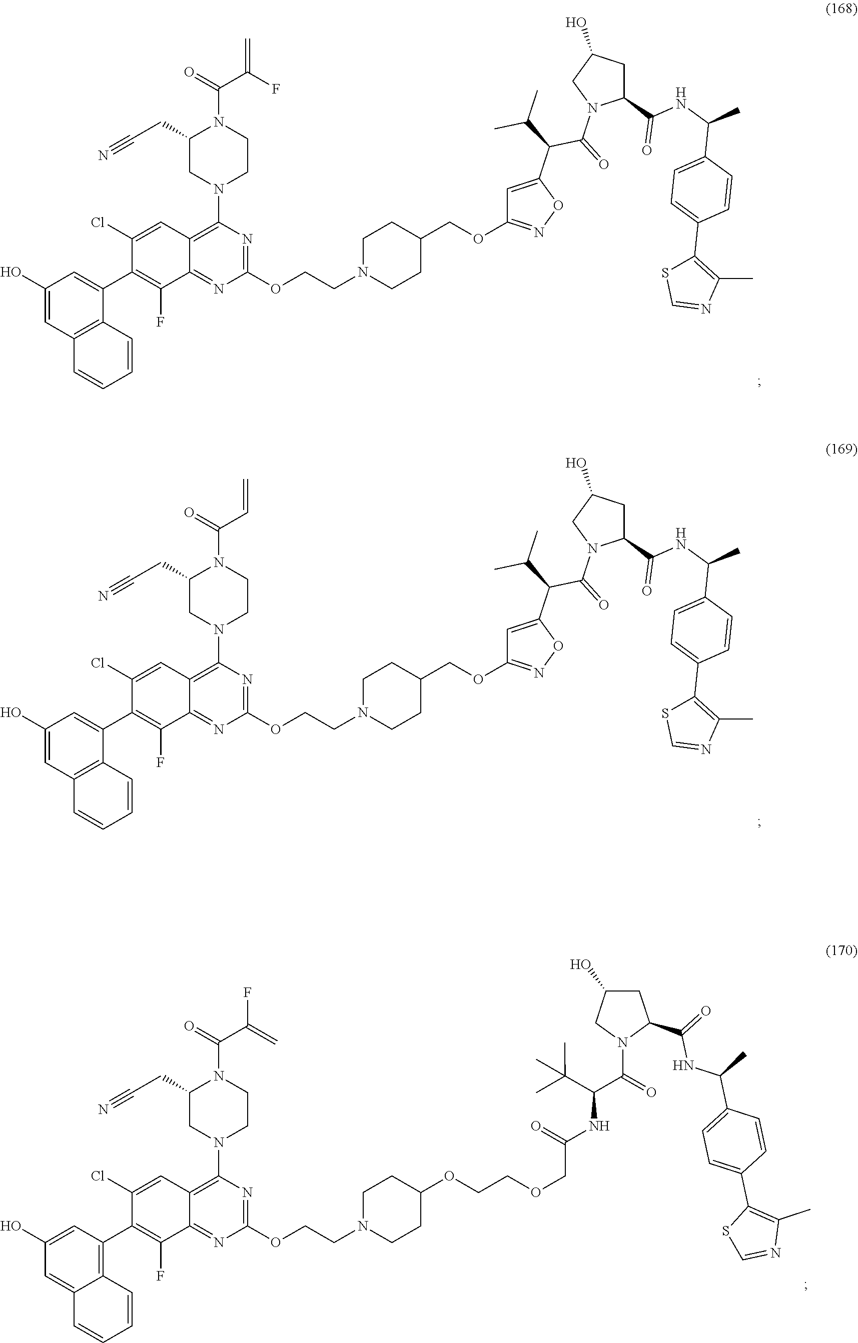

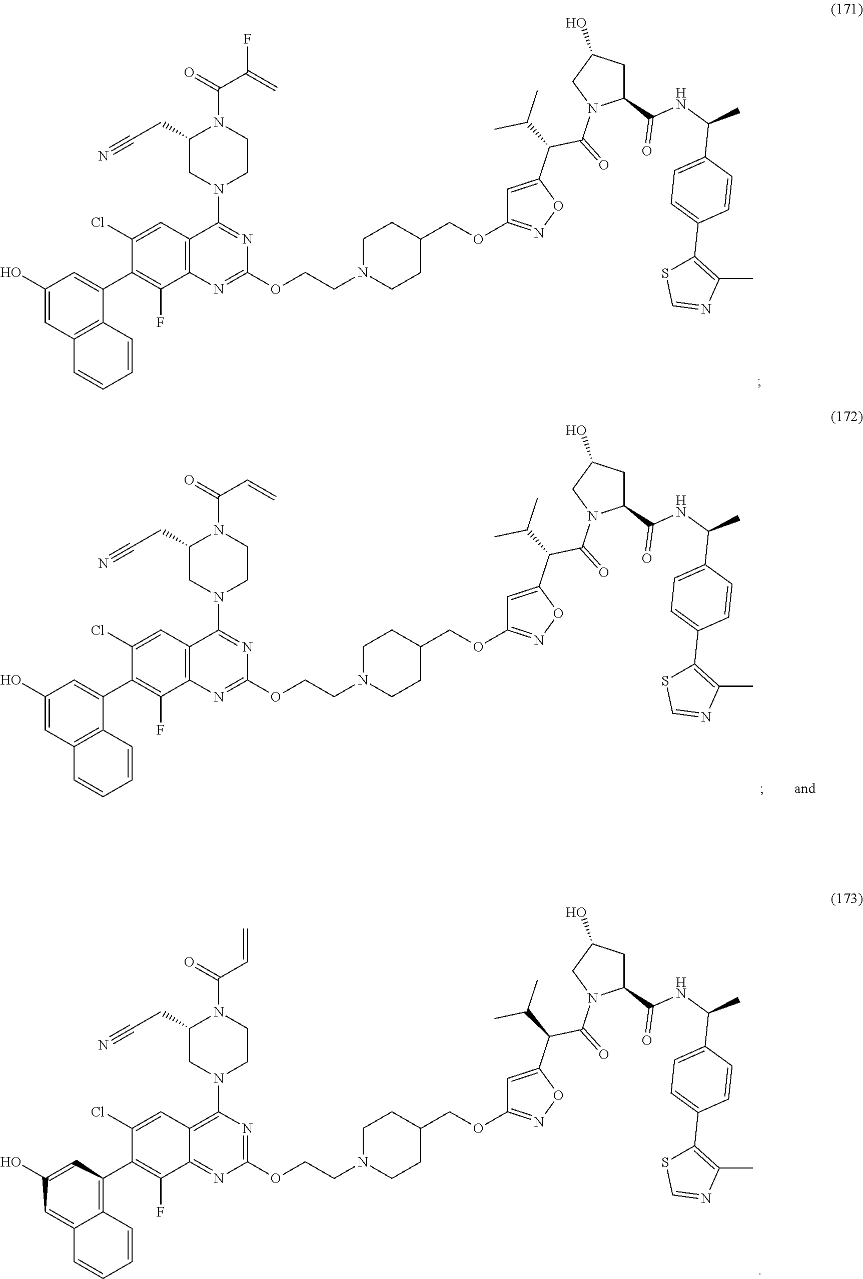

22. A composition comprising an effective amount of a bifunctional compound of claim 1, and a pharmaceutically acceptable carrier.

23. The composition of claim 22, wherein the composition further comprises at least one of additional bioactive agent or a second bifunctional compound.

24. The composition of claim 23, wherein the additional bioactive agent is an anti-inflammatory, a chemotherapy agent, or an immunomodulatory agent.

25. A composition comprising a pharmaceutically acceptable carrier and an effective amount of at least one compound of claim 1 for treating a disease, a disorder or a symptom casually related to KRas in a subject, wherein the composition is effective in treating or ameliorating the disease, disorder, or at least one symptom of the disease or disorder.

26. The composition of claim 25, wherein the disease or disorder is pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

27. A method of treating or preventing a disease, a disorder, or symptom associated with KRas comprising, providing a patient in need thereof, and administering an effective amount of a compound as described herein or composition comprising the same to the patient, wherein the compound or composition is effective in treating or ameliorating the disease, disorder, or at least one symptom of the disease or disorder.
